(19) United States
(12) Patent Application Publication
(10) Pub. No.: US 2024/0059671 A1
(43) Pub. Date: Feb. 22, 2024

(54) TYROSINE KINASE 2 (TYK2) DEGRADATION COMPOUNDS AND METHODS OF USE

(71) Applicant: CULLGEN (SHANGHAI), INC., Shanghai (CN)

(72) Inventors: Jing LIU, Oradell, NJ (US); Liqun CHEN, Shanghai (CN); Jialiang WANG, Nashville, TN (US); Xiaoran HAN, Shanghai (CN); Zhaohui WU, Germantown, TN (US); Ting YANG, Shanghai (CN); Chengwei ZHANG, Shanghai (CN)

(21) Appl. No.: 18/252,737

(22) PCT Filed: Nov. 12, 2021
(86) PCT No.: PCT/CN2021/130434

§ 371 Date: May 12, 2023

(30) Foreign Application Priority Data
Nov. 12, 2020 (WO) PCT/CN2020/128350

(51) Int. Cl. C07D 401/14; C07D 417/14

(52) U.S. Cl. CPC C07D 401/14; C07D 417/14

(57) ABSTRACT

This disclosure relates to heterobifunctional compounds (e.g., bi-functional small molecule compounds), compositions comprising one or more of the heterobifunctional compounds, and to methods of use the heterobifunctional compounds for the treatment of certain disease in a subject in need thereof. The disclosure also relates to methods for identifying such heterobifunctional compounds.

### TECHNICAL FIELD

The present disclosure belongs to the field of medicine, and specifically relates to tyrosine kinase 2 (tyk2) degradation compounds and methods of use.

### BACKGROUND OF THE INVENTION

The Janus kinases (JAKs) are key signal transduction molecules orchestrating the cytokine-induced signaling network. JAKs are non-receptor tyrosine kinases comprising 4 members, JAK1/2/3 and TYK2. Upon cytokine binding, JAKs are recruited to the cytoplasmic tails of cytokine receptors, and induce phosphorylation of each other and also these receptors. Subsequently, activated JAKs phosphorylate the signal transducer and activator of transcription (STAT1-6) family transcription factors, leading to their dimerization, nuclear translocation, and consequently transcriptional activation of many genes implicated in cellular proliferation, survival, differentiation, immune response, and other important biological processes. Because of the central roles of JAKs in innate and adaptive immunity, they are actively pursued by the pharmaceutical industry for the treatment of immunological disorders and cancers. In recent years, a growing number of JAK kinase inhibitors have reached the market, including ruxolitinib, a JAK1/2 dual inhibitor for the treatment of myelofibrosis and polycythemia vera, and fedratinib, also for myelofibrosis. Baricitinib is another JAK1/2 dual inhibitor for the treatment of rheumatoid arthritis (RA), atopic dermatitis and systemic lupus erythematosus. Tofacitinib is a pan-JAK inhibitor for the treatment of patients with moderate to severe RA, psoriatic arthritis, and ulcerative colitis. However, despite significant therapeutic efficacy in some autoimmune diseases and cancers, the general immunosuppressive effects of JAK kinase inhibitors are substantial.

TYK2 is the first identified JAK kinase, but has not been studied as extensively as other JAKs until recent. TYK2 shares the seven Janus homology domains (JH1-7) with other family members. The carboxyl terminal JH1 domain contains the catalytic center. The neighboring JH2 domain is a pseudokinase domain that functions as a self-inhibitory domain. Once recruited to heterodimeric cytokine receptors, TYK2 generally partner with JAK1 or JAK2 for activating downstream STAT proteins. A growing body of studies has established essential roles of TYK2 in signaling induced by several key interleukins and interferons, particularly IL-12, IL-23, and type I interferons. TYK2 may also be implicated in signaling of IL-6 and IL-10. The links between TYK2 and these cytokines establish it as a potential therapeutic target in a variety of immunologic disorders, including rheumatoid arthritis, psoriasis, type I diabetes, systemic lupus erythematosus, ankylosing spondylitis, Crohn's disease, ulcerative colitis, multiple sclerosis, juvenile idiopathic arthritis, primary biliary cirrhosis, and inflammatory bowel disease (IBD). Aberrant activation of TYK2 is also found in cancers.

Pan-JAK kinase inhibitors have the potential to block TYK2 signaling. However, blockade of all JAK kinases severely compromise immune response that can lead to serious adverse events, such as infections and cancers. Genetically engineered models in rodents and inherited disease in human have informed sharply contrasting consequences of deficiency for individual JAK kinases. Loss of JAK1 or JAK2 in mouse is embryonically lethal, while depletion of JAK3 results in severe combined immunodeficiency. In contrast, mice lacking TYK2 are viable with impaired immune response but refractory to autoimmune diseases. Hence, selectively targeting TYK2 has significant potentials in autoimmune and inflammatory diseases but may not induce broad immunosuppression as pan-JAK inhibition does.

The preferred benefit to risk ratio of targeting TYK2 has increasingly attracted the interests of academia and pharmaceutical industry. Neutralizing antibodies against IL-12 and IL-23, the main cytokines that signal through TYK2, have been approved for treating psoriasis, psoriatic arthritis and Crohn's disease. Over the past decade, a variety of TYK2 kinase inhibitors with varying degree of selectivity over other JAK family members have been reported and patented. Some of these TYK2 inhibitors have proceeded into different clinical stages.

While TYK2 and other JAK kinase inhibitors hold promises treating a wide range of immunologic and malignant condition, small molecule inhibitors primarily modulate the catalytic activities of these kinases. However, TYK2 can contribute to cytokine signaling through its scaffolding functions. Kinase-dead TYK2 mutants retain the ability to regulate stability of receptors of type I interferon. The catalytic functions of TYK2 are also dispensable for activation of PI3K signaling. Therefore, depletion of TYK2 using small molecule degraders may have more profound impact on cytokine response than kinase inhibitors.

Currently available small molecules targeting TYK2 focus on inhibition of TYK2 kinase activities.

There is a need in the art for compounds, compositions, and methods of use of the compounds for the treatment of diseases in a subject in need thereof.

### SUMMARY OF THE INVENTION

This disclosure relates to heterobifunctional compounds (e.g., bi-functional small molecule compounds), compositions comprising one or more of the heterobifunctional compounds, and to methods of use of the heterobifunctional compounds for the treatment of certain diseases in a subject in need thereof. The disclosure also relates to methods for identifying such heterobifunctional compounds.

According to the first aspect of the present disclosure, a heterobifunctional compound disclosed herein comprises a Tyrosine Kinase 2 (TYK2) ligand conjugated to a degradation tag, or a pharmaceutically acceptable salt, hydrate, solvate, prodrug, stereoisomer, tautomer, or analog thereof.

In one embodiment, the TYK2 ligand binds to the Janus Homology 2 (JH2) domain of TYK2.

In another embodiment, the degradation tag binds to a ubiquitin ligase or is a hydrophobic group or a tag that leads to misfolding of the TYK2 proteins. In another embodiment, the ubiquitin ligase is an E3 ligase. In another embodiment, the E3 ligase is selected from the group consisting of a VHL E3 ligase, a cereblon E3 ligase, an IAP ligase, a MDM2 ligase, a TRIM24 ligase, a TRIM21 ligase, a KEAP1 ligase, DCAF16 ligase, RNF4 ligase, RNF114 ligase, and AhR ligase.

In another embodiment, the degradation tag is selected from the group consisting of VHL-1, pomalidomide, thalidomide, lenalidomide, adamantane, 1-((4,4,5,5,5-pentafluoropentyl)sulfinyl)nonane, nutlin-3a, RG7112, RG7338, AMG232, AA-115, bestatin, MV-1, LCL161, CPD36, GDC-0152, CRBN-1, CRBN-2, CRBN-3, CRBN-4, CRBN-5, CRBN-6, CRBN-7, CRBN-8, CRBN-9, CRBN-10, CRBN-11, CRBN-12, CRBN-13, CRBN-14, CRBN-15, CRBN-16, and analogs thereof.

In another embodiment, the TYK2 ligand is conjugated to the degradation tag via a linker moiety.

In another embodiment, the heterobifunctional compound disclosed herein comprises a moiety of FORMULA I;

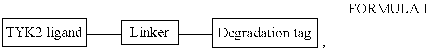

or a pharmaceutically acceptable salt, hydrate, solvate, prodrug, stereoisomer, tautomer, or analog thereof, wherein
i) the TYK2 ligand comprises a moiety of FORMULA 1 or FORMULA 2:

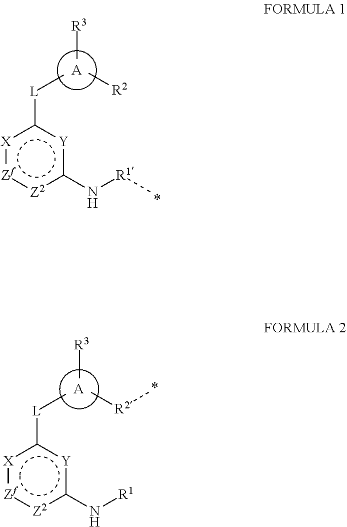

wherein *, Ring A, R¹, R^{1′}, R², R^{2′}, R³, X, Y, Z¹, Z² and L are defined as hereinafter;
ii) the degradation tag is a moiety of FORMULA 6A, 6B, and 6C:

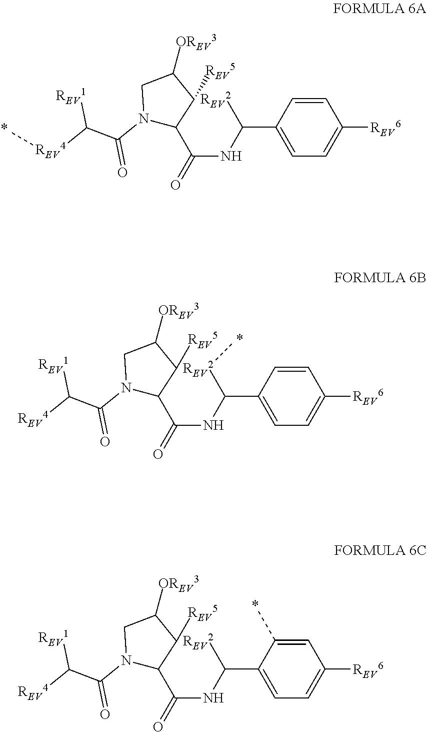

wherein *, R_{EV}¹, R_{EV}², R_{EV}^{2′}, R_{EV}³, R_{EV}⁴, R_{EV}^{4′}, R_{EV}⁵, and R_{EV}⁶ are defined as hereinafter;
or, the degradation tag is a moiety of FORMULA 5, and the degradation tag is connected to the linker moiety of the heterobifunctional compound via Z_{E};

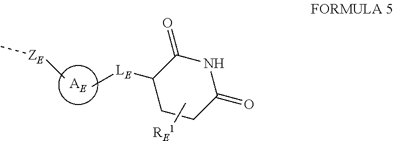

wherein Ring A_{E}, L_{E}, Z_{E}, and R_{E}¹ are defined as hereinafter;
and iii) the linker moiety is of FORMULA 9:

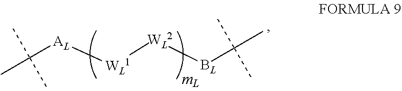

wherein m_{L}, A_{L}, W_{L}¹, W_{L}² and B_{L} are defined as hereinafter.

In some embodiments, the heterobifunctional compound is selected from the group consisting of CPD-001 to CPD-199 or a pharmaceutically acceptable salt or analog thereof. In some embodiments, the heterobifunctional compound is selected from the group consisting of CPD-038, CPD-039, CPD-040, CPD-047, CPD-084, CPD-085, CPD-099, CPD-100, CPD-110, CPD-112, CPD-114, CPD-115, CPD-121, CPD-124, CPD-125, CPD-126, CPD-127, CPD-131, CPD-133, CPD-134, CPD-143, CPD-144, CPD-148, CPD-150, CPD-151, CPD-155, CPD-157, CPD-158, CPD-159, CPD-164, CPD-167, CPD-175, and a pharmaceutically acceptable salt or analog thereof.

According to the 2nd aspect of the present disclosure, a pharmaceutical composition is provided herein comprising a compound according to the 1^{st} aspect of the present disclosure, and one or more pharmaceutically acceptable carriers. In one embodiment, the pharmaceutical composition further comprising one or more additional therapeutic agent.

According to the 3rd aspect of the present disclosure, a method of treating and/or preventing a TYK2-mediated disease provided herein comprises administering to a subject in need the heterobifunctional compound or a pharmaceutically acceptable salt or analog thereof.

In one embodiment, the subject in need means a subject with one or more TYK2-mediated diseases and/or a subject with elevated TYK2 function.

In one embodiment, the TYK2-mediated disease results from TYK2 expression, mutation, deletion, or fusion.

In one embodiment, the subject with the TYK2-mediated disease has an elevated TYK2 function relative to a healthy subject without the TYK2-mediated disease.

In one embodiment, the subject is mammal, preferably, human.

In one embodiment, the heterobifunctional compound is selected from the group consisting of CPD-001 to CPD-199, or analogs thereof.

In one embodiment, the heterobifunctional compound is administered to the subject orally, parenterally, intradermally, subcutaneously, topically, or rectally.

In one embodiment, the method further comprises administering to the subject an additional therapeutic regimen for treating cancer, inflammatory disorders, or autoimmune diseases.

In one embodiment, the additional therapeutic regimen is selected from the group consisting of surgery, chemotherapy, radiation therapy, hormone therapy, targeted therapy, and immunotherapy.

In one embodiment, the TYK2-mediated diseases are selected from the group consisting of cancer, inflammatory disorders, auto-immune diseases, dermatological disorders, viral infections, dry eye disorders, bone remodeling disorders, organ transplant associated immunological complications, relapsed cancer, or the combination thereof.

In one embodiment, the TYK2-mediated cancer is selected from the group consisting of brain cancer, stomach cancer, gastrointestinal tract cancer, liver cancer, biliary passage cancer, breast cancer, ovary cancer, cervix cancer, prostate cancer, testis cancer, penile cancer, genitourinary tract cancer, esophagus cancer, larynx cancer, skin cancer, lung cancer, pancreas cancer, thyroid cancer, gland cancer, bladder cancer, kidney cancer, muscle cancer, bone cancer, cancers of the hematopoietic system, myeloproliferative neoplasms, essential thrombocythemia, polycythemia vera, primary myelofibrosis, chronic neutrophilic leukemia, acute lymphoblastic leukemia, Hodgkin's lymphoma, chronic myelomonocytic leukemia, systemic mast cell disease, hyper eosinophilic syndrome, cutaneous T-cell lymphoma, B-cell lymphoma, and myeloma.

In one embodiment, the TYK2-mediated inflammatory disorders are selected from the group consisting of ankylosing spondylitis, Crohn's disease, inflammatory bowel disease, ulcerative colitis, and ischemia reperfusion injuries.

In one embodiment, the TYK2-mediated auto-immune diseases are selected from the group consisting of multiple sclerosis, rheumatoid arthritis, psoriatic arthritis, juvenile idiopathic arthritis, psoriasis, myasthenia gravis, type I diabetes, systemic lupus erythematosus, IgA nephropathy, autoimmune thyroid disorders, alopecia areata, and bullous pemphigoid.

In one embodiment, the TYK2-mediated dermatological disorders are selected from the group consisting of atopic dermatitis, pruritus, alopecia areata, psoriasis, skin rash, skin irritation, skin sensitization, chronic mucocutaneous candidiasis, dermatomyositis, erythema multiforme, palmoplantar pustulosis, vitiligo, polyarteritis nodosa, and STING vasculopathy.

In one embodiment, the TYK2-mediated viral infections are selected from the group consisting of infections of Hepatitis B, Hepatitis C, Human Immunodeficiency Virus (HIV), Human T-lymphotropic Virus (HTLV1), Epstein Barr Virus (EBV), Varicella-Zoster Virus (VZV) and Human Papilloma Virus (HPV).

In one embodiment, the TYK2-mediated dry eye disorders are selected from the group consisting of dry eye syndrome (DES) and keratoconjunctivitis sicca (KCS).

In one embodiment, the TYK2-mediated bone remodeling disorders are selected from the group consisting of osteoporosis and osteoarthritis.

In one embodiment, the TYK2-mediated organ transplant associated immunological complications are selected from the group consisting of graft-versus-host diseases.

In one embodiment, the TYK2-mediated disease is a relapsed cancer.

In one embodiment, the TYK2-mediated disease is refractory to one or more previous treatments.

According to the 4th aspect of the present disclosure, a use of the compound according to the 1^{st} aspect of the present disclosure, or a pharmaceutically acceptable salt, or analog thereof, or the pharmaceutical composition according to the 2^{nd} aspect of the present disclosure in preparing a drug for treating and/or preventing TYK2-mediated diseases is provided.

In one embodiment, TYK2-mediated diseases are defined as before.

According to the 5th aspect of the present disclosure, a method for identifying a heterobifunctional compound which mediates degradation or reduction of TYK2 is disclosed. The method comprises:

providing a heterobifunctional test compound comprising an TYK2 ligand conjugated to a degradation tag through a linker;

contacting the heterobifunctional test compound with a cell comprising a ubiquitin ligase and TYK2;

determining whether TYK2 level is decreased in the cell; and

identifying the heterobifunctional test compound as a heterobifunctional compound which mediates degradation or reduction of TYK2.

In one embodiment, the cell is a cancer cell. In one embodiment, the cancer cell is a TYK2-mediated cancer cell.

According to the 6th aspect of the present disclosure, a method of selectively degrading or reducing TYK2 is provided comprising contacting cells with a compound of the compound according to the 1^{st} aspect of the present disclosure, or a pharmaceutically acceptable salt, or analog thereof, or the pharmaceutical composition according to the 2^{nd} aspect of the present disclosure.

In one embodiment, the cell is a cancer cell. In one embodiment, the cancer cell is a TYK2-mediated cancer cell (such as MOLT-4 cells).

In one embodiment, the method reduces TYK2 protein levels in the cells.

In one embodiment, the method is an in vitro non-therapeutic method.

According to the 7^{th} aspect of the present disclosure, a use of the heterobifunctional compound, or a pharmaceutically acceptable salt, hydrate, solvate, prodrug, stereoisomer, tautomer, or analog thereof, is provided in combination with one or more additional therapeutic agents.

In one embodiment, the heterobifunctional compound is of FORMULA I.

In one embodiment, the TYK2 ligand of the heterobifunctional compound is a moiety of FORMULAE 1 or 2 as defined as in the first aspect.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an immunoblot of TYK2, and JAK1/2/3 protein levels in MOLT-4 cells after treatment with a dose range of heterobifunctional compounds CPD-038, CPD-039, and CPD-040.

FIG. 2 shows an immunoblot of TYK2 and β-Actin proteins levels in NOMO-1 cells after treatment with a dose range of heterobifunctional compounds CPD-155, CPD-157, and CPD-158.

FIG. 3 shows an immunoblot of TYK2, STAT1/3, pY705 STAT3, pY701 STAT1 and α-tubulin levels in Jurkat cells after treatment with a dose range of heterobifunctional compounds CPD-155, CPD-158, and CPD-164, in the presence or absence of interferon α (IFNα) as indicated.

### DETAILED DESCRIPTION OF THE INVENTION

In the present disclosure, a novel approach is taken: to develop compounds that directly and selectively modulate not only the kinase activity of TYK2, but also their protein level.

Disclosed herein, in some embodiments, are heterobifunctional compounds. In some embodiments, the heterobifunctional compound comprises a chemical structure or formula disclosed herein. The heterobifunctional compound may be or include a TYK2 degrader. TYK2 degraders may be characterized by the ability to degrade or reduce cellular protein levels of TYK2. Some embodiments relate to a composition that includes the heterobifunctional compound. Some embodiments relate to methods of making the heterobifunctional compound. Some embodiments relate to methods of using the heterobifunctional compound or a pharmaceutical composition of the heterobifunctional compound. For example, the heterobifunctional compound may be used to treat a disorder or a disease. In some cases, the compound is used to treat autoimmune diseases. In some cases, the compound is used to treat inflammatory diseases. In some cases, the compound is used to treat cancers.

This disclosure includes all stereoisomers, geometric isomers, tautomers and isotopes of the structures depicted and compounds named herein. This disclosure also includes compounds described herein, regardless of how they are prepared, e.g., synthetically, through biological process (e.g., metabolism or enzyme conversion), or a combination thereof.

This disclosure includes pharmaceutically acceptable salts of the structures depicted and compounds named herein.

One or more constituent atoms of the compounds presented herein can be replaced or substituted with isotopes of the atoms in natural or non-natural abundance. In some embodiments, the compound does not include any deuterium atoms. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms. In some embodiments, the compound does not include any fluorine atoms. In some embodiments, the compound includes at least one fluorine atom. In some embodiments, the compound includes two or more fluorine atoms. In some embodiments, the compound includes 1-2, 1-3, 1-4, 1-5, or 1-6 fluorine atoms. In some embodiments, all of the hydrogen atoms in a compound can be replaced or substituted by fluorine atoms.

Heterobifunctional Compounds

Disclosed herein, in some embodiments, are compounds. In some embodiments, the compound comprises a TYK2-binding moiety disclosed herein. In some embodiments, the compound comprises a TYK2 JH2 domain-binding moiety disclosed herein. In some embodiments, the compound comprises a Degradation Tag disclosed herein. In some embodiments, the compound comprises a VHL-binding moiety. In some embodiments, the compound comprises a TYK2 degrader. For example, the compound may result in TYK2 degradation. The compound may degrade TYK2 as a result of hijacking VHL ligase function. The compound may bind to or modulate TYK2 or VHL. In some embodiments, the compound comprises a heterobifunctional compound. In some embodiments, the compound comprises a linker.

According to one aspect of the present disclosure, a heterobifunctional compound disclosed herein comprises a moiety of FORMULA I

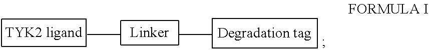

or a pharmaceutically acceptable salt, hydrate, solvate, prodrug, stereoisomer, tautomer, or analog thereof,
wherein
1) the TYK2 ligand comprises a moiety of FORMULA 1 or FORMULA 2:

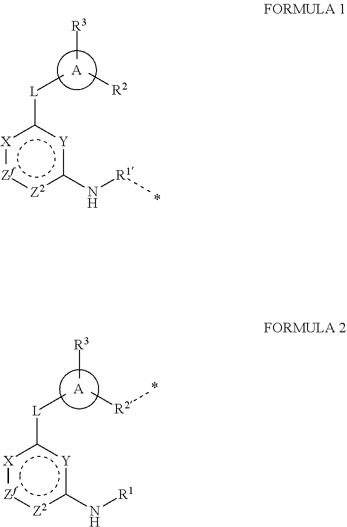

wherein
* indicates the connection to the linker moiety of the heterobifunctional compound;
L is selected from null, CR⁴R⁵, CO, CO₂, CONR⁴, NR⁴, NR⁴CO, NR⁴CO₂, NR⁴C(O)NR⁵, NR⁴SO, NR⁴SO₂, NR⁴SO₂NR⁵, O, OC(O), OCO₂, OCONR⁴, S, SO, SO₂, and SO₂NR⁴, wherein
R⁴ and R⁵ are independently selected from the group consisting of H, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkylamino, optionally substituted C₃-C₁₀ carbocyclyl, optionally substituted 3-10 membered heterocyclyl, optionally substituted aryl, and optionally substituted heteroaryl, or R⁴ and R⁵ together with the atom or atoms to which they are connected form a C₃-C₂₀ carbocyclyl ring or 3-20 membered heterocyclyl ring;
X, Y, Z¹ and Z² are independently selected from the group consisting of CR⁶ and N, wherein
R⁶, at each occurrence, is independently selected from the group consisting of null, hydrogen, halogen, CN, NO₂, OR⁷, SR⁷, NR⁷R⁸, OCOR⁷, OCO₂R⁷, OCON(R⁷)R⁸, COR⁷, CO₂R⁷, CON(R⁷)R⁸, SOR⁷, SO₂R⁷, SO₂N(R⁷)R⁸, NR⁹CO₂R⁷, NR⁹COR⁷, NR⁹C(O)N(R⁷)R⁸, NR⁹SOR⁷, NR⁹SO₂R⁷, and NR⁹SO₂N(R⁷)R⁸, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered heterocyclylC₁-C₈alkyl, optionally substituted C₃-C₁₀ carbocyclylC₁-C₈alkyl, optionally substituted C₃-C₁₀ carbocyclyl, optionally substituted 3-10 membered heterocyclyl, optionally substituted aryl, and optionally substituted heteroaryl; or two R⁶ groups together with the atoms to which they are connected optionally form optionally substituted C₅-C₆ carbocyclyl, optionally substituted 5-6 membered heterocyclyl, optionally substituted C₆ aryl, and optionally substituted 5-6 membered heteroaryl, wherein
R⁷, R⁸, and R⁹ are independently selected from the group consisting of null, hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted C₃-C₁₀ carbocyclylC₁-C₈alkyl, optionally substituted 3-10 membered heterocyclylC₁-C₈alkyl, optionally substituted C₃-C₁₀ carbocyclyl, optionally substituted 3-10 membered heterocyclyl, optionally substituted aryl, and optionally substituted heteroaryl, or R⁷ and R⁸, R⁷ and R⁹ together with the atom to which they are connected form a 3-20 membered heterocyclyl ring;
R² (in FORMULA 1) and R¹ (in FORMULA 2) are selected from the group consisting of null, hydrogen, halogen, CN, NO₂, OR¹⁰, SR¹⁰, NR¹⁰R¹¹, OCOR¹⁰, OCO₂R¹⁰, OCONR¹⁰R¹¹, COR¹⁰, CO₂R¹⁰, CONR¹⁰R¹¹, SOR¹⁰, SO₂R¹⁰, SO₂NR¹⁰R¹¹, NR¹²CO₂R¹⁰, NR¹²COR¹⁰, NR¹²C(O)NR¹⁰R¹¹, NR¹²SOR¹⁰, NR¹²SO₂R¹⁰, NR¹²SO₂NR¹⁰R¹¹, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylamino, optionally substituted C₃-C₈ cycloalkylamino, optionally substituted C₁-C₈alkylaminoC₁-C₈alkyl, optionally substituted C₃-C₁₀ carbocyclyl, optionally substituted C₃-C₁₀ cycloalkoxy, optionally substituted C₃-C₁₀ carbocyclylamino, optionally substituted 3-10 membered heterocyclyl, optionally substituted aryl, and optionally substituted heteroaryl, wherein
   R¹⁰, R¹¹, and R¹² are independently selected from the group consisting of hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted C₃-C₁₀ carbocyclyl, optionally substituted 3-10 membered heterocyclyl, optionally substituted aryl, and optionally substituted heteroaryl; or R¹⁰ and R¹¹, or R¹⁰ and R¹² together with the atom(s) to which they are connected form a C₃-C₂₀ carbocyclyl or 3-20 membered heterocyclyl ring;
R^{1′} (in FORMULA 1) and R^{2′} (in FORMULA 2) are divalent groups independently selected from the group consisting of null, R′—R″, R′OR″, R′SR″, R′N(R¹³)R″, R′OC(O)R″, R′OC(O)OR″, R′OCON(R¹³)R″, R′C(O)R″, R′C(O)OR″, R′CON(R¹³)R″, R′S(O)R″, R′S(O)₂R″, R′SO₂N(R¹³)R″, R′N(R¹⁴)C(O)OR″, R′N(R¹⁴)C(O)R″, R′N(R¹⁴)C(O)N(R¹³)R″, R′N(R¹⁴)S(O)R″, R′N(R¹⁴)S(O)₂R″, and R′N(R¹⁴)S(O)₂N(R¹³)R″, optionally substituted C₃-C₁₃ carbocyclyl, optionally substituted 3-13 membered heterocyclyl, optionally substituted aryl, and optionally substituted heteroaryl, wherein
R′ and R″ are divalent groups independently selected from the group consisting of null, optionally substituted C₁-C₈ alkylene, optionally substituted C₂-C₈ alkenylene, optionally substituted C₂-C₈ alkynylene, optionally substituted C₃-C₁₀ carbocyclyl, optionally substituted 3-10 membered heterocyclyl, optionally substituted aryl, and optionally substituted heteroaryl;
R¹³ and R¹⁴ are independently selected from the group consisting of H, optionally substituted C₁-C₈ alkyl, optionally substituted C₂-C₈ alkenyl, optionally substituted C₂-C₈ alkynyl, optionally substituted C₃-C₁₀ carbocyclyl, optionally substituted 3-10 membered heterocyclyl, optionally substituted aryl, and optionally substituted heteroaryl; or
R′ and R″, R¹³ and R¹⁴, R′ and R¹³, R′ and R¹⁴, R″ and R¹³ or R″ and R¹⁴, together with the atom to which they are connected, form a C₃-C₂₀ carbocyclyl or 3-20 membered heterocyclyl ring;
Ring A

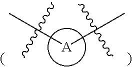

is selected from the group consisting of optionally substituted C₃-C₁₃ carbocyclyl, optionally substituted 3-13 membered heterocyclyl, optionally substituted aryl, and optionally substituted heteroaryl;
R³ means one or more substituents which are selected from the group consisting of null, hydrogen, halogen, CN, NO₂, OR¹⁵, SR¹⁵, NR¹⁵R¹⁶, OCOR¹⁵, OCO₂R¹⁵, OCON(R¹⁵)R¹⁶, COR¹⁵, CO₂R¹⁵, CON(R¹⁵)R¹⁶, SOR¹⁵, SO₂R¹⁵, SO₂N(R¹⁵)R¹⁶, NR¹⁷CO₂R¹⁵, NR¹⁷COR¹⁵, NR¹⁷C(O)N(R¹⁵)R¹⁶, NR¹⁷SOR¹⁵, NR¹⁷SO₂R¹⁵, NR⁷SO₂N(R¹⁵)R¹⁶, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered heterocyclylC₁-C₈alkyl, optionally substituted C₃-C₁₀ carbocyclylC₁-C₈alkyl, optionally substituted C₃-C₁₀ carbocyclyl, optionally substituted 3-10 membered heterocyclyl, optionally substituted aryl, and optionally substituted heteroaryl; or two R³ groups together with the atoms to which they are connected optionally form optionally substituted C₅-C₆ carbocyclyl, optionally substituted 5-6 membered heterocyclyl, optionally substituted C₆ aryl, and optionally substituted 5-6 membered heteroaryl, wherein
R¹⁵, R¹⁶, and R¹⁷ are independently selected from the group consisting of null, hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted C₃-C₁₀ carbocyclylC₁-C₈alkyl, optionally substituted 3-10 membered heterocyclylC₁-C₈alkyl, optionally substituted C₃-C₁₀ carbocyclyl, optionally substituted 3-10 membered heterocyclyl, optionally substituted aryl, and optionally substituted heteroaryl, or R¹⁵ and R¹⁶, R¹⁵ and R¹⁷ together with the atom to which they are connected form a 3-20 membered heterocyclyl ring;
2) the degradation tag is a moiety of FORMULA 6A, 6B, or 6C:

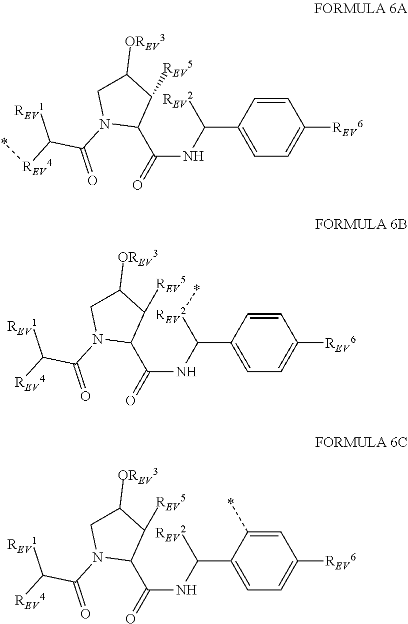

wherein
* indicates the connection to the linker moiety of the heterobifunctional compound;
R_{EV}¹ and R_{EV}² are independently selected from the group consisting of hydrogen, hydroxyl, amino, cyano, nitro, optionally substituted C₁-C₈ alkyl, optionally substituted C₂-C₈ alkenyl, optionally substituted C₂-C₈ alkynyl; optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted C₃-C₁₀ carbocyclyl, optionally substituted 3-10 membered heterocyclyl;
R_{EV}^{2′} is a divalent group selected from the group consisting of null, O, NH, optionally substituted C₁-C₈ alkylene, optionally substituted C₂-C₈ alkenylene, optionally substituted C₂-C₈ alkynylene; optionally substituted C₁-C₈alkoxyC₁-C₈alkylene, optionally substituted C₁-C₈ haloalkylene, optionally substituted C₁-C₈ hydroxyalkylene, optionally substituted C₁-C₈ aminoalkylene, optionally substituted C₁-C₈alkylaminoC₁-C₈alkylene, optionally substituted C₃-C₁₀ carbocyclyl, and optionally substituted 3-10 membered heterocyclyl;
R_{EV}³ is selected from the group consisting of hydrogen, optionally substituted —C(O)R_{EV}⁷, —C(O)OR_{EV}⁷, —C(O)NR_{EV}⁷R_{EV}⁸, —P(O)(OR_{EV}⁷)₂, and —CR_{EV}⁷R_{EV}⁸—OP(O)(OR_{EV}⁹)₂, wherein
R_{EV}⁷, R_{EV}⁸ and R_{EV}⁹ are independently selected from the group consisting of hydrogen, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted C₃-C₁₀ carbocyclyl, optionally substituted 3-10 membered heterocyclyl, optionally substituted C₂-C₈ alkenyl, optionally substituted C₂-C₈ alkynyl, optionally substituted aryl and optionally substituted heteroaryl;
R_{EV}⁴ is selected from the group consisting of —N(R_{EV}¹⁰)R_{EV}¹¹, —OR_{EV}¹⁰, —N(R_{EV}¹⁰)C(O)R_{EV}¹¹, optionally substituted C₃-C₁₀ carbocyclyl, optionally substituted 3-10 membered heterocyclyl, optionally substituted aryl, and optionally substituted heteroaryl;
R_{EV}^{4′} is a divalent group selected from the group consisting of —N(R_{EV}¹⁰)—, —O—, —N(R_{EV}¹⁰)C(O)R_{EV}^{11′}—, optionally substituted C₃-C₁₀ carbocyclyl, optionally substituted 3-10 membered heterocyclyl, optionally substituted aryl, and optionally substituted heteroaryl;
R_{EV}¹⁰ is selected from the group consisting of hydrogen, optionally substituted C₁-C₈alkyl, optionally substituted C₃-C₈cycloalkyl, optionally substituted C₁-C₈alkyl-CO, optionally substituted C₁-C₈cycloalkyl-CO, optionally substituted C₃-C₈cycloalkyl-C₁-C₈alkyl-CO, optionally substituted 3-10 membered heterocyclyl-CO, optionally substituted 3-10 membered heterocyclyl-C₁-C₈alkyl-CO, optionally substituted aryl-CO, optionally substituted aryl-C₁-C₈alkyl-CO, optionally substituted heteroaryl-CO, optionally substituted heteroaryl-C₁-C₈alkyl-CO, optionally substituted aryl, and optionally substituted heteroaryl;
R_{EV}¹¹ is selected from the group consisting of hydrogen, optionally substituted C₁-C₈alkyl, optionally substituted C₃-C₈cycloalkyl, optionally substituted 3-8 membered heterocycloalkyl, optionally substituted C₃-C₈ carbocyclclyl, and optionally substituted C₃-C₈ heterocyclclyl;
R_{EV}^{11′}, at each occurrence, is a divalent group independently selected from the group consisting of null, O, optionally substituted C₁-C₈alkylene, optionally substituted C₃-C₈ cycloalkylene, optionally substituted 3-8 membered heterocycloalkylene, optionally substituted C₃-C₈ carbocyclclyl, and optionally substituted C₃-C₈ heterocyclclyl;
R_{EV}⁵ is selected from the group consisting of hydrogen and halogen (such as F); and
R_{EV}⁶ is selected from hydrogen, halogen, hydroxyl, amino, cyano, nitro, optionally substituted C₁-C₈alkyl, optionally substituted C₁-C₈cycloalkyl, optionally substituted C₁-C₈alkoxy, and optionally substituted C₁-C₈cycloalkoxy, optionally substituted C₁-C₈heterocycloalkyl, optionally substituted aryl, and optionally substituted heteroaryl;
or, the degradation tag is a moiety of FORMULA 5, and the degradation tag is connected to the linker moiety of the heterobifunctional compound via Z_{E};

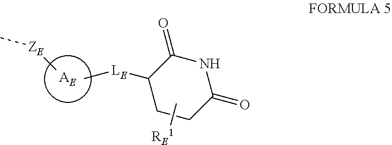

wherein Ring A_{E}, L_{E}, Z_{E}, and R_{E}¹ are defined as hereinafter;
and 3) the linker moiety is of FORMULA 9:

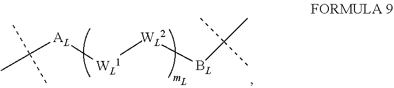

wherein
A_{L}, W_{L}¹, W_{L}², and B_{L}, at each occurrence, are bivalent moieties independently selected from the group consisting of null, R_{L}^{d}—R_{L}^{e}, R_{L}^{d}COR_{L}^{e}, R_{L}^{d}C(O)OR_{L}^{e}, R_{L}^{d}C(O)N(R_{L}¹)R_{L}^{e}, R_{L}^{d}C(S)N(R_{L}¹)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}¹)R_{L}^{e}, R_{L}^{d}N(R_{L}¹)R_{L}^{e}, R_{L}^{d}N(R_{L}¹)COR_{L}^{e}, R_{L}^{d}N(R_{L}¹)CON(R_{L}²)R_{L}^{e}, R_{L}^{d}N(R_{L}¹)C(S)R_{L}^{e}, optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₁-C₈alkoxyC₁-C₈alkylene, optionally substituted C₁-C₈ haloalkylene, optionally substituted C₁-C₅ hydroxyalkylene, optionally substituted C₃-C₁₃ cycloalkyl, optionally substituted 3-13 membered heterocyclyl, optionally substituted aryl, and optionally substituted heteroaryl (preferably, A_{L}, W_{L}¹, W_{L}², and B_{L}, at each occurrence, are bivalent moieties independently selected from the group consisting of null, R_{L}^{d}—R_{L}^{e}, R_{L}^{d}COR_{L}^{e}, R_{L}^{d}C(O)OR_{L}^{e}, R_{L}^{d}C(O)N(R_{L}¹)R_{L}^{e}, R_{L}^{d}C(S)N(R_{L}¹)R_{L}^{e}, R_{L}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}¹)R_{L}^{e}, R_{L}^{d}N(R_{L}¹)R_{L}^{e}, R_{L}^{d}N(R_{L}¹)COR_{L}^{e}, R_{L}^{d}N(R_{L}¹)CON(R_{L}²)R_{L}^{e}, R_{L}^{d}N(R_{L}¹)C(S)R_{L}^{e}, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ cycloalkyl, optionally substituted 3-13 membered heterocyclyl, optionally substituted aryl, and optionally substituted heteroaryl); wherein
R_{L}^{d} and R_{L}^{e}, at each occurrence, are independently selected from null, R_{L}^{r}, optionally substituted (C₁-C₈ alkylene)-R_{L}^{r} (preferably, CH₂—R_{L}^{r}), optionally substituted R_{L}^{r}—(C₁-C₈ alkylene), optionally substituted (C₁-C₈ alkylene)-R_{L}^{r}-(C₁-C₈ alkylene), or a bivalent moiety comprising of optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted C₃-C₁₃ cycloalkyl, optionally substituted 3-13 membered heterocyclyl, optionally substituted aryl, and optionally substituted heteroaryl (preferably, R_{L}^{d} and R_{L}^{e}, at each occurrence, are independently selected from null, R_{L}^{r}, optionally substituted (C₁-C₈ alkylene)-R_{L}^{r} (preferably, CH₂—R_{L}^{r}), optionally substituted R_{L}^{r}-(C₁-C₈ alkylene), optionally substituted (C₁-C₈ alkylene)-R_{L}^{r}-(C₁-C₈ alkylene), or a bivalent moiety comprising of optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted C₃-C₁₃ cycloalkyl, optionally substituted 3-13 membered heterocyclyl, optionally substituted aryl, and optionally substituted heteroaryl);
R_{L}^{r}, at each occurrence, is selected from optionally substituted C₃-C₁₀ carbocyclyl, optionally substituted 3-10 membered heterocyclyl, optionally substituted aryl, and optionally substituted heteroaryl;
R_{L}¹ and R_{L}², at each occurrence, are independently selected from the group consisting of hydrogen, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₀ carbocyclyl, optionally substituted 3-10 membered heterocyclyl, optionally substituted aryl, and optionally substituted heteroaryl (preferably, R_{L}¹ and R_{L}², at each occurrence, are independently selected from the group consisting of hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₀ carbocyclyl, optionally substituted 3-10 membered heterocyclyl, optionally substituted aryl, and optionally substituted heteroaryl);
R_{L}^{d} and R_{L}^{e}, R_{L}¹ and R_{L}², R_{L}^{d} and R_{L}¹, R_{L}^{d} and R_{L}², R_{L}^{e} and R_{L}¹, or R_{L}^{e} and R_{L}² together with the atom(s) to which they are connected optionally form a C₃-C₂₀ carbocyclyl or 3-20 membered heterocyclyl ring; and
m_{L} is 1, 2, 3, 4, 5, 6, 7, 8, 9, 10, 11, 12, 13, 14, or 15.

In some embodiments, the cycloalkyl includes monocyclic carbocyclyl, fused cycloalkyl, bridged cycloalkyl, or spiro cycloalkyl.

In some embodiments, the carbocyclyl includes monocyclic carbocyclyl, fused carbocyclyl, spiro carbocyclyl, or bridged carbocyclyl.

In some embodiments, the heterocyclyl includes monocyclic heterocyclyl, bridged heterocyclyl, fused heterocyclyl, or spiro heterocyclyl.

In some embodiments, the aryl includes monocyclic aryl, bicyclic fused aryl, or tricyclic fused aryl.

In some embodiments, the heteroaryl includes monocyclic heteroaryl, bicyclic fused heteroaryl, or tricyclic fused heteroaryl.

In some embodiments, each C₃-C₁₃ cycloalkyl, at each occurrence, is independently selected from C₃-C₁₀ monocyclic carbocyclyl, C₄-C₁₃ fused cycloalkyl, C₅-C₁₃ bridged cycloalkyl, or C₅-C₁₃ spiro cycloalkyl.

In some embodiments, the C₃-C₁₃ carbocyclyl, at each occurrence, is independently selected from C₃-C₁₀ monocyclic carbocyclyl, C₄-C₁₃ fused carbocyclyl, C₅-C₁₃ spiro carbocyclyl, or C₅-C₁₃ bridged carbocyclyl.

In some embodiments, the 3-13 membered heterocyclyl, at each occurrence, is independently selected from 3-10 membered monocyclic heterocyclyl, 5-13 membered bridged heterocyclyl, 5-13 membered fused heterocyclyl, or 5-13 membered spiro heterocyclyl.

In some embodiments, the aryl, at each occurrence, is independently selected from monocyclic aryl, bicyclic fused aryl, or tricyclic fused aryl.

In some embodiments, the heteroaryl, at each occurrence, is independently selected from monocyclic heteroaryl, bicyclic fused heteroaryl, or tricyclic fused heteroaryl.

In some preferred embodiments, the TYK2 ligand is a moiety of FORMULA 1.

In some embodiments, the TYK2 ligand is a moiety of FORMULA 1-1, 1-2, 2-1, or 2-2:

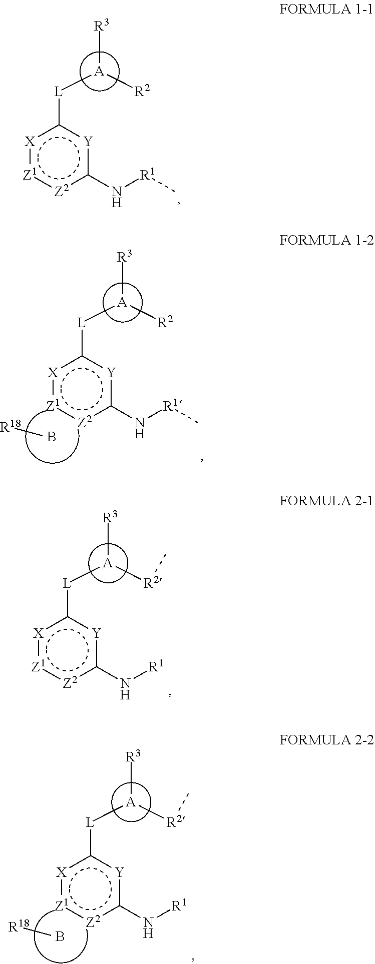

wherein

X, Y, Z¹, and Z² are independently selected from the group consisting of CR⁶ and N, wherein

R⁶, at each occurrence, is independently selected from the group consisting of null, hydrogen, halogen, CN, NO₂, OR⁷, SR⁷, NR⁷R⁸, OCOR⁷, OCO₂R⁷, OCON(R⁷)R⁸, COR⁷, CO₂R⁷, CON(R⁷)R⁸, SOR⁷, SO₂R⁷, SO₂N(R⁷)R⁸, NR⁹CO₂R⁷, NR⁹COR⁷, NR⁹C(O)N(R⁷)R⁸, NR⁹SOR⁷, NR⁹SO₂R⁷, NR⁹SO₂N(R⁷)R⁸, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered heterocyclylC₁-C₈alkyl, optionally substituted C₃-C₁₀ carbocyclylC₁-C₈alkyl, optionally substituted C₃-C₁₀ carbocyclyl, optionally substituted 3-10 membered heterocyclyl, optionally substituted aryl, and optionally substituted heteroaryl;

L, Ring A, R¹, R², R^{1′}, R^{2′}, R³, R⁷, R⁸ and R⁹ are defined as in FORMULAE 1 or 2;

Ring B is selected from optionally substituted 5-6 membered carbocyclyl, optionally substituted 5-6 membered heterocyclyl, optionally substituted aryl, optionally substituted heteroaryl; and

R¹⁸ means one or more groups which are independently selected from the group consisting of null, hydrogen, halogen, CN, NO₂, OR¹⁹, SR¹⁹, NR¹⁹R²⁰, OCOR¹⁹, OCO₂R¹⁹, OCON(R¹⁹)R²⁰, COR¹⁹, CO₂R¹⁹, CON(R¹⁹)R²⁰, SOR¹⁹, SO₂R¹⁹, SO₂N(R¹⁹)R²⁰, NR²¹CO₂R¹⁹, NR²¹COR¹⁹, NR²¹C(O)N(R¹⁹)R²⁰, NR²¹SOR¹⁹, NR²¹SO₂R¹⁹, NR²¹SO₂N(R¹⁹)R²⁰, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered heterocyclylC₁-C₈alkyl, optionally substituted C₃-C₁₀ carbocyclylC₁-C₈ alkyl, optionally substituted C₃-C₁₀ carbocyclyl, optionally substituted 3-10 membered heterocyclyl, optionally substituted aryl, and optionally substituted heteroaryl; or two R⁵ groups together with the atoms to which they are connected optionally form optionally substituted 5-6 membered carbocyclyl, optionally substituted 5-6 membered heterocyclyl, optionally substituted C₆ aryl, and optionally substituted 5-6 membered heteroaryl, wherein

R¹⁹, R²⁰, and R²¹ are independently selected from null, hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted C₃-C₁₀ carbocyclylC₁-C₈alkyl, optionally substituted 3-10 membered heterocyclylC₁-C₈alkyl, optionally substituted C₃-C₁₀ carbocyclyl, optionally substituted 3-10 membered heterocyclyl, optionally substituted aryl, and optionally substituted heteroaryl, or R¹⁹ and R²⁰, R¹⁹ and R²¹ together with the atom to which they are connected form a 3-20 membered heterocyclyl ring.

In some embodiments, the TYK2 ligand is a moiety of FORMULA 1-1.

In some embodiments, the TYK2 ligand is a moiety of FORMULA 1-1A, 1-1B, 1-2A, 1-2B, 1-2C, 2-1A, 2-1B, 2-2A, 2-2B, or 2-2C:

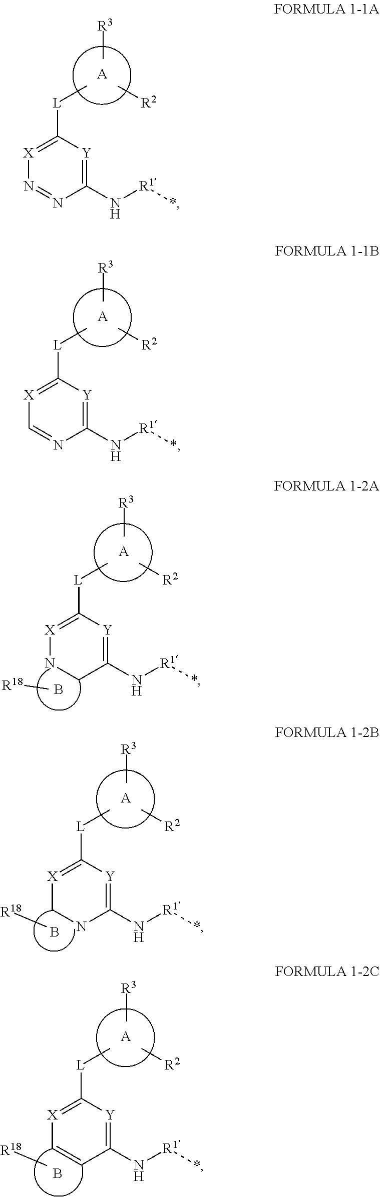

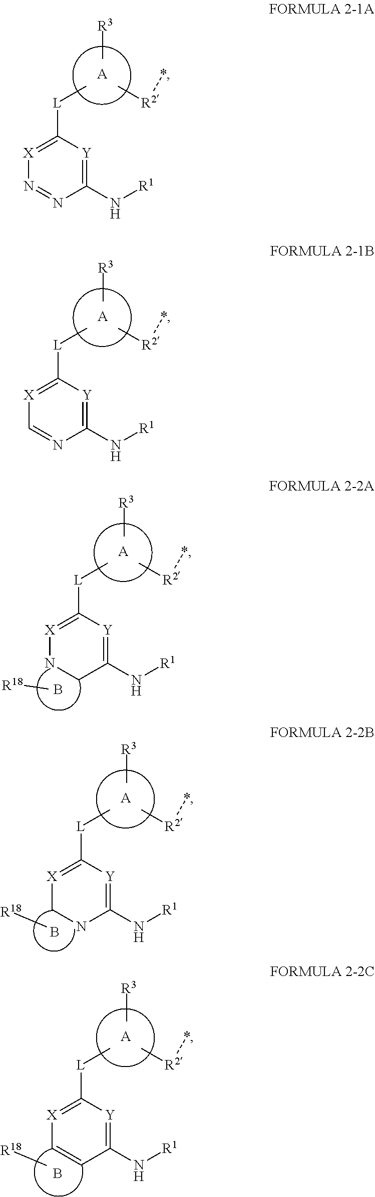

wherein L, Ring A, R¹, R², R^{1′}, R^{2′}, and R³ are defined as in FORMULAE 1 and 2; and Ring B, X, Y, and R¹⁸ are defined as in FORMULAE 1-1, 1-2, 2-1, and 2-2.

In some embodiments, the TYK2 ligand is a moiety of FORMULA 1-1A.

In some embodiments, Ring B is selected from optionally substituted 5-6 membered heterocyclyl, and optionally substituted 5-6 membered heteroaryl.

In some embodiments, Ring B is selected from optionally substituted 5 membered heteroaryl.

In some embodiments, the TYK2 ligand is a moiety of FORMULA 1-2D, 1-2E, 1-2F, 2-2D, 2-2E, or 2-2F:

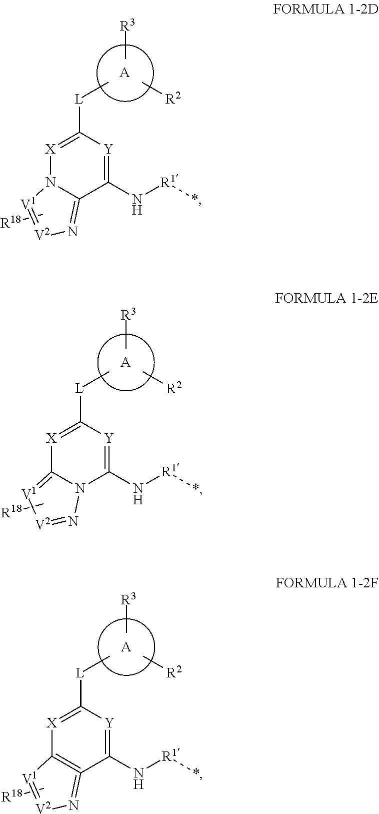

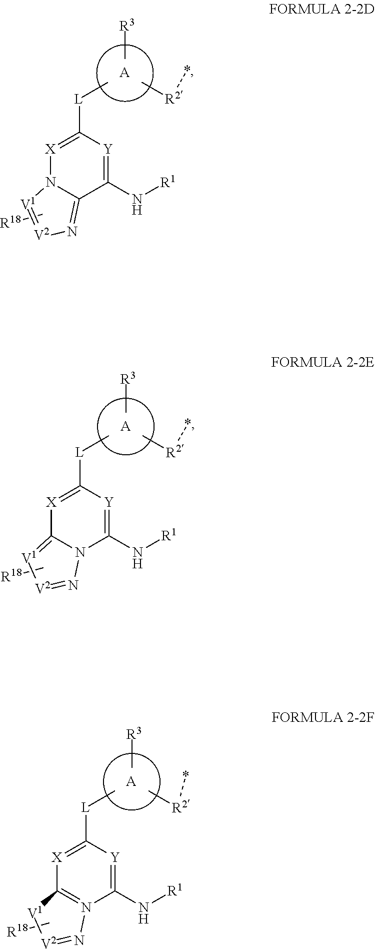

wherein

L, Ring A, R¹, R², R^{1′}, R^{2′}, and R³ are defined as in FORMULAE 1 and 2;

V¹ and V² are independently selected from CH and N; and

X, Y, R¹⁸ is defined as in FORMULAE 1-1, 1-2, 2-1, or 2-2.

In some embodiments, the TYK2 ligand is a moiety of FORMULA 1-2G or 2-2G:

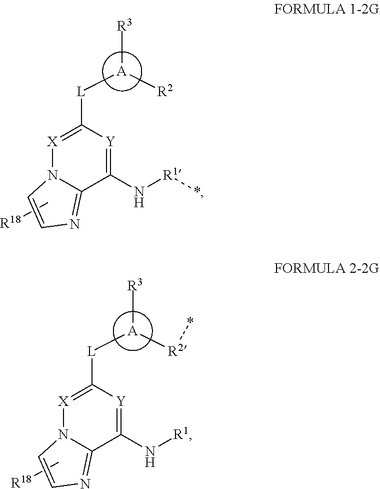

wherein

L, Ring A, R¹, R², R^{1′}, R^{2′}, and R³ are defined as in FORMULAE 1 and 2;

X, Y, and R¹⁸ is defined as in FORMULAE 1-1, 1-2, 2-1, and 2-2.

In some embodiments, the TYK2 ligand is a moiety of FORMULA 1-1C, 1-1D, 1-2H, 2-1C, 2-1D, or 2-2H:

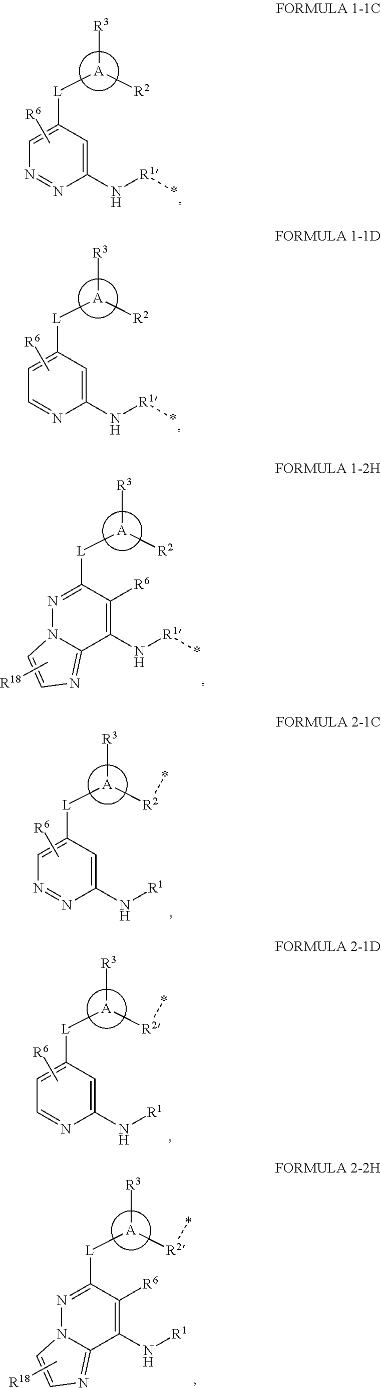

wherein

L, Ring A, R¹, R², R^{1′}, R^{2′}, and R³ are defined as in FORMULAE 1 and 2; and

R⁶ and R¹⁸ are defined as in FORMULAE 1-1, 1-2, 2-1, and 2-2.

In some preferred embodiments, the TYK2 ligand is a moiety of FORMULA 1-1C.

In some embodiments, Ring A is selected from optionally substituted 5-6 membered carbocyclyl, optionally substituted 5-6 membered heterocyclyl, optionally substituted C₆ aryl and optionally substituted 5-6 membered heteroaryl.

In some embodiments, Ring A is selected from optionally substituted phenyl or pyridinone.

In some embodiments, the TYK2 ligand is a moiety of FORMULA 1-1E, 1-1F, 1-2I, 2-1E, 2-1F or 2-2I:

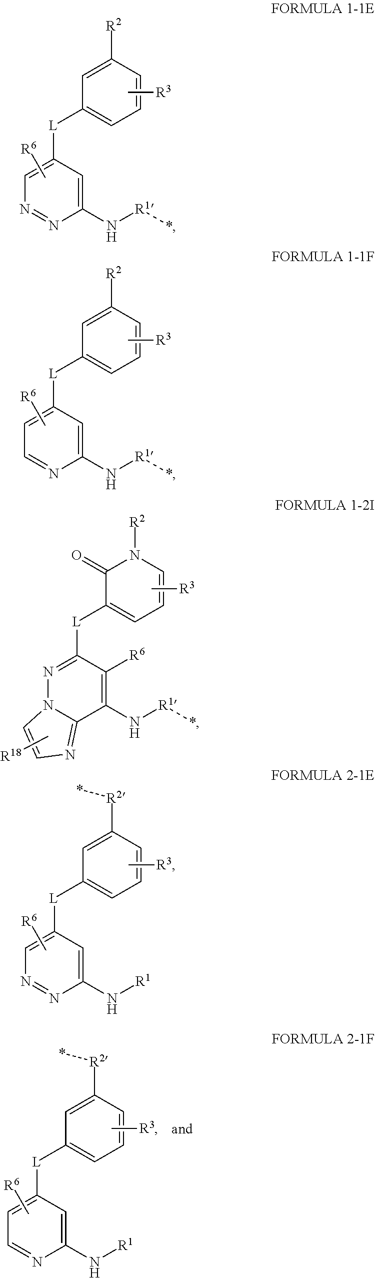

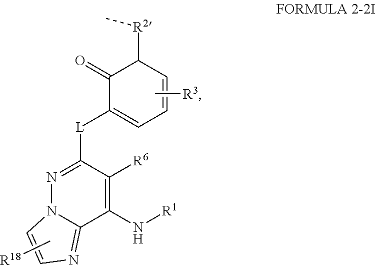

wherein

L, R¹, R², R^{1′}, R^{2′}, and R³ are defined as in FORMULAE 1 and 2; and

R⁶ and R¹⁸ is defined as in FORMULAE 1-1, 1-2, 2-1, and 2-2.

In some preferred embodiments, the TYK2 ligand is a moiety of FORMULA 1-1E.

In some embodiments, L is selected from CR⁴R⁵, NR⁴, and O.

In some embodiments, R⁴ and R⁵ are independently selected from H, halogen, hydroxyl, amino, cyano, nitro, optionally substituted C₁-C₆ alkyl, and optionally substituted C₃-C₆ cycloalkyl. In some embodiments, R⁴ and R⁵ are independently selected from H, halogen, optionally substituted C₁-C₆ alkyl, and optionally substituted C₃-C₆ cycloalkyl. In some embodiments, R⁴ and R⁵ are independently selected from H, F, Me, Et. iPr, and cPr.

In some embodiments, L is selected from NH and N(CH₃). In some embodiments, L is NH.

In some embodiments, the TYK2 ligand is a moiety of FORMULA 1-1G, 1-1H, 1-2J, 2-1G, 2-1H or 2-2J:

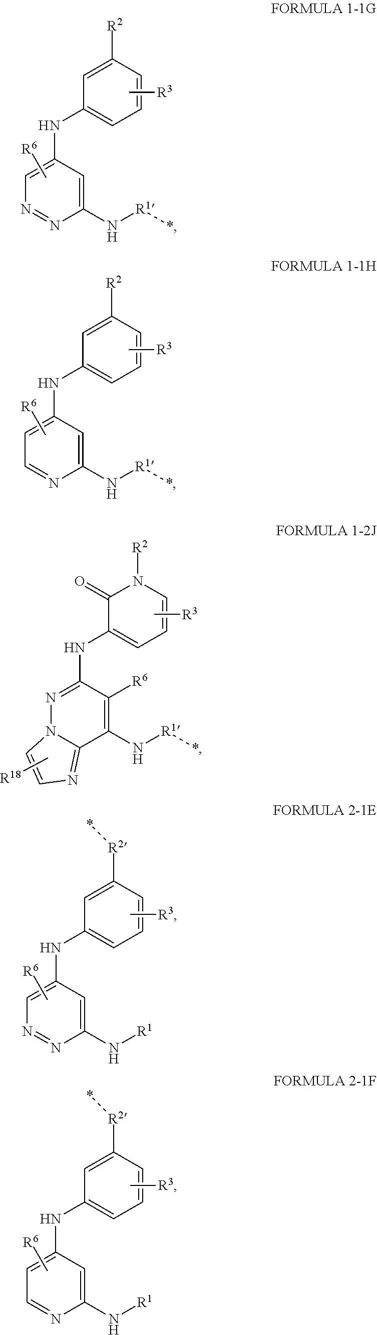

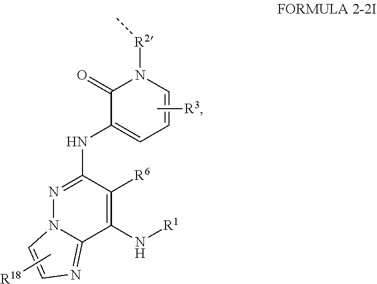

wherein

R¹, R², R^{1′}, R^{2′}, and R³ are defined as in FORMULAE 1 and 2; and

R⁶ and R¹⁸ is defined as in FORMULAE 1-1, 1-2, 2-1, and 2-2.

In some preferred embodiments, the TYK2 ligand is a moiety of FORMULA 1-1G.

In some embodiments, R⁶, at each occurrence, is independently selected from the group consisting of hydrogen, halogen, CN, NO₂, COR⁷, CON(R⁷)R⁸, optionally substituted C₁-C₈ alkyl, optionally substituted C₂-C₈ alkenyl, optionally substituted C₃-C₁₀ carbocyclyl, optionally substituted 3-10 membered heterocyclyl, wherein

R⁷ and R⁸ are independently selected from the group consisting of null, hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted C₃-C₁₀ carbocyclylC₁-C₈alkyl, optionally substituted 3-10 membered heterocyclylC₁-C₈alkyl, optionally substituted C₃-C₁₀ carbocyclyl, optionally substituted 3-10 membered heterocyclyl, optionally substituted aryl, and optionally substituted heteroaryl, or R⁷ and R⁸ together with the atom to which they are connected form a 3-20 membered heterocyclyl ring.

In some embodiments, the TYK2 ligand is a moiety of FORMULA 1-1I, 1-1J, 1-2K, 2-1I, 2-1J or 2-2K:

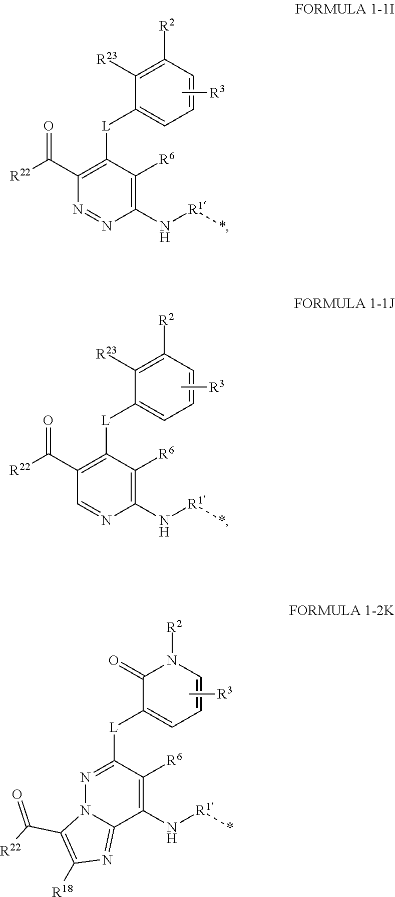

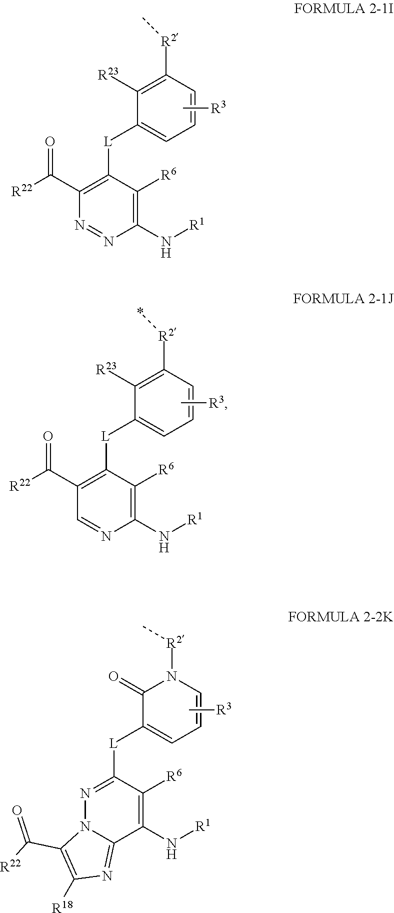

wherein

R²² is R⁷ or NHR⁷;

R²³ is defined as R³;

L, R¹, R², R^{1′}, R^{2′}, R³, R⁷ and R⁸ are defined as in FORMULAE 1 and 2; and R⁶ is defined as in FORMULAE 1-1, 1-2, 2-1, and 2-2.

In some embodiments, the TYK2 ligand comprises FORMULAE 1-1I and 2-1I.

In some embodiments, the TYK2 ligand comprises FORMULA 1-1I.

In some embodiments, R¹ and R² are independently selected from the group consisting of null, hydrogen, halogen, CN, NO₂, OR¹⁰, NR¹⁰R¹¹, COR¹⁰, CONR¹⁰R¹¹, optionally substituted C₁-C₈ alkyl, optionally substituted C₂-C₈ alkenyl, optionally substituted C₂-C₈ alkynyl, optionally substituted C₃-C₁₀ carbocyclyl, optionally substituted 3-10 membered heterocyclyl, optionally substituted aryl, and optionally substituted heteroaryl, wherein

R¹⁰ and R¹¹ are independently selected from the group consisting of null, hydrogen, optionally substituted C₁-C₈ alkyl, optionally substituted C₂-C₈ alkenyl, optionally substituted C₂-C₈ alkynyl, optionally substituted C₃-C₁₀ carbocyclyl, optionally substituted 3-10 membered heterocyclyl, optionally substituted aryl, and optionally substituted heteroaryl, or R¹⁰ and R¹¹, together with the atom(s) to which they are connected optionally form a 3-20 membered heterocyclyl ring.

In some embodiments, R¹ is selected from COR¹⁰, optionally substituted C₁-C₈ alkyl, optionally substituted C₃-C₁₀ carbocyclyl, optionally substituted 3-10 membered heterocyclyl, optionally substituted aryl, and optionally substituted heteroaryl, wherein R¹⁰ is selected from null, hydrogen, optionally substituted C₁-C₈ alkyl, optionally substituted C₃-C₁₀ carbocyclyl, optionally substituted 3-10 membered heterocyclyl, optionally substituted aryl, and optionally substituted heteroaryl.

In some embodiments, R¹ is selected from optionally substituted C(O)-cPr, optionally substituted methyl, optionally substituted pyridinyl, optionally substituted phenyl, optionally substituted pyrazinyl, optionally substituted pyrimidinyl, optionally substituted pyridazinyl, optionally substituted triazinyl, optionally substituted pyrrolyl, optionally substituted furanyl, optionally substituted thiophenyl, optionally substituted imidazolyl, optionally substituted pyrazolyl, optionally substituted oxazolyl, optionally substituted isoxazolyl, optionally substituted thiazolyl, optionally substituted isothiazolyl, optionally substituted triazolyl, optionally substituted oxadiazolyl, optionally substituted thiadiazolyl, and optionally substituted tetrazolyl.

In some embodiments, R¹ is selected from optionally substituted C(O)-cPr, optionally substituted pyridinyl and optionally substituted methyl.

In some embodiments, R² is selected from H, CN, halogen, CO₂R¹⁰, CONR¹⁰R¹¹, optionally substituted aryl, and optionally substituted heteroaryl. In some embodiments, R² is selected from optionally substituted aryl, and optionally substituted heteroaryl.

In some embodiments, R² is selected from the group consisting of H, CN, F, Cl, Br, CO₂H, CONH₂, CONHCH₃, optionally substituted triazolyl, optionally substituted phenyl, optionally substituted pyridinyl, optionally substituted pyrazinyl, optionally substituted pyrimidinyl, optionally substituted pyridazinyl, triazinyl, optionally substituted pyrrolyl, furanyl, optionally substituted thiophenyl, optionally substituted imidazolyl, optionally substituted pyrazolyl, optionally substituted oxazolyl, optionally substituted isoxazolyl, optionally substituted thiazolyl, optionally substituted isothiazolyl, optionally substituted oxadiazolyl, optionally substituted thiadiazolyl, and optionally substituted tetrazolyl.

In some embodiments, R² is selected from the group consisting of optionally substituted triazolyl, optionally substituted phenyl, optionally substituted pyridinyl, optionally substituted pyrazinyl, optionally substituted pyrimidinyl, optionally substituted pyridazinyl, triazinyl, optionally substituted pyrrolyl, furanyl, optionally substituted thiophenyl, optionally substituted imidazolyl, optionally substituted pyrazolyl, optionally substituted oxazolyl, optionally substituted isoxazolyl, optionally substituted thiazolyl, optionally substituted isothiazolyl, optionally substituted oxadiazolyl, optionally substituted thiadiazolyl, and optionally substituted tetrazolyl.

In some embodiments, R² is selected from H, CN, F, Cl, Br, CO₂H, CONH₂, CONHCH₃, optionally substituted triazolyl and optionally substituted phenyl. In some embodiments, R² is selected from, optionally substituted triazolyl and optionally substituted phenyl.

In some embodiments, the substituent(s) for R² are independently optionally substituted groups selected from CN, F, Cl, Br, C₁-C₈ alkyl (such as C₁-C₄ alkyl), C₃-C₈ carbocyclyl (such as cyclopropyl), and C₁-C₈ haloalkyl (such as C₁-C₄ haloalkyl).

In some embodiments, R^{1′} and R^{2′} are selected from the group consisting of are selected from the group consisting of null, R′—R″, R′OR″, R′SR″, R′N(R¹³)R″, R′OC(O)R″, R′OC(O)OR″, R′OCON(R¹³)R″, R′C(O)R″, R′C(O)OR″, R′CON(R¹³)R″, R′S(O)R″, R′S(O)₂R″, R′SO₂N(R¹³)R″, R′NR¹⁴C(O)OR″, R′NR¹⁴C(O)R″, R′NR¹⁴C(O)N(R¹³)R″, R′NR¹⁴S(O)R″, R′NR¹⁴S(O)₂R″, and R′NR¹⁴S(O)₂NR¹³R″, optionally substituted C₁-C₈ alkylene, optionally substituted C₃-C₁₃ carbocyclyl, optionally substituted 3-13 membered heterocyclyl, optionally substituted aryl, and optionally substituted heteroaryl, wherein R′ and R″ are divalent groups independently selected from the group consisting of null, optionally substituted C₁-C₈ alkylene (such as CH₂).

In some embodiments, R^{1′} (in FORMULA 1) is a divalent group selected from the group consisting of null, R′—R″, R′C(O)R″, optionally substituted C₃-C₁₃ carbocyclyl, optionally substituted 3-13 membered heterocyclyl, optionally substituted aryl, and optionally substituted heteroaryl; and wherein

R′ and R″ are divalent groups independently selected from the group consisting of null, optionally substituted C₂-C₈ alkynylene, optionally substituted C₃-C₁₀ carbocyclyl, optionally substituted 3-10 membered heterocyclyl, optionally substituted aryl, and optionally substituted heteroaryl. In some embodiments, R^{1′} is selected from the group consisting of C(O), optionally substituted C(O)—CH₂, optionally substituted pyridinyl, (optionally substituted pyridinyl)-(C₂ alkynylene), and (optionally substituted pyridinyl)-(optionally substituted piperazinyl)-. In another embodiment, R^{1′} is selected from the group consisting of C(O), C(O)—CH₂,

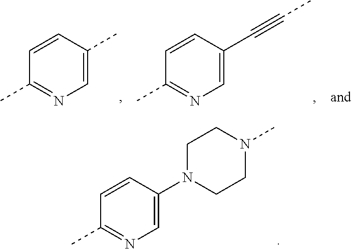

In some embodiments, R^{1′} is a bivalent group selected from optionally substituted C₁-C₈ alkylene, optionally substituted C₃-C₁₃ carbocyclyl, optionally substituted 3-13 membered heterocyclyl, optionally substituted aryl, and optionally substituted heteroaryl.

In some embodiments, R^{1′} is selected from C(O), optionally substituted C(O)—CH₂, and optionally substituted pyridinyl.

In some embodiments, R^{2′} is a bivalent group selected from null, CO, CON(R¹³), optionally substituted aryl, and optionally substituted heteroaryl. In some embodiments, R^{2′} is a bivalent group selected from optionally substituted aryl, and optionally substituted heteroaryl.

In some embodiments, R^{2′} is a bivalent group selected from the group consisting of null, CO, CONH, optionally substituted triazolyl, optionally substituted phenyl, optionally substituted pyridinyl, optionally substituted pyrazinyl, optionally substituted pyrimidinyl, optionally substituted pyridazinyl, triazinyl, optionally substituted pyrrolyl, furanyl, optionally substituted thiophenyl, optionally substituted imidazolyl, optionally substituted pyrazolyl, optionally substituted oxazolyl, optionally substituted isoxazolyl, optionally substituted thiazolyl, optionally substituted isothiazolyl, optionally substituted oxadiazolyl, optionally substituted thiadiazolyl, and optionally substituted tetrazolyl.

In some embodiments, R^{2′} is a bivalent group selected from the group consisting of optionally substituted triazolyl, optionally substituted phenyl, optionally substituted pyridinyl, optionally substituted pyrazinyl, optionally substituted pyrimidinyl, optionally substituted pyridazinyl, triazinyl, optionally substituted pyrrolyl, furanyl, optionally substituted thiophenyl, optionally substituted imidazolyl, optionally substituted pyrazolyl, optionally substituted oxazolyl, optionally substituted isoxazolyl, optionally substituted thiazolyl, optionally substituted isothiazolyl, optionally substituted oxadiazolyl, optionally substituted thiadiazolyl, and optionally substituted tetrazolyl.

In some embodiments, R^{2′} is a bivalent group selected from null, CO, CONH, optionally substituted triazolyl and optionally substituted phenyl. In some embodiments, R^{2′} is a bivalent group selected from optionally substituted triazolyl and optionally substituted phenyl.

In some embodiments, the substituent(s) for R^{2′} are independently optionally substituted groups selected from CN, F, Cl, Br, C₁-C₈ alkyl(such as C₁-C₄ alkyl), C₃-C₈ carbocyclyl (such as cyclopropyl), and C₁-C₈ haloalkyl (such as C₁-C₄ haloalkyl).

In some embodiments, R³ and R⁶ are independently selected from the group consisting of H, CN, halogen, optionally substituted C₁-C₆ alkyl, optionally substituted C₁-C₆ alkoxy, (optionally substituted C₁-C₆ alkyl)-S(O)₂—, (optionally substituted C₁-C₆ alkyl)-C(O)—, (optionally substituted C₁-C₆ alkyl)-NH—C(O)—, optionally substituted 3-6 membered carbocyclyl, and optionally substituted 3-6 membered heterocyclyl.

In some embodiments, the TYK2 ligand is a moiety of FORMULAE 1-1I, 1-1I, 1-2K, 2-1I, 2-1J or 2-2K; and R³ and R⁶ are independently selected from the group consisting of H, halogen, optionally substituted C₁-C₆ alkyl, optionally substituted 3-6 membered carbocyclyl, and optionally substituted 3-6 membered heterocyclyl.

In some embodiments, the TYK2 ligand is a moiety of FORMULAE 1-1I, 1-1J, 1-2K, 2-1I, 2-1J or 2-2K; and R³ and R⁶ are independently selected from the group consisting of H, F, Cl, Me, Et, iPr, and cPr.

In some embodiments, the TYK2 ligand is a moiety of FORMULAE 1-1I, 1-1J, 1-2K, 2-1I, 2-1J or 2-2K; and R²² is selected from optionally substituted NH₂, optionally substituted C₁-C₆ alkylamino, optionally substituted C₃-C₆ cycloalkylamino, optionally substituted C₁-C₆ alkyl, and optionally substituted 3-6 membered carbocyclyl.

In some embodiments, the TYK2 ligand is a moiety of FORMULAE 1-1I, 1-1J, 1-2K, 2-1I, 2-1J or 2-2K; and R²² is selected from NH₂, NHMe, NHCD₃, Me, Et, CD₃, CH₂CD₃, iPr, and cPr.

In some embodiments, the TYK2 ligand is a moiety of FORMULAE 1-1I, 1-1J, 1-2K, 2-1I, 2-1J or 2-2K: and R²² is selected from NH₂, NHMe, NHCD₃, Me, Et, iPr, and cPr.

In some embodiments, the TYK2 ligand is a moiety of FORMULAE 1-1I, 1-1J, 1-2K, 2-1I, 2-1J or 2-2K; and R²³ is selected from hydrogen, halogen, CN, NO₂, OR¹⁵, SR¹⁵, NR¹⁵R¹⁶, COR¹⁵, CON(R¹⁵)R¹⁶, SOR¹⁵, SO₂R¹⁵, SO₂N(R¹⁵)R¹⁶, optionally substituted C₁-C₈ alkyl, optionally substituted C₃-C₁₀ carbocyclyl, optionally substituted 3-10 membered heterocyclyl, wherein

R¹⁵ and R¹⁶ are independently selected from hydrogen, optionally substituted C₁-C₈ alkyl, optionally substituted C₃-C₁₀ carbocyclyl, optionally substituted 3-10 membered heterocyclyl, optionally substituted aryl, and optionally substituted heteroaryl.

In some embodiments, the TYK2 ligand is a moiety of FORMULAE 1-1I, 1-1J, 1-2K, 2-1I, 2-1J or 2-2K: and R²³ is selected from H, F, OMe, CONH₂, CONHMe, SMe, SOMe, SO₂Me, OCD₃, CONHCD₃, SCD₃, SOCD₃, and SO₂CD₃.

In some embodiments, the Degradation tag is a moiety of FORMULAE 6A, 6B, or 6C; and R_{EV}¹ is selected from isopropyl and tert-butyl.

In some embodiments, the Degradation tag is a moiety of FORMULA 6A-1, 6B-1, 6C-1, 6A-2, 6B-2, or 6C-2:

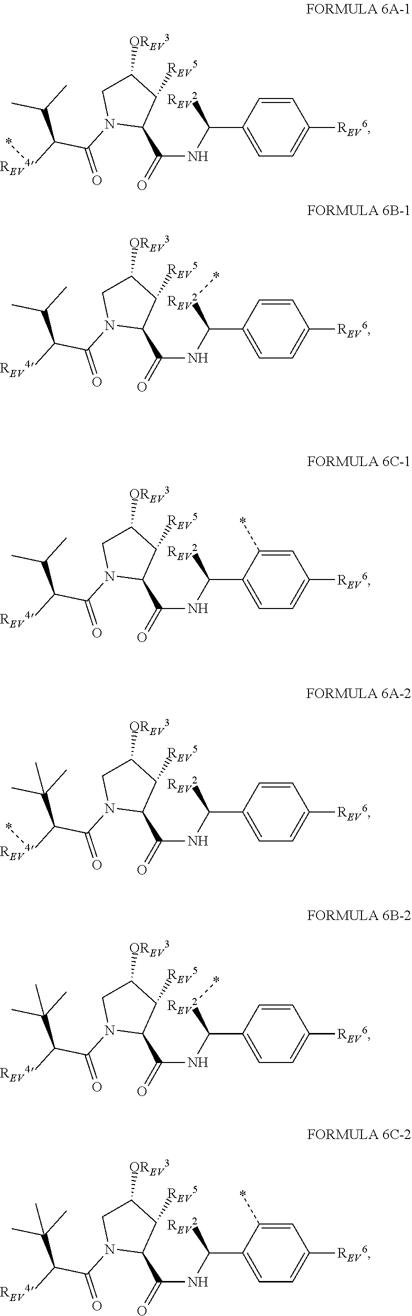

wherein R_{EV}², R_{EV}^{2′}, R_{EV}³, R_{EV}⁴, R_{EV}^{4′}, R_{EV}⁵, and R_{EV}⁶ are defined as in FORMULAE 6A, 6B, and 6C.

In some embodiments, R_{EV}² is optionally substituted C₁-C₈ alkyl; preferably, optionally substituted C₁-C₄ alkyl; more preferably, R_{EV}² is Me.

In some embodiments, R_{EV}² is H or Me. In some embodiments, R_{EV}² is Me.

In some embodiments, R_{EV}^{2′} is null or CH₂.

In some embodiments, the Degradation tag is a moiety of FORMULA 6A-3, 6B-3, 6C-3, 6A-4, 6B-4, or 6C-4:

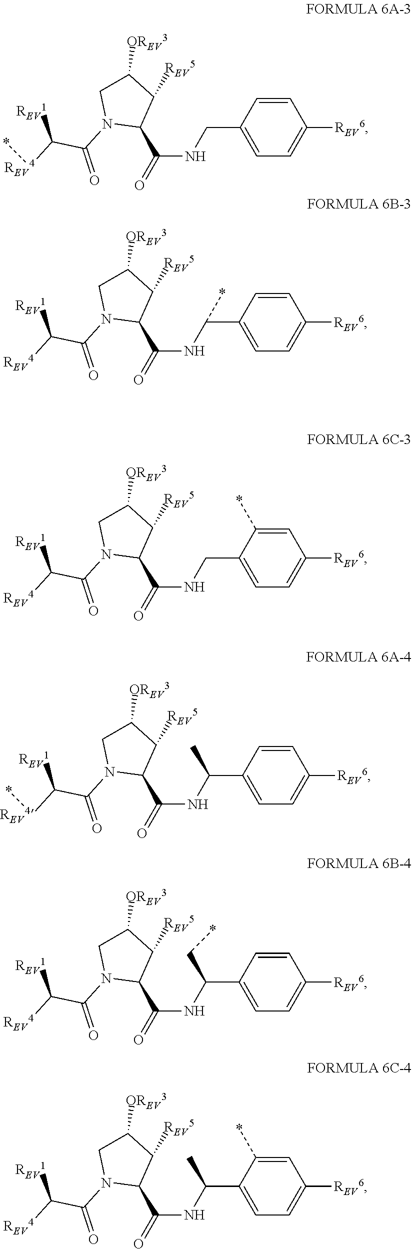

Wherein R_{EV}¹, R_{EV}³, R_{EV}⁴, R_{EV}^{4′}, R_{EV}⁵, and R_{EV}⁶ are defined as in FORMULAE 6A, 6B, and 6C.

In some embodiments, R_{EV}³ is H.

In some embodiments, the Degradation tag is a moiety of FORMULA 6A-5, 6B-5, or 6C-5:

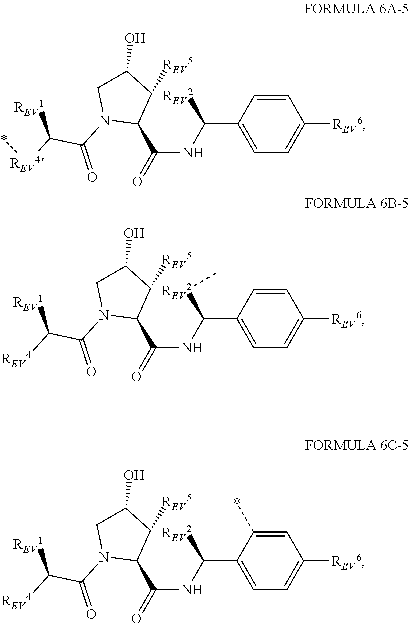

wherein

R_{EV}¹, R_{EV}², R_{EV}^{2′}, R_{EV}⁴, R_{EV}^{4′}, R_{EV}⁵, and R_{EV}⁶ are defined as in FORMULAE 6A, 6B, and 6C.

In some embodiments, R_{EV}⁵ is H or F; and preferably H.

In some embodiments, the Degradation tag is a moiety of FORMULA 6A-6, 6B-6, 6C-6, 6A-7, 6B-7, or 6C-7:

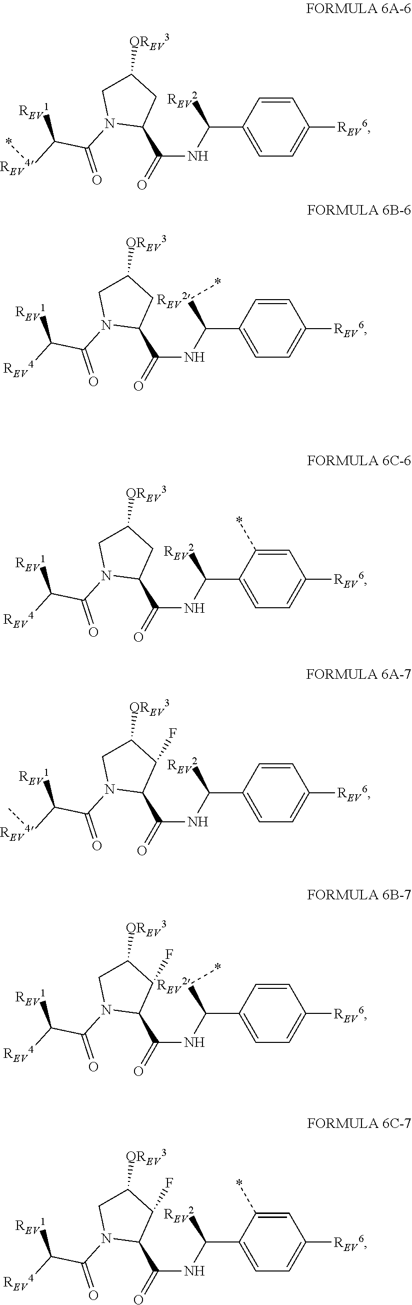

wherein

R_{EV}¹, R_{EV}², R_{EV}^{2′}, R_{EV}³, R_{EV}⁴, R_{EV}^{4′}, and R_{EV}⁶ are defined as in FORMULAE 6A, 6B, and 6C.

In some embodiments, R_{EV}⁶ is selected from hydrogen, halogen, cyano, optionally substituted aryl, and optionally substituted heteroaryl,

In some embodiments, R_{EV}⁶ is selected from the group consisting of halogen, cyano, optionally substituted thiazole, optionally substituted oxazole, optionally substituted imidazole, optionally substituted pyrazole, optionally substituted oxadiazole, optionally substituted triazole, and optionally substituted isoxazole.

In some embodiments, R_{EV}⁶ is methyl thiazole. In some embodiments, R_{EV}⁶ is

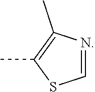

In some embodiments, the Degradation tag is a moiety of FORMULA 6A-8, 6B-8, or 6C-8:

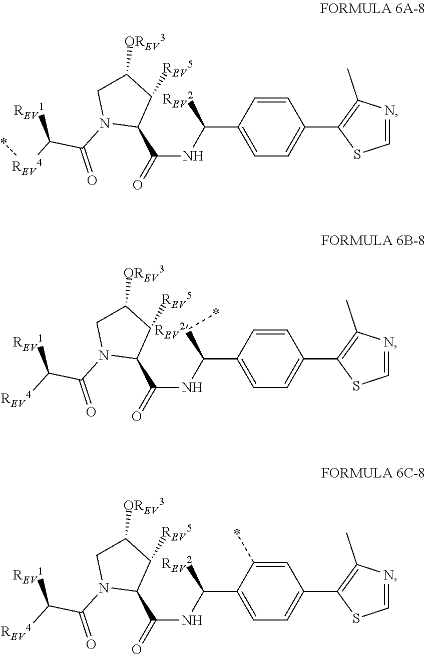

wherein

R_{EV}¹, R_{EV}², R_{EV}^{2′}, R_{EV}³, R_{EV}⁴, R_{EV}^{4′}, and R_{EV}⁵ are defined as in FORMULAE 6A, 6B, and 6C.

In some embodiments, R_{EV}⁴ is selected from —N(R_{EV}¹⁰)R_{EV}¹¹, —N(R_{EV}¹⁰)C(O)R_{EV}¹¹,

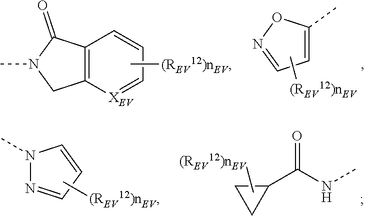

and/or R_{EV}^{4′} is selected from —N(R_{EV}¹⁰)—, —N(R_{EV}¹⁰)C(O)R_{EV}^{11′}—.

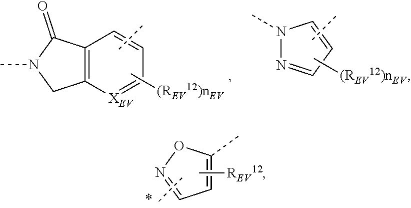

wherein

* indicates the connection to the linker moiety of the heterobifunctional compound;

R_{EV}¹⁰ is selected from null, hydrogen, optionally substituted C₁-C₈alkyl, optionally substituted C₁-C₈cycloalkyl, optionally substituted C₁-C₈alkyl-CO, optionally substituted C₃-C₈cycloalkyl-CO, optionally substituted C₃-C₈cycloalkyl-C₁-C₈alkyl-CO, optionally substituted 3-10 membered heterocyclyl-CO, optionally substituted 3-10 membered heterocyclyl-C₁-C₈alkyl-CO, optionally substituted aryl-CO, optionally substituted aryl-C₁-C₈alkyl-CO, optionally substituted heteroaryl-CO, optionally substituted heteroaryl-C₁-C₈alkyl-CO, optionally substituted aryl, and optionally substituted heteroaryl;

R_{EV}¹¹ is selected from null, hydrogen, optionally substituted C₁-C₈alkyl, and optionally substituted C₃-C₈cycloalkyl, and optionally substituted 3-8 membered heterocycloalkyl, optionally substituted C₃-C₈ carbocyclclyl, and optionally substituted C₃-C₈ heterocyclclyl;

R_{EV}^{11′}, at each occurrence, is a divalent group independently selected from null, O, optionally substituted C₁-C₈alkylene, optionally substituted C₃-C₈ cycloalkylene, optionally substituted C₃-C₈ heterocycloalkylene, optionally substituted C₃-C₈ carbocyclclyl, and optionally substituted C₃-C₈ heterocyclclyl;

R_{EV}¹², at each occurrence, is independently selected from hydrogen, halogen, cyano, optionally substituted C₁-C₈alkyl, optionally substituted C₃-C₈cycloalkyl, optionally substituted 3-8 membered heterocycloalkyl, optionally substituted C₁-C₈alkoxy, and optionally substituted C₃-C₈cycloalkoxy;

X_{EV} is selected from CH and N; and

n_{EV} is 0, 1, 2, 3, or 4.

In some embodiments, the substituent(s) for R_{EV}¹¹ and R_{EV}^{11′} are independently optionally substituted groups selected from C₁-C₄ alkyl, C₁-C₄haloalkyl, halogen, and CN.

In some embodiments, R_{EV}⁴ is selected from NH₂, NHC(O)Me,

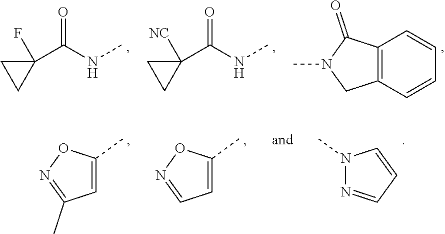

In some embodiments, R_{EV}^{4′} is selected from NH, C(O)NH, CH₂C(O)NH,

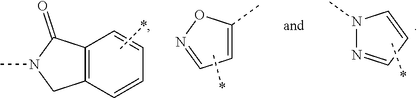

In some embodiments, the Degradation tag is a moiety of FORMULA 6A-9, 6A-10, 6A-11, 6A-12, 6A-13, 6B-9, 6B-10, 6B-11, 6B-12, 6B-13, 6B-14, 6B-15, 6C-9, 6C-10, 6C-11, 6C-12, 6C-13, 6C-14, or 6C-15:

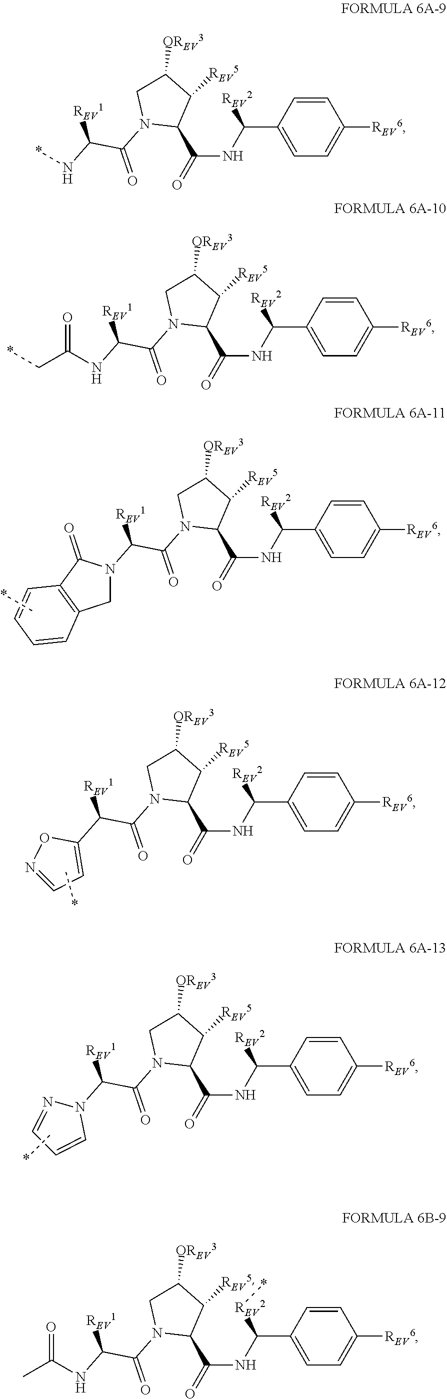

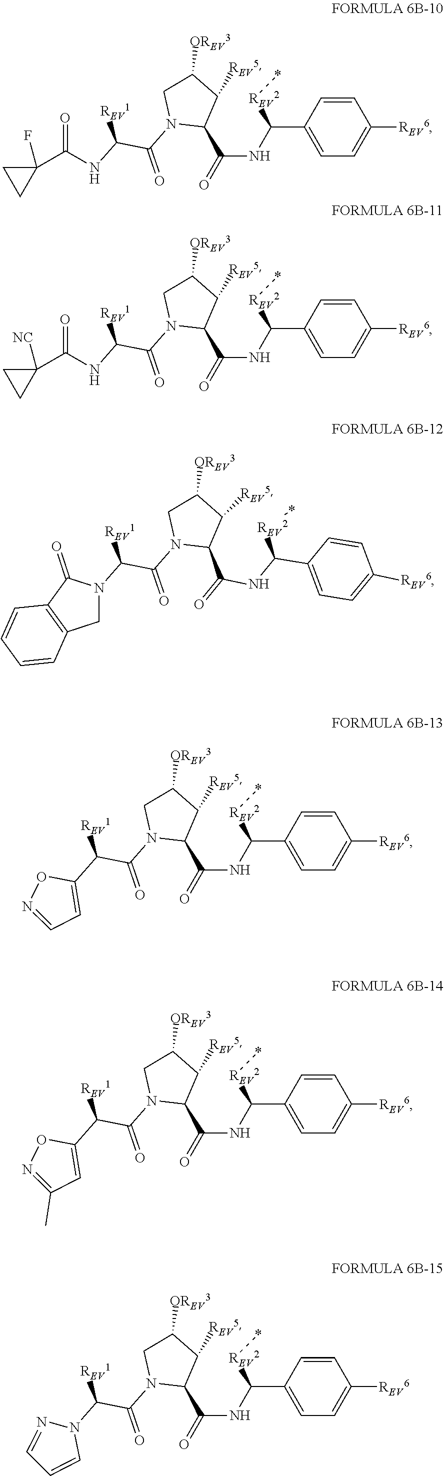

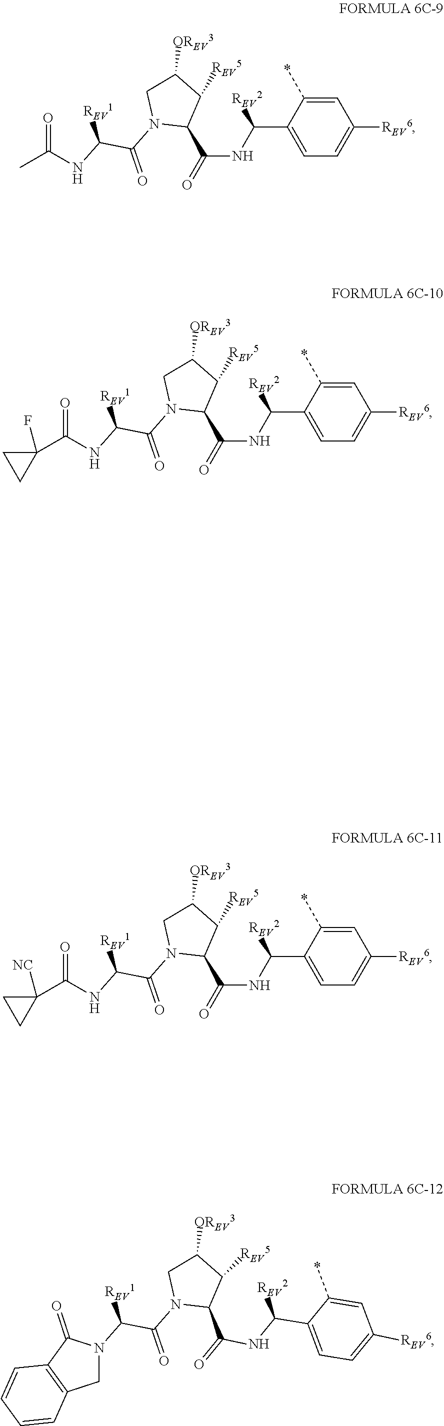

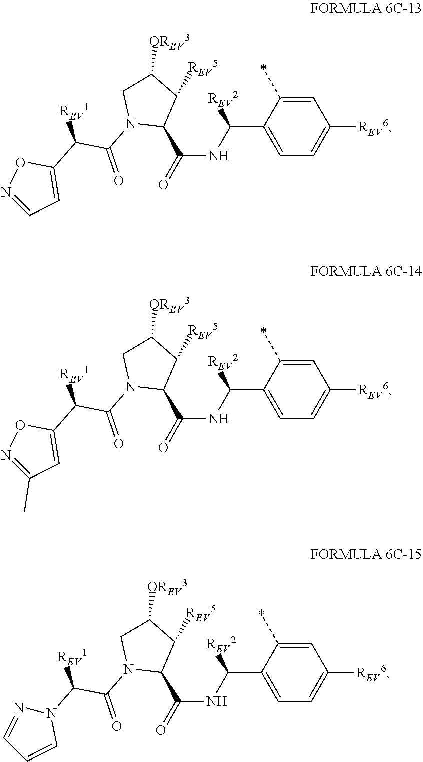

wherein R_{EV}¹, R_{EV}², R_{EV}^{2′}, R_{EV}³, R_{EV}⁵, and R_{EV}⁶ are defined as in FORMULAE 6A, 6B, and 6C.

In some embodiments, the Degradation tag is a moiety of any of FORMULAE 7A to 7BJ:

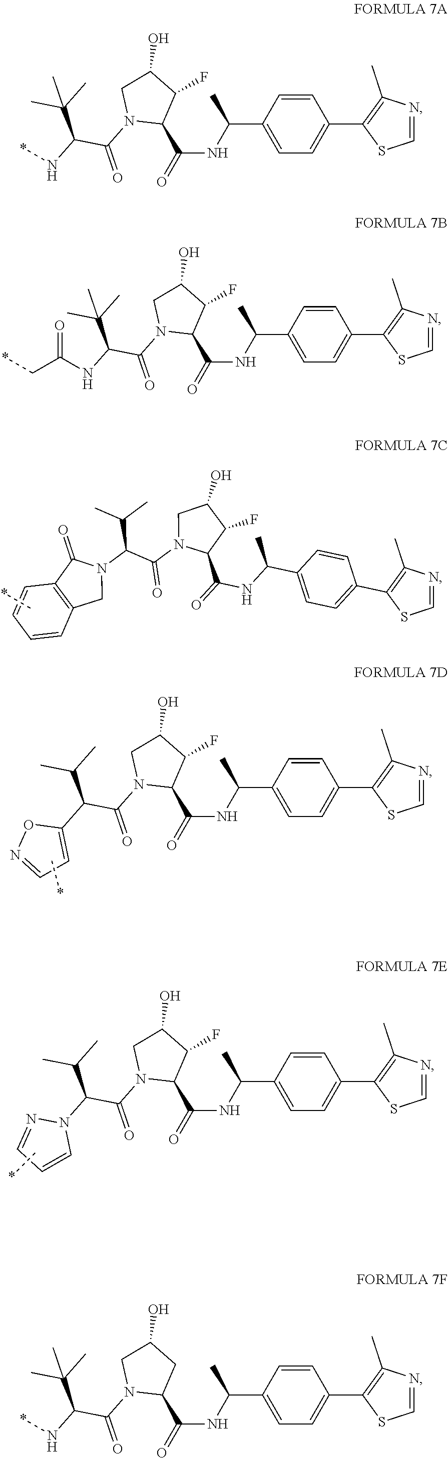

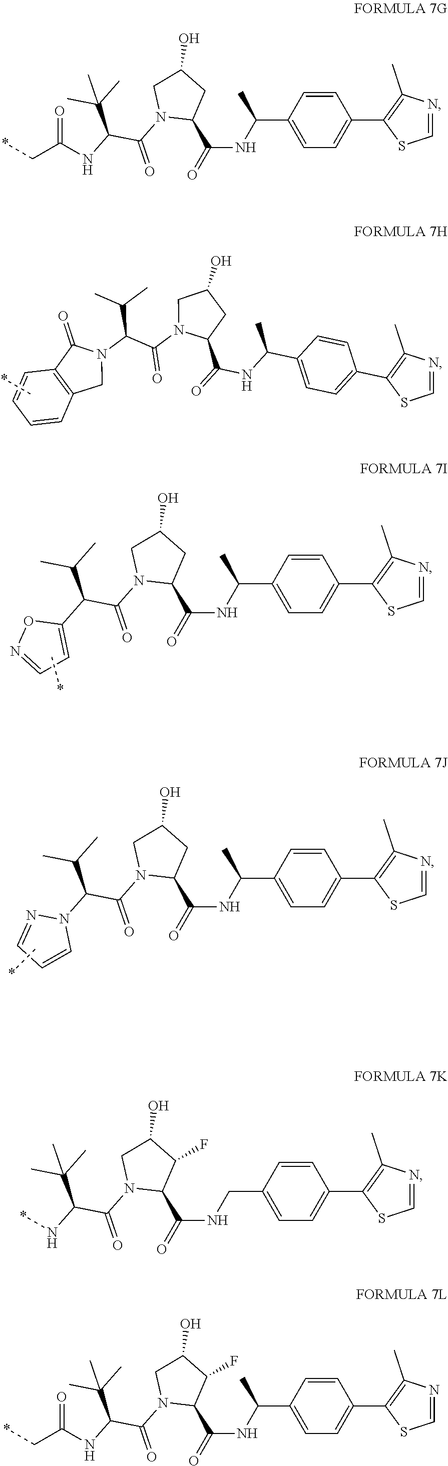

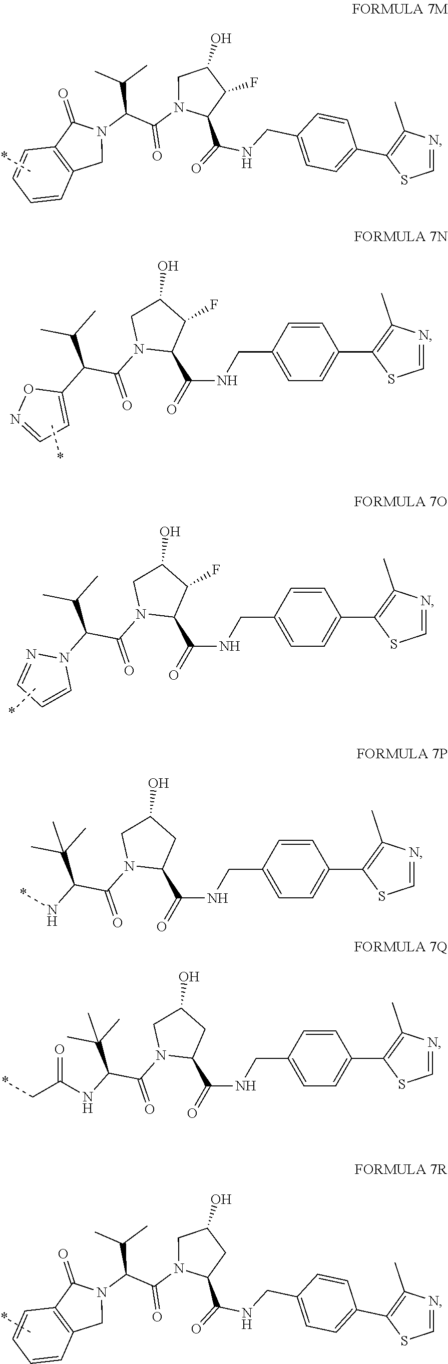

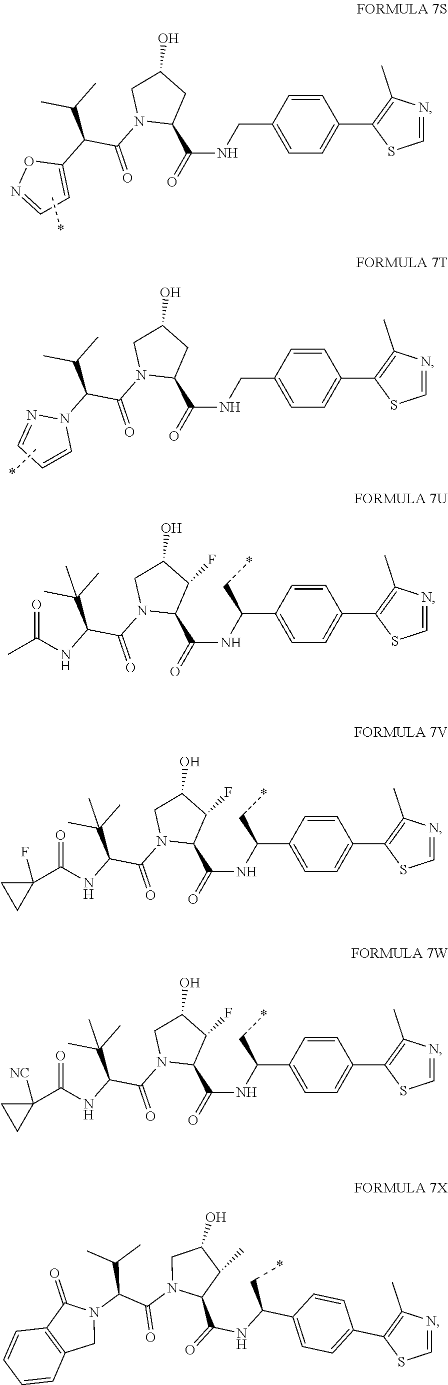

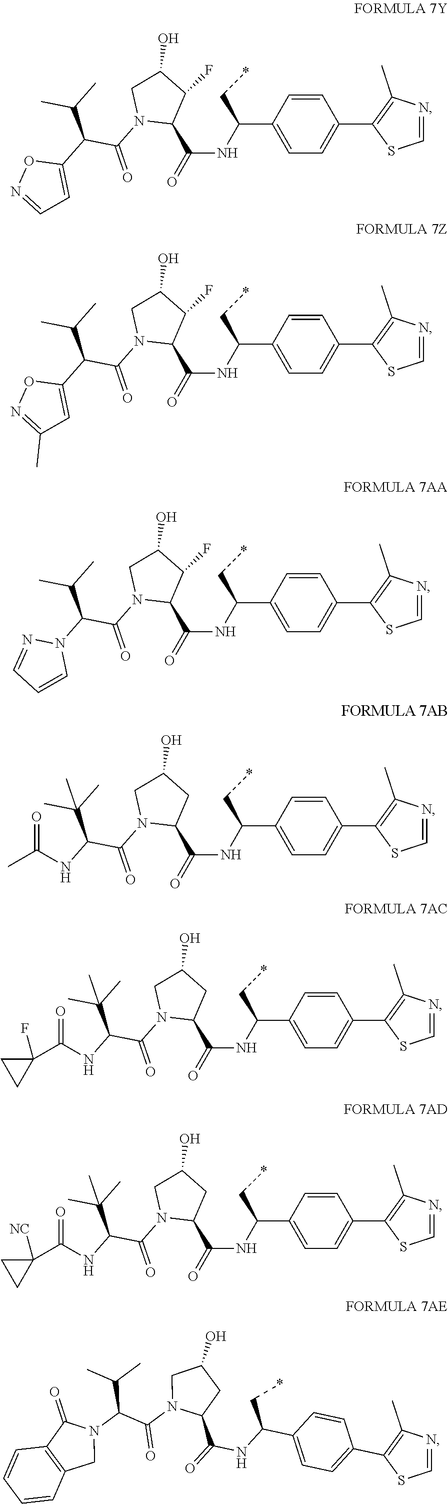

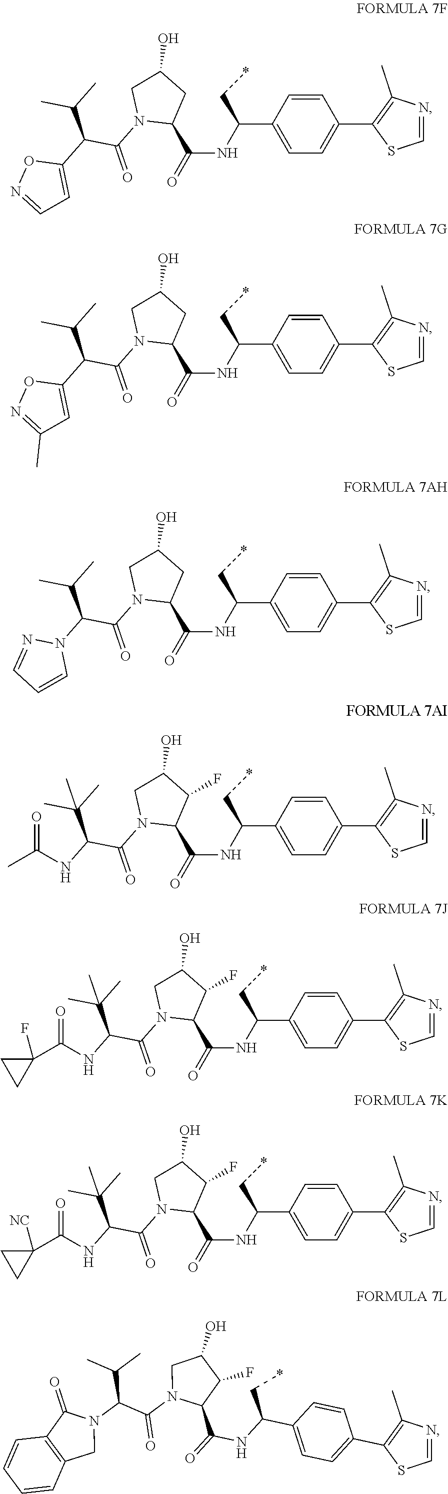

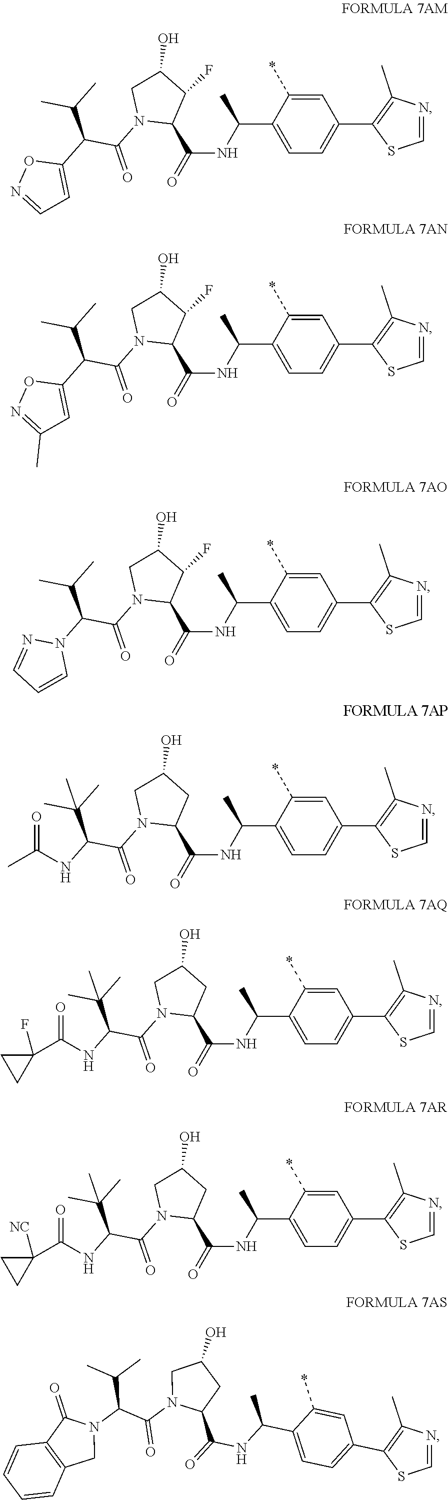

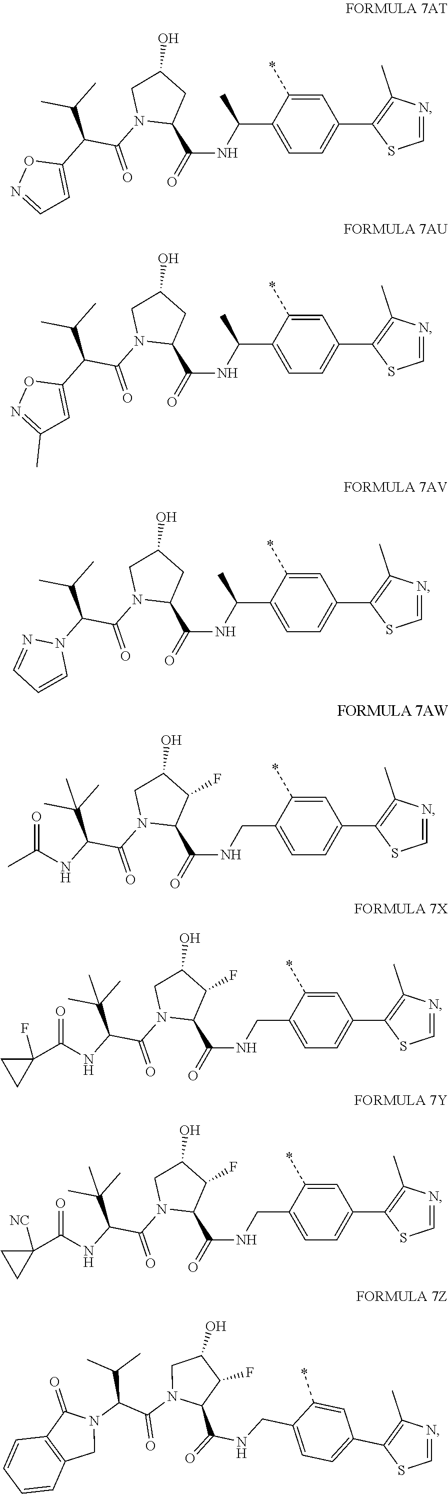

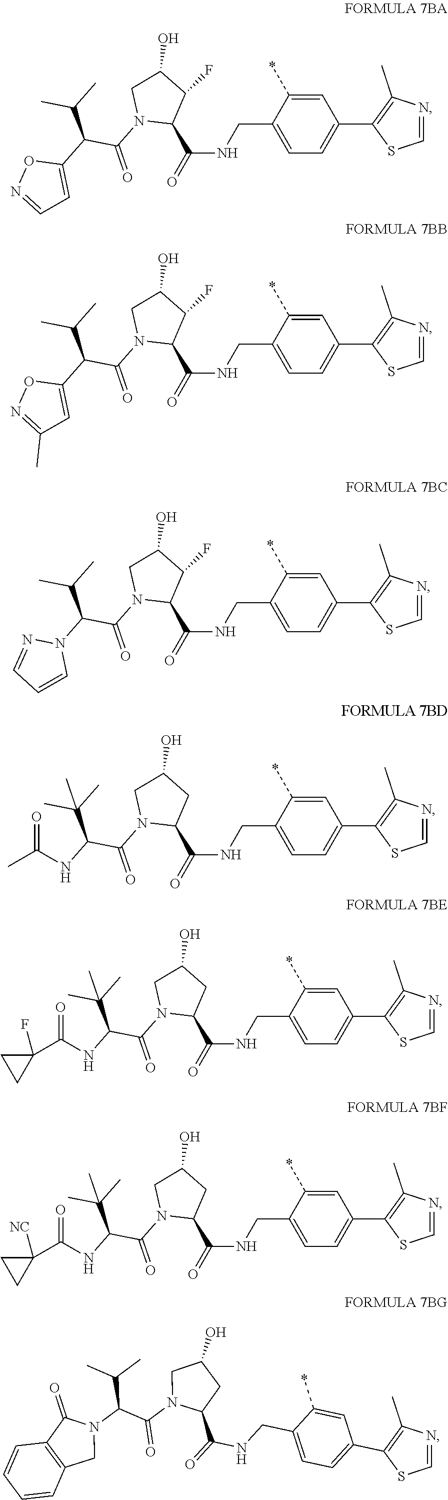

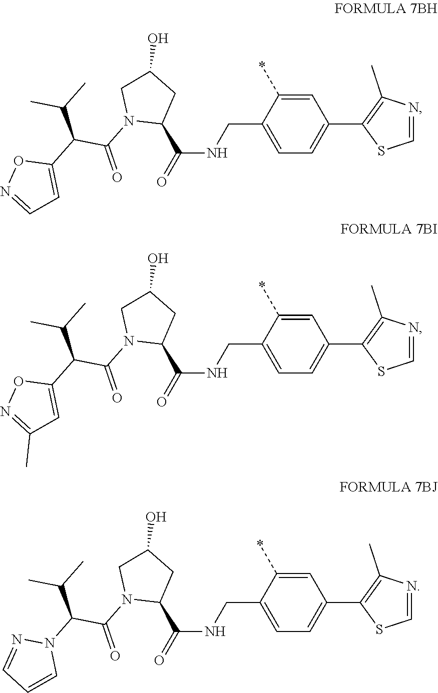

In another embodiment, the degradation tag is a moiety of FORMULA 5, and the degradation tag is connected to the linker moiety of the heterobifunctional compound via Z_{E};

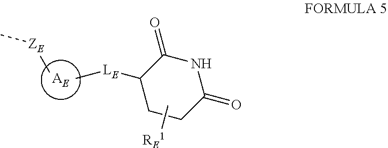

wherein

Z_{E} is a divalent group of —(R_{E}^{z})_{nE}—; wherein subscript n_{E}=0, 1, 2, 3, 4, 5 or 6; wherein R_{E}^{Z}, at each occurrence, is independently R_{E}^{r}, or R_{E}^{w}; wherein R_{E}^{w}, at each occurrence, is a bond or selected from the group consisting of —CO—, —CR_{E}⁵R_{E}⁶—, —NR_{E}⁵—, —O—, —S—, —S(O)—, —S(O)₂—, —C≡C—, optionally substituted C₁-C₁₀ alkylene, optionally substituted C₂-C₁₀ alkenylene, optionally substituted C₂-C₁₀ alkynylene; and R_{E}^{r}, at each occurrence, is a bond, or selected from the group consisting of optionally substituted C₃-C₁₀ carbocyclyl such as 3-13 membered carbocyclyl, optionally substituted 3-13 membered heterocyclyl, optionally substituted aryl, and optionally substituted heteroaryl (preferably, R_{E}^{w}, at each occurrence, is a bond or selected from the group consisting of —CO—, —CR_{E}⁵R_{E}⁶—, —NR_{E}⁵—, —O—, optionally substituted C₁-C₁₀ alkylene, optionally substituted C₂-C₁₀ alkenylene, optionally substituted C₂-C₁₀ alkynylene; and R_{E}^{r}, at each occurrence, is a bond, or selected from the group consisting of optionally substituted C₃-C₁₀ carbocyclyl such as 3-13 membered carbocyclyl, optionally substituted 3-13 membered heterocyclyl, optionally substituted aryl, and optionally substituted heteroaryl); with the proviso that —R_{E}^{z}—R_{E}^{z}— is not —O—O—;

R_{E}⁵ and R_{E}⁶, at each occurrence, are independently selected from the group consisting of hydrogen, halogen, oxo, hydroxy, amino, cyano, nitro, optionally substituted C₁-C₆ alkyl, optionally substituted C₁-C₈ alkoxy, optionally substituted C₁-C₈ alkylamino, optionally substituted 3 to 8 membered carbocyclyl, and optionally substituted 3 to 8 membered heterocyclyl; or R_{E}⁵ and R_{E}⁶, together with the atom(s) to which they are connected, optionally form an optionally substituted 3-8 membered cycloalkyl or optionally substituted 3-8 membered heterocyclyl (preferably, R_{E}⁵ and R_{E}⁶, at each occurrence, are independently selected from the group consisting of hydrogen, halogen, oxo, hydroxy, amino, cyano, nitro, optionally substituted C₁-C₆ alkyl, optionally substituted 3 to 8 membered carbocyclyl, and optionally substituted 3 to 8 membered heterocyclyl; or R_{E}⁵ and R_{E}⁶, together with the atom(s) to which they are connected, optionally form an optionally substituted 3-8 membered cycloalkyl or optionally substituted 3-8 membered heterocyclyl);

R_{E}¹ is selected from the group consisting of hydrogen, halogen, cyano, nitro, optionally substituted C₁-C₆ alkyl, optionally substituted C₁-C₈ heteroalkyl, optionally substituted 3-8 membered carbocyclyl, and optionally substituted 3-8 membered heterocyclyl (preferably, R_{E}¹ is selected from the group consisting of hydrogen, halogen, cyano, nitro, optionally substituted C₁-C₆ alkyl, optionally substituted 3-8 membered carbocyclyl, and optionally substituted 3-8 membered heterocyclyl);

L_{E} is a divalent group selected from the group consisting of null, -L_{E}¹-, and -L_{E}¹-L_{E}²-; wherein L_{E}¹ and L_{E}² are independently selected from the group consisting of —CO—, —O—, —CR_{E}¹⁰R_{E}¹¹— and —NR_{E}¹⁰—, with the proviso that -L_{E}¹-L_{E}²- is not —O—O—; wherein R_{E}¹⁰ and R_{E}¹¹ are independently selected from the group consisting of hydrogen, halogen, cyano, nitro, hydroxy, amino, optionally substituted C₁-C₆ alkyl, optionally substituted C₁-C₆ alkoxy, and optionally substituted C₁-C₆ alkylamino;

Ring A_{E} is a divalent group selected from the group consisting of FORMULA A_{E}1, A_{E}2, A_{E}3, A_{E}4, A_{E}5, A_{E}6 and A_{E}7 (preferably, Ring A_{E} is a divalent group selected from the group consisting of FORMULA A_{E}1, A_{E}2, A_{E}3, A_{E}4, and A_{E}5):

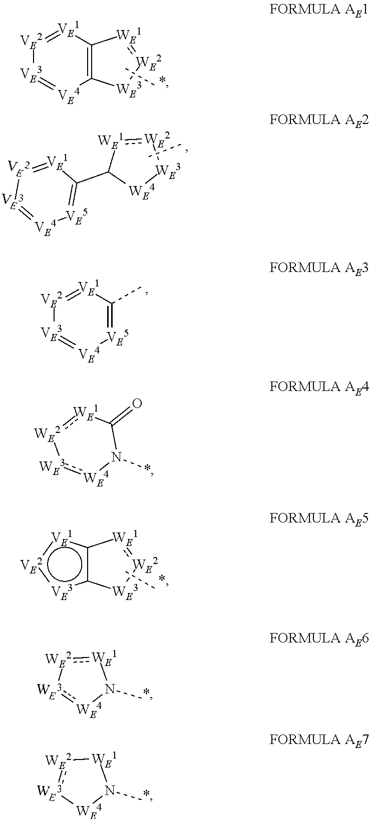

wherein

* indicates the attachment to L_{E}, and Z_{E} is attached to any possible position on the Ring A_{E},

[symbol] indicates a single bond or a double bond;

V_{E}¹, V_{E}², V_{E}³, V_{E}⁴ and V_{E}⁵, at each occurrence, are each independently selected from the group consisting of a bond, C, CR_{E}², S, N, and NR_{E}²; or V_{E}¹ and V_{E}², V_{E}² and V_{E}³, V_{E}³ and V_{E}⁴, or V_{E}⁴ and V_{E}⁵ are combined together to optionally form C₆ aryl ring or a 5, 6 or 7 membered heteroaryl ring;

R_{E}², at each occurrence, is independently selected from the group consisting of absent, hydrogen, halogen, cyano, nitro, hydroxy, amino, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, optionally substituted C₁-C₆ alkoxy, optionally substituted C₁-C₆ alkylamino, optionally substituted 3-8 membered carbocyclyl, and optionally substituted 3-8 membered heterocyclyl; or R_{E}² and another R_{E}² together with the atom(s) to which they are connected form optionally substituted 3-8 membered cycloalkyl, optionally substituted 3-8 membered heterocyclyl, optionally substituted aryl, and optionally substituted heteroaryl (preferably, R_{E}², at each occurrence, is independently selected from the group consisting of absent, hydrogen, halogen, cyano, nitro, hydroxy, amino, optionally substituted C₁-C₆ alkyl, optionally substituted C₂-C₆ alkenyl, optionally substituted C₂-C₆ alkynyl, optionally substituted C₁-C₆ alkoxy, optionally substituted C₁-C₆ alkylamino, optionally substituted 3-8 membered carbocyclyl, and optionally substituted 3-8 membered heterocyclyl; or R_{E}² and another R_{E}² together with the atom(s) to which they are connected form optionally substituted 3-8 membered cycloalkyl, optionally substituted 3-8 membered heterocyclyl, optionally substituted aryl, and optionally substituted heteroaryl);

W_{E}¹, W_{E}², W_{E}³ and W_{E}⁴ are each independently selected from the group consisting of —N═, —C—, —CR_{E}³═, —CO—, —O—, —CR_{E}³R_{E}⁴—, —NR_{E}³—, —CR_{E}³═CR_{E}⁴—, —N═CR_{E}³—, and —N═N—; or W_{E}¹ and W_{E}², W_{E}² and W_{E}³, or W_{E}³ and W_{E}⁴ are combined together to optionally form optionally substituted C₆ aryl or optionally substituted 5, 6 or 7 membered heteroaryl;

R_{E}³ and R_{E}⁴, at each occurrence, are independently selected from the group consisting of absent, hydrogen, halogen, cyano, nitro, hydroxy, amino, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, optionally substituted C₁-C₆ alkoxy, optionally substituted C₁-C₆ alkylamino, optionally substituted arylamino, optionally substituted heteroarylamino, optionally substituted 3 to 8 membered carbocyclyl, and optionally substituted 3 to 8 membered heterocyclyl (preferably, R_{E}³ and R_{E}⁴, at each occurrence, are independently selected from the group consisting of absent, hydrogen, halogen, cyano, nitro, hydroxy, amino, optionally substituted C₁-C₆ alkyl, optionally substituted 3 to 8 membered carbocyclyl, and optionally substituted 3 to 8 membered heterocyclyl); or R_{E}³ and R_{E}⁴, on the same atom or on the adjacent atoms, together with the atom(s) to which they are connected form an optionally substituted 3-8 membered cycloalkyl, optionally substituted 3-8 membered heterocyclyl ring, optionally substituted aryl, and optionally substituted heteroaryl (preferably, R_{E}³ and R_{E}⁴, on the same atom or on the adjacent atoms, together with the atom(s) to which they are connected form an optionally substituted 3-8 membered cycloalkyl or heterocyclyl ring).

In another embodiment, R_{E}² at each occurrence, is independently selected from the group consisting of absent, hydrogen, halogen, cyano, nitro, hydroxy, amino, optionally substituted C₁-C₆ alkyl, optionally substituted C₁-C₆ heteroalkyl, optionally substituted C₂-C₆ alkenyl, optionally substituted C₂-C₆ alkynyl, optionally substituted C₁-C₆ alkoxy, optionally substituted C₁-C₆ alkylamino, optionally substituted 3-8 membered carbocyclyl, and optionally substituted 3-8 membered heterocyclyl.

In another embodiment, R_{E}² at each occurrence, is independently selected from the group consisting of absent, hydrogen, halogen, cyano, nitro, hydroxy, amino, optionally substituted C₁-C₆ alkyl, optionally substituted C₂-C₆ alkenyl, optionally substituted C₂-C₆ alkynyl, optionally substituted C₁-C₆ alkoxy, optionally substituted C₁-C₆ alkylamino, optionally substituted 3-8 membered carbocyclyl, and optionally substituted 3-8 membered heterocyclyl.

In another embodiment, the degradation tag is a moiety of FORMULA 5, and wherein V_{E}¹, V_{E}², V_{E}³, V_{E}⁴ and V_{E}⁵, at each occurrence, are each independently selected from the group consisting of C, CR_{E}², S, N, and NR_{E}²; or V_{E}¹ and V_{E}², V_{E}² and V_{E}³, V_{E}³ and V_{E}⁴, or V_{E}⁴ and V_{E}⁵ are combined to optionally form C₆ aryl ring or a 5, 6 or 7 membered heteroaryl ring.

In another embodiment, the degradation tag is a moiety of FORMULA 5, and wherein Ring A_{E} is a group consisting of FORMULA A_{E}1, and wherein V_{E}¹, V_{E}², V_{E}³, and V_{E}⁴ are each independently selected from the group consisting of C, CR_{E}², S, N, and NR_{E}².

In another embodiment, the degradation tag is a moiety of FORMULA 5, and wherein Ring A_{E} is a group consisting of FORMULA A_{E}2, and wherein V_{E}¹, V_{E}², V_{E}³, V_{E}⁴ and V_{E}⁵, at each occurrence, are each independently selected from the group consisting of C, CR_{E}², S, N, and NR_{E}².

In another embodiment, the degradation tag is a moiety of FORMULA 5, and wherein Ring A_{E} is a group consisting of FORMULA A_{E}3, and wherein V_{E}¹, V_{E}², V_{E}³, Y_{E}⁴ and V_{E}⁵ are each independently selected from the group consisting of C, CR_{E}², S, N, and NR_{E}²; or V_{E}¹ and V_{E}², V_{E}² and V_{E}³, V_{E}³ and V_{E}⁴, or V_{E}⁴ and V_{E}⁵ are combined together to optionally form C₆ aryl ring or a 5, 6 or 7 membered heteroaryl ring.

In another embodiment, the degradation tag is a moiety of FORMULA 5, and wherein Ring A_{E} is a group consisting of FORMULA A_{E}4, and wherein [symbol] is a single bond and W_{E}¹, W_{E}², W_{E}³ and W_{E}⁴ are each independently selected from the group consisting of —N═, —CR_{E}³═, —CO—, —O—, —CR_{E}³R_{E}⁴—, and —NR_{E}³—.

In another embodiment, the degradation tag is a moiety of FORMULA 5, and wherein Ring A_{E} is a group consisting of FORMULA A_{E}5, and wherein V_{E}¹, V_{E}², and V_{E}³ are each independently selected from the group consisting of CR_{E}², S, N, and NR_{E}², with the proviso that at least one of V_{E}¹, V_{E}², and V_{E}³ is S, N or NR_{E}²; or V_{E}¹ and V_{E}², V_{E}² and V_{E}³ are combined together to optionally form 5 membered heteroaryl ring.

In another embodiments, the degradation tag is a moiety of FORMULA 5, and wherein Ring A_{E} is a group consisting of Formula A_{E}1, A_{E}2, and A_{E}5, and W_{E}¹, W_{E}², W_{E}³ and W_{E}⁴ are each independently selected from the group consisting of —N═, —CR_{E}³═, —CO—, —O—, —S—, —CR_{E}³R_{E}⁴—, and —NR_{E}³—.

In another embodiments, the degradation tag is a moiety of FORMULA 5, and wherein Ring A_{E} is a group consisting of Formula A_{E}6, and wherein [symbol] is a double bond and W_{E}¹, W_{E}², W_{E}³ and W_{E}⁴ are each independently selected from the group consisting of —N═, —CR_{E}³═.

In another embodiments, the degradation tag is a moiety of FORMULA 5, and wherein Ring A_{E} is a group consisting of Formula A_{E}⁷, and wherein [symbol] is a double bond and W_{E}¹ and W_{E}⁴ are independently selected from —CO—, and CR_{E}³R_{E}⁴—; and W_{E}² and W_{E}³ are independently selected from the group consisting of —N═, and —CR_{E}³═.

In another embodiments, the degradation tag is a moiety of FORMULA 5, and wherein Ring A_{E} is a group consisting of Formula A_{E}7, and wherein [symbol] is a single bond and W_{E}¹, W_{E}², W_{E}³ and W_{E}⁴ are each independently selected from the group consisting of —CO—, —O—, —CR_{E}³R_{E}⁴—, and —NR_{E}³—.

In another embodiment, the degradation tag is a moiety of FORMULA 5, and wherein R_{E}¹ is selected from hydrogen, halogen, cyano, nitro, optionally substituted C₁-C₆ alkyl, optionally substituted 3-8 membered carbocyclyl, and optionally substituted 3-8 membered heterocyclyl; preferably, R_{E}¹ is selected from hydrogen, halogen, cyano, nitro, and C₁-C₅ alkyl; more preferably, R_{E}¹ is selected from H, CH₃, or F.

In another embodiment, the degradation tag is a moiety of FORMULA 5, and wherein R_{E}² is selected from hydrogen, halogen, cyano, nitro, hydroxy, amino, optionally substituted C₁-C₆ alkyl, optionally substituted C₁-C₆ heteroalkyl, optionally substituted C₁-C₆ alkoxyl, optionally substituted C₁-C₆ alkylamino, optionally substituted 3 to 8 membered carbocyclyl, and optionally substituted 3 to 8 membered heterocyclyl; preferably, R_{E}² is selected from hydrogen, halogen, cyano, nitro, and C₁-C₆ alkyl, optionally substituted C₁-C₆ alkoxyl, optionally substituted 3 to 8 membered carbocyclyl, and optionally substituted 3 to 8 membered heterocyclyl; more preferably, R_{E}² is selected from H, F, Cl, Me, OMe, OCF₃, O-iPr, or O-cPr (further preferably, R_{E}² is selected from H, F, Cl, Me, OMe, OCF₃, O-iPr, or O-cPr).

In another embodiments, the degradation tag is a moiety of FORMULA 5, and wherein two adjacent R_{E}² together with the atom(s) to which they are connected optionally form optionally substituted 3 to 8 membered cycloalkyl, optionally substituted 3 to 8 membered heterocyclyl, optionally substituted aryl, and optionally substituted heteroaryl.

In another embodiment, the degradation tag is a moiety of FORMULA 5, and wherein R_{E}³ and R_{E}⁴, at each occurrence, are independently selected from hydrogen, halogen, cyano, nitro, optionally substituted C₁-C₆ alkyl, optionally substituted C₁-C₆heteroalkyl, optionally substituted arylamino, optionally substituted 3 to 8 membered carbocyclyl, and optionally substituted 3 to 8 membered heterocyclyl; or two independent R_{E}³, two independent R_{E}⁴, or R_{E}³ and R_{E}⁴ together with the atom(s) to which they are connected form a 3-8 membered carbocyclyl, or 3-8 membered heterocyclyl.

In another embodiment, the degradation tag is a moiety of FORMULA 5, and wherein R_{E}³ and R_{E}⁴, at each occurrence, are independently selected from hydrogen, halogen, cyano, nitro, optionally substituted C₁-C₆ alkyl, optionally substituted 3 to 8 membered carbocyclyl, and optionally substituted 3 to 8 membered heterocyclyl; or R_{E}³ and R_{E}⁴ together with the atom(s) to which they are connected form a 3-8 membered carbocyclyl, or 3-8 membered heterocyclyl.

In some embodiments, R_{E}³ and R_{E}⁴, at each occurrence, are independently selected from H, F, or Me.

In another embodiment, R_{E}^{r}, at each occurrence, is selected from Group R_{E}, and

Group R_{E} consists of optionally substituted following cyclic groups

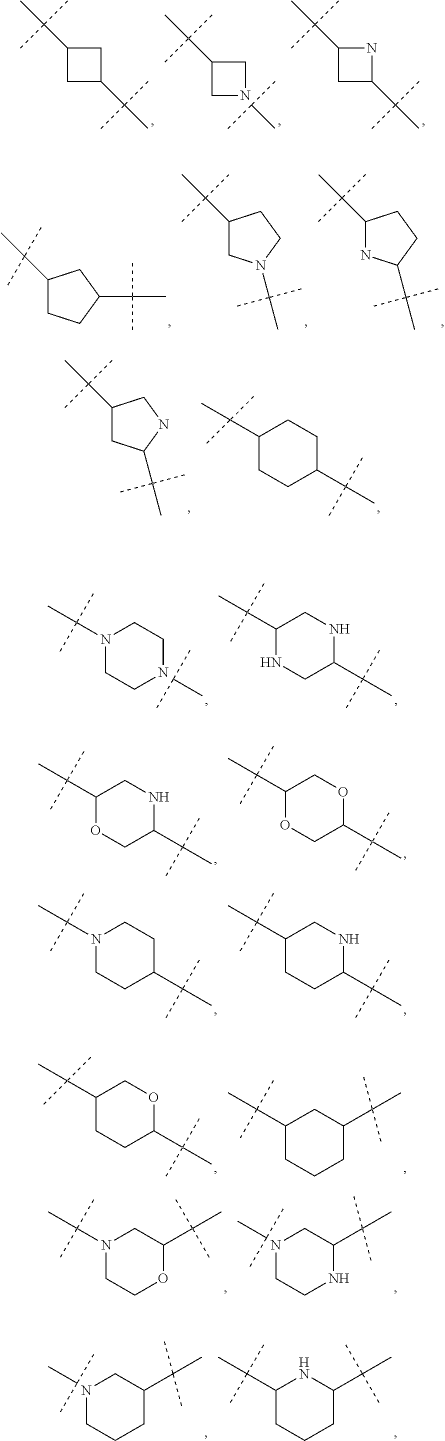

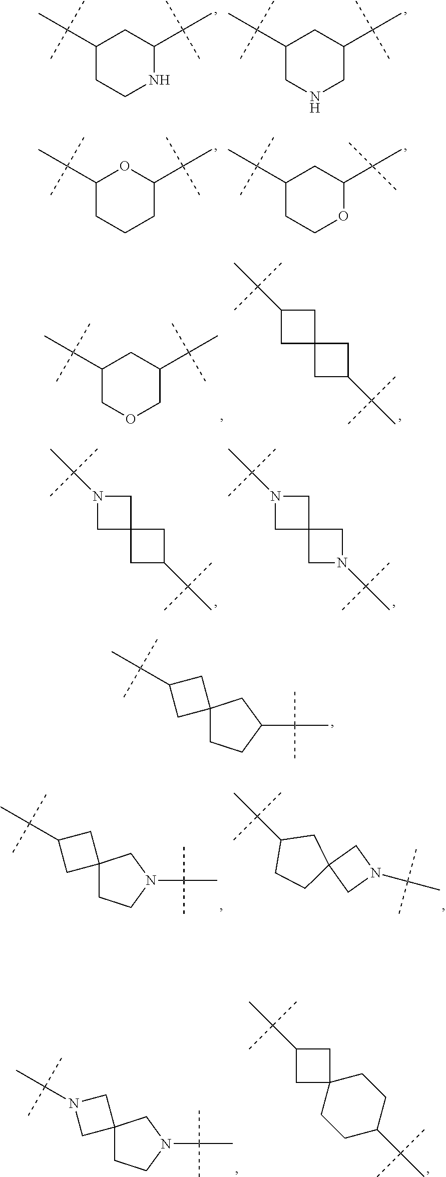

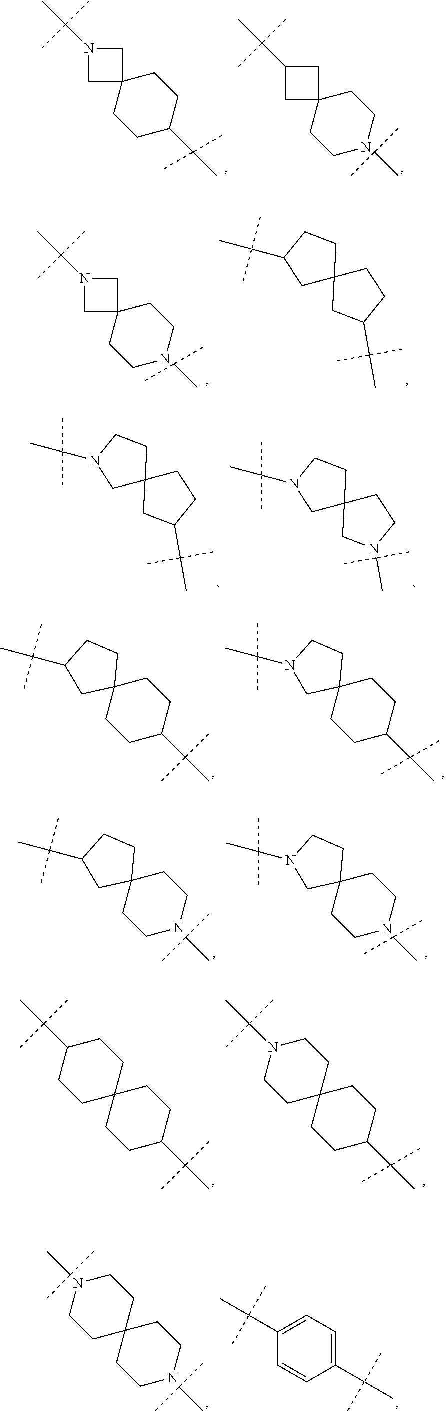

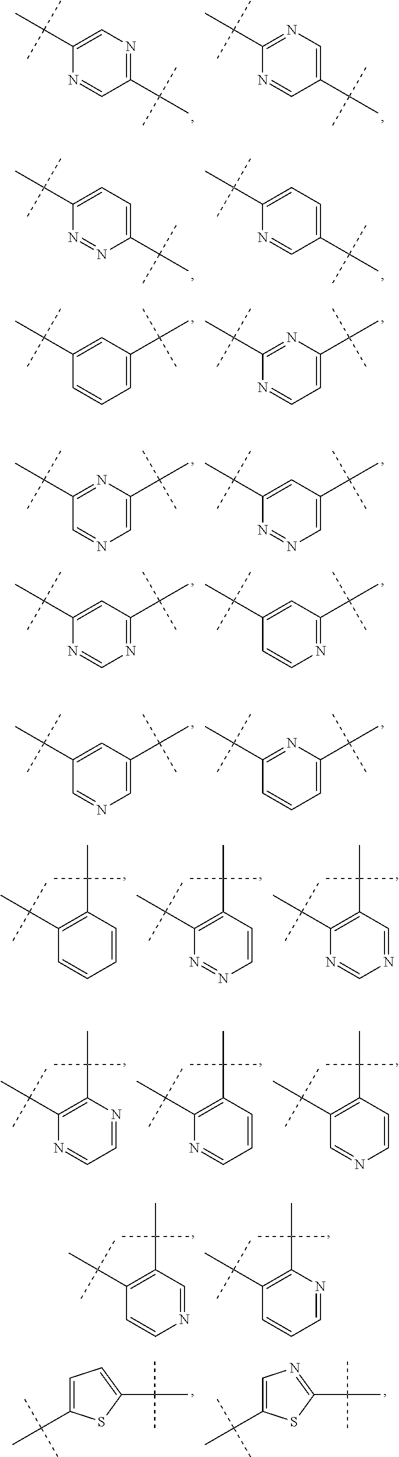

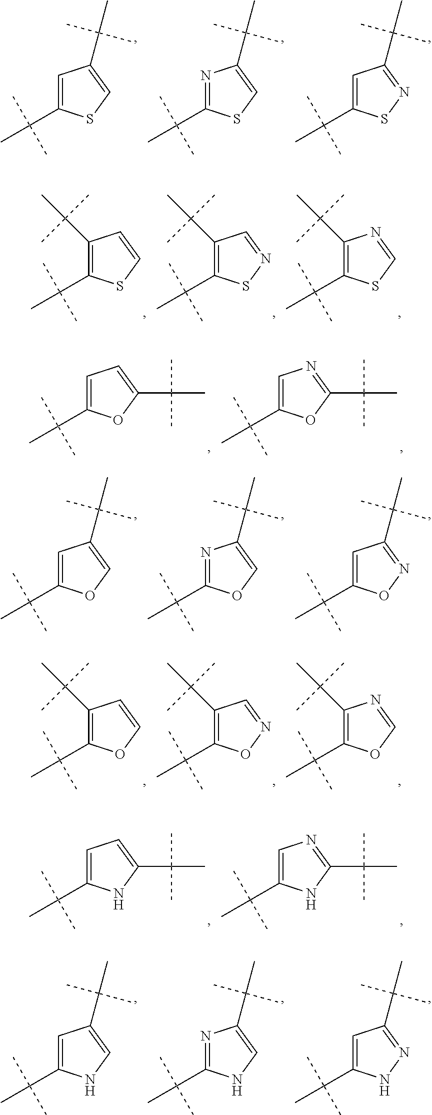

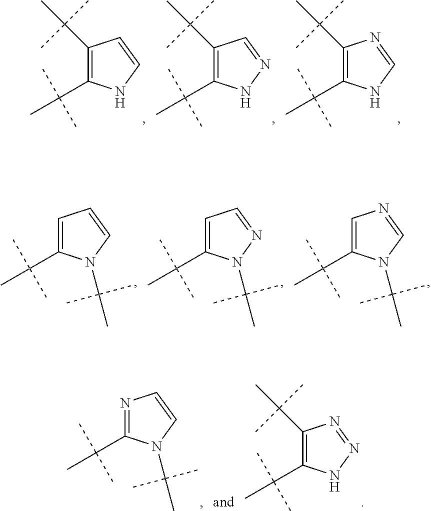

In another embodiment, the degradation tag is a moiety of FORMULA 5, and wherein in the group of Z_{E}, at most one R_{E}^{Z} is R_{E}^{r}.

In another embodiment, the degradation tag is a moiety of FORMULA 5, and wherein n_{E}=0, 1, 2 or 3.

In another embodiment, the degradation tag is a moiety of FORMULA 5, and wherein Z_{E} is a divalent group selected from the group consisting of —Rₑ^{w}—, —(R_{E}^{w})₂—, —(R_{E}^{w})₃—, —R_{E}^{r}—, —R_{E}^{w}—R_{E}^{r}R_{E}^{w}—, —R_{E}^{r}R_{E}^{w}— and —R_{E}^{r}—(R_{E}^{w})₂—.

In another embodiment, the degradation tag is a moiety of FORMULA 5, and wherein R_{E}⁵ and R_{E}⁶ at each occurrence are independently selected from a bond, hydrogen, halogen, oxo, hydroxyl, amino, cyano, nitro, optionally substituted C₁-C₆ alkyl, optionally substituted 3 to 8 membered carbocyclyl, and optionally substituted 3 to 8 membered heterocyclyl; or R_{E}⁵ and R_{E}⁶ together with the atom(s) to which they are connected form a 3-8 membered cycloalkyl or heterocyclyl ring.

In another embodiment, the degradation tag is a moiety of FORMULA 5, and wherein R_{E}^{Z} is selected from —CO—, —CR_{E}⁵R_{E}⁶—, —NR_{E}⁵—, —O—, optionally substituted C₁-C₁₀ alkylene, optionally substituted C₁-C₁₀ alkenylene, optionally substituted C₁-C₁₀ alkynylene, optionally substituted 3-8 membered carbocyclyl, optionally substituted 3-8 membered heterocyclyl.

In another embodiment, the degradation tag is a moiety of FORMULA 5, and wherein Z_{E} is selected from a bond, CH, CH═CH, C≡C, NH, and O.

In another embodiment, the degradation tag is a moiety of FORMULA 5, and wherein Ring A_{E} is of FORMULA A_{E}1, A_{E}2, A_{E}3, A_{E}4, A_{E}5, A_{E}6; and L_{E} is null.

In another embodiment, the degradation tag is a moiety of FORMULA 5, and wherein Ring A_{E} is of FORMULA A_{E}3 and L_{E} is not null.

In another embodiment, the degradation tag is a moiety of FORMULA 5, and wherein Ring A_{E} is of FORMULA A_{E}3 and L_{E} is selected from the group consisting of —NH—, —N(C₁-C₄ alkyl)-, —CO—, —NH—CO—, —N(C₁-C₄ alkyl)-CO—, —CO—NH—, and —CO—N(C₁-C₄ alkyl)-.

In another embodiment, the degradation tag is a moiety selected from the groups consisting of FORMULAE 5-1, 5-2, 5-3, 5-4, 5-5, 5-6, 5-7, 5-8, and 5-9; and the degradation tag is connected to the linker moiety of the heterobifunctional compound via a divalent group of Z_{E};

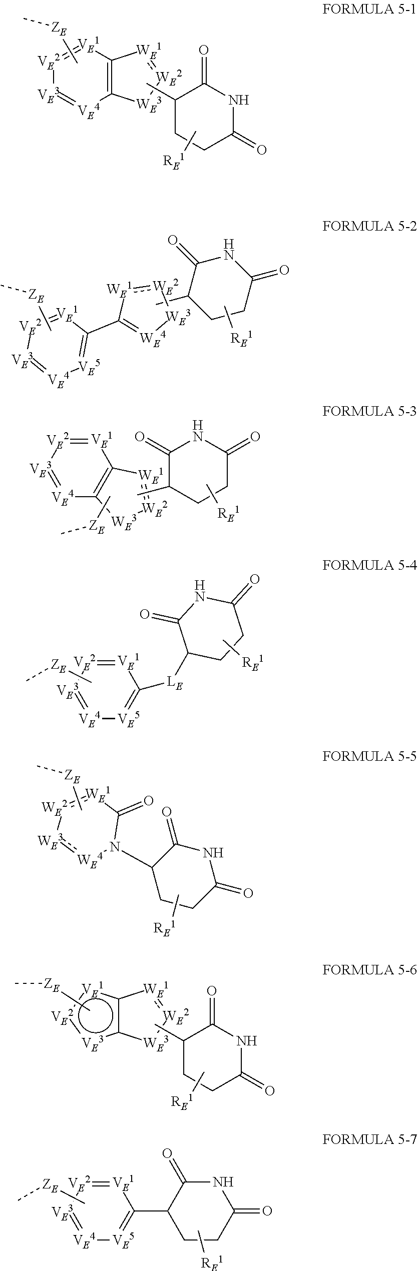

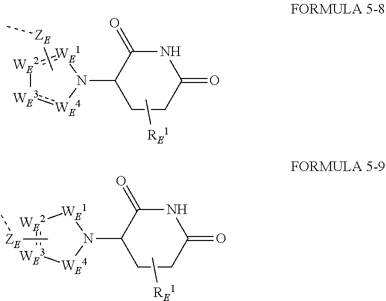

wherein

Z_{E}, R_{E}¹. L_{E}, [symbol] , V_{E}¹, V_{E}², V_{E}³, V_{E}⁴, V_{E}⁵, W_{E}¹, W_{E}², W_{E}³ and W_{E}⁴ are defined as in FORMULA 5.

In another embodiment, the degradation tag is a moiety selected from the group consisting of FORMULAE 5A, 5B, 5C, 5D, 5E, 5F, 5G, 5H, 5I, 5J, 5K, 5L, 5M, 5N, 5O, and 5P:

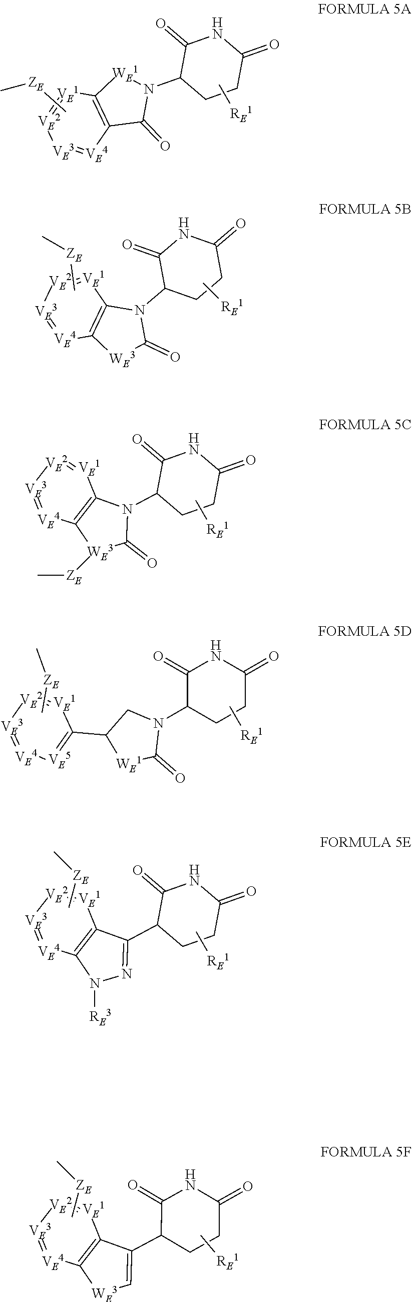

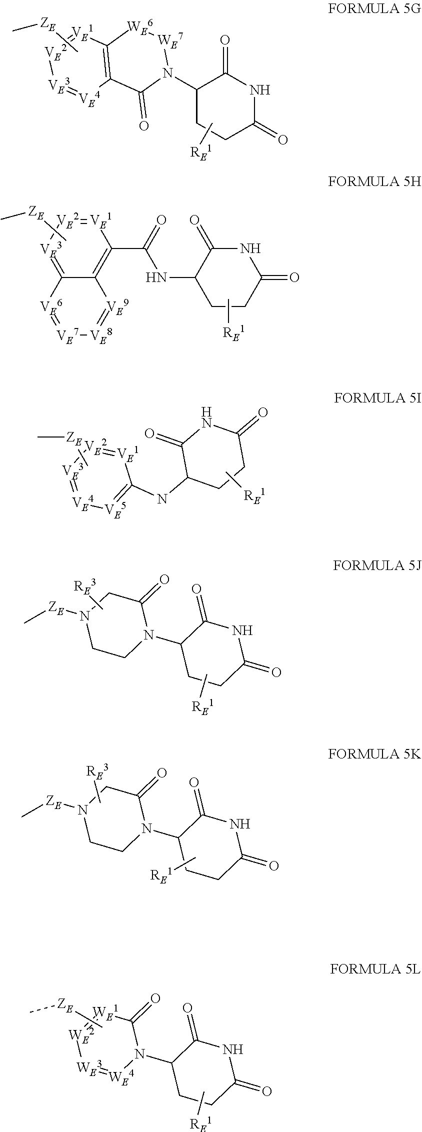

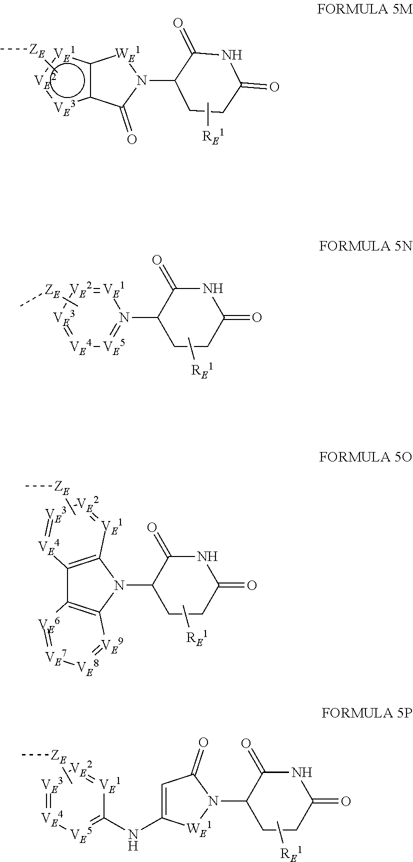

wherein,

V_{E}⁶, V_{E}⁷, V_{E}⁸, and V_{E}⁹ are each independently selected from a bond, C, CR_{E}¹² and N; or V_{E}¹ and V_{E}², V_{E}² and V_{E}³, V_{E}³ and V_{E}⁴, or V_{E}⁴ and V_{E}⁵ are combined together to optionally form C₆ aryl ring or a 5, 6 or 7 membered heteroaryl ring;

R_{E}¹², at each occurrence, is independently selected from the group consisting of hydrogen, halogen, cyano, nitro, hydroxy, amino, optionally substituted C₁-C₆ alkyl, optionally substituted C₁-C₆ alkenyl, optionally substituted C₁-C₆ alkynyl, optionally substituted C₁-C₆ alkoxy, optionally substituted C₁-C₆ alkylamino, optionally substituted 3-8 membered carbocyclyl, and optionally substituted 3-8 membered heterocyclyl;

W_{E}⁶ and W_{E}⁷ are each independently selected from —CR_{E}²═ and —N═;

W_{E}¹, W_{E}², W_{E}³, W_{E}⁴, V_{E}¹, V_{E}², V_{E}³, V_{E}⁴, V_{E}⁵, R_{E}¹, R_{E}³, and Z_{E} are defined as in FORMULA 5.

In another embodiment, W_{E}¹ is selected from —CO—, —O—, —CR_{E}³R_{E}⁴—, —NR_{E}³—, —CR_{E}³═CR_{E}⁴—, —N═CR_{E}³—, and —N≡N—.

In another embodiment, Ring A_{E} is a divalent group of FORMULA A_{E}1 or A_{E}5; and Ring A_{E} is attached to L_{E} via W_{E}².

In another embodiment, Ring A_{E} is a divalent group of FORMULA A_{E}1 or A_{E}5, wherein W_{E}¹ and W_{E}³ are each independently selected from the group consisting of CO, O, CR_{E}³R_{E}⁴, NR_{E}³; and W_{E}² is N.

In another embodiment, the degradation tag is a moiety of FORMULA 5-1 or 5-6, and the degradation tag is connected to the linker moiety of the heterobifunctional compound via a divalent group of Z_{E}; wherein

W_{E}¹ and W_{E}³ are each independently selected from the group consisting of —CO—, —O—, —CR_{E}³R_{E}⁴—, —NR_{E}³—;

W_{E}² is N, and connected to

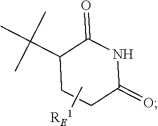

Z_{E}, R_{E}¹. R_{E}³. R_{E}⁴L_{E}, [symbol] , V_{E}¹, V_{E}², V_{E}³, V_{E}⁴, and V_{E}⁵, are defined as in FORMULA 5.

In another embodiment, the degradation tag is a moiety of FORMULAE 5A or 5M; wherein W_{E}¹ is independently selected from the group consisting of —CO—, —O—, —CR_{E}³R_{E}⁴—, —NR_{E}³—; and V_{E}¹, V_{E}², V_{E}³, V_{E}⁴, R_{E}¹, R_{E}³, R_{E}⁴ and Z_{E} are defined as in FORMULA 5.

In another embodiment, R_{E}³ and R_{E}⁴, at each occurrence, are independently selected from the group consisting of absent, hydrogen, halogen, cyano, nitro, hydroxy, amino, optionally substituted C₁-C₆ alkyl.

In another embodiment, the degradation tag is a moiety of FORMULA 5-1, or FORMULA 5-3,

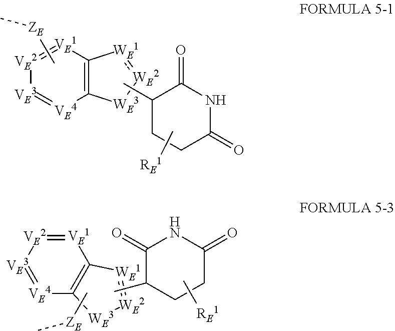

wherein

V_{E}¹, V_{E}², V_{E}³, and V_{E}⁴ are each independently selected from a bond, C, CR_{E}², and N; or V_{E}¹ and V_{E}², V_{E}² and V_{E}³, or V_{E}³ and V_{E}⁴ are combined together to optionally form 6 membered aryl ring or 5, 6 or 7 membered heteroaryl ring;

[symbol] indicates a single bond or a double bond; wherein (i) when there is a single bond between W_{E}¹ and W_{E}² (i.e. the [symbol] between W_{E}¹ and W_{E}² indicates single bond), W_{E}¹, W_{E}² and W_{E}³ are each independently selected from the group consisting of —N═, —CR_{E}³═, —CO—, —O—, —CR_{E}³R_{E}⁴—, —NR_{E}³—, —CR_{E}³═CR_{E}⁴—, —N═CR_{E}³—, and —N═N—; or (ii) when there is a double bond between W_{E}¹ and W_{E}² (i.e. the [symbol] between W_{E}¹ and W_{E}² indicates a double bond), W_{E}¹ and W_{E}² are each independently selected from the group consisting of —N═, —C≡ and —CR_{E}³═; W_{E}³ is selected from the group consisting of —CR_{E}³R_{E}⁴—, —O—, —N═, —NR_{E}³—, —C(O)NR_{E}³—, —CR_{E}³═CR_{E}⁴—, and —CR_{E}³═N—;

Z_{E}, R_{E}², R_{E}³, R_{E}⁴ and R_{E}⁵ are defined as in FORMULA 5.

In another embodiment, the degradation tag is a moiety of FORMULA 5-1 or 5-3, and wherein V_{E}¹, V_{E}², V_{E}³, and V_{E}⁴ are each independently selected from C, N, and CR_{E}².

In another embodiment, the degradation tag FORMULA 5-1 is a moiety of FORMULA 5A, 5B, 5E, 5F or 5G

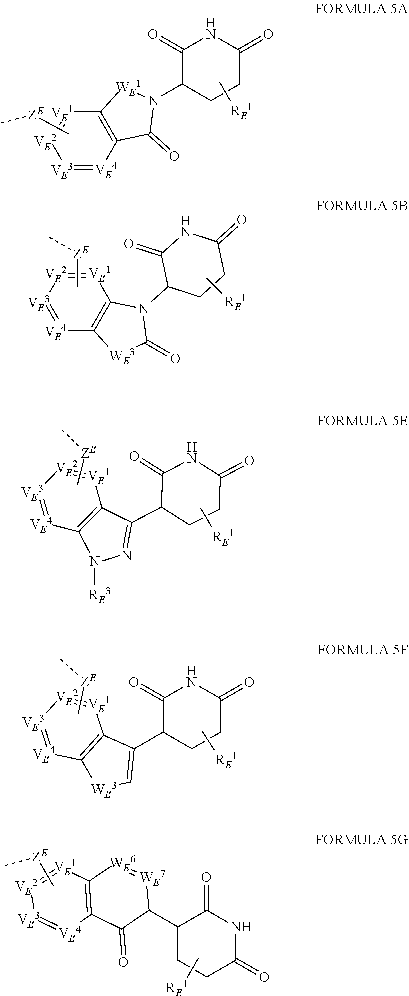

wherein W_{E}⁶ and W_{E}⁷ are each independently selected from —CR_{E}²═ and —N═; and V_{E}¹, V_{E}², V_{E}³, Y_{E}⁴, W_{E}¹, W_{E}³, Z_{E}, R_{E}³ and R_{E}¹ are defined as in FORMULA 5-1.

In another embodiment, the degradation tag is a moiety of FORMULA 5A, 5B, 5E, 5F or 5G, and wherein V_{E}¹, V_{E}², V_{E}³, and V_{E}⁴ are each independently selected from a bond, C, CR_{E}² and N (preferably, C, CR_{E}² and N).

In another embodiment, the degradation tag is a moiety of FORMULA 5A, 5B, 5E, 5F or 5G, and wherein W_{E}¹ and W_{E}³ are each independently selected from —CO—, —O—, —CR_{E}³R_{E}⁴—, —NR_{E}³—, —CR_{E}³═CR_{E}⁴—, —N═CR_{E}³—, and —N═N—; preferably, W_{E}¹ and W_{E}³ are each independently selected from —CO—, —O—, —CR_{E}³R_{E}⁴—, and —NR_{E}³—.

In another embodiment, the degradation tag FORMULA 5-3 is moiety of FORMULA 5C

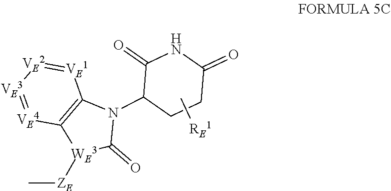

wherein W_{E}³ is N or CR_{E}³; and V_{E}¹, V_{E}², V_{E}³, V_{E}⁴, Z_{E}, and R_{E}¹ are defined as in FORMULA 5-3. In another embodiment, the degradation tag is a moiety of FORMULA 5C, wherein V_{E}¹, V_{E}², V_{E}³, and V_{E}⁴ are each independently selected from a bond, CR_{E}² and N.

In another embodiment, the degradation tag is a moiety of FORMULA 5-2,

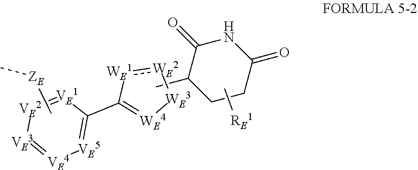

V_{E}¹, V_{E}², V_{E}³, V_{E}⁴ and V_{E}⁵ are each independently selected from a bond, C, CR_{E}², and N; or V_{E}¹ and V_{E}², V_{E}² and V_{E}³, V_{E}³ and V_{E}⁴, or V_{E}⁴ and V_{E}⁵ are combined together to optionally form C₆ aryl ring or 5, 6, or 7 heteroaryl ring;

[symbol] indicates a single bond or a double bond; (i) when there is a single bond between W_{E}¹ and W_{E}² (i.e. the [symbol] between W_{E}¹ and W_{E}² indicates single bond), W_{E}¹ and W_{E}⁴ are each independently selected from —N═, —CR_{E}³═, —CO—, —O—, —CR_{E}³R_{E}⁴—, —NR_{E}³—, —CR_{E}³═CR_{E}⁴—, —N═CR_{E}³—, and —N═N—, and W_{E}² and W_{E}³ are each independently selected from —N═, —CR_{E}³═, —CO—, —O—, —CR_{E}³R_{E}⁴—, and —NR_{E}³—; or (ii) when there is a double bond between W_{E}¹ and W_{E}² (i.e. the [symbol] between W_{E}¹ and W_{E}² indicates a double bond), W_{E}¹ and W_{E}² are each independently selected from —N═, C and —CR_{E}²═; W_{E}³ is selected from —N═, —CR_{E}³═, —CO—, —O—, —CR_{E}³R_{E}⁴—, and —NR_{E}³—; and W_{E}⁴ is selected from —N═, —CR_{E}³═, —CO—, —O—, —CR_{E}³R_{E}⁴—, —NR_{E}³—, —CR_{E}³═CR_{E}⁴—, —N═CR_{E}³—, and —N═N—;

Z_{E}, R_{E}², R_{E}³, R_{E}⁴ and R_{E}¹ are defined as in FORMULA 5.

In another embodiment, the degradation tag is a moiety of FORMULA 5-2, wherein V_{E}¹, V_{E}², V_{E}³, V_{E}⁴ and V_{E}⁵ are each independently selected from a bond, C, CR_{E}², and N.

In another embodiment, the degradation tag is a moiety of FORMULA 5-2, wherein [symbol] indicates a single bond.

In another embodiment, the degradation tag is a moiety of FORMULA 5-2, wherein [symbol] indicates a single bond, W_{E}¹ and W_{E}⁴ are each independently selected from —CO—, —O—, —CR_{E}³R_{E}⁴—, and —NR_{E}³—, and W_{E}² and W_{E}³ are each independently selected from —N═, —CR_{E}³═, —CO—, —O—, —CR_{E}³R_{E}⁴—, and —NR_{E}³—.

In another embodiment, the degradation tag FORMULA 5-2 is moiety of FORMULA 5D.

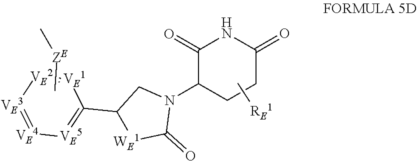

wherein V_{E}¹, V_{E}², V_{E}³, V_{E}⁴, V_{E}⁵, W_{E}¹, Z_{E}, and R_{E}¹ are defined as in FORMULA 5-2.

In another embodiment, the degradation tag is a moiety of FORMULA 5D, wherein W_{E}¹ is selected from —CO—, —O—, —CR_{E}³R_{E}⁴—, —NR_{E}³—, —CR_{E}³═CR_{E}⁴—, —N═CR_{E}³—, and —N═N—; preferably, W_{E}¹ is selected from —CO—, —O—, —CR_{E}³R_{E}⁴—, and —NR_{E}³—.

In another embodiment, the degradation tag is a moiety of FORMULA 5D, wherein V_{E}¹, V_{E}², V_{E}³, V_{E}⁴, and V_{E}⁵ are each independently selected from a bond, C, CR_{E}² and N; or V_{E}¹ and V_{E}², V_{E}² and V_{E}³, V³ and V_{E}⁴, or V_{E}⁴ and V_{E}⁵ are combined together to optionally form a C₆ aryl ring or 5, 6 or 7 heteroaryl ring; preferably, V_{E}¹, V_{E}², V_{E}³, V_{E}⁴, and V_{E}⁵ are each independently selected from a bond, C, CR_{E}² and N.

In another embodiment, the degradation tag is a moiety of FORMULA 5-4,

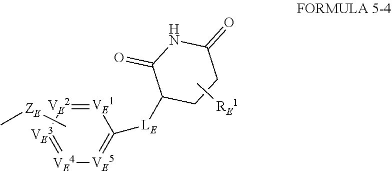

wherein V_{E}¹, V_{E}², V_{E}³, V_{E}⁴, V_{E}⁵. L_{E}, Z_{E}, and R_{E}¹ are defined as in FORMULA 5.

In another embodiment, the degradation tag is a moiety of FORMULA 5-4, and wherein L_{E} is not null.

In another embodiment, the degradation tag is a moiety of FORMULA 5-4, and wherein L_{E} is selected from the group consisting of —NH—, —N(C₁-C₄ alkyl)-, —CO—, —NH—CO—, —N(C₁-C₄ alkyl)-CO—, —CO—NH—, and —CO—N(C₁-C₄ alkyl)-.

In another embodiment, the degradation tag is a moiety of FORMULA 5-4, and wherein

V_{E}¹, V_{E}², V_{E}³, V_{E}⁴ and V_{E}⁵, at each occurrence, are each independently selected from the group consisting of C, CR_{E}² and N; or

V_{E}¹ and V_{E}², V_{E}² and V_{E}³, V_{E}³ and V_{E}⁴; or V_{E}⁴ and V_{E}⁵ are combined together to optionally form a ring of

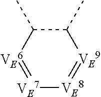

wherein V_{E}⁶, V_{E}⁷, V_{E}⁸, and V_{E}⁹ are each independently selected from the group consisting of C, CR_{E}¹² and N;

R_{E}¹², at each occurrence, is independently selected from the group consisting of hydrogen, halogen, cyano, nitro, hydroxy, amino, optionally substituted C₁-C₆ alkyl, optionally substituted C₁-C₆ alkenyl, optionally substituted C₁-C₆ alkynyl, optionally substituted C₁-C₆ alkoxy, optionally substituted C₁-C₆ alkylamino, optionally substituted 3-8 membered carbocyclyl, and optionally substituted 3-8 membered heterocyclyl.

In another embodiment, the degradation tag is a moiety of FORMULA 5-4, and wherein V_{E}⁶, V_{E}⁷, V_{E}⁸, and V_{E}⁹ are each independently selected from the group consisting of CR_{E}¹² and N.

In another embodiment, the degradation tag is a moiety of FORMULA 5-4, and wherein R_{E}¹², at each occurrence, is independently selected from the group consisting of hydrogen, halogen, cyano, nitro, hydroxy, amino, optionally substituted C₁-C₆ alkyl.

In another embodiment, the degradation tag is a moiety of FORMULA 5-4, and wherein

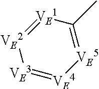

is selected from the group consisting of

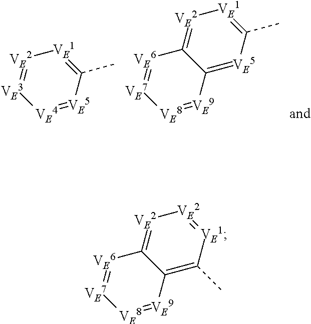

wherein

V_{E}¹, V_{E}², V_{E}³, V_{E}⁴ and V_{E}⁵ are each independently selected from the group consisting of C, CR_{E}² and N; and V_{E}⁶, V_{E}⁷, V_{E}⁸, and V_{E}⁹ are each independently selected from the group consisting of CR_{E}¹² and N.

In another embodiment, the degradation tag is a moiety of FORMULA 5-4, and wherein Z_{E} is null, —CH₂—, —O—, or —NH—.

In another embodiment, the degradation tag FORMULA 5-4 is moiety of FORMULA 5H or 5I:

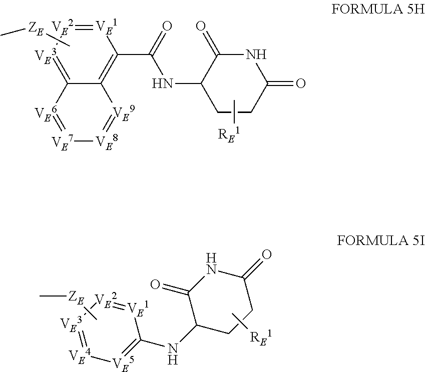

wherein V_{E}¹, V_{E}², V_{E}³, V_{E}⁴, V_{E}⁵, V_{E}⁶, V_{E}⁷, V_{E}⁸, and V_{E}⁹ are each independently selected from a bond, C, CR_{E}² and N; and Z_{E} and R_{E}¹ are defined as in FORMULA 5-4.

In another embodiment, the degradation tag is a moiety of FORMULA 5-4, and wherein L_{E} is null.

In another embodiment, the degradation tag FORMULA 5-4 is moiety of FORMULA 5N;

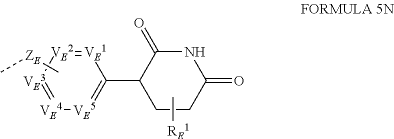

wherein V_{E}¹, V_{E}², V_{E}³, V_{E}⁴, and V_{E}⁵ are each independently selected from a bond, C, CR_{E}² and N; and Z_{E} and R_{E}¹ are defined as in FORMULA 5-4.

In another embodiment, the degradation tag is a moiety of FORMULA 5-5,

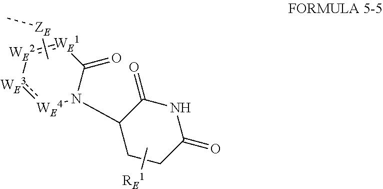

wherein [symbol] , W_{E}¹, W_{E}², W_{E}³, W_{E}⁴, Z_{E} and R_{E}¹ are defined as in FORMULA 5.

In another embodiment, the degradation tag is a moiety of FORMULA 5-5, and wherein W_{E}¹, W_{E}², W_{E}³ and W_{E}⁴ are each independently selected from the group consisting of —N═, —C—, —CR_{E}³═, —CO—, —O—, —CR_{E}³R_{E}⁴—, and —NR_{E}³—.

In another embodiment, the degradation tag is a moiety of FORMULA 5-5, and wherein W_{E}¹, W_{E}², W_{E}³ and W_{E}⁴ are each independently selected from the group consisting of —N═, —C—, —CH═, —CO—, —O—, —N—, —CH₂—, and —NH—.

In another embodiment, the degradation tag FORMULA 5-5 is moiety of FORMULA 5J, 5K or 5L;

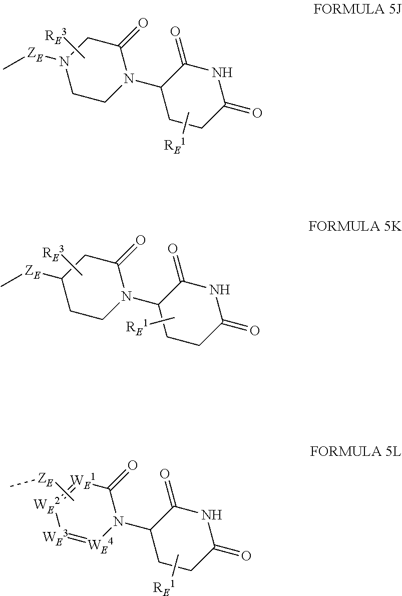

wherein W_{E}¹, W_{E}², W_{E}³, W_{E}⁴, Z_{E}, R_{E}³ and R_{E}¹ are defined as in FORMULA 5-5.

In another embodiment, the degradation tag is a moiety of FORMULA 5-6,

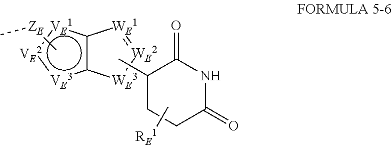

wherein

V_{E}¹, V_{E}², and V_{E}³ are each independently selected from C, CR_{E}², S, N, and NR_{E}²; or V_{E}¹ and V_{E}², or V_{E}² and V_{E}³ are combined together to optionally form 5 membered heteroaryl ring;

[symbol] indicates a single bond or a double bond; wherein (i) when there is a single bond between W_{E}¹ and W_{E}² (i.e. the [symbol] between W_{E}¹ and W_{E}² indicates single bond), W_{E}¹, W_{E}² and W_{E}³ are each independently selected from the group consisting of —N═, —CR_{E}³═, —CO—, —O—, —CR_{E}³R_{E}⁴—, —NR_{E}³—, —CR_{E}³═CR_{E}⁴—, —N═CR_{E}³—, and —N═N—; or (ii) when there is a double bond between W_{E}¹ and W_{E}² (i.e. the [symbol] between W_{E}¹ and W_{E}² indicates a double bond), W_{E}¹ and W_{E}² are each independently selected from the group consisting of —N—, — [symbol] C≡ and —CR_{E}³═; W_{E}³ is selected from the group consisting of —O—, —N═, —NR_{E}³—, —C(O)NR_{E}³—, —CR_{E}³R_{E}⁴—, —CR_{E}³═CR_{E}⁴—, and —CR_{E}³═N—;

Z_{E}, R_{E}², R_{E}³, R_{E}⁴ and R_{E}¹ are defined as in FORMULA 5.

In another embodiment, the degradation tag is a moiety of FORMULA 5-6, and wherein V_{E}¹, V_{E}², V_{E}³, and V_{E}⁴ are each independently selected from C, CR_{E}², S, N, and NR_{E}².

In another embodiment, the degradation tag FORMULA 5-6 is moiety of FORMULA 5M:

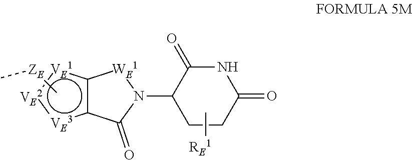

wherein V_{E}¹, V_{E}², V_{E}³, W_{E}⁴, Z_{E} and R_{E}¹ are defined as in FORMULA 5-6.

In another embodiment, the degradation tag is a moiety of FORMULA 5M, and wherein V_{E}¹, V_{E}², and V_{E}³ are each independently selected from C, CR_{E}², S, N, and NR_{E}² (preferably, one of V_{E}¹, V_{E}², and V_{E}³ is S).

In another embodiment, the degradation tag is a moiety of FORMULA 5M, and wherein W_{E}¹ is selected from —CO—, —O—, —CR_{E}³R_{E}⁴—, —NR_{E}³—, —CR_{E}³═CR_{E}⁴—, —N═CR_{E}³—, and —N═N—; preferably, W_{E}¹ is selected from —CO—, —O—, —CR_{E}³R_{E}⁴—, and —NR_{E}³—.

In another embodiment, the degradation tag is a moiety of FORMULA 5-7,

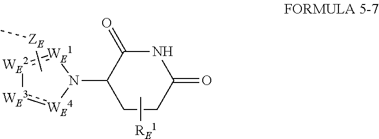

wherein W_{E}¹, W_{E}², W_{E}³, W_{E}⁴, Z_{E}, and R_{E}¹ are defined as in FORMULA 5.

In another embodiment, the degradation tag is a moiety of FORMULA 5-7, and wherein [symbol] is double bond;

W_{E}¹ and W_{E}², are combined together to optionally form a ring of

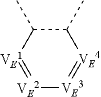

wherein V_{E}¹, V_{E}², V_{E}³, and V_{E}⁴ are each independently selected from the group consisting of C, CR_{E}¹² and N;

W_{E}³ and W_{E}⁴, are combined together to optionally form a ring of

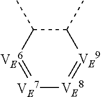

wherein V_{E}⁶, V_{E}⁷, V_{E}⁸, and V_{E}⁹ are each independently selected from the group consisting of C, CR_{E}¹² and N; and

R_{E}¹², at each occurrence, is independently selected from the group consisting of hydrogen, halogen, cyano, nitro, hydroxy, amino, optionally substituted C₁-C₆ alkyl, optionally substituted C₁-C₆ alkenyl, optionally substituted C₁-C₆ alkynyl, optionally substituted C₁-C₆ alkoxy, optionally substituted C₁-C₆ alkylamino, optionally substituted 3-8 membered carbocyclyl, and optionally substituted 3-8 membered heterocyclyl.

In another embodiment, the degradation tag is a moiety of FORMULA 5-7, and wherein R_{E}¹², at each occurrence, is independently selected from the group consisting of hydrogen, halogen, cyano, nitro, hydroxy, amino, optionally substituted C₁-C₆ alkyl.

In another embodiment, the degradation tag FORMULA 5-7 is moiety of FORMULA 5O;

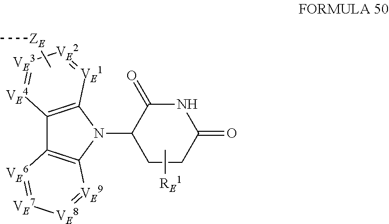

wherein V_{E}¹, V_{E}², V_{E}³, V_{E}⁴, V_{E}⁵, V_{E}⁶, V_{E}⁷, V_{E}⁸, and V_{E}⁹ are each independently selected from C, CR_{E}² and N; and Z_{E} and R_{E}¹ are defined as in FORMULA 5-7.

In another embodiment, the degradation tag is a moiety of FORMULA 5-8,

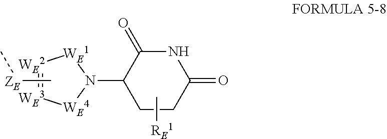

wherein W_{E}¹, W_{E}², W_{E}³, W_{E}⁴, Z_{E} and R_{E}¹ are defined as in FORMULA 5.

In another embodiment, the degradation tag is a moiety of FORMULA 5-8, and [symbol] is a double bond.

In another embodiment, the degradation tag is a moiety of FORMULA 5-8, and W_{E}¹ and W_{E}⁴ are each independently selected from the group consisting of —CO—, —O—, or —CR_{E}³R_{E}⁴—.

In another embodiment, the degradation tag is a moiety of FORMULA 5-8, and W_{E}² is arylamino or heteroarylamino.

In another embodiment, the degradation tag is a moiety of FORMULA 5-8, and W_{E}³ is CR_{E}³ or N.

In another embodiment, the degradation tag FORMULA 5-8 is moiety of FORMULA 5P;

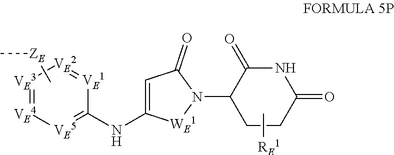

wherein V_{E}¹, V_{E}², V_{E}³, V_{E}⁴, and V_{E}⁵ are each independently selected from C, CR_{E}² and N; W_{E}¹ is selected from CO, CH₂, and O; and Z_{E} and R_{E}¹ are defined as in FORMULA 5.

In another preferred embodiment, the degradation tag is a moiety of FORMULA 5A.

In another preferred embodiment, the degradation tag is a moiety of FORMULA 5A and Z_{E} is connected to V_{E}¹ or V_{E}⁴.

In another embodiment, the degradation tag is a moiety of FORMULAE 8A to 8HT:

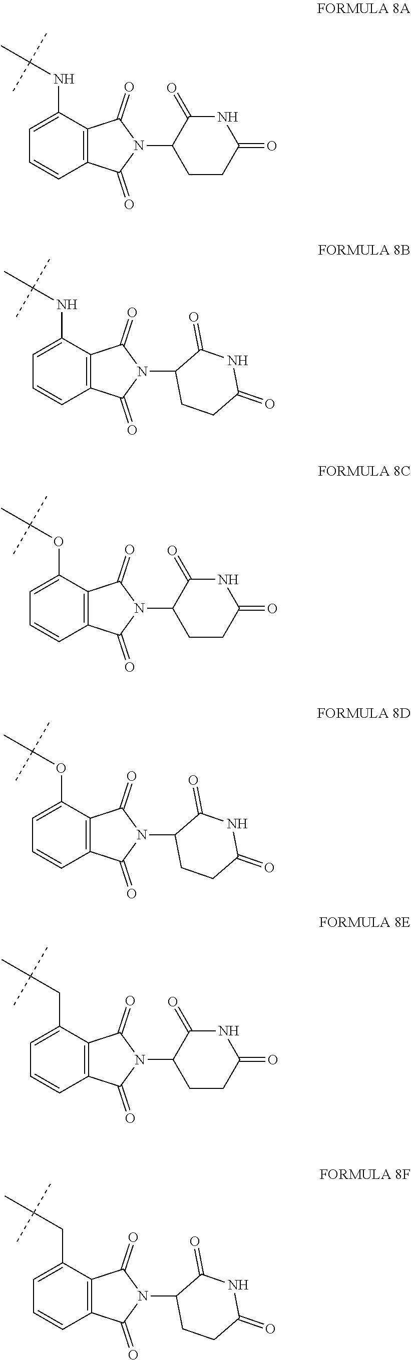

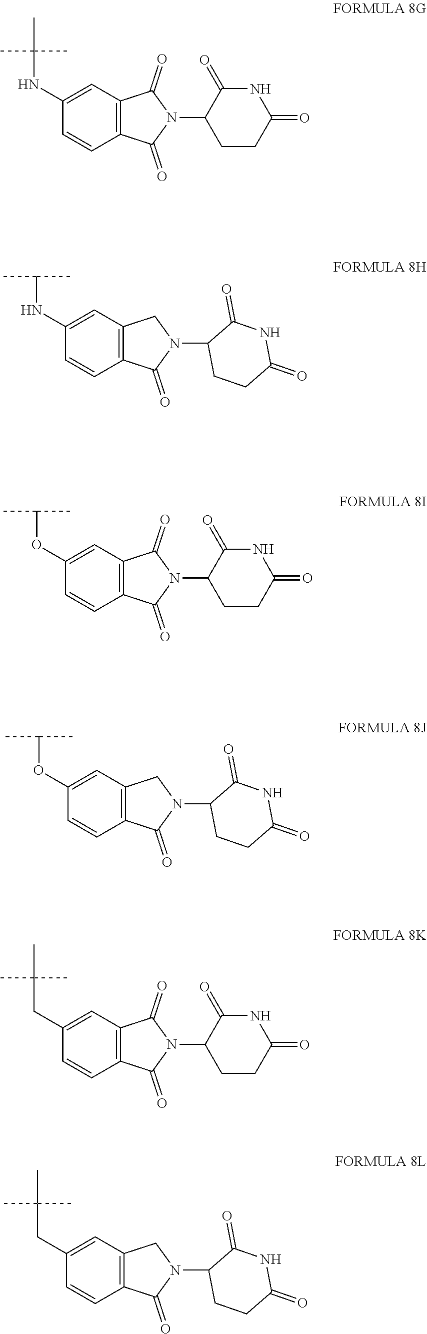

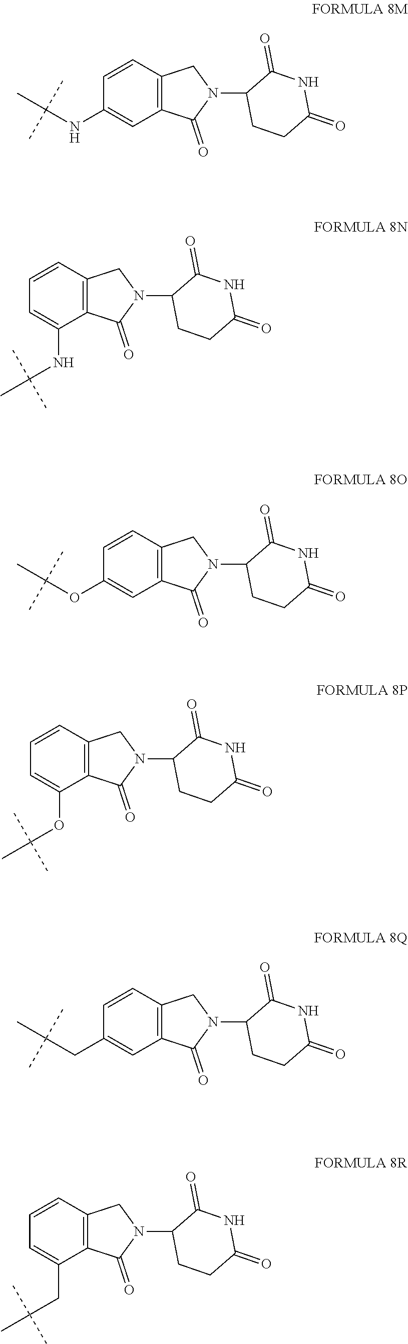

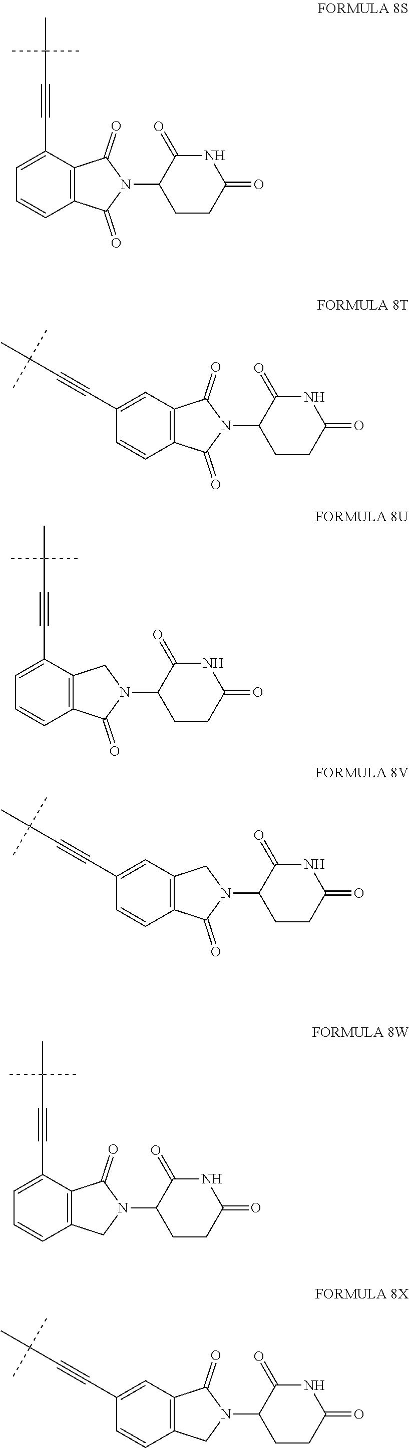

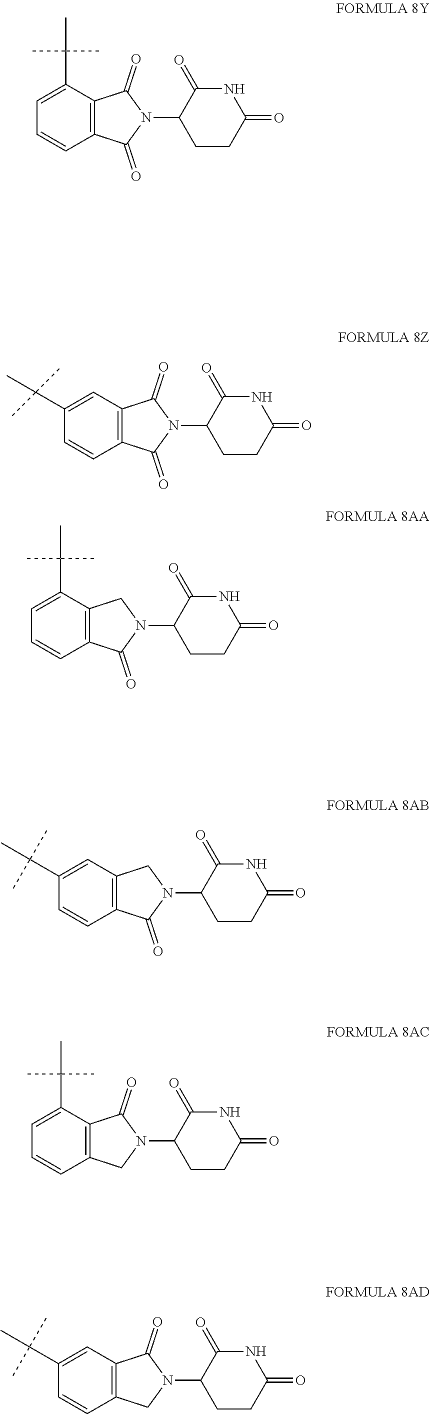

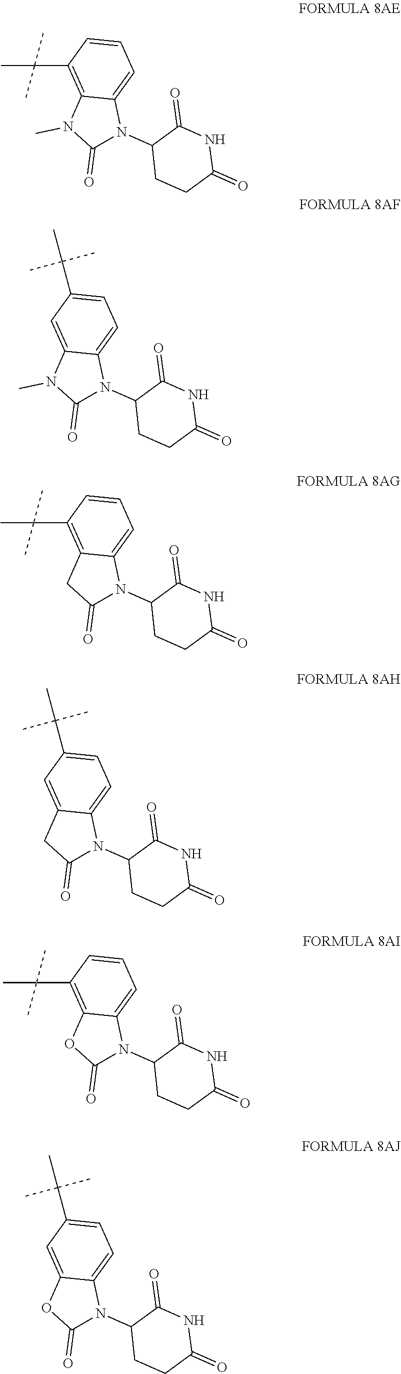

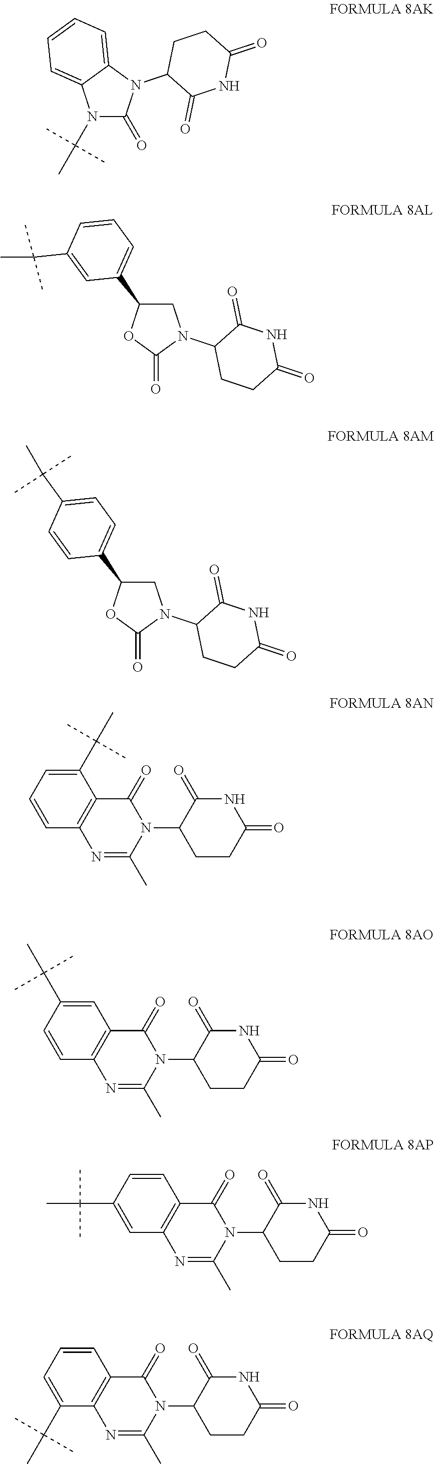

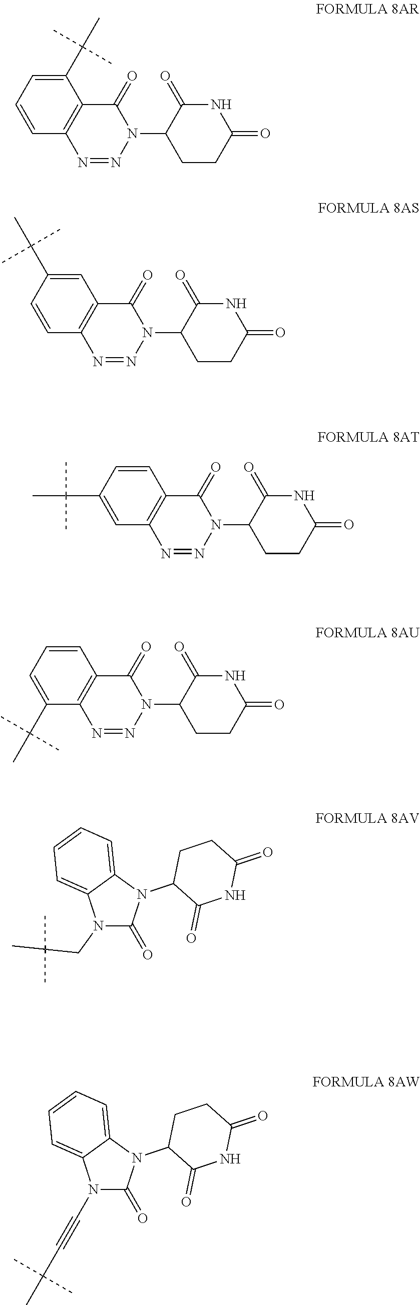

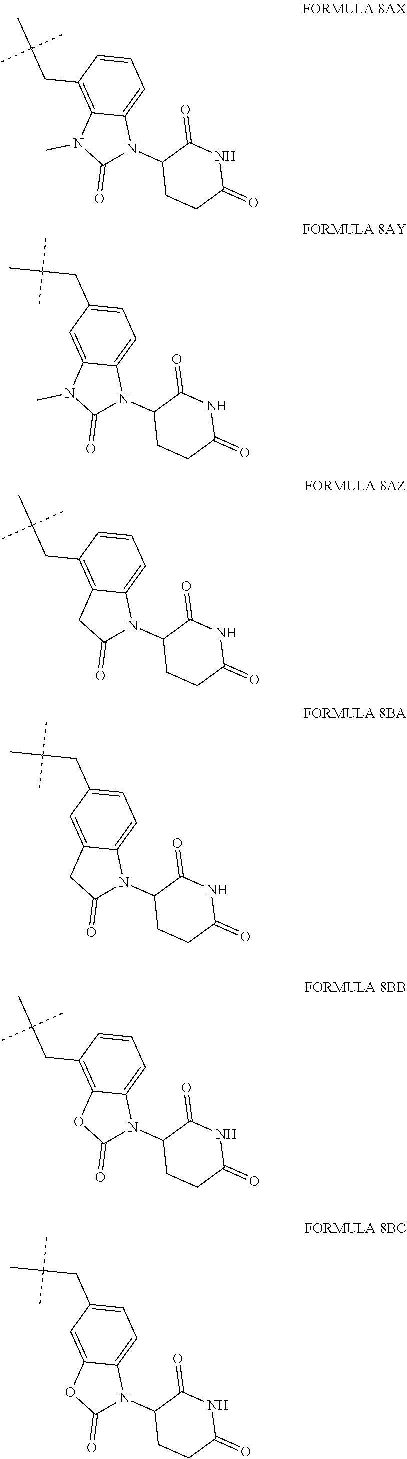

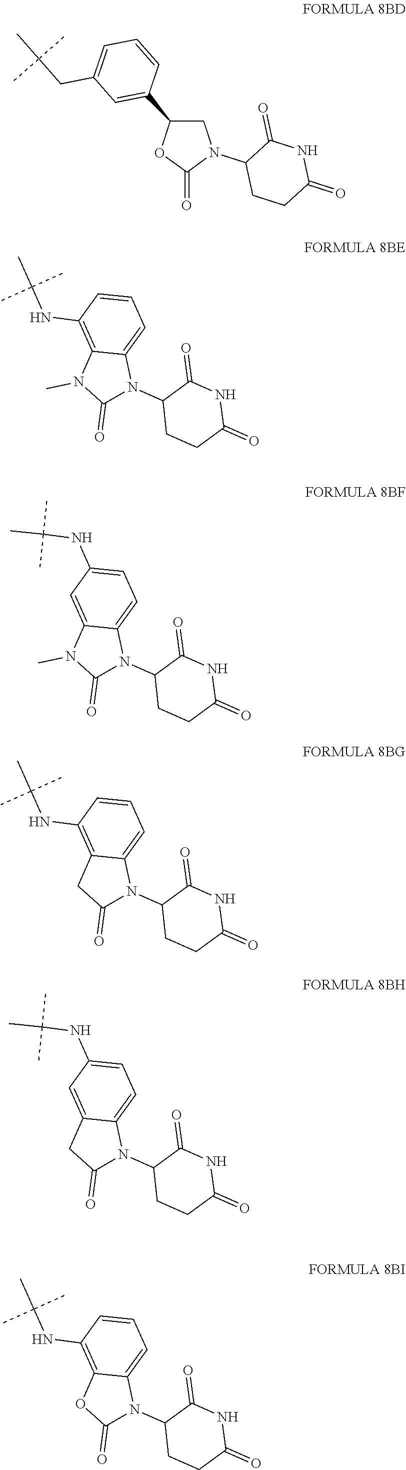

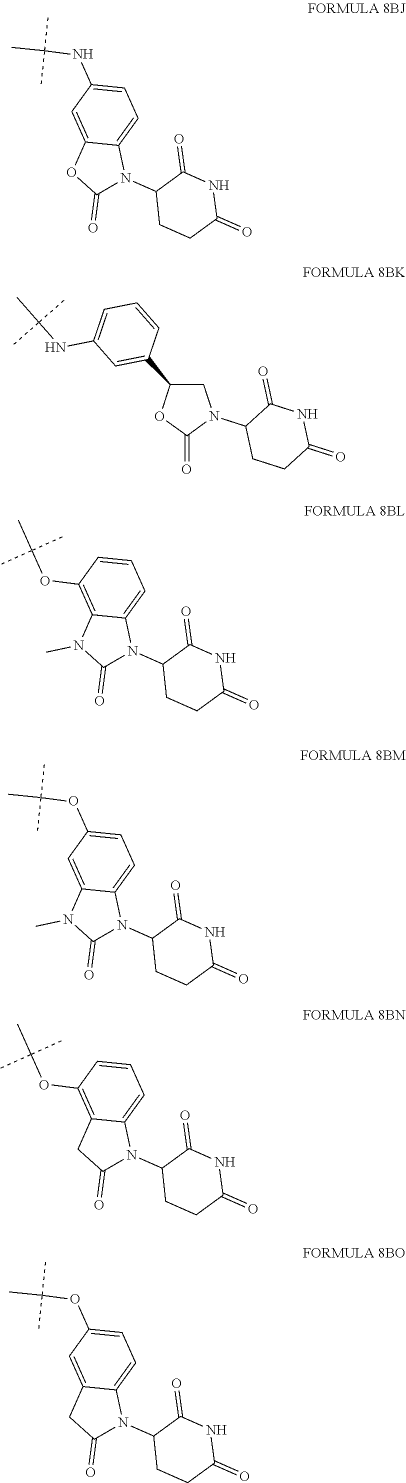

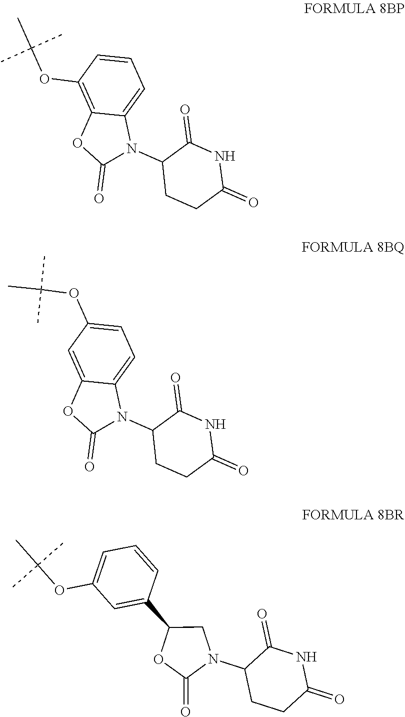

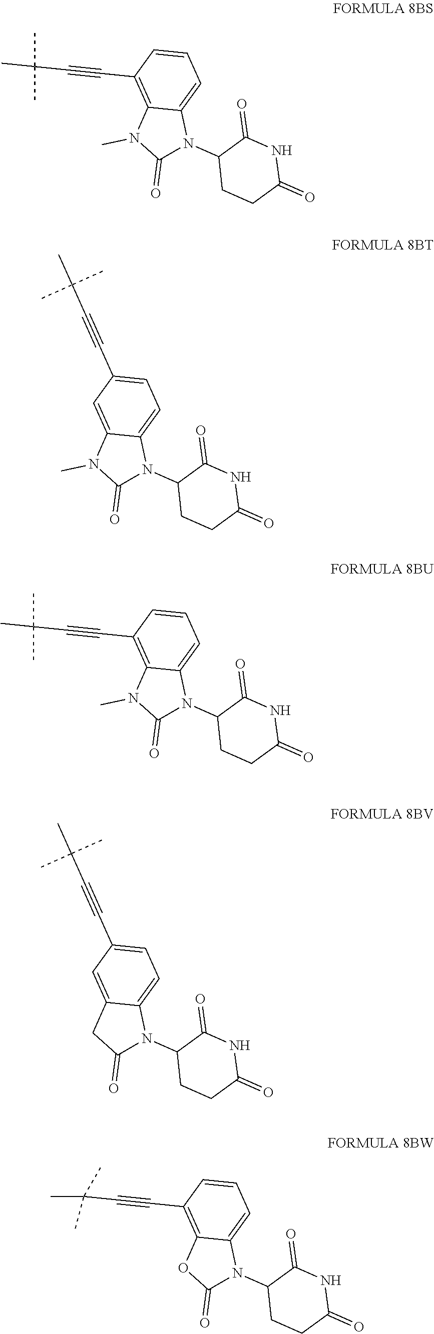

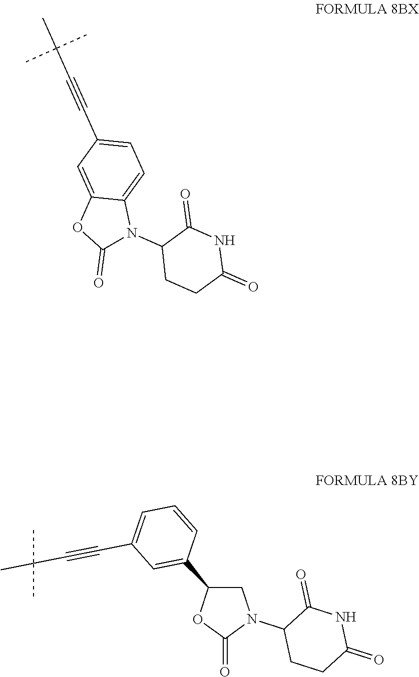

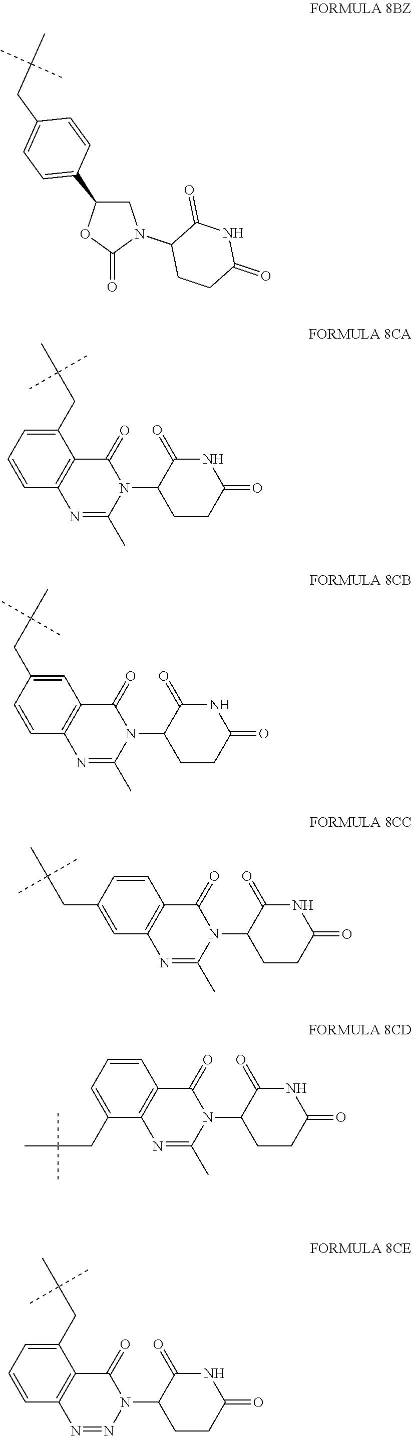

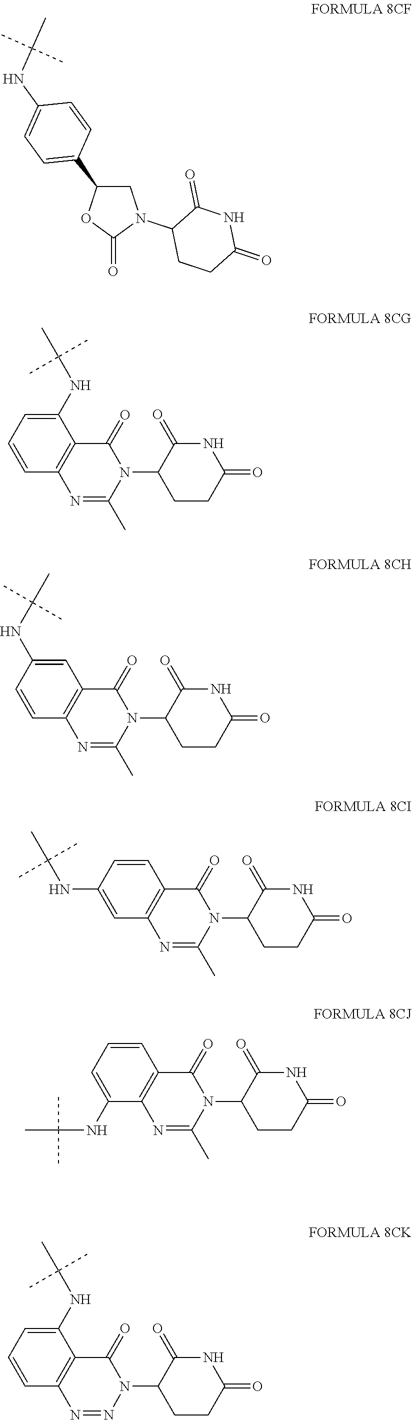

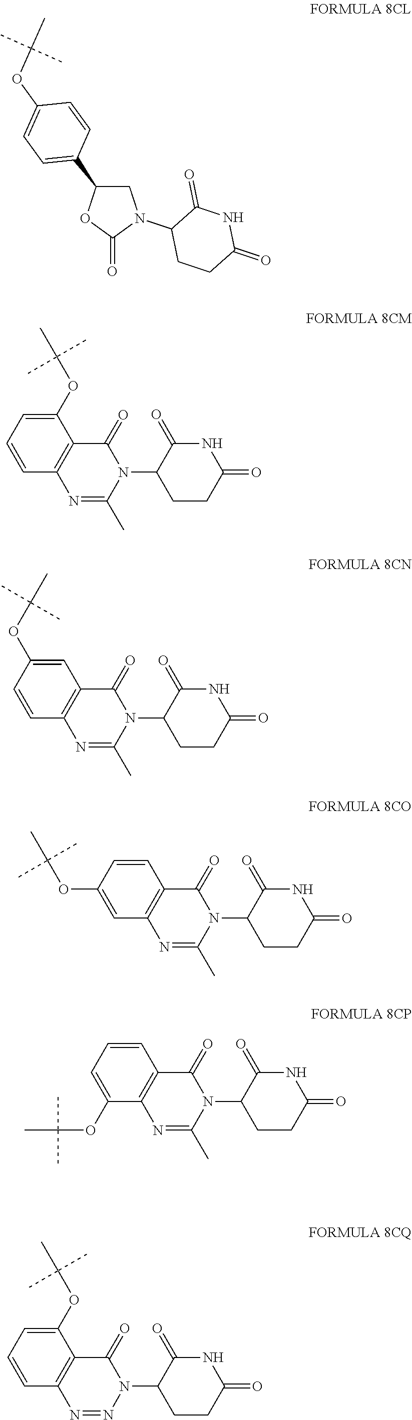

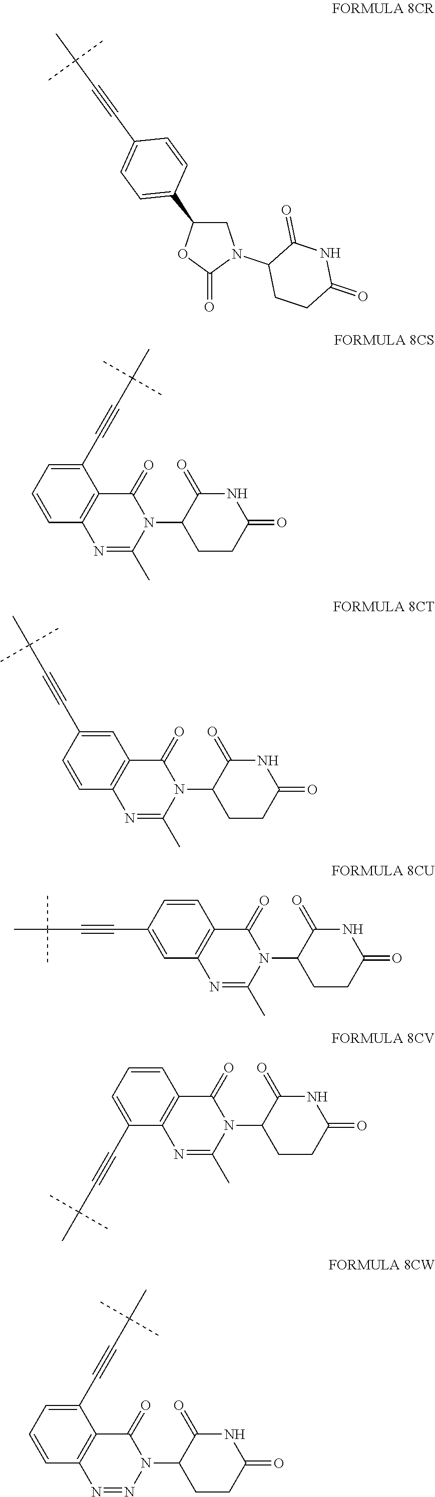

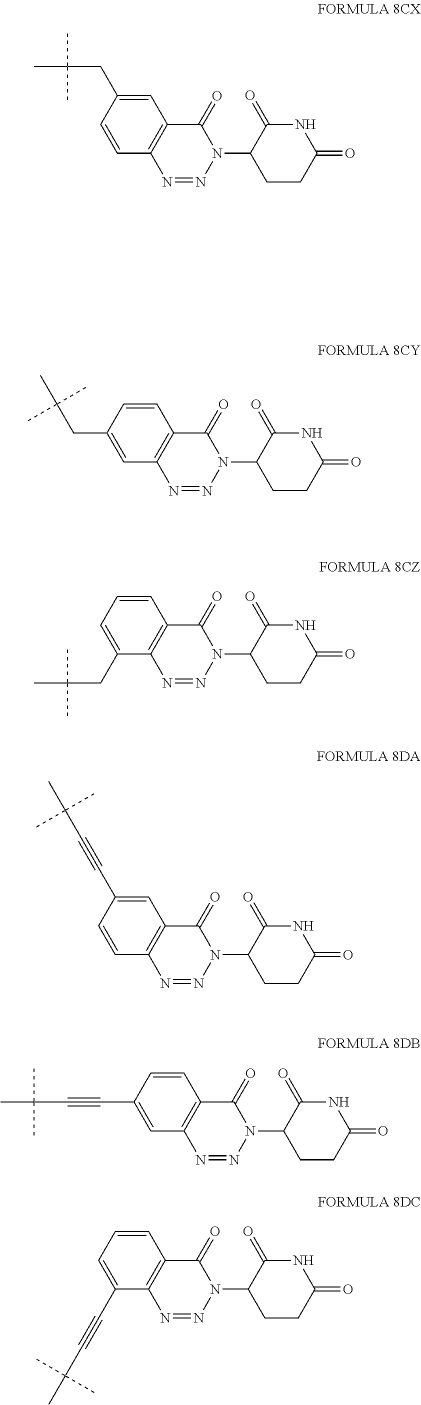

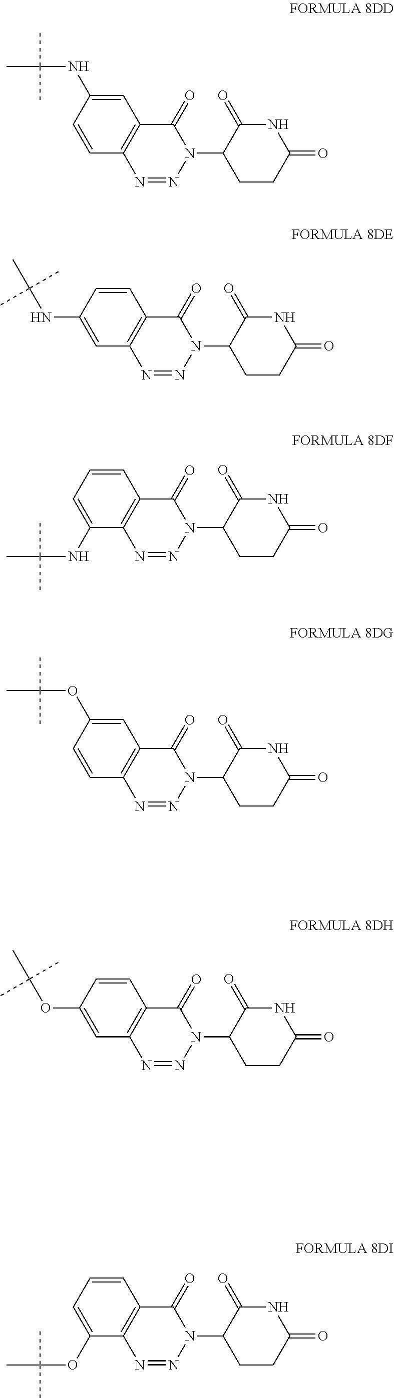

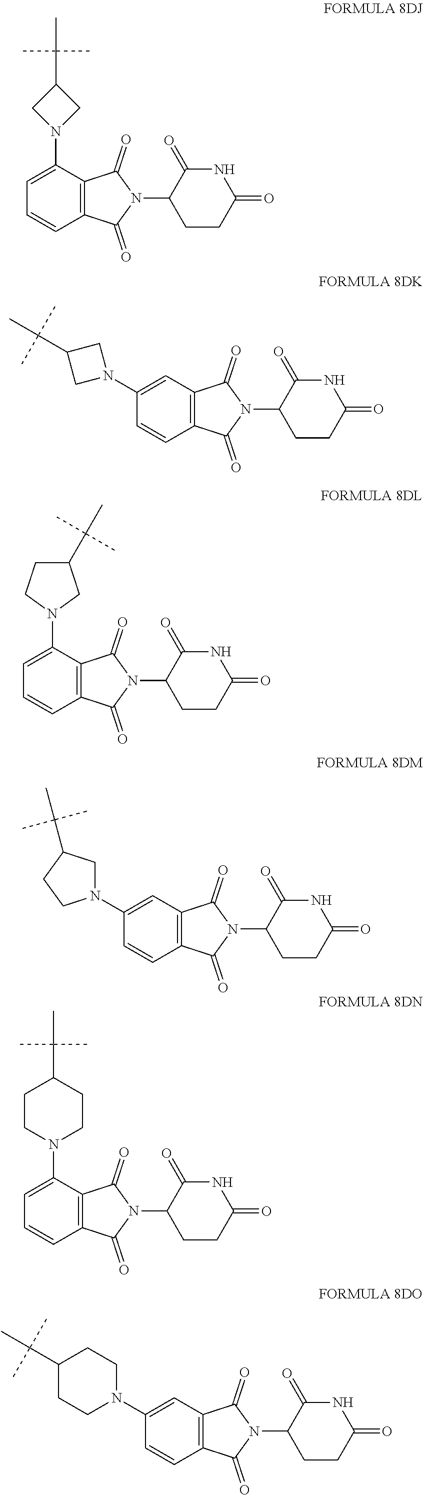

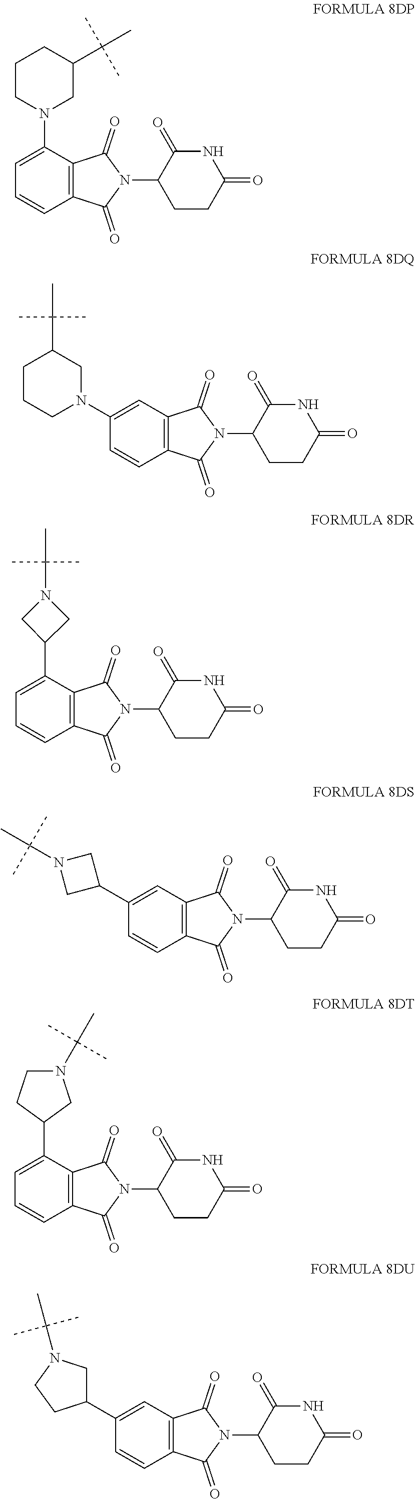

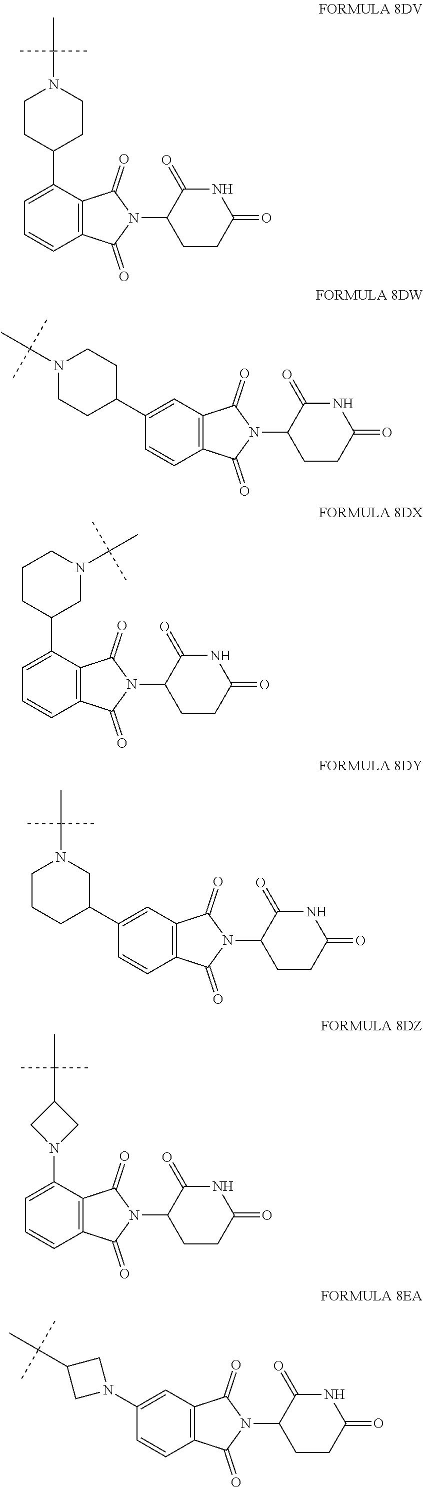

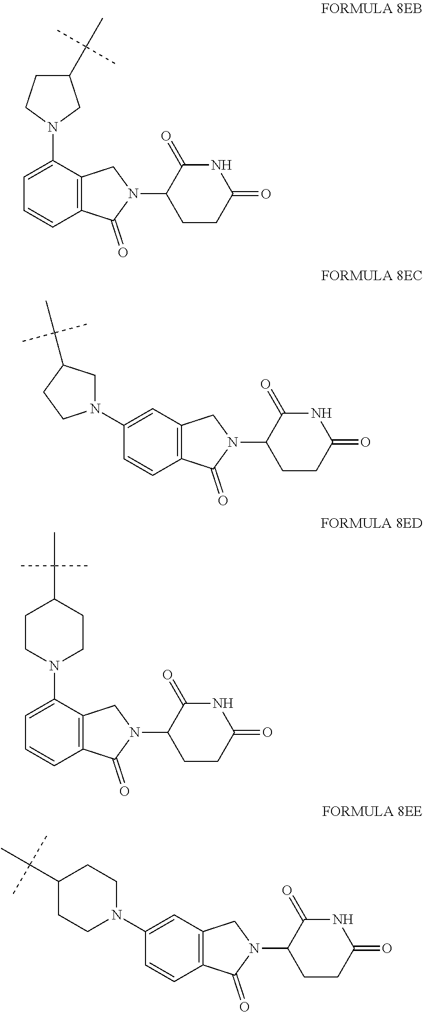

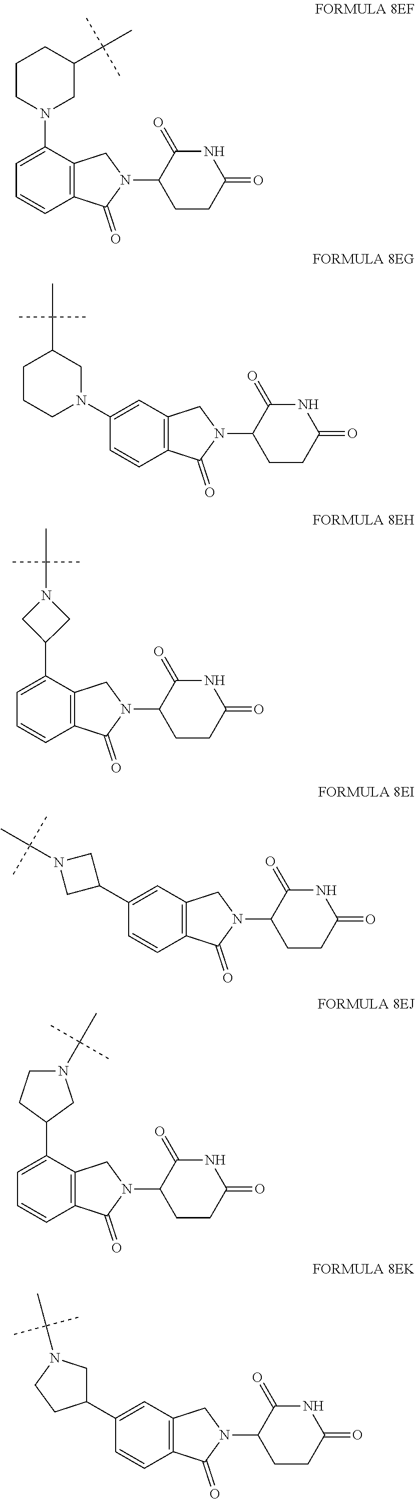

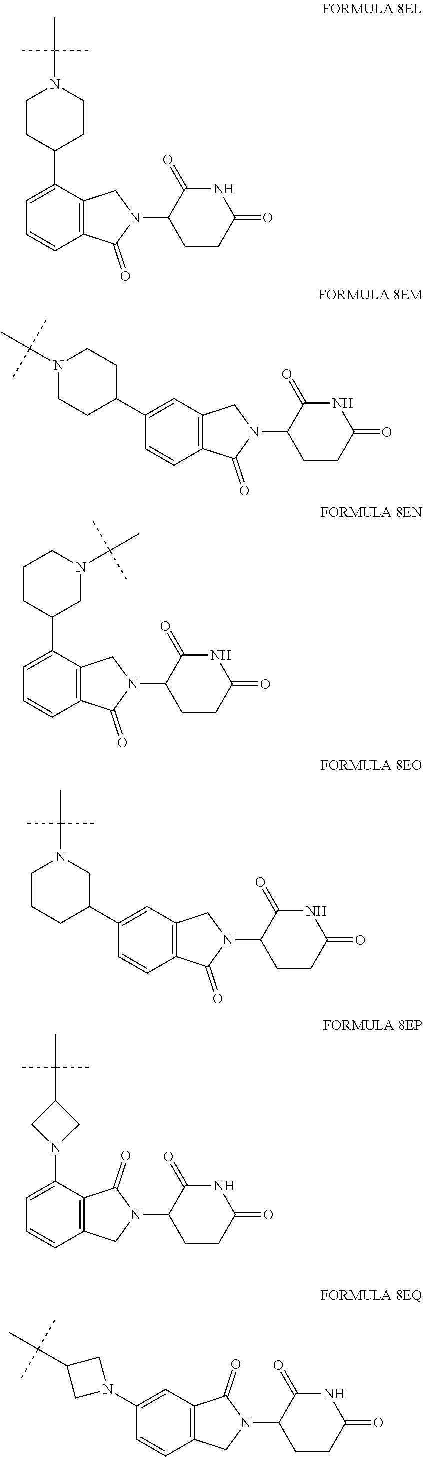

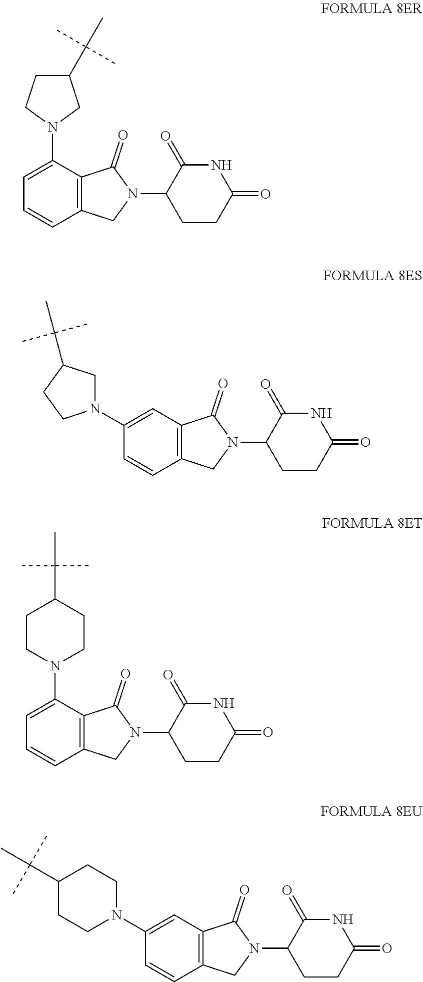

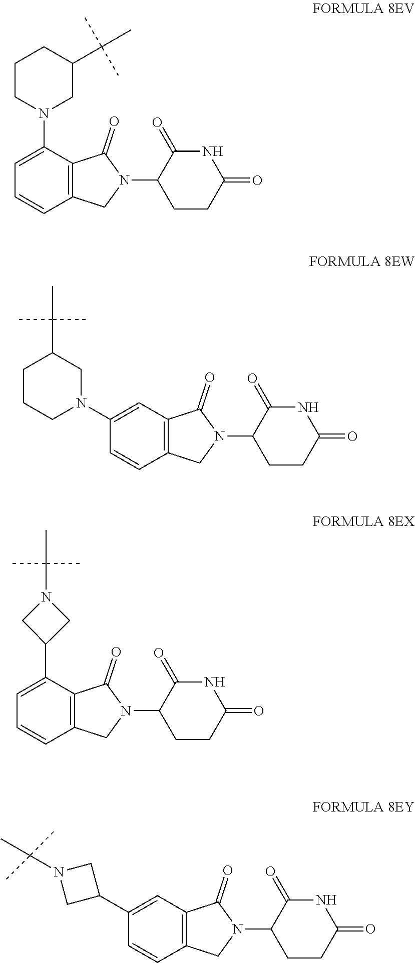

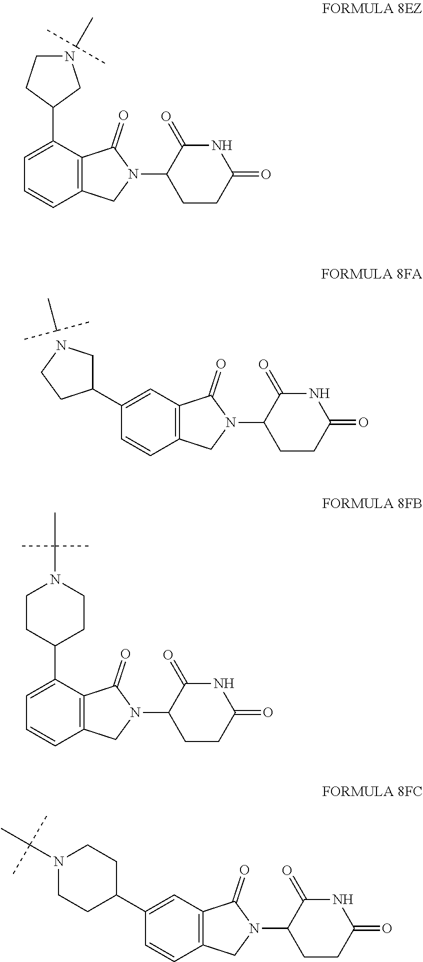

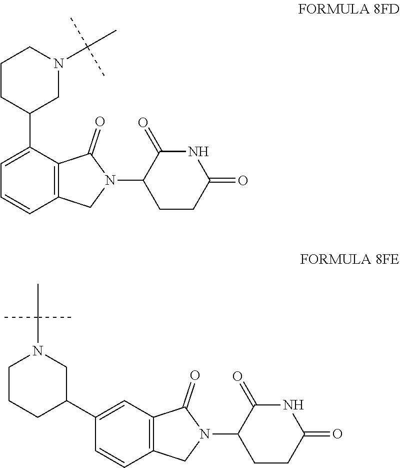

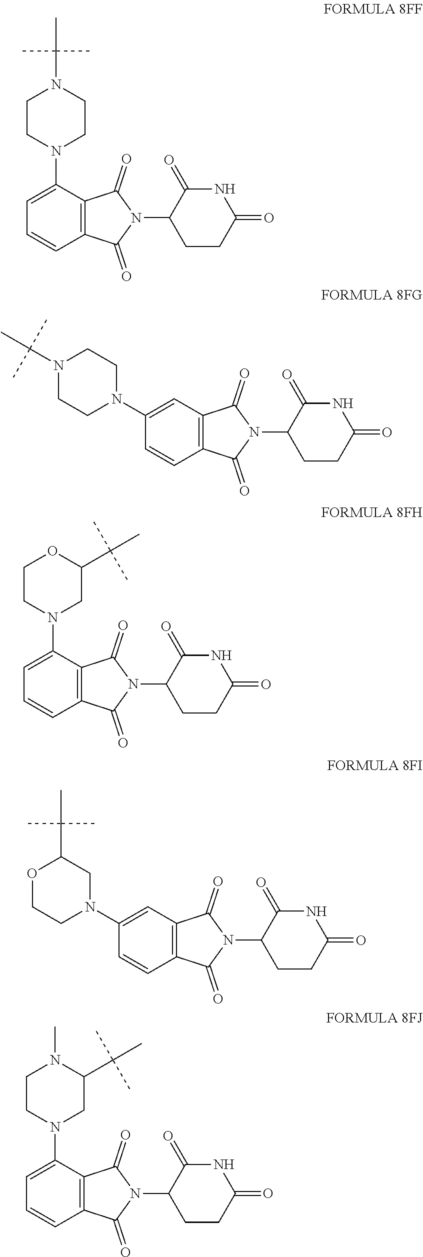

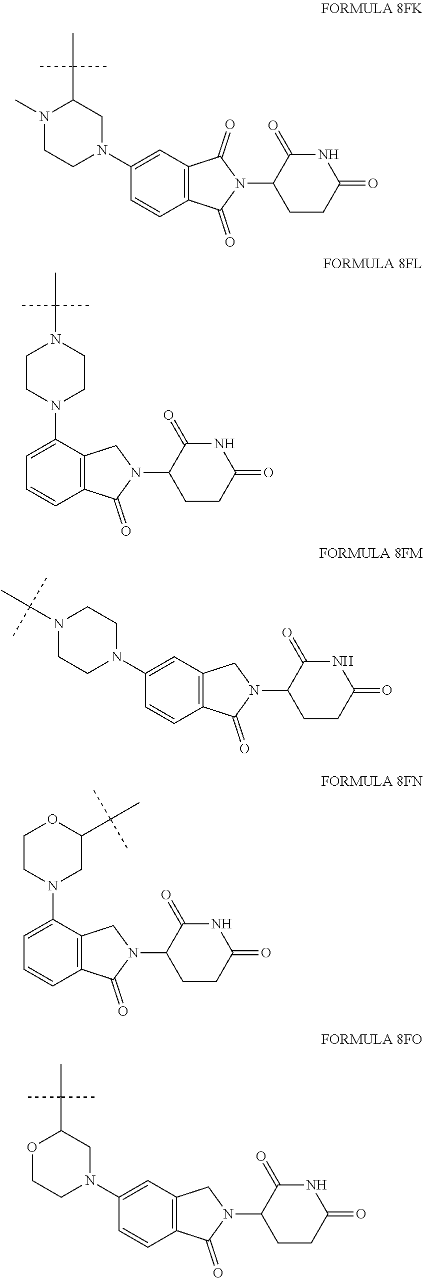

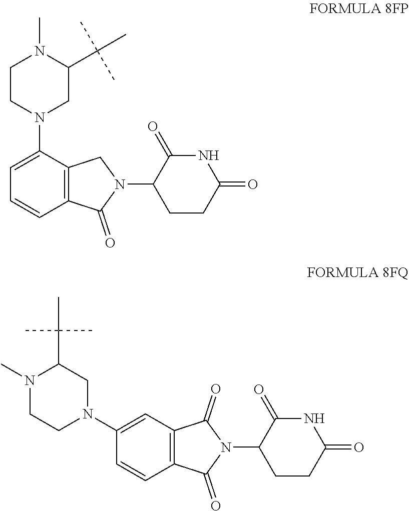

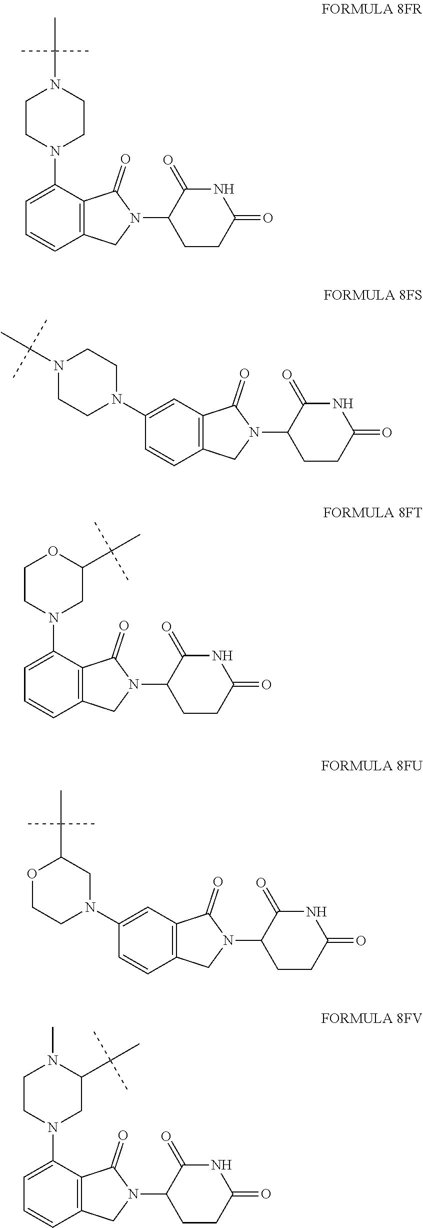

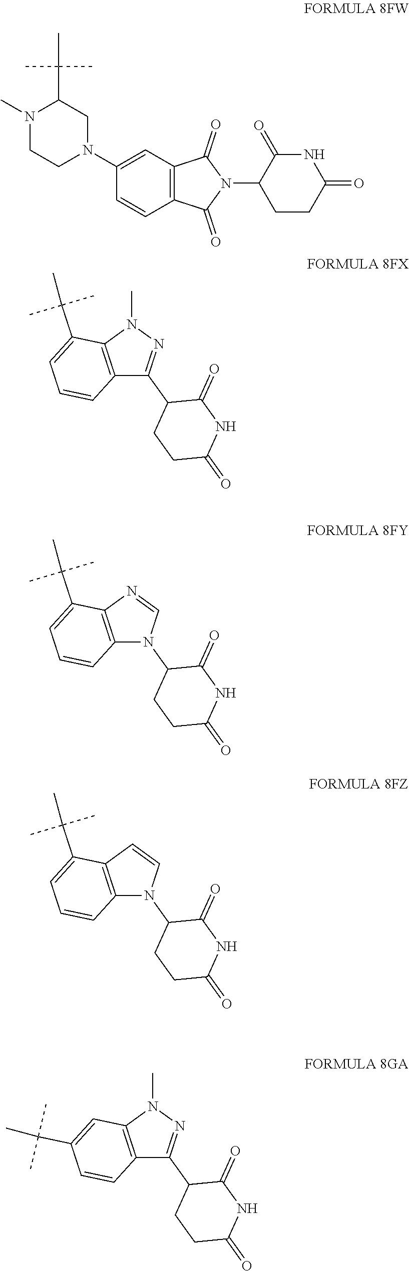

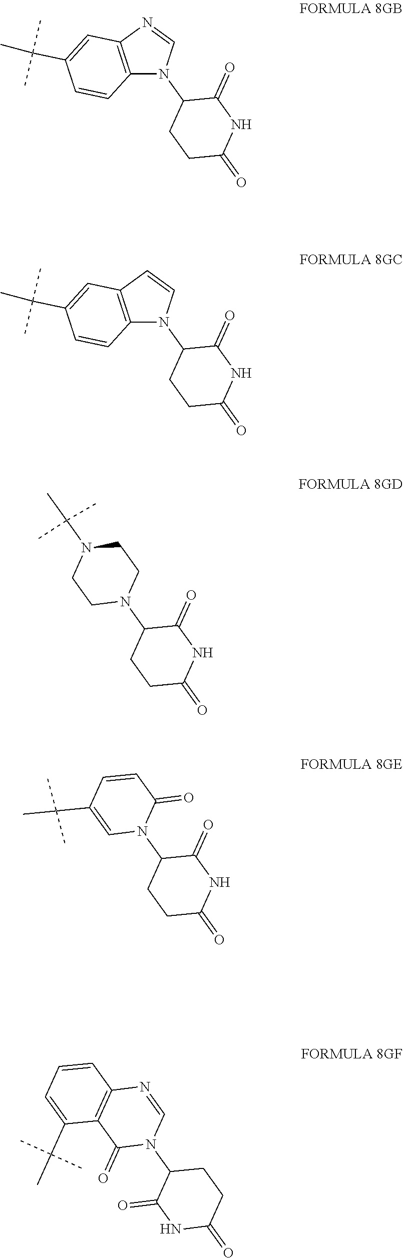

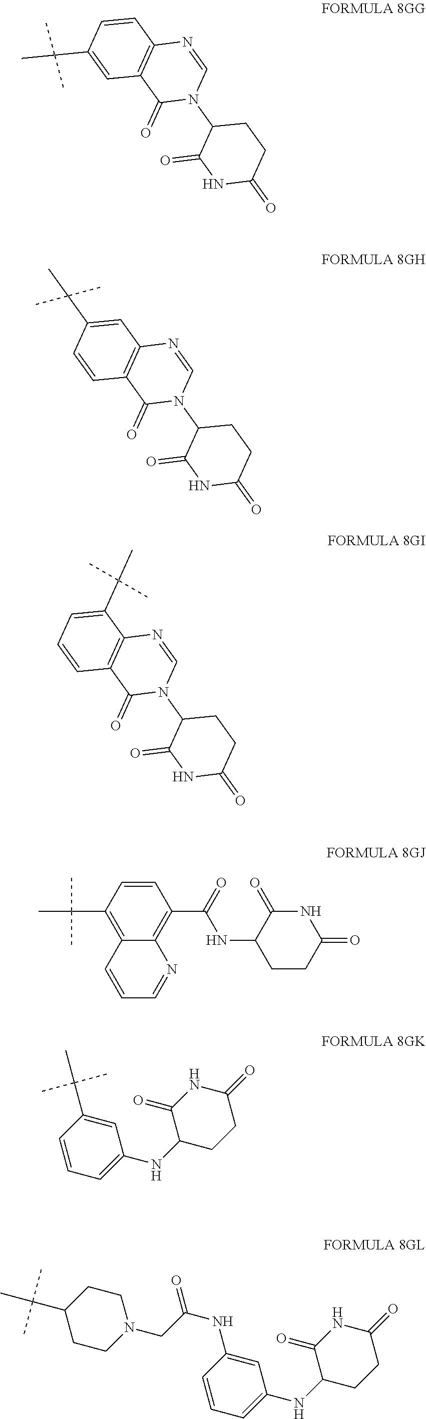

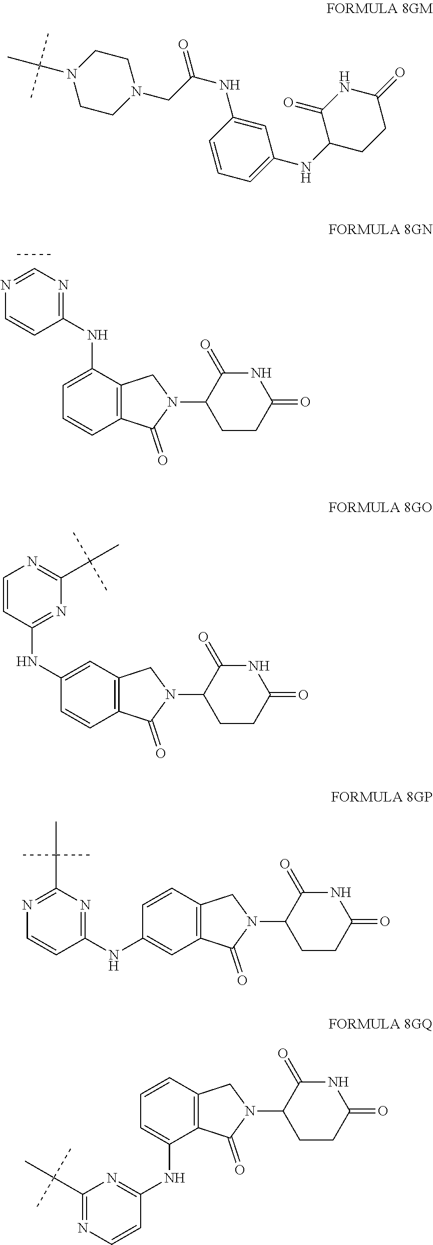

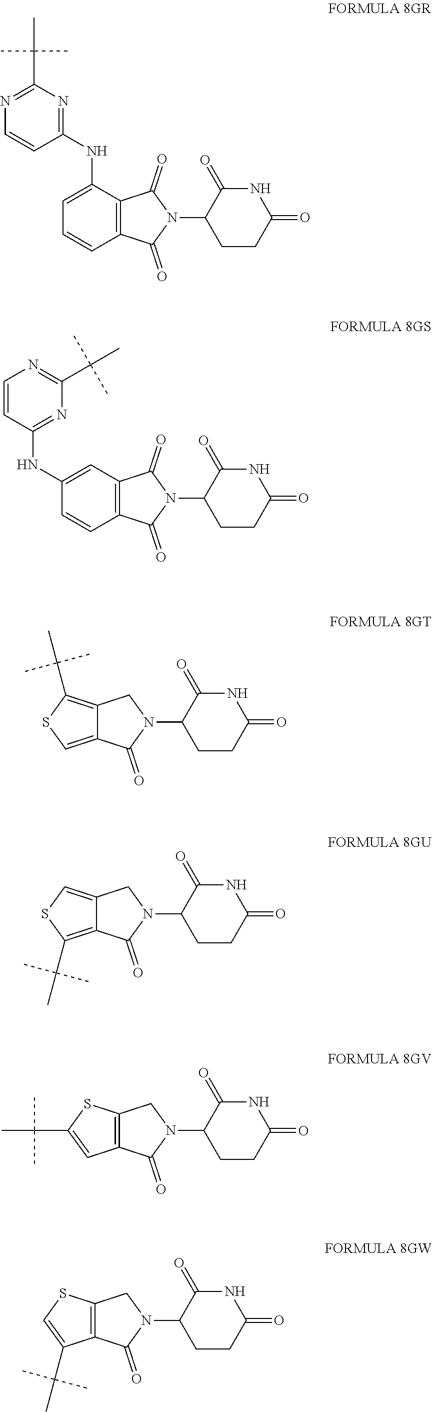

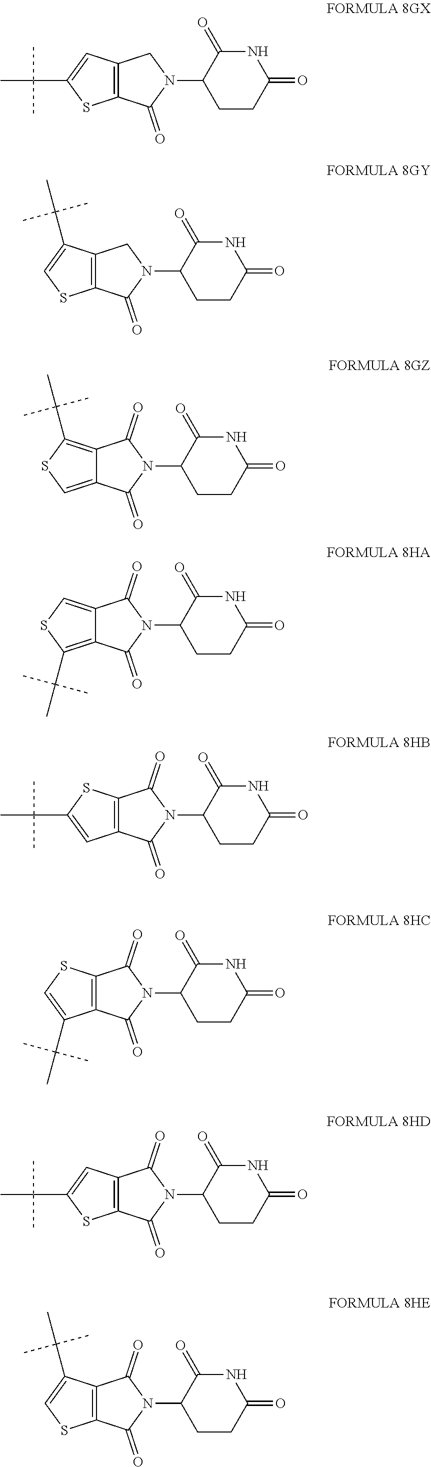

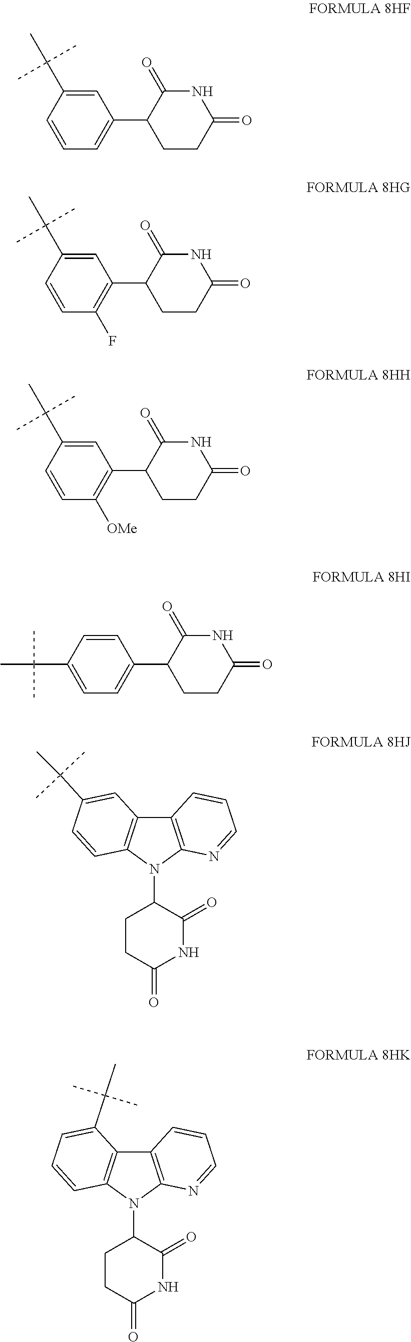

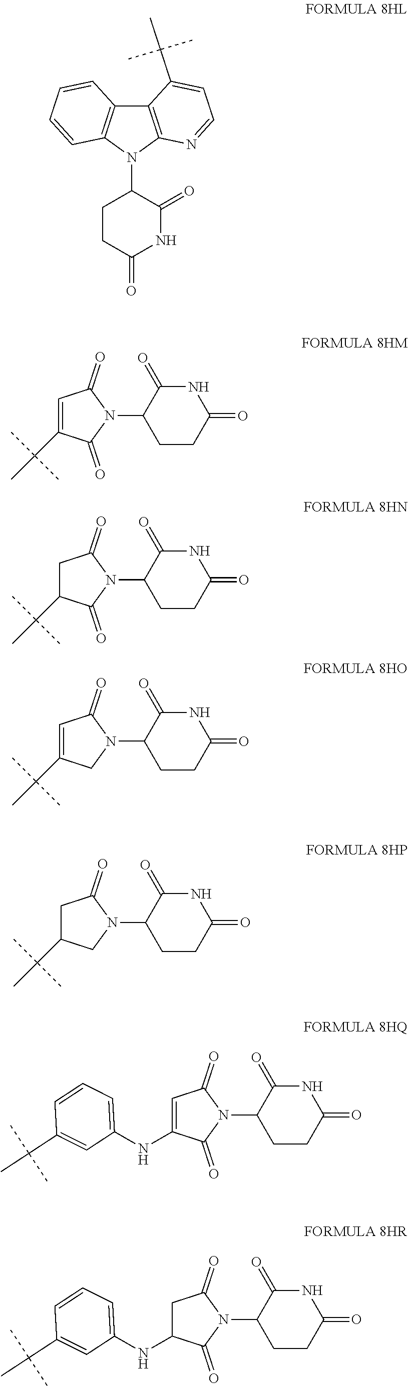

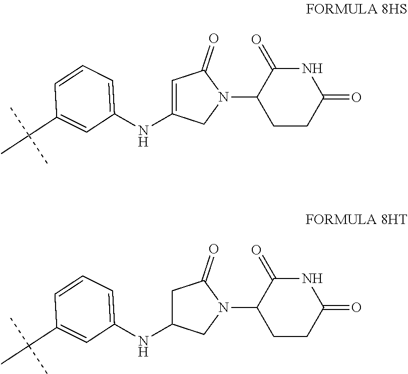

In another embodiment, the degradation tag is a moiety of FORMULA 8A, 8B, 8G or 8H

In another embodiment, the degradation tag is a moiety of FORMULA 8A, 8B, 8C, 8D, 8E, 8F, 8S, 8U, 8W, 8Y, 8AA, 8AC.

In another embodiment, the degradation tag is a moiety of FORMULA 8A or 8G (preferably, 8A).

In another embodiment, the degradation tag is a moiety of FORMULA 4A:

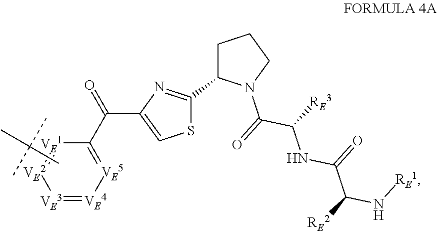

wherein

V_{E}¹, V_{E}², V_{E}³, V_{E}⁴, and V_{E}⁵, are independently selected from CR_{E}⁴ and N; and

R_{E}¹, R_{E}², R_{E}³, and R_{E}⁴ are independently selected from hydrogen, halogen, cyano, nitro, optionally substituted C₁-C₈ alkyl, optionally substituted C₂-C₈ alkenyl, and optionally substituted C₂-C₈ alkynyl; optionally substituted C₁-C₈alkoxyC₁-C₈alkyl, optionally substituted C₁-C₈ haloalkyl, optionally substituted C₁-C₅ hydroxyalkyl, optionally substituted C₁-C₈alkoxy, optionally substituted C₁-C₈alkylamino, optionally substituted C₃-C₁₀ carbocyclyl, and optionally substituted 3-10 membered heterocyclyl.

In another embodiment, the degradation tag is a moiety of FORMULA 4B:

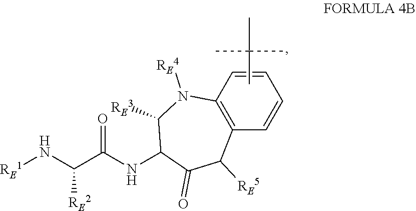

wherein

R_{E}¹, R_{E}², and R_{E}³ are independently selected from hydrogen, halogen, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₃-C₇ cycloalkyl, optionally substituted 3-7 membered heterocyclyl, optionally substituted C₂-C₈ alkenyl, and optionally substituted C₂-C₈ alkynyl;

R_{E}⁴ and R_{E}⁵ are independently selected from hydrogen, COR_{E}⁶, CO₂R_{E}⁶, CONR_{E}⁶R_{E}⁷, SOR_{E}⁶, SO₂R_{E}⁶, SO₂NR_{E}⁶R_{E}⁷, optionally substituted C₁-C₈ alkyl, optionally substituted C₁-C₈alkoxyC₁-C₈alkyl, optionally substituted C₁-C₈alkylaminoC₁-C₈alkyl, optionally substituted aryl-C₁-C₈alkyl, optionally substituted 3-8 membered cycloalkyl, optionally substituted 3-8 membered heterocyclyl, optionally substituted aryl, and optionally substituted heteroaryl, wherein

R_{E}⁶ and R_{E}⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted 3-8 membered cycloalkyl, optionally substituted 3-8 membered heterocyclyl, optionally substituted aryl, and optionally substituted heteroaryl, or R_{E}⁶ and R_{E}⁷ together with the atom(s) to which they are connected form a 3-8 membered cycloalkyl or heterocyclyl ring.

In another embodiment, the degradation tag is a moiety selected from FORMULAE 6A, 6B, and 6C.

In another embodiment, the degradation tag is a moiety of FORMULA 6A.

In another embodiment, the degradation tag is a moiety selected from FORMULAE 6A-1 to 6A-13.

In another embodiment, the degradation tag is a moiety selected from FORMULAE 7A to 7T.

In another embodiment, the degradation tag is a moiety selected from FORMULAE 7A, 7B, 7F, 7G, 7K, 7L, 7P, and 7Q.

In another embodiment, the degradation tag is a moiety of FORMULA 5.

In another embodiment, the degradation tag is a moiety selected from FORMULAE 5-1, 5A, and 5B.

In some embodiments, the linker comprises acyclic or cyclic saturated or unsaturated carbon, ethylene glycol, amide, amino, ether, urea, carbamate, aromatic, heteroaromatic, heterocyclic or carbonyl groups.

In certain embodiments, the length of the linker is 0, 1, 2, 3, 4, 5, 6, 7, 8, 9, 10, 11, 12, 13, 14, 15, 16, 17, 18, 19, 20 or more atoms.

In another embodiment, A_{L} and B_{L}, at each occurrence, are independently selected from the group consisting of null, R_{L}^{d}—R_{L}^{e}, R_{L}^{d}COR_{L}^{e}, R_{L}^{d}C(O)OR_{L}^{e}, R_{L}^{d}C(O)N(R_{L}¹)R_{L}^{e}, R_{L}^{d}OR_{L}^{e}, R_{L}^{d}SR_{L}^{e}, R_{L}^{d}N(R_{L}¹)R_{L}^{e}, R_{L}^{d}N(R_{L}¹)COR_{L}^{e}; wherein R_{L}^{d} and R_{L}^{e}, at each occurrence, are independently selected from the group consisting of null, optionally substituted C₁, C₂ or C₃ alkylene, R_{L}^{r}, R_{L}^{r}-(C₁, C₂ or C₃ alkylene), (C₁, C₂ or C₃ alkylene)-R_{L}^{r}, and (C₁, C₂ or C₃ alkylene)-R_{L}^{r}-(C₁, C₂ or C₃ alkylene).

In another embodiment, A_{L} and B_{L}, at each occurrence, are independently selected from the group consisting of null, R_{L}^{d}—R_{L}^{e}, R_{L}^{d}COR_{L}^{e}, R_{L}^{d}C(O)OR_{L}^{e}, R_{L}^{d}C(O)N(R_{L}¹)R_{L}^{e}, R_{L}^{d}OR_{L}^{e}, R_{L}^{d}SR_{L}^{e}, R_{L}^{d}N(R_{L}¹)R_{L}^{e}, R_{L}^{d}N(R_{L}¹)COR_{L}^{e}; wherein R_{L}^{d} and R_{L}^{e}, at each occurrence, are independently selected from the group consisting of null, R_{L}^{r}, and optionally substituted C₁, C₂ or C₃ alkylene.

In another embodiment, W_{L}¹ and W_{L}², at each occurrence, are independently selected from null, O, S, NH, R_{L}^{r}, optionally substituted C₁-C₃ alkylene, with the proviso that at least one of W_{L}¹ and W_{L}² is not null.

In another embodiment, none of W_{L}¹-W_{L}², A_{L}-W_{L}¹ and W_{L}²-B_{L} is a moiety of —O—O—.

In another embodiment, W_{L}², at each occurrence, is independently null, O, or NH; and W_{L}¹, at each occurrence, is independently selected from R_{L}^{r}, and optionally substituted C₁, C₂ or C₃ alkylene.

In another embodiment, W_{L}¹, at each occurrence, is independently null, O, or NH; and W_{L}², at each occurrence, is independently selected from R_{L}^{r}, and optionally substituted C₁, C₂ or C₃ alkylene.

In another embodiment, A_{L} is the attachment to the TYK2 ligand;

A_{L} is selected from R_{L}^{d}—R_{L}^{e}, R_{L}^{d}C(O)R_{L}^{e}, R_{L}^{d}C(O)NHR_{L}^{e}, R_{L}^{d}NHC(O)R_{L}^{e}, R_{L}^{d}C(O)NHR_{L}^{e}, and R_{L}^{d}NHC(O)R_{L}^{e};

B_{L} is selected from the group consisting of null, R_{L}^{d}C(O)NHR_{L}^{e}, R_{L}^{d}C(O)R_{L}^{e}, R_{L}^{d}NHC(O)R_{L}^{e}, R_{L}^{d}NHR_{L}^{e};

R_{L}^{d} and R_{L}^{e}, at each occurrence, are independently selected from the group consisting of null, optionally substituted C₁, C₂ or C₃ alkylene, R_{L}^{r}, R_{L}^{r}-(C₁, C₂ or C₃ alkylene), (C₁, C₂ or C₃ alkylene)-R_{L}^{r}, and (C₁, C₂ or C₃ alkylene)-R_{L}^{r}-(C₁, C₂ or C₃ alkylene);

W_{L}², at each occurrence, is independently selected from null, O, or NH; and W_{L}¹, at each occurrence, is independently selected from R_{L}r, and optionally substituted C₁, C₂ or C₃ alkylene.

In another embodiment, A_{L} is the attachment to the TYK2 ligand;

A_{L} is selected from the group consisting of R_{L}^{d}—R_{L}^{e}, R_{L}^{d}C(O)R_{L}^{e}, R_{L}^{d}C(O)NHR_{L}^{e}, R_{L}^{d}NHC(O)R_{L}^{e}, R_{L}^{d}C(O)NHR_{L}^{e}, and R_{L}^{d}NHC(O)R_{L}^{e};

B_{L} is selected from the group consisting of null, R_{L}^{d}C(O)NHR_{L}^{e}, R_{L}^{d}C(O)R_{L}^{e}, R_{L}^{d}NHC(O)R_{L}^{e}, and R_{L}^{d}NHR_{L}^{e};

R_{L}^{d} and R_{L}^{e}, at each occurrence, are independently selected from the group consisting of null, optionally substituted C₁, C₂ or C₃ alkylene, R_{L}^{r}, R_{L}^{r}-(C₁, C₂ or C₃ alkylene), (C₁, C₂ or C₃ alkylene)-R_{L}^{r}, and (C₁, C₂ or C₃ alkylene)-R_{L}^{r}-(C₁, C₂ or C₃ alkylene);

W_{L}², at each occurrence, is independently selected from null, O, or NH, and W_{L}¹, at each occurrence, is independently selected from R_{L}^{r}, and optionally substituted C₁, C₂ or C₃ alkylene.

In another embodiment, A_{L} is the attachment to the TYK2 ligand;

A_{L} is selected from the group consisting of R_{L}^{d}—R_{L}^{e}, R_{L}^{d}C(O)R_{L}^{e}, R_{L}^{d}C(O)NHR_{L}^{e}, R_{L}^{d}NHC(O)R_{L}^{e}, R_{L}^{d}C(O)NHR_{L}^{e}, and R^{d}NHC(O)R_{L}^{e};

B_{L} is selected from the group consisting of null, R_{L}^{d}C(O)NHR_{L}^{e}, R_{L}^{d}C(O)R_{L}^{e}, R_{L}^{d}NHC(O)R_{L}^{e}, R_{L}^{d}NHR_{L}^{e};

R_{L}^{d} and R_{L}^{e}, at each occurrence, are independently selected from the group consisting of null, R_{L}^{r}, optionally substituted C₁, C₂ or C₃ alkylene;

W_{L}² is null; and W_{L}¹, at each occurrence, is optionally independently selected from R_{L}^{r}, optionally substituted C₁, C₂ or C₃ alkylene;

m_{L}=4, 5, 6, 7, 8, 9 or 10 (preferably 5, 6,7 or 8).

In another refinement, the length of the linker is 3 to 30 chain atoms.

In another refinement, the length of the linker is 2 to 24 chain atoms.

In another refinement, the length of the linker is 2 to 12 chain atoms.

In another embodiment, R_{L}^{r}, at each occurrence, is selected from FORMULAE C1, C2, C3, C4, and C5

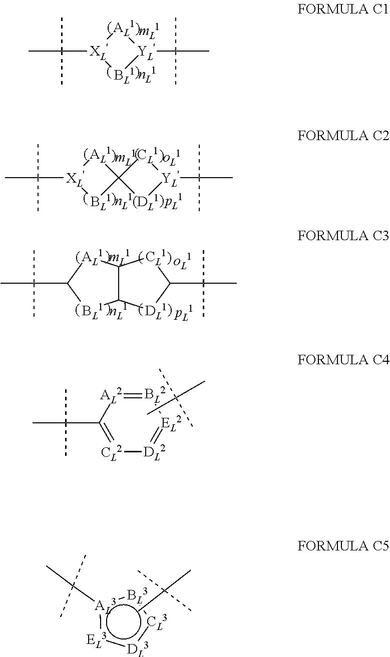

wherein

A_{L}¹, B_{L}¹, C_{L}¹ and D_{L}¹, at each occurrence, are independently selected from the group consisting of null, O, CO, SO, SO₂, NR_{L}^{b}, CR_{L}^{b}R_{L}^{c};

X_{L}′, Y_{L}′, A_{L}², B_{L}², C_{L}², D_{L}² and E_{L}², at each occurrence, are independently selected from N, CR_{L}^{b};

A_{L}³, B_{L}³, C_{L}³, D_{L}³, and E_{L}³, at each occurrence, are independently selected from N, O, S, NR_{L}^{b}, CR_{L}^{b};

R_{L}^{b} and R_{L}^{c}, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted C₃-C₁₀ carbocyclyl, optionally substituted C₃-C₁₀ cycloalkoxy, optionally substituted C₃-C₁₀ carbocyclylamino, optionally substituted 3-10 membered heterocyclyl, optionally substituted aryl, and optionally substituted heteroaryl; and

m_{L}¹, n_{L}¹, o_{L}¹ and p_{L}¹ are independently selected from 0, 1, 2, 3, 4 and 5.

In another embodiment, R_{L}^{r}, at each occurrence, is selected from Group R_{L}^{r1} and Group R_{L}^{r2}, and

Group R_{L}^{r1} consists of optionally substituted following cyclic groups

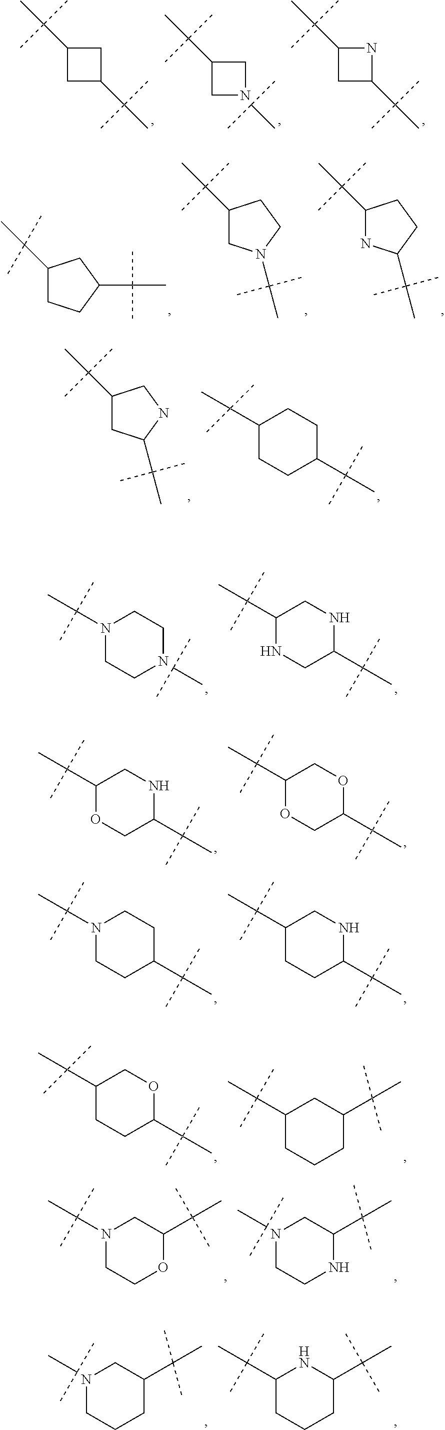

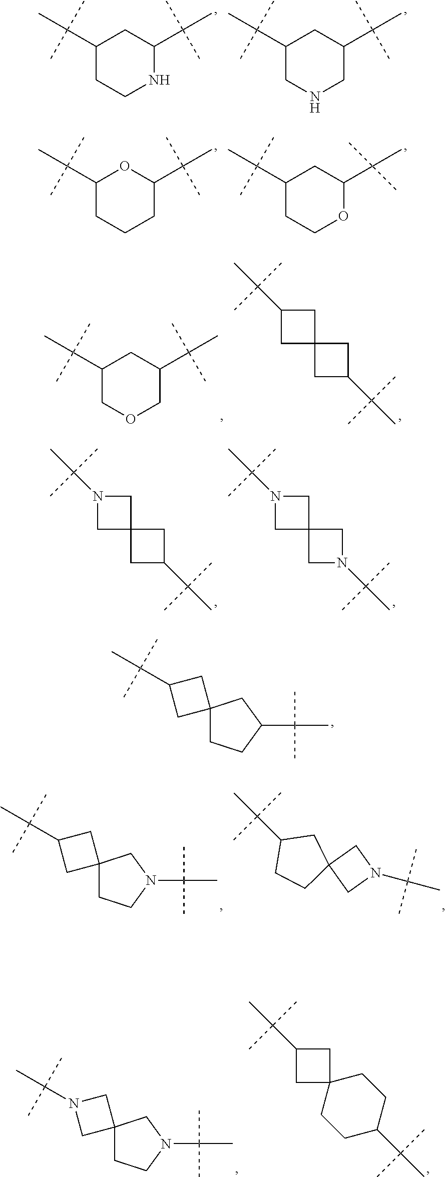

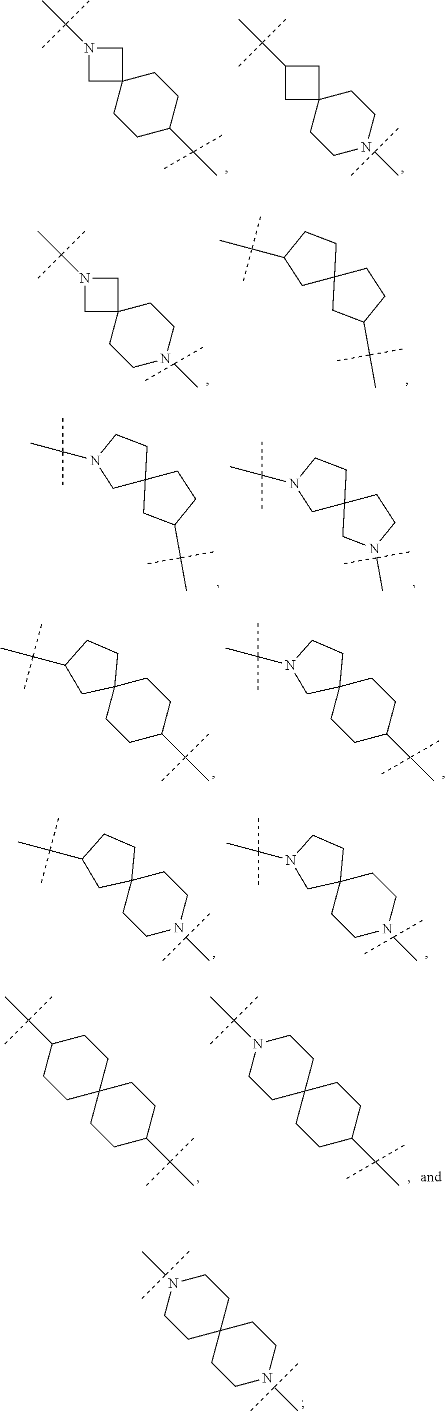

Group R_{L}^{r2} consists of optionally substituted following cyclic groups

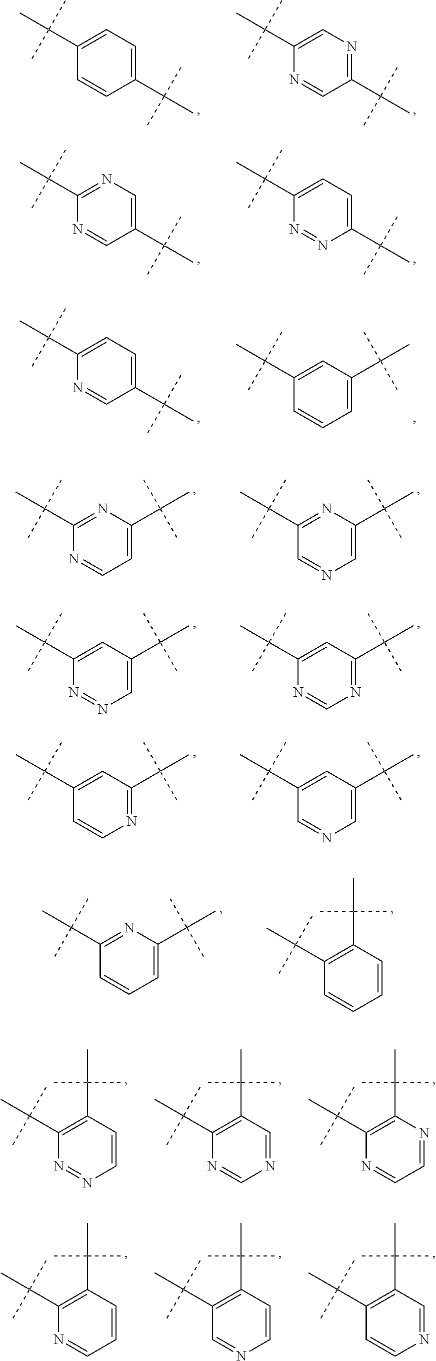

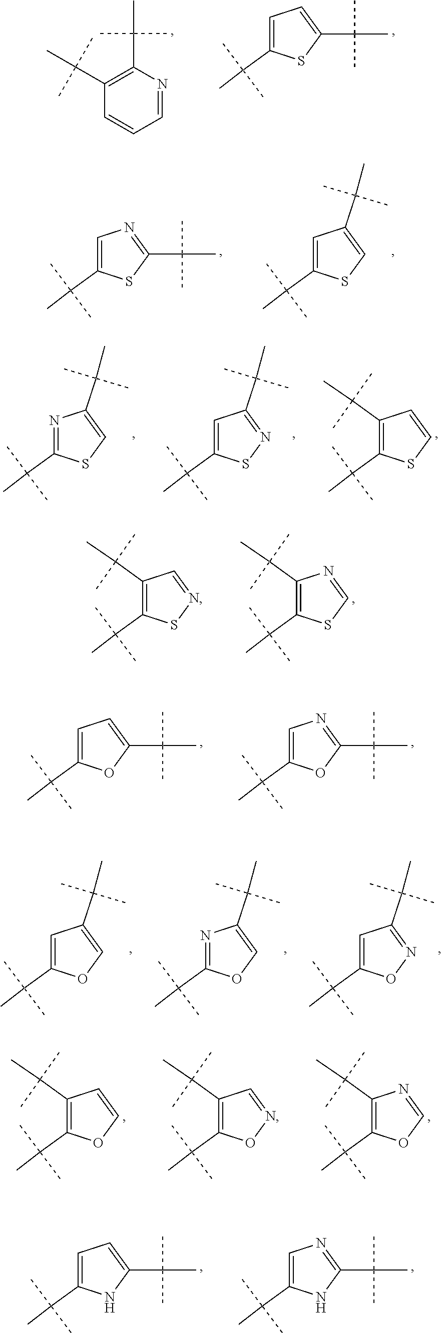

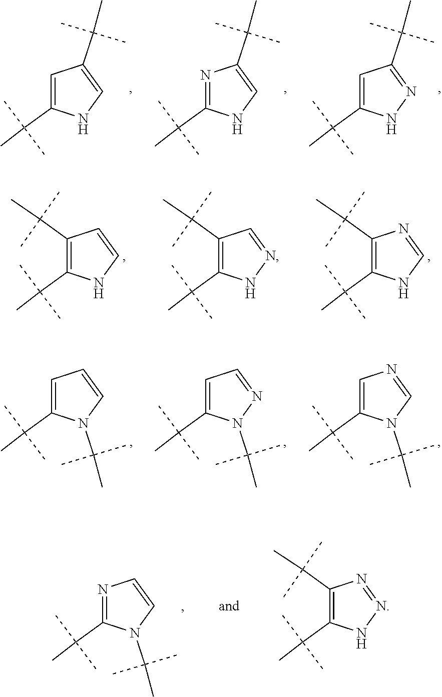

In one embodiment, the linker moiety is of FORMULA 9A:

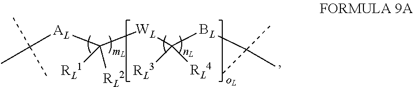

wherein

R_{L}¹, R_{L}², R_{L}³ and R_{L}⁴, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈heteroalkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted C₃-C₁₀ carbocyclyl, optionally substituted C₃-C₁₀ cycloalkoxy, optionally substituted C₃-C₁₀ carbocyclylamino, optionally substituted 3-10 membered heterocyclyl, optionally substituted aryl, and optionally substituted heteroaryl, (preferably, R_{L}¹, R_{L}², R_{L}³ and R_{L}⁴, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted C₃-C₁₀ carbocyclyl, optionally substituted C₃-C₁₀ cycloalkoxy, optionally substituted C₃-C₁₀ carbocyclylamino, optionally substituted 3-10 membered heterocyclyl, optionally substituted aryl, and optionally substituted heteroaryl) or

R_{L}¹ and R_{L}², R_{L}³ and R_{L}⁴ together with the atom(s) to which they are connected form an optionally substituted 3-20 membered cycloalkyl or optionally substituted 3-20 membered heterocyclyl ring;

A_{L}, W_{L} and B_{L}, at each occurrence, are bivalent moieties independently selected from null, R_{L}^{d}—R_{L}^{e}, R_{L}^{d}COR_{L}^{e}, R_{L}^{d}C(O)OR_{L}^{e}, R_{L}^{d}C(O)N(R_{L}⁵)R_{L}^{e}, R_{L}^{d}C(S)N(R_{L}⁵)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}⁵)R_{L}^{e}, R_{L}^{d}N(R_{L}⁵)R_{L}^{e}, R_{L}^{d}N(R_{L}⁵)COR_{L}^{e}, R_{L}^{d}N(R_{L}⁵)CON(R_{L}⁶)R_{L}^{e}, R_{L}^{d}N(R_{L}⁵)C(S)R_{L}^{e}, optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ carbocyclyl, optionally substituted 3-13 membered heterocyclyl, optionally substituted aryl, and optionally substituted heteroaryl, (preferably, A_{L}, W_{L} and B_{L}, at each occurrence, are bivalent moieties independently selected from null, R_{L}^{d}—R_{L}^{e}, R_{L}^{d}COR_{L}^{e}, R_{L}^{d}C(O)OR_{L}^{e}, R_{L}^{d}C(O)N(R_{L}⁵)R_{L}^{e}, R_{L}^{d}C(S)N(R_{L}⁵)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}⁵)R_{L}^{e}, R_{L}^{d}N(R_{L}⁵)R_{L}^{e}, R_{L}^{d}N(R_{L}⁵)COR_{L}^{e}, R_{L}^{d}N(R_{L}⁵)CON(R_{L}⁶)R_{L}^{e}, R_{L}^{d}N(R_{L}⁵)C(S)R_{L}^{e}, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ carbocyclyl, optionally substituted 3-13 membered heterocyclyl, optionally substituted aryl, and optionally substituted heteroaryl) wherein

R_{L}^{d} and R_{L}^{e}, at each occurrence, are independently selected from null, optionally substituted (C₁-C₈ alkyl)-R_{L}^{r} (preferably, CH₂—R_{L}^{r}), optionally substituted R_{L}^{r}-(C₁-C₈ alkylene), optionally substituted (C₁-C₈ alkylene)-R_{L}^{r}-(C₁-C₈ alkylene), or a moiety comprising of optionally substituted C₁-C₈ alkylene, optionally substituted C₂-C₈ alkenylene, optionally substituted C₂-C₈ alkynylene, optionally substituted 1 C₁-C₈ heteroalkylene, optionally substituted C₂-C₈ heteroalkenylene, optionally substituted C₂-C₈ heteroalkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted C₃-C₁₃ carbocyclyl, optionally substituted 3-13 membered heterocyclyl, optionally substituted aryl, and optionally substituted heteroaryl (preferably, R_{L}^{d} and R_{L}^{e}, at each occurrence, are independently selected from null, optionally substituted (C₁-C₈ alkyl)-R_{L}^{r} (preferably, CH₂—R_{L}^{r}), optionally substituted R_{L}^{r}-(C₁-C₈ alkylene), optionally substituted (C₁-C₈ alkylene)-R_{L}^{r}-(C₁-C₈ alkylene), or a moiety comprising of optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted C₃-C₁₃ carbocyclyl, optionally substituted 3-13 membered heterocyclyl, optionally substituted aryl, and optionally substituted heteroaryl);

R_{L}^{r} is defined as in FORMULA 9;

R_{L}⁵ and R_{L}⁶, at each occurrence, are independently selected from hydrogen, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₀ carbocyclyl, optionally substituted 3-10 membered heterocyclyl, optionally substituted aryl, and optionally substituted heteroaryl ((preferably, R_{L}⁵ and R_{L}⁶, at each occurrence, are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₀ carbocyclyl, optionally substituted 3-10 membered heterocyclyl, optionally substituted aryl, and optionally substituted heteroaryl);

R_{L}^{d} and R_{L}^{e}, R_{L}⁵ and R_{L}⁶, R_{L}^{d} and R_{L}⁵, R_{L}^{d} and R_{L}⁶, R_{L}^{e} and R_{L}⁵, R_{L}^{e} and R_{L}⁶ together with the atom(s) to which they are connected optionally form a 3-20 membered cycloalkyl or 3-20 membered heterocyclyl ring;

m_{L} is 0, 1, 2, 3, 4, 5, 6, 7, 8, 9, 10, 11, 12, 13, 14, or 15;

n_{L}, at each occurrence, is 0, 1, 2, 3, 4, 5, 6, 7, 8, 9, 10, 11, 12, 13, 14, or 15; and

o_{L} is 0, 1, 2, 3, 4, 5, 6, 7, 8, 9, 10, 11, 12, 13, 14, or 15.

In another embodiment, the linker moiety is of FORMULA 9B:

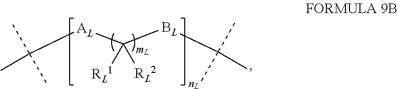

wherein

R_{L}¹ and R_{L}², at each occurrence, are independently selected from hydrogen, halogen, hydroxyl, amino, cyano, nitro, and optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, C₁-C₈alkylaminoC₁-C₈alkyl, optionally substituted C₃-C₁₀ carbocyclyl, optionally substituted C₃-C₁₀ cycloalkoxy, optionally substituted C₃-C₁₀ carbocyclylamino, optionally substituted 3-10 membered heterocyclyl, optionally substituted aryl, and optionally substituted heteroaryl, ((preferably, R_{L}¹ and R_{L}², at each occurrence, are independently selected from hydrogen, halogen, hydroxyl, amino, cyano, nitro, and optionally substituted C₁-C₈ alkyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, C₁-C₈alkylaminoC₁-C₈alkyl, optionally substituted C₃-C₁₀ carbocyclyl, optionally substituted C₃-C₁₀ cycloalkoxy, optionally substituted C₃-C₁₀ carbocyclylamino, optionally substituted 3-10 membered heterocyclyl, optionally substituted aryl, and optionally substituted heteroaryl) or

R_{L}¹ and R_{L}² together with the atom(s) to which they are connected optionally form a 3-20 membered cycloalkyl or 3-20 membered heterocyclyl ring;

A_{L} and B_{L}, at each occurrence, are independently selected from null, or bivalent moiety selected from R_{L}^{d}—R_{L}^{e}, R_{L}^{d}COR_{L}^{e}, R_{L}^{d}CO₂R_{L}^{e}, R_{L}^{d}C(O)N(R_{L}³)R_{L}^{e}, R_{L}^{d}C(S)N(R_{L}³)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}³)R_{L}^{e}, R_{L}^{d}N(R_{L}³)R_{L}^{e}, R_{L}^{d}N(R_{L}³)COR_{L}^{e}, R_{L}^{d}N(R_{L}³)CON(R_{L}⁴)R_{L}^{e}, R_{L}^{d}N(R_{L}³)C(S)R_{L}^{e}, optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ carbocyclyl, optionally substituted 3-13 membered heterocyclyl, optionally substituted aryl, and optionally substituted heteroaryl (preferably, A_{L} and B_{L}, at each occurrence, are independently selected from null, or bivalent moiety selected from R_{L}^{d}—R_{L}^{e}, R_{L}^{d}COR_{L}^{e}, R_{L}^{d}CO₂R_{L}^{e}, R_{L}^{d}C(O)N(R_{L}³)R_{L}^{e}, R_{L}^{d}C(S)N(R_{L}³)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}³)R_{L}^{e}, R_{L}^{d}N(R_{L}³)R_{L}^{e}, R_{L}^{d}N(R_{L}³)COR_{L}^{e}, R_{L}^{d}N(R_{L}³)CON(R_{L}⁴)R_{L}^{e}, R_{L}^{d}N(R_{L}³)C(S)R_{L}^{e}, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ carbocyclyl, optionally substituted 3-13 membered heterocyclyl, optionally substituted aryl, and optionally substituted heteroaryl), wherein

R_{L}^{d} and R_{L}^{e}, at each occurrence, are independently selected from null, optionally substituted (C₁-C₈ alkylene)-R_{L}^{r} (preferably, CH₂—R_{L}^{r}), optionally substituted R_{L}^{r}-(C₁-C₈ alkylene), optionally substituted (C₁-C₈ alkylene)-R_{L}^{r}-(C₁-C₈ alkylene), or a moiety comprising of optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted C₃-C₁₃ carbocyclyl, optionally substituted 3-13 membered heterocyclyl, optionally substituted aryl, and optionally substituted heteroaryl (preferably, R_{L}^{d} and R_{L}^{e}, at each occurrence, are independently selected from null, optionally substituted (C₁-C₈ alkylene)-R_{L}^{r} (preferably, CH₂—R_{L}^{r}), optionally substituted R_{L}^{r}-(C₁-C₈ alkylene), optionally substituted (C₁-C₈ alkylene)-R_{L}^{r}-(C₁-C₈ alkylene), or a moiety comprising of optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted C₃-C₁₃ carbocyclyl, optionally substituted 3-13 membered heterocyclyl, optionally substituted aryl, and optionally substituted heteroaryl);

R_{L}^{r} is defined as in FORMULA 9;

R_{L}³ and R_{L}⁴, at each occurrence, are independently selected from hydrogen, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₀ carbocyclyl, optionally substituted 3-10 membered heterocyclyl, optionally substituted aryl, and optionally substituted heteroaryl (preferably, R_{L}³ and R_{L}⁴, at each occurrence, are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₀ carbocyclyl, optionally substituted 3-10 membered heterocyclyl, optionally substituted aryl, and optionally substituted heteroaryl);

R_{L}^{d} and R_{L}^{e}, R_{L}³ and R_{L}⁴, R_{L}^{d} and R_{L}³, R_{L}^{d} and R_{L}⁴, R_{L}^{e} and R_{L}³, R_{L}^{e} and R_{L}⁴ together with the atom(s) to which they are connected optionally form a 3-20 membered cycloalkyl or 3-20 membered heterocyclyl ring;

each m_{L} is 0, 1, 2, 3, 4, 5, 6, 7, 8, 9, 10, 11, 12, 13, 14, or 15; and

n_{L} is 0, 1, 2, 3, 4, 5, 6, 7, 8, 9, 10, 11, 12, 13, 14, or 15.

In another embodiment, the linker moiety is of FORMULA 9C:

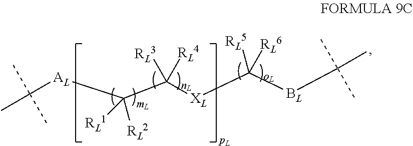

wherein

X_{L}, at each occurrence, is selected from O and NR_{L}⁷;

R_{L}¹, R_{L}², R_{L}³, R_{L}⁴, R_{L}⁵, and R_{L}⁶, at each occurrence, are independently selected from the group consisting of hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, optionally substituted C₁-C₈ alkylaminoC₁-C₈ alkyl, optionally substituted C₃-C₁₀ carbocyclyl, optionally substituted C₃-C₁₀ cycloalkoxy, optionally substituted 3-10 membered heterocyclyl, optionally substituted aryl, and optionally substituted heteroaryl (preferably, R_{L}¹, R_{L}², R_{L}³, R_{L}⁴, R_{L}⁵, and R_{L}⁶, at each occurrence, are independently selected from the group consisting of hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxy C₁-C₅ alkyl, optionally substituted C₁-C₈ haloalkyl, optionally substituted C₁-C₈ hydroxyalkyl, optionally substituted C₁-C₅ alkylamino, optionally substituted C₁-C₅ alkylaminoC₁-C₅ alkyl, optionally substituted C₃-C₁₀ carbocyclyl, optionally substituted C₃-C₁₀ cycloalkoxy, optionally substituted 3-10 membered heterocyclyl, optionally substituted aryl, and optionally substituted heteroaryl);

A_{L} and B_{L}, at each occurrence, are independently selected from null, or bivalent moiety selected from R_{L}^{d}—R_{L}^{e}, R_{L}^{d}COR_{L}^{e}, R_{L}^{d}CO₂R_{L}^{e}, R_{L}^{d}C(O)N(R_{L}⁸)R_{L}^{e}, R_{L}^{d}C(S)N(R_{L}⁸)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}⁸)R_{L}^{e}, R_{L}^{d}N(R_{L}⁸)R_{L}^{e}, R_{L}^{d}N(R_{L}⁸)COR_{L}^{e}, R_{L}^{d}N(R_{L}⁸)CON(R_{L}⁹)R_{L}^{e}, R_{L}^{d}N(R_{L}⁸)C(S)R_{L}^{e}, optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₁-C₈alkoxyC₁-C₈alkylene, optionally substituted C₁-C₈ haloalkylene, optionally substituted C₁-C₈ hydroxyalkylene, optionally substituted 4-13 membered fused cycloalkyl, optionally substituted 5-13 membered fused heterocyclyl, optionally substituted 5-13 membered bridged cycloalkyl, optionally substituted 5-13 membered bridged heterocyclyl, optionally substituted 5-13 membered spiro cycloalkyl, optionally substituted 5-13 membered spiro heterocyclyl, optionally substituted C₃-C₁₀ carbocyclyl, optionally substituted 3-10 membered heterocyclyl, optionally substituted aryl, and optionally substituted heteroaryl (preferably, A_{L} and B_{L}, at each occurrence, are independently selected from null, or bivalent moiety selected from R_{L}d—R_{L}^{e}, R_{L}^{d}COR_{L}^{e}, R_{L}^{d}CO₂R_{L}^{e}, R_{L}^{d}C(O)N(R_{L}⁸)R_{L}^{e}, R_{L}^{d}C(S)N(R_{L}⁸)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}⁸)R_{L}^{e}, R_{L}^{d}N(R_{L}⁸)R_{L}^{e}, R_{L}^{d}N(R_{L}⁸)COR_{L}^{e}, R_{L}^{d}N(R_{L}⁸)CON(R_{L}⁹)R_{L}^{e}, R_{L}^{d}N(R_{L}⁸)C(S)R_{L}^{e}, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted 4-13 membered fused cycloalkyl, optionally substituted 5-13 membered fused heterocyclyl, optionally substituted 5-13 membered bridged cycloalkyl, optionally substituted 5-13 membered bridged heterocyclyl, optionally substituted 5-13 membered spiro cycloalkyl, optionally substituted 5-13 membered spiro heterocyclyl, optionally substituted C₃-C₁₀ carbocyclyl, optionally substituted 3-10 membered heterocyclyl, optionally substituted aryl, and optionally substituted heteroaryl), wherein

R_{L}^{d} and R_{L}^{e}, at each occurrence, are independently selected from null, optionally substituted (C₁-C₈ alkylene)-R_{L}^{r} (preferably, CH₂—R_{L}^{r}), optionally substituted R_{L}^{r}-(C₁-C₈ alkylene), optionally substituted (C₁-C₈ alkylene)-R_{L}^{r}-(C₁-C₈ alkylene), or a moiety comprising of optionally substituted C₁-C₈ alkylene, optionally substituted C₂-C₈ alkenylene, optionally substituted C₁-C₈ heteroalkylene, optionally substituted C₂-C₈ heteroalkenylene, optionally substituted C₂-C₈ heteroalkynylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted C₃-C₁₃ carbocyclyl, optionally substituted 3-13 membered heterocyclyl, optionally substituted aryl, and optionally substituted heteroaryl; preferably, R_{L}^{d} and R_{L}^{e}, at each occurrence, are independently selected from null, optionally substituted (C₁-C₈ alkylene)-R_{L}^{r} (preferably, CH₂—R_{L}^{r}), optionally substituted R_{L}^{r}-(C₁-C₈ alkylene), optionally substituted (C₁-C₈ alkylene)-R_{L}^{r}-(C₁-C₈ alkylene), or a moiety comprising of optionally substituted C₁-C₈ alkylene, optionally substituted C₂-C₈ alkenylene, optionally substituted C₁-C₈ heteroalkylene, optionally substituted C₂-C₈ heteroalkenylene, optionally substituted C₂-C₈ heteroalkynylene, optionally substituted C₂-C₈ alkynylene, optionally substituted C₁-C₈ hydroxyalkylene, optionally substituted C₁-C₈alkoxyC₁-C₈alkylene, optionally substituted C1-C₈alkylaminoC₁-C₈alkylene, optionally substituted C₁-C₈ haloalkylene, optionally substituted C₃-C₁₃ carbocyclyl, optionally substituted 3-13 membered heterocyclyl, optionally substituted aryl, and optionally substituted heteroaryl

R_{L}^{r} is defined as in FORMULA 9;

R_{L}⁷, R_{L}⁸ and R_{L}⁹, at each occurrence, are independently selected from hydrogen, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₀ carbocyclyl, optionally substituted 3-10 membered heterocyclyl, optionally substituted aryl, and optionally substituted heteroaryl; preferably, R_{L}⁷, R_{L}⁸ and R_{L}⁹, at each occurrence, are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₀ carbocyclyl, optionally substituted 3-10 membered heterocyclyl, optionally substituted aryl, and optionally substituted heteroaryl;

R_{L}^{d} and R_{L}^{e}, R_{L}⁸ and R_{L}⁹, R_{L}^{d} and R_{L}⁸, R_{L}^{d} and R_{L}⁹, R_{L}^{e} and R_{L}⁸, R_{L}^{e} and R_{L}⁹ together with the atom(s) to which they are connected optionally form a 3-20 membered cycloalkyl or 3-20 membered heterocyclyl ring;

m_{L}, at each occurrence, is 0, 1, 2, 3, 4, 5, 6, 7, 8, 9, 10, 11, 12, 13, 14, or 15;

n_{L}, at each occurrence, is 0, 1, 2, 3, 4, 5, 6, 7, 8, 9, 10, 11, 12, 13, 14, or 15;

o_{L} is 0, 1, 2, 3, 4, 5, 6, 7, 8, 9, 10, 11, 12, 13, 14, or 15; and

p_{L} is 0, 1, 2, 3, 4, 5, 6, 7, 8, 9, 10, 11, 12, 13, 14, or 15.

In another refinement, the length of the linker is 3 to 30 chain atoms. In another refinement, the length of the linker is 2 to 24 chain atoms. In another refinement, the length of the linker is 2 to 12 chain atoms.

In one embodiment,
1) the TYK2 ligand comprises a moiety of FORMULA 1 or 2 (preferably, a moiety of FORMULA 1; more preferably, a moiety of FORMULA 1-1G; even more preferably, a moiety of FORMULA 1-1I); and/or
2) the degradation tag is a moiety of FORMULAE 6A;
and/or
3) the linker moiety is of FORMULA 9, wherein
A_{L}, and B_{L}, at each occurrence, are bivalent moieties independently selected from the group consisting of null, R_{L}^{d}—R_{L}^{e}, R_{L}^{d}COR_{L}^{e}, R_{L}^{d}C(O)OR_{L}^{e}, R_{L}^{d}C(O)N(R_{L}¹)R_{L}^{e}, R_{L}^{d}C(S)N(R_{L}¹)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}¹)R_{L}^{e}, R_{L}^{d}N(R_{L}¹)R_{L}^{e}, R_{L}^{d}N(R_{L}¹)COR_{L}^{e}, R_{L}^{d}N(R_{L}¹)CON(R_{L}¹)R_{L}^{e}, R_{L}^{d}N(R_{L}¹)C(S)R_{L}^{e}, optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ cycloalkyl, optionally substituted 3-13 membered heterocyclyl, optionally substituted aryl, and optionally substituted heteroaryl; and/or
W_{L}¹ and W_{L}², at each occurrence, are bivalent moieties independently selected from the group consisting of null, optionally substituted C₁-C₈ alkylene; and/or
wherein R_{L}^{d}, R_{L}^{e}, R_{L}^{r}, R_{L}¹ and R_{L}² are defined above; and/or
m_{L} is 1, 2, 3, 4, 5, 6, 7, 8, 9, 10, 11, 12, 13, 14, or 15.

In one embodiment,
1) the TYK2 ligand comprises a moiety of FORMULA 1 (preferably, a moiety of FORMULA 1-1G; more preferably, a moiety of FORMULA 1-1I); and preferably R^{1′} is selected from the group consisting of C(O), optionally substituted C(O)—CH₂, optionally substituted pyridinyl, (optionally substituted pyridinyl)-(C₂ alkynylene), and (optionally substituted pyridinyl)-(optionally substituted piperazinyl); preferably, R^{1′} is selected from the group consisting of C(O), C(O)—CH₂,

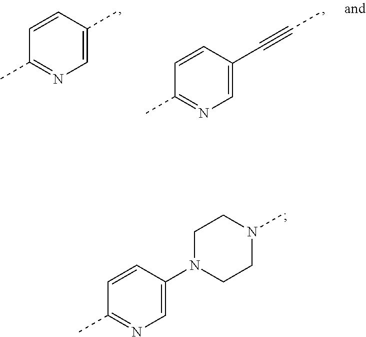

and/or
2) the degradation tag is a moiety of FORMULAE 6A (preferably, a moiety of FORMULA 6A-9 or 6A-10, more preferably, a moiety of FORMULA 7F or 7G);
and/or
3) the linker moiety is of FORMULA 9, wherein A_{L} is the attachment to the TYK2 ligand; and wherein
A_{L} is selected from the group consisting of null, R_{L}^{d}COR_{L}^{e}, R_{L}^{d}C(O)N(R_{L}¹)R_{L}^{e}, R_{L}^{d}N(R_{L}¹)COR_{L}^{e}; preferably, A_{L} is selected from the group consisting of R_{L}^{r}, R_{L}^{r}CO, CO, R_{L}^{r}NHCO, NHCO, R_{L}^{r}CH₂NHCO and CH₂NHCO, R_{L}^{r}CH₂CONH, and CH₂CONH; more preferably, A_{L} is selected from the group consisting of CO, NHCO, CONH, CH₂CONH, CH₂NHCO,

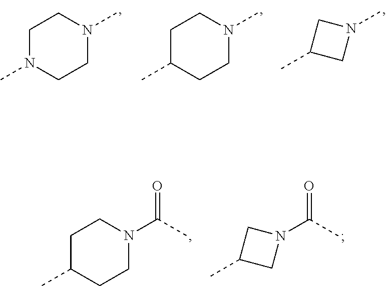

and/or
B_{L} is null or R_{L}^{d}C(O)R_{L}^{e}; preferably, B_{L} is null or C(O); more preferably B_{L} is C(O); and/or
W_{L}² is null, and W_{L}¹ is independently optionally substituted C₁ alkylene; preferably, W_{L}² is null, and W_{L}¹ is C₁ alkylene; and/or
m_{L} is 1, 2, 3, 4, 5, 6, 7, 8, 9, 10, 11, 12, 13, 14, or 15; preferably, m_{L} is 1, 2, 3, 4, 5, 6, 7, or 8.

In one embodiment,
1) the TYK2 ligand comprises a moiety of FORMULA 1 or 2 (preferably, a moiety of FORMULA 1; more preferably, a moiety of FORMULA 1-1G; even more preferably, a moiety of FORMULA 1-1I);
and/or
2) the degradation tag is a moiety of FORMULAE 6B or 6C (preferably, a moiety of FORMULA 6B- or 6C-10, more preferably, a moiety of FORMULA 7AC or 7AQ);
and/or
3) the linker moiety is of FORMULA 9, wherein
A_{L}, and B_{L}, at each occurrence, are bivalent moieties independently selected from the group consisting of null, R_{L}^{d}—R_{L}^{e}, R_{L}^{d}COR_{L}^{e}, R_{L}^{d}C(O)OR_{L}^{e}, R_{L}^{d}C(O)N(R_{L}¹)R_{L}^{e}, R_{L}^{d}C(S)N(R_{L}¹)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}¹)R_{L}^{e}, R_{L}^{d}N(R_{L}¹)R_{L}^{e}, R_{L}^{d}N(R_{L}¹)COR_{L}^{e}, R_{L}^{d}N(R_{L}¹)CON(R_{L}²)R_{L}^{e}, R_{L}^{d}N(R_{L}¹)C(S)R_{L}^{e}, optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₁-C₈alkoxyC₁-C₈alkylene, optionally substituted C₁-C₁ haloalkylene, optionally substituted C₁-C₈ hydroxyalkylene, optionally substituted C₃-C₁₃ cycloalkyl, optionally substituted 3-13 membered heterocyclyl, optionally substituted aryl, and optionally substituted heteroaryl (preferably, A_{L}, and B_{L}, at each occurrence, are bivalent moieties independently selected from the group consisting of null, R_{L}^{d}—R_{L}^{e}, R_{L}^{d}COR_{L}^{e}, R_{L}^{d}C(O)OR_{L}^{e}, R_{L}^{d}C(O)N(R_{L}¹)R_{L}^{e}, R_{L}^{d}C(S)N(R_{L}¹)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}¹)R_{L}^{e}, R_{L}^{d}N(R_{L}¹)R_{L}^{e}, R_{L}^{d}N(R_{L}¹)COR_{L}^{e}, R_{L}^{d}N(R_{L}¹)CON(R_{L}²)R_{L}^{e}, R_{L}^{d}N(R_{L}¹)C(S)R_{L}^{e}, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ cycloalkyl, optionally substituted 3-13 membered heterocyclyl, optionally substituted aryl, and optionally substituted heteroaryl); and/or
W_{L}² is null, and W_{L}¹ is independently optionally substituted C₁, C₂ or C₃ alkylene; preferably, W_{L}² is null, and W_{L}¹ is C₁, C₂ or C₃ alkylene; and/or
wherein R_{L}^{d}, R_{L}^{e}, R_{L}^{r}, R_{L}¹ and R_{L}² are defined above; and/or
m_{L} is 1, 2, 3, 4, 5, 6, 7, 8, 9, 10, 11, 12, 13, 14, or 15 (preferably, m_{L} is 8, 9, 10, 11, 12, 13, 14, or 15).

In one embodiment,
1) the TYK2 ligand comprises a moiety of FORMULA 1 or 2 (preferably, a moiety of FORMULA 1; more preferably, a moiety of FORMULA 1-1G; even more preferably, a moiety of FORMULA 1-1I);
and/or
2) the degradation tag is a moiety of FORMULAE 6B or 6C (preferably, a moiety of FORMULA 6B- or 6C-10, more preferably, a moiety of FORMULA 7AC or 7AQ); and preferably R^{1′} is selected from the group consisting of C(O);
and/or
3) the linker moiety is of FORMULA 9, wherein A_{L} is the attachment to the TYK2 ligand; and wherein
A_{L} is selected from the group consisting of null, R_{L}^{d}COR_{L}^{e}, R_{L}^{d}C(O)N(R_{L}¹)R_{L}^{e}, R_{L}^{d}N(R_{L}¹)COR_{L}^{e}; preferably, A_{L} is selected from the group consisting of NHCO, CONH, CH₂CONH, CH₂NHCO; and/or
B_{L} is null or R_{L}^{d}C(O)R_{L}^{e}; preferably, B_{L} is null or C(O); more preferably B_{L} is C(O); and/or
W_{L}² is null, and W_{L}¹ is independently optionally substituted C₁ alkylene; preferably, W_{L}² is null, and W_{L}¹ is C₁ alkylene; and/or
m_{L} is 1, 2, 3, 4, 5, 6, 7, 8, 9, 10, 11, 12, 13, 14, or 15 (preferably, m_{L} is 8, 9, 10, 11, 12, 13, 14, or 15; more preferably, m_{L} is 8, 9, 10, 11, 12, or 13).

In one embodiment,
1) the TYK2 ligand comprises a moiety of FORMULA 1 or 2 (preferably, a moiety of FORMULA 1; more preferably, a moiety of FORMULA 1-1G; even more preferably, a moiety of FORMULA 1-1I);
and/or
2) the degradation tag is a moiety of FORMULA 5-1 (preferably, a moiety of FORMULAE 5A, more preferably, a moiety of FORMULA 5A and Z_{E} is connected to V_{E}¹ or V_{E}⁴);
and/or
3) the linker moiety is of FORMULA 9, wherein
A_{L}, and B_{L}, at each occurrence, are bivalent moieties independently selected from the group consisting of null, R_{L}^{d}—R_{L}^{e}, R_{L}^{d}COR_{L}^{e}, R_{L}^{d}C(O)OR_{L}^{e}, R_{L}^{d}C(O)N(R_{L}¹)R_{L}^{e}, R_{L}^{d}C(S)N(R_{L}¹)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}¹)R_{L}^{e}, R_{L}^{d}N(R_{L}¹)R_{L}^{e}, R_{L}^{d}N(R_{L}¹)COR_{L}^{e}, R_{L}^{d}N(R_{L}¹)CON(R_{L}²)R_{L}^{e}, R_{L}^{d}N(R_{L}¹)C(S)R_{L}^{e}, optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ cycloalkyl, optionally substituted 3-13 membered heterocyclyl, optionally substituted aryl, and optionally substituted heteroaryl; and/or

W_{L}¹ is independently optionally substituted C₁, C₂ or C₃ alkylene and W_{L}² is null or O; wherein R_{L}^{d}, R_{L}^{e}, R_{L}^{r}, R_{L}¹ and R_{L}² are defined above; and/or

m_{L} is 1, 2, 3, 4, 5, 6, 7, 8, 9, 10, 11, 12, 13, 14, or 15.

In one embodiment,
1) the TYK2 ligand comprises a moiety of FORMULA 1 or 2 (preferably, a moiety of FORMULA 1; more preferably, a moiety of FORMULA 1-1G; even more preferably, a moiety of FORMULA 1-1I);
and/or
2) the degradation tag is a moiety of FORMULA 5A (preferably, a moiety of FORMULA 5A, and Z_{E} is connected to V_{E}¹ or V_{E}⁴; more preferably, a moiety of 8A or 8G) and preferably, R^{1′} is optionally substituted aryl or optionally substituted heteroaryl; preferably optionally substituted pyridinyl;
and/or
3) the linker moiety is of FORMULA 9, wherein A_{L} is the attachment to the TYK2 ligand; and wherein
A_{L} is selected from the group consisting of null, R_{L}^{d}N(R_{L}¹)COR_{L}^{e}, R_{L}^{d}CON(R_{L}¹)R_{L}^{e}; preferably, A_{L} is selected from the group consisting of NHCO, and CONH; and/or
B_{L} is null; and/or
W_{L}¹ is independently optionally substituted C₁, C₂ or C₃ alkylene and W_{L}² is null or O; wherein R_{L}^{d}, R_{L}^{e}, R_{L}^{r}, R_{L}¹ and R_{L}² are defined above; and/or
m_{L} is 1, 2, 3, 4, 5, 6, 7, 8, 9, 10, 11, 12, 13, 14, or 15 (preferably, 4, 5, 6, 7, 8, 9, 10, 11, or 12).

In one embodiment,
1) the TYK2 ligand comprises a moiety of FORMULA 1 or 2 (preferably, a moiety of FORMULA 1; more preferably, a moiety of FORMULA 1-1G; even more preferably, a moiety of FORMULA 1-1I);
and/or
2) the degradation tag is a moiety of FORMULA 5A, and Z_{E} is connected to V_{E}¹ or V_{E}⁴; preferably, is a moiety of 8A) and preferably R^{1′} is selected from the group consisting of C(O), optionally substituted C(O)—CH₂ preferably, R^{1′} is selected from the group consisting of C(O), C(O)—CH₂;
and/or
3) the linker moiety is of FORMULA 9, wherein A_{L} is the attachment to the TYK2 ligand; and wherein
A_{L} is selected from the group consisting of null, R_{L}^{d}N(R_{L}¹)COR_{L}^{e}, R_{L}^{d}CON(R_{L}¹)R_{L}^{e}; preferably, A_{L} is selected from the group consisting of CH₂NHCO, and CH₂CONH, NHCO, and CONH; and/or
B_{L} is null; and/or
W_{L}¹ is independently optionally substituted C₁ alkylene and W_{L}² is null; and/or
m_{L} is 1, 2, 3, 4, 5, 6, 7, 8, 9, 10, 11, 12, 13, 14, or 15 (preferably, 1, 2, 3, 4, 5, 6, 7, 8, 9, or 10).

In some embodiments, the compound comprises any one of the compounds in Table 1, Table 2, or Table 3.

In some embodiments, the heterobifunctional compound is selected from the group consisting of CPD-001 to CPD-199 or a pharmaceutically acceptable salt or analog thereof. In some embodiments, the heterobifunctional compound is selected from the group consisting of CPD-038, CPD-039, CPD-040, CPD-047, CPD-084, CPD-085, CPD-099, CPD-100, CPD-110, CPD-112, CPD-114, CPD-115, CPD-121, CPD-124, CPD-125, CPD-126, CPD-127, CPD-131, CPD-133, CPD-134, CPD-143, CPD-144, CPD-148, CPD-150, CPD-151, CPD-155, CPD-157, CPD-158, CPD-159, CPD-164, CPD-167, CPD-175, and a pharmaceutically acceptable salt or analog thereof.

In some embodiments, the compound comprises CPD-038, CPD-039, CPD-040, CPD-047, CPD-084, CPD-085, CPD-099, CPD-100, CPD-110, CPD-112, CPD-114, CPD-115, CPD-121, CPD-124, CPD-125, CPD-126, CPD-127, CPD-131, CPD-133, CPD-134, CPD-143, CPD-144, CPD-148, CPD-150, CPD-151, CPD-155, CPD-157, CPD-158, CPD-159, CPD-164, CPD-167, CPD-175, or a pharmaceutically acceptable salt or analog thereof.

In one embodiment, the heterobifunctional compound is N1-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N8-(2-((5-((2-methoxy-3-(1-methyl-1H-1,2,4-triazol-3-yl)phenyl)amino)-6-(methylcarbamoyl)pyridazin-3-yl)amino)-2-oxoethyl)octanediamide (CPD-038).

In one embodiment, the heterobifunctional compound is N1-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N9-(2-((5-((2-methoxy-3-(1-methyl-1H-1,2,4-triazol-3-yl)phenyl)amino)-6-(methylcarbamoyl)pyridazin-3-yl)amino)-2-oxoethyl)nonanediamide (CPD-039).

In one embodiment, the heterobifunctional compound is N1-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N10-(2-((5-((2-methoxy-3-(1-methyl-1H-1,2,4-triazol-3-yl)phenyl)amino)-6-(methylcarbamoyl)pyridazin-3-yl)amino)-2-oxoethyl)decanediamide (CPD-040).

In one embodiment, the heterobifunctional compound is 6-(2-(7-((2-(2,6-dioxopiperidin-3-yl)-1,3-dioxoisoindolin-4-yl)amino)heptanamido)acetamido)-4-((2-methoxy-3-(1-methyl-H-1,2,4-triazol-3-yl)phenyl)amino)-N-methylpyridazine-3-carboxamide (CPD-047).

In one embodiment, the heterobifunctional compound is N1-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-N9-(2-((5-((2-methoxy-3-(1-methyl-1H-1,2,4-triazol-3-yl)phenyl)amino)-6-(methylcarbamoyl)pyridazin-3-yl)amino)-2-oxoethyl)nonanediamide (CPD-084).

In one embodiment, the heterobifunctional compound is N1-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-N10-(2-((5-((2-methoxy-3-(1-methyl-1H-1,2,4-triazol-3-yl)phenyl)amino)-6-(methylcarbamoyl)pyridazin-3-yl)amino)-2-oxoethyl)decanediamide (CPD-085).

In one embodiment, the heterobifunctional compound is 6-((5-(4-(7-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-7-oxoheptanoyl)piperazin-1-yl)pyridin-2-yl)amino)-4-((2-methoxy-3-(1-methyl-JH-1,2,4-triazol-3-yl)phenyl)amino)-N-methylpyridazine-3-carboxamide (CPD-099).

In one embodiment, the heterobifunctional compound is 6-((5-(4-(8-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-8-oxooctanoyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-100).

In one embodiment, the heterobifunctional compound is 6-(2-(11-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)undecanamido)acetamido)-4-((2-methoxy-3-(1-methyl-1H-1,2,4-triazol-3-yl)phenyl)amino)-N-methylpyridazine-3-carboxamide (CPD-110).

In one embodiment, the heterobifunctional compound is 6-((5-(4-(8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctanoyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-112).

In one embodiment, the heterobifunctional compound is 6-((5-(4-(10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-114).

In one embodiment, the heterobifunctional compound is 6-((5-(4-(9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononanoyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-115).

In one embodiment, the heterobifunctional compound is 6-((5-((1-(10-(((S)-1-((2S,4R)-4-hydroxy-2-(((5)-1-(4-(4-methylthiazol-5-yl)phenyl)ethyl)carbamoyl)pyrrolidin-1-yl)-3,3-dimethyl-1-oxobutan-2-yl)amino)-10-oxodecanoyl)piperidin-4-yl)ethynyl)pyridin-2-yl)amino)-4-((2-methoxy-3-(1-methyl-1H-1,2,4-triazol-3-yl)phenyl)amino)-N-methylpyridazine-3-carboxamide (CPD-121).

In one embodiment, the heterobifunctional compound is 6-((5-((1-(7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoyl)azetidin-3-yl)ethynyl)pyridin-2-yl)amino)-4-((2-methoxy-3-(1-methyl-1H-1,2,4-triazol-3-yl)phenyl)amino)-N-methylpyridazine-3-carboxamide (CPD-124).

In one embodiment, the heterobifunctional compound is 6-((5-((1-(8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctanoyl)azetidin-3-yl)ethynyl)pyridin-2-yl)amino)-4-((2-methoxy-3-(1-methyl-H-1,2,4-triazol-3-yl)phenyl)amino)-N-methylpyridazine-3-carboxamide (CPD-125).

In one embodiment, the heterobifunctional compound is 6-((5-((1-(9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononanoyl)azetidin-3-yl)ethynyl)pyridin-2-yl)amino)-4-((2-methoxy-3-(1-methyl-H-1,2,4-triazol-3-yl)phenyl)amino)-N-methylpyridazine-3-carboxamide (CPD-126).

In one embodiment, the heterobifunctional compound is 6-((5-((1-(10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoyl)azetidin-3-yl)ethynyl)pyridin-2-yl)amino)-4-((2-methoxy-3-(1-methyl-1H-1,2,4-triazol-3-yl)phenyl)amino)-N-methylpyridazine-3-carboxamide (CPD-127).

In one embodiment, the heterobifunctional compound is N1-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-N8-((6-((5-((2-methoxy-3-(1-methyl-1H-1,2,4-triazol-3-yl)phenyl)amino)-6-(methylcarbamoyl)pyridazin-3-yl)amino)pyridin-3-yl)methyl)octanediamide (CPD-131).

In one embodiment, the heterobifunctional compound is N1-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-N10-((6-((5-((2-methoxy-3-(1-methyl-1H-1,2,4-triazol-3-yl)phenyl)amino)-6-(methylcarbamoyl)pyridazin-3-yl)amino)pyridin-3-yl)methyl)decanediamide (CPD-133).

In one embodiment, the heterobifunctional compound is 6-((5-((1-(5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentanoyl)piperidin-4-yl)ethynyl)pyridin-2-yl)amino)-4-((2-methoxy-3-(1-methyl-1H-1,2,4-triazol-3-yl)phenyl)amino)-N-methylpyridazine-3-carboxamide (CPD-134).

In one embodiment, the heterobifunctional compound is 6-((5-((8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-143).

In one embodiment, the heterobifunctional compound is 6-((5-((9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-144).

In one embodiment, the heterobifunctional compound is 6-((5-(4-(1-(7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoyl)piperidin-4-yl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-148).

In one embodiment, the heterobifunctional compound is 6-((5-(4-(1-(9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononanoyl)piperidin-4-yl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-150).

In one embodiment, the heterobifunctional compound is 6-((5-(4-(1-(10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoyl)piperidin-4-yl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-151).

In one embodiment, the heterobifunctional compound is 6-((5-((5-((2-(2,6-dioxopiperidin-3-yl)-1,3-dioxoisoindolin-4-yl)amino)pentyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-155).

In one embodiment, the heterobifunctional compound is 6-((5-((7-((2-(2,6-dioxopiperidin-3-yl)-1,3-dioxoisoindolin-4-yl)amino)heptyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-157).

In one embodiment, the heterobifunctional compound is 6-((5-((8-((2-(2,6-dioxopiperidin-3-yl)-1,3-dioxoisoindolin-4-yl)amino)octyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-158).

In one embodiment, the heterobifunctional compound is 6-((5-((2-(2-((2-(2,6-dioxopiperidin-3-yl)-1,3-dioxoisoindolin-4-yl)amino)ethoxy)ethyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-159).

In one embodiment, the heterobifunctional compound is 6-((5-((5-((2-(2,6-dioxopiperidin-3-yl)-1,3-dioxoisoindolin-5-yl)amino)pentyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-164).

In one embodiment, the heterobifunctional compound is 6-((5-((8-((2-(2,6-dioxopiperidin-3-yl)-1,3-dioxoisoindolin-5-yl)amino)octyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-167).

In one embodiment, the heterobifunctional compound is 6-((5-((3-((2-(2,6-dioxopiperidin-3-yl)-1,3-dioxoisoindolin-5-yl)amino)propyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-175).

Without wishing to be bound by any particular theory, it is contemplated herein that, in some embodiments, attaching VHL-1 or pomalidomide to either portion of the molecule can recruit the VHL E3 ligase or cereblon E3 ligase to TYK2.

The heterobifunctional compounds disclosed herein can selectively affect TYK2-mediated disease cells compared to WT (wild type) cells (i.e., an heterobifunctional compound able to kill or inhibit the growth of an TYK2-mediated disease cell while also having a relatively low ability to lyse or inhibit the growth of a WT cell), e.g., possess a GI₅₀ for one or more TYK2-mediated disease cells more than 1.5-fold lower, more than 2-fold lower, more than 2.5-fold lower, more than 3-fold lower, more than 4-fold lower, more than 5-fold lower, more than 6-fold lower, more than 7-fold lower, more than 8-fold lower, more than 9-fold lower, more than 10-fold lower, more than 15-fold lower, or more than 20-fold lower than its GI₅₀ for one or more WT cells, e.g., WT cells of the same species and tissue type as the TYK2-mediated disease cells.

In some aspects, provided herein is a method for identifying a heterobifunctional compound which mediates degradation or reduction of TYK2, the method comprising: providing a heterobifunctional test compound comprising an TYK2 ligand conjugated to a degradation tag through a linker; contacting the heterobifunctional test compound with a cell comprising a ubiquitin ligase and TYK2; determining whether TYK2 level is decreased in the cell; and identifying the heterobifunctional test compound as a heterobifunctional compound which mediates degradation or reduction of TYK2. In certain embodiments, the cell is a cancer cell. In certain embodiments, the cancer cell is a TYK2-mediated cancer cell.

Synthesis and Testing of Heterobifunctional Compounds

The binding affinity of novel synthesized heterobifunctional compounds can be assessed using standard biophysical assays known in the art (e.g., isothermal titration calorimetry (ITC), surface plasmon resonance (SPR)). Cellular assays can then be used to assess the heterobifunctional compound's ability to induce TYK2 degradation and inhibit cancer cell proliferation. Besides evaluating a heterobifunctional compound's induced changes in the protein levels of TYK2, TYK2 mutants, TYK2 deletions, or TYK2 fusion proteins, protein-protein interaction or kinase enzymatic activity can also be assessed. Assays suitable for use in any or all of these steps are known in the art, and include, e.g., western blotting, quantitative mass spectrometry (MS) analysis, flow cytometry, enzymatic activity assay, ITC, SPR, cell growth inhibition, xenograft, orthotopic, and patient-derived xenograft models. Suitable cell lines for use in any or all of these steps are known in the art and include MOLT-4, NOMO-1 and PBMC cells. Suitable mouse models for use in any or all of these steps are known in the art and include subcutaneous xenograft models, orthotopic models, patient-derived xenograft models, and patient-derived orthotopic models.

By way of non-limiting example, detailed synthesis protocols are described in the Examples for specific exemplary heterobifunctional compounds.

Pharmaceutically acceptable isotopic variations of the compounds disclosed herein are contemplated and can be synthesized using conventional methods known in the art or methods corresponding to those described in the Examples (substituting appropriate reagents with appropriate isotopic variations of those reagents). Specifically, an isotopic variation is a compound in which at least one atom is replaced by an atom having the same atomic number, but an atomic mass different from the atomic mass usually found in nature. Useful isotopes are known in the art and include, for example, isotopes of hydrogen, carbon, nitrogen, oxygen, phosphorus, sulfur, fluorine, and chlorine. Exemplary isotopes thus include, e.g., ²H, ³H, ¹³C, ¹⁴C, ¹⁵N, ¹⁷O, ¹⁸O, ³²P, ³⁵S, ¹⁸F, and ³⁶Cl.

Isotopic variations (e.g., isotopic variations containing ²H) can provide therapeutic advantages resulting from greater metabolic stability, e.g., increased in vivo half-life or reduced dosage requirements. In addition, certain isotopic variations (particularly those containing a radioactive isotope) can be used in drug or substrate tissue distribution studies. The radioactive isotopes tritium (³H) and carbon-14 (¹⁴C) are particularly useful for this purpose in view of their ease of incorporation and ready means of detection.

Pharmaceutically acceptable solvates of the compounds disclosed herein are contemplated. A solvate can be generated, e.g., by substituting a solvent used to crystallize a compound disclosed herein with an isotopic variation (e.g., D₂O in place of H₂O, d₆-acetone in place of acetone, or d₆-DMSO in place of DMSO).

Pharmaceutically acceptable fluorinated variations of the compounds disclosed herein are contemplated and can be synthesized using conventional methods known in the art or methods corresponding to those described in the Examples (substituting appropriate reagents with appropriate fluorinated variations of those reagents). Specifically, a fluorinated variation is a compound in which at least one hydrogen atom is replaced by a fluoro atom. Fluorinated variations can provide therapeutic advantages resulting from greater metabolic stability, e.g., increased in vivo half-life or reduced dosage requirements.

Pharmaceutically acceptable prodrugs of the compounds disclosed herein are contemplated and can be synthesized using conventional methods known in the art or methods corresponding to those described in the Examples (e.g., converting hydroxyl groups or carboxylic acid groups to ester groups). As used herein, a “prodrug” refers to a compound that can be converted via some chemical or physiological process (e.g., enzymatic processes and metabolic hydrolysis) to a therapeutic agent. Thus, the term “prodrug” also refers to a precursor of a biologically active compound that is pharmaceutically acceptable. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

Characterization of Exemplary Heterobifunctional Compounds

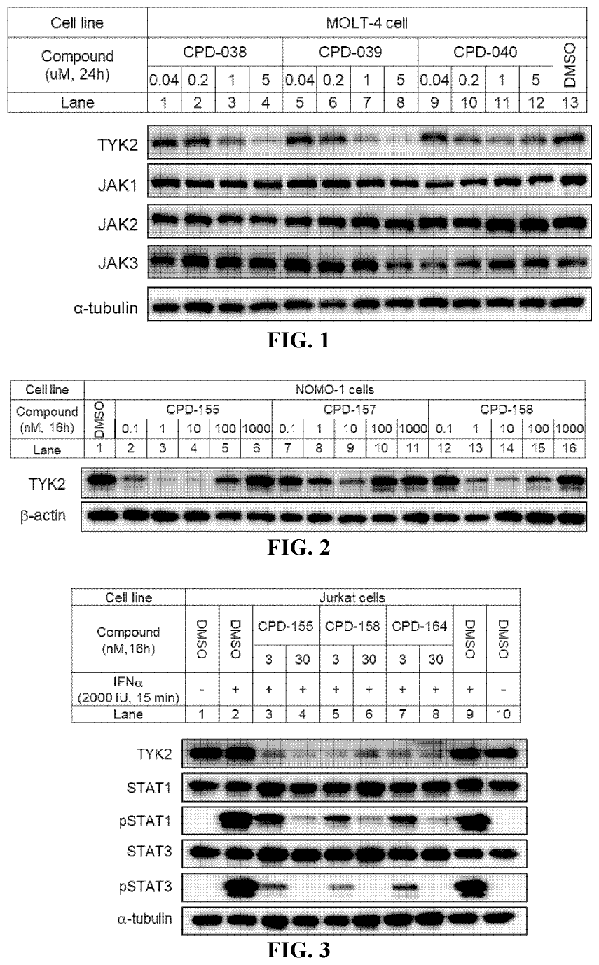

Synthesized heterobifunctional compounds were first characterized using immunoblotting assays. MOLT-4 cells were treated with bifunctional degraders at 0.5 and 5 μM concentration for 24 hours. Compounds CPD-038, CPD-039, and CPD-040 were able to significantly reduce TYK2 protein levels (Table 2). We further confirmed that CPD-038, CPD-039, and CPD-040 were able to reduce TYK2 protein levels in a concentration-dependent manner in MOLT-4 cells. More importantly, CPD-038, CPD-039, and CPD-040 are highly selective at the degradation of TYK2 over JAKT/2/3 proteins (FIG. 1). Our data further showed that CPD-155, CPD-157 and CPD-158 were highly potent in reducing TYK2 protein levels in NOMO-1 cells (FIG. 2; Table 3). Moreover, treatment with heterobifunctional compounds CPD-155, CPD-158 and CPD-164 significantly decreased STAT1/3 phosphorylation downstream of IFNα stimulation, which is correlated with the reduction of TYK2 protein levels in Jurkat cells (FIG. 3), suggesting a significant suppression of type I IFN immune response via TYK2 downregulation. Therefore, these selective TYK2 degraders could provide good therapeutic windows in clinic.

### Definition of Terms

As used herein, the terms “comprising” and “including” are used in their open, non-limiting sense.

As used herein, the term “heterobifunctional compound(s)” and “bivalent compound(s)” can be used interchangeably.

As used herein, the terms “Tyrosine Kinase 2 ligand” and “TYK2 ligand”, or “TYK2 targeting moiety” are to be construed to encompass any molecules ranging from small molecules to large proteins that associate with or bind to TYK2 proteins. The TYK2 ligand is capable of binding to a TYK2 protein comprising TYK2, a TYK2 mutant, a TYK2 deletion, or a TYK2 fusion protein. The TYK2 ligand can be, for example but not limited to, a small molecule compound (i.e., a molecule of molecular weight less than about 1.5 kilodaltons (kDa)), a peptide or polypeptide, nucleic acid or oligonucleotide, carbohydrate such as oligosaccharides, or an antibody or fragment thereof.

“Alkyl” refers to a straight or branched hydrocarbon chain radical consisting solely of carbon and hydrogen atoms, containing no unsaturation. An alkyl may comprise one, two, three, four, five, six, seven, eight, nine, ten, eleven, twelve, thirteen, fourteen, fifteen, or sixteen carbon atoms. In certain embodiments, an alkyl comprises one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈alkyl). The alkyl is attached to the rest of the molecule by a single bond, for example, methyl (Me), ethyl (Et), n-propyl (nPr), 1-methylethyl (iso-propyl, iPr), n-butyl, n-pentyl, 1,1-dimethylethyl (t-butyl), pentyl, 3-methylhexyl, 2-methylhexyl, and the like.

“Alkenyl” refers to a straight or branched hydrocarbon chain radical group consisting solely of carbon and hydrogen atoms, containing at least one double bond. An alkenyl may comprise two, three, four, five, six, seven, eight, nine, ten, eleven, twelve, thirteen, fourteen, fifteen, or sixteen carbon atoms. In certain embodiments, an alkenyl comprises two to twelve carbon atoms (e.g., C₂-C₁₂ alkenyl). In certain embodiments, an alkenyl comprises two to eight carbon atoms (e.g., C₂-C₈ alkenyl). In certain embodiments, an alkenyl comprises two to six carbon atoms (e.g., C₂-C₆ alkenyl). In other embodiments, an alkenyl comprises two to four carbon atoms (e.g., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. The term “allyl,” as used herein, means a —CH₂CH═CH₂ group.

As used herein, the term “alkynyl” refers to a straight or branched hydrocarbon chain radical group consisting solely of carbon and hydrogen atoms, containing at least one triple bond. An alkynyl may comprise two, three, four, five, six, seven, eight, nine, ten, eleven, twelve, thirteen, fourteen, fifteen, or sixteen carbon atoms. In certain embodiments, an alkynyl comprises two to twelve carbon atoms (e.g., C₂-C₁₂ alkynyl). In certain embodiments, an alkynyl comprises two to eight carbon atoms (e.g., C₂-C₈ alkynyl). In other embodiments, an alkynyl has two to six carbon atoms (e.g., C₂-C₆ alkynyl). In other embodiments, an alkynyl has two to four carbon atoms (e.g., C₂-C₄ alkynyl). The alkynyl is attached to the rest of the molecule by a single bond. Examples of such groups include, but are not limited to, ethynyl, propynyl, 1-butynyl, 2-butynyl, 1-pentynyl, 2-pentynyl, 1-hexynyl, 2-hexynyl, 3-hexynyl, and the like.

The term “heteroalkyl” by itself or in combination with another term, means, unless otherwise stated, a stable straight or branched chain, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —Si(CH₃)₃, and —CH₂—CH═N—OCH₃. Up to two heteroatoms may be consecutive, such as, for example, —CH₂—NH—OCH₃ and —CH₂—O—Si(CH₃)₃. Similarly, the terms “heteroalkenyl” and “heteroalkynyl” by itself or in combination with another term, means, unless otherwise stated, an alkenyl group or alkynyl group, respectively, that contains the stated number of carbons and having from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group.

The term “alkoxy”, as used herein, means an alkyl group as defined herein witch is attached to the rest of the molecule via an oxygen atom. Examples of such groups include, but are not limited to, methoxy, ethoxy, n-propyloxy, iso-propyloxy, n-butoxy, iso-butoxy, tert-butoxy, pentyloxy, hexyloxy, and the like.

The term “aryl”, as used herein, “refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon atoms. An aryl may comprise from six to eighteen carbon atoms, where at least one of the rings in the ring system is fully unsaturated, i.e., it contains a cyclic, delocalized (4n+2) π-electron system in accordance with the Hückel theory. In certain embodiments, an aryl comprises six to fourteen carbon atoms (C₆-C₁₄ aryl or 6-14 membered aryl). In certain embodiments, an aryl comprises six to ten carbon atoms (C₆-C₁₀ aryl or 6-10 membered aryl). Examples of such groups include, but are not limited to, phenyl, fluorenyl and naphthyl. The terms “Ph” and “phenyl,” as used herein, mean a —C₆H₅ group.

The term “heteroaryl”, refers to a radical derived from a 3- to 18-membered aromatic ring radical (i.e. 3-18 membered heteroaryl) that comprises two to seventeen carbon atoms and from one to six heteroatoms selected from nitrogen, oxygen and sulfur. As used herein, the heteroaryl radical may be a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. In certain embodiments, a heteroaryl refers to a radical derived from a 3- to 10-membered aromatic ring radical (3-10 membered heteroaryl). In certain embodiments, a heteroaryl refers to a radical derived from 5- to 7-membered aromatic ring (5-7 membered heteroaryl). Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of such groups include, but not limited to, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, furopyridinyl, and the like. In certain embodiments, a heteroaryl is attached to the rest of the molecule via a ring carbon atom. In certain embodiments, an heteroaryl is attached to the rest of the molecule via a nitrogen atom (N-attached) or a carbon atom (C-attached). For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached).

The term “heterocyclyl”, as used herein, means a non-aromatic, monocyclic, bicyclic, tricyclic, or tetracyclic radical having a total of from 4, 5, 6, 7, 8, 9, 10, 11, 12, 13, 14, 15, or 16 atoms in its ring system, and containing from 3 to 12 (such as 3, 4, 5, 6, 7, 8, 9, 10, 11, or 12)carbon atoms and from 1 to 4 (such as 1, 2. 3 or 4) heteroatoms each independently selected from O, S and N, and with the proviso that the ring of said group does not contain two adjacent O atoms or two adjacent S atoms. A heterocyclyl group may include fused, bridged or spirocyclic ring systems. In certain embodiments, a hetercyclyl group comprises 3 to 10 ring atoms (3-10 membered heterocyclyl). In certain embodiments, a hetercyclyl group comprises 3 to 8 ring atoms (3-8 membered heterocyclyl). In certain embodiments, a hetercyclyl group comprises 3 to 6 ring atoms (3-6 membered heterocyclyl). In certain embodiments, a hetercyclyl group comprises 4 to 6 ring atoms (4-6 membered heterocyclyl). A heterocyclyl group may contain an oxo substituent at any available atom that will result in a stable compound. For example, such a group may contain an oxo atom at an available carbon or nitrogen atom. Such a group may contain more than one oxo substituent if chemically feasible. In addition, it is to be understood that when such a heterocyclyl group contains a sulfur atom, said sulfur atom may be oxidized with one or two oxygen atoms to afford either a sulfoxide or sulfone. An example of a 4 membered heterocyclyl group is azetidinyl (derived from azetidine). An example of a 5 membered cycloheteroalkyl group is pyrrolidinyl. An example of a 6 membered cycloheteroalkyl group is piperidinyl. An example of a 9 membered cycloheteroalkyl group is indolinyl. An example of a 10 membered cycloheteroalkyl group is 4H-quinolizinyl. Further examples of such heterocyclyl groups include, but are not limited to, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, 3-oxopiperazinyl, 4-methylpiperazinyl, 4-ethylpiperazinyl, and 1-oxo-2,8-diazaspiro[4.5]dec-8-yl. A heteroaryl group may be attached to the rest of molecular via a carbon atom (C-attached) or a nitrogen atom (N-attached). For instance, a group derived from piperazine may be piperazin-1-yl (N-attached) or piperazin-2-yl (C-attached).

The term “cycloalkyl” or “carbocyclyl” means a saturated, monocyclic, bicyclic, tricyclic, or tetracyclic radical having a total of from 3, 4, 5, 6, 7, 8, 9, 10, 11, 12, or 13 carbon atoms in its ring system. A cycloalkyl may be fused, bridged or spirocyclic. In certain embodiments, a cycloalkyl comprises 3 to 8 carbon ring atoms (3-8 membered or C₃-C₈carbocyclyl). In certain embodiments, a cycloalkyl comprises 3 to 10 carbon ring atoms (C₃-C₁₀ cycloalkyl). Examples of such groups include, but are not limited to, cyclopropyl(cPr), cyclobutyl, cyclopentyl, cyclopentenyl, cyclohexyl, cycloheptyl, adamantyl, and the like.

The term “spirocyclic” as used herein has its conventional meaning, that is, any ring system containing two or more rings wherein two of the rings have one ring carbon in common. Each ring of the spirocyclic ring system, as herein defined, independently comprises 3 to 20 ring atoms. Preferably, they have 3 to 10 ring atoms. Non-limiting examples of a spirocyclic system include spiro[3.3]heptane, spiro[3.4]octane, and spiro[4.5]decane.

The term cyano” refers to a —C≡N group.

An “aldehyde” group refers to a —C(O)H group.

An “alkoxy” group refers to both an —O-alkyl, as defined herein.

An “alkoxycarbonyl” refers to a —C(O)-alkoxy, as defined herein.

An “alkylaminoalkyl” group refers to an -alkyl-NR-alkyl group, as defined herein.

An “alkylsulfonyl” group refer to a —SO₂alkyl, as defined herein.

An “amino” group refers to an optionally substituted —NH₂.

An “aminoalkyl” group refers to an -alkyl-amino group (such as —CH₂(NH₂)), as defined herein.

An “alkylamino” group refers to an -amino-alkyl group (such as —NH(CH₃)), as defined herein.

An “cycloalkylamino” group refers to an -amino-cycloalkyl group (such as

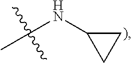 [SMILES: *NC1CC1]

as defined herein.

An “aminocarbonyl” refers to a —C(O)-amino, as defined herein.

An “arylalkyl” group refers to -alkylaryl, where alkyl and aryl are defined herein.

An “aryloxy” group refers to both an —O-aryl and an —O-heteroaryl group, as defined herein.

An “aryloxycarbonyl” refers to —C(O)-aryloxy, as defined herein.

An “arylsulfonyl” group refers to a —SO₂aryl, as defined herein.

A “carbonyl” group refers to a —C(O)— group, as defined herein.

A “carboxylic acid” group refers to a —C(O)OH group.

A “cycloalkoxy” refers to a —O-cycloalkyl group, as defined herein.

A “halo” or “halogen” group refers to fluorine, chlorine, bromine or iodine.

A “haloalkyl” group refers to an alkyl group substituted with one or more halogen atoms.

A “hydroxy” group refers to an —OH group.

A “nitro” group refers to a —NO₂ group.

An “oxo” group refers to the ═O substituent.

A “trihalomethyl” group refers to a methyl substituted with three halogen atoms.

The term “alkylene” is a bidentate radical obtained by removing a hydrogen atom from a alkyl group as defined above. Examples of such groups include, but are not limited to, —CH₂—, —CH₂CH₂—, etc. The term “cycloalkylene” or “carbocyclylene” is a bidentate radical obtained by removing a hydrogen atom from a cycloalkyl ring as defined above. Examples of such groups include, but are not limited to, cyclopropylene, cyclobutylene, cyclopentylene, cyclopentenylene, cyclohexylene, cycloheptylene, and the like. Similarly, the terms “alkenylene”, “alkynylene”, “alkoxyalkylene”, “haloalkylene”, “hydroxyalkylene”, “aminoalkylene”, “alkylaminoalkylene”, and “heterocyclylene” are bidentate radicals obtained by removing a hydrogen atom from an alkenyl radical, an alkynyl radical, an alkoxyalkyl radical, a haloalkyl radical, an hydroxyalkylene”, “aminoalkyl radical, and an alkylaminoalkyl radical, respectively.

The term “heteroalkylene” by itself or in combination with another term, means, unless otherwise stated, a straight or branched divalent radical group, derived from heteroalkyl, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₂—, —CH₂—CH₂—NH—CH₂—, —CH₂—CH₂—N(CH₃)—CH₂—, —CH₂—S—CH₂—CH₂—, —CH₂—CH₂—S(O)—CH₂—, —CH₂—CH₂—S(O)₂—CH₂—, —CH(Si(CH₃)₃)—CH₂—, and —CH₂—CH═N—OCH₂—. Up to two heteroatoms may be consecutive, such as, for example, —CH₂—NH—OCH₂— and —CH(O—Si(CH₃)₃)—CH₂—. Similarly, the terms “heteroalkenylene” and “heteroalkynylene” by itself or in combination with another term, means, unless otherwise stated, an alkenylene group or alkynylene group, respectively, that contains the stated number of carbons and having from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group.

The term “length” when refers to a moiety means the smallest number of carbon and/or hetero atoms from one end to the other end of the moiety. When it refers to the linker, it means the smallest number of atoms from the end connects to the TRK ligand and the end connects to the degradation tag. It applies to both situations where the linker is linear or branched, and where the linker comprises a ring system.

The term “substituted”, unless otherwise stated, means that the specified group or moiety bears one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “null” means the absence of an atom or moiety, and there is a bond between adjacent atoms in the structure.

The term “optionally substituted” means that the specified group may be either unsubstituted or substituted by one or more substituents as defined herein. It is to be understood that in the compounds of the present invention when a group is said to be “unsubstituted,” or is “substituted” with fewer groups than would fill the valencies of all the atoms in the compound, the remaining valencies on such a group are filled by hydrogen. For example, if a C₆ aryl group, also called “phenyl” herein, is substituted with one additional substituent, one of ordinary skill in the art would understand that such a group has 4 open positions left on carbon atoms of the C₆ aryl ring (6 initial positions, minus one at which the remainder of the compound of the present invention is attached to and an additional substituent, remaining 4 positions open). In such cases, the remaining 4 carbon atoms are each bound to one hydrogen atom to fill their valencies. Similarly, if a C₆ aryl group in the present compounds is said to be “disubstituted,” one of ordinary skill in the art would understand it to mean that the C₆ aryl has 3 carbon atoms remaining that are unsubstituted. Those three unsubstituted carbon atoms are each bound to one hydrogen atom to fill their valencies. Unless otherwise specified, an optionally substituted radical may be a radical unsubstituted or substituted with one or more substituents selected from halogen, CN, NO₂, OR^{m}, SR^{m}, NR^{m}R^{o}, COR^{m}, CO₂R^{m}, CONR^{m}R^{o}, SOR^{m}, SO₂R^{m}, SO₂NR^{m}R^{o}, NR^{m}COR^{o}, NR^{m}C(O)NR^{m}R^{o}, NR^{m}SOR^{o}, NR^{m}SO₂R^{o}, C₁-C₈ alkyl, C₁-C₈alkoxyC₁-C₈alkyl, C₁-C₈ haloalkyl, C₁-C₈ hydroxyalkyl, C₁-C₈alkylaminoC₁-C₈ alkyl, C₃-C₇ cycloalkyl, 3-7 membered heterocyclyl, C₂-C₈ alkenyl, C₂-C₈ alkynyl, aryl, and heteroaryl, wherein R^{m}, Rⁿ, and R^{o} are independently selected from null, hydrogen, C₁-C₈ alkyl, C₂-C₈ alkenyl, C₂-C₈ alkynyl, C₃-C₇ cycloalkyl, 3-7 membered heterocyclyl, aryl, and heteroaryl, or Rⁿ and R^{o} together with the atom to which they are connected form a 3-8 membered cycloalkyl or heterocyclyl ring.

As used herein, the same symbol in different FORMULA means different definition, for example, the definition of R1 in FORMULA 1 is as defined with respect to FORMULA 1 and the definition of R1 in FORMULA 6 is as defined with respect to FORMULA 6.

As used herein, each unit in the linker moiety

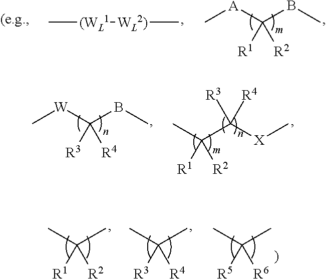

can be the same as or different from each other. In certain embodiments, each unit in the linker moiety is the same as each other.

As used herein, when m (or n or o or p) is defined by a range, for example, “m is 0 to 15” or “m=0-3” mean that m is an integer from 0 to 15 (i.e. m is 0, 1, 2, 3, 4, 5, 6, 7, 8, 9, 10, 11, 12, 13, 14, or 15) or m is an integer from 0 to 3 (i.e. m is 0, 1,2, or 3) or is any integer in the defined range.

“Pharmaceutically acceptable salt” includes both acid and base addition salts. A pharmaceutically acceptable salt of any one of the heterobifunctional compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

Pharmaceutical Compositions

In some aspects, the compositions and methods described herein include the manufacture and use of pharmaceutical compositions and medicaments that include one or more heterobifunctional compounds as disclosed herein. Also included are the pharmaceutical compositions themselves.

In some aspects, the compositions disclosed herein can include other compounds, drugs, or agents used for the treatment of cancer. For example, in some instances, pharmaceutical compositions disclosed herein can be combined with one or more (e.g., one, two, three, four, five, or less than ten) compounds. Such additional compounds can include, e.g., conventional chemotherapeutic agents or any other cancer treatment known in the art. When co-administered, heterobifunctional compounds disclosed herein can operate in conjunction with conventional chemotherapeutic agents or any other cancer treatment known in the art to produce mechanistically additive or synergistic therapeutic effects.

In some aspects, the pH of the compositions disclosed herein can be adjusted with pharmaceutically acceptable acids, bases, or buffers to enhance the stability of the heterobifunctional compound or its delivery form.

Pharmaceutical compositions typically include a pharmaceutically acceptable excipient, adjuvant, or vehicle. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human. A pharmaceutically acceptable excipient, adjuvant, or vehicle is a substance that can be administered to a patient, together with a compound of the invention, and which does not compromise the pharmacological activity thereof and is nontoxic when administered in doses sufficient to deliver a therapeutic amount of the compound. Exemplary conventional nontoxic pharmaceutically acceptable excipients, adjuvants, and vehicles include, but not limited to, saline, solvents, dispersion media, coatings, antibacterial and antifungal agents, isotonic and absorption delaying agents, and the like, compatible with pharmaceutical administration.

In particular, pharmaceutically acceptable excipients, adjuvants, and vehicles that can be used in the pharmaceutical compositions of this invention include, but are not limited to, ion exchangers, alumina, aluminum stearate, lecithin, self-emulsifying drug delivery systems (SEDDS) such as d-a-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of compounds of the formulae described herein.

Depending on the dosage form selected to deliver the heterobifunctional compounds disclosed herein, different pharmaceutically acceptable excipients, adjuvants, and vehicles may be used. In the case of tablets for oral use, pharmaceutically acceptable excipients, adjuvants, and vehicles may be used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient may be suspended or dissolved in an oily phase is combined with emulsifying or suspending agents. If desired, certain sweetening, flavoring, or coloring agents can be added.

As used herein, the heterobifunctional compounds disclosed herein are defined to include pharmaceutically acceptable derivatives or prodrugs thereof. A “pharmaceutically acceptable derivative” means any pharmaceutically acceptable salt, solvate, or prodrug, e.g., carbamate, ester, phosphate ester, salt of an ester, or other derivative of a compound or agent disclosed herein, which upon administration to a recipient is capable of providing (directly or indirectly) a compound described herein, or an active metabolite or residue thereof. Particularly favored derivatives and prodrugs are those that increase the bioavailability of the compounds disclosed herein when such compounds are administered to a subject (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group that enhances aqueous solubility or active transport through the gut membrane is appended to the structure of formulae described herein. Such derivatives are recognizable to those skilled in the art without undue experimentation. Nevertheless, reference is made to the teaching of Burger's Medicinal Chemistry and Drug Discovery, 5^{th} Edition, Vol. 1: Principles and Practice, which is incorporated herein by reference to the extent of teaching such derivatives.

The heterobifunctional compounds disclosed herein include pure enantiomers, mixtures of enantiomers, pure diastereoisomers, mixtures of diastereoisomers, diastereoisomeric racemates, mixtures of diastereoisomeric racemates and the meso-form and pharmaceutically acceptable salts, solvent complexes, morphological forms, or deuterated derivatives thereof.

In some aspects, the pharmaceutical compositions disclosed herein can include an effective amount of one or more heterobifunctional compounds. The terms “effective amount” and “effective to treat,” as used herein, refer to an amount or a concentration of one or more compounds or a pharmaceutical composition described herein utilized for a period of time (including acute or chronic administration and periodic or continuous administration) that is effective within the context of its administration for causing an intended effect or physiological outcome (e.g., treatment or prevention of cell growth, cell proliferation, or cancer). In some aspects, pharmaceutical compositions can further include one or more additional compounds, drugs, or agents used for the treatment of cancer (e.g., conventional chemotherapeutic agents) in amounts effective for causing an intended effect or physiological outcome (e.g., treatment or prevention of cell growth, cell proliferation, or cancer).

In some aspects, the pharmaceutical compositions disclosed herein can be formulated for sale in the United States, import into the United States, or export from the United States.

Administration of Pharmaceutical Compositions

The pharmaceutical compositions disclosed herein can be formulated or adapted for administration to a subject via any route, e.g., any route approved by the Food and Drug Administration (FDA). Exemplary methods are described in the FDA Data Standards Manual (DSM) (available at http://www.fda.gov/Drugs/DevelopmentApprovalProcess/FormsSubmissionRequirements/ElectronicSubmissions/DataStandardsManualmonographs). In particular, the pharmaceutical compositions can be formulated for and administered via oral, parenteral, or transdermal delivery. The term “parenteral” as used herein includes subcutaneous, intracutaneous, intravenous, intramuscular, intraperitoneal, intra-articular, intra-arterial, intrasynovial, intrasternal, intrathecal, intralesional, and intracranial injection or infusion techniques.

For example, the pharmaceutical compositions disclosed herein can be administered, e.g., topically, rectally, nasally (e.g., by inhalation spray or nebulizer), buccally, vaginally, subdermally (e.g., by injection or via an implanted reservoir), or ophthalmically.

For example, pharmaceutical compositions of this invention can be orally administered in any orally acceptable dosage form including, but not limited to, capsules, tablets, emulsions and aqueous suspensions, dispersions and solutions.

For example, the pharmaceutical compositions of this invention can be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax, and polyethylene glycols.

For example, the pharmaceutical compositions of this invention can be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, or other solubilizing or dispersing agents known in the art.

For example, the pharmaceutical compositions of this invention can be administered by injection (e.g., as a solution or powder). Such compositions can be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, e.g., as a solution in 1,3-butanediol. Among the acceptable vehicles and solvents that may be employed are mannitol, water, Ringers solution, and isotonic sodium chloride solution. In addition, sterile, fixed oils are conventionally employed as a solvent or suspending medium. For this purpose, any bland fixed oil can be employed, including synthetic mono- or diglycerides. Fatty acids, such as oleic acid and its glyceride derivatives are useful in the preparation of injectables, as are natural pharmaceutically-acceptable oils, e.g., olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens, Spans, or other similar emulsifying agents or bioavailability enhancers which are commonly used in the manufacture of pharmaceutically acceptable solid, liquid, or other dosage forms can also be used for the purposes of formulation.

In some aspects, an effective dose of a pharmaceutical composition of this invention can include, but is not limited to, e.g., about 0.00001, 0.0001, 0.001, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.5, 3, 4, 5, 6, 7, 8, 9, 10, 15, 20, 30, 40, 50, 60, 70, 80, 90, 100, 200, 300, 400, 500, 600, 700, 800, 900, 1000, 2500, 5000, or 10000 mg/kg/day, or according to the requirements of the particular pharmaceutical composition.

When the pharmaceutical compositions disclosed herein include a combination of the heterobifunctional compounds described herein and one or more additional compounds (e.g., one or more additional compounds, drugs, or agents used for the treatment of cancer or any other condition or disease, including conditions or diseases known to be associated with or caused by cancer, inflammation, and/or autoimmune diseases), both the heterobifunctional compounds and the additional compounds may be present at dosage levels of between about 1 to 100%, and more preferably between about 5 to 95% of the dosage normally administered in a monotherapy regimen. The additional agents can be administered separately, as part of a multiple dose regimen, from the compounds of this invention. Alternatively, those agents can be part of a single dosage form, mixed together with the compounds of this invention in a single composition.

In some aspects, the pharmaceutical compositions disclosed herein can be included in a container, pack, or dispenser together with instructions for administration.

Methods of Treatment

The methods disclosed herein contemplate administration of an effective amount of a compound or composition to achieve the desired or stated effect. Typically, the compounds or compositions of the invention will be administered from about 1 to about 6 times per day or, alternately or in addition, as a continuous infusion. Such administration can be used as a chronic or acute therapy. The amount of active ingredient that can be combined with the carrier materials to produce a single dosage form will vary depending upon the host treated and the particular mode of administration. A typical preparation will contain from about 5% to about 95% active compound (w/w). Alternatively, such preparations can contain from about 20% to about 80% active compound.

In some aspects, provided herein are a heterobifunctional compound described herein for preventing or treating a disease or condition.

In some aspects, provided herein are a heterobifunctional compound described herein for treating or preventing one or more diseases or conditions disclosed herein in a subject in need thereof. In certain embodiments, the disease or condition is a TYK2-mediated disease or condition. In certain embodiments, the disease or condition is resulted from TYK2 expression, mutation, deletion, or fusion. In certain embodiments, the diseases or conditions are cancer, inflammation, auto-immune disease, viral infections, and immunological diseases. In one embodiment, the TYK2-mediated cancer is selected from the group consisting of brain cancer, stomach cancer, gastrointestinal tract cancer, liver cancer, biliary passage cancer, breast cancer, ovary cancer, cervix cancer, prostate cancer, testis cancer, penile cancer, genitourinary tract cancer, esophagus cancer, larynx cancer, skin cancer, lung cancer, pancreas cancer, thyroid cancer, gland cancer, bladder cancer, kidney cancer, muscle cancer, bone cancer, cancers of the hematopoietic system, myeloproliferative neoplasms, essential thrombocythemia, polycythemia vera, primary myelofibrosis, chronic neutrophilic leukemia, acute lymphoblastic leukemia, Hodgkin's lymphoma, chronic myelomonocytic leukemia, systemic mast cell disease, hyper eosinophilic syndrome, cutaneous T-cell lymphoma, B-cell lymphoma, and myeloma. In one embodiment, the TYK2-mediated inflammatory disorders are selected from the group consisting of ankylosing spondylitis, Crohn's disease, inflammatory bowel disease, ulcerative colitis, and ischemia reperfusion injuries. In one embodiment, the TYK2-mediated auto-immune diseases are selected from the group consisting of multiple sclerosis, rheumatoid arthritis, psoriatic arthritis, juvenile idiopathic arthritis, psoriasis, myasthenia gravis, type I diabetes, systemic lupus erythematosus, IgA nephropathy, autoimmune thyroid disorders, alopecia areata, and bullous pemphigoid. In one embodiment, the TYK2-mediated dermatological disorders are selected from the group consisting of atopic dermatitis, pruritus, alopecia areata, psoriasis, skin rash, skin irritation, skin sensitization, chronic mucocutaneous candidiasis, dermatomyositis, erythema multiforme, palmoplantar pustulosis, vitiligo, polyarteritis nodosa, and STING-associated vasculopathy. In one embodiment, the TYK2-mediated viral infections are selected from the group consisting of infections of Hepatitis B, Hepatitis C, Human Immunodeficiency Virus (HIV), Human T-lymphotropic Virus (HTLV1), Epstein Barr Virus (EBV), Varicella-Zoster Virus (VZV) and Human Papilloma Virus (HPV). In one embodiment, the TYK2-mediated dry eye disorders are selected from the group consisting of dry eye syndrome (DES) and keratoconjunctivitis sicca (KCS). In one embodiment, the TYK2-mediated bone remodeling disorders are selected from the group consisting of osteoporosis and osteoarthritis. In one embodiment, the TYK2-mediated organ transplant associated immunological complications are selected from the group consisting of graft-versus-host diseases.

In some aspects, provided herein are use of a heterobifunctional compound in manufacture of a medicament for preventing or treating one or more diseases or conditions disclosed herein.

In some aspects, the methods disclosed include the administration of a therapeutically effective amount of one or more of the compounds or compositions described herein to a subject (e.g., a mammalian subject, e.g., a human subject) who is in need of, or who has been determined to be in need of, such treatment. In some aspects, the methods disclosed include selecting a subject and administering to the subject an effective amount of one or more of the compounds or compositions described herein, and optionally repeating administration as required for the prevention or treatment of cancer.

In some aspects, subject selection can include obtaining a sample from a subject (e.g., a candidate subject) and testing the sample for an indication that the subject is suitable for selection. In some aspects, the subject can be confirmed or identified, e.g. by a health care professional, as having had, having an elevated risk to have, or having a condition or disease. In some aspects, suitable subjects include, for example, subjects who have or had a condition or disease but that resolved the disease or an aspect thereof, present reduced symptoms of disease (e.g., relative to other subjects (e.g., the majority of subjects) with the same condition or disease), or that survive for extended periods of time with the condition or disease (e.g., relative to other subjects (e.g., the majority of subjects) with the same condition or disease), e.g., in an asymptomatic state (e.g., relative to other subjects (e.g., the majority of subjects) with the same condition or disease). In some aspects, exhibition of a positive immune response towards a condition or disease can be made from patient records, family history, or detecting an indication of a positive immune response. In some aspects, multiple parties can be included in subject selection. For example, a first party can obtain a sample from a candidate subject and a second party can test the sample. In some aspects, subjects can be selected or referred by a medical practitioner (e.g., a general practitioner). In some aspects, subject selection can include obtaining a sample from a selected subject and storing the sample or using the in the methods disclosed herein. Samples can include, e.g., cells or populations of cells.

In some aspects, methods of treatment can include a single administration, multiple administrations, and repeating administration of one or more compounds disclosed herein as required for the prevention or treatment of the disease or condition disclosed herein (e.g., an TYK2-mediated disease). In some aspects, methods of treatment can include assessing a level of disease in the subject prior to treatment, during treatment, or after treatment. In some aspects, treatment can continue until a decrease in the level of disease in the subject is detected.

The term “subject,” as used herein, refers to any animal. In some instances, the subject is a mammal. In some instances, the term “subject,” as used herein, refers to a human (e.g., a man, a woman, or a child).

The terms “administer,” “administering,” or “administration,” as used herein, refer to implanting, ingesting, injecting, inhaling, or otherwise absorbing a compound or composition, regardless of form. For example, the methods disclosed herein include administration of an effective amount of a compound or composition to achieve the desired or stated effect.

The terms “treat”, “treating,” or “treatment,” as used herein, refer to partially or completely alleviating, inhibiting, ameliorating, or relieving the disease or condition from which the subject is suffering. This means any manner in which one or more of the symptoms of a disease or disorder (e.g., cancer) are ameliorated or otherwise beneficially altered. As used herein, amelioration of the symptoms of a particular disorder (e.g., cancer) refers to any lessening, whether permanent or temporary, lasting or transient that can be attributed to or associated with treatment by the heterobifunctional compounds, compositions and methods of the present invention. In some embodiments, treatment can promote or result in, for example, a decrease in the number of tumor cells (e.g., in a subject) relative to the number of tumor cells prior to treatment; a decrease in the viability (e.g., the average/mean viability) of tumor cells (e.g., in a subject) relative to the viability of tumor cells prior to treatment; a decrease in the rate of growth of tumor cells; a decrease in the rate of local or distant tumor metastasis; or reductions in one or more symptoms associated with one or more tumors in a subject relative to the subject's symptoms prior to treatment.

The terms “prevent,” “preventing,” and “prevention,” as used herein, shall refer to a decrease in the occurrence of a disease or decrease in the risk of acquiring a disease or its associated symptoms in a subject. The prevention may be complete, e.g., the total absence of disease or pathological cells in a subject. The prevention may also be partial, such that the occurrence of the disease or pathological cells in a subject is less than, occurs later than, or develops more slowly than that which would have occurred without the present invention. In certain embodiments, the subject has an elevated risk of developing one or more TYK2-mediated diseases. Exemplary TYK2-mediated diseases that can be treated with heterobifunctional compounds include, for example, cancer (e.g. cancers of brain, stomach, gastrointestinal tracts, liver, biliary passage, breast, ovary, cervix, prostate, testis, penile, genitourinary tract, esophagus, larynx, skin, lung, pancreas, thyroid, glands, bladder, kidney, muscle, bone, and cancers of the hematopoietic system, such as myeloproliferative neoplasms, including essential thrombocythemia, polycythemia vera, primary myelofibrosis, chronic neutrophilic leukemia, acute lymphoblastic leukemia, Hodgkin's lymphoma, chronic myelomonocytic leukemia, systemic mast cell disease, hypereosinophilic syndrome, cutaneous T-cell lymphoma, B-cell lymphoma, myeloma, and other hematologic malignancies, particularly cancers that involve inflammation, mutations or other aberrations that activate the TYK2 pathway); inflammation (e.g. ankylosing spondylitis, Crohn's disease, inflammatory bowel disease, ulcerative colitis, and ischemia reperfusion injuries, which are conditions related to inflammatory ischemic events such as stroke or cardiac arrest); auto-immune diseases (e.g. multiple sclerosis, rheumatoid arthritis, psoriatic arthritis, juvenile idiopathic arthritis, psoriasis, myasthenia gravis, type I diabetes, systemic lupus erythematosus, IgA nephropathy, autoimmune thyroid disorders, alopecia areata, and bullous pemphigoid); dermatological disorders (e.g. atopic dermatitis, pruritus, alopecia areata, psoriasis, skin rash, skin irritation, skin sensitization, chronic mucocutaneous candidiasis, dermatomyositis, erythema multiforme, palmoplantar pustulosis, vitiligo, polyarteritis nodosa, and STING-associated vasculopathy); viral infections (e.g. viral infections and consequent complications, such as infections of Hepatitis B, Hepatitis C, Human Immunodeficiency Virus (HIV), Human T-lymphotropic Virus (HTLV1), Epstein Barr Virus (EBV), Varicella-Zoster Virus (VZV) and Human Papilloma Virus (HPV)); dry eye disorder, also known as dry eye syndrome (DES) or keratoconjunctivitis sicca (KCS); bone remodeling disorders (e.g. osteoporosis and osteoarthritis); organ transplant associated immunological complications (e.g. graft-versus-host diseases).

Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds selected. Moreover, treatment of a subject with a therapeutically effective amount of the compounds or compositions described herein can include a single treatment or a series of treatments. For example, effective amounts can be administered at least once. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health or age of the subject, and other diseases present.

Following administration, the subject can be evaluated to detect, assess, or determine their level of disease. In some instances, treatment can continue until a change (e.g., reduction) in the level of disease in the subject is detected. Upon improvement of a patient's condition (e.g., a change (e.g., decrease) in the level of disease in the subject), a maintenance dose of a compound, or composition disclosed herein can be administered, if necessary. Subsequently, the dosage or frequency of administration, or both, can be reduced, e.g., as a function of the symptoms, to a level at which the improved condition is retained. Patients may, however, require intermittent treatment on a long-term basis upon any recurrence of disease symptoms.

The present disclosure is also described and demonstrated by way of the following examples. However, the use of these and other examples anywhere in the specification is illustrative only and in no way limits the scope and meaning of the invention or of any exemplified term. Likewise, the invention is not limited to any particular preferred embodiment or aspect described herein. Indeed, many modifications and variations may be apparent to those skilled in the art upon reading this specification, and such variations can be made without departing from the invention in spirit or in scope. The invention is therefore to be limited only by the terms of the appended claims along with the full scope of equivalents to which those claims are entitled.

### EXAMPLES

### Example 1: (2-(2,6-Dioxopiperidin-3-yl)-1,3-dioxoisoindolin-4-yl)glycine (Handle 1)

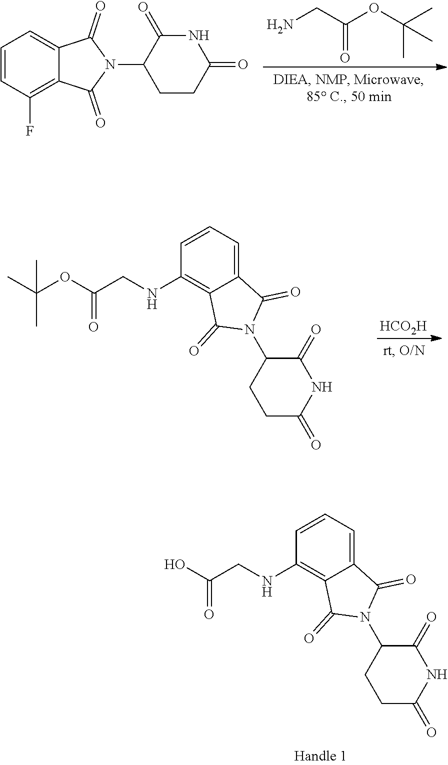

### Step 1. Synthesis of tert-butyl (2-(2,6-dioxopiperidin-3-yl)-1,3-dioxoisoindolin-4-yl)glycinate

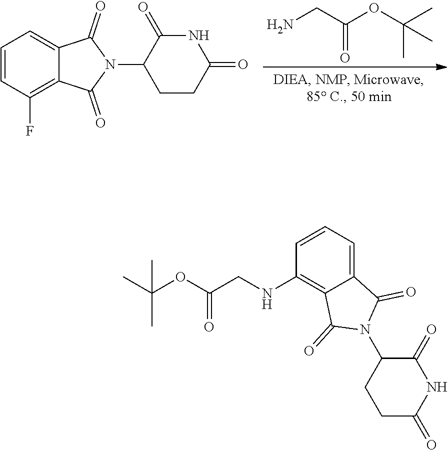

A solution of 2-(2,6-dioxopiperidin-3-yl)-4-fluoroisoindoline-1,3-dione (1.93 g, 7.0 mmol), tert-butyl 2-aminoacetate (1.01 g, 7.7 mmol) and N,N-diisopropylethylamine (2.72 g, 21 mmol) in NMP (14 mL) was heated to 85° C. in a microwave reactor for 50 min. Two batches were combined and diluted with EtOAc (100 mL), washed with water and brine, dried over anhydrous sodium sulfate and concentrated under reduced pressure. The resulting residue was purified by silica gel chromatography (eluted with hexanes/EtOAc=2:1) to give the title compound (1.0 g, yield: 18%) as a yellow solid. MS (ESI) m/z=332.0 [M−55]⁺.

### Step 2. Synthesis of (2-(2,6-Dioxopiperidin-3-yl)-1,3-dioxoisoindolin-4-yl)glycine

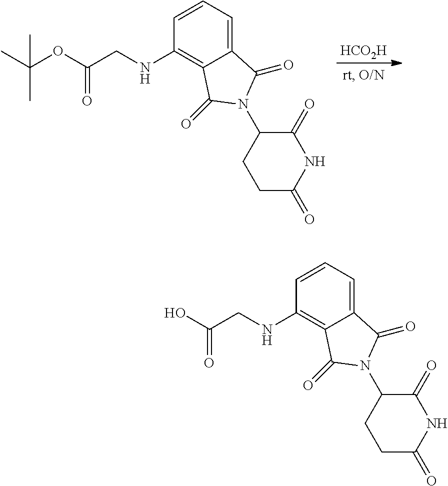

A solution of tert-butyl 2-((2-(2,6-dioxopiperidin-3-yl)-1,3-dioxoisoindolin-4-yl)amino)acetate (1.0 g, 2.58 mmol) in HCOOH (88%, 10 mL) was stirred at rt overnight. The reaction was concentrated and triturated with DCM, filtered, washed with DCM and MTBE, dried to give the title compound (840 mg, yield: 98%) as a yellow solid. ¹H NMR (400 MHz, DMSO-d6) δ 11.07 (s, 1H), 7.52 (t, J=7.6 Hz, 1H), 6.99-6.88 (m, 3H), 5.04 (dd, J=5.2, 12.8 Hz, 1H), 3.73 (s, 2H), 2.93-2.83 (m, 1H), 2.61-2.50 (m, 2H), 2.02 (t, J=5.6 Hz, JH). MS (ESI) m/z=330.1 [M−H]⁻.

### Example 2: 3-((2-(2,6-Dioxopiperidin-3-yl)-1,3-dioxoisoindolin-4-yl)amino)propanoic acid (Handle 2)

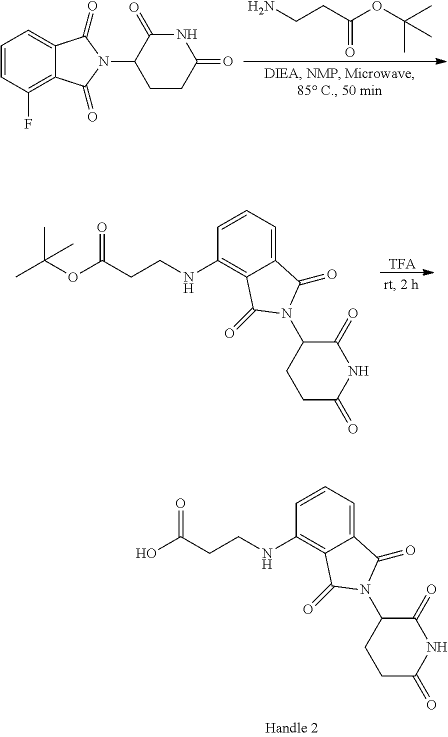

Handle 2 was synthesized following the same procedures as Handle 1 as described in Example 1 (1.42 g, yield: 24% over 2 steps). ¹H NMR (400 MHz, DMSO-d₆) δ 11.61 (br, 1H), 11.08 (s, 1H), 7.58 (dd, J=7.2, 8.8 Hz, 1H), 7.15 (d, J=8.8 Hz, 1H), 7.04 (d, J=7.2 Hz, 1H), 6.64 (s, 1H), 5.05 (dd, J=5.2, 12.8 Hz, 1H), 3.53 (t, J=6.4 Hz, 2H), 2.92-2.83 (m, 1H), 2.61-2.50 (m, 4H), 2.05-2.00 (m, 1H). MS (ESI) m/z=346.1 [M+H]⁺.

### Example 3: 4-((2-(2,6-Dioxopiperidin-3-yl)-1,3-dioxoisoindolin-4-yl)amino)butanoic acid (Handle 3)

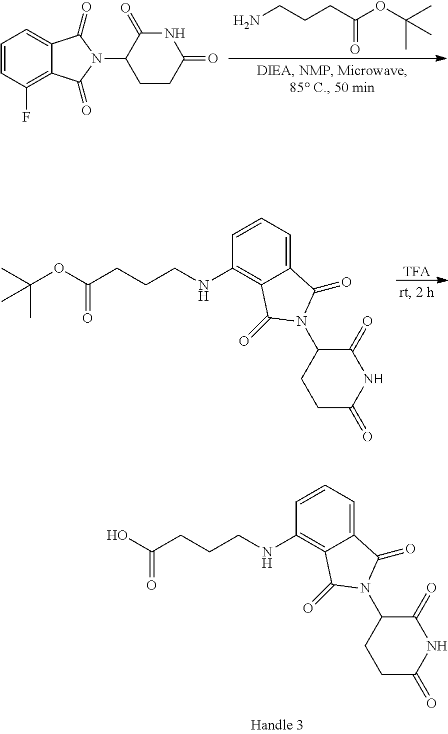 [SMILES: O=C1CCC(N2C(=O)c3cccc(F)c3C2=O)C(=O)N1 ; CC(C)(C)OC(=O)CCCN ; CC(C)(C)OC(=O)CCCNc1cccc2c1C(=O)N(C1CCC(=O)NC1=O)C2=O ; O=C(O)CCCNc1cccc2c1C(=O)N(C1CCC(=O)NC1=O)C2=O ; "TFA"]

Handle 3 was synthesized following the same procedures as Handle 1 as described in Example 1 (1.27 g, yield: 13% over 2 steps). ¹H NMR (400 MHz, DMSO-d₆) δ 12.12 (br, 1H), 11.08 (s, 1H), 7.58 (dd, J=7.2, 8.8 Hz, 1H), 7.13 (d, J=8.8 Hz, 1H), 7.03 (d, J=7.2 Hz, 1H), 6.64 (t, J=6.0 Hz, 1H), 5.05 (dd, J=5.6, 12.8 Hz, 1H), 3.33 (q, J=6.8 Hz, 2H), 2.93-2.83 (m, 1H), 2.61-2.50 (m, 2H), 2.31 (t, J=6.8 Hz, 2H), 2.07-2.00 (m, 1H), 1.83-1.75 (m, 2H). MS (ESI) m/z=360.1 [M+H]⁺.

### Example 4: 5-((2-(2,6-Dioxopiperidin-3-yl)-1,3-dioxoisoindolin-4-yl)amino)pentanoic acid (Handle 4)

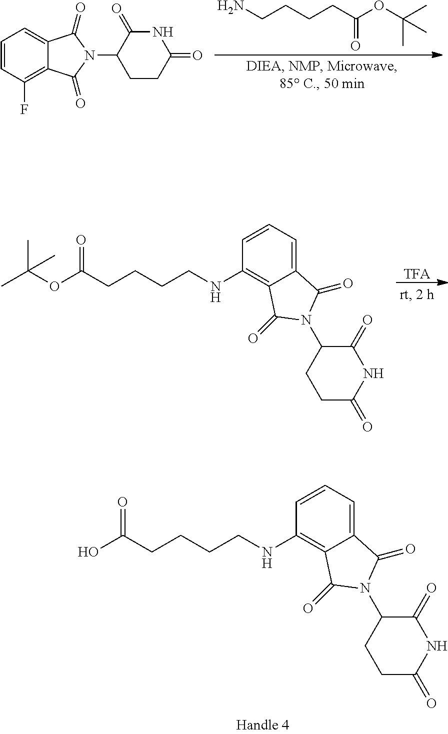 [SMILES: O=C1CCC(N2C(=O)c3cccc(F)c3C2=O)C(=O)N1 ; CC(C)(C)OC(=O)CCCCN ; CC(C)(C)OC(=O)CCCCNc1cccc2c1C(=O)N(C1CCC(=O)NC1=O)C2=O ; O=C(O)CCCCNc1cccc2c1C(=O)N(C1CCC(=O)NC1=O)C2=O ; "TFA"]

Handle 4 was synthesized following the same procedures as Handle 1 as described in Example 1 (1.4 g, yield: 15% over 2 steps). ¹H NMR (400 MHz, DMSO-d₆) δ 12.02 (brs, 1H), 11.08 (s, 1H), 7.58 (dd, J=8.8, 7.2 Hz, 1H), 7.10 (d, J=8.4 Hz, 1H), 7.02 (d, J=7.2 Hz, 1H), 6.64 (t, J=5.6 Hz, 1H), 5.07-5.03 (m, 1H), 3.32-3.02 (m, 2H), 2.93-2.84 (m, 1H), 2.61-2.54 (m, 2H), 2.28-2.25 (m, 2H), 2.05-2.01 (m, 1H), 1.60-1.51 (m, 4H). MS (ESI) m/z=374.1 [M+H]⁺.

### Example 5: 6-((2-(2,6-Dioxopiperidin-3-yl)-1,3-dioxoisoindolin-4-yl)amino)hexanoic acid (Handle 5)

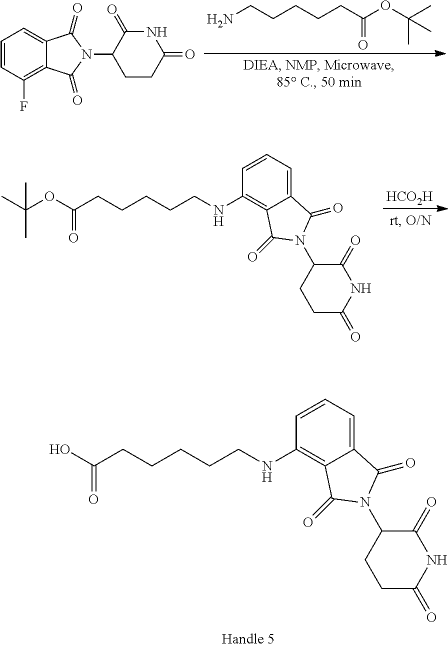 [SMILES: O=C1CCC(N2C(=O)c3cccc(F)c3C2=O)C(=O)N1 ; CC(C)(C)OC(=O)CCCCCN ; CC(C)(C)OC(=O)CCCCCNc1cccc2c1C(=O)N(C1CCC(=O)NC1=O)C2=O ; "HCO2H" ; O=C(O)CCCCCNc1cccc2c1C(=O)N(C1CCC(=O)NC1=O)C2=O]

Handle 5 was synthesized following the same procedures as Handle 1 as described in Example 1 (1.43 g, yield: 18% over 2 steps). ¹H NMR (400 MHz, DMSO-d₆) δ 11.97 (s, 1H), 11.08 (s, 1H), 7.57 (dd, J=7.2, 8.8 Hz, 1H), 7.08 (d, J=8.8 Hz, 1H), 7.02 (d, J=7.2 Hz, 1H), 6.52 (t, J=6.0 Hz, 1H), 5.05 (dd, J=5.6, 12.8 Hz, 1H), 3.30 (q, J=6.8 Hz, 2H), 2.93-2.83 (m, 1H), 2.61-2.50 (m, 2H), 2.32 (t, J=7.2 Hz, 2H), 2.07-2.00 (m, 1H), 1.61-1.50 (m, 4H), 1.39-1.33 (m, 2H). MS (ESI) m/z=388.1 [M+H]⁺.

### Example 6: 7-((2-(2,6-Dioxopiperidin-3-yl)-1,3-dioxoisoindolin-4-yl)amino)heptanoic acid (Handle 6)

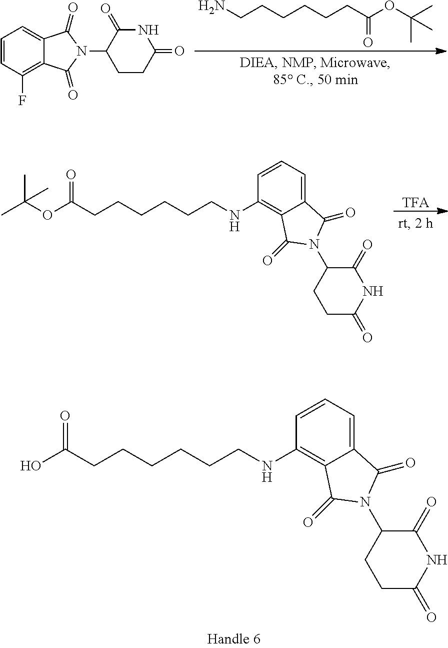 [SMILES: O=C1CCC(N2C(=O)c3cccc(F)c3C2=O)C(=O)N1 ; CC(C)(C)OC(=O)CCCCCCN ; CC(C)(C)OC(=O)CCCCCCNc1cccc2c1C(=O)N(C1CCC(=O)NC1=O)C2=O ; O=C(O)CCCCCCNc1cccc2c1C(=O)N(C1CCC(=O)NC1=O)C2=O ; "TFA"]

Handle 6 was synthesized following the same procedures as Handle 1 as described in Example 1 (2.3 g, yield: 24% over 2 steps). ¹H NMR (400 MHz, DMSO-d₆) δ 11.92 (brs, 1H), 11.08 (s, 1H), 7.57 (t, J=8.0 Hz, 1H), 7.13 (d, J=8.8 Hz, 1H), 7.03 (d, J=6.8 Hz, 1H), 6.52 (t, J=5.6 Hz, 1H), 5.05 (dd, J=5.6, 12.8 Hz, 1H), 3.30 (q, J=6.4 Hz, 2H), 2.93-2.83 (m, 1H), 2.61-2.50 (m, 2H), 2.31 (t, J=7.2 Hz, 2H), 2.07-2.00 (m, 1H), 1.58-1.48 (m, 4H), 1.34-1.31 (m, 4H). MS (ESI) m/z=402.1 [M+H]⁺.

### Example 7: 8-((2-(2,6-Dioxopiperidin-3-yl)-1,3-dioxoisoindolin-4-yl)amino)octanoic acid (Handle 7)

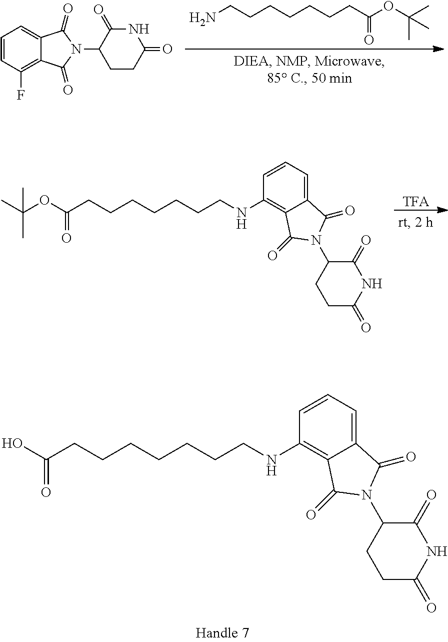 [SMILES: O=C1CCC(N2C(=O)c3cccc(F)c3C2=O)C(=O)N1 ; CC(C)(C)OC(=O)CCCCCCCN ; CC(C)(C)OC(=O)CCCCCCCNc1cccc2c1C(=O)N(C1CCC(=O)NC1=O)C2=O ; O=C(O)CCCCCCCNc1cccc2c1C(=O)N(C1CCC(=O)NC1=O)C2=O ; "TFA"]

Handle 7 was synthesized following the same procedures as Handle 1 as described in Example 1 (1.14 g, yield: 35% over 2 steps). ¹H NMR (400 MHz, DMSO-d₆) δ 11.94 (s, 1H), 11.08 (s, 1H), 7.57 (t, J=8.0 Hz, 1H), 7.08 (d, J=8.4 Hz, 1H), 7.02 (d, J=6.8 Hz, 1H), 6.52 (t, J=5.6 Hz, 1H), 5.05 (dd, J=5.6, 12.8 Hz, 1H), 3.31-3.26 (m, 2H), 2.93-2.83 (m, 1H), 2.61-2.50 (m, 2H), 2.19 (t, J=7.2 Hz, 2H), 2.05-2.00 (m, 1H), 1.58-1.47 (m, 4H), 1.35-1.25 (s, 6H). MS (ESI) m/z=416.1 [M+H]⁺.

### Example 8: 3-(2-((2-(2,6-Dioxopiperidin-3-yl)-1,3-dioxoisoindolin-4-yl)amino)ethoxy) propanoic acid (Handle 8)

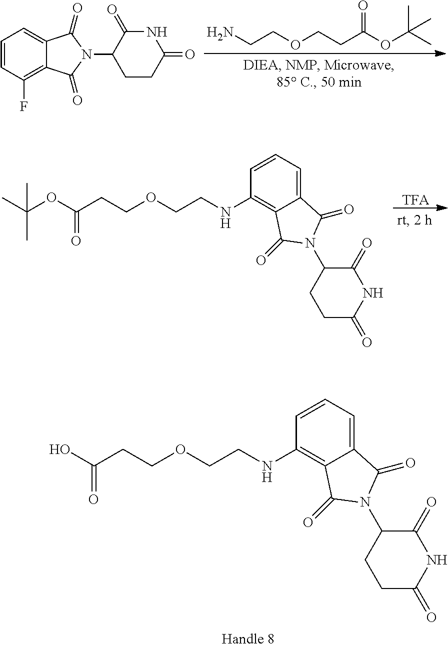 [SMILES: O=C1CCC(N2C(=O)c3cccc(F)c3C2=O)C(=O)N1 ; CC(C)(C)OC(=O)CCOCCN ; CC(C)(C)OC(=O)CCOCCNc1cccc2c1C(=O)N(C1CCC(=O)NC1=O)C2=O ; O=C(O)CCOCCNc1cccc2c1C(=O)N(C1CCC(=O)NC1=O)C2=O ; "TFA"]

Handle 8 was synthesized following the same procedures as Handle 1 as described in Example 1 (3.5 g, yield: 18% over 2 steps). ¹H NMR (400 MHz, DMSO-d₆) δ 12.18 (s, 1H), 11.08 (s, 1H), 7.58 (dd, J=7.2 Hz, 8.8 Hz, 1H), 7.13 (d, J=8.4 Hz, 1H), 7.04 (d, J=7.2 Hz, 1H), 6.58 (t, J=5.6 Hz, 1H), 5.05 (dd, J=6.4 Hz, 12.8 Hz, 1H), 3.67-3.58 (m, 4H), 3.47-3.43 (m, 2H), 2.93-2.84 (m, 1H), 2.61-2.45 (m, 4H), 2.07-2.01 (m, 1H). MS (ESI) m/z=390.1 [M+H]⁺.

### Example 9: 3-(2-(2-((2-(2,6-Dioxopiperidin-3-yl)-1,3-dioxoisoindolin-4-yl)amino)ethoxy)ethoxy)propanoic acid (Handle 9)

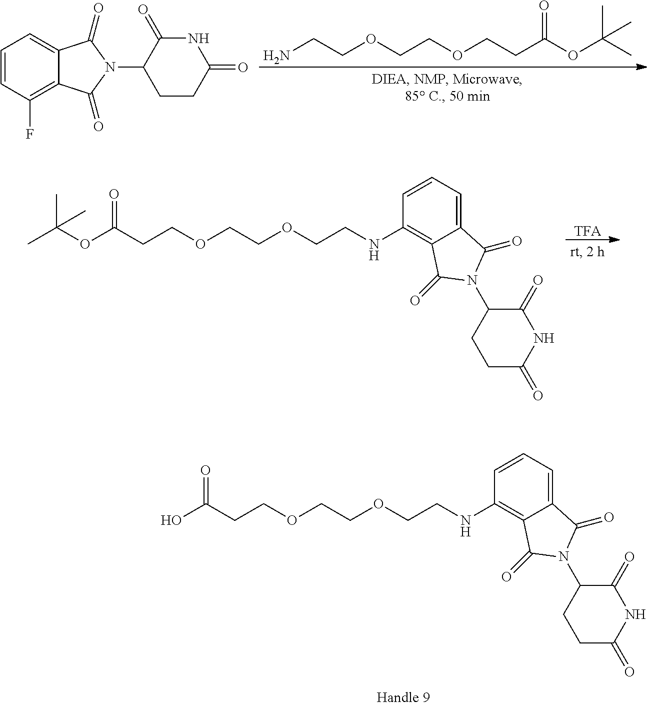

Handle 9 was synthesized following the same procedures as Handle 1 as described in Example 1 (2.0 g, yield: 24% over 2 steps). ¹H NMR (400 MHz, DMSO-d₆) δ 12.13 (s, 1H), 11.08 (s, 1H), 7.58 (dd, J=7.2 Hz, 8.4 Hz, 1H), 7.14 (d, J=8.4 Hz, 1H), 7.04 (d, J=6.8 Hz, 1H), 6.60 (t, J=6.0 Hz, 1H), 5.05 (dd, J=5.2 Hz, 12.4 Hz, 1H), 3.63-3.44 (m, 10H), 2.88-2.85 (m, 1H), 2.61-2.49 (m, 2H), 2.44-2.41 (m, 2H), 2.04-2.01 (m, 1H). MS (ESI) m/z=434.1 [M+H]⁺.

### Example 10: 3-(2-(2-(2-((2-(2,6-Dioxopiperidin-3-yl)-1,3-dioxoisoindolin-4-yl)amino)ethoxy)ethoxy)ethoxy)propanoic acid (Handle 10)

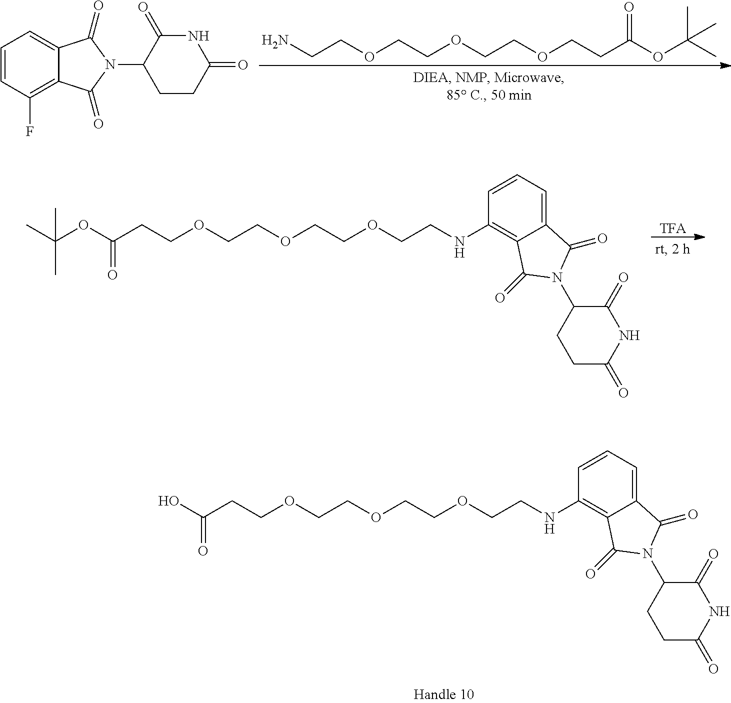

Handle 10 was synthesized following the same procedures as Handle 1 as described in Example 1 (3.2 g, yield: 42% over 2 steps). ¹H NMR (400 MHz, DMSO-d₆) δ 12.14 (s, 1H), 11.08 (s, 1H), 7.58 (dd, J=7.2 Hz, 8.4 Hz, 1H), 7.14 (d, J=8.8 Hz, 1H), 7.04 (d, J=6.8 Hz, 1H), 6.60 (t, J=6.0 Hz, 1H), 5.05 (dd, J=5.2 Hz, 12.8 Hz, 1H), 3.63-3.45 (m, 14H), 2.88-2.85 (m, 1H), 2.61-2.49 (m, 2H), 2.44-2.40 (m, 2H), 2.04-2.01 (m, 1H). MS (ESI) m/z=478.2 [M+H]⁺.

### Example 11: 1-((2-(2,6-Dioxopiperidin-3-yl)-1,3-dioxoisoindolin-4-yl)amino)-3,6,9,12-tetraoxapentadecan-15-oic acid (Handle 11)

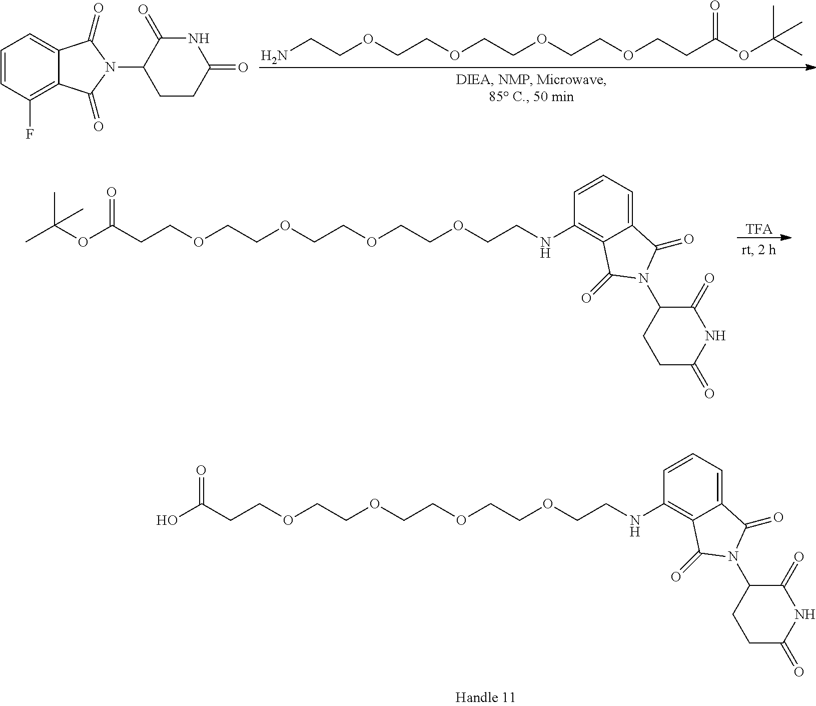

Handle 11 was synthesized following the same procedures as Handle 1 as described in Example 1 (2.3 g, yield: 31% over 2 steps). ¹H NMR (400 MHz, DMSO-d₆) δ 12.14 (s, 1H), 11.08 (s, 1H), 7.58 (dd, J=7.2 Hz, 8.8 Hz, 1H), 7.14 (d, J=8.4 Hz, 1H), 7.04 (d, J=7.2 Hz, 1H), 6.60 (t, J=6.0 Hz, 1H), 5.05 (dd, J=5.2 Hz, 12.8 Hz, 1H), 3.63-3.48 (m, 18H), 2.89-2.85 (m, 1H), 2.61-2.49 (m, 2H), 2.44-2.41 (m, 2H), 2.04-2.01 (m, 1H). MS (ESI) m/z=522.2 [M+H]⁺.

### Example 12: 1-((2-(2,6-Dioxopiperidin-3-yl)-1,3-dioxoisoindolin-4-yl)amino)-3,6,9,12,15-pentaoxaoctadecan-18-oic acid (Handle 12)

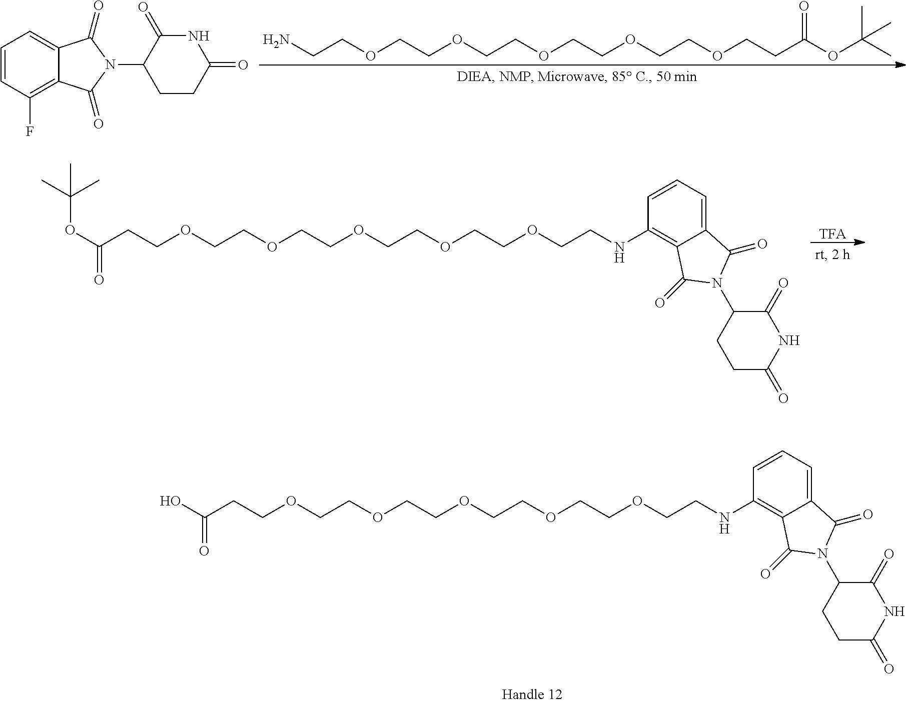

Handle 12 was synthesized following the same procedures as Handle 1 as described in Example 1 (2.4 g, yield: 36% over 2 steps). ¹H NMR (400 MHz, DMSO-d₆) δ 11.09 (s, 1H), 7.58 (dd, J=7.2, 8.4 Hz, 1H), 7.13 (d, J=8.4 Hz, 1H), 7.04 (d, J=7.2 Hz, 1H), 6.60 (t, J=5.6 Hz, 1H), 5.05 (dd, J=5.6, 12.8 Hz, 1H), 3.64-3.46 (m, 22H), 2.93-2.83 (m, 1H), 2.61-2.50 (m, 2H), 2.44-2.40 (m, 2H), 2.02 (t, J=6.4 Hz, 1H). MS (ESI) m/z=566.2 [M+H]⁺.

### Example 13: 4-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-4-oxobutanoic acid (Handle 13)

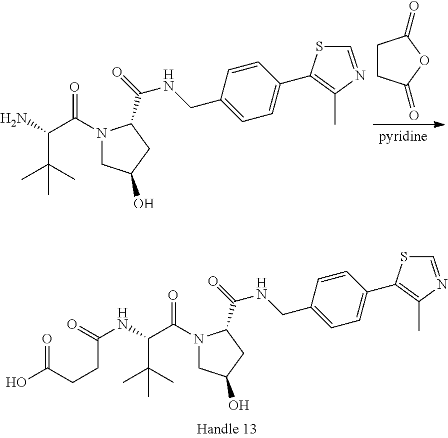

A mixture of (2S,4R)-1-((S)-2-amino-3,3-dimethylbutanoyl)-4-hydroxy-N-(4-(4-methylthiazol-5-yl)benzyl)pyrrolidine-2-carboxamide (1.0 g, 2.3 mmol) and succinic anhydride (465 mg, 4.65 mmol) in pyridine (5 mL) was stirred at rt overnight. The mixture was concentrated. The residue was purified by reverse-phase flash chromatography (MeCN/H₂O) to give the title compound (1.05 g, yield: 86%). ¹H NMR (400 MHz, DMSO-d₆) δ 12.02 (s, 1H), 8.99 (s, 1H), 8.58 (t, J=6.0 Hz, 1H), 7.96 (d, J=9.2 Hz, 1H), 7.43-7.37 (m, 4H), 5.13 (d, J=3.6 Hz, 1H), 4.53 (d, J=9.2 Hz, 1H), 4.46-4.40 (m, 2H), 4.34 (s, 1H), 4.21 (dd, J=16.0, 5.2 Hz, 1H), 3.69-3.60 (m, 2H), 2.45 (s, 3H), 2.44-2.33 (m, 4H), 2.06-2.01 (m, 1H), 1.93-1.87 (m, 1H), 0.93 (s, 9H). ¹³C NMR (100 MHz, DMSO-d₆): δ 173.83, 171.92, 170.86, 169.56, 151.41, 147.70, 139.48, 131.15, 129.63, 128.62, 127.41, 68.87, 58.70, 56.44, 56.34, 41.65, 37.91, 35.35, 29.74, 29.25, 26.35, 15.92. MS (ESI) m/z: 531.2 [M+H]⁺.

### Example 14: 5-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-5-oxopentanoic acid (Handle 14)

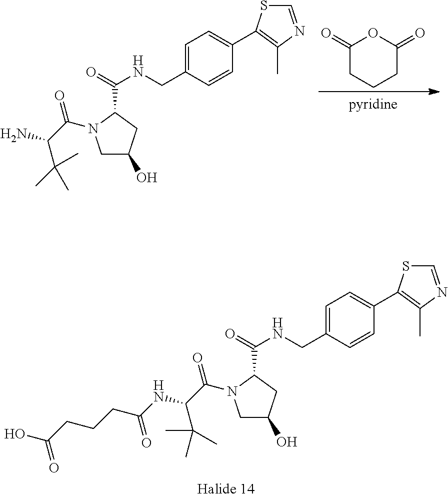

Handle 14 was synthesized following the same procedures as Handle 13 as described in Example 13 (1.5 g, yield: 79%). ¹H NMR (400 MHz, DMSO-d₆) δ 8.99 (s, 1H), 8.59 (t, J=6.0 Hz, 1H), 7.91 (d, J=9.2 Hz, 1H), 7.44-7.37 (m, 4H), 5.16 (brs, 1H), 4.54 (d, J=9.2 Hz, 1H), 4.47-4.42 (m, 2H), 4.36 (s, 1H), 4.21 (dd, J=16.0, 5.2 Hz, 1H), 3.7-3.64 (m, 2H), 2.45 (s, 3H), 2.31-2.14 (m, 4H), 2.07-2.02 (m, 1H), 1.94-1.81 (m, 1H), 1.74-1.68 (m, 2H), 0.94 (s, 9H). ¹³C NMR (100 MHz, DMSO-d₆): δ 174.18, 171.94, 171.63, 169.66, 151.41, 147.70, 139.46, 131.15, 129.61, 128.62, 127.41, 68.86, 58.69, 56.38, 41.65, 37.91, 35.16, 34.03, 33.10, 26.35, 20.89, 15.92. MS (ESI) m/z=543.2 [M−H]⁻.

### Example 15: 6-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-6-oxohexanoic acid (Handle 15)

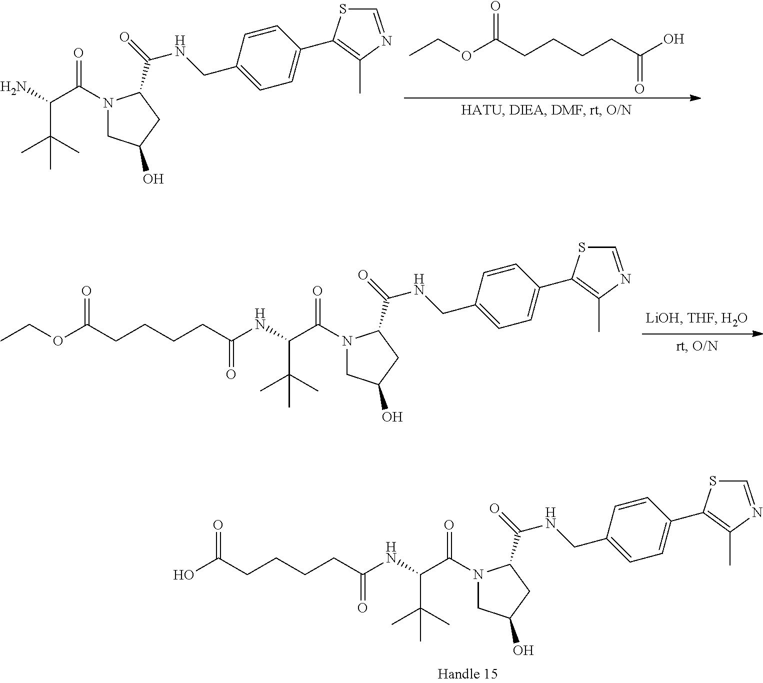

### Step 1. Synthesis of ethyl 6-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-6-oxohexanoate

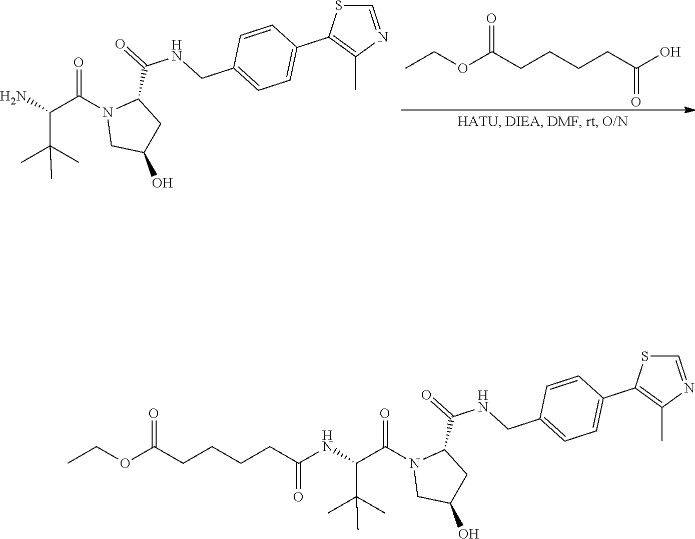

To a solution of (2S,4R)-1-((S)-2-amino-3,3-dimethylbutanoyl)-4-hydroxy-N-(4-(4-methylthiazol-5-yl)benzyl)pyrrolidine-2-carboxamide (2.00 g, 4.65 mmol), 6-ethoxy-6-oxohexanoic acid (809 mg, 4.65 mmol) in DMF (20 mL) was added DIEA (3.03 g, 23.26 mmol) and HATU (3.53 g, 9.30 mmol) at rt. After the mixture was stirred at rt overnight, it was diluted with H₂O (100 mL) and extracted with EtOAc (3×100 mL). The combined organic layers were washed with brine (2×100 mL), dried over anhydrous sodium sulfate, filtered, concentrated and purified by flash chromatography on silica gel (DCM/MeOH=20:1) to give the title compound (1.70 g, yield: 74%) as a yellow solid. MS (ESI) m/z=587.3 [M+H]⁺.

### Step 2. Synthesis of 6-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-6-oxohexanoic acid

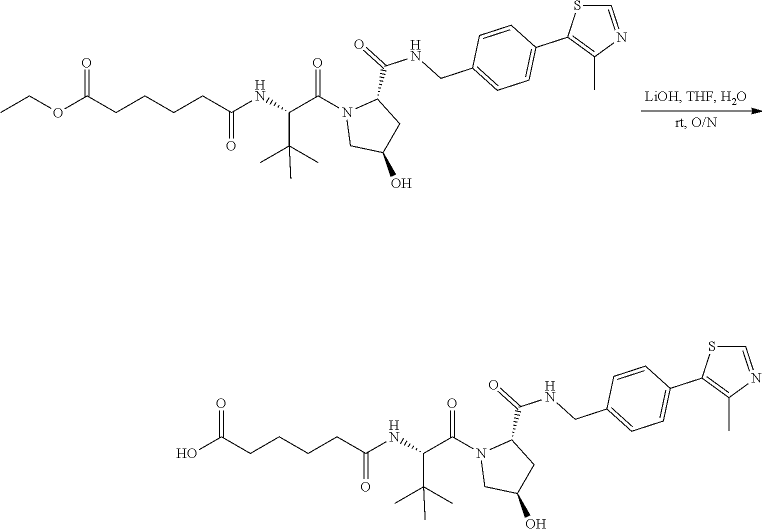 [SMILES: CCOC(=O)CCCCC(=O)N[C@H](C(=O)N1C[C@H](O)C[C@H]1C(=O)NCc1ccc(-c2scnc2C)cc1)C(C)(C)C ; Cc1ncsc1-c1ccc(CNC(=O)[C@@H]2C[C@@H](O)CN2C(=O)[C@@H](NC(=O)CCCCC(=O)O)C(C)(C)C)cc1]

To a solution of ethyl 6-(((S)-1-((2S,4R)-4-hydroxy-2-((4-(4-methylthiazol-5-yl)benzyl) carbamoyl)pyrrolidin-1-yl)-3,3-dimethyl-1-oxobutan-2-yl)amino)-6-oxohexanoate (1.70 g, 3.50 mmol) in THF (20 mL) and H₂O (20 mL) was added LiOH·H₂O (294 mg, 7.00 mmol) at rt. The mixture was stirred at rt overnight. THF was removed under reduced pressure and the residue was pH was adjusted to 6 with hydrochloric acid (1N). The precipitate was collected to give the title compound (1.198 g, yield: 74%) as a white solid. ¹H NMR (400 MHz, CDCl₃) δ 8.68 (s, 1H), 7.75 (s, 1H), 7.32-7.27 (m, 5H), 4.64-4.57 (m, 3H), 4.56-4.50 (m, 1H), 4.28-4.25 (m, 1H), 4.02-3.99 (m, 1H), 3.71-3.68 (m, 1H), 2.47 (s, 3H), 2.24-2.18 (m, 6H), 1.59-1.48 (m, 4H), 0.96 (s, 9H). MS (ESI) m/z=559.3 [M+H]⁺.

### Example 16: 7-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-7-oxoheptanoic acid (Handle 16)

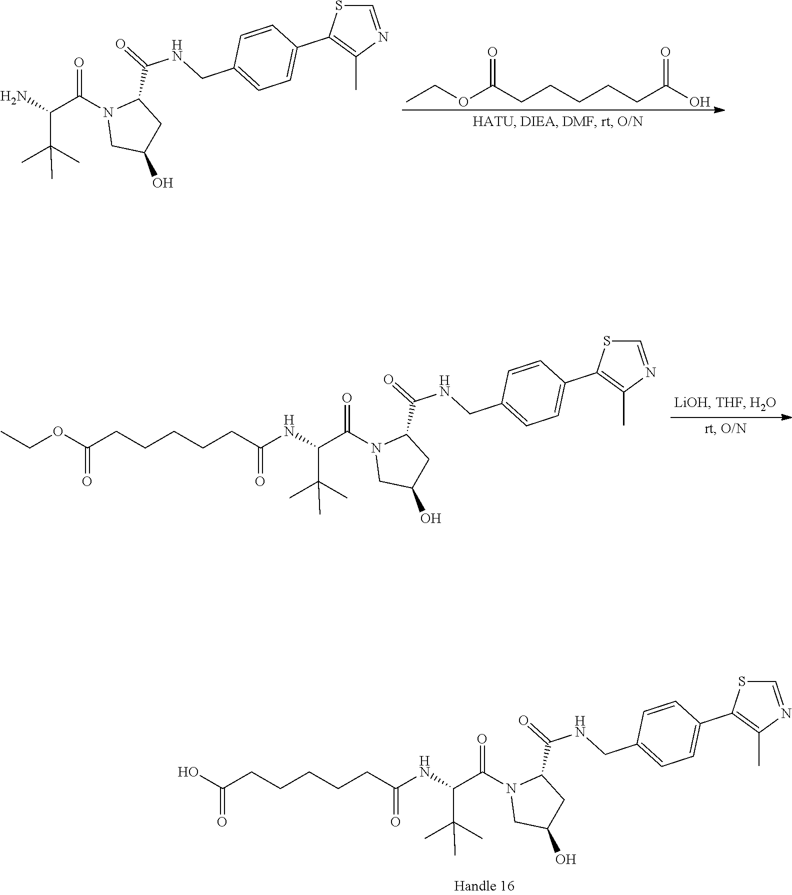 [SMILES: Cc1ncsc1-c1ccc(CNC(=O)[C@@H]2C[C@@H](O)CN2C(=O)[C@@H](N)C(C)(C)C)cc1 ; CCOC(=O)CCCCCC(=O)O ; CCOC(=O)CCCCCC(=O)N[C@H](C(=O)N1C[C@H](O)C[C@H]1C(=O)NCc1ccc(-c2scnc2C)cc1)C(C)(C)C ; Cc1ncsc1-c1ccc(CNC(=O)[C@@H]2C[C@@H](O)CN2C(=O)[C@@H](NC(=O)CCCCCC(=O)O)C(C)(C)C)cc1]

Handle 16 was synthesized following the same procedures as Handle 15 as described in Example 15 (1.1 g, yield: 33% over 2 steps). ¹H NMR (400 MHz, CDCl₃) δ 8.67 (s, 1H), 7.56-7.55 (m, 1H), 7.34-7.30 (m, 5H), 4.68-4.59 (m, 3H), 4.59-4.51 (m, 1H), 4.25 (dd, J=4.8 Hz, 15.2 Hz, 1H), 4.06-4.03 (m, 1H), 3.70-3.68 (m, 1H), 2.46 (s, 3H), 2.31-2.11 (m, 6H), 1.55-1.51 (m, 4H), 1.29-1.24 (m, 2H), 0.94 (s, 9H). MS (ESI) m/z=573.1 [M+H]⁺.

### Example 17: 8-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-8-oxooctanoic acid (Handle 17)

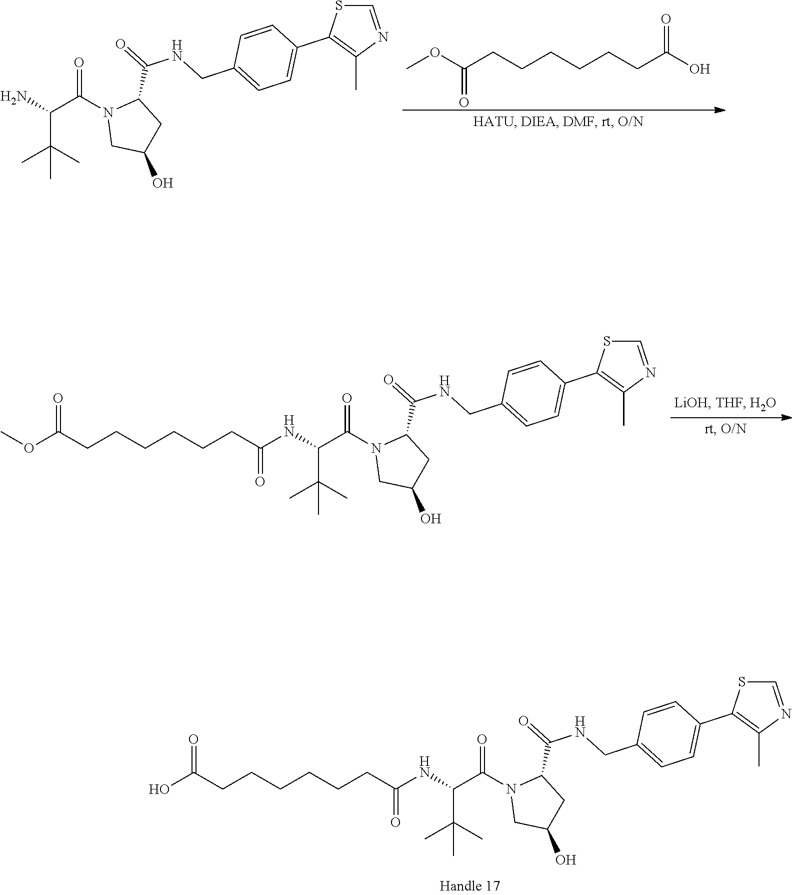

Handle 17 was synthesized following the same procedures as Handle 15 as described in Example 15 (1.08 g, yield: 52% over 2 steps). ¹H NMR (400 MHz, DMSO-d₆) δ 8.99 (s, 1H), 8.55 (t, J=2.4 Hz, 1H), 7.83 (d, J=9.2 Hz, 1H), 7.44-7.38 (m, 4H), 4.55 (d, J=9.6 Hz, 1H), 4.52-4.41 (m, 2H), 4.36 (s, 1H), 4.25-4.21 (m, 1H), 3.67-3.66 (m, 2H), 2.45 (s, 3H), 2.30-1.91 (m, 6H), 1.49-1.47 (m, 4H), 1.26-1.24 (m, 4H), 0.92 (s, 9H). MS (ESI) m/z=587.3 [M+H]⁺.

### Example 18: 9-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-9-oxononanoic acid (Handle 18)

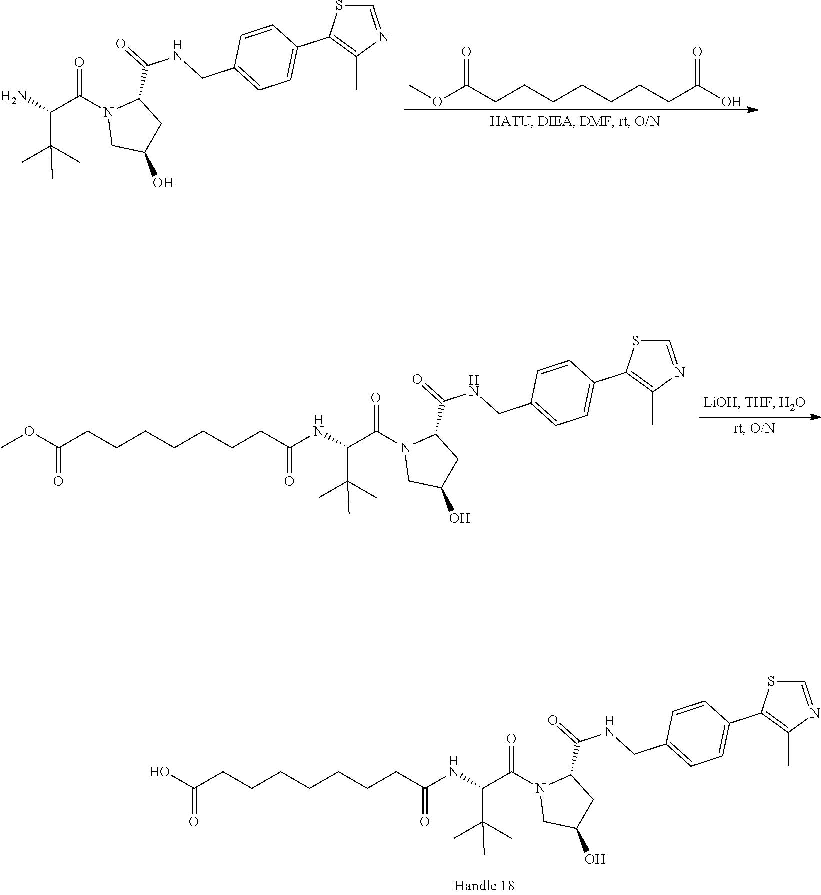 [SMILES: Cc1ncsc1-c1ccc(CNC(=O)[C@@H]2C[C@@H](O)CN2C(=O)[C@@H](N)C(C)(C)C)cc1 ; COC(=O)CCCCCCCC(=O)O ; COC(=O)CCCCCCCC(=O)N[C@H](C(=O)N1C[C@H](O)C[C@H]1C(=O)NCc1ccc(-c2scnc2C)cc1)C(C)(C)C ; Cc1ncsc1-c1ccc(CNC(=O)[C@@H]2C[C@@H](O)CN2C(=O)[C@@H](NC(=O)CCCCCCCC(=O)O)C(C)(C)C)cc1]

Handle 18 was synthesized following the same procedures as Handle 15 as described in Example 15 (1.16 g, yield: 44% over 2 steps). ¹H NMR (400 MHz, CDCl₃) δ 8.70 (s, 1H), 7.55 (s, 1H), 7.33-7.27 (m, 4H), 7.08 (d, J=8.0 Hz, 1H), 4.68-4.52 (m, 4H), 4.31-4.27 (m, 1H), 4.08-4.05 (m, 1H), 3.69-3.67 (m, 1H), 2.48 (s, 3H), 2.33-2.11 (m, 6H), 1.60-1.47 (m, 4H), 1.29-1.20 (m, 6H), 0.96 (s, 9H). MS (ESI) m/z=601.1 [M+H]⁺.

### Example 19: 10-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-10-oxodecanoic acid (Handle 19)

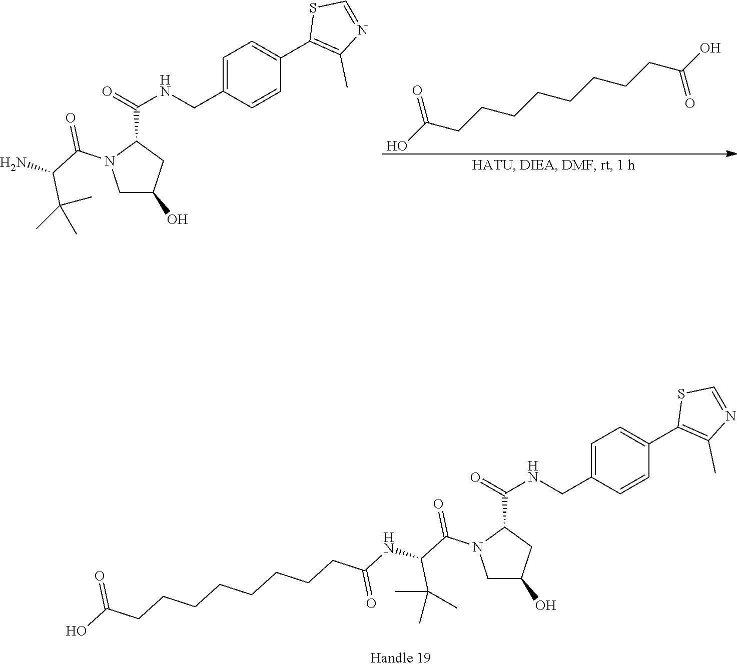 [SMILES: Cc1ncsc1-c1ccc(CNC(=O)[C@@H]2C[C@@H](O)CN2C(=O)[C@@H](N)C(C)(C)C)cc1 ; O=C(O)CCCCCCCCC(=O)O ; Cc1ncsc1-c1ccc(CNC(=O)[C@@H]2C[C@@H](O)CN2C(=O)[C@@H](NC(=O)CCCCCCCCC(=O)O)C(C)(C)C)cc1]

Handle 19 was synthesized following the same procedure as Handle 15 as described in Example 45 (1.1 g, yield: 35%). ¹H NMR (400 MHz, DMSO-d₆) δ 8.99 (s, 1H), 8.58 (t, J=6.0 Hz, 1H), 7.85 (d, J=9.2 Hz, 1H), 7.43-7.37 (m, 4H), 4.54 (d, J=9.2 Hz, 1H), 4.47-4.41 (m, 2H), 4.35 (s, 1H), 4.21 (dd, J=16.0, 5.6 Hz, 1H), 3.69-3.63 (m, 2H), 2.45 (s, 3H), 2.29-2.09 (m, 4H), 2.03-2.01 (m, 1H), 1.94-1.88 (m, 1H), 1.47 (m, 4H), 1.24 (br, 8H), 0.94 (s, 9H). ¹³C NMR (100 MHz, DMSO-d₆): δ 172.07, 171.92, 169.69, 151.41, 147.70, 139.48, 131.14, 129.62, 128.61, 127.40, 68.84, 58.67, 56.32, 56.26, 41.64, 37.93, 35.18, 34.85, 28.62, 26.36, 25.39, 15.93. MS (ESI) m/z=615.3 [M+H]⁺.

### Example 20: 11-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-11-oxoundecanoic acid (Handle 20)

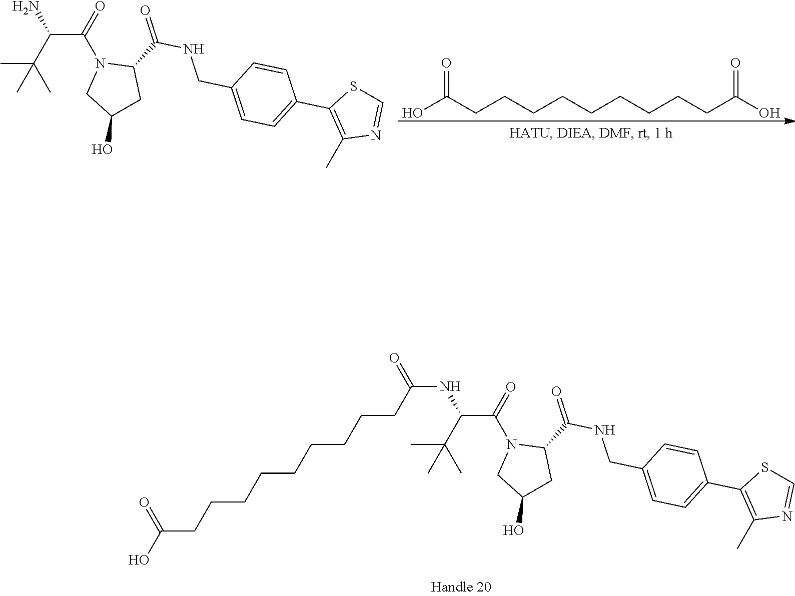

Handle 20 was synthesized following the same procedure as Handle 15 as described in Example 15 (1.1 g, yield: 50%). ¹H NMR (400 MHz, DMSO-d₆) δ 8.99 (s, 1H), 8.58 (t, J=6.0 Hz, 1H), 7.85 (t, J=9.2 Hz, 1H), 7.37-7.43 (m, 4H), 4.56-4.19 (m, 5H), 3.70-3.60 (m, 2H), 2.45 (s, 3H), 2.27-1.90 (m, 6H), 1.49-1.45 (m, 4H), 1.23 (m, 10H), 0.93 (s, 9H). ¹³C NMR (100 MHz, DMSO-d₆): δ 174.59, 172.07, 171.92, 169.69, 151.42, 147.70, 139.49, 131.14, 129.62, 128.61, 127.41, 68.84, 58.67, 56.32, 56.25, 41.64, 37.93, 35.19, 34.85, 33.80, 28.82, 28.70, 28.68, 28.62, 28.55, 26.37, 25.42, 24.55, 15.93. MS (ESI) m/z=629.4 [M+H]⁺.

### Example 21: 3-(3-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-3-oxopropoxy)propanoic acid (Handle 21)

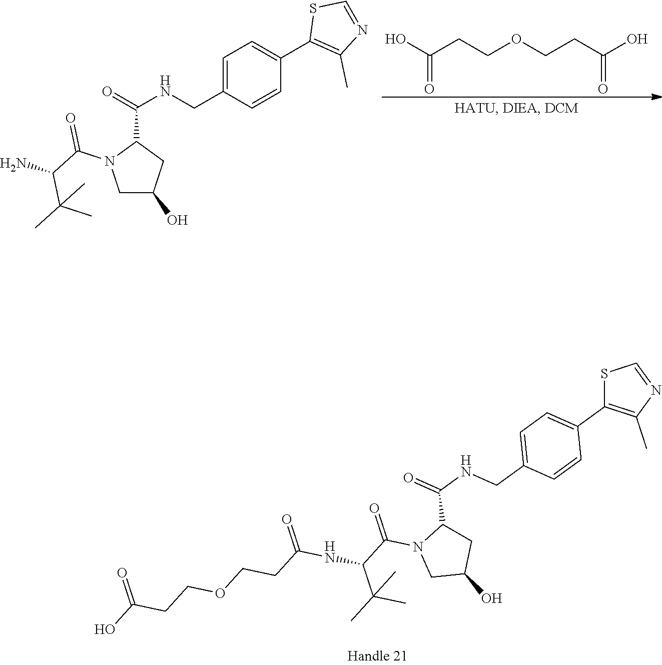

Handle 21 was synthesized following the same procedure as Handle 15 as described in Example 15 (1.1 g, yield: 42%). ¹H NMR (400 MHz, DMSO-d₆) δ 8.98 (s, 1H), 8.55 (t, J=6.0 Hz, 1H), 7.91 (d, J=9.2 Hz, 1H), 7.43-7.37 (m, 4H), 4.55-4.53 (m, 1H), 4.45-4.40 (m, 2H), 4.35 (s, 1H), 4.24-4.19 (m, 1H), 3.68-3.52 (m, 6H), 2.54-2.56 (m, 1H), 2.45-2.37 (m, 5H), 2.34-2.30 (m, 1H), 2.05-2.00 (m, 1H), 1.93-1.86 (m, 1H), 0.93 (s, 9H). MS (ESI) m/z=575 [M+H]⁺.

### Example 22: 2-(2-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-2-oxoethoxy)acetic acid (Handle 22)

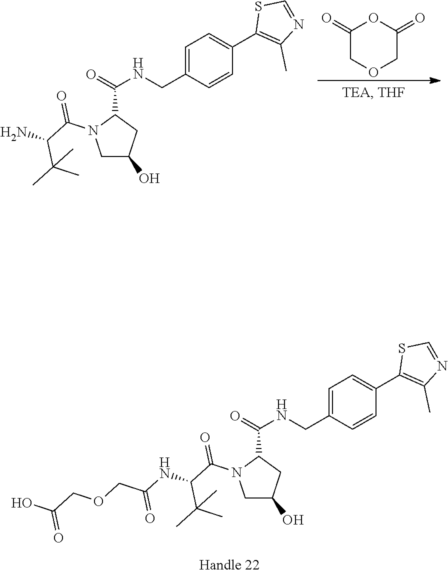

Handle 22 was synthesized following the same procedure as Handle 13 as described in Example 13 (1.2 g, yield: 63%). ¹H NMR (400 MHz, DMSO-d₆) δ 12.81 (brs, 1H), 8.98 (s, 1H), 8.58 (t, J=6.0 Hz, 1H), 7.60 (d, J=9.6 Hz, 1H), 7.45-7.35 (m, 4H), 5.14 (brs, 1H), 4.58-4.55 (m, 1H), 4.46-4.36 (m, 3H), 4.28-4.26 (m, 1H), 4.14 (s, 2H), 4.04 (s, 2H), 3.69-3.60 (m, 2H), 2.44 (s, 3H), 2.08-2.03 (m, 1H), 1.93-1.87 (m, 1H), 0.95 (s, 9H). MS (ESI) m/z=547 [M+H]⁺.

### Example 23: 3-(2-(3-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethoxy)propanoic acid (Handle 23)

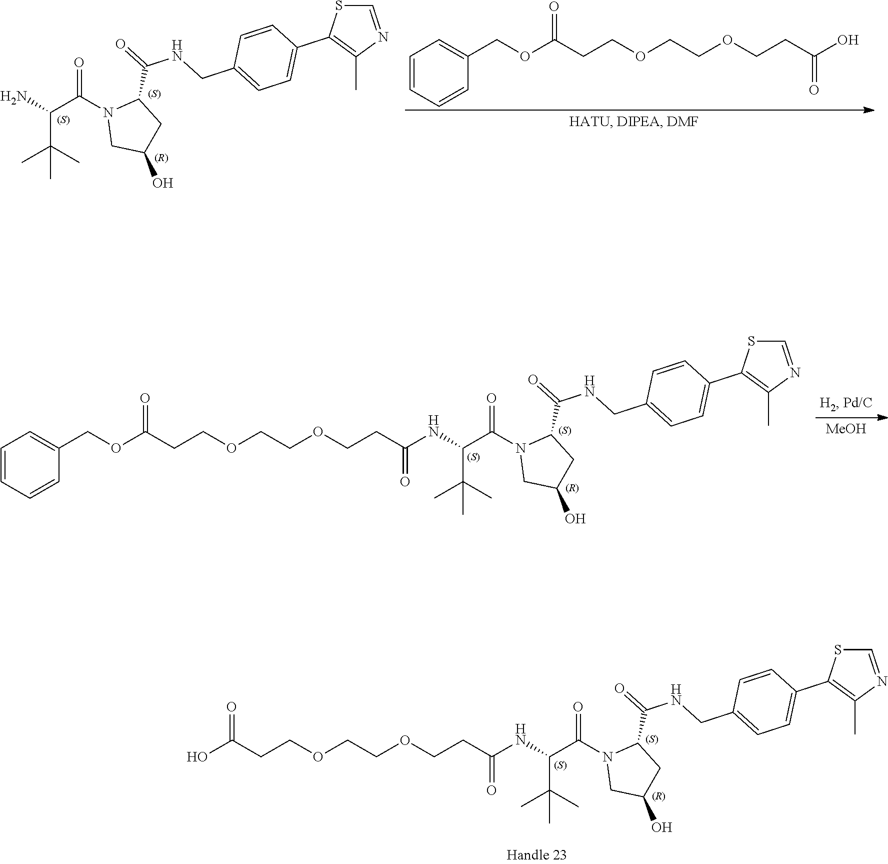

Handle 23 was synthesized following the same procedures as Handle 15 as described in Example 15 (1.4 g, yield 23% over 2 steps). ¹H NMR (400 MHz, DMSO-d₆) δ 8.98 (s, 1H), 8.56 (t, J=6.0 Hz, 1H), 7.91 (d, J=9.2 Hz, 1H), 7.43-7.37 (m, 4H), 4.55 (d, J=9.6 Hz, 1H), 4.46-4.41 (m, 2H), 4.35 (s, 1H), 4.29-4.20 (m, 1H), 3.70-3.57 (m, 7H), 3.50-3.45 (m, 5H), 2.57-2.55 (m, 1H), 2.45 (s, 3H), 2.43-2.41 (m, 1H), 2.37-2.32 (m, 1H), 2.09-2.01 (m, 1H), 1.94-1.87 (m, 1H), 0.94 (s, 9H). MS (ESI) m/z=619.3 [M+H]⁺.

### Example 24: 2-(2-(2-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethoxy)acetic acid (Handle 24)

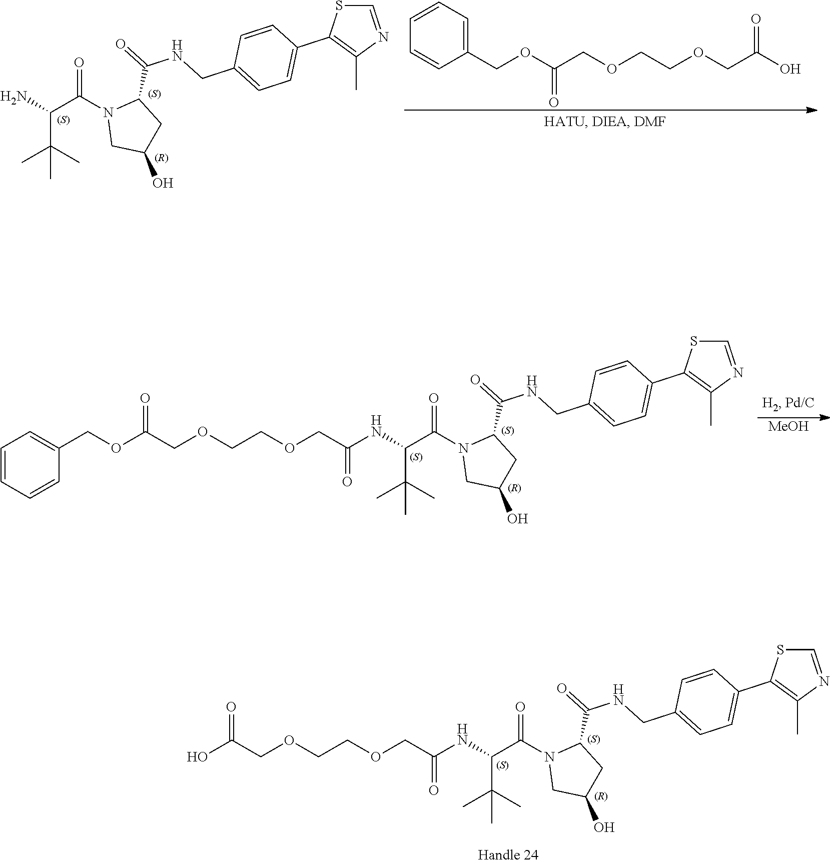

Handle 24 was synthesized following the same procedures as Handle 15 as described in Example 15 (1.13 g, yield 20% over 2 steps). ¹H NMR (400 MHz, DMSO-d₆) δ 8.98 (s, 1H), 8.60 (t, J=6.0 Hz, 1H), 7.49 (d, J=9.2 Hz, 1H), 7.40 (s, 4H), 4.57 (d, J=9.2 Hz, 1H), 4.47-4.36 (m, 3H), 4.28-4.23 (m, 1H), 4.05-3.93 (m, 4H), 3.69-3.61 (m, 6H), 2.45 (s, 3H), 2.08-2.03 (m, 1H), 1.94-1.87 (m, 1H), 0.94 (s, 9H). MS (ESI) m/z=591.2 [M+H]⁺.

### Example 25: (S)-15-((2S,4R)-4-Hydroxy-2-((4-(4-methylthiazol-5-yl)benzyl)carbamoyl)pyrrolidine-1-carbonyl)-16,16-dimethyl-13-oxo-4,7,10-trioxa-14-azaheptadecanoic acid (Handle 25)

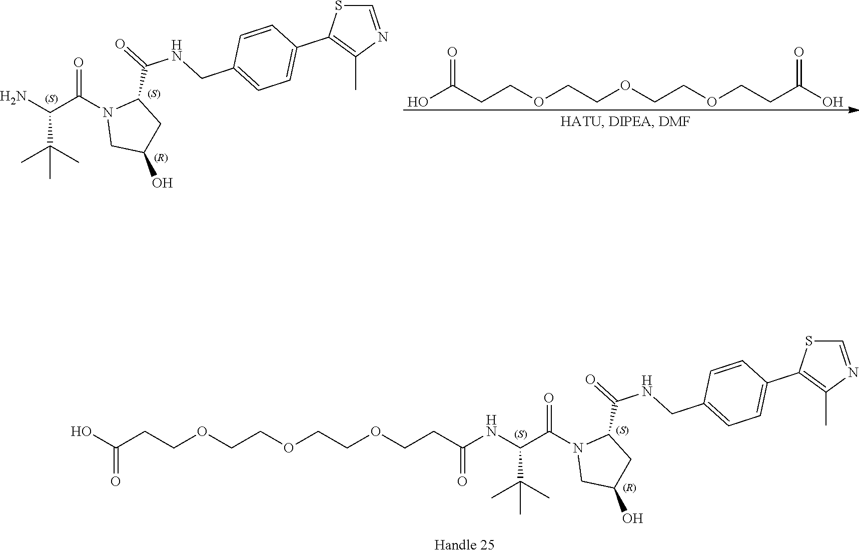

Handle 25 was synthesized following the same procedure as Handle 15 as described in Example 15 (1.7 g, yield 37%). ¹H NMR (400 MHz, DMSO-d₆) δ 8.99 (s, 1H), 8.56 (t, J=6.0 Hz, 1H), 7.91 (d, J=9.6 Hz, 1H), 7.44-7.38 (m, 4H), 4.56 (d, J=9.2 Hz, 1H), 4.47-4.42 (m, 2H), 4.36 (s, 1H), 4.25-4.20 (m, 1H), 3.70-3.55 (m, 6H), 3.50-3.46 (m, 8H), 2.58-2.51 (m, 3H), 2.45-2.42 (m, 5H), 2.40-2.33 (m, 1H), 2.07-2.02 (m, 1H), 1.94-1.88 (m, 1H), 0.94 (s, 9H). LCMS (ESI) m/z=661.0 [M−H]⁻.

### Example 26: (S)-13-((2S,4R)-4-Hydroxy-2-((4-(4-methylthiazol-5-yl)benzyl)carbamoyl)pyrrolidine-1-carbonyl)-14,14-dimethyl-11-oxo-3,6,9-trioxa-12-azapentadecanoic acid (Handle 26)

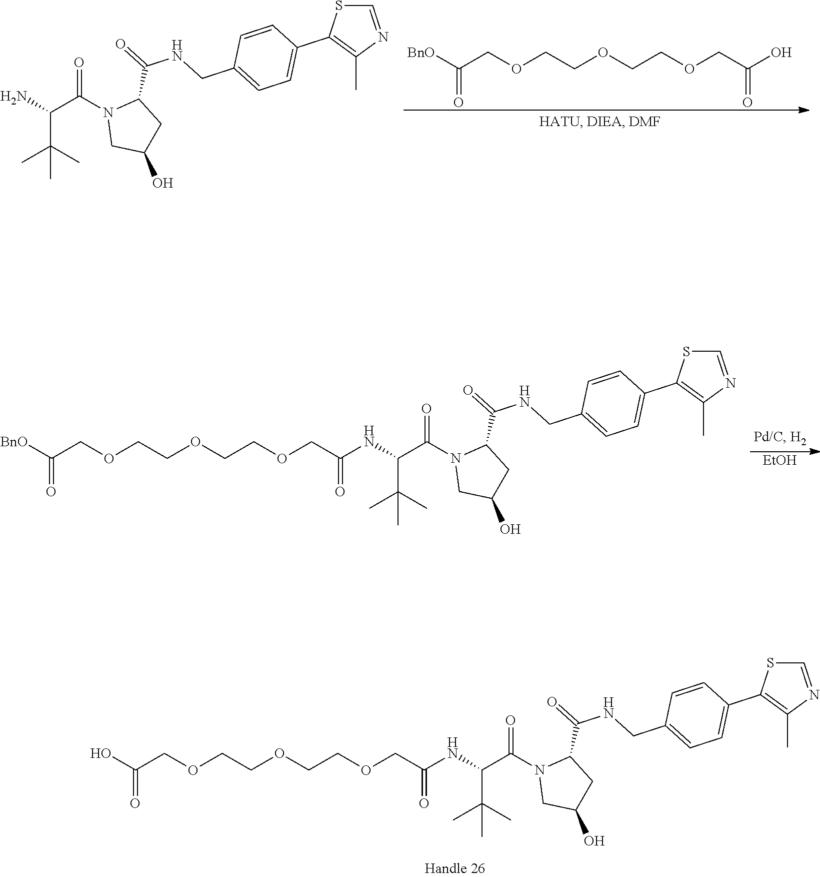

Handle 26 was synthesized following the same procedures as Handle 15 as described in Example 15 (1.21 g, yield 31% over 2 steps). ¹H NMR (400 MHz, CDCl₃) δ 8.68 (s, 1H), 7.80-7.71 (m, 11H), 7.41-7.33 (m, 5H), 4.71-7.65 (m, 1H), 4.61-4.50 (m, 3H), 4.37-4.33 (m, 1H), 4.07-3.94 (m, 5H), 3.77-3.58 (m, 10H), 2.51 (s, 3H), 2.38-2.30 (m, 1H), 2.24-2.19 (m, 1H), 0.98 (s, 9H). LCMS (ESI) m/z=635.0 [M+H]⁺.

### Example 27: (S)-18-((2S,4R)-4-Hydroxy-2-((4-(4-methylthiazol-5-yl)benzyl)carbamoyl)pyrrolidine-1-carbonyl)-19,19-dimethyl-16-oxo-4,7,10,13-tetraoxa-17-azaicosanoic acid (Handle 27)

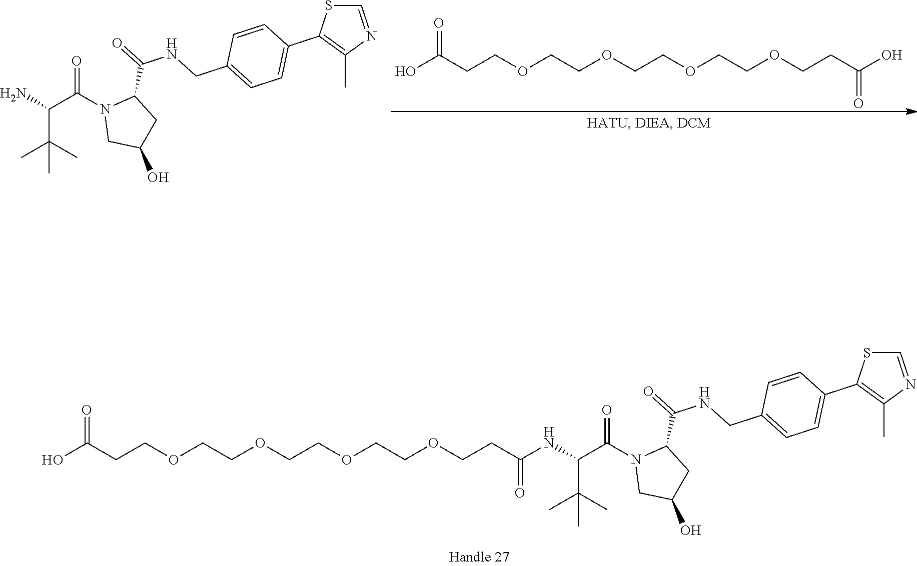 [SMILES: Cc1ncsc1-c1ccc(CNC(=O)[C@@H]2C[C@@H](O)CN2C(=O)[C@@H](N)C(C)(C)C)cc1 ; O=C(O)CCOCCOCCOCCOCCC(=O)O ; Cc1ncsc1-c1ccc(CNC(=O)[C@@H]2C[C@@H](O)CN2C(=O)[C@@H](NC(=O)CCOCCOCCOCCOCCC(=O)O)C(C)(C)C)cc1]

Handle 27 was synthesized following the same procedure as Handle 15 as described in Example 15 (1.6 g, yield 43%). ¹H NMR (400 MHz, CDCl₃) δ 8.69 (s, 1H), 7.55-7.52 (m, 1H), 7.47-7.45 (m, 1H), 7.36 (s, 4H), 4.70-4.66 (m, 1H), 4.62-4.57 (m, 2H), 4.50 (s, 1H), 4.34-4.29 (m, 1H), 4.12-4.09 (m, 1H), 3.75-3.48 (m, 18H), 2.56-2.47 (m, 7H), 2.40-2.33 (m, 1H), 2.23-2.18 (m, 1H), 0.96 (s, 9H). MS (ESI) m/z=707.1 [M+H]⁺.

### Example 28: (S)-21-((2S,4R)-4-Hydroxy-2-((4-(4-methylthiazol-5-yl)benzyl)carbamoyl)pyrrolidine-1-carbonyl)-22,22-dimethyl-19-oxo-4,7,10,13,16-pentaoxa-20-azatricosanoic acid (Handle 28)

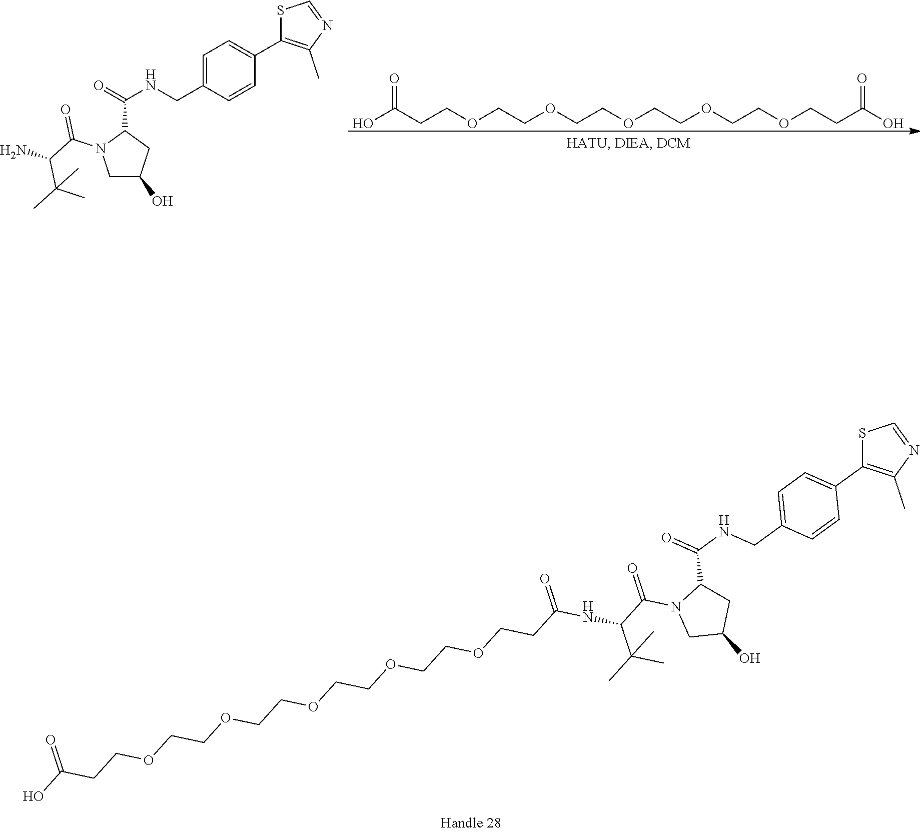

Handle 28 was synthesized following the same procedure as Handle 15 as described in Example 15 (1.2 g, yield: 23%). ¹H NMR (400 MHz, DMSO-d₆) δ 8.98 (s, 1H), 8.57 (t, J=6.0 Hz, 1H), 7.91 (d, J=9.6 Hz, 1H), 7.43-7.31 (m, 4H), 4.56-4.53 (m, 1H), 4.45-4.35 (m, 3H), 4.24-4.19 (m, 1H), 3.69-3.55 (m, 6H), 3.49-3.47 (m, 16H), 2.57-2.53 (m, 1H), 2.45 (s, 3H), 2.39-2.32 (m, 3H), 2.06-2.01 (m, 1H), 1.93-1.86 (m, 1H), 0.95 (s, 9H). MS (ESI) m/z=751 [M+H]⁺.

### Example 29: (S)-19-((2S,4R)-4-Hydroxy-2-((4-(4-methylthiazol-5-yl)benzyl)carbamoyl)pyrrolidine-1-carbonyl)-20,20-dimethyl-17-oxo-3,6,9,12,15-pentaoxa-18-azahenicosanoic acid (Handle 29)

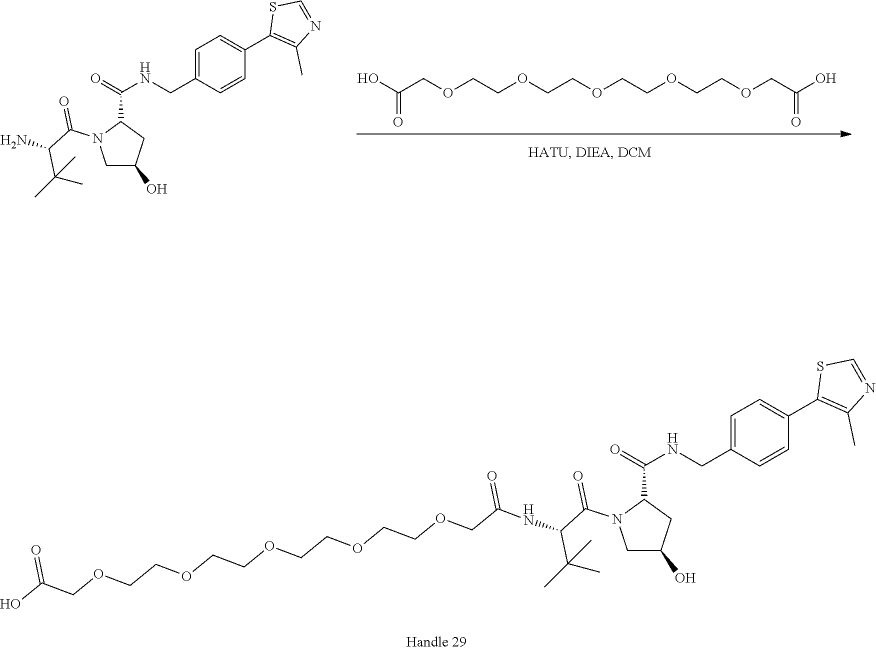

Handle 29 was synthesized following the same procedure as Handle 15 as described in Example 15 (1.3 g, yield: 39%). ¹H NMR (400 MHz, DMSO-d₆) δ 8.98 (s, 1H), 8.69 (t, J=6.0 Hz, 1H), 7.45 (d, J=9.6 Hz, 1H), 7.43-7.37 (m, 4H), 4.57-4.55 (m, 1H), 4.47-4.34 (m, 3H), 4.27-4.22 (m, 1H), 3.97 (s, 2H), 3.68-3.65 (m, 2H), 3.61-3.48 (m, 18H), 2.45 (s, 3H), 2.09-2.04 (m, 1H), 1.92-1.86 (m, 1H), 0.94 (s, 9H). MS (ESI) m/z=723 [M+H]⁺.

### Example 30: (2-(2,6-Dioxopiperidin-3-yl)-1,3-dioxoisoindolin-5-yl)glycine (Handle 30)

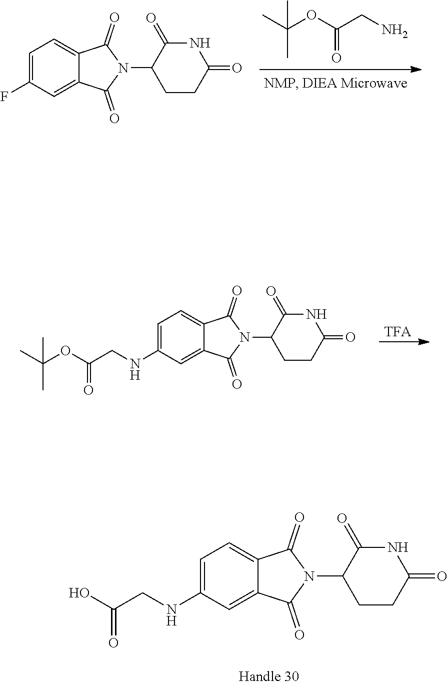

Handle 30 was synthesized following the same procedure as Handle 1 as described in Example 1 (1.0 g, yield: 84%). ¹H NMR (400 MHz, DMSO-d₆) δ 12.80 (brs, 1H), 11.06 (s, 1H), 7.59 (d, J=8.4 Hz, 1H), 7.32 (brs, 1H), 6.98 (d, J=1.2 Hz, 1H), 6.89 (dd, J=2.0, 8.4 Hz, 1H), 5.04 (dd, J=5.6, 13.2 Hz, 1H), 4.03 (s, 2H), 2.92-2.83 (m, 1H), 2.60-2.52 (m, 2H), 2.03-1.98 (m, 1H). MS (ESI) m/z=332.0 [M+H]⁺.

### Example 31: 3-((2-(2,6-Dioxopiperidin-3-yl)-1,3-dioxoisoindolin-5-yl)amino)propanoic acid (Handle 31)

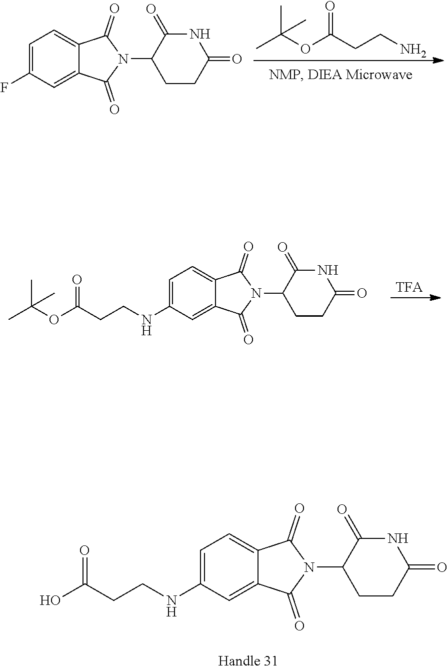

Handle 31 was synthesized following the same procedure as handle 1 as described in Example 1 (1.24 g, yield: 60%). [M+H]⁺. ¹H NMR (400 MHz, DMSO-d₆) δ 11.05 (s, 1H), 7.57 (d, J=8.4 Hz, 1H), 6.97 (d, J=2.0 Hz, 1H), 6.87 (dd, J=2.0, 8.4 Hz, 1H), 5.02 (dd, J=5.2, 12.8 Hz, 1H), 3.41 (t, J=6.8 Hz, 2H), 2.89-2.83 (m, 1H), 2.60-2.52 (m, 4H), 2.02-1.97 (m, 1H). MS (ESI) m/z=346.0

### Example 32: 4-((2-(2,6-Dioxopiperidin-3-yl)-1,3-dioxoisoindolin-5-yl)amino)butanoic acid (Handle 32)

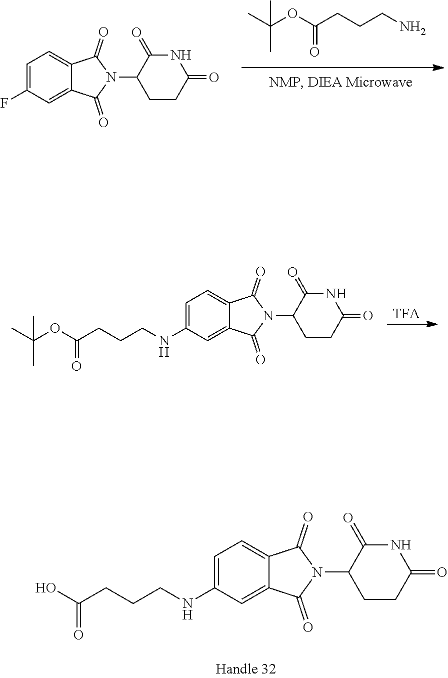 [SMILES: O=C1CCC(N2C(=O)c3ccc(F)cc3C2=O)C(=O)N1 ; CC(C)(C)OC(=O)CCCN ; CC(C)(C)OC(=O)CCCNc1ccc2c(c1)C(=O)N(C1CCC(=O)NC1=O)C2=O ; O=C(O)CCCNc1ccc2c(c1)C(=O)N(C1CCC(=O)NC1=O)C2=O]

Handle 32 was synthesized following the same procedure as Handle 1 as described in Example 1 (0.52 g, yield: 25%). ¹H NMR (400 MHz, DMSO-d₆) δ 12.12 (s, 1H), 11.05 (s, 1H), 7.55 (d, J=8.4 Hz, 1H), 7.14 (t, J=4.8 Hz, 1H), 6.95 (d, J=2.0 Hz, 1H), 6.85 (dd, J=2.0, 8.4 Hz, 1H), 5.02 (dd, J=5.6, 12.8 Hz, 1H), 3.21-3.16 (m, 2H), 2.91-2.83 (m, 1H), 2.60-2.51 (m, 2H), 2.34 (t, J=7.2 Hz, 2H), 2.01-1.97 (m, 1H), 1.82-1.75 (m, 2H). MS (ESI) m/z=360.1 [M+H]⁺.

### Example 33: 5-((2-(2,6-Dioxopiperidin-3-yl)-1,3-dioxoisoindolin-5-yl)amino)pentanoic acid (Handle 33)

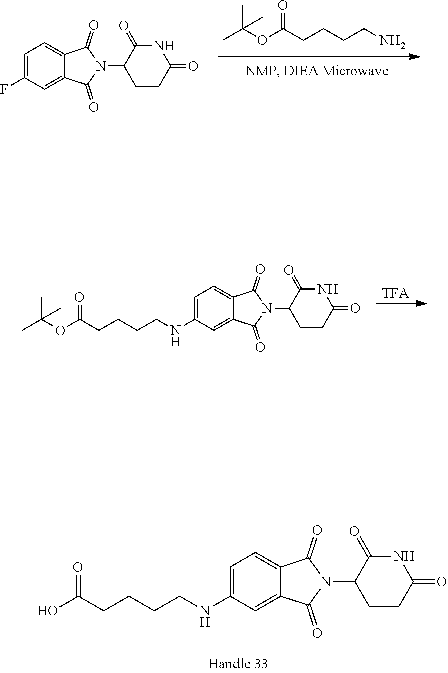 [SMILES: O=C1CCC(N2C(=O)c3ccc(F)cc3C2=O)C(=O)N1 ; CC(C)(C)OC(=O)CCCCN ; CC(C)(C)OC(=O)CCCCNc1ccc2c(c1)C(=O)N(C1CCC(=O)NC1=O)C2=O ; O=C(O)CCCCNc1ccc2c(c1)C(=O)N(C1CCC(=O)NC1=O)C2=O]

Handle 33 was synthesized following the same procedure as Handle 1 as described in Example 1 (0.66 g, yield: 51%). ¹H NMR (400 MHz, DMSO-d₆) δ 12.03 (brs, 1H), 11.05 (s, 1H), 7.55 (d, J=8.4 Hz, 1H), 7.10 (t, J=5.2 Hz, 1H), 6.94 (s, 1H), 6.83 (dd, J=1.6, 8.4 Hz, 1H), 5.02 (dd, J=5.6, 12.8 Hz, 1H), 3.17-3.16 (m, 2H), 2.92-2.83 (m, 1H), 2.60-2.53 (m, 2H), 2.26-2.25 (m, 2H), 2.01-1.98 (m, 1H), 1.60-1.59 (m, 4H). MS (ESI) m/z=374.1 [M+H]⁺.

### Example 34: 6-((2-(2,6-Dioxopiperidin-3-yl)-1,3-dioxoisoindolin-5-yl)amino)hexanoic acid Handle 34)

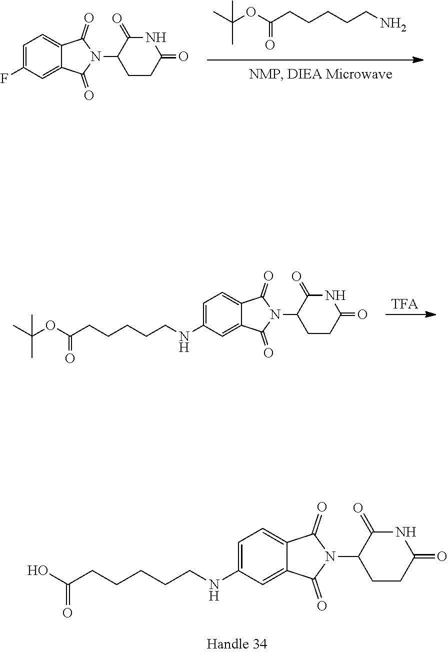 [SMILES: O=C1CCC(N2C(=O)c3ccc(F)cc3C2=O)C(=O)N1 ; CC(C)(C)OC(=O)CCCCCN ; CC(C)(C)OC(=O)CCCCCNc1ccc2c(c1)C(=O)N(C1CCC(=O)NC1=O)C2=O ; O=C(O)CCCCCNc1ccc2c(c1)C(=O)N(C1CCC(=O)NC1=O)C2=O]

Handle 34 was synthesized following the same procedure as Handle 1 as described in Example 1 (1.33 g, yield: 66%). ¹H NMR (400 MHz, DMSO-d₆) δ 11.98 (s, 1H), 11.05 (s, 1H), 7.55 (d, J=8.4 Hz, 1H), 7.08 (t, J=5.2 Hz, 1H), 6.95 (s, 1H), 6.83 (dd, J=1.2, 8.4 Hz, 1H), 5.03 (dd, J=5.2, 12.8 Hz, 1H), 3.17-3.12 (m, 2H), 2.92-2.83 (m, 1H), 2.60-2.53 (m, 2H), 2.22 (t, J=7.2 Hz, 2H), 2.01-1.98 (m, 1H), 1.61-1.51 (m, 4H), 1.41-1.33 (m, 2H). MS (ESI) m/z=388.1 [M+H]⁺.

### Example 35: 7-((2-(2,6-Dioxopiperidin-3-yl)-1,3-dioxoisoindolin-5-yl)amino)heptanoic acid (Handle 35)

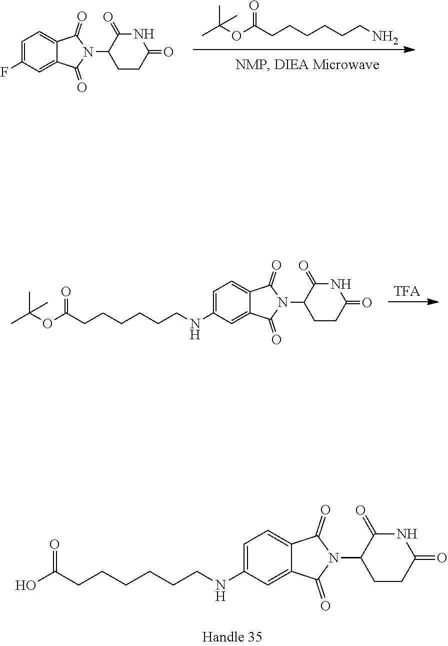 [SMILES: O=C1CCC(N2C(=O)c3ccc(F)cc3C2=O)C(=O)N1 ; CC(C)(C)OC(=O)CCCCCCN ; CC(C)(C)OC(=O)CCCCCCNc1ccc2c(c1)C(=O)N(C1CCC(=O)NC1=O)C2=O ; O=C(O)CCCCCCNc1ccc2c(c1)C(=O)N(C1CCC(=O)NC1=O)C2=O]

Handle 35 was synthesized following the same procedure as Handle 1 as described in Example 1 (1.06 g, yield: 39%). ¹H NMR (400 MHz, DMSO-d₆) δ 11.94 (s, 1H), 11.04 (s, 1H), 7.55 (d, J=8.4 Hz, 1H), 7.09 (t, J=5.6 Hz, 1H), 6.94 (d, J=2.0 Hz, 1H), 6.84 (dd, J=2.0, 8.4 Hz, 1H), 5.02 (dd, J=5.6, 13.2 Hz, 1H), 3.17-3.12 (m, 2H), 2.88-2.83 (m, 1H), 2.60-2.53 (m, 2H), 2.21 (t, J=7.2 Hz, 2H), 2.01-1.97 (m, 1H), 1.58-1.48 (m, 4H), 1.39-1.29 (m, 4H). MS (ESI) m/z=402.1 [M+H]⁺.

### Example 36: 8-((2-(2,6-Dioxopiperidin-3-yl)-1,3-dioxoisoindolin-5-yl)amino)octanoic acid (Handle 36)

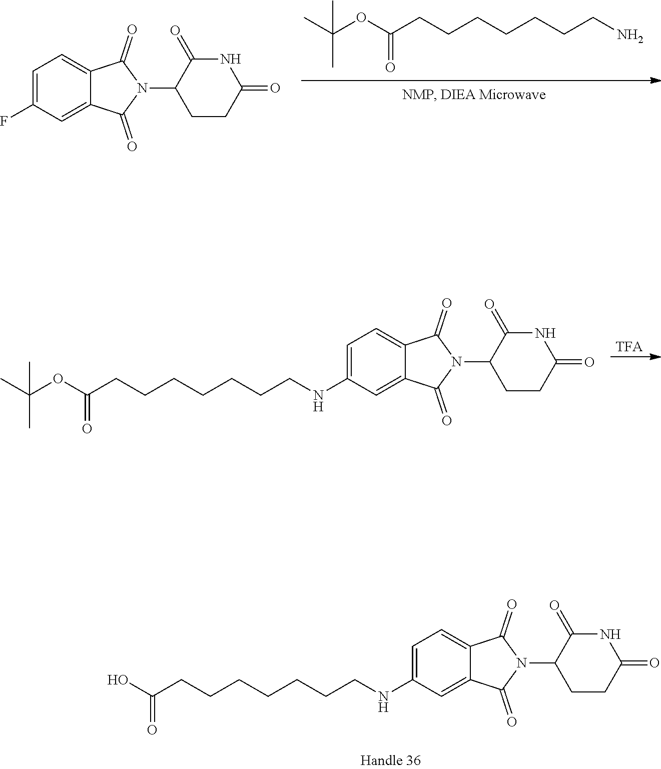 [SMILES: O=C1CCC(N2C(=O)c3ccc(F)cc3C2=O)C(=O)N1 ; CC(C)(C)OC(=O)CCCCCCCN ; CC(C)(C)OC(=O)CCCCCCCNc1ccc2c(c1)C(=O)N(C1CCC(=O)NC1=O)C2=O ; O=C(O)CCCCCCCNc1ccc2c(c1)C(=O)N(C1CCC(=O)NC1=O)C2=O]

Handle 36 was synthesized following the same procedure as Handle 1 as described in Example 1 (1.66 g, yield: 51%). ¹H NMR (400 MHz, DMSO-d₆) δ 11.95 (s, 1H), 11.05 (s, 1H), 7.55 (d, J=8.4 Hz, 1H), 7.09 (t, J=5.6 Hz, 1H), 6.94 (d, J=2.0 Hz, 1H), 6.84 (dd, J=2.0, 8.4 Hz, 1H), 5.02 (dd, J=5.6, 13.2 Hz, 1H), 3.17-3.12 (m, 2H), 2.88-2.83 (m, 1H), 2.60-2.53 (m, 2H), 2.19 (t, J=7.2 Hz, 2H), 2.02-1.98 (m, 1H), 1.58-1.47 (m, 4H), 1.36-1.29 (m, 6H). MS (ESI) m/z=416.1 [M+H]⁺.

### Example 37: 3-(2-((2-(2,6-Dioxopiperidin-3-yl)-1,3-dioxoisoindolin-5-yl)amino)ethoxy)propanoic acid (Handle 37)

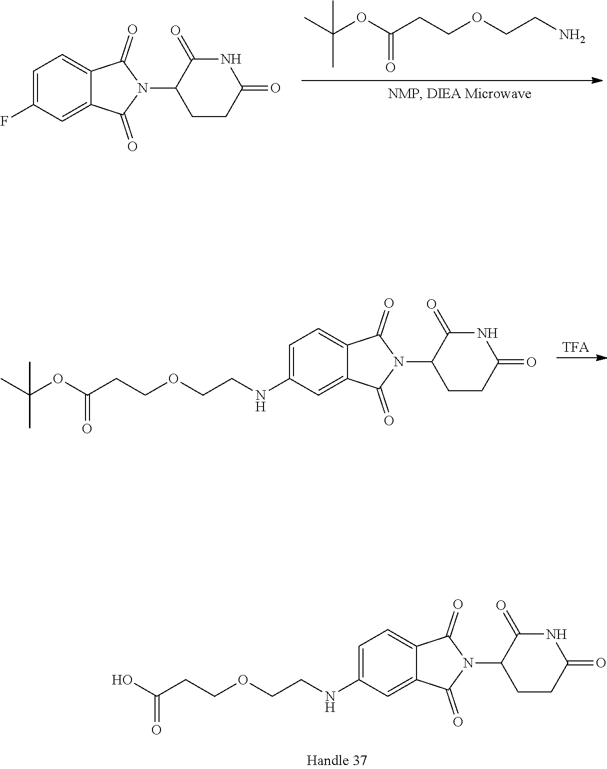 [SMILES: O=C1CCC(N2C(=O)c3ccc(F)cc3C2=O)C(=O)N1 ; CC(C)(C)OC(=O)CCOCCN ; CC(C)(C)OC(=O)CCOCCNc1ccc2c(c1)C(=O)N(C1CCC(=O)NC1=O)C2=O ; O=C(O)CCOCCNc1ccc2c(c1)C(=O)N(C1CCC(=O)NC1=O)C2=O]

Handle 37 was synthesized following the same procedure as Handle 1 as described in Example 1. (1.7 g, yield: 60%). ¹H NMR (400 MHz, DMSO-d₆) δ 12.19 (brs, 1H), 11.06 (s, 1H), 7.57 (d, J=8.4 Hz, 1H), 7.09 (brs, 1H), 7.01 (d, J=2.0 Hz, 1H), 6.90 (dd, J=2.0, 8.4 Hz, 1H), 5.04 (dd, J=5.6, 13.2 Hz, 1H), 3.66 (t, J=6.4 Hz, 2H), 3.59 (t, J=5.6 Hz, 2H), 3.35 (t, J=5.2 Hz, 2H), 2.93-2.84 (m, 1H), 2.62-2.56 (m, 2H), 2.52-2.47 (m, 2H), 2.03-1.99 (m, 1H). MS (ESI) m/z=390.1 [M+H]⁺.

### Example 38: 3-(2-(2-((2-(2,6-Dioxopiperidin-3-yl)-1,3-dioxoisoindolin-5-yl)amino)ethoxy)ethoxy)propanoic acid (Handle 38)

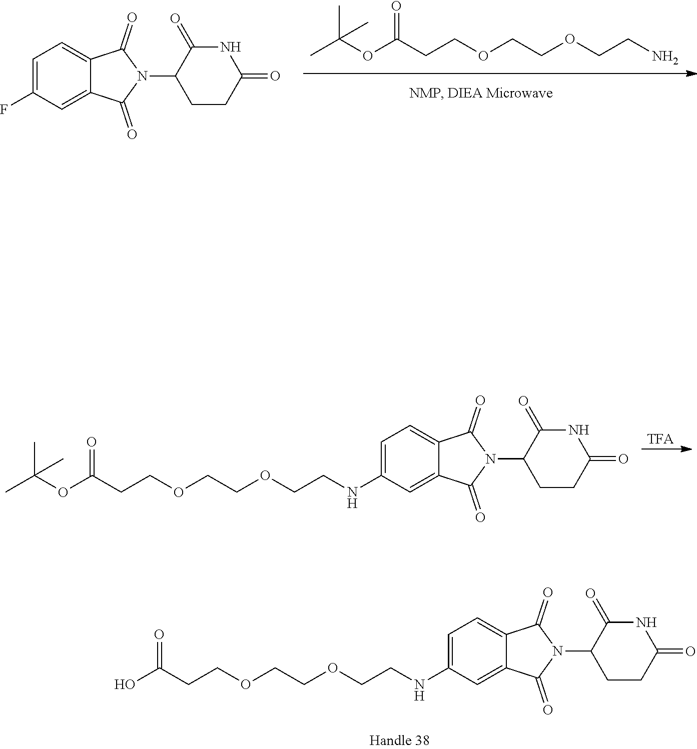

Handle 38 was synthesized following the same procedure as Handle 1 as described in Example 1 (2.3 g, yield: 78%). ¹H NMR (400 MHz, DMSO-d₆) δ 11.06 (s, 1H), 7.57 (d, J=8.4 Hz, 1H), 7.02 (d, J=2.0 Hz, 1H), 6.90 (dd, J=2.0, 8.4 Hz, 1H), 5.04 (dd, J=5.6, 13.2 Hz, 1H), 3.63-3.59 (m, 4H), 3.57-3.51 (m, 4H), 3.36 (t, J=5.6 Hz, 2H), 2.90-2.84 (m, 1H), 2.61-2.55 (m, 2H), 2.44 (t, J=6.4 Hz, 2H), 2.04-1.99 (m, 1H). MS (ESI) m/z=434.1 [M+H]⁺.

### Example 39: 3-(2-(2-(2-((2-(2,6-Dioxopiperidin-3-yl)-1,3-dioxoisoindolin-5-yl)amino)ethoxy)ethoxy)ethoxy)propanoic acid (Handle 39)

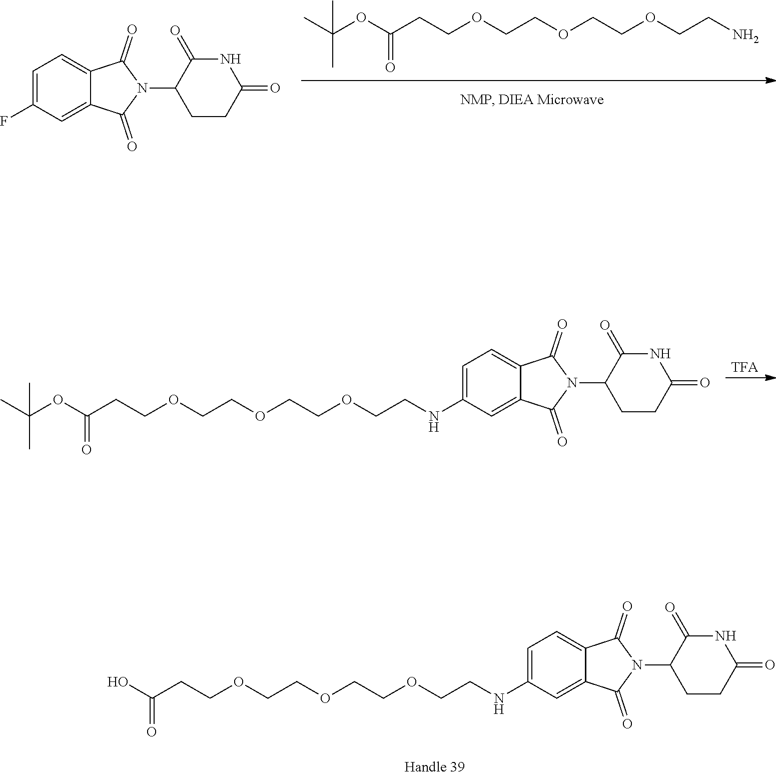 [SMILES: O=C1CCC(N2C(=O)c3ccc(F)cc3C2=O)C(=O)N1 ; CC(C)(C)OC(=O)CCOCCOCCOCCN ; CC(C)(C)OC(=O)CCOCCOCCOCCNc1ccc2c(c1)C(=O)N(C1CCC(=O)NC1=O)C2=O ; O=C(O)CCOCCOCCOCCNc1ccc2c(c1)C(=O)N(C1CCC(=O)NC1=O)C2=O]

Handle 39 was synthesized following the same procedure as Handle 1 as described in Example 1 (1.2 g, yield: 52%). ¹H NMR (400 MHz, DMSO-d₆) δ 7.59 (d, J=11.2 Hz, 1H), 7.23 (t, J=6.8 Hz, 1H), 7.04 (d, J=1.6 Hz, 1H), 7.04 (dd, J=2.4, 11.2 Hz, 1H), 5.06 (dd, J=7.2, 16.8 Hz, 1H), 3.64-3.57 (m, 8H), 3.54-3.48 (m, 4H), 3.40-3.38 (m, 2H), 2.92-2.89 (m, 1H), 2.64-2.54 (m, 2H), 2.42-2.38 (m, 2H), 2.05-2.01 (m, 1H). MS (ESI) m/z=478.1 [M+H]⁺.

### Example 40: 1-((2-(2,6-Dioxopiperidin-3-yl)-1,3-dioxoisoindolin-5-yl)amino)-3,6,9,12-tetraoxapentadecan-15-oic acid (Handle 40)

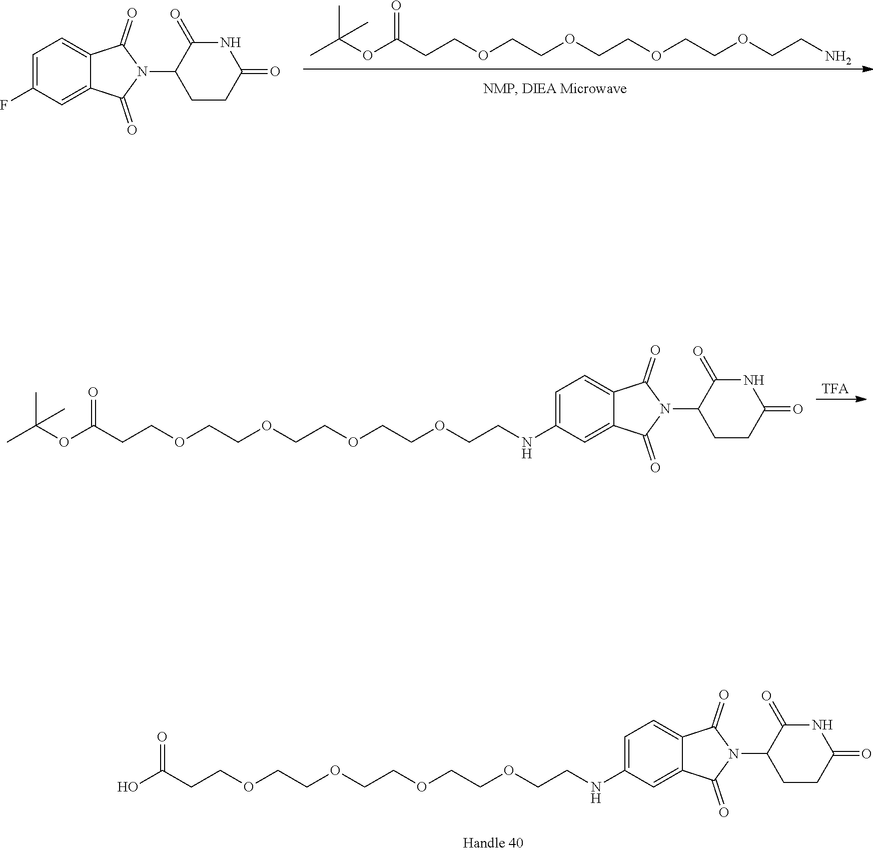

Handle 40 was synthesized following the same procedure as Handle 1 as described in Example 1 (1.3 g, yield: 55%). ¹H NMR (400 MHz, DMSO-d₆) δ 12.17 (brs, 1H), 11.07 (s, 1H), 7.56 (d, J=8.4 Hz, 1H), 7.17 (t, J=5.6 Hz, 1H), 7.01 (d, J=1.2 Hz, 1H), 6.90 (dd, J=1.6, 8.4 Hz, 1H), 5.03 (dd, J=5.6, 12.8 Hz, 1H), 3.61-3.48 (m, 18H), 2.92-2.83 (m, 1H), 2.60-2.54 (m, 2H), 2.43 (t, J=6.4 Hz, 2H), 2.03-1.98 (m, 1H). MS (ESI) m/z=522.1 [M+H]⁺.

### Example 41: 1-((2-(2,6-Dioxopiperidin-3-yl)-1,3-dioxoisoindolin-5-yl)amino)-3,6,9,12,15-pentaoxaoctadecan-18-oic acid (Handle 41)

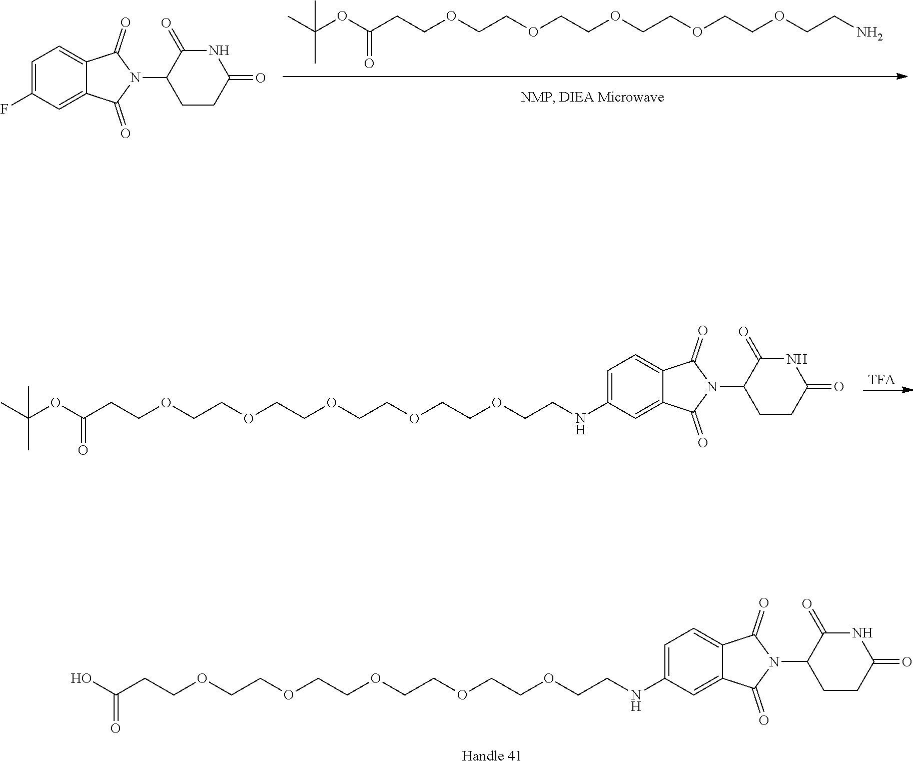 [SMILES: O=C1CCC(N2C(=O)c3ccc(F)cc3C2=O)C(=O)N1 ; CC(C)(C)OC(=O)CCOCCOCCOCCOCCOCCN ; CC(C)(C)OC(=O)CCOCCOCCOCCOCCOCCNc1ccc2c(c1)C(=O)N(C1CCC(=O)NC1=O)C2=O ; O=C(O)CCOCCOCCOCCOCCOCCNc1ccc2c(c1)C(=O)N(C1CCC(=O)NC1=O)C2=O]

Handle 41 was synthesized following the same procedure as Handle 1 as described in Example 1 (1.0 g, yield: 50%). ¹H NMR (400 MHz, DMSO-d₆) δ 12.17 (brs, 1H), 11.07 (s, 1H), 7.56 (d, J=8.0 Hz, 1H), 7.17 (t, J=5.6 Hz, 1H), 7.01 (s, 1H), 6.90 (dd, J=1.6, 8.4 Hz, 1H), 5.03 (dd, J=5.6, 13.2 Hz, 1H), 3.60-3.48 (m, 22H), 2.89-2.83 (m, 1H), 2.60-2.54 (m, 2H), 2.43 (t, J=6.4 Hz, 2H), 2.01-1.98 (m, 1H). MS (ESI) m/z=566.1 [M+H]⁺.

### Procedures for the Synthesis of TYK2 Binding Moiety

### Example 42: 4-((3-(1-(2-Aminoethyl)-1H-1,2,4-triazol-3-yl)-2-methoxyphenyl)amino)-6-(cyclopropanecarboxamido)-N-methylpyridazine-3-carboxamide

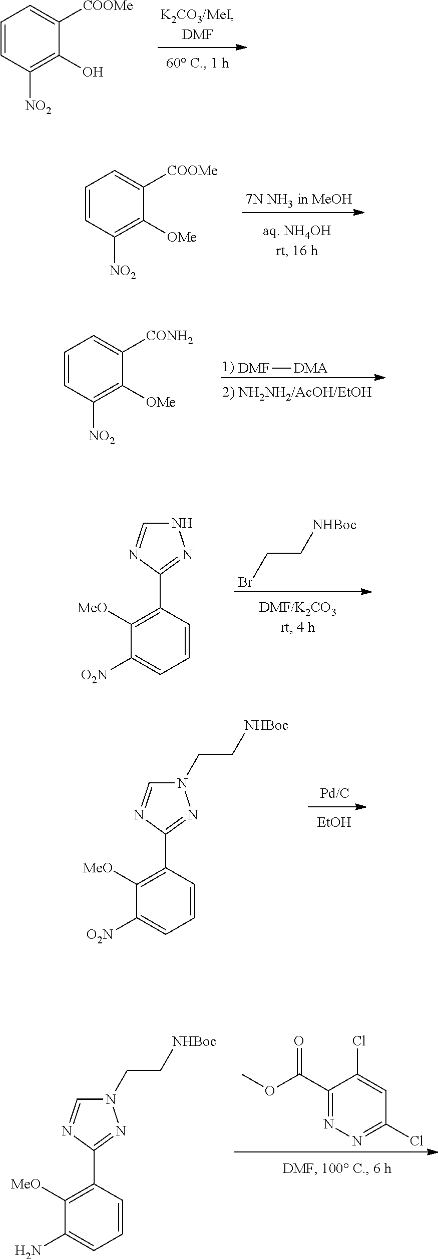

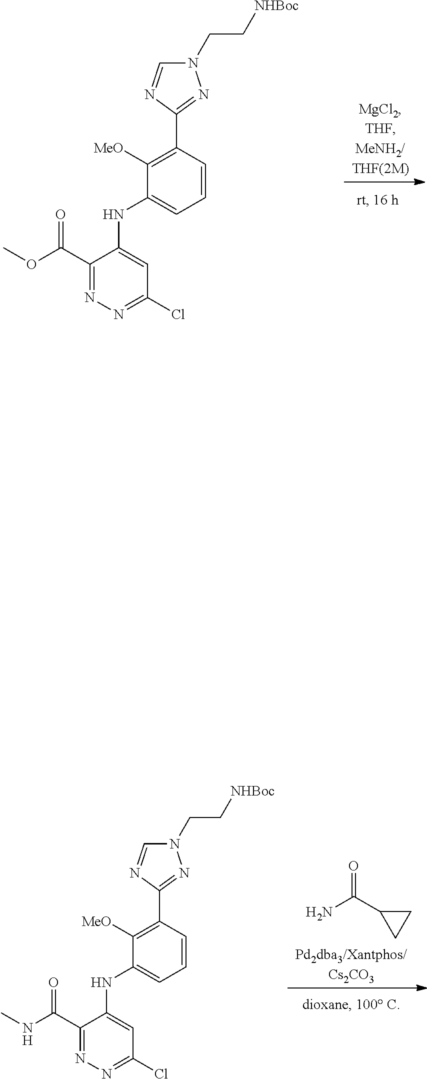 [SMILES: COC(=O)c1nnc(Cl)cc1Nc1cccc(-c2ncn(CCNC(=O)OC(C)(C)C)n2)c1OC ; CNC(=O)c1nnc(Cl)cc1Nc1cccc(-c2ncn(CCNC(=O)OC(C)(C)C)n2)c1OC ; NC(=O)C1CC1 ; "dioxane, 100° C."]

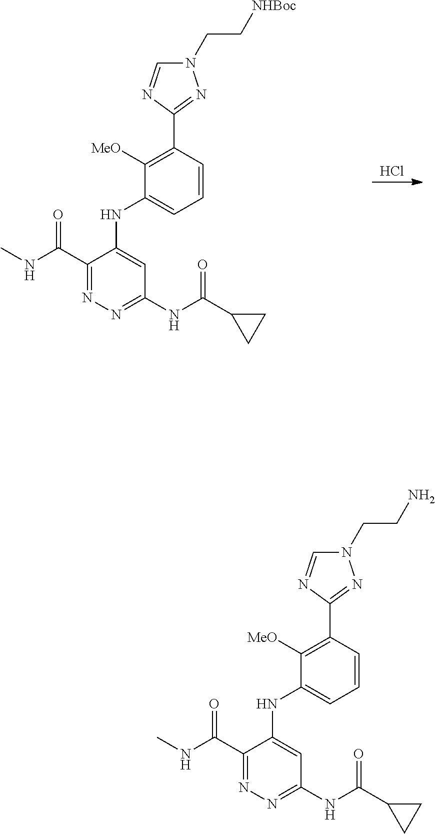 [SMILES: CNC(=O)c1nnc(NC(=O)C2CC2)cc1Nc1cccc(-c2ncn(CCNC(=O)OC(C)(C)C)n2)c1OC ; Cl ; CNC(=O)c1nnc(NC(=O)C2CC2)cc1Nc1cccc(-c2ncn(CCN)n2)c1OC]

### Step 1. Synthesis of methyl 2-methoxy-3-nitrobenzoate

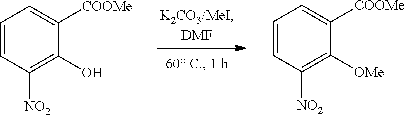 [SMILES: COC(=O)c1cccc([N+](=O)[O-])c1O ; COC(=O)c1cccc([N+](=O)[O-])c1OC]

To a solution of methyl 2-hydroxy-3-nitrobenzoate (160 g, 812.2 mmol) in DMF (2.5 L) were added K₂CO₃ (224.2 g, 1624.4 mmol) and CH₃I (230.6 g, 1624.4 mmol) at room temperature. The mixture was stirred at 60° C. for 1 h. After cooling down to rt, the mixture was diluted with water (3.0 L) and extracted with EtOAc (0.6 L×5). The combined organic layers were washed with brine (1.0 L) and dried over Na₂SO₄, filtered, and concentrated to give the title compound (170 g, 99.4% yield) as a yellow solid which was used in the next step without further purification. ¹H NMR (400 MHz, DMSO-d₆) δ 8.13 (dd, J=1.6, 8.0 Hz, 1H), 8.04 (dd, J=2.0, 8.0 Hz, 1H), 7.45 (t, J=7.8 Hz, 1H), 3.90 (s, 3H), 3.88 (s, 3H).

### Step 2. Synthesis of 2-methoxy-3-nitrobenzamide

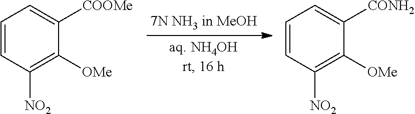 [SMILES: COC(=O)c1cccc([N+](=O)[O-])c1OC ; COc1c(C(N)=O)cccc1[N+](=O)[O-]]

Methyl 2-methoxy-3-nitrobenzoate (170 g, 805.7 mmol) was dissolved in a cold solution of ammonia in methanol (7 N, 3.0 L) and concentrated ammonium hydroxide (0.6 L). The mixture was stirred at room temperature for 16 h. The mixture was concentrated, and the residue was diluted with water (0.8 L). The mixture was sonicated and filtered. The filter cake was washed with ice cold water (1.0 L) to give the title compound (150 g, 94.9% yield) as an orange solid. ¹H NMR (400 MHz, DMSO-d₆) δ 7.99-7.94 (m, 2H), 7.76 (dd, J=7.6, 1.6 Hz, 2H), 7.37 (t, J=8.0 Hz, 1H), 3.88 (s, 3H).

### Step 3. Synthesis of 3-(2-methoxy-3-nitrophenyl)-1H-1,2,4-triazole

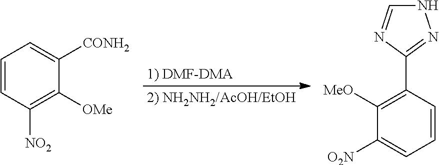 [SMILES: COc1c(C(N)=O)cccc1[N+](=O)[O-] ; COc1c(-c2nc[nH]n2)cccc1[N+](=O)[O-]]

A solution of 2-methoxy-3-nitrobenzamide (150 g, 765.3 mmol) in DMF-DMA (1.1 L) was stirred at 95° C. for 30 min. TLC (petroleum ether/EtOAc=1/1) showed the reaction was completed. The mixture was concentrated and azeotroped with 1,2-dichloroethane (0.5 L) to ensure complete removal of any residue DMF-DMA. The crude oil was dissolved in EtOH (0.5 L) and added to a mixture of hydrazine hydrate (0.4 L) in EtOH (3.0 L) and CH₃COOH (0.8 L) at 0° C. After the addition was completed, the mixture was warmed to rt and stirred for 4h. The mixture was concentrated, and the residue was sonicated with water (1.0 L) and filtered. The filter cake was washed with ice water (1.0 L) to give the title compound (155 g, 91.9% yield) as a pale-yellow solid which was used in the next step without further purification. ¹H NMR (400 MHz, DMSO-d₆) δ 14.33 (brs, 1H), 8.71 (brs, 1H), 8.22 (d, J=7.2 Hz, 1H), 7.98 (brs, 1H), 7.46 (t, J=8.0 Hz, 1H), 3.80 (s, 3H).

### Step 4. Synthesis of tert-butyl (2-(3-(2-methoxy-3-nitrophenyl)-1H-1,2,4-triazol-1-yl)ethyl)carbamate

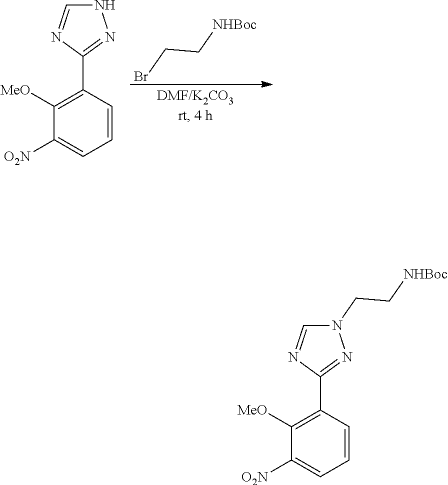

To a solution of 3-(2-methoxy-3-nitrophenyl)-1H-1,2,4-triazole (13 g, 59.0 mmol) in DMF (150 mL) were added K₂CO₃ (24.4 g, 177.0 mmol) and a solution of tert-butyl (2-bromoethyl)carbamate (19.7 g, 88.5 mmol) in DMF (30 mL) dropwise at 0° C. After the mixture was stirred at rt for 16 h, it was diluted with water (200 mL) and extracted with EtOAc (150 mL×3). The combined organic layers were washed with brine (200 mL), dried over Na₂SO₄, filtered, and concentrated under reduced pressure. The residue was purified by silica gel column chromatography (petroleum ether/EtOAc=10:1 to 1:3) to give the title compound (20 g, 93.2% yield) as a yellow solid. MS (ESI) m/z=364.1 [M+H]⁺.

### Step 5. Synthesis of tert-butyl (2-(3-(3-amino-2-methoxyphenyl)-1H-1,2,4-triazol-1-yl)ethyl)carbamate

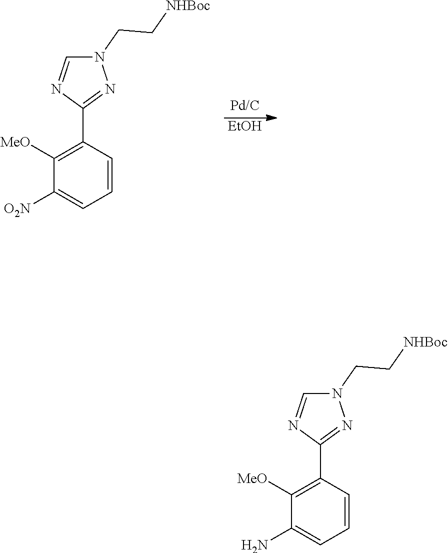 [SMILES: "EtOH" ; COc1c(-c2ncn(CCNC(=O)OC(C)(C)C)n2)cccc1[N+](=O)[O-] ; COc1c(N)cccc1-c1ncn(CCNC(=O)OC(C)(C)C)n1]

A mixture of tert-butyl (2-(3-(2-methoxy-3-nitrophenyl)-1H-1,2,4-triazol-1-yl)ethyl)carbamate (20 g, 55.1 mmol) and Pd/C (5 g) in EtOH (200 mL) was stirred at rt for 5 h under H₂. The mixture was filtered through Celite. The filtrate was concentrated under reduced pressure to give the title compound (17 g, 92.9% yield) as colorless oil which was used in the next step without further purification. MS (ESI) m/z=334.2 [M+H]⁺.

### Step 6. Synthesis of methyl 4-((3-(1-(2-((tert-butoxycarbonyl)amino)ethyl)-1H-1,2,4-triazol-3-yl)-2-methoxyphenyl)amino)-6-chloropyridazine-3-carboxylate

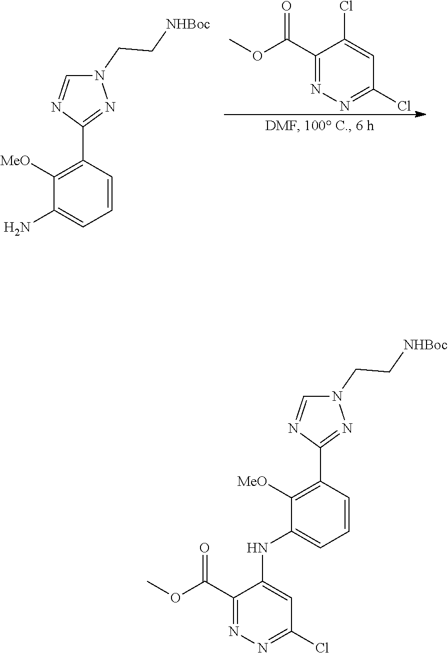 [SMILES: COc1c(N)cccc1-c1ncn(CCNC(=O)OC(C)(C)C)n1 ; COC(=O)c1nnc(Cl)cc1Cl ; COC(=O)c1nnc(Cl)cc1Nc1cccc(-c2ncn(CCNC(=O)OC(C)(C)C)n2)c1OC]

A mixture of tert-butyl (2-(3-(3-amino-2-methoxyphenyl)-1H-1,2,4-triazol-1-yl)ethyl)carbamate (5.8 g, 17.4 mmol) and methyl 4,6-dichloropyridazine-3-carboxylate (4.30 g, 20.88 mmol) in DMF (5 mL) was stirred at 100° C. for 6 h. After cooling down to rt, the mixture was diluted with water (80 mL) and extracted with EtOAc (50 mL×3). The combined organic layers were washed with brine (80 mL), dried over Na₂SO₄, filtered, and concentrated under reduced pressure. The residue was purified by silica gel column chromatography (DCM/MeOH=200:1 to 60:1) to give the title compound (4.2 g, 48.0% yield) as a yellow solid. MS (ESI) m/z=504.1 [M+H]⁺.

### Step 7. Synthesis of tert-butyl (2-(3-(3-((6-chloro-3-(methylcarbamoyl)pyridazin-4-yl)amino)-2-methoxyphenyl)-1H-1,2,4-triazol-1-yl)ethyl)carbamate

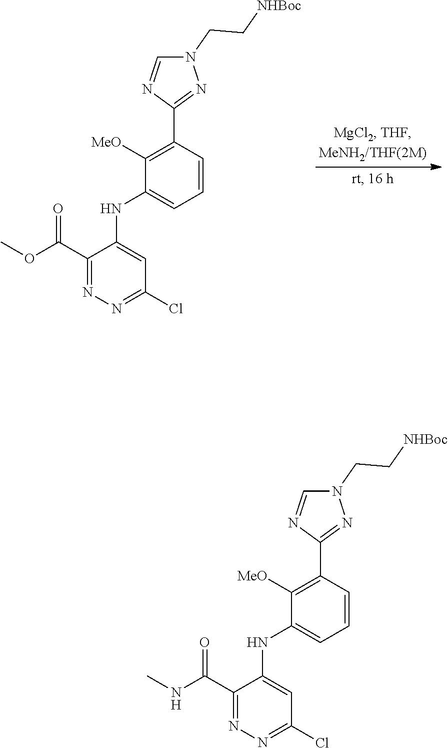 [SMILES: COC(=O)c1nnc(Cl)cc1Nc1cccc(-c2ncn(CCNC(=O)OC(C)(C)C)n2)c1OC ; CNC(=O)c1nnc(Cl)cc1Nc1cccc(-c2ncn(CCNC(=O)OC(C)(C)C)n2)c1OC]

To a solution of methyl 4-((3-(1-(2-((tert-butoxycarbonyl)amino)ethyl)-1H-1,2,4-triazol-3-yl)-2-methoxyphenyl)amino)-6-chloropyridazine-3-carboxylate (4.2 g, 8.35 mmol) in THF (50 mL) was added magnesium chloride (398.0 mg, 4.18 mmol). After the mixture was stirred at rt for 5 min, methylamine (2M in THF, 10 mL) solution was added. The mixture was stirred at rt for 16 h. The mixture was diluted with water (30 mL) and extracted with EtOAC (20 mL×3). The combined organic layers were washed with brine (30 mL), dried over Na₂SO₄, filtered, and concentrated under reduced pressure. The residue was purified by silica gel column chromatography (DCM/MeOH=200:1 to 60:1) to give the title compound (3.8 g, 90.6% yield) as a yellow solid. ¹H NMR (400 MHz, DMSO-d₆) δ 11.13 (s, 1H), 9.40 (d, J=4.8 Hz, 1H), 8.54 (s, 1H), 7.74 (d, J=6.8 Hz, 1H), 7.61 (dd, J=8.0, 1.2 Hz, 1H), 7.29 (t, J=8.0 Hz, 1H), 7.20 (s, 1H), 7.05-6.98 (m, 1H), 4.28 (t, J=6.0 Hz, 2H), 3.71 (s, 3H), 3.42-3.37 (m, 2H), 2.87 (d, J=4.8 Hz, 3H), 1.35 (s, 9H).

### Step 8. Synthesis of tert-butyl (2-(3-(3-((6-(cyclopropanecarboxamido)-3-(methylcarbamoyl)pyridazin-4-yl)amino)-2-methoxyphenyl)-1H-1,2,4-triazol-1-yl)ethyl)carbamate

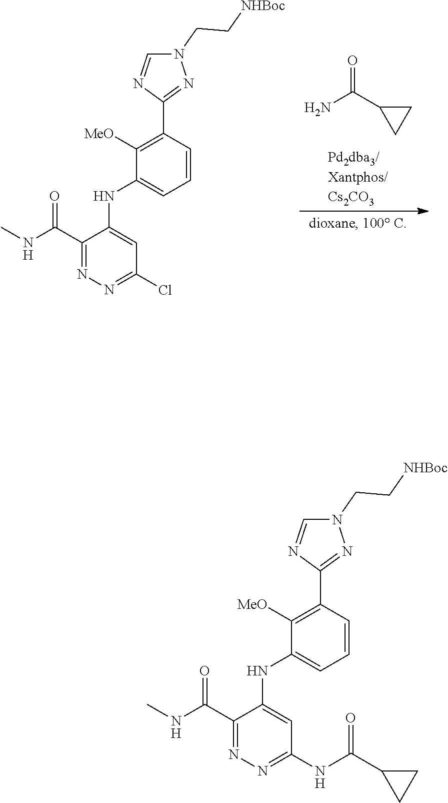 [SMILES: CNC(=O)c1nnc(Cl)cc1Nc1cccc(-c2ncn(CCNC(=O)OC(C)(C)C)n2)c1OC ; NC(=O)C1CC1 ; CNC(=O)c1nnc(NC(=O)C2CC2)cc1Nc1cccc(-c2ncn(CCNC(=O)OC(C)(C)C)n2)c1OC]

A mixture of tert-butyl (2-(3-(3-((6-chloro-3-(methylcarbamoyl)pyridazin-4-yl)amino)-2-methoxyphenyl)-1H-1,2,4-triazol-1-yl)ethyl)carbamate (3.8 g, 7.57 mmol), cyclopropanecarboxamide (1.29 g, 15.14 mmol), Pd₂(dba)₃ (351.5 mg, 0.38 mmol), XantPhos (440.8 mg, 0.76 mmol) and Cs₂CO₃ (4.92 g, 15.14 mmol) in dioxane (40 mL) was stirred at 100° C. for 72 h under N₂. LCMS showed about 50% conversion. After cooling down to rt, the mixture was concentrated under reduced pressure. The residue was purified by silica gel column chromatography (DCM/MeOH=200:1 to 40:1) to give the title compound (1.25 g, 29.9% Yield) as a green solid. MS (ESI) m₇z=552.7 [M+H]⁺.

### Step 9. Synthesis of 4-((3-(1-(2-aminoethyl)-1H-1,2,4-triazol-3-yl)-2-methoxyphenyl)amino)-6-(cyclopropanecarboxamido)-N-methylpyridazine-3-carboxamide

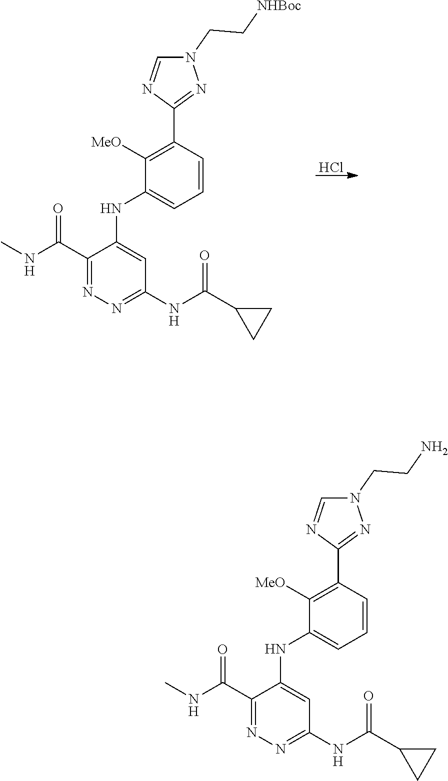 [SMILES: CNC(=O)c1nnc(NC(=O)C2CC2)cc1Nc1cccc(-c2ncn(CCNC(=O)OC(C)(C)C)n2)c1OC ; Cl ; CNC(=O)c1nnc(NC(=O)C2CC2)cc1Nc1cccc(-c2ncn(CCN)n2)c1OC]

A solution of tert-butyl (2-(3-(3-((6-(cyclopropanecarboxamido)-3-(methylcarbamoyl)pyridazin-4-yl)amino)-2-methoxyphenyl)-1H-1,2,4-triazol-1-yl)ethyl)carbamate (1.25 g, 2.26 mmol) in HCl/EtOAc (10 mL, 3M) was stirred at rt for 2 h. The mixture was concentrated and diluted with saturated sodium bicarbonate aqueous solution (100 mL). After stirring at rt for 3 h, the suspension was filtered. The filter cake was washed with water, and dried to give the title compound (860 mg, 84.5% yield) as a pale yellow solid. ¹H NMR (400 MHz, DMSO-d₆) δ 11.33 (brs, 1H), 10.98 (s, 1H), 9.17 (d, J=4.8 Hz, TH), 8.58 (s, 1H), 8.16 (s, 1H), 7.68 (d, J=6.4 Hz, 1H), 7.51 (d, J=6.8 Hz, 1H), 7.27 (t, J=8.0 Hz, 1H), 4.20 (t, J=6.0 Hz, 2H), 3.72 (s, 3H), 2.98 (t, J=6.0 Hz, 2H), 2.86 (d, J=4.8 Hz, 3H), 2.10-2.07 (m, 1H), 1.58 (s, 1H), 0.82 (d, J=5.2 Hz, 4H). MS (ESI) m/z=452.2 [M+H]⁺.

### Example 43: 6-(2-Aminoacetamido)-4-((2-methoxy-3-(1-methyl-1H-1,2,4-triazol-3-yl)phenyl)amino)-N-methylpyridazine-3-carboxamide

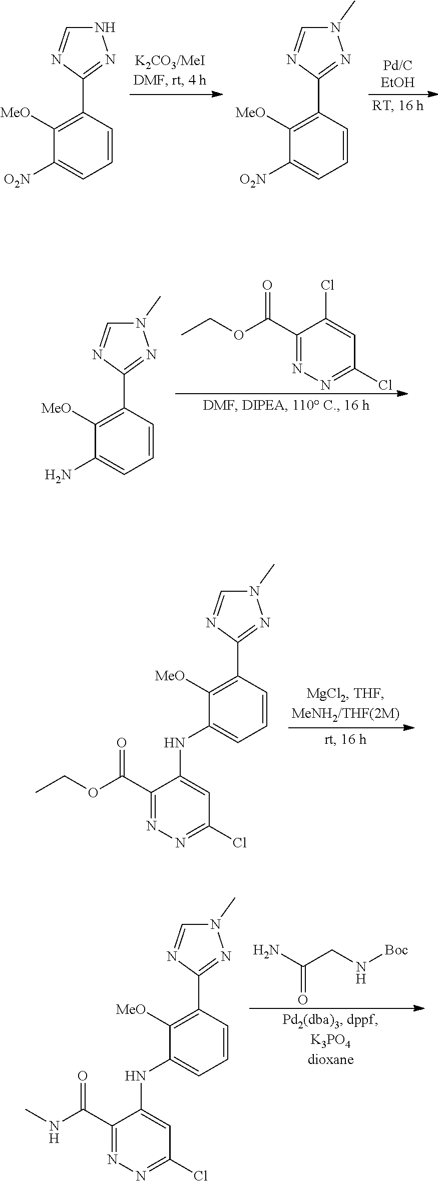

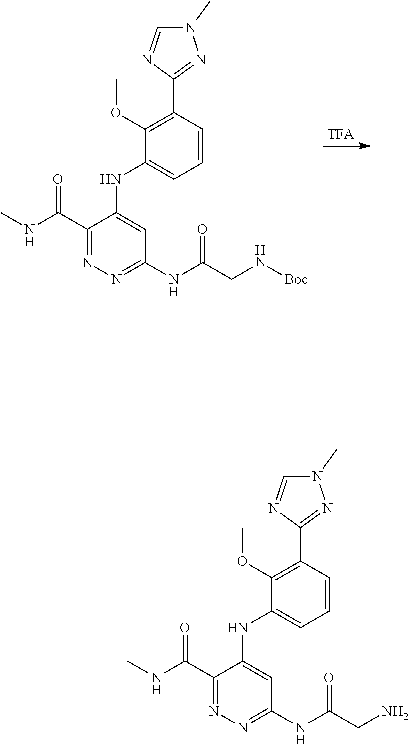 [SMILES: CNC(=O)c1nnc(NC(=O)CNC(=O)OC(C)(C)C)cc1Nc1cccc(-c2ncn(C)n2)c1OC ; CNC(=O)c1nnc(NC(=O)CN)cc1Nc1cccc(-c2ncn(C)n2)c1OC]

### Step 1. Synthesis of 3-(2-methoxy-3-nitrophenyl)-1-methyl-1H-1,2,4-triazole

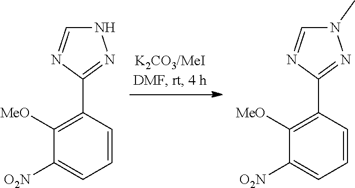 [SMILES: COc1c(-c2nc[nH]n2)cccc1[N+](=O)[O-] ; COc1c(-c2ncn(C)n2)cccc1[N+](=O)[O-]]

To a solution of 3-(2-methoxy-3-nitrophenyl)-1H-1,2,4-triazole (50 g, 227.3 mmol) in DMF (600 mL) were added K₂CO₃ (94.1 g, 681.9 mmol) and a solution of CH₃I (48.4 g, 341.0 mmol) in DMF (50 mL) dropwise at 0° C. After the mixture was stirred at rt for 4 h, it was diluted with water (800 mL) and extracted with EtOAc (300 mL×3). The combined organic layers were washed with brine (500×2 mL), dried over Na₂SO₄, filtered, and concentrated under reduced pressure. The residue was purified by silica gel column chromatography (petroleum ether/EtOAc=10:1 to 1:2) to give the title compound (29.4 g, 55.4% yield) as an off-white solid. ¹H NMR (400 MHz, DMSO-d₆) δ 8.64 (s, 1H), 8.17 (dd, J=8.0, 1.6 Hz, 1H), 7.94 (dd, J=8.0, 1.6 Hz, 1H), 7.43 (t, J=8.0 Hz, 1H), 3.97 (s, 3H), 3.83 (s, 3H).

### Step 2. Synthesis of 2-methoxy-3-(1-methyl-1H-1,2,4-triazol-3-yl)aniline

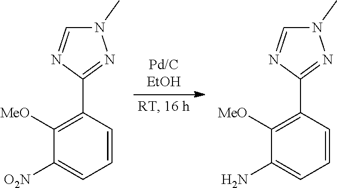 [SMILES: COc1c(-c2ncn(C)n2)cccc1[N+](=O)[O-] ; COc1c(N)cccc1-c1ncn(C)n1]

A solution of 3-(2-methoxy-3-nitrophenyl)-1-methyl-1H-1,2,4-triazole (20 g, 85.5 mmol) and Pd/C (5.0 g) in EtOH (200 mL) was stirred at rt for 5 h under H₂. LCMS showed the reaction was completed. The mixture was filtered through Celite. The filtrate was concentrated to give the title compound (17.2 g, 98.8% yield) as a white solid which was used in the next step without further purification. ¹H NMR (400 MHz, DMSO-d₆) δ 8.47 (s, 1H), 6.95 (dd, J=7.6, 1.6 Hz, 1H), 6.86 (t, J=7.8 Hz, 1H), 6.74 (dd, J=7.6, 1.6 Hz, 1H), 4.98 (s, 2H), 3.91 (s, 3H), 3.66 (s, 3H).

### Step 3. Synthesis of ethyl 6-chloro-4-((2-methoxy-3-(1-methyl-1H-1,2,4-triazol-3-yl)phenyl)amino)pyridazine-3-carboxylate

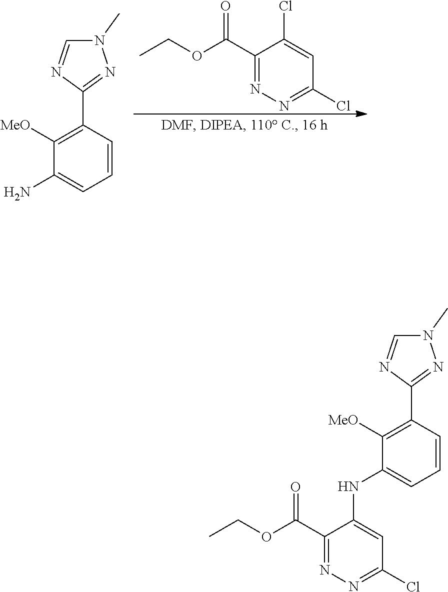

A mixture of 2-methoxy-3-(1-methyl-1H-1,2,4-triazol-3-yl)aniline (17.2 g, 84.3 mmol), ethyl 4,6-dichloropyridazine-3-carboxylate (18.5 g, 84.3 mmol) and DIPEA (21.7 g, 168.6 mmol) in DMF (300 mL) was stirred at 110° C. for 16 h. After cooling down to rt, the mixture was diluted with water (500 mL) and extracted with EtOAc (300 mL×5). The combined organic layers were washed with brine (500 mL×2), dried over Na₂SO₄, filtered, and concentrated under reduced pressure. The residue was purified by silica gel column chromatography (DCM/MeOH=100:1 to 10:1) to give the title compound (16.8 g, 51.4% yield) as a yellow solid.

### Step 4. Synthesis of 6-chloro-4-((2-methoxy-3-(1-methyl-1H-1,2,4-triazol-3-yl)phenyl)amino)-N-methylpyridazine-3-carboxamide

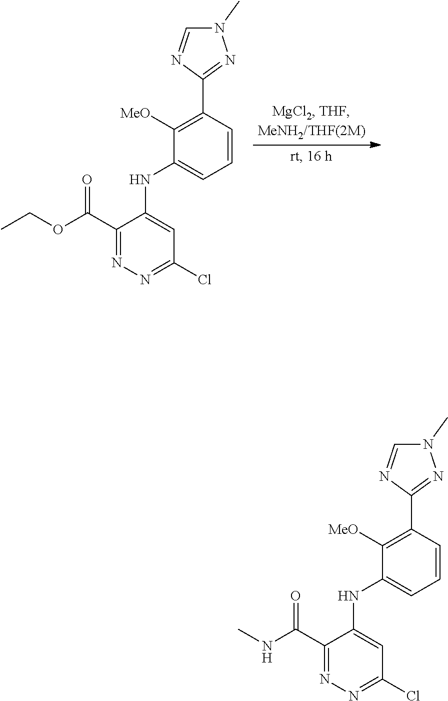

To a solution of ethyl 6-chloro-4-((2-methoxy-3-(1-methyl-1H-1,2,4-triazol-3-yl)phenyl)amino)pyridazine-3-carboxylate (15.5 g, 40.1 mmol) in THF (600 mL) was added magnesium chloride (1.9 g, 20.1 mmol). After stirring at rt for 5 min, methylamine/THF solution (50 mL, 2 M) was added. The mixture was stirred at rt for 16 h. LCMS showed the reaction was completed. The mixture was quenched with 1 M HCl (30 mL), diluted with water (300 mL) and extracted with EtOAc (150 mL×5). The combined organic layers were washed with brine (300 mL), dried over Na₂SO₄, filtered, and concentrated under reduced pressure. The residue was purified by silica gel column chromatography (DCM/MeOH=100:1 to 10:1) to give the title compound (13.7 g, 91.9% yield) as a grey white solid. ¹H NMR (400 MHz, DMSO-d₆) δ 11.11 (s, 1H), 9.39 (d, J=5.2 Hz, 1H), 8.57 (s, 1H), 7.72 (dd, J=7.6, 1.6 Hz, 1H), 7.60 (dd, J=8.0, 1.6 Hz, 1H), 7.29 (t, J=8.0 Hz, 1H), 7.20 (s, 1H), 3.95 (s, 3H), 3.72 (s, 3H), 2.87 (d, J=4.8 Hz, 3H). MS (ESI) m/z=374.1 [M+H]⁺.

### Step 5. Synthesis of tert-butyl (2-((5-((2-methoxy-3-(1-methyl-1H-1,2,4-triazol-3-yl)phenyl)amino)-6-(methylcarbamoyl)pyridazin-3-yl)amino)-2-oxoethyl)carbamate

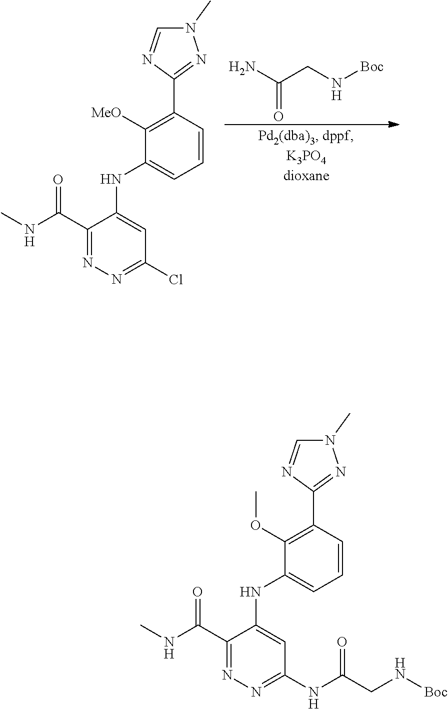

A mixture of 6-chloro-4-((2-methoxy-3-(1-methyl-1H-1,2,4-triazol-3-yl)phenyl)amino)-N-methylpyridazine-3-carboxamide (1.5 g, 4.02 mmol) and K₃PO₄ (2.56 g, 12.06 mmol) in dioxane (10 mL) were added tert-butyl (2-amino-2-oxoethyl)carbamate (1.05 g, 6.03 mmol), Pd₂(dba)₃ (37 mg, 0.04 mmol) and dppf (44 mg, 0.08 mmol). The resulting mixture was degassed with N₂ and stirred at 100° C. for 16 h. After cooling down to rt, the mixture was diluted with water and extracted with EtOAc (40 mL×3). The combined organic layers were washed with brine (40 mL×2), dried over Na₂SO₄, filtered and concentrated under reduced pressure. The resulting residue was purified by reverse-phase chromatography to give the title compound (1.05 g, yield: 51.2%) as an off-white solid. MS (ESI) m/z=512.6 [M+H]⁺.

### Step 6. Synthesis of 6-(2-aminoacetamido)-4-((2-methoxy-3-(1-methyl-1H-1,2,4-triazol-3-yl)phenyl)amino)-N-methylpyridazine-3-carboxamide

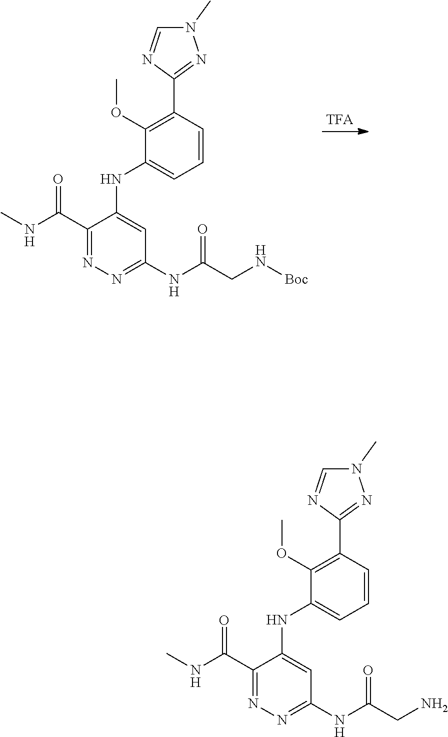 [SMILES: CNC(=O)c1nnc(NC(=O)CNC(=O)OC(C)(C)C)cc1Nc1cccc(-c2ncn(C)n2)c1OC ; CNC(=O)c1nnc(NC(=O)CN)cc1Nc1cccc(-c2ncn(C)n2)c1OC]

To a solution of tert-butyl (2-((5-((2-methoxy-3-(1-methyl-1H-1,2,4-triazol-3-yl)phenyl)amino)-6-(methylcarbamoyl)pyridazin-3-yl)amino)-2-oxoethyl)carbamate (1.05 g, 2.05 mmol) in DCM (15 mL) was added TFA (15 mL). The resulting mixture was stirred at 25° C. for 16 h. The solvents were removed under reduced pressure. The resulting residue was purified by reverse-phase chromatography to give the title compound (822 mg, yield: 97.4%) as an off-white solid. MS (ESI) m/z=412.6 [M+H]⁺.

### Procedures for the Synthesis of Heterobifunctional Compounds

### Example 44: 6-(Cyclopropanecarboxamido)-4-((3-(1-(2-(2-((2-(2,6-dioxopiperidin-3-yl)-1,3-dioxoisoindolin-4-yl)amino)acetamido)ethyl)-1H-1,2,4-triazol-3-yl)-2-methoxyphenyl)amino)-N-methylpyridazine-3-carboxamide (CPD-001)

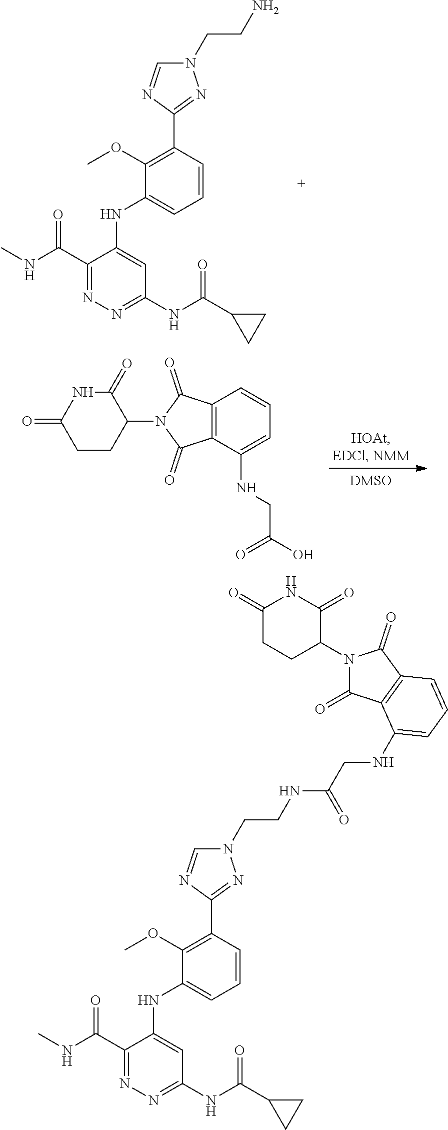

CPD-001 was synthesized following the same procedure for preparing CPD-042 (2.8 mg, yield: 33%). MS (ESI) m/z=765.6 [M+H]⁺.

### Example 45: 6-(Cyclopropanecarboxamido)-4-((3-(1-(2-(3-((2-(2,6-dioxopiperidin-3-yl)-1,3-dioxoisoindolin-4-yl)amino)propanamido)ethyl)-1H-1,2,4-triazol-3-yl)-2-methoxyphenyl)amino)-N-methylpyridazine-3-carboxamide (CPD-002)

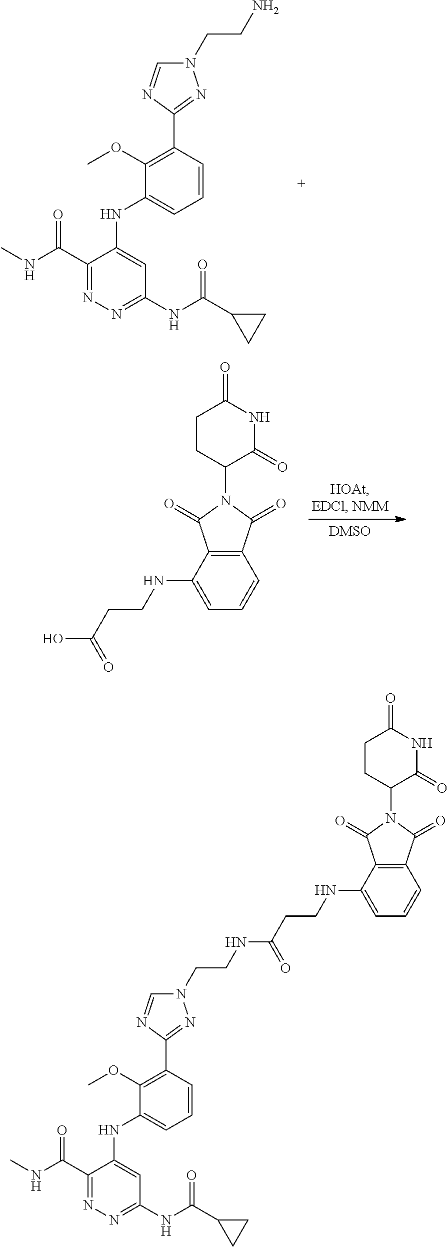

CPD-002 was synthesized following the same procedure for preparing CPD-042 (2.33 mg, yield: 27%). MS (ESI) m/z=779.6 [M+H]⁺.

### Example 46: 6-(Cyclopropanecarboxamido)-4-((3-(1-(2-(4-((2-(2,6-dioxopiperidin-3-yl)-1,3-dioxoisoindolin-4-yl)amino)butanamido)ethyl)-1H-1,2,4-triazol-3-yl)-2-methoxyphenyl)amino)-N-methylpyridazine-3-carboxamide (CPD-003)

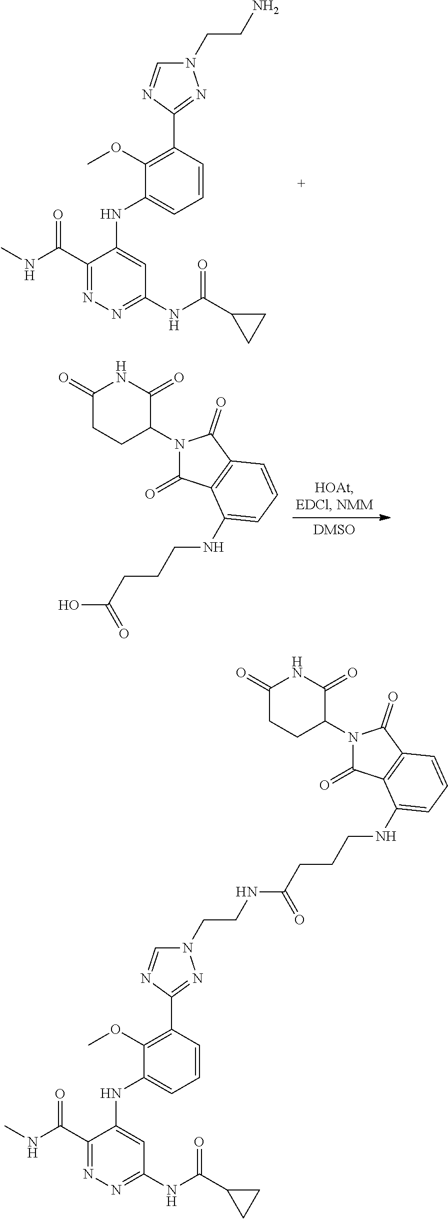 [SMILES: CNC(=O)c1nnc(NC(=O)C2CC2)cc1Nc1cccc(-c2ncn(CCN)n2)c1OC ; O=C(O)CCCNc1cccc2c1C(=O)N(C1CCC(=O)NC1=O)C2=O ; CNC(=O)c1nnc(NC(=O)C2CC2)cc1Nc1cccc(-c2ncn(CCNC(=O)CCCNc3cccc4c3C(=O)N(C3CCC(=O)NC3=O)C4=O)n2)c1OC]

CPD-003 was synthesized following the same procedure for preparing CPD-042 (2.74 mg, yield: 31%). MS (ESI) m/z=793.7 [M+H]⁺.

### Example 47: 6-(Cyclopropanecarboxamido)-4-((3-(1-(2-(5-((2-(2,6-dioxopiperidin-3-yl)-1,3-dioxoisoindolin-4-yl)amino)pentanamido)ethyl)-1H-1,2,4-triazol-3-yl)-2-methoxyphenyl)amino)-N-methylpyridazine-3-carboxamide (CPD-004)

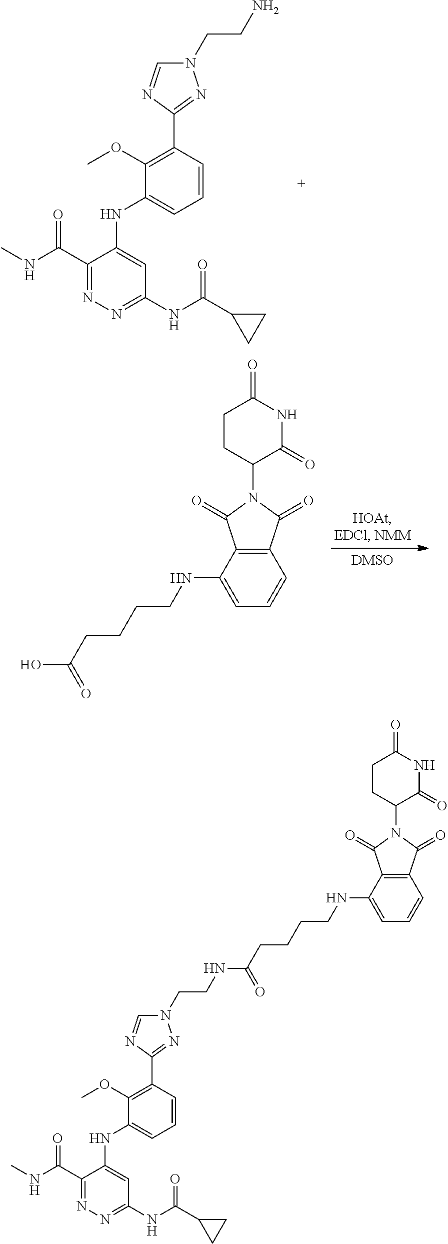

CPD-004 was synthesized following the same procedure for preparing CPD-042 (2.13 mg, yield: 24%). MS (ESI) m/z=807.7 [M+H]⁺.

### Example 48: 6-(Cyclopropanecarboxamido)-4-((3-(1-(2-(6-((2-(2,6-dioxopiperidin-3-yl)-1,3-dioxoisoindolin-4-yl)amino)hexanamido)ethyl)-1H-1,2,4-triazol-3-yl)-2-methoxyphenyl)amino)-N-methylpyridazine-3-carboxamide (CPD-005)

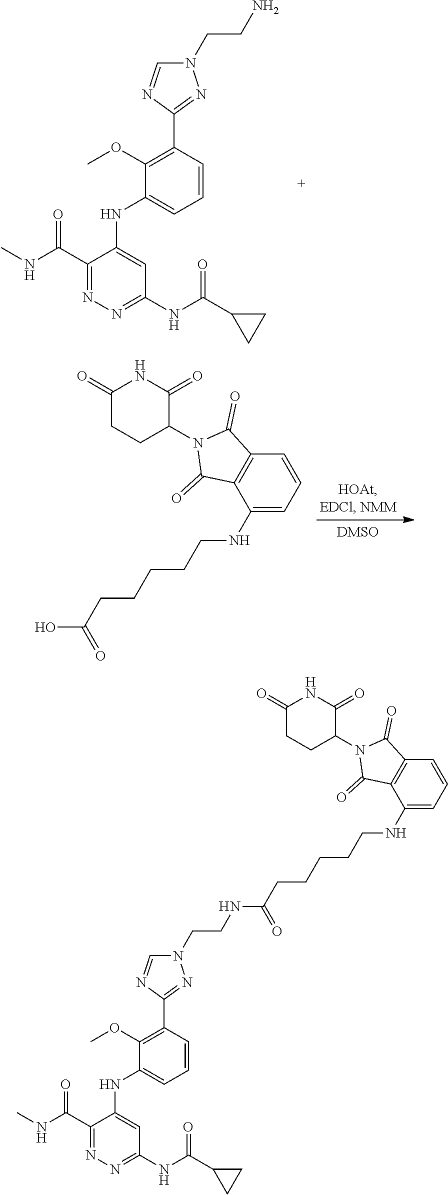 [SMILES: CNC(=O)c1nnc(NC(=O)C2CC2)cc1Nc1cccc(-c2ncn(CCN)n2)c1OC ; O=C(O)CCCCCNc1cccc2c1C(=O)N(C1CCC(=O)NC1=O)C2=O ; CNC(=O)c1nnc(NC(=O)C2CC2)cc1Nc1cccc(-c2ncn(CCNC(=O)CCCCCNc3cccc4c3C(=O)N(C3CCC(=O)NC3=O)C4=O)n2)c1OC]

CPD-005 was synthesized following the same procedure for preparing CPD-042 (2.44 mg, yield: 27%). MS (ESI) m/z=821.8 [M+H]⁺.

### Example 49: 6-(Cyclopropanecarboxamido)-4-((3-(1-(2-(7-((2-(2,6-dioxopiperidin-3-yl)-1,3-dioxoisoindolin-4-yl)amino)heptanamido)ethyl)-1H-1,2,4-triazol-3-yl)-2-methoxyphenyl)amino)-N-methylpyridazine-3-carboxamide (CPD-006)

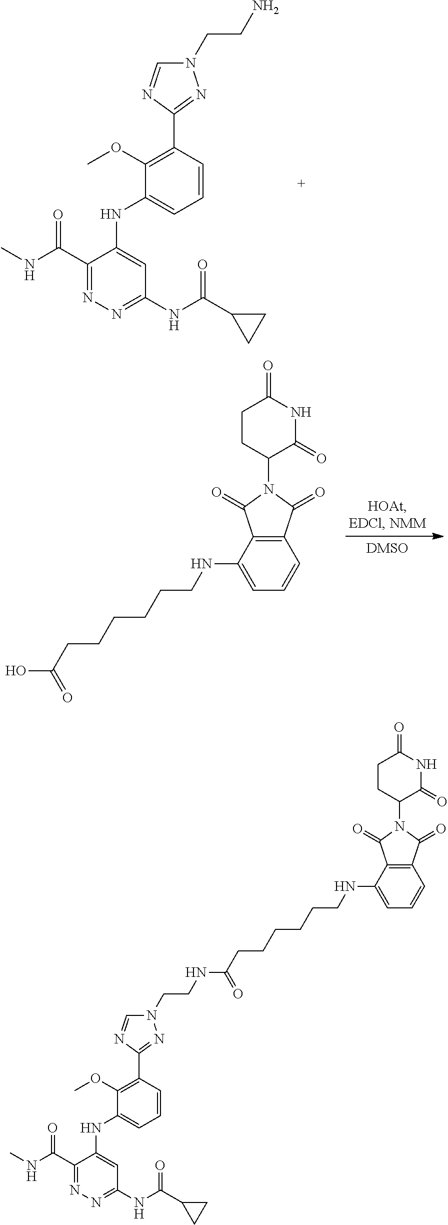

CPD-006 was synthesized following the same procedure for preparing CPD-042 (2.82 mg, yield: 31%). MS (ESI) m/z=835.8 [M+H]⁺.

### Example 50: 6-(Cyclopropanecarboxamido)-4-((3-(1-(2-(8-((2-(2,6-dioxopiperidin-3-yl)-1,3-dioxoisoindolin-4-yl)amino)octanamido)ethyl)-1H-1,2,4-triazol-3-yl)-2-methoxyphenyl)amino)-N-methylpyridazine-3-carboxamide (CPD-007)

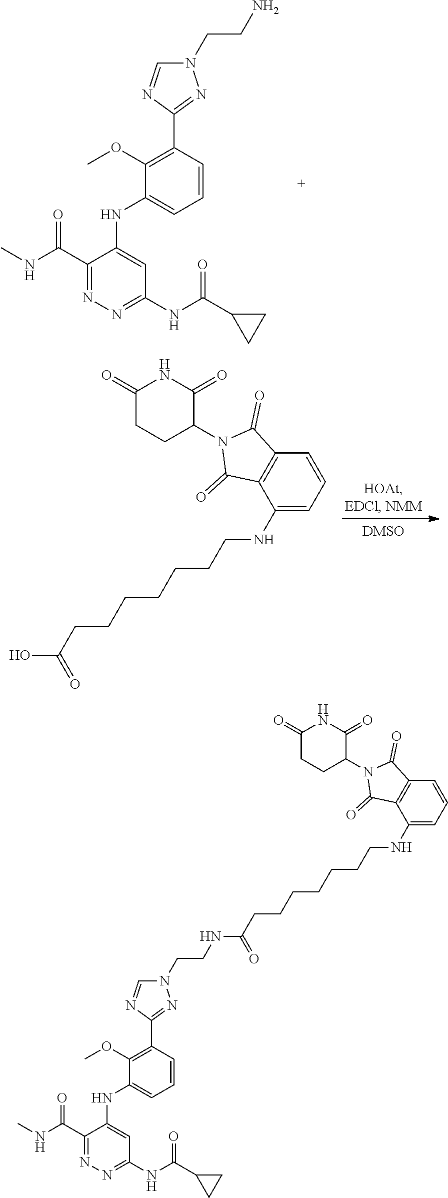

CPD-007 was synthesized following the same procedure for preparing CPD-042 (2.91 mg, yield: 31%). MS (ESI) m/z=849.8 [M+H]⁺.

### Example 51: 6-(Cyclopropanecarboxamido)-4-((3-(1-(2-(3-(2-((2-(2,6-dioxopiperidin-3-yl)-1,3-dioxoisoindolin-4-yl)amino)ethoxy)propanamido)ethyl)-1H-1,2,4-triazol-3-yl)-2-methoxyphenyl)amino)-N-methylpyridazine-3-carboxamide (CPD-008)

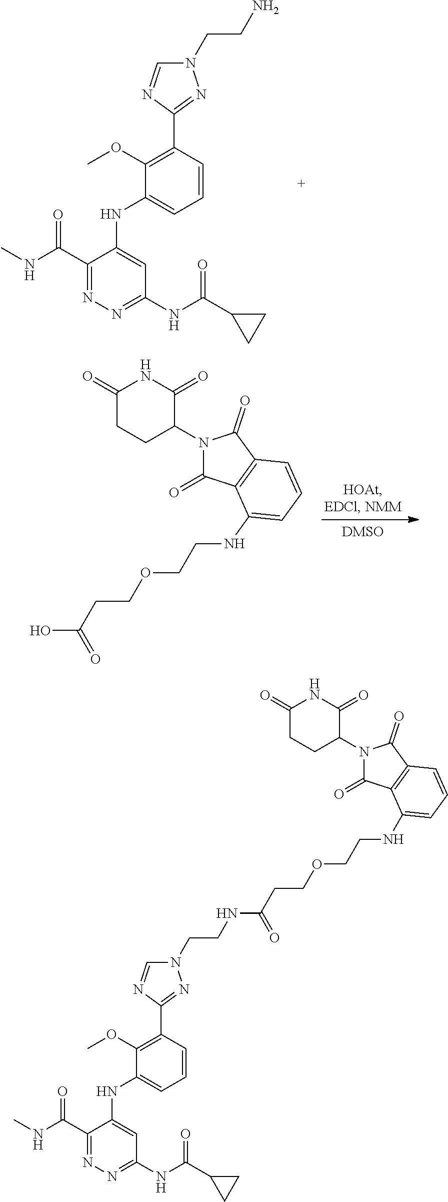 [SMILES: CNC(=O)c1nnc(NC(=O)C2CC2)cc1Nc1cccc(-c2ncn(CCN)n2)c1OC ; O=C(O)CCOCCNc1cccc2c1C(=O)N(C1CCC(=O)NC1=O)C2=O ; CNC(=O)c1nnc(NC(=O)C2CC2)cc1Nc1cccc(-c2ncn(CCNC(=O)CCOCCNc3cccc4c3C(=O)N(C3CCC(=O)NC3=O)C4=O)n2)c1OC]

CPD-008 was synthesized following the same procedure for preparing CPD-042 (2.14 g, yield: 24%). MS (ESI) m/z=823.7 [M+H]⁺.

### Example 52: 6-(Cyclopropanecarboxamido)-4-((3-(1-(2-(3-(2-(2-((2-(2,6-dioxopiperidin-3-yl)-1,3-dioxoisoindolin-4-yl)amino)ethoxy)ethoxy)propanamido)ethyl)-1H-1,2,4-triazol-3-yl)-2-methoxyphenyl)amino)-N-methylpyridazine-3-carboxamide (CPD-009)

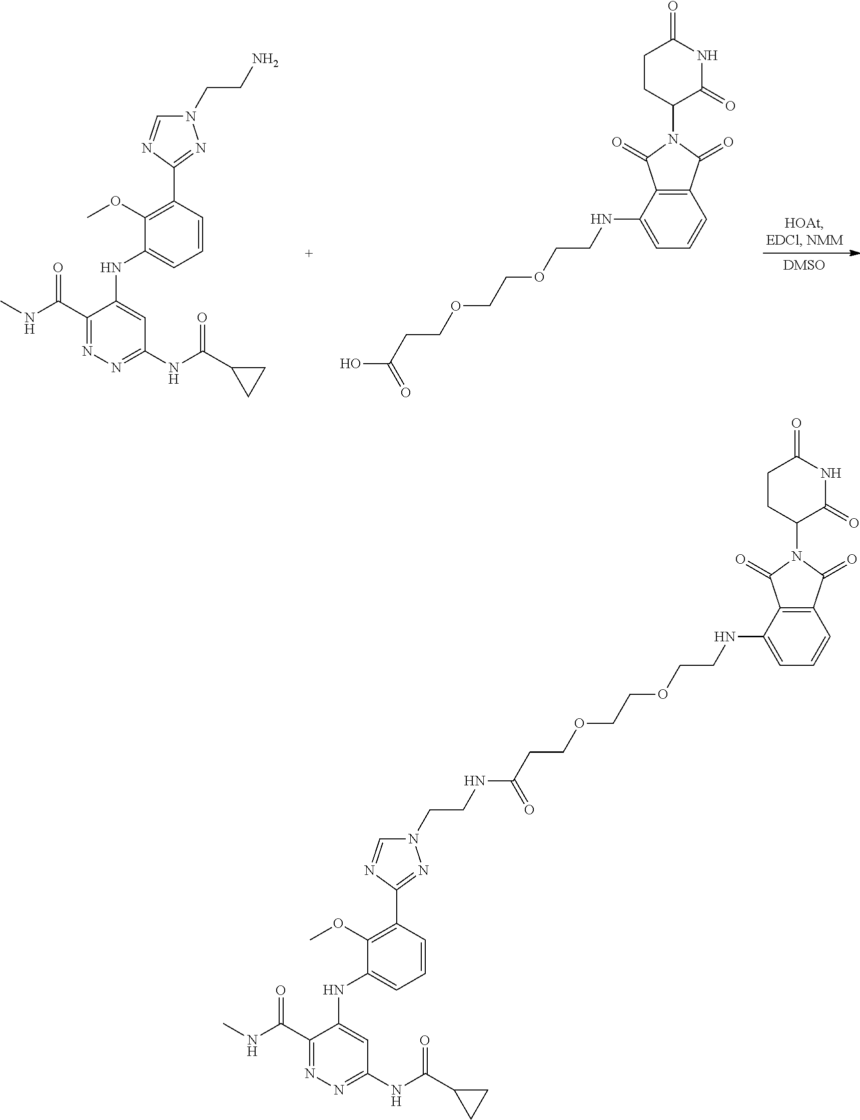 [SMILES: CNC(=O)c1nnc(NC(=O)C2CC2)cc1Nc1cccc(-c2ncn(CCN)n2)c1OC ; O=C(O)CCOCCOCCNc1cccc2c1C(=O)N(C1CCC(=O)NC1=O)C2=O ; CNC(=O)c1nnc(NC(=O)C2CC2)cc1Nc1cccc(-c2ncn(CCNC(=O)CCOCCOCCNc3cccc4c3C(=O)N(C3CCC(=O)NC3=O)C4=O)n2)c1OC]

CPD-009 was synthesized following the same procedure for preparing CPD-042 (2.39 mg, yield: 25%). MS (ESI) m/z=867.7 [M+H]⁺.

### Example 53: 6-(Cyclopropanecarboxamido)-4-((3-(1-(1-((2-(2,6-dioxopiperidin-3-yl)-1,3-dioxoisoindolin-4-yl)amino)-12-oxo-3,6,9-trioxa-13-azapentadecan-15-yl)-1H-1,2,4-triazol-3-yl)-2-methoxyphenyl)amino)-N-methylpyridazine-3-carboxamide (CPD-010)

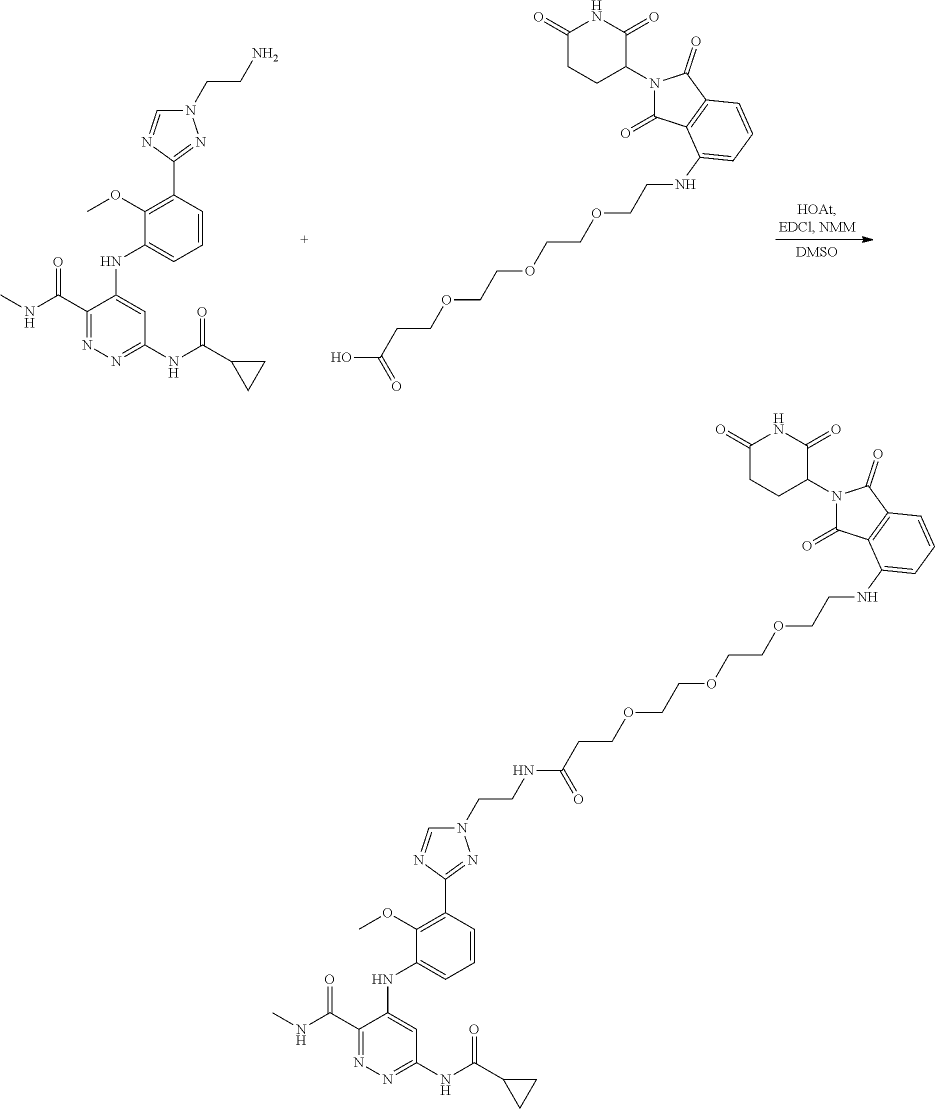 [SMILES: CNC(=O)c1nnc(NC(=O)C2CC2)cc1Nc1cccc(-c2ncn(CCN)n2)c1OC ; O=C(O)CCOCCOCCOCCNc1cccc2c1C(=O)N(C1CCC(=O)NC1=O)C2=O ; CNC(=O)c1nnc(NC(=O)C2CC2)cc1Nc1cccc(-c2ncn(CCNC(=O)CCOCCOCCOCCNc3cccc4c3C(=O)N(C3CCC(=O)NC3=O)C4=O)n2)c1OC]

CPD-010 was synthesized following the same procedure for preparing CPD-042 (2.63 mg, yield: 26%). MS (ESI) m/z=911.8 [M+H]⁺.

### Example 54: 6-(Cyclopropanecarboxamido)-4-((3-(1-(1-((2-(2,6-dioxopiperidin-3-yl)-1,3-dioxoisoindolin-4-yl)amino)-15-oxo-3,6,9,12-tetraoxa-16-azaoctadecan-18-yl)-1H-1,2,4-triazol-3-yl)-2-methoxyphenyl)amino)-N-methylpyridazine-3-carboxamide (CPD-011)

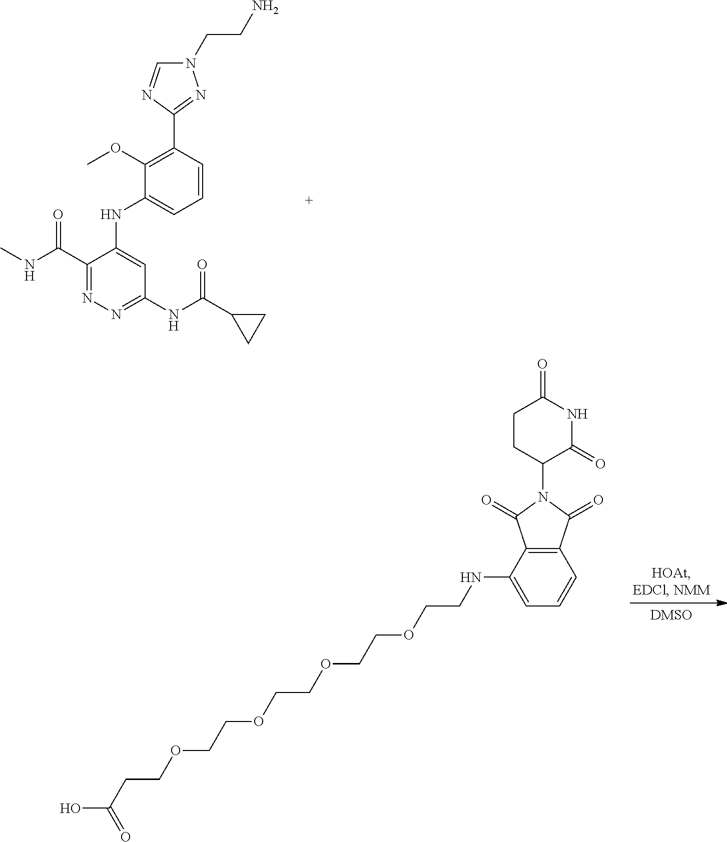 [SMILES: CNC(=O)c1nnc(NC(=O)C2CC2)cc1Nc1cccc(-c2ncn(CCN)n2)c1OC ; O=C(O)CCOCCOCCOCCOCCNc1cccc2c1C(=O)N(C1CCC(=O)NC1=O)C2=O]

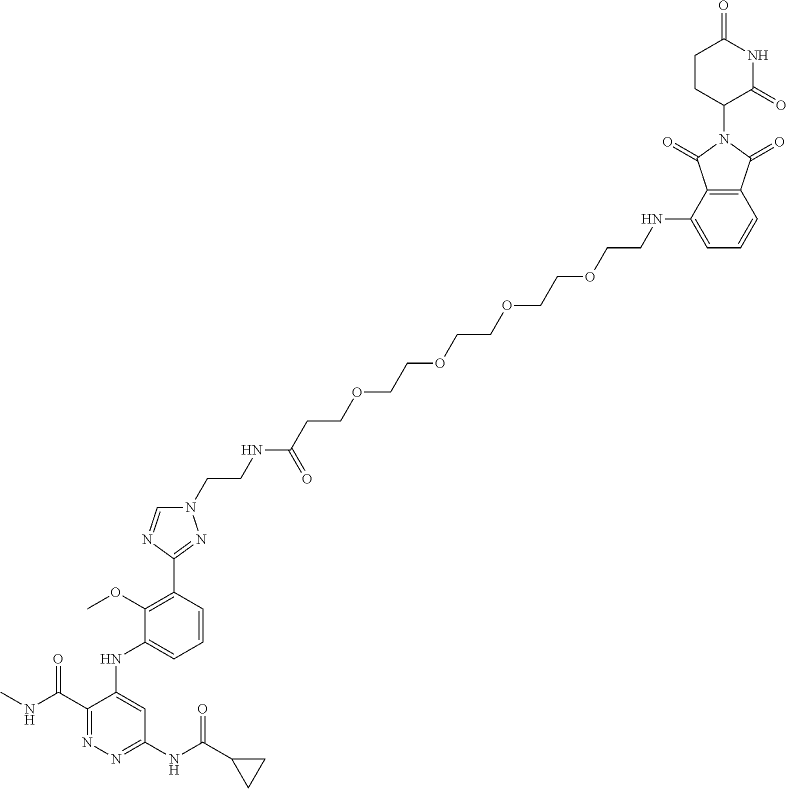 [SMILES: CNC(=O)c1nnc(NC(=O)C2CC2)cc1Nc1cccc(-c2ncn(CCNC(=O)CCOCCOCCOCCOCCNc3cccc4c3C(=O)N(C3CCC(=O)NC3=O)C4=O)n2)c1OC]

CPD-011 was synthesized following the same procedure for preparing CPD-042 (1.36 mg, yield: 13%). MS (ESI) m/z=955.8 [M+H]⁺.

### Example 55: 6-(Cyclopropanecarboxamido)-4-((3-(1-(1-((2-(2,6-dioxopiperidin-3-yl)-1,3-dioxoisoindolin-4-yl)amino)-18-oxo-3,6,9,12,15-pentaoxa-19-azahenicosan-2l-yl)-1H-1,2,4-triazol-3-yl)-2-methoxyphenyl)amino)-N-methylpyridazine-3-carboxamide (CPD-012)

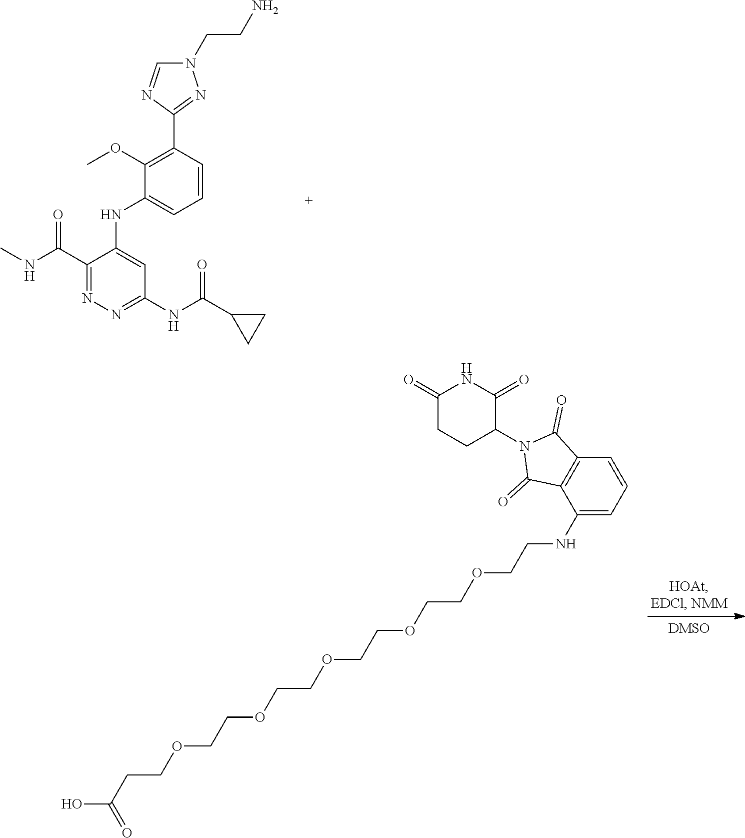 [SMILES: CNC(=O)c1nnc(NC(=O)C2CC2)cc1Nc1cccc(-c2ncn(CCN)n2)c1OC ; O=C(O)CCOCCOCCOCCOCCOCCNc1cccc2c1C(=O)N(C1CCC(=O)NC1=O)C2=O]

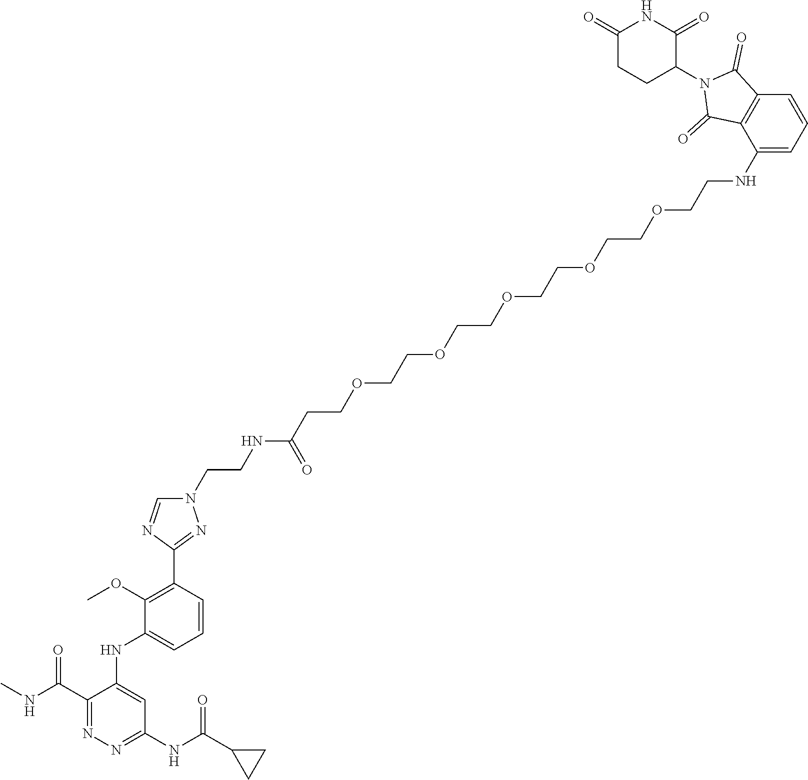 [SMILES: CNC(=O)c1nnc(NC(=O)C2CC2)cc1Nc1cccc(-c2ncn(CCNC(=O)CCOCCOCCOCCOCCOCCNc3cccc4c3C(=O)N(C3CCC(=O)NC3=O)C4=O)n2)c1OC]

CPD-012 was synthesized following the same procedure for preparing CPD-042 (2.53 mg, yield: 23%). MS (ESI) m/z=999.7 [M+H]⁺.

### Example 56: 6-(Cyclopropanecarboxamido)-4-((3-(1-(2-(2-((2-(2,6-dioxopiperidin-3-yl)-1,3-dioxoisoindolin-5-yl)amino)acetamido)ethyl)-1H-1,2,4-triazol-3-yl)-2-methoxyphenyl)amino)-N-methylpyridazine-3-carboxamide (CPD-013)

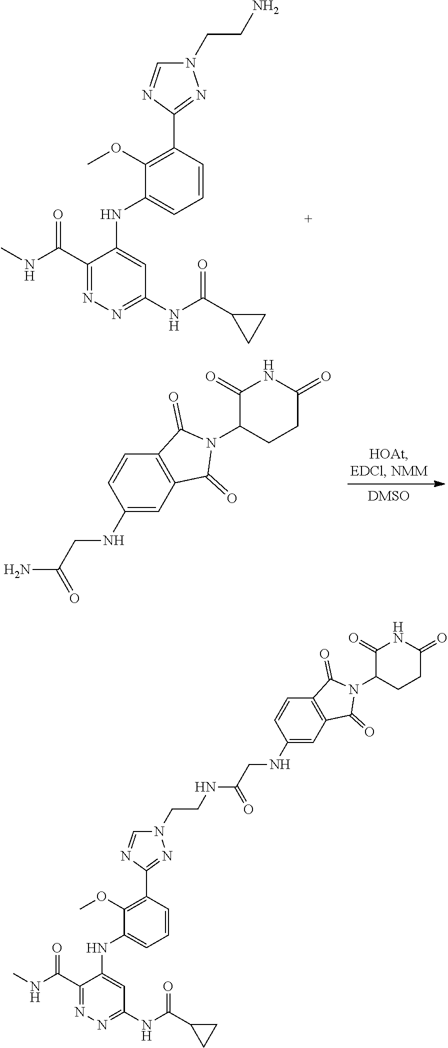

CPD-013 was synthesized following the same procedure for preparing CPD-042 (2.2 mg, yield: 43%). MS (ESI) m/z=765.6 [M+H]⁺.

### Example 57: 6-(Cyclopropanecarboxamido)-4-((3-(1-(2-(3-((2-(2,6-dioxopiperidin-3-yl)-1,3-dioxoisoindolin-5-yl)amino)propanamido)ethyl)-1H-1,2,4-triazol-3-yl)-2-methoxyphenyl)amino)-N-methylpyridazine-3-carboxamide (CPD-014)

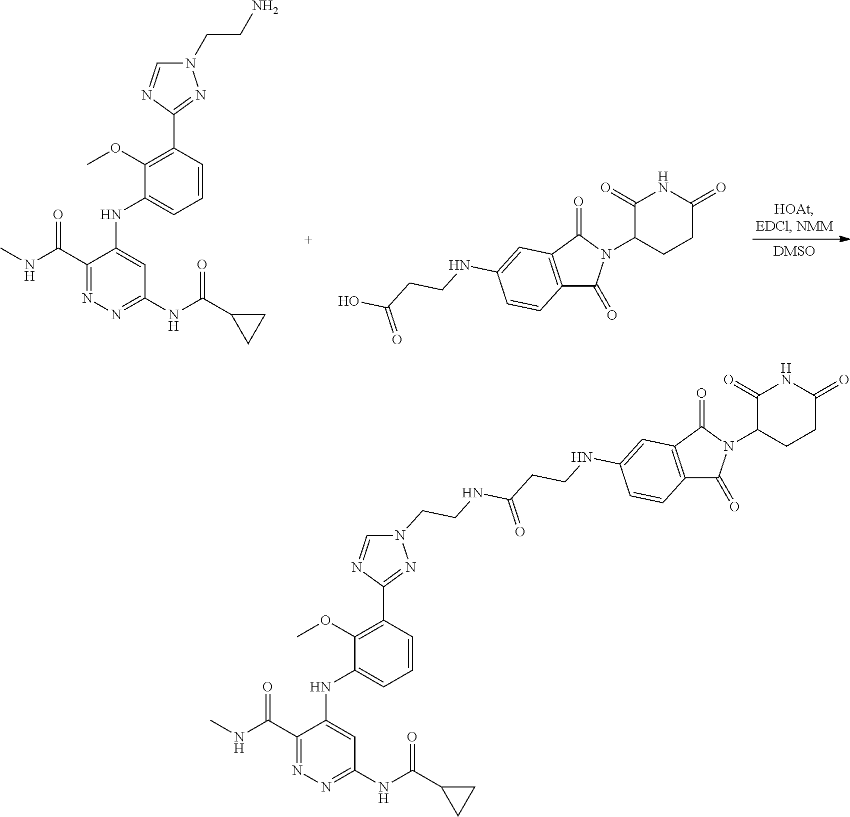 [SMILES: CNC(=O)c1nnc(NC(=O)C2CC2)cc1Nc1cccc(-c2ncn(CCN)n2)c1OC ; O=C(O)CCNc1ccc2c(c1)C(=O)N(C1CCC(=O)NC1=O)C2=O ; CNC(=O)c1nnc(NC(=O)C2CC2)cc1Nc1cccc(-c2ncn(CCNC(=O)CCNc3ccc4c(c3)C(=O)N(C3CCC(=O)NC3=O)C4=O)n2)c1OC]

CPD-014 was synthesized following the same procedure for preparing CPD-042 (2.29 mg, yield: 44%). MS (ESI) m/z=779.7 [M+H]⁺.

### Example 58: 6-(Cyclopropanecarboxamido)-4-((3-(1-(2-(4-((2-(2,6-dioxopiperidin-3-yl)-1,3-dioxoisoindolin-5-yl)amino)butanamido)ethyl)-1H-1,2,4-triazol-3-yl)-2-methoxyphenyl)amino)-N-methylpyridazine-3-carboxamide (CPD-015)

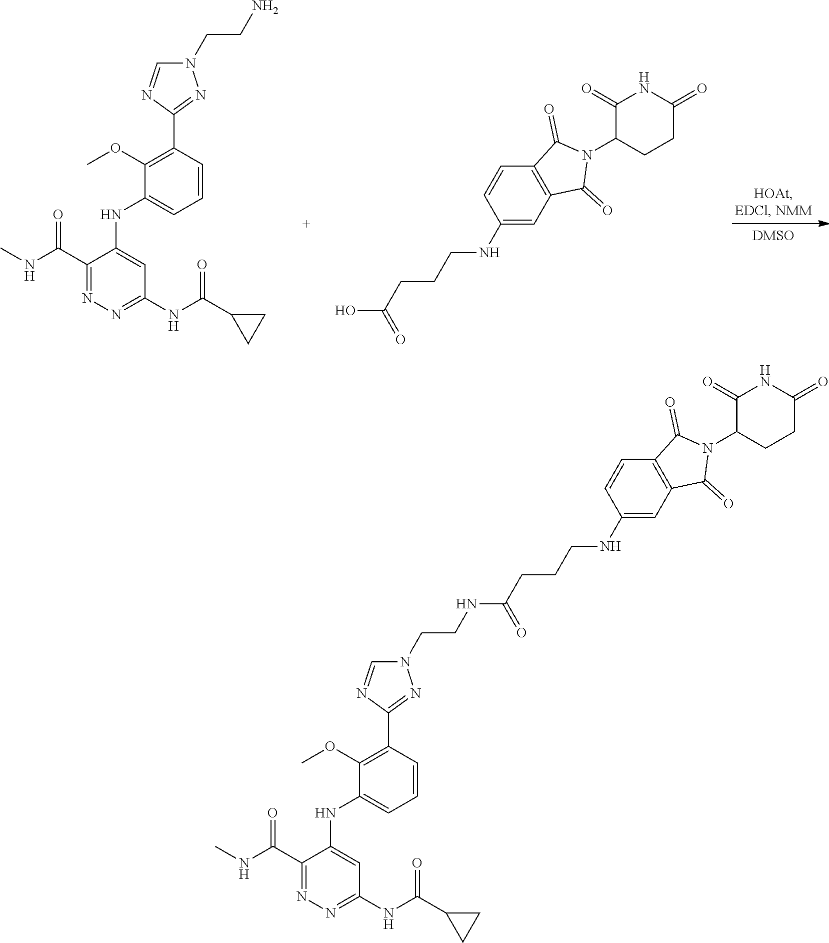 [SMILES: CNC(=O)c1nnc(NC(=O)C2CC2)cc1Nc1cccc(-c2ncn(CCN)n2)c1OC ; O=C(O)CCCNc1ccc2c(c1)C(=O)N(C1CCC(=O)NC1=O)C2=O ; CNC(=O)c1nnc(NC(=O)C2CC2)cc1Nc1cccc(-c2ncn(CCNC(=O)CCCNc3ccc4c(c3)C(=O)N(C3CCC(=O)NC3=O)C4=O)n2)c1OC]

CPD-015 was synthesized following the same procedure for preparing CPD-042 (2.05 mg, yield: 39%). MS (ESI) m/z=793.7 [M+H]⁺.

### Example 59: 6-(Cyclopropanecarboxamido)-4-((3-(1-(2-(5-((2-(2,6-dioxopiperidin-3-yl)-1,3-dioxoisoindolin-5-yl)amino)pentanamido)ethyl)-1H-1,2,4-triazol-3-yl)-2-methoxyphenyl)amino)-N-methylpyridazine-3-carboxamide (CPD-016)

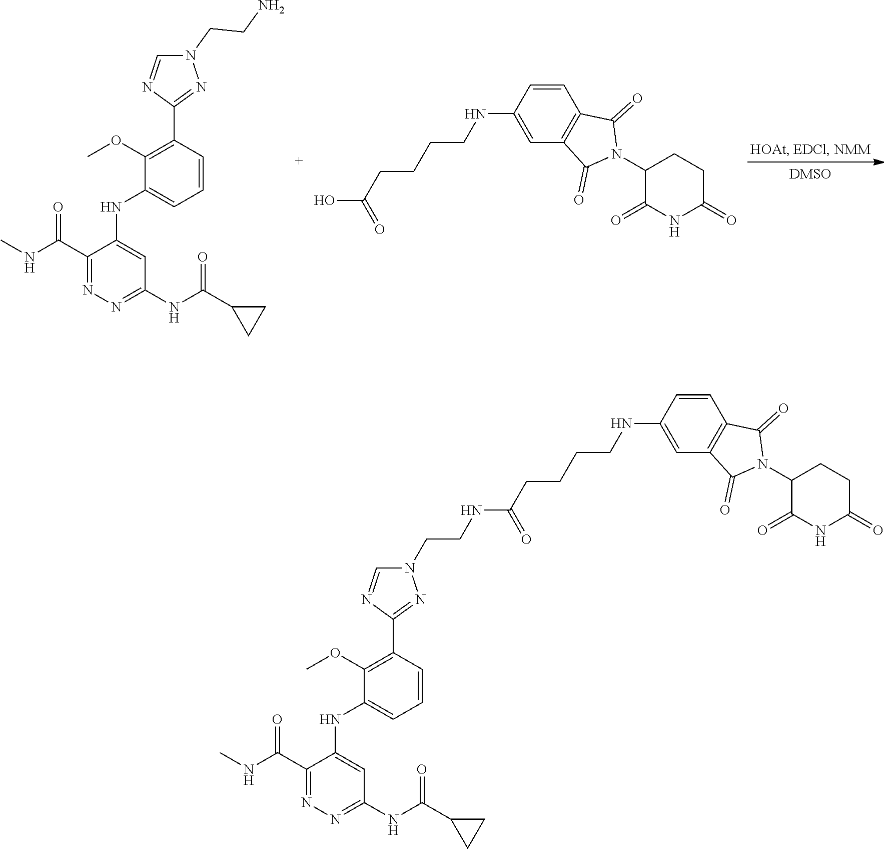 [SMILES: CNC(=O)c1nnc(NC(=O)C2CC2)cc1Nc1cccc(-c2ncn(CCN)n2)c1OC ; "+" ; O=C(O)CCCCNc1ccc2c(c1)C(=O)N(C1CCC(=O)NC1=O)C2=O ; CNC(=O)c1nnc(NC(=O)C2CC2)cc1Nc1cccc(-c2ncn(CCNC(=O)CCCCNc3ccc4c(c3)C(=O)N(C3CCC(=O)NC3=O)C4=O)n2)c1OC]

CPD-016 was synthesized following the same procedure for preparing CPD-042 (2.39 mg, yield: 44%). MS (ESI) m/z=807.7 [M+H]⁺.

### Example 60: 6-(Cyclopropanecarboxamido)-4-((3-(1-(2-(7-((2-(2,6-dioxopiperidin-3-yl)-1,3-dioxoisoindolin-5-yl)amino)heptanamido)ethyl)-1H-1,2,4-triazol-3-yl)-2-methoxyphenyl)amino)-N-methylpyridazine-3-carboxamide (CPD-017)

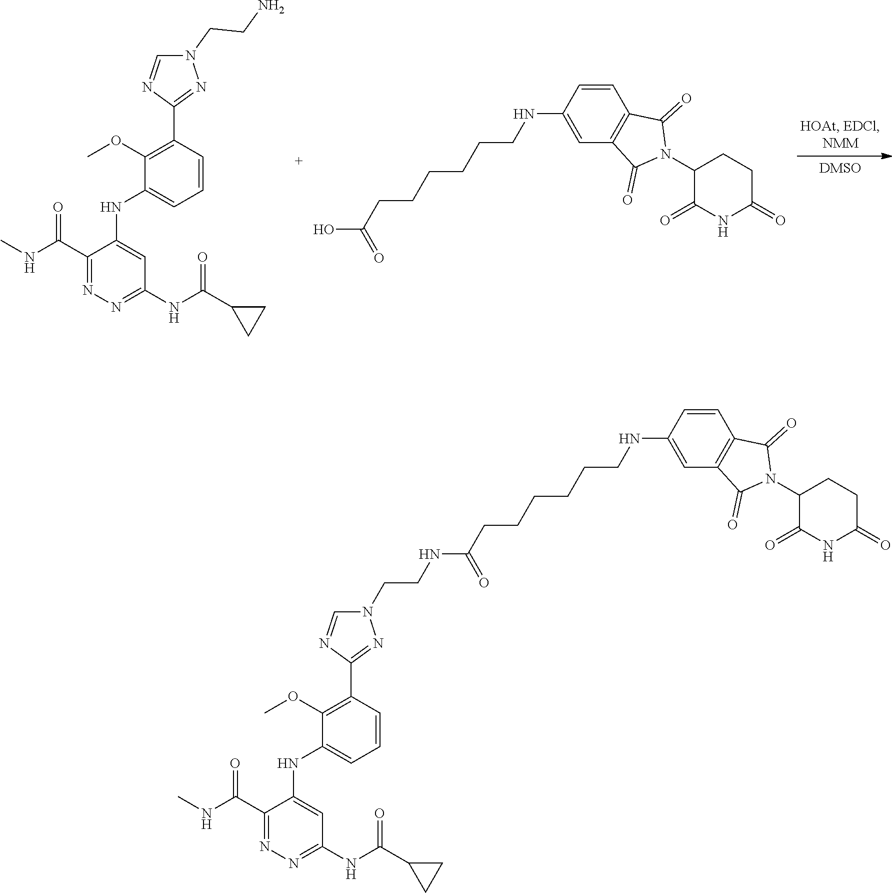 [SMILES: CNC(=O)c1nnc(NC(=O)C2CC2)cc1Nc1cccc(-c2ncn(CCN)n2)c1OC ; "+" ; O=C(O)CCCCCCNc1ccc2c(c1)C(=O)N(C1CCC(=O)NC1=O)C2=O ; CNC(=O)c1nnc(NC(=O)C2CC2)cc1Nc1cccc(-c2ncn(CCNC(=O)CCCCCCNc3ccc4c(c3)C(=O)N(C3CCC(=O)NC3=O)C4=O)n2)c1OC]

CPD-017 was synthesized following the same procedure for preparing CPD-042 (2.13 mg, yield: 38%). MS (ESI) m/z=835.8 [M+H]⁺.

### Example 61: 6-(Cyclopropanecarboxamido)-4-((3-(1-(2-(3-(2-((2-(2,6-dioxopiperidin-3-yl)-1,3-dioxoisoindolin-5-yl)amino)ethoxy)propanamido)ethyl)-1H-1,2,4-triazol-3-yl)-2-methoxyphenyl)amino)-N-methylpyridazine-3-carboxamide (CPD-018)

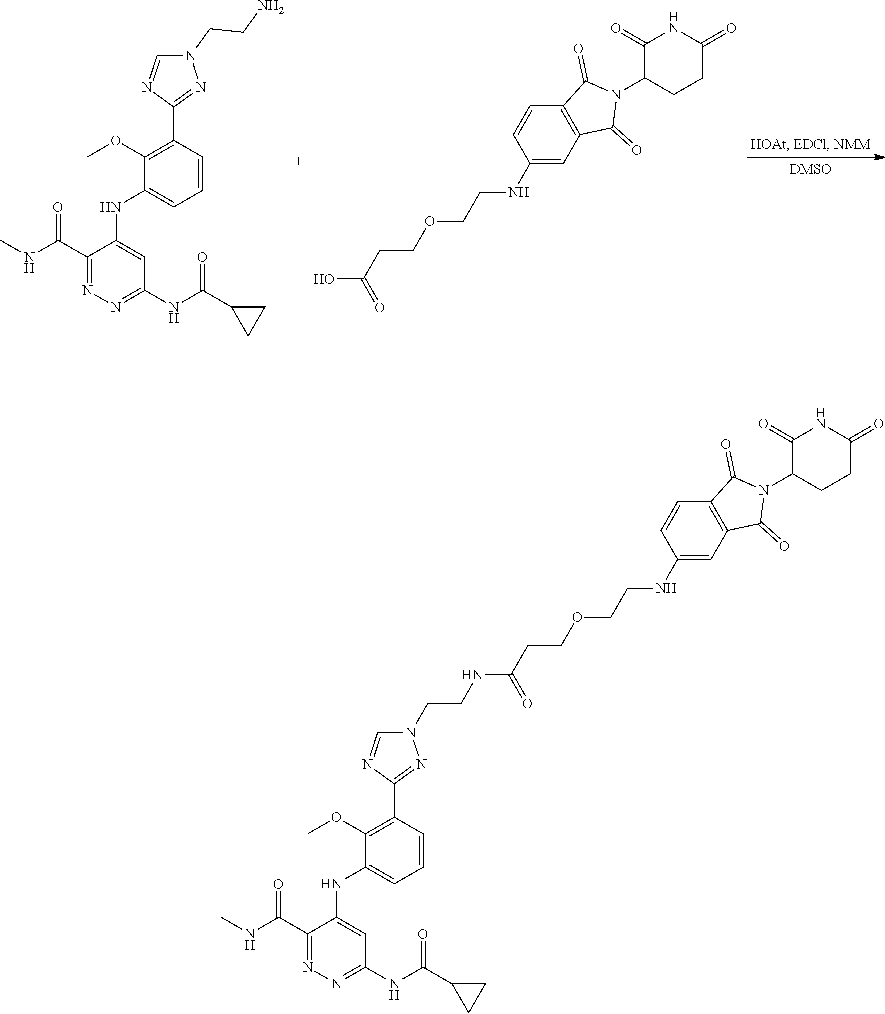 [SMILES: CNC(=O)c1nnc(NC(=O)C2CC2)cc1Nc1cccc(-c2ncn(CCN)n2)c1OC ; "+" ; O=C(O)CCOCCNc1ccc2c(c1)C(=O)N(C1CCC(=O)NC1=O)C2=O ; CNC(=O)c1nnc(NC(=O)C2CC2)cc1Nc1cccc(-c2ncn(CCNC(=O)CCOCCNc3ccc4c(c3)C(=O)N(C3CCC(=O)NC3=O)C4=O)n2)c1OC]

CPD-018 was synthesized following the same procedure for preparing CPD-042 (2.15 mg, yield: 39%). MS (ESI) m/z=823.7 [M+H]⁺.

### Example 62: 6-(Cyclopropanecarboxamido)-4-((3-(1-(2-(3-(2-(2-((2-(2,6-dioxopiperidin-3-yl)-1,3-dioxoisoindolin-5-yl)amino)ethoxy)ethoxy)propanamido)ethyl)-1H-1,2,4-triazol-3-yl)-2-methoxyphenyl)amino)-N-methylpyridazine-3-carboxamide (CPD-019)

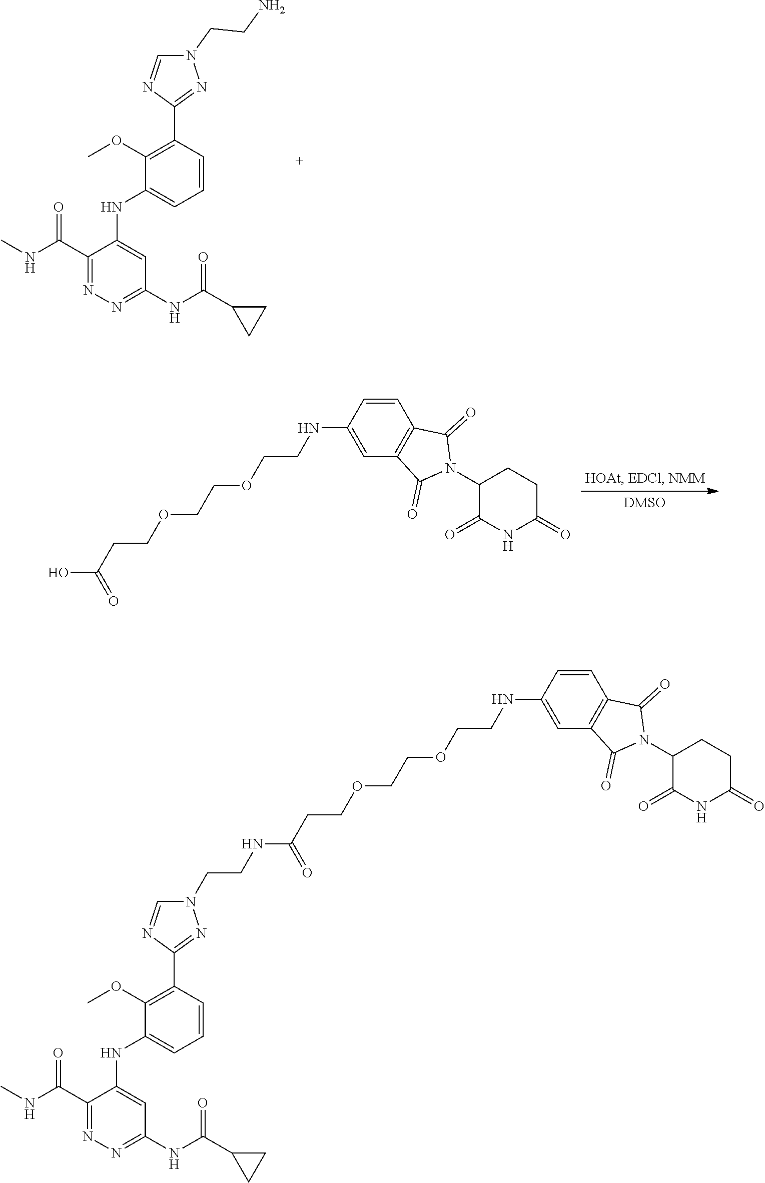 [SMILES: CNC(=O)c1nnc(NC(=O)C2CC2)cc1Nc1cccc(-c2ncn(CCN)n2)c1OC ; "+" ; O=C(O)CCOCCOCCNc1ccc2c(c1)C(=O)N(C1CCC(=O)NC1=O)C2=O ; CNC(=O)c1nnc(NC(=O)C2CC2)cc1Nc1cccc(-c2ncn(CCNC(=O)CCOCCOCCNc3ccc4c(c3)C(=O)N(C3CCC(=O)NC3=O)C4=O)n2)c1OC]

CPD-019 was synthesized following the same procedure for preparing CPD-042 (2.5 mg, yield: 43%). MS (ESI) m/z=867.7 [M+H]⁺.

### Example 63: 6-(Cyclopropanecarboxamido)-4-((3-(1-(1-((2-(2,6-dioxopiperidin-3-yl)-1,3-dioxoisoindolin-5-yl)amino)-12-oxo-3,6,9-trioxa-13-azapentadecan-15-yl)-1H-1,2,4-triazol-3-yl)-2-methoxyphenyl)amino)-N-methylpyridazine-3-carboxamide (CPD-020)

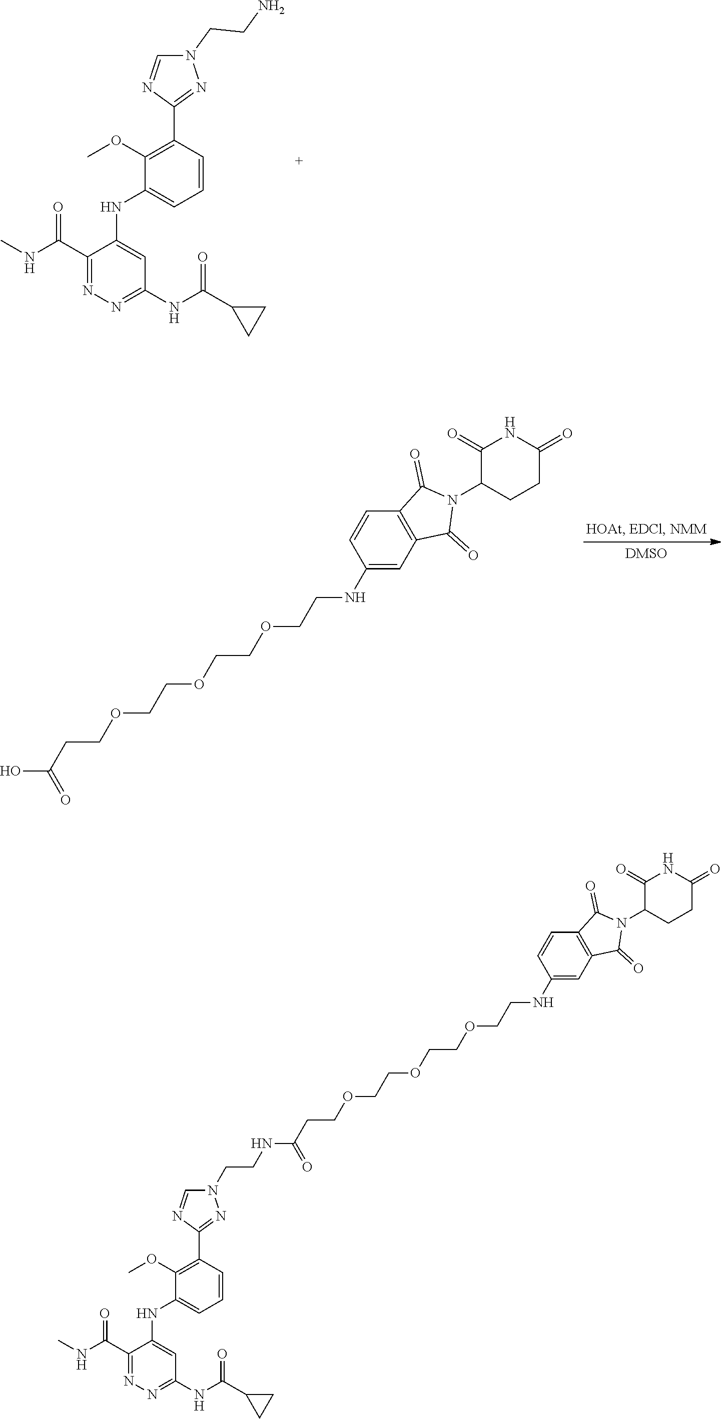 [SMILES: CNC(=O)c1nnc(NC(=O)C2CC2)cc1Nc1cccc(-c2ncn(CCN)n2)c1OC ; "+" ; O=C(O)CCOCCOCCOCCNc1ccc2c(c1)C(=O)N(C1CCC(=O)NC1=O)C2=O ; CNC(=O)c1nnc(NC(=O)C2CC2)cc1Nc1cccc(-c2ncn(CCNC(=O)CCOCCOCCOCCNc3ccc4c(c3)C(=O)N(C3CCC(=O)NC3=O)C4=O)n2)c1OC]

CPD-020 was synthesized following the same procedure for preparing CPD-042 (3.23 mg, yield: 53%). MS (ESI) m/z=911.8 [M+H]⁺.

### Example 64: 6-(Cyclopropanecarboxamido)-4-((3-(1-(1-((2-(2,6-dioxopiperidin-3-yl)-1,3-dioxoisoindolin-5-yl)amino)-15-oxo-3,6,9,12-tetraoxa-16-azaoctadecan-18-yl)-1H-1,2,4-triazol-3-yl)-2-methoxyphenyl)amino)-N-methylpyridazine-3-carboxamide (CPD-021)

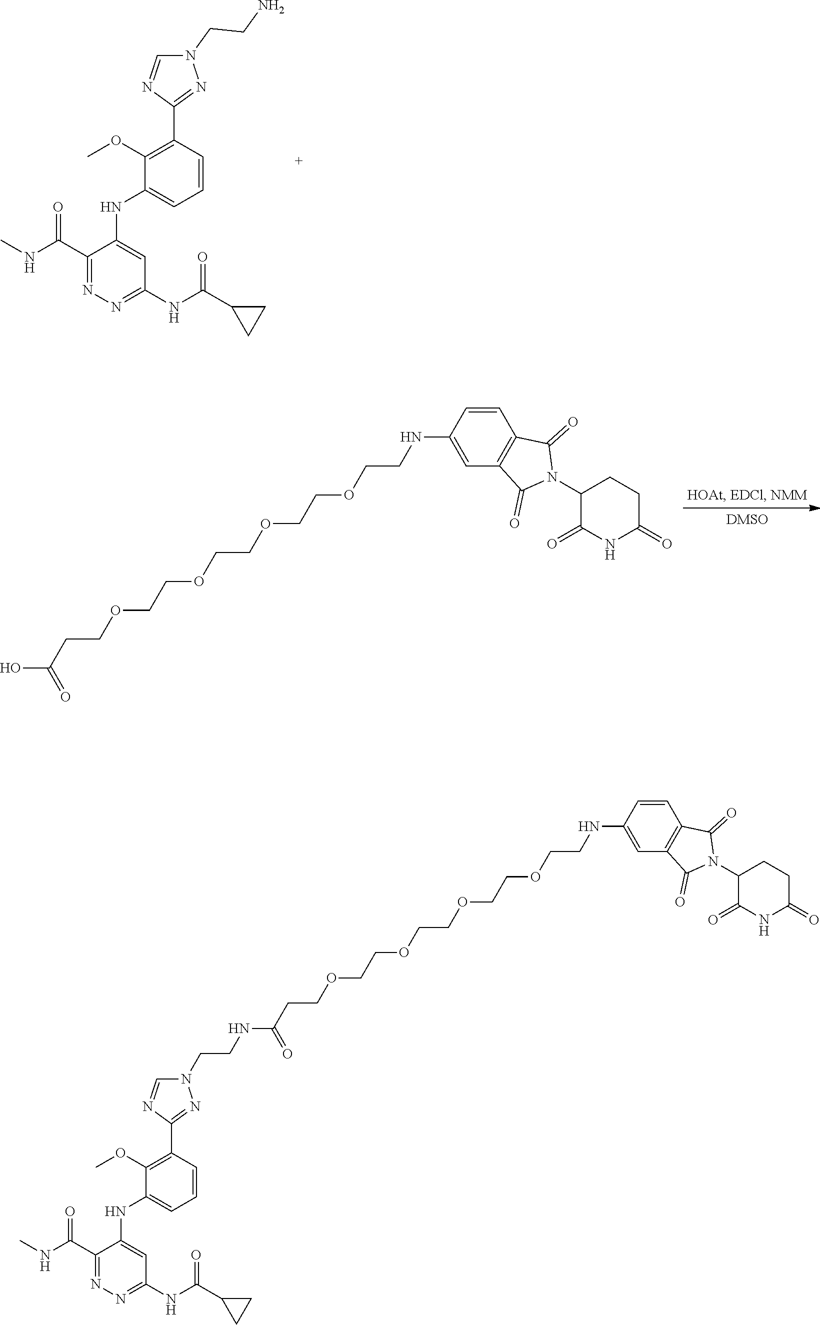 [SMILES: CNC(=O)c1nnc(NC(=O)C2CC2)cc1Nc1cccc(-c2ncn(CCN)n2)c1OC ; "+" ; O=C(O)CCOCCOCCOCCOCCNc1ccc2c(c1)C(=O)N(C1CCC(=O)NC1=O)C2=O ; CNC(=O)c1nnc(NC(=O)C2CC2)cc1Nc1cccc(-c2ncn(CCNC(=O)CCOCCOCCOCCOCCNc3ccc4c(c3)C(=O)N(C3CCC(=O)NC3=O)C4=O)n2)c1OC]

CPD-021 was synthesized following the same procedure for preparing CPD-042 (3.24 mg, yield: 51%). MS (ESI) m/z=955.8 [M+H]⁺.

### Example 65: 6-(Cyclopropanecarboxamido)-4-((3-(1-(1-((2-(2,6-dioxopiperidin-3-yl)-1,3-dioxoisoindolin-5-yl)amino)-18-oxo-3,6,9,12,15-pentaoxa-19-azahenicosan-21-yl)-1H-1,2,4-triazol-3-yl)-2-methoxyphenyl)amino)-N-methylpyridazine-3-carboxamide (CPD-022)

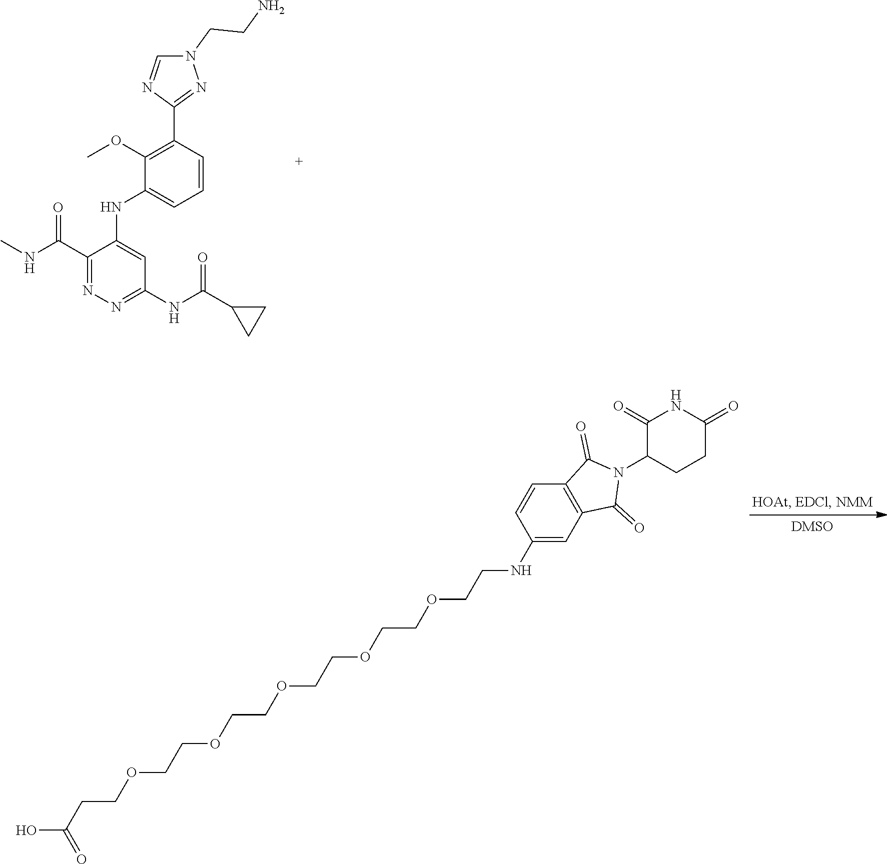 [SMILES: CNC(=O)c1nnc(NC(=O)C2CC2)cc1Nc1cccc(-c2ncn(CCN)n2)c1OC ; "+" ; O=C(O)CCOCCOCCOCCOCCOCCNc1ccc2c(c1)C(=O)N(C1CCC(=O)NC1=O)C2=O]

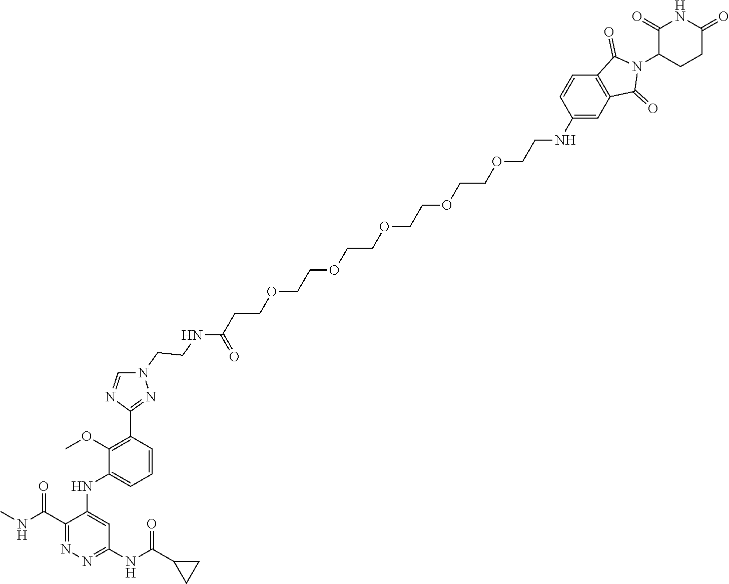 [SMILES: CNC(=O)c1nnc(NC(=O)C2CC2)cc1Nc1cccc(-c2ncn(CCNC(=O)CCOCCOCCOCCOCCOCCNc3ccc4c(c3)C(=O)N(C3CCC(=O)NC3=O)C4=O)n2)c1OC]

CPD-022 was synthesized following the same procedure for preparing CPD-042 (3.01 mg, yield: 45%). MS (ESI) m/z=999.7 [M+H]⁺.

### Example 66: 6-(Cyclopropanecarboxamido)-4-((3-(1-(2-(6-((2-(2,6-dioxopiperidin-3-yl)-1,3-dioxoisoindolin-5-yl)amino)hexanamido)ethyl)-1H-1,2,4-triazol-3-yl)-2-methoxyphenyl)amino)-N-methylpyridazine-3-carboxamide (CPD-023)

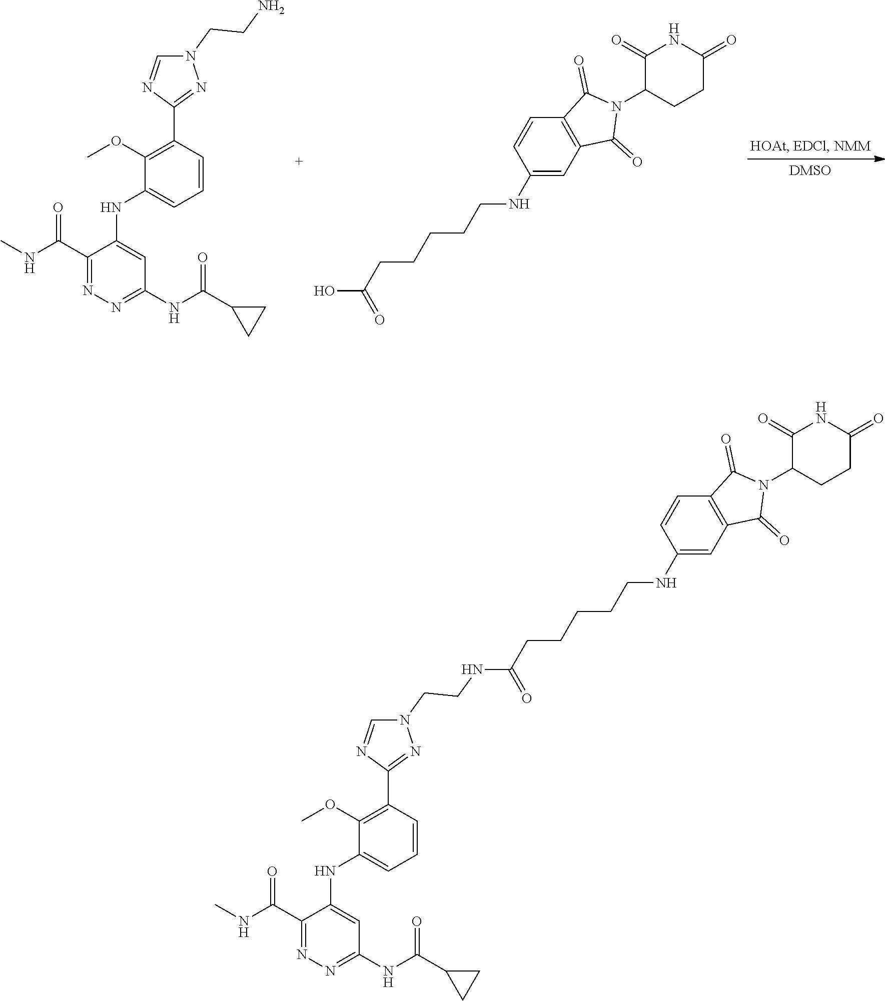 [SMILES: CNC(=O)c1nnc(NC(=O)C2CC2)cc1Nc1cccc(-c2ncn(CCN)n2)c1OC ; "+" ; O=C(O)CCCCCNc1ccc2c(c1)C(=O)N(C1CCC(=O)NC1=O)C2=O ; CNC(=O)c1nnc(NC(=O)C2CC2)cc1Nc1cccc(-c2ncn(CCNC(=O)CCCCCNc3ccc4c(c3)C(=O)N(C3CCC(=O)NC3=O)C4=O)n2)c1OC]

CPD-023 was synthesized following the same procedure for preparing CPD-042 (3.3 mg, yield: 60%). MS (ESI) m/z=821.7 [M+H]⁺.

### Example 67: 6-(Cyclopropanecarboxamido)-4-((3-(1-(2-(8-((2-(2,6-dioxopiperidin-3-yl)-1,3-dioxoisoindolin-5-yl)amino)octanamido)ethyl)-1H-1,2,4-triazol-3-yl)-2-methoxyphenyl)amino)-N-methylpyridazine-3-carboxamide (CPD-024)

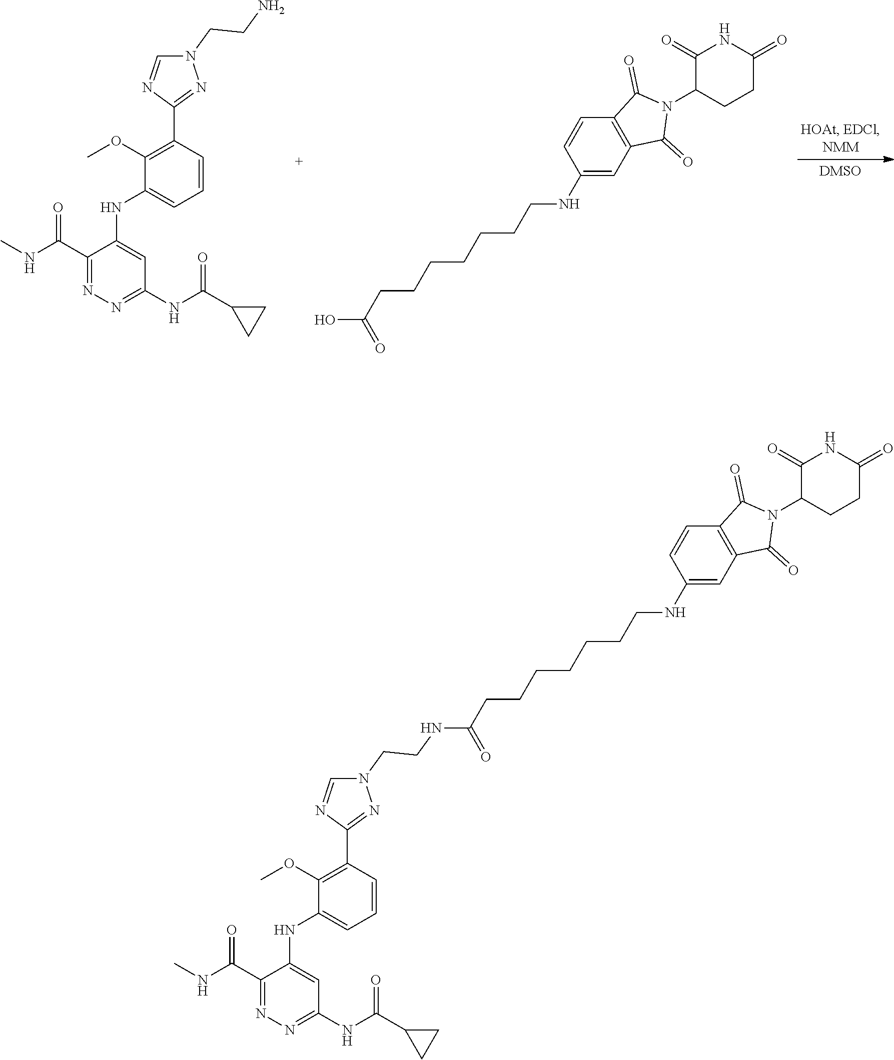 [SMILES: CNC(=O)c1nnc(NC(=O)C2CC2)cc1Nc1cccc(-c2ncn(CCN)n2)c1OC ; "+" ; O=C(O)CCCCCCCNc1ccc2c(c1)C(=O)N(C1CCC(=O)NC1=O)C2=O ; CNC(=O)c1nnc(NC(=O)C2CC2)cc1Nc1cccc(-c2ncn(CCNC(=O)CCCCCCCNc3ccc4c(c3)C(=O)N(C3CCC(=O)NC3=O)C4=O)n2)c1OC]

CPD-024 was synthesized following the same procedure for preparing CPD-042 (2.26 mg, yield: 40%). MS (ESI) m/z=849.8 [M+H]⁺.

### Example 68: 6-(2-(2-(2-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-2-oxoethoxy)acetamido)acetamido)-4-((2-methoxy-3-(1-methyl-1H-1,2,4-triazol-3-yl)phenyl)amino)-N-methylpyridazine-3-carboxamide (CPD-025)

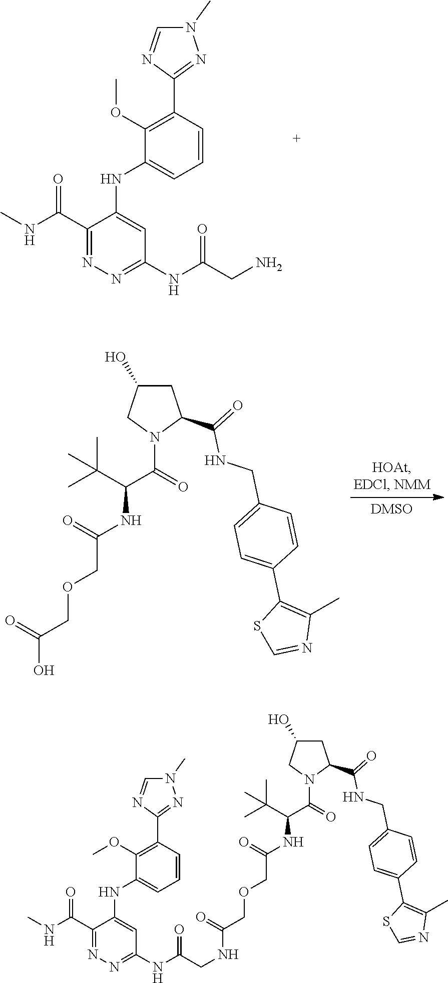

CPD-025 was synthesized following the same procedure for preparing CPD-042 (3.22 mg, yield: 17.6%). MS (ESI) m/z=940.7 [M+H]⁺.

### Example 69: 6-(2-(3-(3-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-3-oxopropoxy)propanamido)acetamido)-4-((2-methoxy-3-(1-methyl-1H-1,2,4-triazol-3-yl)phenyl)amino)-N-methylpyridazine-3-carboxamide (CPD-026)

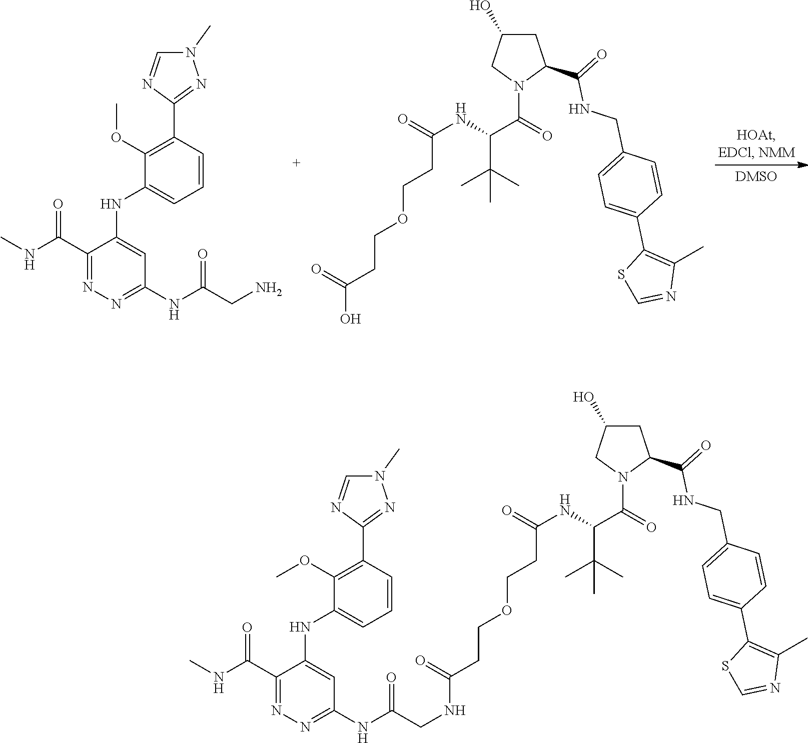

CPD-026 was synthesized following the same procedure for preparing CPD-042 (2.52 mg, yield: 13.2%). MS (ESI) m/z=968.8 [M+H]⁺.

### Example 70: 6-((S)-13-((2S,4R)-4-Hydroxy-2-((4-(4-methylthiazol-5-yl)benzyl)carbamoyl)pyrrolidine-1-carbonyl)-14,14-dimethyl-4,11-dioxo-6,9-dioxa-3,12-diazapentadecanamido)-4-((2-methoxy-3-(1-methyl-1H-1,2,4-triazol-3-yl)phenyl)amino)-N-methylpyridazine-3-carboxamide (CPD-027)

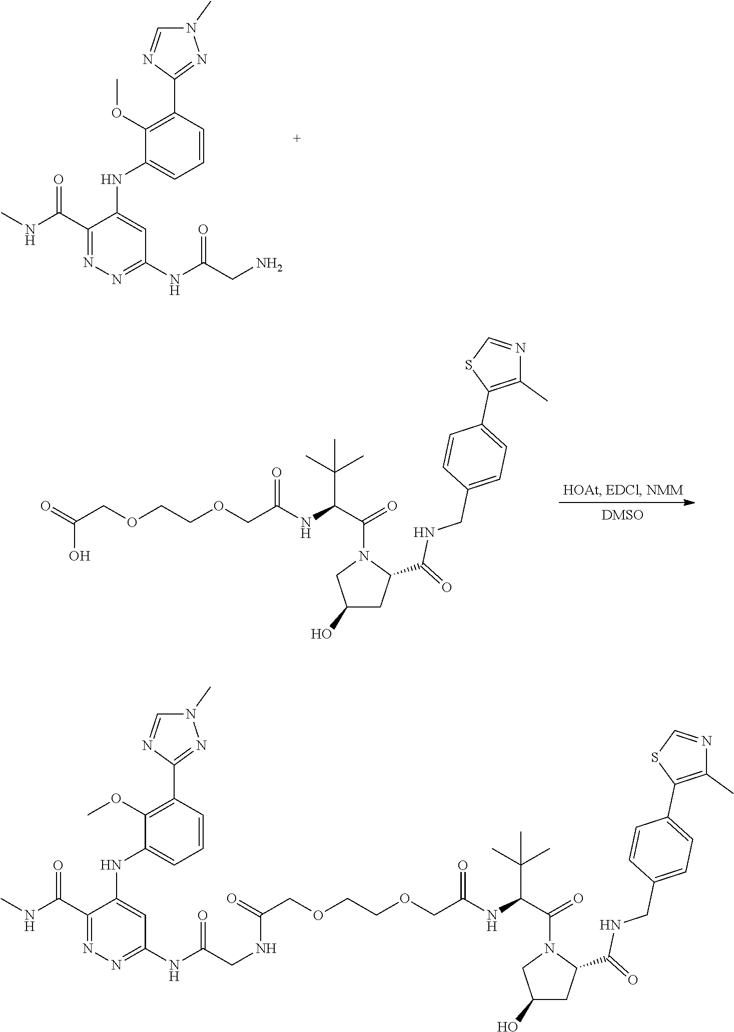

CPD-027 was synthesized following the same procedure for preparing CPD-042 (2.38 mg, yield: 11.9%). MS (ESI) m/z=984.8 [M+H]⁺.

### Example 71: 6-((S)-15-((2S,4R)-4-Hydroxy-2-((4-(4-methylthiazol-5-yl)benzyl)carbamoyl)pyrrolidine-1-carbonyl)-16,16-dimethyl-4,13-dioxo-7,10-dioxa-3,14-diazaheptadecanamido)-4-((2-methoxy-3-(1-methyl-1H-1,2,4-triazol-3-yl)phenyl)amino)-N-methylpyridazine-3-carboxamide (CPD-028)

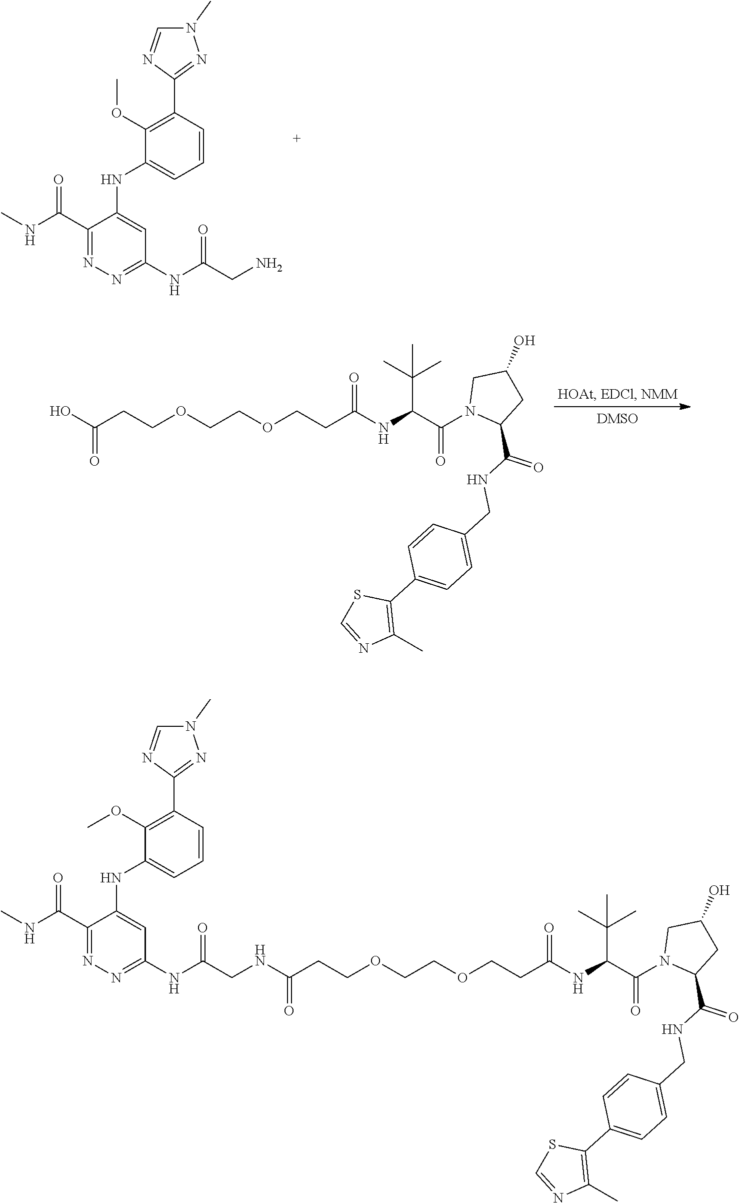 [SMILES: CNC(=O)c1nnc(NC(=O)CN)cc1Nc1cccc(-c2ncn(C)n2)c1OC ; "+" ; Cc1ncsc1-c1ccc(CNC(=O)[C@@H]2C[C@@H](O)CN2C(=O)[C@@H](NC(=O)CCOCCOCCC(=O)O)C(C)(C)C)cc1 ; CNC(=O)c1nnc(NC(=O)CNC(=O)CCOCCOCCC(=O)N[C@H](C(=O)N2C[C@H](O)C[C@H]2C(=O)NCc2ccc(-c3scnc3C)cc2)C(C)(C)C)cc1Nc1cccc(-c2ncn(C)n2)c1OC]

CPD-028 was synthesized following the same procedure for preparing CPD-042 (4.52 mg, yield: 20.9%). MS (ESI) m/z=1012.8 [M+H]

### Example 72: 6-((S)-16-((2S,4R)-4-Hydroxy-2-((4-(4-methylthiazol-5-yl)benzyl)carbamoyl)pyrrolidine-1-carbonyl)-17,17-dimethyl-4,14-dioxo-6,9,12-trioxa-3,15-diazaoctadecanamido)-4-((2-methoxy-3-(1-methyl-1H-1,2,4-triazol-3-yl)phenyl)amino)-N-methylpyridazine-3-carboxamide (CPD-029)

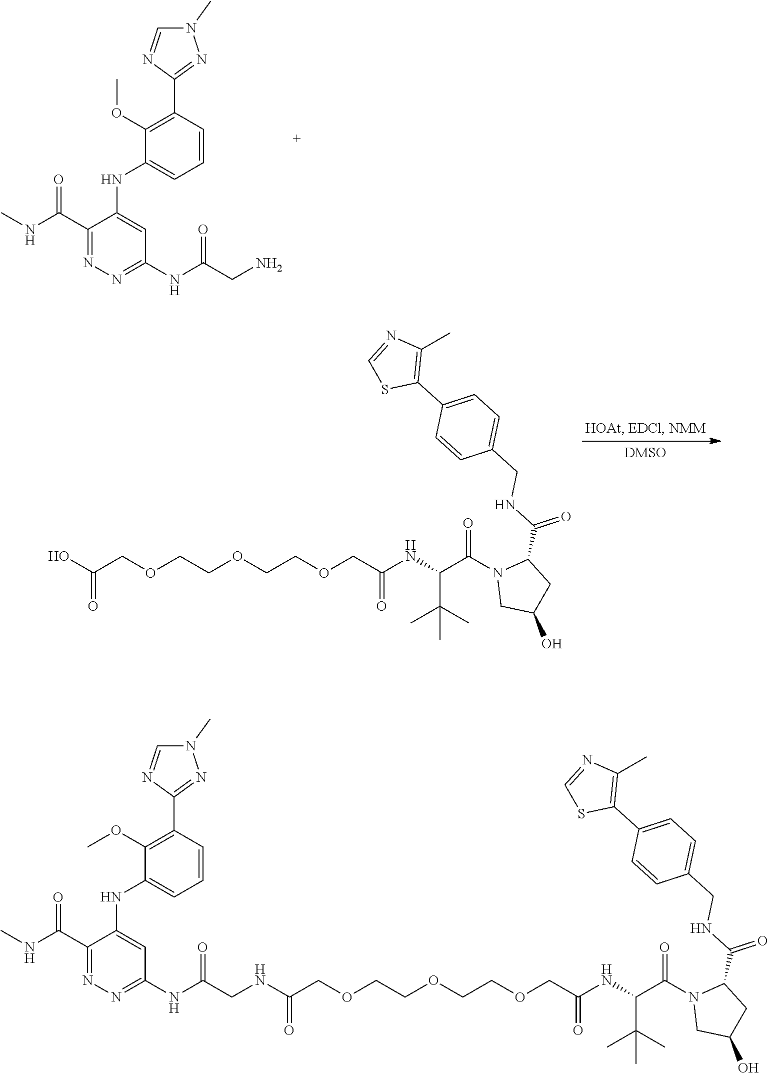 [SMILES: CNC(=O)c1nnc(NC(=O)CN)cc1Nc1cccc(-c2ncn(C)n2)c1OC ; "+" ; Cc1ncsc1-c1ccc(CNC(=O)[C@@H]2C[C@@H](O)CN2C(=O)[C@@H](NC(=O)COCCOCCOCC(=O)O)C(C)(C)C)cc1 ; CNC(=O)c1nnc(NC(=O)CNC(=O)COCCOCCOCC(=O)N[C@H](C(=O)N2C[C@H](O)C[C@H]2C(=O)NCc2ccc(-c3scnc3C)cc2)C(C)(C)C)cc1Nc1cccc(-c2ncn(C)n2)c1OC]

CPD-029 was synthesized following the same procedure for preparing CPD-042 (1.96 mg, yield: 10.4%). MS (ESI) m/z=1028.7 [M+H]⁺.

### Example 73: 6-((S)-18-((2S,4R)-4-Hydroxy-2-((4-(4-methylthiazol-5-yl)benzyl)carbamoyl)pyrrolidine-1-carbonyl)-19,19-dimethyl-4,16-dioxo-7,10,13-trioxa-3,17-diazaicosanamido)-4-((2-methoxy-3-(1-methyl-1H-1,2,4-triazol-3-yl)phenyl)amino)-N-methylpyridazine-3-carboxamide (CPD-030)

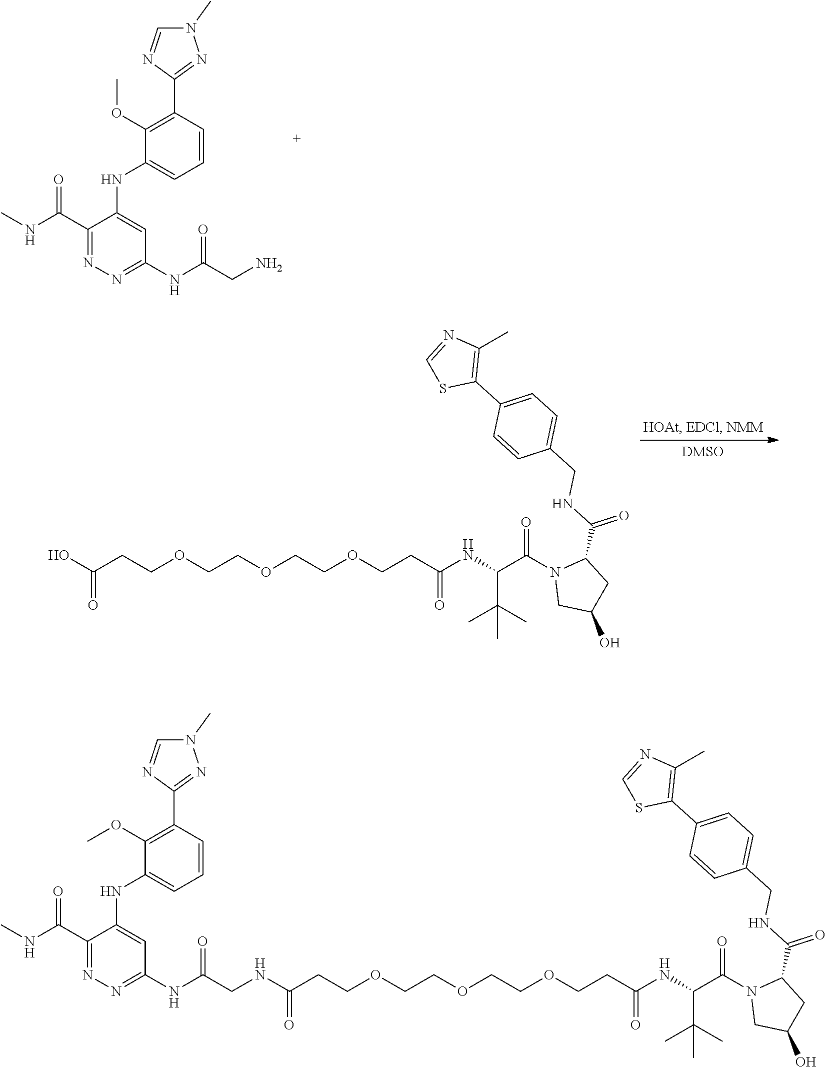 [SMILES: CNC(=O)c1nnc(NC(=O)CN)cc1Nc1cccc(-c2ncn(C)n2)c1OC ; "+" ; Cc1ncsc1-c1ccc(CNC(=O)[C@@H]2C[C@@H](O)CN2C(=O)[C@@H](NC(=O)CCOCCOCCOCCC(=O)O)C(C)(C)C)cc1 ; CNC(=O)c1nnc(NC(=O)CNC(=O)CCOCCOCCOCCC(=O)N[C@H](C(=O)N2C[C@H](O)C[C@H]2C(=O)NCc2ccc(-c3scnc3C)cc2)C(C)(C)C)cc1Nc1cccc(-c2ncn(C)n2)c1OC]

CPD-030 was synthesized following the same procedure for preparing CPD-042 (2.28 mg, yield: 11.6%). MS (ESI) m/z=1056.9 [M+H]⁺.

### Example 74: N¹—((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)-N¹⁶-(2-((5-((2-methoxy-3-(1-methyl-1H-1,2,4-triazol-3-yl)phenyl)amino)-6-(methylcarbamoyl)pyridazin-3-yl)amino)-2-oxoethyl)-4,7,10,13-tetraoxahexadecanediamide (CPD-031)

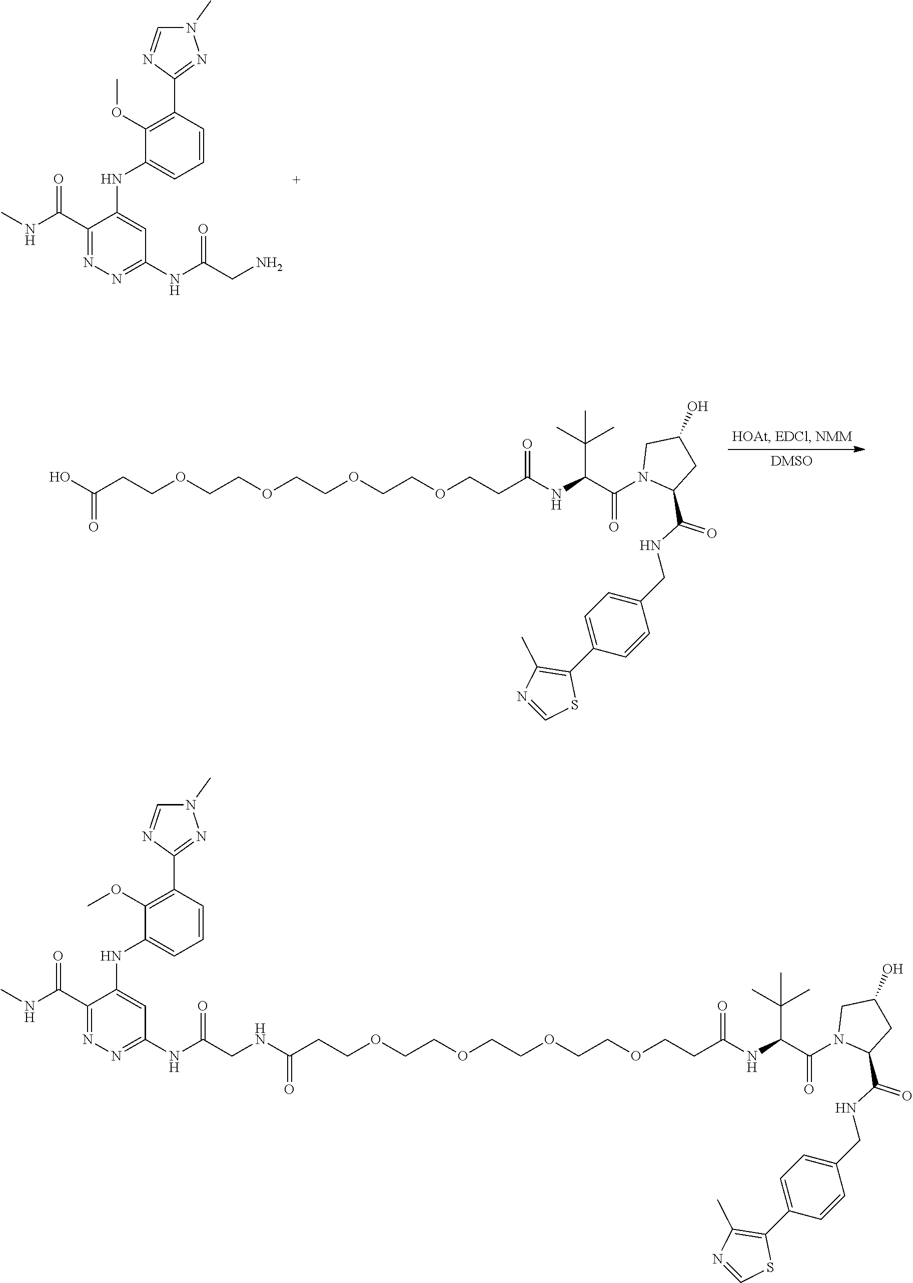 [SMILES: CNC(=O)c1nnc(NC(=O)CN)cc1Nc1cccc(-c2ncn(C)n2)c1OC ; "+" ; Cc1ncsc1-c1ccc(CNC(=O)[C@@H]2C[C@@H](O)CN2C(=O)[C@@H](NC(=O)CCOCCOCCOCCOCCC(=O)O)C(C)(C)C)cc1 ; CNC(=O)c1nnc(NC(=O)CNC(=O)CCOCCOCCOCCOCCC(=O)N[C@H](C(=O)N2C[C@H](O)C[C@H]2C(=O)NCc2ccc(-c3scnc3C)cc2)C(C)(C)C)cc1Nc1cccc(-c2ncn(C)n2)c1OC]

CPD-031 was synthesized following the same procedure for preparing CPD-042 (2.63 mg, yield: 12.8%). MS (ESI) m/z=1100.9 [M+H]⁺.

### Example 75: N¹—((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)-N¹⁷-(2-((5-((2-methoxy-3-(1-methyl-1H-1,2,4-triazol-3-yl)phenyl)amino)-6-(methylcarbamoyl)pyridazin-3-yl)amino)-2-oxoethyl)-3,6,9,12,15-pentaoxaheptadecanediamide (CPD-032)

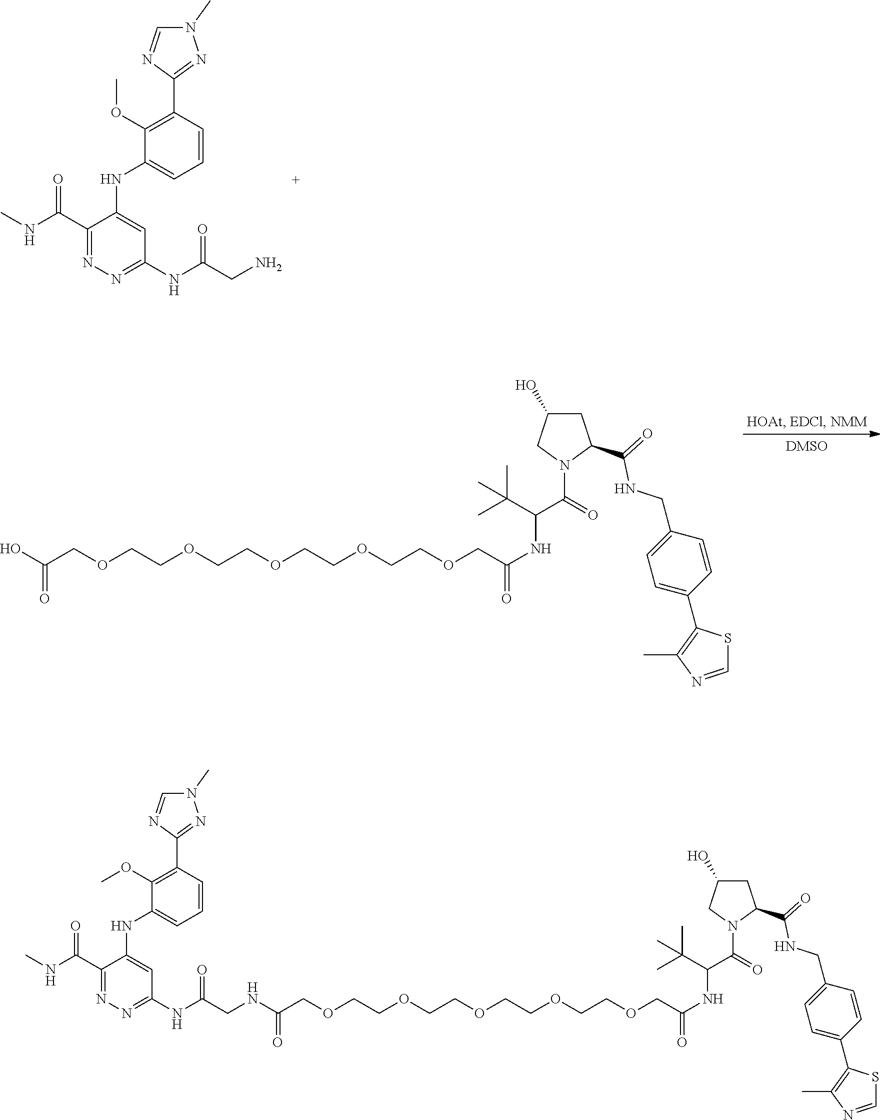 [SMILES: CNC(=O)c1nnc(NC(=O)CN)cc1Nc1cccc(-c2ncn(C)n2)c1OC ; "+" ; Cc1ncsc1-c1ccc(CNC(=O)[C@@H]2C[C@@H](O)CN2C(=O)C(NC(=O)COCCOCCOCCOCCOCC(=O)O)C(C)(C)C)cc1 ; CNC(=O)c1nnc(NC(=O)CNC(=O)COCCOCCOCCOCCOCC(=O)NC(C(=O)N2C[C@H](O)C[C@H]2C(=O)NCc2ccc(-c3scnc3C)cc2)C(C)(C)C)cc1Nc1cccc(-c2ncn(C)n2)c1OC]

CPD-032 was synthesized following the same procedure for preparing CPD-042 (2.25 mg, yield: 10.5%). MS (ESI) m/z=1116.8 [M+H]⁺.

### Example 76: N¹—((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)-N¹⁹-(2-((5-((2-methoxy-3-(1-methyl-1H-1,2,4-triazol-3-yl)phenyl)amino)-6-(methylcarbamoyl)pyridazin-3-yl)amino)-2-oxoethyl)-4,7,10,13,16-pentaoxanonadecanediamide (CPD-033)

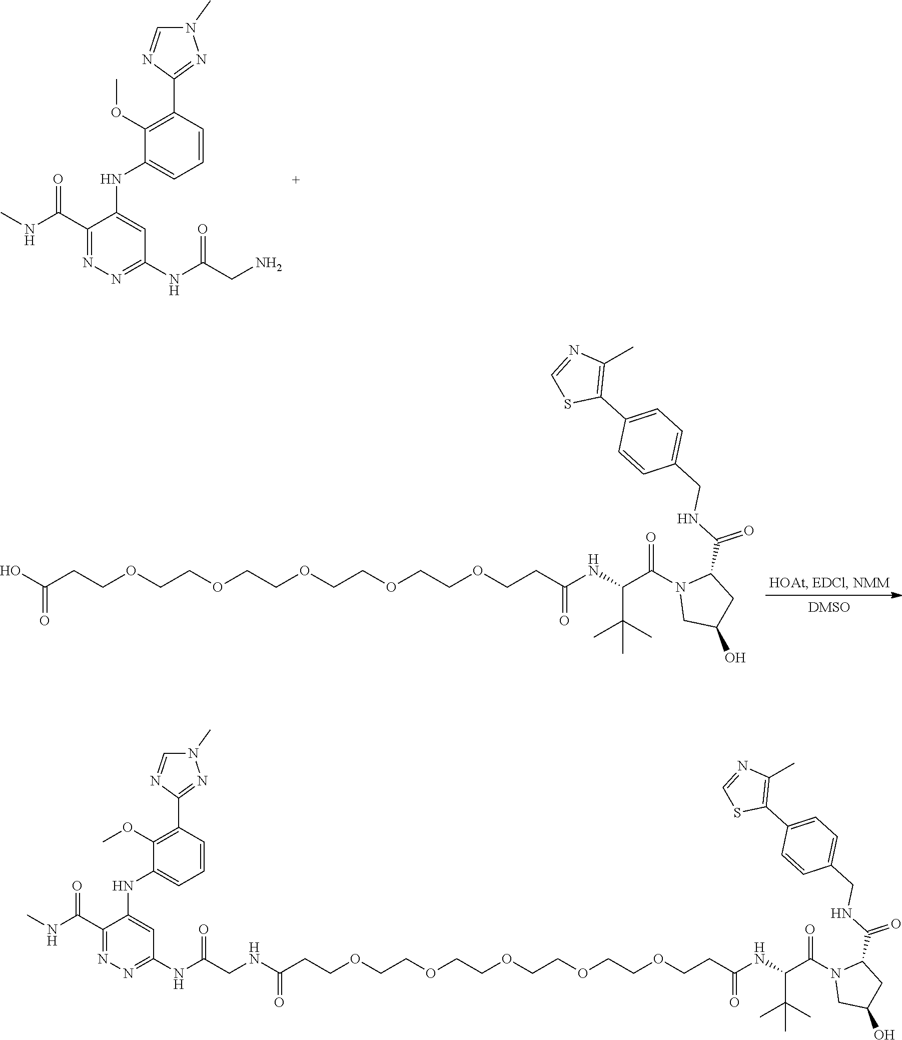 [SMILES: CNC(=O)c1nnc(NC(=O)CN)cc1Nc1cccc(-c2ncn(C)n2)c1OC ; "+" ; Cc1ncsc1-c1ccc(CNC(=O)[C@@H]2C[C@@H](O)CN2C(=O)[C@@H](NC(=O)CCOCCOCCOCCOCCOCCC(=O)O)C(C)(C)C)cc1 ; CNC(=O)c1nnc(NC(=O)CNC(=O)CCOCCOCCOCCOCCOCCC(=O)N[C@H](C(=O)N2C[C@H](O)C[C@H]2C(=O)NCc2ccc(-c3scnc3C)cc2)C(C)(C)C)cc1Nc1cccc(-c2ncn(C)n2)c1OC]

CPD-033 was synthesized following the same procedure for preparing CPD-042 (2.63 mg, yield: 11.8%₀). MS (ESI) m/z=1144.9 [M+H]Y.

### Example 77: N¹—((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)-N⁴-(2-((5-((2-methoxy-3-(1-methyl-1H-1,2,4-triazol-3-yl)phenyl)amino)-6-(methylcarbamoyl)pyridazin-3-yl)amino)-2-oxoethyl)succinamide (CPD-034)

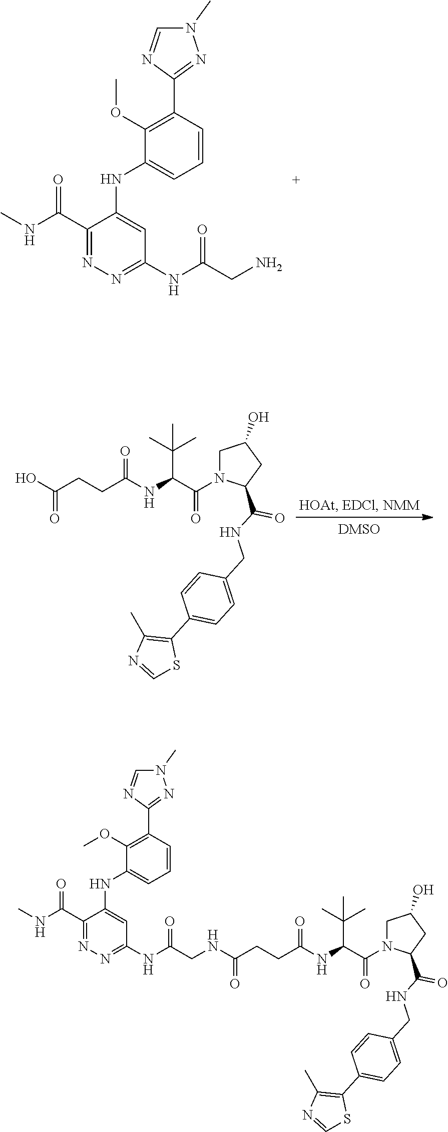 [SMILES: CNC(=O)c1nnc(NC(=O)CN)cc1Nc1cccc(-c2ncn(C)n2)c1OC ; "+" ; Cc1ncsc1-c1ccc(CNC(=O)[C@@H]2C[C@@H](O)CN2C(=O)[C@@H](NC(=O)CCC(=O)O)C(C)(C)C)cc1 ; CNC(=O)c1nnc(NC(=O)CNC(=O)CCC(=O)N[C@H](C(=O)N2C[C@H](O)C[C@H]2C(=O)NCc2ccc(-c3scnc3C)cc2)C(C)(C)C)cc1Nc1cccc(-c2ncn(C)n2)c1OC]

CPD-034 was synthesized following the same procedure for preparing CPD-042 (1.10 mg, yield: 6.1%). MS (ESI) m/z=924.7 [M+H]⁺.

### Example 78: N¹—((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)-N⁵-(2-((5-((2-methoxy-3-(1-methyl-1H-1,2,4-triazol-3-yl)phenyl)amino)-6-(methylcarbamoyl)pyridazin-3-yl)amino)-2-oxoethyl)glutaramide (CPD-035)

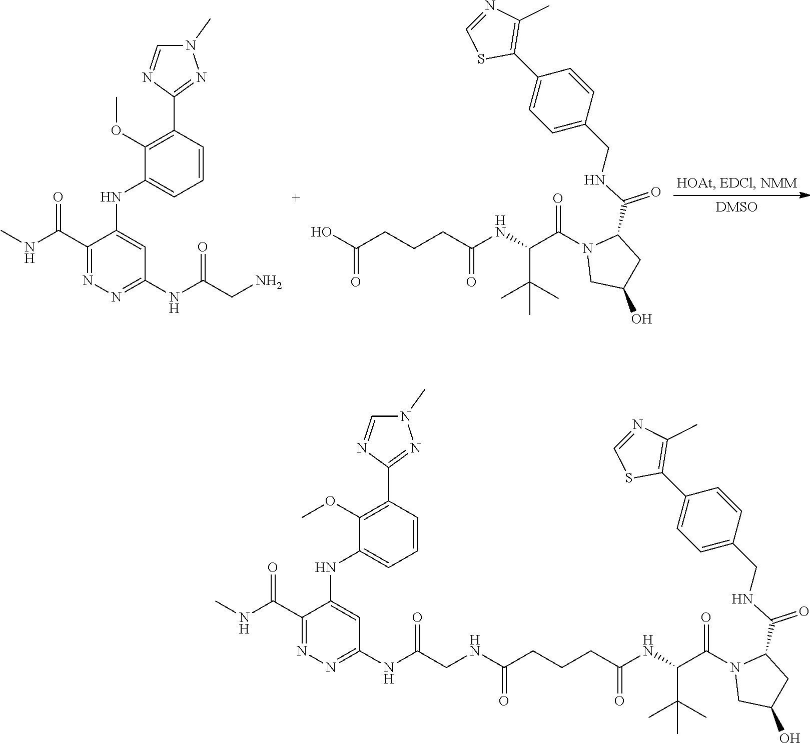

CPD-035 was synthesized following the same procedure for preparing CPD-042 (1.14 g, yield: 6.3%). MS (ESI) m/z=938.7 [M+H]⁺.

### Example 79: N¹-((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)-N⁶-(2-((5-((2-methoxy-3-(1-methyl-1H-1,2,4-triazol-3-yl)phenyl)amino)-6-(methylcarbamoyl)pyridazin-3-yl)amino)-2-oxoethyl)adipamide (CPD-036)

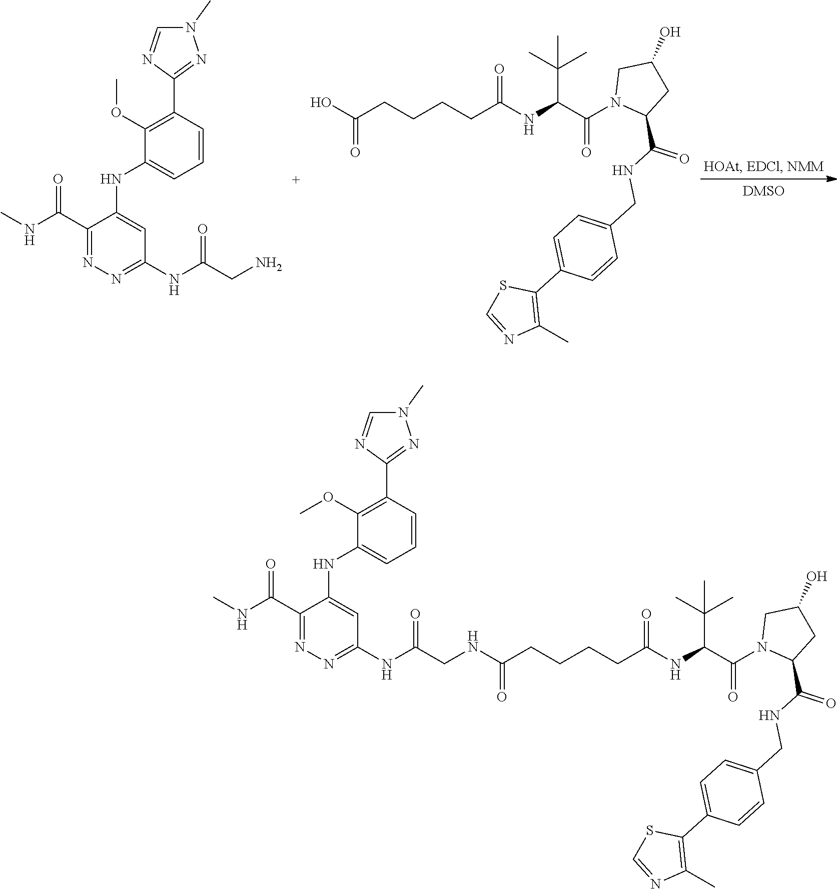 [SMILES: CNC(=O)c1nnc(NC(=O)CN)cc1Nc1cccc(-c2ncn(C)n2)c1OC ; "+" ; Cc1ncsc1-c1ccc(CNC(=O)[C@@H]2C[C@@H](O)CN2C(=O)[C@@H](NC(=O)CCCCC(=O)O)C(C)(C)C)cc1 ; CNC(=O)c1nnc(NC(=O)CNC(=O)CCCCC(=O)N[C@H](C(=O)N2C[C@H](O)C[C@H]2C(=O)NCc2ccc(-c3scnc3C)cc2)C(C)(C)C)cc1Nc1cccc(-c2ncn(C)n2)c1OC]

CPD-036 was synthesized following the same procedure for preparing CPD-042 (1.46 mg, yield: 7.9%). MS (ESI) m/z=952.7 [M+H]⁺.

### Example 80: N¹—((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)-N⁷-(2-((5-((2-methoxy-3-(1-methyl-1H-1,2,4-triazol-3-yl)phenyl)amino)-6-(methylcarbamoyl)pyridazin-3-yl)amino)-2-oxoethyl)heptanediamide (CPD-037)

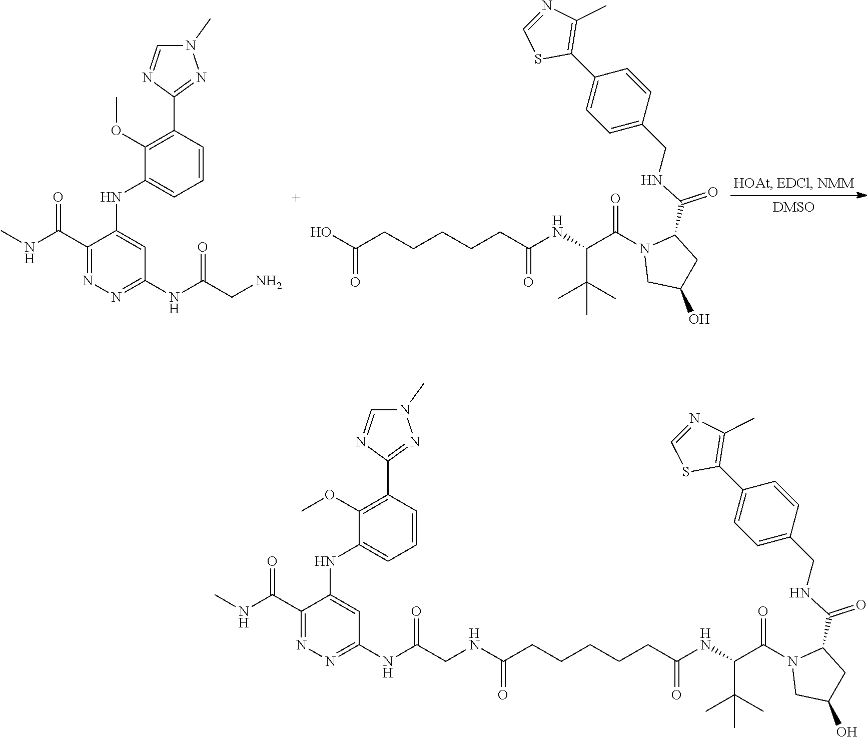

CPD-037 was synthesized following the same procedure for preparing CPD-042 (1.56 mg, yield: 8.3%). MS (ESI) m/z=966.8 [M+H]⁺.

### Example 81: N¹-((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)-N$-(2-((5-((2-methoxy-3-(1-methyl-1H-1,2,4-triazol-3-yl)phenyl)amino)-6-(methylcarbamoyl)pyridazin-3-yl)amino)-2-oxoethyl)octanediamide (CPD-038)

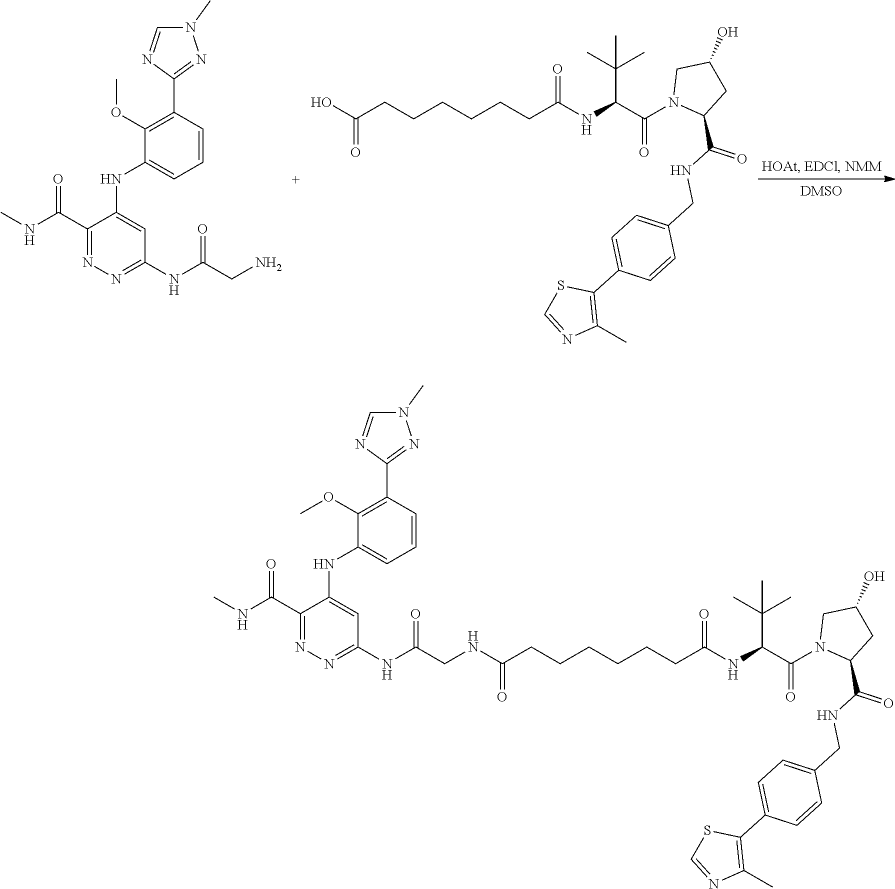 [SMILES: CNC(=O)c1nnc(NC(=O)CN)cc1Nc1cccc(-c2ncn(C)n2)c1OC ; "+" ; Cc1ncsc1-c1ccc(CNC(=O)[C@@H]2C[C@@H](O)CN2C(=O)[C@@H](NC(=O)CCCCCCC(=O)O)C(C)(C)C)cc1 ; CNC(=O)c1nnc(NC(=O)CNC(=O)CCCCCCC(=O)N[C@H](C(=O)N2C[C@H](O)C[C@H]2C(=O)NCc2ccc(-c3scnc3C)cc2)C(C)(C)C)cc1Nc1cccc(-c2ncn(C)n2)c1OC]

CPD-038 was synthesized following the same procedure for preparing CPD-042 (2.10 mg, yield: 11.1%). MS (ESI) m/z=980.8 [M+H]⁺.

### Example 82: N¹-((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)-N⁹-(2-((5-((2-methoxy-3-(1-methyl-1H-1,2,4-triazol-3-yl)phenyl)amino)-6-(methylcarbamoyl)pyridazin-3-yl)amino)-2-oxoethyl)nonanediamide (CPD-039)

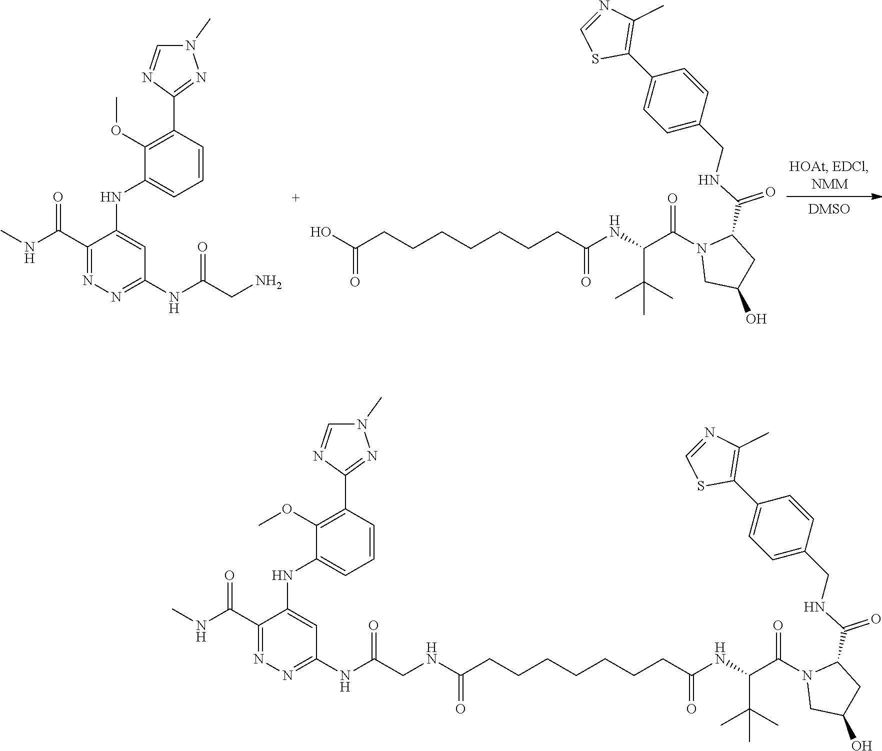

CPD-039 was synthesized following the same procedure for preparing CPD-042 (3.49 mg, yield: 18.1%). MS (ESI) m/z=994.8 [M+H]⁺.

### Example 83: N¹—((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)-N¹⁰-(2-((5-((2-methoxy-3-(1-methyl-1H-1,2,4-triazol-3-yl)phenyl)amino)-6-(methylcarbamoyl)pyridazin-3-yl)amino)-2-oxoethyl)decanediamide (CPD-040)

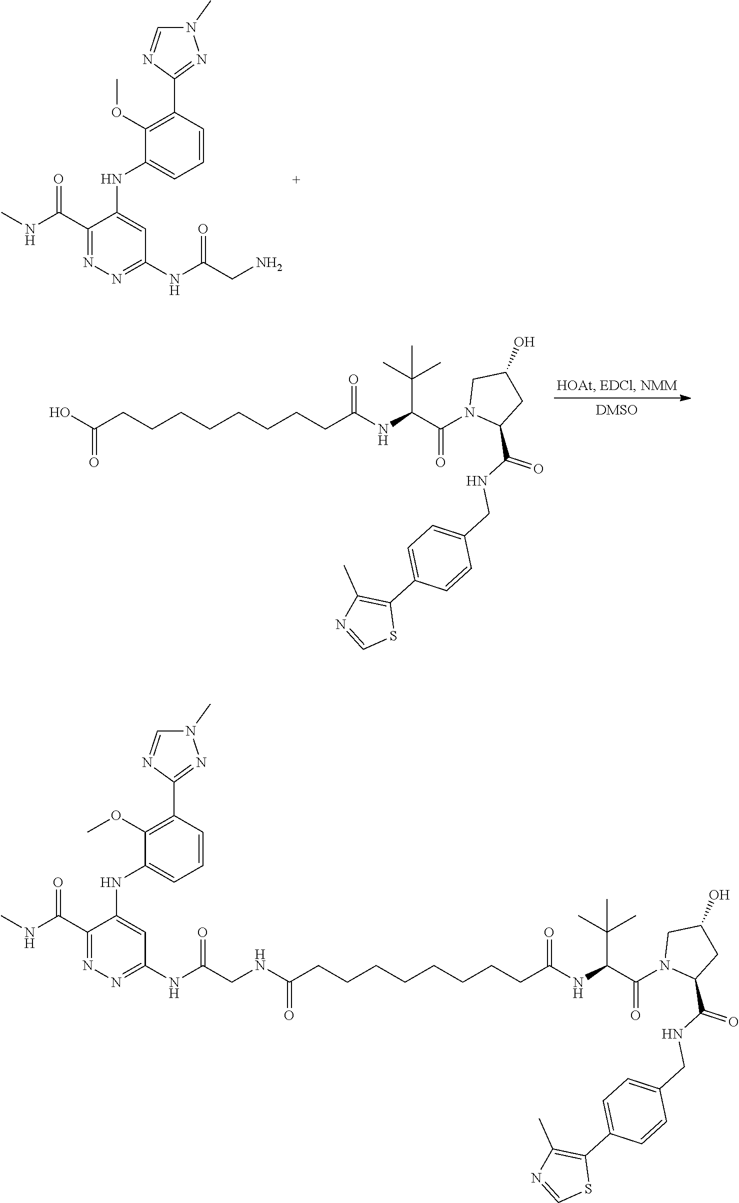 [SMILES: CNC(=O)c1nnc(NC(=O)CN)cc1Nc1cccc(-c2ncn(C)n2)c1OC ; "+" ; Cc1ncsc1-c1ccc(CNC(=O)[C@@H]2C[C@@H](O)CN2C(=O)[C@@H](NC(=O)CCCCCCCCC(=O)O)C(C)(C)C)cc1 ; CNC(=O)c1nnc(NC(=O)CNC(=O)CCCCCCCCC(=O)N[C@H](C(=O)N2C[C@H](O)C[C@H]2C(=O)NCc2ccc(-c3scnc3C)cc2)C(C)(C)C)cc1Nc1cccc(-c2ncn(C)n2)c1OC]

CPD-040 was synthesized following the same procedure for preparing CPD-042 (3.97 mg, yield: 20.3%). MS (ESI) m/z=1008.7 [M+H]⁺.

### Example 84: N¹—((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)-N¹¹-(2-((5-((2-methoxy-3-(1-methyl-1H-1,2,4-triazol-3-yl)phenyl)amino)-6-(methylcarbamoyl)pyridazin-3-yl)amino)-2-oxoethyl)undecanediamide (CPD-041)

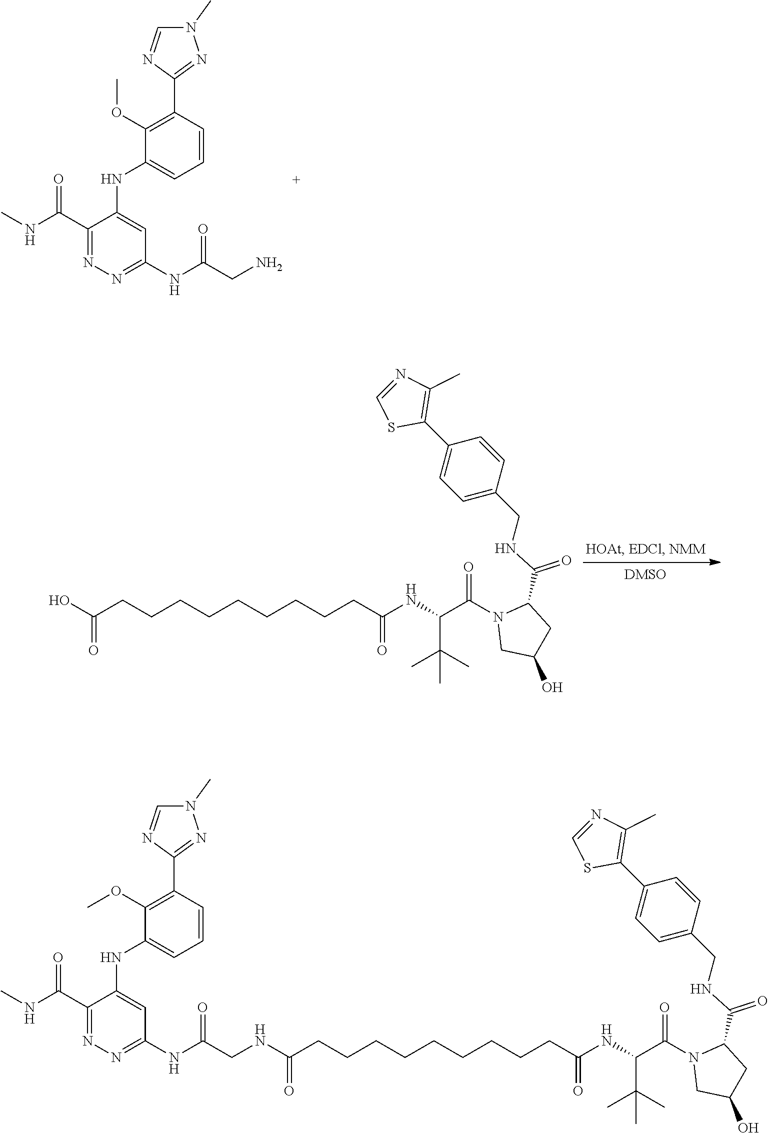 [SMILES: CNC(=O)c1nnc(NC(=O)CN)cc1Nc1cccc(-c2ncn(C)n2)c1OC ; "+" ; Cc1ncsc1-c1ccc(CNC(=O)[C@@H]2C[C@@H](O)CN2C(=O)[C@@H](NC(=O)CCCCCCCCCC(=O)O)C(C)(C)C)cc1 ; CNC(=O)c1nnc(NC(=O)CNC(=O)CCCCCCCCCC(=O)N[C@H](C(=O)N2C[C@H](O)C[C@H]2C(=O)NCc2ccc(-c3scnc3C)cc2)C(C)(C)C)cc1Nc1cccc(-c2ncn(C)n2)c1OC]

CPD-041 was synthesized following the same procedure for preparing CPD-042 (2.02 mg, yield: 10.2%). MS (ESI) m/z=1022.8 [M+H]⁺.

### Example 85: 6-(2-(2-((2-(2,6-Dioxopiperidin-3-yl)-1,3-dioxoisoindolin-4-yl)amino)acetamido)acetamido)-4-((2-methoxy-3-(1-methyl-1H-1,2,4-triazol-3-yl)phenyl)amino)-N-methylpyridazine-3-carboxamide (CPD-042)

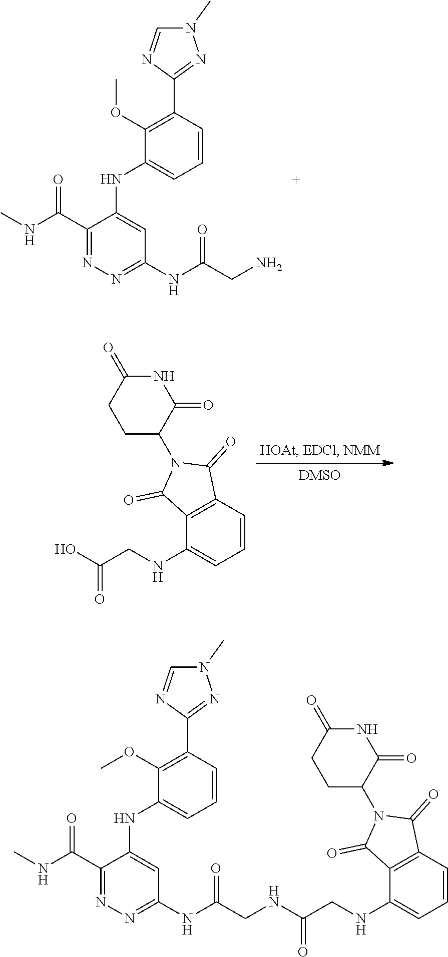 [SMILES: CNC(=O)c1nnc(NC(=O)CN)cc1Nc1cccc(-c2ncn(C)n2)c1OC ; "+" ; O=C(O)CNc1cccc2c1C(=O)N(C1CCC(=O)NC1=O)C2=O ; CNC(=O)c1nnc(NC(=O)CNC(=O)CNc2cccc3c2C(=O)N(C2CCC(=O)NC2=O)C3=O)cc1Nc1cccc(-c2ncn(C)n2)c1OC]

To a mixture of 6-(2-aminoacetamido)-4-((2-methoxy-3-(1-methyl-1H-1,2,4-triazol-3-yl)phenyl)amino)-N-methylpyridazine-3-carboxamide (8 mg, 0.019 mmol), HOAt (5 mg, 0.038 mmol) and EDCI (7 mg, 0.038 mmol) in DMSO (1 mL) were added N-methylmorpholine (10 mg, 0.095 mmol) and (2-(2,6-dioxopiperidin-3-yl)-1,3-dioxoisoindolin-4-yl)glycine (6 mg, 0.019 mmol). After the mixture was stirred at 25° C. for 16 h, it was purified by reverse-phase chromatography to give the title compound (1.18 mg, yield: 8.4%) as a yellow solid. MS (ESI) m/z=725.5 [M+H]⁺.

### Example 86: 6-(2-(3-((2-(2,6-Dioxopiperidin-3-yl)-1,3-dioxoisoindolin-4-yl)amino)propanamido)acetamido)-4-((2-methoxy-3-(1-methyl-1H-1,2,4-triazol-3-yl)phenyl)amino)-N-methylpyridazine-3-carboxamide (CPD-043)

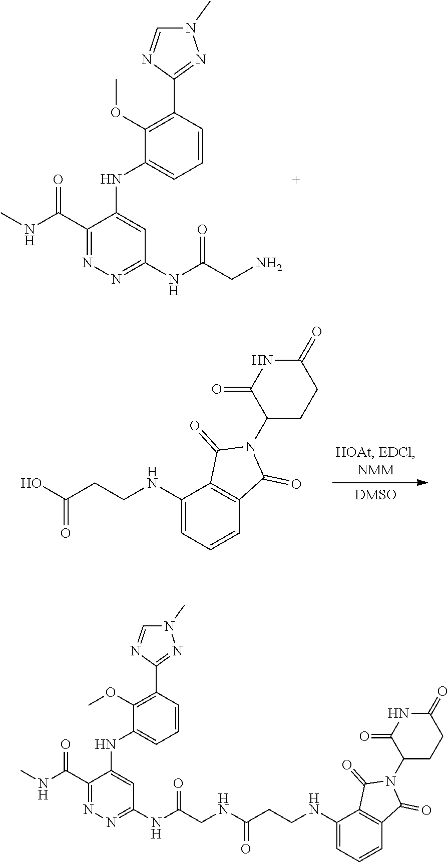 [SMILES: CNC(=O)c1nnc(NC(=O)CN)cc1Nc1cccc(-c2ncn(C)n2)c1OC ; "+" ; O=C(O)CCNc1cccc2c1C(=O)N(C1CCC(=O)NC1=O)C2=O ; CNC(=O)c1nnc(NC(=O)CNC(=O)CCNc2cccc3c2C(=O)N(C2CCC(=O)NC2=O)C3=O)cc1Nc1cccc(-c2ncn(C)n2)c1OC]

CPD-043 was synthesized following the same procedure for preparing CPD-042 (1.22 mg, yield: 8.5%). MS (ESI) m/z=739.6 [M+H]⁺.

### Example 87: 6-(2-(4-((2-(2,6-Dioxopiperidin-3-yl)-1,3-dioxoisoindolin-4-yl)amino)butanamido)acetamido)-4-((2-methoxy-3-(1-methyl-1H-1,2,4-triazol-3-yl)phenyl)amino)-N-methylpyridazine-3-carboxamide (CPD-044)

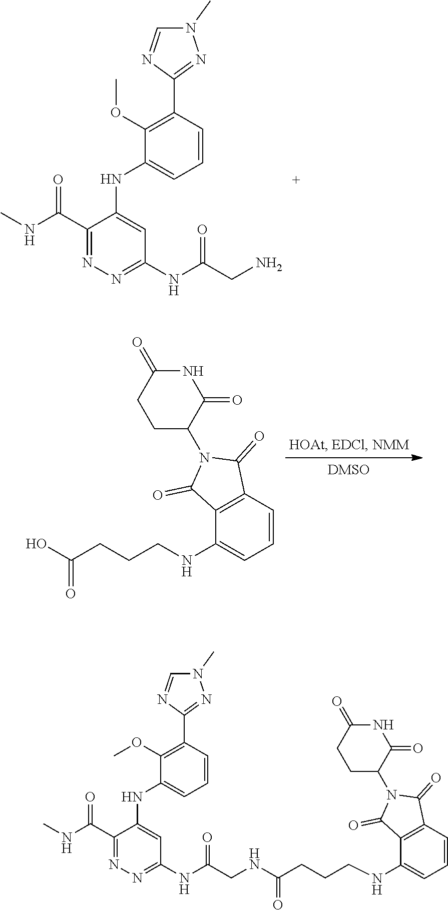 [SMILES: CNC(=O)c1nnc(NC(=O)CN)cc1Nc1cccc(-c2ncn(C)n2)c1OC ; "+" ; O=C(O)CCCNc1cccc2c1C(=O)N(C1CCC(=O)NC1=O)C2=O ; CNC(=O)c1nnc(NC(=O)CNC(=O)CCCNc2cccc3c2C(=O)N(C2CCC(=O)NC2=O)C3=O)cc1Nc1cccc(-c2ncn(C)n2)c1OC]

CPD-044 was synthesized following the same procedure for preparing CPD-042 (2.09 mg, yield: 11.7%). MS (ESI) m/z=753.5 [M+H]⁺.

### Example 88: 6-(2-(5-((2-(2,6-Dioxopiperidin-3-yl)-1,3-dioxoisoindolin-4-yl)amino)pentanamido)acetamido)-4-((2-methoxy-3-(1-methyl-1H-1,2,4-triazol-3-yl)phenyl)amino)-N-methylpyridazine-3-carboxamide (CPD-045)

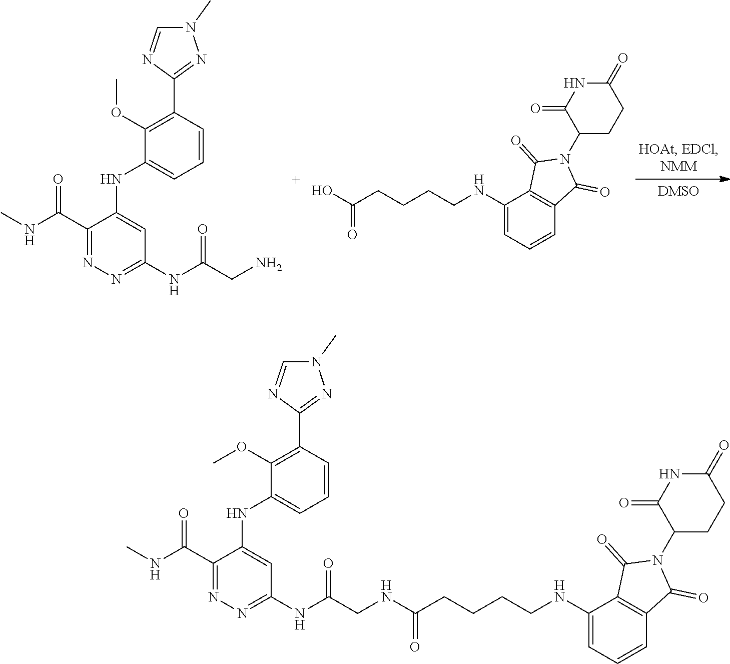

CPD-045 was synthesized following the same procedure for preparing CPD-042 (2.47 mg, yield: 16.6%). MS (ESI) m/z=767.6 [M+H]⁺.

### Example 89: 6-(2-(6-((2-(2,6-Dioxopiperidin-3-yl)-1,3-dioxoisoindolin-4-yl)amino)hexanamido)acetamido)-4-((2-methoxy-3-(1-methyl-1H-1,2,4-triazol-3-yl)phenyl)amino)-N-methylpyridazine-3-carboxamide (CPD-046)

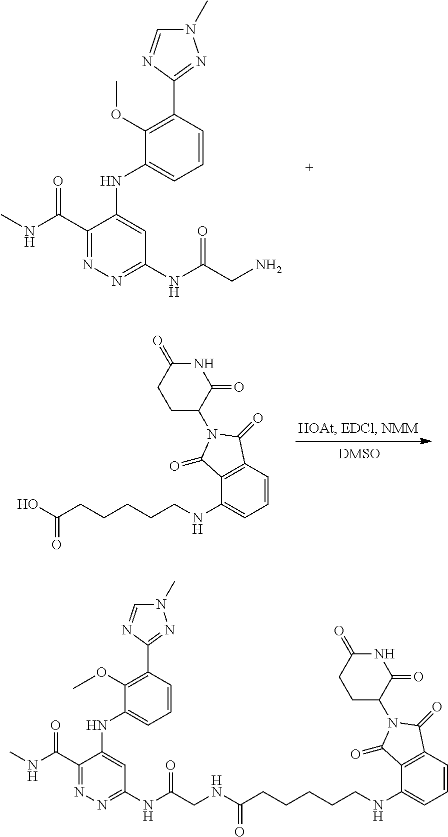 [SMILES: CNC(=O)c1nnc(NC(=O)CN)cc1Nc1cccc(-c2ncn(C)n2)c1OC ; O=C(O)CCCCCNc1cccc2c1C(=O)N(C1CCC(=O)NC1=O)C2=O ; CNC(=O)c1nnc(NC(=O)CNC(=O)CCCCCNc2cccc3c2C(=O)N(C2CCC(=O)NC2=O)C3=O)cc1Nc1cccc(-c2ncn(C)n2)c1OC]

CPD-046 was synthesized following the same procedure for preparing CPD-042 (1.73 mg, yield: 11.4%). MS (ESI) m/z=781.6 [M+H]⁺.

### Example 90: 6-(2-(7-((2-(2,6-Dioxopiperidin-3-yl)-1,3-dioxoisoindolin-4-yl)amino)heptanamido)acetamido)-4-((2-methoxy-3-(1-methyl-1H-1,2,4-triazol-3-yl)phenyl)amino)-N-methylpyridazine-3-carboxamide (CPD-047)

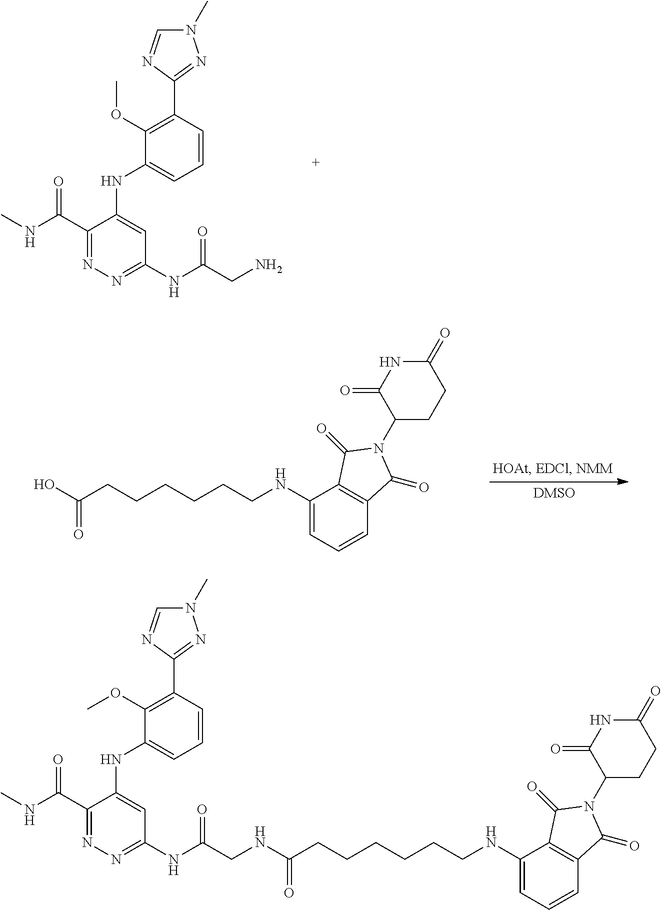 [SMILES: CNC(=O)c1nnc(NC(=O)CN)cc1Nc1cccc(-c2ncn(C)n2)c1OC ; O=C(O)CCCCCCNc1cccc2c1C(=O)N(C1CCC(=O)NC1=O)C2=O ; CNC(=O)c1nnc(NC(=O)CNC(=O)CCCCCCNc2cccc3c2C(=O)N(C2CCC(=O)NC2=O)C3=O)cc1Nc1cccc(-c2ncn(C)n2)c1OC]

CPD-047 was synthesized following the same procedure for preparing CPD-042 (2.00 mg, yield: 12.9%). MS (ESI) m/z=795.6 [M+H]⁺.

### Example 91: 6-(2-(8-((2-(2,6-Dioxopiperidin-3-yl)-1,3-dioxoisoindolin-4-yl)amino)octanamido)acetamido)-4-((2-methoxy-3-(1-methyl-1H-1,2,4-triazol-3-yl)phenyl)amino)-N-methylpyridazine-3-carboxamide (CPD-048)

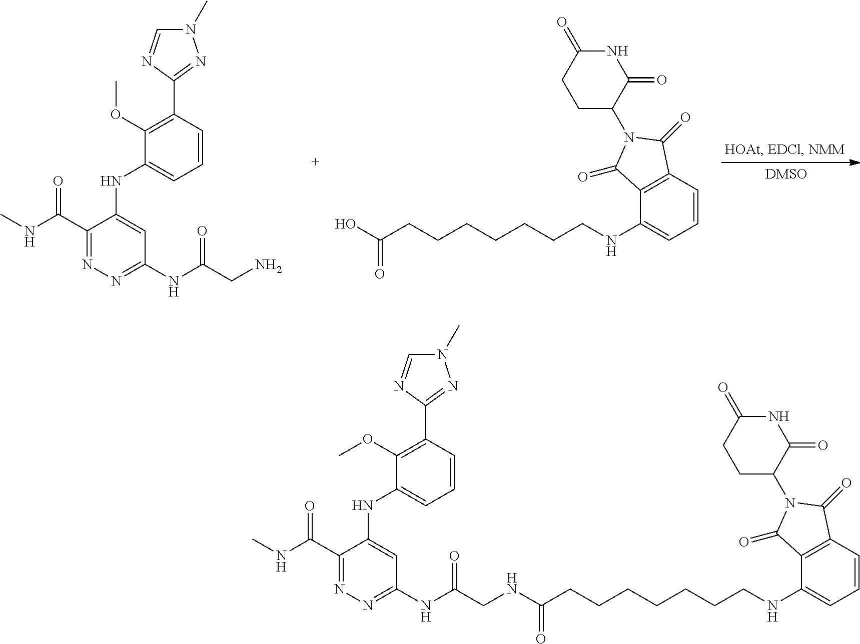 [SMILES: CNC(=O)c1nnc(NC(=O)CN)cc1Nc1cccc(-c2ncn(C)n2)c1OC ; O=C(O)CCCCCCCNc1cccc2c1C(=O)N(C1CCC(=O)NC1=O)C2=O ; CNC(=O)c1nnc(NC(=O)CNC(=O)CCCCCCCNc2cccc3c2C(=O)N(C2CCC(=O)NC2=O)C3=O)cc1Nc1cccc(-c2ncn(C)n2)c1OC]

CPD-048 was synthesized following the same procedure for preparing CPD-042 (3.13 mg, yield: 19.9%). MS (ESI) m/z=809.7 [M+H]⁺.

### Example 92: 6-(2-(3-(2-((2-(2,6-Dioxopiperidin-3-yl)-1,3-dioxoisoindolin-4-yl)amino)ethoxy)propanamido)acetamido)-4-((2-methoxy-3-(1-methyl-1H-1,2,4-triazol-3-yl)phenyl)amino)-N-methylpyridazine-3-carboxamide (CPD-049)

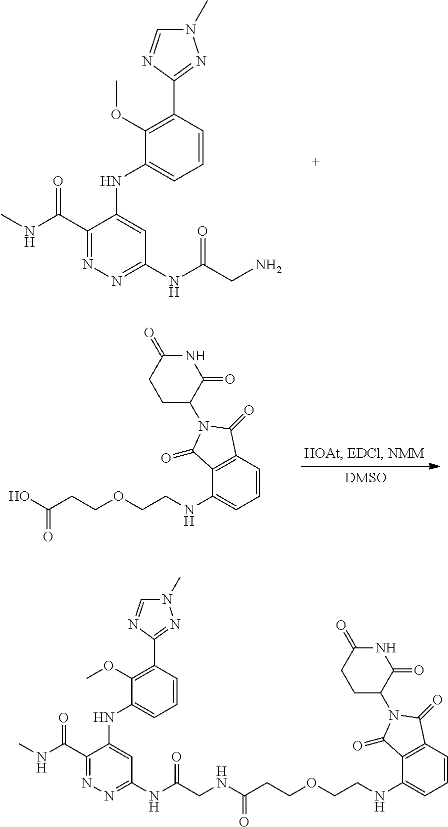 [SMILES: CNC(=O)c1nnc(NC(=O)CN)cc1Nc1cccc(-c2ncn(C)n2)c1OC ; O=C(O)CCOCCNc1cccc2c1C(=O)N(C1CCC(=O)NC1=O)C2=O ; CNC(=O)c1nnc(NC(=O)CNC(=O)CCOCCNc2cccc3c2C(=O)N(C2CCC(=O)NC2=O)C3=O)cc1Nc1cccc(-c2ncn(C)n2)c1OC]

CPD-049 was synthesized following the same procedure for preparing CPD-042 (2.78 mg, yield: 18.3%). MS (ESI) m/z=783.6 [M+H]⁺.

### Example 93: 6-(2-(3-(2-(2-((2-(2,6-Dioxopiperidin-3-yl)-1,3-dioxoisoindolin-4-yl)amino)ethoxy)ethoxy)propanamido)acetamido)-4-((2-methoxy-3-(1-methyl-1H-1,2,4-triazol-3-yl)phenyl)amino)-N-methylpyridazine-3-carboxamide (CPD-050)

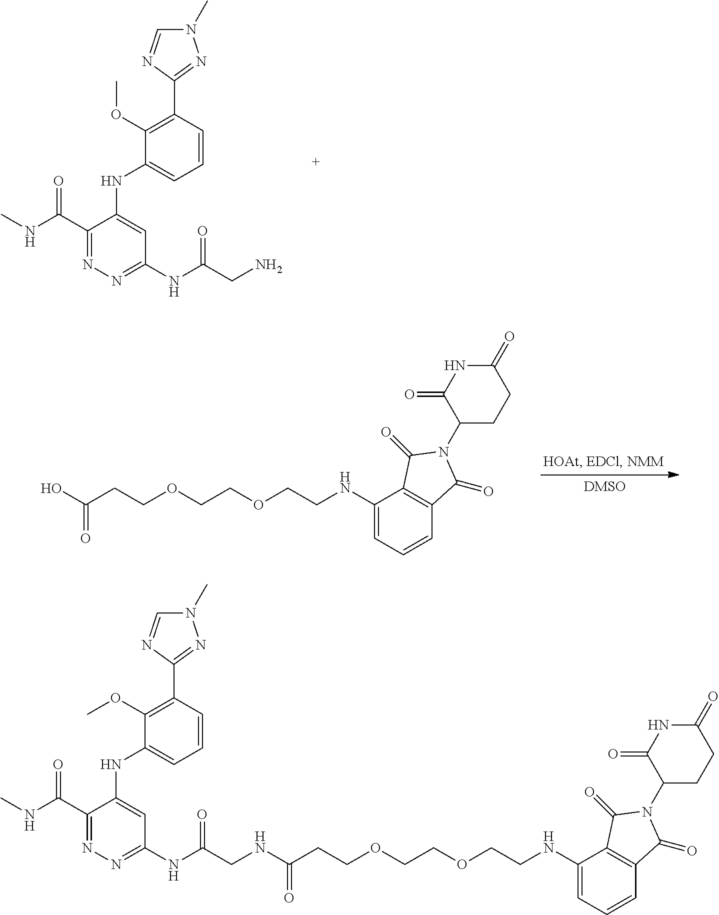 [SMILES: CNC(=O)c1nnc(NC(=O)CN)cc1Nc1cccc(-c2ncn(C)n2)c1OC ; O=C(O)CCOCCOCCNc1cccc2c1C(=O)N(C1CCC(=O)NC1=O)C2=O ; CNC(=O)c1nnc(NC(=O)CNC(=O)CCOCCOCCNc2cccc3c2C(=O)N(C2CCC(=O)NC2=O)C3=O)cc1Nc1cccc(-c2ncn(C)n2)c1OC]

CPD-050 was synthesized following the same procedure for preparing CPD-042 (3.10 mg, yield: 19.3%). MS (ESI) m/z=827.6 [M+H]⁺.

### Example 94: 6-(1-((2-(2,6-Dioxopiperidin-3-yl)-1,3-dioxoisoindolin-4-yl)amino)-12-oxo-3,6,9-trioxa-13-azapentadecan-15-amido)-4-((2-methoxy-3-(1-methyl-1H-1,2,4-triazol-3-yl)phenyl)amino)-N-methylpyridazine-3-carboxamide (CPD-051)

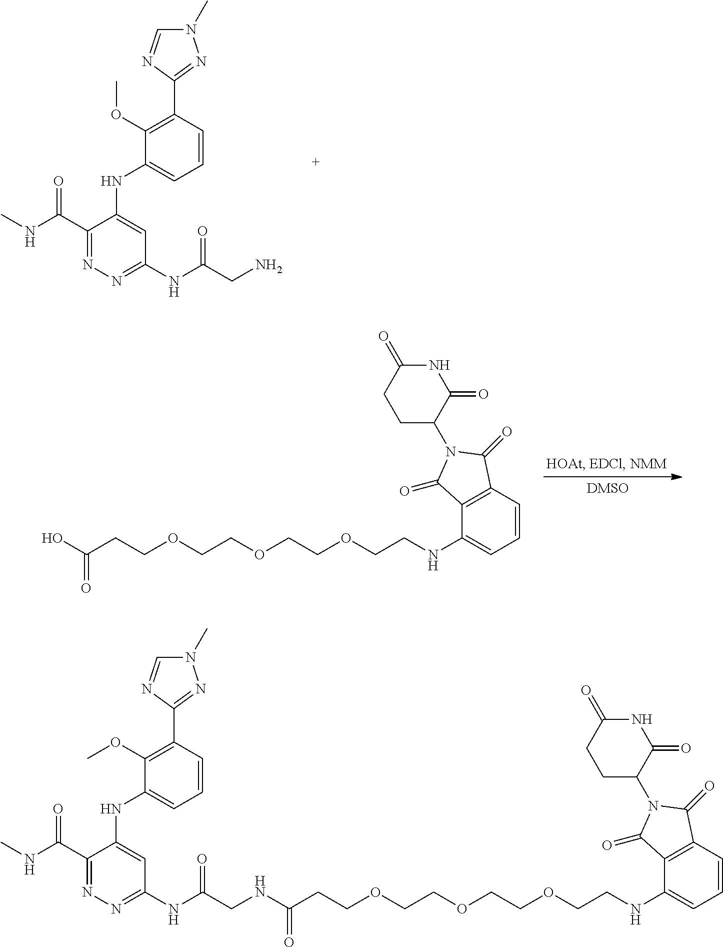 [SMILES: CNC(=O)c1nnc(NC(=O)CN)cc1Nc1cccc(-c2ncn(C)n2)c1OC ; O=C(O)CCOCCOCCOCCNc1cccc2c1C(=O)N(C1CCC(=O)NC1=O)C2=O ; CNC(=O)c1nnc(NC(=O)CNC(=O)CCOCCOCCOCCNc2cccc3c2C(=O)N(C2CCC(=O)NC2=O)C3=O)cc1Nc1cccc(-c2ncn(C)n2)c1OC]

CPD-051 was synthesized following the same procedure for preparing CPD-042 (2.29 mg, yield: 13.6%). MS (ESI) m/z=871.7 [M+H]⁺.

### Example 95: 6-(1-((2-(2,6-Dioxopiperidin-3-yl)-1,3-dioxoisoindolin-4-yl)amino)-15-oxo-3,6,9,12-tetraoxa-16-azaoctadecan-18-amido)-4-((2-methoxy-3-(1-methyl-1H-1,2,4-triazol-3-yl)phenyl)amino)-N-methylpyridazine-3-carboxamide (CPD-052)

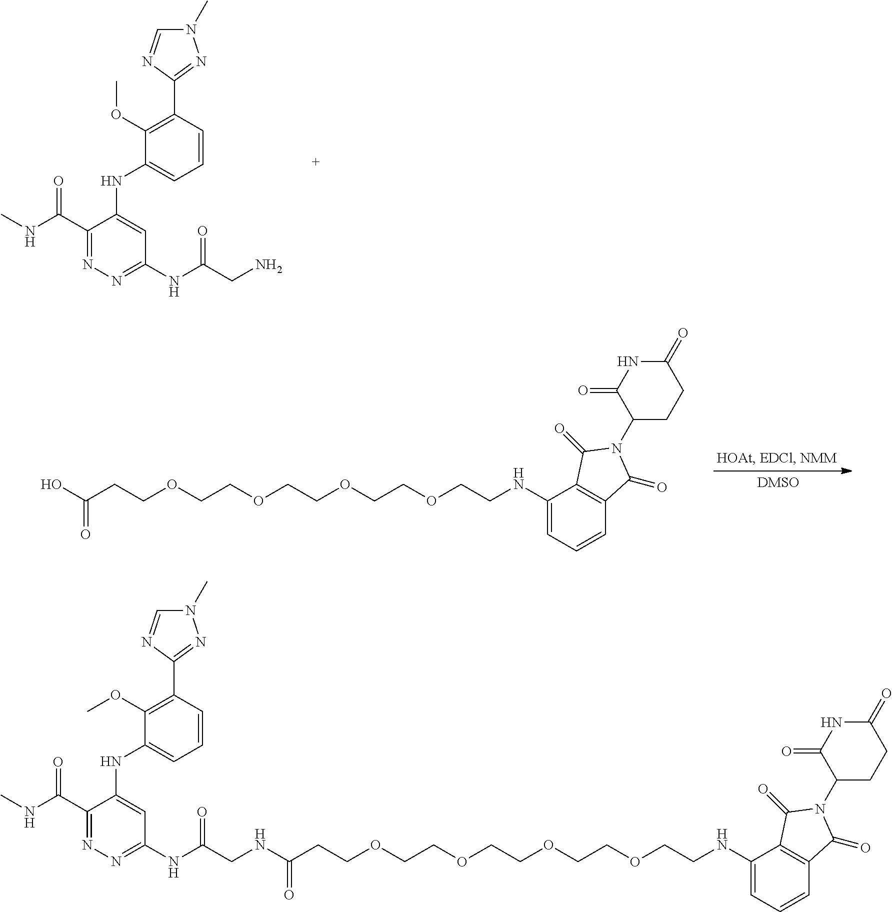 [SMILES: CNC(=O)c1nnc(NC(=O)CN)cc1Nc1cccc(-c2ncn(C)n2)c1OC ; O=C(O)CCOCCOCCOCCOCCNc1cccc2c1C(=O)N(C1CCC(=O)NC1=O)C2=O ; CNC(=O)c1nnc(NC(=O)CNC(=O)CCOCCOCCOCCOCCNc2cccc3c2C(=O)N(C2CCC(=O)NC2=O)C3=O)cc1Nc1cccc(-c2ncn(C)n2)c1OC]

CPD-052 was synthesized following the same procedure for preparing CPD-042 (2.17 mg, yield: 12.2%). MS (ESI) m/z=915.6 [M+H]⁺.

### Example 96: 6-(1-((2-(2,6-Dioxopiperidin-3-yl)-1,3-dioxoisoindolin-4-yl)amino)-18-oxo-3,6,9,12,15-pentaoxa-19-azahenicosan-21-amido)-4-((2-methoxy-3-(1-methyl-1H-1,2,4-triazol-3-yl)phenyl)amino)-N-methylpyridazine-3-carboxamide (CPD-053)

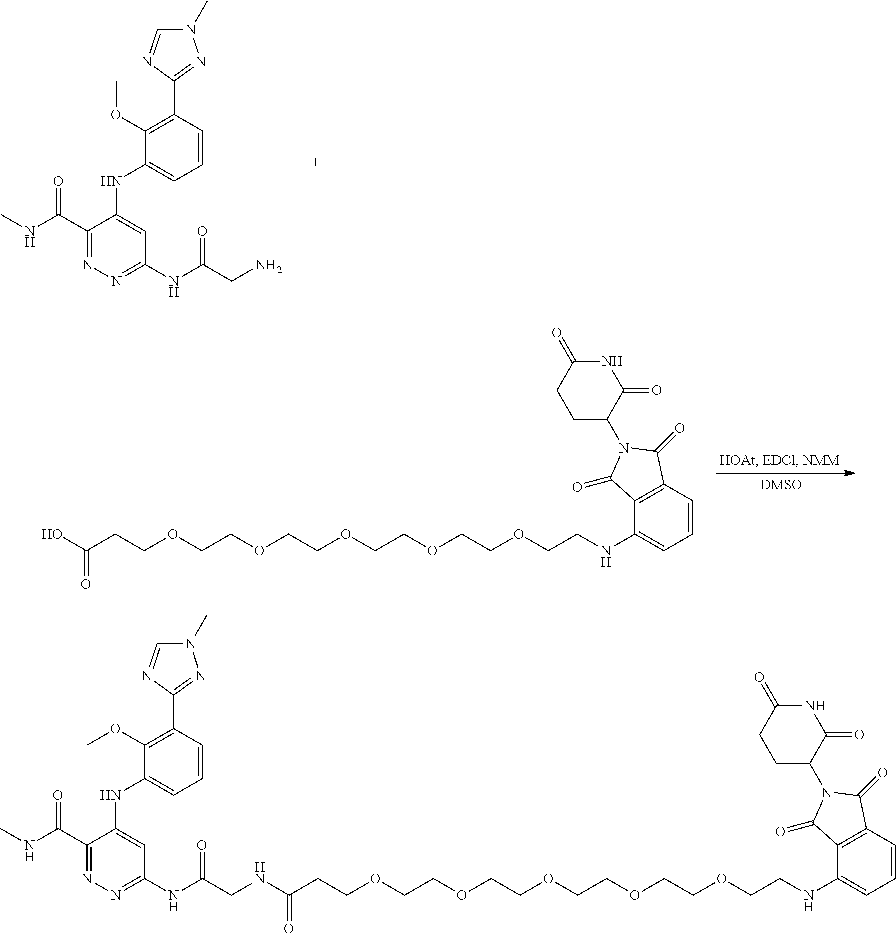 [SMILES: CNC(=O)c1nnc(NC(=O)CN)cc1Nc1cccc(-c2ncn(C)n2)c1OC ; O=C(O)CCOCCOCCOCCOCCOCCNc1cccc2c1C(=O)N(C1CCC(=O)NC1=O)C2=O ; CNC(=O)c1nnc(NC(=O)CNC(=O)CCOCCOCCOCCOCCOCCNc2cccc3c2C(=O)N(C2CCC(=O)NC2=O)C3=O)cc1Nc1cccc(-c2ncn(C)n2)c1OC]

CPD-053 was synthesized following the same procedure for preparing CPD-042 (1.72 mg, yield: 9.2%). MS (ESI) m/z=959.8 [M+H]⁺.

### Example 97: 6-(2-(2-((2-(2,6-Dioxopiperidin-3-yl)-1,3-dioxoisoindolin-5-yl)amino)acetamido)acetamido)-4-((2-methoxy-3-(1-methyl-1H-1,2,4-triazol-3-yl)phenyl)amino)-N-methylpyridazine-3-carboxamide (CPD-054)

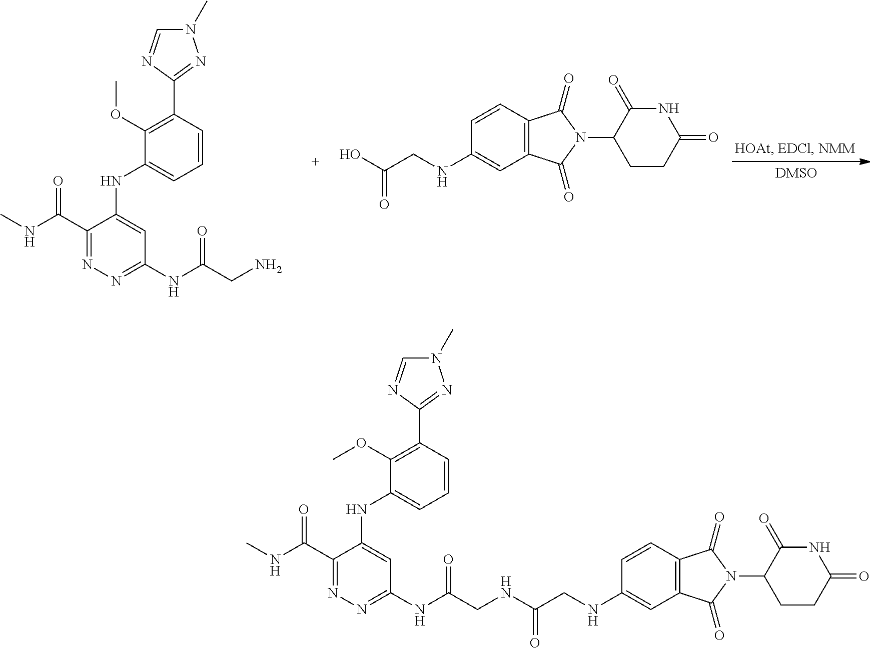 [SMILES: CNC(=O)c1nnc(NC(=O)CN)cc1Nc1cccc(-c2ncn(C)n2)c1OC ; O=C(O)CNc1ccc2c(c1)C(=O)N(C1CCC(=O)NC1=O)C2=O ; CNC(=O)c1nnc(NC(=O)CNC(=O)CNc2ccc3c(c2)C(=O)N(C2CCC(=O)NC2=O)C3=O)cc1Nc1cccc(-c2ncn(C)n2)c1OC]

CPD-054 was synthesized following the same procedure for preparing CPD-042 (2.10 mg, yield: 14.9%). MS (ESI) m/z=725.5 [M+H]⁺.

### Example 98: 6-(2-(3-((2-(2,6-Dioxopiperidin-3-yl)-1,3-dioxoisoindolin-5-yl)amino)propanamido)acetamido)-4-((2-methoxy-3-(1-methyl-1H-1,2,4-triazol-3-yl)phenyl)amino)-N-methylpyridazine-3-carboxamide (CPD-055)

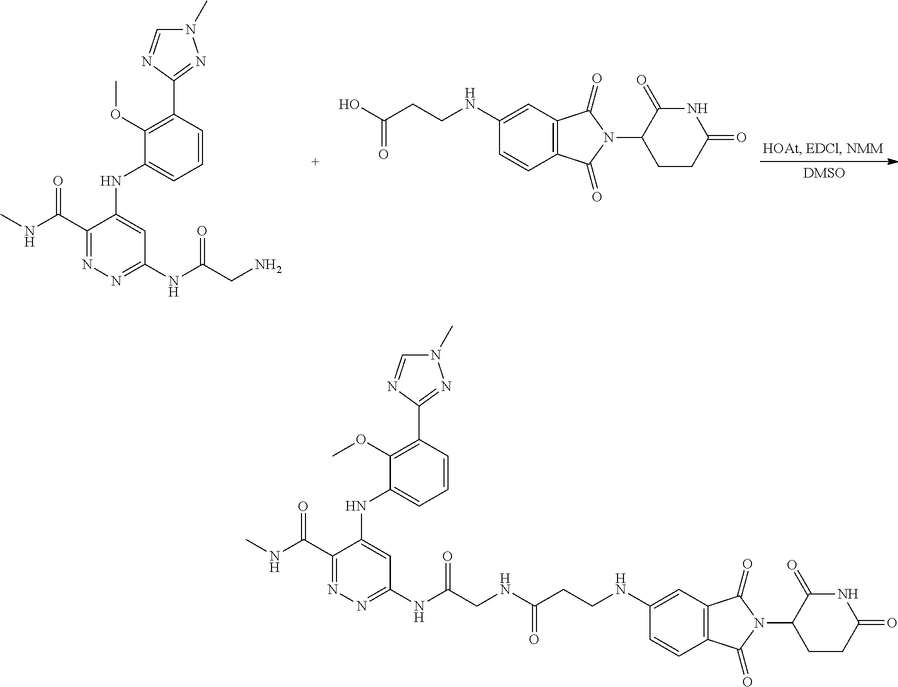 [SMILES: CNC(=O)c1nnc(NC(=O)CN)cc1Nc1cccc(-c2ncn(C)n2)c1OC ; O=C(O)CCNc1ccc2c(c1)C(=O)N(C1CCC(=O)NC1=O)C2=O ; CNC(=O)c1nnc(NC(=O)CNC(=O)CCNc2ccc3c(c2)C(=O)N(C2CCC(=O)NC2=O)C3=O)cc1Nc1cccc(-c2ncn(C)n2)c1OC]

CPD-055 was synthesized following the same procedure for preparing CPD-042 (3.01 g, yield: 20.9%). MS (ESI) m/z=739.5 [M+H]⁺.

### Example 99: 6-(2-(4-((2-(2,6-Dioxopiperidin-3-yl)-1,3-dioxoisoindolin-5-yl)amino)butanamido)acetamido)-4-((2-methoxy-3-(1-methyl-1H-1,2,4-triazol-3-yl)phenyl)amino)-N-methylpyridazine-3-carboxamide (CPD-056)

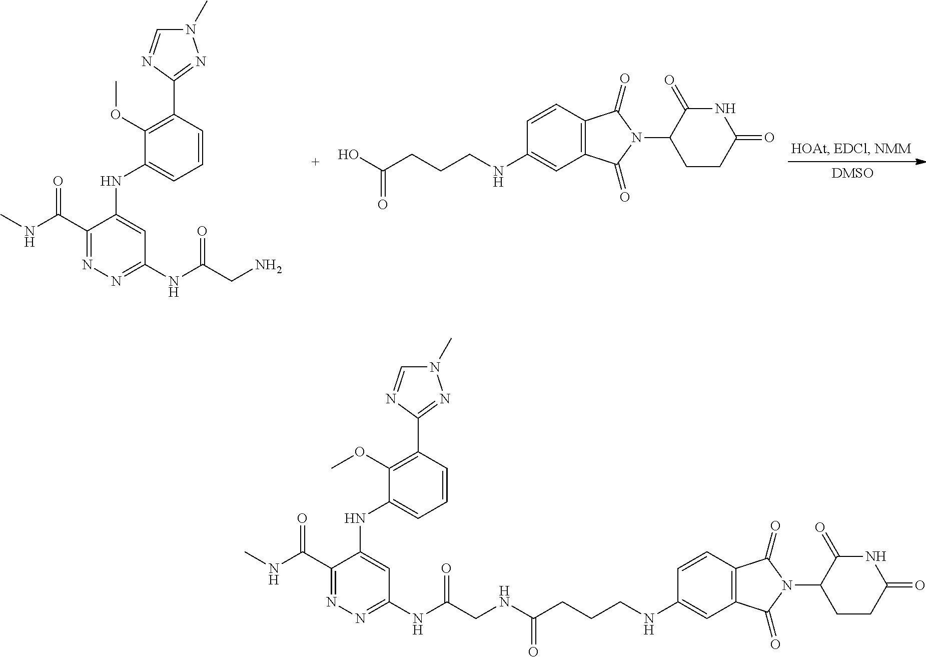 [SMILES: CNC(=O)c1nnc(NC(=O)CN)cc1Nc1cccc(-c2ncn(C)n2)c1OC ; O=C(O)CCCNc1ccc2c(c1)C(=O)N(C1CCC(=O)NC1=O)C2=O ; CNC(=O)c1nnc(NC(=O)CNC(=O)CCCNc2ccc3c(c2)C(=O)N(C2CCC(=O)NC2=O)C3=O)cc1Nc1cccc(-c2ncn(C)n2)c1OC]

CPD-056 was synthesized following the same procedure for preparing CPD-042 (2.20 mg, yield: 15.1%). MS (ESI) m/z=753.6 [M+H]⁺.

### Example 100: 6-(2-(5-((2-(2,6-Dioxopiperidin-3-yl)-1,3-dioxoisoindolin-5-yl)amino)pentanamido)acetamido)-4-((2-methoxy-3-(1-methyl-1H-1,2,4-triazol-3-yl)phenyl)amino)-N-methylpyridazine-3-carboxamide (CPD-057)

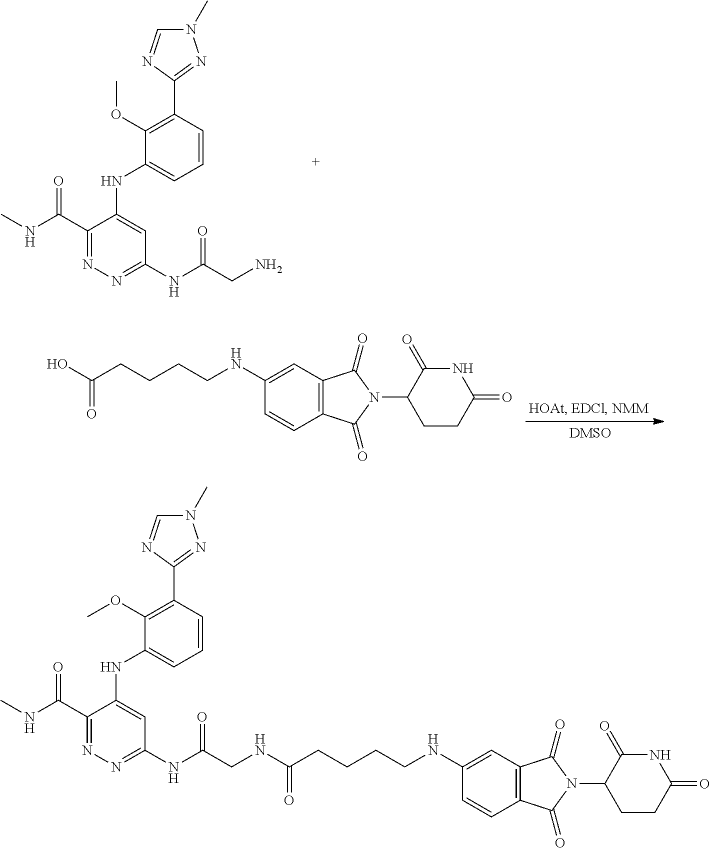 [SMILES: CNC(=O)c1nnc(NC(=O)CN)cc1Nc1cccc(-c2ncn(C)n2)c1OC ; O=C(O)CCCCNc1ccc2c(c1)C(=O)N(C1CCC(=O)NC1=O)C2=O ; CNC(=O)c1nnc(NC(=O)CNC(=O)CCCCNc2ccc3c(c2)C(=O)N(C2CCC(=O)NC2=O)C3=O)cc1Nc1cccc(-c2ncn(C)n2)c1OC]

CPD-057 was synthesized following the same procedure for preparing CPD-042 (1.50 mg, yield: 10.1%). MS (ESI) m/z=767.5 [M+H]⁺.

### Example 101: 6-(2-(6-((2-(2,6-Dioxopiperidin-3-yl)-1,3-dioxoisoindolin-5-yl)amino)hexanamido)acetamido)-4-((2-methoxy-3-(1-methyl-1H-1,2,4-triazol-3-yl)phenyl)amino)-N-methylpyridazine-3-carboxamide (CPD-058)

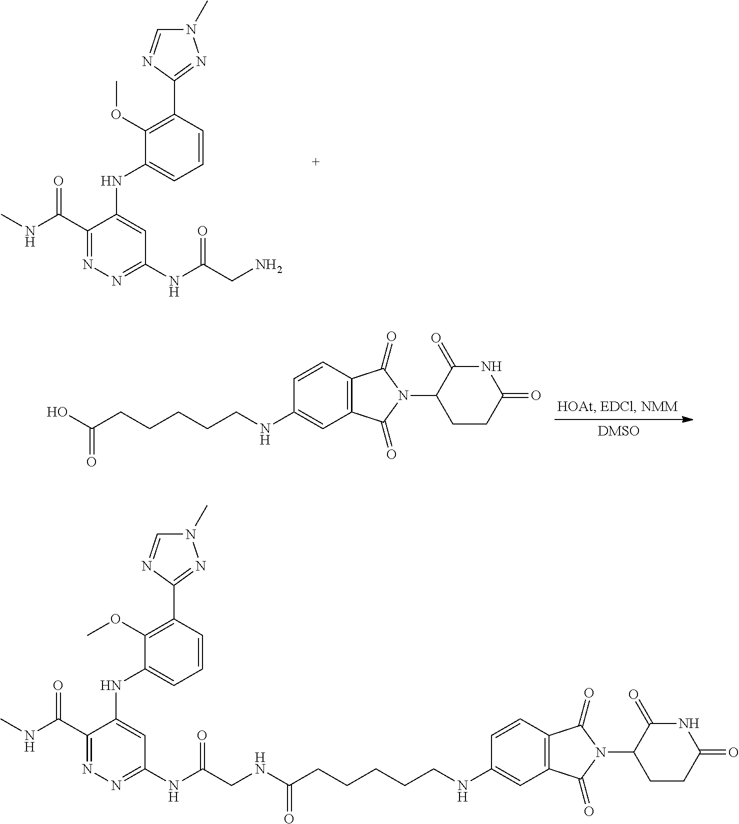 [SMILES: CNC(=O)c1nnc(NC(=O)CN)cc1Nc1cccc(-c2ncn(C)n2)c1OC ; O=C(O)CCCCCNc1ccc2c(c1)C(=O)N(C1CCC(=O)NC1=O)C2=O ; CNC(=O)c1nnc(NC(=O)CNC(=O)CCCCCNc2ccc3c(c2)C(=O)N(C2CCC(=O)NC2=O)C3=O)cc1Nc1cccc(-c2ncn(C)n2)c1OC]

CPD-058 was synthesized following the same procedure for preparing CPD-042 (1.71 mg, yield: 11.3%). MS (ESI) m/z=781.6 [M+H]⁺.

### Example 102: 6-(2-(7-((2-(2,6-Dioxopiperidin-3-yl)-1,3-dioxoisoindolin-5-yl)amino)heptanamido)acetamido)-4-((2-methoxy-3-(1-methyl-1H-1,2,4-triazol-3-yl)phenyl)amino)-N-methylpyridazine-3-carboxamide (CPD-059)

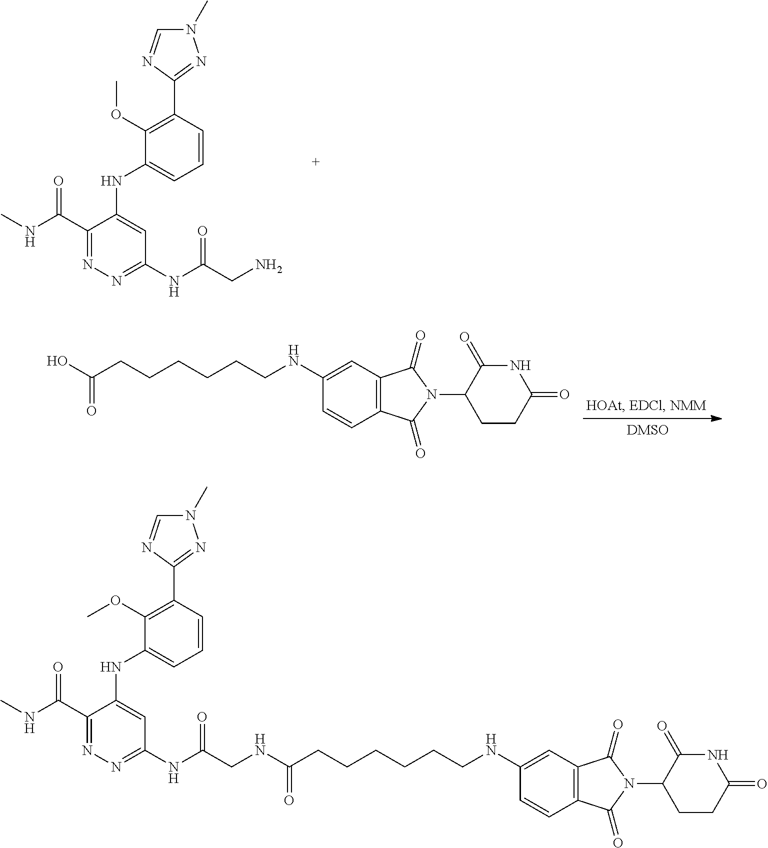 [SMILES: CNC(=O)c1nnc(NC(=O)CN)cc1Nc1cccc(-c2ncn(C)n2)c1OC ; O=C(O)CCCCCCNc1ccc2c(c1)C(=O)N(C1CCC(=O)NC1=O)C2=O ; CNC(=O)c1nnc(NC(=O)CNC(=O)CCCCCCNc2ccc3c(c2)C(=O)N(C2CCC(=O)NC2=O)C3=O)cc1Nc1cccc(-c2ncn(C)n2)c1OC]

CPD-059 was synthesized following the same procedure for preparing CPD-042 (1.47 mg, yield: 9.5%). MS (ESI) m/z=795.6 [M+H]⁺.

### Example 103: 6-(2-(8-((2-(2,6-Dioxopiperidin-3-yl)-1,3-dioxoisoindolin-5-yl)amino)octanamido)acetamido)-4-((2-methoxy-3-(1-methyl-1H-1,2,4-triazol-3-yl)phenyl)amino)-N-methylpyridazine-3-carboxamide (CPD-060)

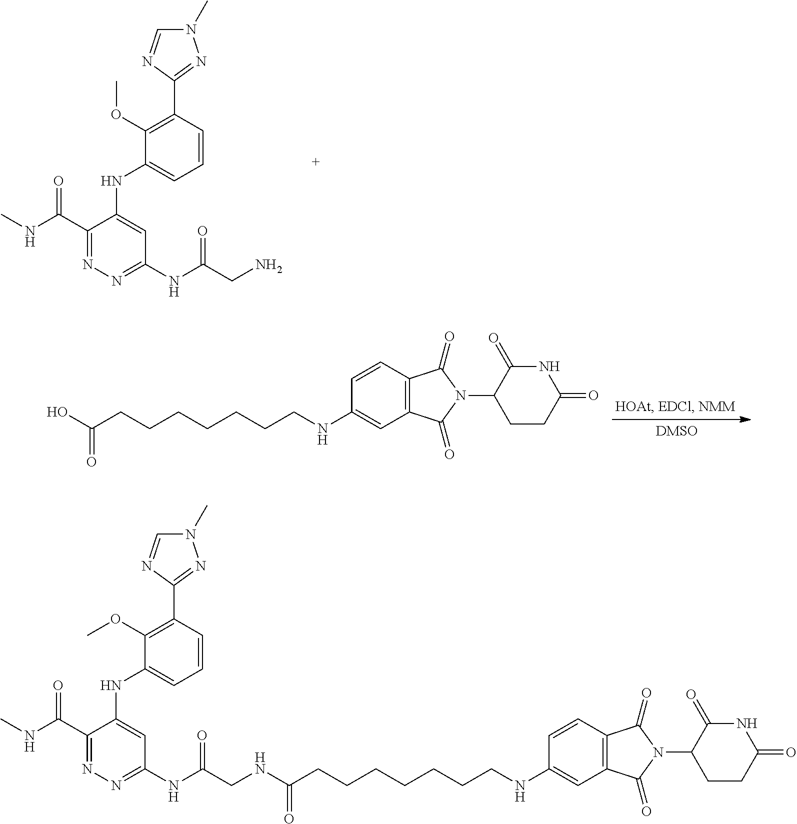 [SMILES: CNC(=O)c1nnc(NC(=O)CN)cc1Nc1cccc(-c2ncn(C)n2)c1OC ; O=C(O)CCCCCCCNc1ccc2c(c1)C(=O)N(C1CCC(=O)NC1=O)C2=O ; CNC(=O)c1nnc(NC(=O)CNC(=O)CCCCCCCNc2ccc3c(c2)C(=O)N(C2CCC(=O)NC2=O)C3=O)cc1Nc1cccc(-c2ncn(C)n2)c1OC]

CPD-060 was synthesized following the same procedure for preparing CPD-042 (1.64 mg, yield: 10.4%). MS (ESI) m/z=809.7 [M+H]⁺.

### Example 104: 6-(2-(3-(2-((2-(2,6-Dioxopiperidin-3-yl)-1,3-dioxoisoindolin-5-yl)amino)ethoxy)propanamido)acetamido)-4-((2-methoxy-3-(1-methyl-1H-1,2,4-triazol-3-yl)phenyl)amino)-N-methylpyridazine-3-carboxamide (CPD-061)

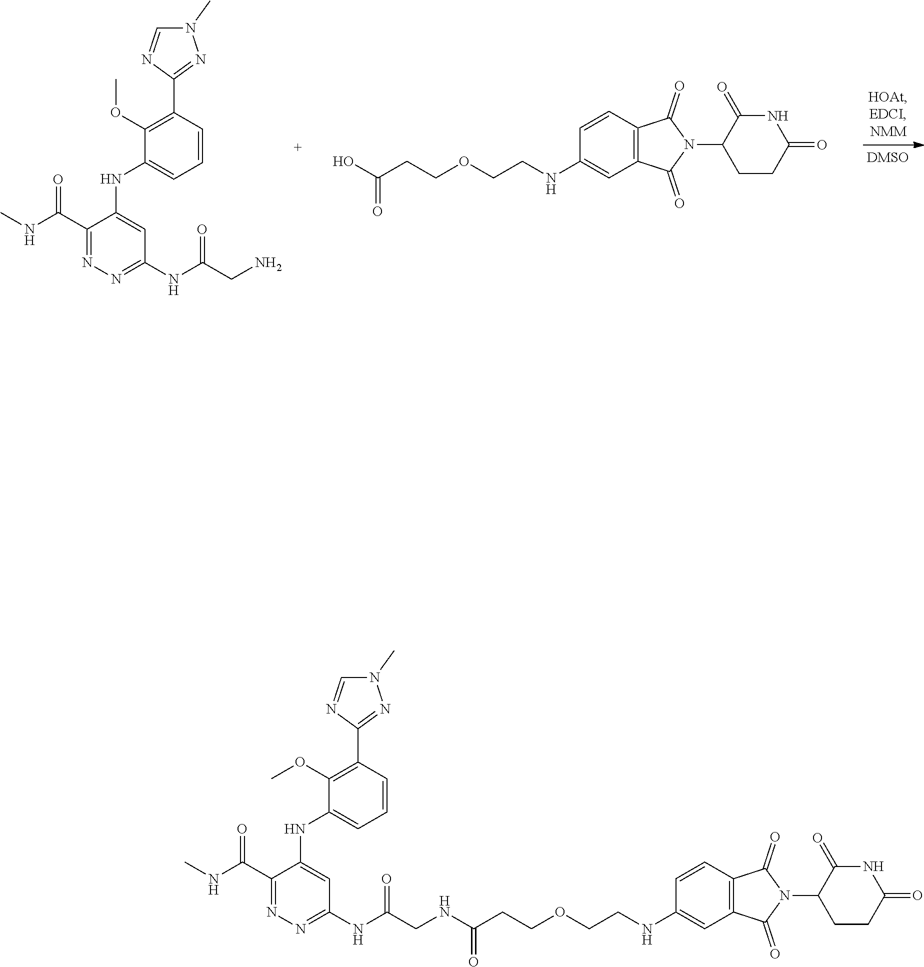 [SMILES: CNC(=O)c1nnc(NC(=O)CN)cc1Nc1cccc(-c2ncn(C)n2)c1OC ; O=C(O)CCOCCNc1ccc2c(c1)C(=O)N(C1CCC(=O)NC1=O)C2=O ; CNC(=O)c1nnc(NC(=O)CNC(=O)CCOCCNc2ccc3c(c2)C(=O)N(C2CCC(=O)NC2=O)C3=O)cc1Nc1cccc(-c2ncn(C)n2)c1OC]

CPD-061 was synthesized following the same procedure for preparing CPD-042 (2.46 mg, yield: 16.2%). MS (ESI) m/z=783.6 [M+H]⁺.

### Example 105: 6-(2-(3-(2-(2-((2-(2,6-Dioxopiperidin-3-yl)-1,3-dioxoisoindolin-5-yl)amino)ethoxy)ethoxy)propanamido)acetamido)-4-((2-methoxy-3-(1-methyl-1H-1,2,4-triazol-3-yl)phenyl)amino)-N-methylpyridazine-3-carboxamide (CPD-062)

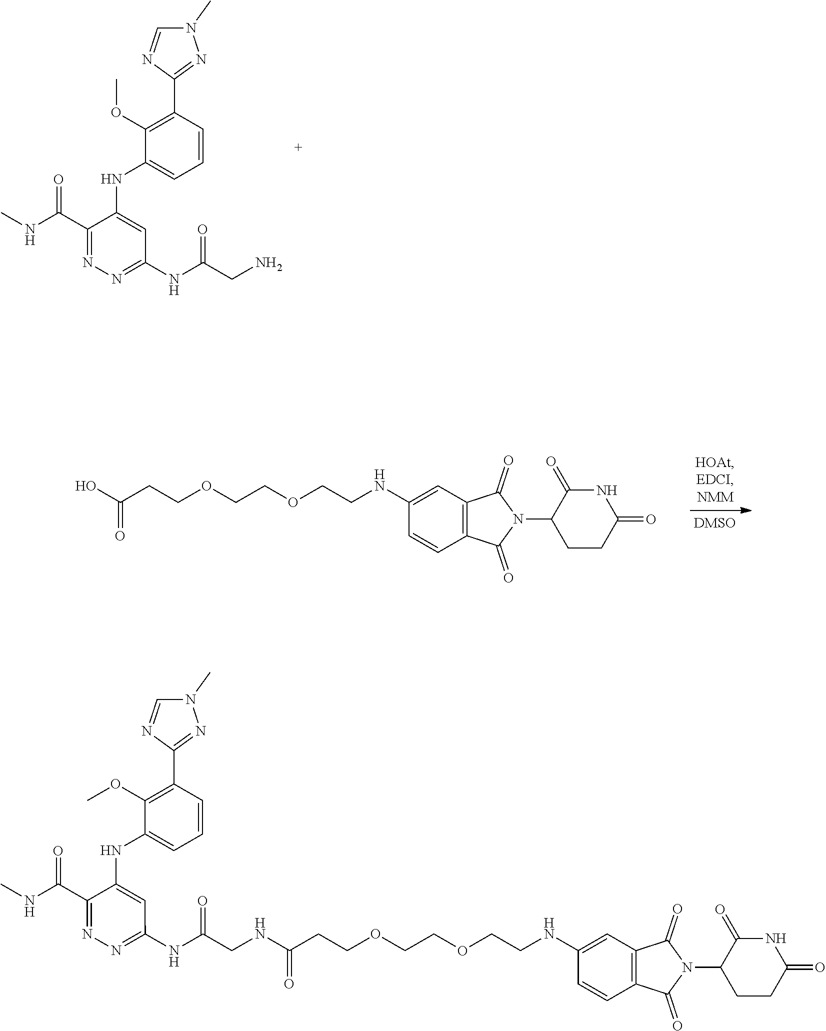 [SMILES: CNC(=O)c1nnc(NC(=O)CN)cc1Nc1cccc(-c2ncn(C)n2)c1OC ; O=C(O)CCOCCOCCNc1ccc2c(c1)C(=O)N(C1CCC(=O)NC1=O)C2=O ; CNC(=O)c1nnc(NC(=O)CNC(=O)CCOCCOCCNc2ccc3c(c2)C(=O)N(C2CCC(=O)NC2=O)C3=O)cc1Nc1cccc(-c2ncn(C)n2)c1OC]

CPD-062 was synthesized following the same procedure for preparing CPD-042 (2.79 mg, yield: 17.4%). MS (ESI) m/z=827.6 [M+H]⁺.

### Example 106: 6-(1-((2-(2,6-Dioxopiperidin-3-yl)-1,3-dioxoisoindolin-5-yl)amino)-12-oxo-3,6,9-trioxa-13-azapentadecan-15-amido)-4-((2-methoxy-3-(1-methyl-1H-1,2,4-triazol-3-yl)phenyl)amino)-N-methylpyridazine-3-carboxamide (CPD-063)

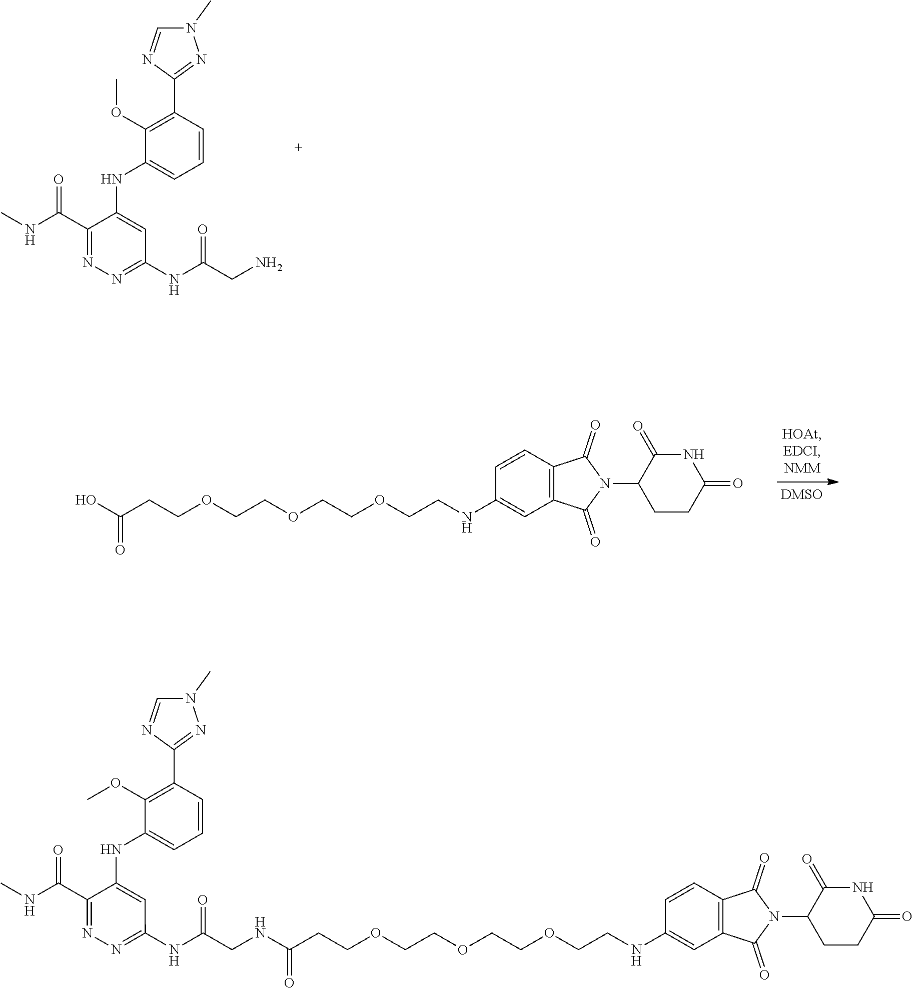 [SMILES: CNC(=O)c1nnc(NC(=O)CN)cc1Nc1cccc(-c2ncn(C)n2)c1OC ; O=C(O)CCOCCOCCOCCNc1ccc2c(c1)C(=O)N(C1CCC(=O)NC1=O)C2=O ; CNC(=O)c1nnc(NC(=O)CNC(=O)CCOCCOCCOCCNc2ccc3c(c2)C(=O)N(C2CCC(=O)NC2=O)C3=O)cc1Nc1cccc(-c2ncn(C)n2)c1OC]

CPD-063 was synthesized following the same procedure for preparing CPD-042 (2.04 mg, yield: 12%). MS (ESI) m/z=871.7 [M+H]⁺.

### Example 107: 6-(1-((2-(2,6-Dioxopiperidin-3-yl)-1,3-dioxoisoindolin-5-yl)amino)-15-oxo-3,6,9,12-tetraoxa-16-azaoctadecan-18-amido)-4-((2-methoxy-3-(1-methyl-1H-1,2,4-triazol-3-yl)phenyl)amino)-N-methylpyridazine-3-carboxamide (CPD-064)

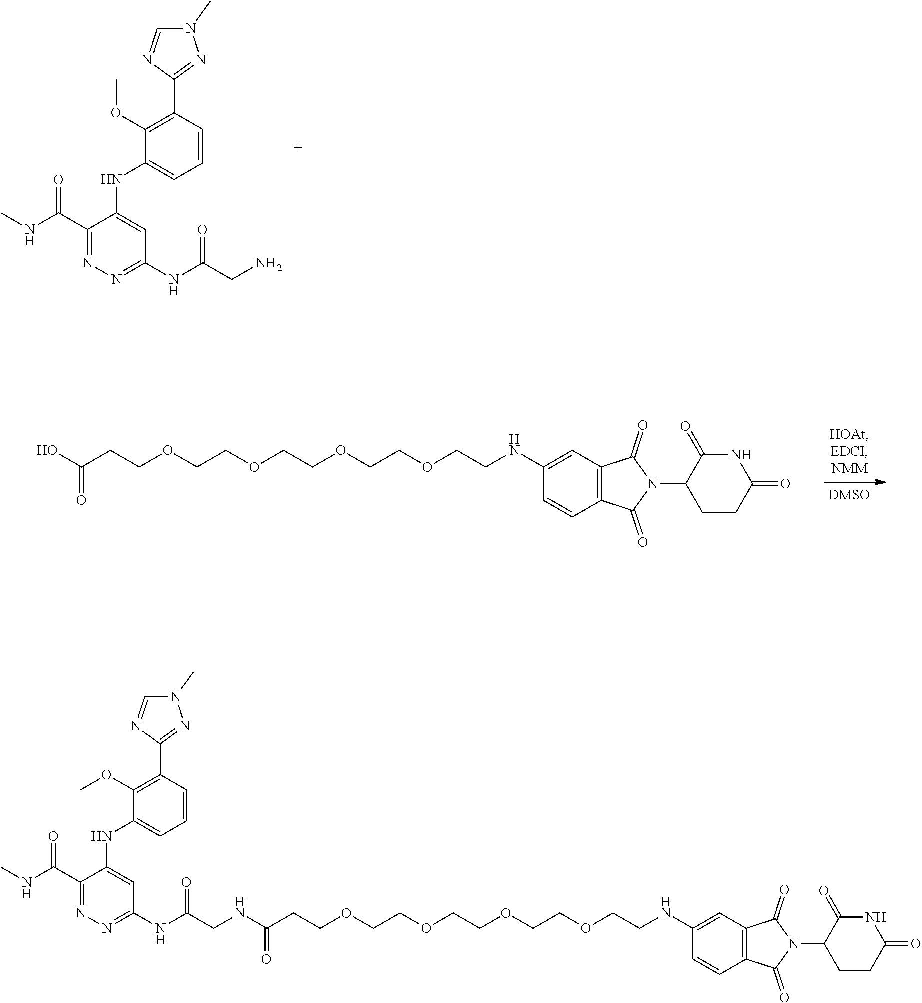 [SMILES: CNC(=O)c1nnc(NC(=O)CN)cc1Nc1cccc(-c2ncn(C)n2)c1OC ; O=C(O)CCOCCOCCOCCOCCNc1ccc2c(c1)C(=O)N(C1CCC(=O)NC1=O)C2=O ; CNC(=O)c1nnc(NC(=O)CNC(=O)CCOCCOCCOCCOCCNc2ccc3c(c2)C(=O)N(C2CCC(=O)NC2=O)C3=O)cc1Nc1cccc(-c2ncn(C)n2)c1OC]

CPD-064 was synthesized following the same procedure for preparing CPD-042 (2.87 mg, yield: 16.1%). MS (ESI) m/z=915.7 [M+H]⁺.

### Example 108: 6-(1-((2-(2,6-Dioxopiperidin-3-yl)-1,3-dioxoisoindolin-5-yl)amino)-18-oxo-3,6,9,12,15-pentaoxa-19-azahenicosan-21-amido)-4-((2-methoxy-3-(1-methyl-1H-1,2,4-triazol-3-yl)phenyl)amino)-N-methylpyridazine-3-carboxamide (CPD-065)

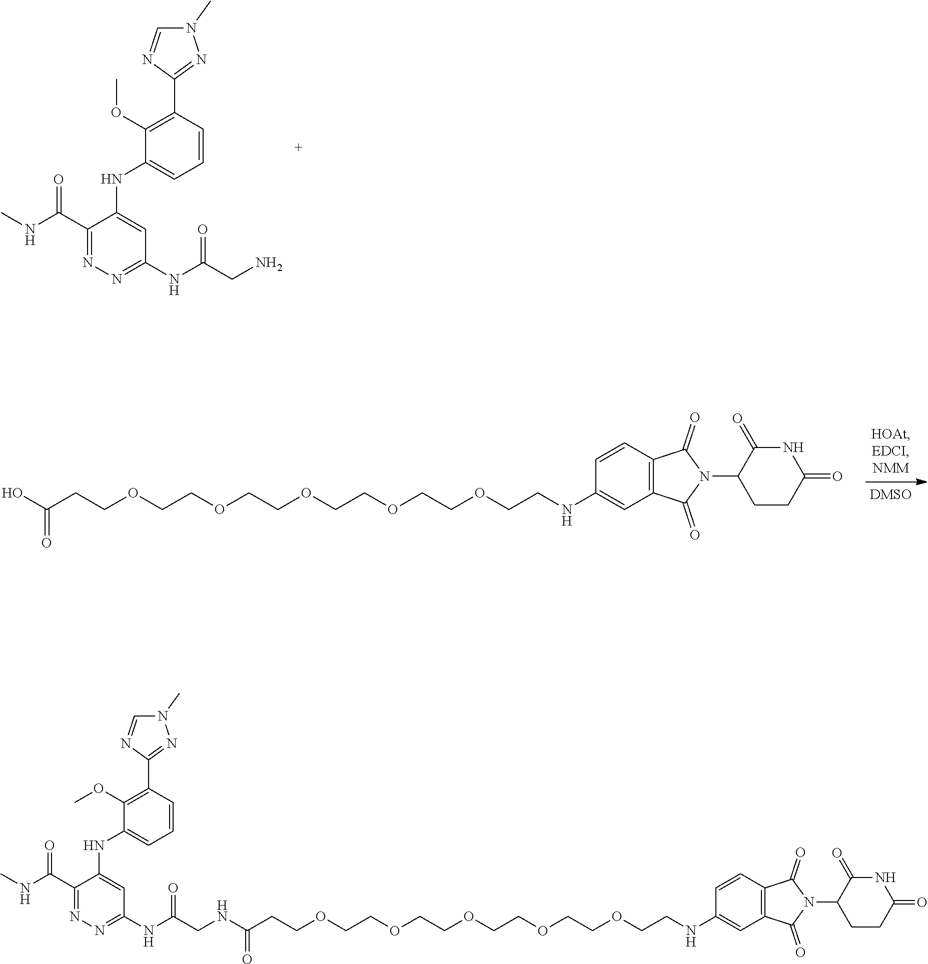 [SMILES: CNC(=O)c1nnc(NC(=O)CN)cc1Nc1cccc(-c2ncn(C)n2)c1OC ; O=C(O)CCOCCOCCOCCOCCOCCNc1ccc2c(c1)C(=O)N(C1CCC(=O)NC1=O)C2=O ; CNC(=O)c1nnc(NC(=O)CNC(=O)CCOCCOCCOCCOCCOCCNc2ccc3c(c2)C(=O)N(C2CCC(=O)NC2=O)C3=O)cc1Nc1cccc(-c2ncn(C)n2)c1OC]

CPD-065 was synthesized following the same procedure for preparing CPD-042 (3.17 mg, yield: 17%). MS (ESI) m/z=959.8 [M+H]⁺.

### Example 109: 6-(Cyclopropanecarboxamido)-4-((3-(1-(2-(2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)acetamido)ethyl)-1H-1,2,4-triazol-3-yl)-2-methoxyphenyl)amino)-N-methylpyridazine-3-carboxamide (CPD-066)

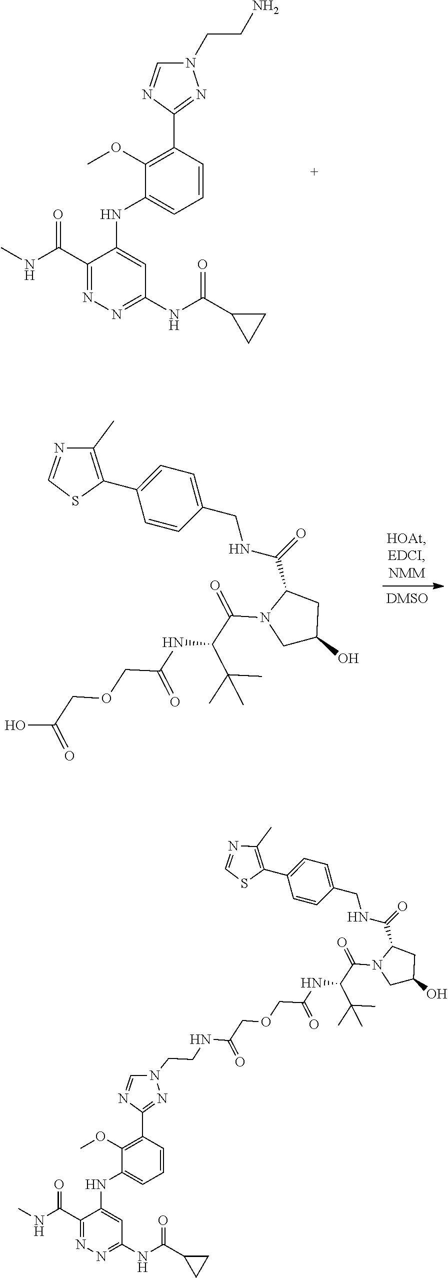 [SMILES: CNC(=O)c1nnc(NC(=O)C2CC2)cc1Nc1cccc(-c2ncn(CCN)n2)c1OC ; Cc1ncsc1-c1ccc(CNC(=O)[C@@H]2C[C@@H](O)CN2C(=O)[C@@H](NC(=O)COCC(=O)O)C(C)(C)C)cc1 ; CNC(=O)c1nnc(NC(=O)C2CC2)cc1Nc1cccc(-c2ncn(CCNC(=O)COCC(=O)N[C@H](C(=O)N3C[C@H](O)C[C@H]3C(=O)NCc3ccc(-c4scnc4C)cc3)C(C)(C)C)n2)c1OC]

CPD-066 was synthesized following the same procedure for preparing CPD-042 (3.95 mg, yield: 36.4%). MS (ESI) m/z=980.8 [M+H]⁺.

### Example 110: 6-(Cyclopropanecarboxamido)-4-((3-(1-(2-(3-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)propanamido)ethyl)-1H-1,2,4-triazol-3-yl)-2-methoxyphenyl)amino)-N-methylpyridazine-3-carboxamide (CPD-067)

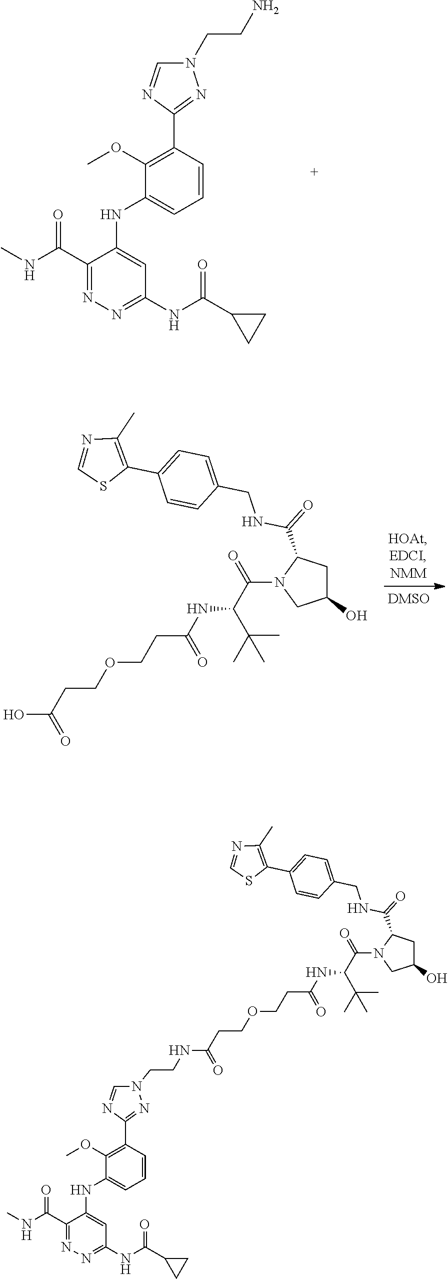 [SMILES: CNC(=O)c1nnc(NC(=O)C2CC2)cc1Nc1cccc(-c2ncn(CCN)n2)c1OC ; Cc1ncsc1-c1ccc(CNC(=O)[C@@H]2C[C@@H](O)CN2C(=O)[C@@H](NC(=O)CCOCCC(=O)O)C(C)(C)C)cc1 ; CNC(=O)c1nnc(NC(=O)C2CC2)cc1Nc1cccc(-c2ncn(CCNC(=O)CCOCCC(=O)N[C@H](C(=O)N3C[C@H](O)C[C@H]3C(=O)NCc3ccc(-c4scnc4C)cc3)C(C)(C)C)n2)c1OC]

CPD-067 was synthesized following the same procedure for preparing CPD-042 (3.59 mg, yield: 32.1%). MS (ESI) m/z=1008.7 [M+H]⁺.

### Example 111: 6-(Cyclopropanecarboxamido)-4-((3-(1-((S)-13-((2S,4R)-4-hydroxy-2-((4-(4-methylthiazol-5-yl)benzyl)carbamoyl)pyrrolidine-1-carbonyl)-14,14-dimethyl-4,11-dioxo-6,9-dioxa-3,12-diazapentadecyl)-1H-1,2,4-triazol-3-yl)-2-methoxyphenyl)amino)-N-methylpyridazine-3-carboxamide (CPD-068)

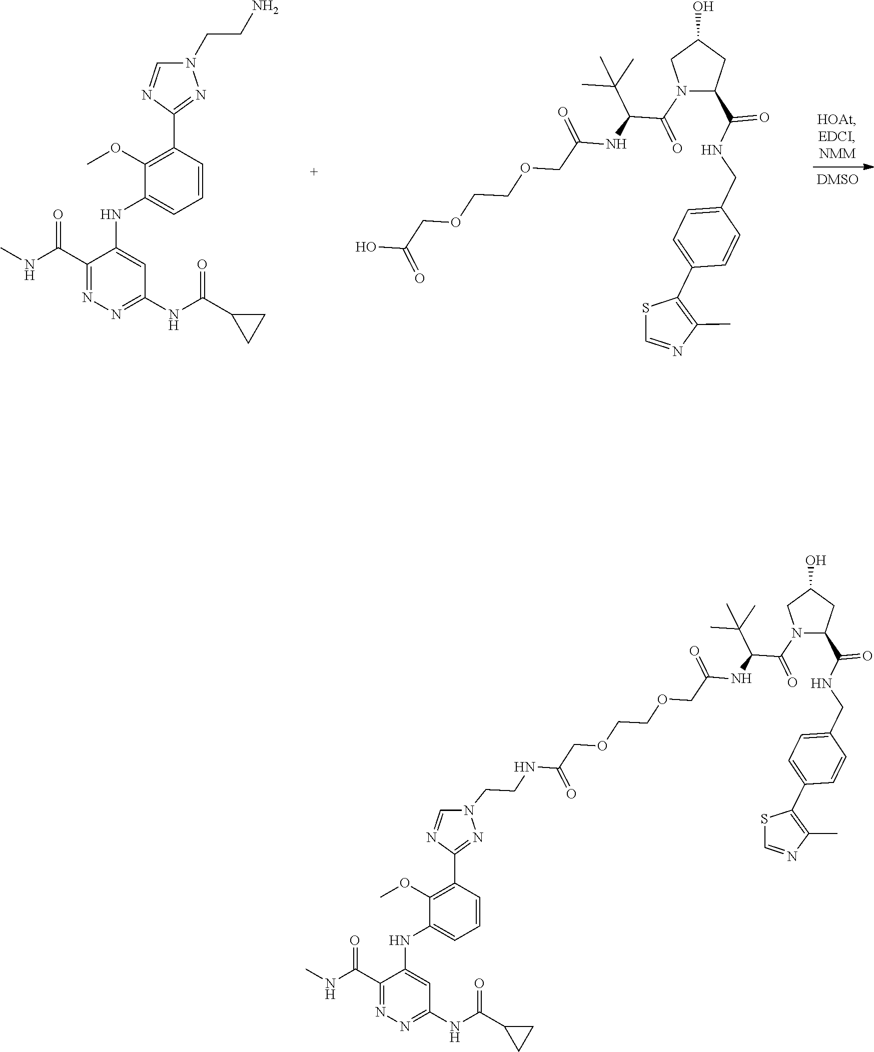 [SMILES: CNC(=O)c1nnc(NC(=O)C2CC2)cc1Nc1cccc(-c2ncn(CCN)n2)c1OC ; Cc1ncsc1-c1ccc(CNC(=O)[C@@H]2C[C@@H](O)CN2C(=O)[C@@H](NC(=O)COCCOCC(=O)O)C(C)(C)C)cc1 ; CNC(=O)c1nnc(NC(=O)C2CC2)cc1Nc1cccc(-c2ncn(CCNC(=O)COCCOCC(=O)N[C@H](C(=O)N3C[C@H](O)C[C@H]3C(=O)NCc3ccc(-c4scnc4C)cc3)C(C)(C)C)n2)c1OC]

CPD-068 was synthesized following the same procedure for preparing CPD-042 (3.61 mg, yield: 31.8%). MS (ESI) m/z=1024.7 [M+H]⁺.

### Example 112: 6-(Cyclopropanecarboxamido)-4-((3-(1-((S)-15-((2S,4R)-4-hydroxy-2-((4-(4-methylthiazol-5-yl)benzyl)carbamoyl)pyrrolidine-1-carbonyl)-16,16-dimethyl-4,13-dioxo-7,10-dioxa-3,14-diazaheptadecyl)-1H-1,2,4-triazol-3-yl)-2-methoxyphenyl)amino)-N-methylpyridazine-3-carboxamide (CPD-069)

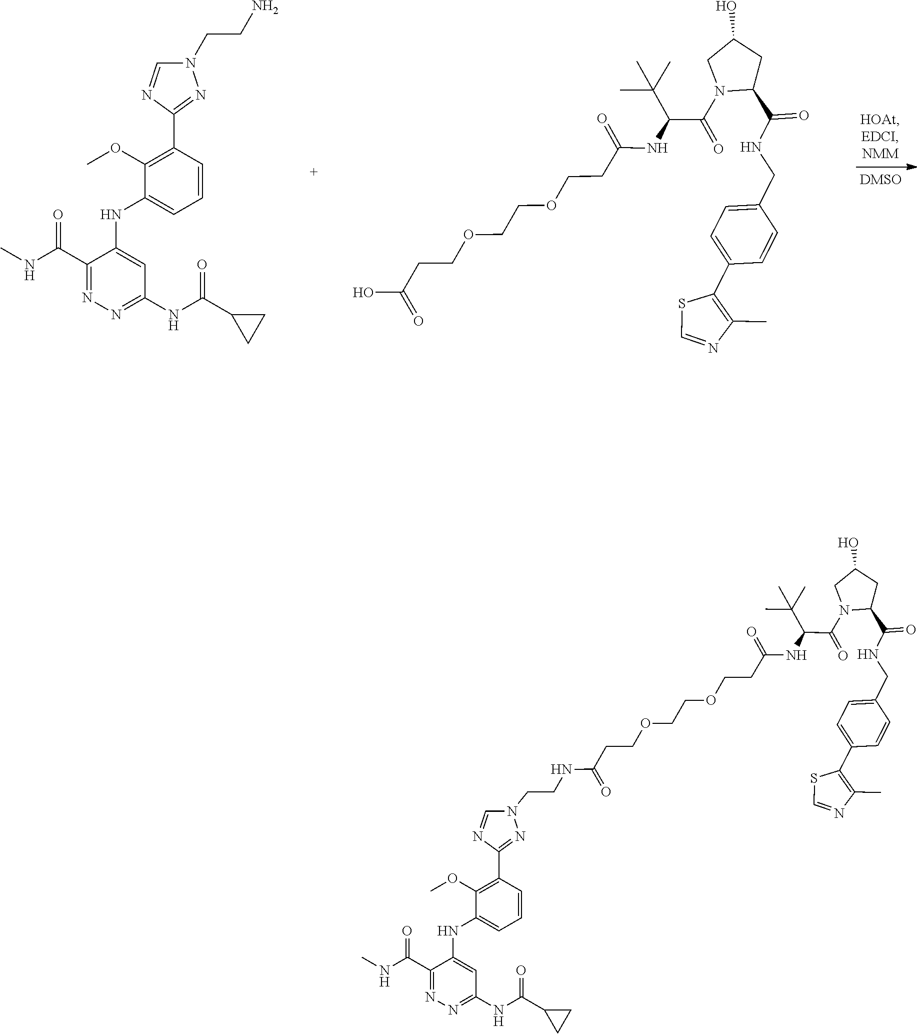 [SMILES: CNC(=O)c1nnc(NC(=O)C2CC2)cc1Nc1cccc(-c2ncn(CCN)n2)c1OC ; Cc1ncsc1-c1ccc(CNC(=O)[C@@H]2C[C@@H](O)CN2C(=O)[C@@H](NC(=O)CCOCCOCCC(=O)O)C(C)(C)C)cc1 ; CNC(=O)c1nnc(NC(=O)C2CC2)cc1Nc1cccc(-c2ncn(CCNC(=O)CCOCCOCCC(=O)N[C@H](C(=O)N3C[C@H](O)C[C@H]3C(=O)NCc3ccc(-c4scnc4C)cc3)C(C)(C)C)n2)c1OC]

CPD-069 was synthesized following the same procedure for preparing CPD-042 (4.37 mg, yield: 37.5%). MS (ESI) m/z=1052.7 [M+H]⁺.

### Example 113: 6-(Cyclopropanecarboxamido)-4-((3-(1-((S)-16-((2S,4R)-4-hydroxy-2-((4-(4-methylthiazol-5-yl)benzyl)carbamoyl)pyrrolidine-1-carbonyl)-17,17-dimethyl-4,14-dioxo-6,9,12-trioxa-3,15-diazaoctadecyl)-1H-1,2,4-triazol-3-yl)-2-methoxyphenyl)amino)-N-methylpyridazine-3-carboxamide (CPD-070)

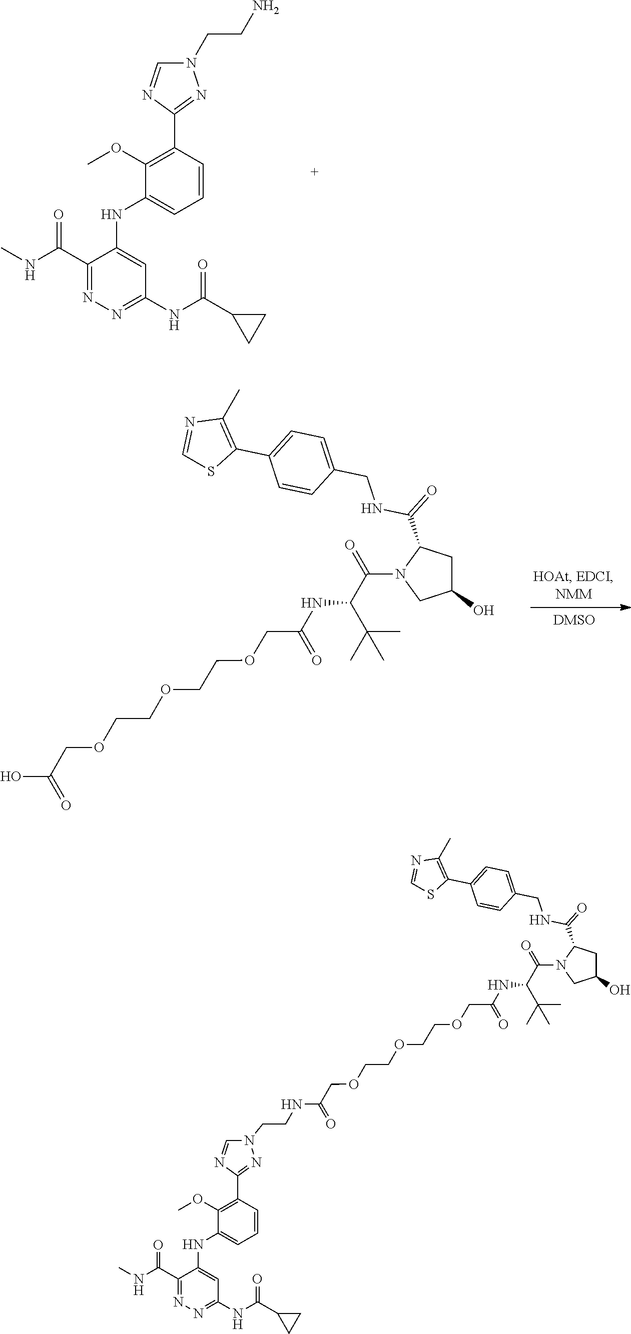 [SMILES: CNC(=O)c1nnc(NC(=O)C2CC2)cc1Nc1cccc(-c2ncn(CCN)n2)c1OC ; Cc1ncsc1-c1ccc(CNC(=O)[C@@H]2C[C@@H](O)CN2C(=O)[C@@H](NC(=O)COCCOCCOCC(=O)O)C(C)(C)C)cc1 ; CNC(=O)c1nnc(NC(=O)C2CC2)cc1Nc1cccc(-c2ncn(CCNC(=O)COCCOCCOCC(=O)N[C@H](C(=O)N3C[C@H](O)C[C@H]3C(=O)NCc3ccc(-c4scnc4C)cc3)C(C)(C)C)n2)c1OC]

CPD-070 was synthesized following the same procedure for preparing CPD-042 (2.96 mg, yield: 25%). MS (ESI) m/z=1068.7 [M+H]⁺.

### Example 114: 6-(Cyclopropanecarboxamido)-4-((3-(1-((5)-18-((2S,4R)-4-hydroxy-2-((4-(4-methylthiazol-5-yl)benzyl)carbamoyl)pyrrolidine-1-carbonyl)-19,19-dimethyl-4,16-dioxo-7,10,13-trioxa-3,17-diazaicosyl)-1H-1,2,4-triazol-3-yl)-2-methoxyphenyl)amino)-N-methylpyridazine-3-carboxamide (CPD-071)

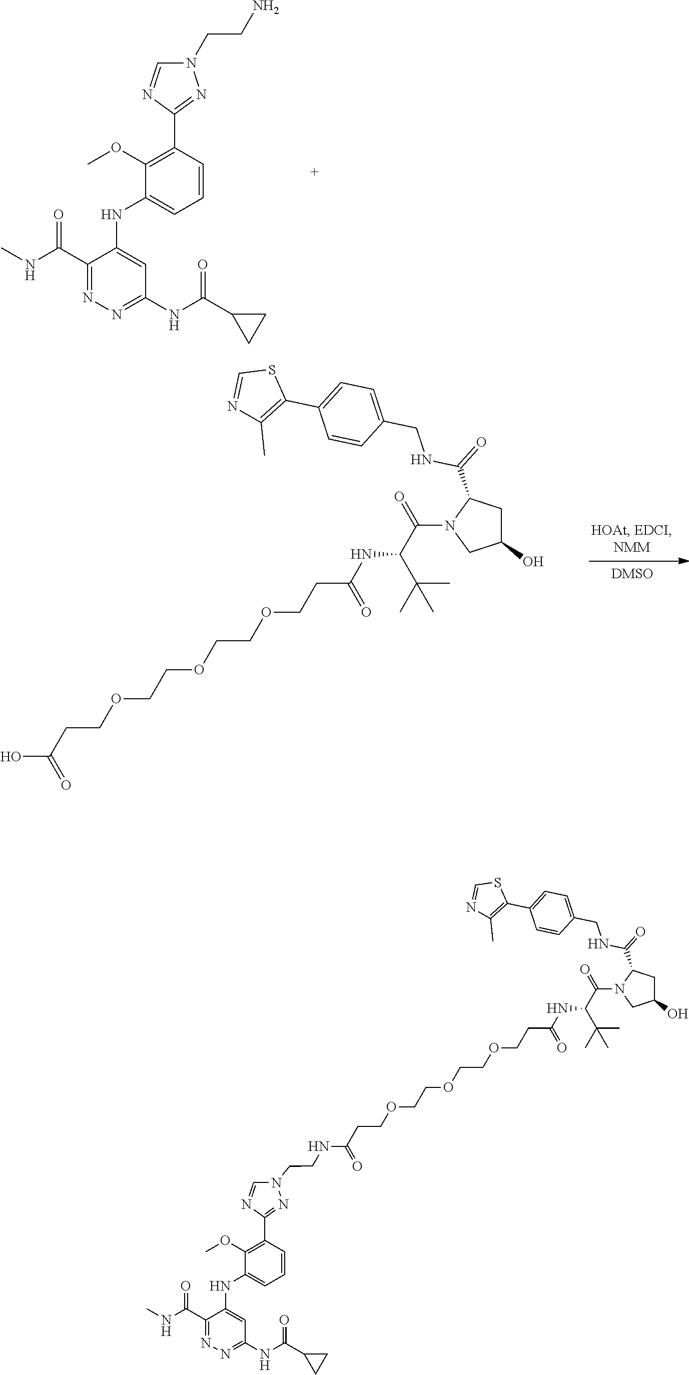

CPD-071 was synthesized following the same procedure for preparing CPD-042 (3.04 mg, yield: 25.1%₀). MS (ESI) m/z=1096.8 [M+H]⁺.

### Example 115: N¹-(2-(3-(3-((6-(Cyclopropanecarboxamido)-3-(methylcarbamoyl)pyridazin-4-yl)amino)-2-methoxyphenyl)-1H-1,2,4-triazol-1-yl)ethyl)-N¹⁶-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-4,7,10,13-tetraoxahexadecanediamide (CPD-072)

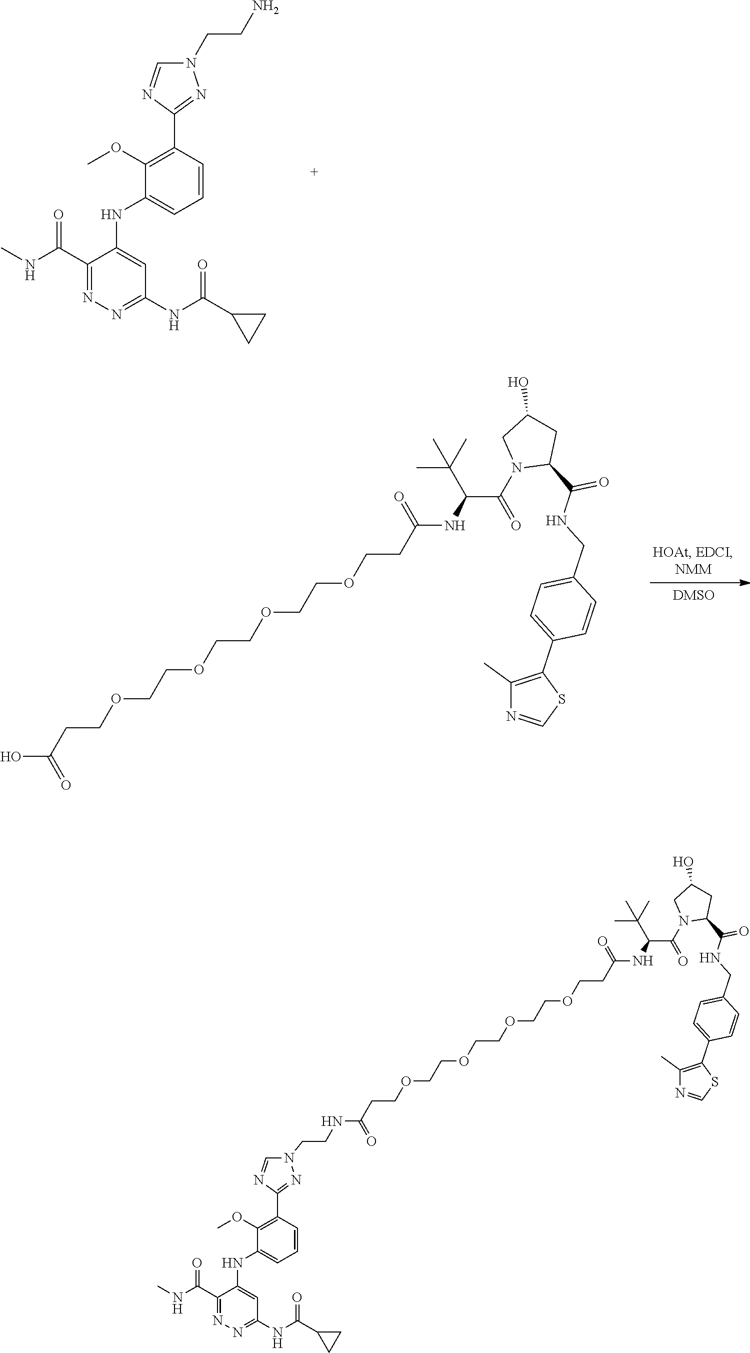 [SMILES: CNC(=O)c1nnc(NC(=O)C2CC2)cc1Nc1cccc(-c2ncn(CCN)n2)c1OC ; Cc1ncsc1-c1ccc(CNC(=O)[C@@H]2C[C@@H](O)CN2C(=O)[C@@H](NC(=O)CCOCCOCCOCCOCCC(=O)O)C(C)(C)C)cc1 ; CNC(=O)c1nnc(NC(=O)C2CC2)cc1Nc1cccc(-c2ncn(CCNC(=O)CCOCCOCCOCCOCCC(=O)N[C@H](C(=O)N3C[C@H](O)C[C@H]3C(=O)NCc3ccc(-c4scnc4C)cc3)C(C)(C)C)n2)c1OC]

CPD-072 was synthesized following the same procedure for preparing CPD-042 (2.62 mg, yield: 20.7%). MS (ESI) m/z=1140.8 [M+H]⁺.

### Example 116: N¹-(2-(3-(3-((6-(Cyclopropanecarboxamido)-3-(methylcarbamoyl)pyridazin-4-yl)amino)-2-methoxyphenyl)-1H-1,2,4-triazol-1-yl)ethyl)-N¹⁷-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-3,6,9,12,15-pentaoxaheptadecanediamide (CPD-073)

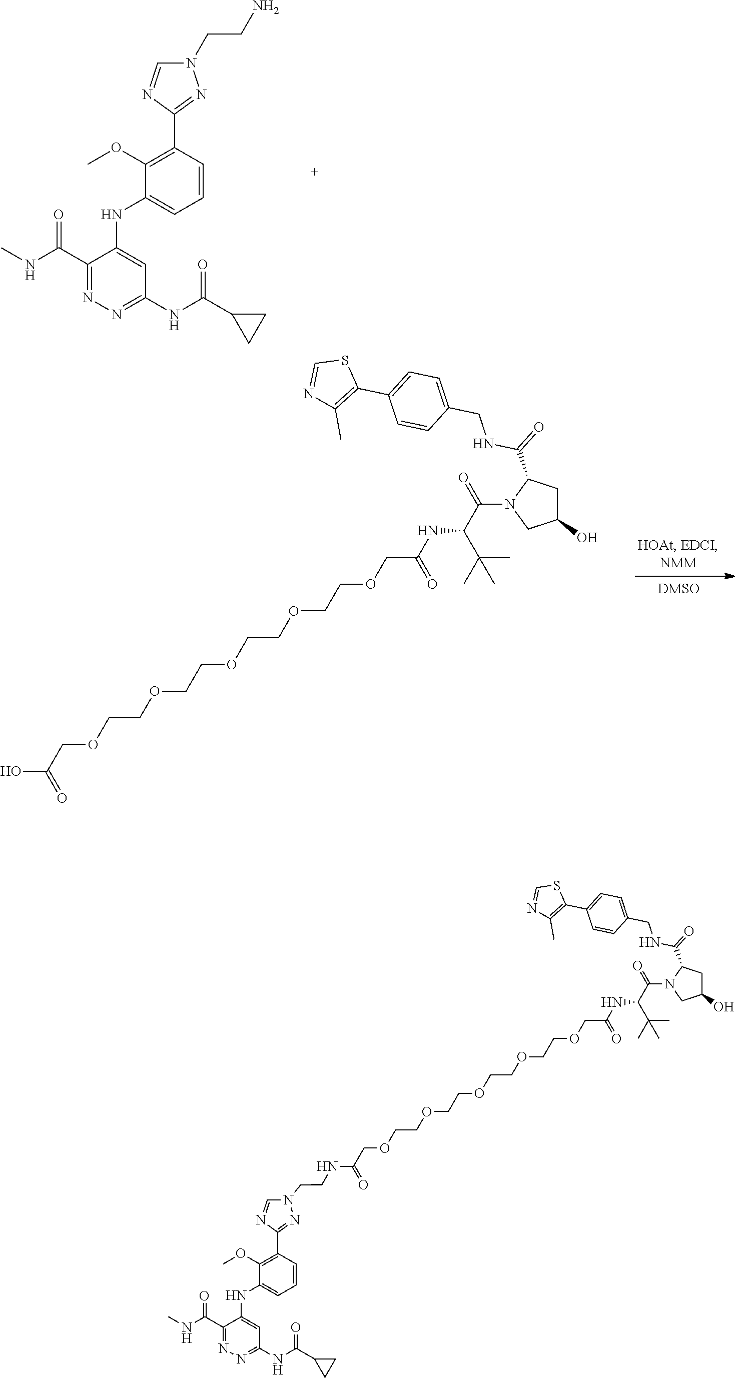 [SMILES: CNC(=O)c1nnc(NC(=O)C2CC2)cc1Nc1cccc(-c2ncn(CCN)n2)c1OC ; Cc1ncsc1-c1ccc(CNC(=O)[C@@H]2C[C@@H](O)CN2C(=O)[C@@H](NC(=O)COCCOCCOCCOCCOCC(=O)O)C(C)(C)C)cc1 ; CNC(=O)c1nnc(NC(=O)C2CC2)cc1Nc1cccc(-c2ncn(CCNC(=O)COCCOCCOCCOCCOCC(=O)N[C@H](C(=O)N3C[C@H](O)C[C@H]3C(=O)NCc3ccc(-c4scnc4C)cc3)C(C)(C)C)n2)c1OC]

CPD-073 was synthesized following the same procedure for preparing CPD-042 (2.48 mg, yield: 19.4%). MS (ESI) m/z=1156.8 [M+H]⁺.

### Example 117: N¹-(2-(3-(3-((6-(Cyclopropanecarboxamido)-3-(methylcarbamoyl)pyridazin-4-yl)amino)-2-methoxyphenyl)-1H-1,2,4-triazol-1-yl)ethyl)-N¹⁹-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-4,7,10,13,16-pentaoxanonadecanediamide (CPD-074)

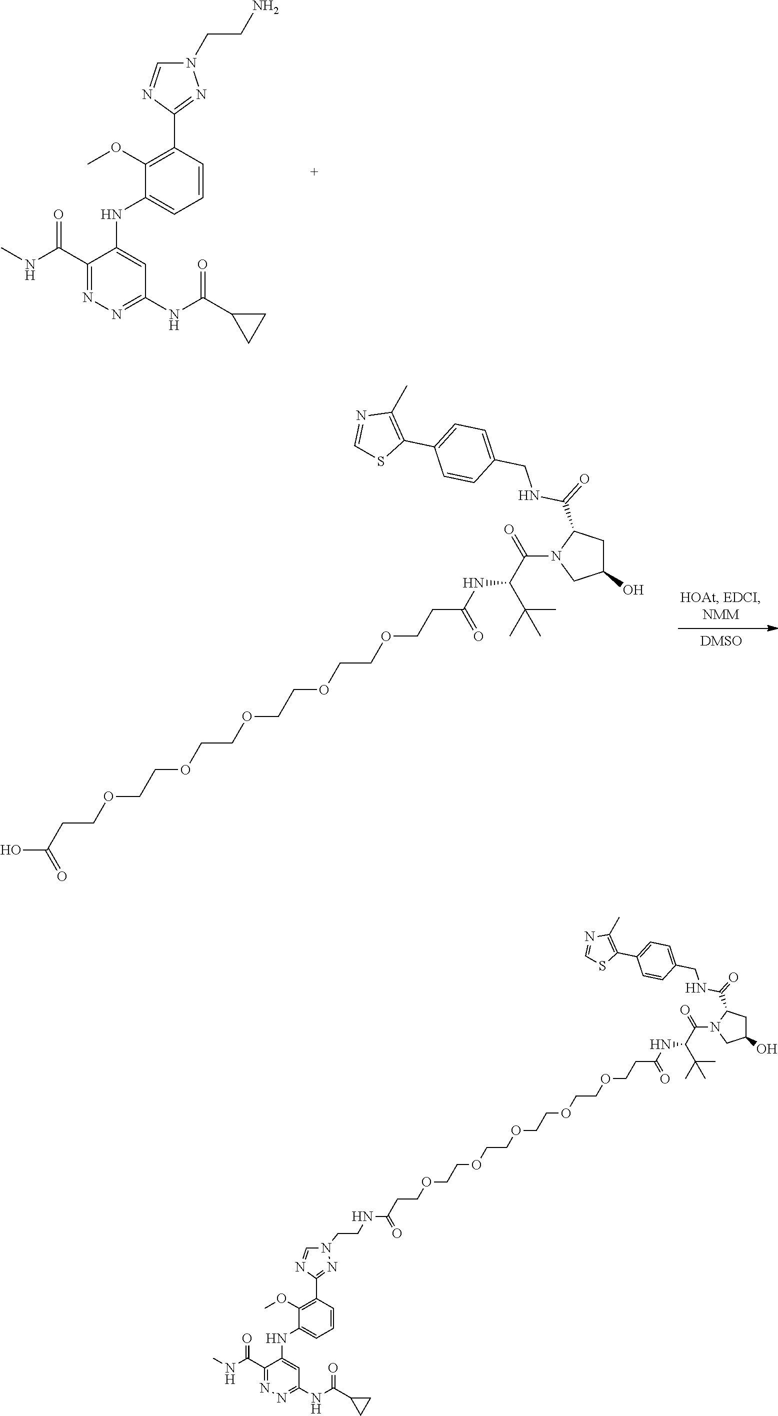 [SMILES: CNC(=O)c1nnc(NC(=O)C2CC2)cc1Nc1cccc(-c2ncn(CCN)n2)c1OC ; Cc1ncsc1-c1ccc(CNC(=O)[C@@H]2C[C@@H](O)CN2C(=O)[C@@H](NC(=O)CCOCCOCCOCCOCCOCCC(=O)O)C(C)(C)C)cc1 ; CNC(=O)c1nnc(NC(=O)C2CC2)cc1Nc1cccc(-c2ncn(CCNC(=O)CCOCCOCCOCCOCCOCCC(=O)N[C@H](C(=O)N3C[C@H](O)C[C@H]3C(=O)NCc3ccc(-c4scnc4C)cc3)C(C)(C)C)n2)c1OC]

CPD-074 was synthesized following the same procedure for preparing CPD-042 (2.92 mg, yield: 22.3%). MS (ESI) m/z=1184.9 [M+H]⁺.

### Example 118: N¹-(2-(3-(3-((6-(Cyclopropanecarboxamido)-3-(methylcarbamoyl)pyridazin-4-yl)amino)-2-methoxyphenyl)-1H-1,2,4-triazol-1-yl)ethyl)-N⁴-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)succinamide (CPD-075)

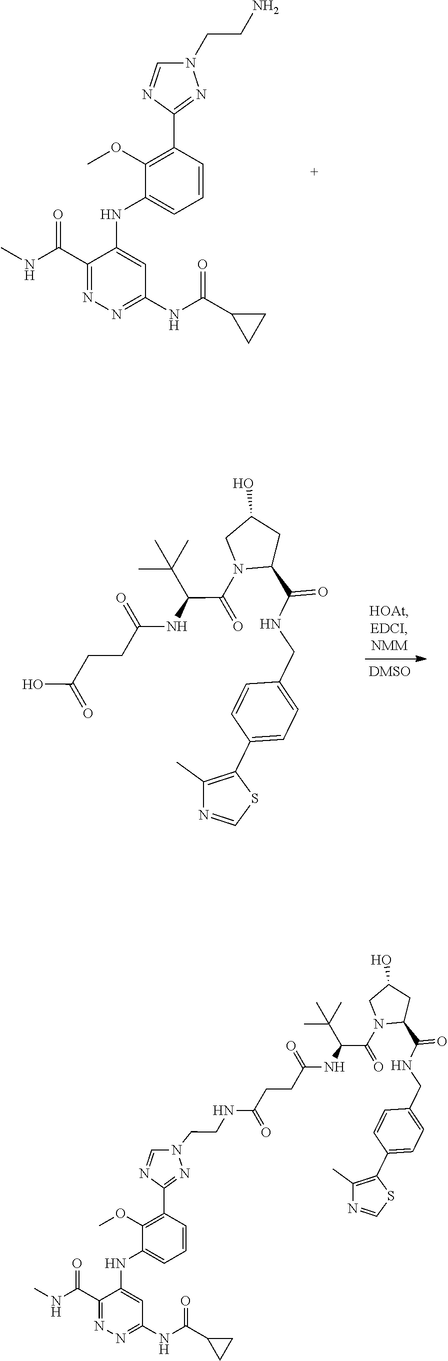 [SMILES: CNC(=O)c1nnc(NC(=O)C2CC2)cc1Nc1cccc(-c2ncn(CCN)n2)c1OC ; Cc1ncsc1-c1ccc(CNC(=O)[C@@H]2C[C@@H](O)CN2C(=O)[C@@H](NC(=O)CCC(=O)O)C(C)(C)C)cc1 ; CNC(=O)c1nnc(NC(=O)C2CC2)cc1Nc1cccc(-c2ncn(CCNC(=O)CCC(=O)N[C@H](C(=O)N3C[C@H](O)C[C@H]3C(=O)NCc3ccc(-c4scnc4C)cc3)C(C)(C)C)n2)c1OC]

CPD-075 was synthesized following the same procedure for preparing CPD-042 (1.94 mg, yield: 18.2%). MS (ESI) m/z=964.8 [M+H]⁺.

### Example 119: N¹-(2-(3-(3-((6-(Cyclopropanecarboxamido)-3-(methylcarbamoyl)pyridazin-4-yl)amino)-2-methoxyphenyl)-1H-1,2,4-triazol-1-yl)ethyl)-N⁵-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)glutaramide (CPD-076)

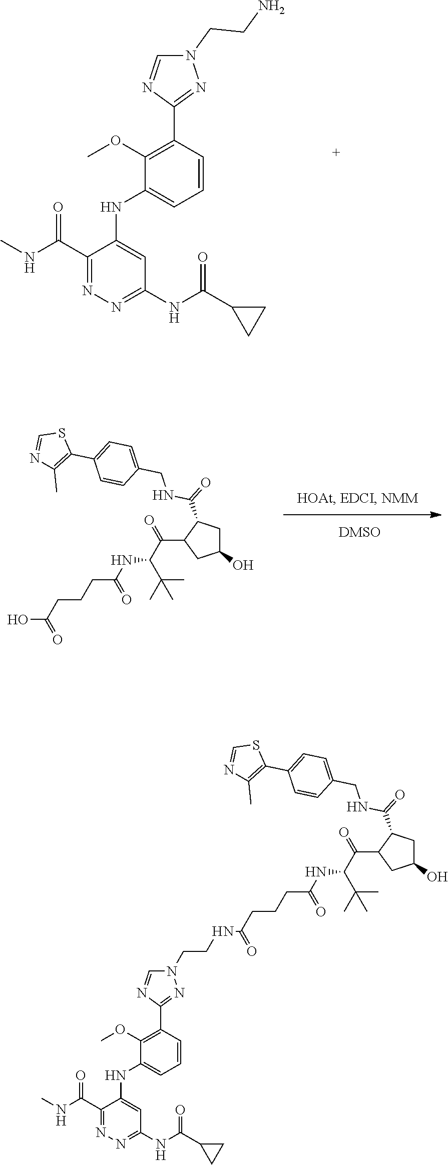 [SMILES: CNC(=O)c1nnc(NC(=O)C2CC2)cc1Nc1cccc(-c2ncn(CCN)n2)c1OC ; Cc1ncsc1-c1ccc(CNC(=O)[C@@H]2C[C@@H](O)CC2C(=O)[C@@H](NC(=O)CCCC(=O)O)C(C)(C)C)cc1 ; CNC(=O)c1nnc(NC(=O)C2CC2)cc1Nc1cccc(-c2ncn(CCNC(=O)CCCC(=O)N[C@H](C(=O)C3C[C@H](O)C[C@H]3C(=O)NCc3ccc(-c4scnc4C)cc3)C(C)(C)C)n2)c1OC]

CPD-076 was synthesized following the same procedure for preparing CPD-042 (2.05 mg, yield: 18.9%). MS (ESI) m/z=978.8 [M+H]⁺.

### Example 120: N¹-(2-(3-(3-((6-(Cyclopropanecarboxamido)-3-(methylcarbamoyl)pyridazin-4-yl)amino)-2-methoxyphenyl)-1H-1,2,4-triazol-1-yl)ethyl)-N⁶-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)adipamide (CPD-077)

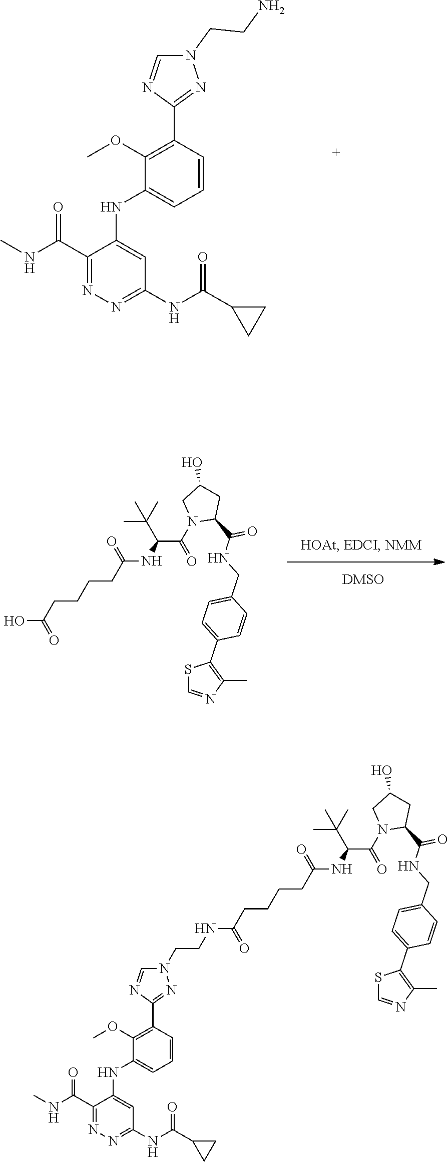 [SMILES: CNC(=O)c1nnc(NC(=O)C2CC2)cc1Nc1cccc(-c2ncn(CCN)n2)c1OC ; Cc1ncsc1-c1ccc(CNC(=O)[C@@H]2C[C@@H](O)CN2C(=O)[C@@H](NC(=O)CCCCC(=O)O)C(C)(C)C)cc1 ; CNC(=O)c1nnc(NC(=O)C2CC2)cc1Nc1cccc(-c2ncn(CCNC(=O)CCCCC(=O)N[C@H](C(=O)N3C[C@H](O)C[C@H]3C(=O)NCc3ccc(-c4scnc4C)cc3)C(C)(C)C)n2)c1OC]

CPD-077 was synthesized following the same procedure for preparing CPD-042 (2.09 mg, yield: 19%). MS (ESI) m/z=992.8 [M+H]⁺.

### Example 121: N¹-(2-(3-(3-((6-(Cyclopropanecarboxamido)-3-(methylcarbamoyl)pyridazin-4-yl)amino)-2-methoxyphenyl)-1H-1,2,4-triazol-1-yl)ethyl)-N⁷-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)heptanediamide (CPD-078)

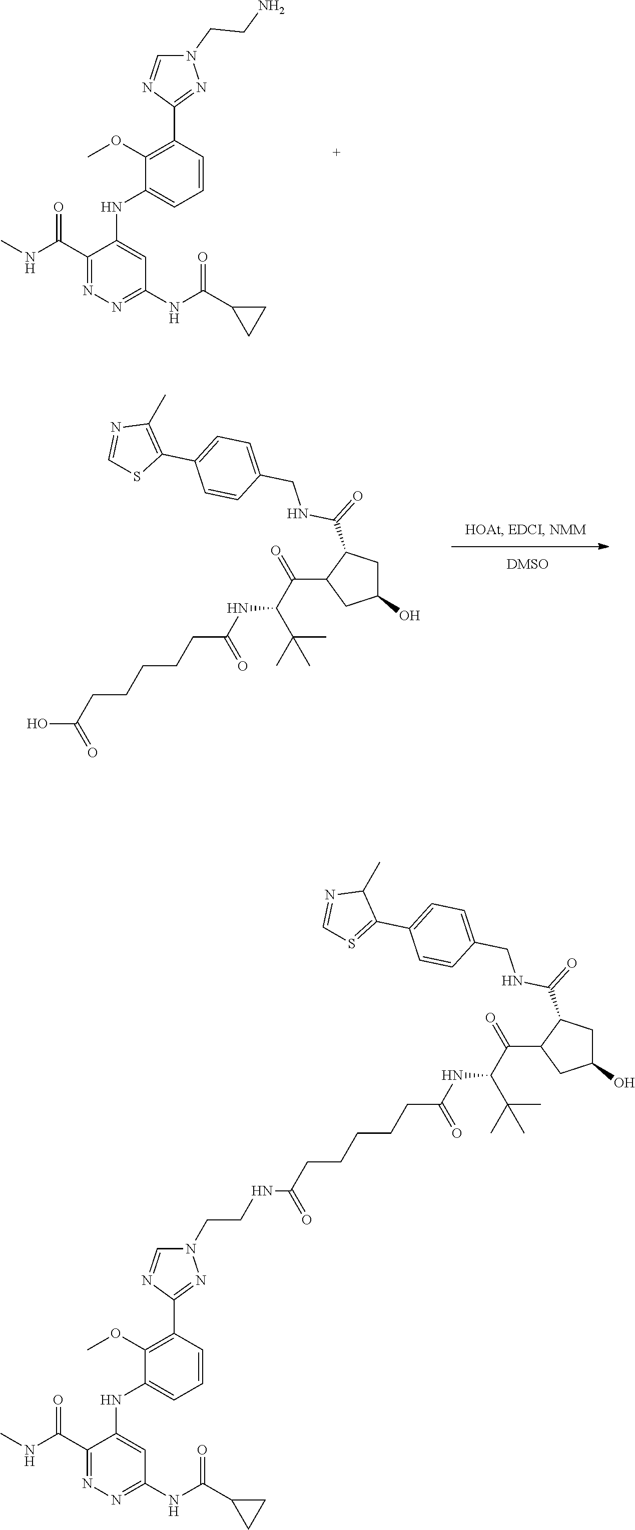

CPD-078 was synthesized following the same procedure for preparing CPD-042 (2.30 mg, yield: 20.6%). MS (ESI) m/z=1006.7 [M+H]

### Example 122: N¹-(2-(3-(3-((6-(Cyclopropanecarboxamido)-3-(methylcarbamoyl)pyridazin-4-yl)amino)-2-methoxyphenyl)-1H-1,2,4-triazol-1-yl)ethyl)-N⁸-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)octanediamide (CPD-079)

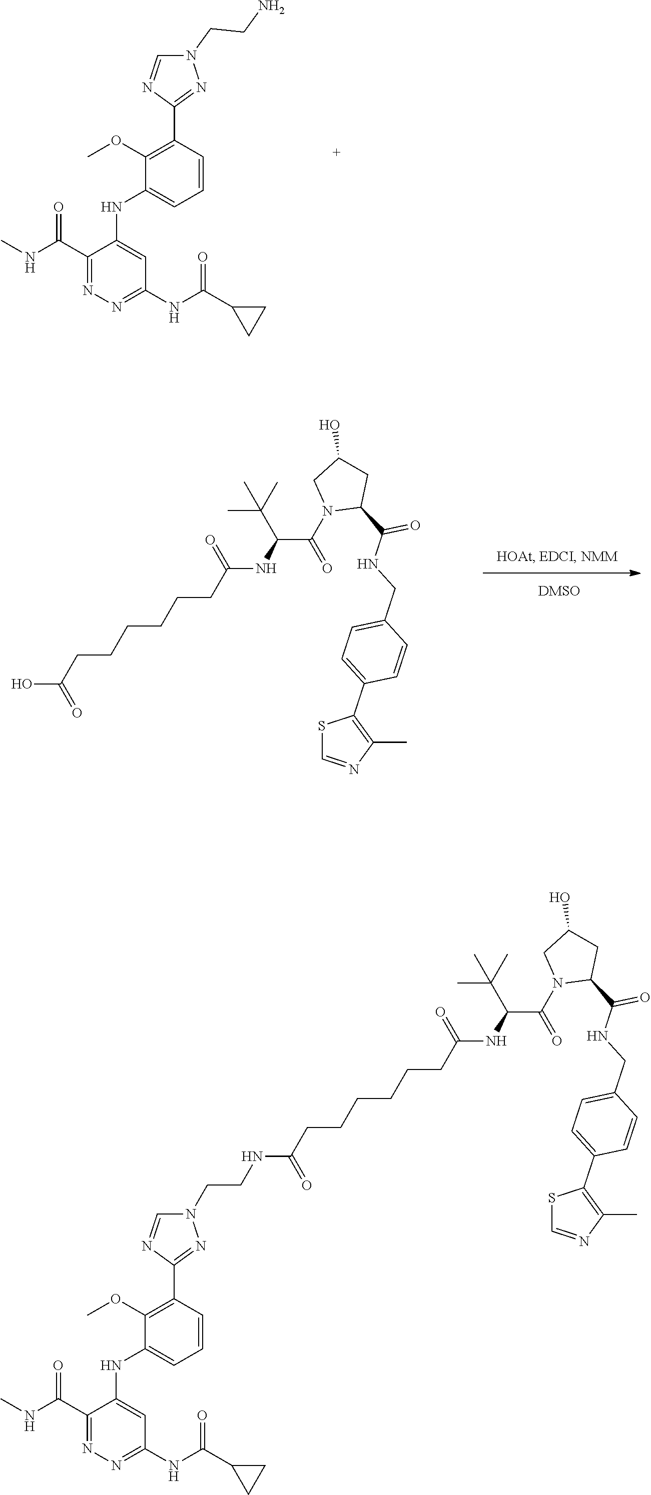 [SMILES: CNC(=O)c1nnc(NC(=O)C2CC2)cc1Nc1cccc(-c2ncn(CCN)n2)c1OC ; Cc1ncsc1-c1ccc(CNC(=O)[C@@H]2C[C@@H](O)CN2C(=O)[C@@H](NC(=O)CCCCCCC(=O)O)C(C)(C)C)cc1 ; CNC(=O)c1nnc(NC(=O)C2CC2)cc1Nc1cccc(-c2ncn(CCNC(=O)CCCCCCC(=O)N[C@H](C(=O)N3C[C@H](O)C[C@H]3C(=O)NCc3ccc(-c4scnc4C)cc3)C(C)(C)C)n2)c1OC]

CPD-079 was synthesized following the same procedure for preparing CPD-042 (2.20 mg, yield: 19.5%). MS (ESI) m/z=1020.8 [M+H]⁺.

### Example 123: N¹-(2-(3-(3-((6-(Cyclopropanecarboxamido)-3-(methylcarbamoyl)pyridazin-4-yl)amino)-2-methoxyphenyl)-1H-1,2,4-triazol-1-yl)ethyl)-N⁹-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)nonanediamide (CPD-080)

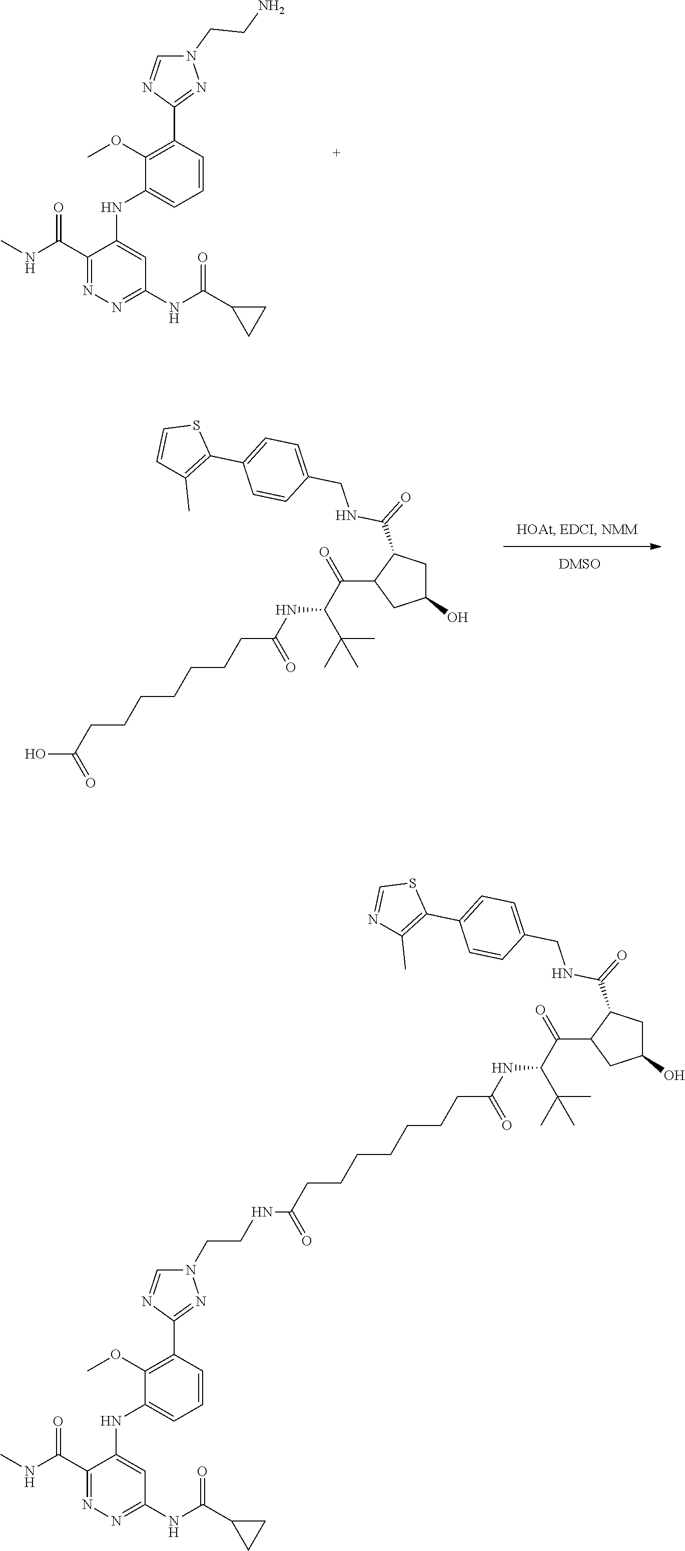 [SMILES: CNC(=O)c1nnc(NC(=O)C2CC2)cc1Nc1cccc(-c2ncn(CCN)n2)c1OC ; Cc1ccsc1-c1ccc(CNC(=O)[C@@H]2C[C@@H](O)CC2C(=O)[C@@H](NC(=O)CCCCCCCC(=O)O)C(C)(C)C)cc1 ; CNC(=O)c1nnc(NC(=O)C2CC2)cc1Nc1cccc(-c2ncn(CCNC(=O)CCCCCCCC(=O)N[C@H](C(=O)C3C[C@H](O)C[C@H]3C(=O)NCc3ccc(-c4scnc4C)cc3)C(C)(C)C)n2)c1OC]

CPD-080 was synthesized following the same procedure for preparing CPD-042 (3.92 mg, yield: 34.2%). MS (ESI) m/z=1034.8 [M+H]⁺.

### Example 124: N¹-(2-(3-(3-((6-(Cyclopropanecarboxamido)-3-(methylcarbamoyl)pyridazin-4-yl)amino)-2-methoxyphenyl)-1H-1,2,4-triazol-1-yl)ethyl)-N¹⁰-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)decanediamide (CPD-081)

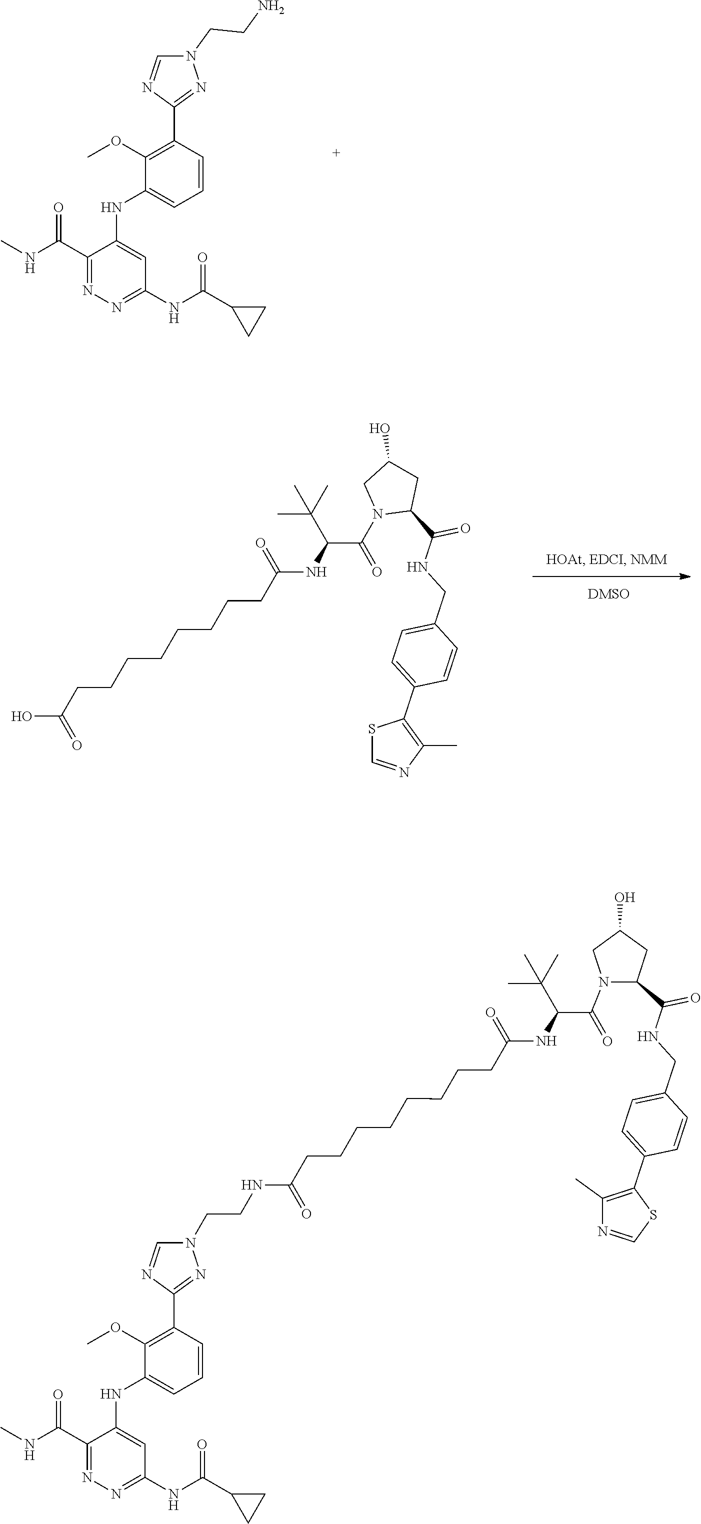 [SMILES: CNC(=O)c1nnc(NC(=O)C2CC2)cc1Nc1cccc(-c2ncn(CCN)n2)c1OC ; Cc1ncsc1-c1ccc(CNC(=O)[C@@H]2C[C@@H](O)CN2C(=O)[C@@H](NC(=O)CCCCCCCCC(=O)O)C(C)(C)C)cc1 ; CNC(=O)c1nnc(NC(=O)C2CC2)cc1Nc1cccc(-c2ncn(CCNC(=O)CCCCCCCCC(=O)N[C@H](C(=O)N3C[C@H](O)C[C@H]3C(=O)NCc3ccc(-c4scnc4C)cc3)C(C)(C)C)n2)c1OC]

CPD-081 was synthesized following the same procedure for preparing CPD-042 (2.31 mg, yield: 19.9%). MS (ESI) m/z=1048.8 [M+H]⁺.

### Example 125: N¹-(2-(3-(3-((6-(Cyclopropanecarboxamido)-3-(methylcarbamoyl)pyridazin-4-yl)amino)-2-methoxyphenyl)-1H-1,2,4-triazol-1-yl)ethyl)-N¹¹-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)undecanediamide (CPD-082)

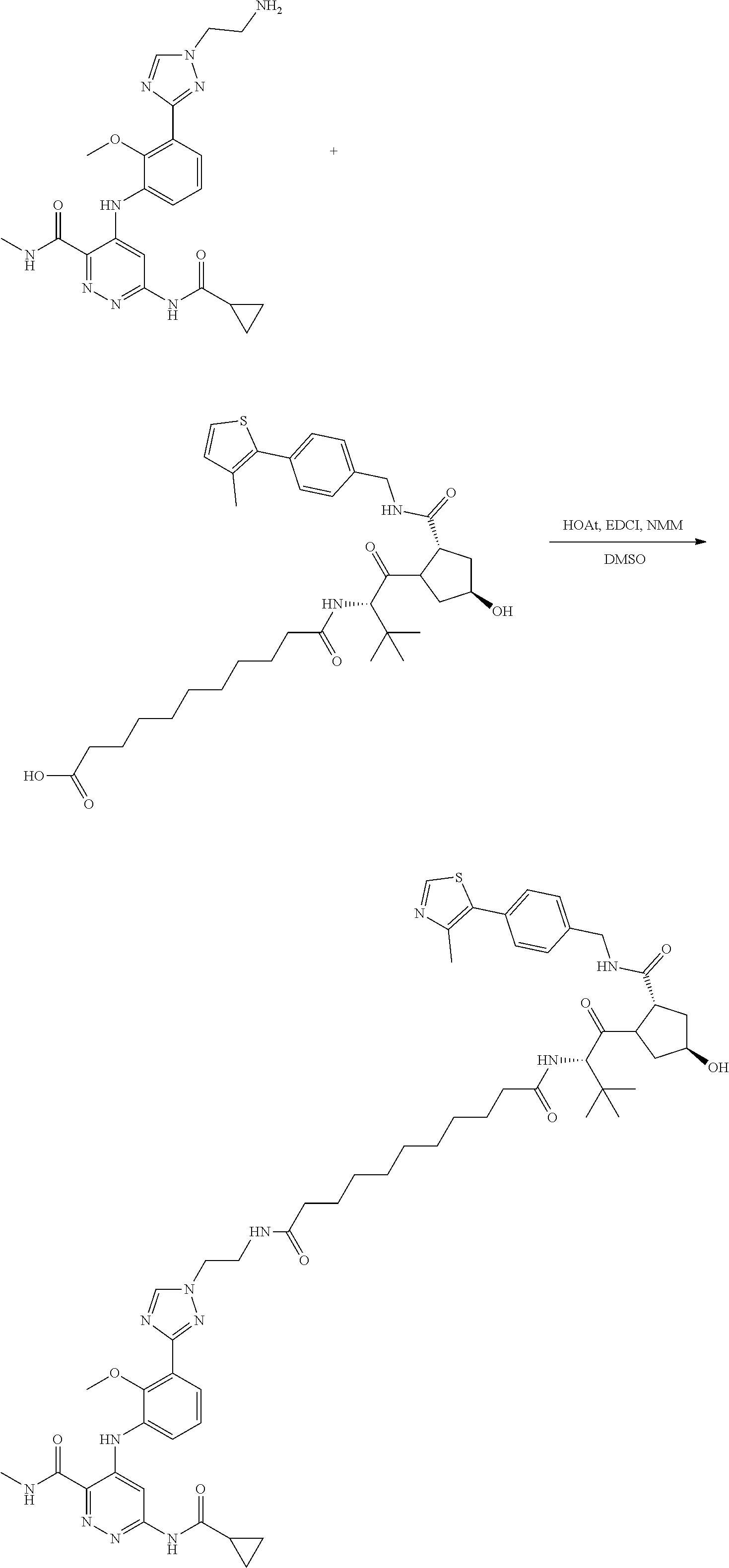 [SMILES: CNC(=O)c1nnc(NC(=O)C2CC2)cc1Nc1cccc(-c2ncn(CCN)n2)c1OC ; Cc1ccsc1-c1ccc(CNC(=O)[C@@H]2C[C@@H](O)CC2C(=O)[C@@H](NC(=O)CCCCCCCCCC(=O)O)C(C)(C)C)cc1 ; CNC(=O)c1nnc(NC(=O)C2CC2)cc1Nc1cccc(-c2ncn(CCNC(=O)CCCCCCCCCC(=O)N[C@H](C(=O)C3C[C@H](O)C[C@H]3C(=O)NCc3ccc(-c4scnc4C)cc3)C(C)(C)C)n2)c1OC]

CPD-082 was synthesized following the same procedure for preparing CPD-042 (2.91 mg, yield: 24.7%₀). MS (ESI) m/z=1062.9 [M+H]⁺.

### Example 126: N¹-((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)-N⁸-(2-((5-((2-methoxy-3-(1-methyl-1H-1,2,4-triazol-3-yl)phenyl)amino)-6-(methylcarbamoyl)pyridazin-3-yl)amino)-2-oxoethyl)octanediamide (CPD-083)

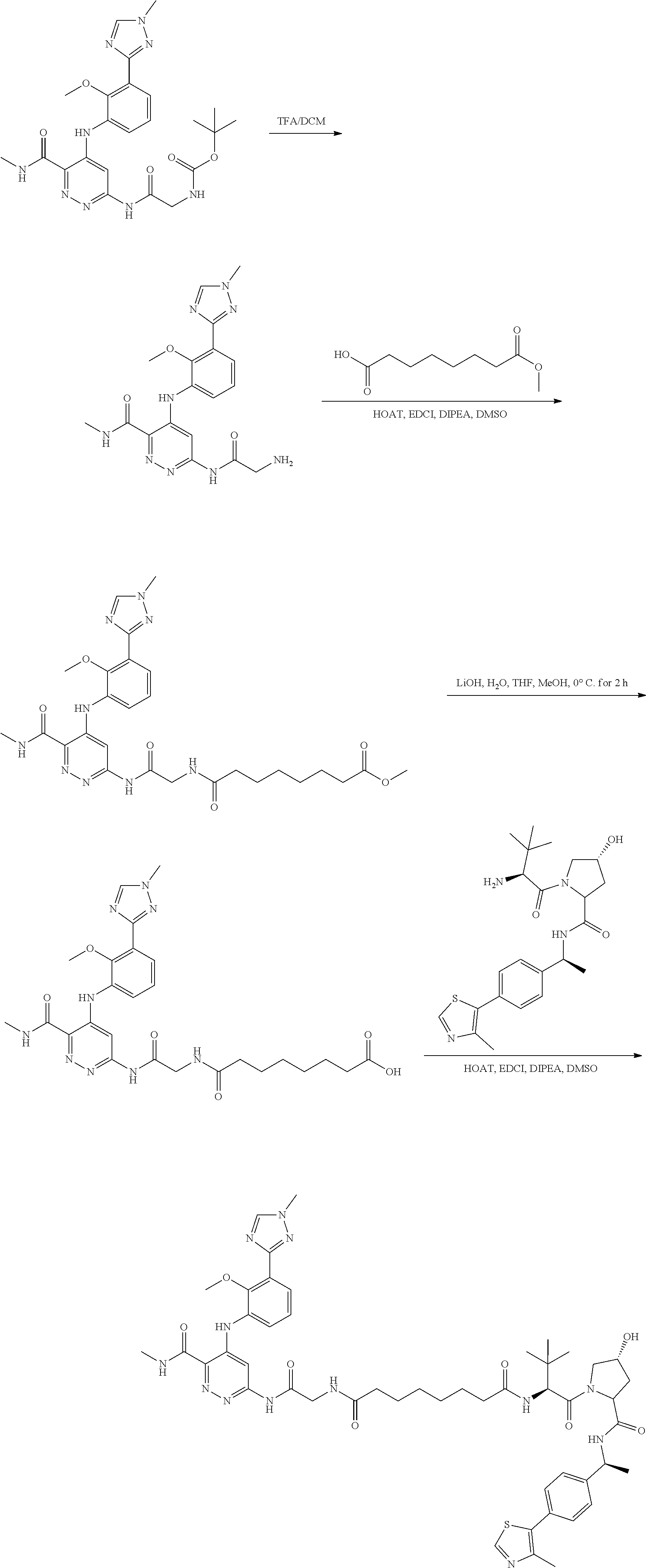

### Step 1. Synthesis of methyl 8-((2-((5-((2-methoxy-3-(1-methyl-1H-1,2,4-triazol-3-yl)phenyl)amino)-6-(methylcarbamoyl)pyridazin-3-yl)amino)-2-oxoethyl)amino)-8-oxooctanoate

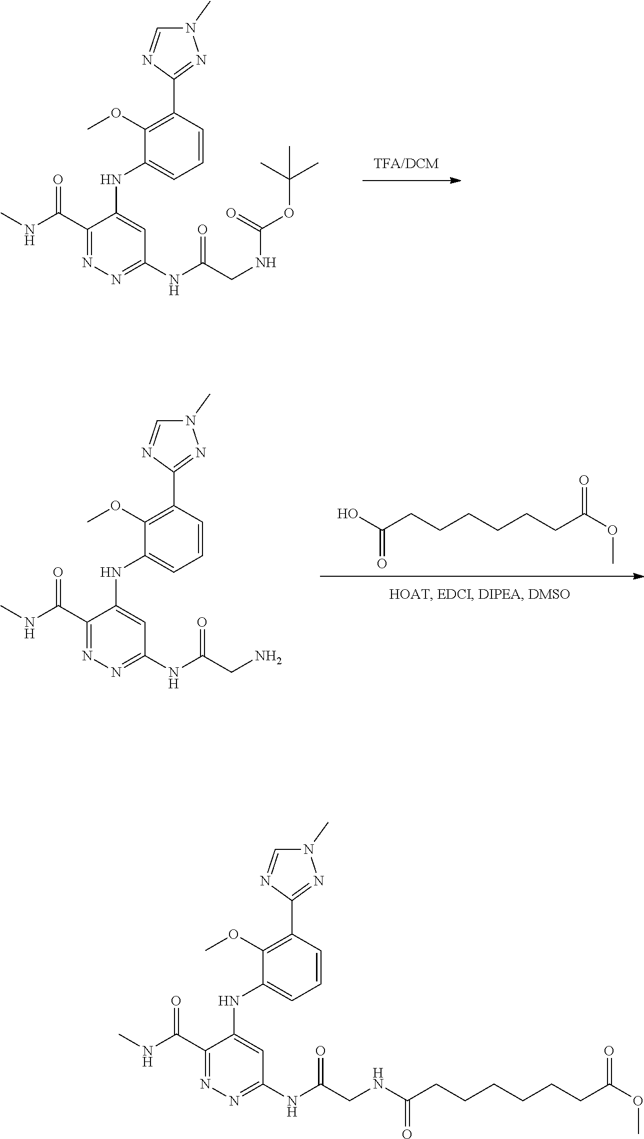

To a solution of tert-butyl (2-454(2-methoxy-3-(1-methyl-1H-1,2,4-triazol-3-yl)phenyl)amino)-6-(methylcarbamoyl)pyridazin-3-yl)amino)-2-oxoethyl)carbamate (150 mg, 293.24 μmol) in DCM (5 mL) was added TFA (1 mL) at rt. After the reaction mixture was stirred at rt for 2 h, it was concentrated to give a crude product (150 mg), which was dissolved in DMSO (5 mL). To the resulting solution were added 8-methoxy-8-oxo-octanoic acid (55.19 mg, 293.24 μmol), HOAt (59.82 mg, 439.85 μmol), EDCI (84.45 mg, 439.85 μmol) and DIPEA (227.39 mg, 1.76 mmol). The resulting mixture was stirred at rt for 16 h, before it was purified by prep-HPLC to give the title compound (140 mg, 82.09% yield). MS (ESI) m/z=582.6 [M+H]⁺.

### Step 2. Synthesis of 8-((2-((5-((2-methoxy-3-(1-methyl-1H-1,2,4-triazol-3-yl)phenyl)amino)-6-(methylcarbamoyl)pyridazin-3-yl)amino)-2-oxoethyl)amino)-8-oxooctanoic acid

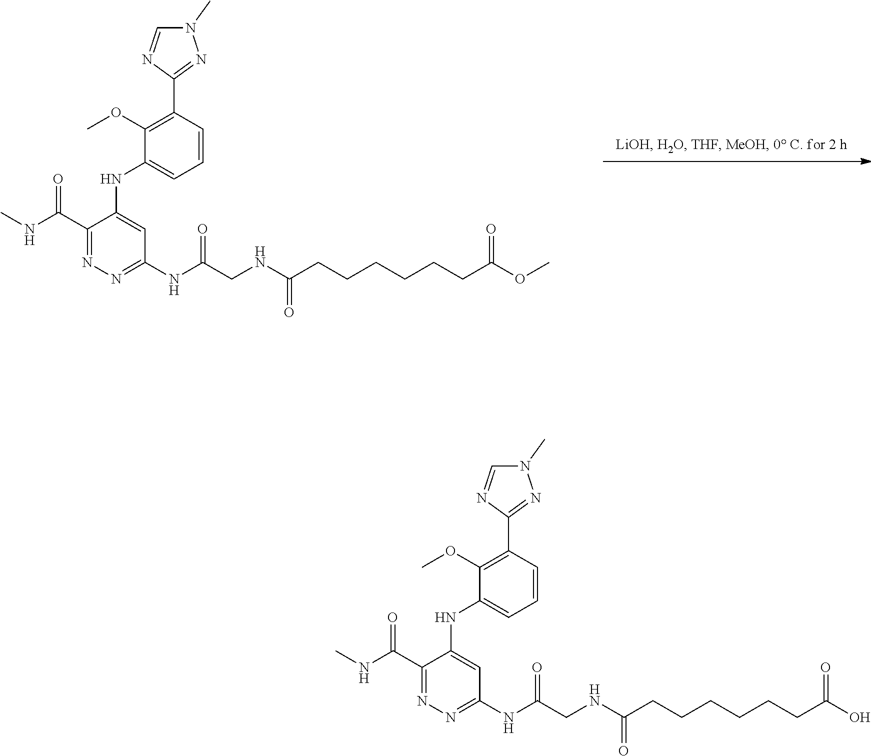

To a solution of methyl 8-((2-((5-((2-methoxy-3-(1-methyl-1H-1,2,4-triazol-3-yl)phenyl)amino)-6-(methylcarbamoyl)pyridazin-3-yl)amino)-2-oxoethyl)amino)-8-oxooctanoate (140 mg, 240.71 μmol) in THF (2 mL), MeOH (2 mL) and water (4 mL) was added LiOH (29 mg, 1.2 mmol) at 0° C. After the reaction mixture was stirred at 0° C. for 2 h, it was purified by prep-HPLC to give the title compound (100 mg, 73.19% yield) as a yellow solid. MS (ESI) m/z=568.5 [M+H]⁺.

### Step 3. Synthesis of N1-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-N8-(2-((5-((2-methoxy-3-(1-methyl-1H-1,2,4-triazol-3-yl)phenyl)amino)-6-(methylcarbamoyl)pyridazin-3-yl)amino)-2-oxoethyl)octanediamide

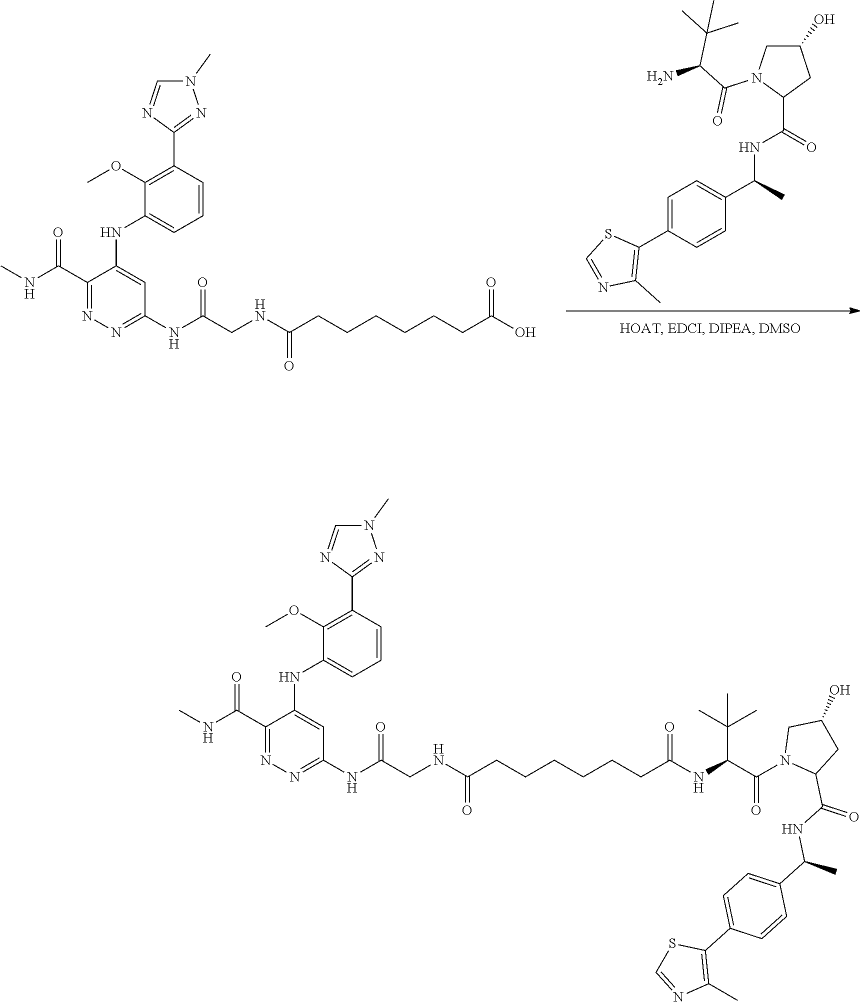 [SMILES: CNC(=O)c1nnc(NC(=O)CNC(=O)CCCCCCC(=O)O)cc1Nc1cccc(-c2ncn(C)n2)c1OC ; Cc1ncsc1-c1ccc([C@H](C)NC(=O)C2C[C@@H](O)CN2C(=O)[C@@H](N)C(C)(C)C)cc1 ; CNC(=O)c1nnc(NC(=O)CNC(=O)CCCCCCC(=O)N[C@H](C(=O)N2C[C@H](O)CC2C(=O)N[C@@H](C)c2ccc(-c3scnc3C)cc2)C(C)(C)C)cc1Nc1cccc(-c2ncn(C)n2)c1OC]

To a solution of 8-((2-((5-((2-methoxy-3-(1-methyl-1H-1,2,4-triazol-3-yl)phenyl)amino)-6-(methylcarbamoyl)pyridazin-3-yl)amino)-2-oxoethyl)amino)-8-oxooctanoic acid (30 mg, 44.01 μmol) and (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (21.17 mg, 44.01 μmol) in DMSO (2.5 mL) were added HOAt (8.98 mg, 66.02 μmol), EDCI (12.68 mg, 66.02 μmol) and DIPEA (34.13 mg, 264.08 μmol) at rt. After the reaction mixture was stirred at rt for 4 h, it was purified by prep-HPLC to give the title compound (6 mg, 13.71% yield) as a white solid. MS (ESI) m/z=995.0 [M+H]⁺.

### Example 127: N¹-((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)-N⁹-(2-((5-((2-methoxy-3-(1-methyl-1H-1,2,4-triazol-3-yl)phenyl)amino)-6-(methylcarbamoyl)pyridazin-3-yl)amino)-2-oxoethyl)nonanediamide (CPD-084)

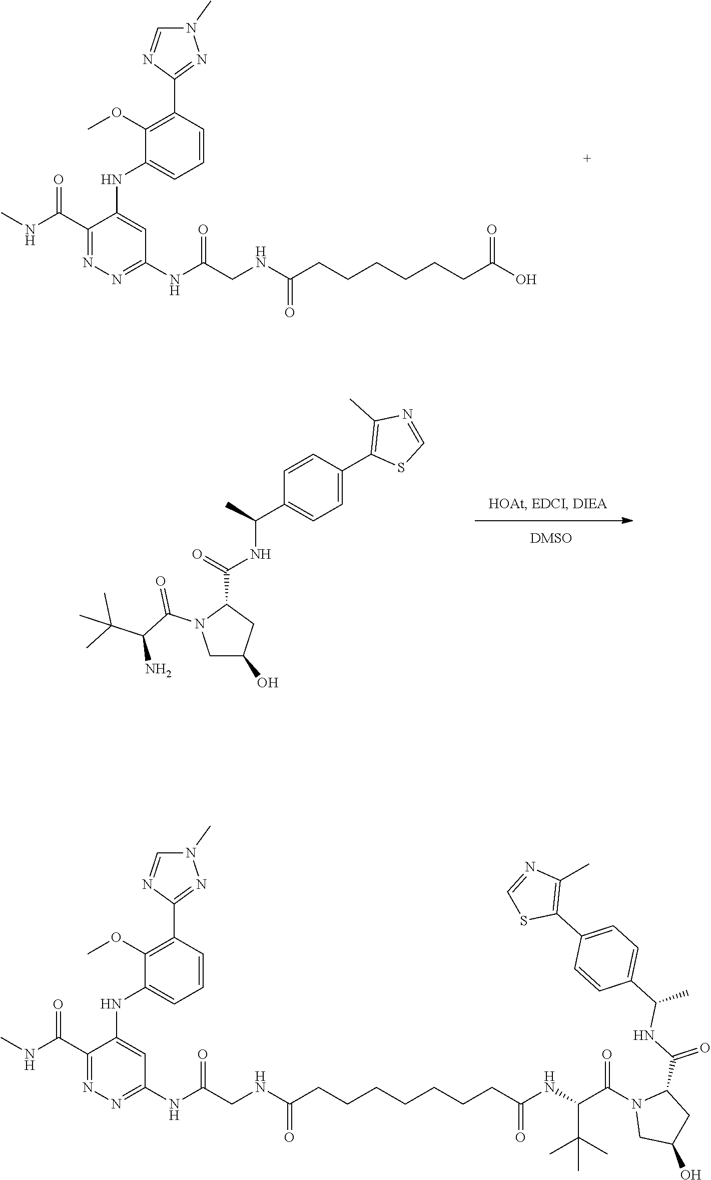 [SMILES: CNC(=O)c1nnc(NC(=O)CNC(=O)CCCCCCC(=O)O)cc1Nc1cccc(-c2ncn(C)n2)c1OC ; Cc1ncsc1-c1ccc([C@H](C)NC(=O)[C@@H]2C[C@@H](O)CN2C(=O)[C@@H](N)C(C)(C)C)cc1 ; CNC(=O)c1nnc(NC(=O)CNC(=O)CCCCCCCC(=O)N[C@H](C(=O)N2C[C@H](O)C[C@H]2C(=O)N[C@@H](C)c2ccc(-c3scnc3C)cc2)C(C)(C)C)cc1Nc1cccc(-c2ncn(C)n2)c1OC]

CPD-084 was synthesized following the same procedure for preparing CPD-083 (1.2 mg, yield: 3.9%). MS (ESI) m/z=1008.8 [M+H]⁺.

### Example 128: N¹-((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)-N⁹-(2-((5-((2-methoxy-3-(1-methyl-1H-1,2,4-triazol-3-yl)phenyl)amino)-6-(methylcarbamoyl)pyridazin-3-yl)amino)-2-oxoethyl)nonanediamide (CPD-085)

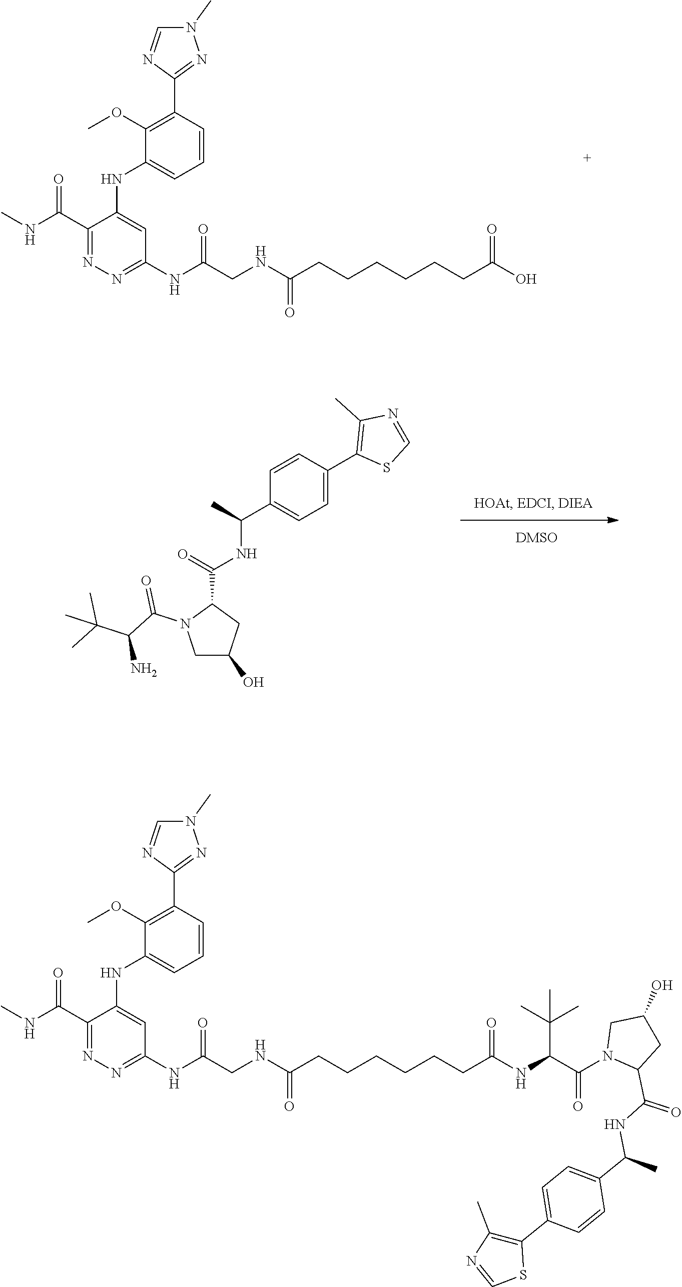 [SMILES: CNC(=O)c1nnc(NC(=O)CNC(=O)CCCCCCC(=O)O)cc1Nc1cccc(-c2ncn(C)n2)c1OC ; Cc1ncsc1-c1ccc([C@H](C)NC(=O)[C@@H]2C[C@@H](O)CN2C(=O)[C@@H](N)C(C)(C)C)cc1 ; CNC(=O)c1nnc(NC(=O)CNC(=O)CCCCCCC(=O)N[C@H](C(=O)N2C[C@H](O)CC2C(=O)N[C@@H](C)c2ccc(-c3scnc3C)cc2)C(C)(C)C)cc1Nc1cccc(-c2ncn(C)n2)c1OC]

CPD-085 was synthesized following the same procedure for preparing CPD-083 (6 mg, yield: 8.0%). MS (ESI) m/z=1022.8 [M+H]⁺.

### Example 129: 6-(2-(2-((S)-3-((2S,4R)-1-((S)-2-(1-Fluorocyclopropane-1-carboxamido)-3,3-dimethylbutanoyl)-4-hydroxypyrrolidine-2-carboxamido)-3-(4-(4-methylthiazol-5-yl)phenyl)propanamido)acetamido)acetamido)-4-((2-methoxy-3-(1-methyl-1H-1,2,4-triazol-3-yl)phenyl)amino)-N-methylpyridazine-3-carboxamide (CPD-086)

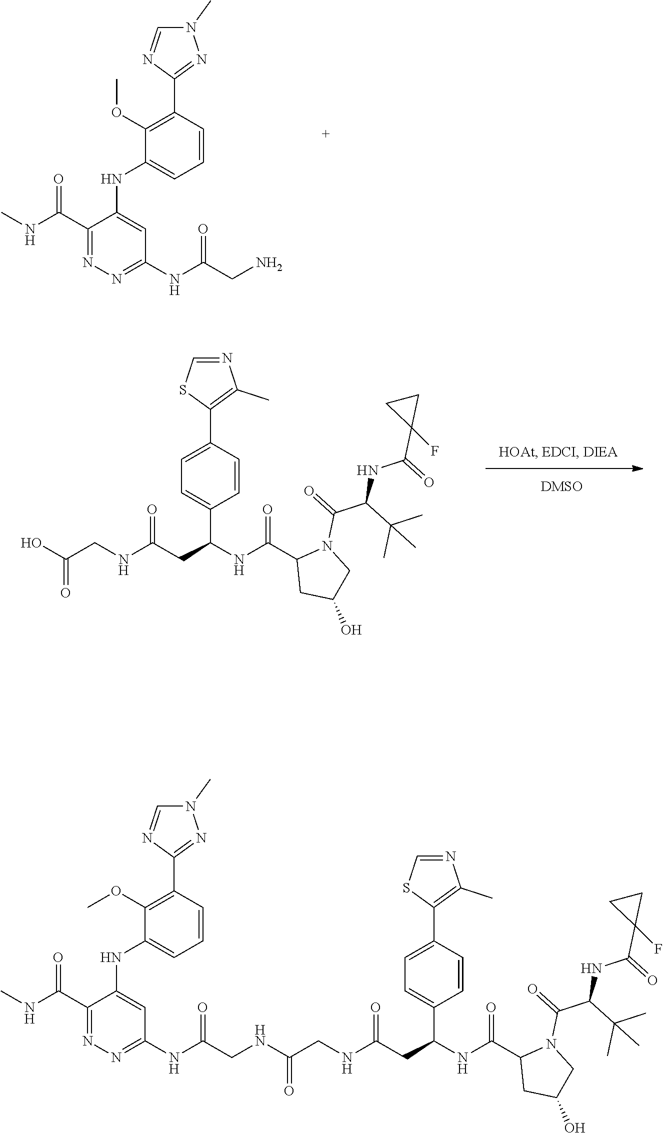 [SMILES: CNC(=O)c1nnc(NC(=O)CN)cc1Nc1cccc(-c2ncn(C)n2)c1OC ; Cc1ncsc1-c1ccc([C@H](CC(=O)NCC(=O)O)NC(=O)C2C[C@@H](O)CN2C(=O)[C@@H](NC(=O)C2(F)CC2)C(C)(C)C)cc1 ; CNC(=O)c1nnc(NC(=O)CNC(=O)CNC(=O)C[C@H](NC(=O)C2C[C@@H](O)CN2C(=O)[C@@H](NC(=O)C2(F)CC2)C(C)(C)C)c2ccc(-c3scnc3C)cc2)cc1Nc1cccc(-c2ncn(C)n2)c1OC]

CPD-086 was synthesized following the same procedure for preparing CPD-042 (1.44 mg, yield: 11.6%). MS (ESI) m/z=1026.0 [M+H]⁺.

### Example 130: 6-(2-(3-((S)-3-((2S,4R)-1-((S)-2-(1-Fluorocyclopropane-1-carboxamido)-3,3-dimethylbutanoyl)-4-hydroxypyrrolidine-2-carboxamido)-3-(4-(4-methylthiazol-5-yl)phenyl)propanamido)propanamido)acetamido)-4-((2-methoxy-3-(1-methyl-1H-1,2,4-triazol-3-yl)phenyl)amino)-N-methylpyridazine-3-carboxamide (CPD-087)

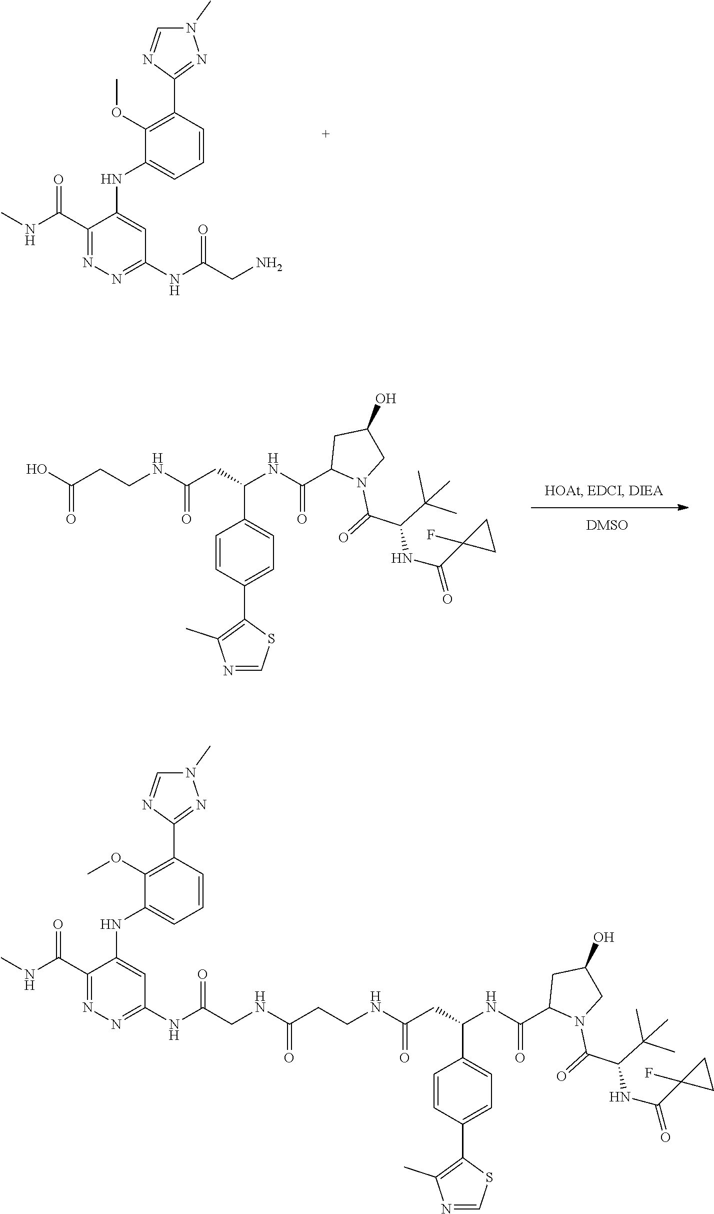 [SMILES: CNC(=O)c1nnc(NC(=O)CN)cc1Nc1cccc(-c2ncn(C)n2)c1OC ; Cc1ncsc1-c1ccc([C@H](CC(=O)NCCC(=O)O)NC(=O)C2C[C@@H](O)CN2C(=O)[C@@H](NC(=O)C2(F)CC2)C(C)(C)C)cc1 ; CNC(=O)c1nnc(NC(=O)CNC(=O)CCNC(=O)C[C@H](NC(=O)C2C[C@@H](O)CN2C(=O)[C@@H](NC(=O)C2(F)CC2)C(C)(C)C)c2ccc(-c3scnc3C)cc2)cc1Nc1cccc(-c2ncn(C)n2)c1OC]

CPD-087 was synthesized following the same procedure for preparing CPD-042 (1.33 mg, yield: 10.6%). MS (ESI) m/z=1040.0 [M+H]⁺.

### Example 131: 6-(2-(4-((S)-3-((2S,4R)-1-((S)-2-(1-Fluorocyclopropane-1-carboxamido)-3,3-dimethylbutanoyl)-4-hydroxypyrrolidine-2-carboxamido)-3-(4-(4-methylthiazol-5-yl)phenyl)propanamido)butanamido)acetamido)-4-((2-methoxy-3-(1-methyl-1H-1,2,4-triazol-3-yl)phenyl)amino)-N-methylpyridazine-3-carboxamide (CPD-088)

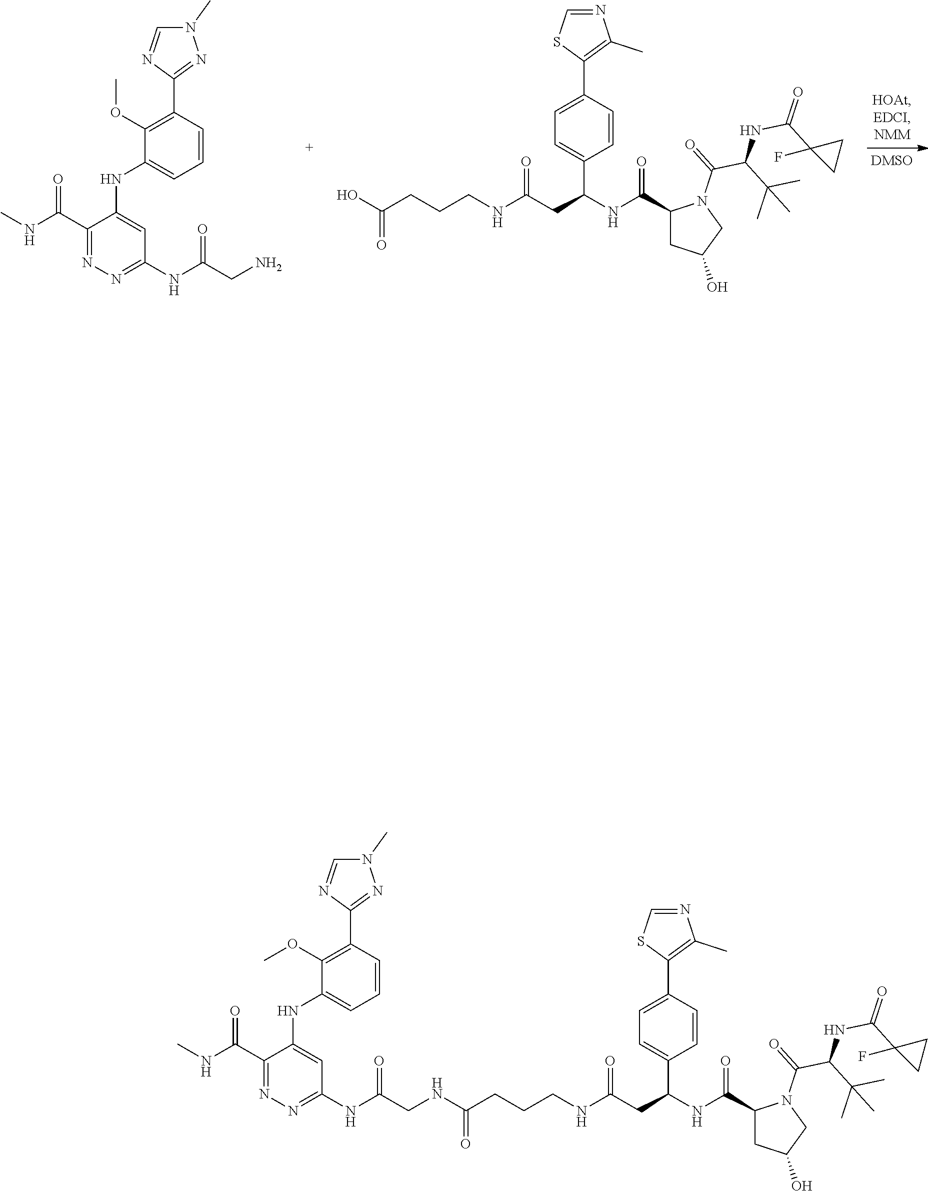 [SMILES: CNC(=O)c1nnc(NC(=O)CN)cc1Nc1cccc(-c2ncn(C)n2)c1OC ; Cc1ncsc1-c1ccc([C@H](CC(=O)NCCCC(=O)O)NC(=O)[C@@H]2C[C@@H](O)CN2C(=O)[C@@H](NC(=O)C2(F)CC2)C(C)(C)C)cc1 ; CNC(=O)c1nnc(NC(=O)CNC(=O)CCCNC(=O)C[C@H](NC(=O)[C@@H]2C[C@@H](O)CN2C(=O)[C@@H](NC(=O)C2(F)CC2)C(C)(C)C)c2ccc(-c3scnc3C)cc2)cc1Nc1cccc(-c2ncn(C)n2)c1OC]

CPD-088 was synthesized following the same procedure for preparing CPD-042 (1.75 mg, yield: 13.8%). MS (ESI) m/z=1054.1 [M+H]⁺.

### Example 132: 6-(2-(5-((S)-3-((2S,4R)-1-((S)-2-(1-Fluorocyclopropane-1-carboxamido)-3,3-dimethylbutanoyl)-4-hydroxypyrrolidine-2-carboxamido)-3-(4-(4-methylthiazol-5-yl)phenyl)propanamido)pentanamido)acetamido)-4-((2-methoxy-3-(1-methyl-1H-1,2,4-triazol-3-yl)phenyl)amino)-N-methylpyridazine-3-carboxamide (CPD-089)

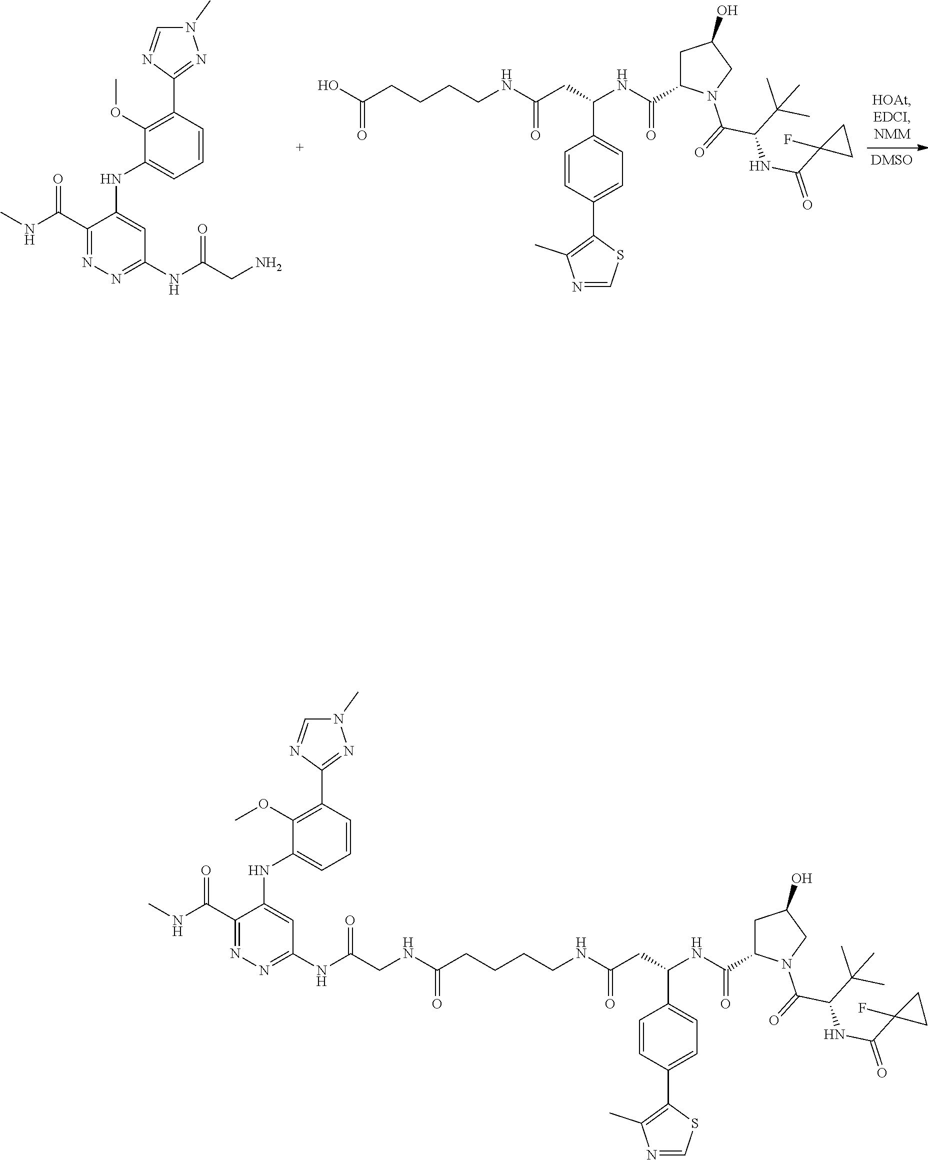 [SMILES: CNC(=O)c1nnc(NC(=O)CN)cc1Nc1cccc(-c2ncn(C)n2)c1OC ; Cc1ncsc1-c1ccc([C@H](CC(=O)NCCCCC(=O)O)NC(=O)[C@@H]2C[C@@H](O)CN2C(=O)[C@@H](NC(=O)C2(F)CC2)C(C)(C)C)cc1 ; CNC(=O)c1nnc(NC(=O)CNC(=O)CCCCNC(=O)C[C@H](NC(=O)[C@@H]2C[C@@H](O)CN2C(=O)[C@@H](NC(=O)C2(F)CC2)C(C)(C)C)c2ccc(-c3scnc3C)cc2)cc1Nc1cccc(-c2ncn(C)n2)c1OC]

CPD-089 was synthesized following the same procedure for preparing CPD-042 (2.55 mg, yield: 19.8%). MS (ESI) m/z=1068.0 [M+H]⁺.

### Example 133: 6-(2-(6-((S)-3-((2S,4R)-1-((S)-2-(1-Fluorocyclopropane-1-carboxamido)-3,3-dimethylbutanoyl)-4-hydroxypyrrolidine-2-carboxamido)-3-(4-(4-methylthiazol-5-yl)phenyl)propanamido)hexanamido)acetamido)-4-((2-methoxy-3-(1-methyl-1H-1,2,4-triazol-3-yl)phenyl)amino)-N-methylpyridazine-3-carboxamide CPD-090)

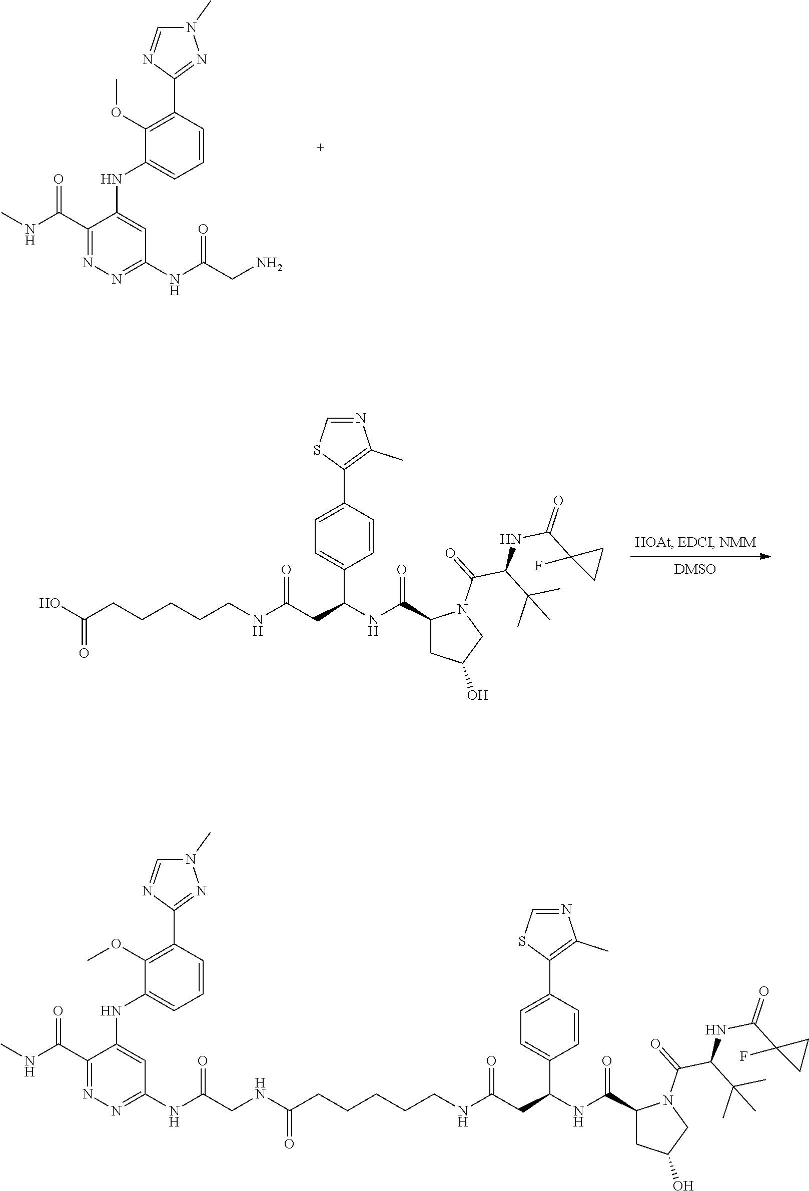 [SMILES: CNC(=O)c1nnc(NC(=O)CN)cc1Nc1cccc(-c2ncn(C)n2)c1OC ; Cc1ncsc1-c1ccc([C@H](CC(=O)NCCCCCC(=O)O)NC(=O)[C@@H]2C[C@@H](O)CN2C(=O)[C@@H](NC(=O)C2(F)CC2)C(C)(C)C)cc1 ; CNC(=O)c1nnc(NC(=O)CNC(=O)CCCCCNC(=O)C[C@H](NC(=O)[C@@H]2C[C@@H](O)CN2C(=O)[C@@H](NC(=O)C2(F)CC2)C(C)(C)C)c2ccc(-c3scnc3C)cc2)cc1Nc1cccc(-c2ncn(C)n2)c1OC]

CPD-090 was synthesized following the same procedure for preparing CPD-042 (2.96 mg, yield: 22.6%). MS (ESI) m/z=1082.1 [M+H]⁺.

### Example 134: 6-(2-(7-((S)-3-((2S,4R)-1-((S)-2-(1-Fluorocyclopropane-1-carboxamido)-3,3-dimethylbutanoyl)-4-hydroxypyrrolidine-2-carboxamido)-3-(4-(4-methylthiazol-5-yl)phenyl)propanamido)heptanamido)acetamido)-4-((2-methoxy-3-(1-methyl-1H-1,2,4-triazol-3-yl)phenyl)amino)-N-methylpyridazine-3-carboxamide (CPD-091)

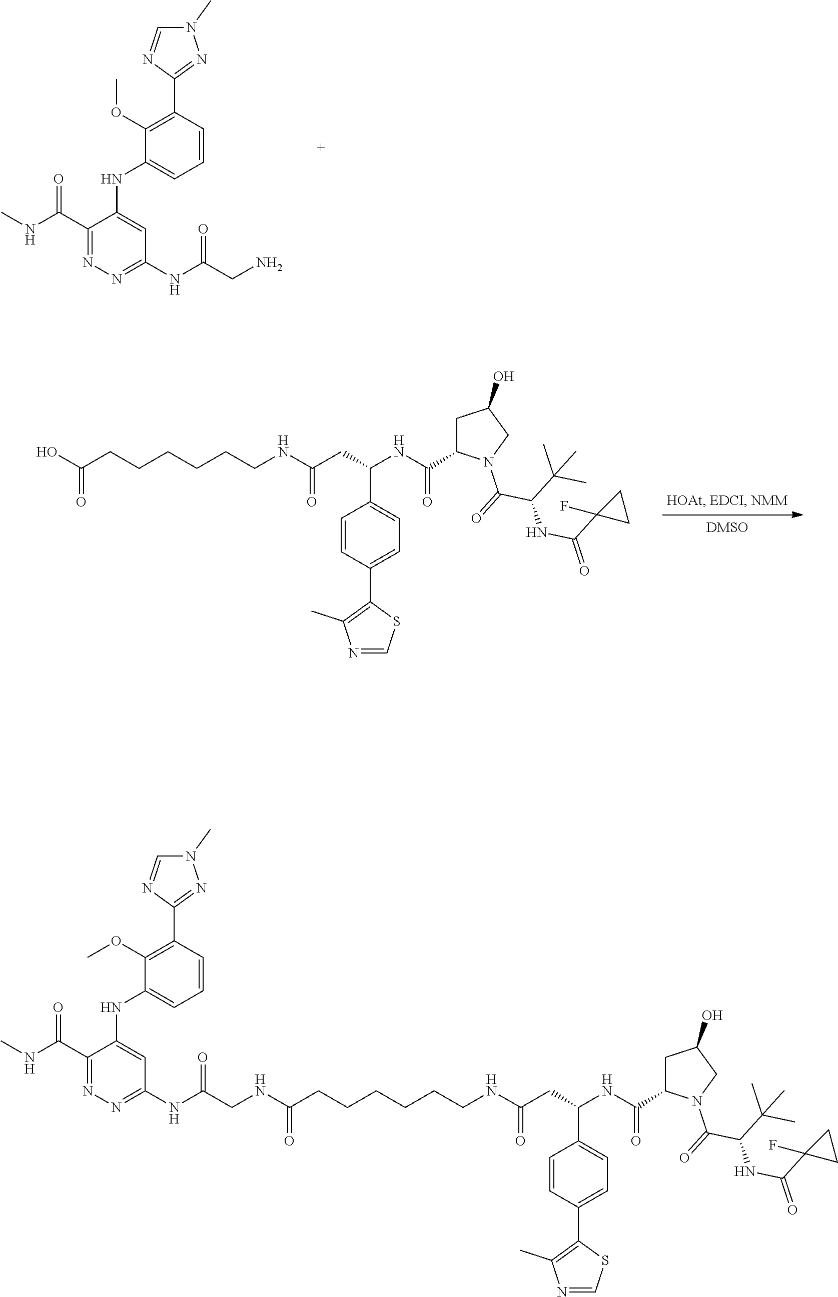 [SMILES: CNC(=O)c1nnc(NC(=O)CN)cc1Nc1cccc(-c2ncn(C)n2)c1OC ; Cc1ncsc1-c1ccc([C@H](CC(=O)NCCCCCCC(=O)O)NC(=O)[C@@H]2C[C@@H](O)CN2C(=O)[C@@H](NC(=O)C2(F)CC2)C(C)(C)C)cc1 ; CNC(=O)c1nnc(NC(=O)CNC(=O)CCCCCCNC(=O)C[C@H](NC(=O)[C@@H]2C[C@@H](O)CN2C(=O)[C@@H](NC(=O)C2(F)CC2)C(C)(C)C)c2ccc(-c3scnc3C)cc2)cc1Nc1cccc(-c2ncn(C)n2)c1OC]

CPD-091 was synthesized following the same procedure for preparing CPD-042 (2.75 mg, yield: 20.7%). MS (ESI) m/z=1096.1 [M+H]⁺.

### Example 135: 6-(2-(8-((S)-3-((2S,4R)-1-((S)-2-(1-Fluorocyclopropane-1-carboxamido)-3,3-dimethylbutanoyl)-4-hydroxypyrrolidine-2-carboxamido)-3-(4-(4-methylthiazol-5-yl)phenyl)propanamido)octanamido)acetamido)-4-((2-methoxy-3-(1-methyl-1H-1,2,4-triazol-3-yl)phenyl)amino)-N-methylpyridazine-3-carboxamide (CPD-092)

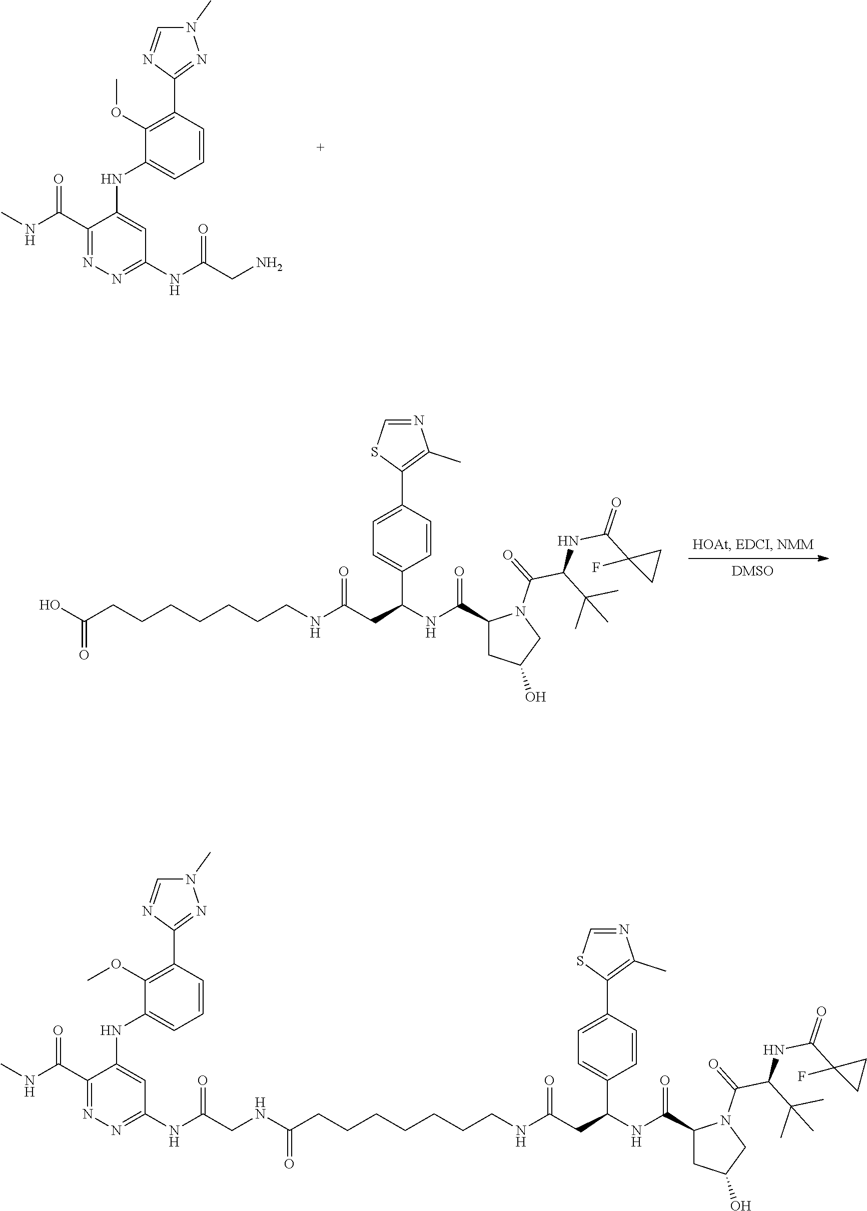 [SMILES: CNC(=O)c1nnc(NC(=O)CN)cc1Nc1cccc(-c2ncn(C)n2)c1OC ; Cc1ncsc1-c1ccc([C@H](CC(=O)NCCCCCCCC(=O)O)NC(=O)[C@@H]2C[C@@H](O)CN2C(=O)[C@@H](NC(=O)C2(F)CC2)C(C)(C)C)cc1 ; CNC(=O)c1nnc(NC(=O)CNC(=O)CCCCCCCNC(=O)C[C@H](NC(=O)[C@@H]2C[C@@H](O)CN2C(=O)[C@@H](NC(=O)C2(F)CC2)C(C)(C)C)c2ccc(-c3scnc3C)cc2)cc1Nc1cccc(-c2ncn(C)n2)c1OC]

CPD-092 was synthesized following the same procedure for preparing CPD-042 (1.86 mg, yield: 13.9%). MS (ESI) m/z=1110.2 [M+H]⁺.

### Example 136: 6-(2-(9-((S)-3-((2S,4R)-1-((S)-2-(1-Fluorocyclopropane-1-carboxamido)-3,3-dimethylbutanoyl)-4-hydroxypyrrolidine-2-carboxamido)-3-(4-(4-methylthiazol-5-yl)phenyl)propanamido)nonanamido)acetamido)-4-((2-methoxy-3-(1-methyl-1H-1,2,4-triazol-3-yl)phenyl)amino)-N-methylpyridazine-3-carboxamide (CPD-093)

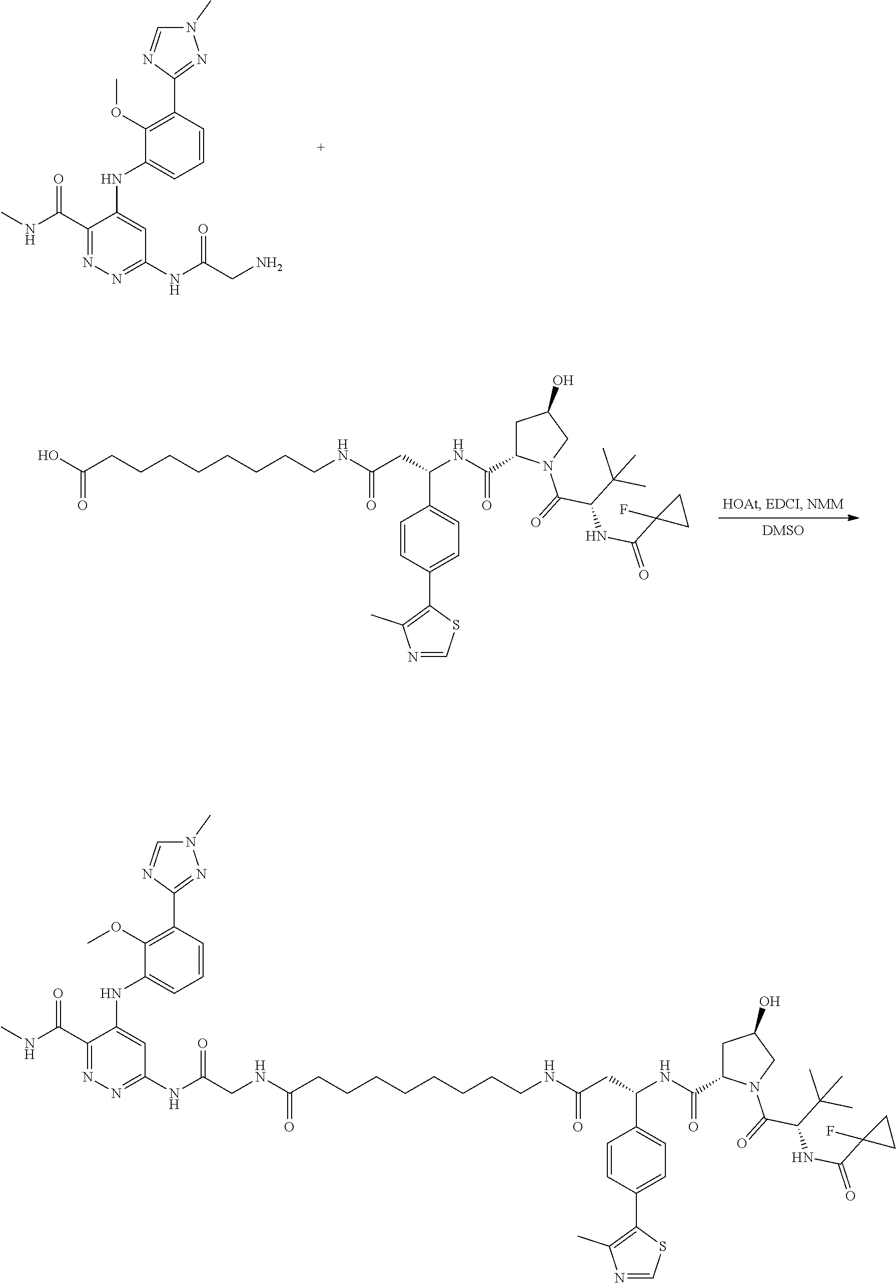 [SMILES: CNC(=O)c1nnc(NC(=O)CN)cc1Nc1cccc(-c2ncn(C)n2)c1OC ; Cc1ncsc1-c1ccc([C@H](CC(=O)NCCCCCCCCC(=O)O)NC(=O)[C@@H]2C[C@@H](O)CN2C(=O)[C@@H](NC(=O)C2(F)CC2)C(C)(C)C)cc1 ; CNC(=O)c1nnc(NC(=O)CNC(=O)CCCCCCCCNC(=O)C[C@H](NC(=O)[C@@H]2C[C@@H](O)CN2C(=O)[C@@H](NC(=O)C2(F)CC2)C(C)(C)C)c2ccc(-c3scnc3C)cc2)cc1Nc1cccc(-c2ncn(C)n2)c1OC]

CPD-093 was synthesized following the same procedure for preparing CPD-042 (2.42 mg, yield: 17.8%). MS (ESI) m/z=1124.2 [M+H]⁺.

### Example 137: 6-(2-(10-((S)-3-((2S,4R)-1-((S)-2-(1-Fluorocyclopropane-1-carboxamido)-3,3-dimethylbutanoyl)-4-hydroxypyrrolidine-2-carboxamido)-3-(4-(4-methylthiazol-5-yl)phenyl)propanamido)decanamido)acetamido)-4-((2-methoxy-3-(1-methyl-1H-1,2,4-triazol-3-yl)phenyl)amino)-N-methylpyridazine-3-carboxamide (CPD-094)

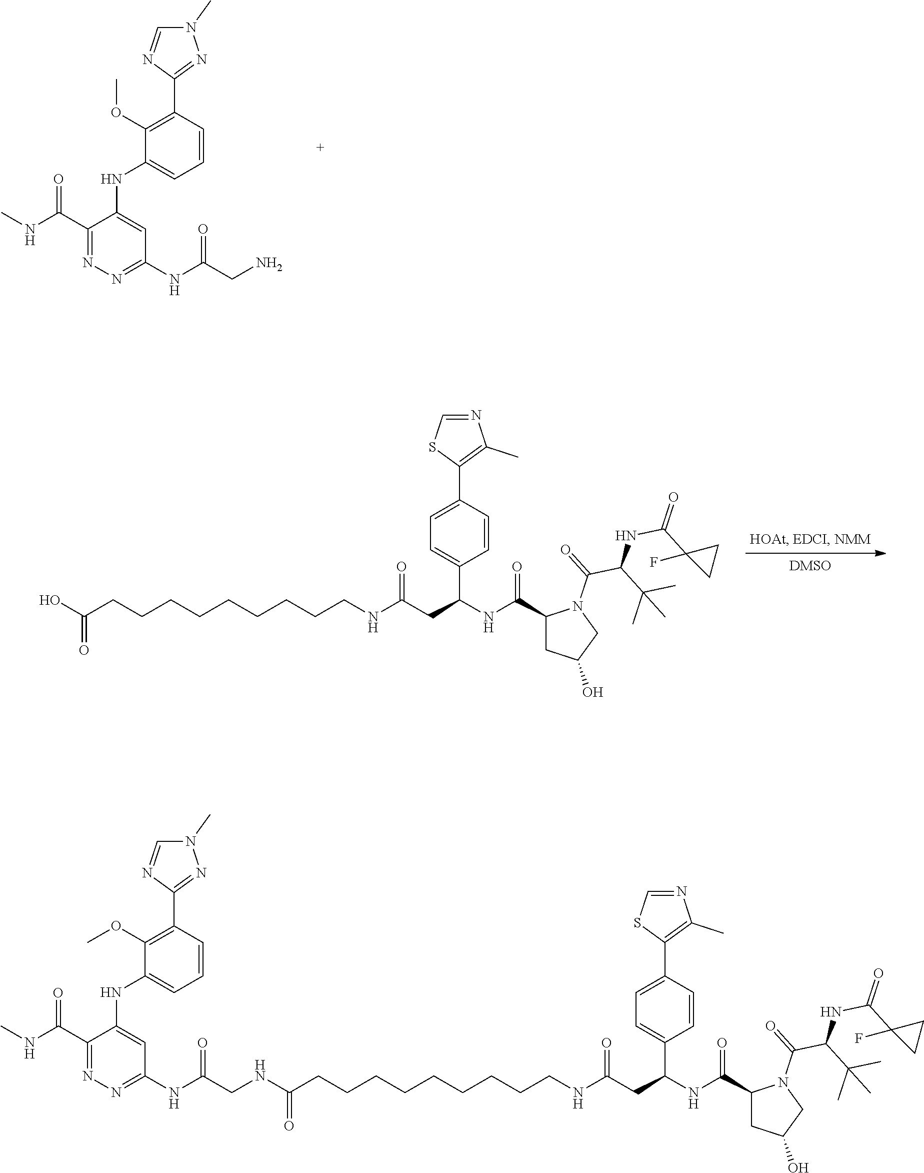 [SMILES: CNC(=O)c1nnc(NC(=O)CN)cc1Nc1cccc(-c2ncn(C)n2)c1OC ; Cc1ncsc1-c1ccc([C@H](CC(=O)NCCCCCCCCCC(=O)O)NC(=O)[C@@H]2C[C@@H](O)CN2C(=O)[C@@H](NC(=O)C2(F)CC2)C(C)(C)C)cc1 ; CNC(=O)c1nnc(NC(=O)CNC(=O)CCCCCCCCCNC(=O)C[C@H](NC(=O)[C@@H]2C[C@@H](O)CN2C(=O)[C@@H](NC(=O)C2(F)CC2)C(C)(C)C)c2ccc(-c3scnc3C)cc2)cc1Nc1cccc(-c2ncn(C)n2)c1OC]

CPD-094 was synthesized following the same procedure for preparing CPD-042 (1.53 mg, yield: 11.1%). MS (ESI) m/z=1138.2 [M+H]⁺.

### Example 138: 6-(2-(11-((S)-3-((2S,4R)-1-((S)-2-(1-Fluorocyclopropane-1-carboxamido)-3,3-dimethylbutanoyl)-4-hydroxypyrrolidine-2-carboxamido)-3-(4-(4-methylthiazol-5-yl)phenyl)propanamido)undecanamido)acetamido)-4-((2-methoxy-3-(1-methyl-1H-1,2,4-triazol-3-yl)phenyl)amino)-N-methylpyridazine-3-carboxamide (CPD-095)

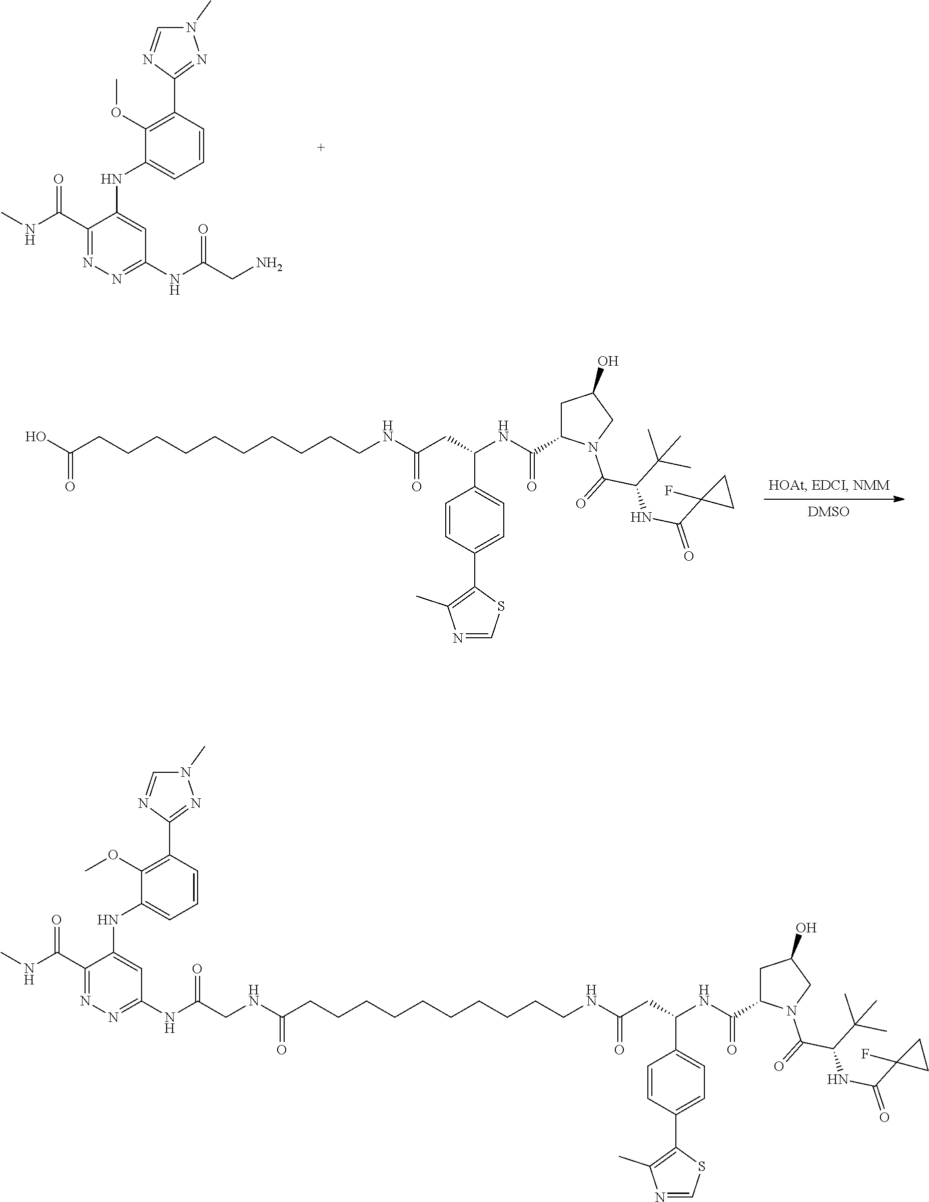 [SMILES: CNC(=O)c1nnc(NC(=O)CN)cc1Nc1cccc(-c2ncn(C)n2)c1OC ; Cc1ncsc1-c1ccc([C@H](CC(=O)NCCCCCCCCCCC(=O)O)NC(=O)[C@@H]2C[C@@H](O)CN2C(=O)[C@@H](NC(=O)C2(F)CC2)C(C)(C)C)cc1 ; CNC(=O)c1nnc(NC(=O)CNC(=O)CCCCCCCCCCNC(=O)C[C@H](NC(=O)[C@@H]2C[C@@H](O)CN2C(=O)[C@@H](NC(=O)C2(F)CC2)C(C)(C)C)c2ccc(-c3scnc3C)cc2)cc1Nc1cccc(-c2ncn(C)n2)c1OC]

CPD-095 was synthesized following the same procedure for preparing CPD-042 (2.89 mg, yield: 20.7%). MS (ESI) m/z=1152.2 [M+H]⁺.

### Example 139: 6-((5-(4-(4-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-4-oxobutanoyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-096)

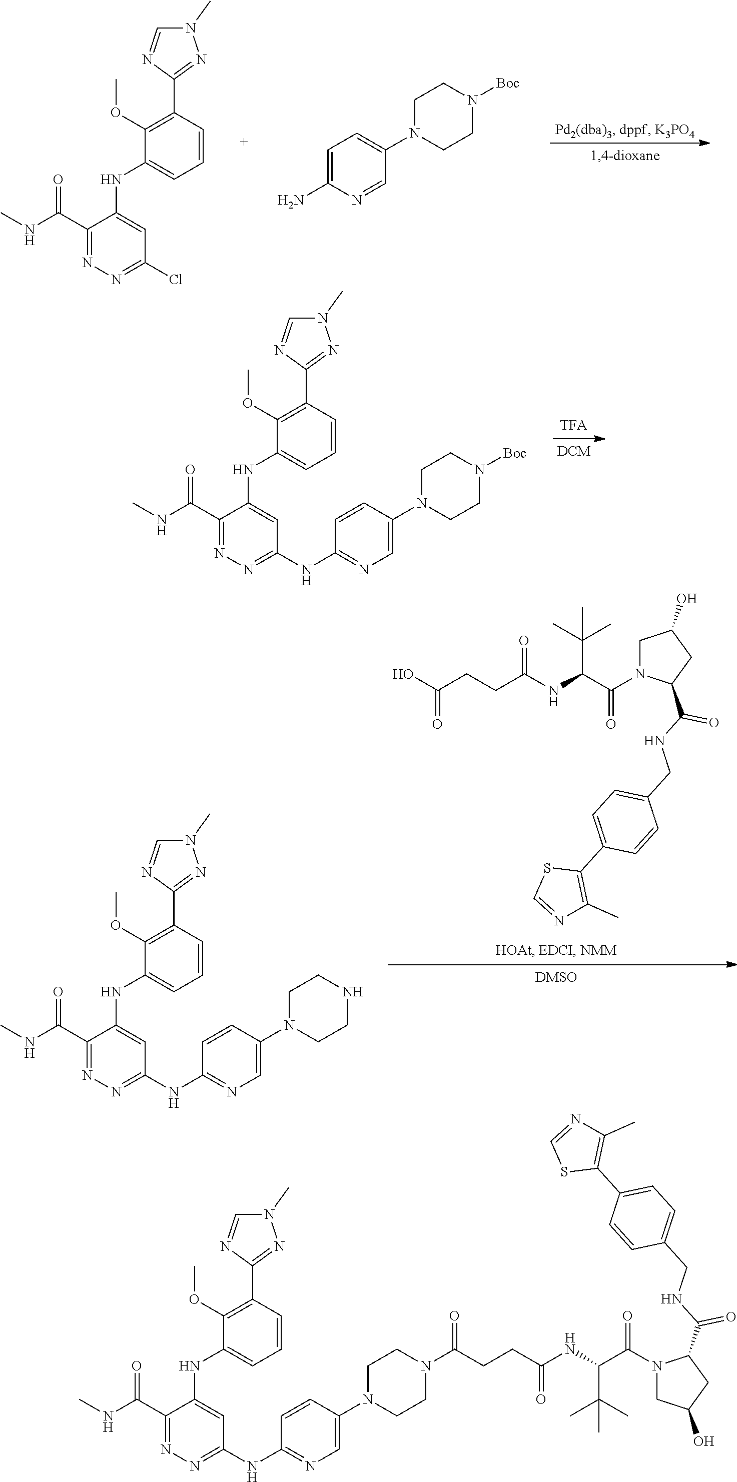

### Step 1. Synthesis of tert-butyl 4-(6-((5-((2-methoxy-3-(1-methyl-JH-1,2,4-triazol-3-yl)phenyl)amino)-6-(methylcarbamoyl)pyridazin-3-yl)amino)pyridin-3-yl)piperazine-1-carboxylate

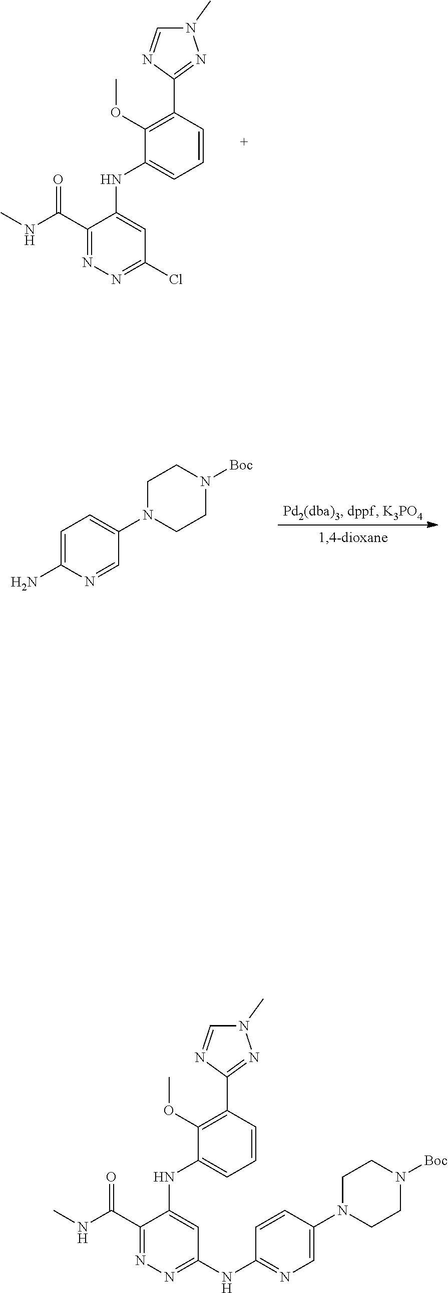

A mixture of 6-chloro-4-((2-methoxy-3-(1-methyl-1H-1,2,4-triazol-3-yl)phenyl)amino)-N-methylpyridazine-3-carboxamide (450 mg, 1.2 mmol) and K₃PO₄ (763 mg, 3.6 mmol) in 1,4-dioxane (6 mL) were added tert-butyl 4-(6-aminopyridin-3-yl)piperazine-1-carboxylate (500 mg, 1.8 mmol), Pd₂(dba)₃ (10 mg, 0.01 mmol) and dppf (11 mg, 0.02 mmol). The resulting mixture was stirred at 100° C. under N₂ for 4 h, before the reaction mixture was quenched with water and extracted with EtOAc (30 mL×3). The combined organic layers were washed with brine (30 mL×2), dried over Na₂SO₄, filtered, and concentrated under reduced pressure. The residue was purified by reverse-phase chromatography to give the title compound (389 mg, yield: 52.5%) as a light-yellow oil. MS (ESI) m/z=616.6 [M+H]⁺.

### Step 2. Synthesis of 4-((2-methoxy-3-(1-methyl-1H-1,2,4-triazol-3-yl)phenyl)amino)-N-methyl-6-((5-(piperazin-1-yl)pyridin-2-yl)amino)pyridazine-3-carboxamide

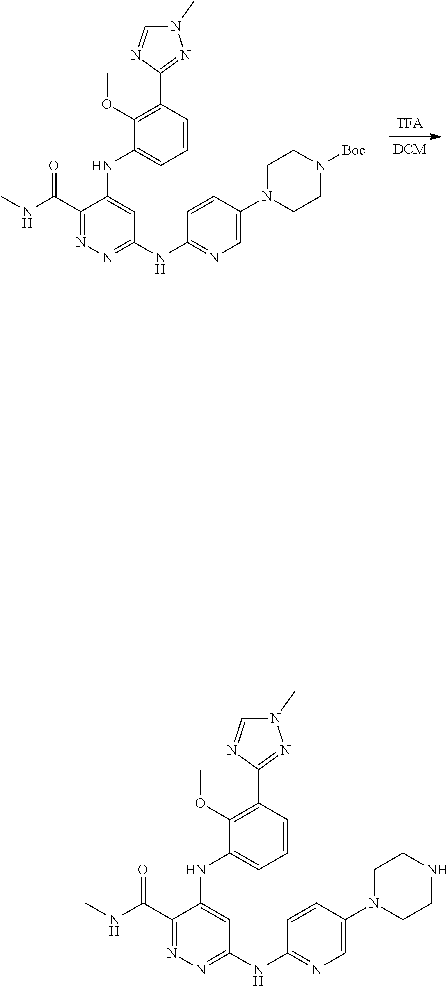

To a mixture of tert-butyl 4-(6-((5-((2-methoxy-3-(1-methyl-1H-1,2,4-triazol-3-yl)phenyl)amino)-6-(methylcarbamoyl)pyridazin-3-yl)amino)pyridin-3-yl)piperazine-1-carboxylate (389 mg, 0.63 mmol) in DCM (5 mL) was added TFA (5 mL). The resulting mixture was stirred at 25° C. for 16 h, before it was concentrated. The residue was purified by reverse-phase chromatography to give the title compound (307 mg, yield: 94.5%) as a light-yellow solid. MS (ESI) m/z=516.6 [M+H]⁺.

### Step 3. Synthesis of 6-((5-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-4-oxobutanoyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide

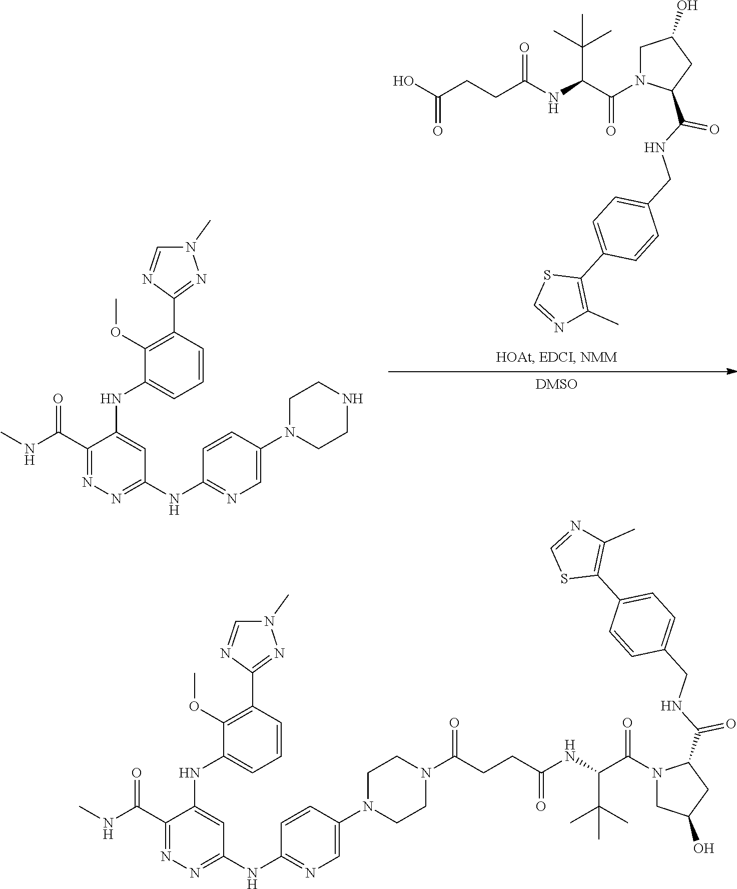 [SMILES: CNC(=O)c1nnc(Nc2ccc(N3CCNCC3)cn2)cc1Nc1cccc(-c2ncn(C)n2)c1OC ; Cc1ncsc1-c1ccc(CNC(=O)[C@@H]2C[C@@H](O)CN2C(=O)[C@@H](NC(=O)CCC(=O)O)C(C)(C)C)cc1 ; CNC(=O)c1nnc(Nc2ccc(N3CCN(C(=O)CCC(=O)N[C@H](C(=O)N4C[C@H](O)C[C@H]4C(=O)NCc4ccc(-c5scnc5C)cc4)C(C)(C)C)CC3)cn2)cc1Nc1cccc(-c2ncn(C)n2)c1OC]

CPD-096 was synthesized following the same procedure for preparing CPD-042 (3.42 mg, yield: 34.3%). MS (ESI) m/z=1029.0 [M+H]⁺.

### Example 140: 6-((5-(4-(5-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-5-oxopentanoyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-097)

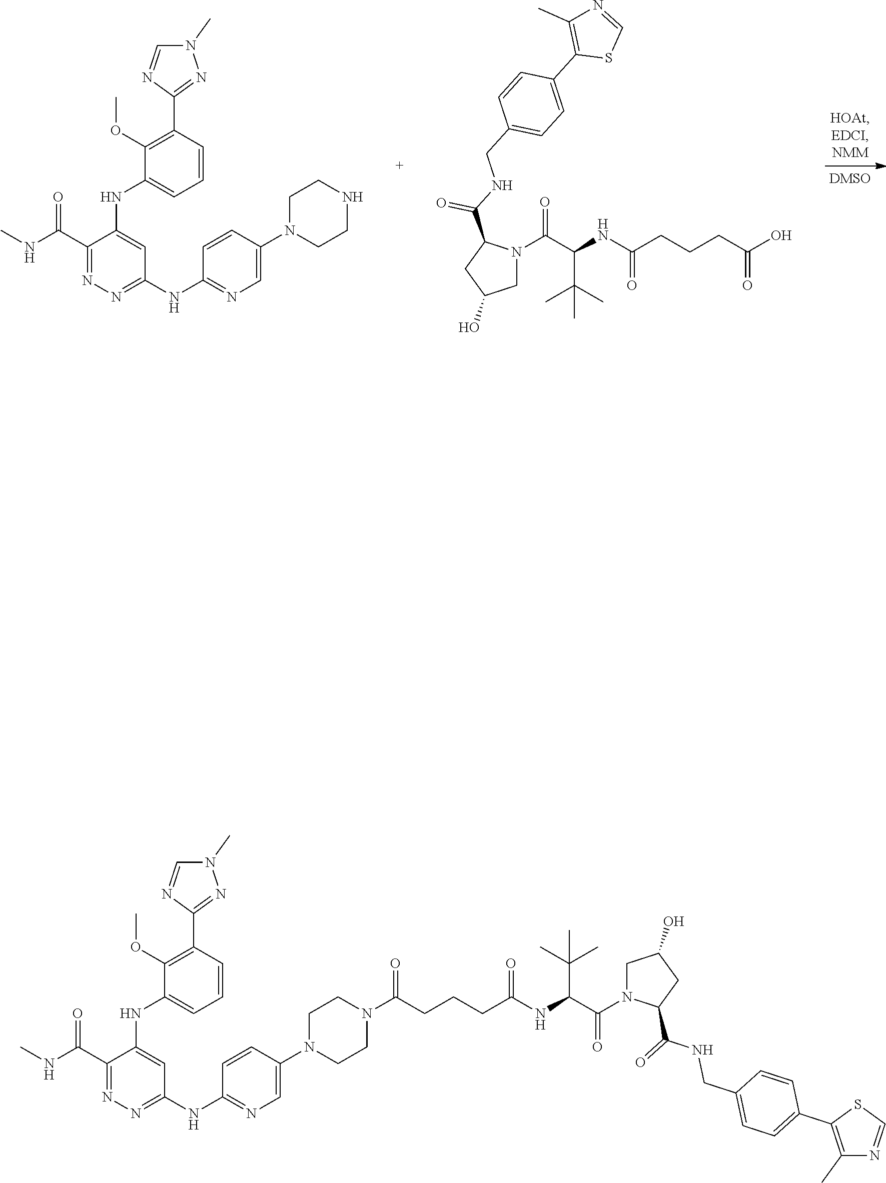 [SMILES: CNC(=O)c1nnc(Nc2ccc(N3CCNCC3)cn2)cc1Nc1cccc(-c2ncn(C)n2)c1OC ; Cc1ncsc1-c1ccc(CNC(=O)[C@@H]2C[C@@H](O)CN2C(=O)[C@@H](NC(=O)CCCC(=O)O)C(C)(C)C)cc1 ; CNC(=O)c1nnc(Nc2ccc(N3CCN(C(=O)CCCC(=O)N[C@H](C(=O)N4C[C@H](O)C[C@H]4C(=O)NCc4ccc(-c5scnc5C)cc4)C(C)(C)C)CC3)cn2)cc1Nc1cccc(-c2ncn(C)n2)c1OC]

CPD-097 was synthesized following the same procedure for preparing CPD-096 (2.04 mg, yield: 20.2%). MS (ESI) m/z=1043.0 [M+H]⁺.

### Example 141: 6-((5-(4-(6-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-6-oxohexanoyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-098)

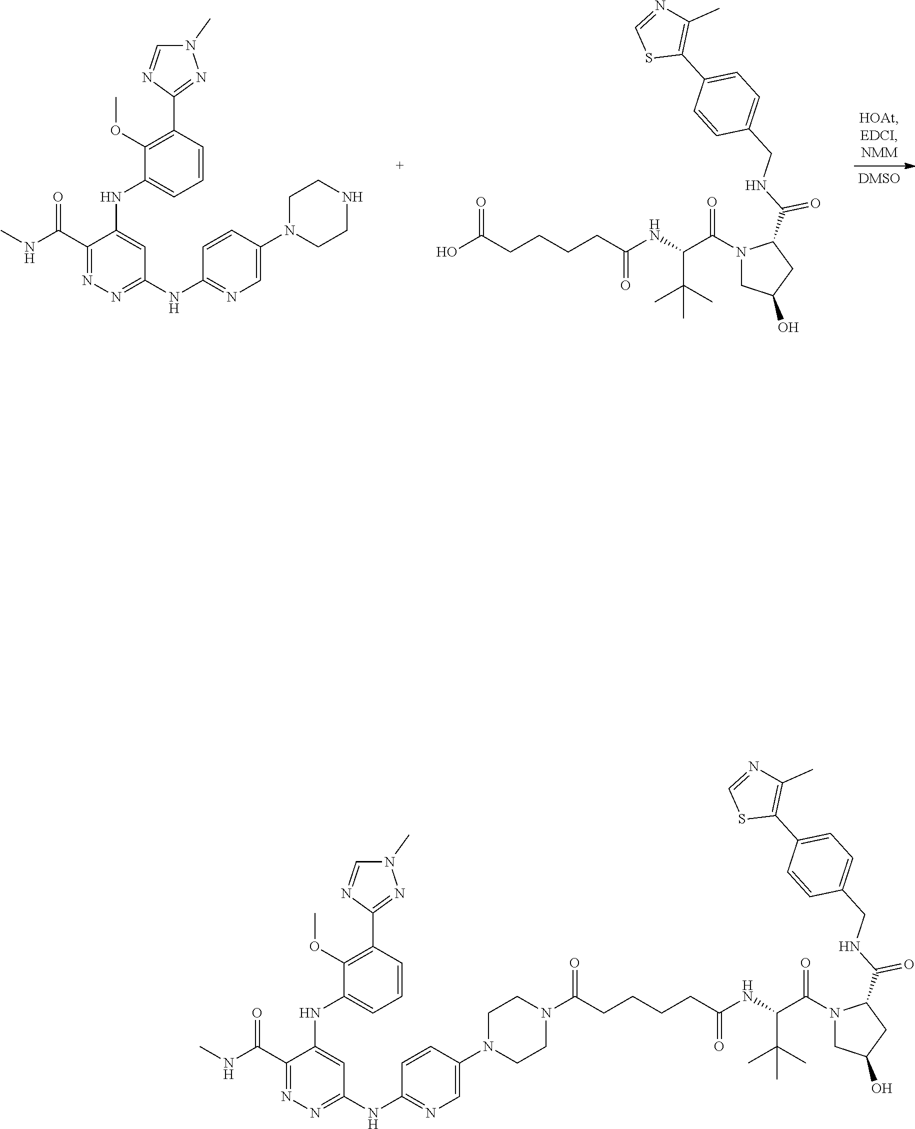 [SMILES: CNC(=O)c1nnc(Nc2ccc(N3CCNCC3)cn2)cc1Nc1cccc(-c2ncn(C)n2)c1OC ; Cc1ncsc1-c1ccc(CNC(=O)[C@@H]2C[C@@H](O)CN2C(=O)[C@@H](NC(=O)CCCCC(=O)O)C(C)(C)C)cc1 ; CNC(=O)c1nnc(Nc2ccc(N3CCN(C(=O)CCCCC(=O)N[C@H](C(=O)N4C[C@H](O)C[C@H]4C(=O)NCc4ccc(-c5scnc5C)cc4)C(C)(C)C)CC3)cn2)cc1Nc1cccc(-c2ncn(C)n2)c1OC]

CPD-098 was synthesized following the same procedure for preparing CPD-096 (2.07 mg, yield: 20.2%). MS (ESI) m/z=1057.1 [M+H]⁺.

### Example 142: 6-((5-(4-(7-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-7-oxoheptanoyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-099)

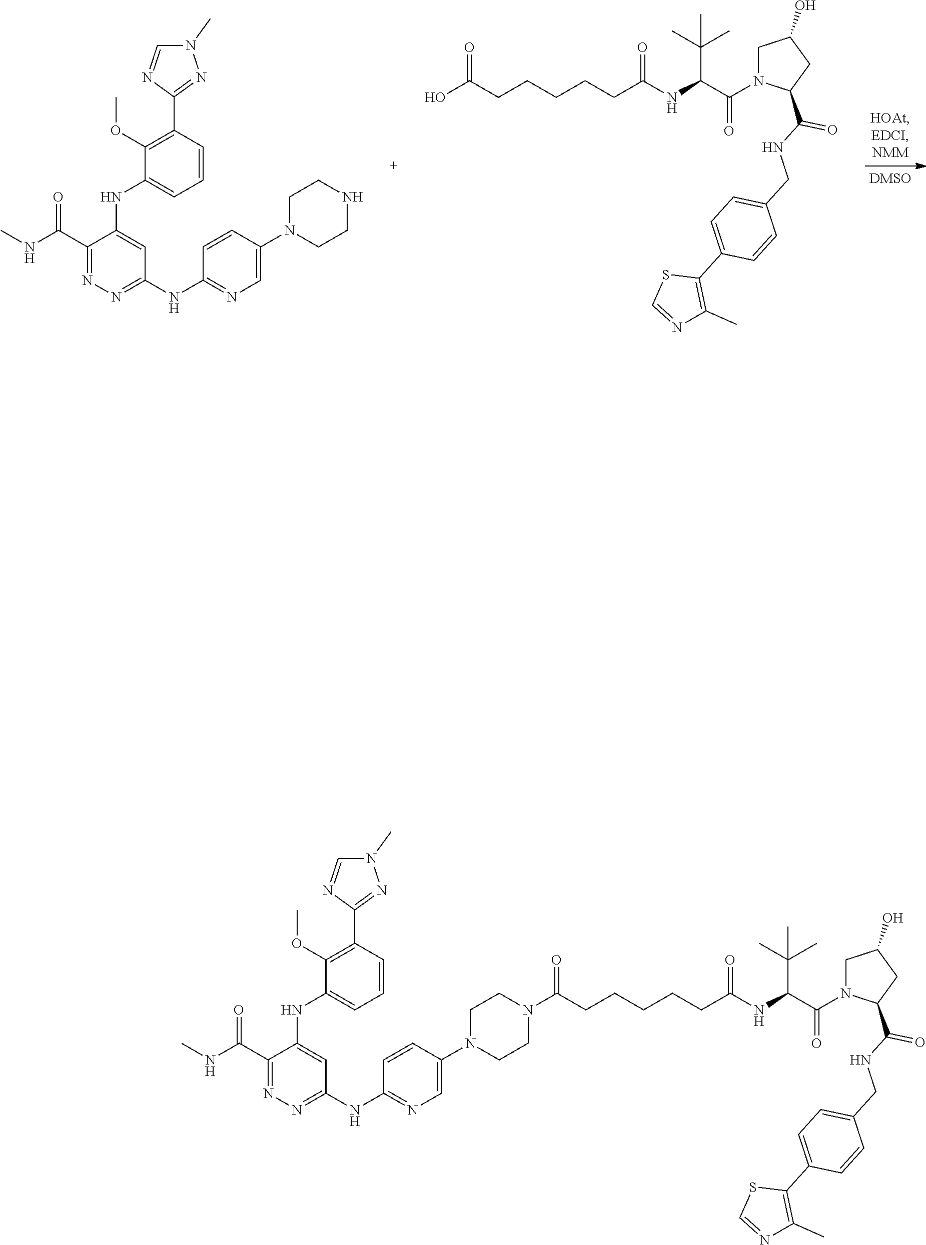 [SMILES: CNC(=O)c1nnc(Nc2ccc(N3CCNCC3)cn2)cc1Nc1cccc(-c2ncn(C)n2)c1OC ; Cc1ncsc1-c1ccc(CNC(=O)[C@@H]2C[C@@H](O)CN2C(=O)[C@@H](NC(=O)CCCCCC(=O)O)C(C)(C)C)cc1 ; CNC(=O)c1nnc(Nc2ccc(N3CCN(C(=O)CCCCCC(=O)N[C@H](C(=O)N4C[C@H](O)C[C@H]4C(=O)NCc4ccc(-c5scnc5C)cc4)C(C)(C)C)CC3)cn2)cc1Nc1cccc(-c2ncn(C)n2)c1OC]

CPD-099 was synthesized following the same procedure for preparing CPD-096 (2.98 mg, yield: 28.7%). MS (ESI) m/z=1071.1 [M+H]⁺.

### Example 142: 6-((5-(4-(8-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-8-oxooctanoyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-100)

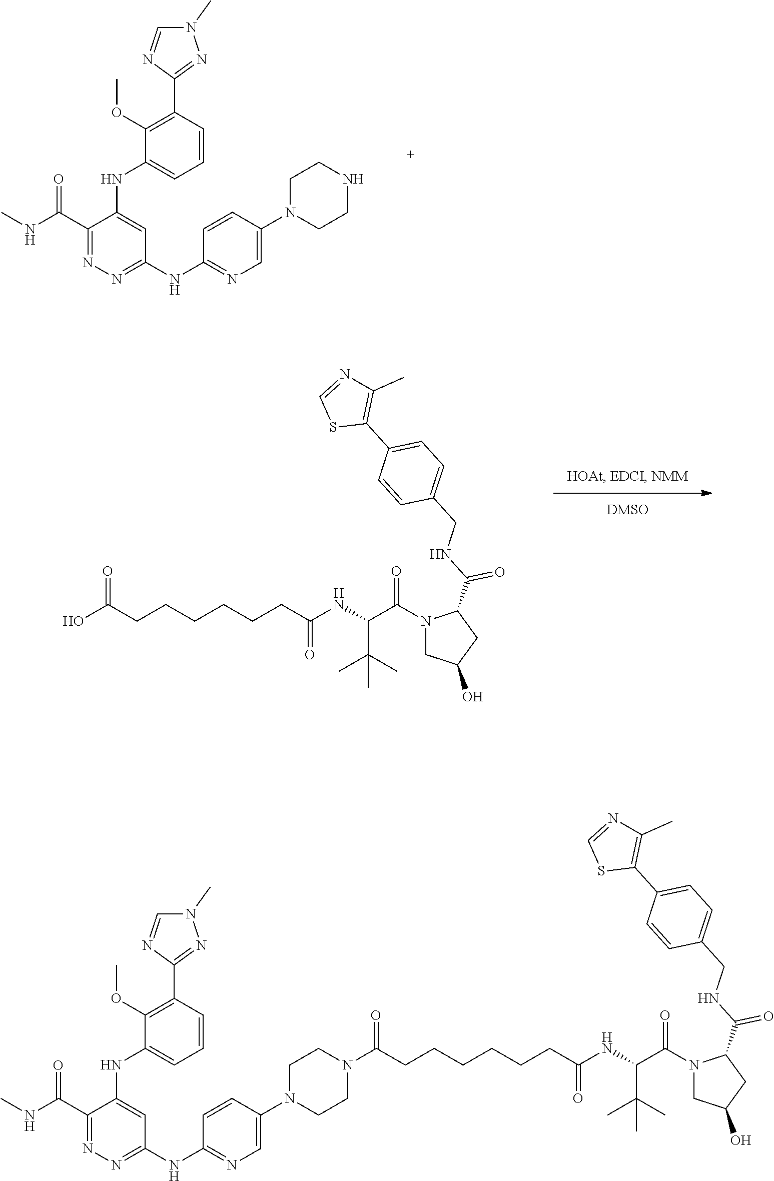 [SMILES: CNC(=O)c1nnc(Nc2ccc(N3CCNCC3)cn2)cc1Nc1cccc(-c2ncn(C)n2)c1OC ; Cc1ncsc1-c1ccc(CNC(=O)[C@@H]2C[C@@H](O)CN2C(=O)[C@@H](NC(=O)CCCCCCC(=O)O)C(C)(C)C)cc1 ; CNC(=O)c1nnc(Nc2ccc(N3CCN(C(=O)CCCCCCC(=O)N[C@H](C(=O)N4C[C@H](O)C[C@H]4C(=O)NCc4ccc(-c5scnc5C)cc4)C(C)(C)C)CC3)cn2)cc1Nc1cccc(-c2ncn(C)n2)c1OC]

CPD-100 was synthesized following the same procedure for preparing CPD-096 (3.23 mg, yield: 30.8%). MS (ESI) m/z=1085.1 [M+H]⁺.

### Example 143: 6-(2-(2-(2-(((2S,4R)-1-((S)-2-(1-Fluorocyclopropane-1-carboxamido)-3,3-dimethylbutanoyl)-4-hydroxypyrrolidine-2-carboxamido)methyl)-5-(4-methylthiazol-5-yl)phenoxy)acetamido)acetamido)-4-((2-methoxy-3-(1-methyl-1H-1,2,4-triazol-3-yl)phenyl)amino)-N-methylpyridazine-3-carboxamide (CPD-101)

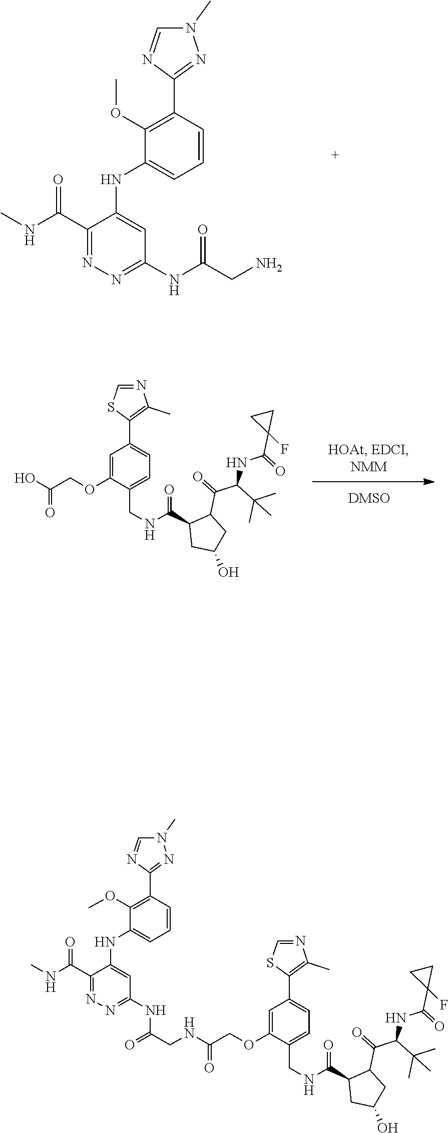 [SMILES: CNC(=O)c1nnc(NC(=O)CN)cc1Nc1cccc(-c2ncn(C)n2)c1OC ; Cc1ncsc1-c1ccc(CNC(=O)[C@@H]2C[C@@H](O)CC2C(=O)[C@@H](NC(=O)C2(F)CC2)C(C)(C)C)c(OCC(=O)O)c1 ; CNC(=O)c1nnc(NC(=O)CNC(=O)COc2cc(-c3scnc3C)ccc2CNC(=O)[C@@H]2C[C@@H](O)CC2C(=O)[C@@H](NC(=O)C2(F)CC2)C(C)(C)C)cc1Nc1cccc(-c2ncn(C)n2)c1OC]

CPD-101 was synthesized following the same procedure for preparing CPD-042 (3.5 mg, yield: 29.3%). MS (ESI) m/z=984.9 [M+H]⁺.

### Example 144: 6-(2-(3-(2-(((2S,4R)-1-((S)-2-(1-Fluorocyclopropane-1-carboxamido)-3,3-dimethylbutanoyl)-4-hydroxypyrrolidine-2-carboxamido)methyl)-5-(4-methylthiazol-5-yl)phenoxy)propanamido)acetamido)-4-((2-methoxy-3-(1-methyl-1H-1,2,4-triazol-3-yl)phenyl)amino)-N-methylpyridazine-3-carboxamide (CPD-102)

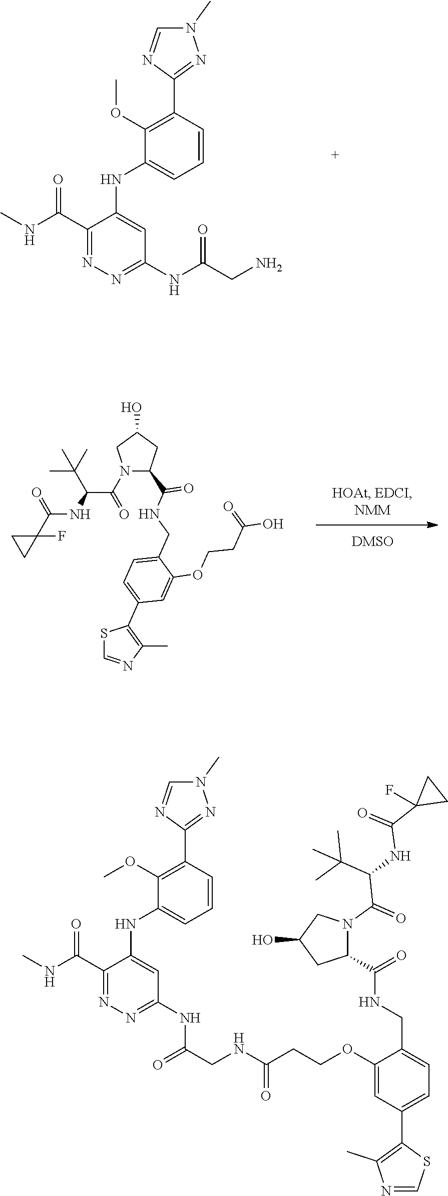 [SMILES: CNC(=O)c1nnc(NC(=O)CN)cc1Nc1cccc(-c2ncn(C)n2)c1OC ; Cc1ncsc1-c1ccc(CNC(=O)[C@@H]2C[C@@H](O)CN2C(=O)[C@@H](NC(=O)C2(F)CC2)C(C)(C)C)c(OCCC(=O)O)c1 ; CNC(=O)c1nnc(NC(=O)CNC(=O)CCOc2cc(-c3scnc3C)ccc2CNC(=O)[C@@H]2C[C@@H](O)CN2C(=O)[C@@H](NC(=O)C2(F)CC2)C(C)(C)C)cc1Nc1cccc(-c2ncn(C)n2)c1OC]

CPD-102 was synthesized following the same procedure for preparing CPD-042 (2.87 mg, yield: 23.7%). MS (ESI) m/z=999.0 [M+H]⁺.

### Example 145: 6-(2-(4-(2-(((2S,4R)-1-((S)-2-(1-Fluorocyclopropane-1-carboxamido)-3,3-dimethylbutanoyl)-4-hydroxypyrrolidine-2-carboxamido)methyl)-5-(4-methylthiazol-5-yl)phenoxy)butanamido)acetamido)-4-((2-methoxy-3-(1-methyl-1H-1,2,4-triazol-3-yl)phenyl)amino)-N-methylpyridazine-3-carboxamide (CPD-103)

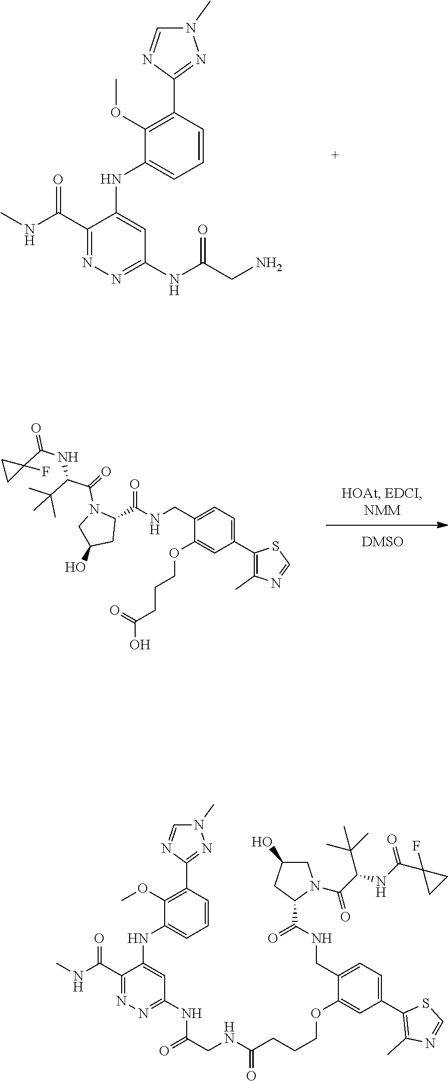 [SMILES: CNC(=O)c1nnc(NC(=O)CN)cc1Nc1cccc(-c2ncn(C)n2)c1OC ; Cc1ncsc1-c1ccc(CNC(=O)[C@@H]2C[C@@H](O)CN2C(=O)[C@@H](NC(=O)C2(F)CC2)C(C)(C)C)c(OCCCC(=O)O)c1 ; CNC(=O)c1nnc(NC(=O)CNC(=O)CCCOc2cc(-c3scnc3C)ccc2CNC(=O)[C@@H]2C[C@@H](O)CN2C(=O)[C@@H](NC(=O)C2(F)CC2)C(C)(C)C)cc1Nc1cccc(-c2ncn(C)n2)c1OC]

CPD-103 was synthesized following the same procedure for preparing CPD-042 (5.54 mg, yield: 45.0%). MS (ESI) m/z=1012.9 [M+H]⁺.

### Example 146: 6-(2-(5-(2-(((2S,4R)-1-((S)-2-(1-Fluorocyclopropane-1-carboxamido)-3,3-dimethylbutanoyl)-4-hydroxypyrrolidine-2-carboxamido)methyl)-5-(4-methylthiazol-5-yl)phenoxy)pentanamido)acetamido)-4-((2-methoxy-3-(1-methyl-1H-1,2,4-triazol-3-yl)phenyl)amino)-N-methylpyridazine-3-carboxamide (CPD-104)

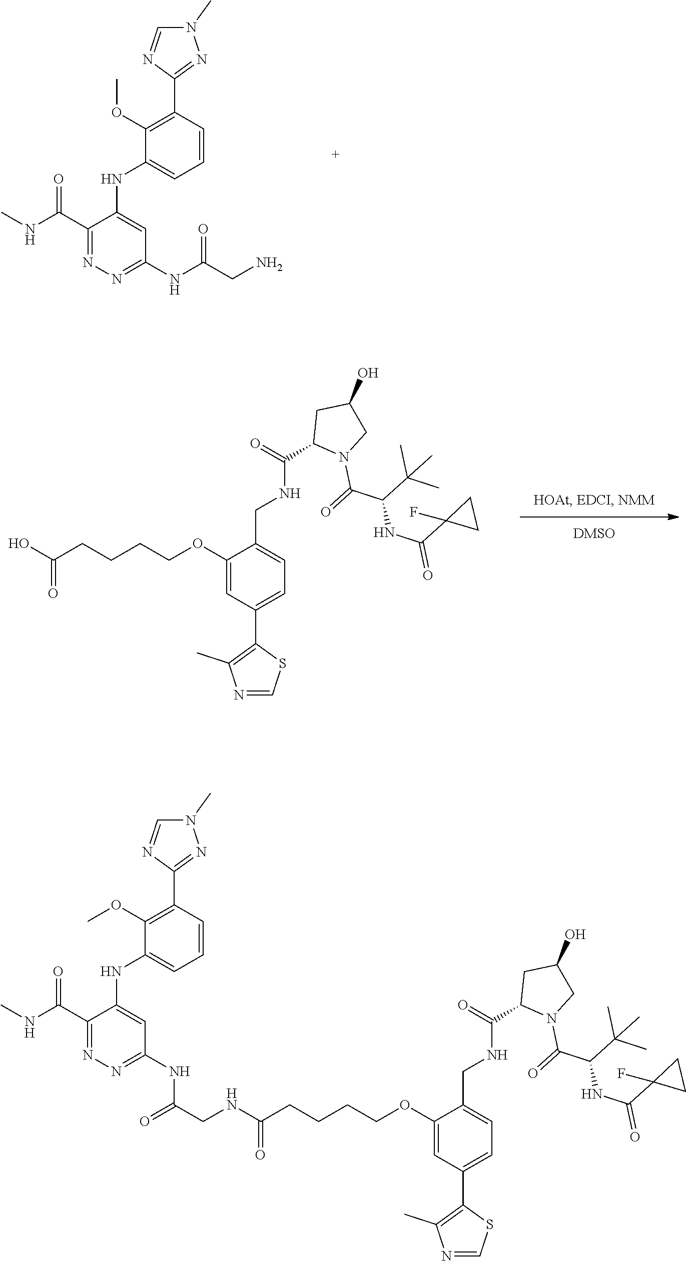 [SMILES: CNC(=O)c1nnc(NC(=O)CN)cc1Nc1cccc(-c2ncn(C)n2)c1OC ; Cc1ncsc1-c1ccc(CNC(=O)[C@@H]2C[C@@H](O)CN2C(=O)[C@@H](NC(=O)C2(F)CC2)C(C)(C)C)c(OCCCCC(=O)O)c1 ; CNC(=O)c1nnc(NC(=O)CNC(=O)CCCCOc2cc(-c3scnc3C)ccc2CNC(=O)[C@@H]2C[C@@H](O)CN2C(=O)[C@@H](NC(=O)C2(F)CC2)C(C)(C)C)cc1Nc1cccc(-c2ncn(C)n2)c1OC]

CPD-104 was synthesized following the same procedure for preparing CPD-042 (6.41 mg, yield: 51.4%). MS (ESI) m/z=1027.0 [M+H]⁺.

### Example 147: 6-(2-(6-(2-(((2S,4R)-1-((S)-2-(1-Fluorocyclopropane-1-carboxamido)-3,3-dimethylbutanoyl)-4-hydroxypyrrolidine-2-carboxamido)methyl)-5-(4-methylthiazol-5-yl)phenoxy)hexanamido)acetamido)-4-((2-methoxy-3-(1-methyl-1H-1,2,4-triazol-3-yl)phenyl)amino)-N-methylpyridazine-3-carboxamide (CPD-105)

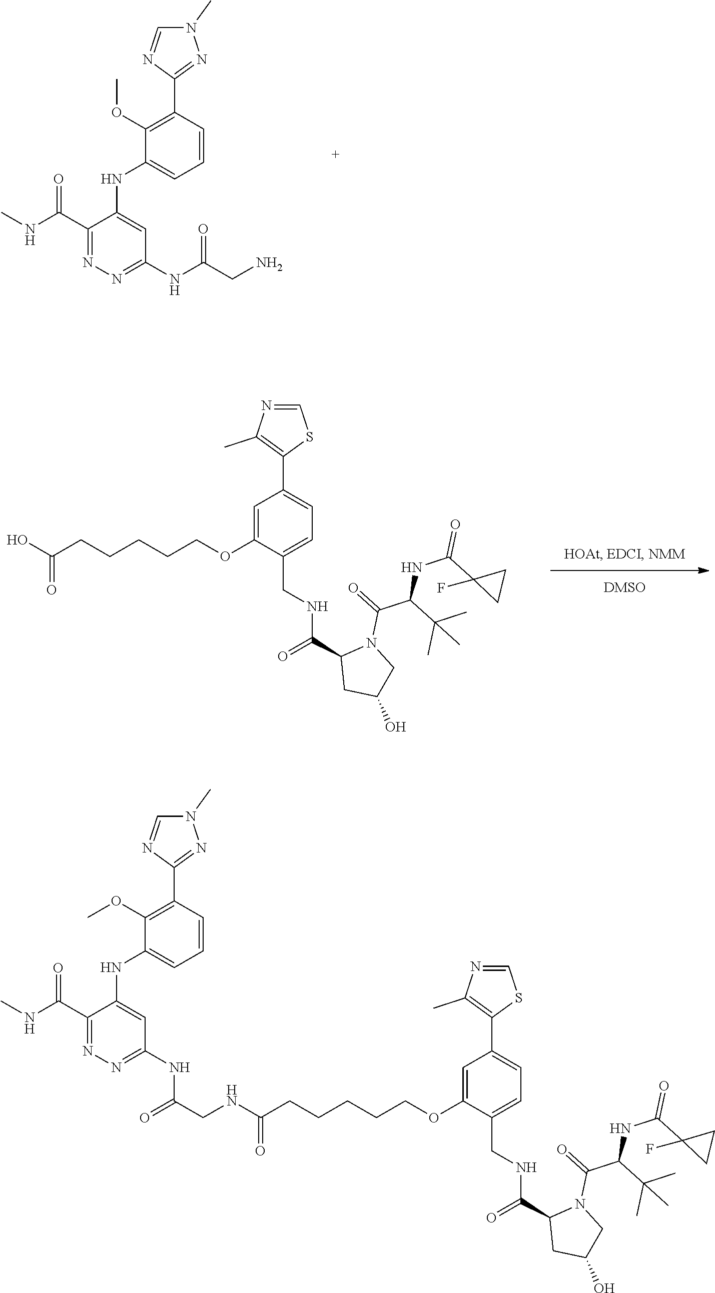 [SMILES: CNC(=O)c1nnc(NC(=O)CN)cc1Nc1cccc(-c2ncn(C)n2)c1OC ; Cc1ncsc1-c1ccc(CNC(=O)[C@@H]2C[C@@H](O)CN2C(=O)[C@@H](NC(=O)C2(F)CC2)C(C)(C)C)c(OCCCCCC(=O)O)c1 ; CNC(=O)c1nnc(NC(=O)CNC(=O)CCCCCOc2cc(-c3scnc3C)ccc2CNC(=O)[C@@H]2C[C@@H](O)CN2C(=O)[C@@H](NC(=O)C2(F)CC2)C(C)(C)C)cc1Nc1cccc(-c2ncn(C)n2)c1OC]

CPD-105 was synthesized following the same procedure for preparing CPD-042 (3.66 mg, yield: 29.0%). MS (ESI) m/z=1041.0 [M+H]⁺.

### Example 148: 6-(2-(7-(2-(((2S,4R)-1-((S)-2-(1-Fluorocyclopropane-1-carboxamido)-3,3-dimethylbutanoyl)-4-hydroxypyrrolidine-2-carboxamido)methyl)-5-(4-methylthiazol-5-yl)phenoxy)heptanamido)acetamido)-4-((2-methoxy-3-(1-methyl-1H-1,2,4-triazol-3-yl)phenyl)amino)-N-methylpyridazine-3-carboxamide (CPD-106)

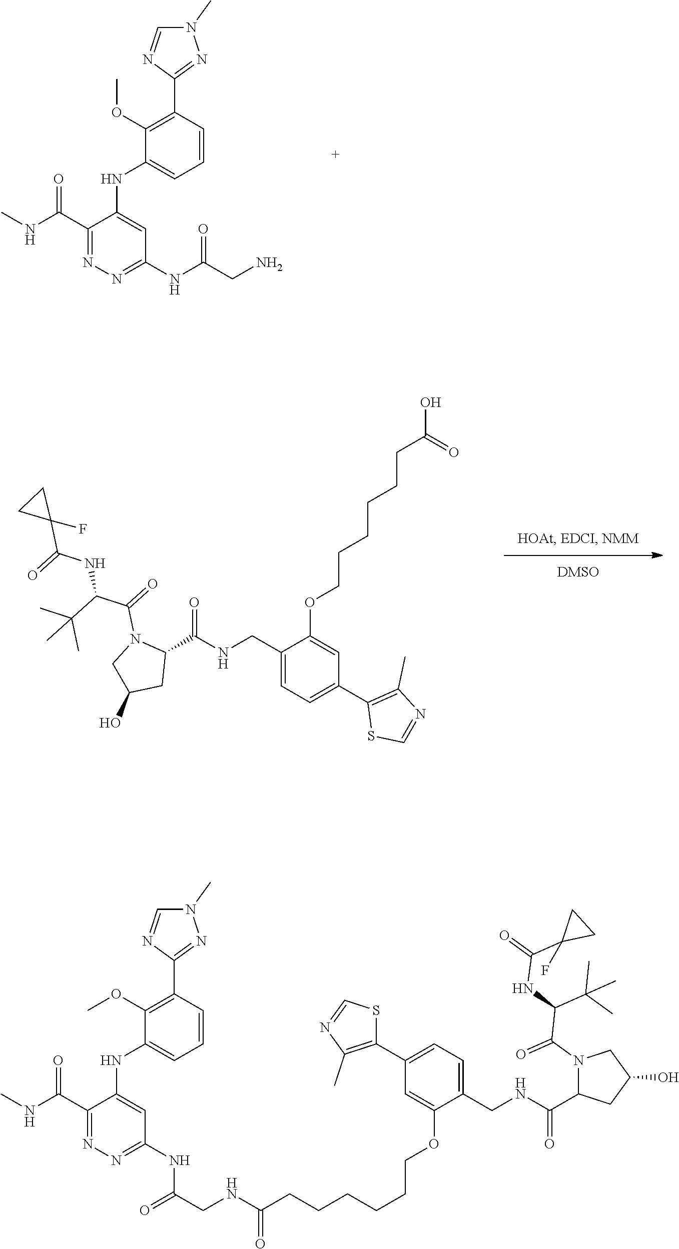 [SMILES: CNC(=O)c1nnc(NC(=O)CN)cc1Nc1cccc(-c2ncn(C)n2)c1OC ; Cc1ncsc1-c1ccc(CNC(=O)[C@@H]2C[C@@H](O)CN2C(=O)[C@@H](NC(=O)C2(F)CC2)C(C)(C)C)c(OCCCCCCC(=O)O)c1 ; CNC(=O)c1nnc(NC(=O)CNC(=O)CCCCCCOc2cc(-c3scnc3C)ccc2CNC(=O)C2C[C@@H](O)CN2C(=O)[C@@H](NC(=O)C2(F)CC2)C(C)(C)C)cc1Nc1cccc(-c2ncn(C)n2)c1OC]

CPD-106 was synthesized following the same procedure for preparing CPD-042 (5.4 mg, yield: 42.2%). MS (ESI) m/z=1055.1 [M+H]⁺.

### Example 149: 6-(2-(8-(2-(((2S,4R)-1-((S)-2-(1-Fluorocyclopropane-1-carboxamido)-3,3-dimethylbutanoyl)-4-hydroxypyrrolidine-2-carboxamido)methyl)-5-(4-methylthiazol-5-yl)phenoxy)octanamido)acetamido)-4-((2-methoxy-3-(1-methyl-1H-1,2,4-triazol-3-yl)phenyl)amino)-N-methylpyridazine-3-carboxamide (CPD-107)

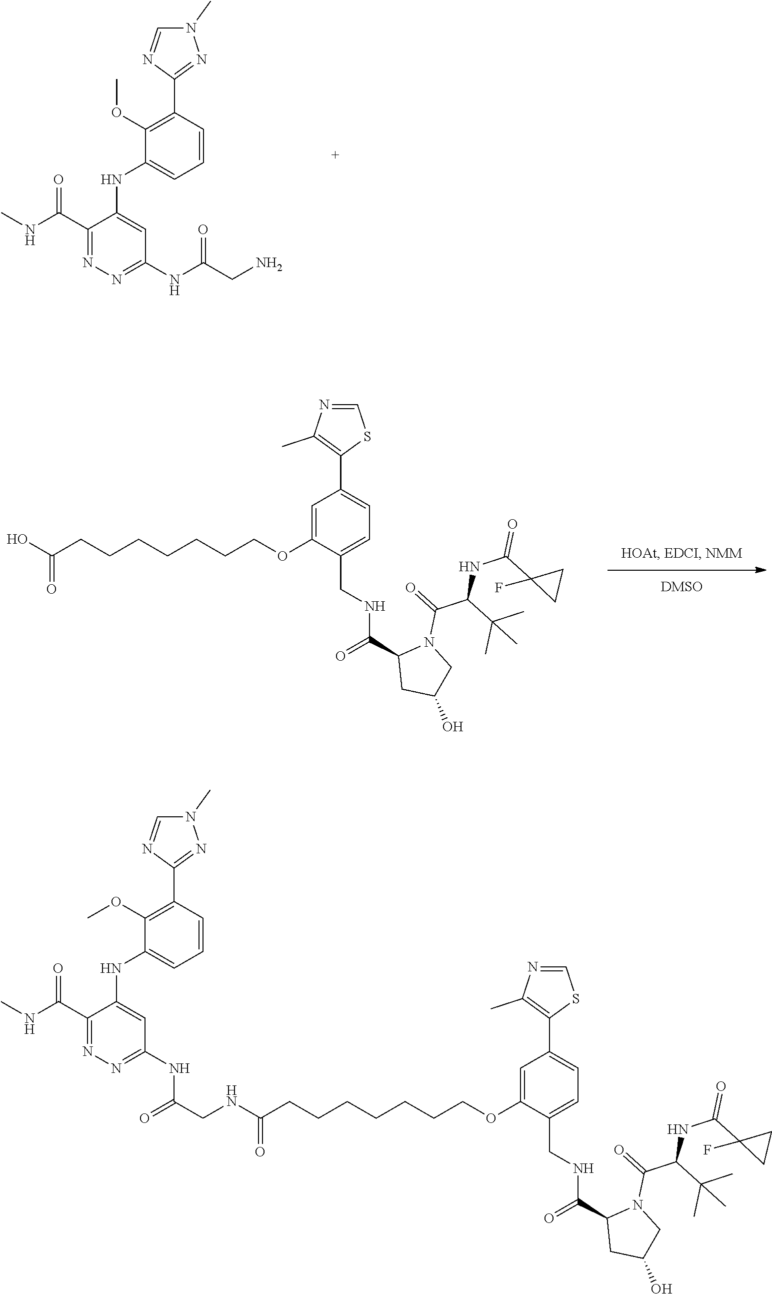 [SMILES: CNC(=O)c1nnc(NC(=O)CN)cc1Nc1cccc(-c2ncn(C)n2)c1OC ; Cc1ncsc1-c1ccc(CNC(=O)[C@@H]2C[C@@H](O)CN2C(=O)[C@@H](NC(=O)C2(F)CC2)C(C)(C)C)c(OCCCCCCCC(=O)O)c1 ; CNC(=O)c1nnc(NC(=O)CNC(=O)CCCCCCCOc2cc(-c3scnc3C)ccc2CNC(=O)[C@@H]2C[C@@H](O)CN2C(=O)[C@@H](NC(=O)C2(F)CC2)C(C)(C)C)cc1Nc1cccc(-c2ncn(C)n2)c1OC]

CPD-107 was synthesized following the same procedure for preparing CPD-042 (5.9 mg, yield: 45.5%). MS (ESI) m/z=1069.1 [M+H]⁺.

### Example 150: 6-(2-(9-(2-(((2S,4R)-1-((S)-2-(1-Fluorocyclopropane-1-carboxamido)-3,3-dimethylbutanoyl)-4-hydroxypyrrolidine-2-carboxamido)methyl)-5-(4-methylthiazol-5-yl)phenoxy)nonanamido)acetamido)-4-((2-methoxy-3-(1-methyl-1H-1,2,4-triazol-3-yl)phenyl)amino)-N-methylpyridazine-3-carboxamide (CPD-108)

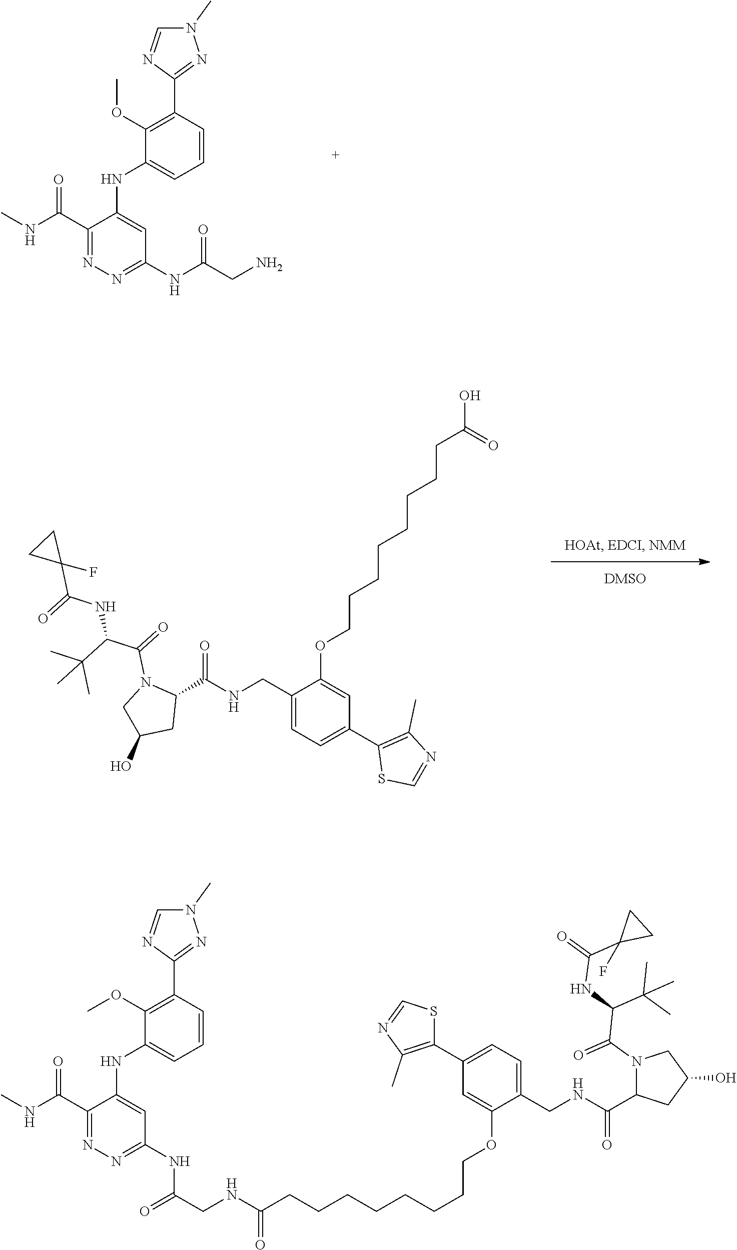 [SMILES: CNC(=O)c1nnc(NC(=O)CN)cc1Nc1cccc(-c2ncn(C)n2)c1OC ; Cc1ncsc1-c1ccc(CNC(=O)[C@@H]2C[C@@H](O)CN2C(=O)[C@@H](NC(=O)C2(F)CC2)C(C)(C)C)c(OCCCCCCCCC(=O)O)c1 ; CNC(=O)c1nnc(NC(=O)CNC(=O)CCCCCCCCOc2cc(-c3scnc3C)ccc2CNC(=O)C2C[C@@H](O)CN2C(=O)[C@@H](NC(=O)C2(F)CC2)C(C)(C)C)cc1Nc1cccc(-c2ncn(C)n2)c1OC]

CPD-108 was synthesized following the same procedure for preparing CPD-042 (1.15 mg, yield: 8.7%). MS (ESI) m/z=1083.2 [M+H]⁺.

### Example 151: 6-(2-(10-(2-(((2S,4R)-1-((S)-2-(1-Fluorocyclopropane-1-carboxamido)-3,3-dimethylbutanoyl)-4-hydroxypyrrolidine-2-carboxamido)methyl)-5-(4-methylthiazol-5-yl)phenoxy)decanamido)acetamido)-4-((2-methoxy-3-(1-methyl-JH-1,2,4-triazol-3-yl)phenyl)amino)-N-methylpyridazine-3-carboxamide (CPD-109)

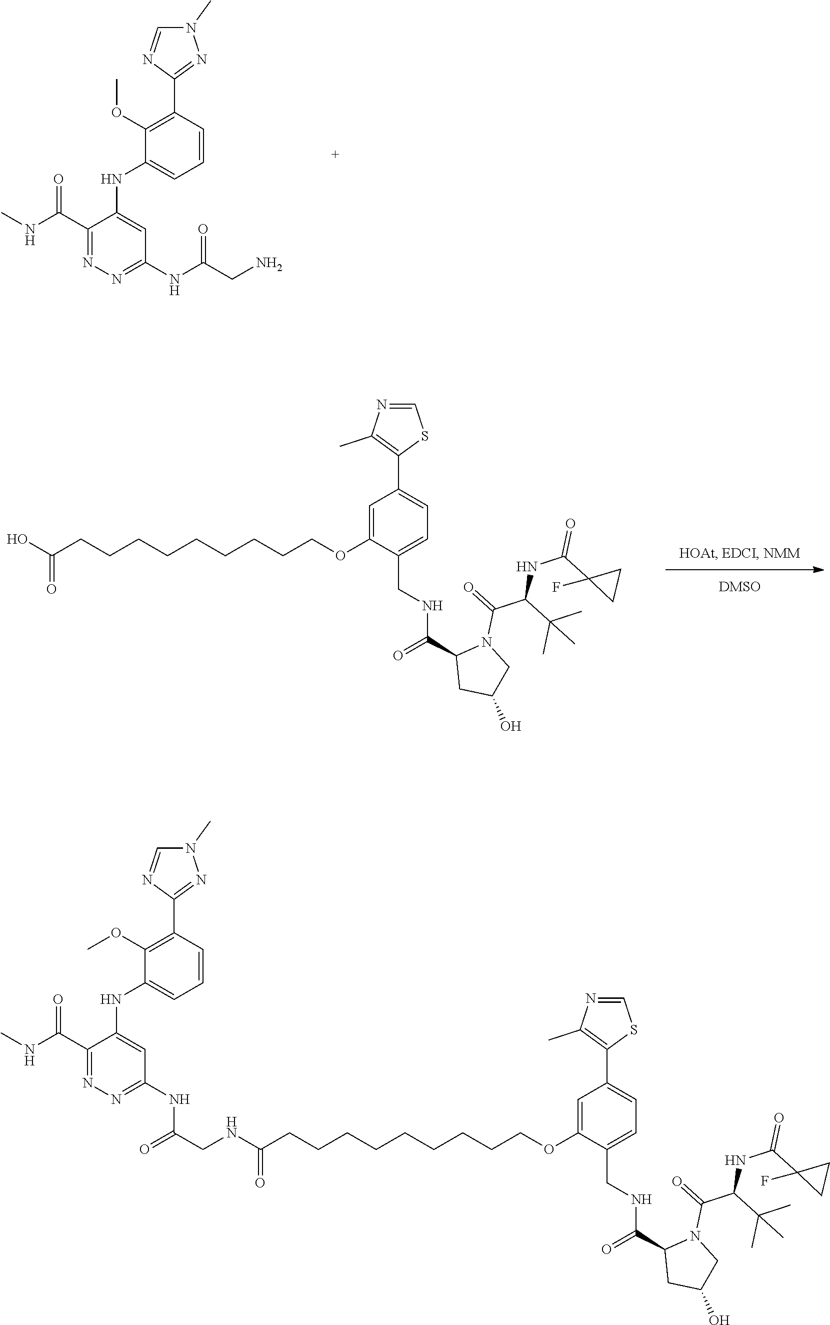 [SMILES: CNC(=O)c1nnc(NC(=O)CN)cc1Nc1cccc(-c2ncn(C)n2)c1OC ; Cc1ncsc1-c1ccc(CNC(=O)[C@@H]2C[C@@H](O)CN2C(=O)[C@@H](NC(=O)C2(F)CC2)C(C)(C)C)c(OCCCCCCCCCC(=O)O)c1 ; CNC(=O)c1nnc(NC(=O)CNC(=O)CCCCCCCCCOc2cc(-c3scnc3C)ccc2CNC(=O)[C@@H]2C[C@@H](O)CN2C(=O)[C@@H](NC(=O)C2(F)CC2)C(C)(C)C)cc1Nc1cccc(-c2ncn(C)n2)c1OC]

CPD-109 was synthesized following the same procedure for preparing CPD-042 (1.83 mg, yield: 13.7%). MS (ESI) m/z=1097.2 [M+H]⁺.

### Example 152: 6-(2-(11-(2-(((2S,4R)-1-((S)-2-(1-Fluorocyclopropane-1-carboxamido)-3,3-dimethylbutanoyl)-4-hydroxypyrrolidine-2-carboxamido)methyl)-5-(4-methylthiazol-5-yl)phenoxy)undecanamido)acetamido)-4-((2-methoxy-3-(1-methyl-1H-1,2,4-triazol-3-yl)phenyl)amino)-N-methylpyridazine-3-carboxamide (CPD-110)

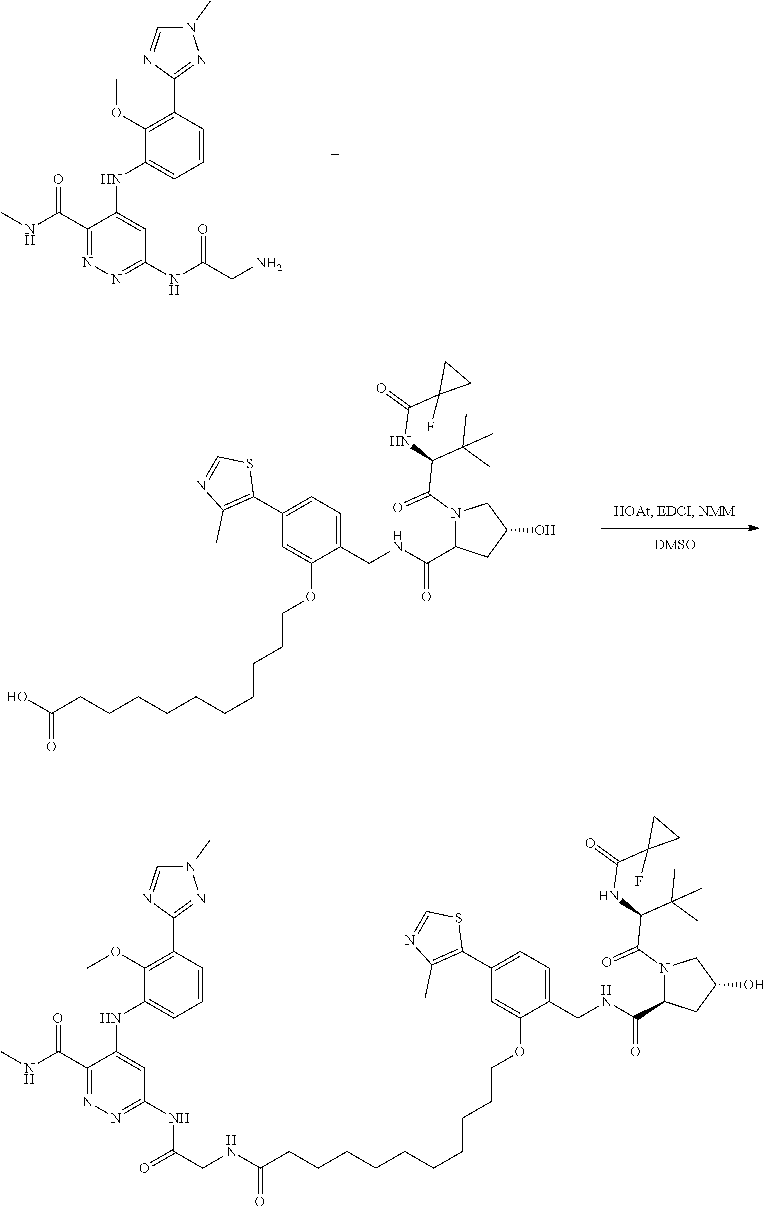 [SMILES: CNC(=O)c1nnc(NC(=O)CN)cc1Nc1cccc(-c2ncn(C)n2)c1OC ; Cc1ncsc1-c1ccc(CNC(=O)C2C[C@@H](O)CN2C(=O)[C@@H](NC(=O)C2(F)CC2)C(C)(C)C)c(OCCCCCCCCCCC(=O)O)c1 ; CNC(=O)c1nnc(NC(=O)CNC(=O)CCCCCCCCCCOc2cc(-c3scnc3C)ccc2CNC(=O)[C@@H]2C[C@@H](O)CN2C(=O)[C@@H](NC(=O)C2(F)CC2)C(C)(C)C)cc1Nc1cccc(-c2ncn(C)n2)c1OC]

CPD-110 was synthesized following the same procedure for preparing CPD-042 (2.22 mg, yield: 16.5%). MS (ESI) m/z=1111.1 [M+H]⁺.

### Example 153: ((S)-3-((2S,4R)-1-((S)-2-(1-Fluorocyclopropane-1-carboxamido)-3,3-dimethylbutanoyl)-4-hydroxypyrrolidine-2-carboxamido)-3-(4-(4-methylthiazol-5-yl)phenyl)propanoyl)glycine (Handle 42)

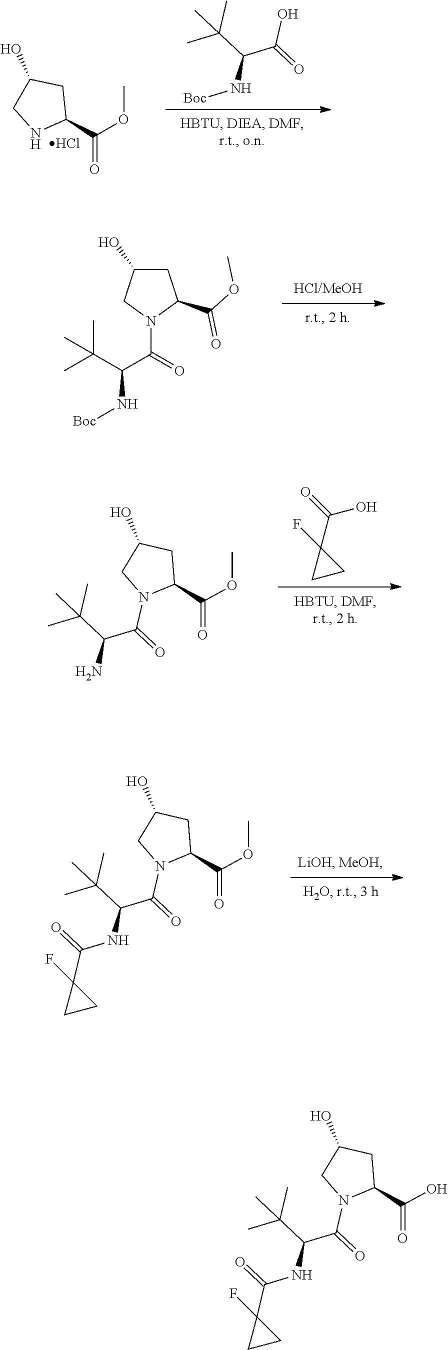

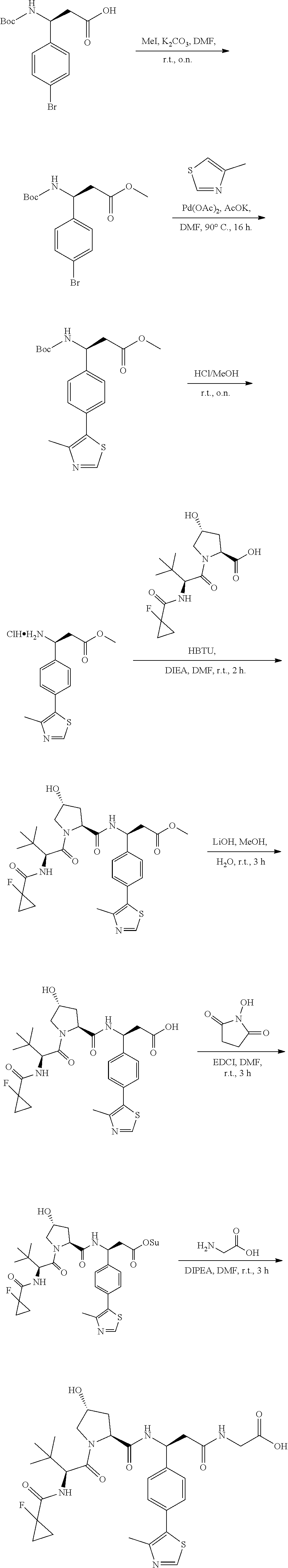

### Step 1. Synthesis of methyl (2S,4R)-1-((S)-2-((tert-butoxycarbonyl)amino)-3,3-dimethylbutanoyl)-4-hydroxypyrrolidine-2-carboxylate

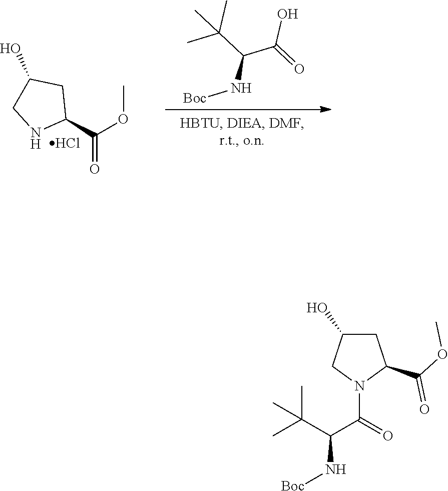

To a solution of (S)-2-((tert-butoxycarbonyl)amino)-3,3-dimethylbutanoic acid (24.0 g, 104 mmol), HBTU (59.4 g, 157 mmol) and DIEA (40.4 g, 313 mmol) in DMF (285 mL) was added methyl (2S,4R)-4-hydroxypyrrolidine-2-carboxylate hydrochloride (19.0 g, 104 mmol) at 0° C. After the mixture was stirred at rt overnight, it was diluted with water (1 L) and extracted with EtOAc (500 mL×3). The combined organic phase was washed with HCl (300 mL, 1 N), aq. K₂CO₃ (300 mL, 1N) and brine (500 mL×2), dried over Na₂SO₄, filtered, and concentrated under reduced pressure. The residue was purified by silica gel column chromatography (petroleum ether/EtOAc=1:1) to give the title compound (38.0 g, 100% yield) as a yellow oil. MS (ESI) m/z=359.2 [M+H]⁺.

### Step 2. Synthesis of methyl (2S,4R)-1-((S)-2-amino-3,3-dimethylbutanoyl)-4-hydroxypyrrolidine-2-carboxylate

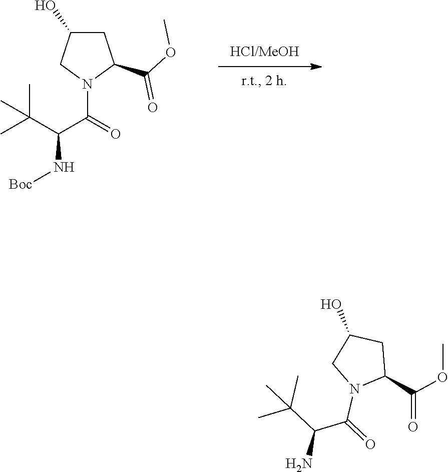 [SMILES: COC(=O)[C@@H]1C[C@@H](O)CN1C(=O)[C@@H](NC(=O)OC(C)(C)C)C(C)(C)C ; COC(=O)[C@@H]1C[C@@H](O)CN1C(=O)[C@@H](N)C(C)(C)C]

A solution of methyl (2S,4R)-1-((S)-2-((tert-butoxycarbonyl)amino)-3, 3-dimethylbutanoyl)-4-hydroxypyrrolidine-2-carboxylate (38.0 g, 106 mmol) in MeOH (200 mL) was added HCl/MeOH (200 mL, 3M) dropwise at rt. The resulting mixture was stirred at rt for 2 h, before it was concentrated under reduced pressure. The residue was washed with Et₂O (200 mL) to give the title compound (20.0 g, 67.7% yield) as a white solid. MS (ESI) m/z=259.1 [M+H]⁺.

### Step 3. Synthesis of methyl (2S,4R)-1-((S)-2-(1-fluorocyclopropane-1-carboxamido)-3,3-dimethylbutanoyl)-4-hydroxypyrrolidine-2-carboxylate

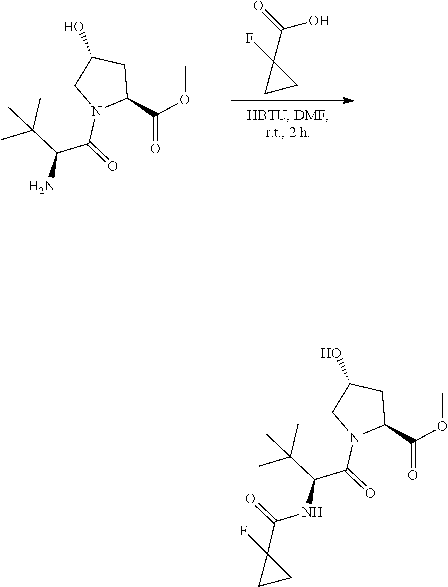 [SMILES: COC(=O)[C@@H]1C[C@@H](O)CN1C(=O)[C@@H](N)C(C)(C)C ; O=C(O)C1(F)CC1 ; COC(=O)[C@@H]1C[C@@H](O)CN1C(=O)[C@@H](NC(=O)C1(F)CC1)C(C)(C)C]

To a solution of 1-fluorocyclopropane-1-carboxylic acid (4.10 g, 39.4 mmol), HBTU (18.5 g, 48.8 mmol) and DIEA (25.8 g, 200 mmol) in DMF (160 mL) was added methyl (2S,4R)-1-((S)-2-amino-3,3-dimethylbutanoyl)-4-hydroxypyrrolidine-2-carboxylate hydrochloride (16.5 g, 55.9 mmol) at 0° C. After the mixture was stirred at rt for 2 h, it was diluted with water (500 mL) and extracted with EtOAc (300 mL×3). The combined organic phase was washed with HCl (200 mL, 1 N), aq. K₂CO₃ (200 mL, 1N) and brine (200 mL×2), dried over Na₂SO₄, filtered, and concentrated under reduced pressure. The residue was purified by silica gel column chromatography (petroleum ether/EtOAc=1:1) to give the title compound (15.0 g, 83.3% yield) as a yellow oil. MS (ESI) m/z=345.1 [M+H]⁺.

### Step 4. Synthesis of (2S,4R)-1-((S)-2-(1-fluorocyclopropane-1-carboxamido)-3,3-dimethylbutanoyl)-4-hydroxypyrrolidine-2-carboxylicacid

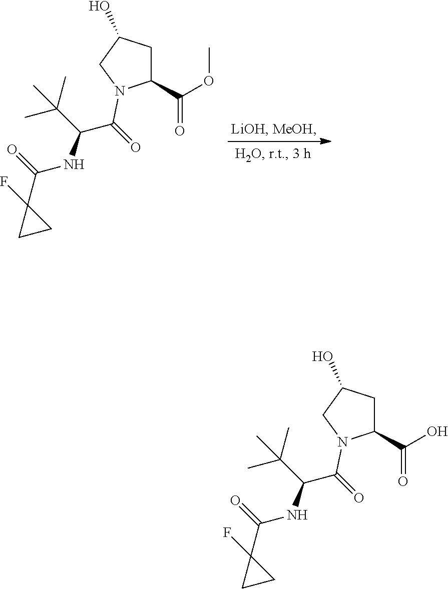 [SMILES: COC(=O)[C@@H]1C[C@@H](O)CN1C(=O)[C@@H](NC(=O)C1(F)CC1)C(C)(C)C ; CC(C)(C)[C@H](NC(=O)C1(F)CC1)C(=O)N1C[C@H](O)C[C@H]1C(=O)O]

A solution of methyl (2S,4R)-1-((S)-2-(1-fluorocyclopropane-1-carboxamido)-3, 3-dimethylbutanoyl)-4-hydroxypyrrolidine-2-carboxylate (15.0 g, 43.6 mmol) and LiOH·H₂O (3.00 g, 71.4 mmol) in MeOH (30 mL) and H₂O (30 mL) was stirred at rt for 3 h, before it was diluted with water (50 mL) and acidified pH to 2 with HCl (1N). The mixture was extracted with EtOAc (100 mL×3), washed with brine (50 mL×3), dried over Na₂SO₄, filtered, and concentrated to give the title compound (10.0 g, yield: 71.4%) as a white solid. MS (ESI) m/z=331.0 [M+H]⁺.

### Step 5. Synthesis of methyl (S)-3-(4-bromophenyl)-3-((tert-butoxycarbonyl)amino)propanoate

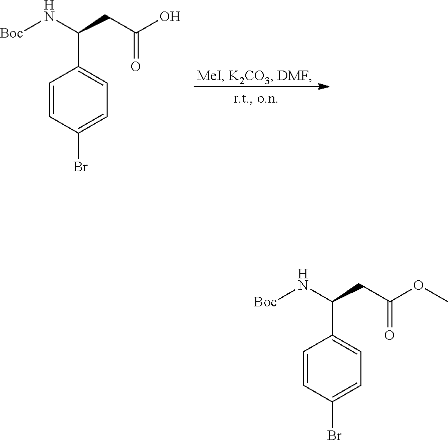 [SMILES: CC(C)(C)OC(=O)N[C@@H](CC(=O)O)c1ccc(Br)cc1 ; COC(=O)C[C@H](NC(=O)OC(C)(C)C)c1ccc(Br)cc1]

To a solution of (S)-3-(4-bromophenyl)-3-((tert-butoxycarbonyl)amino)propanoic acid (15.0 g, 43.7 mmol) and K₂CO₃ (9.00 g, 65.0 mmol) in DMF (150 mL) was added iodomethane (9.00 g, 63.0 mmol) dropwise at 0° C. After the mixture was stirred at rt overnight, it was diluted with water (400 mL) and extracted with EtOAc (200 mL×3). The combined organic phase was washed with brine (200 mL×2), dried over Na₂SO₄, filtered, and concentrated under reduced pressure. The residue was purified by silica gel column chromatography (petroleum ether/EtOAc=5:1) to give the title compound (12.0 g, 76.9% yield) as a white solid. MS (ESI) m/z=358.0 [M+H]⁺.

### Step 6. Synthesis of methyl (S)-3-((tert-butoxycarbonyl)amino)-3-(4-(4-methylthiazol-5-yl)phenyl)propanoate

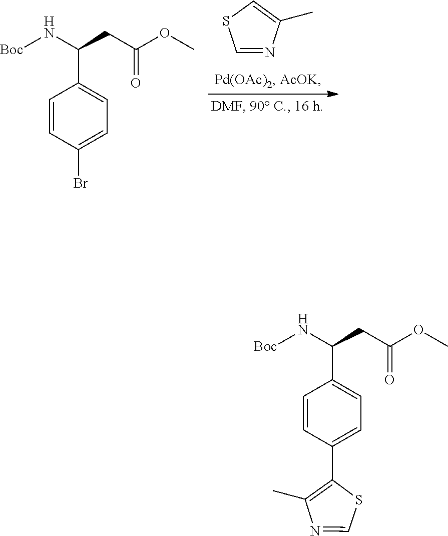

A solution of methyl (S)-3-(4-bromophenyl)-3-((tert-butoxycarbonyl)amino)propanoate (12.0 g, 33.5 mmol), Pd(OAc)₂ (750 mg, 3.35 mmol), AcOK (6.57 g, 67.0 mmol) and 4-methylthiazole (6.64 g, 67.0 mmol) in DMF (60 mL) was stirred at 90° C. for 16 h. After cooling down to rt, the mixture was diluted with water (200 mL) and extracted with EtOAc (200 mL×3). The combined organic phase was washed with brine (100 mL×2), dried over Na₂SO₄, filtered, and concentrated under reduced pressure. The residue was purified by silica gel column chromatography (petroleum ether/EtOAc=1:1) to give the title compound (12.0 g, crude) as a yellow oil. MS (ESI) m/z=377.0 [M+H]⁺.

### Step 7. Synthesis of methyl (S)-3-amino-3-(4-(4-methylthiazol-5-yl)phenyl)propanoate hydrochloride

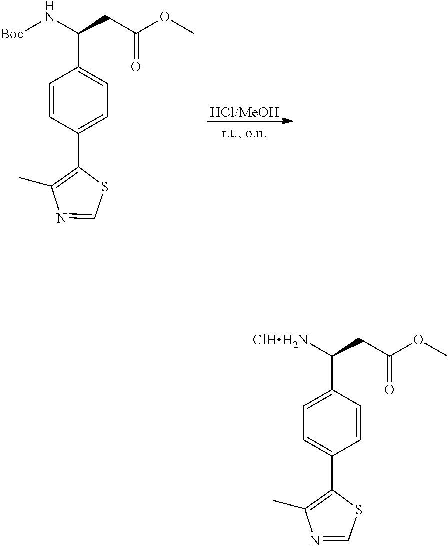

A solution of methyl (S)-3-amino-3-(4-(4-methylthiazol-5-yl)phenyl)propanoate hydrochloride (13.0 g, crude) in MeOH (200 mL) was added HCl/MeOH (200 mL, 3M) dropwise. After the mixture was stirred at rt overnight, it was concentrated in vacuum to remove MeOH. The residue was diluted with water (200 mL) and pH was adjusted to 10 with aq. K₂CO₃ (1N) and extracted with EtOAc (200 mL×3). The combined organic phase was washed with brine (100 mL×3), dried over Na₂SO₄, filtered, and concentrated under reduced pressure. The resulting residue was purified by silica gel column chromatography (petroleum ether/EtOAc=1:1 to DCM/MeOH=20:1) to give the title compound (6.20 g, 67.4% yield over two steps) as a yellow oil. MS (ESI) m/z=277.0 [M+H]⁺.

### Step 8. Synthesis of methyl (S)-3-((2S,4R)-1-((S)-2-(1-fluorocyclopropane-1-carboxamido)-3,3-dimethylbutanoyl)-4-hydroxypyrrolidine-2-carboxamido)-3-(4-(4-methylthiazol-5-yl)phenyl)propanoate

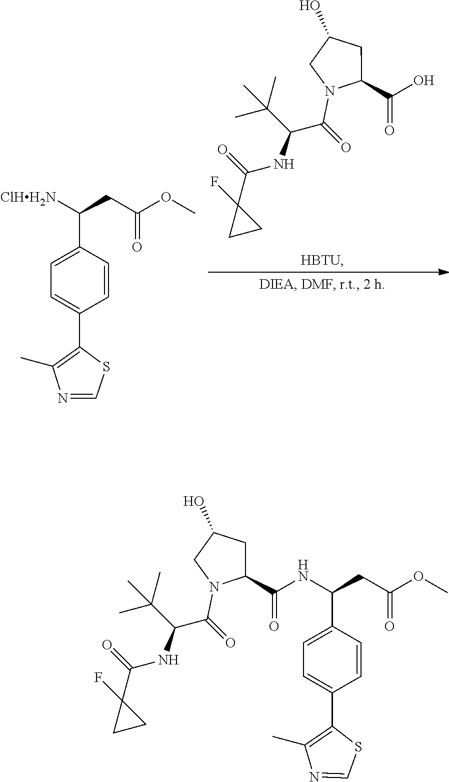

To a solution of (2S,4R)-1-((S)-2-(1-fluorocyclopropane-1-carboxamido)-3,3-dimethylbutanoyl)-4-hydroxypyrrolidine-2-carboxylic acid (5.98 g, 18.1 mmol), HBTU (9.60 g, 25.3 mmol) and DIEA (11.7 g, 90.7 mmol) in DMF (90 mL) was added methyl (S)-3-amino-3-(4-(4-methylthiazol-5-yl)phenyl)propanoate hydrochloride (6.20 g, 22.4 mmol) at 0° C. After the mixture was stirred at rt for 2 h, it was diluted with water (500 mL) and extracted with EtOAc (200 mL×3). The combined organic phase was washed with HCl (100 mL, 1 N), aq. K₂CO₃ (100 mL, 1N) and brine (200 mL×2), dried over Na₂SO₄, filtered, and concentrated under reduced pressure. The residue was purified by silica gel column chromatography (petroleum ether/EtOAc=1:1) to give the title compound (6.70 g, 63.0% yield) as a yellow oil. MS (ESI) m/z=589.3 [M+H]⁺.

### Step 9. Synthesis of (S)-3-((2S,4R)-1-((S)-2-(1-fluorocyclopropane-1-carboxamido)-3,3-dimethylbutanoyl)-4-hydroxypyrrolidine-2-carboxamido)-3-(4-(4-methylthiazol-5-yl)phenyl)propanoic acid

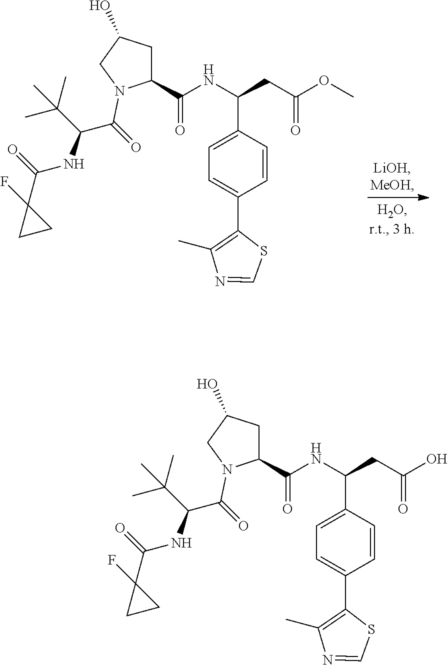 [SMILES: COC(=O)C[C@H](NC(=O)[C@@H]1C[C@@H](O)CN1C(=O)[C@@H](NC(=O)C1(F)CC1)C(C)(C)C)c1ccc(-c2scnc2C)cc1 ; Cc1ncsc1-c1ccc([C@H](CC(=O)O)NC(=O)[C@@H]2C[C@@H](O)CN2C(=O)[C@@H](NC(=O)C2(F)CC2)C(C)(C)C)cc1]

A solution of (S)-3-((2S,4R)-1-((S)-2-(1-fluorocyclopropane-1-carboxamido)-3, 3-dimethylbutanoyl)-4-hydroxypyrrolidine-2-carboxamido)-3-(4-(4-methylthiazol-5-yl)phenyl)propanoate (6.0 g, 102 mmol) and LiOH·H₂O (3.43 g, 816 mmol) in MeOH (40 mL) and H₂O (40 mL) was stirred at rt for 3 h, before it was diluted with water (100 mL). After the pH of the reaction was adjusted to 2 with HCl (1N), the mixture was extracted with EtOAc (100 mL×3). The combined organic phase was washed with brine (100 mL×5), dried over Na₂SO₄, filtered, and concentrated in vacuum to give the title compound (4.70 g, yield: 80.2%) as a white solid. MS (ESI) m/z=575.3 [M+H]⁺.

### Step 10. Synthesis of 2,5-dioxopyrrolidin-1-yl (S)-3-((2S,4R)-1-((S)-2-(1-fluorocyclopropane-1-carboxamido)-3,3-dimethylbutanoyl)-4-hydroxypyrrolidine-2-carboxamido)-3-(4-(4-methylthiazol-5-yl)phenyl)propanoate

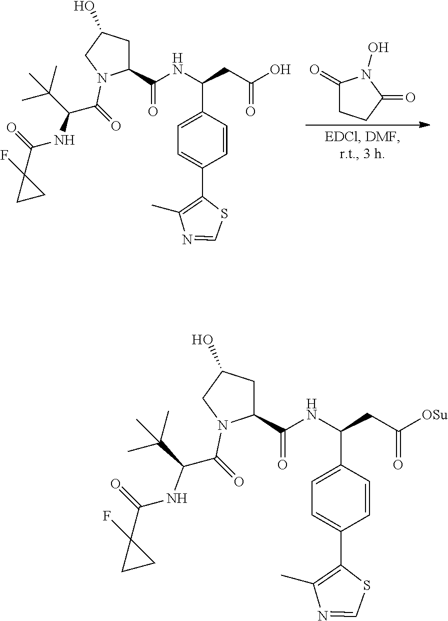

To a solution of (S)-3-((2S,4R)-1-((S)-2-(1-fluorocyclopropane-1-carboxamido)-3,3-dimethylbutanoyl)-4-hydroxypyrrolidine-2-carboxamido)-3-(4-(4-methylthiazol-5-yl)phenyl)propanoic acid (3.40 g, 5.92 mmol) in DMF (40.0 ml) were added EDCI (2.26 g, 11.9 mmol) and 1-hydroxypyrrolidine-2,5-dione (1.36 g, 11.8 mmol). After the mixture was stirred at rt for 3 h, it was diluted with H₂O (200 mL) and extracted with EtOAc (200 mL×3). The combined organic layers were washed with brine, dried over Na₂SO₄, filtered, and concentrated to give the title compound (3.10 g, crude) as a yellow solid which was used directly in the next step without further purification.

### Step 11. Synthesis of ((S)-3-((2S,4R)-1-((S)-2-(1-fluorocyclopropane-1-carboxamido)-3,3-dimethylbutanoyl)-4-hydroxypyrrolidine-2-carboxamido)-3-(4-(4-methylthiazol-5-yl)phenyl)propanoyl)glycine

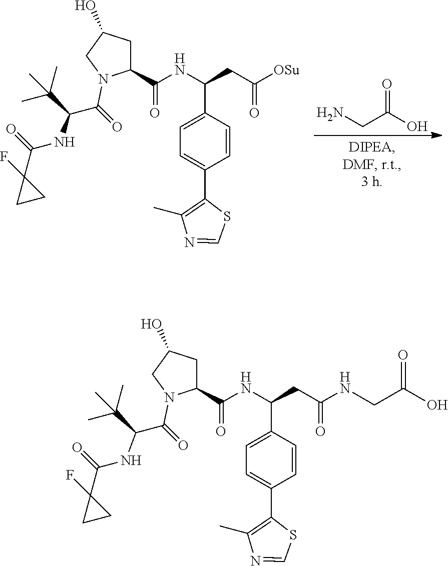

To a solution of glycine (22.4 mg, 0.298 mmol), 2,5-dioxopyrrolidin-1-yl(S)-3-((2S,4R)-1-((S)-2-(1-fluorocyclopropane-1-carboxamido)-3,3-dimethylbutanoyl)-4-hydroxypyrrolidine-2-carboxamido)-3-(4-(4-methylthiazol-5-yl)phenyl)propanoate (200 mg, 0.298 mmol) and DIEA (192 mg, 1.49 mmol) in DMF (2.00 mL) was stirred at rt for 3 h, before it was purified by prep-HPLC (0.1% TFA) to give the title compound (101 mg, 52.5% yield) as a white solid. ¹H NMR (400 MHz, DMSO-d₆) δ 8.98 (s, 1H), 8.55 (d, J=8.0 Hz, 1H), 7.79 (t, J=5.6 Hz, 1H), 7.42-7.39 (m, 2H), 7.36-7.34 (m, 2H), 7.26-7.23 (m, 1H), 5.16 (q, J=7.2 Hz, 1H), 4.57 (d, J=8.8 Hz, 1H), 4.46-4.42 (m, 1H), 4.27 (brs, 1H), 3.77-3.66 (m, 4H), 2.68-2.66 (m, 2H), 2.45 (s, 3H), 2.06-2.01 (m, 1H), 1.76-1.74 (m, 1H), 1.40-1.33 (m, 2H), 1.22-1.19 (m, 2H), 0.97 (s, 9H). MS (ESI) m/z=632.1 [M+H]⁺.

### Example 154: 3-((S)-3-((2S,4R)-1-((S)-2-(1-Fluorocyclopropane-1-carboxamido)-3,3-dimethylbutanoyl)-4-hydroxypyrrolidine-2-carboxamido)-3-(4-(4-methylthiazol-5-yl)phenyl)propanamido)propanoic acid (Handle 43)

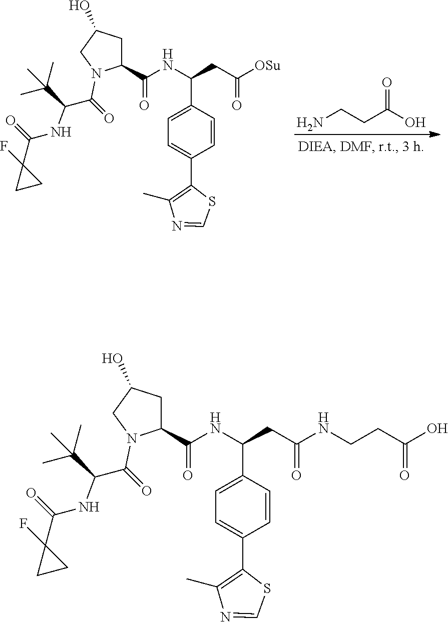

Handle 43 was synthesized following the same procedure for preparing Handle 42 (101 mg, yield: 52.5%). ¹H NMR (400 MHz, DMSO-d₆) δ 8.98 (s, 1H), 8.55 (d, J=8.0 Hz, 1H), 7.79 (t, J=5.6 Hz, 1H), 7.42-7.39 (m, 2H), 7.36-7.34 (m, 2H), 7.26-7.23 (m, 1H), 5.16 (q, J=7.2 Hz, 1H), 4.57 (d, J=8.8 Hz, 1H), 4.46-4.42 (m, 1H), 4.27 (brs, 1H), 3.62-3.54 (m, 2H), 3.15-3.13 (m, 2H), 2.58-2.56 (m, 2H), 2.49 (s, 3H), 2.13-2.10 (m, 2H), 2.06-2.01 (m, 1H), 1.76-1.74 (m, 1H), 1.40-1.33 (m, 2H), 1.22-1.19 (m, 2H), 0.97 (s, 9H). MS (ESI) m/z=646.2 [M+H]⁺.

### Example 155: 4-((S)-3-((2S,4R)-1-((S)-2-(1-Fluorocyclopropane-1-carboxamido)-3,3-dimethylbutanoyl)-4-hydroxypyrrolidine-2-carboxamido)-3-(4-(4-methylthiazol-5-yl)phenyl)propanamido)butanoic acid (Handle 44)

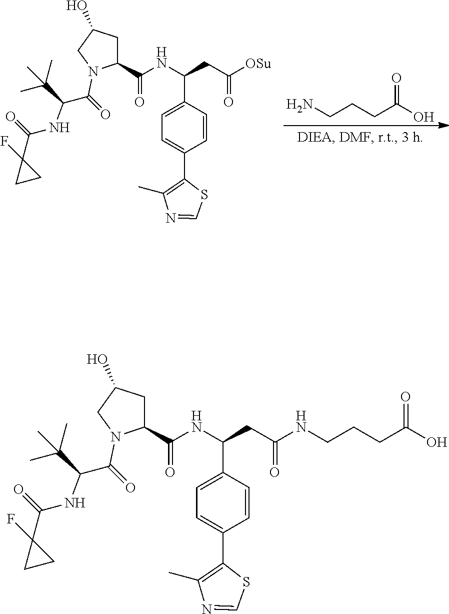

Handle 44 was synthesized following the same procedure for preparing Handle 42 (109 mg, yield: 55.6%). ¹H NMR (400 MHz, DMSO-d₆) δ 8.98 (s, 1H), 8.55 (d, J=8.0 Hz, 1H), 7.79 (t, J=5.6 Hz, 1H), 7.42-7.39 (m, 2H), 7.36-7.34 (m, 2H), 7.26-7.23 (m, 1H), 5.16 (q, J=7.2 Hz, 1H), 4.57 (d, J=8.8 Hz, 1H), 4.46-4.42 (m, 1H), 4.27 (brs, 1H), 3.59-3.54 (m, 2H), 3.01-2.94 (m, 2H), 2.58-2.56 (m, 2H), 2.49 (s, 3H), 2.13-2.10 (m, 2H), 2.06-2.01 (m, 1H), 1.76-1.74 (m, 1H), 1.40-1.33 (m, 2H), 1.22-1.19 (m, 4H), 0.97 (s, 9H). MS (ESI) m/z=660.7 [M+H]⁺.

### Example 156: 5-((S)-3-((2S,4R)-1-((S)-2-(1-Fluorocyclopropane-1-carboxamido)-3,3-dimethylbutanoyl)-4-hydroxypyrrolidine-2-carboxamido)-3-(4-(4-methylthiazol-5-yl)phenyl)propanamido)pentanoic acid (Handle 45)

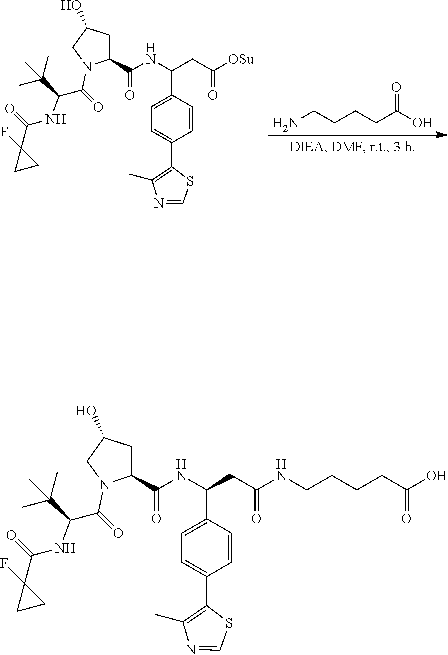

Handle 45 was synthesized following the same procedure for preparing Handle 42 (135 mg, yield: 67.5%). ¹H NMR (400 MHz, DMSO-d₆) δ 8.99 (s, 1H), 8.56 (d, J=8.0 Hz, 1H), 7.79 (t, J=5.6 Hz, 1H), 7.42-7.39 (m, 2H), 7.36-7.34 (m, 2H), 7.26-7.23 (m, 1H), 5.16 (q, J=7.2 Hz, 1H), 4.57 (d, J=8.8 Hz, 1H), 4.46-4.42 (m, 1H), 4.27 (brs, 1H), 3.62-3.54 (m, 2H), 3.00-2.97 (m, 2H), 2.58-2.56 (m, 2H), 2.49 (s, 3H), 2.13-2.10 (m, 2H), 2.06-2.01 (m, 1H), 1.76-1.74 (m, 1H), 1.42-1.33 (m, 4H), 1.27-1.14 (m, 4H), 0.97 (s, 9H). MS (ESI) m/z=674.2 [M+H]⁺.

### Example 157: 6-((S)-3-((2S,4R)-1-((S)-2-(1-Fluorocyclopropane-1-carboxamido)-3,3-dimethylbutanoyl)-4-hydroxypyrrolidine-2-carboxamido)-3-(4-(4-methylthiazol-5-yl)phenyl)propanamido)hexanoic acid (Handle 46)

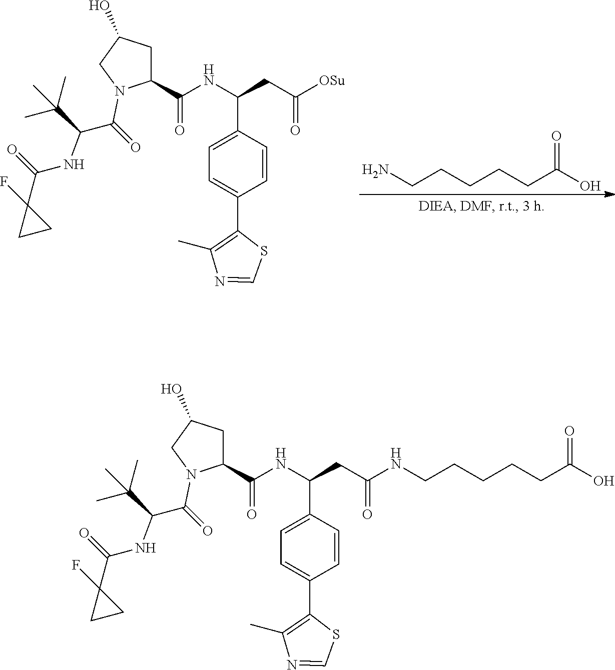

Handle 46 was synthesized following the same procedure for preparing Handle 42 (105 mg, yield: 51.2%). ¹H NMR (400 MHz, DMSO-d₆) δ 8.98 (s, 1H), 8.55 (d, J=8.0 Hz, 1H), 7.79 (t, J=5.6 Hz, 1H), 7.42-7.39 (m, 2H), 7.36-7.34 (m, 2H), 7.26-7.23 (m, 1H), 5.16 (q, J=7.2 Hz, 1H), 4.57 (d, J=8.8 Hz, 1H), 4.46-4.42 (m, 1H), 4.27 (brs, 1H), 3.62-3.54 (m, 2H), 3.10-2.97 (m, 2H), 2.58-2.56 (m, 2H), 2.49 (s, 3H), 2.13-2.10 (m, 2H), 2.06-2.01 (m, 1H), 1.76-1.74 (m, 1H), 1.40-1.33 (m, 4H), 1.22-1.19 (m, 6H), 0.97 (s, 9H). MS (ESI) m/z=688.2 [M+H]⁺.

### Example 158: 7-((S)-3-((2S,4R)-1-((S)-2-(1-Fluorocyclopropane-1-carboxamido)-3,3-dimethylbutanoyl)-4-hydroxypyrrolidine-2-carboxamido)-3-(4-(4-methylthiazol-5-yl)phenyl)propanamido)heptanoic acid (Handle 47)

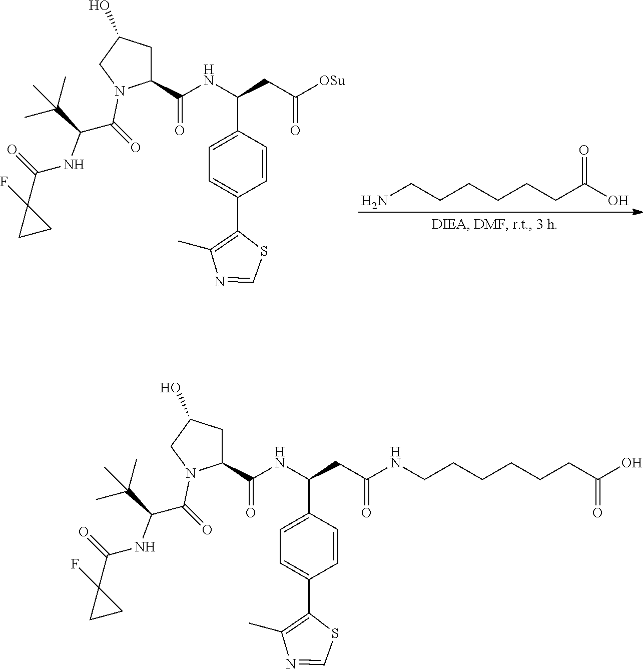

Handle 47 was synthesized following the same procedure for preparing Handle 42 (135 mg, yield: 64.9%). ¹H NMR (400 MHz, DMSO-d₆) δ 8.98 (s, 1H), 8.55 (d, J=8.0 Hz, 1H), 7.79 (t, J=5.6 Hz, 1H), 7.42-7.39 (m, 2H), 7.36-7.34 (m, 2H), 7.26-7.23 (m, 1H), 5.16 (q, J=7.2 Hz, 1H), 4.57 (d, J=8.8 Hz, 1H), 4.46-4.42 (m, 1H), 4.27 (brs, 1H), 3.62-3.54 (m, 2H), 3.15-3.13 (m, 1H), 3.00-2.97 (m, 1H), 2.58-2.56 (m, 2H), 2.49 (s, 3H), 2.13-2.10 (m, 2H), 2.06-2.01 (m, 1H), 1.76-1.74 (m, 1H), 1.40-1.33 (m, 4H), 1.22-1.19 (m, 8H), 0.97 (s, 9H). MS (ESI) m/z=702.2 [M+H]⁺.

### Example 159: 8-((S)-3-((2S,4R)-1-((S)-2-(1-Fluorocyclopropane-1-carboxamido)-3,3-dimethylbutanoyl)-4-hydroxypyrrolidine-2-carboxamido)-3-(4-(4-methylthiazol-5-yl)phenyl)propanamido)octanoic acid (Handle 48)

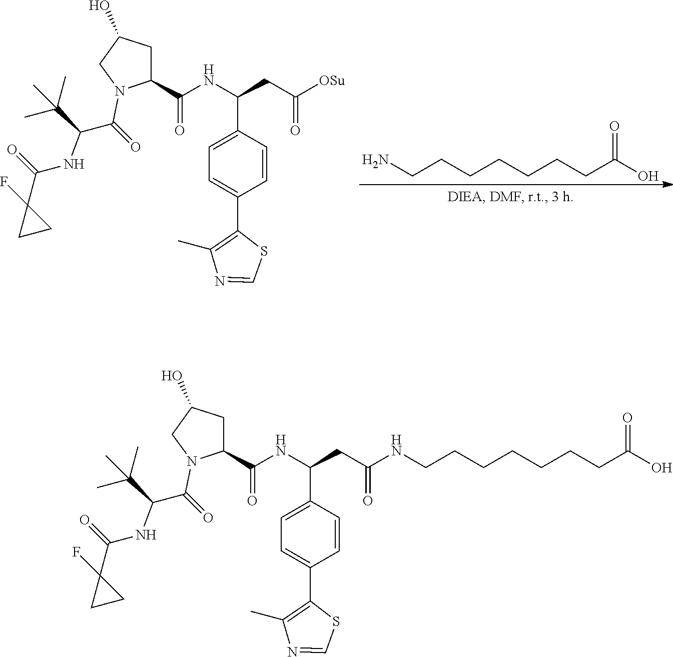

Handle 48 was synthesized following the same procedure for preparing Handle 42 (97.2 mg, yield: 45.4%). ¹H NMR (400 MHz, DMSO-d₆) δ 8.98 (s, 1H), 8.55 (d, J=8.0 Hz, 1H), 7.79 (t, J=5.6 Hz, 1H), 7.42-7.39 (m, 2H), 7.36-7.34 (m, 2H), 7.26-7.23 (m, 1H), 5.16 (q, J=7.2 Hz, 1H), 4.57 (d, J=8.8 Hz, 1H), 4.46-4.42 (m, 1H), 4.27 (brs, 1H), 3.62-3.54 (m, 2H), 3.15-3.13 (m, 1H), 3.00-2.97 (m, 1H), 2.58-2.56 (m, 2H), 2.49 (s, 3H), 2.13-2.10 (m, 2H), 2.06-2.01 (m, 1H), 1.76-1.74 (m, 1H), 1.40-1.33 (m, 4H), 1.22-1.19 (m, 10H), 0.97 (s, 9H). MS (ESI) m/z=716.2 [M+H]⁺.

### Example 160: 9-((S)-3-((2S,4R)-1-((S)-2-(1-Fluorocyclopropane-1-carboxamido)-3,3-dimethylbutanoyl)-4-hydroxypyrrolidine-2-carboxamido)-3-(4-(4-methylthiazol-5-yl)phenyl)propanamido)nonanoic acid (Handle 49)

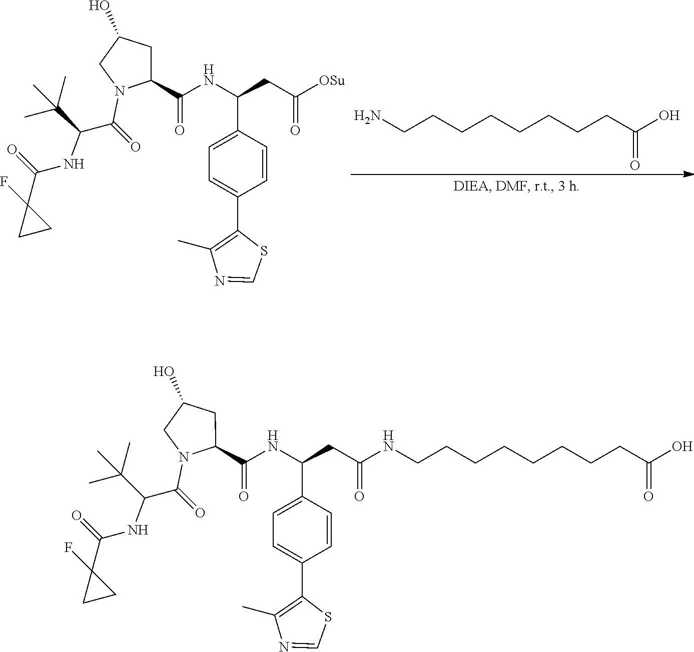

Handle 49 was synthesized following the same procedure for preparing Handle 42 (109 mg, yield: 50.2%). ¹H NMR (400 MHz, DMSO-d₆) δ 8.98 (s, 1H), 8.55 (d, J=8.0 Hz, 1H), 7.79 (t, J=5.6 Hz, 1H), 7.42-7.39 (m, 2H), 7.36-7.34 (m, 2H), 7.26-7.23 (m, 1H), 5.16 (q, J=7.2 Hz, 1H), 4.57 (d, J=8.8 Hz, 1H), 4.46-4.42 (m, 1H), 4.27 (brs, 1H), 3.62-3.54 (m, 2H), 3.15-3.13 (m, 1H), 3.00-2.97 (m, 1H), 2.58-2.56 (m, 2H), 2.49 (s, 3H), 2.13-2.10 (m, 2H), 2.06-2.01 (m, 1H), 1.76-1.74 (m, 1H), 1.40-1.33 (m, 4H), 1.22-1.19 (m, 12H), 0.97 (s, 9H). MS (ESI) m/z=730.8 [M+H]⁺.

### Example 161: 10-((S)-3-((2S,4R)-1-((S)-2-(1-Fluorocyclopropane-1-carboxamido)-3,3-dimethylbutanoyl)-4-hydroxypyrrolidine-2-carboxamido)-3-(4-(4-methylthiazol-5-yl)phenyl)propanamido)decanoic acid (Handle 50)

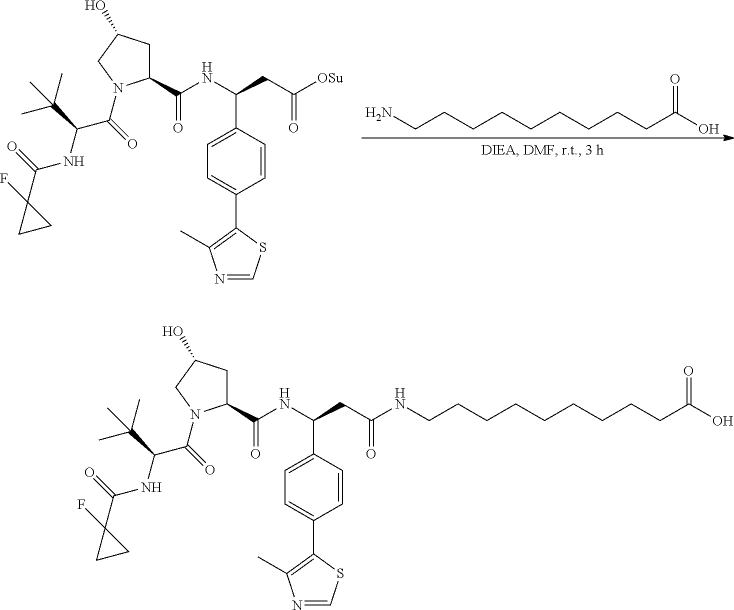

Handle 50 was synthesized following the same procedure for preparing Handle 42 (108 mg, yield: 48.8%). ¹H NMR (400 MHz, DMSO-d₆) δ 8.98 (s, 1H), 8.55 (d, J=8.0 Hz, 1H), 7.79 (t, J=5.6 Hz, 1H), 7.42-7.39 (m, 2H), 7.36-7.34 (m, 2H), 7.26-7.23 (m, 1H), 5.16 (q, J=7.2 Hz, 1H), 4.57 (d, J=8.8 Hz, 1H), 4.46-4.42 (m, 1H), 4.27 (brs, 1H), 3.62-3.54 (m, 2H), 3.15-3.13 (m, 1H), 3.00-2.97 (m, 1H), 2.58-2.56 (m, 2H), 2.49 (s, 3H), 2.13-2.10 (m, 2H), 2.06-2.01 (m, 1H), 1.76-1.74 (m, 1H), 1.40-1.33 (m, 4H), 1.22-1.19 (m, 14H), 0.97 (s, 9H). MS (ESI) m/z=744.3 [M+H]⁺.

### Example 162: 11-((S)-3-((2S,4R)-1-((S)-2-(1-Fluorocyclopropane-1-carboxamido)-3,3-dimethylbutanoyl)-4-hydroxypyrrolidine-2-carboxamido)-3-(4-(4-methylthiazol-5-yl)phenyl)propanamido)undecanoic acid (Handle 51)

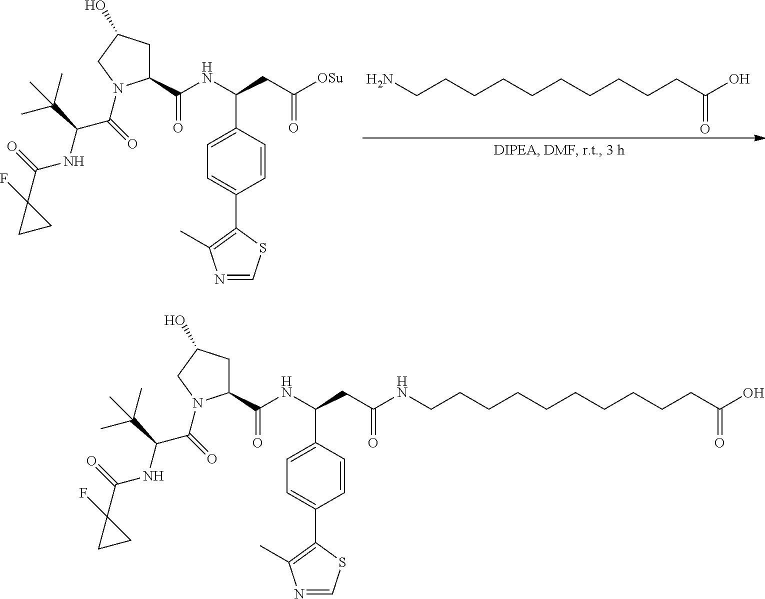

Handle 51 was synthesized following the same procedure for preparing Handle 42 (70 mg, yield: 31%). ¹H NMR (400 MHz, DMSO-d₆) δ 8.98 (s, 1H), 8.55 (d, J=8.0 Hz, 1H), 7.79 (t, J=5.6 Hz, 1H), 7.42-7.39 (m, 2H), 7.36-7.34 (m, 2H), 7.26-7.23 (m, 1H), 5.16 (q, J=7.2 Hz, 1H), 4.57 (d, J=8.8 Hz, 1H), 4.46-4.42 (m, 1H), 4.27 (brs, 1H), 3.62-3.54 (m, 2H), 3.15-3.13 (m, 1H), 3.00-2.97 (m, 1H), 2.58-2.56 (m, 2H), 2.49 (s, 3H), 2.13-2.10 (m, 2H), 2.06-2.01 (m, 1H), 1.76-1.74 (m, 1H), 1.40-1.33 (m, 4H), 1.22-1.19 (m, 16H), 0.97 (s, 9H). MS (ESI) m/z=758.3 [M+H]⁺.

### Example 163: 2-(2-(((2S,4R)-1-((S)-2-(1-Fluorocyclopropane-1-carboxamido)-3,3-dimethylbutanoyl)-4-hydroxypyrrolidine-2-carboxamido)methyl)-5-(4-methylthiazol-5-yl)phenoxy)acetic acid (Handle 52)

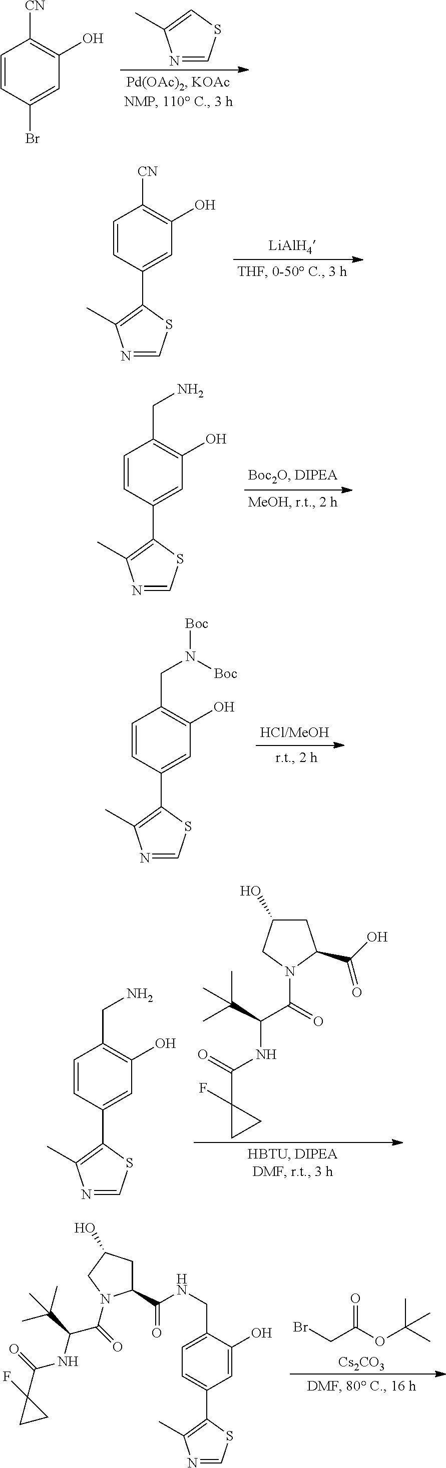

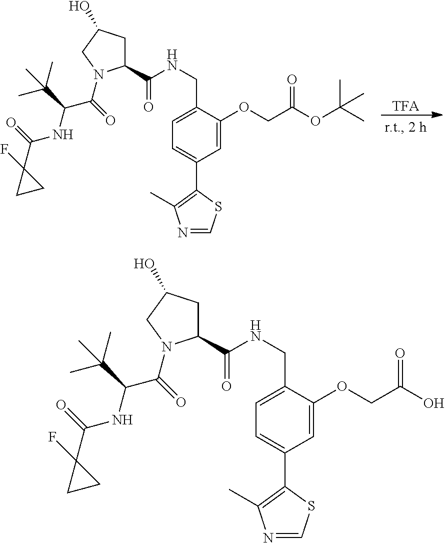 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)OC(C)(C)C)c1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)O)c1 ; "TFA"]

### Step 1. Synthesis of 2-hydroxy-4-(4-methylthiazol-5-yl)benzonitrile

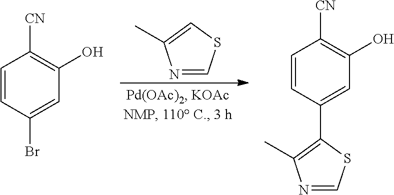

A solution of 4-bromo-2-hydroxybenzonitrile (20.0 g, 104 mmol), 4-methylthiazole (39.9 g, 208 mmol), KOAc (20.4 g, 208 mmol) and Pd(OAc)₂ (468 mg, 0.02 mmol) in AcOH (200 mL) was stirred at 110° C. for 3 h. After cooling down to rt, the mixture was diluted with H₂O (500 mL) and extracted with EtOAc (300 mL×3). The combined organic phase was washed with brine (200 mL), dried over anhydrous Na₂SO₄, filtered, and concentrated under reduced pressure. The residue was purified by silica gel column chromatography (petroleum ether/EtOAc=1:1) to give the crude product which was recrystallized with MeOH to give the title compound (15.0 g, 66.8% yield) as a yellow solid. MS (ESI) m/z=216.8 [M+H]⁺.

### Step 2. Synthesis of 2-(aminomethyl)-5-(4-methylthiazol-5-yl)phenol

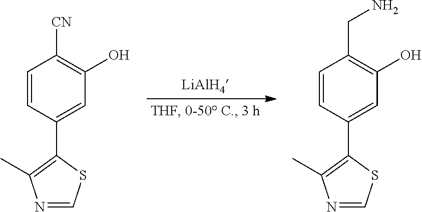

To solution of 2-hydroxy-4-(4-methylthiazol-5-yl)benzonitrile (15 g, 69.4 mmol) in dry THF (400 mL) was added LiAlH₄ (10.5 g, 278 mmol) slowly at 0° C. The mixture was stirred at 0° C. for 1 h, then it was heated to 50° C. and stirred for 3 h. After cooling down to rt, the mixture was quenched with H₂O (20 mL) and aq. NaOH (20 mL, 2M). Na₂SO₄ (200 g) was added to the mixture and stirred at rt for 1 h. The resulting residue was filtered and washed with MeOH. The filtrate was concentrated under reduced pressure to give the title compound (22.0 g, crude) as a red solid which was used directly in the next step without further purification.

### Step 3. Synthesis of N,N-diBoc 2-(aminomethyl)-5-(4-methylthiazol-5-yl)phenol

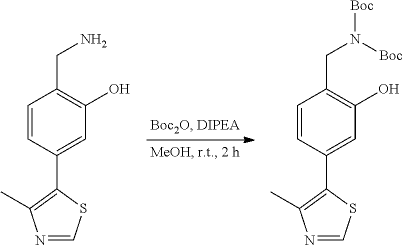 [SMILES: Cc1ncsc1-c1ccc(CN)c(O)c1 ; Cc1ncsc1-c1ccc(CN(C(=O)OC(C)(C)C)C(=O)OC(C)(C)C)c(O)c1]

To a solution of 2-(aminomethyl)-5-(4-methylthiazol-5-yl)phenol (22.0 g, crude) in MeOH (250 mL) were added DIEA (26.9 g, 208 mmol) and Boc₂O (30.1 g, 139 mmol). After the reaction mixture was stirred at rt for 3 h, it was purified by silica gel column chromatography (petroleum ether/EtOAc=1:1) to give the title compound (11.0 g, 37.7% yield) as a colorless oil. MS (ESI) m/z=421.1 [M+H]⁺.

### Step 4. Synthesis of 2-(aminomethyl)-5-(4-methylthiazol-5-yl)phenol

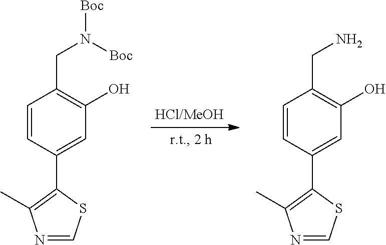 [SMILES: Cc1ncsc1-c1ccc(CN(C(=O)OC(C)(C)C)C(=O)OC(C)(C)C)c(O)c1 ; Cc1ncsc1-c1ccc(CN)c(O)c1]

A solution of the di-Boc amine (11.0 g, 26.2 mmol) in HCl/MeOH (50 mL, 3M) was stirred at rt for 2 h. The solution was concentrated under reduced pressure to give the title compound (5.5 g, HCl salt) as a white solid. MS (ESI) m/z=221.4 [M+H]⁺.

### Step 5. Synthesis of (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide

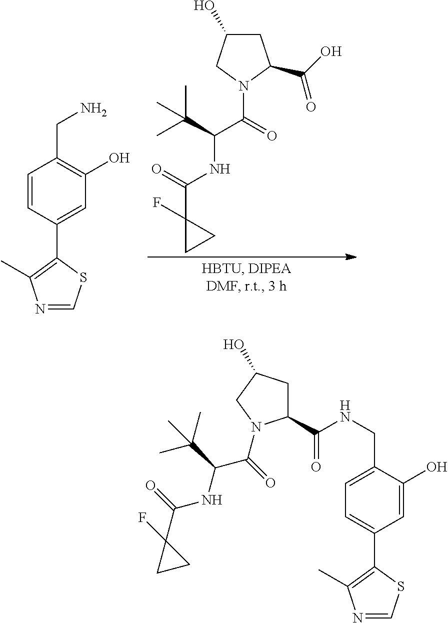

To solution of (2S,4R)-1-((S)-2-(1-fluorocyclopropane-1-carboxamido)-3,3-dimethylbutanoyl)-4-hydroxypyrrolidine-2-carboxylic acid (7.50 g, 22.7 mmol) in DMF (300 mL) were added HBTU (14.76 g, 38.8 mmol), DIEA (7.50 g, 58.2 mmol) and 2-(aminomethyl)-5-(4-methylthiazol-5-yl)phenol hydrochloride (5.00 g, HCl salt). After the mixture was stirred at rt for 3 h, it was diluted with H₂O (500 mL) and extracted with EtOAc (300 mL×3). The combined organic phase was washed with brine (200 mL), dried over anhydrous Na₂SO₄, filtered, and concentrated under reduced pressure. The residue was purified by silica gel column chromatography (petroleum ether/EtOAc=1:1) to give the title product (7.0 g, crude) as a yellow solid.

### Step 6. Synthesis of tert-butyl 2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)acetate

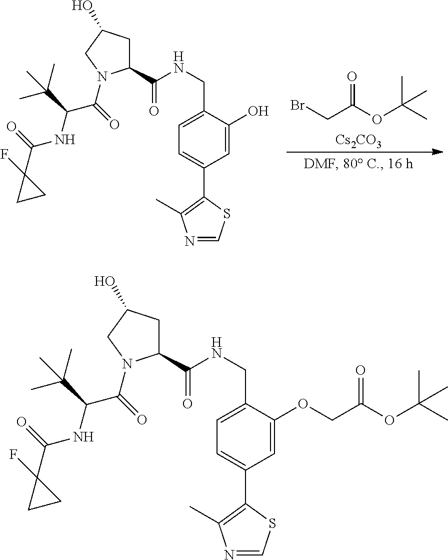

A solution of (2S,4R)-1-((S)-2-((1-(1-fluorocyclopropyl)vinyl)amino)-3,3-dimethylbutanoyl)-4-hydroxy-N-(2-hydroxy-4-(4-methylthiazol-5-yl)benzyl)pyrrolidine-2-carboxamide (200 mg, 0.377 mmol), tert-butyl 2-bromoacetate (88.2 mg, 0.453 mmol) and Cs₂CO₃ (368 mg, 1.13 mmol) in DMF (3 mL) was stirred at 80° C. for 16 h. After cooling down to rt, the mixture was purified by reverse-phase chromatography (0.1% TFA) to give the title compound (190 mg, 77.9% yield) as a white solid. MS (ESI) m/z=647.4 [M+H]⁺.

### Step 6. Synthesis of 2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)acetic acid

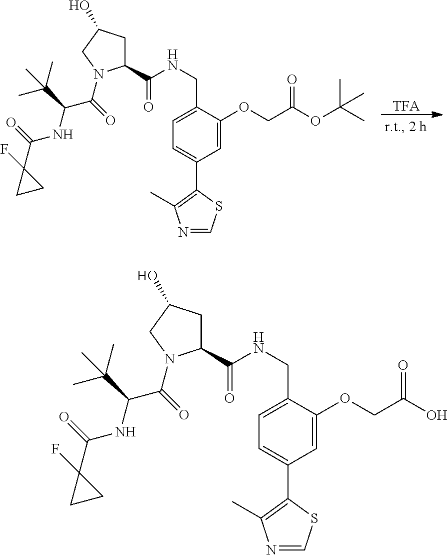 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)OC(C)(C)C)c1 ; Cc1ncsc1-c1ccc(CNC(=O)[C@@H]2C[C@@H](O)CN2C(=O)[C@@H](NC(=O)C2(F)CC2)C(C)(C)C)c(OCC(=O)O)c1 ; "TFA"]

A solution of tert-butyl 2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)acetate (190 mg, 0.294 mmol) in TFA (5 mL) was stirred at rt for 2 h. The solution was concentrated in vacuum to give the title compound (172 mg, 99.1% yield) as a white oil. ¹H NMR (400 MHz, DMSO-d₆) δ 8.98 (s, 1H), 8.54-8.51 (m, 1H), 7.42 (d, J=8.0 Hz, 1H), 7.30-7.27 (m, 1H), 6.99-6.93 (m, 2H), 4.86 (s, 2H), 4.61-4.58 (m, 1H), 4.53-4.50 (m, 1H), 4.37-4.27 (m, 3H), 3.67-3.64 (m, 2H), 2.46 (s, 3H), 2.10-2.02 (m, 1H), 1.95-1.90 (m, 1H), 1.40-1.35 (m, 2H), 1.33-1.23 (m, 2H), 0.97 (s, 9H). MS (ESI) m/z=591.6 [M+H]⁺.

### Example 164: 3-(2-(((2S,4R)-1-((S)-2-(1-Fluorocyclopropane-1-carboxamido)-3,3-dimethylbutanoyl)-4-hydroxypyrrolidine-2-carboxamido)methyl)-5-(4-methylthiazol-5-yl)phenoxy)propanoic acid (Handle 53)

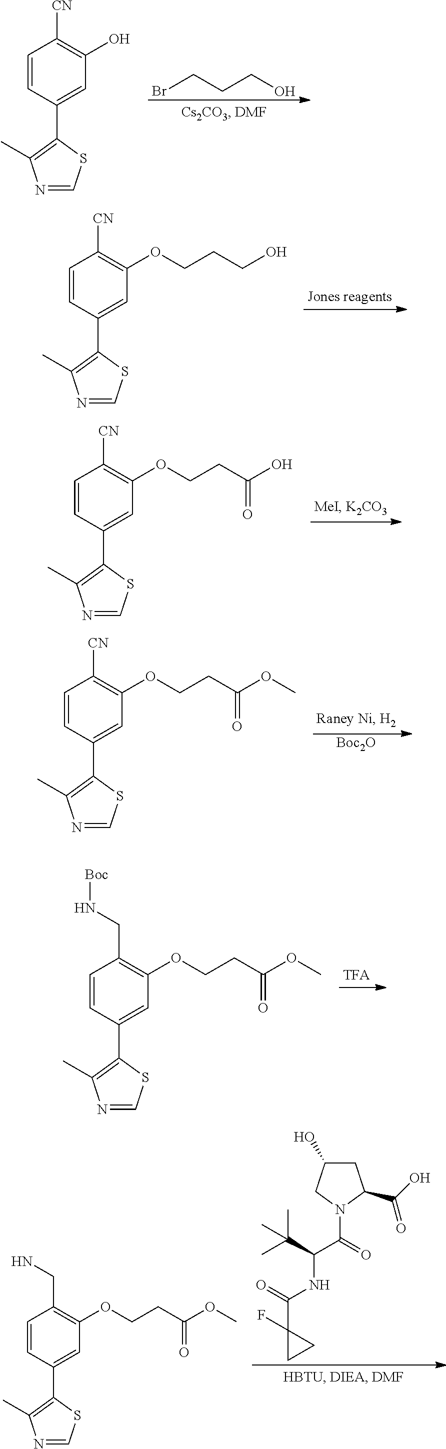

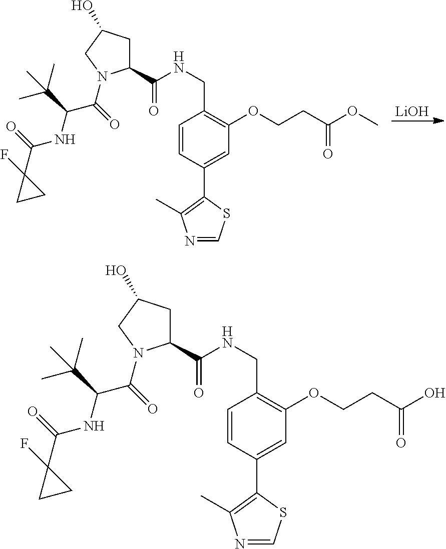

### Step 1. Synthesis of 2-(3-hydroxypropoxy)-4-(4-methylthiazol-5-yl)benzonitrile

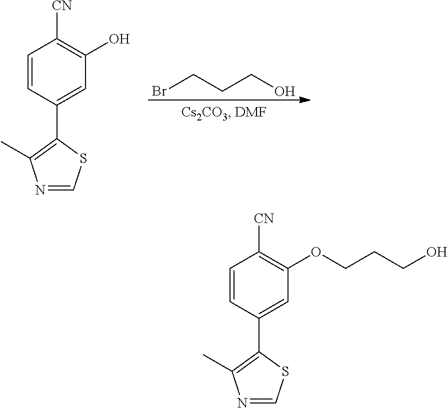 [SMILES: Cc1ncsc1-c1ccc(C#N)c(O)c1 ; OCCCBr ; Cc1ncsc1-c1ccc(C#N)c(OCCCO)c1]

To a solution of 2-hydroxy-4-(4-methylthiazol-5-yl)benzonitrile (2.20 g, 10.0 mmol) in DMF (20 mL) were added 3-bromopropan-1-ol (1.50 g, 11.0 mmol) and Cs₂CO₃ (6.50 g, 20.0 mmol) at rt under argon atmosphere. After the mixture was stirred at it for 3 h, it was diluted with H₂O (80 mL) and extracted with EtOAc (30 mL×2). The combined organic phase was washed with brine, dried over Na₂SO₄, filtered, and concentrated under reduced pressure. The residue was purified by silica gel column chromatography (petroleum ether/EtOAc=3:1) to give the title compound (2.20 g, yield: 80.3%) as a yellow solid. MS (ESI) m/z=275.0 [M+H]⁺.

### Step 2. Synthesis of 3-(2-cyano-5-(4-methylthiazol-5-yl)phenoxy)propanoic acid

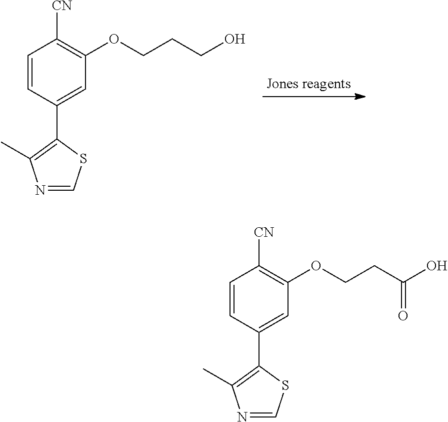 [SMILES: Cc1ncsc1-c1ccc(C#N)c(OCCCO)c1 ; Cc1ncsc1-c1ccc(C#N)c(OCCC(=O)O)c1]

To a solution of 2-(3-hydroxypropoxy)-4-(4-methylthiazol-5-yl)benzonitrile (1.90 g, 6.93 mmol) in acetone (30 mL) was added Jones reagent (4.0 mL, 10.6 mmol) dropwise at 0° C. After the mixture was stirred at rt for 2 h, it was diluted with H₂O (40 mL) and extracted with EtOAc (30 mL×2). The combined organic phase was washed with brine, dried over Na₂SO₄, filtered, and concentrated under reduced pressure. The residue was purified by silica gel column chromatography (petroleum ether/EtOAc=1:1) to give the title compound (1.80 g, yield: 90%) as a yellow solid. MS (ESI) m/z=289.0 [M+H]⁺.

### Step 3. Synthesis of methyl 3-(2-cyano-5-(4-methylthiazol-5-yl)phenoxy)propanoate

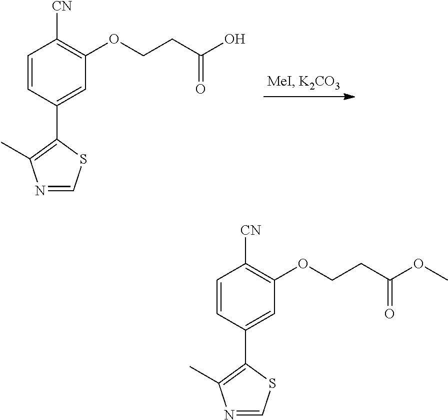

To a solution of 3-(2-cyano-5-(4-methylthiazol-5-yl)phenoxy)propanoic acid (800 mg, 2.77 mmol) in DMF (10 mL) were added K₂CO₃ (400 mg, 3.00 mmol) and Mel (440 mg, 3.00 mmol) at rt under argon atmosphere. After the mixture was stirred at rt for 2 h, it was diluted with H₂O (30 mL) and extracted with EtOAc (20 mL×3). The combined organic phase was washed with brine, dried over Na₂SO₄, filtered, and concentrated under reduced pressure. The residue was purified by silica gel column chromatography (petroleum ether/EtOAc=3:1) to give the title compound (700 mg, yield: 81.5%) as a white solid. MS (ESI) m/z=303.1 [M+H]V.

### Step 4. Synthesis of methyl 3-(2-(((tert-butoxycarbonyl)amino)methyl)-5-(4-methylthiazol-5-yl)phenoxy)propanoate

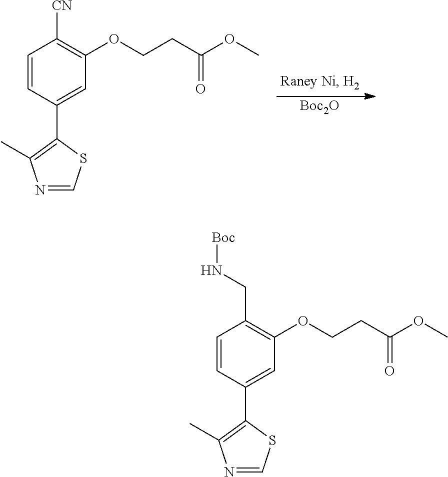

To a solution of methyl 3-(2-cyano-5-(4-methylthiazol-5-yl)phenoxy)propanoate (700 mg, 2.31 mmol) in THF (10 mL) were added Raney-Ni (70 mg) and (Boc)₂O (1.50 g, 6.90 mmol). After the mixture was stirred at rt overnight under H₂ atmosphere, it was filtered through Celite. The filtrate was concentrated under reduced pressure. The residue was purified by silica gel column chromatography (petroleum ether/EtOAc=3:1) to give the title compound (250 mg, yield: 26.1%) as a white solid. MS (ESI) m/z=407.2 [M+H]⁺.

### Step 5. Synthesis of methyl 3-(2-(aminomethyl)-5-(4-methylthiazol-5-yl)phenoxy)propanoate

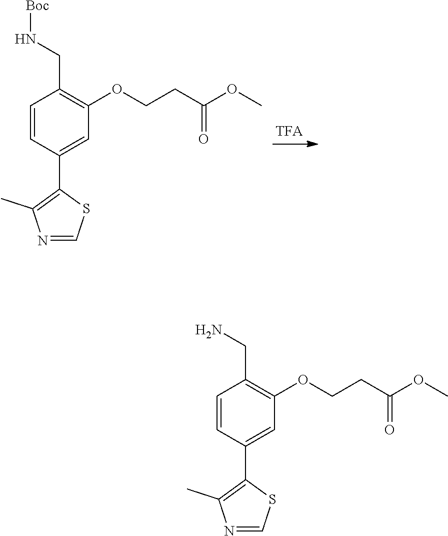

To a solution of 3-(2-(((tert-butoxycarbonyl)amino)methyl)-5-(4-methylthiazol-5-yl)phenoxy)propanoate (250 mg, 0.615 mmol) in DCM (3 mL) was added TFA (3 mL) under argon atmosphere. After the mixture was stirred at rt for 30 min, it was concentrated under reduced pressure to give the title compound (250 mg, crude) as a yellow oil which was used directly in the next step without further purification. MS (ESI) m/z=307.2 [M+H]⁺.

### Step 6. Synthesis of methyl 3-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)propanoate

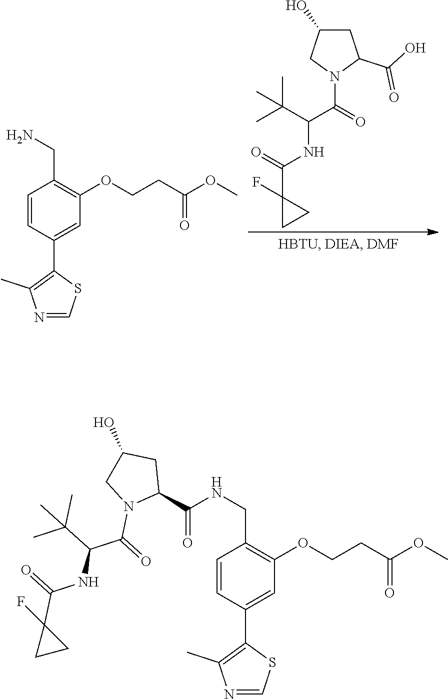

A solution of methyl 3-(2-(aminomethyl)-5-(4-methylthiazol-5-yl)phenoxy)propanoate (250 mg, 0.615 mmol), (2S,4R)-1-((S)-2-(1-fluorocyclopropane-1-carboxamido)-3,3-dimethylbutanoyl)-4-hydroxypyrrolidine-2-carboxylic acid (184 mg, 0.615 mmol), HBTU (266 mg, 0.700 mmol) and DIPEA (0.5 mL, 3.00 mmol) in DMF (3 mL) was stirred at rt for 1 h. After the reaction was diluted with H₂O (30 mL), it was extracted with EtOAc (20 mL×3). The combined organic phase was washed with brine, dried over Na₂SO₄, filtered, and concentrated under reduced pressure. The residue was purified by silica gel column chromatography (petroleum ether/EtOAc=1:2) to give the title compound (140 mg, yield: 35.7%) as a yellow oil. MS (ESI) m/z=619.4 [M+H]⁺.

### Step 7. Synthesis of 3-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)propanoic acid

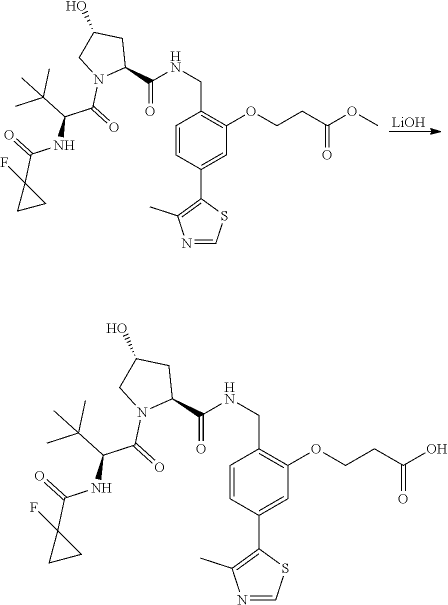

To a solution of methyl 3-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)propanoate (140 mg, 0.220 mmol) in THF (2 mL) and H₂O (2 mL) was added LiOH·H₂O (80 mg, 1.90 mmol). After the mixture was stirred at rt for 30 min, the pH was adjusted to 5 with HCl (1.0 M). The reaction was extracted with EtOAc (20 mL×3). The combined organic phase was washed with brine, dried over Na₂SO₄, filtered, and concentrated to give the title compound (63.0 mg, 47.3%) as a yellow solid. ¹H NMR (400 MHz, DMSO-d₆) δ 12.30 (brs, 1H), 8.99 (s, 1H), 8.56-7.53 (m, 1H), 7.40 (d, J=8.0 Hz, 1H), 7.29 (d, J=8.8 Hz, 1H), 7.03 (s, 1H), 6.96 (d, J=7.6 Hz, 1H), 5.17 (s, 1H), 4.61-4.58 (d, J=9.6 Hz, 1H), 4.53-4.50 (m, 1H), 4.35 (s, 1H), 4.28-4.26 (m, 3H), 4.21-4.19 (m, 1H), 3.67-3.58 (m, 2H), 2.74-2.72 (m, 2H), 2.46 (s, 3H), 2.12-2.07 (m, 1H), 1.95-1.89 (m, 1H), 1.54-1.38 (m, 2H), 1.32-1.23 (m, 2H), 1.13 (s, 9H). MS (ESI) m/z=605.0 [M+H]⁺.

### Example 165: 4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)butanoic acid (Handle 54)

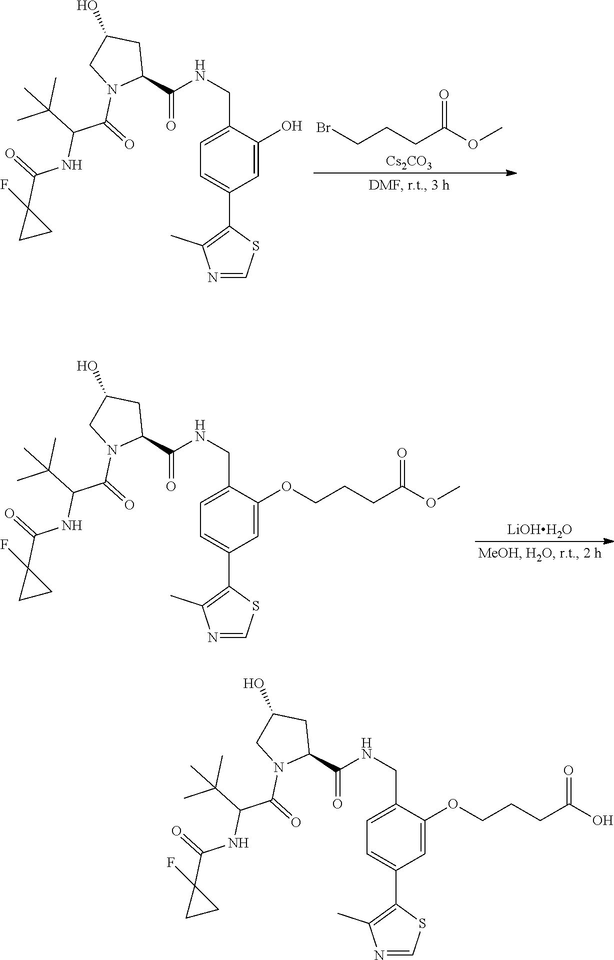

Handle 54 was synthesized following the same procedure for preparing Handle 52 (130 mg, yield: 49.9%). ¹H NMR (400 MHz, DMSO-d₆) δ 12.14 (s, 1H), 9.06 (s, 1H), 8.56-7.53 (m, 1H), 7.42 (d, J=8.0 Hz, 1H), 7.27 (d, J=11.6 Hz, 1H), 6.97-6.95 (m, 2H), 5.17 (s, 1H), 4.61-4.58 (m, 1H), 4.53-4.50 (m, 1H), 4.35 (s, 1H), 4.28-4.26 (m, 1H), 4.21-4.19 (m, 1H), 4.07-4.04 (m, 2H), 3.67-3.58 (m, 2H), 2.46 (s, 3H), 2.32-2.29 (m, 2H), 2.12-2.07 (m, 1H), 1.95-1.89 (m, 2H), 1.77-1.72 (m, 1H), 1.54-1.38 (m, 2H), 1.32-1.23 (m, 2H), 1.13 (s, 9H). MS (ESI) m/z=619.6 [M+H]⁺.

### Example 166: 5-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)pentanoic acid (Handle 55)

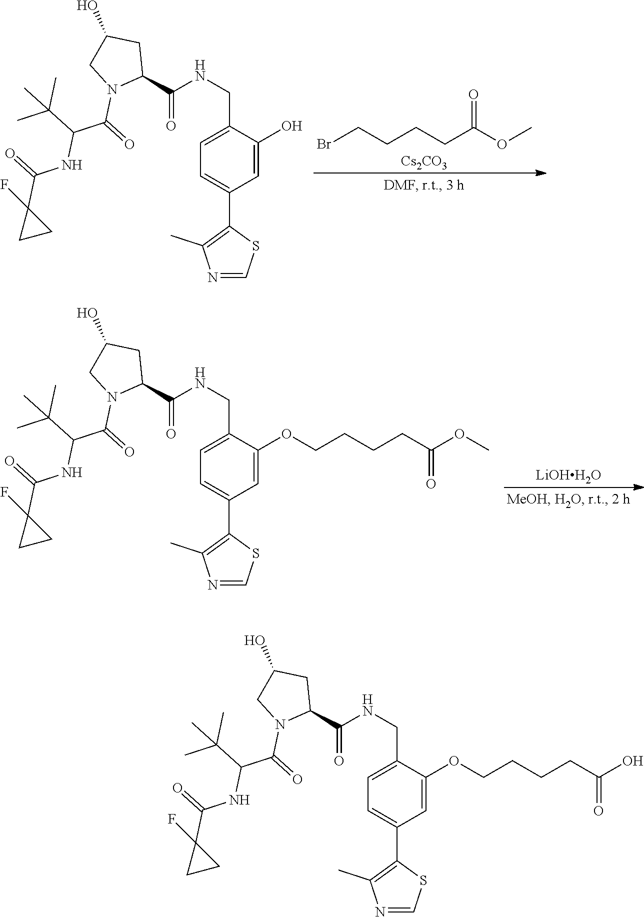

Handle 55 was synthesized following the same procedure for preparing Handle 52 (130 mg, yield: 46.1%). ¹H NMR (400 MHz, DMSO-d₆) δ 12.14 (s, 1H), 8.98 (s, 1H), 8.52-7.50 (m, 1H), 7.42 (d, J=8.0 Hz, 1H), 7.27 (d, J=11.6 Hz, 1H), 6.99-6.93 (m, 2H), 5.17 (s, 1H), 4.61-4.58 (m, 1H), 4.53-4.50 (m, 1H), 4.35 (s, 1H), 4.28-4.26 (m, 1H), 4.21-4.19 (m, 1H), 4.07-4.04 (m, 2H), 3.67-3.58 (m, 2H), 2.46 (s, 3H), 2.32-2.29 (m, 2H), 2.12-2.07 (m, 1H), 1.95-1.89 (m, 1H), 1.77-1.72 (m, 4H), 1.54-1.38 (m, 2H), 1.32-1.23 (m, 2H), 1.13 (s, 9H). MS (ESI) m/z=633.6 [M+H]⁺.

### Example 167: 6-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)hexanoic acid (Handle 56)

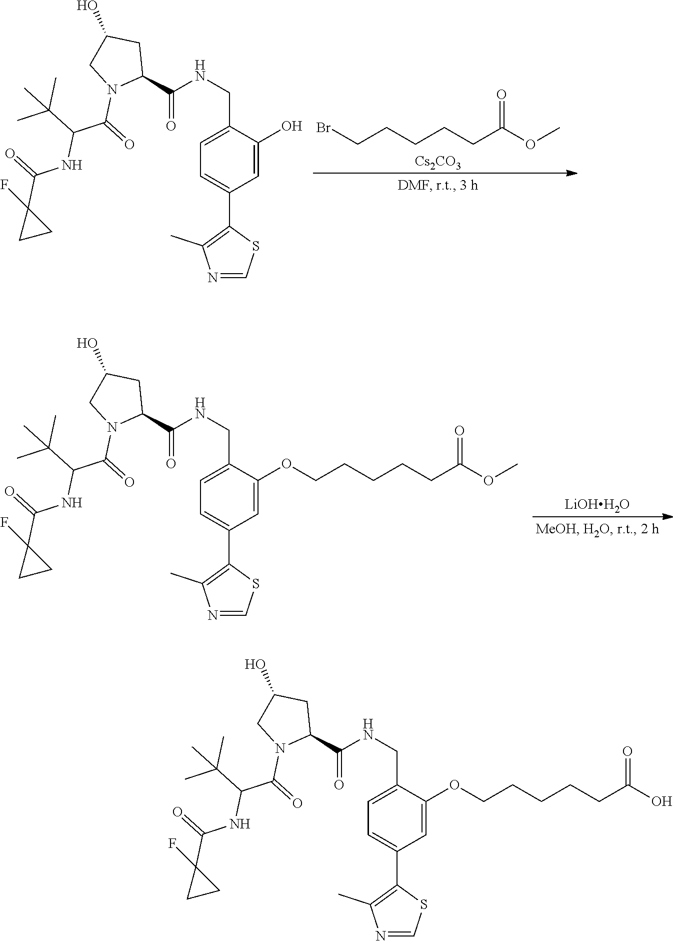

Handle 56 was synthesized following the same procedure for preparing Handle 52 (140 mg, yield: 49.6%). ¹H NMR (400 MHz, DMSO-d₆) δ 11.98 (s, 1H), 8.98 (s, 1H), 8.53-7.50 (m, 1H), 7.42 (d, J=8.0 Hz, 1H), 7.27 (d, J=11.6 Hz, 1H), 6.97-6.95 (m, 2H), 5.17 (s, 1H), 4.61-4.58 (m, 1H), 4.53-4.50 (m, 1H), 4.35 (s, 1H), 4.28-4.26 (m, 1H), 4.21-4.19 (m, 1H), 4.07-4.04 (m, 2H), 3.67-3.58 (m, 2H), 2.46 (s, 3H), 2.32-2.29 (m, 2H), 2.12-2.07 (m, 1H), 1.95-1.89 (m, 1H), 1.77-1.72 (m, 1H), 1.59-1.38 (m, 4H), 1.32-1.23 (m, 4H), 1.13 (s, 9H). MS (ESI) m/z=647.7 [M+H]⁺.

### Example 168: 7-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)heptanoic acid Handle 57)

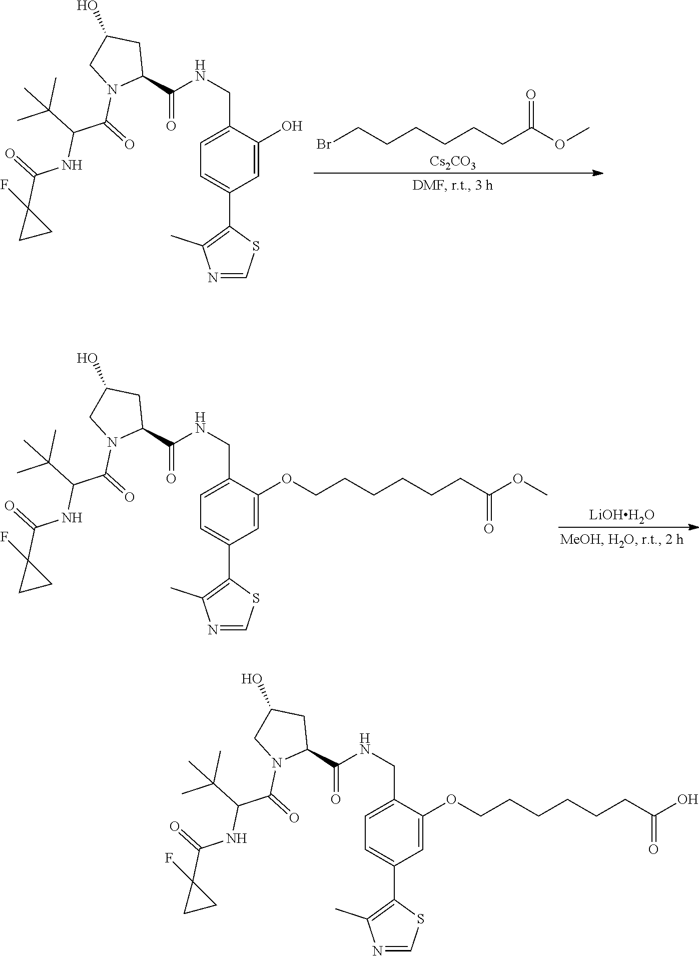

Handle 57 was synthesized following the same procedure for preparing Handle 52 (61.2 mg, yield: 20.9%). ¹H NMR (400 MHz, DMSO-d₆) δ 12.01 (s, 1H), 8.98 (s, 1H), 8.55-8.47 (m, 1H), 7.42 (d, J=8.0 Hz, 1H), 7.27 (d, J=11.6 Hz, 1H), 6.99 (s, 1H), 6.94 (d, J=7.6 Hz, 1H), 5.17 (s, 1H), 4.61-4.58 (m, 1H), 4.53-4.50 (m, 1H), 4.35 (s, 1H), 4.28-4.26 (m, 1H), 4.21-4.19 (m, 1H), 4.07-4.04 (m, 2H), 3.67-3.58 (m, 2H), 2.46 (s, 3H), 2.32-2.29 (m, 2H), 2.12-2.07 (m, 1H), 1.95-1.89 (m, 1H), 1.77-1.72 (m, 2H), 1.54-1.38 (m, 6H), 1.32-1.23 (m, 4H), 1.13 (s, 9H). MS (ESI) m/z=661.3 [M+H]⁺.

### Example 169: 8-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)octanoic acid (Handle 58)

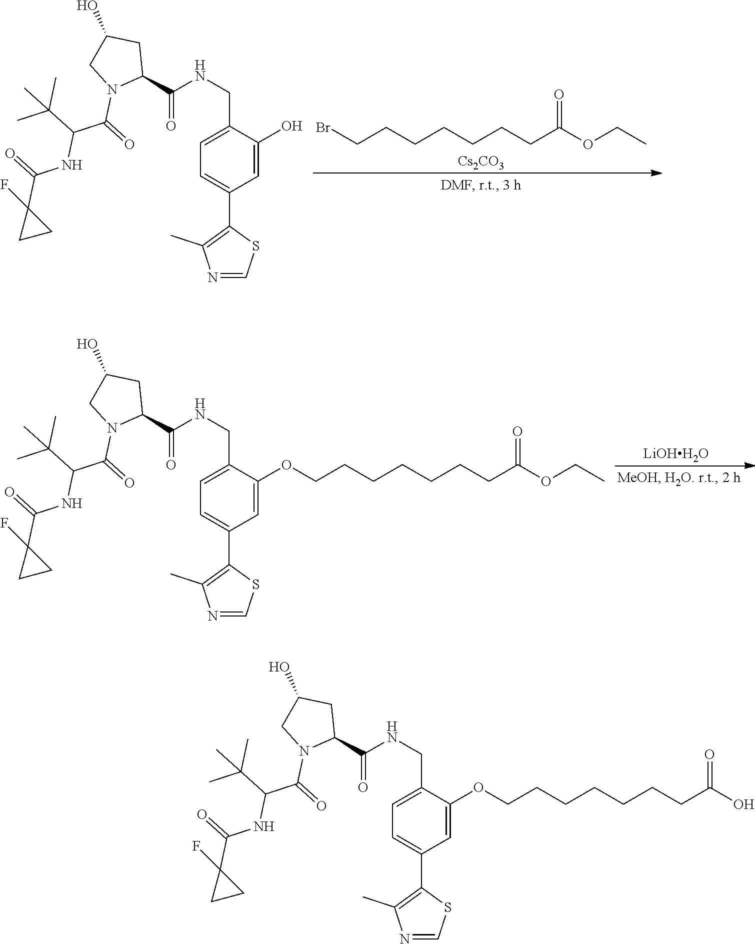

Handle 58 was synthesized following the same procedure for preparing Handle 52 (106 mg, yield: 55.8%). ¹H NMR (400 MHz, DMSO-d₆) δ 11.98 (s, 1H), 8.98 (s, 1H), 8.51-8.49 (m, 1H), 7.41 (d, J=8.0 Hz, 1H), 7.29 (d, J=11.6 Hz, 1H), 6.99 (s, 1H), 6.97-6.93 (m, 1H), 5.17 (s, 1H), 4.61-4.58 (m, 1H), 4.53-4.50 (m, 1H), 4.35-4.15 (m, 3H), 3.67-3.58 (m, 2H), 2.46 (s, 3H), 2.32-2.29 (m, 2H), 2.12-2.07 (m, 1H), 1.95-1.89 (m, 1H), 1.77-1.72 (m, 2H), 1.54-1.04 (m, 14H), 0.97 (s, 9H). MS (ESI) m/z=675.7 [M+H]⁺.

### Example 170: 9-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)nonanoic acid (Handle 59)

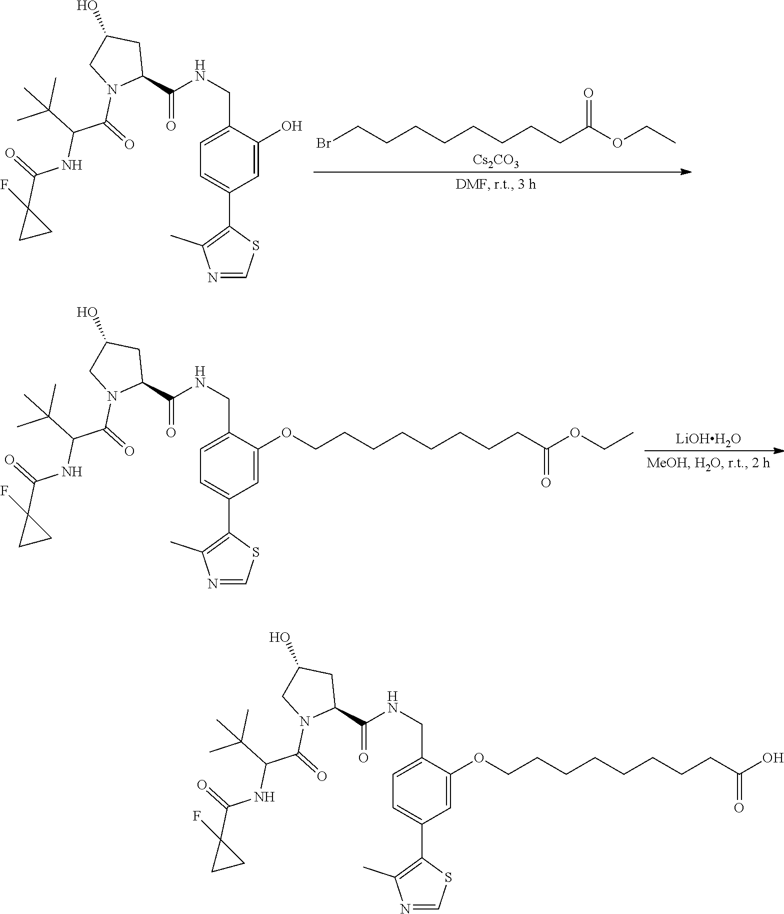

Handle 59 was synthesized following the same procedure for preparing Handle 52 (121 mg, yield: 62.3%). ¹H NMR (400 MHz, DMSO-d₆) δ 11.98 (s, 1H), 8.98 (s, 1H), 8.50-8.47 (m, 1H), 7.42 (d, J=8.0 Hz, 1H), 7.27 (d, J=11.6 Hz, 1H), 6.99 (s, 1H), 6.94 (d, J=7.6 Hz, 1H), 5.17 (s, 1H), 4.61-4.58 (m, 1H), 4.53-4.50 (m, 1H), 4.35 (s, 1H), 4.28-4.26 (m, 1H), 4.21-4.19 (m, 1H), 4.07-4.04 (m, 2H), 3.67-3.58 (m, 2H), 2.46 (s, 3H), 2.32-2.29 (m, 2H), 2.12-2.07 (m, 1H), 1.95-1.89 (m, 1H), 1.77-1.72 (m, 2H), 1.54-1.38 (m, 6H), 1.32-1.23 (m, 8H), 1.13 (s, 9H). MS (ESI) m/z=687.2 [M−H]⁻.

### Example 171: 10-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)decanoic acid (Handle 60)

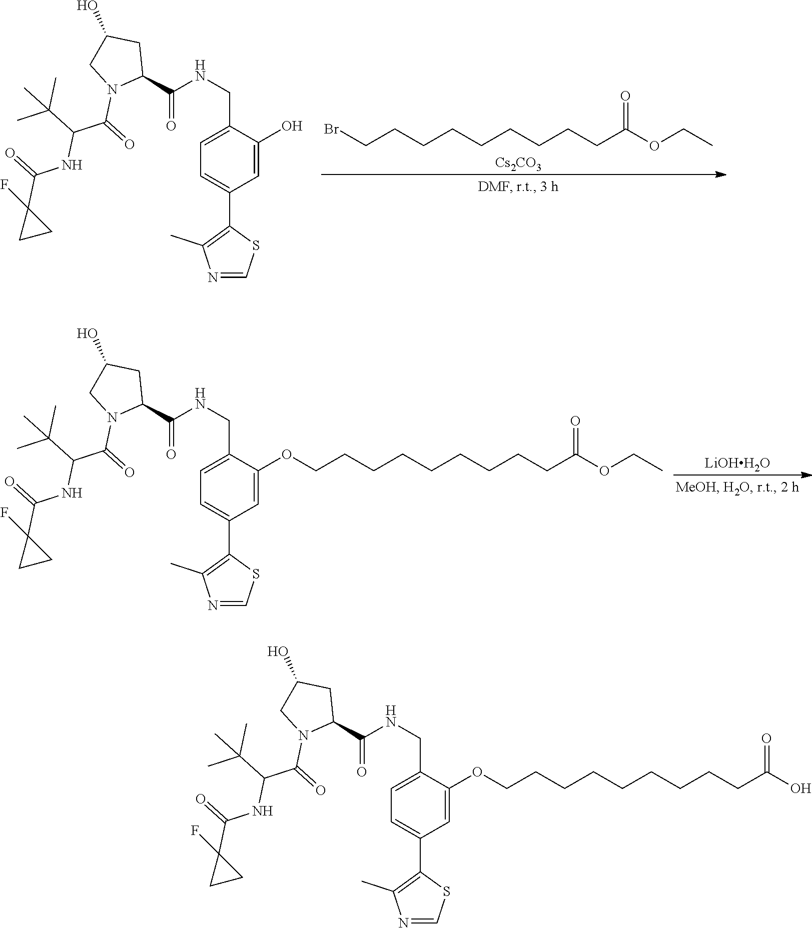

Handle 60 was synthesized following the same procedure for preparing Handle 52 (132 mg, yield: 66.6%). ¹H NMR (400 MHz, DMSO-d₆) δ 11.98 (s, 1H), 8.98 (s, 1H), 8.50-8.47 (m, 1H), 7.42 (d, J=8.0 Hz, 1H), 7.27 (d, J=11.6 Hz, 1H), 6.99 (s, 1H), 6.84 (d, J=7.6 Hz, 1H), 5.17 (s, 1H), 4.61-4.58 (m, 1H), 4.53-4.50 (m, 1H), 4.35 (s, 1H), 4.28-4.26 (m, 1H), 4.21-4.19 (m, 1H), 4.07-4.04 (m, 2H), 3.67-3.58 (m, 2H), 2.46 (s, 3H), 2.32-2.29 (m, 2H), 2.12-2.07 (m, 1H), 1.95-1.89 (m, 1H), 1.77-1.72 (m, 2H), 1.54-1.38 (m, 6H), 1.32-1.23 (m, 10H), 1.13 (s, 9H). MS (ESI) m/z=703.3 [M+H]⁺.

### Example 172: 11-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)undecanoic acid (Handle 61)

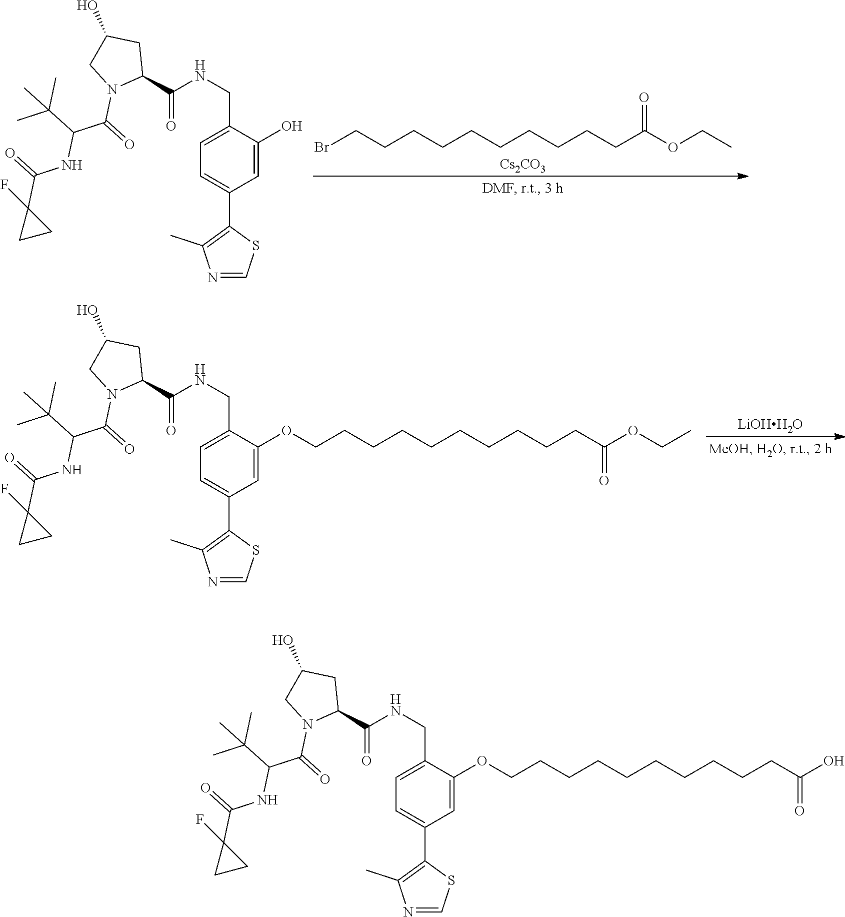

Handle 61 was synthesized following the same procedure for preparing Handle 52 (107 mg, yield: 64.3%). ¹H NMR (400 MHz, DMSO-d₆) δ 11.98 (s, 1H), 8.98 (s, 1H), 8.50-8.47 (m, 1H), 7.42 (d, J=8.0 Hz, 1H), 7.27 (d, J=11.6 Hz, 1H), 6.99 (s, 1H), 6.84 (d, J=7.6 Hz, 1H), 5.17 (s, 1H), 4.61-4.58 (m, 1H), 4.53-4.50 (m, 1H), 4.35 (s, 1H), 4.28-4.26 (m, 1H), 4.21-4.19 (m, 1H), 4.07-4.04 (m, 2H), 3.67-3.58 (m, 2H), 2.46 (s, 3H), 2.32-2.29 (m, 2H), 2.12-2.07 (m, 1H), 1.95-1.89 (m, 1H), 1.77-1.72 (m, 2H), 1.54-1.38 (m, 6H), 1.32-1.23 (m, 12H), 1.13 (s, 9H). MS (ESI) m/z=717.4 [M+H]⁻.

### Example 173: 6-((5-(4-(3-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-3-oxopropanoyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-111)

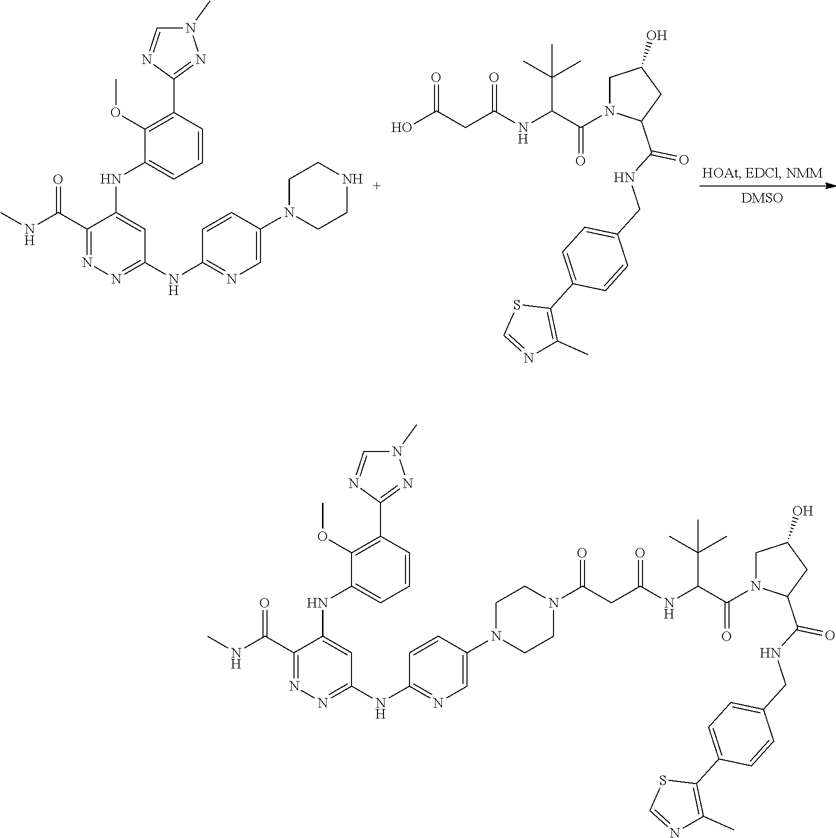 [SMILES: CNC(=O)c1nnc(Nc2ccc(N3CCNCC3)cn2)cc1Nc1cccc(-c2ncn(C)n2)c1OC ; Cc1ncsc1-c1ccc(CNC(=O)C2C[C@@H](O)CN2C(=O)C(NC(=O)CC(=O)O)C(C)(C)C)cc1 ; CNC(=O)c1nnc(Nc2ccc(N3CCN(C(=O)CC(=O)NC(C(=O)N4C[C@H](O)CC4C(=O)NCc4ccc(-c5scnc5C)cc4)C(C)(C)C)CC3)cn2)cc1Nc1cccc(-c2ncn(C)n2)c1OC]

CPD-111 was synthesized following the same procedure for preparing CPD-096 (2.2 mg, yield: 11.7%). MS (ESI) m/z=1015.0 [M+H]⁺.

### Example 174: 6-((5-(4-(8-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctanoyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-112)

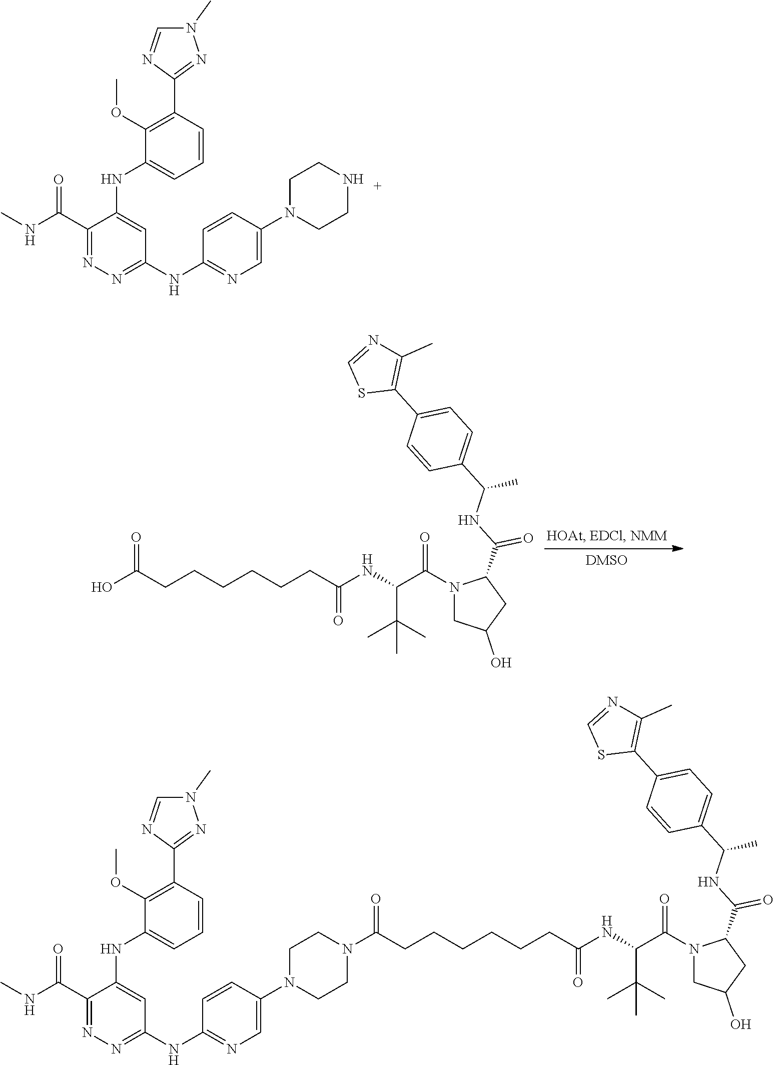 [SMILES: CNC(=O)c1nnc(Nc2ccc(N3CCNCC3)cn2)cc1Nc1cccc(-c2ncn(C)n2)c1OC ; Cc1ncsc1-c1ccc([C@H](C)NC(=O)[C@@H]2CC(O)CN2C(=O)[C@@H](NC(=O)CCCCCCC(=O)O)C(C)(C)C)cc1 ; CNC(=O)c1nnc(Nc2ccc(N3CCN(C(=O)CCCCCCC(=O)N[C@H](C(=O)N4CC(O)C[C@H]4C(=O)N[C@@H](C)c4ccc(-c5scnc5C)cc4)C(C)(C)C)CC3)cn2)cc1Nc1cccc(-c2ncn(C)n2)c1OC]

CPD-112 was synthesized following the same procedure for preparing CPD-096 (8.5 mg, yield: 26.5%). MS (ESI) m/z=1099.0 [M+H]⁺.

### Example 175: 6-((5-(4-(8-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-113)

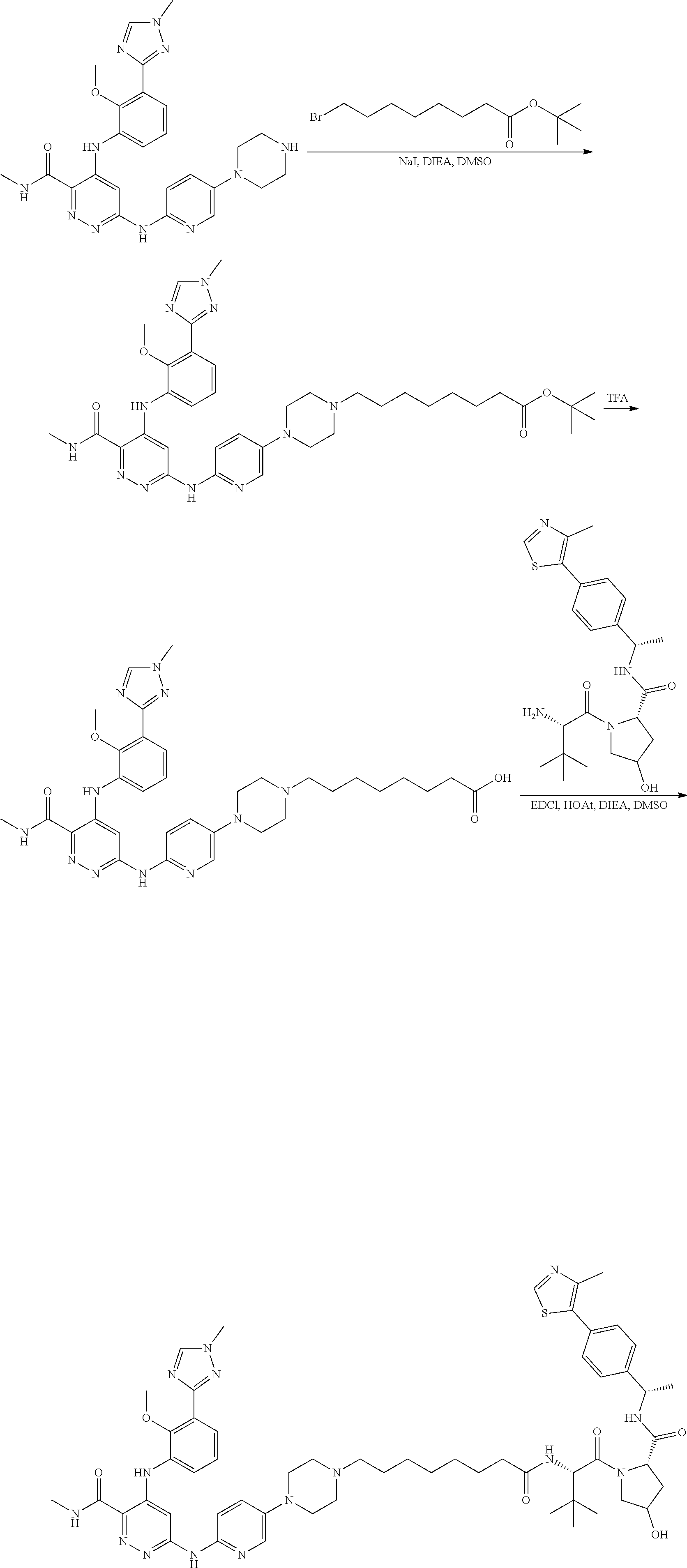

### Step 1. Synthesis of tert-butyl 8-(4-(6-((5-((2-methoxy-3-(1-methyl-1H-1,2,4-triazol-3-yl)phenyl)amino)-6-(methylcarbamoyl)pyridazin-3-yl)amino)pyridin-3-yl)piperazin-1-yl)octanoate

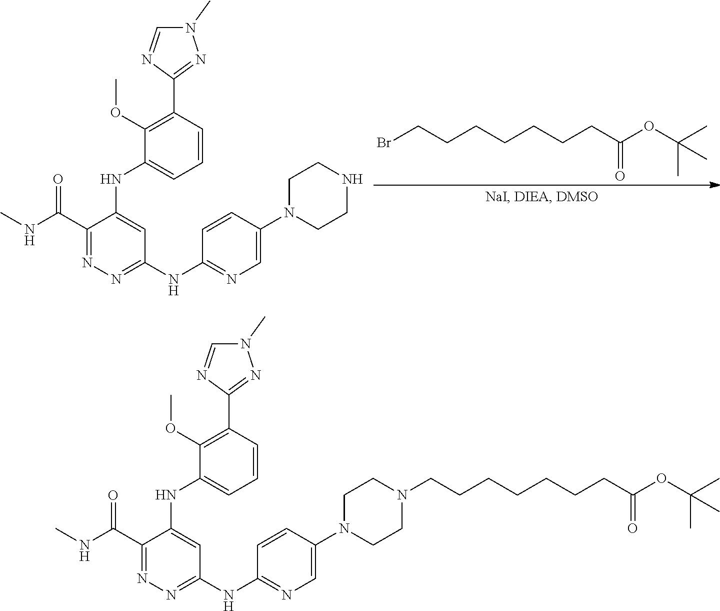 [SMILES: CNC(=O)c1nnc(Nc2ccc(N3CCNCC3)cn2)cc1Nc1cccc(-c2ncn(C)n2)c1OC ; CC(C)(C)OC(=O)CCCCCCCBr ; CNC(=O)c1nnc(Nc2ccc(N3CCN(CCCCCCCC(=O)OC(C)(C)C)CC3)cn2)cc1Nc1cccc(-c2ncn(C)n2)c1OC]

To a solution of 4-((2-methoxy-3-(1-methyl-1H-1,2,4-triazol-3-yl)phenyl)amino)-N-methyl-6-((5-(piperazin-1-yl)pyridin-2-yl)amino)pyridazine-3-carboxamide (30 mg, 0.058 mmol) in DMSO (2.5 mL) were added tert-butyl 8-bromooctanoate (23.2 mg, 0.087 mmol), DIEA (37.4 mg, 0.29 mmol) and NaI (43.5 mg, 0.29 mmol). After the reaction mixture was stirred at rt for 3.5 h, it was directly purified by prep-HPLC to afford the title compound (15 mg, 36.6% yield) as a yellow solid. MS (ESI) m/z=713.8 [M+H]⁺.

### Step 2. Synthesis of 8-(4-(6-((5-((2-methoxy-3-(1-methyl-1H-1,2,4-triazol-3-yl)phenyl)amino)-6-(methylcarbamoyl)pyridazin-3-yl)amino)pyridin-3-yl)piperazin-1-yl)octanoic acid

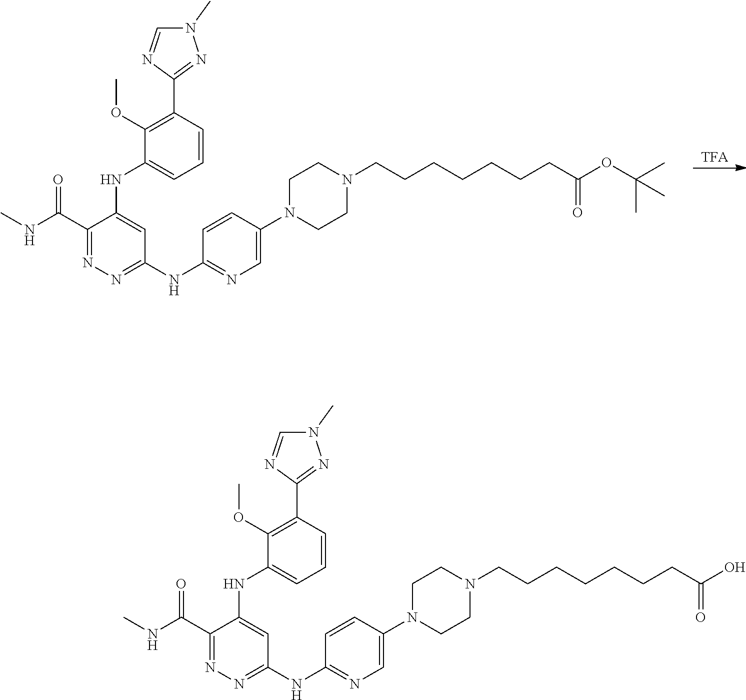

To a solution of tert-butyl 8-(4-(6-((5-((2-methoxy-3-(1-methyl-1H-1,2,4-triazol-3-yl)phenyl)amino)-6-(methylcarbamoyl)pyridazin-3-yl)amino)pyridin-3-yl)piperazin-1-yl)octanoate (15 mg, 0.021 mmol) in DCM (10 mL) was added TFA (5 mL). After the reaction mixture was stirred at rt for 2 h, it was concentrated and purified by prep-HPLC to give the title compound (12 mg, 87.6% yield) as a yellow solid. MS (ESI) m/z=657.8 [M+H]⁺.

### Step 3. Synthesis of 6-((5-(4-(8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide

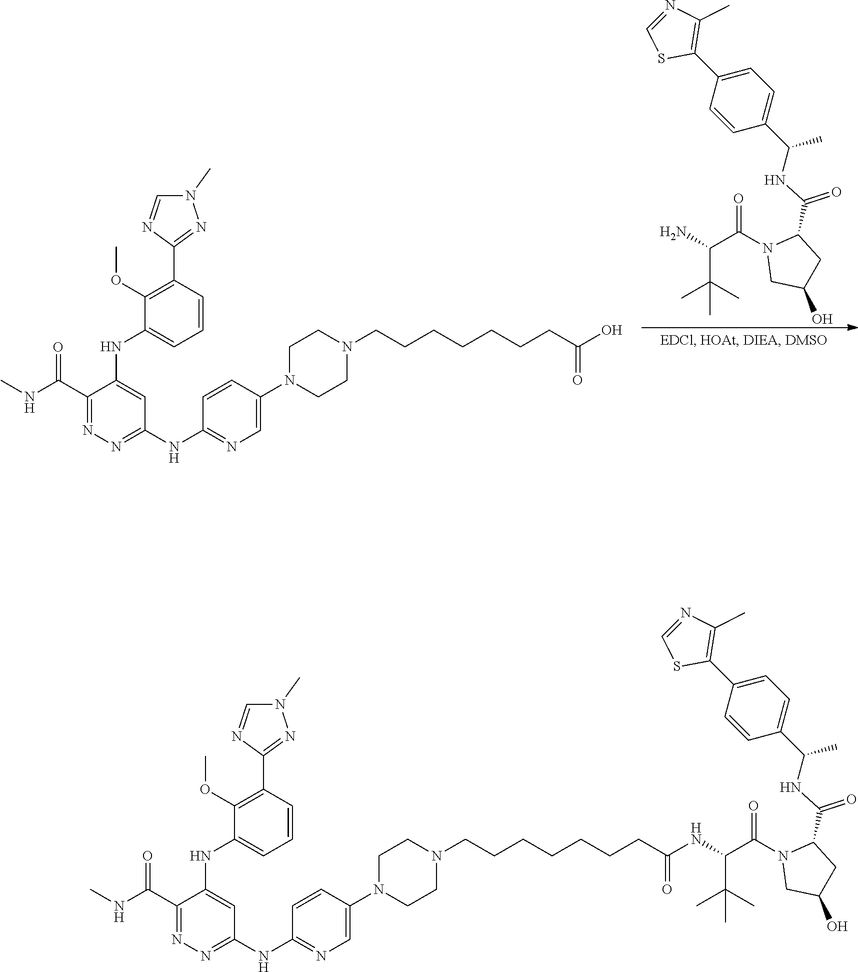

To a solution of 8-(4-(6-((5-((2-methoxy-3-(1-methyl-1H-1,2,4-triazol-3-yl)phenyl)amino)-6-(methylcarbamoyl)pyridazin-3-yl)amino)pyridin-3-yl)piperazin-1-yl)octanoic acid (12 mg, 18.2 μmol) in DMSO (2.5 mL) were added (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (9.69 mg, 21.8 μmol), HOAt (6.19 mg, 45.5 mol), EDCI (8.69 mg, 45.5 μmol) and DIEA (11.7 mg, 91 μmol). After the reaction mixture was stirred at rt overnight, it was diluted with H₂O (10 mL) and extracted with ethyl acetate (10 mL×3). The combined organic layers were washed with brine, dried over Na₂SO₄, filtered, and concentrated under reduced pressure. The residue was purified by flash chromatography to give the title compound (6.5 mg, 33% yield) as a yellow solid. MS (ESI) m/z=1085.0 [M+H]⁺.

### Example 176: 6-((5-(4-(10-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-114)

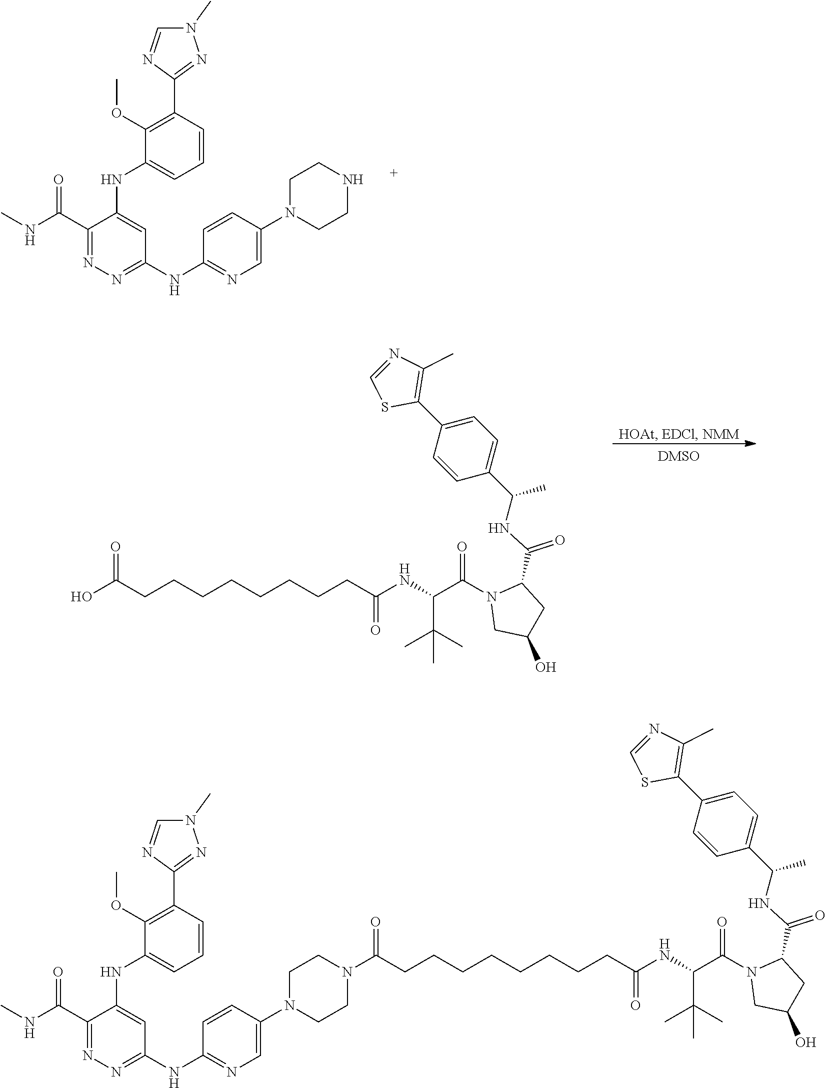

CPD-114 was synthesized following the same procedure for preparing CPD-096 (4.5 mg, yield: 8.3%). MS (ESI) m/z=1127.1 [M+H]⁺.

### Example 177: 6-((5-(4-(9-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononanoyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-115)

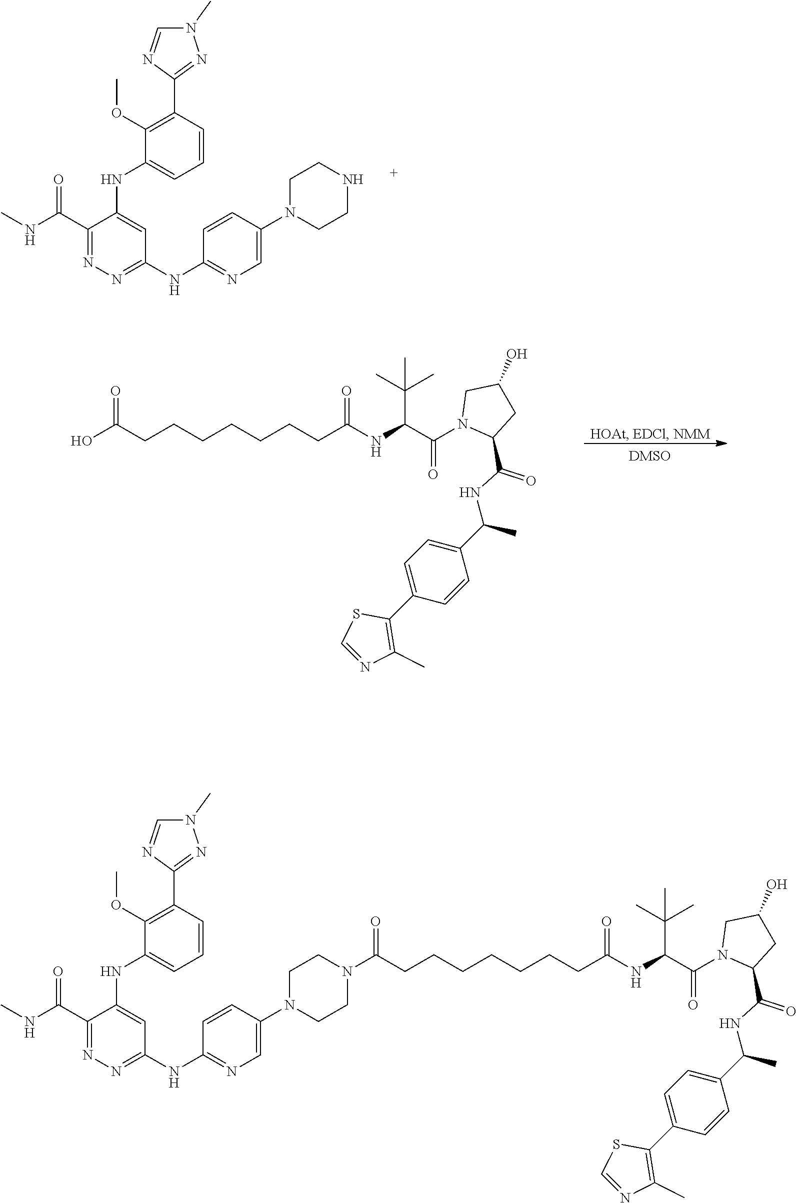

CPD-115 was synthesized following the same procedure for preparing CPD-096 (6.5 mg, yield: 36.7%). MS (ESI) m/z=1113.1 [M+H]⁺.

### Example 178: 6-((5-((1-(4-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutanoyl)piperidin-4-yl)ethynyl)pyridin-2-yl)amino)-4-((2-methoxy-3-(1-methyl-1H-1,2,4-triazol-3-yl)phenyl)amino)-N-methylpyridazine-3-carboxamide (CPD-116)

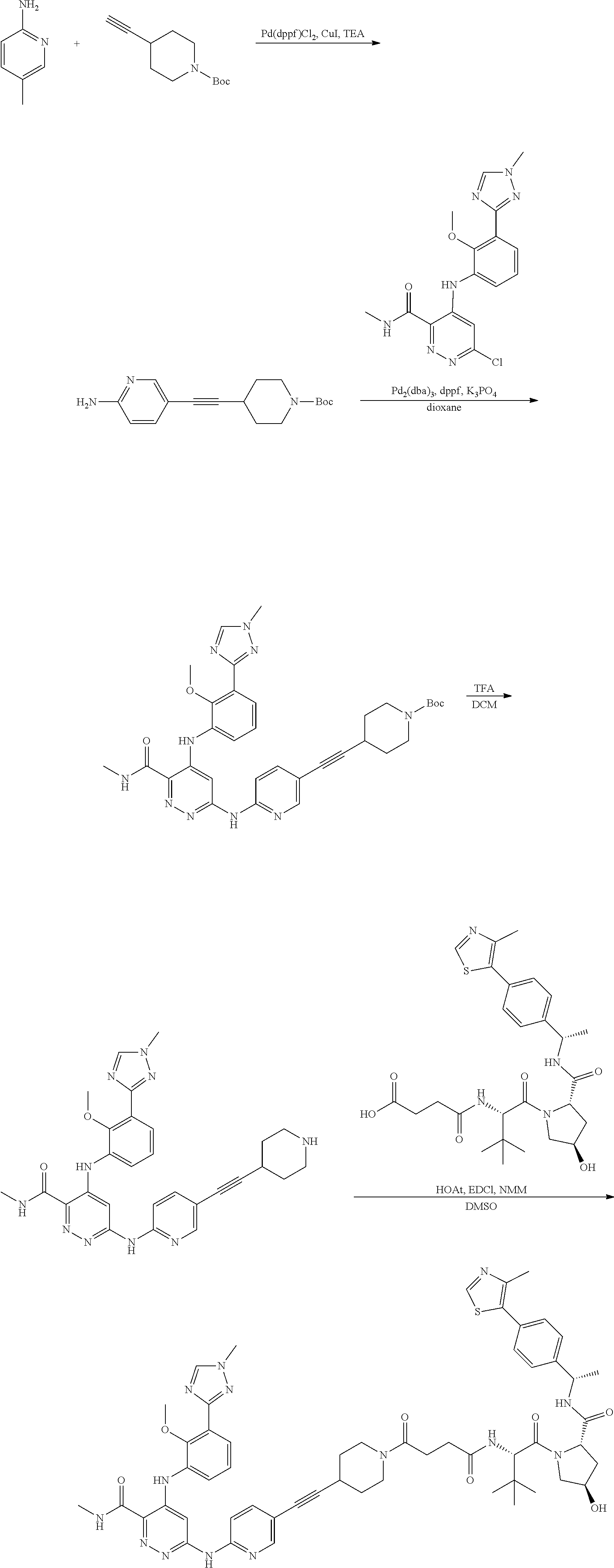

### Step 1. Synthesis of tert-butyl 4-((6-aminopyridin-3-yl)ethynyl)piperidine-1-carboxylate

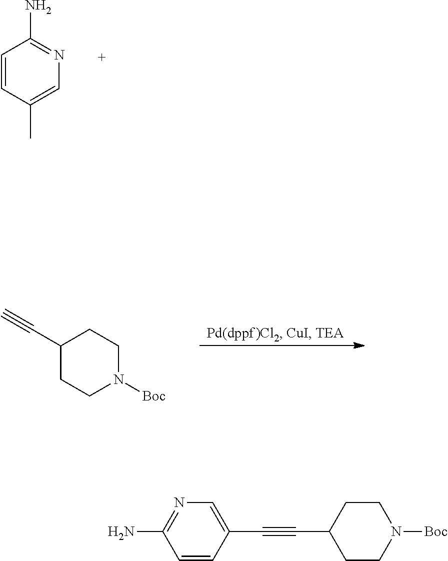

To a mixture of 5-iodopyridin-2-amine (440 mg, 2 mmol) and CuI (76 g, 0.4 mmol) in DMSO (10 mL) were added tert-butyl 4-ethynylpiperidine-1-carboxylate (502 mg, 2.4 mmol), Pd(dppf)Cl₂ (15 mg, 0.02 mmol) and TEA (606 mg, 6 mmol). After the mixture was stirred at 110° C. for 3 h, it was diluted with water and extracted with EtOAc (50 mL×3). The combined organic layers were washed with brine (30 mL×2), dried over Na₂SO₄, filtered, and concentrated under reduced pressure. The residue was purified by silica gel chromatography to give the title compound (454 mg, yield: 75.4%) as a light-brown solid. MS (ESI) m/z=302.3 [M+H]⁺.

### Step 2. Synthesis of tert-butyl 4-((6-((5-((2-methoxy-3-(1-methyl-1H-1,2,4-triazol-3-yl)phenyl)amino)-6-(methylcarbamoyl)pyridazin-3-yl)amino)pyridin-3-yl)ethynyl)piperidine-1-carboxylate

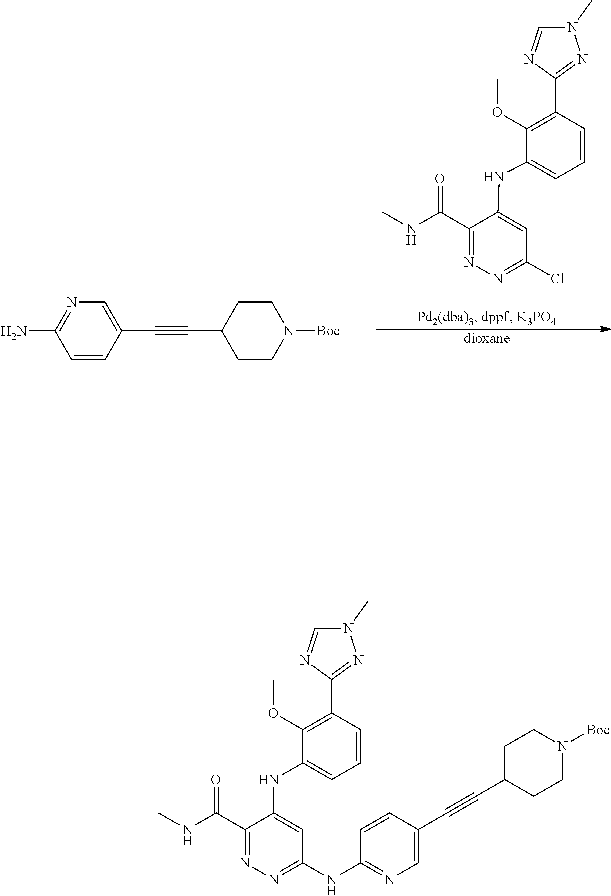

To a mixture of 6-chloro-4-((2-methoxy-3-(1-methyl-1H-1,2,4-triazol-3-yl)phenyl)amino)-N-methylpyridazine-3-carboxamide (310 mg, 0.83 mmol) and K₃PO₄ (528 mg, 2.49 mmol) in dioxane (10 mL) were added tert-butyl 4-((6-aminopyridin-3-yl)ethynyl)piperidine-1-carboxylate (300 mg, 1 mmol), Pd₂(dba)₃ (16 mg, 0.017 mmol) and dppf (19 mg, 0.034 mmol). After the resulting mixture was stirred at 100° C. for 16 h, it was diluted with water and extracted with EtOAc (40 mL×3). The combined organic layers were washed with brine (20 mL×2), dried over Na₂SO₄, filtered, and concentrated under reduced pressure. The residue was purified by reverse-phase chromatography to give the title compound (293 mg, yield: 55.3%) as an off-white solid. MS (ESI) m/z=639.5 [M+H]⁺.

### Step 3. Synthesis of 4-((2-methoxy-3-(1-methyl-1H-1,2,4-triazol-3-yl)phenyl)amino)-N-methyl-6-((5-(piperidin-4-ylethynyl)pyridin-2-yl)amino)pyridazine-3-carboxamide

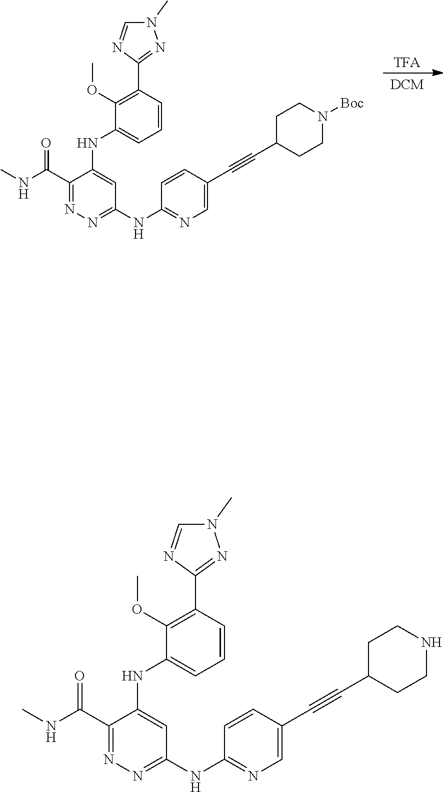

To a mixture of tert-butyl 4-((6-((5-((2-methoxy-3-(1-methyl-1H-1,2,4-triazol-3-yl)phenyl)amino)-6-(methylcarbamoyl)pyridazin-3-yl)amino)pyridin-3-yl)ethynyl)piperidine-1-carboxylate (293 mg, 0.46 mmol) in DCM (5 mL) was added TFA (5 mL). After the resulting mixture was stirred at rt for 16 h, it was concentrated under reduced pressure. The residue was purified by reverse-phase chromatography to give the title compound (185 mg, yield: 74.9%) as an off-white solid. MS (ESI) m/z=539.4 [M+H]⁺.

### Step 4. Synthesis of 6-((5-((1-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutanoyl)piperidin-4-yl)ethynyl)pyridin-2-yl)amino)-4-((2-methoxy-3-(1-methyl-1H-1,2,4-triazol-3-yl)phenyl)amino)-N-methylpyridazine-3-carboxamide

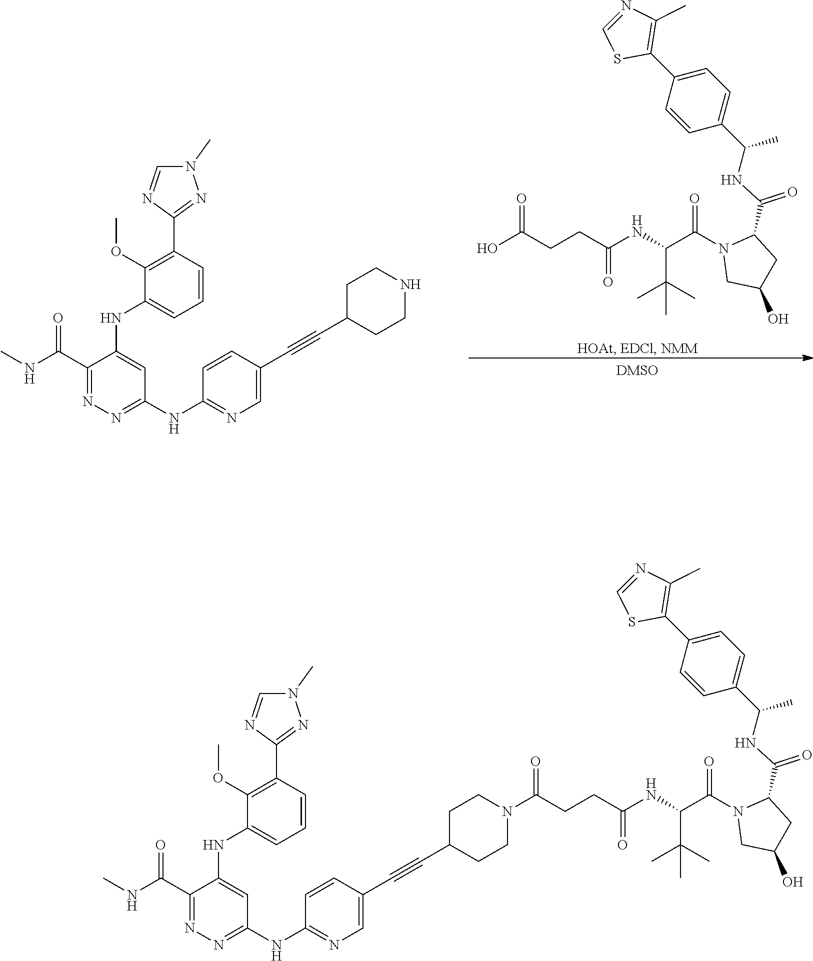

CPD-116 was synthesized following the same procedure for preparing CPD-042 (4.3 mg, yield: 45.2%). MS (ESI) m/z=1066.0 [M+H]⁺.

### Example 179: 6-((5-((1-(6-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexanoyl)piperidin-4-yl)ethynyl)pyridin-2-yl)amino)-4-((2-methoxy-3-(1-methyl-1H-1,2,4-triazol-3-yl)phenyl)amino)-N-methylpyridazine-3-carboxamide (CPD-117)

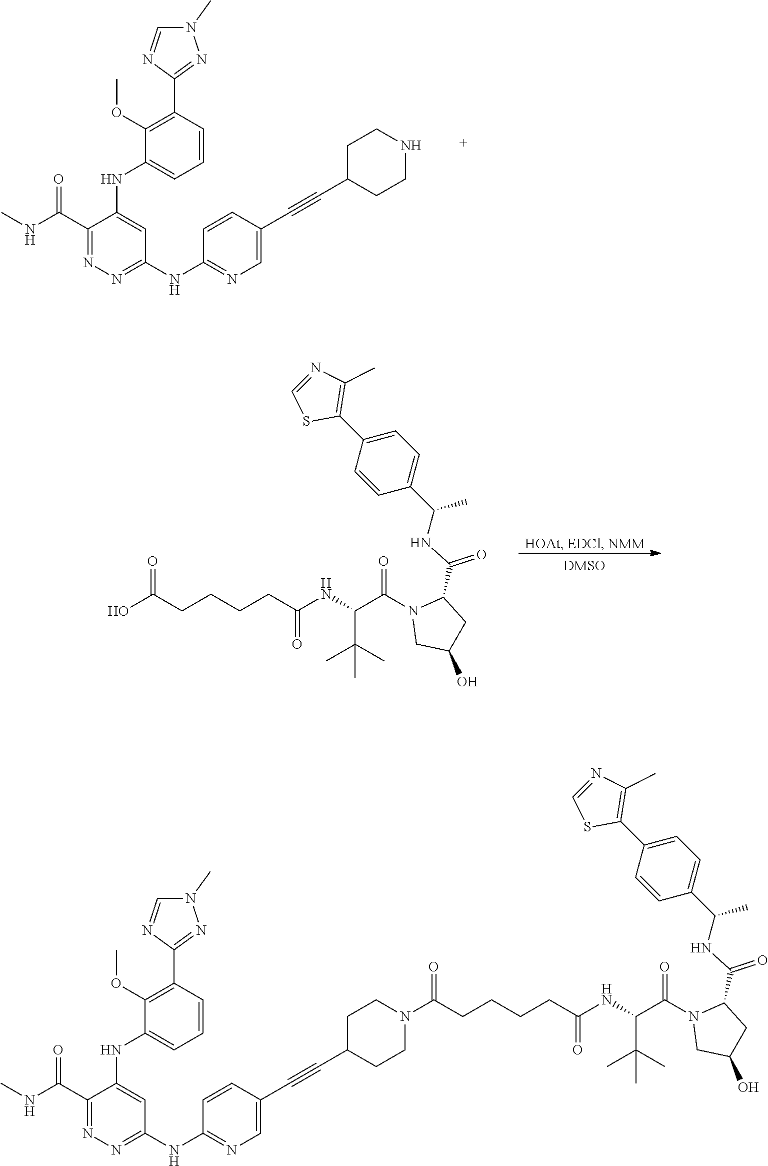

CPD-117 was synthesized following the same procedure for preparing CPD-116 (8.8 mg, yield: 53.5%). MS (ESI) m/z=1094.1 [M+H]⁺.

### Example 180: 6-((5-((1-(7-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoyl)piperidin-4-yl)ethynyl)pyridin-2-yl)amino)-4-((2-methoxy-3-(1-methyl-1H-1,2,4-triazol-3-yl)phenyl)amino)-N-methylpyridazine-3-carboxamide (CPD-118)

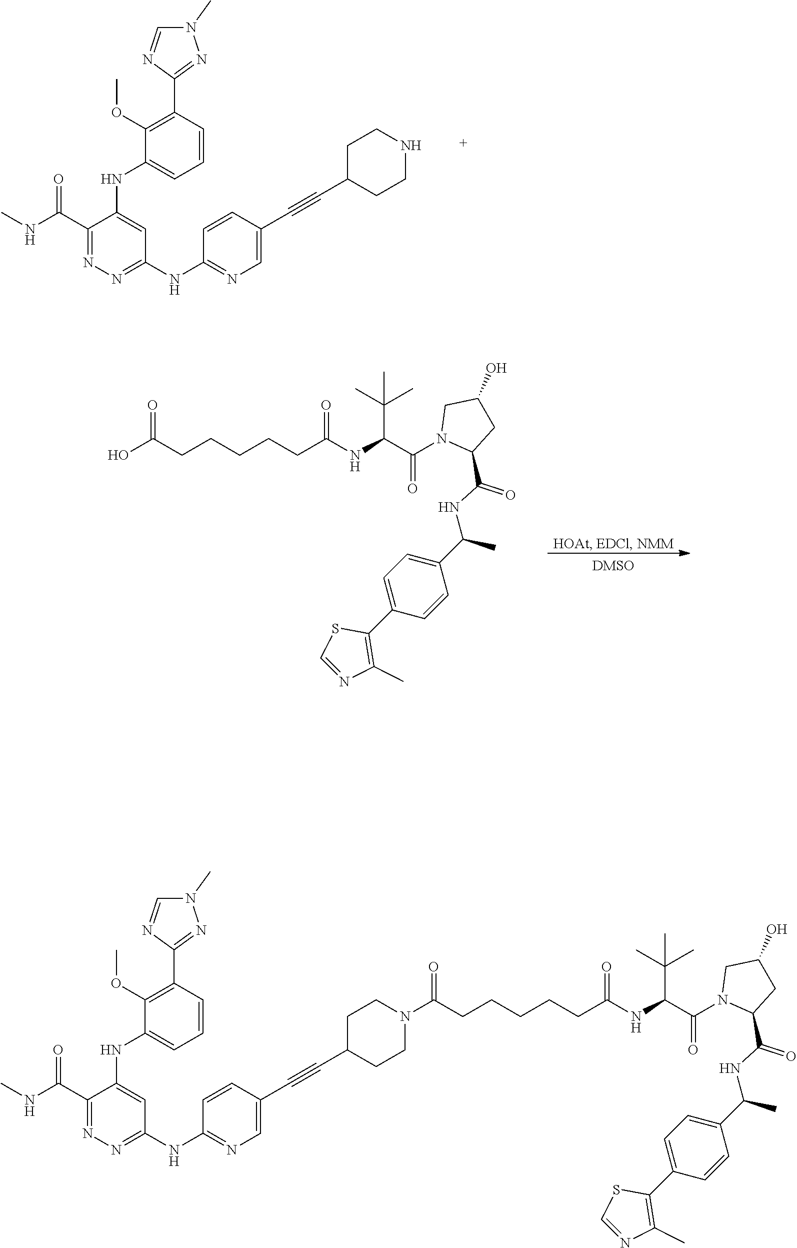

CPD-118 was synthesized following the same procedure for preparing CPD-116 (4.3 mg, yield: 23%). MS (ESI) m/z=1108.1 [M+H]⁺.

### Example 181: 6-((5-((1-(8-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctanoyl)piperidin-4-yl)ethynyl)pyridin-2-yl)amino)-4-((2-methoxy-3-(1-methyl-1H-1,2,4-triazol-3-yl)phenyl)amino)-N-methylpyridazine-3-carboxamide (CPD-119)

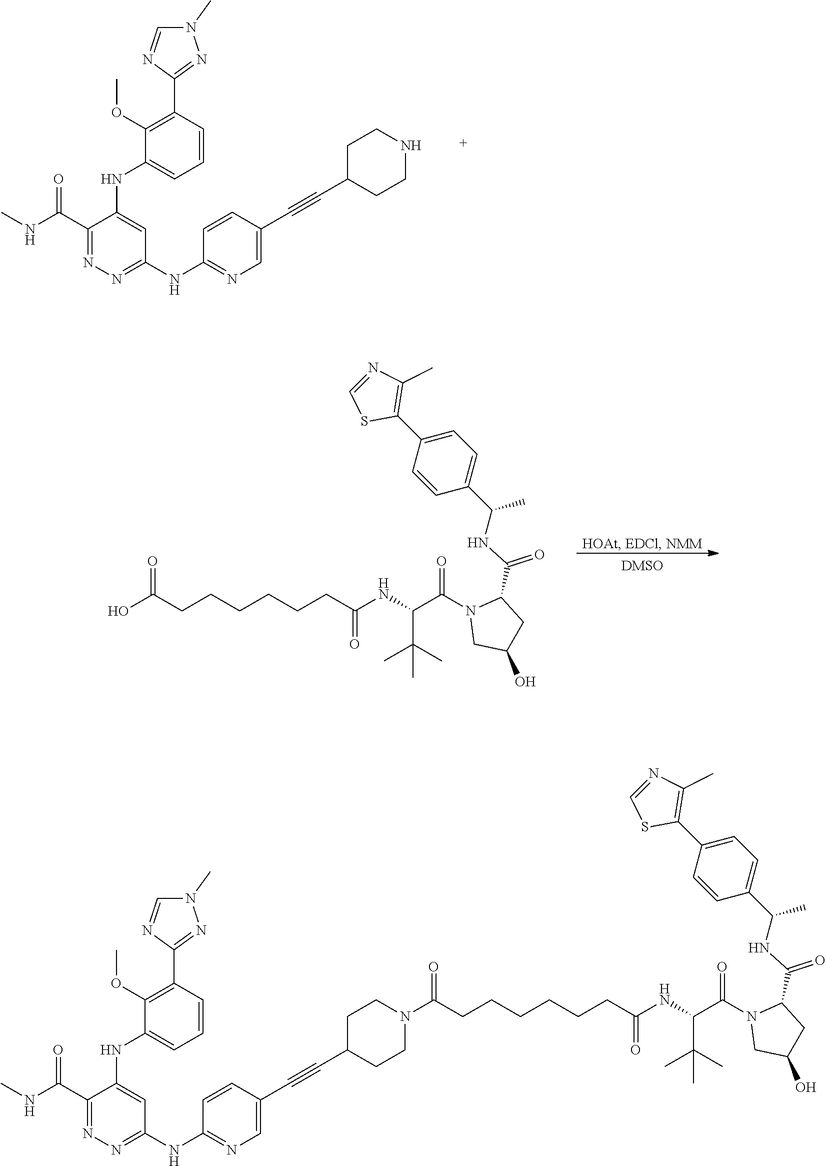

CPD-119 was synthesized following the same procedure for preparing CPD-116 (5.9 mg, yield: 35%). MS (ESI) m/z=1122.2 [M+H]⁺.

### Example 182: 6-((5-((1-(9-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononanoyl)piperidin-4-yl)ethynyl)pyridin-2-yl)amino)-4-((2-methoxy-3-(1-methyl-1H-1,2,4-triazol-3-yl)phenyl)amino)-N-methylpyridazine-3-carboxamide (CPD-120)

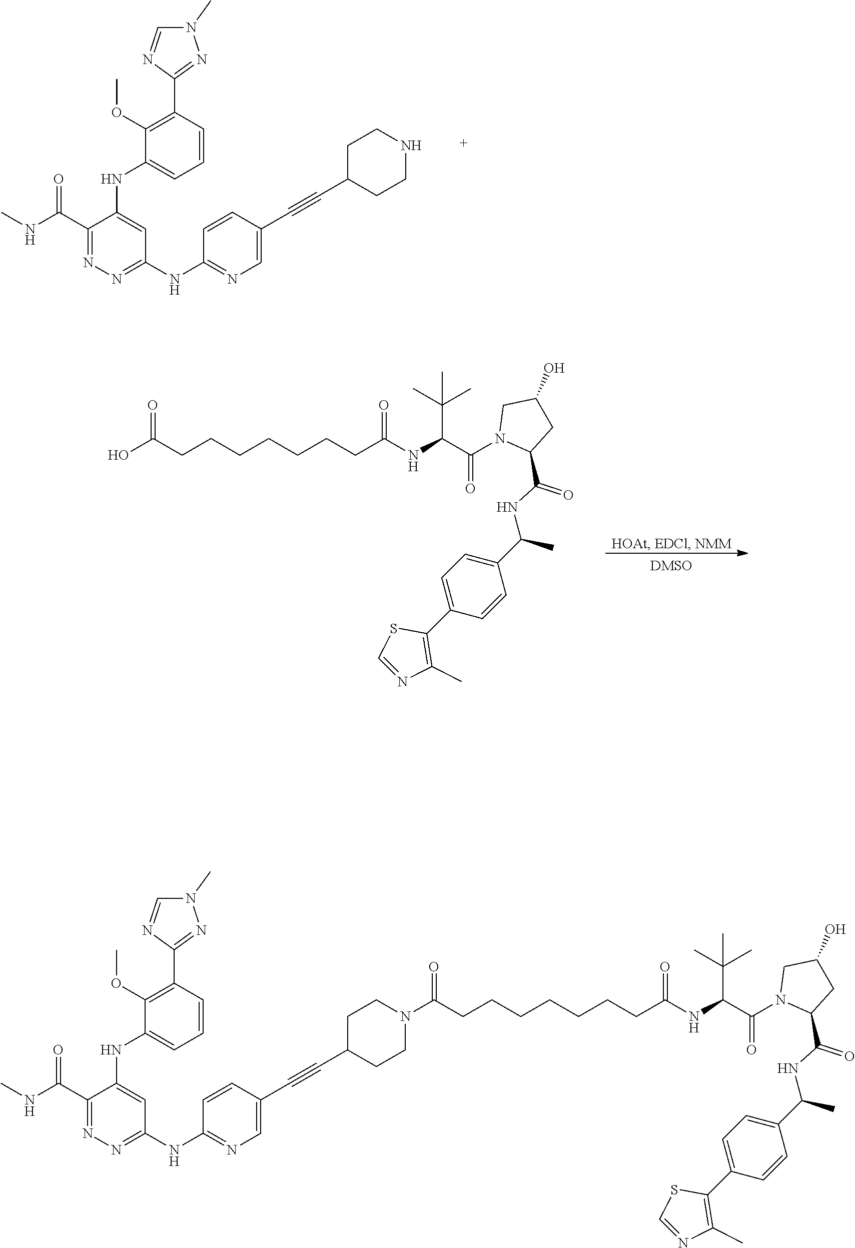

CPD-120 was synthesized following the same procedure for preparing CPD-116 (6.5 mg, yield: 36.2%). MS (ESI) m/z=1136.3 [M+H]⁺.

### Example 183: 6-((5-((1-(10-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoyl)piperidin-4-yl)ethynyl)pyridin-2-yl)amino)-4-((2-methoxy-3-(1-methyl-1H-1,2,4-triazol-3-yl)phenyl)amino)-N-methylpyridazine-3-carboxamide (CPD-121)

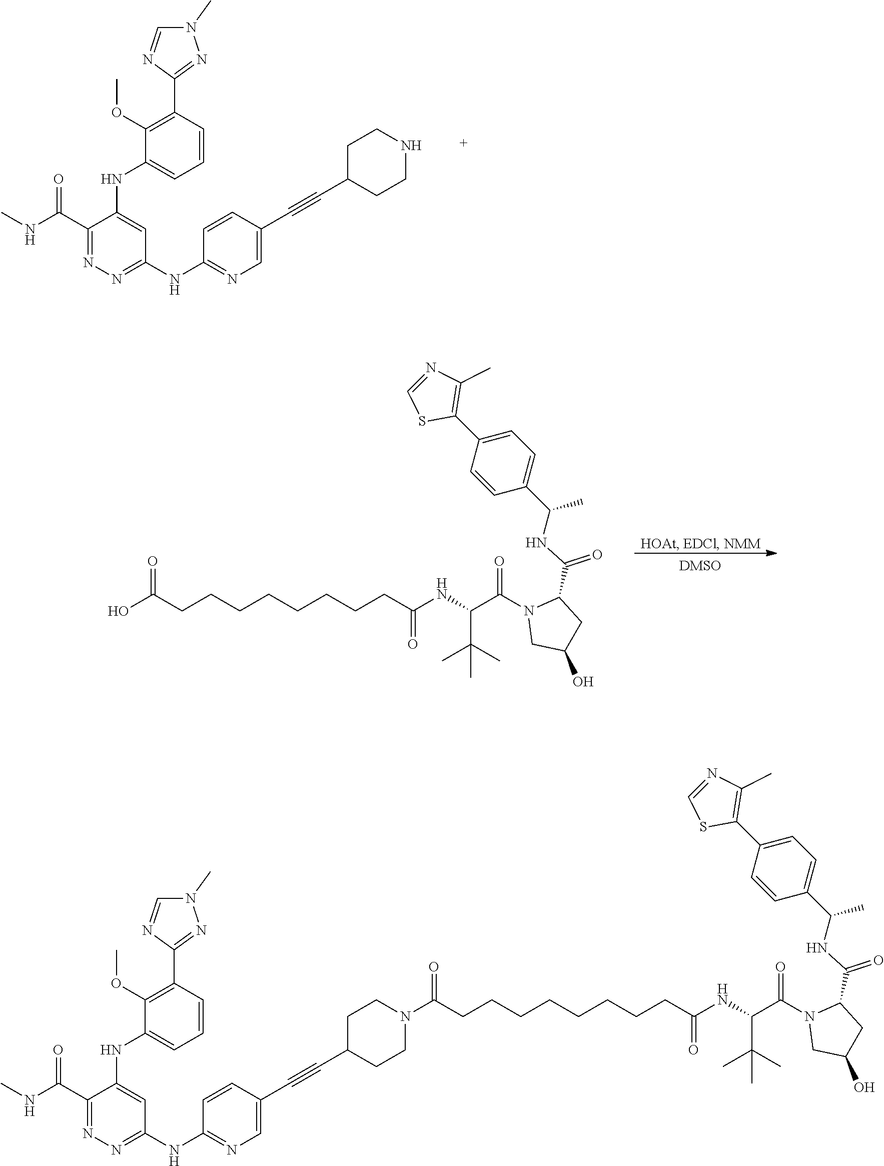

CPD-121 was synthesized following the same procedure for preparing CPD-116 (4.9 mg, yield: 30.4%). MS (ESI) m/z=1150.1 [M+H]⁺.

### Example 184: 6-((5-((1-(4-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutanoyl)azetidin-3-yl)ethynyl)pyridin-2-yl)amino)-4-((2-methoxy-3-(1-methyl-1H-1,2,4-triazol-3-yl)phenyl)amino)-N-methylpyridazine-3-carboxamide (CPD-122)

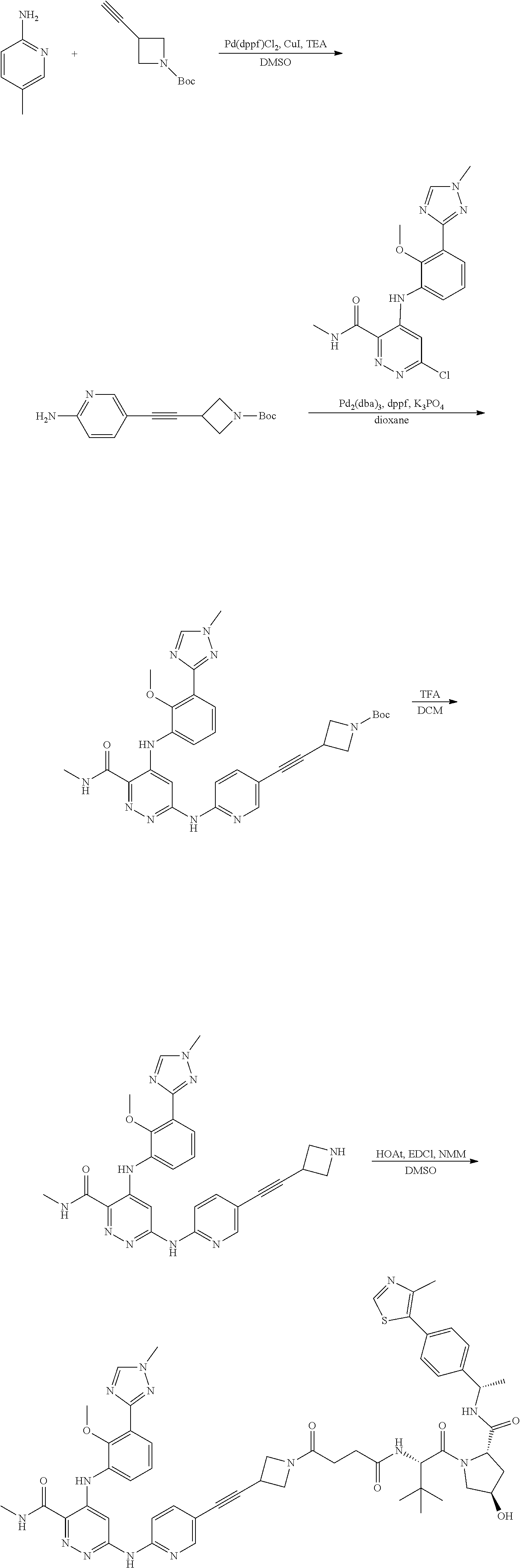

CPD-122 was synthesized following the same procedure for preparing CPD-116 (3.7 mg, yield: 36.6%). MS (ESI) m/z=1038.0 [M+H]⁺.

### Example 185: 6-((5-((1-(6-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexanoyl)azetidin-3-yl)ethynyl)pyridin-2-yl)amino)-4-((2-methoxy-3-(1-methyl-1H-1,2,4-triazol-3-yl)phenyl)amino)-N-methylpyridazine-3-carboxamide (CPD-123)

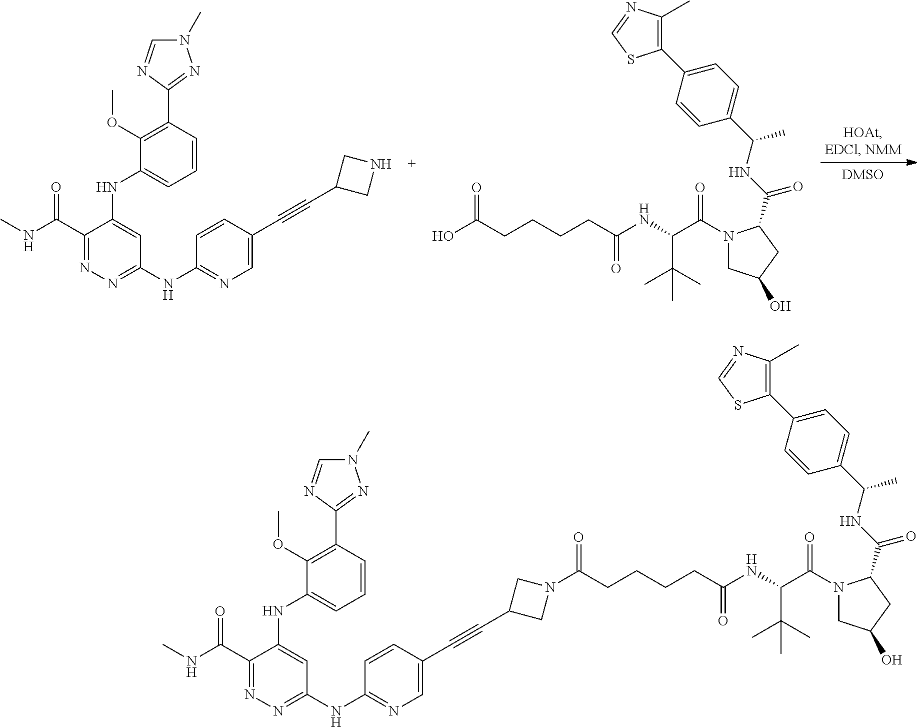

CPD-123 was synthesized following the same procedure for preparing CPD-116 (7.9 mg, yield: 49.5%). MS (ESI) m/z=1066.1 [M+H]⁺.

### Example 186: 6-((5-((1-(7-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoyl)azetidin-3-yl)ethynyl)pyridin-2-yl)amino)-4-((2-methoxy-3-(1-methyl-1H-1,2,4-triazol-3-yl)phenyl)amino)-N-methylpyridazine-3-carboxamide (CPD-124)

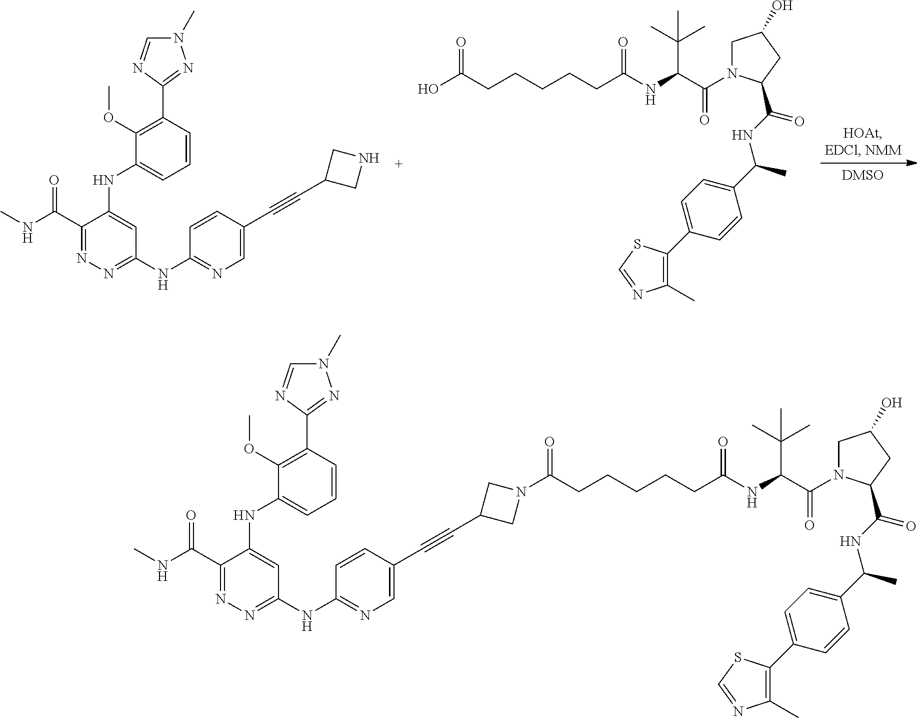 [SMILES: CNC(=O)c1nnc(Nc2ccc(C#CC3CNC3)cn2)cc1Nc1cccc(-c2ncn(C)n2)c1OC ; Cc1ncsc1-c1ccc([C@H](C)NC(=O)[C@@H]2C[C@@H](O)CN2C(=O)[C@@H](NC(=O)CCCCCC(=O)O)C(C)(C)C)cc1 ; CNC(=O)c1nnc(Nc2ccc(C#CC3CN(C(=O)CCCCCC(=O)N[C@H](C(=O)N4C[C@H](O)C[C@H]4C(=O)N[C@@H](C)c4ccc(-c5scnc5C)cc4)C(C)(C)C)C3)cn2)cc1Nc1cccc(-c2ncn(C)n2)c1OC]

CPD-124 was synthesized following the same procedure for preparing CPD-116 (5.4 mg, yield: 28%). MS (ESI) m/z=1080.1 [M+H]⁺.

### Example 187: 6-((5-((1-(8-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctanoyl)azetidin-3-yl)ethynyl)pyridin-2-yl)amino)-4-((2-methoxy-3-(1-methyl-H-1,2,4-triazol-3-yl)phenyl)amino)-N-methylpyridazine-3-carboxamide (CPD-125)

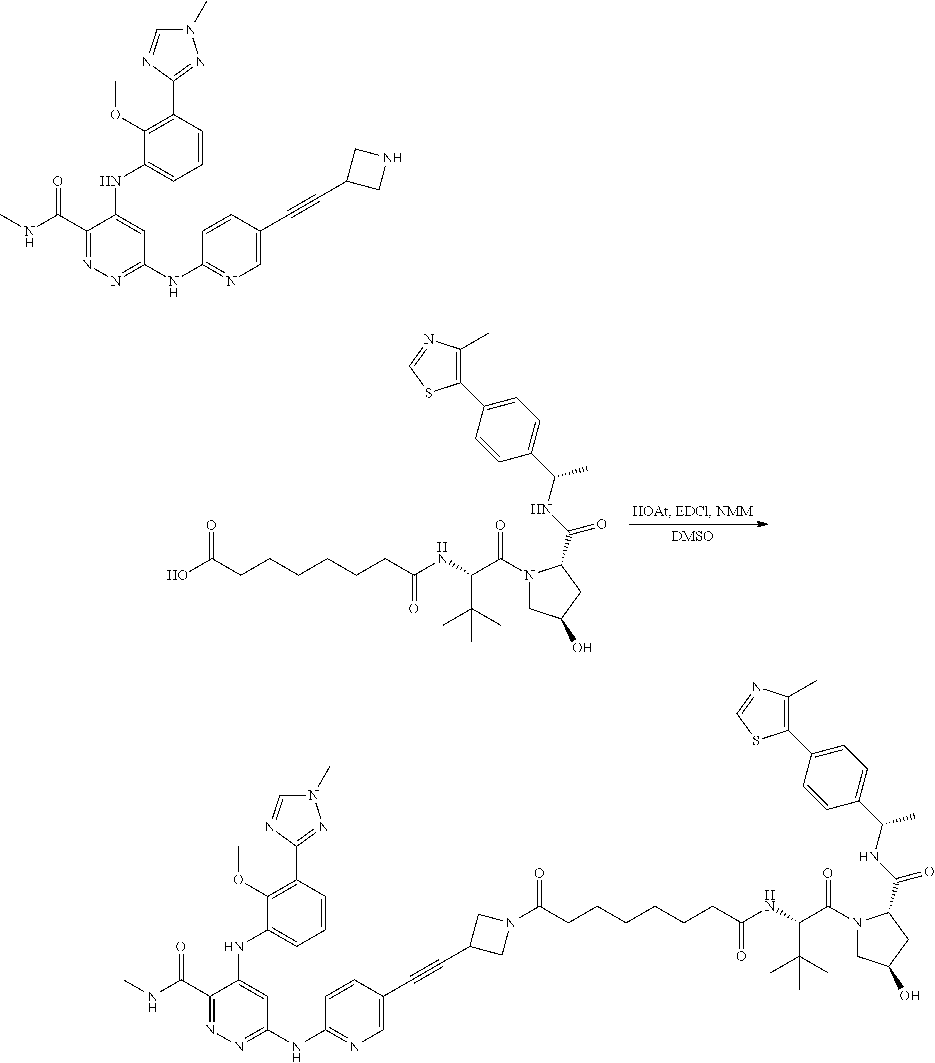 [SMILES: CNC(=O)c1nnc(Nc2ccc(C#CC3CNC3)cn2)cc1Nc1cccc(-c2ncn(C)n2)c1OC ; Cc1ncsc1-c1ccc([C@H](C)NC(=O)[C@@H]2C[C@@H](O)CN2C(=O)[C@@H](NC(=O)CCCCCCC(=O)O)C(C)(C)C)cc1 ; CNC(=O)c1nnc(Nc2ccc(C#CC3CN(C(=O)CCCCCCC(=O)N[C@H](C(=O)N4C[C@H](O)C[C@H]4C(=O)N[C@@H](C)c4ccc(-c5scnc5C)cc4)C(C)(C)C)C3)cn2)cc1Nc1cccc(-c2ncn(C)n2)c1OC]

CPD-125 was synthesized following the same procedure for preparing CPD-116 (2.2 mg, yield: 13.3%). MS (ESI) m/z=1094.1 [M+H]⁺.

### Example 188: 6-((5-((1-(9-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononanoyl)azetidin-3-yl)ethynyl)pyridin-2-yl)amino)-4-((2-methoxy-3-(1-methyl-1H-1,2,4-triazol-3-yl)phenyl)amino)-N-methylpyridazine-3-carboxamide (CPD-126)

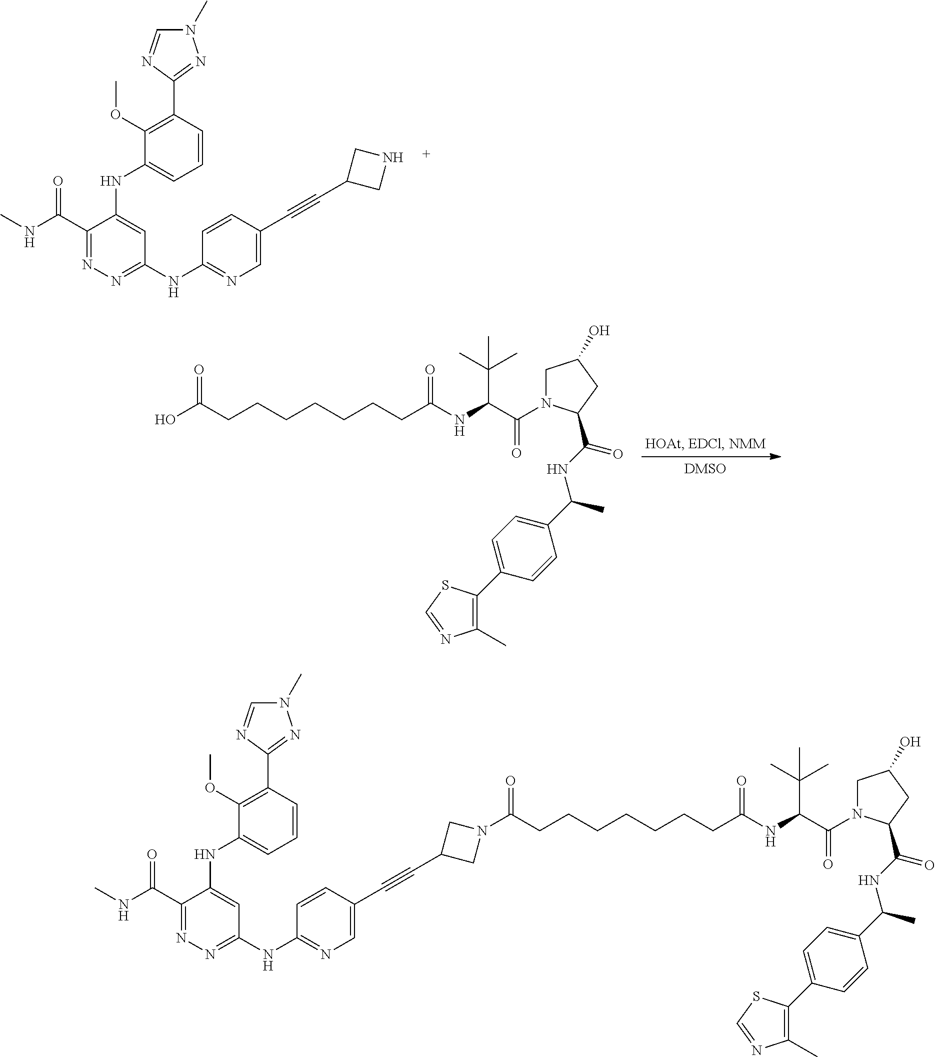 [SMILES: CNC(=O)c1nnc(Nc2ccc(C#CC3CNC3)cn2)cc1Nc1cccc(-c2ncn(C)n2)c1OC ; Cc1ncsc1-c1ccc([C@H](C)NC(=O)[C@@H]2C[C@@H](O)CN2C(=O)[C@@H](NC(=O)CCCCCCCC(=O)O)C(C)(C)C)cc1 ; CNC(=O)c1nnc(Nc2ccc(C#CC3CN(C(=O)CCCCCCCC(=O)N[C@H](C(=O)N4C[C@H](O)C[C@H]4C(=O)N[C@@H](C)c4ccc(-c5scnc5C)cc4)C(C)(C)C)C3)cn2)cc1Nc1cccc(-c2ncn(C)n2)c1OC]

CPD-126 was synthesized following the same procedure for preparing CPD-116 (4.3 mg, yield: 25.5%). MS (ESI) m/z=1107.7 [M+H]⁺.

### Example 189: 6-((5-((1-(10-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoyl)azetidin-3-yl)ethynyl)pyridin-2-yl)amino)-4-((2-methoxy-3-(1-methyl-1H-1,2,4-triazol-3-yl)phenyl)amino)-N-methylpyridazine-3-carboxamide (CPD-127)

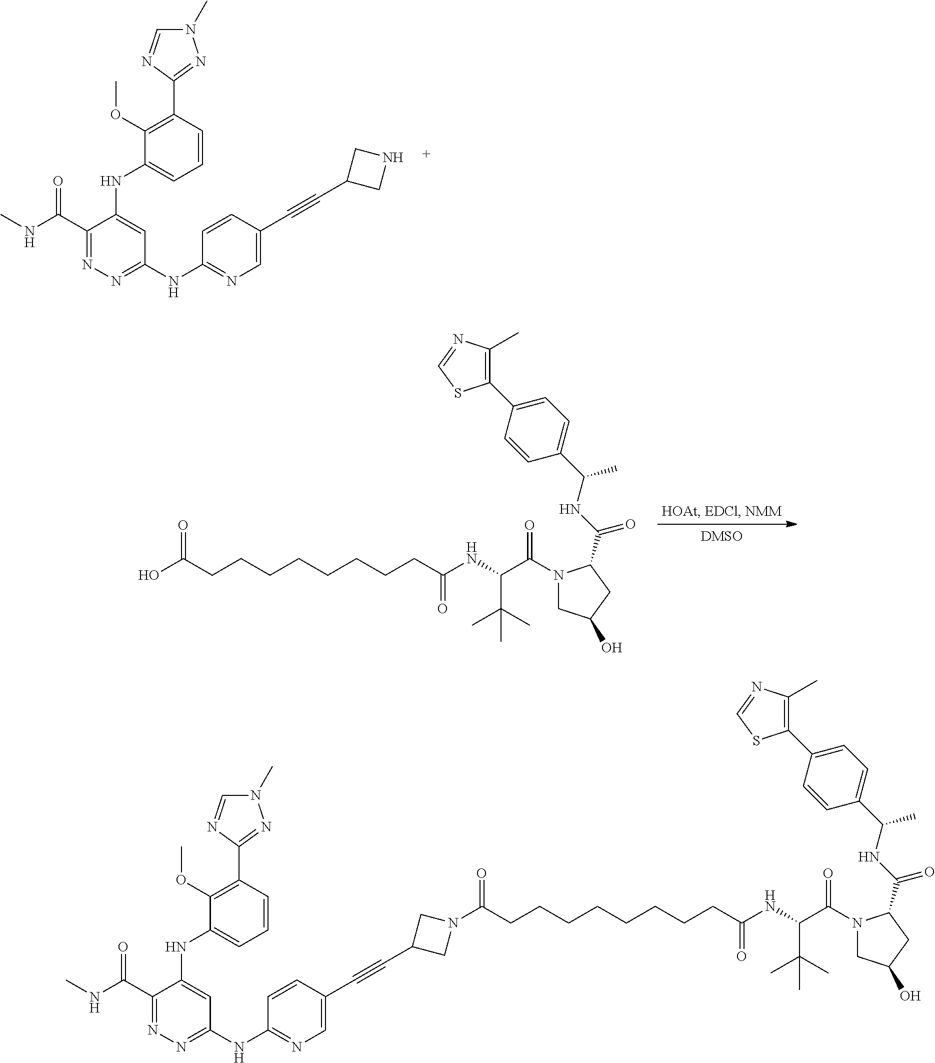 [SMILES: CNC(=O)c1nnc(Nc2ccc(C#CC3CNC3)cn2)cc1Nc1cccc(-c2ncn(C)n2)c1OC ; Cc1ncsc1-c1ccc([C@H](C)NC(=O)[C@@H]2C[C@@H](O)CN2C(=O)[C@@H](NC(=O)CCCCCCCCC(=O)O)C(C)(C)C)cc1 ; CNC(=O)c1nnc(Nc2ccc(C#CC3CN(C(=O)CCCCCCCCC(=O)N[C@H](C(=O)N4C[C@H](O)C[C@H]4C(=O)N[C@@H](C)c4ccc(-c5scnc5C)cc4)C(C)(C)C)C3)cn2)cc1Nc1cccc(-c2ncn(C)n2)c1OC]

CPD-127 was synthesized following the same procedure for preparing CPD-116 (5.5 mg, yield: 30.4%). MS (ESI) m/z=1122.0 [M+H]⁺.

### Example 190: N1-((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)-N4-((6-((5-((2-methoxy-3-(1-methyl-1H-1,2,4-triazol-3-yl)phenyl)amino)-6-(methylcarbamoyl)pyridazin-3-yl)amino)pyridin-3-yl)methyl)succinamide (CPD-128)

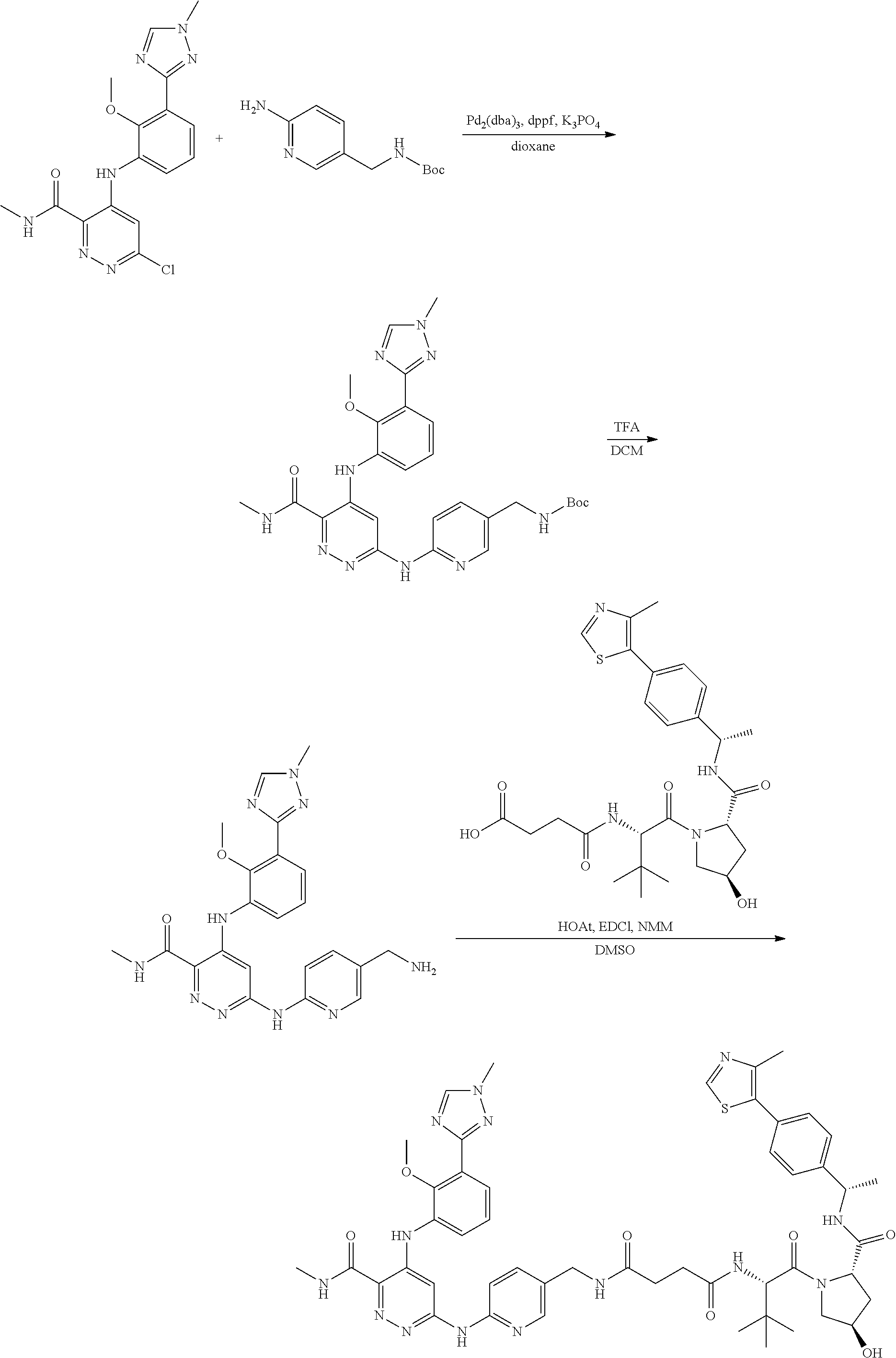

### Step 1. Synthesis of tert-butyl ((6-((5-((2-methoxy-3-(1-methyl-1H-1,2,4-triazol-3-yl)phenyl)amino)-6-(methylcarbamoyl)pyridazin-3-yl)amino)pyridin-3-yl)methyl)carbamate

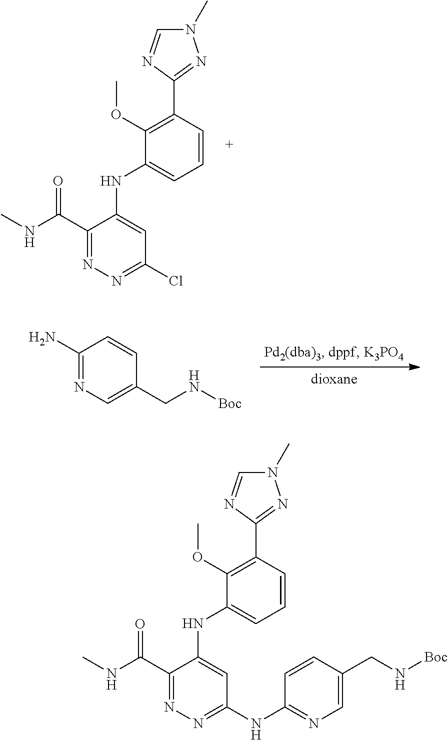

A mixture of 6-chloro-4-((2-methoxy-3-(1-methyl-1H-1,2,4-triazol-3-yl)phenyl)amino)-N-methylpyridazine-3-carboxamide (444 mg, 1.19 mmol) and K₃PO₄ (757 mg, 3.57 mmol) in dioxane (15 mL) were added tert-butyl ((6-aminopyridin-3-yl)methyl)carbamate (318 mg, 1.43 mmol), Pd₂(dba)₃ (23.3 mg, 0.024 mmol) and dppf (22 mg, 0.048 mmol). After the resulting mixture was stirred at 100° C. for 16 h, it was diluted with water and extracted with EtOAc (30 mL×3). The combined organic layers were combined and washed with brine (20 mL×2), dried over Na₂SO₄, filtered, and concentrated under reduced pressure. The residue was purified by reverse-phase chromatography to give the title compound (249 mg, yield: 37.4%) as a light yellow solid. MS (ESI) m/z=561.4 [M+H]⁺.

### Step 2. Synthesis of 6-((5-(aminomethyl)pyridin-2-yl)amino)-4-((2-methoxy-3-(1-methyl-1H-1,2,4-triazol-3-yl)phenyl)amino)-N-methylpyridazine-3-carboxamide

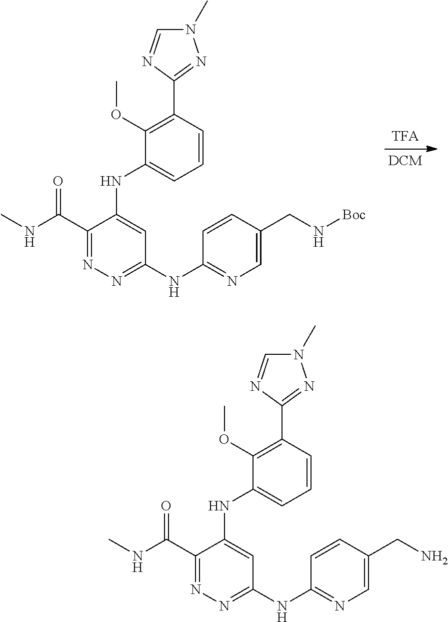 [SMILES: CNC(=O)c1nnc(Nc2ccc(CNC(=O)OC(C)(C)C)cn2)cc1Nc1cccc(-c2ncn(C)n2)c1OC ; CNC(=O)c1nnc(Nc2ccc(CN)cn2)cc1Nc1cccc(-c2ncn(C)n2)c1OC]

To a mixture of tert-butyl ((6-((5-((2-methoxy-3-(1-methyl-1H-1,2,4-triazol-3-yl)phenyl)amino)-6-(methylcarbamoyl)pyridazin-3-yl)amino)pyridin-3-yl)methyl)carbamate (249 mg, 0.44 mmol) in DCM (5 mL) was added TFA (5 mL). After the mixture was stirred at 25° C. for 16 h, it was concentrated under reduced pressure. The residue was purified by reverse-phase chromatography to give the title compound (188 mg, yield: 92.2%) as a light yellow solid.

### Step 3. Synthesis of N¹-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-N⁴-((6-((5-((2-methoxy-3-(1-methyl-1H-1,2,4-triazol-3-yl)phenyl)amino)-6-(methylcarbamoyl)pyridazin-3-yl)amino)pyridin-3-yl)methyl)succinamide

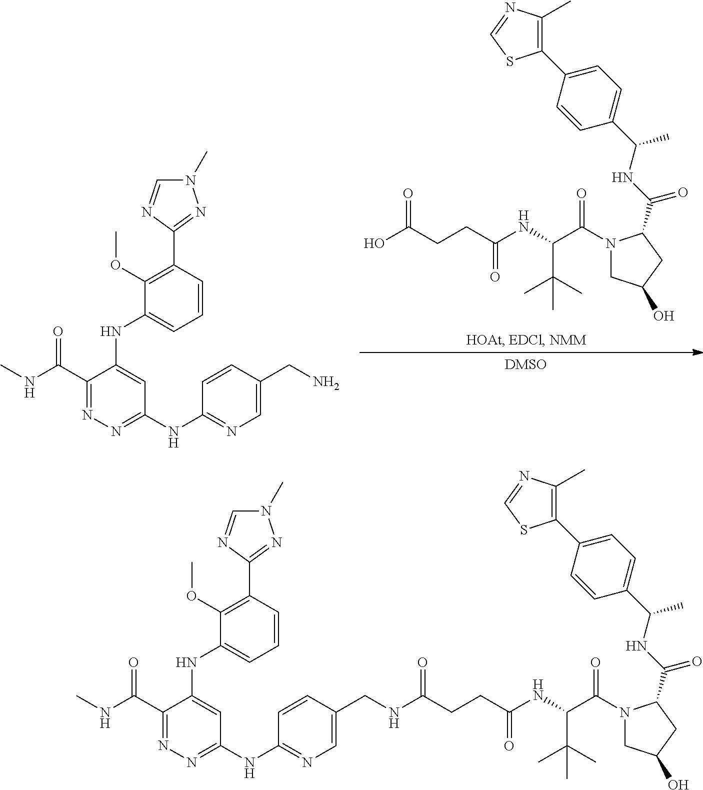 [SMILES: CNC(=O)c1nnc(Nc2ccc(CN)cn2)cc1Nc1cccc(-c2ncn(C)n2)c1OC ; Cc1ncsc1-c1ccc([C@H](C)NC(=O)[C@@H]2C[C@@H](O)CN2C(=O)[C@@H](NC(=O)CCC(=O)O)C(C)(C)C)cc1 ; CNC(=O)c1nnc(Nc2ccc(CNC(=O)CCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC]

CPD-128 was synthesized following the same procedure for preparing CPD-042 (5.1 mg, yield: 51.7%). MS (ESI) m/z=987.8 [M+H]⁺.

### Example 191: N1-((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)-N6-((6-((5-((2-methoxy-3-(1-methyl-1H-1,2,4-triazol-3-yl)phenyl)amino)-6-(methylcarbamoyl)pyridazin-3-yl)amino)pyridin-3-yl)methyl)adipamide (CPD-129)

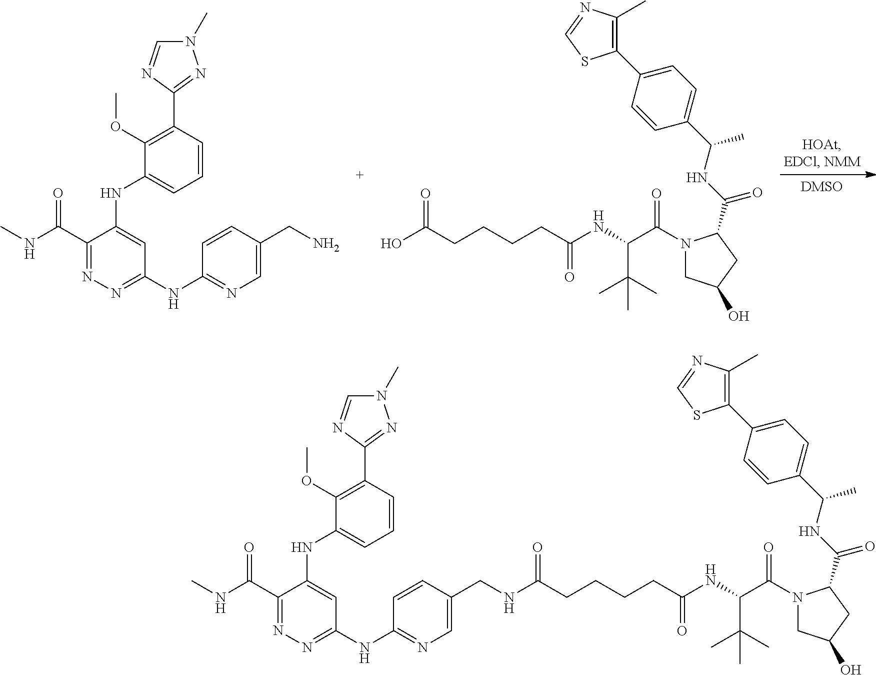

CPD-129 was synthesized following the same procedure for preparing CPD-128 (6.1 mg, yield: 39.7%). MS (ESI) m/z=1016.0 [M+H]⁺.

### Example 192: N1-((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)-N7-((6-((5-((2-methoxy-3-(1-methyl-1H-1,2,4-triazol-3-yl)phenyl)amino)-6-(methylcarbamoyl)pyridazin-3-yl)amino)pyridin-3-yl)methyl)heptanediamide (CPD-130)

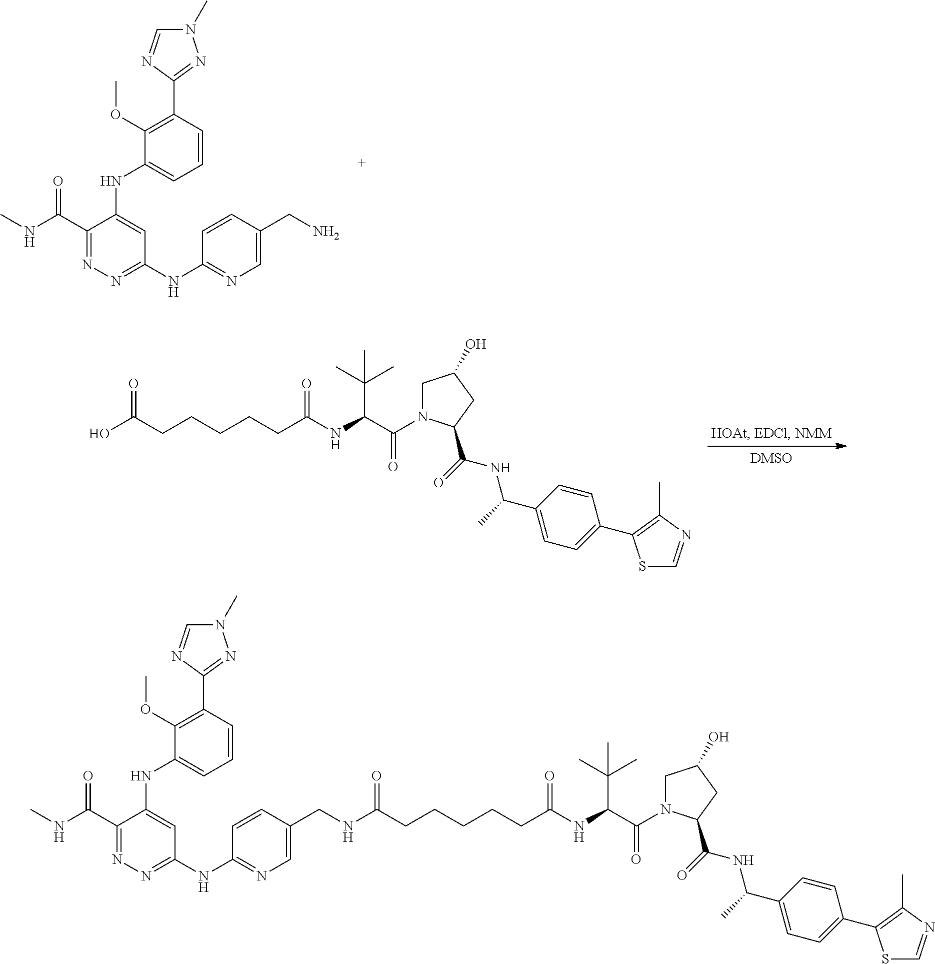 [SMILES: CNC(=O)c1nnc(Nc2ccc(CN)cn2)cc1Nc1cccc(-c2ncn(C)n2)c1OC ; Cc1ncsc1-c1ccc([C@H](C)NC(=O)[C@@H]2C[C@@H](O)CN2C(=O)[C@@H](NC(=O)CCCCCC(=O)O)C(C)(C)C)cc1 ; CNC(=O)c1nnc(Nc2ccc(CNC(=O)CCCCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC]

CPD-130 was synthesized following the same procedure for preparing CPD-128 (5.6 mg, yield: 31%). MS (ESI) m/z=1029.9 [M+H]⁺.

### Example 193: N1-((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)-N8-((6-((5-((2-methoxy-3-(1-methyl-1H-1,2,4-triazol-3-yl)phenyl)amino)-6-(methylcarbamoyl)pyridazin-3-yl)amino)pyridin-3-yl)methyl)octanediamide (CPD-131)

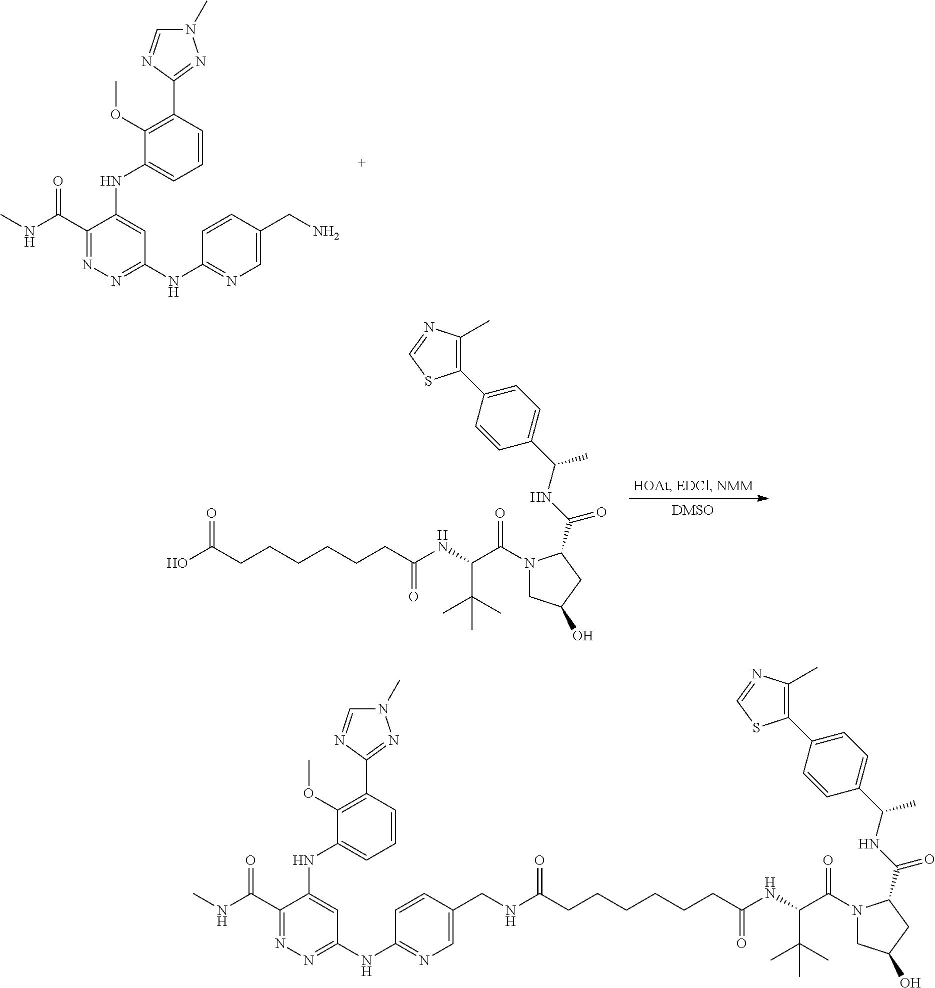 [SMILES: CNC(=O)c1nnc(Nc2ccc(CN)cn2)cc1Nc1cccc(-c2ncn(C)n2)c1OC ; Cc1ncsc1-c1ccc([C@H](C)NC(=O)[C@@H]2C[C@@H](O)CN2C(=O)[C@@H](NC(=O)CCCCCCC(=O)O)C(C)(C)C)cc1 ; CNC(=O)c1nnc(Nc2ccc(CNC(=O)CCCCCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC]

CPD-131 was synthesized following the same procedure for preparing CPD-128 (8 mg, yield: 51% O). MS (ESI) m/z=1044.1 [M+H]⁺.

### Example 194: N1-((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)-N9-((6-((5-((2-methoxy-3-(1-methyl-1H-1,2,4-triazol-3-yl)phenyl)amino)-6-(methylcarbamoyl)pyridazin-3-yl)amino)pyridin-3-yl)methyl)nonanediamide (CPD-132)

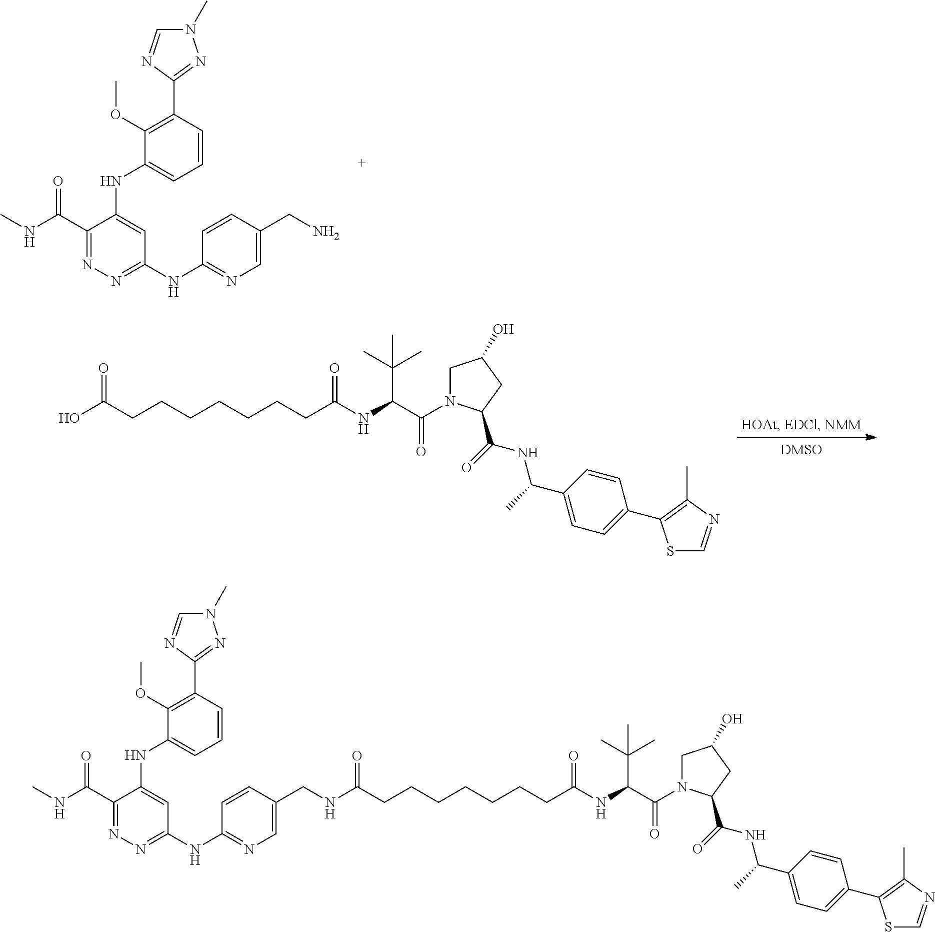 [SMILES: CNC(=O)c1nnc(Nc2ccc(CN)cn2)cc1Nc1cccc(-c2ncn(C)n2)c1OC ; Cc1ncsc1-c1ccc([C@H](C)NC(=O)[C@@H]2C[C@@H](O)CN2C(=O)[C@@H](NC(=O)CCCCCCCC(=O)O)C(C)(C)C)cc1 ; CNC(=O)c1nnc(Nc2ccc(CNC(=O)CCCCCCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC]

CPD-132 was synthesized following the same procedure for preparing CPD-128 (5.2 mg, yield: 29%). MS (ESI) m/z=1058.2 [M+H]⁺.

### Example 195: N1-((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)-N10-((6-((5-((2-methoxy-3-(1-methyl-1H-1,2,4-triazol-3-yl)phenyl)amino)-6-(methylcarbamoyl)pyridazin-3-yl)amino)pyridin-3-yl)methyl)decanediamide (CPD-133)

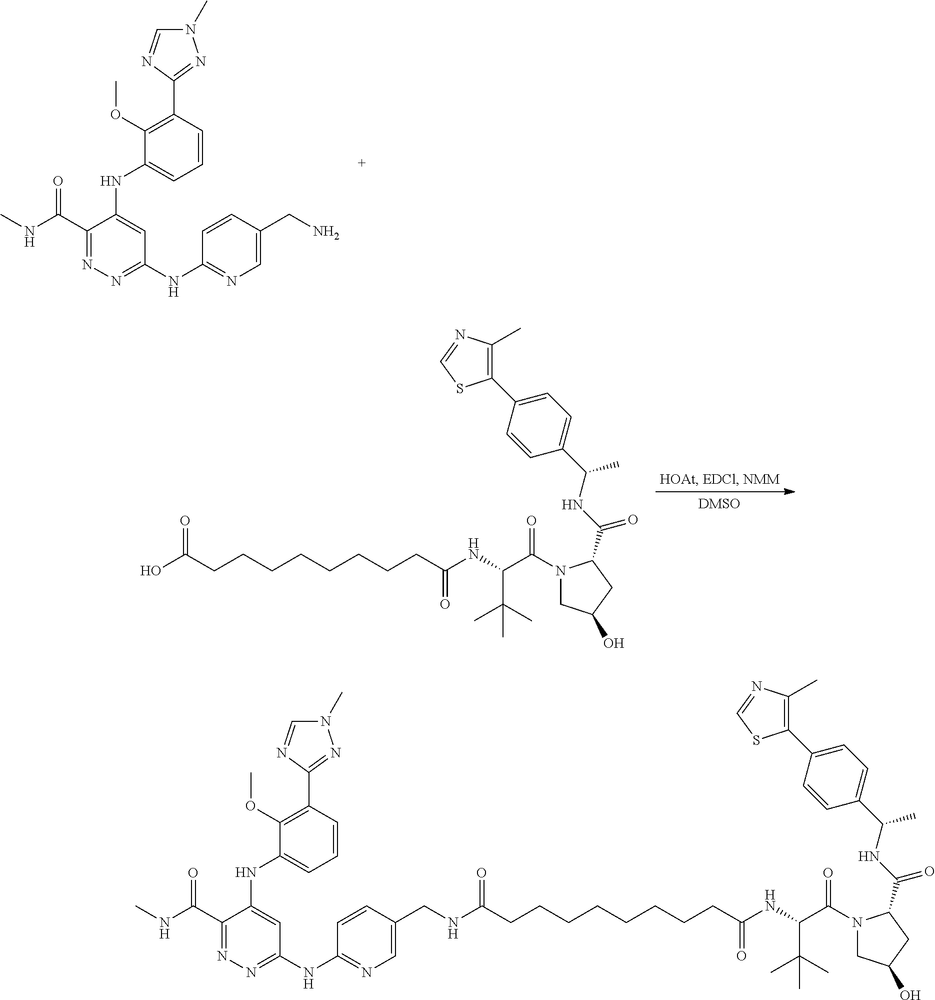 [SMILES: CNC(=O)c1nnc(Nc2ccc(CN)cn2)cc1Nc1cccc(-c2ncn(C)n2)c1OC ; Cc1ncsc1-c1ccc([C@H](C)NC(=O)[C@@H]2C[C@@H](O)CN2C(=O)[C@@H](NC(=O)CCCCCCCCC(=O)O)C(C)(C)C)cc1 ; CNC(=O)c1nnc(Nc2ccc(CNC(=O)CCCCCCCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC]

CPD-133 was synthesized following the same procedure for preparing CPD-128 (5.2 mg, yield: 29%). MS (ESI) m/z=1072.2 [M+H]⁺.

### Example 196: 6-((5-((1-(5-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentanoyl)piperidin-4-yl)ethynyl)pyridin-2-yl)amino)-4-((2-methoxy-3-(1-methyl-1H-1,2,4-triazol-3-yl)phenyl)amino)-N-methylpyridazine-3-carboxamide CPD-134

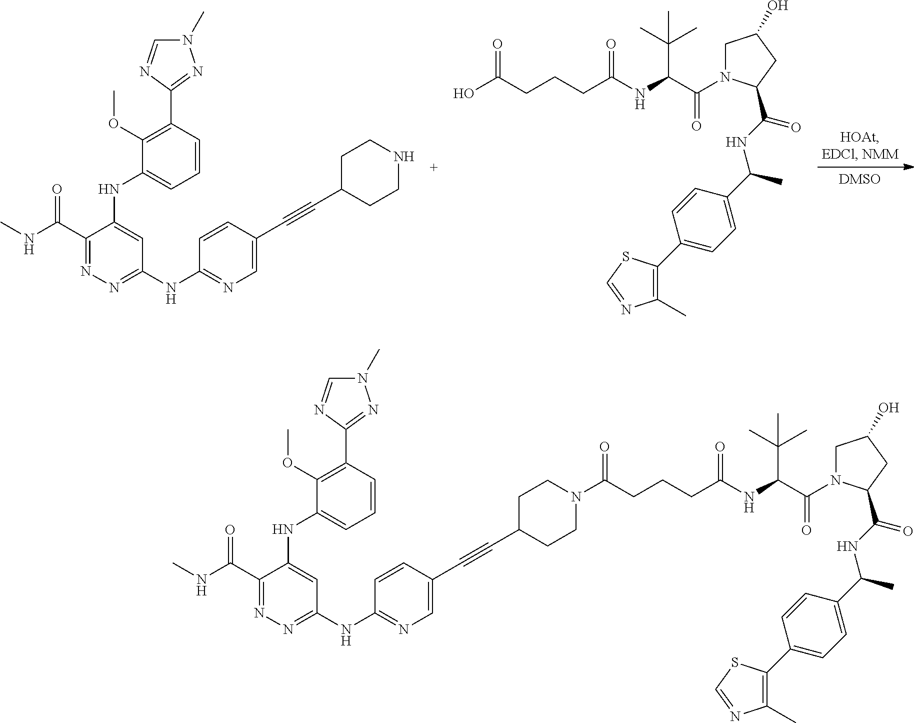 [SMILES: CNC(=O)c1nnc(Nc2ccc(C#CC3CCNCC3)cn2)cc1Nc1cccc(-c2ncn(C)n2)c1OC ; Cc1ncsc1-c1ccc([C@H](C)NC(=O)[C@@H]2C[C@@H](O)CN2C(=O)[C@@H](NC(=O)CCCC(=O)O)C(C)(C)C)cc1 ; CNC(=O)c1nnc(Nc2ccc(C#CC3CCN(C(=O)CCCC(=O)N[C@H](C(=O)N4C[C@H](O)C[C@H]4C(=O)N[C@@H](C)c4ccc(-c5scnc5C)cc4)C(C)(C)C)CC3)cn2)cc1Nc1cccc(-c2ncn(C)n2)c1OC]

CPD-134 was synthesized following the same procedure for preparing CPD-116 (12 mg, yield: 62%). MS (ESI) m/z=1080.0 [M+H]⁺.

### Example 197: 6-((5-((1-(5-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentanoyl)azetidin-3-yl)ethynyl)pyridin-2-yl)amino)-4-((2-methoxy-3-(1-methyl-1H-1,2,4-triazol-3-yl)phenyl)amino)-N-methylpyridazine-3-carboxamide (CPD-135)

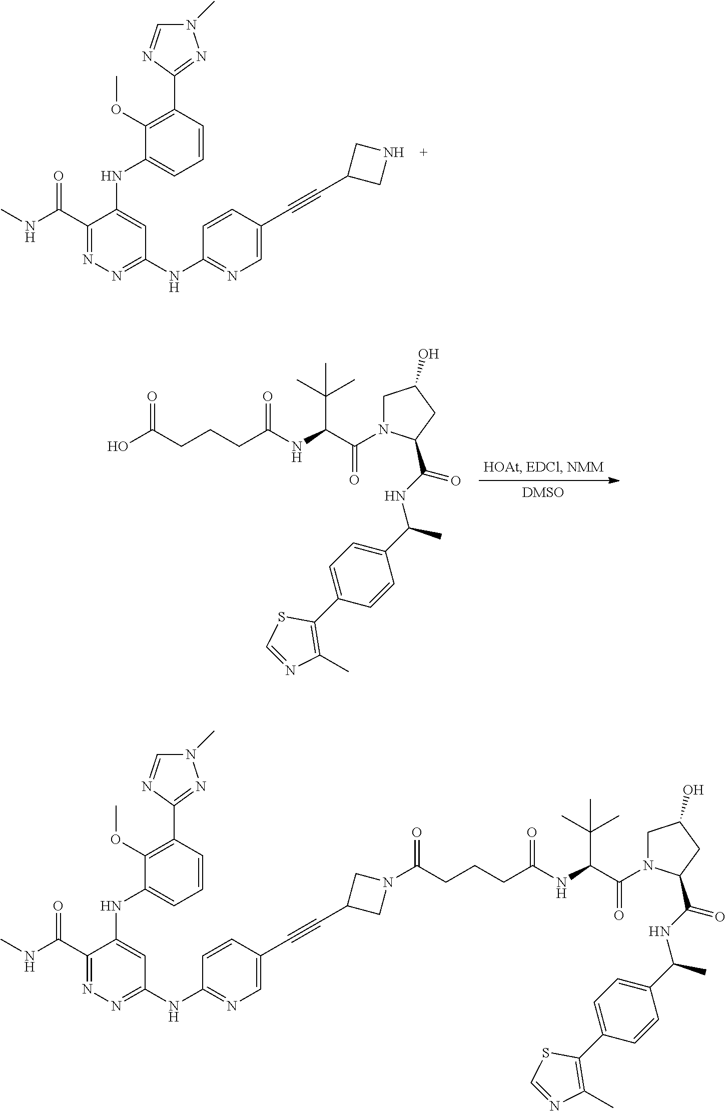 [SMILES: CNC(=O)c1nnc(Nc2ccc(C#CC3CNC3)cn2)cc1Nc1cccc(-c2ncn(C)n2)c1OC ; Cc1ncsc1-c1ccc([C@H](C)NC(=O)[C@@H]2C[C@@H](O)CN2C(=O)[C@@H](NC(=O)CCCC(=O)O)C(C)(C)C)cc1 ; CNC(=O)c1nnc(Nc2ccc(C#CC3CN(C(=O)CCCC(=O)N[C@H](C(=O)N4C[C@H](O)C[C@H]4C(=O)N[C@@H](C)c4ccc(-c5scnc5C)cc4)C(C)(C)C)C3)cn2)cc1Nc1cccc(-c2ncn(C)n2)c1OC]

CPD-135 was synthesized following the same procedure for preparing CPD-116 (10 mg, yield: 53.2%). MS (ESI) m/z=1051.8 [M+H]⁺.

### Example 198: N1-((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)-N5-((6-((5-((2-methoxy-3-(1-methyl-1H-1,2,4-triazol-3-yl)phenyl)amino)-6-(methylcarbamoyl)pyridazin-3-yl)amino)pyridin-3-yl)methyl)glutaramide (CPD-136)

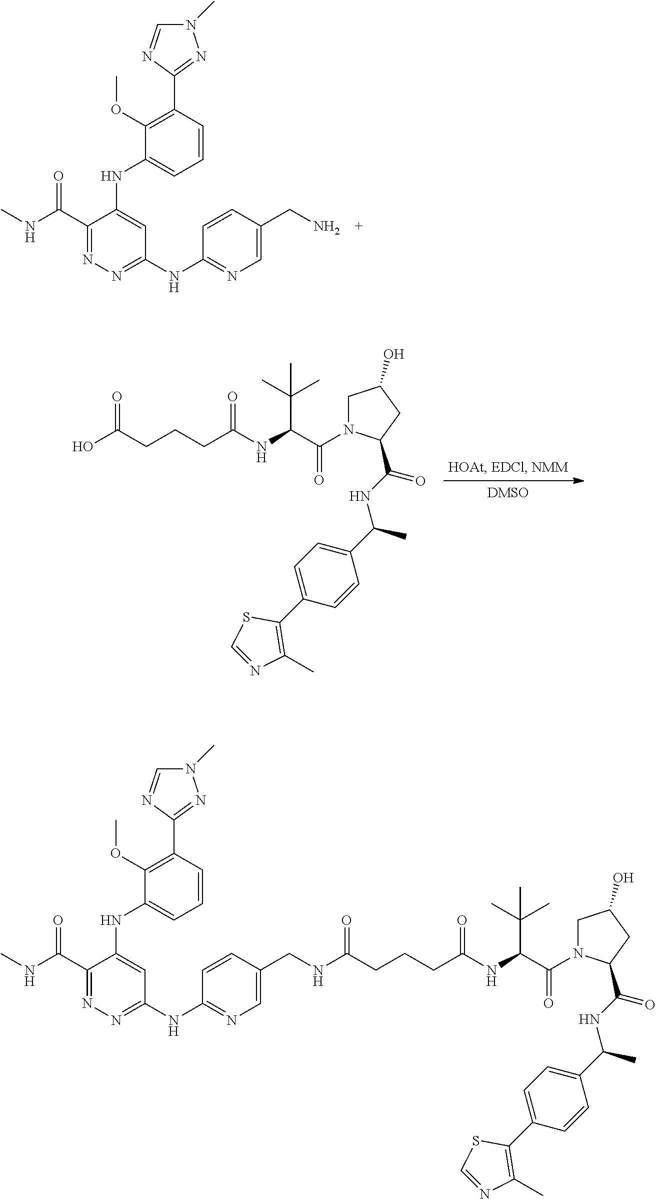 [SMILES: CNC(=O)c1nnc(Nc2ccc(CN)cn2)cc1Nc1cccc(-c2ncn(C)n2)c1OC ; Cc1ncsc1-c1ccc([C@H](C)NC(=O)[C@@H]2C[C@@H](O)CN2C(=O)[C@@H](NC(=O)CCCC(=O)O)C(C)(C)C)cc1 ; CNC(=O)c1nnc(Nc2ccc(CNC(=O)CCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC]

CPD-136 was synthesized following the same procedure for preparing CPD-128 (12 mg, yield: 67%). MS (ESI) m/z=1001.8 [M+H]⁺.

### Example 199: 6-((5-((2-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-137)

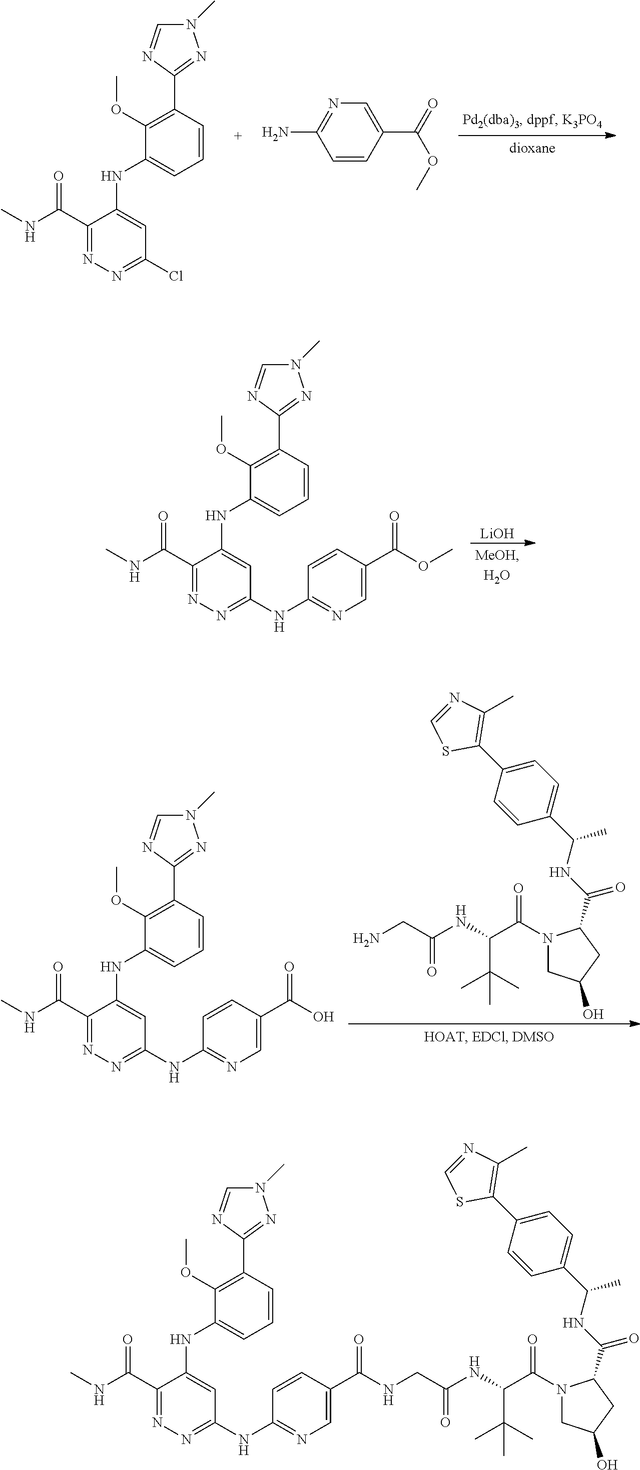

### Step 1. Synthesis of methyl 6-((5-((2-methoxy-3-(1-methyl-1H-1,2,4-triazol-3-yl)phenyl)amino)-6-(methylcarbamoyl)pyridazin-3-yl)amino)nicotinate

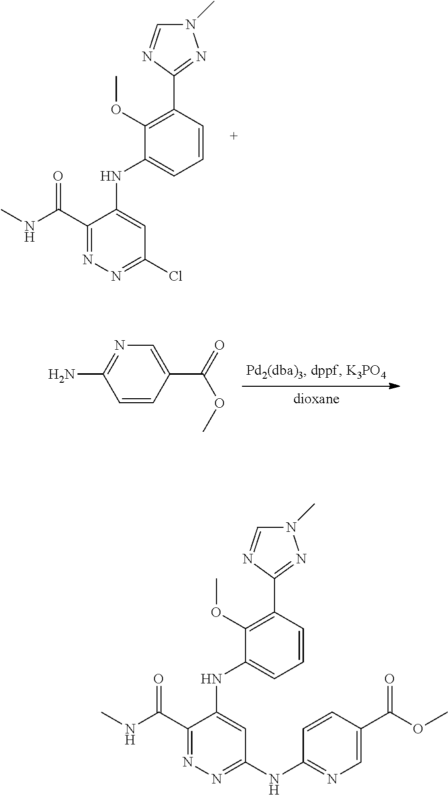

To a mixture of 6-chloro-4-((2-methoxy-3-(1-methyl-1H-1,2,4-triazol-3-yl)phenyl)amino)-N-methylpyridazine-3-carboxamide (200 mg, 0.54 mmol) and K₃PO₄ (343 mg, 1.62 mmol) in dioxane (5 mL) were added methyl 6-aminonicotinate (99 mg, 0.65 mmol), Pd₂(dba)₃ (10.7 mg, 0.011 mmol) and dppf (10 mg, 0.022 mmol). After the resulting mixture was stirred at 100° C. for 16 h, it was diluted with water and extracted with EtOAc (20 mL×3). The combined organic layers were washed with brine (20 mL×2), dried over Na₂SO₄, filtered, and concentrated under reduced pressure. The residue was purified by reverse-phase chromatography to give the title compound (187 mg, yield: 71.4%) as a light yellow solid. MS (ESI) m/z=490.3 [M+H]⁺.

### Step 2. Synthesis of 6-((5-((2-methoxy-3-(1-methyl-1H-1,2,4-triazol-3-yl)phenyl)amino)-6-(methylcarbamoyl)pyridazin-3-yl)amino)nicotinic acid

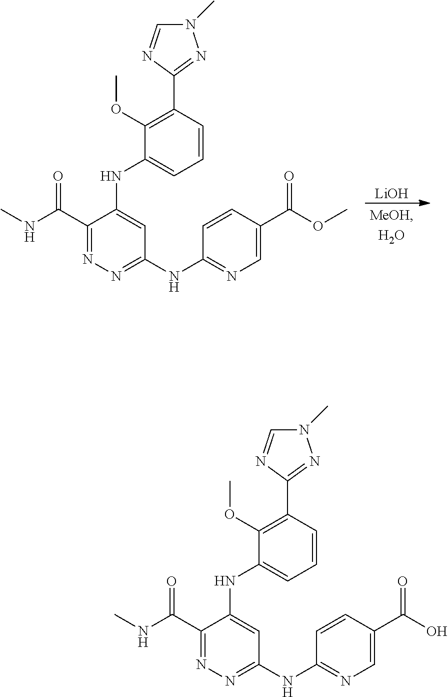

To a mixture of methyl 6-((5-((2-methoxy-3-(1-methyl-1H-1,2,4-triazol-3-yl)phenyl)amino)-6-(methylcarbamoyl)pyridazin-3-yl)amino)nicotinate (187 mg, 0.38 mmol) in MeOH (20 mL) were added H₂O (2 mL) and LiOH (91 mg, 3.8 mmol). After the resulting mixture was stirred at 25° C. for 48 h, it was concentrated under reduced pressure. The residue was purified by reverse-phase chromatography to give the title compound (131 mg, yield: 72.4%) as a light-yellow solid. MS (ESI) m/z=476.4 [M+H]⁺.

### Step 3. Synthesis of 6-((5-((2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)carbamoyl)pyridin-2-yl)amino)-4-((2-methoxy-3-(1-methyl-1H-1,2,4-triazol-3-yl)phenyl)amino)-N-methylpyridazine-3-carboxamide

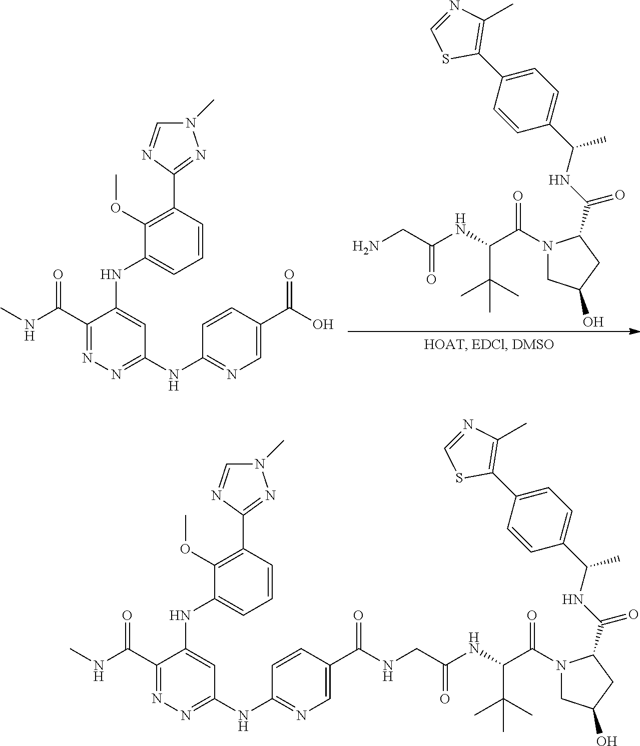 [SMILES: CNC(=O)c1nnc(Nc2ccc(C(=O)O)cn2)cc1Nc1cccc(-c2ncn(C)n2)c1OC ; Cc1ncsc1-c1ccc([C@H](C)NC(=O)[C@@H]2C[C@@H](O)CN2C(=O)[C@@H](NC(=O)CN)C(C)(C)C)cc1 ; CNC(=O)c1nnc(Nc2ccc(C(=O)NCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC]

CPD-137 was synthesized following the same procedure for preparing CPD-042 (6.3 mg, yield: 66.3%). MS (ESI) m/z=959.8 [M+H]⁺.

### Example 200: 6-((5-((3-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-138)

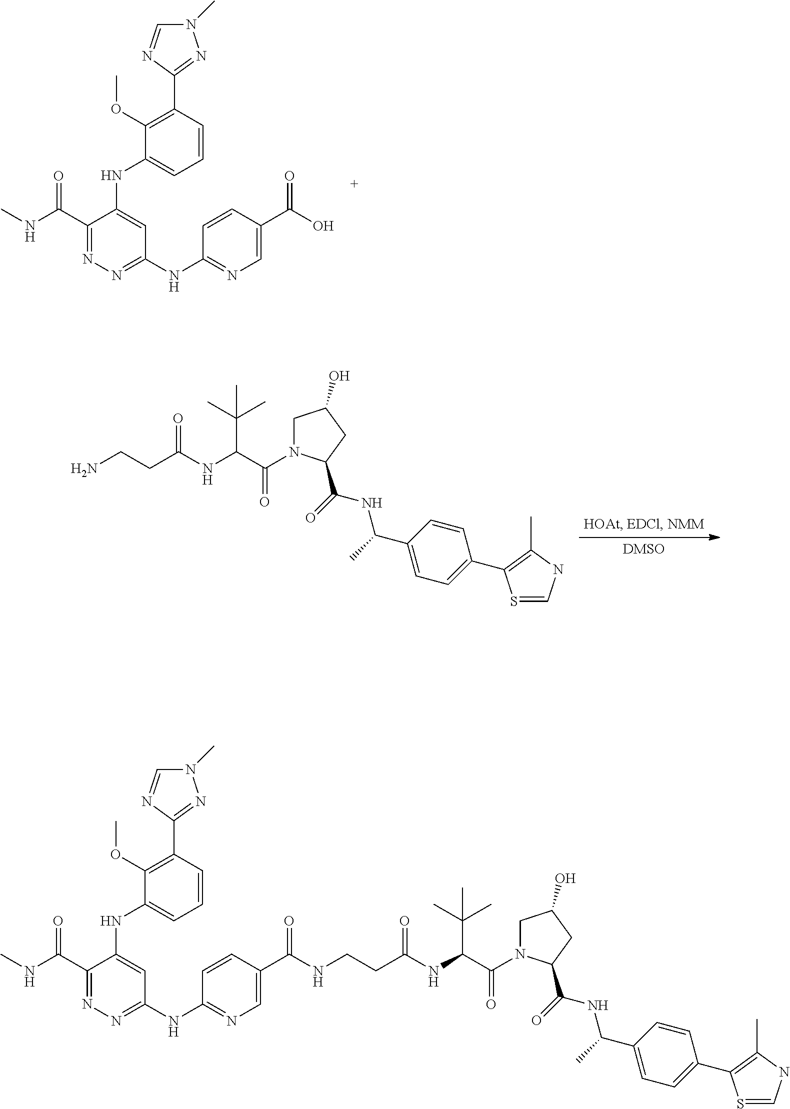

CPD-138 was synthesized following the same procedure for preparing CPD-137 (6.3 mg, yield: 64.6%). MS (ESI) m/z=973.8 [M+H]⁺.

### Example 201: 6-((5-((4-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-139)

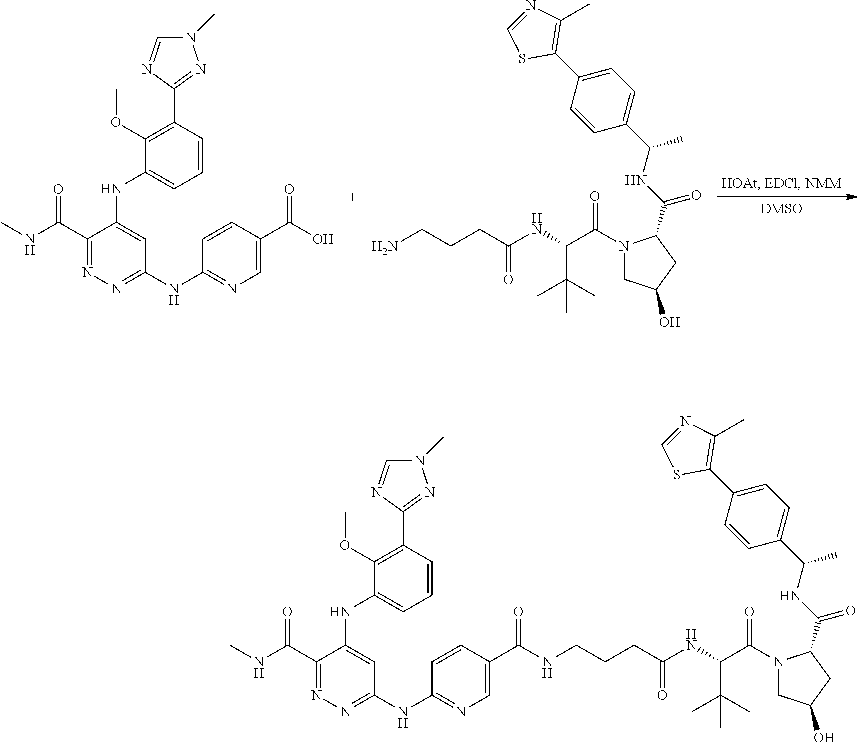 [SMILES: CNC(=O)c1nnc(Nc2ccc(C(=O)O)cn2)cc1Nc1cccc(-c2ncn(C)n2)c1OC ; Cc1ncsc1-c1ccc([C@H](C)NC(=O)[C@@H]2C[C@@H](O)CN2C(=O)[C@@H](NC(=O)CCCN)C(C)(C)C)cc1 ; CNC(=O)c1nnc(Nc2ccc(C(=O)NCCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC]

CPD-139 was synthesized following the same procedure for preparing CPD-137 (12 mg, yield: 67%). MS (ESI) m/z=987.6 [M+H]⁺.

### Example 202: 6-((5-((5-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-140)

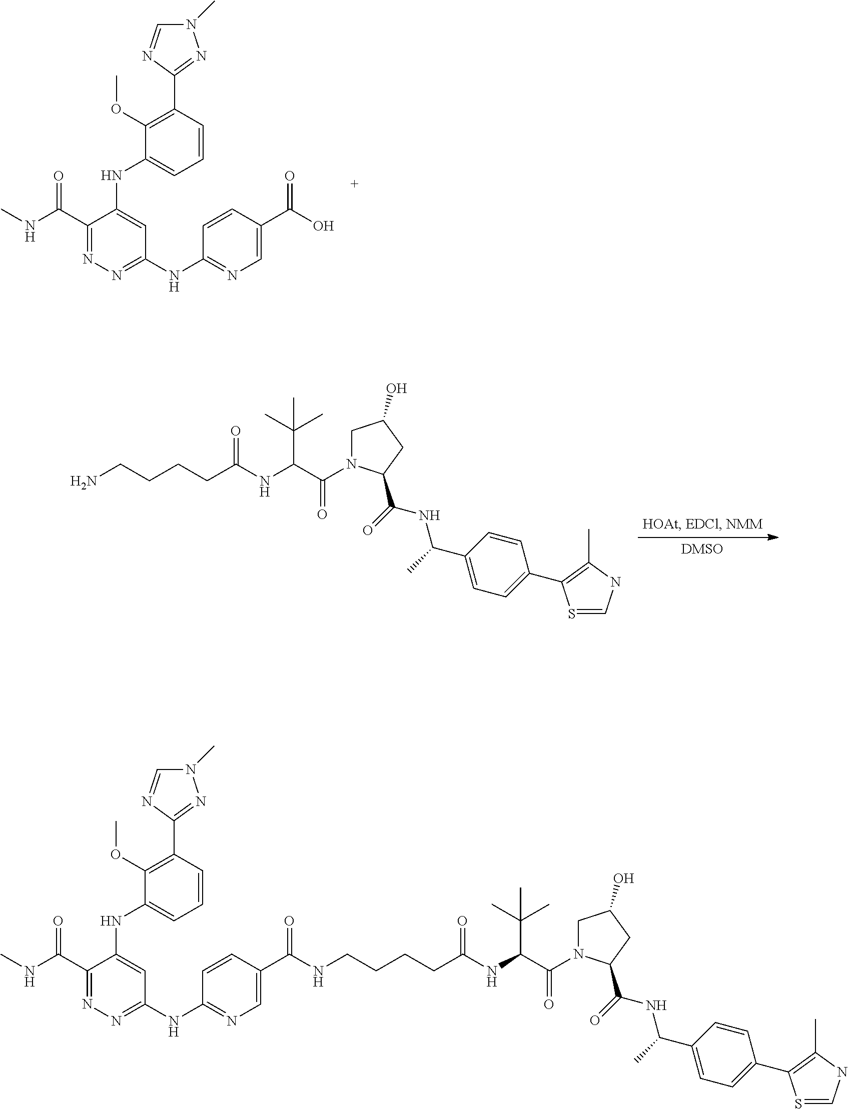

CPD-140 was synthesized following the same procedure for preparing CPD-137 (3 mg, yield: 32.6%). MS (ESI) m/z=1001.9 [M+H]⁺.

### Example 203: 6-((5-((6-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-141)

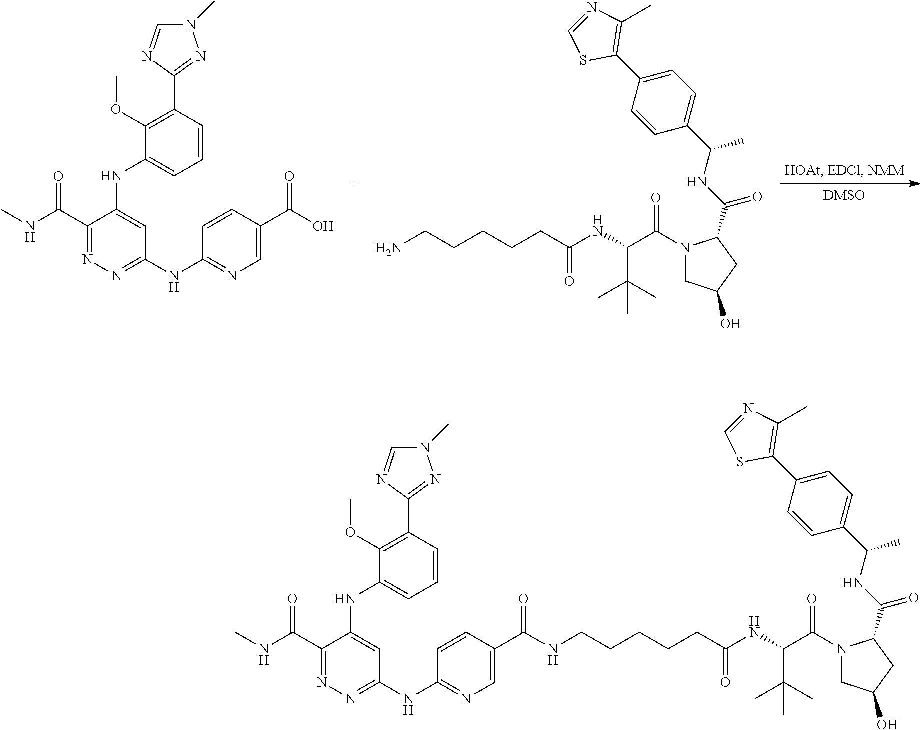 [SMILES: CNC(=O)c1nnc(Nc2ccc(C(=O)O)cn2)cc1Nc1cccc(-c2ncn(C)n2)c1OC ; Cc1ncsc1-c1ccc([C@H](C)NC(=O)[C@@H]2C[C@@H](O)CN2C(=O)[C@@H](NC(=O)CCCCCN)C(C)(C)C)cc1 ; CNC(=O)c1nnc(Nc2ccc(C(=O)NCCCCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC]

CPD-141 was synthesized following the same procedure for preparing CPD-137 (5.3 mg, yield: 47.3%). MS (ESI) m/z=1015.7 [M+H]⁺.

### Example 204: 6-((5-((7-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptyl)carbamoyl)pyridin-2-yl)amino)-4-((2-methoxy-3-(1-methyl-1H-1,2,4-triazol-3-yl)phenyl)amino)-N-methylpridazine-3-carboxamide (CPD-142)

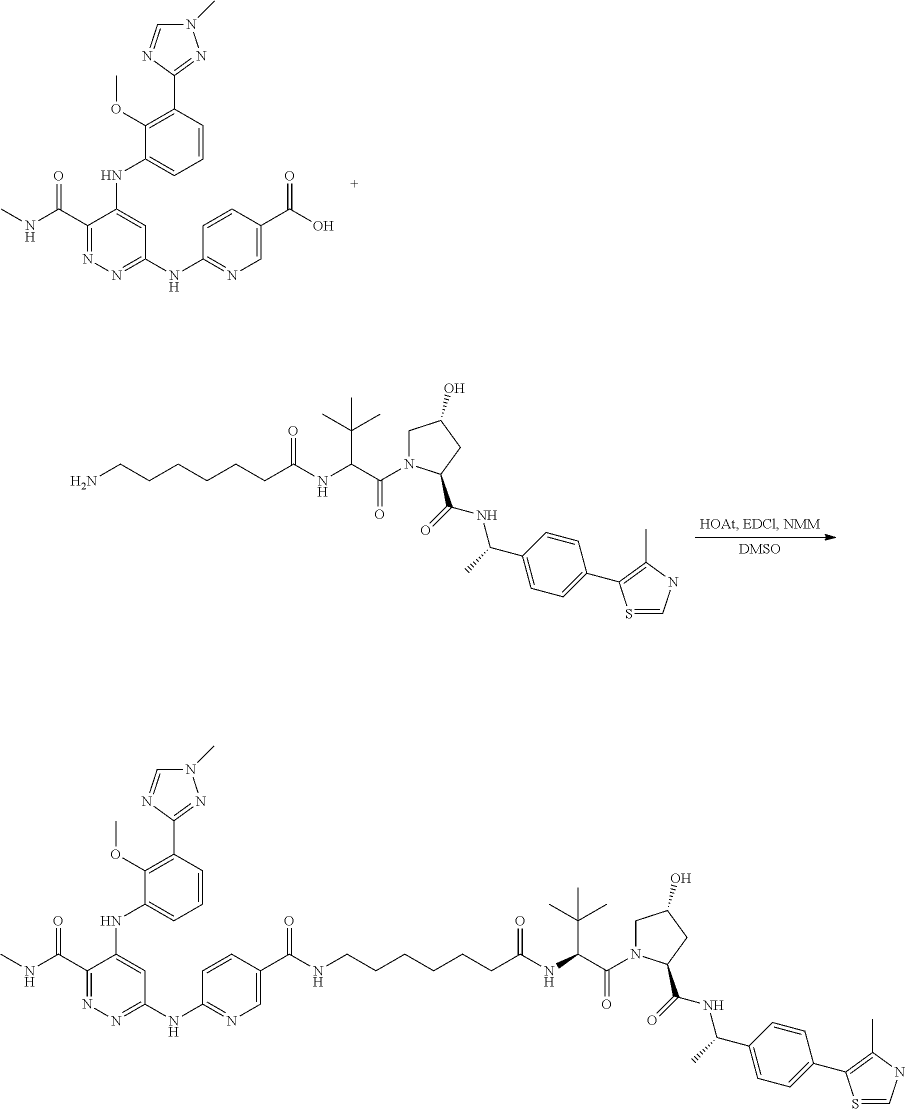

CPD-142 was synthesized following the same procedure for preparing CPD-137 (3.8 mg, yield: 27.1%). MS (ESI) m/z=1029.9 [M+H]⁺.

### Example 205: 6-((5-((8-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-143)

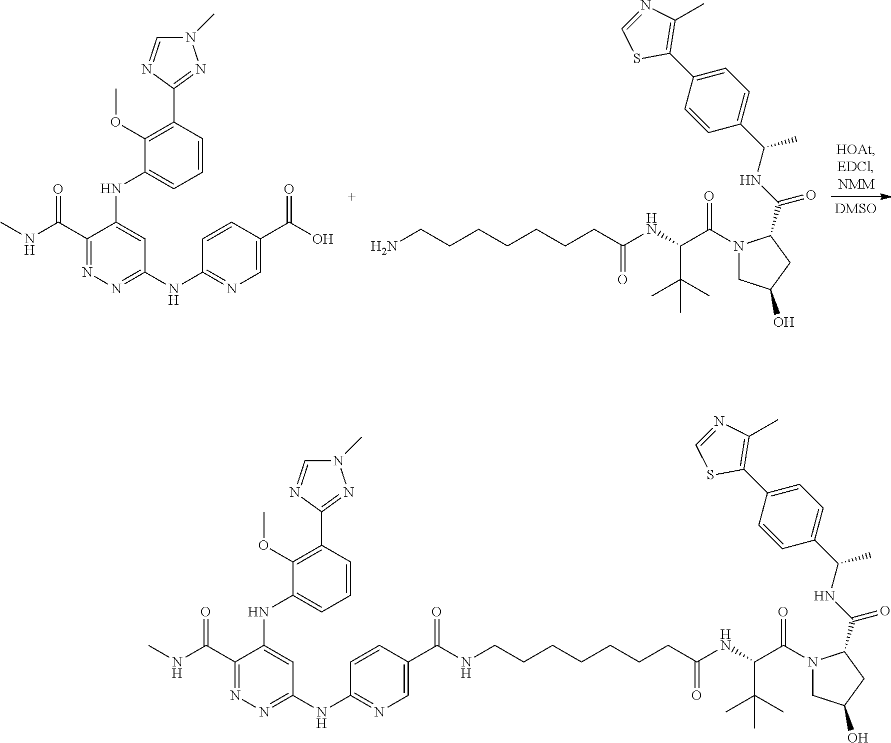 [SMILES: CNC(=O)c1nnc(Nc2ccc(C(=O)O)cn2)cc1Nc1cccc(-c2ncn(C)n2)c1OC ; Cc1ncsc1-c1ccc([C@H](C)NC(=O)[C@@H]2C[C@@H](O)CN2C(=O)[C@@H](NC(=O)CCCCCCCN)C(C)(C)C)cc1 ; CNC(=O)c1nnc(Nc2ccc(C(=O)NCCCCCCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC]

CPD-143 was synthesized following the same procedure for preparing CPD-137 (5.6 mg, yield: 40%). MS (ESI) m/z=1044.1 [M+H]⁺.

### Example 206: 6-((5-((9-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-144)

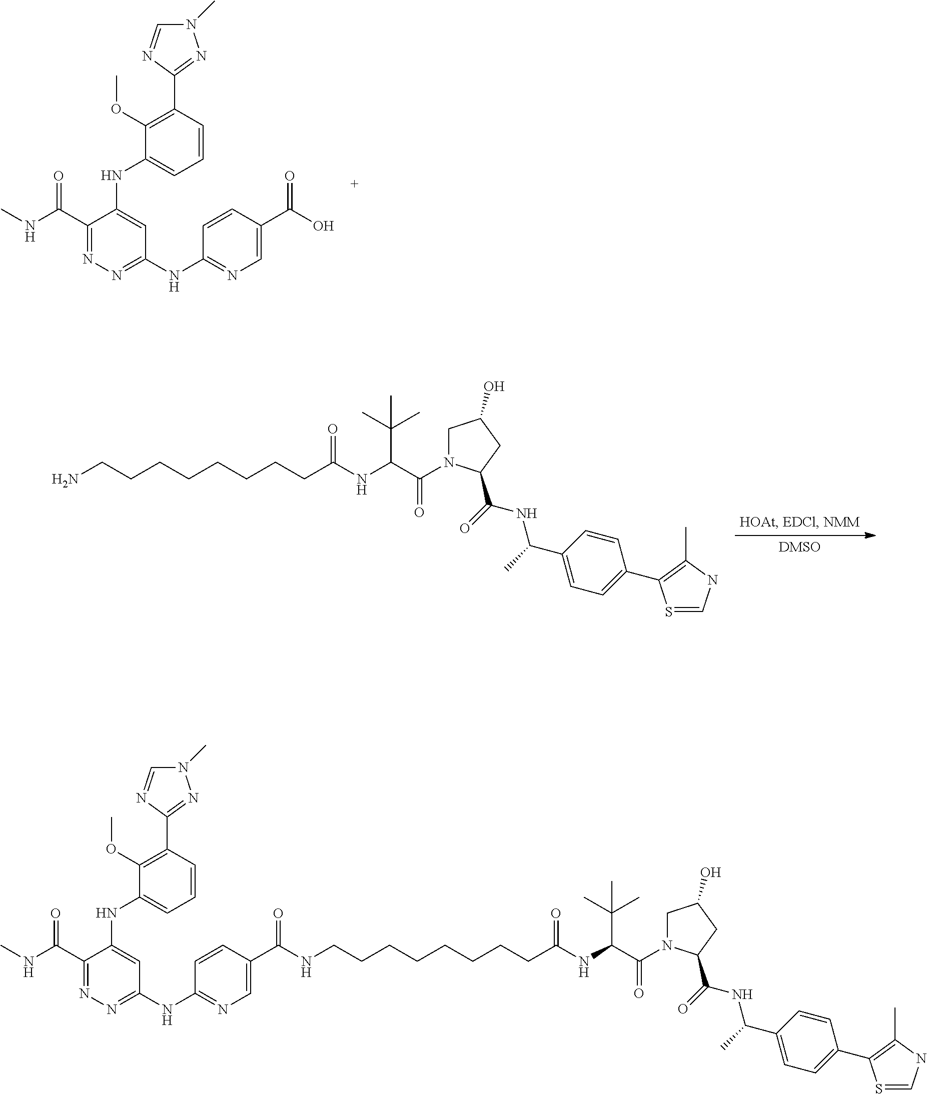

CPD-144 was synthesized following the same procedure for preparing CPD-137 (7.3 mg, yield: 68.9%). MS (ESI) m/z=1058.0 [M+H]⁺.

### Example 207: 6-((5-(4-(1-(4-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutanoyl)piperidin-4-yl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-145)

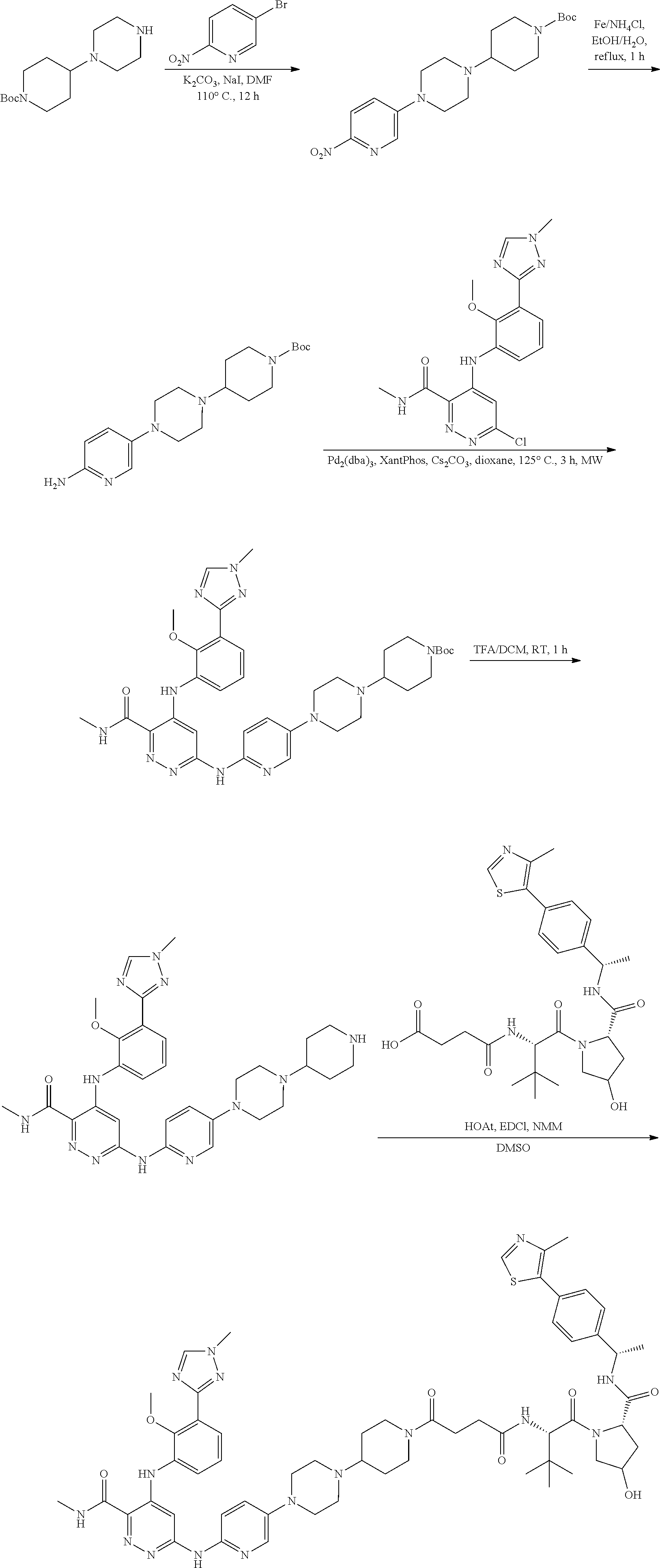

### Step 1. Synthesis of tert-butyl 4-(4-(6-nitropyridin-3-yl)piperazin-1-yl)piperidine-1-carboxylate

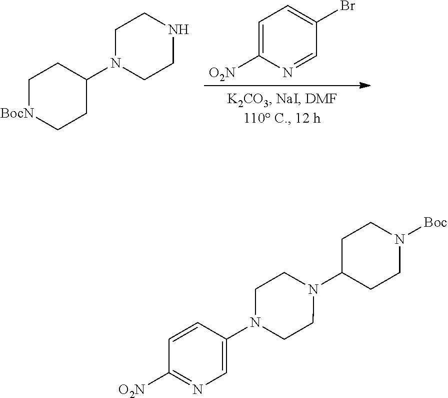 [SMILES: CC(C)(C)OC(=O)N1CCC(N2CCNCC2)CC1 ; O=[N+]([O-])c1ccc(Br)cn1 ; CC(C)(C)OC(=O)N1CCC(N2CCN(c3ccc([N+](=O)[O-])nc3)CC2)CC1]

To a suspension of tert-butyl 4-(piperazin-1-yl)piperidine-1-carboxylate (250.0 mg, 0.93 mmol), K₂CO₃ (231.0 mg, 1.67 mmol) and NaI (143.2 mg, 0.93 mmol) in DMF (4 mL) was added 5-bromo-2-nitropyridine (282.6 mg, 1.39 mmol). After the reaction mixture was stirred at 110° C. for 12 h, it was cooled to rt and filtered through celite. The filtrate was washed with water and extracted with EtOAc. The organic layer was concentrated, and the residue was purified by column chromatography on silica gel (petroleum ether/EtOAc=1:1) to give the title compound (260 mg, 71.4% yield) as a yellow solid.

### Step 2. Synthesis of tert-butyl 4-(4-(6-aminopyridin-3-yl)piperazin-1-yl)piperidine-1-carboxylate

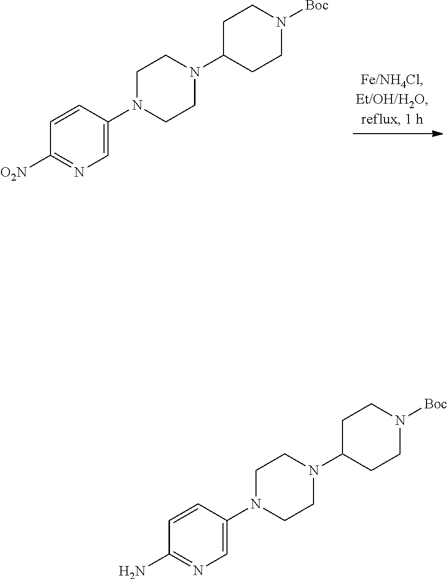 [SMILES: CC(C)(C)OC(=O)N1CCC(N2CCN(c3ccc([N+](=O)[O-])nc3)CC2)CC1 ; CC(C)(C)OC(=O)N1CCC(N2CCN(c3ccc(N)nc3)CC2)CC1]

To a suspension of tert-butyl 4-(4-(6-nitropyridin-3-yl)piperazin-1-yl)piperidine-1-carboxylate (260.0 mg, 0.66 mmol) and NH₄Cl (285.3 mg, 5.31 mmol) in EtOH (8 mL) and H₂O (1 mL) was added iron powder (148.4 mg, 2.65 mmol). After the reaction mixture was stirred at reflux for 1 h, it was cooled to rt and filtered through celite. The filtrate was concentrated, and the residue was purified by reverse-phase chromatography and re-purified by silica gel column chromatography (DCM/MeOH=8:1) to give the title compound (162.0 mg, 67.9% yield) as a white solid. MS (ESI) m/z=362.3 [M+H]⁺.

### Step 3. Synthesis of tert-butyl 4-(4-(6-((5-((2-methoxy-3-(1-methyl-1H-1,2,4-triazol-3-yl)phenyl)amino)-6-(methylcarbamoyl)pyridazin-3-yl)amino)pyridin-3-yl)piperazin-1-yl)piperidine-1-carboxylate

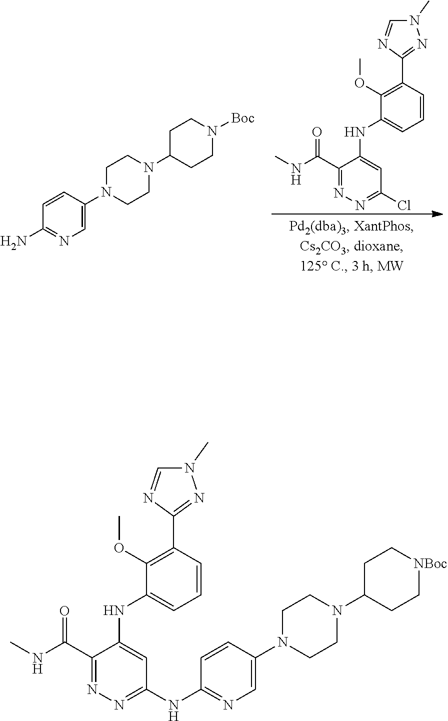

To a suspension of tert-butyl 4-(4-(6-aminopyridin-3-yl)piperazin-1-yl)piperidine-1-carboxylate (123.0 mg, 0.34 mmol), 6-chloro-4-((2-methoxy-3-(1-methyl-1H-1,2,4-triazol-3-yl)phenyl)amino)-N-methylpyridazine-3-carboxamide (152.6 mg, 0.41 mmol), XantPhos (98.4 mg, 0.17 mmol) and Cs₂CO₃ (332.5 mg, 1.02 mmol) in dioxane (1.5 mL) was added Pd₂(dba)₃ (36.6 mg, 0.04 mmol). The reaction mixture was degassed and stirred under microwave irradiation at 125° C. for 3 h. After cooling down to rt, the solution was concentrated under reduced pressure. The residue was purified by reverse-phase chromatography to give the title compound (150 mg, 63.1% yield) as a yellow solid. MS (ESI) m/z=699.7 [M+H]⁺.

### Step 4. Synthesis of 4-((2-methoxy-3-(1-methyl-1H-1,2,4-triazol-3-yl)phenyl)amino)-N-methyl-6-((5-(4-(piperidin-4-yl)piperazin-1-yl)pyridin-2-yl)amino)pyridazine-3-carboxamide

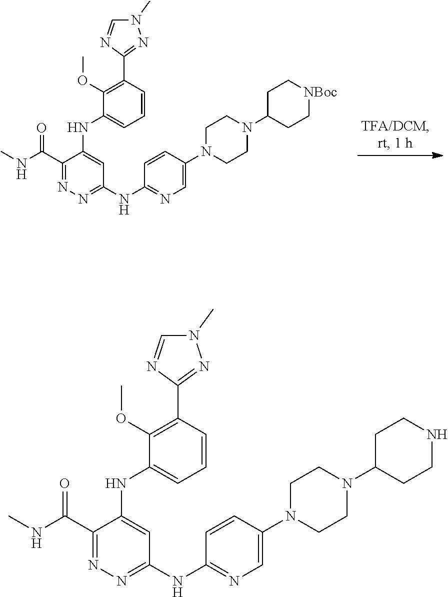 [SMILES: CNC(=O)c1nnc(Nc2ccc(N3CCN(C4CCN(C(=O)OC(C)(C)C)CC4)CC3)cn2)cc1Nc1cccc(-c2ncn(C)n2)c1OC ; CNC(=O)c1nnc(Nc2ccc(N3CCN(C4CCNCC4)CC3)cn2)cc1Nc1cccc(-c2ncn(C)n2)c1OC]

A solution of tert-butyl 4-(4-(6-((5-((2-methoxy-3-(1-methyl-1H-1,2,4-triazol-3-yl)phenyl) amino)-6-(methylcarbamoyl)pyridazin-3-yl)amino)pyridin-3-yl)piperazin-1-yl)piperidine-1-carboxylate (150 mg, 0.21 mmol) in TFA (1.0 mL) and DCM (1.0 mL) was stirred at room temperature for 1 h. The solution was concentrated under reduced pressure to give the crude product (120 mg 93.6% yield) as a yellow solid which was used directly in the next step without further purification. MS (ESI) m/z=599.5 [M+H]⁺.

### Step 5. Synthesis of 6-((5-(4-(1-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutanoyl)piperidin-4-yl)piperazin-1-yl)pyridin-2-yl)amino)-4-((2-methoxy-3-(1-methyl-1H-1,2,4-triazol-3-yl)phenyl)amino)-N-methylpyridazine-3-carboxamide

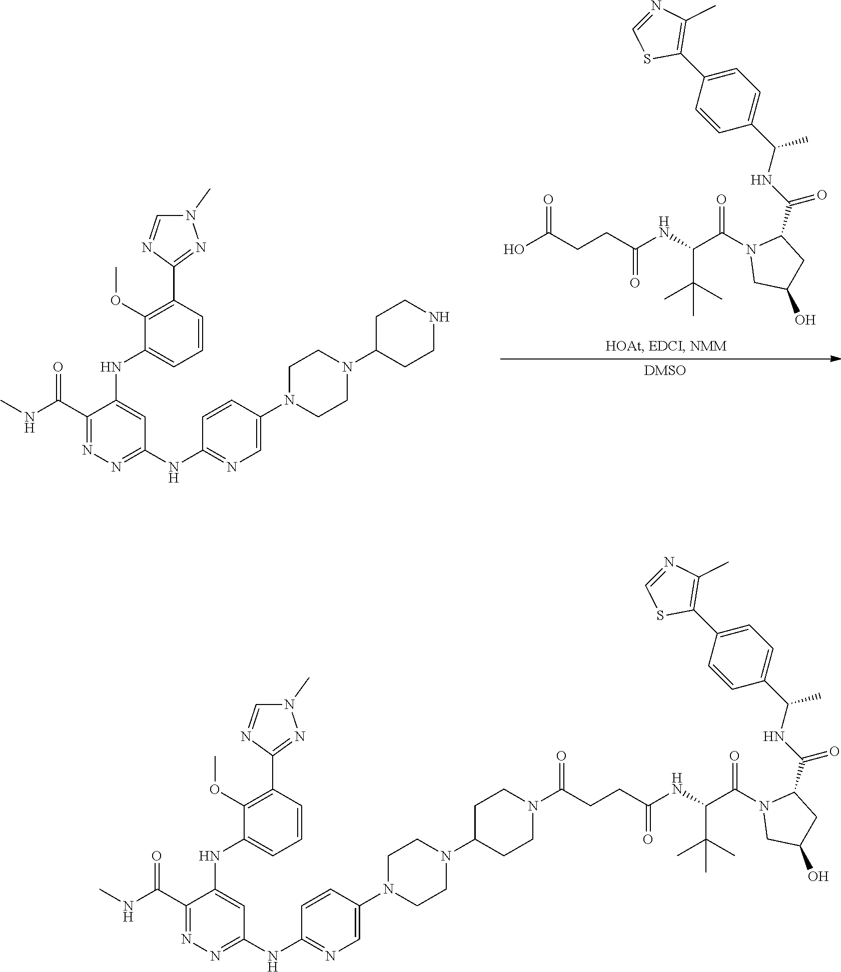 [SMILES: CNC(=O)c1nnc(Nc2ccc(N3CCN(C4CCNCC4)CC3)cn2)cc1Nc1cccc(-c2ncn(C)n2)c1OC ; Cc1ncsc1-c1ccc([C@H](C)NC(=O)[C@@H]2C[C@@H](O)CN2C(=O)[C@@H](NC(=O)CCC(=O)O)C(C)(C)C)cc1 ; CNC(=O)c1nnc(Nc2ccc(N3CCN(C4CCN(C(=O)CCC(=O)N[C@H](C(=O)N5C[C@H](O)C[C@H]5C(=O)N[C@@H](C)c5ccc(-c6scnc6C)cc5)C(C)(C)C)CC4)CC3)cn2)cc1Nc1cccc(-c2ncn(C)n2)c1OC]

CPD-145 was synthesized following the same procedure for preparing CPD-042 (2.6 mg, yield: 27.4%). MS (ESI) m/z=1126.0 [M+H]⁺.

### Example 208: 6-((5-(4-(1-(5-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentanoyl)piperidin-4-yl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-146)

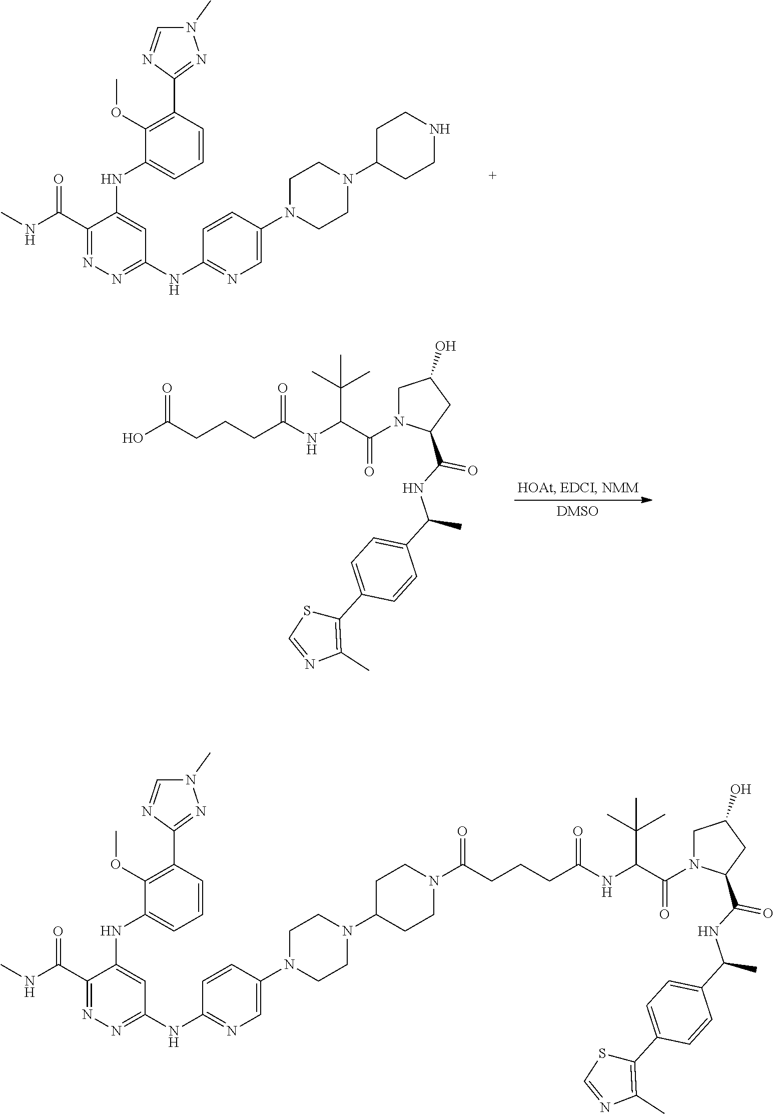 [SMILES: CNC(=O)c1nnc(Nc2ccc(N3CCN(C4CCNCC4)CC3)cn2)cc1Nc1cccc(-c2ncn(C)n2)c1OC ; Cc1ncsc1-c1ccc([C@H](C)NC(=O)[C@@H]2C[C@@H](O)CN2C(=O)C(NC(=O)CCCC(=O)O)C(C)(C)C)cc1 ; CNC(=O)c1nnc(Nc2ccc(N3CCN(C4CCN(C(=O)CCCC(=O)NC(C(=O)N5C[C@H](O)C[C@H]5C(=O)N[C@@H](C)c5ccc(-c6scnc6C)cc5)C(C)(C)C)CC4)CC3)cn2)cc1Nc1cccc(-c2ncn(C)n2)c1OC]

CPD-146 was synthesized following the same procedure for preparing CPD-145 (3.3 mg, yield: 34.7%). MS (ESI) m/z=1139.9 [M+H]⁺.

### Example 209: 6-((5-(4-(1-(6-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexanoyl)piperidin-4-yl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-147)

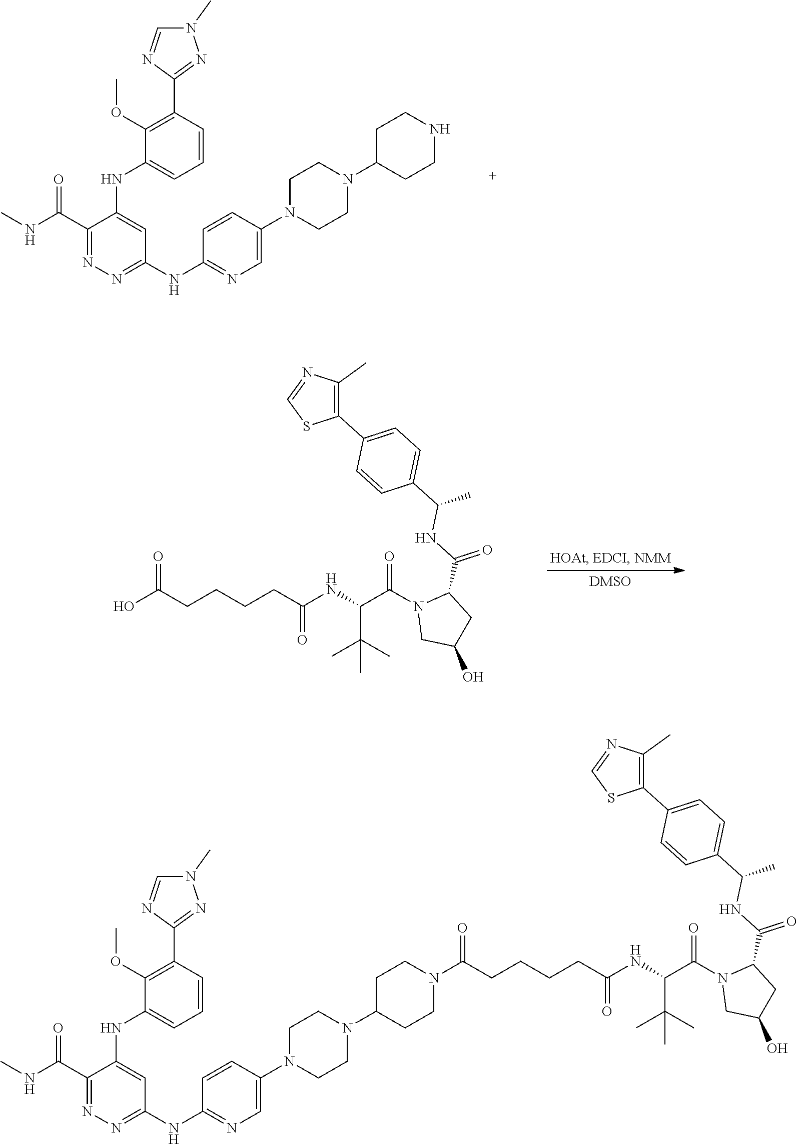 [SMILES: CNC(=O)c1nnc(Nc2ccc(N3CCN(C4CCNCC4)CC3)cn2)cc1Nc1cccc(-c2ncn(C)n2)c1OC ; Cc1ncsc1-c1ccc([C@H](C)NC(=O)[C@@H]2C[C@@H](O)CN2C(=O)[C@@H](NC(=O)CCCCC(=O)O)C(C)(C)C)cc1 ; CNC(=O)c1nnc(Nc2ccc(N3CCN(C4CCN(C(=O)CCCCC(=O)N[C@H](C(=O)N5C[C@H](O)C[C@H]5C(=O)N[C@@H](C)c5ccc(-c6scnc6C)cc5)C(C)(C)C)CC4)CC3)cn2)cc1Nc1cccc(-c2ncn(C)n2)c1OC]

CPD-147 was synthesized following the same procedure for preparing CPD-145 (3.4 mg, yield: 35%). MS (ESI) m/z=1154.2 [M+H]⁺.

### Example 210: 6-((5-(4-(1-(7-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoyl)piperidin-4-yl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-148)

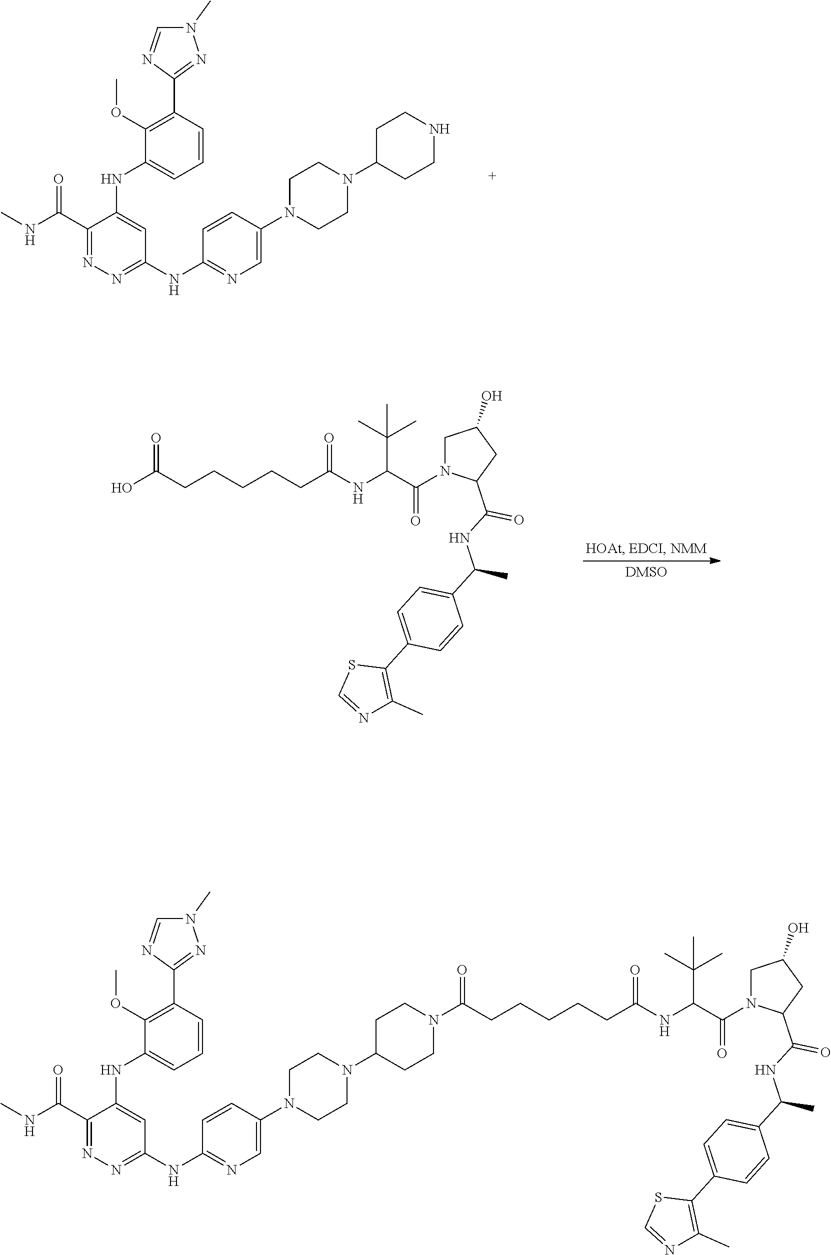 [SMILES: CNC(=O)c1nnc(Nc2ccc(N3CCN(C4CCNCC4)CC3)cn2)cc1Nc1cccc(-c2ncn(C)n2)c1OC ; Cc1ncsc1-c1ccc([C@H](C)NC(=O)C2C[C@@H](O)CN2C(=O)C(NC(=O)CCCCCC(=O)O)C(C)(C)C)cc1 ; CNC(=O)c1nnc(Nc2ccc(N3CCN(C4CCN(C(=O)CCCCCC(=O)NC(C(=O)N5C[C@H](O)CC5C(=O)N[C@@H](C)c5ccc(-c6scnc6C)cc5)C(C)(C)C)CC4)CC3)cn2)cc1Nc1cccc(-c2ncn(C)n2)c1OC]

CPD-148 was synthesized following the same procedure for preparing CPD-145 (3.4 mg, yield: 35.2%). MS (ESI) m/z=1169.1 [M+H]⁺.

### Example 211: 6-((5-(4-(1-(8-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctanoyl)piperidin-4-yl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-149)

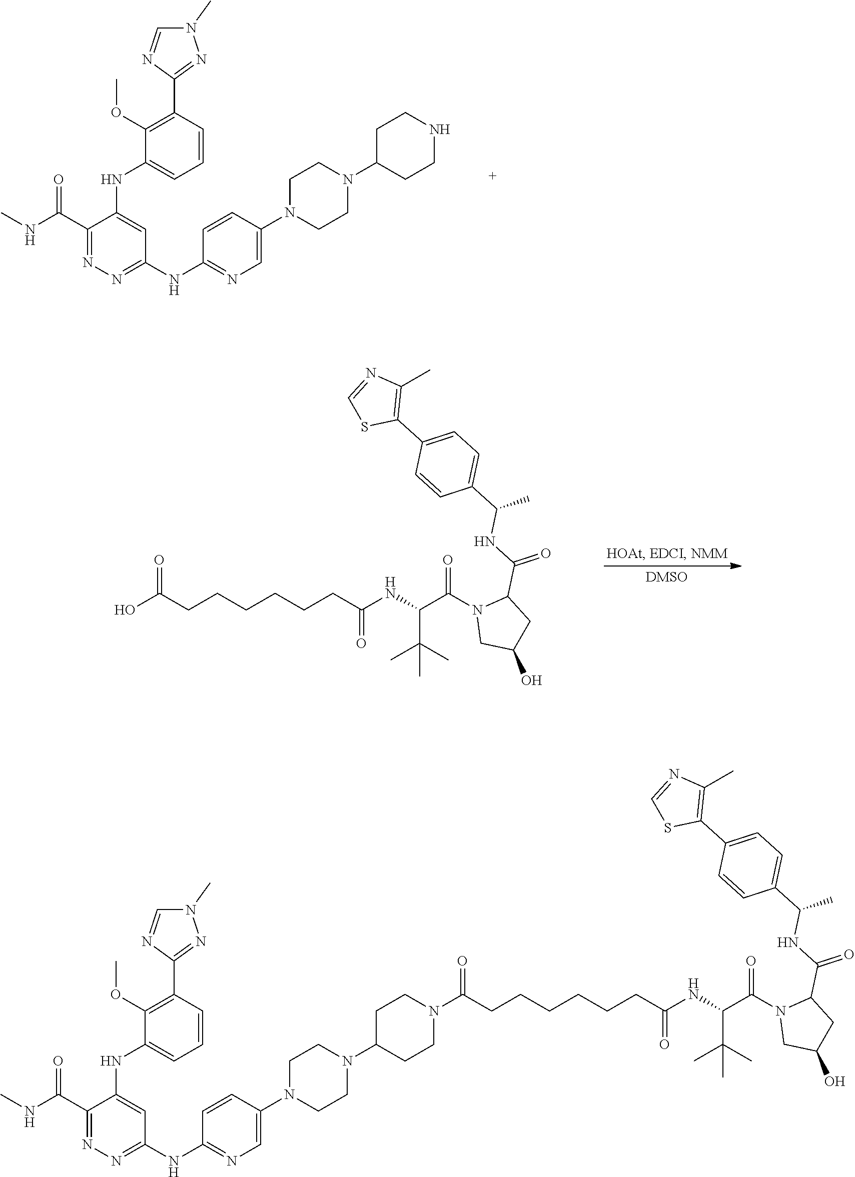 [SMILES: CNC(=O)c1nnc(Nc2ccc(N3CCN(C4CCNCC4)CC3)cn2)cc1Nc1cccc(-c2ncn(C)n2)c1OC ; Cc1ncsc1-c1ccc([C@H](C)NC(=O)C2C[C@@H](O)CN2C(=O)[C@@H](NC(=O)CCCCCCC(=O)O)C(C)(C)C)cc1 ; CNC(=O)c1nnc(Nc2ccc(N3CCN(C4CCN(C(=O)CCCCCCC(=O)N[C@H](C(=O)N5C[C@H](O)CC5C(=O)N[C@@H](C)c5ccc(-c6scnc6C)cc5)C(C)(C)C)CC4)CC3)cn2)cc1Nc1cccc(-c2ncn(C)n2)c1OC]

CPD-149 was synthesized following the same procedure for preparing CPD-145 (2.4 mg, yield: 24.3%). MS (ESI) m/z=1182.1 [M+H]⁺.

### Example 212: 6-((5-(4-(1-(9-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononanoyl)piperidin-4-yl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-150)

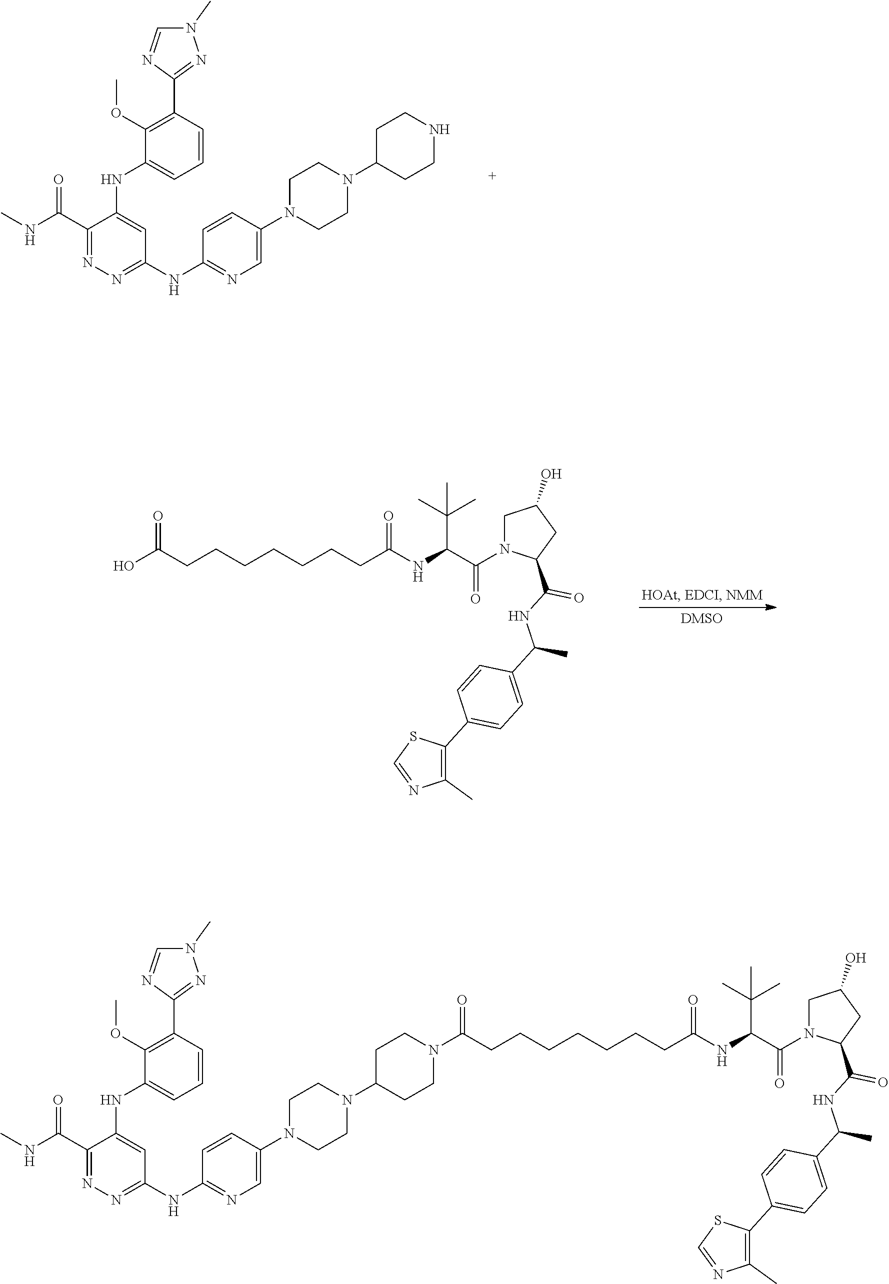 [SMILES: CNC(=O)c1nnc(Nc2ccc(N3CCN(C4CCNCC4)CC3)cn2)cc1Nc1cccc(-c2ncn(C)n2)c1OC ; Cc1ncsc1-c1ccc([C@H](C)NC(=O)[C@@H]2C[C@@H](O)CN2C(=O)[C@@H](NC(=O)CCCCCCCC(=O)O)C(C)(C)C)cc1 ; CNC(=O)c1nnc(Nc2ccc(N3CCN(C4CCN(C(=O)CCCCCCCC(=O)N[C@H](C(=O)N5C[C@H](O)C[C@H]5C(=O)N[C@@H](C)c5ccc(-c6scnc6C)cc5)C(C)(C)C)CC4)CC3)cn2)cc1Nc1cccc(-c2ncn(C)n2)c1OC]

CPD-150 was synthesized following the same procedure for preparing CPD-145 (5.3 mg, yield: 44.1%). MS (ESI) m/z=1196.0 [M+H]⁺.

### Example 213: 6-((5-(4-(1-(10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoyl)piperidin-4-yl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-151)

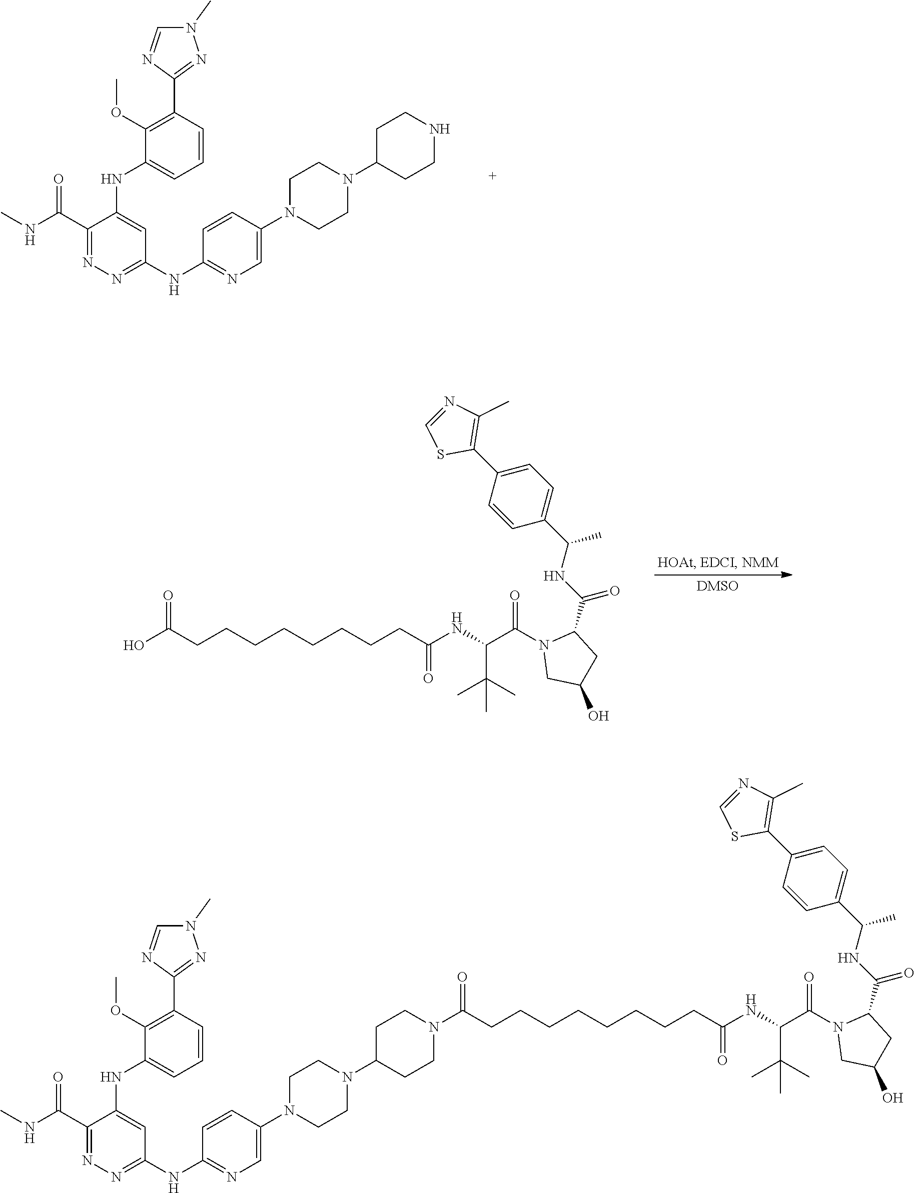 [SMILES: CNC(=O)c1nnc(Nc2ccc(N3CCN(C4CCNCC4)CC3)cn2)cc1Nc1cccc(-c2ncn(C)n2)c1OC ; Cc1ncsc1-c1ccc([C@H](C)NC(=O)[C@@H]2C[C@@H](O)CN2C(=O)[C@@H](NC(=O)CCCCCCCCC(=O)O)C(C)(C)C)cc1 ; CNC(=O)c1nnc(Nc2ccc(N3CCN(C4CCN(C(=O)CCCCCCCCC(=O)N[C@H](C(=O)N5C[C@H](O)C[C@H]5C(=O)N[C@@H](C)c5ccc(-c6scnc6C)cc5)C(C)(C)C)CC4)CC3)cn2)cc1Nc1cccc(-c2ncn(C)n2)c1OC]

CPD-151 was synthesized following the same procedure for preparing CPD-145 (6.1 mg, yield: 60.1%). MS (ESI) m/z=1210.2 [M+H]⁺.

### Example 214: 6-((5-((2-((2-(2,6-Dioxopiperidin-3-yl)-1,3-dioxoisoindolin-4-yl)amino)ethyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-152)

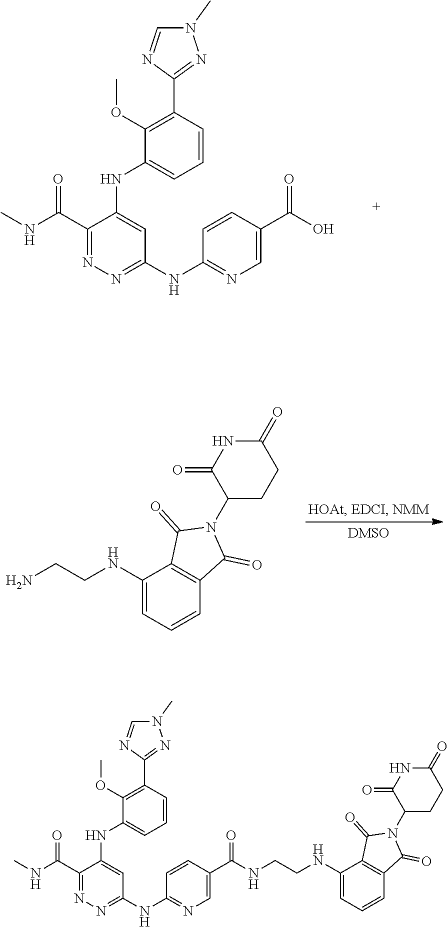 [SMILES: CNC(=O)c1nnc(Nc2ccc(C(=O)O)cn2)cc1Nc1cccc(-c2ncn(C)n2)c1OC ; NCCNc1cccc2c1C(=O)N(C1CCC(=O)NC1=O)C2=O ; CNC(=O)c1nnc(Nc2ccc(C(=O)NCCNc3cccc4c3C(=O)N(C3CCC(=O)NC3=O)C4=O)cn2)cc1Nc1cccc(-c2ncn(C)n2)c1OC]

CPD-152 was synthesized following the same procedure for preparing CPD-137 (4.1 mg, yield: 53.2%). MS (ESI) m/z=774.6 [M+H]⁺.

### Example 215: 6-((5-((3-((2-(2,6-Dioxopiperidin-3-yl)-1,3-dioxoisoindolin-4-yl)amino)propyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-153)

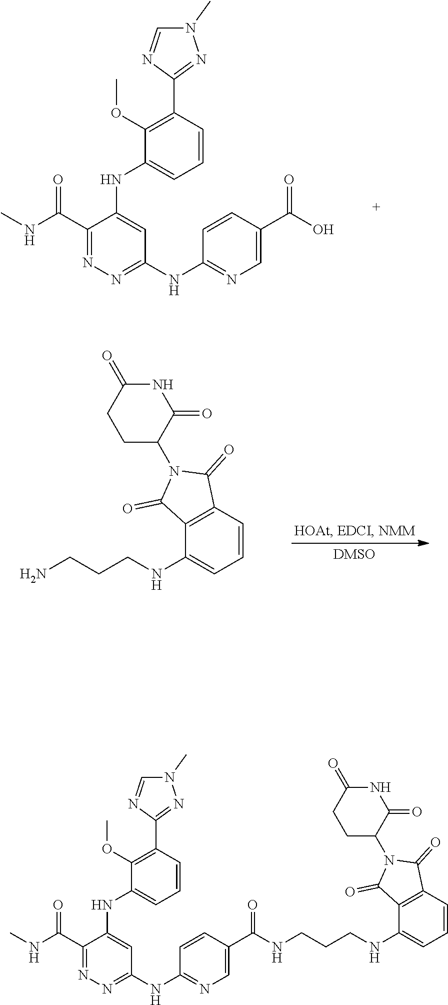 [SMILES: CNC(=O)c1nnc(Nc2ccc(C(=O)O)cn2)cc1Nc1cccc(-c2ncn(C)n2)c1OC ; NCCCNc1cccc2c1C(=O)N(C1CCC(=O)NC1=O)C2=O ; CNC(=O)c1nnc(Nc2ccc(C(=O)NCCCNc3cccc4c3C(=O)N(C3CCC(=O)NC3=O)C4=O)cn2)cc1Nc1cccc(-c2ncn(C)n2)c1OC]

CPD-153 was synthesized following the same procedure for preparing CPD-137 (4.2 mg, yield: 53.2%). MS (ESI) m/z=788.7 [M+H]⁺.

### Example 216: 6-((5-((4-((2-(2,6-Dioxopiperidin-3-yl)-1,3-dioxoisoindolin-4-yl)amino)butyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-154)

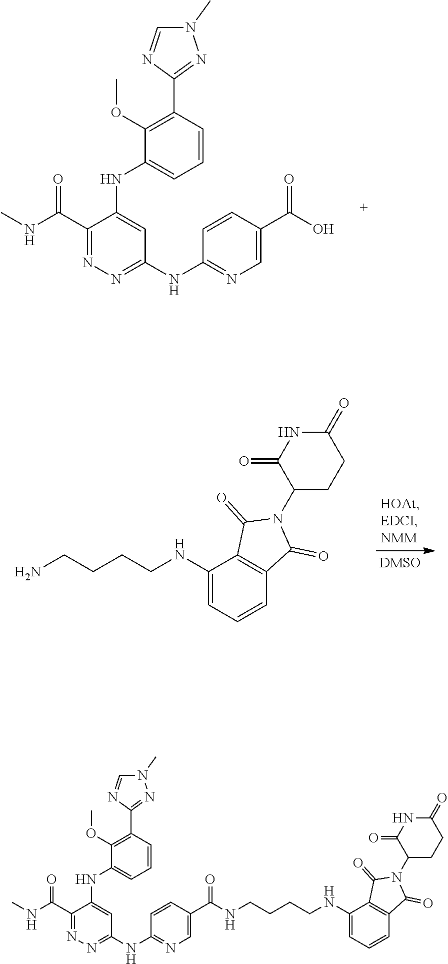 [SMILES: CNC(=O)c1nnc(Nc2ccc(C(=O)O)cn2)cc1Nc1cccc(-c2ncn(C)n2)c1OC ; NCCCCNc1cccc2c1C(=O)N(C1CCC(=O)NC1=O)C2=O ; CNC(=O)c1nnc(Nc2ccc(C(=O)NCCCCNc3cccc4c3C(=O)N(C3CCC(=O)NC3=O)C4=O)cn2)cc1Nc1cccc(-c2ncn(C)n2)c1OC]

CPD-154 was synthesized following the same procedure for preparing CPD-137 (4.6 mg, yield: 57.5%). MS (ESI) m/z=802.8 [M+H]⁺.

### Example 217: 6-((5-((5-((2-(2,6-Dioxopiperidin-3-yl)-1,3-dioxoisoindolin-4-yl)amino)pentyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-155)

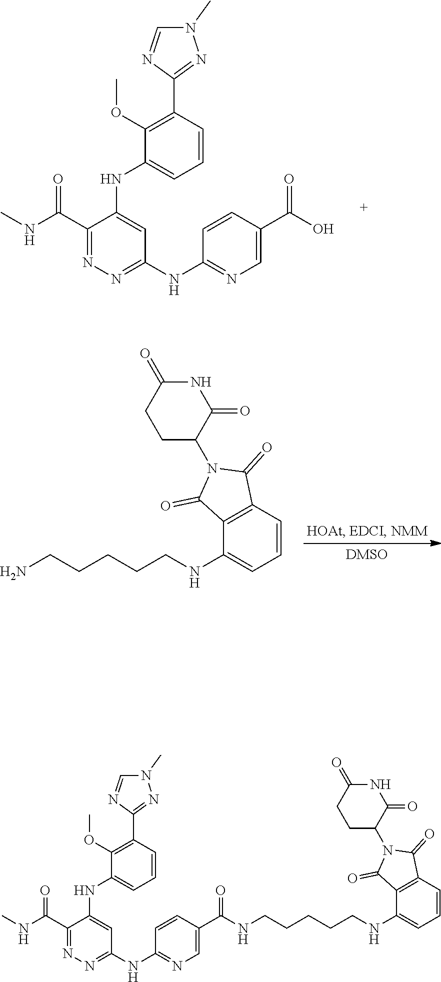 [SMILES: CNC(=O)c1nnc(Nc2ccc(C(=O)O)cn2)cc1Nc1cccc(-c2ncn(C)n2)c1OC ; NCCCCCNc1cccc2c1C(=O)N(C1CCC(=O)NC1=O)C2=O ; CNC(=O)c1nnc(Nc2ccc(C(=O)NCCCCCNc3cccc4c3C(=O)N(C3CCC(=O)NC3=O)C4=O)cn2)cc1Nc1cccc(-c2ncn(C)n2)c1OC]

CPD-155 was synthesized following the same procedure for preparing CPD-137 (4.6 mg, yield: 56.7%). MS (ESI) m/z=816.8 [M+H]⁺.

### Example 218: 6-((5-((6-((2-(2,6-Dioxopiperidin-3-yl)-1,3-dioxoisoindolin-4-yl)amino)hexyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-156)

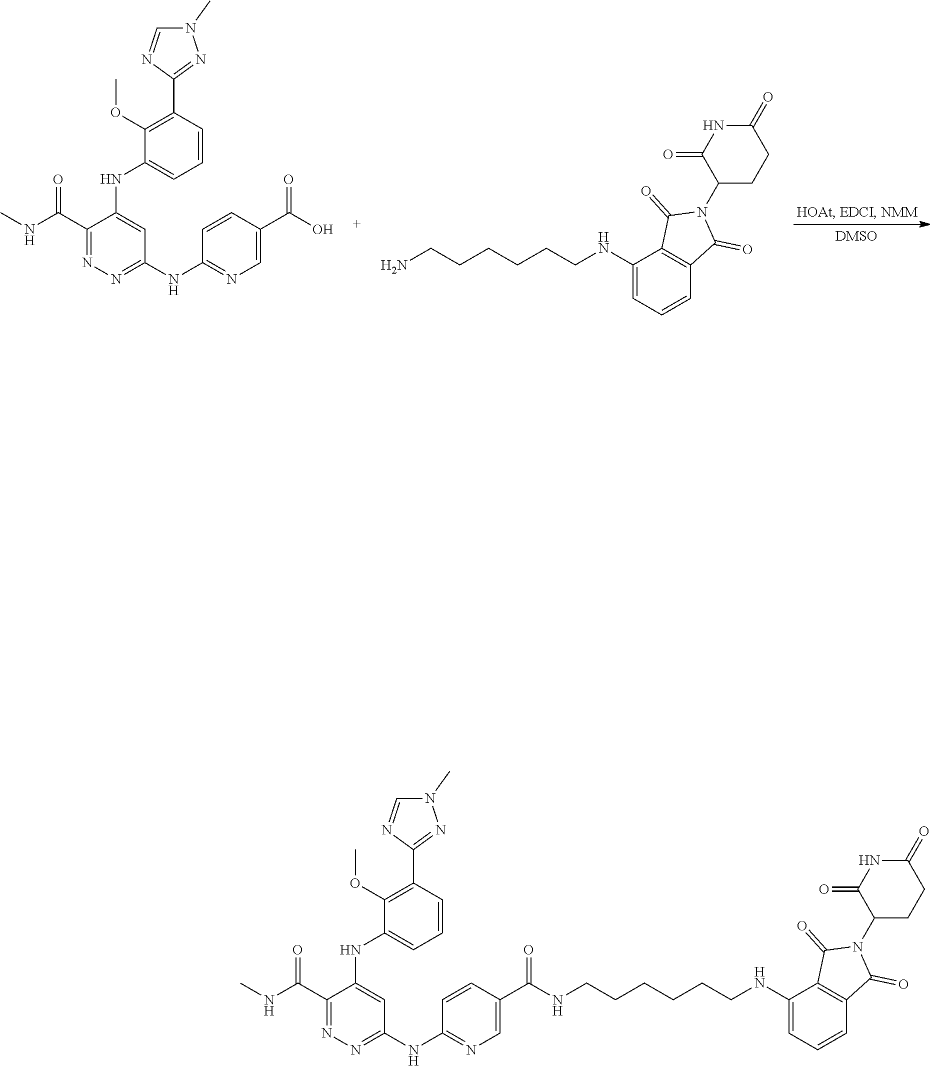 [SMILES: CNC(=O)c1nnc(Nc2ccc(C(=O)O)cn2)cc1Nc1cccc(-c2ncn(C)n2)c1OC ; NCCCCCCNc1cccc2c1C(=O)N(C1CCC(=O)NC1=O)C2=O ; CNC(=O)c1nnc(Nc2ccc(C(=O)NCCCCCCNc3cccc4c3C(=O)N(C3CCC(=O)NC3=O)C4=O)cn2)cc1Nc1cccc(-c2ncn(C)n2)c1OC]

CPD-156 was synthesized following the same procedure for preparing CPD-137 (4.9 mg, yield: 59.1%). MS (ESI) m/z=830.8 [M+H]⁺.

### Example 219: 6-((5-((7-((2-(2,6-Dioxopiperidin-3-yl)-1,3-dioxoisoindolin-4-yl)amino)heptyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-157)

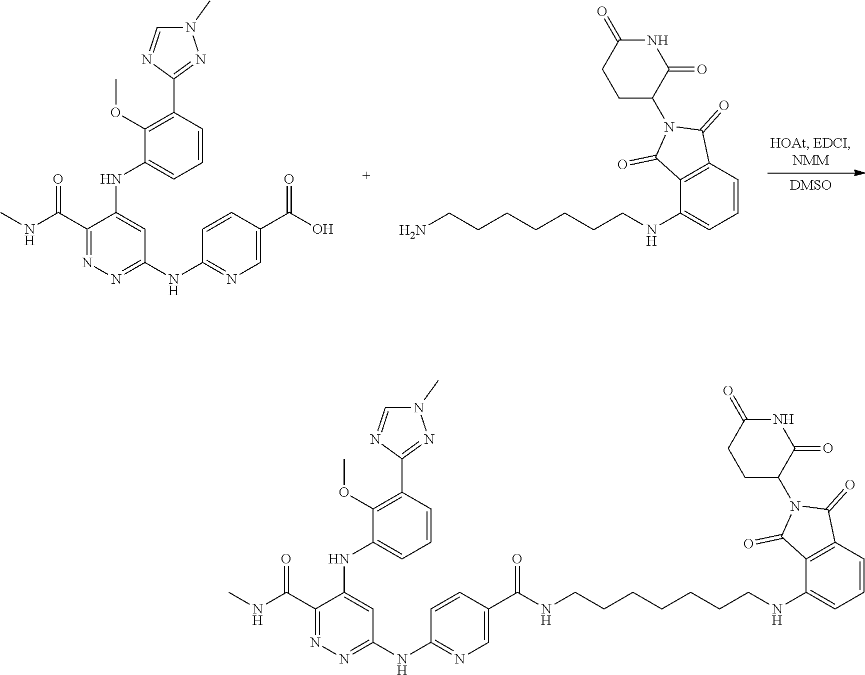 [SMILES: CNC(=O)c1nnc(Nc2ccc(C(=O)O)cn2)cc1Nc1cccc(-c2ncn(C)n2)c1OC ; NCCCCCCCNc1cccc2c1C(=O)N(C1CCC(=O)NC1=O)C2=O ; CNC(=O)c1nnc(Nc2ccc(C(=O)NCCCCCCCNc3cccc4c3C(=O)N(C3CCC(=O)NC3=O)C4=O)cn2)cc1Nc1cccc(-c2ncn(C)n2)c1OC]

CPD-157 was synthesized following the same procedure for preparing CPD-137 (5.4 mg, yield: 64.2%). MS (ESI) m/z=844.8 [M+H]⁺.

### Example 220: 6-((5-((8-((2-(2,6-Dioxopiperidin-3-yl)-1,3-dioxoisoindolin-4-yl)amino)octyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-158)

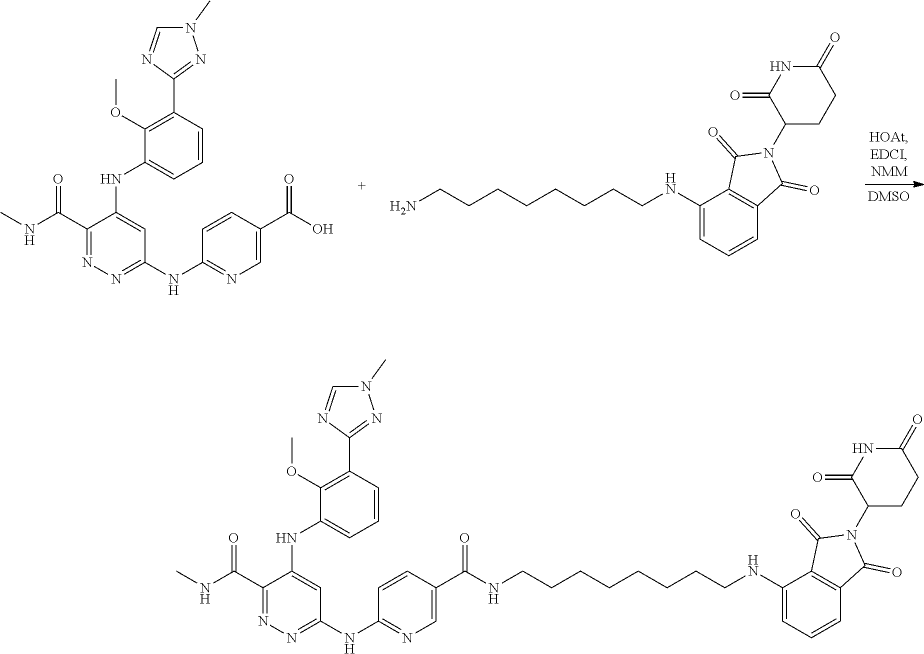 [SMILES: CNC(=O)c1nnc(Nc2ccc(C(=O)O)cn2)cc1Nc1cccc(-c2ncn(C)n2)c1OC ; NCCCCCCCCNc1cccc2c1C(=O)N(C1CCC(=O)NC1=O)C2=O ; CNC(=O)c1nnc(Nc2ccc(C(=O)NCCCCCCCCNc3cccc4c3C(=O)N(C3CCC(=O)NC3=O)C4=O)cn2)cc1Nc1cccc(-c2ncn(C)n2)c1OC]

CPD-158 was synthesized following the same procedure for preparing CPD-137 (3.1 mg, yield: 36.4%). MS (ESI) m/z=858.8 [M+H]⁺.

### Example 221: 6-((5-((2-(2-((2-(2,6-Dioxopiperidin-3-yl)-1,3-dioxoisoindolin-4-yl)amino)ethoxy)ethyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-159)

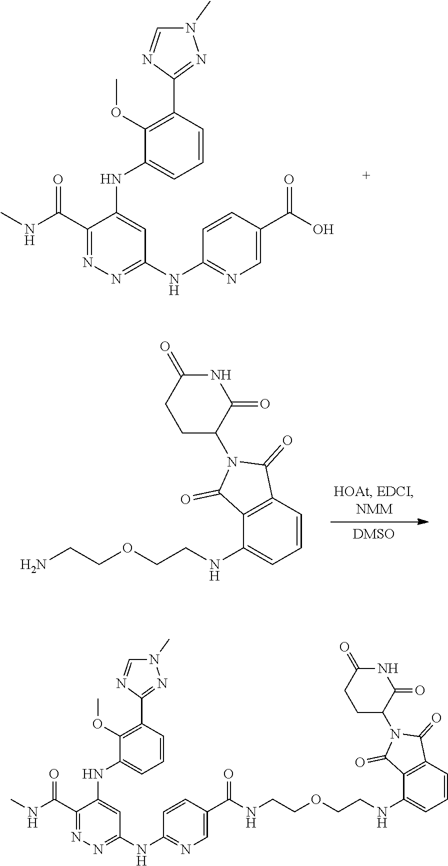 [SMILES: CNC(=O)c1nnc(Nc2ccc(C(=O)O)cn2)cc1Nc1cccc(-c2ncn(C)n2)c1OC ; NCCOCCNc1cccc2c1C(=O)N(C1CCC(=O)NC1=O)C2=O ; CNC(=O)c1nnc(Nc2ccc(C(=O)NCCOCCNc3cccc4c3C(=O)N(C3CCC(=O)NC3=O)C4=O)cn2)cc1Nc1cccc(-c2ncn(C)n2)c1OC]

CPD-159 was synthesized following the same procedure for preparing CPD-137 (4.1 mg, yield: 50.6%). MS (ESI) m/z=818.7 [M+H]⁺.

### Example 222: 6-((5-((1-(5-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentyl)piperidin-4-yl)ethynyl)pyridin-2-yl)amino)-4-((2-methoxy-3-(1-methyl-1H-1,2,4-triazol-3-yl)phenyl)amino)-N-methylpyridazine-3-carboxamide (CPD-160)

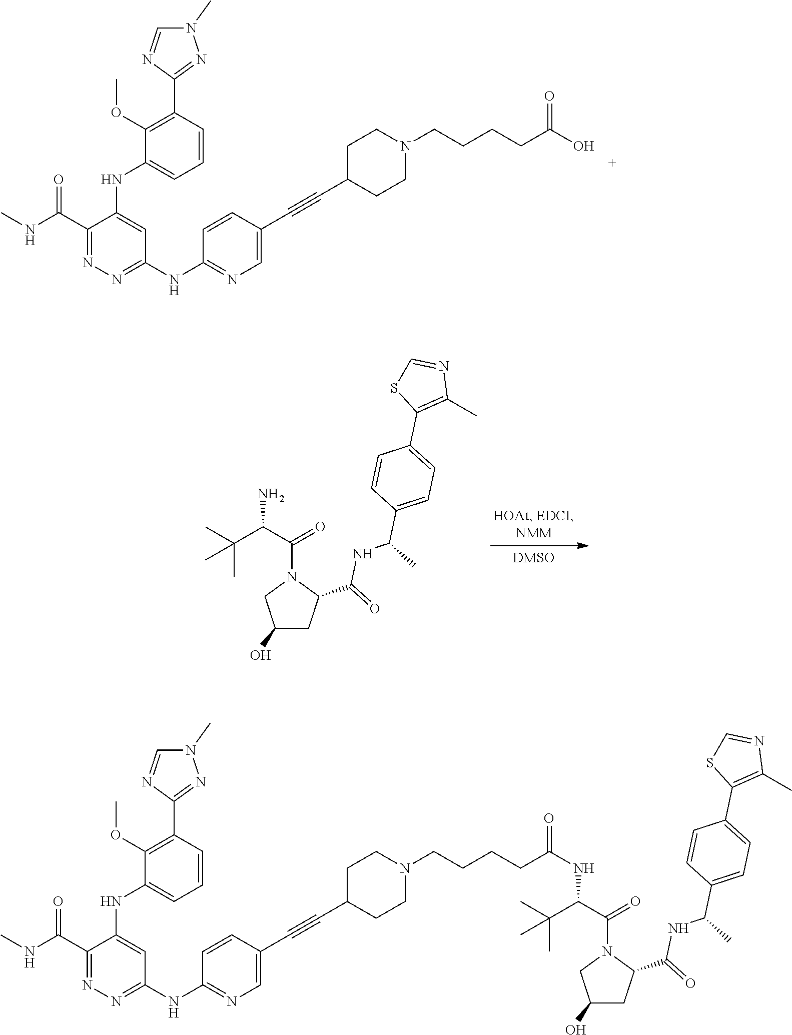

CPD-160 was synthesized following the same procedure for preparing CPD-113 (19.2 mg, yield: 62.3%). MS (ESI) m/z=1065.9 [M+H]⁺.

### Example 223: 6-((5-((3-(2-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclobutyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-161)

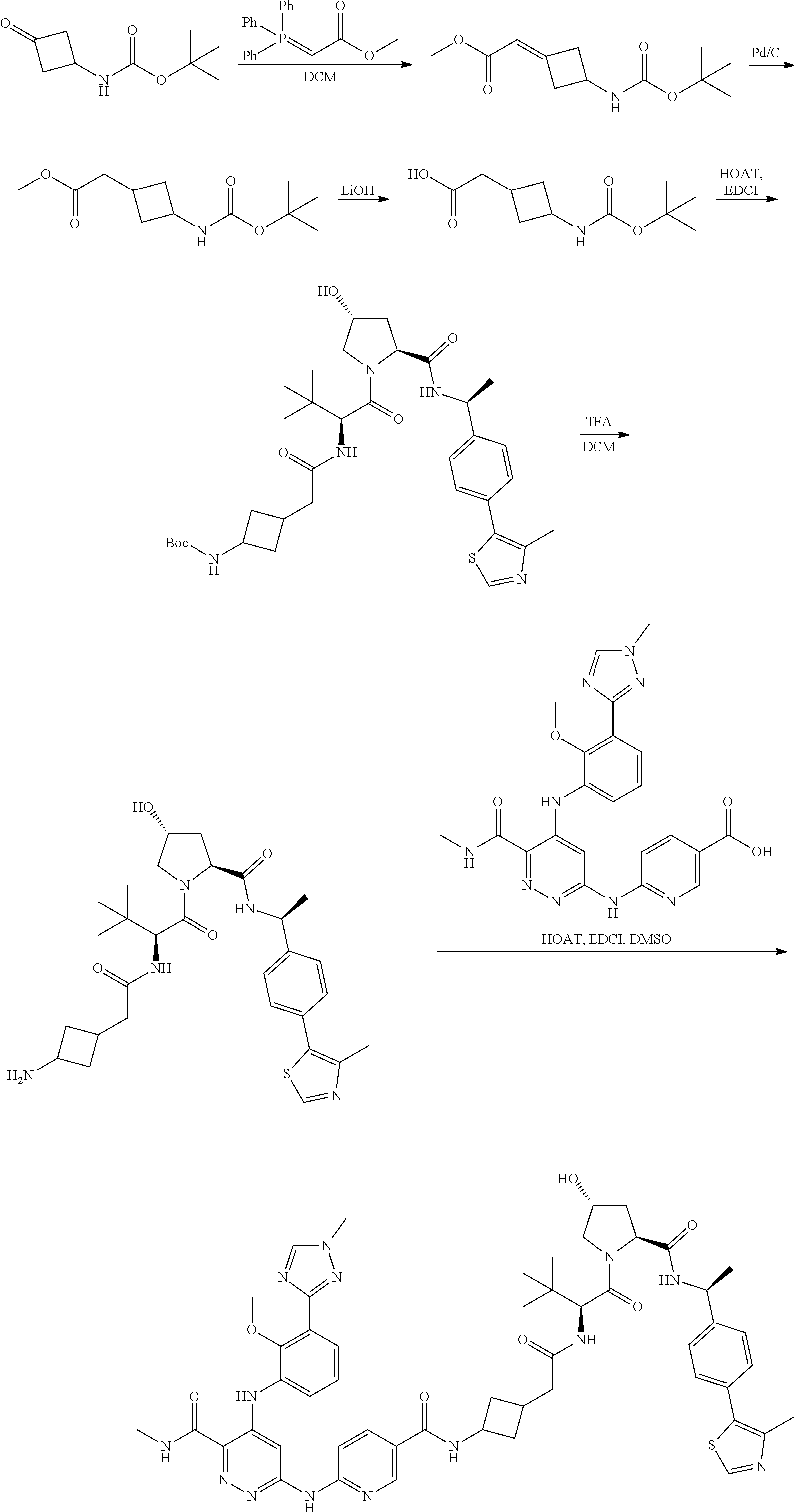

### Step 1. Synthesis of methyl 2-(3-((tert-butoxycarbonyl)amino)cyclobutylidene)acetate

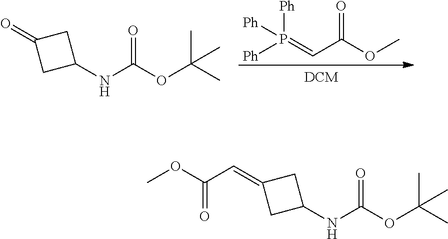

To a solution of tert-butyl N-(3-oxocyclobutyl)carbamate (1.85 g, 9.99 mmol) in DCM (40 mL) was added methyl 2-(triphenyl-phosphanylidene)acetate (3.34 g, 9.99 mmol). After the mixture was stirred at rt for 48 h, it was concentrated and purified by silica gel chromatography (petroleum ether/EtOAc=10/1) to give the title compound (1.9 g, 78.8% yield) as a white solid.

### Step 2. Synthesis of methyl 2-(3-((tert-butoxycarbonyl)amino)cyclobutyl)acetate

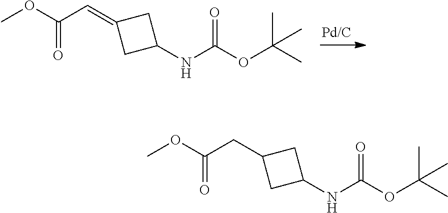

To a solution of methyl 2-[3-(tert-butoxycarbonylamino)cyclobutylidene]acetate (1.9 g, 7.87 mmol) in MeOH (50 mL) was added Pd/C (200 mg). After the reaction mixture was stirred at rt under H₂ for 16 h, it was filtered through Celite. The filtrate was concentrated under reduced pressure to give the title compound (1.9 g, 99.2% yield) as a white solid.

### Step 3. Synthesis of 2-(3-((tert-butoxycarbonyl)amino)cyclobutyl)acetic acid

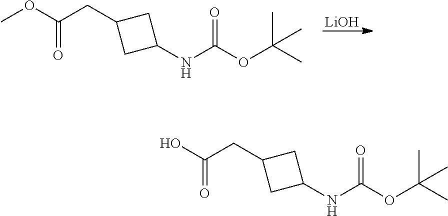

To a solution of methyl 2-[3-(tert-butoxycarbonylamino)cyclobutyl]acetate (1.9 g, 7.81 mmol) in MeOH (20 mL) and water (20 mL) was added LiOH·H₂O (1.64 g, 39.05 mmol). After the reaction mixture was stirred at rt for 2 h, it was concentrated to remove MeOH. After the pH of the reaction was adjusted to 3 with HCl (1N), it was extracted with DCM (30 mL×3). The combined organic layers were dried over Na₂SO₄, filtered, and concentrated to give the title compound (1.7 g, 95% yield) as a white solid.

### Step 4. Synthesis of tert-butyl (3-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclobutyl)carbamate

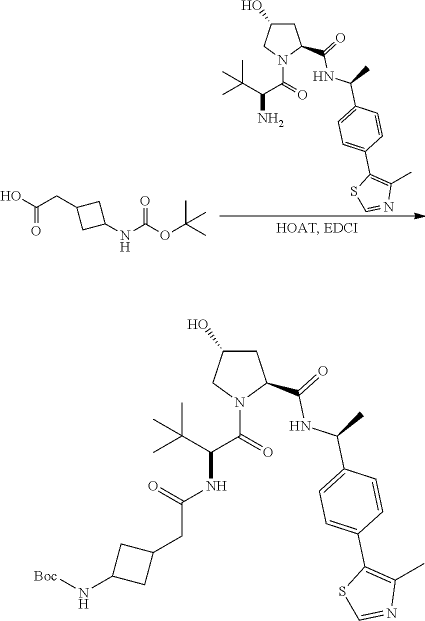

To a solution of (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (81.5 mg, 183.6 μmol) in DMSO (10 mL) were added 2-(3-((tert-butoxycarbonyl)amino)cyclobutyl)acetic acid (42.09 mg, 183.59 μmol), HOAT (37.45 mg, 275.38 μmol), EDCI (52.79 mg, 275.38 μmol) and TEA (55.73 mg, 550.76 μmol). After the reaction mixture was stirred at rt for 16 h, it was purified by prep-HPLC to give the title compound (125 mg, 88.4% yield) as a white solid. MS (ESI) m/z=656.7 [M+H]⁺.

### Step 5. Synthesis of 6-((5-((3-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclobutyl)carbamoyl)pyridin-2-yl)amino)-4-((2-methoxy-3-(1-methyl-1H-1,2,4-triazol-3-yl)phenyl)amino)-N-methylpyridazine-3-carboxamide

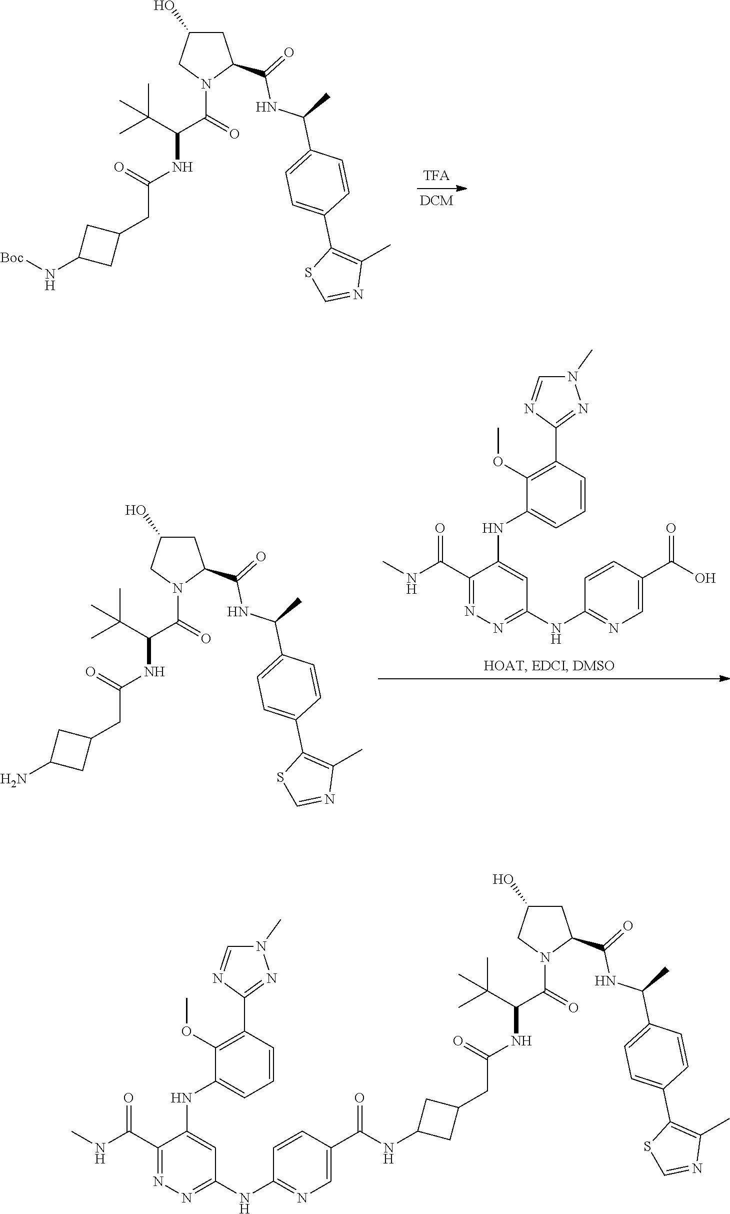

CPD-161 was synthesized following the same procedure for preparing CPD-137 (23 mg, yield: 67.4%). MS (ESI) m/z=1014.0 [M+H]⁺.

### Example 224: 6-((5-(((8-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctyl)amino)methyl)pyridin-2-yl)amino)-4-((2-methoxy-3-(1-methyl-1H-1,2,4-triazol-3-yl)phenyl)amino)-N-methylpyridazine-3-carboxamide (CPD-162)

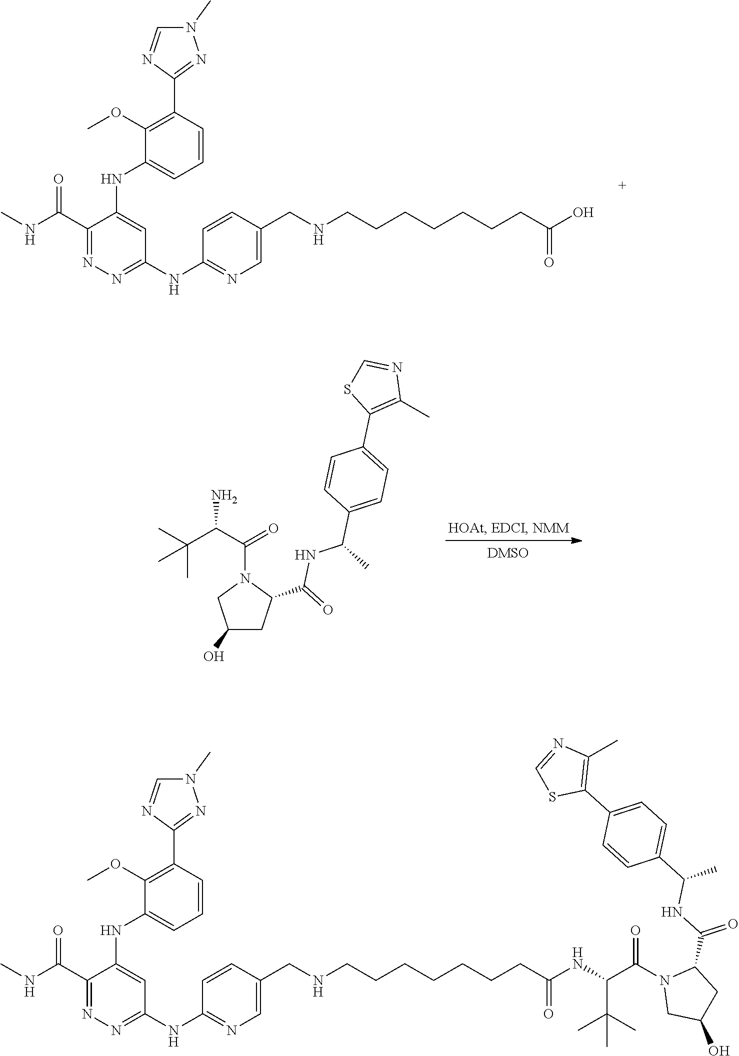 [SMILES: CNC(=O)c1nnc(Nc2ccc(CNCCCCCCCC(=O)O)cn2)cc1Nc1cccc(-c2ncn(C)n2)c1OC ; Cc1ncsc1-c1ccc([C@H](C)NC(=O)[C@@H]2C[C@@H](O)CN2C(=O)[C@@H](N)C(C)(C)C)cc1 ; CNC(=O)c1nnc(Nc2ccc(CNCCCCCCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC]

CPD-162 was synthesized following the same procedure for preparing CPD-113 (2.2 mg, yield: 17.8%). MS (ESI) m/z=1030.0 [M+H]⁺.

### Example 225: 6-((5-((4-((2-(2,6-Dioxopiperidin-3-yl)-1,3-dioxoisoindolin-5-yl)amino)butyl)carbamoyl)pyridin-2-yl)amino)-4-((2-methoxy-3-(1-methyl-1H-1,2,4-triazol-3-yl)phenyl)amino-N-methylpyridazine-3-carboxamide (CPD-163)

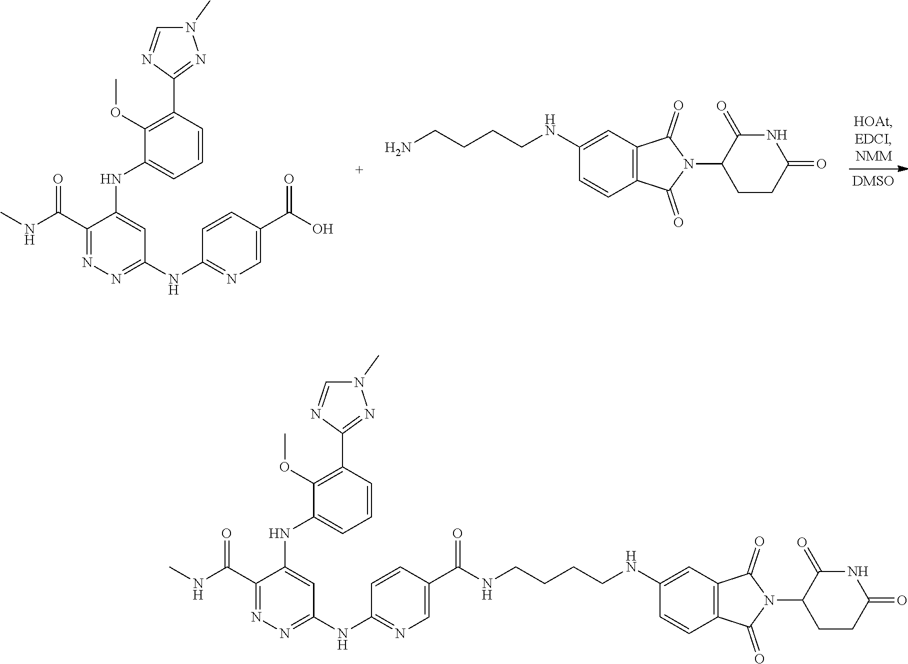 [SMILES: CNC(=O)c1nnc(Nc2ccc(C(=O)O)cn2)cc1Nc1cccc(-c2ncn(C)n2)c1OC ; NCCCCNc1ccc2c(c1)C(=O)N(C1CCC(=O)NC1=O)C2=O ; CNC(=O)c1nnc(Nc2ccc(C(=O)NCCCCNc3ccc4c(c3)C(=O)N(C3CCC(=O)NC3=O)C4=O)cn2)cc1Nc1cccc(-c2ncn(C)n2)c1OC]

CPD-163 was synthesized following the same procedure for preparing CPD-137 (4.2 mg, yield: 52.5%). MS (ESI) m/z=802.8 [M+H]⁺.

### Example 226: 6-((5-((5-((2-(2,6-Dioxopiperidin-3-yl)-1,3-dioxoisoindolin-5-yl)amino)pentyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-164)

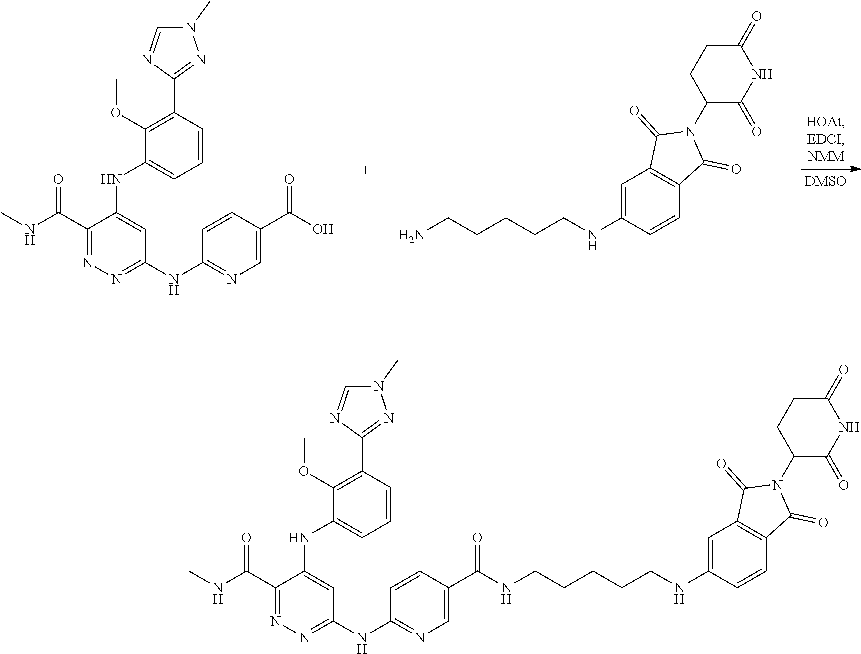 [SMILES: CNC(=O)c1nnc(Nc2ccc(C(=O)O)cn2)cc1Nc1cccc(-c2ncn(C)n2)c1OC ; NCCCCCNc1ccc2c(c1)C(=O)N(C1CCC(=O)NC1=O)C2=O ; CNC(=O)c1nnc(Nc2ccc(C(=O)NCCCCCNc3ccc4c(c3)C(=O)N(C3CCC(=O)NC3=O)C4=O)cn2)cc1Nc1cccc(-c2ncn(C)n2)c1OC]

CPD-164 was synthesized following the same procedure for preparing CPD-137 (3.7 mg, yield: 45.6%). MS (ESI) m/z=816.8 [M+H]⁺.

### Example 227: 6-((5-((6-((2-(2,6-Dioxopiperidin-3-yl)-1,3-dioxoisoindolin-5-yl)amino)hexyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-165)

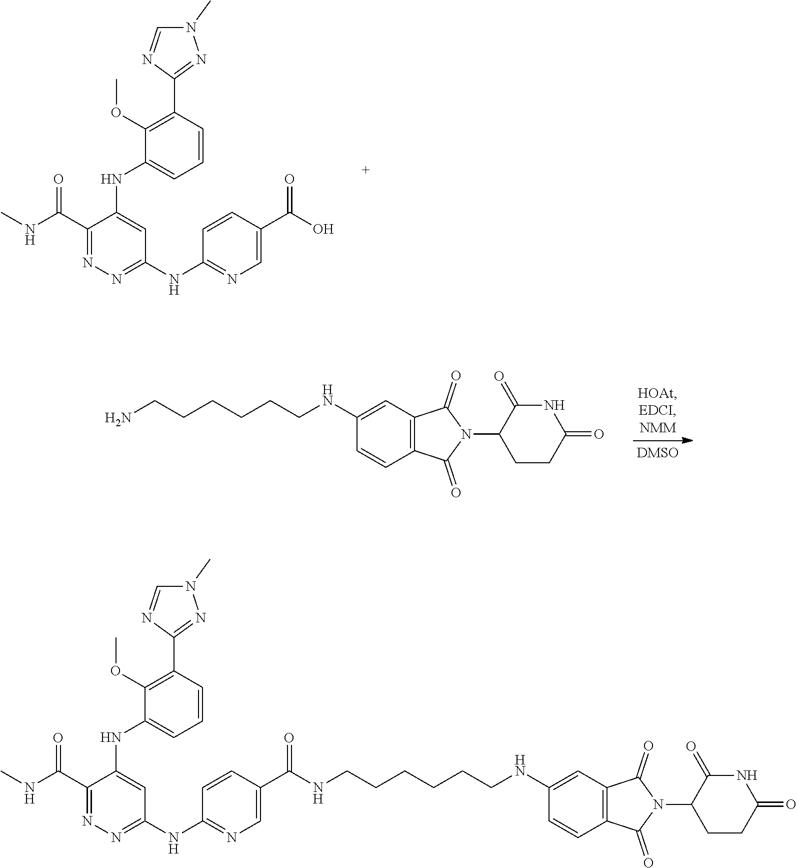 [SMILES: CNC(=O)c1nnc(Nc2ccc(C(=O)O)cn2)cc1Nc1cccc(-c2ncn(C)n2)c1OC ; NCCCCCCNc1ccc2c(c1)C(=O)N(C1CCC(=O)NC1=O)C2=O ; CNC(=O)c1nnc(Nc2ccc(C(=O)NCCCCCCNc3ccc4c(c3)C(=O)N(C3CCC(=O)NC3=O)C4=O)cn2)cc1Nc1cccc(-c2ncn(C)n2)c1OC]

CPD-165 was synthesized following the same procedure for preparing CPD-137 (3.5 mg, yield: 42.1%). MS (ESI) m/z=830.9 [M+H]⁺.

### Example 228: 6-((5-((7-((2-(2,6-Dioxopiperidin-3-yl)-1,3-dioxoisoindolin-5-yl)amino)heptyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-166)

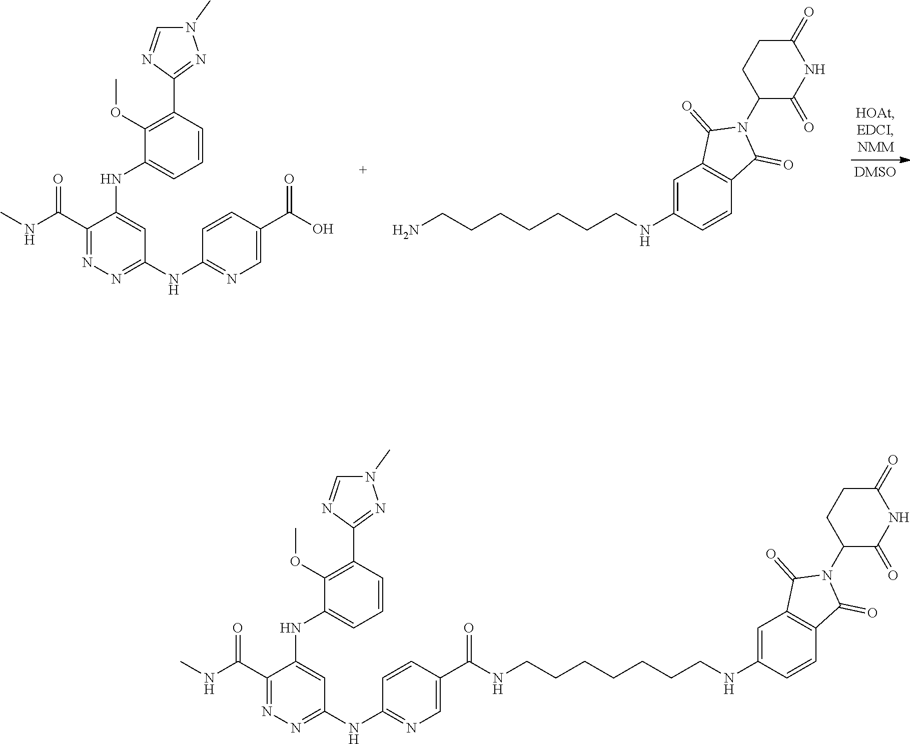 [SMILES: CNC(=O)c1nnc(Nc2ccc(C(=O)O)cn2)cc1Nc1cccc(-c2ncn(C)n2)c1OC ; NCCCCCCCNc1ccc2c(c1)C(=O)N(C1CCC(=O)NC1=O)C2=O ; CNC(=O)c1nnc(Nc2ccc(C(=O)NCCCCCCCNc3ccc4c(c3)C(=O)N(C3CCC(=O)NC3=O)C4=O)cn2)cc1Nc1cccc(-c2ncn(C)n2)c1OC]

CPD-166 was synthesized following the same procedure for preparing CPD-137 (3.4 mg, yield: 40.4%). MS (ESI) m/z=844.9 [M+H]⁺.

### Example 229: 6-((5-((8-((2-(2,6-Dioxopiperidin-3-yl)-1,3-dioxoisoindolin-5-yl)amino)octyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-167)

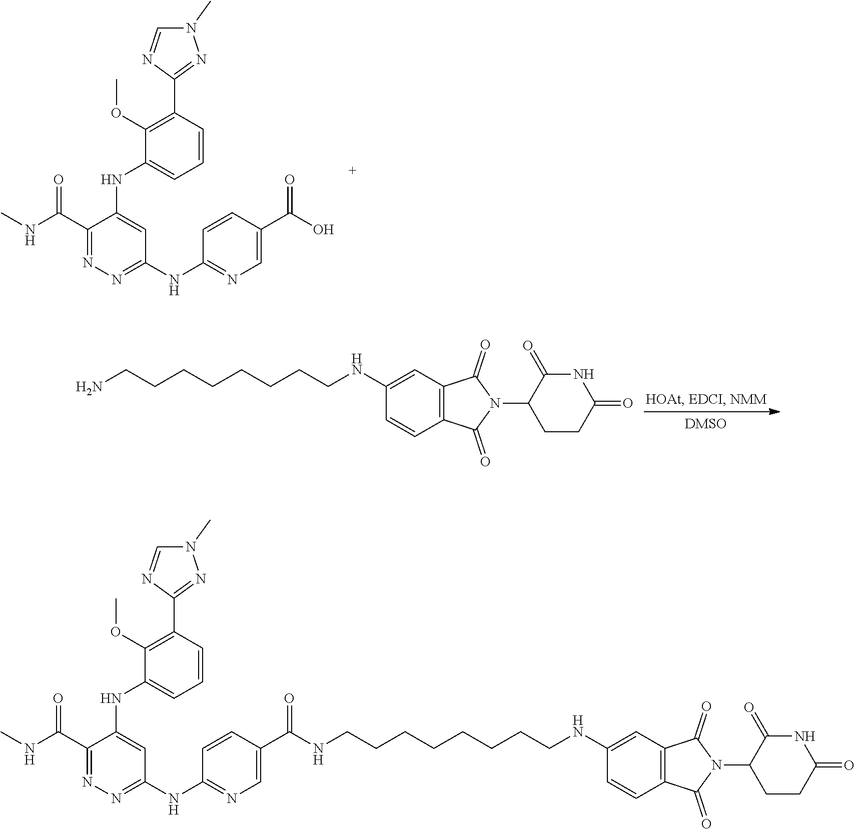 [SMILES: CNC(=O)c1nnc(Nc2ccc(C(=O)O)cn2)cc1Nc1cccc(-c2ncn(C)n2)c1OC ; NCCCCCCCCNc1ccc2c(c1)C(=O)N(C1CCC(=O)NC1=O)C2=O ; CNC(=O)c1nnc(Nc2ccc(C(=O)NCCCCCCCCNc3ccc4c(c3)C(=O)N(C3CCC(=O)NC3=O)C4=O)cn2)cc1Nc1cccc(-c2ncn(C)n2)c1OC]

CPD-167 was synthesized following the same procedure for preparing CPD-137 (4.3 mg, yield: 50.5%). MS (ESI) m/z=858.9 [M+H]⁺.

### Example 230: 6-((5-((2-(2-((2-(2,6-Dioxopiperidin-3-yl)-1,3-dioxoisoindolin-5-yl)amino)ethoxy)ethyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-168)

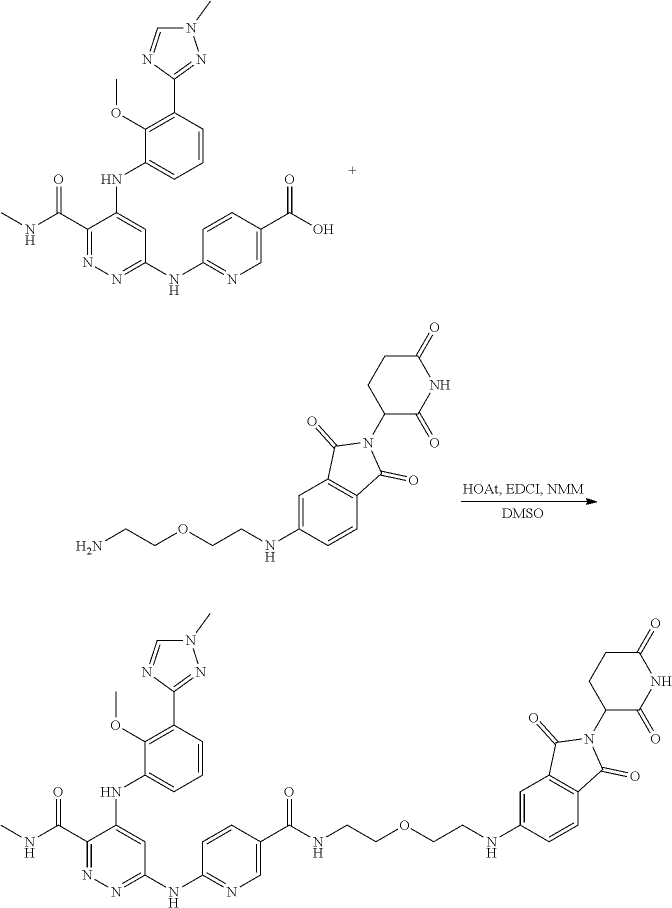 [SMILES: CNC(=O)c1nnc(Nc2ccc(C(=O)O)cn2)cc1Nc1cccc(-c2ncn(C)n2)c1OC ; NCCOCCNc1ccc2c(c1)C(=O)N(C1CCC(=O)NC1=O)C2=O ; CNC(=O)c1nnc(Nc2ccc(C(=O)NCCOCCNc3ccc4c(c3)C(=O)N(C3CCC(=O)NC3=O)C4=O)cn2)cc1Nc1cccc(-c2ncn(C)n2)c1OC]

CPD-168 was synthesized following the same procedure for preparing CPD-137 (2.8 mg, yield: 34.5%). MS (ESI) m/z=818.8 [M+H]⁺.

### Example 231: 6-((5-(4-(8-(((S)-1-((2S,4R)-4-Hydroxy-2-(((S)-1-phenylethyl)carbamoyl)pyrrolidin-1-yl)-3,3-dimethyl-1-oxobutan-2-yl)amino)-8-oxooctanoyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-169)

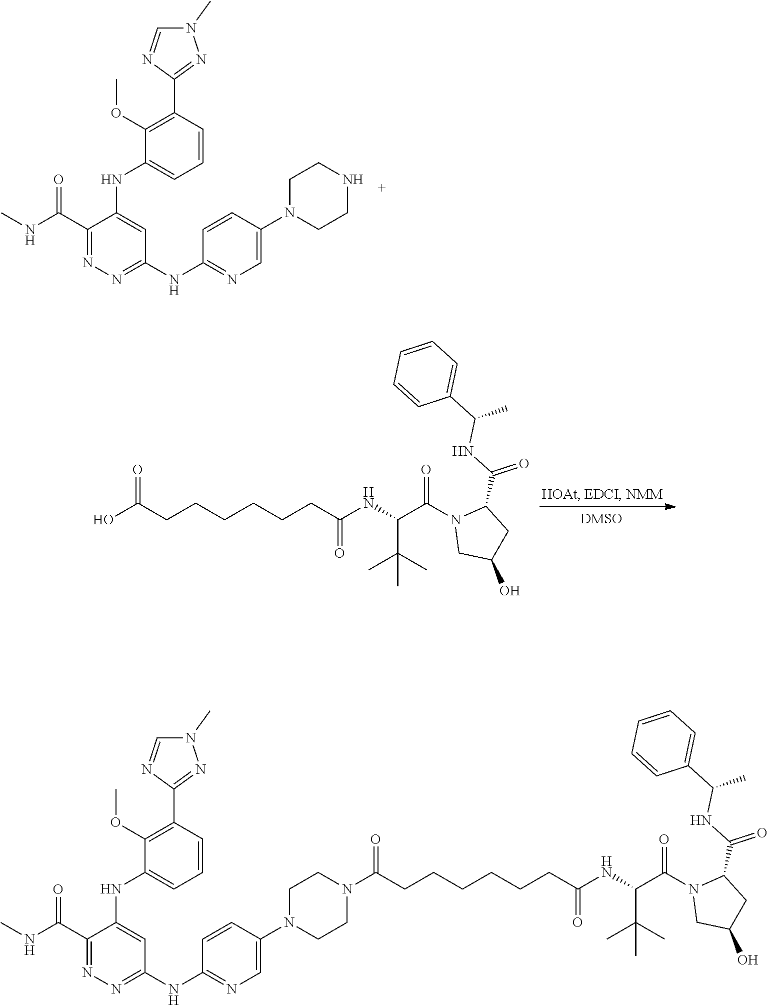 [SMILES: CNC(=O)c1nnc(Nc2ccc(N3CCNCC3)cn2)cc1Nc1cccc(-c2ncn(C)n2)c1OC ; C[C@H](NC(=O)[C@@H]1C[C@@H](O)CN1C(=O)[C@@H](NC(=O)CCCCCCC(=O)O)C(C)(C)C)c1ccccc1 ; CNC(=O)c1nnc(Nc2ccc(N3CCN(C(=O)CCCCCCC(=O)N[C@H](C(=O)N4C[C@H](O)C[C@H]4C(=O)N[C@@H](C)c4ccccc4)C(C)(C)C)CC3)cn2)cc1Nc1cccc(-c2ncn(C)n2)c1OC]

CPD-169 was synthesized following the same procedure for preparing CPD-096 (13 mg, yield: 44%). MS (ESI) m/z=1002.2 [M+H]⁺.

### Example 232: 6-((5-(4-(8-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctanoyl)piperazin-1-yl)pyridin-2-yl)amino)-4-((2-methoxyphenyl)amino)-N-methylnicotinamide (CPD-170)

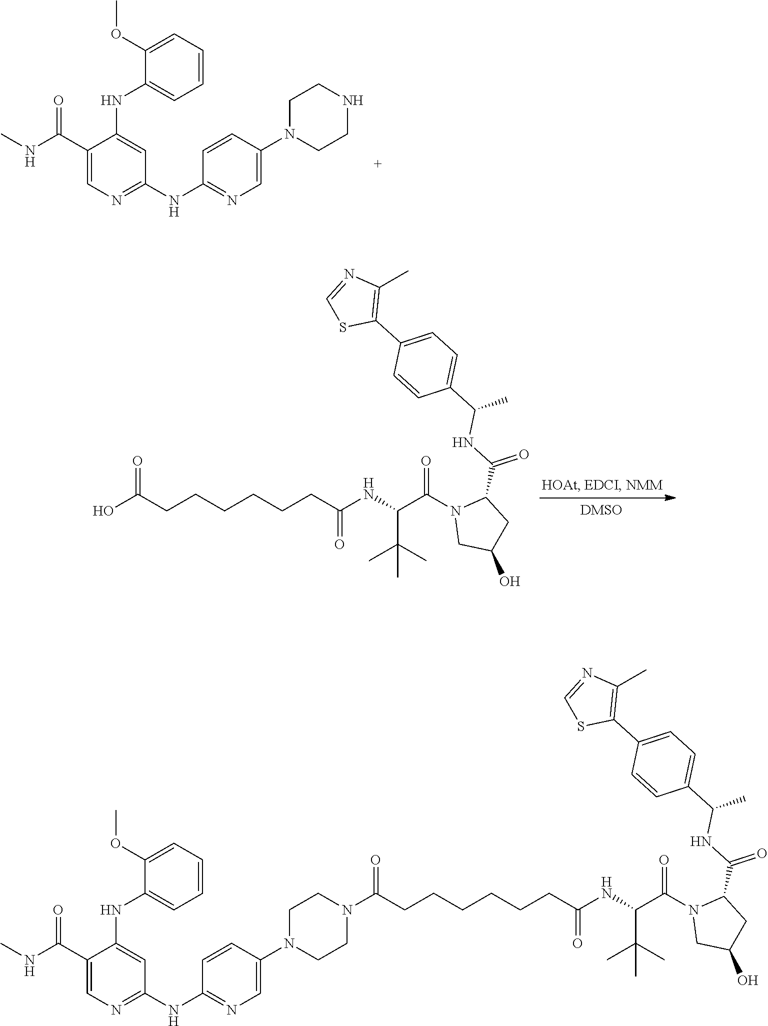 [SMILES: CNC(=O)c1cnc(Nc2ccc(N3CCNCC3)cn2)cc1Nc1ccccc1OC ; Cc1ncsc1-c1ccc([C@H](C)NC(=O)[C@@H]2C[C@@H](O)CN2C(=O)[C@@H](NC(=O)CCCCCCC(=O)O)C(C)(C)C)cc1 ; CNC(=O)c1cnc(Nc2ccc(N3CCN(C(=O)CCCCCCC(=O)N[C@H](C(=O)N4C[C@H](O)C[C@H]4C(=O)N[C@@H](C)c4ccc(-c5scnc5C)cc4)C(C)(C)C)CC3)cn2)cc1Nc1ccccc1OC]

CPD-170 was synthesized following the same procedure for preparing CPD-096 (3.8 mg, yield: 10.8%). MS (ESI) m/z=1017.1 [M+H]⁺.

### Example 233: 6-((5-(1-(8-(((S)-1-((2S,4R)-2-((4-Cyanobenzyl)carbamoyl)-4-hydroxypyrrolidin-1-yl)-3,3-dimethyl-1-oxobutan-2-yl)amino)-8-oxooctanoyl)piperidin-4-yl)pyridin-2-yl)amino)-4-((2-methoxy-3-(1-meth 1-1H-1,2,4-triazol-3-yl)phenyl)amino)-N-methylpyridazine-3-carboxamide (CPD-171

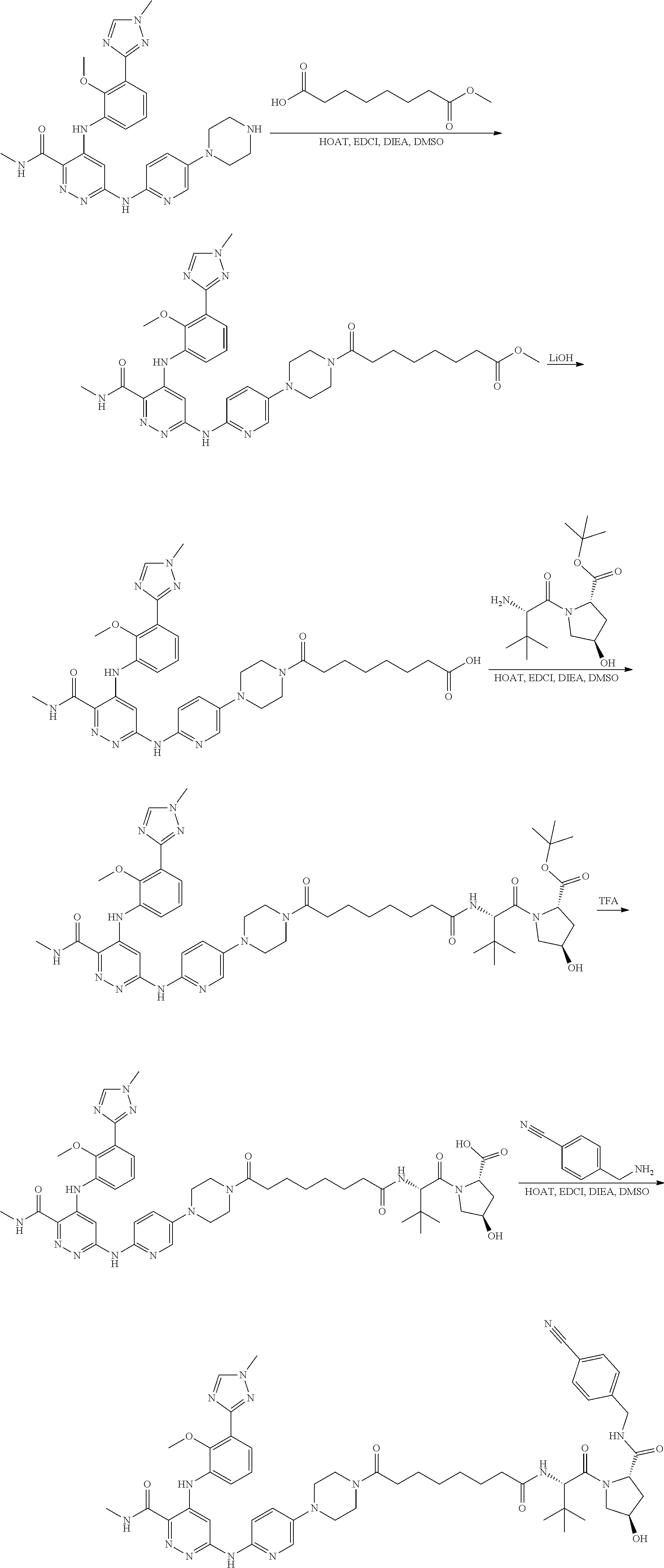

### Step 1. Synthesis of methyl 8-(4-(6-((5-((2-methoxy-3-(1-methyl-1H-1,2,4-triazol-3-yl)phenyl)amino)-6-(methylcarbamoyl)pyridazin-3-yl)amino)pyridin-3-yl)piperazin-1-yl)-8-oxooctanoate

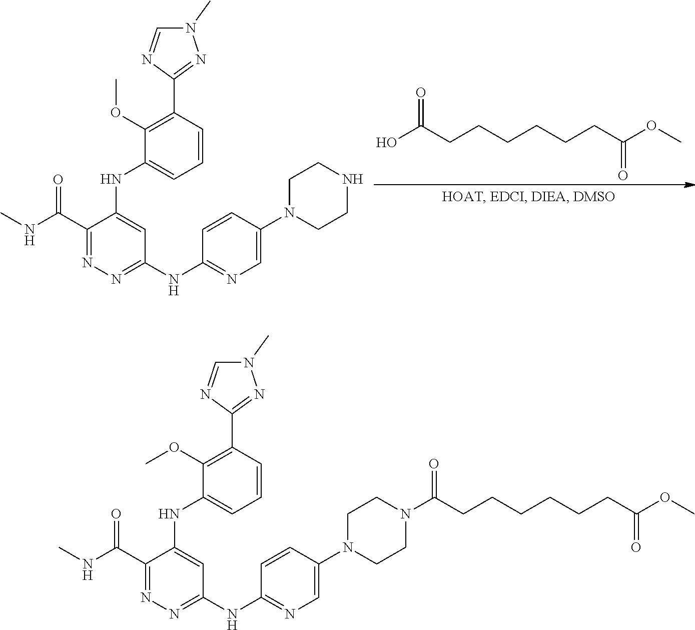

To a stirred solution of 4-((2-methoxy-3-(1-methyl-1H-1,2,4-triazol-3-yl)phenyl)amino)-N-methyl-6-((5-(piperidin-4-yl)pyridin-2-yl)amino)pyridazine-3-carboxamide (340 mg, 0.66 mmol) and 8-methoxy-8-oxooctanoic acid (186 mg, 0.99 mmol) in DMSO (10 mL) were added HOAT (224.4 mg, 1.65 mmol), EDCI (316.8 mg, 1.65 mmol) and DIEA (425.7 mg, 3.3 mmol). After the reaction mixture was stirred at rt overnight, it was poured into H₂O (100 mL) and extracted with ethyl acetate (100 mL×3). The combined organic layers were washed with brine, dried over Na₂SO₄, filtered, and concentrated under reduced pressure. The residue was purified by flash chromatography to give the title compound (385 mg, 85.4% yield) as a yellow solid. MS (ESI) m/z=685.6 [M+H]⁺.

### Step 2. Synthesis of 8-(4-(6-((5-((2-methoxy-3-(1-methyl-1H-1,2,4-triazol-3-yl)phenyl)amino)-6-(methylcarbamoyl)pyridazin-3-yl)amino)pyridin-3-yl)piperazin-1-yl)-8-oxooctanoic acid

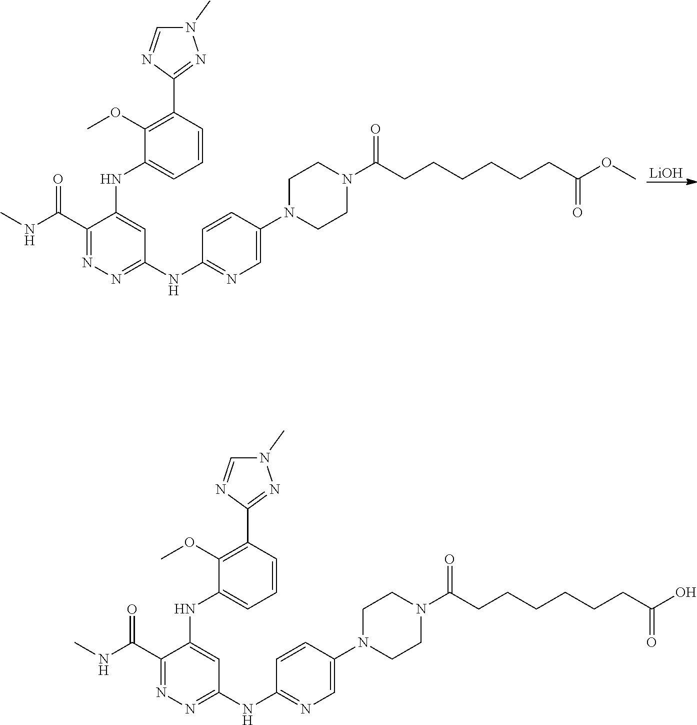

To a solution of methyl 8-(4-(6-((5-((2-methoxy-3-(1-methyl-1H-1,2,4-triazol-3-yl)phenyl)amino)-6-(methylcarbamoyl)pyridazin-3-yl)amino)pyridin-3-yl)piperidin-1-yl)-8-oxooctanoate (385 mg, 0.56 mmol) in THF (10 mL), MeOH (4 mL) and water (4 mL) was added LiOH (94.2 mg, 2.24 mmol) at 0° C. After the reaction mixture was stirred at rt for 2 h, it was purified by prep-HPLC to give the title compound (176 mg, 46.9% yield) as a yellow solid. MS (ESI) m/z=671.6 [M+H]⁺.

### Step 3. Synthesis of tert-butyl (2S,4R)-4-hydroxy-1-((S)-2-(8-(4-(6-((5-((2-methoxy-3-(1-methyl-1H-1,2,4-triazol-3-yl)phenyl)amino)-6-(methylcarbamoyl)pyridazin-3-yl)amino)pyridin-3-yl)piperazin-1-yl)-8-oxooctanamido)-3,3-dimethylbutanoyl)pyrrolidine-2-carboxylate

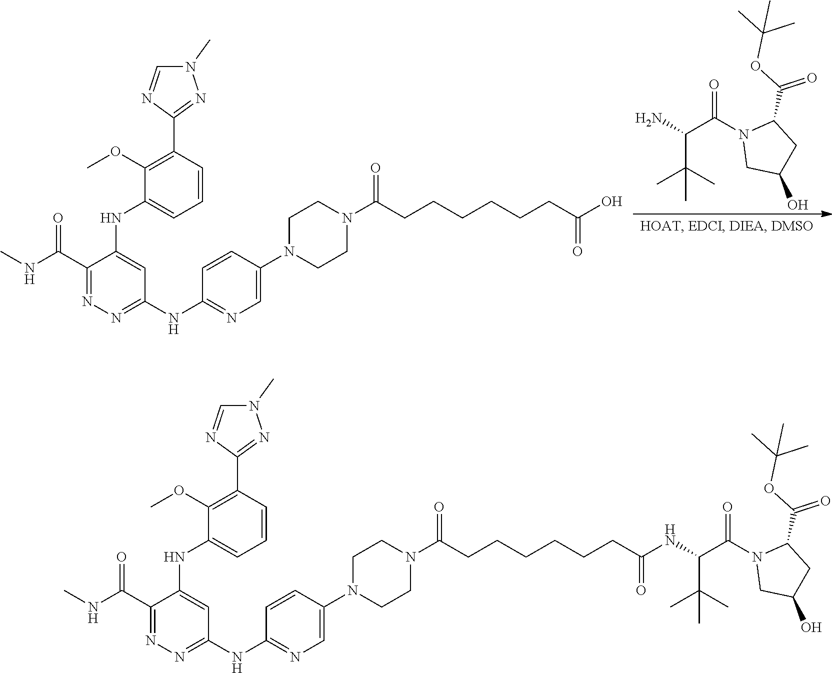 [SMILES: CNC(=O)c1nnc(Nc2ccc(N3CCN(C(=O)CCCCCCC(=O)O)CC3)cn2)cc1Nc1cccc(-c2ncn(C)n2)c1OC ; CC(C)(C)OC(=O)[C@@H]1C[C@@H](O)CN1C(=O)[C@@H](N)C(C)(C)C ; CNC(=O)c1nnc(Nc2ccc(N3CCN(C(=O)CCCCCCC(=O)N[C@H](C(=O)N4C[C@H](O)C[C@H]4C(=O)OC(C)(C)C)C(C)(C)C)CC3)cn2)cc1Nc1cccc(-c2ncn(C)n2)c1OC]

To a stirred solution of 8-(4-(6-((5-((2-methoxy-3-(1-methyl-1H-1,2,4-triazol-3-yl)phenyl)amino)-6-(methylcarbamoyl)pyridazin-3-yl)amino)pyridin-3-yl)piperidin-1-yl)-8-oxooctanoic acid (176 mg, 0.263 mmol) and tert-butyl (2S,4R)-1-((S)-2-amino-3,3-dimethylbutanoyl)-4-hydroxypyrrolidine-2-carboxylate (186 mg, 0.342 mmol) in DMSO (10 mL) were added HOAT (89.4 mg, 0.658 mmol) and EDCI (126.3 mg, 0.658 mmol) and DIEA (169.6 mg, 1.32 mmol). After the reaction mixture was stirred at rt overnight, it was poured into H₂O (100 mL) and extracted with ethyl acetate (100 mL×3). The combined organic layers were washed with brine, dried over Na₂SO₄, filtered, and concentrated under reduced pressure. The residue was purified by flash chromatography to give the title compound (165 mg, 73.7% yield) as a yellow solid. MS (ESI) m/z=954.0 [M+H]⁺.

### Step 4. Synthesis of (2S,4R)-4-hydroxy-1-((S)-2-(8-(4-(6-((5-((2-methoxy-3-(1-methyl-1H-1,2,4-triazol-3-yl)phenyl)amino)-6-(methylcarbamoyl)pyridazin-3-yl)amino)pyridin-3-yl)piperazin-1-yl)-8-oxooctanamido)-3,3-dimethylbutanoyl)pyrrolidine-2-carboxylic acid

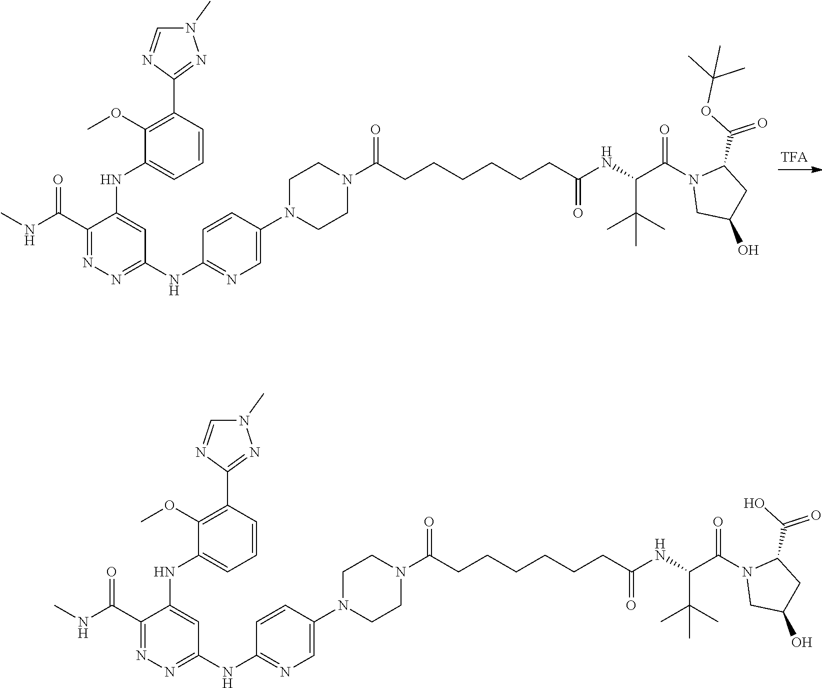 [SMILES: CNC(=O)c1nnc(Nc2ccc(N3CCN(C(=O)CCCCCCC(=O)N[C@H](C(=O)N4C[C@H](O)C[C@H]4C(=O)OC(C)(C)C)C(C)(C)C)CC3)cn2)cc1Nc1cccc(-c2ncn(C)n2)c1OC ; CNC(=O)c1nnc(Nc2ccc(N3CCN(C(=O)CCCCCCC(=O)N[C@H](C(=O)N4C[C@H](O)C[C@H]4C(=O)O)C(C)(C)C)CC3)cn2)cc1Nc1cccc(-c2ncn(C)n2)c1OC]

To a solution of tert-butyl (2S,4R)-4-hydroxy-1-((S)-2-(8-(4-(6-((5-((2-methoxy-3-(1-methyl-1H-1,2,4-triazol-3-yl)phenyl)amino)-6-(methylcarbamoyl)pyridazin-3-yl)amino)pyridin-3-yl)piperazin-1-yl)-8-oxooctanamido)-3,3-dimethylbutanoyl)pyrrolidine-2-carboxylate (165 mg, 0.173 mmol) in DCM (5 mL) was added TFA (5 mL). After the reaction mixture was stirred at rt for 2 h, it was concentrated to give the title compound (145 mg, 93% yield) which was used directly in the next step without further purification. MS (ESI) m/z=898.0 [M+H]⁺.

### Step 5. Synthesis of 6-((5-(1-(8-(((S)-1-((2S,4R)-2-((4-cyanobenzyl)carbamoyl)-4-hydroxypyrrolidin-1-yl)-3,3-dimethyl-1-oxobutan-2-yl)amino)-8-oxooctanoyl)piperidin-4-yl)pyridin-2-yl)amino)-4-((2-methoxy-3-(1-methyl-1H-1,2,4-triazol-3-yl)phenyl)amino)-N-methylpyridazine-3-carboxamide

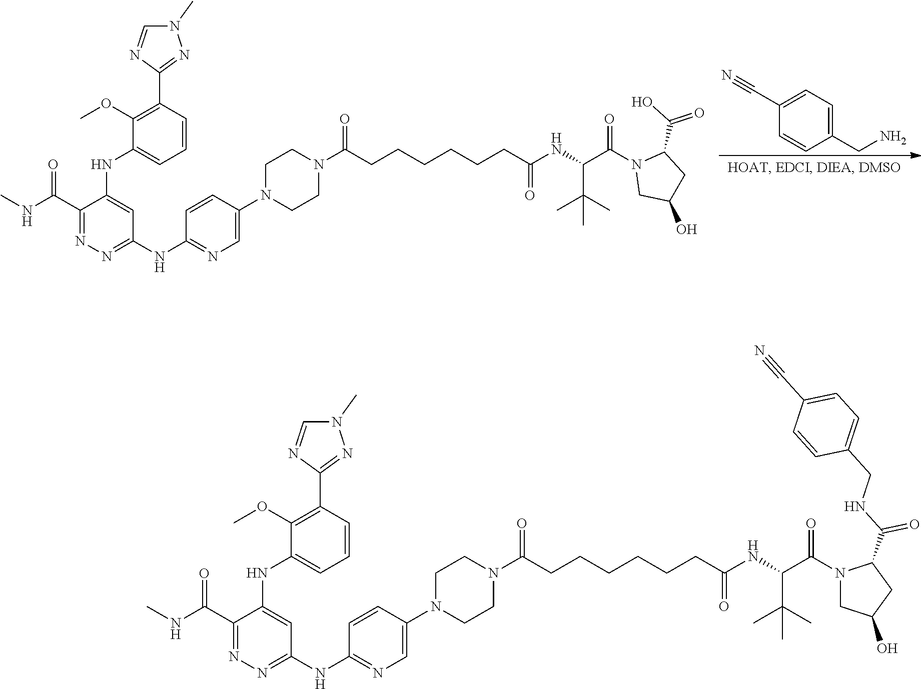 [SMILES: CNC(=O)c1nnc(Nc2ccc(N3CCN(C(=O)CCCCCCC(=O)N[C@H](C(=O)N4C[C@H](O)C[C@H]4C(=O)O)C(C)(C)C)CC3)cn2)cc1Nc1cccc(-c2ncn(C)n2)c1OC ; N#Cc1ccc(CN)cc1 ; CNC(=O)c1nnc(Nc2ccc(N3CCN(C(=O)CCCCCCC(=O)N[C@H](C(=O)N4C[C@H](O)C[C@H]4C(=O)NCc4ccc(C#N)cc4)C(C)(C)C)CC3)cn2)cc1Nc1cccc(-c2ncn(C)n2)c1OC]

To a stirred solution of (2S,4R)-4-hydroxy-1-((S)-2-(8-(4-(6-((5-((2-methoxy-3-(1-methyl-1H-1,2,4-triazol-3-yl)phenyl)amino)-6-(methylcarbamoyl)pyridazin-3-yl)amino)pyridin-3-yl)piperazin-1-yl)-8-oxooctanamido)-3,3-dimethylbutanoyl)pyrrolidine-2-carboxylic acid (8 mg, 8.9 μmol) and 4-(aminomethyl)benzonitrile (1.76 mg, 13.4 mmol) in DMSO (2.5 mL) were added HOAT (3.0 mg, 22.3 μmol) and EDCI (4.28 mg, 22.3 mmol) and DIEA (5.8 mg, 44.9 μmol). After the reaction mixture was stirred at rt overnight, it was poured into H₂O (100 mL) and extracted with ethyl acetate (100 mL×3). The combined organic layers was washed with brine, dried over Na₂SO₄, filtered, and concentrated under reduced pressure. The residue was purified by flash chromatography to give the title compound (3.2 mg, 35.5% yield) as a yellow solid. MS (ESI) m/z=1013.0 [M+H]⁺.

### Example 234: 6-((5-(4-(8-(((S)-1-((2S,4R)-2-(((5-Cyanopyridin-2-yl)methyl)carbamoyl)-4-hydroxypyrrolidin-1-yl)-3,3-dimethyl-1-oxobutan-2-yl)amino)-8-oxooctanoyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-172)

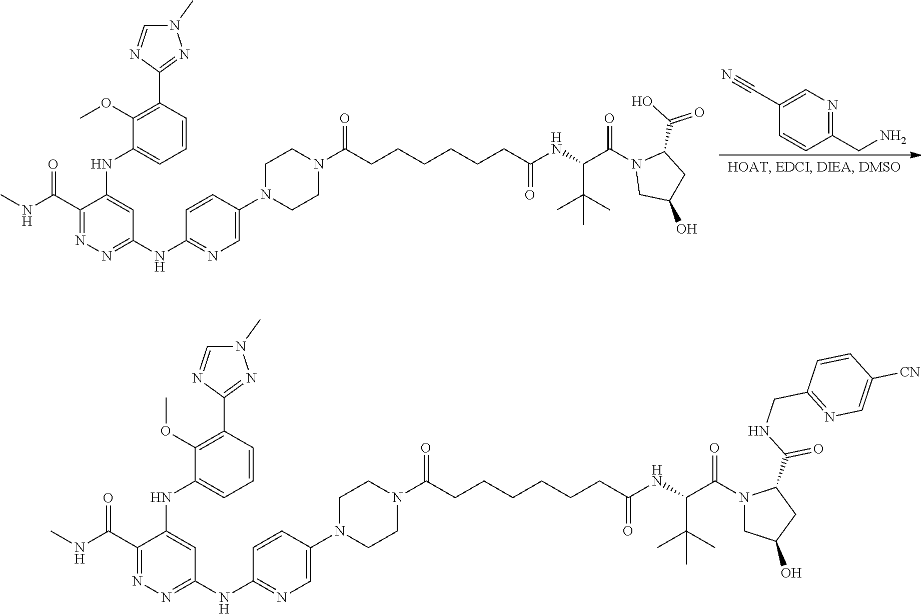 [SMILES: CNC(=O)c1nnc(Nc2ccc(N3CCN(C(=O)CCCCCCC(=O)N[C@H](C(=O)N4C[C@H](O)C[C@H]4C(=O)O)C(C)(C)C)CC3)cn2)cc1Nc1cccc(-c2ncn(C)n2)c1OC ; N#Cc1ccc(CN)nc1 ; CNC(=O)c1nnc(Nc2ccc(N3CCN(C(=O)CCCCCCC(=O)N[C@H](C(=O)N4C[C@H](O)C[C@H]4C(=O)NCc4ccc(C#N)cn4)C(C)(C)C)CC3)cn2)cc1Nc1cccc(-c2ncn(C)n2)c1OC]

CPD-172 was synthesized following the same procedure for preparing CPD-171 (1.5 mg, yield: 16.2%). MS (ESI) m/z=1013.9 [M+H]⁺.

### Example 235: 6-((5-(4-(8-(((S)-1-((2S,4R)-2-(((S)-2-Cyano-1-methyl-4,5,6,7-tetrahydro-1H-indol-4-yl)carbamoyl)-4-hydroxypyrrolidin-1-yl)-3,3-dimethyl-1-oxobutan-2-yl)amino)-8-oxooctanoyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-173)

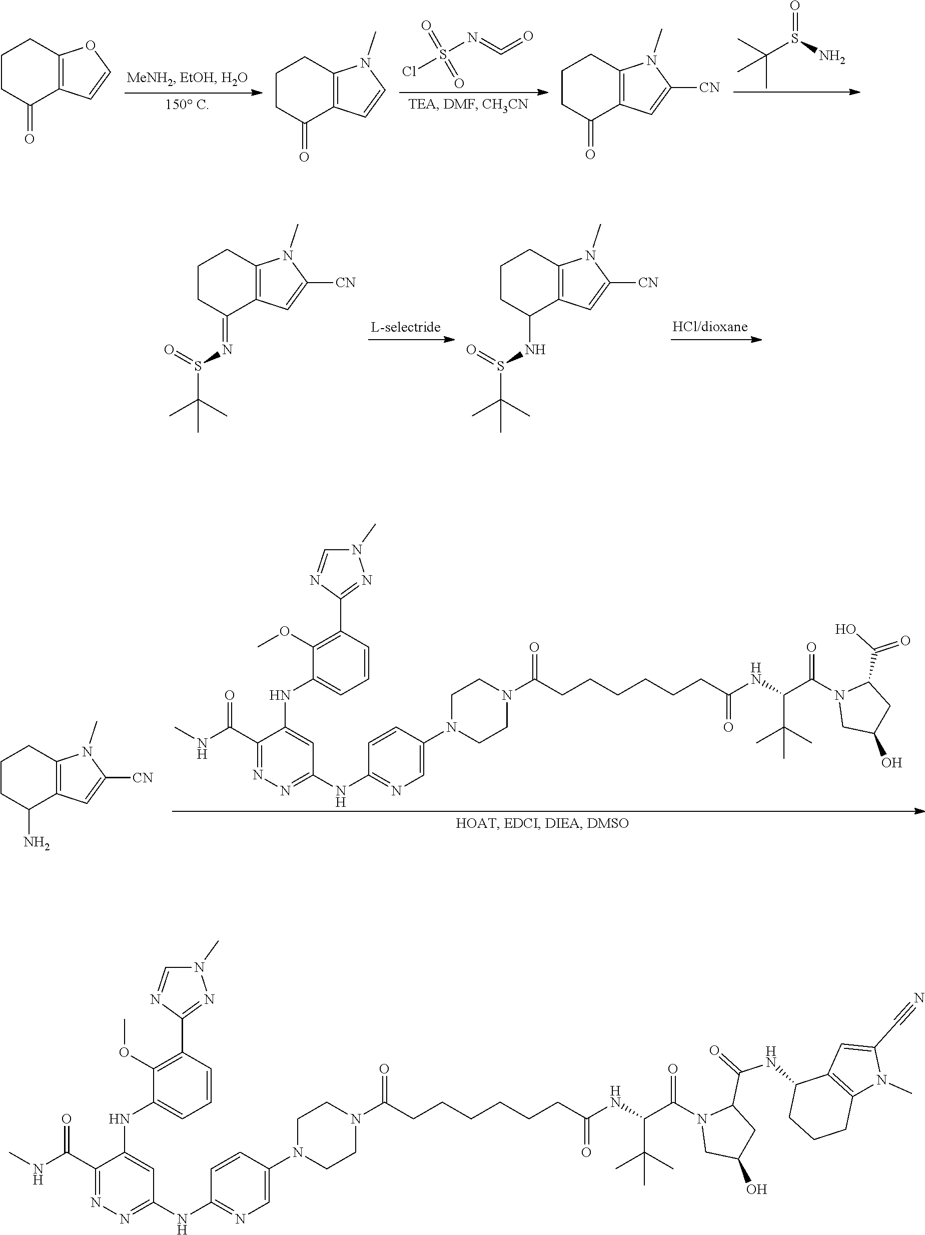

### Step 1. Synthesis of 1-methyl-1,5,6,7-tetrahydro-4H-indol-4-one

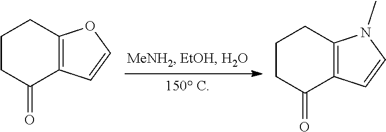

A mixture of 6,7-dihydrobenzofuran-4(5H)-one (5 g, 36.7 mmol) in CH₃NH₂/EtOH (30%, 15 mL) and H₂O (60 mL) was heated in a scaled tube at 150° C. for 16 h. The mixture was poured into water and extracted with DCM (30 mL×3). The combined organic phase was dried over Na₂SO₄, filtered, and concentrated under reduced pressure. The residue was purified by silica gel column chromatography (petroleum ether/EtOAc=1:1) to give the title compound (4.7 g, 87% yield) as a yellow solid. MS (ESI) m/z=150.1 [M+H]⁺.

### Step 2. Synthesis of 1-methyl-4-oxo-4,5,6,7-tetrahydro-1H-indole-2-carbonitrile

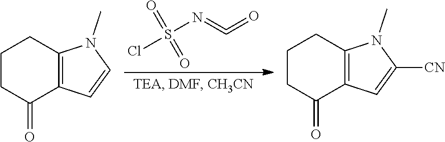

To a solution of 1-methyl-1,5,6,7-tetrahydro-4H-indol-4-one (3.48 g, 23.3 mmol) in CH₃CN (10 mL) was added sulfurisocyanatidic chloride in CH₃CN (5 mL) dropwise at 0° C. The mixture was stirred at 0° C. for 15 min, before DMF (3.4 g, 46.6 mmol) and TEA (4.7 g, 46.6 mmol) were added. After the resulting mixture was stirred at 10° C. for 1 h, it was filtered. The filtrate was diluted with water and extracted with ethyl acetate (20 mL×3). The combined organic phase was washed with brine, dried over Na₂SO₄, filtered, and concentrated under reduced pressure. The residue was purified by prep-TLC (petroleum ether/EtOAc=5:1) to give the title compound (3.7 mg, 91% yield) as a yellow solid. MS (ESI) m/z=175.1 [M+H]⁺.

### Step 3. Synthesis of (R,E)-N-(2-cyano-1-methyl-1,5,6,7-tetrahydro-4H-indol-4-ylidene)-2-methylpropane-2-sulfinamide

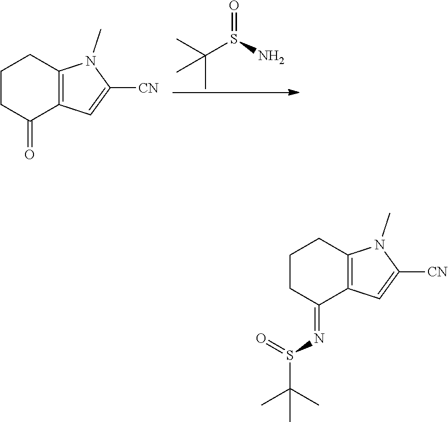

To a mixture of 1-methyl-4-oxo-4,5,6,7-tetrahydro-1H-indole-2-carbonitrile (3.9 g, 22.4 mmol) and (R)-2-methylpropane-2-sulfinamide (2.7 g, 22.4 mmol) in THF (60 mL) was added titanium tetraisopropanolate (12.7 g, 44.8 mmol) at 0° C. After the mixture was stirred at 80° C. for 16 h, it was diluted with water, filtered and extracted with EtOAc (8 mL×3). The combined organic phase was washed with brine, dried over Na₂SO₄, filtered, and concentrated under reduced pressure. The residue was purified by prep-TLC (petroleum ether/EtOAc=3:1) to give the title compound (1.1 g, 18% yield) as a yellow solid. MS (ESI) m/z=278.1 [M+H]⁺.

### Step 4. Synthesis of (R)—N—((S)-2-cyano-1-methyl-4,5,6,7-tetrahydro-1H-indol-4-yl)-2-methylpropane-2-sulfinamide

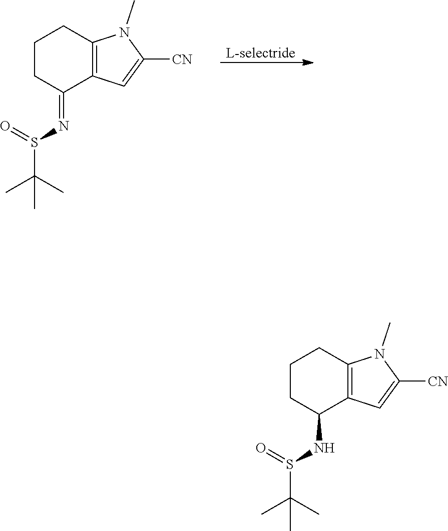 [SMILES: Cn1c(C#N)cc2c1CCC/C2=N\[S@](=O)C(C)(C)C ; "L-selectride" ; Cn1c(C#N)cc2c1CCC[C@@H]2N[S@](=O)C(C)(C)C]

To a solution of (R,E)-N-(2-cyano-1-methyl-1,5,6,7-tetrahydro-4H-indol-4-ylidene)-2-methylpropane-2-sulfinamide (1 g, 3.6 mmol) in THE (10 mL) was added L-selectride (1M, 7.2 mL, 7.2 mmol) dropwise at 0° C. After the mixture was stirred at rt for 6 h, it was quenched with MeOH and concentrated under reduced pressure. The residue was purified by prep-TLC (petroleum ether/EtOAc=1:1) to give the title compound (300 mg, 30% yield) as a yellow solid. MS (ESI) m/z=280.1 [M+H]⁺.

### Step 5. Synthesis of (S)-4-amino-1-methyl-4,5,6,7-tetrahydro-1H-indole-2-carbonitrile

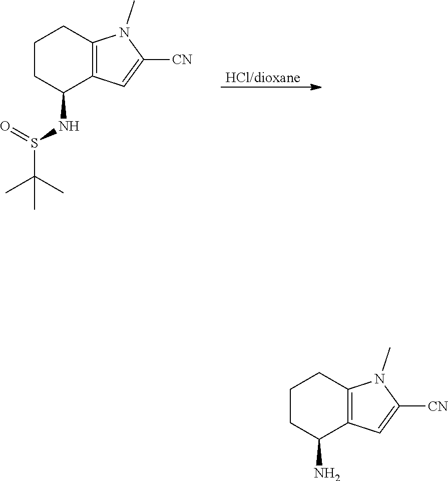 [SMILES: Cn1c(C#N)cc2c1CCC[C@@H]2N[S@](=O)C(C)(C)C ; "HCl/dioxane" ; Cn1c(C#N)cc2c1CCC[C@@H]2N]

To a solution of (R)—N—((S)-2-cyano-1-methyl-4,5,6,7-tetrahydro-1H-indol-4-yl)-2-methylpropane-2-sulfinamide (300 mg, 1.07 mmol) in MeOH (3 mL) was added HCl/dioxane (3 M, 3 mL). After the mixture was stirred at it for 3 h, it was concentrated under reduced pressure. The residue was purified by prep-HPLC to give the title compound (130.3 mg, 69% yield) as a yellow solid. ¹H NMR (400 MHz, DMSO-d₆) δ 6.84 (s, 1H), 3.68-3.53 (m, 1H), 2.51 (s, 3H), 1.96-1.82 (m, 4H), 1.65-1.62 (m, 1H), 1.33-1.29 (m, 1H). MS (ESI) m/z=176.1 [M+H]⁺.

### Step 6. Synthesis of 6-((5-(4-(8-(((S)-1-((2S,4R)-2-(((S)-2-cyano-1-methyl-4,5,6,7-tetrahydro-1H-indol-4-yl)carbamoyl)-4-hydroxypyrrolidin-1-yl)-3,3-dimethyl-1-oxobutan-2-yl)amino)-8-oxooctanoyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide

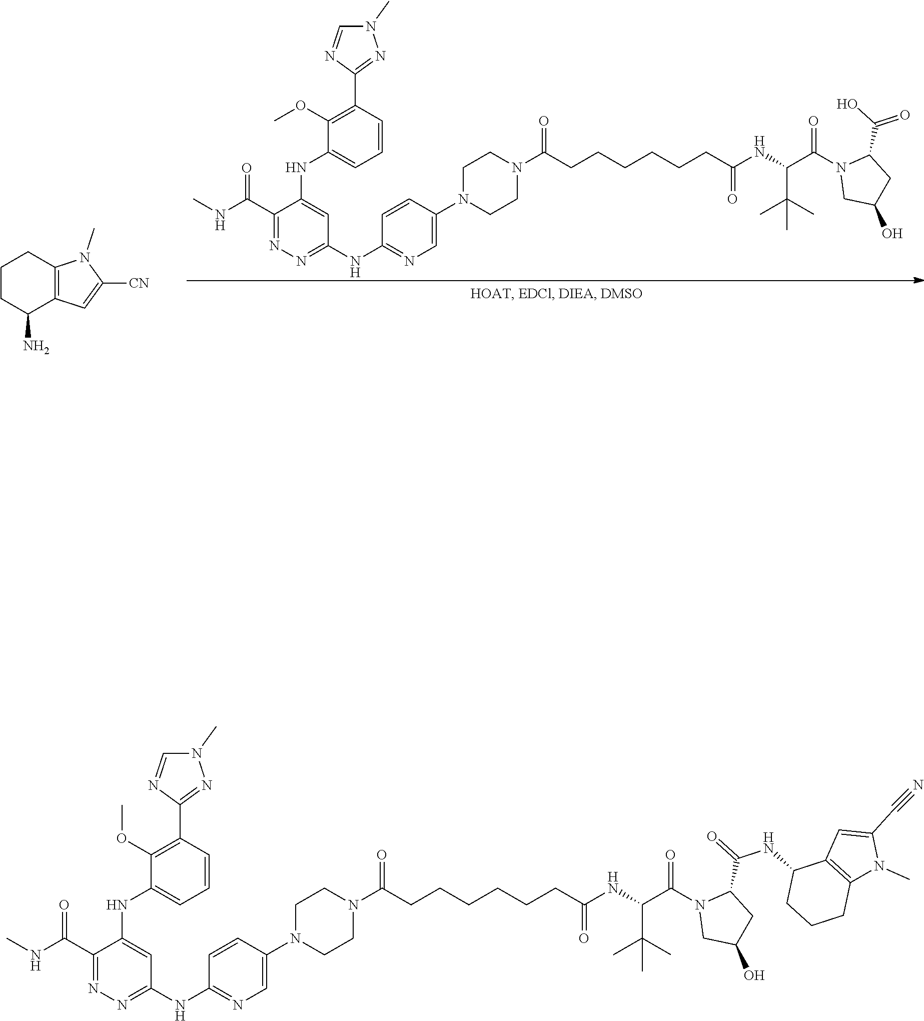 [SMILES: Cn1c(C#N)cc2c1CCC[C@@H]2N ; CNC(=O)c1nnc(Nc2ccc(N3CCN(C(=O)CCCCCCC(=O)N[C@H](C(=O)N4C[C@H](O)C[C@H]4C(=O)O)C(C)(C)C)CC3)cn2)cc1Nc1cccc(-c2ncn(C)n2)c1OC ; "HOAT, EDCl, DIEA, DMSO" ; CNC(=O)c1nnc(Nc2ccc(N3CCN(C(=O)CCCCCCC(=O)N[C@H](C(=O)N4C[C@H](O)C[C@H]4C(=O)N[C@H]4CCCc5c4cc(C#N)n5C)C(C)(C)C)CC3)cn2)cc1Nc1cccc(-c2ncn(C)n2)c1OC]

CPD-173 was synthesized following the same procedure for preparing CPD-171 (1.6 mg, yield: 16.5%). MS (ESI) m/z=1056.2 [M+H]⁺.

### Example 236: 6-((5-(4-(8-(((S)-1-((2S,4R)-4-Hydroxy-2-(((S)-3-methyl-2-oxo-1,2,5,6,7,8-hexahydroquinolin-5-yl)carbamoyl)pyrrolidin-1-yl)-3,3-dimethyl-1-oxobutan-2-yl)amino)-8-oxooctanoyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-174)

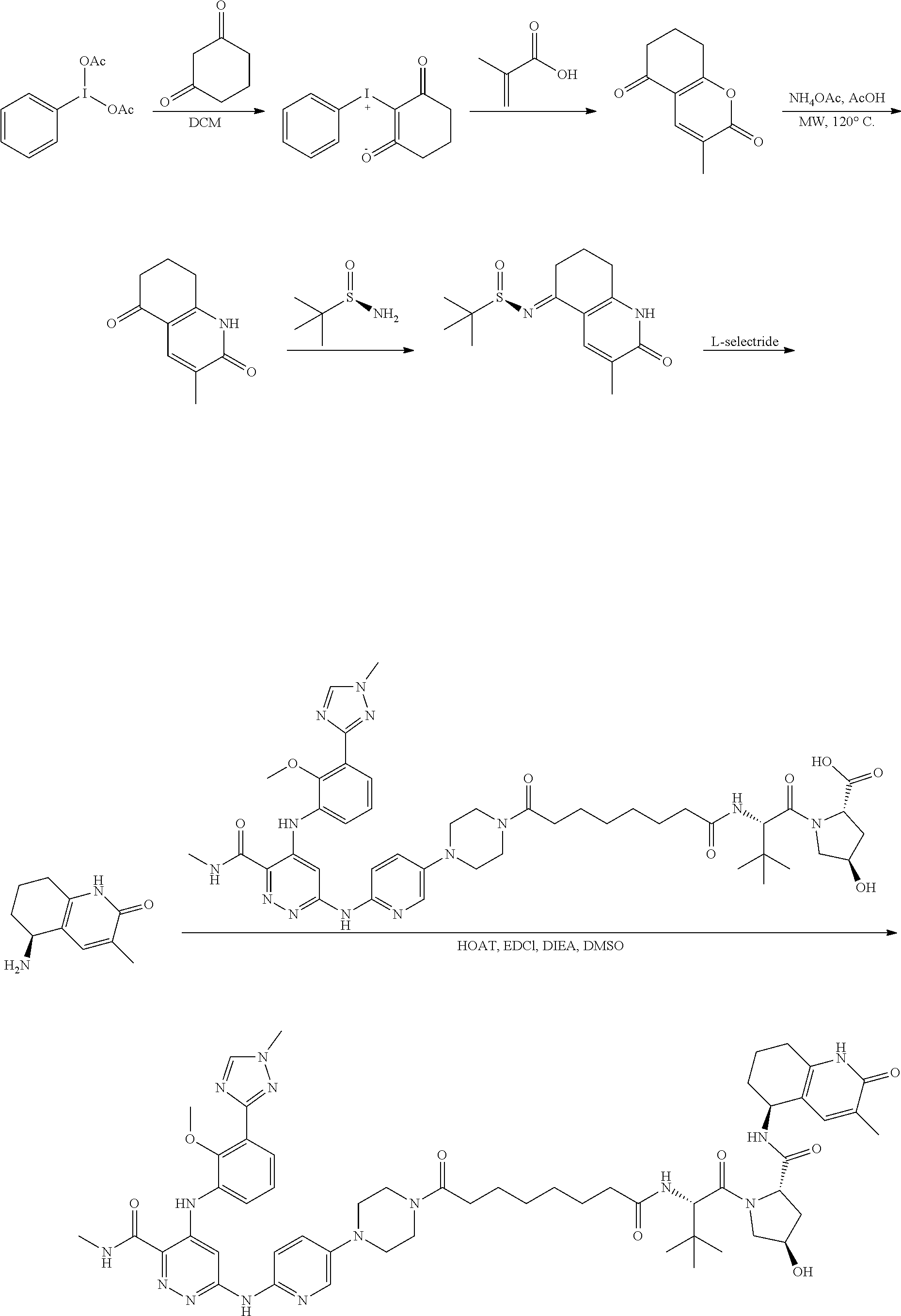

### Step 1. Synthesis of 3-oxo-2-(phenyliodonio)cyclohex-1-en-1-olate

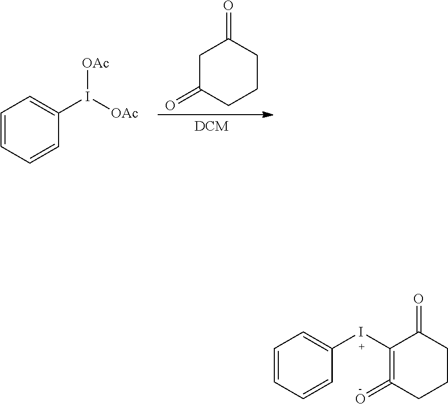

A mixture of phenyl-λ³-iodanediyl diacetate (5 g, 15.5 mmol) and cyclohexane-1,3-dionein (1.74 g, 15.5 mmol) in DCM (100 mL) was stirred at rt for 16 h. The reaction mixture was washed with 5% KOH, water, dried over Na₂SO₄, filtered, and concentrated under reduced pressure. The residue was triturated with hexanes and filtered to give the title compound (1.67 g, 34% yield) as a yellow solid. MS (ESI) m/z=314.1 [M+H]⁺.

### Step 2. Synthesis of 3-methyl-7,8-dihydro-2H-chromene-2,5(6H)-dione

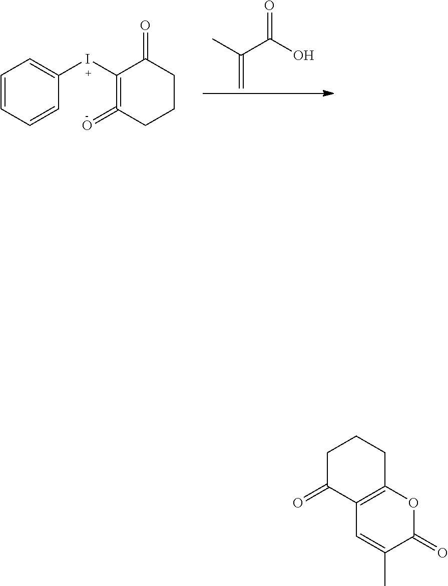

A mixture of 3-oxo-2-(phenyliodonio)cyclohex-1-en-1-olate (1.57 g, 5 mmol), methacrylic acid (430.3 mg, 5 mmol), [Cp*RhCl2]₂ (309 mg, 0.5 mmol), NaOAc (102.5 mg, 1.25 mmol) and HFIP (30 mL) was stirred at 80° C. for 16 h. The reaction mixture was concentrated under reduced pressure. The residue was purified by silica gel column chromatography (petroleum ether/EtOAc=10:1) to give the title compound (685 mg, 77% yield) as a white solid. MS (ESI) m/z=179.1 [M+H]⁺.

### Step 3. Synthesis of 3-methyl-1-dihydroquinoline-2,5(1H,6H)-dione

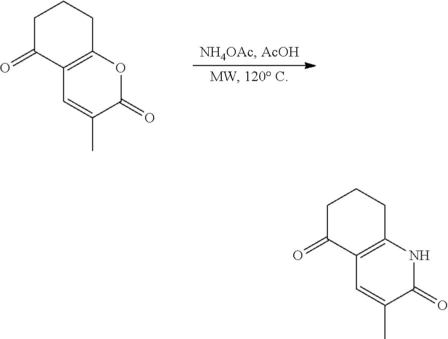

A mixture of 3-methyl-7,8-dihydro-2H-chromene-2,5(6H)-dione (585 mg, 3.28 mmol) and NH₄OAc (506 mg, 6.57 mmol) in AcOH (3 mL) was stirred at 120° C. under microwave irradiation for 2 h. After the pH of the mixture was adjusted to 8˜9 with aq. NaHCO₃ it was extracted with EtOAc (5 mL×3). The organic phase was dried over Na₂SO₄, filtered, and concentrated to give the title compound (355 mg, 61% yield) as a yellow solid. MS (ESI) m/z=178.1 [M+H]⁺.

### Step 4 to step 6. Synthesis of 6-((5-(4-(8-(((S)-1-((2S,4R)-4-hydroxy-2-(((S)-3-methyl-2-oxo-1,2,5,6,7,8-hexahydroquinolin-5-yl)carbamoyl)pyrrolidin-1-yl)-3,3-dimethyl-1-oxobutan-2-yl)amino)-8-oxooctanoyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide

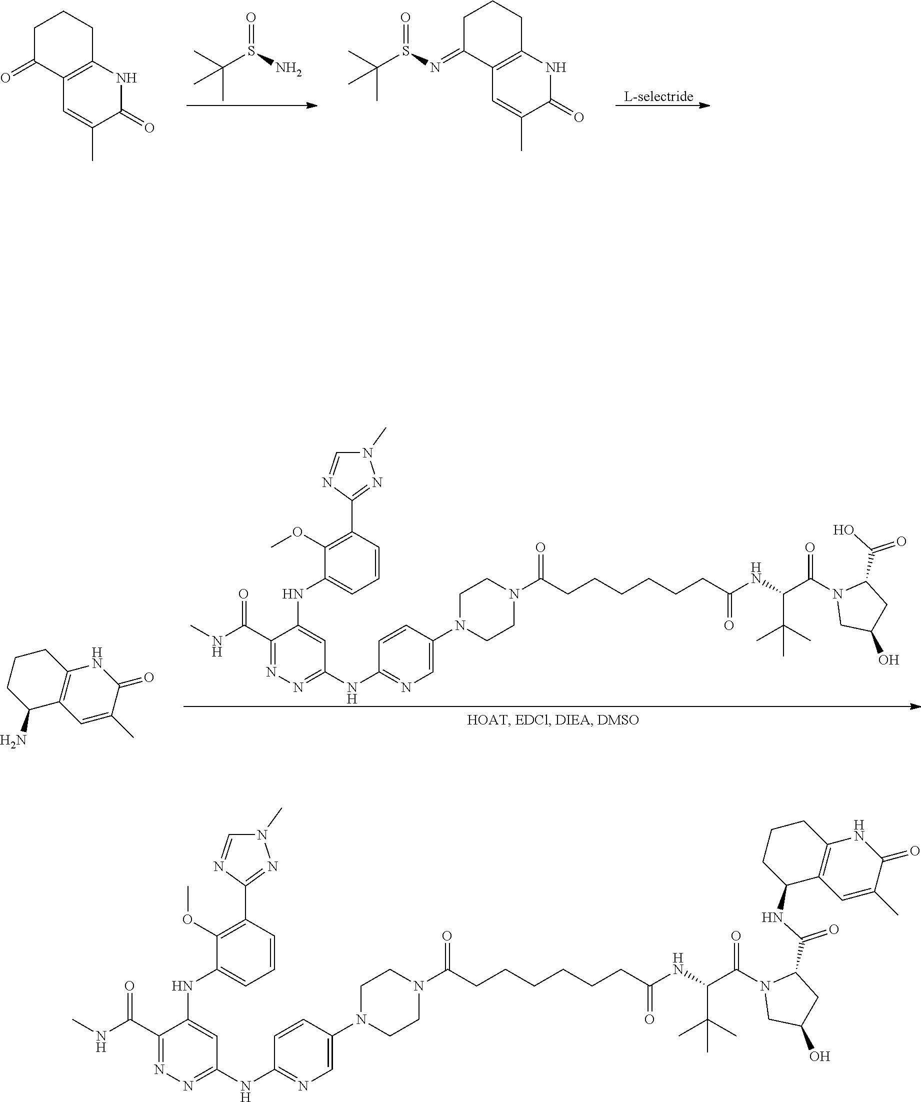

CPD-174 was synthesized following the same procedures for preparing CPD-171 (5.4 mg, yield: 91%). MS (ESI) m/z=1059.2 [M+H]⁺.

### Example 237: 6-((5-((3-((2-(2,6-Dioxopiperidin-3-yl)-1,3-dioxoisoindolin-5-yl)amino)propyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-175)

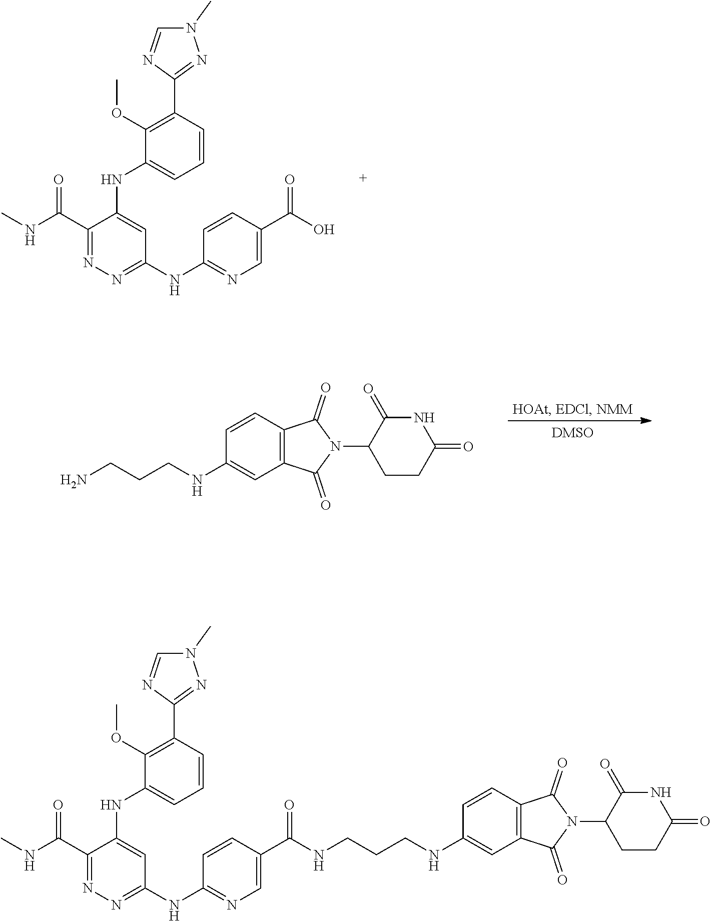

CPD-175 was synthesized following the same procedure for preparing CPD-137 (5.5 mg, yield: 15.8%). MS (ESI) m/z=788.8 [M+H]⁺.

### Example 238: 6-((5-((2-((2-(2,6-Dioxopiperidin-3-yl)-1,3-dioxoisoindolin-5-yl)amino)ethyl)carbamoyl)pyridin-2-yl)amino)-4-((2-methoxy-3-(1-methyl-1H-1,2,4-triazol-3-yl)phenyl)amino)-N-methylpyridazine-3-carboxamide CPD-176)

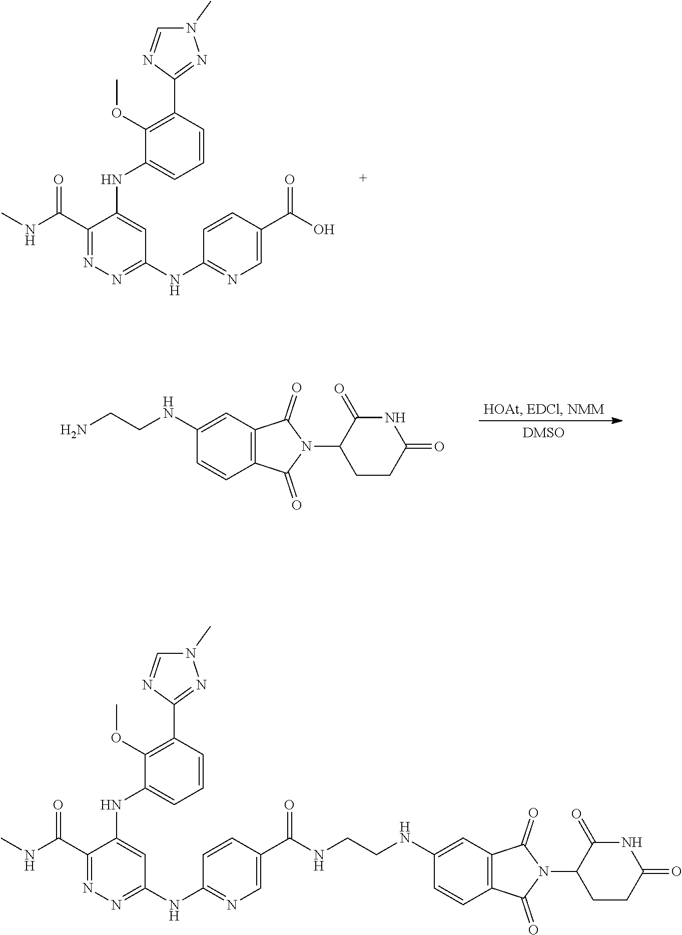

CPD-176 was synthesized following the same procedure for preparing CPD-137 (11 mg, yield: 24.1%). MS (ESI) m/z=774.8 [M+H]⁺.

Certain compounds disclosed herein have the structures shown in Table 1.

TABLE 1

| Cpd. | Structure |
|---|---|
| CPD- 001 | 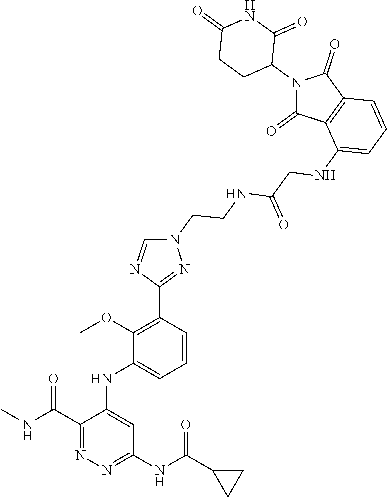 [SMILES: CNC(=O)c1nnc(NC(=O)C2CC2)cc1Nc1cccc(-c2ncn(CCNC(=O)CNc3cccc4c3C(=O)N(C3CCC(=O)NC3=O)C4=O)n2)c1OC] |
| CPD- 002 | 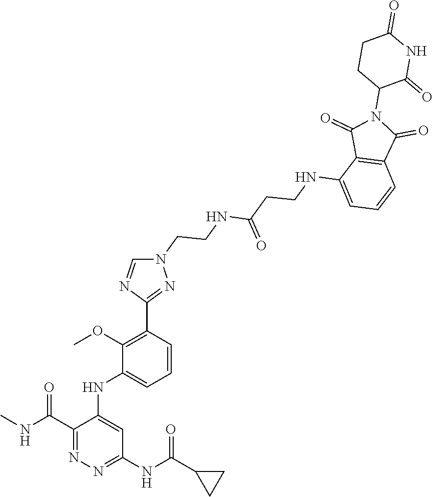 [SMILES: CNC(=O)c1nnc(NC(=O)C2CC2)cc1Nc1cccc(-c2ncn(CCNC(=O)CCNc3cccc4c3C(=O)N(C3CCC(=O)NC3=O)C4=O)n2)c1OC] |
| CPD- 003 | 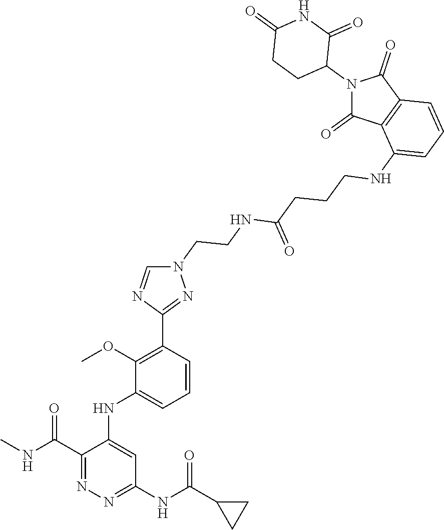 [SMILES: CNC(=O)c1nnc(NC(=O)C2CC2)cc1Nc1cccc(-c2ncn(CCNC(=O)CCCNc3cccc4c3C(=O)N(C3CCC(=O)NC3=O)C4=O)n2)c1OC] |
| CPD- 004 | 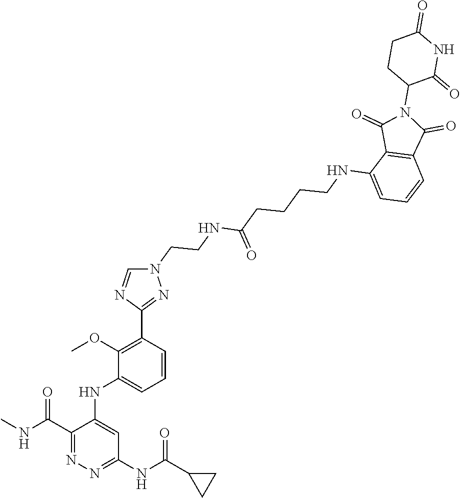 [SMILES: CNC(=O)c1nnc(NC(=O)C2CC2)cc1Nc1cccc(-c2ncn(CCNC(=O)CCCCNc3cccc4c3C(=O)N(C3CCC(=O)NC3=O)C4=O)n2)c1OC] |
| CPD- 005 | 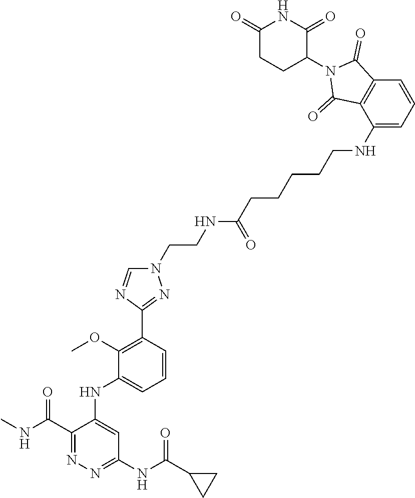 [SMILES: CNC(=O)c1nnc(NC(=O)C2CC2)cc1Nc1cccc(-c2ncn(CCNC(=O)CCCCCNc3cccc4c3C(=O)N(C3CCC(=O)NC3=O)C4=O)n2)c1OC] |
| CPD- 006 | 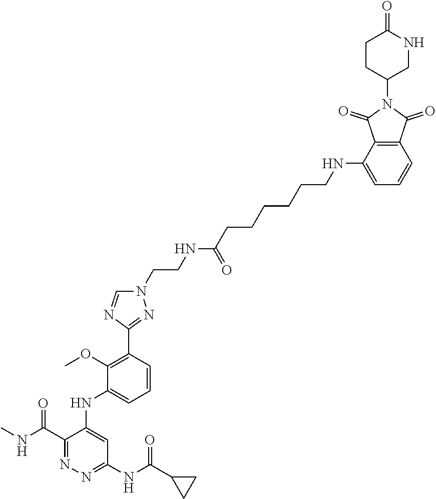 [SMILES: CNC(=O)c1nnc(NC(=O)C2CC2)cc1Nc1cccc(-c2ncn(CCNC(=O)CCCCCCNc3cccc4c3C(=O)N(C3CCC(=O)NC3)C4=O)n2)c1OC] |
| CPD- 007 | 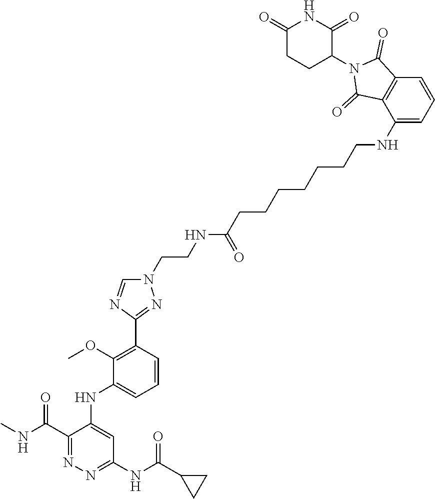 [SMILES: CNC(=O)c1nnc(NC(=O)C2CC2)cc1Nc1cccc(-c2ncn(CCNC(=O)CCCCCCCNc3cccc4c3C(=O)N(C3CCC(=O)NC3=O)C4=O)n2)c1OC] |
| CPD- 008 | 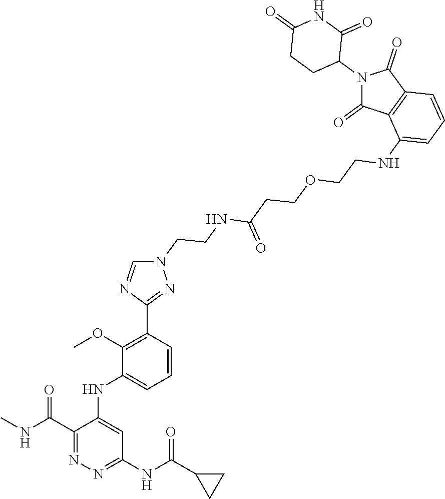 [SMILES: CNC(=O)c1nnc(NC(=O)C2CC2)cc1Nc1cccc(-c2ncn(CCNC(=O)CCOCCNc3cccc4c3C(=O)N(C3CCC(=O)NC3=O)C4=O)n2)c1OC] |
| CPD- 009 | 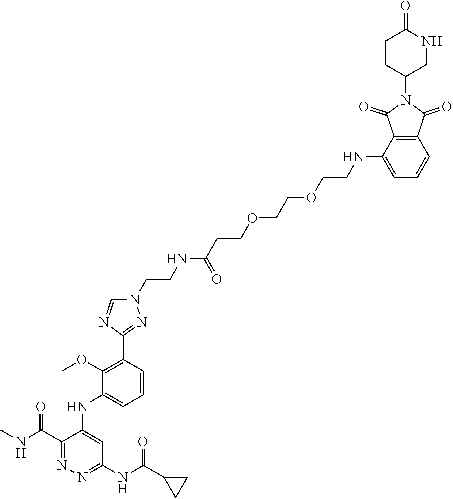 [SMILES: CNC(=O)c1nnc(NC(=O)C2CC2)cc1Nc1cccc(-c2ncn(CCNC(=O)CCOCCOCCNc3cccc4c3C(=O)N(C3CCC(=O)NC3)C4=O)n2)c1OC] |
| CPD- 010 | 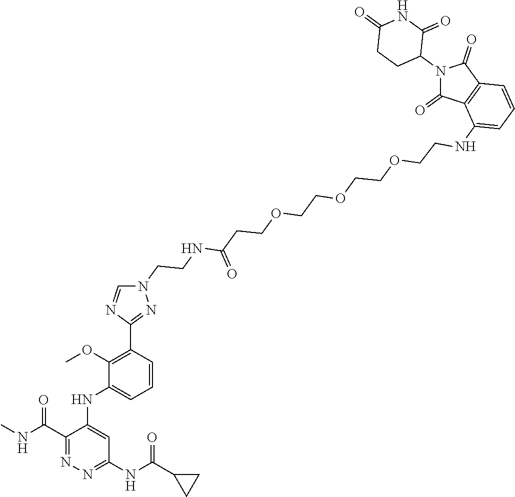 [SMILES: CNC(=O)c1nnc(NC(=O)C2CC2)cc1Nc1cccc(-c2ncn(CCNC(=O)CCOCCOCCOCCNc3cccc4c3C(=O)N(C3CCC(=O)NC3=O)C4=O)n2)c1OC] |
| CPD- 011 | 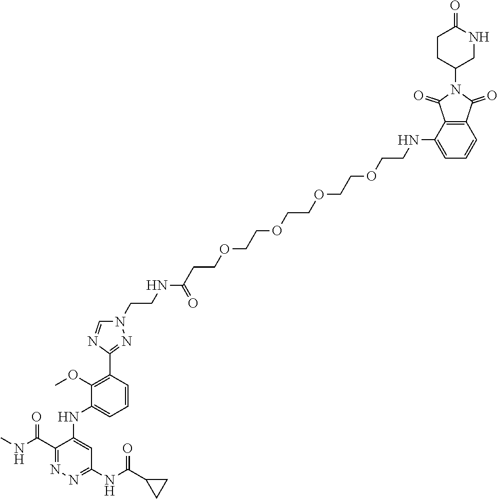 [SMILES: CNC(=O)c1nnc(NC(=O)C2CC2)cc1Nc1cccc(-c2ncn(CCNC(=O)CCOCCOCCOCCOCCNc3cccc4c3C(=O)N(C3CCC(=O)NC3)C4=O)n2)c1OC] |
| CPD- 012 | 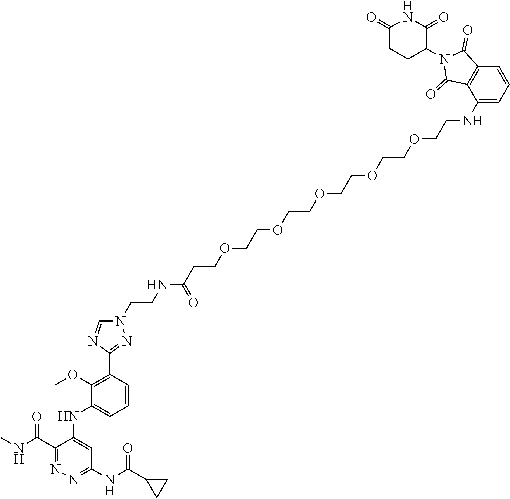 [SMILES: CNC(=O)c1nnc(NC(=O)C2CC2)cc1Nc1cccc(-c2ncn(CCNC(=O)CCOCCOCCOCCOCCOCCNc3cccc4c3C(=O)N(C3CCC(=O)NC3=O)C4=O)n2)c1OC] |
| CPD- 013 | 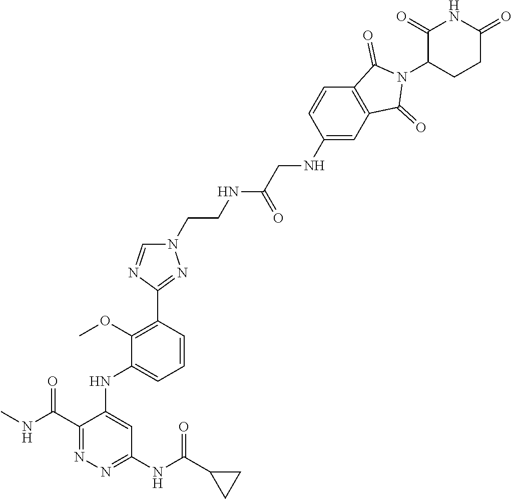 [SMILES: CNC(=O)c1nnc(NC(=O)C2CC2)cc1Nc1cccc(-c2ncn(CCNC(=O)CNc3ccc4c(c3)C(=O)N(C3CCC(=O)NC3=O)C4=O)n2)c1OC] |
| CPD- 014 | 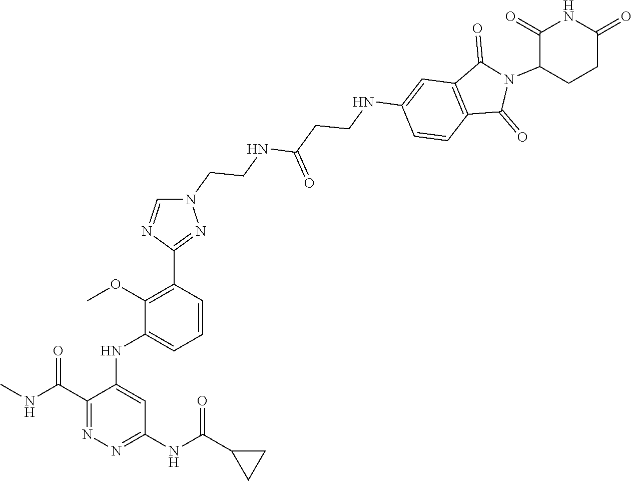 [SMILES: CNC(=O)c1nnc(NC(=O)C2CC2)cc1Nc1cccc(-c2ncn(CCNC(=O)CCNc3ccc4c(c3)C(=O)N(C3CCC(=O)NC3=O)C4=O)n2)c1OC] |
| CPD- 015 | 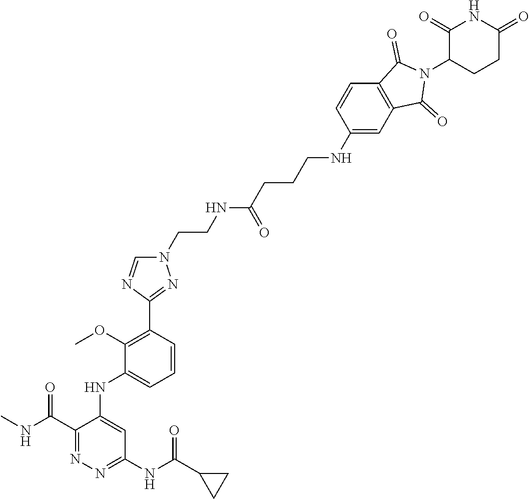 [SMILES: CNC(=O)c1nnc(NC(=O)C2CC2)cc1Nc1cccc(-c2ncn(CCNC(=O)CCCNc3ccc4c(c3)C(=O)N(C3CCC(=O)NC3=O)C4=O)n2)c1OC] |
| CPD- 016 | 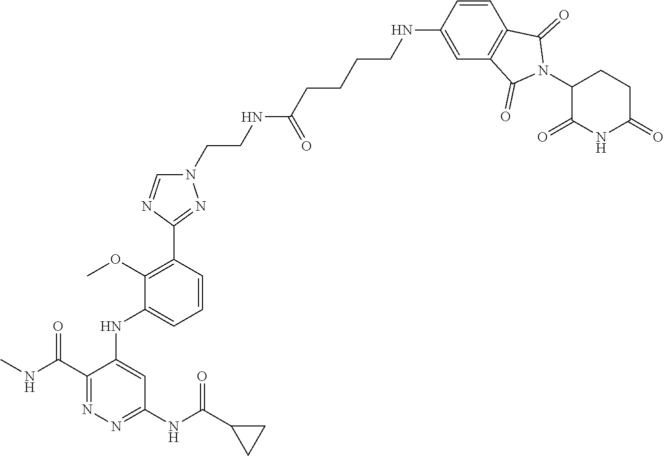 [SMILES: CNC(=O)c1nnc(NC(=O)C2CC2)cc1Nc1cccc(-c2ncn(CCNC(=O)CCCCNc3ccc4c(c3)C(=O)N(C3CCC(=O)NC3=O)C4=O)n2)c1OC] |
| CPD- 017 | 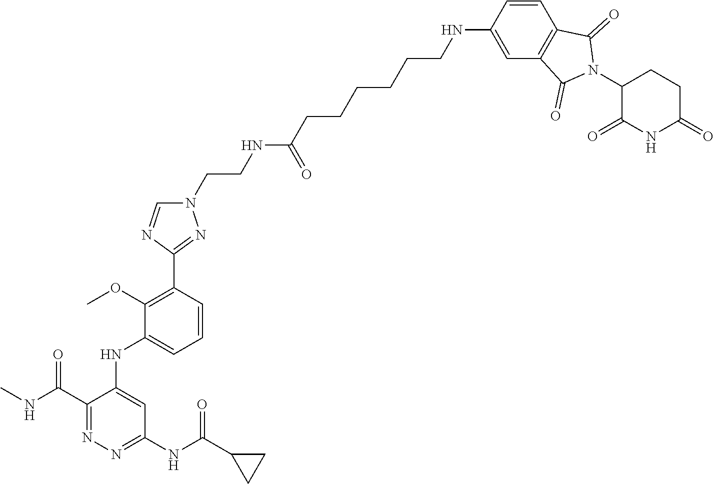 [SMILES: CNC(=O)c1nnc(NC(=O)C2CC2)cc1Nc1cccc(-c2ncn(CCNC(=O)CCCCCCNc3ccc4c(c3)C(=O)N(C3CCC(=O)NC3=O)C4=O)n2)c1OC] |
| CPD- 018 | 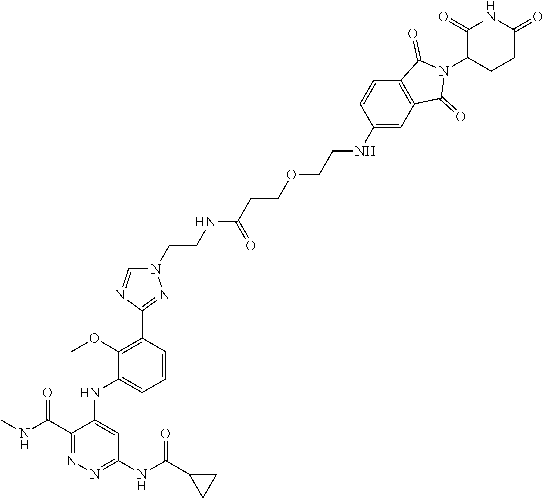 [SMILES: CNC(=O)c1nnc(NC(=O)C2CC2)cc1Nc1cccc(-c2ncn(CCNC(=O)CCOCCNc3ccc4c(c3)C(=O)N(C3CCC(=O)NC3=O)C4=O)n2)c1OC] |
| CPD- 019 | 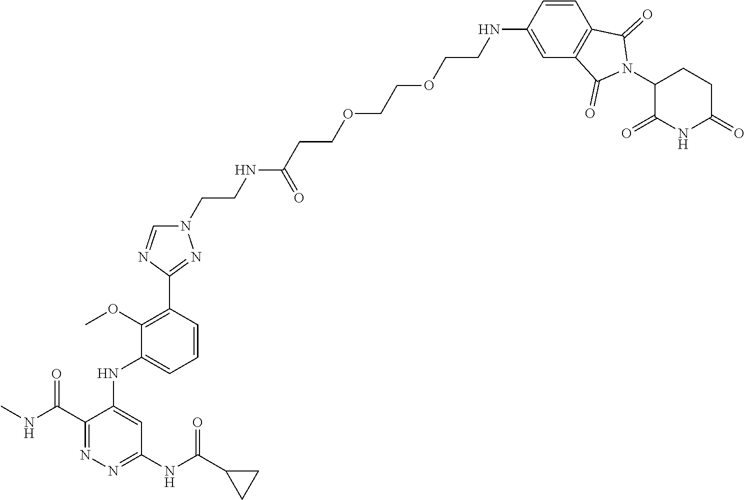 [SMILES: CNC(=O)c1nnc(NC(=O)C2CC2)cc1Nc1cccc(-c2ncn(CCNC(=O)CCOCCOCCNc3ccc4c(c3)C(=O)N(C3CCC(=O)NC3=O)C4=O)n2)c1OC] |
| CPD- 020 | 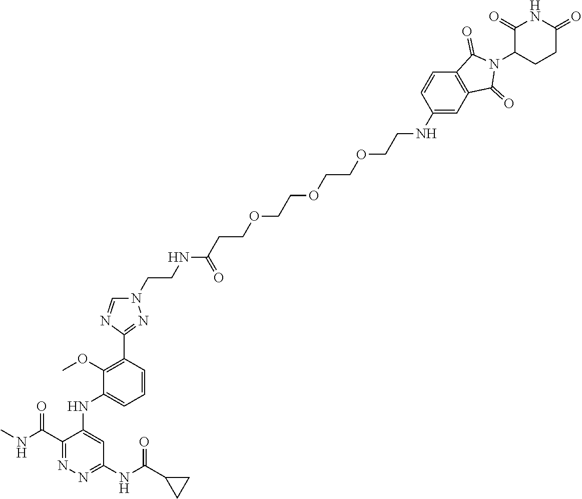 [SMILES: CNC(=O)c1nnc(NC(=O)C2CC2)cc1Nc1cccc(-c2ncn(CCNC(=O)CCOCCOCCOCCNc3ccc4c(c3)C(=O)N(C3CCC(=O)NC3=O)C4=O)n2)c1OC] |
| CPD- 021 | 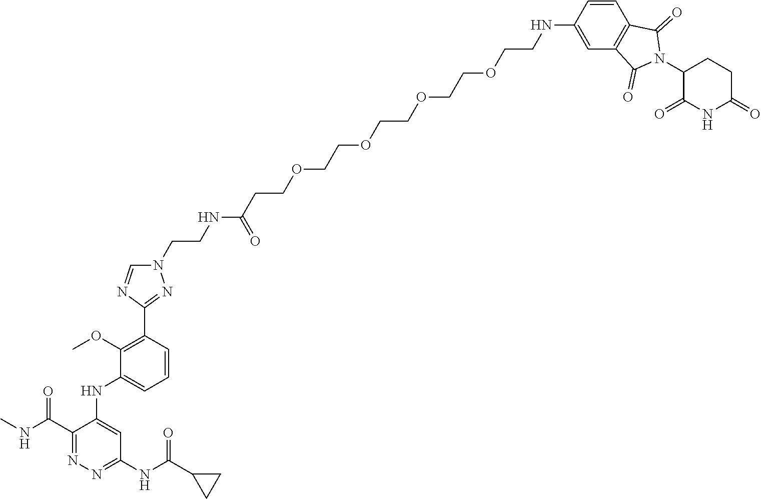 [SMILES: CNC(=O)c1nnc(NC(=O)C2CC2)cc1Nc1cccc(-c2ncn(CCNC(=O)CCOCCOCCOCCOCCNc3ccc4c(c3)C(=O)N(C3CCC(=O)NC3=O)C4=O)n2)c1OC] |
| CPD- 022 | 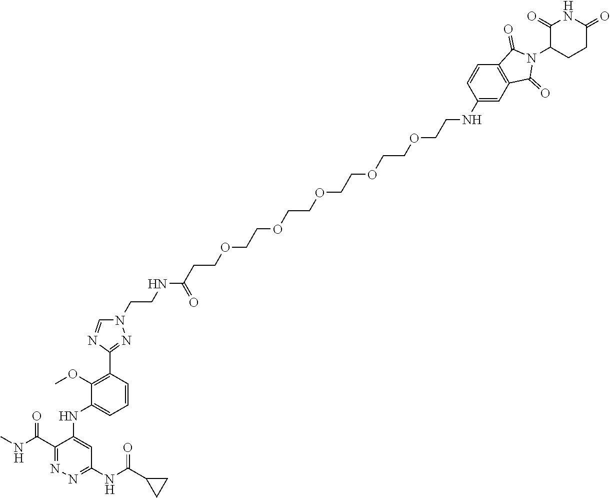 [SMILES: CNC(=O)c1nnc(NC(=O)C2CC2)cc1Nc1cccc(-c2ncn(CCNC(=O)CCOCCOCCOCCOCCOCCNc3ccc4c(c3)C(=O)N(C3CCC(=O)NC3=O)C4=O)n2)c1OC] |
| CPD- 023 | 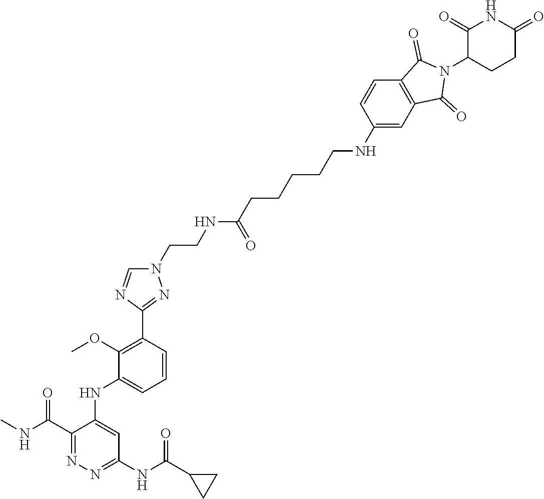 [SMILES: CNC(=O)c1nnc(NC(=O)C2CC2)cc1Nc1cccc(-c2ncn(CCNC(=O)CCCCCNc3ccc4c(c3)C(=O)N(C3CCC(=O)NC3=O)C4=O)n2)c1OC] |
| CPD- 024 | 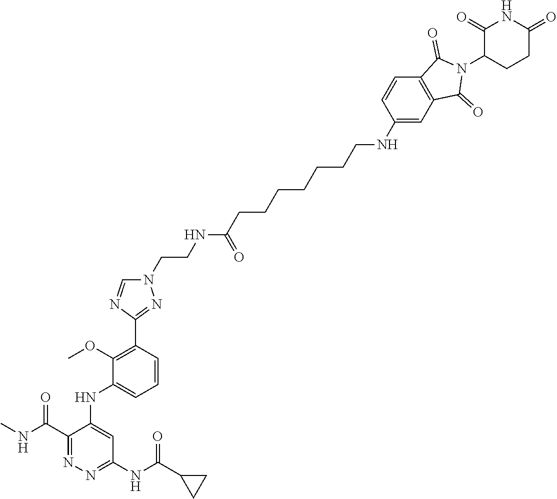 [SMILES: CNC(=O)c1nnc(NC(=O)C2CC2)cc1Nc1cccc(-c2ncn(CCNC(=O)CCCCCCCNc3ccc4c(c3)C(=O)N(C3CCC(=O)NC3=O)C4=O)n2)c1OC] |
| CPD- 025 | 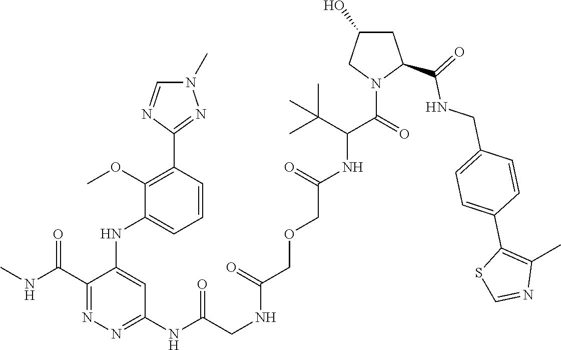 |
| CPD- 026 | 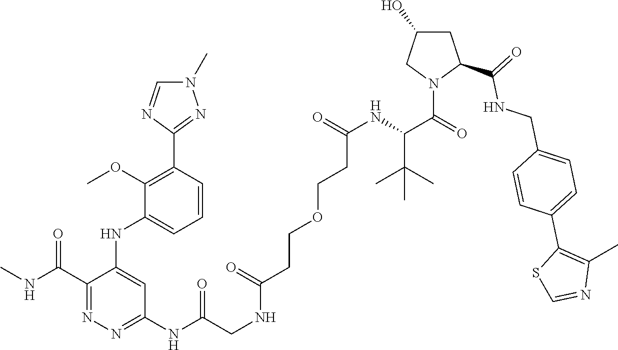 |
| CPD -027 | 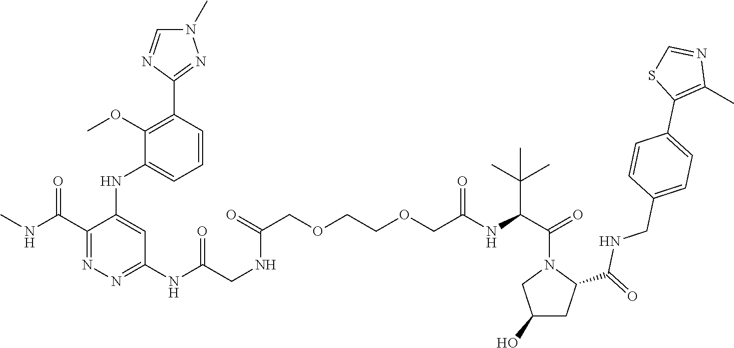 |
| CPD- 028 | 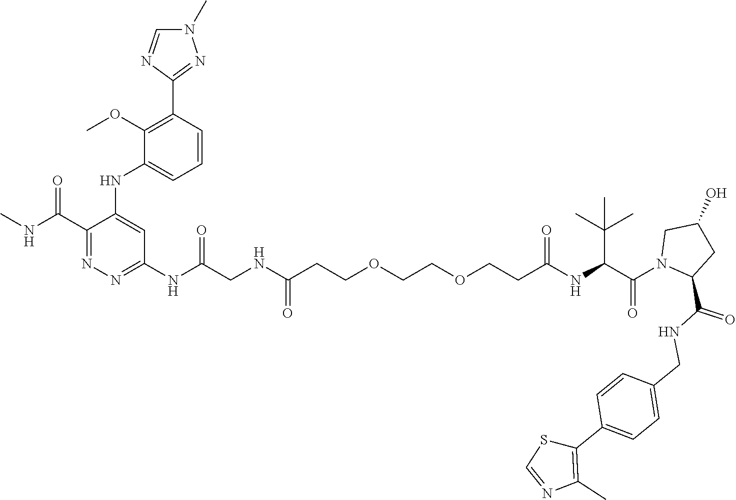 [SMILES: CNC(=O)c1nnc(NC(=O)CNC(=O)CCOCCOCCC(=O)N[C@H](C(=O)N2C[C@H](O)C[C@H]2C(=O)NCc2ccc(-c3scnc3C)cc2)C(C)(C)C)cc1Nc1cccc(-c2ncn(C)n2)c1OC] |
| CPD- 029 | 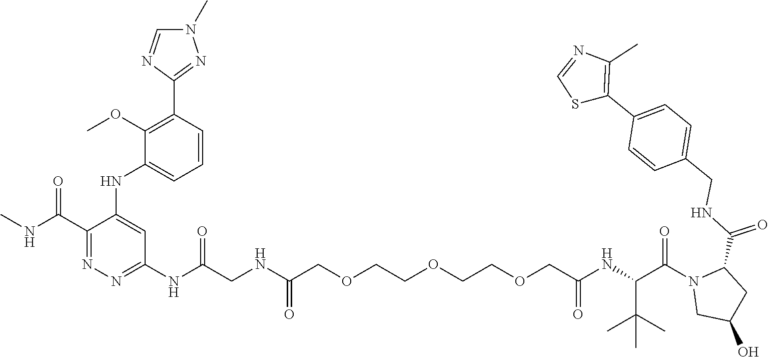 [SMILES: CNC(=O)c1nnc(NC(=O)CNC(=O)COCCOCCOCC(=O)N[C@H](C(=O)N2C[C@H](O)C[C@H]2C(=O)NCc2ccc(-c3scnc3C)cc2)C(C)(C)C)cc1Nc1cccc(-c2ncn(C)n2)c1OC] |
| CPD- 030 | 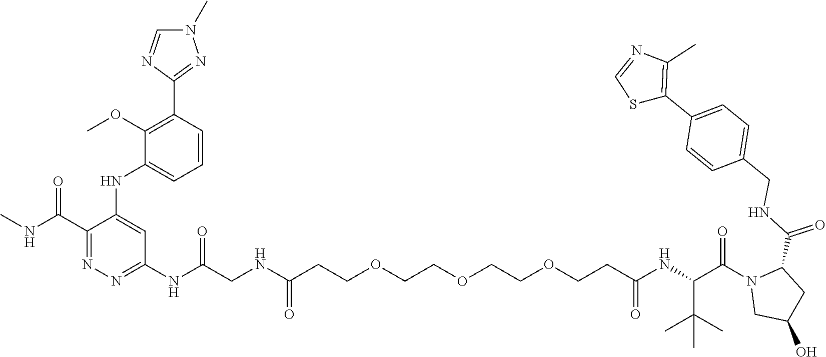 [SMILES: CNC(=O)c1nnc(NC(=O)CNC(=O)CCOCCOCCOCCC(=O)N[C@H](C(=O)N2C[C@H](O)C[C@H]2C(=O)NCc2ccc(-c3scnc3C)cc2)C(C)(C)C)cc1Nc1cccc(-c2ncn(C)n2)c1OC] |
| CPD- 031 | 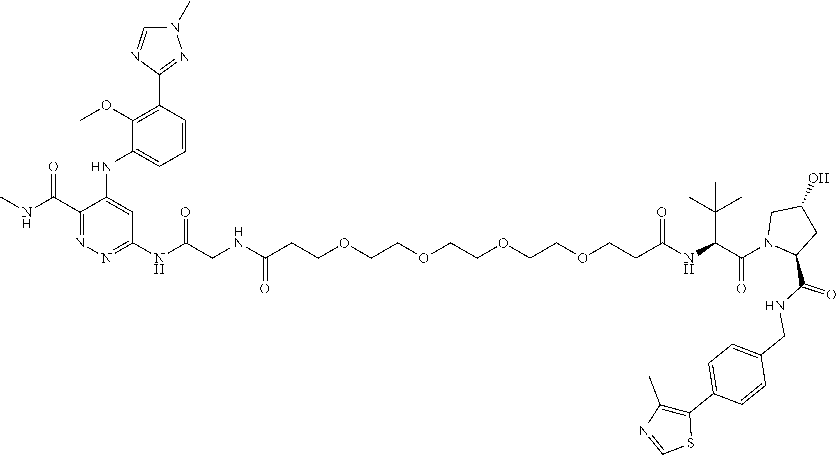 [SMILES: CNC(=O)c1nnc(NC(=O)CNC(=O)CCOCCOCCOCCOCCC(=O)N[C@H](C(=O)N2C[C@H](O)C[C@H]2C(=O)NCc2ccc(-c3scnc3C)cc2)C(C)(C)C)cc1Nc1cccc(-c2ncn(C)n2)c1OC] |
| CPD- 032 | 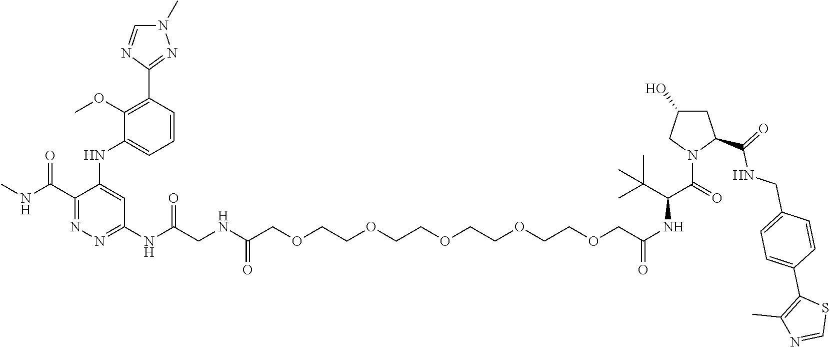 [SMILES: CNC(=O)c1nnc(NC(=O)CNC(=O)COCCOCCOCCOCCOCC(=O)N[C@H](C(=O)N2C[C@H](O)C[C@H]2C(=O)NCc2ccc(-c3scnc3C)cc2)C(C)(C)C)cc1Nc1cccc(-c2ncn(C)n2)c1OC] |
| CPD- 033 | 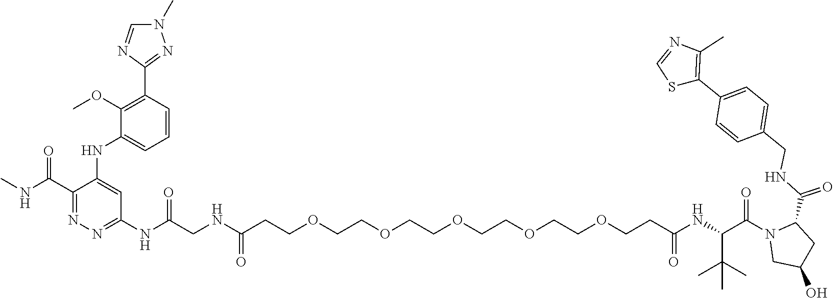 [SMILES: CNC(=O)c1nnc(NC(=O)CNC(=O)CCOCCOCCOCCOCCOCCC(=O)N[C@H](C(=O)N2C[C@H](O)C[C@H]2C(=O)NCc2ccc(-c3scnc3C)cc2)C(C)(C)C)cc1Nc1cccc(-c2ncn(C)n2)c1OC] |
| CPD- 034 | 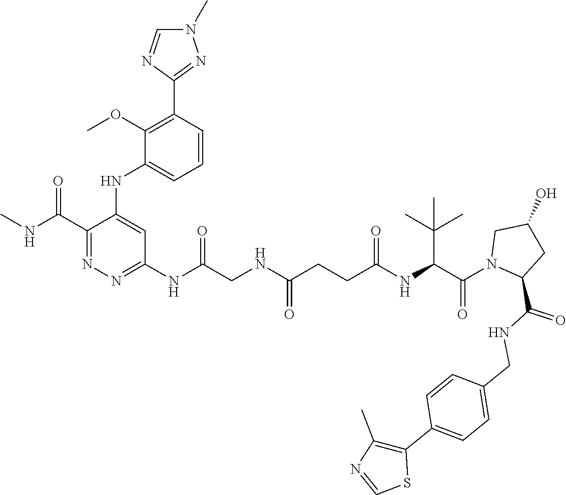 [SMILES: CNC(=O)c1nnc(NC(=O)CNC(=O)CCC(=O)N[C@H](C(=O)N2C[C@H](O)C[C@H]2C(=O)NCc2ccc(-c3scnc3C)cc2)C(C)(C)C)cc1Nc1cccc(-c2ncn(C)n2)c1OC] |
| CPD- 035 | 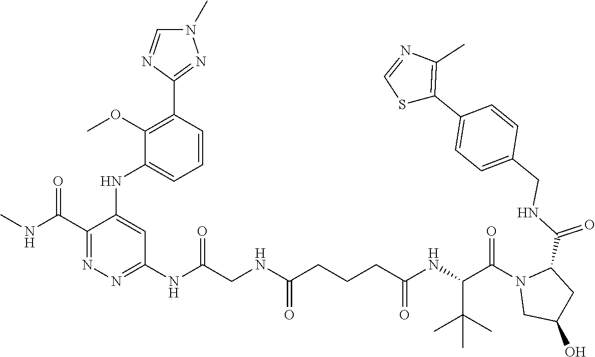 [SMILES: CNC(=O)c1nnc(NC(=O)CNC(=O)CCCC(=O)N[C@H](C(=O)N2C[C@H](O)C[C@H]2C(=O)NCc2ccc(-c3scnc3C)cc2)C(C)(C)C)cc1Nc1cccc(-c2ncn(C)n2)c1OC] |
| CPD- 036 | 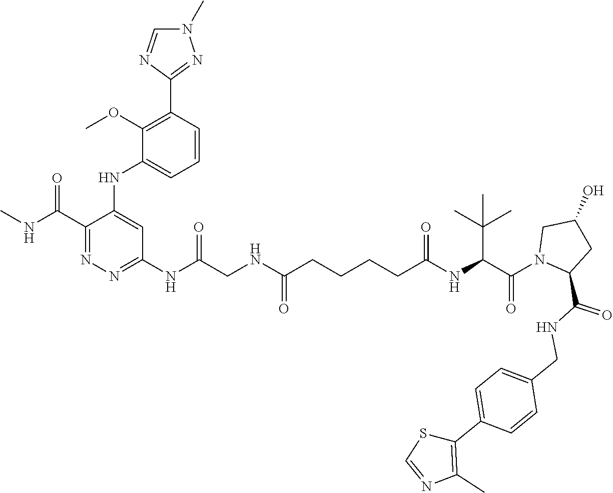 [SMILES: CNC(=O)c1nnc(NC(=O)CNC(=O)CCCCC(=O)N[C@H](C(=O)N2C[C@H](O)C[C@H]2C(=O)NCc2ccc(-c3scnc3C)cc2)C(C)(C)C)cc1Nc1cccc(-c2ncn(C)n2)c1OC] |
| CPD- 037 | 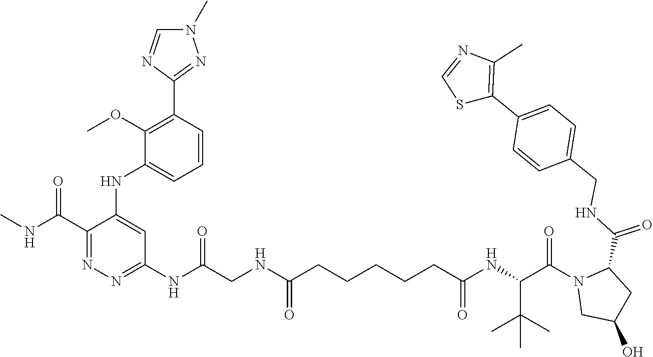 [SMILES: CNC(=O)c1nnc(NC(=O)CNC(=O)CCCCCC(=O)N[C@H](C(=O)N2C[C@H](O)C[C@H]2C(=O)NCc2ccc(-c3scnc3C)cc2)C(C)(C)C)cc1Nc1cccc(-c2ncn(C)n2)c1OC] |
| CPD- 038 | 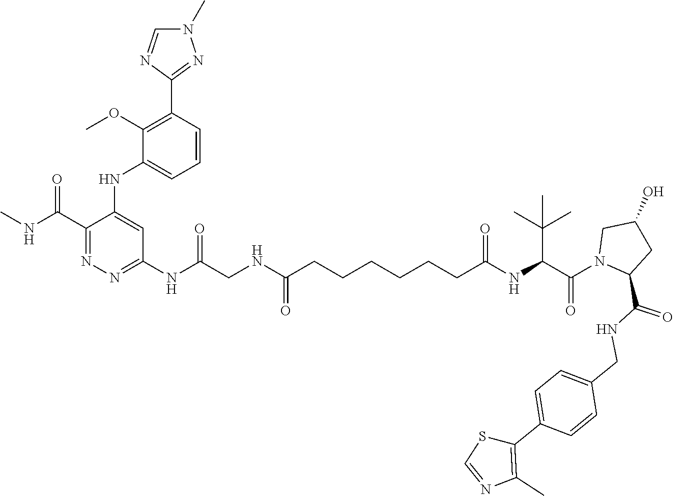 [SMILES: CNC(=O)c1nnc(NC(=O)CNC(=O)CCCCCCC(=O)N[C@H](C(=O)N2C[C@H](O)C[C@H]2C(=O)NCc2ccc(-c3scnc3C)cc2)C(C)(C)C)cc1Nc1cccc(-c2ncn(C)n2)c1OC] |
| CPD- 039 | 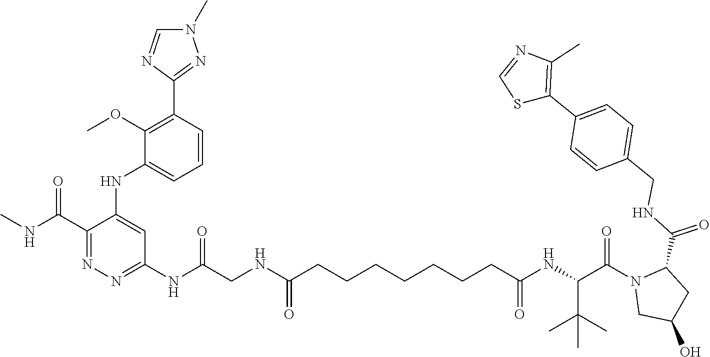 [SMILES: CNC(=O)c1nnc(NC(=O)CNC(=O)CCCCCCCC(=O)N[C@H](C(=O)N2C[C@H](O)C[C@H]2C(=O)NCc2ccc(-c3scnc3C)cc2)C(C)(C)C)cc1Nc1cccc(-c2ncn(C)n2)c1OC] |
| CPD- 040 | 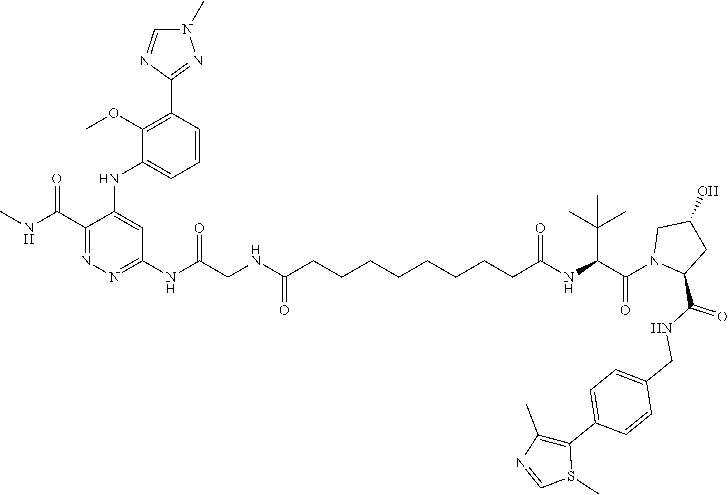 |
| CPD- 041 | 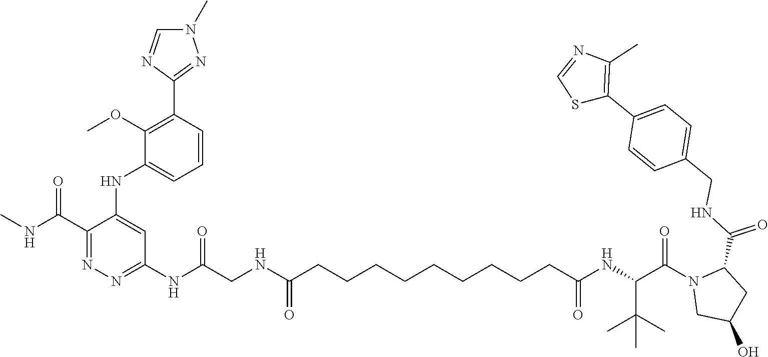 [SMILES: CNC(=O)c1nnc(NC(=O)CNC(=O)CCCCCCCCCC(=O)N[C@H](C(=O)N2C[C@H](O)C[C@H]2C(=O)NCc2ccc(-c3scnc3C)cc2)C(C)(C)C)cc1Nc1cccc(-c2ncn(C)n2)c1OC] |
| CPD- 042 | 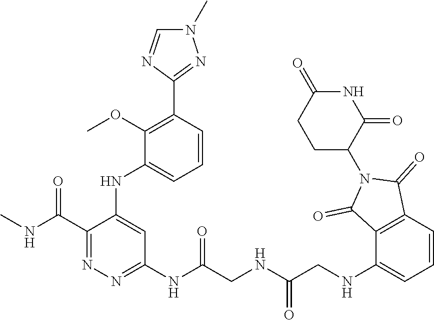 [SMILES: CNC(=O)c1nnc(NC(=O)CNC(=O)CNc2cccc3c2C(=O)N(C2CCC(=O)NC2=O)C3=O)cc1Nc1cccc(-c2ncn(C)n2)c1OC] |
| CPD- 043 | 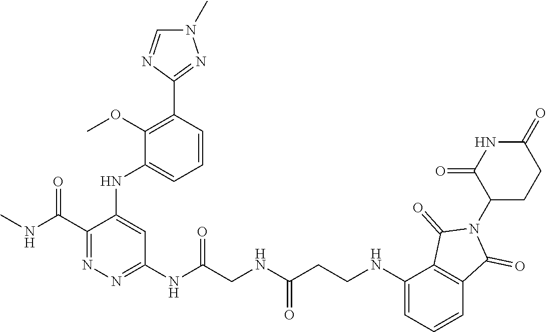 [SMILES: CNC(=O)c1nnc(NC(=O)CNC(=O)CCNc2cccc3c2C(=O)N(C2CCC(=O)NC2=O)C3=O)cc1Nc1cccc(-c2ncn(C)n2)c1OC] |
| CPD- 044 | 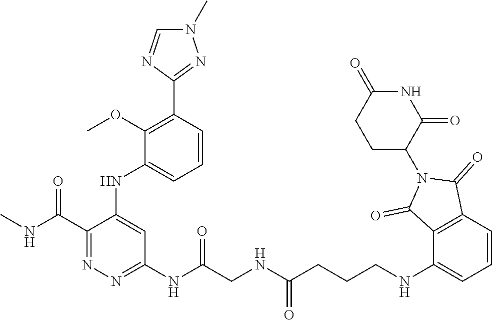 [SMILES: CNC(=O)c1nnc(NC(=O)CNC(=O)CCCNc2cccc3c2C(=O)N(C2CCC(=O)NC2=O)C3=O)cc1Nc1cccc(-c2ncn(C)n2)c1OC] |
| CPD- 045 | 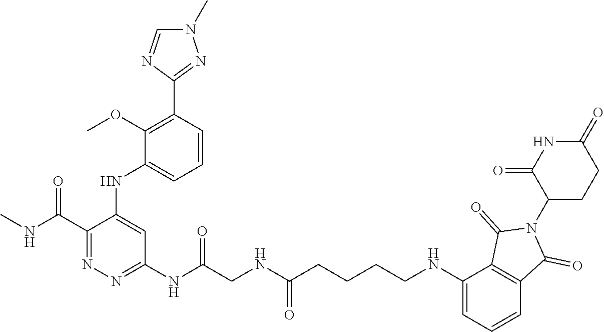 [SMILES: CNC(=O)c1nnc(NC(=O)CNC(=O)CCCCNc2cccc3c2C(=O)N(C2CCC(=O)NC2=O)C3=O)cc1Nc1cccc(-c2ncn(C)n2)c1OC] |
| CPD- 046 | 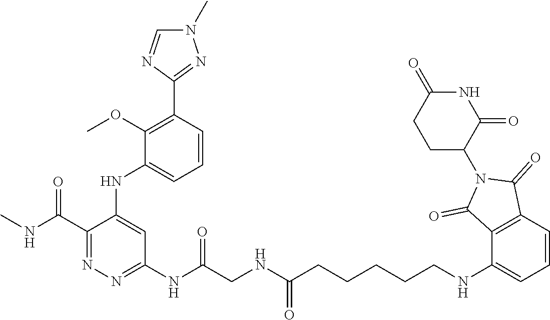 [SMILES: CNC(=O)c1nnc(NC(=O)CNC(=O)CCCCCNc2cccc3c2C(=O)N(C2CCC(=O)NC2=O)C3=O)cc1Nc1cccc(-c2ncn(C)n2)c1OC] |
| CPD- 047 | 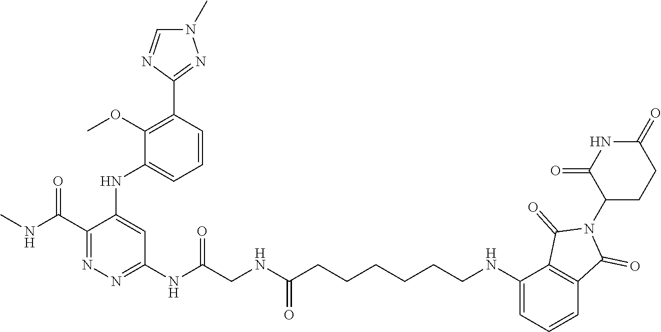 [SMILES: CNC(=O)c1nnc(NC(=O)CNC(=O)CCCCCCNc2cccc3c2C(=O)N(C2CCC(=O)NC2=O)C3=O)cc1Nc1cccc(-c2ncn(C)n2)c1OC] |
| CPD- 048 | 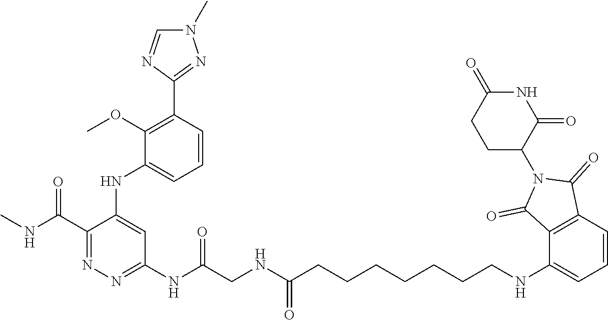 [SMILES: CNC(=O)c1nnc(NC(=O)CNC(=O)CCCCCCCNc2cccc3c2C(=O)N(C2CCC(=O)NC2=O)C3=O)cc1Nc1cccc(-c2ncn(C)n2)c1OC] |
| CPD- 049 | 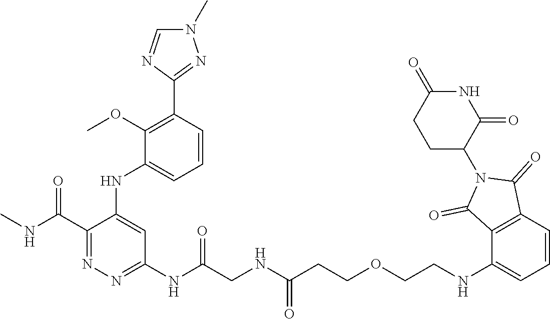 [SMILES: CNC(=O)c1nnc(NC(=O)CNC(=O)CCOCCNc2cccc3c2C(=O)N(C2CCC(=O)NC2=O)C3=O)cc1Nc1cccc(-c2ncn(C)n2)c1OC] |
| CPD- 050 | 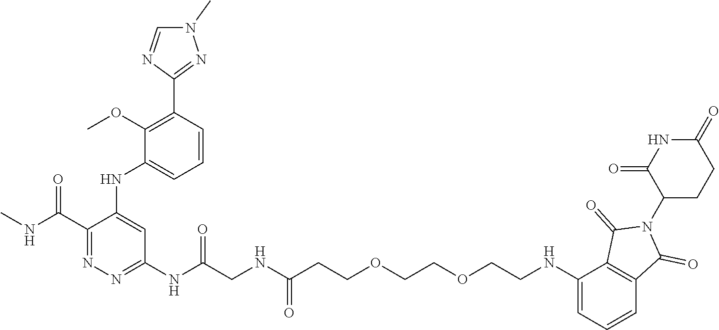 [SMILES: CNC(=O)c1nnc(NC(=O)CNC(=O)CCOCCOCCNc2cccc3c2C(=O)N(C2CCC(=O)NC2=O)C3=O)cc1Nc1cccc(-c2ncn(C)n2)c1OC] |
| CPD- 051 | 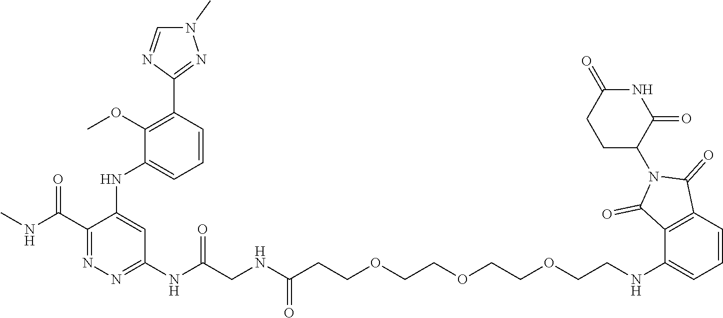 [SMILES: CNC(=O)c1nnc(NC(=O)CNC(=O)CCOCCOCCOCCNc2cccc3c2C(=O)N(C2CCC(=O)NC2=O)C3=O)cc1Nc1cccc(-c2ncn(C)n2)c1OC] |
| CPD- 052 | 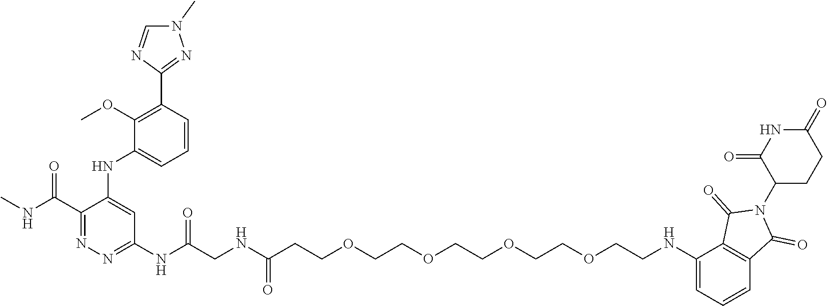 [SMILES: CNC(=O)c1nnc(NC(=O)CNC(=O)CCOCCOCCOCCOCCNc2cccc3c2C(=O)N(C2CCC(=O)NC2=O)C3=O)cc1Nc1cccc(-c2ncn(C)n2)c1OC] |
| CPD- 053 | 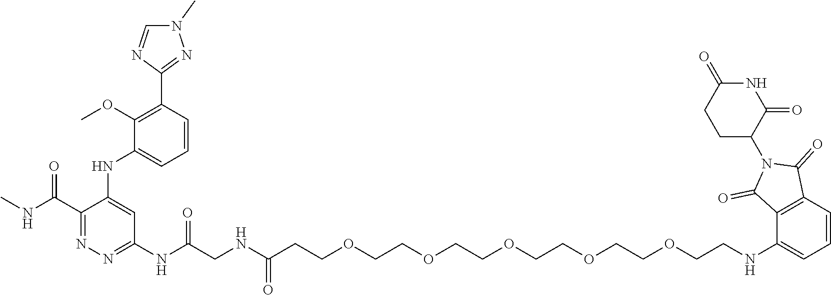 [SMILES: CNC(=O)c1nnc(NC(=O)CNC(=O)CCOCCOCCOCCOCCOCCNc2cccc3c2C(=O)N(C2CCC(=O)NC2=O)C3=O)cc1Nc1cccc(-c2ncn(C)n2)c1OC] |
| CPD- 054 | 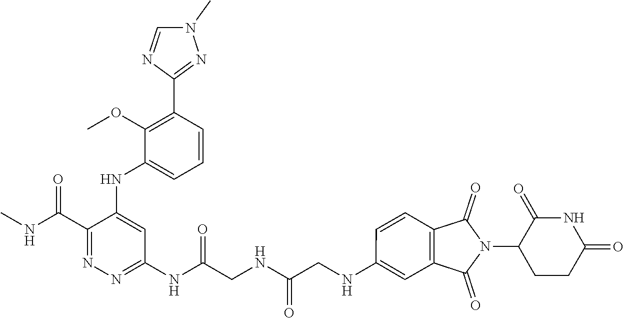 [SMILES: CNC(=O)c1nnc(NC(=O)CNC(=O)CNc2ccc3c(c2)C(=O)N(C2CCC(=O)NC2=O)C3=O)cc1Nc1cccc(-c2ncn(C)n2)c1OC] |
| CPD- 055 | 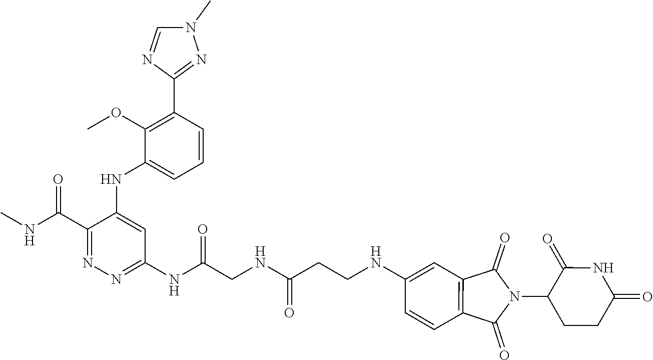 [SMILES: CNC(=O)c1nnc(NC(=O)CNC(=O)CCNc2ccc3c(c2)C(=O)N(C2CCC(=O)NC2=O)C3=O)cc1Nc1cccc(-c2ncn(C)n2)c1OC] |
| CPD- 056 | 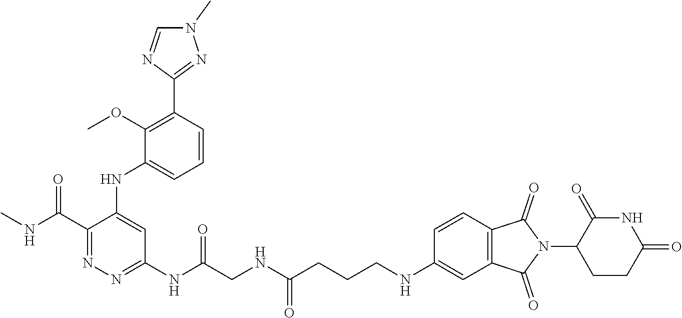 [SMILES: CNC(=O)c1nnc(NC(=O)CNC(=O)CCCNc2ccc3c(c2)C(=O)N(C2CCC(=O)NC2=O)C3=O)cc1Nc1cccc(-c2ncn(C)n2)c1OC] |
| CPD- 057 | 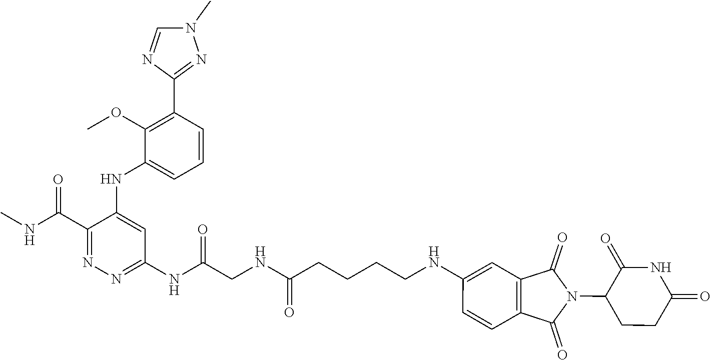 [SMILES: CNC(=O)c1nnc(NC(=O)CNC(=O)CCCCNc2ccc3c(c2)C(=O)N(C2CCC(=O)NC2=O)C3=O)cc1Nc1cccc(-c2ncn(C)n2)c1OC] |
| CPD- 058 | 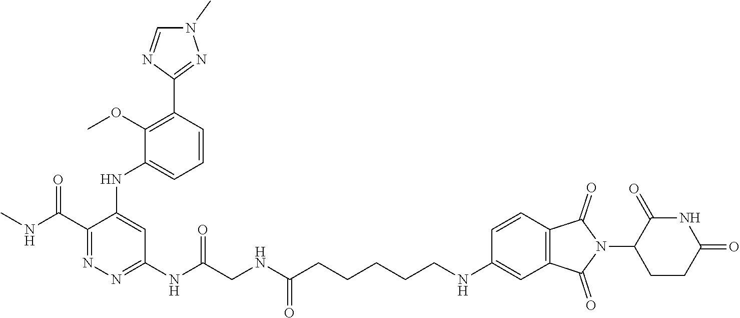 [SMILES: CNC(=O)c1nnc(NC(=O)CNC(=O)CCCCCNc2ccc3c(c2)C(=O)N(C2CCC(=O)NC2=O)C3=O)cc1Nc1cccc(-c2ncn(C)n2)c1OC] |
| CPD- 059 | 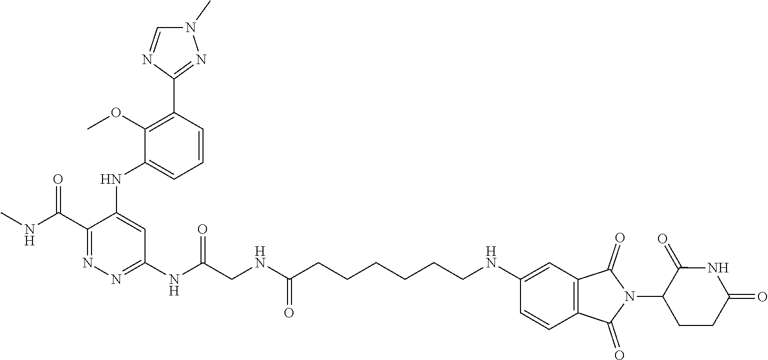 [SMILES: CNC(=O)c1nnc(NC(=O)CNC(=O)CCCCCCNc2ccc3c(c2)C(=O)N(C2CCC(=O)NC2=O)C3=O)cc1Nc1cccc(-c2ncn(C)n2)c1OC] |
| CPD- 060 | 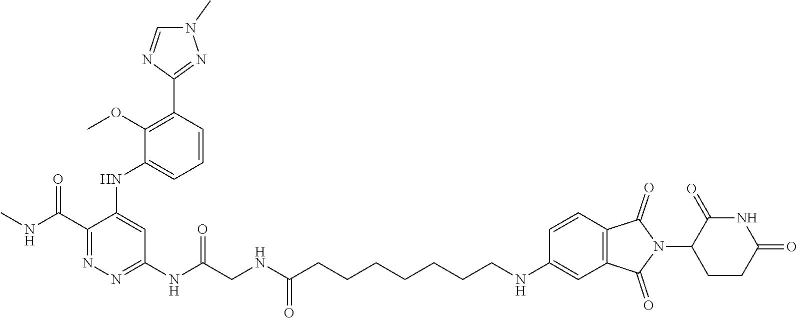 [SMILES: CNC(=O)c1nnc(NC(=O)CNC(=O)CCCCCCCNc2ccc3c(c2)C(=O)N(C2CCC(=O)NC2=O)C3=O)cc1Nc1cccc(-c2ncn(C)n2)c1OC] |
| CPD- 061 | 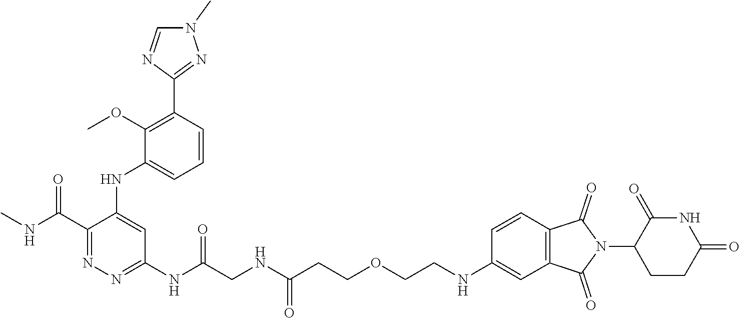 [SMILES: CNC(=O)c1nnc(NC(=O)CNC(=O)CCOCCNc2ccc3c(c2)C(=O)N(C2CCC(=O)NC2=O)C3=O)cc1Nc1cccc(-c2ncn(C)n2)c1OC] |
| CPD- 062 | 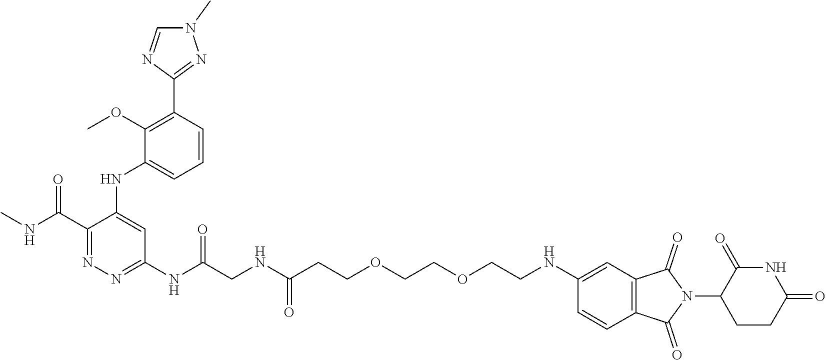 [SMILES: CNC(=O)c1nnc(NC(=O)CNC(=O)CCOCCOCCNc2ccc3c(c2)C(=O)N(C2CCC(=O)NC2=O)C3=O)cc1Nc1cccc(-c2ncn(C)n2)c1OC] |
| CPD- 063 | 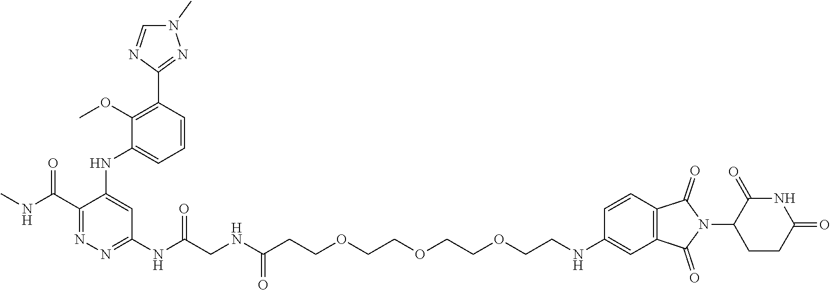 [SMILES: CNC(=O)c1nnc(NC(=O)CNC(=O)CCOCCOCCOCCNc2ccc3c(c2)C(=O)N(C2CCC(=O)NC2=O)C3=O)cc1Nc1cccc(-c2ncn(C)n2)c1OC] |
| CPD- 064 | 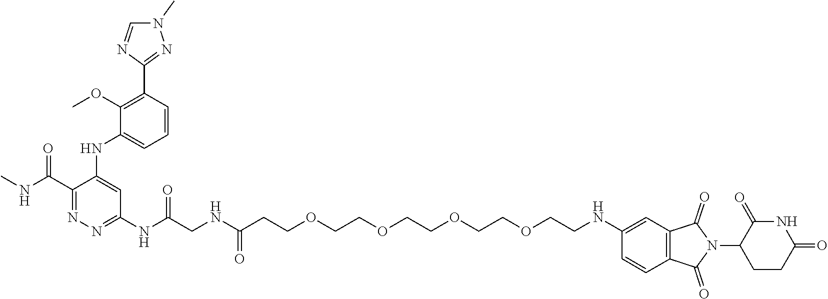 [SMILES: CNC(=O)c1nnc(NC(=O)CNC(=O)CCOCCOCCOCCOCCNc2ccc3c(c2)C(=O)N(C2CCC(=O)NC2=O)C3=O)cc1Nc1cccc(-c2ncn(C)n2)c1OC] |
| CPD- 065 | 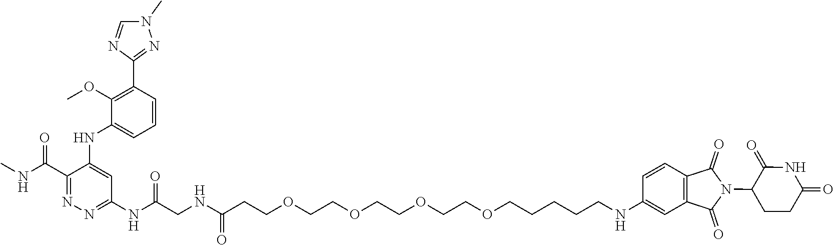 [SMILES: CNC(=O)c1nnc(NC(=O)CNC(=O)CCOCCOCCOCCOCCCCCNc2ccc3c(c2)C(=O)N(C2CCC(=O)NC2=O)C3=O)cc1Nc1cccc(-c2ncn(C)n2)c1OC] |
| CPD- 066 | 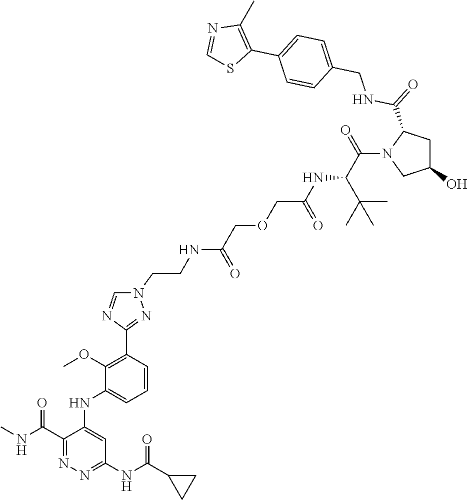 [SMILES: CNC(=O)c1nnc(NC(=O)C2CC2)cc1Nc1cccc(-c2ncn(CCNC(=O)COCC(=O)N[C@H](C(=O)N3C[C@H](O)C[C@H]3C(=O)NCc3ccc(-c4scnc4C)cc3)C(C)(C)C)n2)c1OC] |
| CPD- 067 | 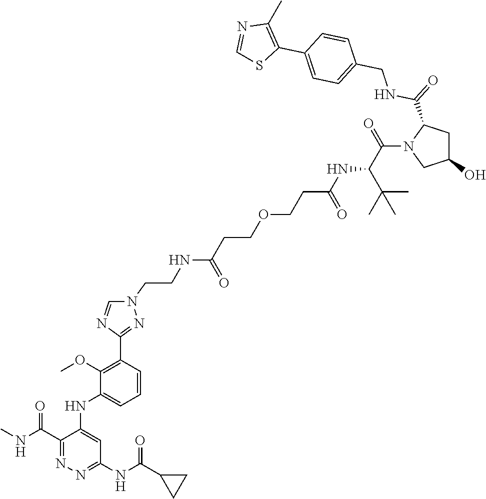 [SMILES: CNC(=O)c1nnc(NC(=O)C2CC2)cc1Nc1cccc(-c2ncn(CCNC(=O)CCOCCC(=O)N[C@H](C(=O)N3C[C@H](O)C[C@H]3C(=O)NCc3ccc(-c4scnc4C)cc3)C(C)(C)C)n2)c1OC] |
| CPD- 068 | 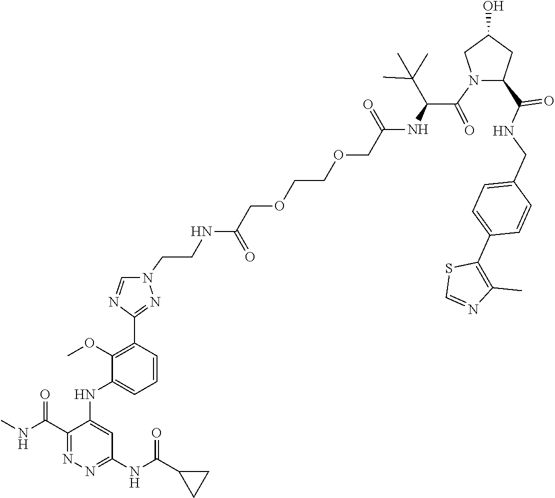 [SMILES: CNC(=O)c1nnc(NC(=O)C2CC2)cc1Nc1cccc(-c2ncn(CCNC(=O)COCCOCC(=O)N[C@H](C(=O)N3C[C@H](O)C[C@H]3C(=O)NCc3ccc(-c4scnc4C)cc3)C(C)(C)C)n2)c1OC] |
| CPD- 069 | 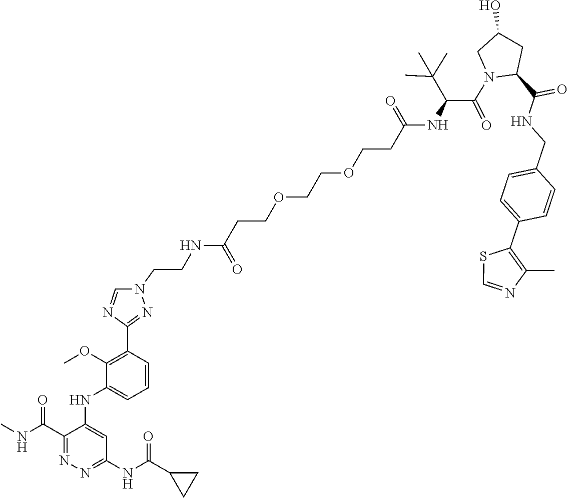 [SMILES: CNC(=O)c1nnc(NC(=O)C2CC2)cc1Nc1cccc(-c2ncn(CCNC(=O)CCOCCOCCC(=O)N[C@H](C(=O)N3C[C@H](O)C[C@H]3C(=O)NCc3ccc(-c4scnc4C)cc3)C(C)(C)C)n2)c1OC] |
| CPD- 070 | 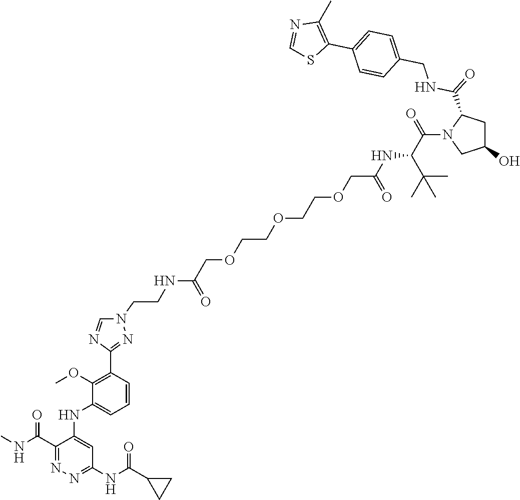 [SMILES: CNC(=O)c1nnc(NC(=O)C2CC2)cc1Nc1cccc(-c2ncn(CCNC(=O)COCCOCCOCC(=O)N[C@H](C(=O)N3C[C@H](O)C[C@H]3C(=O)NCc3ccc(-c4scnc4C)cc3)C(C)(C)C)n2)c1OC] |
| CPD- 071 | 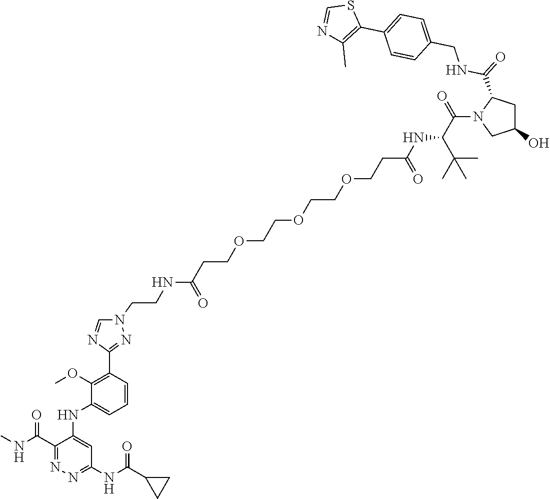 [SMILES: CNC(=O)c1nnc(NC(=O)C2CC2)cc1Nc1cccc(-c2ncn(CCNC(=O)CCOCCOCCOCCC(=O)N[C@H](C(=O)N3C[C@H](O)C[C@H]3C(=O)NCc3ccc(-c4scnc4C)cc3)C(C)(C)C)n2)c1OC] |
| CPD- 072 | 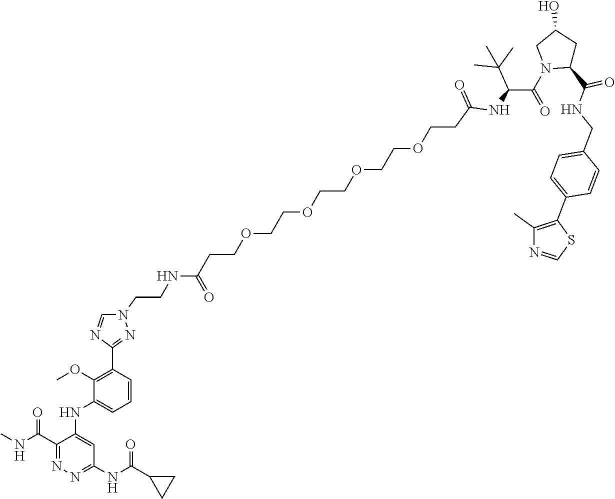 [SMILES: CNC(=O)c1nnc(NC(=O)C2CC2)cc1Nc1cccc(-c2ncn(CCNC(=O)CCOCCOCCOCCOCCC(=O)N[C@H](C(=O)N3C[C@H](O)C[C@H]3C(=O)NCc3ccc(-c4scnc4C)cc3)C(C)(C)C)n2)c1OC] |
| CPD- 073 | 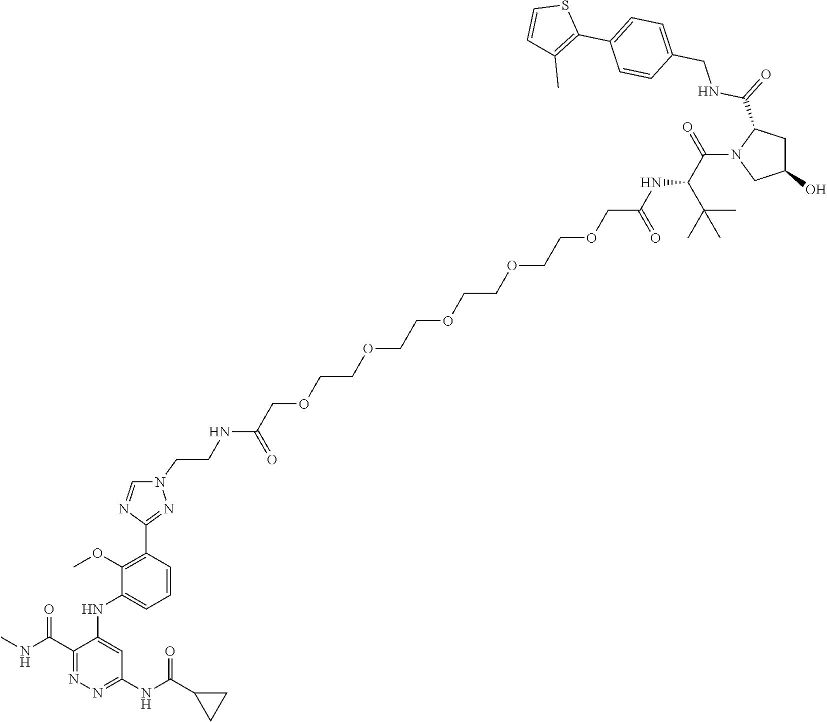 [SMILES: CNC(=O)c1nnc(NC(=O)C2CC2)cc1Nc1cccc(-c2ncn(CCNC(=O)COCCOCCOCCOCCOCC(=O)N[C@H](C(=O)N3C[C@H](O)C[C@H]3C(=O)NCc3ccc(-c4sccc4C)cc3)C(C)(C)C)n2)c1OC] |
| CPD- 074 | 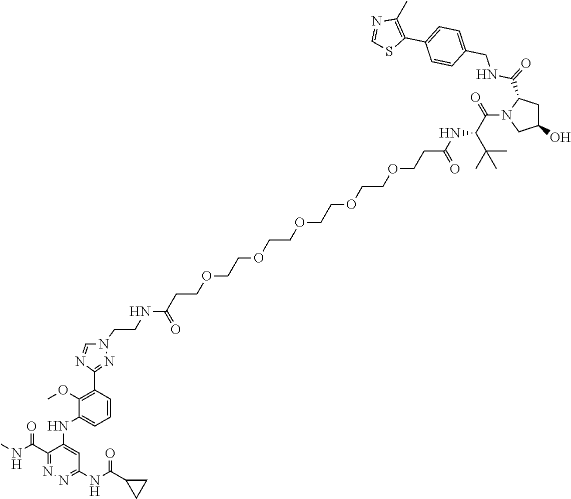 [SMILES: CNC(=O)c1nnc(NC(=O)C2CC2)cc1Nc1cccc(-c2ncn(CCNC(=O)CCOCCOCCOCCOCCOCCC(=O)N[C@H](C(=O)N3C[C@H](O)C[C@H]3C(=O)NCc3ccc(-c4scnc4C)cc3)C(C)(C)C)n2)c1OC] |
| CPD- 075 | 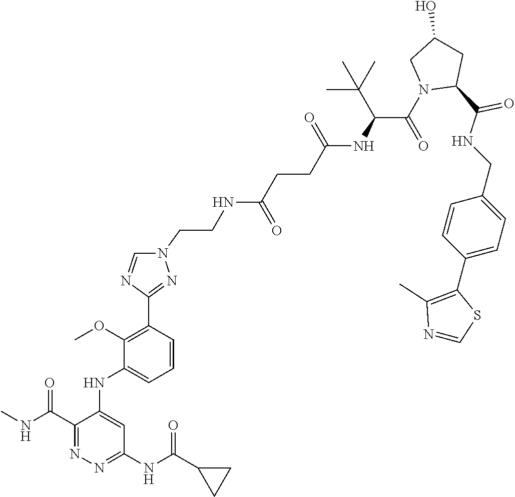 [SMILES: CNC(=O)c1nnc(NC(=O)C2CC2)cc1Nc1cccc(-c2ncn(CCNC(=O)CCC(=O)N[C@H](C(=O)N3C[C@H](O)C[C@H]3C(=O)NCc3ccc(-c4scnc4C)cc3)C(C)(C)C)n2)c1OC] |
| CPD- 076 | 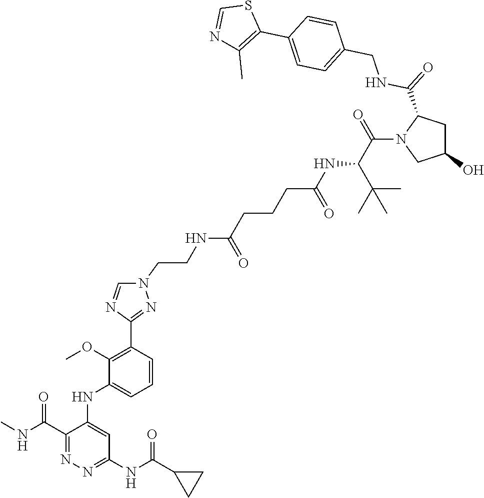 [SMILES: CNC(=O)c1nnc(NC(=O)C2CC2)cc1Nc1cccc(-c2ncn(CCNC(=O)CCCC(=O)N[C@H](C(=O)N3C[C@H](O)C[C@H]3C(=O)NCc3ccc(-c4scnc4C)cc3)C(C)(C)C)n2)c1OC] |
| CPD- 077 | 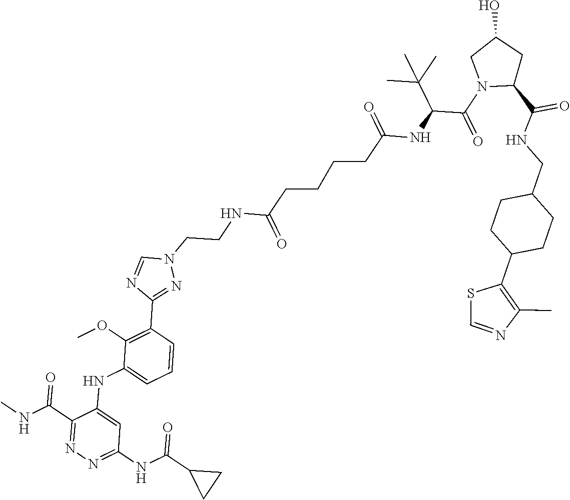 [SMILES: CNC(=O)c1nnc(NC(=O)C2CC2)cc1Nc1cccc(-c2ncn(CCNC(=O)CCCCC(=O)N[C@H](C(=O)N3C[C@H](O)C[C@H]3C(=O)NCC3CCC(c4scnc4C)CC3)C(C)(C)C)n2)c1OC] |
| CPD- 078 | 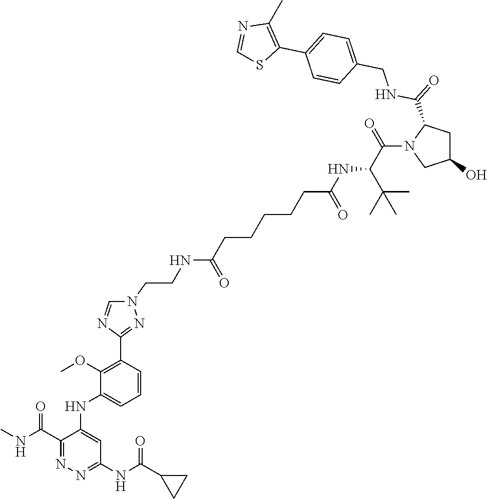 [SMILES: CNC(=O)c1nnc(NC(=O)C2CC2)cc1Nc1cccc(-c2ncn(CCNC(=O)CCCCCC(=O)N[C@H](C(=O)N3C[C@H](O)C[C@H]3C(=O)NCc3ccc(-c4scnc4C)cc3)C(C)(C)C)n2)c1OC] |
| CPD- 079 | 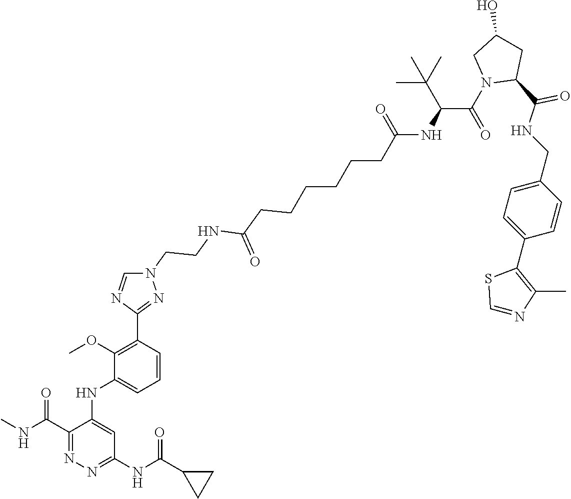 |
| CPD- 080 | 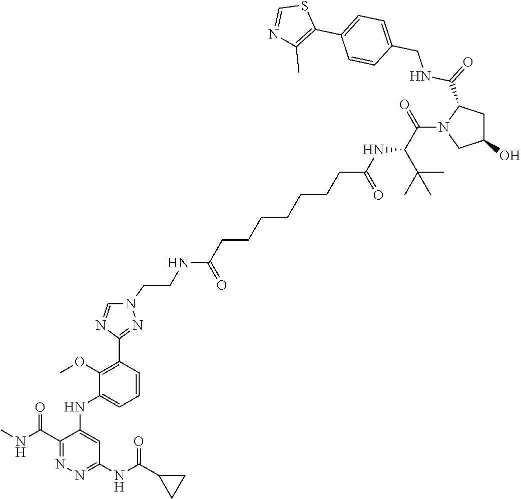 [SMILES: CNC(=O)c1nnc(NC(=O)C2CC2)cc1Nc1cccc(-c2ncn(CCNC(=O)CCCCCCCC(=O)N[C@H](C(=O)N3C[C@H](O)C[C@H]3C(=O)NCc3ccc(-c4scnc4C)cc3)C(C)(C)C)n2)c1OC] |
| CPD- 081 | 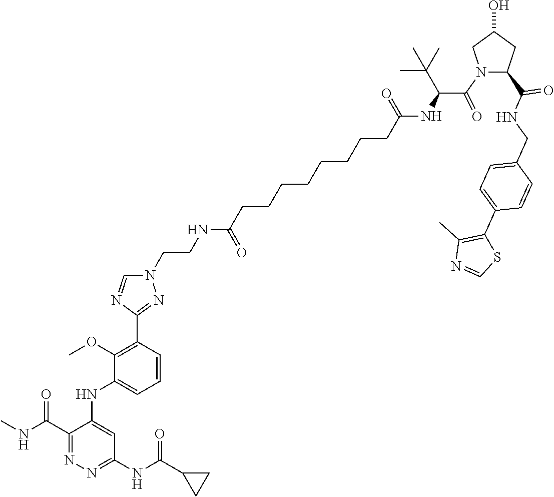 [SMILES: CNC(=O)c1nnc(NC(=O)C2CC2)cc1Nc1cccc(-c2ncn(CCNC(=O)CCCCCCCCC(=O)N[C@H](C(=O)N3C[C@H](O)C[C@H]3C(=O)NCc3ccc(-c4scnc4C)cc3)C(C)(C)C)n2)c1OC] |
| CPD- 082 | 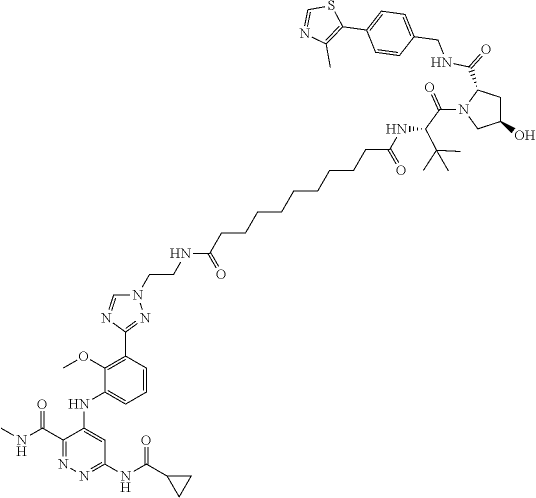 [SMILES: CNC(=O)c1nnc(NC(=O)C2CC2)cc1Nc1cccc(-c2ncn(CCNC(=O)CCCCCCCCCC(=O)N[C@H](C(=O)N3C[C@H](O)C[C@H]3C(=O)NCc3ccc(-c4scnc4C)cc3)C(C)(C)C)n2)c1OC] |
| CPD- 083 | 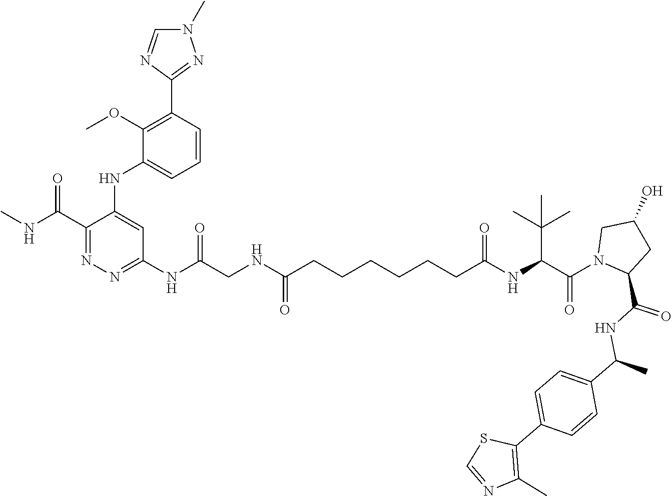 [SMILES: CNC(=O)c1nnc(NC(=O)CNC(=O)CCCCCCC(=O)N[C@H](C(=O)N2C[C@H](O)C[C@H]2C(=O)N[C@@H](C)c2ccc(-c3scnc3C)cc2)C(C)(C)C)cc1Nc1cccc(-c2ncn(C)n2)c1OC] |
| CPD- 084 | 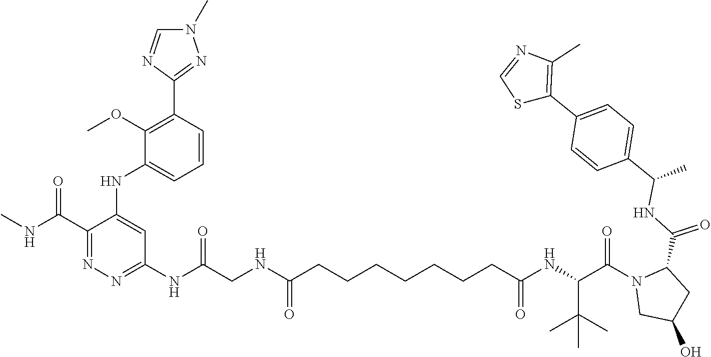 [SMILES: CNC(=O)c1nnc(NC(=O)CNC(=O)CCCCCCCC(=O)N[C@H](C(=O)N2C[C@H](O)C[C@H]2C(=O)N[C@@H](C)c2ccc(-c3scnc3C)cc2)C(C)(C)C)cc1Nc1cccc(-c2ncn(C)n2)c1OC] |
| CPD- 085 | 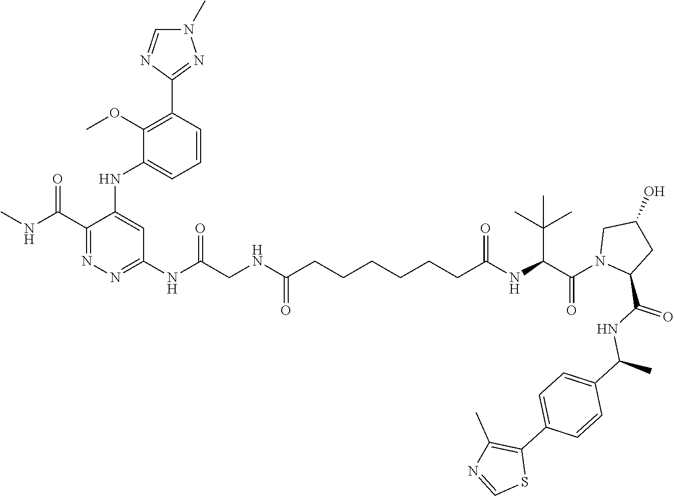 [SMILES: CNC(=O)c1nnc(NC(=O)CNC(=O)CCCCCCC(=O)N[C@H](C(=O)N2C[C@H](O)C[C@H]2C(=O)N[C@@H](C)c2ccc(-c3scnc3C)cc2)C(C)(C)C)cc1Nc1cccc(-c2ncn(C)n2)c1OC] |
| CPD- 086 | 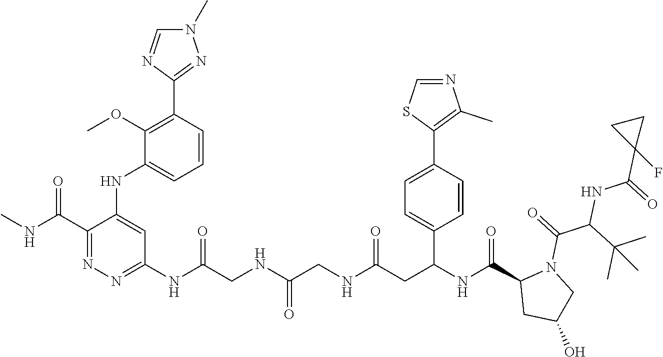 [SMILES: CNC(=O)c1nnc(NC(=O)CNC(=O)CNC(=O)CC(NC(=O)[C@@H]2C[C@@H](O)CN2C(=O)C(NC(=O)C2(F)CC2)C(C)(C)C)c2ccc(-c3scnc3C)cc2)cc1Nc1cccc(-c2ncn(C)n2)c1OC] |
| CPD- 087 | 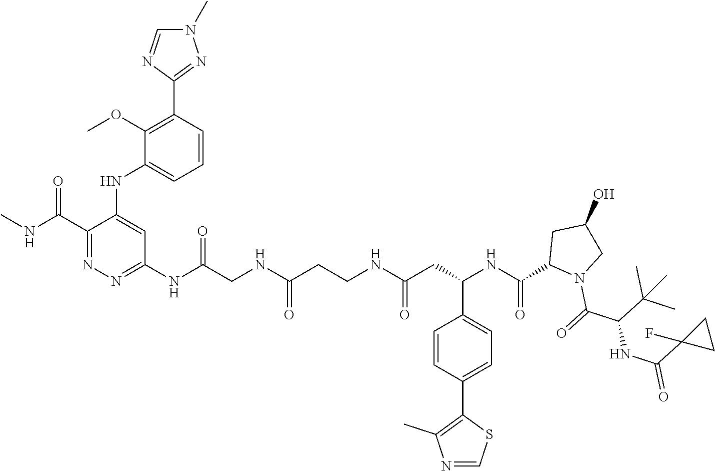 |
| CPD- 088 | 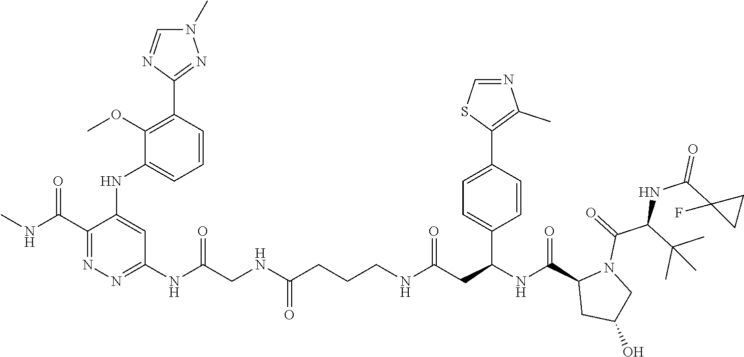 [SMILES: CNC(=O)c1nnc(NC(=O)CNC(=O)CCCNC(=O)C[C@H](NC(=O)[C@@H]2C[C@@H](O)CN2C(=O)[C@@H](NC(=O)C2(F)CC2)C(C)(C)C)c2ccc(-c3scnc3C)cc2)cc1Nc1cccc(-c2ncn(C)n2)c1OC] |
| CPD- 089 | 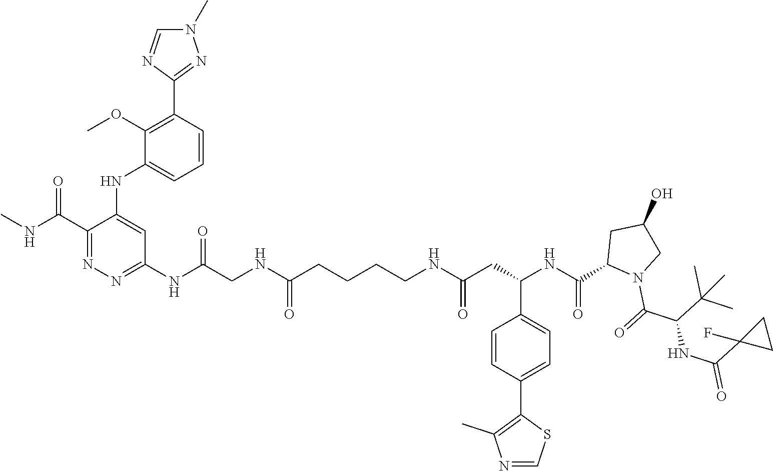 [SMILES: CNC(=O)c1nnc(NC(=O)CNC(=O)CCCCNC(=O)C[C@H](NC(=O)[C@@H]2C[C@@H](O)CN2C(=O)[C@@H](NC(=O)C2(F)CC2)C(C)(C)C)c2ccc(-c3scnc3C)cc2)cc1Nc1cccc(-c2ncn(C)n2)c1OC] |
| CPD- 090 | 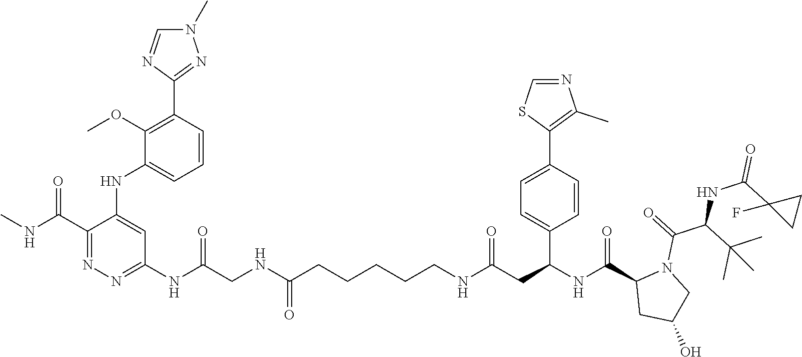 [SMILES: CNC(=O)c1nnc(NC(=O)CNC(=O)CCCCCNC(=O)C[C@H](NC(=O)[C@@H]2C[C@@H](O)CN2C(=O)[C@@H](NC(=O)C2(F)CC2)C(C)(C)C)c2ccc(-c3scnc3C)cc2)cc1Nc1cccc(-c2ncn(C)n2)c1OC] |
| CPD- 091 | 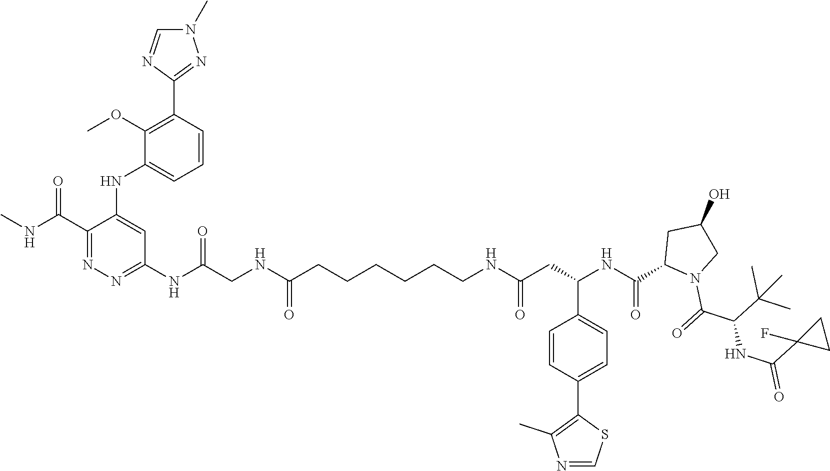 [SMILES: CNC(=O)c1nnc(NC(=O)CNC(=O)CCCCCCNC(=O)C[C@H](NC(=O)[C@@H]2C[C@@H](O)CN2C(=O)[C@@H](NC(=O)C2(F)CC2)C(C)(C)C)c2ccc(-c3scnc3C)cc2)cc1Nc1cccc(-c2ncn(C)n2)c1OC] |
| CPD -092 | 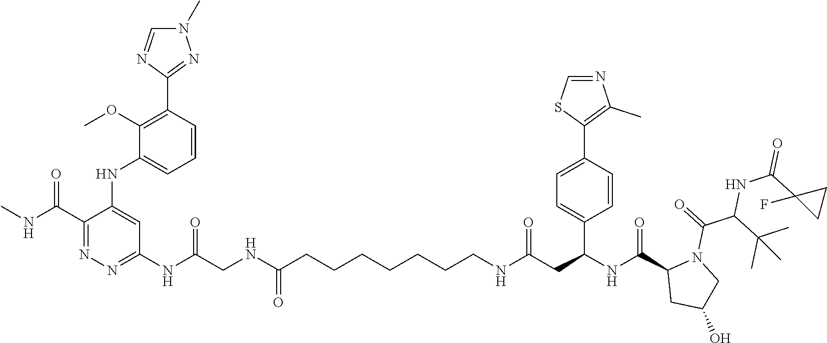 [SMILES: CNC(=O)c1nnc(NC(=O)CNC(=O)CCCCCCCNC(=O)C[C@H](NC(=O)[C@@H]2C[C@@H](O)CN2C(=O)C(NC(=O)C2(F)CC2)C(C)(C)C)c2ccc(-c3scnc3C)cc2)cc1Nc1cccc(-c2ncn(C)n2)c1OC] |
| CPD- 093 | 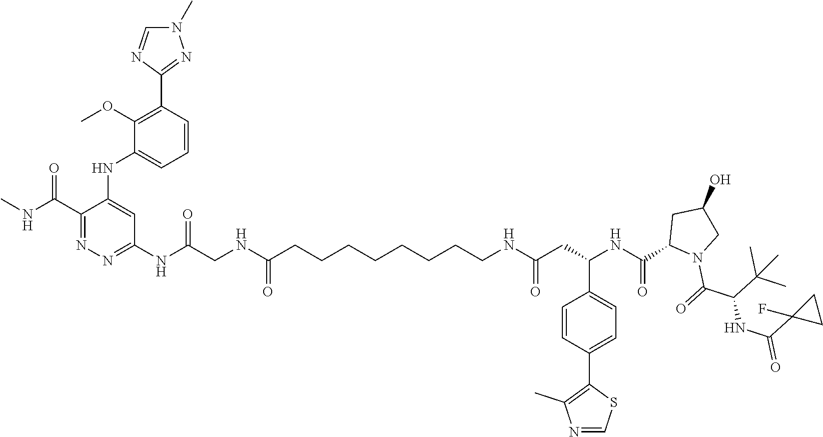 [SMILES: CNC(=O)c1nnc(NC(=O)CNC(=O)CCCCCCCCNC(=O)C[C@H](NC(=O)[C@@H]2C[C@@H](O)CN2C(=O)[C@@H](NC(=O)C2(F)CC2)C(C)(C)C)c2ccc(-c3scnc3C)cc2)cc1Nc1cccc(-c2ncn(C)n2)c1OC] |
| CPD- 094 | 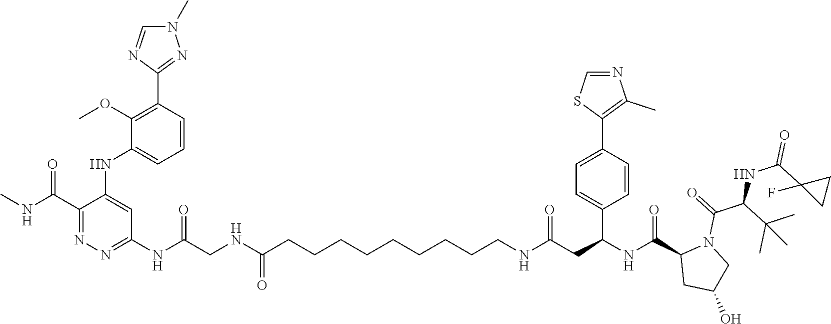 [SMILES: CNC(=O)c1nnc(NC(=O)CNC(=O)CCCCCCCCCNC(=O)C[C@H](NC(=O)[C@@H]2C[C@@H](O)CN2C(=O)[C@@H](NC(=O)C2(F)CC2)C(C)(C)C)c2ccc(-c3scnc3C)cc2)cc1Nc1cccc(-c2ncn(C)n2)c1OC] |
| CPD- 095 | 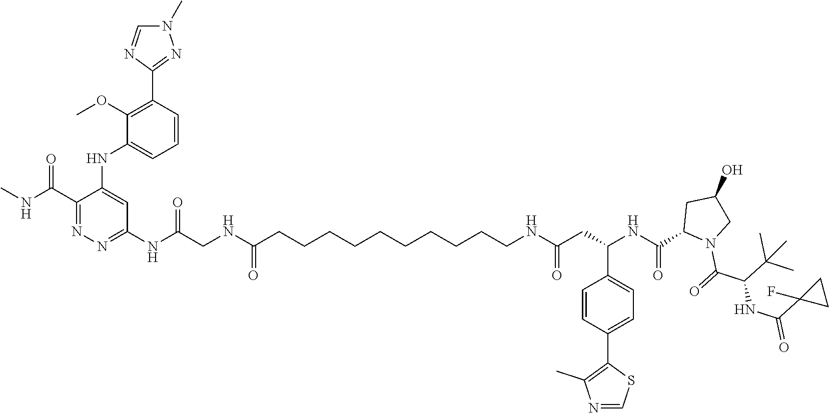 [SMILES: CNC(=O)c1nnc(NC(=O)CNC(=O)CCCCCCCCCCNC(=O)C[C@H](NC(=O)[C@@H]2C[C@@H](O)CN2C(=O)[C@@H](NC(=O)C2(F)CC2)C(C)(C)C)c2ccc(-c3scnc3C)cc2)cc1Nc1cccc(-c2ncn(C)n2)c1OC] |
| CPD- 096 | 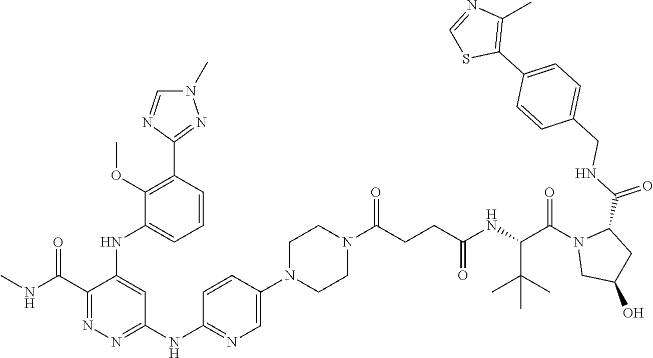 [SMILES: CNC(=O)c1nnc(Nc2ccc(N3CCN(C(=O)CCC(=O)N[C@H](C(=O)N4C[C@H](O)C[C@H]4C(=O)NCc4ccc(-c5scnc5C)cc4)C(C)(C)C)CC3)cn2)cc1Nc1cccc(-c2ncn(C)n2)c1OC] |
| CPD- 097 | 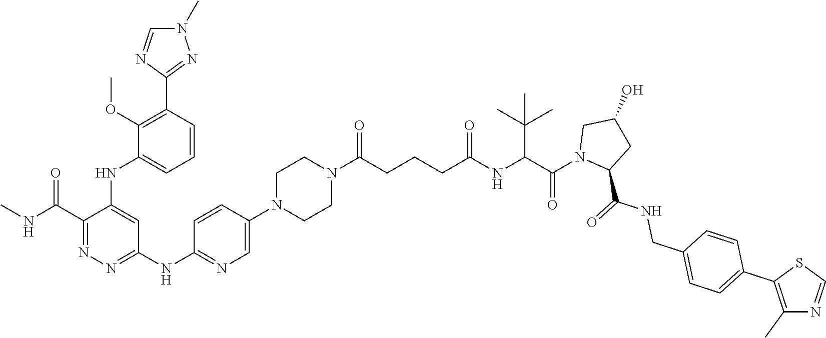 [SMILES: CNC(=O)c1nnc(Nc2ccc(N3CCN(C(=O)CCCC(=O)NC(C(=O)N4C[C@H](O)C[C@H]4C(=O)NCc4ccc(-c5scnc5C)cc4)C(C)(C)C)CC3)cn2)cc1Nc1cccc(-c2ncn(C)n2)c1OC] |
| CPD- 098 | 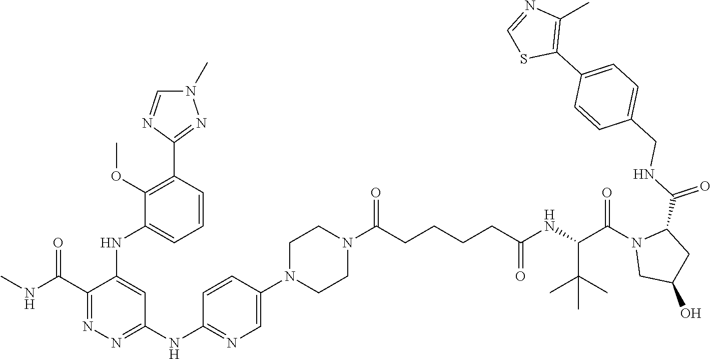 [SMILES: CNC(=O)c1nnc(Nc2ccc(N3CCN(C(=O)CCCCC(=O)N[C@H](C(=O)N4C[C@H](O)C[C@H]4C(=O)NCc4ccc(-c5scnc5C)cc4)C(C)(C)C)CC3)cn2)cc1Nc1cccc(-c2ncn(C)n2)c1OC] |
| CPD- 099 | 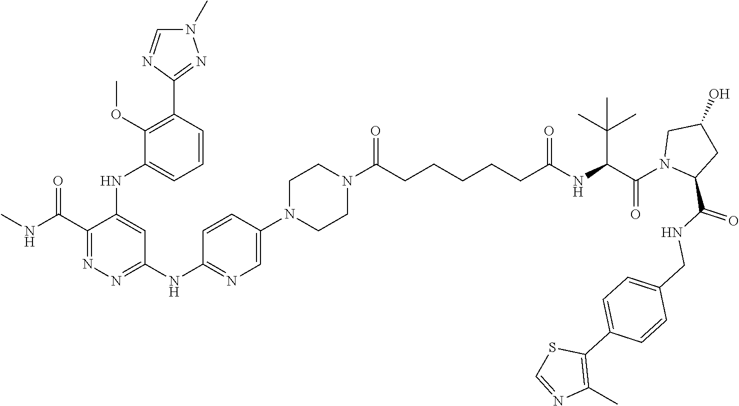 [SMILES: CNC(=O)c1nnc(Nc2ccc(N3CCN(C(=O)CCCCCC(=O)N[C@H](C(=O)N4C[C@H](O)C[C@H]4C(=O)NCc4ccc(-c5scnc5C)cc4)C(C)(C)C)CC3)cn2)cc1Nc1cccc(-c2ncn(C)n2)c1OC] |
| CPD- 100 | 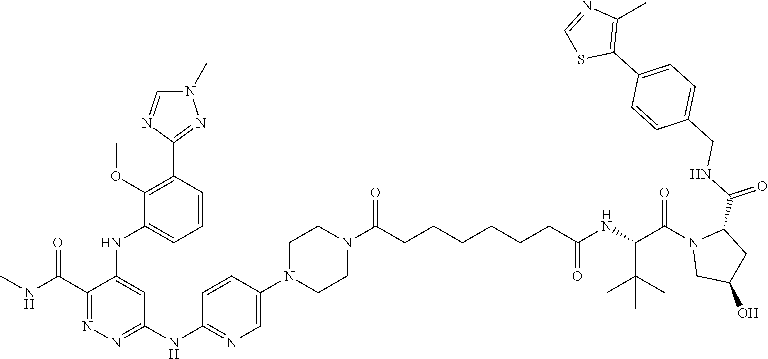 [SMILES: CNC(=O)c1nnc(Nc2ccc(N3CCN(C(=O)CCCCCCC(=O)N[C@H](C(=O)N4C[C@H](O)C[C@H]4C(=O)NCc4ccc(-c5scnc5C)cc4)C(C)(C)C)CC3)cn2)cc1Nc1cccc(-c2ncn(C)n2)c1OC] |
| CPD- 101 | 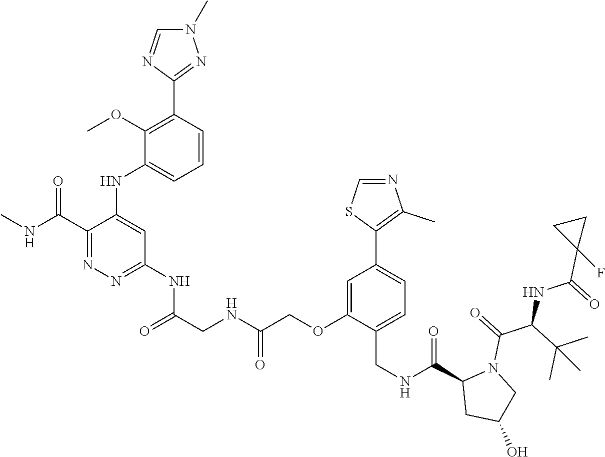 [SMILES: CNC(=O)c1nnc(NC(=O)CNC(=O)COc2cc(-c3scnc3C)ccc2CNC(=O)[C@@H]2C[C@@H](O)CN2C(=O)[C@@H](NC(=O)C2(F)CC2)C(C)(C)C)cc1Nc1cccc(-c2ncn(C)n2)c1OC] |
| CPD- 102 | 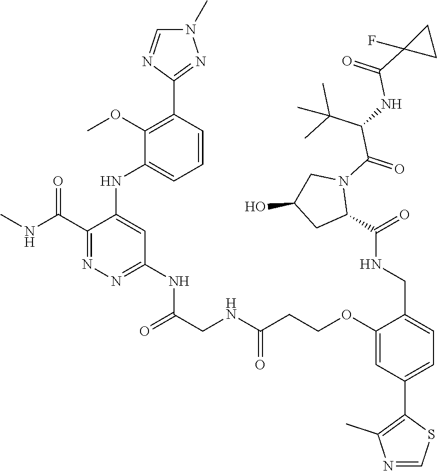 [SMILES: CNC(=O)c1nnc(NC(=O)CNC(=O)CCOc2cc(-c3scnc3C)ccc2CNC(=O)[C@@H]2C[C@@H](O)CN2C(=O)[C@@H](NC(=O)C2(F)CC2)C(C)(C)C)cc1Nc1cccc(-c2ncn(C)n2)c1OC] |
| CPD- 103 | 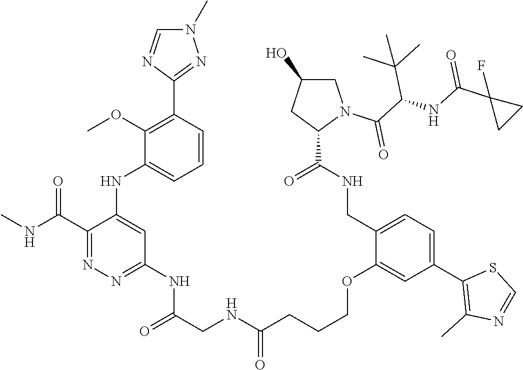 [SMILES: CNC(=O)c1nnc(NC(=O)CNC(=O)CCCOc2cc(-c3scnc3C)ccc2CNC(=O)[C@@H]2C[C@@H](O)CN2C(=O)[C@@H](NC(=O)C2(F)CC2)C(C)(C)C)cc1Nc1cccc(-c2ncn(C)n2)c1OC] |
| CPD- 104 | 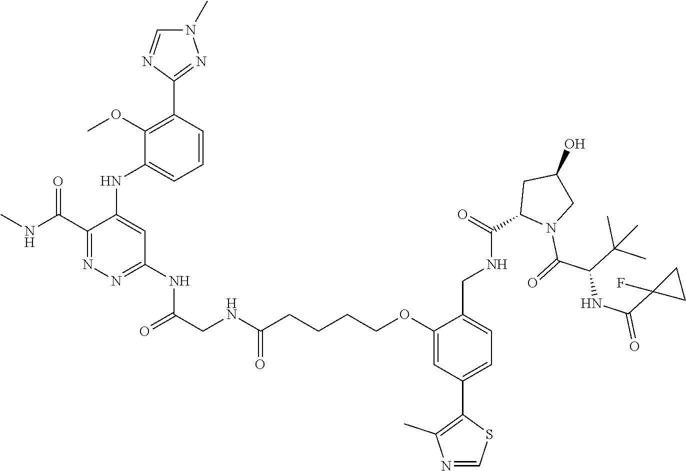 [SMILES: CNC(=O)c1nnc(NC(=O)CNC(=O)CCCCOc2cc(-c3scnc3C)ccc2CNC(=O)[C@@H]2C[C@@H](O)CN2C(=O)[C@@H](NC(=O)C2(F)CC2)C(C)(C)C)cc1Nc1cccc(-c2ncn(C)n2)c1OC] |
| CPD- 105 | 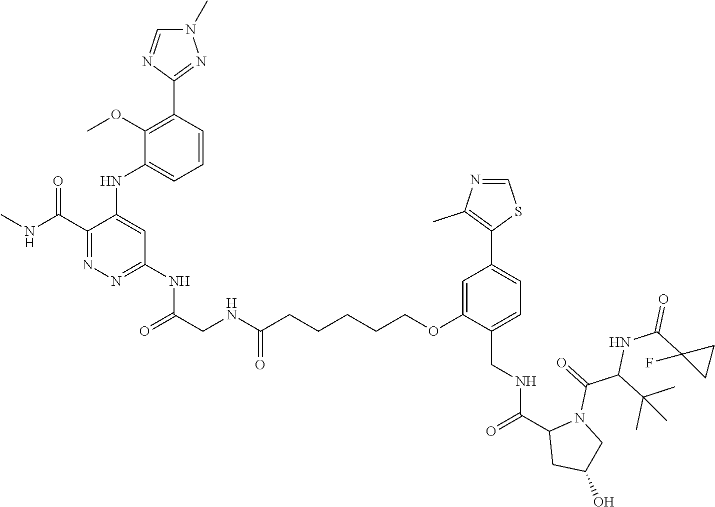 [SMILES: CNC(=O)c1nnc(NC(=O)CNC(=O)CCCCCOc2cc(-c3scnc3C)ccc2CNC(=O)C2C[C@@H](O)CN2C(=O)C(NC(=O)C2(F)CC2)C(C)(C)C)cc1Nc1cccc(-c2ncn(C)n2)c1OC] |
| CPD- 106 | 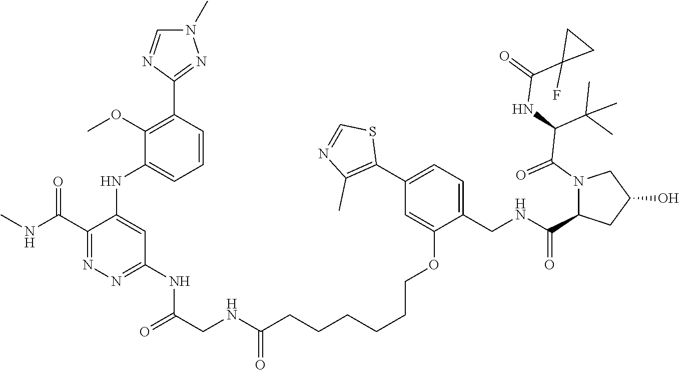 [SMILES: CNC(=O)c1nnc(NC(=O)CNC(=O)CCCCCCOc2cc(-c3scnc3C)ccc2CNC(=O)[C@@H]2C[C@@H](O)CN2C(=O)[C@@H](NC(=O)C2(F)CC2)C(C)(C)C)cc1Nc1cccc(-c2ncn(C)n2)c1OC] |
| CPD- 107 | 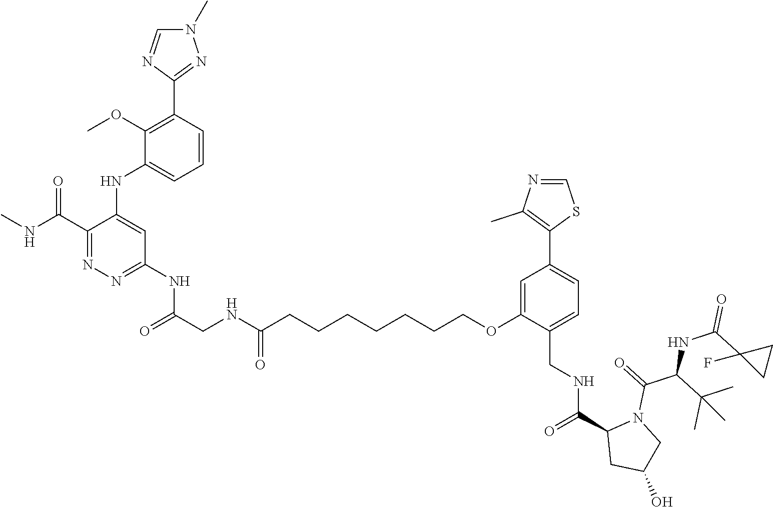 [SMILES: CNC(=O)c1nnc(NC(=O)CNC(=O)CCCCCCCOc2cc(-c3scnc3C)ccc2CNC(=O)[C@@H]2C[C@@H](O)CN2C(=O)[C@@H](NC(=O)C2(F)CC2)C(C)(C)C)cc1Nc1cccc(-c2ncn(C)n2)c1OC] |
| CPD- 108 | 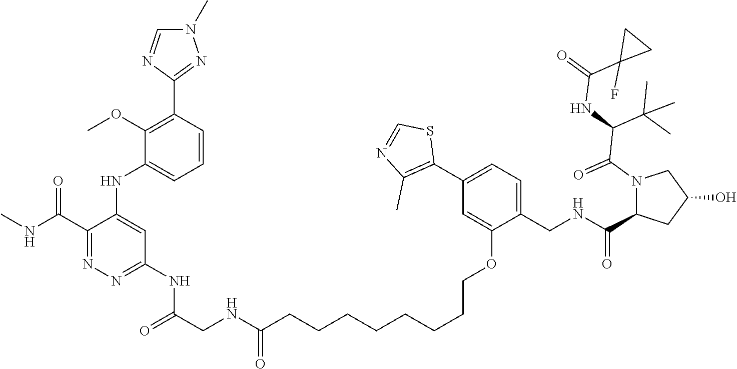 [SMILES: CNC(=O)c1nnc(NC(=O)CNC(=O)CCCCCCCCOc2cc(-c3scnc3C)ccc2CNC(=O)[C@@H]2C[C@@H](O)CN2C(=O)[C@@H](NC(=O)C2(F)CC2)C(C)(C)C)cc1Nc1cccc(-c2ncn(C)n2)c1OC] |
| CPD- 109 | 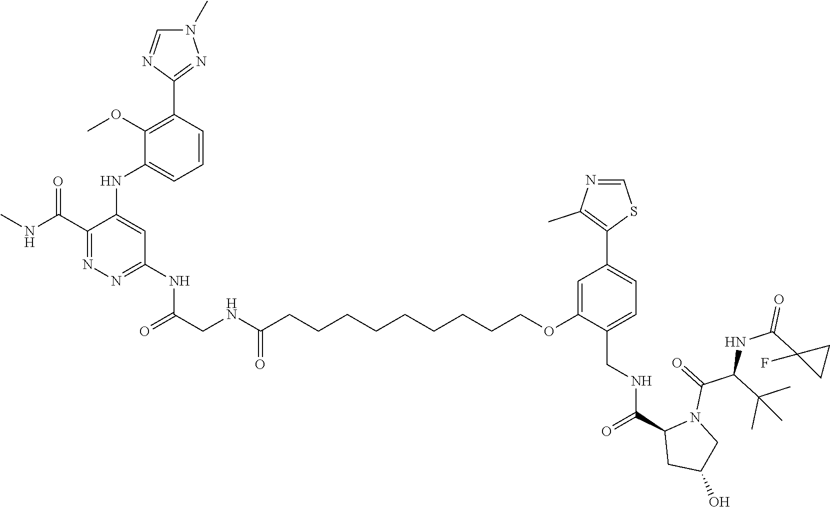 [SMILES: CNC(=O)c1nnc(NC(=O)CNC(=O)CCCCCCCCCOc2cc(-c3scnc3C)ccc2CNC(=O)[C@@H]2C[C@@H](O)CN2C(=O)[C@@H](NC(=O)C2(F)CC2)C(C)(C)C)cc1Nc1cccc(-c2ncn(C)n2)c1OC] |
| CPD- 110 | 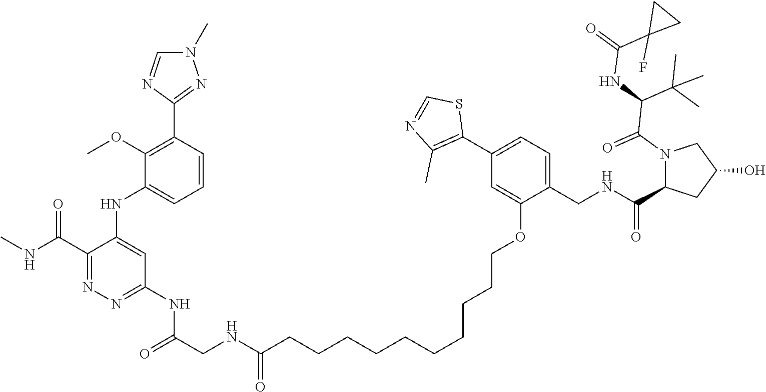 [SMILES: CNC(=O)c1nnc(NC(=O)CNC(=O)CCCCCCCCCCOc2cc(-c3scnc3C)ccc2CNC(=O)[C@@H]2C[C@@H](O)CN2C(=O)[C@@H](NC(=O)C2(F)CC2)C(C)(C)C)cc1Nc1cccc(-c2ncn(C)n2)c1OC] |
| CPD- 111 | 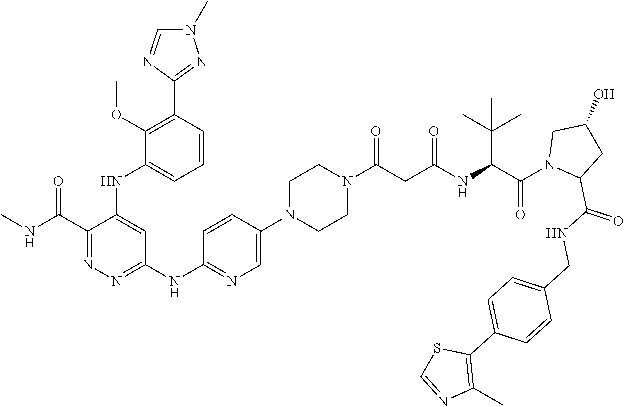 [SMILES: CNC(=O)c1nnc(Nc2ccc(N3CCN(C(=O)CC(=O)N[C@H](C(=O)N4C[C@H](O)CC4C(=O)NCc4ccc(-c5scnc5C)cc4)C(C)(C)C)CC3)cn2)cc1Nc1cccc(-c2ncn(C)n2)c1OC] |
| CPD- 112 | 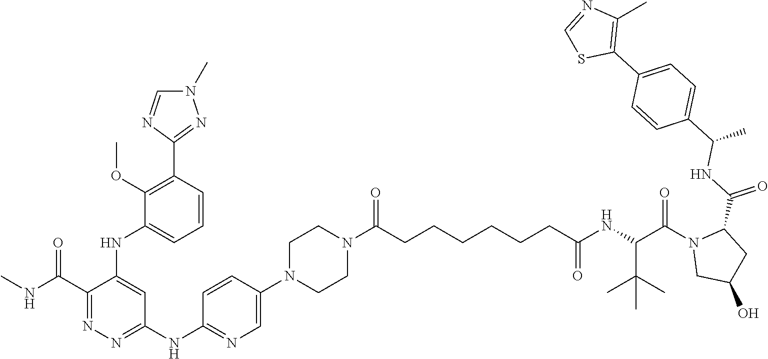 |
| CPD- 113 | 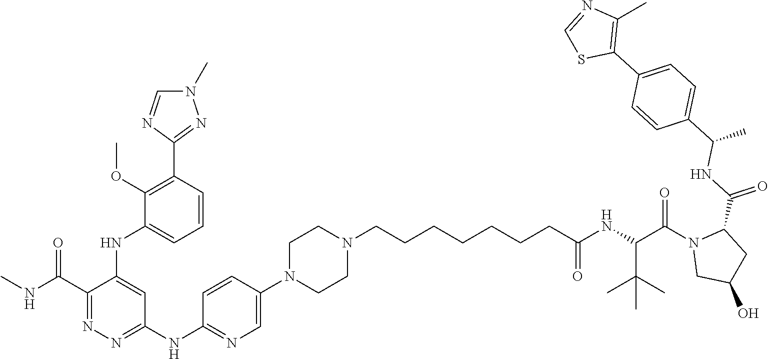 |
| CPD- 114 | 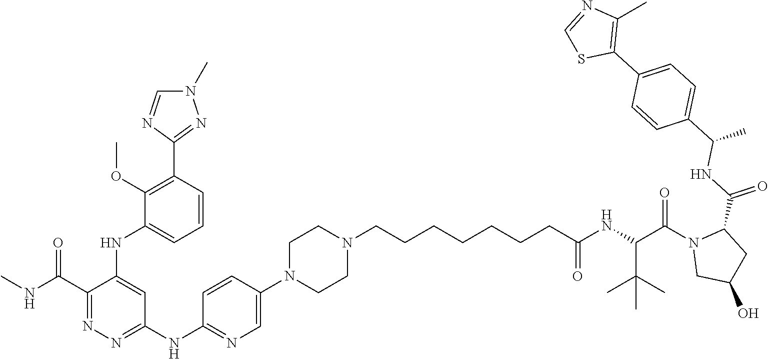 |
| CPD- 115 | 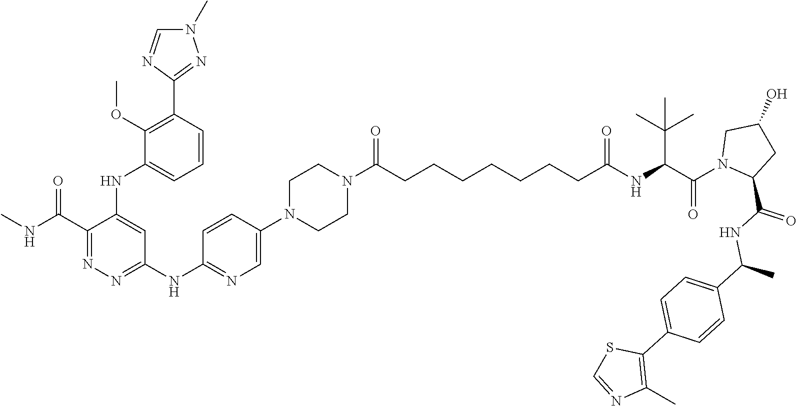 [SMILES: CNC(=O)c1nnc(Nc2ccc(N3CCN(C(=O)CCCCCCCC(=O)N[C@H](C(=O)N4C[C@H](O)C[C@H]4C(=O)N[C@@H](C)c4ccc(-c5scnc5C)cc4)C(C)(C)C)CC3)cn2)cc1Nc1cccc(-c2ncn(C)n2)c1OC] |
| CPD- 116 | 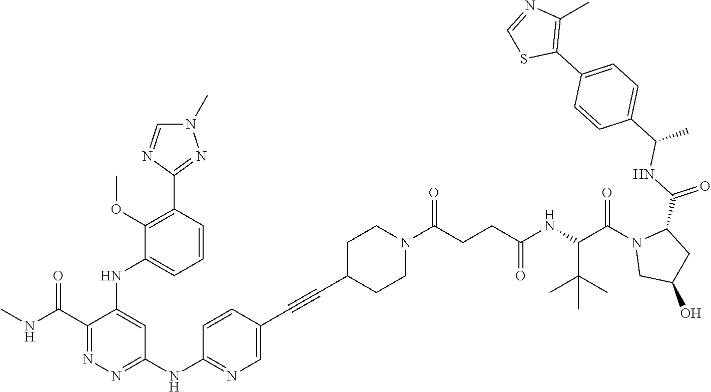 |
| CPD- 117 | 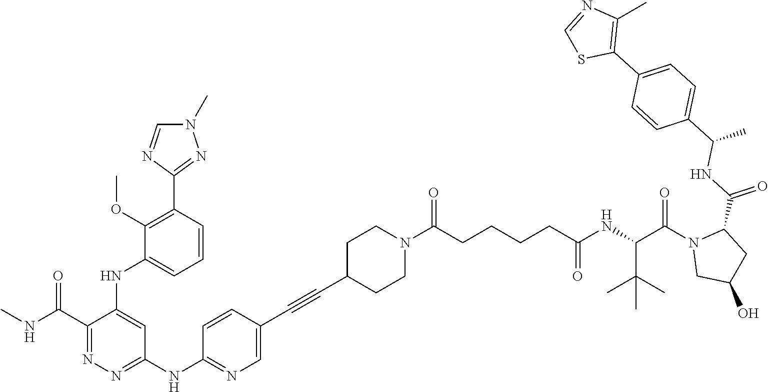 [SMILES: CNC(=O)c1nnc(Nc2ccc(C#CC3CCN(C(=O)CCCCC(=O)N[C@H](C(=O)N4C[C@H](O)C[C@H]4C(=O)N[C@@H](C)c4ccc(-c5scnc5C)cc4)C(C)(C)C)CC3)cn2)cc1Nc1cccc(-c2ncn(C)n2)c1OC] |
| CPD- 118 | 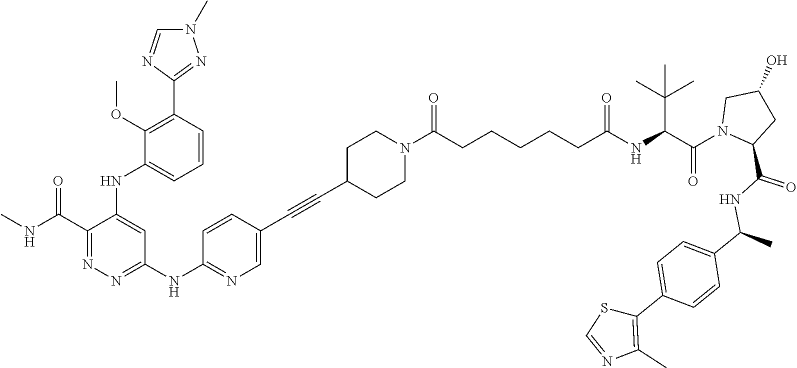 [SMILES: CNC(=O)c1nnc(Nc2ccc(C#CC3CCN(C(=O)CCCCCC(=O)N[C@H](C(=O)N4C[C@H](O)C[C@H]4C(=O)N[C@@H](C)c4ccc(-c5scnc5C)cc4)C(C)(C)C)CC3)cn2)cc1Nc1cccc(-c2ncn(C)n2)c1OC] |
| CPD- 119 | 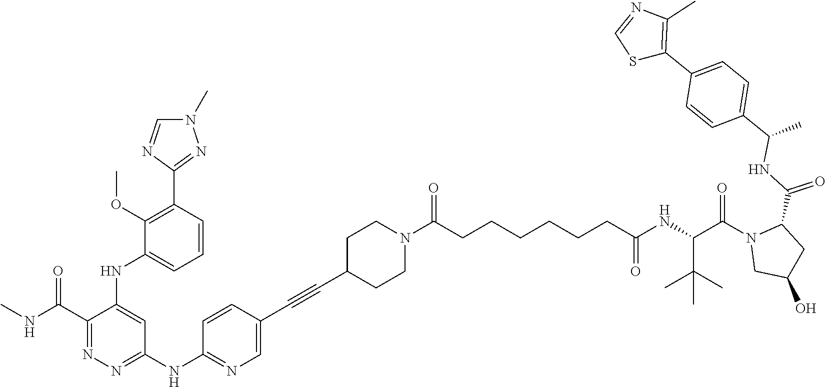 [SMILES: CNC(=O)c1nnc(Nc2ccc(C#CC3CCN(C(=O)CCCCCCC(=O)N[C@H](C(=O)N4C[C@H](O)C[C@H]4C(=O)N[C@@H](C)c4ccc(-c5scnc5C)cc4)C(C)(C)C)CC3)cn2)cc1Nc1cccc(-c2ncn(C)n2)c1OC] |
| CPD- 120 | 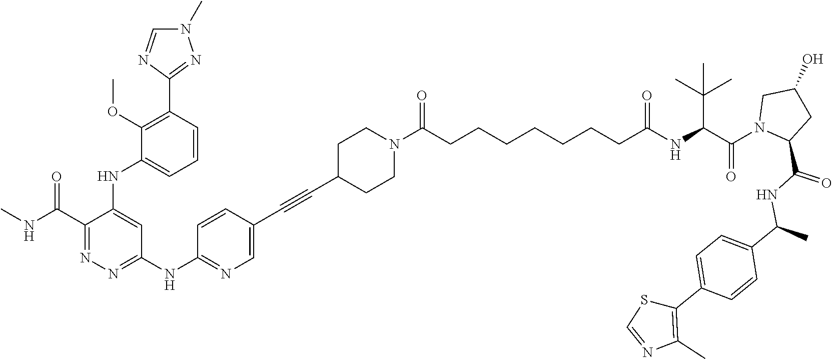 [SMILES: CNC(=O)c1nnc(Nc2ccc(C#CC3CCN(C(=O)CCCCCCCC(=O)N[C@H](C(=O)N4C[C@H](O)C[C@H]4C(=O)N[C@@H](C)c4ccc(-c5scnc5C)cc4)C(C)(C)C)CC3)cn2)cc1Nc1cccc(-c2ncn(C)n2)c1OC] |
| CPD- 121 | 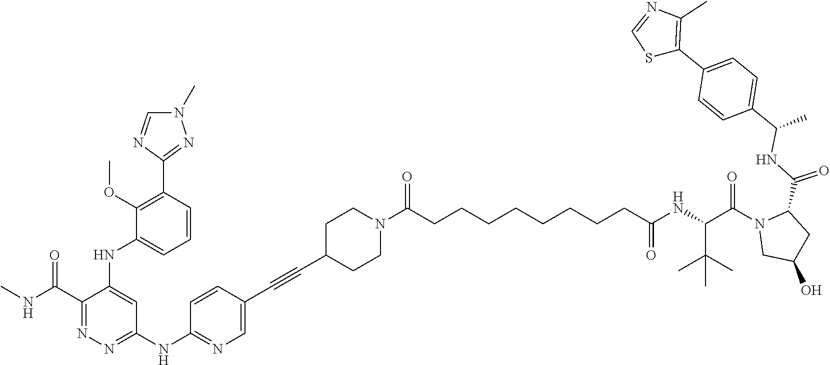 [SMILES: CNC(=O)c1nnc(Nc2ccc(C#CC3CCN(C(=O)CCCCCCCCC(=O)N[C@H](C(=O)N4C[C@H](O)C[C@H]4C(=O)N[C@@H](C)c4ccc(-c5scnc5C)cc4)C(C)(C)C)CC3)cn2)cc1Nc1cccc(-c2ncn(C)n2)c1OC] |
| CPD- 122 | 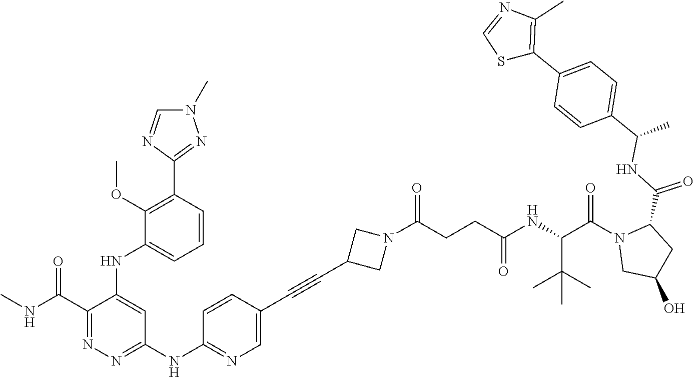 [SMILES: CNC(=O)c1nnc(Nc2ccc(C#CC3CN(C(=O)CCC(=O)N[C@H](C(=O)N4C[C@H](O)C[C@H]4C(=O)N[C@@H](C)c4ccc(-c5scnc5C)cc4)C(C)(C)C)C3)cn2)cc1Nc1cccc(-c2ncn(C)n2)c1OC] |
| CPD- 123 | 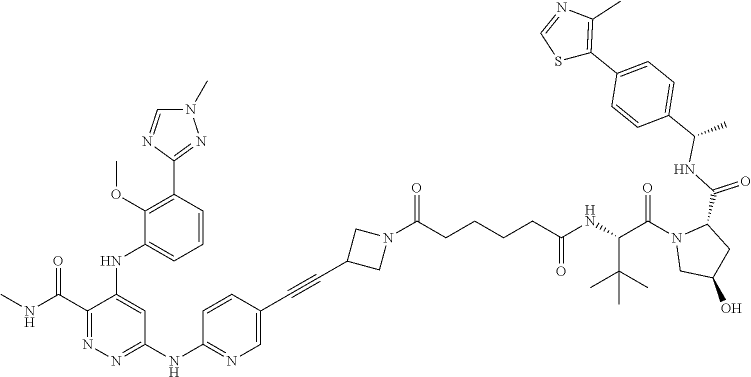 [SMILES: CNC(=O)c1nnc(Nc2ccc(C#CC3CN(C(=O)CCCCC(=O)N[C@H](C(=O)N4C[C@H](O)C[C@H]4C(=O)N[C@@H](C)c4ccc(-c5scnc5C)cc4)C(C)(C)C)C3)cn2)cc1Nc1cccc(-c2ncn(C)n2)c1OC] |
| CPD- 124 | 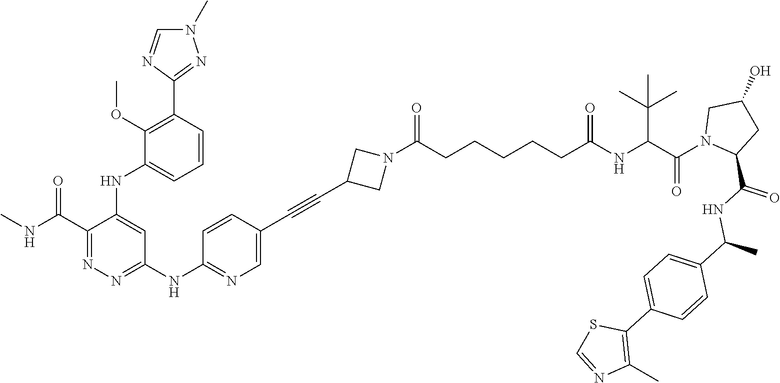 [SMILES: CNC(=O)c1nnc(Nc2ccc(C#CC3CN(C(=O)CCCCCC(=O)NC(C(=O)N4C[C@H](O)C[C@H]4C(=O)N[C@@H](C)c4ccc(-c5scnc5C)cc4)C(C)(C)C)C3)cn2)cc1Nc1cccc(-c2ncn(C)n2)c1OC] |
| CPD- 125 | 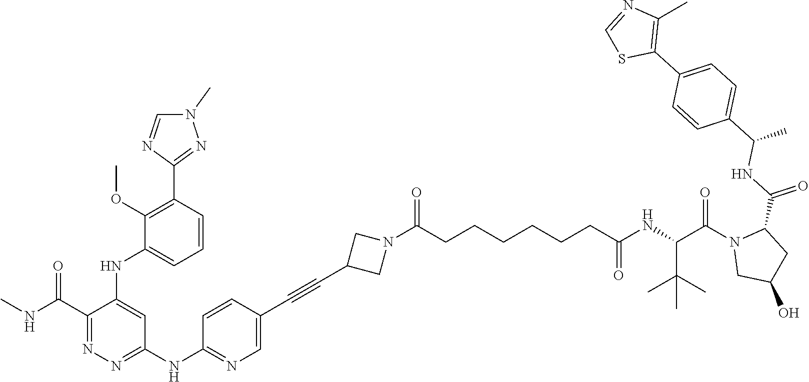 [SMILES: CNC(=O)c1nnc(Nc2ccc(C#CC3CN(C(=O)CCCCCCC(=O)N[C@H](C(=O)N4C[C@H](O)C[C@H]4C(=O)N[C@@H](C)c4ccc(-c5scnc5C)cc4)C(C)(C)C)C3)cn2)cc1Nc1cccc(-c2ncn(C)n2)c1OC] |
| CPD- 126 | 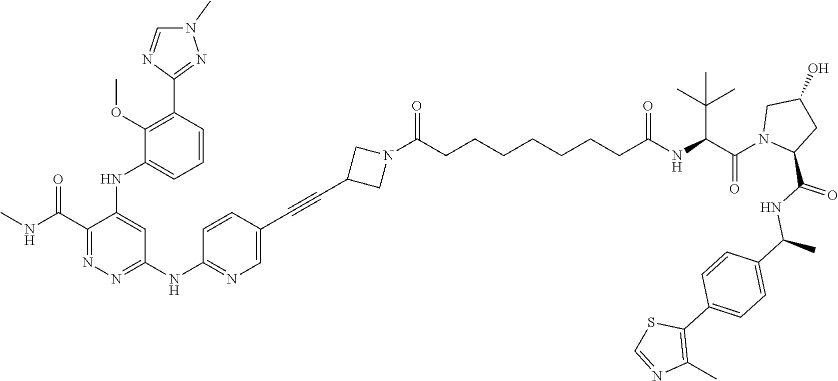 [SMILES: CNC(=O)c1nnc(Nc2ccc(C#CC3CN(C(=O)CCCCCCCC(=O)N[C@H](C(=O)N4C[C@H](O)C[C@H]4C(=O)N[C@@H](C)c4ccc(-c5scnc5C)cc4)C(C)(C)C)C3)cn2)cc1Nc1cccc(-c2ncn(C)n2)c1OC] |
| CPD- 127 | 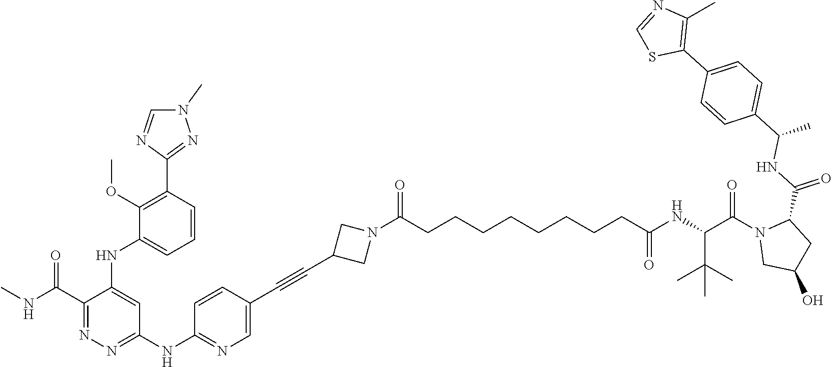 [SMILES: CNC(=O)c1nnc(Nc2ccc(C#CC3CN(C(=O)CCCCCCCCC(=O)N[C@H](C(=O)N4C[C@H](O)C[C@H]4C(=O)N[C@@H](C)c4ccc(-c5scnc5C)cc4)C(C)(C)C)C3)cn2)cc1Nc1cccc(-c2ncn(C)n2)c1OC] |
| CPD- 128 | 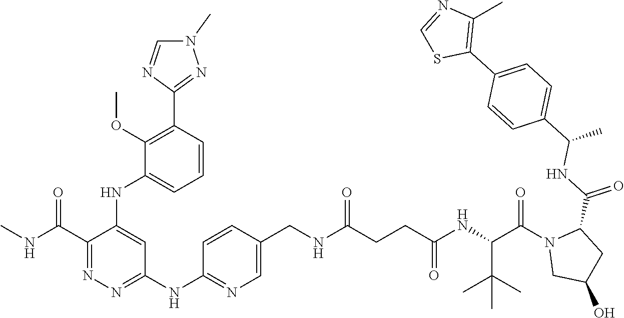 [SMILES: CNC(=O)c1nnc(Nc2ccc(CNC(=O)CCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC] |
| CPD- 129 | 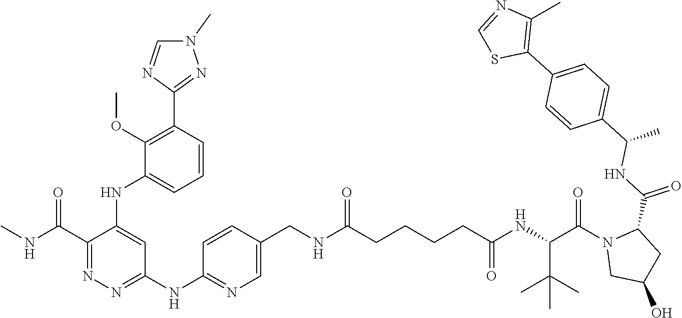 [SMILES: CNC(=O)c1nnc(Nc2ccc(CNC(=O)CCCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC] |
| CPD- 130 | 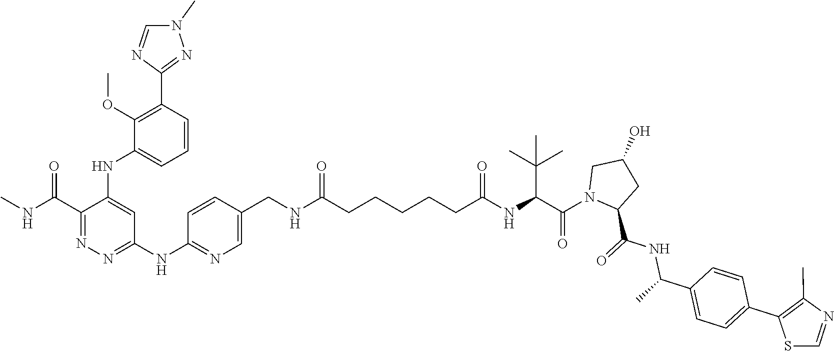 [SMILES: CNC(=O)c1nnc(Nc2ccc(CNC(=O)CCCCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC] |
| CPD- 131 | 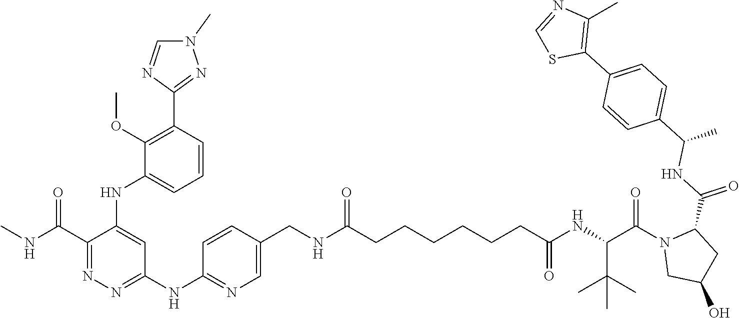 [SMILES: CNC(=O)c1nnc(Nc2ccc(CNC(=O)CCCCCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC] |
| CPD- 132 | 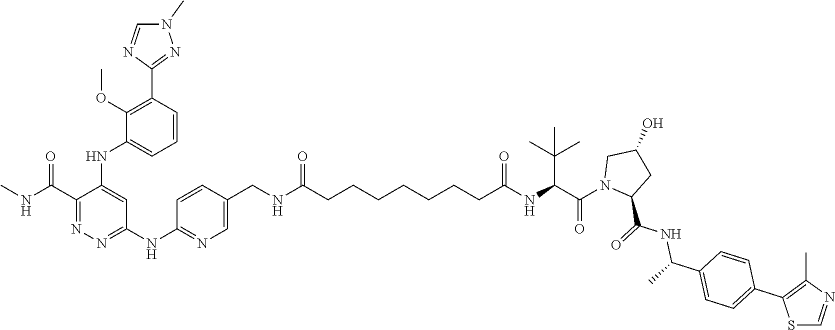 [SMILES: CNC(=O)c1nnc(Nc2ccc(CNC(=O)CCCCCCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC] |
| CPD- 133 | 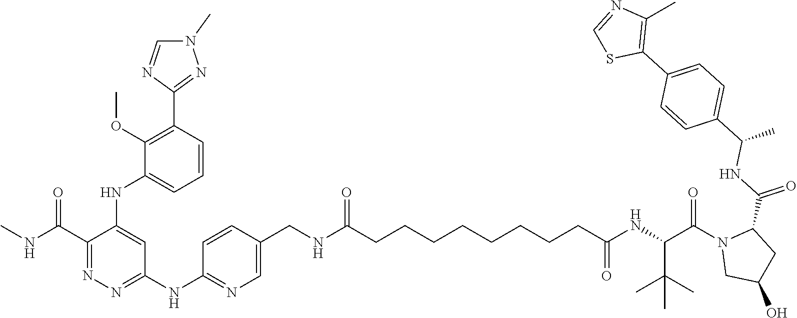 [SMILES: CNC(=O)c1nnc(Nc2ccc(CNC(=O)CCCCCCCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC] |
| CPD- 134 | 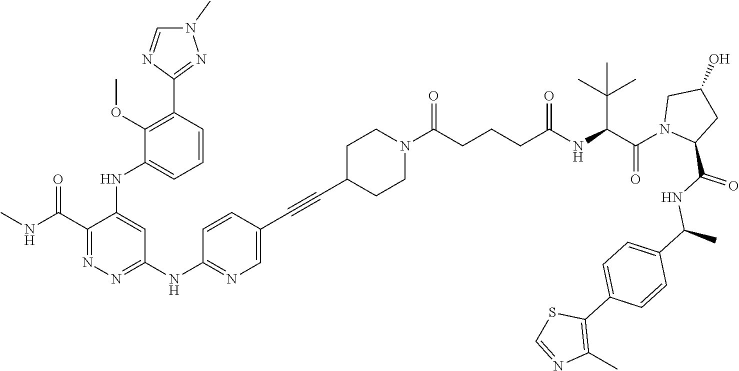 [SMILES: CNC(=O)c1nnc(Nc2ccc(C#CC3CCN(C(=O)CCCC(=O)N[C@H](C(=O)N4C[C@H](O)C[C@H]4C(=O)N[C@@H](C)c4ccc(-c5scnc5C)cc4)C(C)(C)C)CC3)cn2)cc1Nc1cccc(-c2ncn(C)n2)c1OC] |
| CPD- 135 | 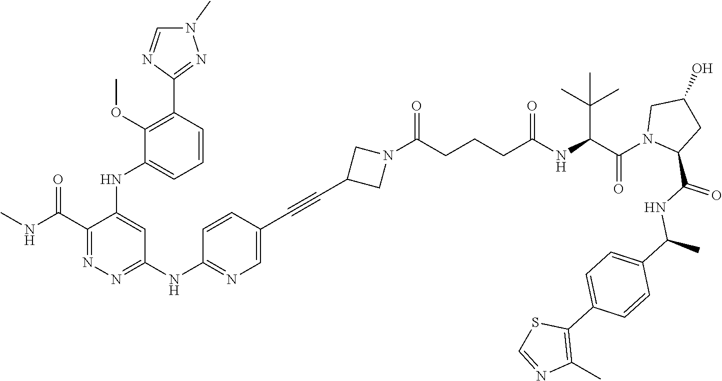 [SMILES: CNC(=O)c1nnc(Nc2ccc(C#CC3CN(C(=O)CCCC(=O)N[C@H](C(=O)N4C[C@H](O)C[C@H]4C(=O)N[C@@H](C)c4ccc(-c5scnc5C)cc4)C(C)(C)C)C3)cn2)cc1Nc1cccc(-c2ncn(C)n2)c1OC] |
| CPD- 136 | 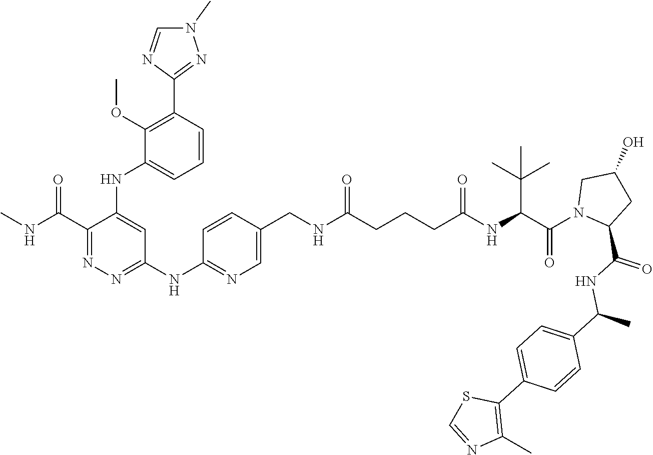 [SMILES: CNC(=O)c1nnc(Nc2ccc(CNC(=O)CCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC] |
| CPD- 137 | 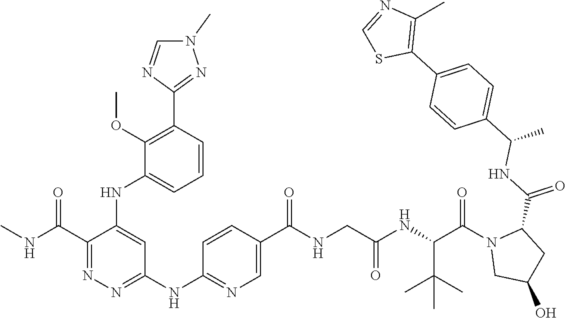 [SMILES: CNC(=O)c1nnc(Nc2ccc(C(=O)NCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC] |
| CPD- 138 | 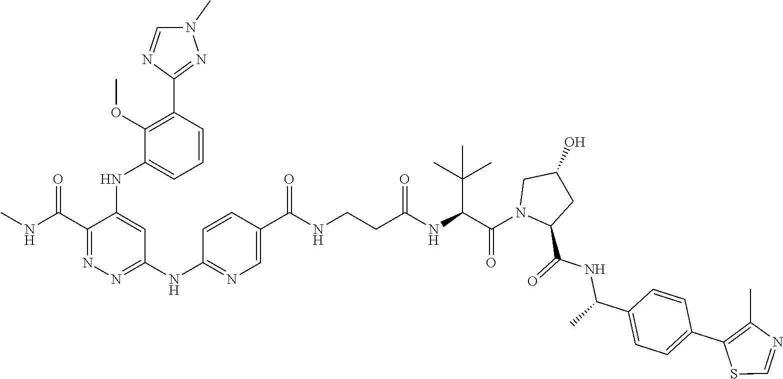 [SMILES: CNC(=O)c1nnc(Nc2ccc(C(=O)NCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC] |
| CPD- 139 | 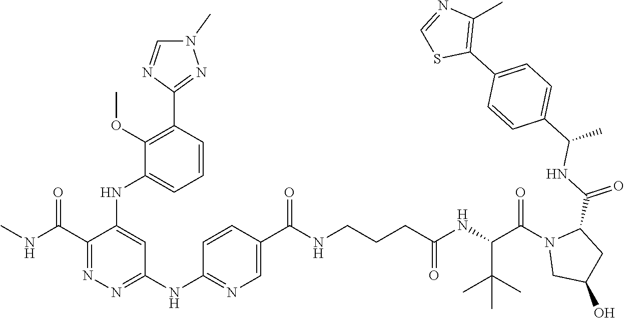 [SMILES: CNC(=O)c1nnc(Nc2ccc(C(=O)NCCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC] |
| CPD- 140 | 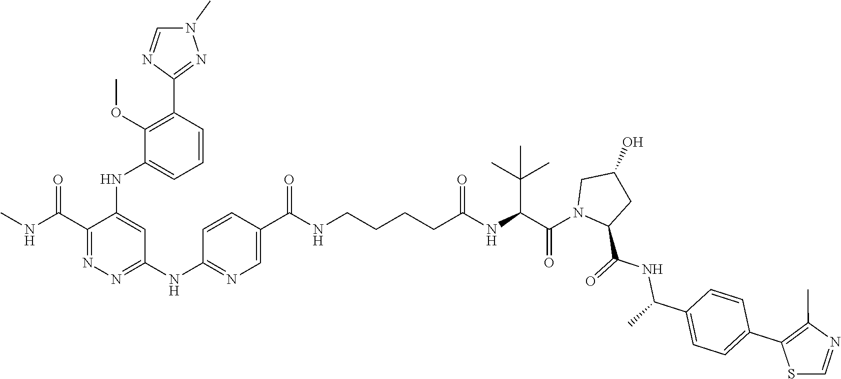 [SMILES: CNC(=O)c1nnc(Nc2ccc(C(=O)NCCCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC] |
| CPD- 141 | 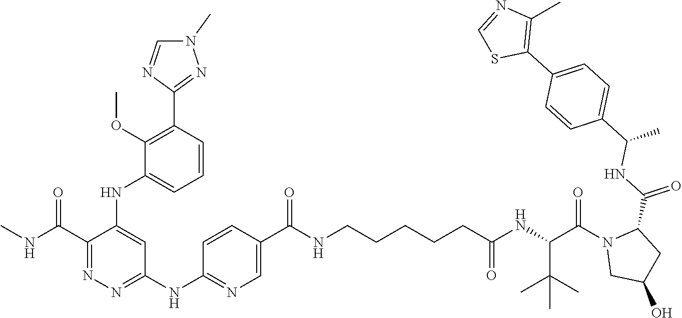 [SMILES: CNC(=O)c1nnc(Nc2ccc(C(=O)NCCCCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC] |
| CPD- 142 | 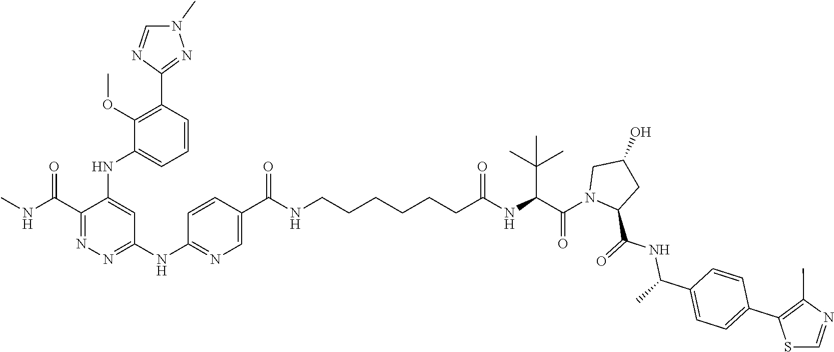 [SMILES: CNC(=O)c1nnc(Nc2ccc(C(=O)NCCCCCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC] |
| CPD- 143 | 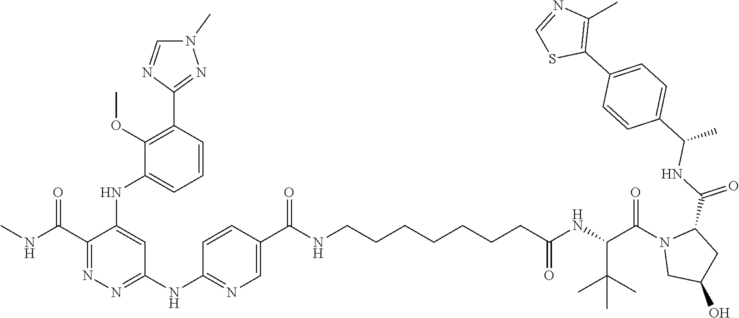 [SMILES: CNC(=O)c1nnc(Nc2ccc(C(=O)NCCCCCCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC] |
| CPD- 144 | 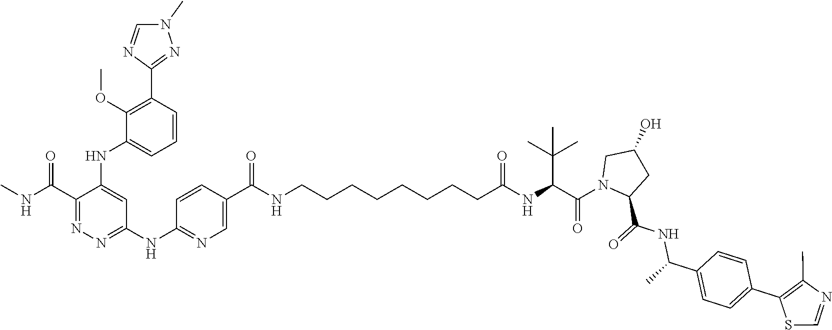 [SMILES: CNC(=O)c1nnc(Nc2ccc(C(=O)NCCCCCCCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC] |
| CPD- 145 | 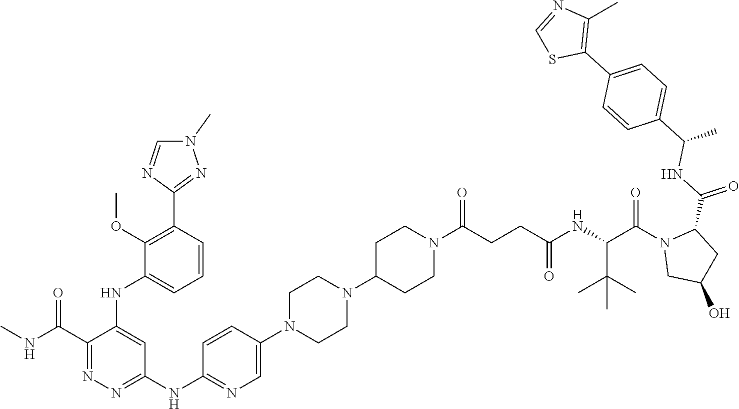 [SMILES: CNC(=O)c1nnc(Nc2ccc(N3CCN(C4CCN(C(=O)CCC(=O)N[C@H](C(=O)N5C[C@H](O)C[C@H]5C(=O)N[C@@H](C)c5ccc(-c6scnc6C)cc5)C(C)(C)C)CC4)CC3)cn2)cc1Nc1cccc(-c2ncn(C)n2)c1OC] |
| CPD- 146 | 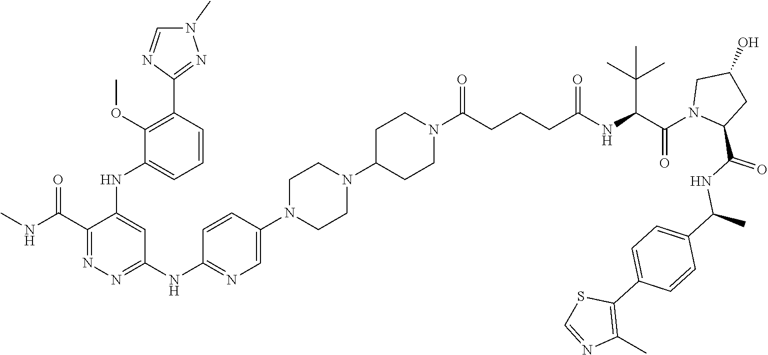 [SMILES: CNC(=O)c1nnc(Nc2ccc(N3CCN(C4CCN(C(=O)CCCC(=O)N[C@H](C(=O)N5C[C@H](O)C[C@H]5C(=O)N[C@@H](C)c5ccc(-c6scnc6C)cc5)C(C)(C)C)CC4)CC3)cn2)cc1Nc1cccc(-c2ncn(C)n2)c1OC] |
| CPD- 147 | 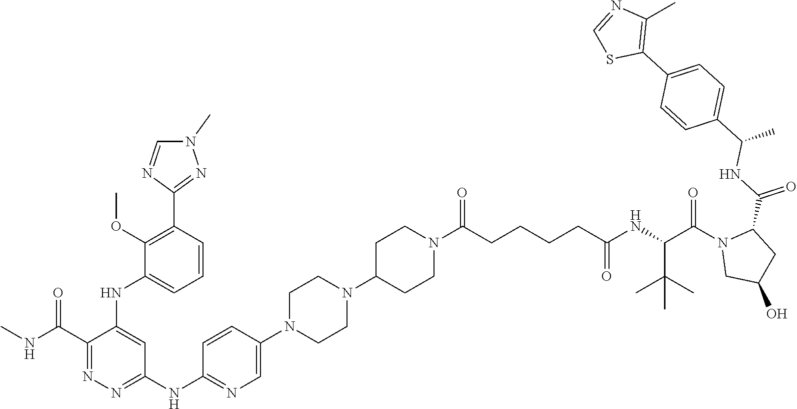 [SMILES: CNC(=O)c1nnc(Nc2ccc(N3CCN(C4CCN(C(=O)CCCCC(=O)N[C@H](C(=O)N5C[C@H](O)C[C@H]5C(=O)N[C@@H](C)c5ccc(-c6scnc6C)cc5)C(C)(C)C)CC4)CC3)cn2)cc1Nc1cccc(-c2ncn(C)n2)c1OC] |
| CPD- 148 | 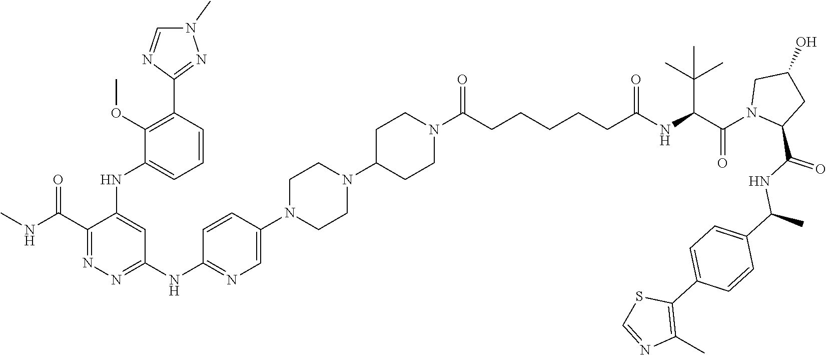 [SMILES: CNC(=O)c1nnc(Nc2ccc(N3CCN(C4CCN(C(=O)CCCCCC(=O)N[C@H](C(=O)N5C[C@H](O)C[C@H]5C(=O)N[C@@H](C)c5ccc(-c6scnc6C)cc5)C(C)(C)C)CC4)CC3)cn2)cc1Nc1cccc(-c2ncn(C)n2)c1OC] |
| CPD- 149 | 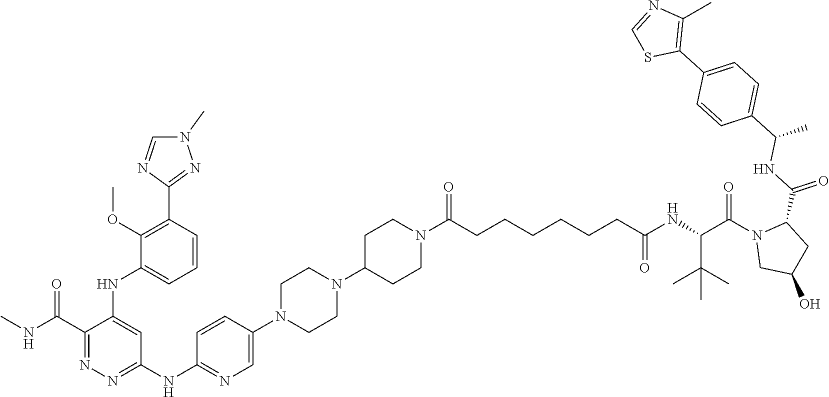 [SMILES: CNC(=O)c1nnc(Nc2ccc(N3CCN(C4CCN(C(=O)CCCCCCC(=O)N[C@H](C(=O)N5C[C@H](O)C[C@H]5C(=O)N[C@@H](C)c5ccc(-c6scnc6C)cc5)C(C)(C)C)CC4)CC3)cn2)cc1Nc1cccc(-c2ncn(C)n2)c1OC] |
| CPD- 150 | 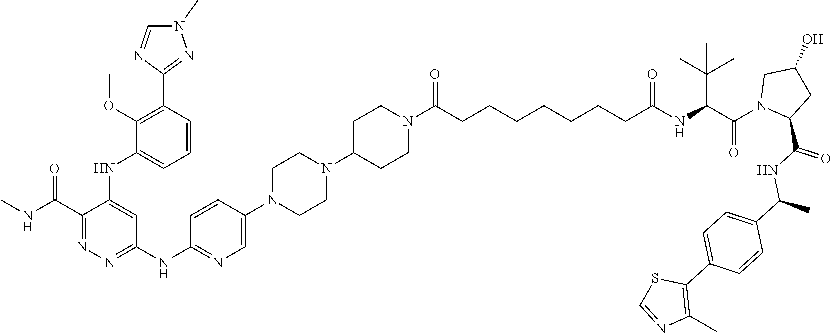 [SMILES: CNC(=O)c1nnc(Nc2ccc(N3CCN(C4CCN(C(=O)CCCCCCCC(=O)N[C@H](C(=O)N5C[C@H](O)C[C@H]5C(=O)N[C@@H](C)c5ccc(-c6scnc6C)cc5)C(C)(C)C)CC4)CC3)cn2)cc1Nc1cccc(-c2ncn(C)n2)c1OC] |
| CPD- 151 | 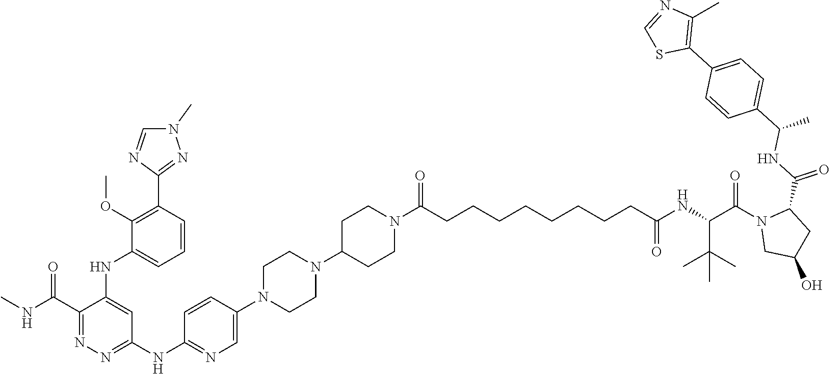 [SMILES: CNC(=O)c1nnc(Nc2ccc(N3CCN(C4CCN(C(=O)CCCCCCCCC(=O)N[C@H](C(=O)N5C[C@H](O)C[C@H]5C(=O)N[C@@H](C)c5ccc(-c6scnc6C)cc5)C(C)(C)C)CC4)CC3)cn2)cc1Nc1cccc(-c2ncn(C)n2)c1OC] |
| CPD- 152 | 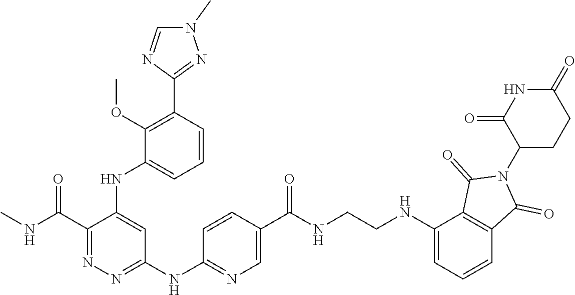 [SMILES: CNC(=O)c1nnc(Nc2ccc(C(=O)NCCNc3cccc4c3C(=O)N(C3CCC(=O)NC3=O)C4=O)cn2)cc1Nc1cccc(-c2ncn(C)n2)c1OC] |
| CPD- 153 | 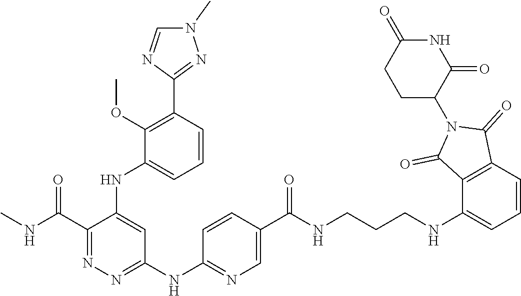 [SMILES: CNC(=O)c1nnc(Nc2ccc(C(=O)NCCCNc3cccc4c3C(=O)N(C3CCC(=O)NC3=O)C4=O)cn2)cc1Nc1cccc(-c2ncn(C)n2)c1OC] |
| CPD- 154 | 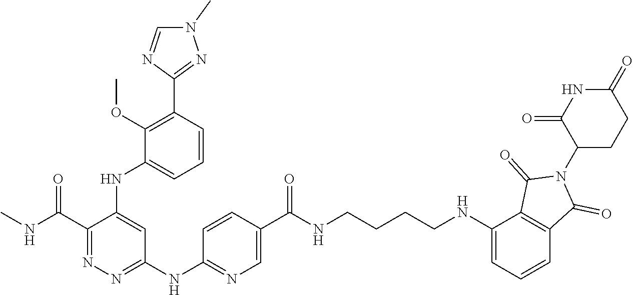 [SMILES: CNC(=O)c1nnc(Nc2ccc(C(=O)NCCCCNc3cccc4c3C(=O)N(C3CCC(=O)NC3=O)C4=O)cn2)cc1Nc1cccc(-c2ncn(C)n2)c1OC] |
| CPD- 155 | 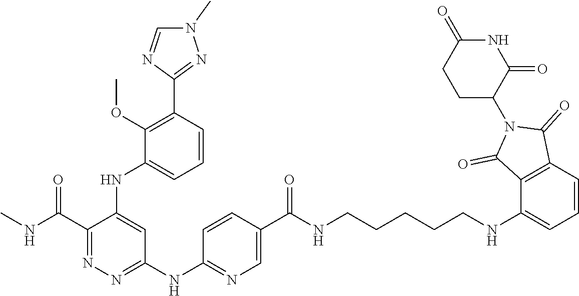 [SMILES: CNC(=O)c1nnc(Nc2ccc(C(=O)NCCCCCNc3cccc4c3C(=O)N(C3CCC(=O)NC3=O)C4=O)cn2)cc1Nc1cccc(-c2ncn(C)n2)c1OC] |
| CPD- 156 | 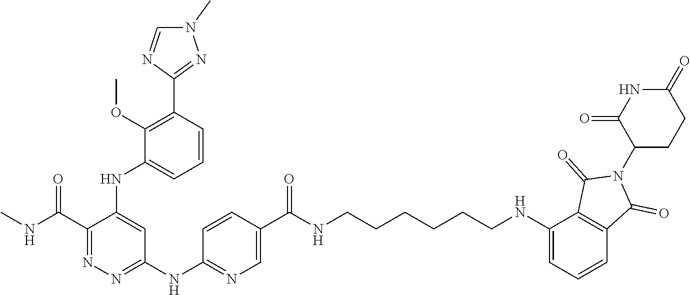 [SMILES: CNC(=O)c1nnc(Nc2ccc(C(=O)NCCCCCCNc3cccc4c3C(=O)N(C3CCC(=O)NC3=O)C4=O)cn2)cc1Nc1cccc(-c2ncn(C)n2)c1OC] |
| CPD- 157 | 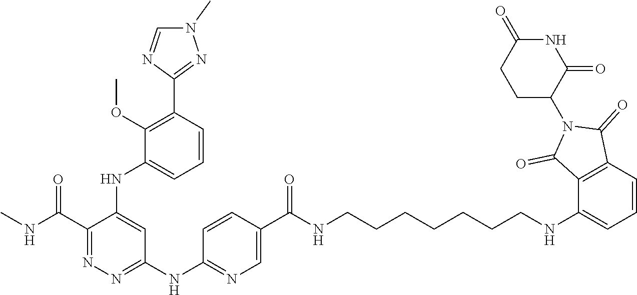 [SMILES: CNC(=O)c1nnc(Nc2ccc(C(=O)NCCCCCCCNc3cccc4c3C(=O)N(C3CCC(=O)NC3=O)C4=O)cn2)cc1Nc1cccc(-c2ncn(C)n2)c1OC] |
| CPD- 158 | 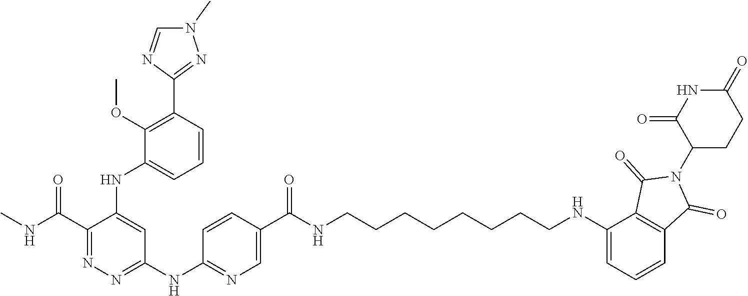 [SMILES: CNC(=O)c1nnc(Nc2ccc(C(=O)NCCCCCCCCNc3cccc4c3C(=O)N(C3CCC(=O)NC3=O)C4=O)cn2)cc1Nc1cccc(-c2ncn(C)n2)c1OC] |
| CPD- 159 | 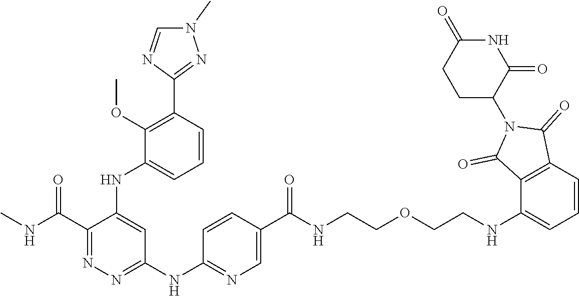 [SMILES: CNC(=O)c1nnc(Nc2ccc(C(=O)NCCOCCNc3cccc4c3C(=O)N(C3CCC(=O)NC3=O)C4=O)cn2)cc1Nc1cccc(-c2ncn(C)n2)c1OC] |
| CPD- 160 | 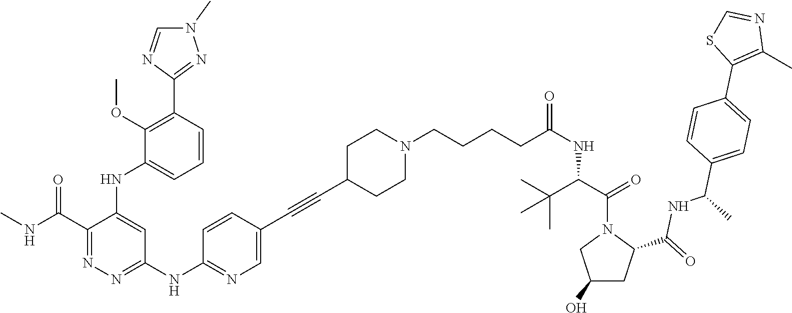 |
| CPD- 161 | 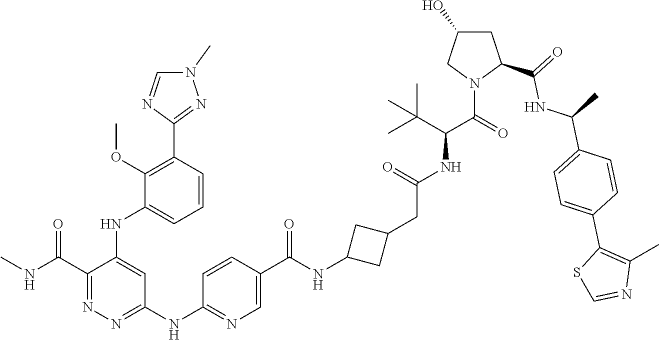 |
| CPD- 162 | 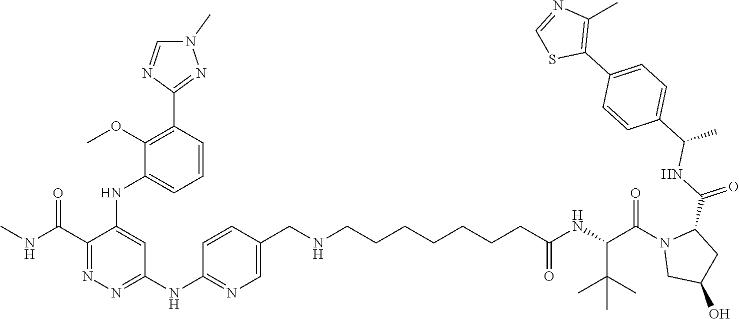 [SMILES: CNC(=O)c1nnc(Nc2ccc(CNCCCCCCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC] |
| CPD- 163 | 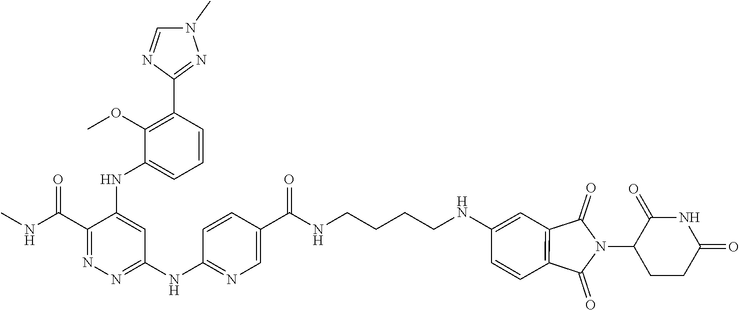 [SMILES: CNC(=O)c1nnc(Nc2ccc(C(=O)NCCCCNc3ccc4c(c3)C(=O)N(C3CCC(=O)NC3=O)C4=O)cn2)cc1Nc1cccc(-c2ncn(C)n2)c1OC] |
| CPD- 164 | 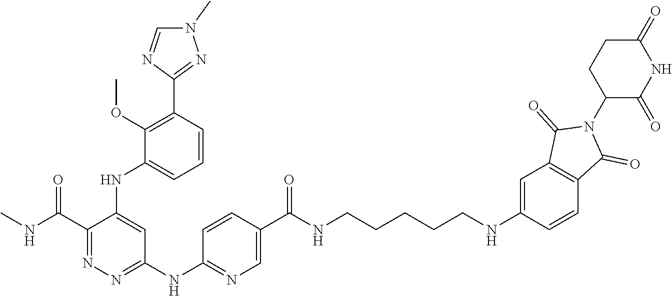 [SMILES: CNC(=O)c1nnc(Nc2ccc(C(=O)NCCCCCNc3ccc4c(c3)C(=O)N(C3CCC(=O)NC3=O)C4=O)cn2)cc1Nc1cccc(-c2ncn(C)n2)c1OC] |
| CPD- 165 | 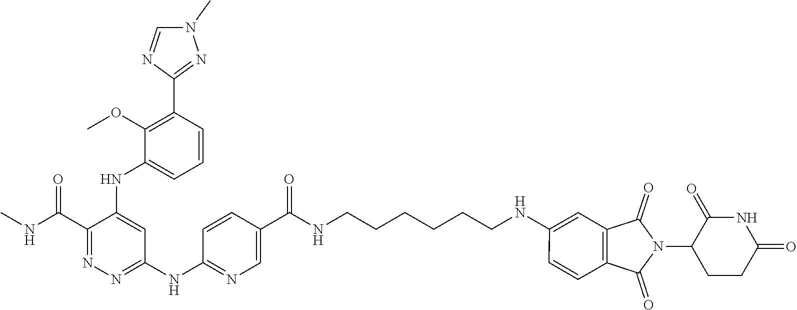 [SMILES: CNC(=O)c1nnc(Nc2ccc(C(=O)NCCCCCCNc3ccc4c(c3)C(=O)N(C3CCC(=O)NC3=O)C4=O)cn2)cc1Nc1cccc(-c2ncn(C)n2)c1OC] |
| CPD- 166 | 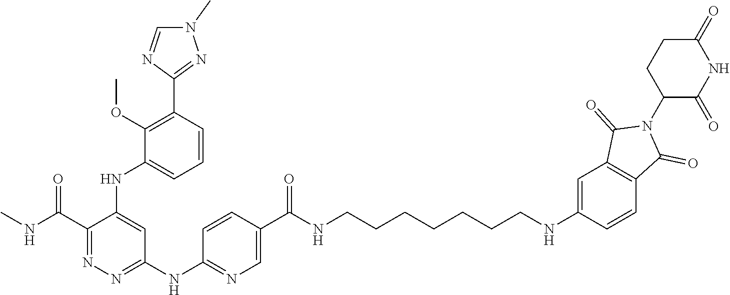 [SMILES: CNC(=O)c1nnc(Nc2ccc(C(=O)NCCCCCCCNc3ccc4c(c3)C(=O)N(C3CCC(=O)NC3=O)C4=O)cn2)cc1Nc1cccc(-c2ncn(C)n2)c1OC] |
| CPD- 167 | 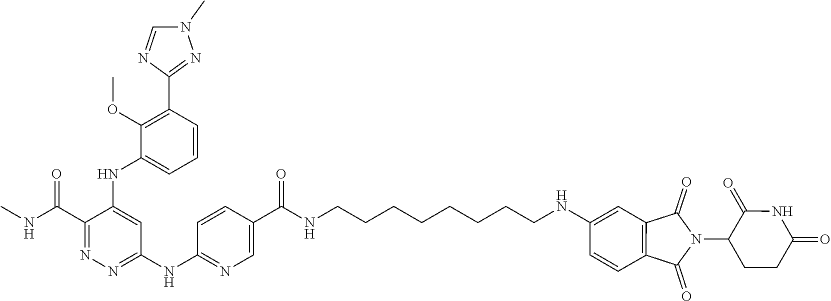 [SMILES: CNC(=O)c1nnc(Nc2ccc(C(=O)NCCCCCCCCNc3ccc4c(c3)C(=O)N(C3CCC(=O)NC3=O)C4=O)cn2)cc1Nc1cccc(-c2ncn(C)n2)c1OC] |
| CPD- 168 | 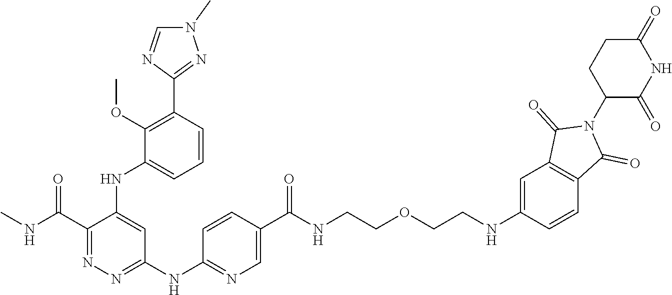 [SMILES: CNC(=O)c1nnc(Nc2ccc(C(=O)NCCOCCNc3ccc4c(c3)C(=O)N(C3CCC(=O)NC3=O)C4=O)cn2)cc1Nc1cccc(-c2ncn(C)n2)c1OC] |
| CPD- 169 | 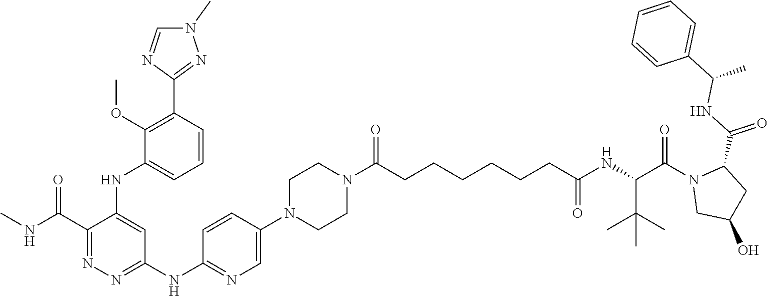 [SMILES: CNC(=O)c1nnc(Nc2ccc(N3CCN(C(=O)CCCCCCC(=O)N[C@H](C(=O)N4C[C@H](O)C[C@H]4C(=O)N[C@@H](C)c4ccccc4)C(C)(C)C)CC3)cn2)cc1Nc1cccc(-c2ncn(C)n2)c1OC] |
| CPD- 170 | 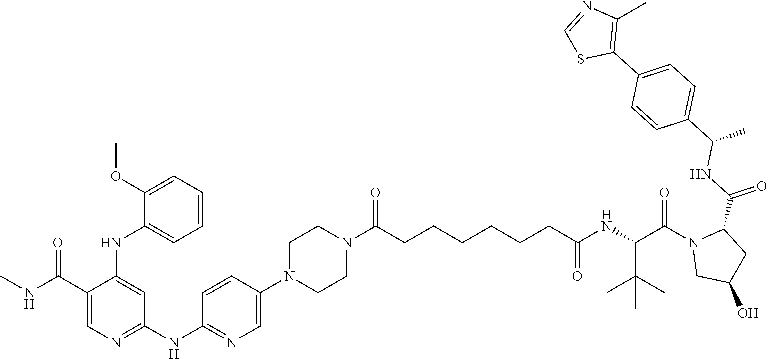 [SMILES: CNC(=O)c1cnc(Nc2ccc(N3CCN(C(=O)CCCCCCC(=O)N[C@H](C(=O)N4C[C@H](O)C[C@H]4C(=O)N[C@@H](C)c4ccc(-c5scnc5C)cc4)C(C)(C)C)CC3)cn2)cc1Nc1ccccc1OC] |
| CPD- 171 | 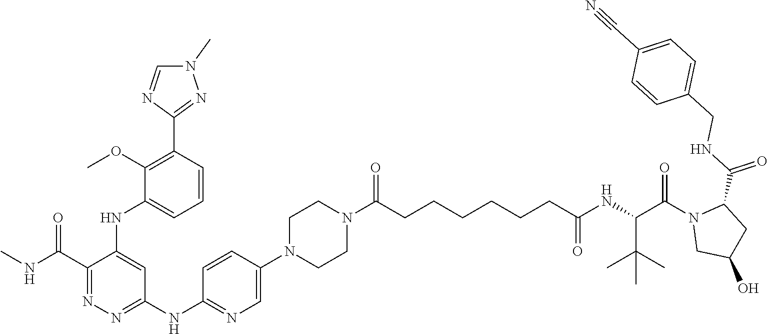 [SMILES: CNC(=O)c1nnc(Nc2ccc(N3CCN(C(=O)CCCCCCC(=O)N[C@H](C(=O)N4C[C@H](O)C[C@H]4C(=O)NCc4ccc(C#N)cc4)C(C)(C)C)CC3)cn2)cc1Nc1cccc(-c2ncn(C)n2)c1OC] |
| CPD- 172 | 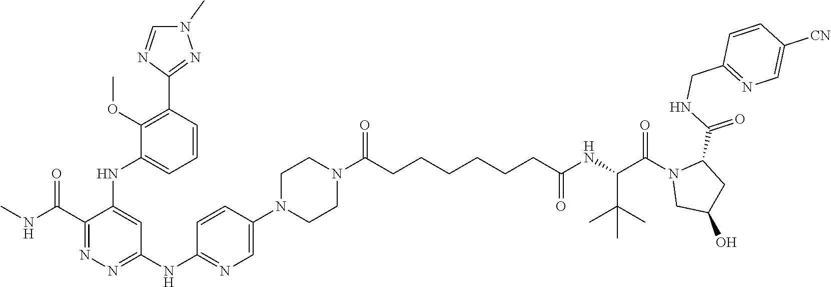 [SMILES: CNC(=O)c1nnc(Nc2ccc(N3CCN(C(=O)CCCCCCC(=O)N[C@H](C(=O)N4C[C@H](O)C[C@H]4C(=O)NCc4ccc(C#N)cn4)C(C)(C)C)CC3)cn2)cc1Nc1cccc(-c2ncn(C)n2)c1OC] |
| CPD -173 | 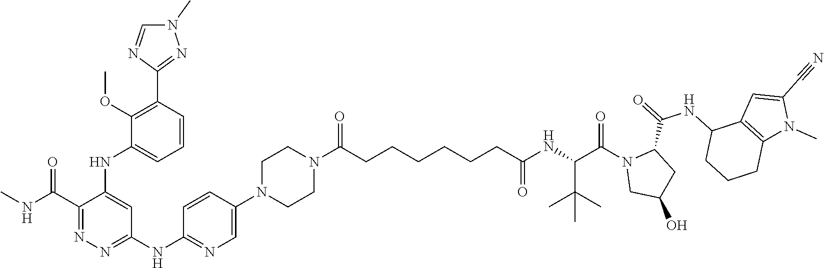 [SMILES: CNC(=O)c1nnc(Nc2ccc(N3CCN(C(=O)CCCCCCC(=O)N[C@H](C(=O)N4C[C@H](O)C[C@H]4C(=O)NC4CCCc5c4cc(C#N)n5C)C(C)(C)C)CC3)cn2)cc1Nc1cccc(-c2ncn(C)n2)c1OC] |
| CPD- 174 | 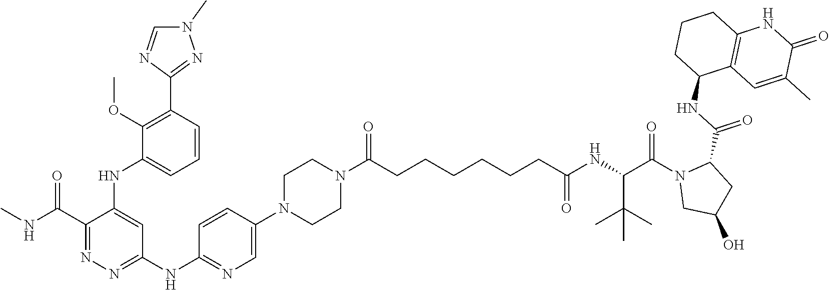 [SMILES: CNC(=O)c1nnc(Nc2ccc(N3CCN(C(=O)CCCCCCC(=O)N[C@H](C(=O)N4C[C@H](O)C[C@H]4C(=O)N[C@H]4CCCc5[nH]c(=O)c(C)cc54)C(C)(C)C)CC3)cn2)cc1Nc1cccc(-c2ncn(C)n2)c1OC] |
| CPD- 175 | 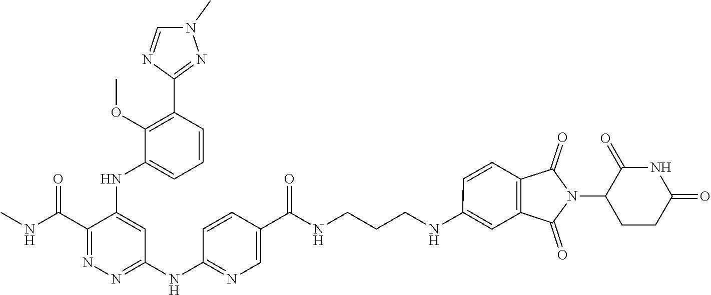 [SMILES: CNC(=O)c1nnc(Nc2ccc(C(=O)NCCCNc3ccc4c(c3)C(=O)N(C3CCC(=O)NC3=O)C4=O)cn2)cc1Nc1cccc(-c2ncn(C)n2)c1OC] |
| CPD- 176 | 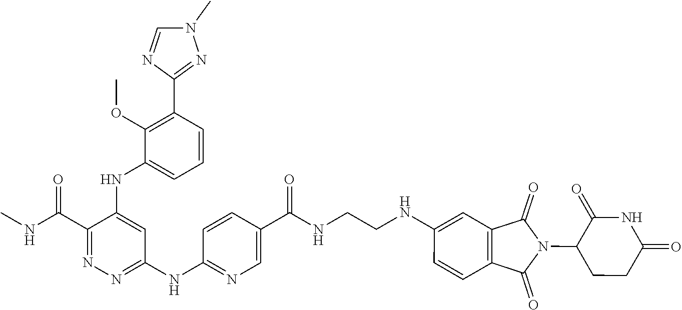 [SMILES: CNC(=O)c1nnc(Nc2ccc(C(=O)NCCNc3ccc4c(c3)C(=O)N(C3CCC(=O)NC3=O)C4=O)cn2)cc1Nc1cccc(-c2ncn(C)n2)c1OC] |
| CPD- 177 | 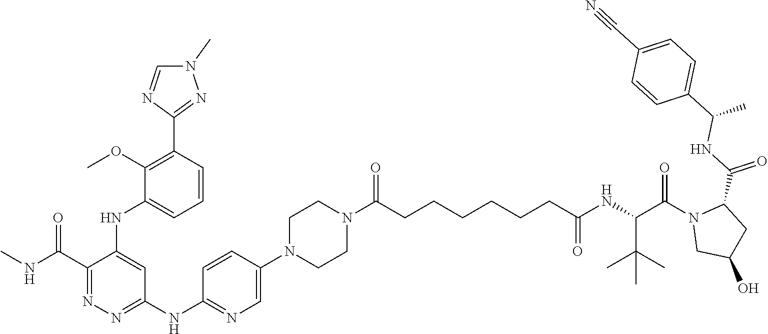 [SMILES: CNC(=O)c1nnc(Nc2ccc(N3CCN(C(=O)CCCCCCC(=O)N[C@H](C(=O)N4C[C@H](O)C[C@H]4C(=O)N[C@@H](C)c4ccc(C#N)cc4)C(C)(C)C)CC3)cn2)cc1Nc1cccc(-c2ncn(C)n2)c1OC] |
| CPD- 178 | 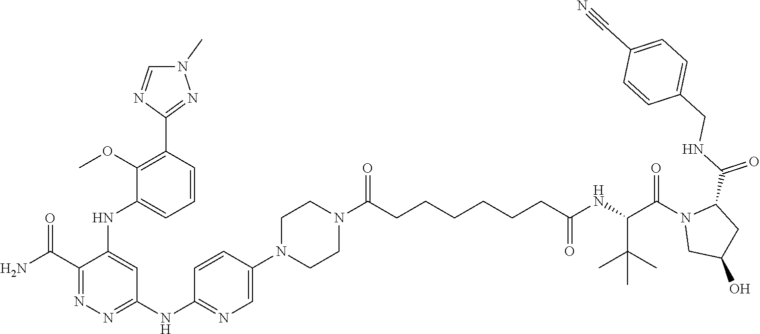 [SMILES: COc1c(Nc2cc(Nc3ccc(N4CCN(C(=O)CCCCCCC(=O)N[C@H](C(=O)N5C[C@H](O)C[C@H]5C(=O)NCc5ccc(C#N)cc5)C(C)(C)C)CC4)cn3)nnc2C(N)=O)cccc1-c1ncn(C)n1] |
| CPD- 179 | 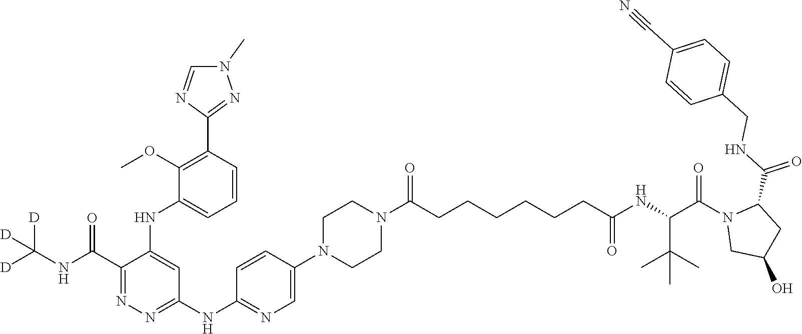 [SMILES: [2H]C([2H])([2H])NC(=O)c1nnc(Nc2ccc(N3CCN(C(=O)CCCCCCC(=O)N[C@H](C(=O)N4C[C@H](O)C[C@H]4C(=O)NCc4ccc(C#N)cc4)C(C)(C)C)CC3)cn2)cc1Nc1cccc(-c2ncn(C)n2)c1OC] |
| CPD- 180 | 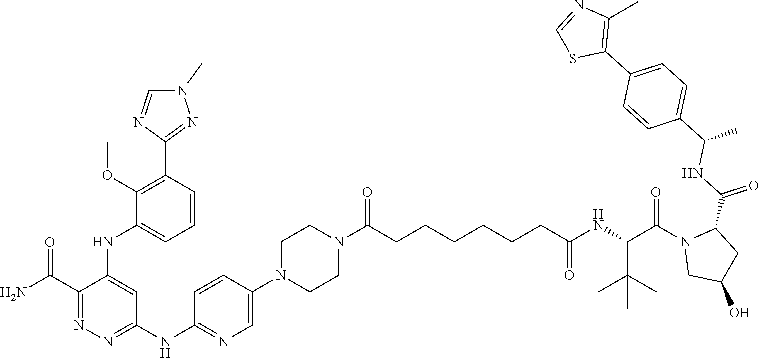 [SMILES: COc1c(Nc2cc(Nc3ccc(N4CCN(C(=O)CCCCCCC(=O)N[C@H](C(=O)N5C[C@H](O)C[C@H]5C(=O)N[C@@H](C)c5ccc(-c6scnc6C)cc5)C(C)(C)C)CC4)cn3)nnc2C(N)=O)cccc1-c1ncn(C)n1] |
| CPD- 181 | 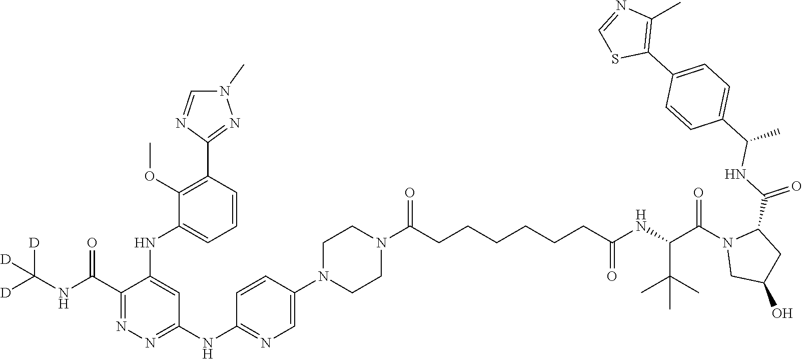 [SMILES: [2H]C([2H])([2H])NC(=O)c1nnc(Nc2ccc(N3CCN(C(=O)CCCCCCC(=O)N[C@H](C(=O)N4C[C@H](O)C[C@H]4C(=O)N[C@@H](C)c4ccc(-c5scnc5C)cc4)C(C)(C)C)CC3)cn2)cc1Nc1cccc(-c2ncn(C)n2)c1OC] |
| CPD- 182 | 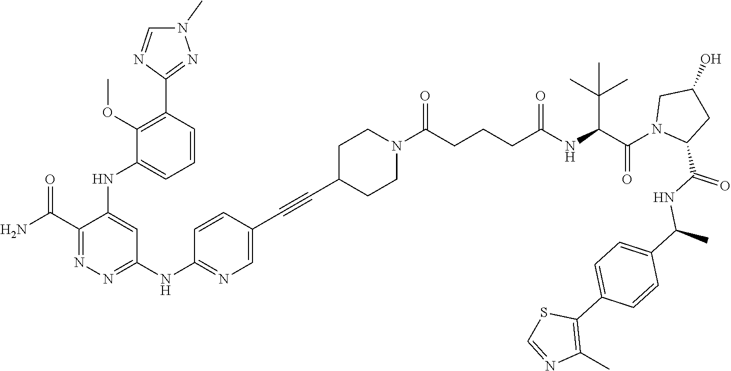 [SMILES: COc1c(Nc2cc(Nc3ccc(C#CC4CCN(C(=O)CCCC(=O)N[C@H](C(=O)N5C[C@H](O)C[C@@H]5C(=O)N[C@@H](C)c5ccc(-c6scnc6C)cc5)C(C)(C)C)CC4)cn3)nnc2C(N)=O)cccc1-c1ncn(C)n1] |
| CPD- 183 | 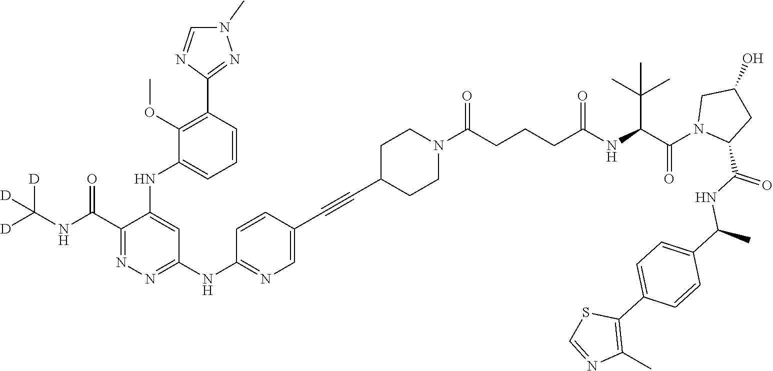 [SMILES: [2H]C([2H])([2H])NC(=O)c1nnc(Nc2ccc(C#CC3CCN(C(=O)CCCC(=O)N[C@H](C(=O)N4C[C@H](O)C[C@@H]4C(=O)N[C@@H](C)c4ccc(-c5scnc5C)cc4)C(C)(C)C)CC3)cn2)cc1Nc1cccc(-c2ncn(C)n2)c1OC] |
| CPD- 184 | 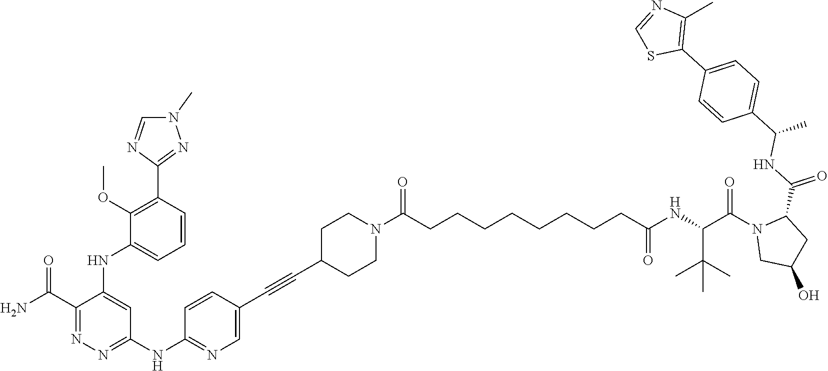 [SMILES: COc1c(Nc2cc(Nc3ccc(C#CC4CCN(C(=O)CCCCCCCCC(=O)N[C@H](C(=O)N5C[C@H](O)C[C@H]5C(=O)N[C@@H](C)c5ccc(-c6scnc6C)cc5)C(C)(C)C)CC4)cn3)nnc2C(N)=O)cccc1-c1ncn(C)n1] |
| CPD- 185 | 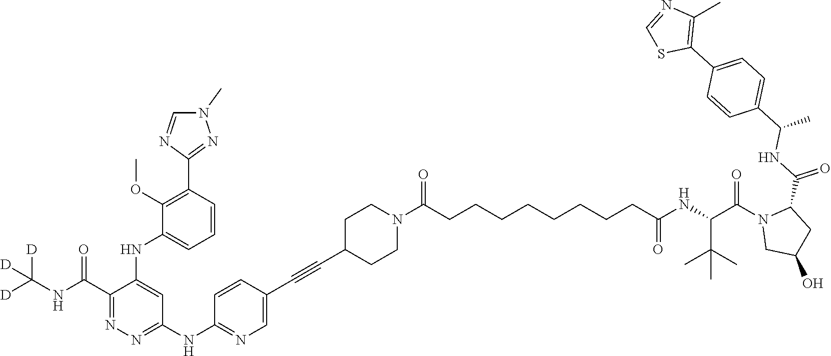 [SMILES: [2H]C([2H])([2H])NC(=O)c1nnc(Nc2ccc(C#CC3CCN(C(=O)CCCCCCCCC(=O)N[C@H](C(=O)N4C[C@H](O)C[C@H]4C(=O)N[C@@H](C)c4ccc(-c5scnc5C)cc4)C(C)(C)C)CC3)cn2)cc1Nc1cccc(-c2ncn(C)n2)c1OC] |
| CPD- 186 | 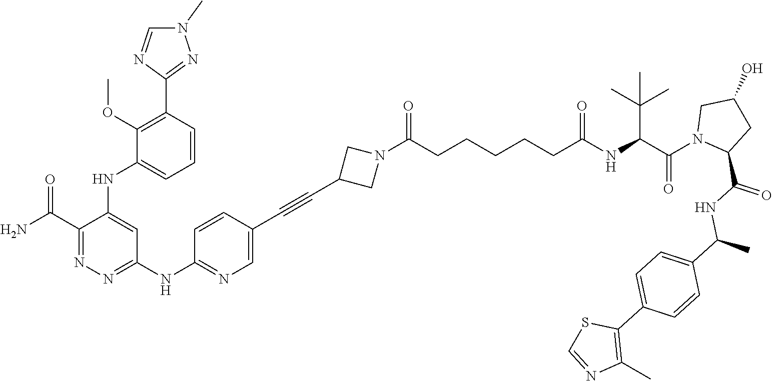 [SMILES: COc1c(Nc2cc(Nc3ccc(C#CC4CN(C(=O)CCCCCC(=O)N[C@H](C(=O)N5C[C@H](O)C[C@H]5C(=O)N[C@@H](C)c5ccc(-c6scnc6C)cc5)C(C)(C)C)C4)cn3)nnc2C(N)=O)cccc1-c1ncn(C)n1] |
| CPD- 187 | 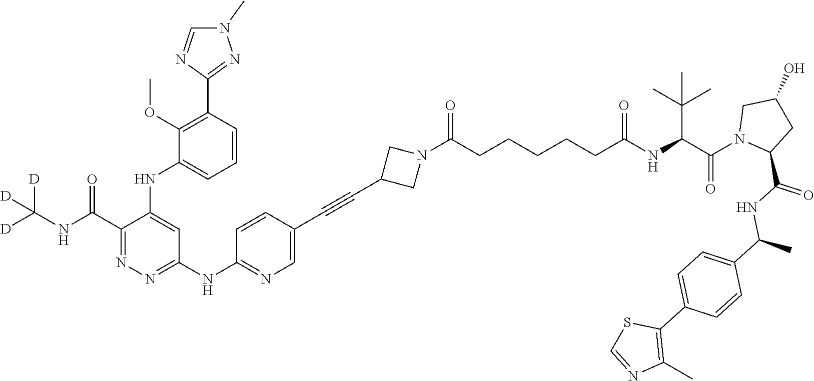 [SMILES: [2H]C([2H])([2H])NC(=O)c1nnc(Nc2ccc(C#CC3CN(C(=O)CCCCCC(=O)N[C@H](C(=O)N4C[C@H](O)C[C@H]4C(=O)N[C@@H](C)c4ccc(-c5scnc5C)cc4)C(C)(C)C)C3)cn2)cc1Nc1cccc(-c2ncn(C)n2)c1OC] |
| CPD- 188 | 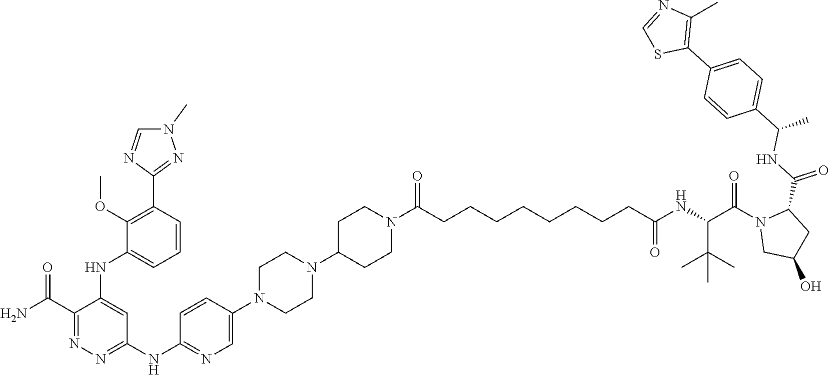 [SMILES: COc1c(Nc2cc(Nc3ccc(N4CCN(C5CCN(C(=O)CCCCCCCCC(=O)N[C@H](C(=O)N6C[C@H](O)C[C@H]6C(=O)N[C@@H](C)c6ccc(-c7scnc7C)cc6)C(C)(C)C)CC5)CC4)cn3)nnc2C(N)=O)cccc1-c1ncn(C)n1] |
| CPD- 189 | 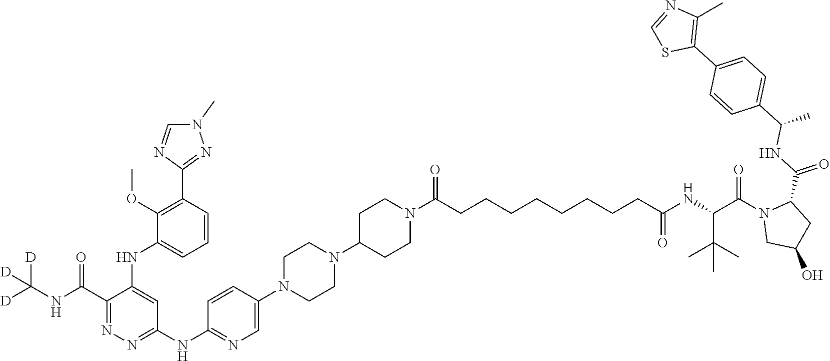 [SMILES: [2H]C([2H])([2H])NC(=O)c1nnc(Nc2ccc(N3CCN(C4CCN(C(=O)CCCCCCCCC(=O)N[C@H](C(=O)N5C[C@H](O)C[C@H]5C(=O)N[C@@H](C)c5ccc(-c6scnc6C)cc5)C(C)(C)C)CC4)CC3)cn2)cc1Nc1cccc(-c2ncn(C)n2)c1OC] |
| CPD- 190 | 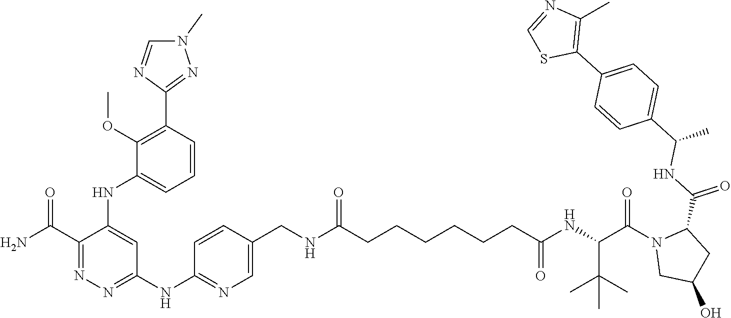 [SMILES: COc1c(Nc2cc(Nc3ccc(CNC(=O)CCCCCCC(=O)N[C@H](C(=O)N4C[C@H](O)C[C@H]4C(=O)N[C@@H](C)c4ccc(-c5scnc5C)cc4)C(C)(C)C)cn3)nnc2C(N)=O)cccc1-c1ncn(C)n1] |
| CPD- 191 | 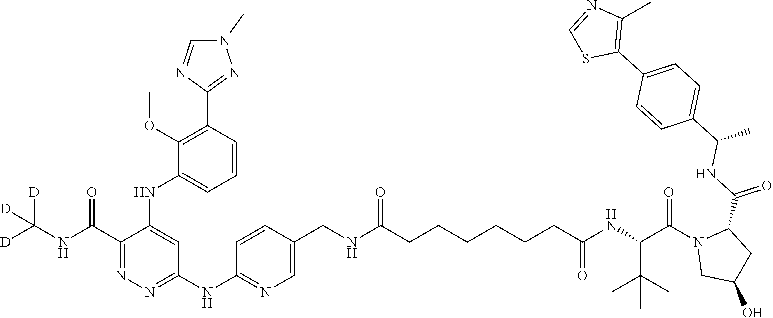 [SMILES: [2H]C([2H])([2H])NC(=O)c1nnc(Nc2ccc(CNC(=O)CCCCCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC] |
| CPD- 192 | 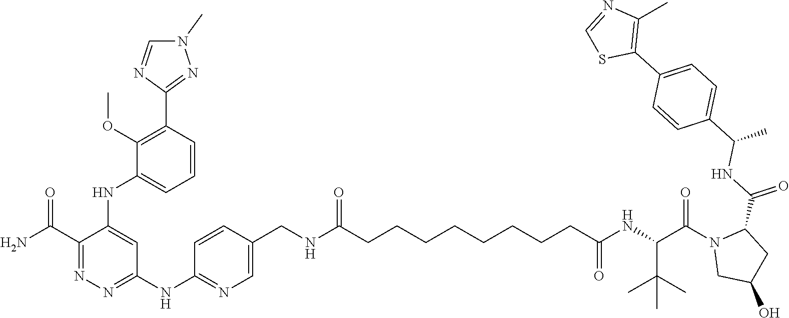 [SMILES: COc1c(Nc2cc(Nc3ccc(CNC(=O)CCCCCCCCC(=O)N[C@H](C(=O)N4C[C@H](O)C[C@H]4C(=O)N[C@@H](C)c4ccc(-c5scnc5C)cc4)C(C)(C)C)cn3)nnc2C(N)=O)cccc1-c1ncn(C)n1] |
| CPD- 193 | 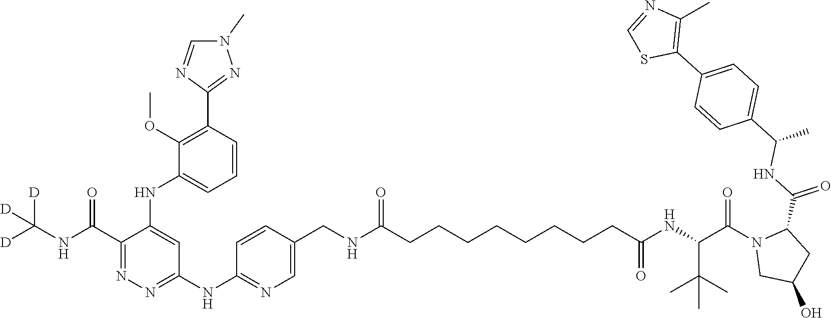 [SMILES: [2H]C([2H])([2H])NC(=O)c1nnc(Nc2ccc(CNC(=O)CCCCCCCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC] |
| CPD- 194 | 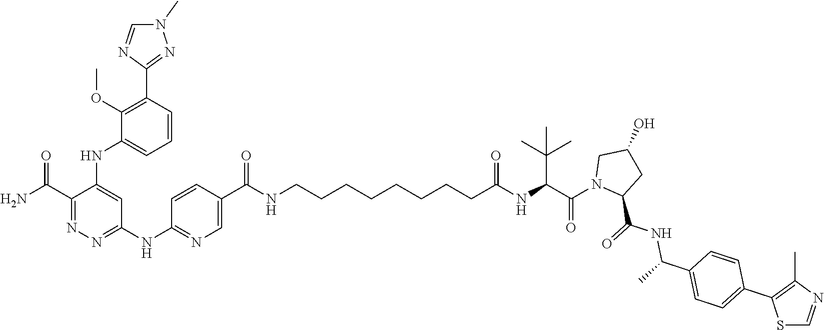 [SMILES: COc1c(Nc2cc(Nc3ccc(C(=O)NCCCCCCCCC(=O)N[C@H](C(=O)N4C[C@H](O)C[C@H]4C(=O)N[C@@H](C)c4ccc(-c5scnc5C)cc4)C(C)(C)C)cn3)nnc2C(N)=O)cccc1-c1ncn(C)n1] |
| CPD- 195 | 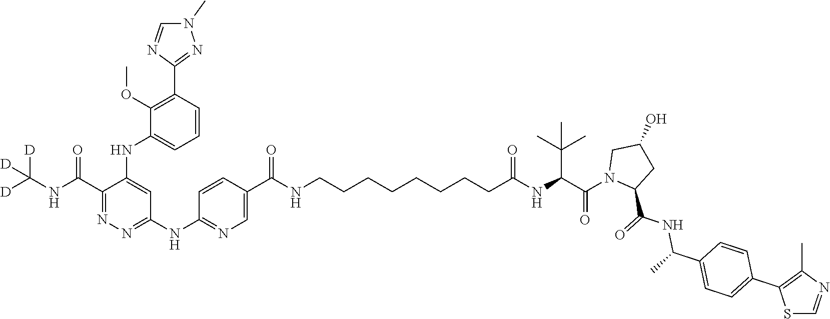 [SMILES: [2H]C([2H])([2H])NC(=O)c1nnc(Nc2ccc(C(=O)NCCCCCCCCC(=O)N[C@H](C(=O)N3C[C@H](O)C[C@H]3C(=O)N[C@@H](C)c3ccc(-c4scnc4C)cc3)C(C)(C)C)cn2)cc1Nc1cccc(-c2ncn(C)n2)c1OC] |
| CPD- 196 | 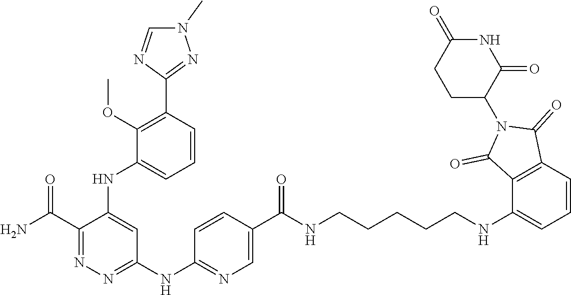 [SMILES: COc1c(Nc2cc(Nc3ccc(C(=O)NCCCCCNc4cccc5c4C(=O)N(C4CCC(=O)NC4=O)C5=O)cn3)nnc2C(N)=O)cccc1-c1ncn(C)n1] |
| CPD- 197 | 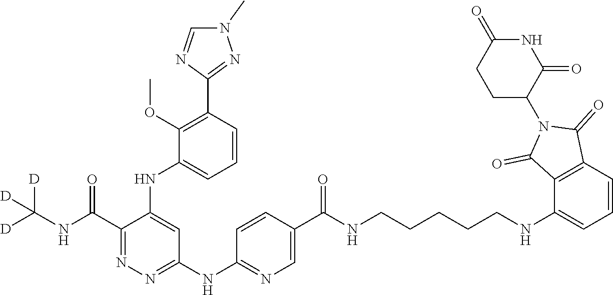 [SMILES: [2H]C([2H])([2H])NC(=O)c1nnc(Nc2ccc(C(=O)NCCCCCNc3cccc4c3C(=O)N(C3CCC(=O)NC3=O)C4=O)cn2)cc1Nc1cccc(-c2ncn(C)n2)c1OC] |
| CPD- 198 | 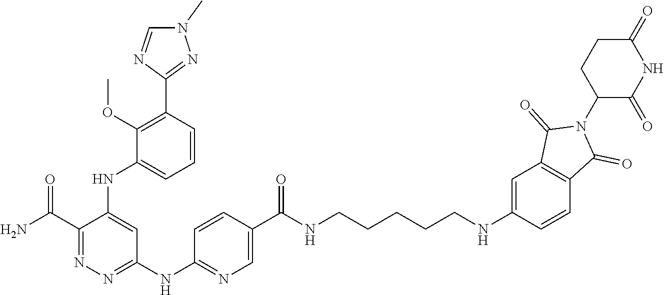 [SMILES: COc1c(Nc2cc(Nc3ccc(C(=O)NCCCCCNc4ccc5c(c4)C(=O)N(C4CCC(=O)NC4=O)C5=O)cn3)nnc2C(N)=O)cccc1-c1ncn(C)n1] |
| CPD- 199 | 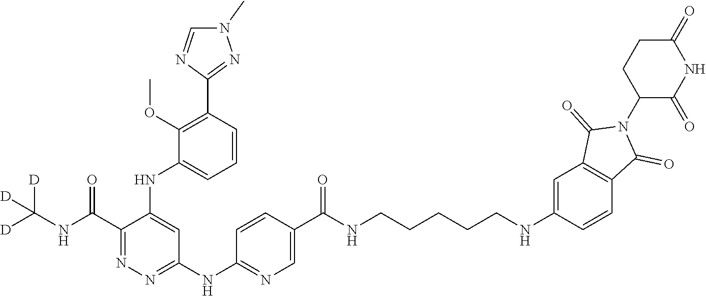 [SMILES: [2H]C([2H])([2H])NC(=O)c1nnc(Nc2ccc(C(=O)NCCCCCNc3ccc4c(c3)C(=O)N(C3CCC(=O)NC3=O)C4=O)cn2)cc1Nc1cccc(-c2ncn(C)n2)c1OC] |
| Cpd. | Chemical Name |
| CPD- | 6-(cyclopropanecarboxamido)-4-((3-(1-(2-(2-((2-(2,6-dioxopiperidin-3-yl)-1,3-dioxoisoindolin-4-yl)amino)acetamido)ethyl)-1H-1,2,4- |
| 001 | triazol-3-yl)-2-methoxyphenyl)amino)-N-methylpyridazine-3-carboxamide |
| CPD- | 6-(cyclopropanecarboxamido)-4-((3-(1-(2-(3-((2-(2,6-dioxopiperidin-3-yl)-1,3-dioxoisoindolin-4-yl)amino)propanamido)ethyl)-1H-1,2,4- |
| 002 | triazol-3-yl)-2-methoxyphenyl)amino)-N-methylpyridazine-3-carboxamide |
| CPD- | 6-(cyclopropanecarboxamido)-4-((3-(1-(2-(4-((2-(2,6-dioxopiperidin-3-yl)-1,3-dioxoisoindolin-4-yl)amino)butanamido)ethyl)-1H-1,2,4- |
| 003 | triazol-3-yl)-2-methoxyphenyl)amino)-N-methylpyridazine-3-carboxamide |
| CPD- | 6-(cyclopropanecarboxamido)-4-((3-(1-(2-(5-((2-(2,6-dioxopiperidin-3-yl)-1,3-dioxoisoindolin-4-yl)amino)pentanamido)ethyl)-1H-1,2,4- |
| 004 | triazol-3-yl)-2-methoxyphenyl)amino)-N-methylpyridazine-3-carboxamide |
| CPD- | 6-(cyclopropanecarboxamido)-4-((3-(1-(2-(6-((2-(2,6-dioxopiperidin-3-yl)-1,3-dioxoisoindolin-4-yl)amino)hexanamido)ethyl)-1H-1,2,4- |
| 005 | triazol-3-yl)-2-methoxyphenyl)amino)-N-methylpyridazine-3-carboxamide |
| CPD- | 6-(cyclopropanecarboxamido)-4-((3-(1-(2-(7-((2-(2,6-dioxopiperidin-3-yl)-1,3-dioxoisoindolin-4-yl)amino)heptanamido)ethyl)-1H-1,2,4- |
| 006 | triazol-3-yl)-2-methoxyphenyl)amino)-N-methylpyridazine-3-carboxamide |
| CPD- | 6-(cyclopropanecarboxamido)-4-((3-(1-(2-(8-((2-(2,6-dioxopiperidin-3-yl)-1,3-dioxoisoindolin-4-yl)amino)octanamido)ethyl)-1H-1,2,4- |
| 007 | triazol-3-yl)-2-methoxyphenyl)amino)-N-methylpyridazine-3-carboxamide |
| CPD- | 6-(cyclopropanecarboxamido)-4-((3-(1-(2-(3-(2-((2-(2,6-dioxopiperidin-3-yl)-1,3-dioxoisoindolin-4-yl)amino)ethoxy)propanamido)ethyl)-1H- |
| 008 | 1,2,4-triazol-3-yl)-2-methoxyphenyl)amino)-N-methylpyridazine-3-carboxamide |
| CPD- | 6-(cyclopropanecarboxamido)-4-((3-(1-(2-(3-(2-(2-((2-(2,6-dioxopiperidin-3-yl)-1,3-dioxoisoindolin-4-yl)amino)ethoxy)ethoxy)propanamido) |
| 009 | ethyl)-1H-1,2,4-triazol-3-yl)-2-methoxyphenyl)amino)-N-methylpyridazine-3-carboxamide |
| CPD- | 6-(cyclopropanecarboxamido)-4-((3-(1-(1-((2-(2,6-dioxopiperidin-3-yl)-1,3-dioxoisoindolin-4-yl)amino)-12-oxo-3,6,9-trioxa-13- |
| 010 | azapentadecan-15-yl)-1H-1,2,4-triazol-3-yl)-2-methoxyphenyl)amino)-N-methylpyridazine-3-carboxamide |
| CPD- | 6-(cyclopropanecarboxamido)-4-((3-(1-(1-((2-(2,6-dioxopiperidin-3-yl)-1,3-dioxoisoindolin-4-yl)amino)-15-oxo-3,6,9,12-tetraoxa-16- |
| 011 | azaoctadecan-18-yl)-1H-1,2,4-triazol-3-yl)-2-methoxyphenyl)amino)-N-methylpyridazine-3-carboxamide |
| CPD- | 6-(cyclopropanecarboxamido)-4-((3-(1-(1-((2-(2,6-dioxopiperidin-3-yl)-1,3-dioxoisoindolin-4-yl)amino)-18-oxo-3,6,9,12,15-pentaoxa-19- |
| 012 | azahenicosan-21-yl)-1H-1,2,4-triazol-3-yl)-2-methoxyphenyl)amino)-N-methylpyridazine-3-carboxamide |
| CPD- | 6-(cyclopropanecarboxamido)-4-((3-(1-(2-(2-((2-(2,6-dioxopiperidin-3-yl)-1,3-dioxoisoindolin-5-yl)amino)acetamido)ethyl)-1H-1,2,4- |
| 013 | triazol-3-yl)-2-methoxyphenyl)amino)-N-methylpyridazine-3-carboxamide |
| CPD- | 6-(cyclopropanecarboxamido)-4-((3-(1-(2-(3-((2-(2,6-dioxopiperidin-3-yl)-1,3-dioxoisoindolin-5-yl)amino)propanamido)ethyl)-1H-1,2,4- |
| 014 | triazol-3-yl)-2-methoxyphenyl)amino)-N-methylpyridazine-3-carboxamide |
| CPD- | 6-(cyclopropanecarboxamido)-4-((3-(1-(2-(4-((2-(2,6-dioxopiperidin-3-yl)-1,3-dioxoisoindolin-5-yl)amino)butanamido)ethyl)-1H-1,2,4- |
| 015 | triazol-3-yl)-2-methoxyphenyl)amino)-N-methylpyridazine-3-carboxamide |
| CPD- | 6-(cyclopropanecarboxamido)-4-((3-(1-(2-(5-((2-(2,6-dioxopiperidin-3-yl)-1,3-dioxoisoindolin-5-yl)amino)pentanamido)ethyl)-1H-1,2,4- |
| 016 | triazol-3-yl)-2-methoxyphenyl)amino)-N-methylpyridazine-3-carboxamide |
| CPD- | 6-(cyclopropanecarboxamido)-4-((3-(1-(2-(7-((2-(2,6-dioxopiperidin-3-yl)-1,3-dioxoisoindolin-5-yl)amino)heptanamido)ethyl)-1H-1,2,4- |
| 017 | triazol-3-yl)-2-methoxyphenyl)amino)-N-methylpyridazine-3-carboxamide |
| CPD- | 6-(cyclopropanecarboxamido)-4-((3-(1-(2-(3-(2-((2-(2,6-dioxopiperidin-3-yl)-1,3-dioxoisoindolin-5-yl)amino)ethoxy)propanamido)ethyl)- |
| 018 | 1H-1,2,4-triazol-3-yl)-2-methoxyphenyl)amino)-N-methylpyridazine-3-carboxamide |
| CPD- | 6-(cyclopropanecarboxamido)-4-((3-(1-(2-(3-(2-(2-((2-(2,6-dioxopiperidin-3-yl)-1,3-dioxoisoindolin-5-yl)amino)ethoxy)ethoxy) |
| 019 | propanamido)ethyl)-1H-1,2,4-triazol-3-yl)-2-methoxyphenyl)amino)-N-methylpyridazine-3-carboxamide |
| CPD- | 6-(cyclopropanecarboxamido)-4-((3-(1-(1-((2-(2,6-dioxopiperidin-3-yl)-1,3-dioxoisoindolin-5-yl)amino)-12-oxo-3,6,9-trioxa-13- |
| 020 | azapentadecan-15-yl)-1H-1,2,4-triazol-3-yl)-2-methoxyphenyl)amino)-N-methylpyridazine-3-carboxamide |
| CPD- | 6-(cyclopropanecarboxamido)-4-((3-(1-(1-((2-(2,6-dioxopiperidin-3-yl)-1,3-dioxoisoindolin-5-yl)amino)-15-oxo-3,6,9,12-tetraoxa-16- |
| 021 | azaoctadecan-18-yl)-1H-1,2,4-triazol-3-yl)-2-methoxyphenyl)amino)-N-methylpyridazine-3-carboxamide |
| CPD- | 6-(cyclopropanecarboxamido)-4-((3-(1-(1-((2-(2,6-dioxopiperidin-3-yl)-1,3-dioxoisoindolin-5-yl)amino)-18-oxo-3,6,9,12,15-pentaoxa-19- |
| 022 | azahenicosan-21-yl)-1H-1,2,4-triazol-3-yl)-2-methoxyphenyl)amino)-N-methylpyridazine-3-carboxamide |
| CPD- | 6-(cyclopropanecarboxamido)-4-((3-(1-(2-(6-((2-(2,6-dioxopiperidin-3-yl)-1,3-dioxoisoindolin-5-yl)amino)hexanamido)ethyl)-1H-1,2,4- |
| 023 | triazol-3-yl)-2-methoxyphenyl)amino)-N-methylpyridazine-3-carboxamide |
| CPD- | 6-(cyclopropanecarboxamido)-4-((3-(1-(2-(8-((2-(2,6-dioxopiperidin-3-yl)-1,3-dioxoisoindolin-5-yl)amino)octanamido)ethyl)-1H-1,2,4- |
| 024 | triazol-3-yl)-2-methoxyphenyl)amino)-N-methylpyridazine-3-carboxamide |
| CPD- | 6-(2-(2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)- |
| 025 | 2-oxoethoxy)acetamido)acetamido)-4-((2-methoxy-3-(1-methyl-1H-1,2,4-triazol-3-yl)phenyl)amino)-N-methylpyridazine-3-carboxamide |
| CPD- | 6-(2-(3-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)- |
| 026 | 3-oxopropoxy)propanamido)acetamido)-4-((2-methoxy-3-(1-methyl-1,2,4-triazol-3-yl)phenyl)amino)-N-methylpyridazine-3-carboxamide |
| CPD- | 6-((S)-13-((2S,4R)-4-hydroxy-2-((4-(4-methylthiazol-5-yl)benzyl)carbamoyl)pyrrolidine-1-carbonyl)-14,14-dimethyl-4,11-dioxo-6,9-dioxa- |
| 027 | 3,12-diazapentadecanamido)-4-((2-methoxy-3-(1-methyl-1H-1,2,4-triazol-3-yl)phenyl)amino)-N-methylpyridazine-3-carboxamide |
| CPD- | 6-((S)-15-((2S,4R)-4-hydroxy-2-((4-(4-methylthiazol-5-yl)benzyl)carbamoyl)pyrrolidine-1-carbonyl)-16,16-dimethyl-4,13-dioxo-7,19-dioxa- |
| 028 | 3,14-diazaheptadecanamido)-4-((2-methoxy-3-(1-methyl-1H-1,2,4-triazol-3-yl)phenyl)amino)-N-methylpyridazine-3-carboxamide |
| CPD- | 6-((S)-16-((2S,4R)-4-hydroxy-2-((4-(4-methylthiazol-5-yl)benzyl)carbamoyl)pyrrolidine-1-carbonyl)-17,17-dimethyl-4,14-dioxo- |
| 029 | 6,9,12-trioxa-3,15-diazaoctadecanamido)-4-((2-methoxy-3-(1-methyl-1H-1,2,4-triazol-3-yl)phenyl)amino)-N-methylpyridazine-3-carboxamide |
| CPD- | 6-((S)-18-((2S,4R)-4-hydroxy-2-((4-(4-methylthiazol-5-yl)benzyl)carbamoyl)pyrrolidine-1-carbonyl)-19,19-dimethyl-4,16-dioxo-7,10,13- |
| 030 | trioxa-3,17-diazaicosanamido)-4-((2-methoxy-3-(1-methyl-1H-1,2,4-triazol-3-yl)phenyl)amino)-N-methylpyridazine-3-carboxamide |
| CPD- | N1-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N16-(2- |
| 031 | ((5-((2-methoxy-3-(1-methyl-1H-1,2,4-triazol-3-yl)phenyl)amino)-6-(methylcarbamoyl)pyridazin-3-yl)amino)-2-oxoethyl)-4,7,10,13- |
| | tetraoxahexadecanediamide |
| CPD- | N1-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N17-(2- |
| 032 | ((5-((2-methoxy-3-(1-methyl-1H-1,2,4-triazol-3-yl)phenyl)amino)-6-(methylcarbamoyl)pyridazin-3-yl)amino)-2-oxoethyl)-3,6,9,12,15- |
| | pentaoxaheptadecanediamide |
| CPD- | N1-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N19-(2- |
| 033 | ((5-((2-methoxy-3-(1-methyl-1H-1,2,4-triazol-3-yl)phenyl)amino)-6-(methylcarbamoyl)pyridazin-3-yl)amino)-2-oxoethyl)-4,7,10,13,16- |
| | pentaoxanonadecanediamide |
| CPD- | N1-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N4-(2- |
| 034 | ((5-((2-methoxy-3-(1-methyl-1H-1,2,4-triazol-3-yl)phenyl)amino)-6-(methylcarbamoyl)pyridazin-3-yl)amino)-2-oxoethyl)succinamide |
| CPD- | N1-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N5-(2- |
| 035 | ((5-((2-methoxy-3-(1-methyl-1H-1,2,4-triazol-3-yl)phenyl)amino)-6-(methylcarbamoyl)pyridazin-3-yl)amino)-2-oxoethyl)glutaramide |
| CPD- | N1-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N6-(2- |
| 036 | ((5-((2-methoxy-3-(1-methyl-1H-1,2,4-triazol-3-yl)phenyl)amino)-6-(methylcarbamoyl)pyridazin-3-yl)amino)-2-oxoethyl)adipamide |
| CPD- | N1-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N7-(2- |
| 037 | ((5-((2-methoxy-3-(1-methyl-1H-1,2,4-triazol-3-yl)phenyl)amino)-6-(methylcarbamoyl)pyridazin-3-yl)amino)-2-oxoethyl)heptanediamide |
| CPD- | N1-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N8-(2- |
| 038 | ((5-((2-methoxy-3-(1-methyl-1H-1,2,4-triazol-3-yl)phenyl)amino)-6-(methylcarbamoyl)pyridazin-3-yl)amino)-2-oxoethyl)octanediamide |
| CPD- | N1-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N9-(2- |
| 039 | ((5-((2-methoxy-3-(1-methyl-1H-1,2,4-triazol-3-yl)phenyl)amino)-6-(methylcarbamoyl)pyridazin-3-yl)amino)-2-oxoethyl)nonanediamide |
| CPD- | N1-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N10-(2- |
| 040 | ((5-((2-methoxy-3-(1-methyl-1H-1,2,4-triazol-3-yl)phenyl)amino)-6-(methylcarbamoyl)pyridazin-3-yl)amino)-2-oxoethyl)decanediamide |
| CPD- | N1-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N11-(2- |
| 041 | ((5-((2-methoxy-3-(1-methyl-1H-1,2,4-triazol-3-yl)phenyl)amino)-6-(methylcarbamoyl)pyridazin-3-yl)amino)-2-oxoethyl)undecanediamide |
| CPD- | 6-(2-(2-((2-(2,6-dioxopiperidin-3-yl)-1,3-dioxoisoindolin-4-yl)amino)acetamido)acetamido)-4-((2-methoxy-3-(1-methyl-1H-1,2,4- |
| 042 | triazol-3-yl)phenyl)amino)-N-methylpyridazine-3-carboxamide |
| CPD- | 6-(2-(3-((2-(2,6-dioxopiperidin-3-yl)-1,3-dioxoisoindolin-4-yl)amino)propanamido)acetamido)-4-((2-methoxy-3-(1-methyl-1H- |
| 043 | 1,2,4-triazol-3-yl)phenyl)amino)-N-methylpyridazine-3-carboxamide |
| CPD- | 6-(2-(4-((2-(2,6-dioxopiperidin-3-yl)-1,3-dioxoisoindolin-4-yl)amino)butanamido)acetamido)-4-((2-methoxy-3-(1-methyl-1H- |
| 044 | 3-yl)phenyl)amino)-N-methylpyridazine-3-carboxamide |
| CPD- | 6-(2-(5-((2-(2,6-dioxopiperidin-3-yl)-1,3-dioxoisoindolin-4-yl)amino)pentanamido)acetamido)-4-((2-methoxy-3-(1-methyl-1H- |
| 045 | 1,2,4-triazol-3-yl)phenyl)amino)-N-methylpyridazine-3-carboxamide |
| CPD- | 6-(2-(6-((2-(2,6-dioxopiperidin-3-yl)-1,3-dioxoisoindolin-4-yl)amino)hexanamido)acetamido)-4-((2-methoxy-3-(1-methyl-1H- |
| 046 | 1,2,4-triazol-3-yl)phenyl)amino)-N-methylpyridazine-3-carboxamide |
| CPD- | 6-(2-(7-((2-(2,6-dioxopiperidin-3-yl)-1,3-dioxoisoindolin-4-yl)amino)heptanamido)acetamido)-4-((2-methoxy-3-(1-methyl-1H- |
| 047 | 1,2,4-triazol-3-yl)phenyl)amino)-N-methylpyridazine-3-carboxamide |
| CPD- | 6-(2-(8-((2-(2,6-dioxopiperidin-3-yl)-1,3-dioxoisoindolin-4-yl)amino)octanamido)acetamido)-4-((2-methoxy-3-(1-methyl-1H- |
| 048 | 1,2,4-triazol-3-yl)phenyl)amino)-N-methylpyridazine-3-carboxamide |
| CPD- | 6-(2-(3-(2-((2-(2,6-dioxopiperidin-3-yl)-1,3-dioxoisoindolin-4-yl)amino)ethoxy)propanamido)acetamido)-4-((2-methoxy-3-(1-methyl-1H- |
| 049 | 1,2,4-triazol-3-yl)phenyl)amino)-N-methylpyridazine-3-carboxamide |
| CPD- | 6-(2-(3-(2-(2-((2-(2,6-dioxopiperidin-3-yl)-1,3-dioxoisoindolin-4-yl)amino)ethoxy)ethoxy)propanamido)acetamido)-4- |
| 050 | ((2-methoxy-3-(1-methyl-1H-1,2,4-triazol-3-yl)phenyl)amino)-N-methylpyridazine-3-carboxamide |
| CPD- | 6-(1-((2-(2,6-dioxopiperidin-3-yl)-1,3-dioxoisoindolin-4-yl)amino)-12-oxo-3,6,9-trioxa-13-azapentadecan-15-amido)-4-((2-methoxy-3-(1- |
| 051 | methyl-1H-1,2,4-triazol-3-yl)phenyl)amino)-N-methylpyridazine-3-carboxamide |
| CPD- | 6-(1-((2-(2,6-dioxopiperidin-3-yl)-1,3-dioxoisoindolin-4-yl)amino)-15-oxo-3,6,9,12-tetraoxa-16-azaoctadecan-18-amido)-4-((2-methoxy-3-(1- |
| 052 | methyl-1H-1,2,4-triazol-3-yl)phenyl)amino)-N-methylpyridazine-3-carboxamide |
| CPD- | 6-(1-((2-(2,6-dioxopiperidin-3-yl)-1,3-dioxoisoindolin-4-yl)amino)-18-oxo-3,6,9,12,15-pentaoxa-19-azahenicosan-21-amido)-4-((2-methoxy- |
| 053 | 3-(1-methyl-1H-1,2,4-triazol-3-yl)phenyl)amino)-N-methylpyridazine-3-carboxamide |
| CPD- | 6-(2-(2-((2-(2,6-dioxopiperidin-3-yl)-1,3-dioxoisoindolin-5-yl)amino)acetamido)acetamido)-4-((2-methoxy-3-(1-methyl-1H-1,2,4-triazol-3-yl) |
| 054 | phenyl)amino)-N-methylpyridazine-3-carboxamide |
| CPD- | 6-(2-(3-((2-(2,6-dioxopiperidin-3-yl)-1,3-dioxoisoindolin-5-yl)amino)propanamido)acetamido)-4-((2-methoxy-3-(1-methyl-1H-1,2,4-triazol- |
| 055 | 3-yl)phenyl)amino)-N-methylpyridazine-3-carboxamide |
| CPD- | 6-(2-(4-((2-(2,6-dioxopiperidin-3-yl)-1,3-dioxoisoindolin-5-yl)amino)butanamido)acetamido)-4-((2-methoxy-3-(1-methyl-1H-1,2,4-triazol- |
| 056 | 3-yl)phenyl)amino)-N-methylpyridazine-3-carboxamide |
| CPD- | 6-(2-(5-((2-(2,6-dioxopiperidin-3-yl)-1,3-dioxoisoindolin-5-yl)amino)pentanamido)acetamido)-4-((2-methoxy-3-(1-methyl-1H-1,2,4-triazol- |
| 057 | 3-yl)phenyl)amino)-N-methylpyridazine-3-carboxamide |
| CPD- | 6-(2-(6-((2-(2,6-dioxopiperidin-3-yl)-1,3-dioxoisoindolin-5-yl)amino)hexanamido)acetamido)-4-((2-methoxy-3-(1-methyl-1H-1,2,4-triazol- |
| 058 | 3-yl)phenyl)amino)-N-methylpyridazine-3-carboxamide |
| CPD- | 6-(2-(7-((2-(2,6-dioxopiperidin-3-yl)-1,3-dioxoisoindolin-5-yl)amino)heptanamido)acetamido)-4-((2-methoxy-3-(1-methyl-1H-1,2,4-triazol- |
| 059 | 3-yl)phenyl)amino)-N-methylpyridazine-3-carboxamide |
| CPD- | 6-(2-(8-((2-(2,6-dioxopiperidin-3-yl)-1,3-dioxoisoindolin-5-yl)amino)octanamido)acetamido)-4-((2-methoxy-3-(1-methyl-1H-1,2,4-triazol- |
| 060 | 3-yl)phenyl)amino)-N-methylpyridazine-3-carboxamide |
| CPD- | 6-(2-(3-(2-((2-(2,6-dioxopiperidin-3-yl)-1,3-dioxoisoindolin-5-yl)amino)ethoxy)propanamido)acetamido)-4-((2-methoxy-3-(1-methyl-1H- |
| 061 | 1,2,4-triazol-3-yl)phenyl)amino)-N-methylpyridazine-3-carboxamide |
| CPD- | 6-(2-(3-(2-(2-((2-(2,6-dioxopiperidin-3-yl)-1,3-dioxoisoindolin-5-yl)amino)ethoxy)ethoxy)propanamido)acetamido)-4-((2-methoxy-3- |
| 062 | (1-methyl-1H-1,2,4-triazol-3-yl)phenyl)amino)-N-methylpyridazine-3-carboxamide |
| CPD- | 6-(1-((2-(2,6-dioxopiperidin-3-yl)-1,3-dioxoisoindolin-5-yl)amino)-12-oxo-3,6,9-trioxa-13-azapentadecan-15-amido)-4-((2-methoxy-3- |
| 063 | (1-methyl-1H-1,2,4-triazol-3-yl)phenyl)amino)-N-methylpyridazine-3-carboxamide |
| CPD- | 6-(1-((2-(2,6-dioxopiperidin-3-yl)-1,3-dioxoisoindolin-5-yl)amino)-15-oxo-3,6,9,12-tetraoxa-16-azaoctadecan-18-amido)-4-((2-methoxy-3-(1- |
| 064 | methyl-1H-1,2,4-triazol-3-yl)phenyl)amino)-N-methylpyridazine-3-carboxamide |
| CPD- | 6-(1-((2-(2,6-dioxopiperidin-3-yl)-1,3-dioxoisoindolin-5-yl)amino)-18-oxo-3,6,9,12,15-pentaoxa-19-azahenicosan-21-amido)-4-((2-methoxy- |
| 065 | 3-(1-methyl-1H-1,2,4-triazol-3-yl)phenyl)amino)-N-methylpyridazine-3-carboxamide |
| CPD- | 6-(cyclopropanecarboxamido)-4-((3-(1-(2-(2-(2-(((S)-1-((2S,4R)-4-hydroxy-2-((4-(4-methylthiazol-5-yl)benzyl)carbamoyl)pyrrolidin-1-yl)- |
| 066 | 3,3-dimethyl-1-oxobutan-2-yl)amino)-2-oxoethoxy)acetamido)ethyl)-1H-1,2,4-triazol-3-yl)-2-methoxyphenyl)amino)-N- |
| | methylpyridazine-3-carboxamide |
| CPD- | 6-(cyclopropanecarboxamido)-4-((3-(1-(2-(3-(3-(((S)-1-((2S,4R)-4-hydroxy-2-((4-(4-methylthiazol-5-yl)benzyl)carbamoyl)pyrrolidin-1-yl)- |
| 067 | 3,3-dimethyl-1-oxobutan-2-yl)amino)-3-oxopropoxy)propanamido)ethyl)-1H-1,2,4-triazol-3-yl)-2-methoxyphenyl)amino)-N- |
| | methylpyridazine-3-carboxamide |
| CPD- | 6-(cyclopropanecarboxamido)-4-((3-(1-((S)-13-((2S,4R)-4-hydroxy-2-((4-(4-methylthiazol-5-yl)benzyl)carbamoyl)pyrrolidine-1-carbonyl)- |
| 068 | 14,14-dimethyl-4,11-dioxo-6,9-dioxa-3,12-diazapentadecyl)-1H-1,2,4-triazol-3-yl)-2-methoxyphenyl)amino)-N-methylpyridazine- |
| | 3-carboxamide |
| CPD- | 6-(cyclopropanecarboxamido)-4-((3-(1-((S)-15-((2S,4R)-4-hydroxy-2-((4-(4-methylthiazol-5-yl)benzyl)carbamoyl)pyrrolidine-1-carbonyl)- |
| 069 | 16,16-dimethyl-4,13-dioxo-7,10-dioxa-3,14-diazaheptadecyl)-1H-1,2,4-triazol-3-yl)-2-methoxyphenyl)amino)-N-methylpyridazine- |
| | 3-carboxamide |
| CPD- | 6-(cyclopropanecarboxamido)-4-((3-(1-((S)-16-((2S,4R)-4-hydroxy-2-((4-(4-methylthiazol-5-yl)benzyl)carbamoyl)pyrrolidine-1-carbonyl)- |
| 070 | 17,17-dimethyl-4,14-dioxo-6,9,12-trioxa-3,15-diazaoctadecyl)-1H-1,2,4-triazol-3-yl)-2-methoxyphenyl)amino)-N-methylpyridazine- |
| | 3-carboxamide |
| CPD- | 6-(cyclopropanecarboxamido)-4-((3-(1-((S)-18-((2S,4R)-4-hydroxy-2-((4-(4-methylthiazol-5-yl)benzyl)carbamoyl)pyrrolidine-1-carbonyl)- |
| 071 | 19,19-dimethyl-4,16-dioxo-7,10,13-trioxa-3,17-diazaicosyl)-1H-1,2,4-triazol-3-yl)-2-methoxyphenyl)amino)-N-methylpyridazine- |
| | 3-carboxamide |
| CPD- | N1-(2-(3-(3-((6-(cyclopropanecarboxamido)-3-(methylcarbamoyl)pyridazin-4-yl)amino)-2-methoxyphenyl)-1H-1,2,4-triazol-1-yl)ethyl)-N16- |
| 072 | ((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-4,7,10,13- |
| | tetraoxahexadecanediamide |
| CPD- | N1-(2-(3-(3-((6-(cyclopropanecarboxamido)-3-(methylcarbamoyl)pyridazin-4-yl)amino)-2-methoxyphenyl)-1H-1,2,4-triazol-1-yl)ethyl)-N17- |
| 073 | ((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-3,6,9,12,15- |
| | pentaoxaheptadecanediamide |
| CPD- | N1-(2-(3-(3-((6-(cyclopropanecarboxamido)-3-(methylcarbamoyl)pyridazin-4-yl)amino)-2-methoxyphenyl)-1H-1,2,4-triazol-1-yl)ethyl)-N19- |
| 074 | ((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-4,7,10,13,16- |
| | pentaoxanonadecanediamide |
| CPD- | N1-(2-(3-(3-((6-(cyclopropanecarboxamido)-3-(methylcarbamoyl)pyridazin-4-yl)amino)-2-methoxyphenyl)-1H-1,2,4-triazol-1-yl)ethyl)-N4- |
| 075 | ((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)succinamide |
| CPD- | N1-(2-(3-(3-((6-(cyclopropanecarboxamido)-3-(methylcarbamoyl)pyridazin-4-yl)amino)-2-methoxyphenyl)-1H-1,2,4-triazol-1-yl)ethyl)-N5- |
| 076 | ((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)glutaramide |
| CPD- | N1-(2-(3-(3-((6-(cyclopropanecarboxamido)-3-(methylcarbamoyl)pyridazin-4-yl)amino)-2-methoxyphenyl)-1H-1,2,4-triazol-1-yl)ethyl)-N6- |
| 077 | ((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)adipamide |
| CPD- | N1-(2-(3-(3-((6-(cyclopropanecarboxamido)-3-(methylcarbamoyl)pyridazin-4-yl)amino)-2-methoxyphenyl)-1H-1,2,4-triazol-1-yl)ethyl)-N7- |
| 078 | ((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)heptanediamide |
| CPD- | N1-(2-(3-(3-((6-(cyclopropanecarboxamido)-3-(methylcarbamoyl)pyridazin-4-yl)amino)-2-methoxyphenyl)-1H-1,2,4-triazol-1-yl)ethyl)-N8- |
| 079 | ((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)octanediamide |
| CPD- | N1-(2-(3-(3-((6-(cyclopropanecarboxamido)-3-(methylcarbamoyl)pyridazin-4-yl)amino)-2-methoxyphenyl)-1H-1,2,4-triazol-1-yl)ethyl)-N9- |
| 080 | ((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)nonanediamide |
| CPD- | N1-(2-(3-(3-((6-(cyclopropanecarboxamido)-3-(methylcarbamoyl)pyridazin-4-yl)amino)-2-methoxyphenyl)-1H-1,2,4-triazol-1-yl)ethyl)-N10- |
| 081 | ((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)decanediamide |
| CPD- | N1-(2-(3-(3-((6-(cyclopropanecarboxamido)-3-(methylcarbamoyl)pyridazin-4-yl)amino)-2-methoxyphenyl)-1H-1,2,4-triazol-1-yl)ethyl)-N11- |
| 082 | ((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)undecanediamide |
| CPD- | N1-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2- |
| 083 | yl)-N8-(2-((5-((2-methoxy-3-(1-methyl-1H-1,2,4-triazol-3-yl)phenyl)amino)-6-(methylcarbamoyl)pyridazin-3-yl)amino)-2- |
| | oxoethyl)octanediamide |
| CPD- | N1-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2- |
| 084 | yl)-N9-(2-((5-((2-methoxy-3-(1-methyl-1H-1,2,4-triazol-3-yl)phenyl)amino)-6-(methylcarbamoyl)pyridazin-3-yl)amino)-2- |
| | oxoethyl)nonanediamide |
| CPD- | N1-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)- |
| 085 | N10-(2-((5-((2-methoxy-3-(1-methyl-1H-1,2,4-triazol-3-yl)phenyl)amino)-6-(methylcarbamoyl)pyridazin-3-yl)amino)-2- |
| | oxoethyl)decanediamide |
| CPD- | 6-(2-(2-((S)-3-((2S,4R)-1-((S)-2-(1-fluorocyclopropane-1-carboxamido)-3,3-dimethylbutanoyl)-4-hydroxypyrrolidine-2-carboxamido)-3- |
| 086 | (4-(4-methylthiazol-5-yl)phenyl)propanamido)acetamido)acetamido)-4-((2-methoxy-3-(1-methyl-1H-1,2,4-triazol-3-yl)phenyl)amino)-N- |
| | methylpyridazine-3-carboxamide |
| CPD- | 6-(2-(3-((S)-3-((2S,4R)-1-((S)-2-(1-fluorocyclopropane-1-carboxamido)-3,3-dimethylbutanoyl)-4-hydroxypyrrolidine-2-carboxamido)-3- |
| 087 | (4-(4-methylthiazol-5-yl)phenyl)propanamido)propanamido)acetamido)-4-((2-methoxy-3-(1-methyl-1H-1,2,4-triazol-3-yl)phenyl)amino)-N- |
| | methylpyridazine-3-carboxamide |
| CPD- | 6-(2-(4-((S)-3-((2S,4R)-1-((S)-2-(1-fluorocyclopropane-1-carboxamido)-3,3-dimethylbutanoyl)-4-hydroxypyrrolidine-2-carboxamido)-3- |
| 088 | (4-(4-methylthiazol-5-yl)phenyl)propanamido)butanamido)acetamido)-4-((2-methoxy-3-(1-methyl-1H-1,2,4-triazol-3-yl)phenyl)amino)-N- |
| | methylpyridazine-3-carboxamide |
| CPD- | 6-(2-(5-((S)-3-((2S,4R)-1-((S)-2-(1-fluorocyclopropane-1-carboxamido)-3,3-dimethylbutanoyl)-4-hydroxypyrrolidine-2-carboxamido)-3- |
| 089 | (4-(4-methylthiazol-5-yl)phenyl)propanamido)pentanamido)acetamido)-4-((2-methoxy-3-(1-methyl-1H-1,2,4-triazol-3-yl)phenyl)amino)-N- |
| | methylpyridazine-3-carboxamide |
| CPD- | 6-(2-(6-((S)-3-((2S,4R)-1-((S)-2-(1-fluorocyclopropane-1-carboxamido)-3,3-dimethylbutanoyl)-4-hydroxypyrrolidine-2-carboxamido)-3- |
| 090 | (4-(4-methylthiazol-5-yl)phenyl)propanamido)hexanamido)acetamido)-4-((2-methoxy-3-(1-methyl-1H-1,2,4-triazol-3-yl)phenyl)amino)-N- |
| | methylpyridazine-3-carboxamide |
| CPD- | 6-(2-(7-((S)-3-((2S,4R)-1-((S)-2-(1-fluorocyclopropane-1-carboxamido)-3,3-dimethylbutanoyl)-4-hydroxypyrrolidine-2-carboxamido)-3- |
| 091 | (4-(4-methylthiazol-5-yl)phenyl)propanamido)heptanamido)acetamido)-4-((2-methoxy-3-(1-methyl-1H-1,2,4-triazol-3-yl)phenyl)amino)-N- |
| | methylpyridazine-3-carboxamide |
| CPD- | 6-(2-(8-((S)-3-((2S,4R)-1-((S)-2-(1-fluorocyclopropane-1-carboxamido)-3,3-dimethylbutanoyl)-4-hydroxypyrrolidine-2-carboxamido)-3-(4-(4- |
| 092 | methylthiazol-5-yl)phenyl)propanamido)octanamido)acetamido)-4-((2-methoxy-3-(1-methyl-1H-1,2,4-triazol-3-yl)phenyl)amino)-N- |
| | methylpyridazine-3-carboxamide |
| CPD- | 6-(2-(9-((S)-3-((2S,4R)-1-((S)-2-(1-fluorocyclopropane-1-carboxamido)-3,3-dimethylbutanoyl)-4-hydroxypyrrolidine-2-carboxamido)-3- |
| 093 | (4-(4-methylthiazol-5-yl)phenyl)propanamido)nonanamido)acetamido)-4-((2-methoxy-3-(1-methyl-1H-1,2,4-triazol-3-yl)phenyl)amino)-N- |
| | methylpyridazine-3-carboxamide |
| CPD- | 6-(2-(10-((S)-3-((2S,4R)-1-((S)-2-(1-fluorocyclopropane-1-carboxamido)-3,3-dimethylbutanoyl)-4-hydroxypyrrolidine-2-carboxamido)-3-(4- |
| 094 | (4-methylthiazol-5-yl)phenyl)propanamido)decanamido)acetamido)-4-((2-methoxy-3-(1-methyl-1H-1,2,4-triazol-3-yl)phenyl)amino)-N- |
| | methylpyridazine-3-carboxamide |
| CPD- | 6-(2-(11-((S)-3-((2S,4R)-1-((S)-2-(1-fluorocyclopropane-1-carboxamido)-3,3-dimethylbutanoyl)-4-hydroxypyrrolidine-2-carboxamido)-3-(4- |
| 095 | (4-methylthiazol-5-yl)phenyl)propanamido)undecanamido)acetamido)-4-((2-methoxy-3-(1-methyl-1H-1,2,4-triazol-3-yl)phenyl)amino)-N- |
| | methylpyridazine-3-carboxamide |
| CPD- | 6-((5-(4-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)- |
| 096 | 4-oxobutanoyl)piperazin-1-yl)pyridin-2-yl)amino)-4-((2-methoxy-3-(1-methyl-1H-1,2,4-triazol-3-yl)phenyl)amino)-N-methylpyridazine- |
| | 3-carboxamide |
| CPD- | 6-((5-(4-(5-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)- |
| 097 | 5-oxopentanoyl)piperazin-1-yl)pyridin-2-yl)amino)-4-((2-methoxy-3-(1-methyl-1H-1,2,4-triazol-3-yl)phenyl)amino)-N-methylpyridazine-3- |
| | carboxamide |
| CPD- | 6-((5-(4-(6-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)- |
| 098 | 6-oxohexanoyl)piperazin-1-yl)pyridin-2-yl)amino)-4-((2-methoxy-3-(1-methyl-1H-1,2,4-triazol-3-yl)phenyl)amino)-N-methylpyridazine-3- |
| | carboxamide |
| CPD- | 6-((5-(4-(7-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)- |
| 099 | 7-oxoheptanoyl)piperazin-1-yl)pyridin-2-yl)amino)-4-((2-methoxy-3-(1-methyl-1H-1,2,4-triazol-3-yl)phenyl)amino)-N-methylpyridazine-3- |
| | carboxamide |
| CPD- | 6-((5-(4-(8-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)- |
| 100 | 8-oxooctanoyl)piperazin-1-yl)pyridin-2-yl)amino)-4-((2-methoxy-3-(1-methyl-1H-1,2,4-triazol-3-yl)phenyl)amino)-N-methylpyridazine-3- |
| | carboxamide |
| CPD- | 6-(2-(2-(2-((2S,4R)-1-((S)-2-(1-fluorocyclorpropane-1-carboxamido)-3,3-dimethylbutanoyl)-4-hydroxypyrrolidine-2-carboxamido) |
| 101 | methyl)-5-(4-methylthiazol-5-yl)phenoxy)acetamido)acetamido)-4-((2-methoxy-3-(1-methyl-1H-1,2,4-triazol-3-yl)phenyl)amino)- |
| | N-methylpyridazine-3-carboxamide |
| CPD- | 6-(2-(3-(2-(((2S,4R)-1-((S)-2-(1-fluorocyclopropane-1-carboxamido)-3,3-dimethylbutanoyl)-4-hydroxypyrrolidine-2-carboxamido) |
| 102 | methyl)-5-(4-methylthiazol-5-yl)phenoxy)propanamido)acetamido)-4-((2-methoxy-3-(1-methyl-1H-1,2,4-triazol-3-yl)phenyl)amino)- |
| | N-methylpyridazine-3-carboxamide |
| CPD- | 6-(2-(4-(2-(((2S,4R)-1-((S)-2-(1-fluorocyclopropane-1-carboxamido)-3,3-dimethylbutanoyl)-4-hydroxypyrrolidine-2-carboxamido) |
| 103 | methyl)-5-(4-methylthiazol-5-yl)phenoxy)butanamido)acetamido)-4-((2-methoxy-3-(1-methyl-1H-1,2,4-triazol-3-yl)phenyl)amino)- |
| | N-methylpyridazine-3-carboxamide |
| CPD- | 6-(2-(5-(2-(((2S,4R)-1-((S)-2-(1-fluorocyclopropane-1-carboxamido)-3,3-dimethylbutanoyl)-4-hydroxypyrrolidine-2-carboxamido) |
| 104 | methyl)-5-(4-methylthiazol-5-yl)phenoxy)pentanamido)acetamido)-4-((2-methoxy-3-(1-methyl-1H-1,2,4-triazol-3-yl)phenyl)amino)- |
| | N-methylpyridazine-3-carboxamide |
| CPD- | 6-(2-(6-(2-(((2S,4R)-1-((S)-2-(1-fluorocyclopropane-1-carboxamido)-3,3-dimethylbutanoyl)-4-hydroxypyrrolidine-2-carboxamido) |
| 105 | methyl)-5-(4-methylthiazol-5-yl)phenoxy)hexanamido)acetamido)-4-((2-methoxy-3-(1-methyl-1H-1,2,4-triazol-3-yl)phenyl)amino)- |
| | N-methylpyridazine-3-carboxamide |
| CPD- | 6-(2-(7-(2-(((2S,4R)-1-((S)-2-(1-fluorocyclopropane-1-carboxamido)-3,3-dimethylbutanoyl)-4-hydroxypyrrolidine-2-carboxamido) |
| 106 | methyl)-5-(4-methylthiazol-5-yl)phenoxy)heptanamido)acetamido)-4-((2-methoxy-3-(1-methyl-1H-1,2,4-triazol-3-yl)phenyl)amino)- |
| | N-methylpyridazine-3-carboxamide |
| CPD- | 6-(2-(8-(2-(((2S,4R)-1-((S)-2-(1-fluorocyclopropane-1-carboxamido)-3,3-dimethylbutanoyl)-4-hydroxypyrrolidine-2-carboxamido) |
| 107 | methyl)-5-(4-methylthiazol-5-yl)phenoxy)octanamido)acetamido)-4-((2-methoxy-3-(1-methyl-1H-1,2,4-triazol-3-yl)phenyl)amino)- |
| | N-methylpyridazine-3-carboxamide |
| CPD- | 6-(2-(9-(2-(((2S,4R)-1-((S)-2-(1-fluorocyclopropane-1-carboxamido)-3,3-dimethylbutanoyl)-4-hydroxypyrrolidine-2-carboxamido) |
| 108 | methyl)-5-(4-methylthiazol-5-yl)phenoxy)nonanamido)acetamido)-4-((2-methoxy-3-(1-methyl-1H-1,2,4-triazol-3-yl)phenyl)amino)- |
| | N-methylpyridazine-3-carboxamide |
| CPD- | 6-(2-(10-(2-(((2S,4R)-1-((S)-2-(1-fluorocyclopropane-1-carboxamido)-3,3-dimethylbutanoyl)-4-hydroxypyrrolidine-2-carboxamido) |
| 109 | methyl)-5-(4-yl)phenoxy)decanamido)acetamido)-4-((2-methoxy-3-(1-methyl-1H-1,2,4-triazol-3-yl)phenyl)amino)-N- |
| | methylpyridazine-3-carboxamide |
| CPD- | 6-(2-((11-(2-(((2R,4R)-1-((S)-2-(1-fluorocyclopropane-1-carboxamido)-3,3-dimethylbutanoyl)-4-hydroxypyrrolidine-2-carboxamido) |
| 110 | methyl)-5-(4-methylthiazol-5-yl)phenoxy)undecanamido)acetamido)-4-((2-methoxy-3-(1-methyl-1H-1,2,4-triazol-3-yl) |
| | phenyl)amino)-N-methylpyridazine-3-carboxamide |
| CPD- | 6-((5-(4-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)- |
| 111 | 3-oxopropanoyl)piperazin-1-yl)pyridin-2-yl)amino)-4-((2-methoxy-3-(1-methyl-1H-1,2,4-triazol-3-yl)phenyl)amino)-N-methylpyridazine-3- |
| | carboxamide |
| CPD- | 6-((5-(4-(8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan- |
| 112 | 2-yl)amino)-8-oxooctanoyl)piperazin-1-yl)pyridin-2-yl)amino)-4-((2-methoxy-3-(1-methyl-1H-1,2,4-triazol-3-yl)phenyl)amino)-N- |
| | methylpyridazine-3-carboxamide |
| CPD- | 6-((5-(4-(8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan- |
| 113 | 2-yl)amino)-8-oxooctyl)piperazin-1-yl)pyridin-2-yl)amino)-4-((2-methoxy-3-(1-methyl-1H-1,2,4-triazol-3-yl)phenyl)amino)- |
| | N-methylpyridazine-3-carboxamide |
| CPD- | 6-((5-(4-(10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl- |
| 114 | 1-oxobutan-2-yl)amino)-10-oxodecanoyl)piperazin-1-yl)pyridin-2-yl)amino)-4-((2-methoxy-3-(1-methyl-1H-1,2,4-triazol-3-yl) |
| | phenyl)amino)-N-methylpyridazine-3-carboxamide |
| CPD- | 6-((5-(4-(9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 115 | oxobutan-2-yl)amino)-9-oxononanoyl)piperazin-1-yl)pyridin-2-yl)amino)-4-((2-methoxy-3-(1-methyl-1H-1,2,4-triazol-3-yl) |
| | phenyl)amino)-N-methylpyridazine-3-carboxamide |
| CPD- | 6-((5-((1-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 116 | oxobutan-2-yl)amino)-4-oxobutanoyl)piperidin-4-yl)ethynyl)pyridin-2-yl)amino)-4-((2-methoxy-3-(1-methyl-1H-1,2,4-triazol-3-yl) |
| | phenyl)amino)-N-methylpyridazine-3-carboxamide |
| CPD- | 6-((5-((1-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 117 | oxobutan-2-yl)amino)-6-oxohexanoyl)piperidin-4-yl)ethynyl)pyridin-2-yl)amino)-4-((2-methoxy-3-(1-methyl-1H-1,2,4-triazol-3-yl) |
| | phenyl)amino)-N-methylpyridazine-3-carboxamide |
| CPD- | 6-((5-((1-(7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 118 | oxobutan-2-yl)amino)-7-oxoheptanoyl)piperidin-4-yl)ethynyl)pyridin-2-yl)amino)-4-((2-methoxy-3-(1-methyl-1H-1,2,4-triazol-3-yl) |
| | phenyl)amino)-N-methylpyridazine-3-carboxamide |
| CPD- | 6-((5-((1-(8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 119 | oxobutan-2-yl)amino)-8-oxooctanoyl)piperidin-4-yl)ethynyl)pyridin-2-yl)amino)-4-((2-methoxy-3-(1-methyl-1H-1,2,4-triazol-3-yl) |
| | phenyl)amino)-N-methylpyridazine-3-carboxamide |
| CPD- | 6-((5-((1-(9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 120 | oxobutan-2-yl)amino)-9-oxononanoyl)piperidin-4-yl)ethynyl)pyridin-2-yl)amino)-4-((2-methoxy-3-(1-methyl-1H-1,2,4-triazol-3- |
| | yl)phenyl)amino)-N-methylpyridazine-3-carboxamide |
| CPD- | 6-((5-((1-(10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 121 | oxobutan-2-yl)amino)-10-oxodecanoyl)piperidin-4-yl)ethynyl)pyridin-2-yl)amino)-4-((2-methoxy-3-(1-methyl-1H-1,2,4-triazol-3-yl) |
| | phenyl)amino)-N-methylpyridazine-3-carboxamide |
| CPD- | 6-((5-((1-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 122 | oxobutan-2-yl)amino)-4-oxobutanoyl)azetidin-3-yl)ethynyl)pyridin-2-yl)amino)-4-((2-methoxy-3-(1-methyl-1H-1,2,4-triazol-3-yl) |
| | phenyl)amino)-N-methylpyridine-3-carboxamide |
| CPD- | 6-((5-((1-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 123 | oxobutan-2-yl)amino)-6-oxohexanoyl)azetidin-3-yl)ethynyl)pyridin-2-yl)amino)-4-((2-methoxy-3-(1-methyl-1H-1,2,4-triazol-3-yl) |
| | phenyl)amino)-N-methylpyridazine-3-carboxamide |
| CPD- | 6-((5-((1-(7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 124 | oxobutan-2-yl)amino)-7-oxoheptanoyl)azetidin-3-yl)ethynyl)pyridin-2-yl)amino)-4-((2-methoxy-3-(1-methyl-1H-1,2,4-triazol-3-yl) |
| | phenyl)amino)-N-methylpyridazine-3-carboxamide |
| CPD- | 6-((5-((1-(8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 125 | oxobutan-2-yl)amino)-8-oxooctanoyl)azetidin-3-yl)ethynyl)pyridin-2-yl)amino)-4-((2-methoxy-3-(1-methyl-1H-1,2,4-triazol-3-yl) |
| | phenyl)amino)-N-methylpyridazine-3-carboxamide |
| CPD- | 6-((5-((1-(9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 126 | oxobutan-2-yl)amino)-9-oxononanoyl)azetidin-3-yl)ethynyl)pyridin-2-yl)amino)-4-((2-methoxy-3-(1-methyl-1H-1,2,4-triazol-3-yl) |
| | phenyl)amino)-N-methylpyridazine-3-carboxamide |
| CPD- | 6-((5-((1-(10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 127 | oxobutan-2-yl)amino)-10-oxodecanoyl)azetidin-3-yl)ethynyl)pyridin-2-yl)amino)-4-((2-methoxy-3-(1-methyl-1H-1,2,4-triazol-3-yl) |
| | phenyl)amino)-N-methylpyridazine-3-carboxamide |
| CPD- | N1-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2- |
| 128 | yl)-N4-((6-((5-((2-methoxy-3-(1-methyl-1H-1,2,4-triazol-3-yl)phenyl)amino)-6-(methylcarbamoyl)pyridazin-3-yl)amino)pyridin- |
| | 3-yl)methyl)succinamide |
| CPD- | N1-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan- |
| 129 | 2-yl)-N6-((6-((5-((2-methoxy-3-(1-methyl-1H-1,2,4-triazol-3-yl)phenyl)amino)-6-(methylcarbamoyl)pyridazin-3-yl)amino)pyridin- |
| | 3-yl)methyl)adipamide |
| CPD- | N1-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan- |
| 130 | 2-yl)-N7-((6-((5-((2-methoxy-3-(1-methyl-1H-1,2,4-triazol-3-yl)phenyl)amino)-6-(methylcarbamoyl)pyridazin-3-yl)amino)pyridin- |
| | 3-yl)methyl)heptanediamide |
| CPD- | N1-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)- |
| 131 | N8-((6-((5-((2-methoxy-3-(1-methyl-1H-1,2,4-triazol-3-yl)phenyl)amino)-6-(methylcarbamoyl)pyridazin-3-yl)amino)pyridin-3-yl)methyl) |
| | octanediamide |
| CPD- | N1-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-N9- |
| 132 | ((6-((5-((2-methoxy-3-(1-methyl-1H-1,2,4-triazol-3-yl)phenyl)amino)-6-(methylcarbamoyl)pyridazin-3-yl)amino)pyridin-3-yl)methyl) |
| | nonanediamide |
| CPD- | N1-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)- |
| 133 | N10-((6-((5-((2-methoxy-3-(1-methyl-1H-1,2,4-triazol-3-yl)phenyl)amino)-6-(methylcarbamoyl)pyridazin-3-yl)amino)pyridin-3-yl)methyl) |
| | decanediamide |
| CPD- | 6-((5-((1-(5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 134 | oxobutan-2-yl)amino)-5-oxopentanoyl)piperidin-4-yl)ethynyl)pyridin-2-yl)amino)-4-((2-methoxy-3-(1-methyl-1H-1,2,4-triazol-3-yl) |
| | phenyl)amino)-N-methylpyridazine-3-carboxamide |
| CPD- | 6-((5-((1-(5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 135 | oxobutan-2-yl)amino)-5-oxopentanoyl)azetidin-3-yl)ethynyl)pyridin-2-yl)amino)-4-((2-methoxy-3-(1-methyl-1H-1,2,4-triazol-3-yl) |
| | phenyl)amino)-N-methylpyridazine-3-carboxamide |
| CPD- | N1-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)- |
| 136 | N5-((6-((5-((2-methoxy-3-(1-methyl-1H-1,2,4-triazol-3-yl)phenyl)amino)-6-(methylcarbamoyl)pyridazin-3-yl)amino)pyridin-3-yl)methyl) |
| | glutaramide |
| CPD- | 6-((5-((2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2- |
| 137 | yl)amino)-2-oxoethyl)carbamoyl)pyridin-2-yl)amino)-4-((2-methoxy-3-(1-methyl-1H-1,2,4-triazol-3-yl)phenyl)amino)-N-methylpyridazine-3- |
| | carboxamide |
| CPD- | 6-((5-((3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2- |
| 138 | yl)amino)-3-oxopropyl)carbamoyl)pyridin-2-yl)amino)-4-((2-methoxy-3-(1-methyl-1H-1,2,4-triazol-3-yl)phenyl)amino)-N-methylpyridazine- |
| | 3-carboxamide |
| CPD- | 6-((5-((4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2- |
| 139 | yl)amino)-4-oxobutyl)carbamoyl)pyridin-2-yl)amino)-4-((2-methoxy-3-(1-methyl-1H-1,2,4-triazol-3-yl)phenyl)amino)-N-methylpyridazine |
| | 3-carboxamide |
| CPD- | 6-((5-((5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2- |
| 140 | yl)amino)-5-oxopentyl)carbamoyl)pyridin-2-yl)amino)-4-((2-methoxy-3-(1-methyl-1H-1,2,4-triazol-3-yl)phenyl)amino)-N-methylpyridazine |
| | 3-carboxamide |
| CPD- | 6-((5-((6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2- |
| 141 | yl)amino)-6-oxohexyl)carbamoyl)pyridin-2-yl)amino)-4-((2-methoxy-3-(1-methyl-1H-1,2,4-triazol-3-yl)phenyl)amino)-N-methylpyridazine-3- |
| | carboxamide |
| CPD- | 6-((5-((7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2- |
| 142 | yl)amino)-7-oxoheptyl)carbamoyl)pyridin-2-yl)amino)-4-((2-methoxy-3-(1-methyl-1H-1,2,4-triazol-3-yl)phenyl)amino)-N-methylpyridazine- |
| | 3-carboxamide |
| CPD- | 6-((5-((8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 143 | oxobutan-2-yl)amino)-8-oxooctyl)carbamoyl)pyridin-2-yl)amino)-4-((2-methoxy-3-(1-methyl-1H-1,2,4-triazol-3-yl)phenyl)amino)- |
| | N-methylpyridazine-3-carboxamide |
| CPD- | 6-((5-((9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 144 | oxobutan-2-yl)amino)-9-oxononyl)carbamoyl)pyridin-2-yl)amino)-4-((2-methoxy-3-(1-methyl-1H-1,2,4-triazol-3-yl)phenyl)amino)- |
| | N-methylpyridazine-3-carboxamide |
| CPD- | 6-((5-(4-(1-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 145 | oxobutan-2-yl)amino)-4-oxobutanoyl)piperidin-4-yl)piperazin-1-yl)pyridin-2-yl)amino)-4-((2-methoxy-3-(1-methyl-1H-1,2,4- |
| | triazol-3-yl)phenyl)-amino)-N-methylpyridazine-3-carboxamide |
| CPD- | 6-((5-(4-(1-(5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 146 | oxobutan-2-yl)amino)-5-oxopentanoyl)piperidin-4-yl)piperazin-1-yl)pyridin-2-yl)amino)-4-((2-methoxy-3-(1-methyl-1H-1,2,4-triazol- |
| | 3-yl)phenyl)amino)-N-methylpyridazine-3-carboxamide |
| CPD- | 6-((5-(4-(1-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 147 | oxobutan-2-yl)amino)-6-oxohexanoyl)piperidin-4-yl)piperazin-1-yl)pyridin-2-yl)amino)-4-((2-methoxy-3-(1-methyl-1H-1,2,4-triazol-3-yl) |
| | phenyl)amino)-N-methylpyridazine-3-carboxamide |
| CPD- | 6-((5-(4-(1-(7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl- |
| 148 | 1-oxobutan-2-yl)amino)-7-oxoheptanoyl)piperidin-4-yl)piperazin-1-yl)pyridin-2-yl)amino)-4-((2-methoxy-3-(1-methyl-1H-1,2,4-triazol-3- |
| | yl)phenyl)amino)-N-methylpyridazine-3-carboxamide |
| CPD- | 6-((5-(4-(1-(8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 149 | oxobutan-2-yl)amino)-8-oxooctanoyl)piperidin-4-yl)piperazin-1-yl)pyridin-2-yl)amino)-4-((2-methoxy-3-(1-methyl-1H-1,2,4-triazol-3-yl) |
| | phenyl)amino)-N-methylpyridazine-3-carboxamide |
| CPD- | 6-((5-(4-(1-(9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 150 | oxobutan-2-yl)amino)-9-oxononanoyl)piperidin-4-yl)piperazin-1-yl)pyridin-2-yl)amino)-4-((2-methoxy-3-(1-methyl-1H-1,2,4-triazol-3- |
| | yl)phenyl)amino)-N-methylpyridazine-3-carboxamide |
| CPD- | 6-((5-(4-(1-(10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 151 | oxobutan-2-yl)amino)-10-oxodecanoyl)piperidin-4-yl)piperazin-1-yl)pyridin-2-yl)amino)-4-((2-methoxy-3-(1-methyl-1H-1,2,4-triazol- |
| | 3-yl)phenyl)amino)-N-methylpyridazine-3-carboxamide |
| CPD- | 6-((5-((2-((2-(2,6-dioxopiperidin-3-yl)-1,3-dioxoisoindolin-4-yl)amino)ethyl)carbamoyl)pyridin-2-yl)amino)-4-((2-methoxy-3-(1-methyl-1H- |
| 152 | 1,2,4-triazol-3-yl)phenyl)amino)-N-methylpyridazine-3-carboxamide |
| CPD- | 6-((5-((3-((2-(2,6-dioxopiperidin-3-yl)-1,3-dioxoisindolin-4-yl)amino)propyl)carbamoyl)pyridin-2-yl)amino)-4-((2-methoxy-3-(1-methyl-1H- |
| 153 | 1,2,4-triazol-3-yl)phenyl)amino)-N-methylpyridazine-3-carboxamide |
| CPD- | 6-((5-((4-((2-(2,6-dioxopiperidin-3-yl)-1,3-dioxoisoindolin-4-yl)amino)butyl)carbamoyl)pyridin-2-yl)amino)-4-((2-methoxy-3-(1-methyl-1H- |
| 154 | 1,2,4-triazol-3-yl)phenyl)amino)-N-methylpyridazine-3-carboxamide |
| CPD- | 6-((5-((5-((2-(2,6-dioxopiperidin-3-yl)-1,3-dioxoisoindolin-4-yl)amino)pentyl)carbamoyl)pyridin-2-yl)amino)-4-((2-methoxy-3-(1-methyl-1H- |
| 155 | 1,2,4-triazol-3-yl)phenyl)amino)-N-methylpyridazine-3-carboxamide |
| CPD- | 6-((5-((6-((2-(2,6-dioxopiperidin-3-yl)-1,3-dioxoisoindolin-4-yl)amino)hexyl)carbamoyl)pyridin-2-yl)amino)-4-((2-methoxy-3-(1-methyl-1H- |
| 156 | 1,2,4-triazol-3-yl)phenyl)amino)-N-methylpyridazine-3-carboxamide |
| CPD- | 6-((5-((7-((2-(2,6-dioxopiperidin-3-yl)-1,3-dioxoisoindolin-4-yl)amino)heptyl)carbamoyl)pyridin-2-yl)amino)-4-((2-methoxy-3-(1-methyl-1H- |
| 157 | 1,2,4-triazol-3-yl)phenyl)amino)-N-methylpyridazine-3-carboxamide |
| CPD- | 6-((5-((8-((2-(2,6-dioxopiperidin-3-yl)-1,3-dioxoisoindolin-4-yl)amino)octyl)carbamoyl)pyridin-2-yl)amino)-4-((2-methoxy-3-(1-methyl-1H- |
| 158 | 1,2,4-triazol-3-yl)phenyl)amino)-N-methylpyridazine-3-carboxamide |
| CPD- | 6-((5-((2-(2-((2-(2,6-dioxopiperidin-3-yl)-1,3-dioxoisoindolin-4-yl)amino)ethoxy)ethyl)carbamoyl)pyridin-2-yl)amino)-4-((2-methoxy-3-(1- |
| 159 | methyl-1H-1,2,4-triazol-3-yl)phenyl)amino)-N-methylpyridazine-3-carboxamide |
| CPD- | 6-((5-((1-(5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 160 | oxobutan-2-yl)amino)-5-oxopentyl)piperidin-4-yl)ethynyl)pyridin-2-yl)amino)-4-((2-methoxy-3-(1-methyl-1H-1,2,4-triazol-3-yl) |
| | phenyl)amino)-N-methylpyridazine-3-carboxamide |
| CPD- | 6-((5-((3-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 161 | oxobutan-2-yl)amino)-2-oxoethyl)cyclobutyl)carbamoyl)pyridin-2-yl)amino)-4-((2-methoxy-3-(1-methyl-1H-1,2,4-triazol-3-yl) |
| | phenyl)amino)-N-methylpyridazine-3-carboxamide |
| CPD- | 6-((5-(((8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 162 | oxobutan-2-yl)amino)-8-oxooctyl)amino)methyl)pyridin-2-yl)amino)-4-((2-methoxy-3-(1-methyl-1H-1,2,4-triazol-3-yl)phenyl) |
| | amino)-N-methylpyridazine-3-carboxamide |
| CPD- | 6-((5-((4-((2-(2,6-dioxopiperidin-3-yl)-1,3-dioxoisoindolin-5-yl)amino)butyl)carbamoyl)pyridin-2-yl)amino)-4-((2-methoxy-3-(1-methyl-1H- |
| 163 | 1,2,4-triazol-3-yl)phenyl)amino)-N-methylpyridazine-3-carboxamide |
| CPD- | 6-((5-((5-((2-(2,6-dioxopiperidin-3-yl)-1,3-dioxoisoindolin-5-yl)amino)pentyl)carbamoyl)pyridin-2-yl)amino)-4-((2-methoxy-3-(1-methyl-1H- |
| 164 | 1,2,4-triazol-3-yl)phenyl)amino)-N-methylpyridazine-3-carboxamide |
| CPD- | 6-((5-((6-((2-(2,6-dioxopiperidin-3-yl)-1,3-dioxoisoindolin-5-yl)amino)hexyl)carbamoyl)pyridin-2-yl)amino)-4-((2-methoxy-3-(1-methyl-1H- |
| 165 | 1,2,4-triazol-3-yl)phenyl)amino)-N-methylpyridazine-3-carboxamide |
| CPD- | 6-((5-((7-((2-(2,6-dioxopiperidin-3-yl)-1,3-dioxoisoindolin-5-yl)amino)heptyl)carbamoyl)pyridin-2-yl)amino)-4-((2-methoxy-3-(1-methyl-1H- |
| 166 | 1,2,4-triazol-3-yl)phenyl)amino)-N-methylpyridazine-3-carboxamide |
| CPD- | 6-((5-((8-((2-(2,6-dioxopiperidin-3-yl)-1,3-dioxoisoindolin-5-yl)amino)octyl)carbamoyl)pyridin-2-yl)amino)-4-((2-methoxy-3-(1-methyl-1H- |
| 167 | 1,2,4-triazol-3-yl)phenyl)amino)-N-methylpyridazine-3-carboxamide |
| CPD- | 6-((5-((2-(2-((2-(2,6-dioxopiperidin-3-yl)-1,3-dioxoisoindolin-5-yl)amino)ethoxy)ethyl)carbamoyl)pyridin-2-yl)amino)-4-((2-methoxy-3-(1- |
| 168 | methyl-1H-1,2,4-triazol-3-yl)phenyl)amino)-N-methylpyridazine-3-carboxamide |
| CPD- | 6-((5-(4-(8-(((S)-1-((2S,4R)-4-hydroxy-2-(((S)-1-phenylethyl)carbamoyl)pyrrolidin-1-yl)-3,3-dimethyl-1-oxobutan-2-yl)amino)-8- |
| 169 | oxooctanoyl)piperazin-1-yl)pyridin-2-yl)amino)-4-((2-methoxy-3-(1-methyl-1H-1,2,4-triazol-3-yl)phenyl)amino)-N-methylpyridazine-3- |
| | carboxamide |
| CPD- | 6-((5-(4-(8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan- |
| 170 | 2-yl)amino)-8-oxooctanoyl)piperazin-1-yl)pyridin-2-yl)amino)-4-((2-methoxyphenyl)amino)-N-methylnicotinamide |
| CPD- | 6-((5-(1-(8-(((S)-1-((2S,4R)-2-((4-cyanobenzyl)carbamoyl)-4-hydroxypyrrolidin-1-yl)-3,3-dimethyl-1-oxobutan-2-yl)amino)-8-oxooctanoyl) |
| 171 | piperidin-4-yl)pyridin-2-yl)amino)-4-((2-methoxy-3-(1-methyl-1H-1,2,4-triazol-3-yl)phenyl)amino)-N-methylpyridazine-3-carboxamide |
| CPD- | 6-((5-(4-(8-(((S)-1-((2S,4R)-2-(((5-cyanopyridin-2-yl)methyl)carbamoyl)-4-hydroxypyrrolidin-1-yl)-3,3-dimethyl-1-oxobutan-2-yl)amino)-8- |
| 172 | oxooctanoyl)piperazin-1-yl)pyridin-2-yl)amino)-4-((2-methoxy-3-(1-methyl-1H-1,2,4-triazol-3-yl)phenyl)amino)-N-methylpyridazine-3- |
| | carboxamide |
| CPD- | 6-((5-(4-(8-(((S)-1-((2S,4R)-2-(((S)-2-cyano-1-methyl-4,5,6,7-tetrahydro-1H-indol-4-yl)carbamoyl)-4-hydroxypyrrolidin-1-yl)-3,3-dimethyl- |
| 173 | 1-oxobutan-2-yl)amino)-8-oxooctanoyl)piperazin-1-yl)pyridin-2-yl)amino)-4-((2-methoxy-3-(1-methyl-1H-1,2,4-triazol-3-yl)phenyl)amino)- |
| | N-methylpyridazine-3-carboxamide |
| CPD- | 6-((5-(4-(8-(((S)-1-((2S,4R)-4-hydroxy-2-(((S)-3-methyl-2-oxo-1,2,5,6,7,8-hexahydroquinolin-5-yl)carbamoyl)pyrrolidin-1-yl)-3,3-dimethyl- |
| 174 | 1-oxobutan-2-yl)amino)-8-oxooctanoyl)piperazin-1-yl)pyridin-2-yl)amino)-4-((2-methoxy-3-(1-methyl-1H-1,2,4-triazol-3-yl) |
| | phenyl)amino)-N-methylpyridazine-3-carboxamide |
| CPD- | 6-((5-((3-((2-(2,6-dioxopiperidin-3-yl)-1,3-dioxoisoindolin-5-yl)amino)propyl)carbamoyl)pyridin-2-yl)amino)-4-((2-methoxy-3-(1-methyl-1H- |
| 175 | 1,2,4-triazol-3-yl)phenyl)amino)-N-methylpyridazine-3-carboxamide |
| CPD- | 6-((5-((2-((2-(2,6-dioxopiperidin-3-yl)-1,3-dioxoisoindolin-5-yl)amino)ethyl)carbamoyl)pyridin-2-yl)amino)-4-((2-methoxy-3-(1-methyl-1H- |
| 176 | 1,2,4-triazol-3-yl)phenyl)amino)-N-methylpyridazine-3-carboxamide |
| CPD- | 6-((5-(4-(8-(((S)-1-((2S,4R)-2-(((S)-1-(4-cyanophenyl)ethyl)carbamoyl)-4-hydroxypyrrolidin-1-yl)-3,3-dimethyl-1-oxobutan-2-yl)amino)-8- |
| 177 | oxooctanoyl)piperazin-1-yl)pyridin-2-yl)amino)-4-((2-methoxy-3-(1-methyl-1H-1,2,4-triazol-3-yl)phenyl)amino)-N-methylpyridazine-3- |
| | carboxamide |
| CPD- | 6-((5-(4-(8-(((S)-1-((2S,4R)-2-((4-cyanobenzyl)carbamoyl)-4-hydroxypyrrolidin-1-yl)-3,3-dimethyl-1-oxobutan-2-yl)amino)-8-oxooctanoyl) |
| 178 | piperazin-1-yl)pyridin-2-yl)amino)-4-((2-methoxy-3-(1-methyl-1H-1,2,4-triazol-3-yl)phenyl)amino)pyridazine-3-carboxamide |
| CPD- | 6-((5-(4-(8-(((S)-1-((2S,4R)-2-((4-cyanobenzyl)carbamoyl)-4-hydroxypyrrolidin-1-yl)-3,3-dimethyl-1-oxobutan-2-yl)amino)-8- |
| 179 | oxooctanoyl)piperazin-1-yl)pyridin-2-yl)amino)-4-((2-methoxy-3-(1-methyl-1H-1,2,4-triazol-3-yl)phenyl)amino)-N-(methyl-d3)pyridazine-3- |
| | carboxamide |
| CPD- | 6-((5-(4-(8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 180 | oxobutan-2-yl)amino)-8-oxooctanoyl)piperazin-1-yl)pyridin-2-yl)amino)-4-((2-methoxy-3-(1-methyl-1H-1,2,4-triazol-3-yl)phenyl)amino) |
| | pyridazine-3-carboxamide |
| CPD- | 6-((5-(4-(8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 181 | oxobutan-2-yl)amino)-8-oxooctanoyl)piperazin-1-yl)pyridin-2-yl)amino)-4-((2-methoxy-3-(1-methyl-1H-1,2,4-triazol-3-yl)phenyl) |
| | amino)-N-(methyl-d3)pyridazine-3-carboxamide |
| CPD- | 6-((5-((1-(5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 182 | oxobutan-2-yl)amino)-5-oxopentanoyl)piperidin-4-yl)ethynyl)pyridin-2-yl)amino)-4-((2-methoxy-3-(1-methyl-1H-1,2,4-triazol-3-yl) |
| | phenyl)amino)pyridazine-3-carboxamide |
| CPD- | 6-((5-((1-(5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 183 | oxobutan-2-yl)amino)-5-oxopentanoyl)piperidin-4-yl)ethynyl)pyridin-2-yl)amino)-4-((2-methoxy-3-(1-methyl-1H-1,2,4-triazol-3-yl) |
| | phenyl)amino)-N-(methyl-d3)pyridazine-3-carboxamide |
| CPD- | 6-((5-((1-(10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 184 | oxobutan-2-yl)amino)-10-oxodecanoyl)piperidin-4-yl)ethynyl)pyridin-2-yl)amino)-4-((2-methoxy-3-(1-methyl-1H-1,2,4-triazol-3-yl) |
| | phenyl)amino)pyridazine-3-carboxamide |
| CPD- | 6-((5-((1-(10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 185 | oxobutan-2-yl)amino)-10-oxodecanoyl)piperidin-4-yl)ethynyl)pyridin-2-yl)amino)-4-((2-methoxy-3-(1-methyl-1H-1,2,4-triazol-3-yl) |
| | phenyl)amino)-N-(methyl-d3)pyridazine-3-carboxamide |
| CPD- | 6-((5-((1-(7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl- |
| 186 | 1-oxobutan-2-yl)amino)-7-oxoheptanoyl)azetidin-3-yl)ethynyl)pyridin-2-yl)amino)-4-((2-methoxy-3-(1-methyl-1H-1,2,4-triazol-3-yl) |
| | phenyl)amino)pyridazine-3-carboxamide |
| CPD- | 6-((5-((1-(7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 187 | oxobutan-2-yl)amino)-7-oxoheptanoyl)azetidin-3-yl)ethynyl)pyridin-2-yl)amino)-4-((2-methoxy-3-(1-methyl-1H-1,2,4-triazol-3-yl) |
| | phenyl)amino)-N-(methyl-d3)pyridazine-3-carboxamide |
| CPD- | 6-((5-(4-(1-(10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl- |
| 188 | 1-oxobutan-2-yl)amino)-10-oxodecanoyl)piperidin-4-yl)piperazin-1-yl)pyridin-2-yl)amino)-4-((2-methoxy-3-(1-methyl-1H-1,2,4- |
| | triazol-3-yl)phenyl)amino)pyridazine-3-carboxamide |
| CPD- | 6-((5-(4-(1-(10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl- |
| 189 | 1-oxobutan-2-yl)amino)-10-oxodecanoyl)piperidin-4-yl)piperazin-1-yl)pyridin-2-yl)amino)-4-((2-methoxy-3-(1-methyl-1H-1,2,4-triazol- |
| | 3-yl)phenyl)amino)-N-(methyl-d₃)pyridazine-3-carboxamide |
| CPD- | N¹-((6-((6-carbamoyl-5-((2-methoxy-3-(1-methyl-1H-1,2,4-triazol-3-yl)phenyl)amino)pyridazin-3-yl)amino)pyridin-3-yl)methyl-N⁸-((S)-1- |
| 190 | ((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)octanediamide |
| CPD- | N¹-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-N⁸- |
| 191 | ((6-((5-((2-methoxy-3-(1-methyl-1H-1,2,4-triazol-3-yl)phenyl)amino)-6-((methyl-d₃)carbamoyl)pyridazin-3-yl)amino)pyridin-3-yl)methyl) |
| | octanediamide |
| CPD- | N¹-((6-((6-carbamoyl-5-((2-methoxy-3-(1-methyl-1H-1,2,4-triazol-3-yl)phenyl)amino)pyridazin-3-yl)amino)pyridin-3-yl)methyl)-N¹⁰-((S)-1- |
| 192 | ((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)decanediamide |
| CPD- | N¹-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-N¹⁰- |
| 193 | ((6-((5-((2-methoxy-3-(1-methyl-1H-1,2,4-triazol-3-yl)phenyl)amino)-6-((methyl-d₃)carbamoyl)pyridazin-3-yl)amino)pyridin-3-yl)methyl) |
| | decanediamide |
| CPD- | 6-((5-((9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1- |
| 194 | oxobutan-2-yl)amino)-9-oxononyl)carbamoyl)pyridin-2-yl)amino)-4-((2-methoxy-3-(1-methyl-1H-1,2,4-triazol-3-yl)phenyl)amino) |
| | pyridazine-3-carboxamide |
| CPD- | 6-((5-((9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2- |
| 195 | yl)amino)-9-oxononyl)carbamoyl)pyridin-2-yl)amino)-4-((2-methoxy-3-(1-methyl-1H-1,2,4-triazol-3-yl)phenyl)amino)-N-(methyl-d3) |
| | pyridazine-3-carboxamide |
| CPD- | 6-((5-((5-((2-(2,6-dioxopiperidin-3-yl)-1,3-dioxoisoindolin-5-yl)amino)pentyl)carbamoyl)pyridin-2-yl)amino)-4-((2-methoxy-3-(1-methyl- |
| 196 | 1H-1,2,4-triazol-3-yl)phenyl)amino)pyridazine-3-carboxamide |
| CPD- | 6-((5-((5-((2-(2,6-dioxopiperidin-3-yl)-1,3-dioxoisoindolin-4-yl)amino)pentyl)carbamoyl)pyridin-2-yl)amino)-4-((2-methoxy-3-(1-methyl- |
| 197 | 1H-1,2,4-triazol-3-yl)phenyl)amino)-N-(methyl-d3)pyridazine-3-carboxamide |
| CPD- | 6-((5-((5-((2-(2,6-dioxopiperidin-3-yl)-1,3-dioxoisoindolin-5-yl)amino)pentyl)carbamoyl)pyridin-2-yl)amino)-4-((2-methoxy-3-(1-methyl- |
| 198 | 1H-1,2,4-triazol-3-yl)phenyl)amino)pyridazine-3-carboxamide |
| CPD- | 6-((5-((5-((2-(2,6-dioxopiperidin-3-yl)-1,3-dioxoisoindolin-5-yl)amino)pentyl)carbamoyl)pyridin-2-yl)amino)-4-((2-methoxy-3-(1-methyl- |
| 199 | 1H-1,2,4-triazol-3-yl)phenyl)amino)pyridazine-3-carboxamide |

As used herein, in case of discrepancy between the structure and chemical name provided for a particular compound, the structure shall control.

### Example 239. Selected TYK2 Degraders Reduced TYK2 Protein Levels in MOLT-4 Cells (Tables. 2 and 3)

MOLT-4 cells were treated with compounds at 0.5 and 5 μM concentrations for 24 hours. Data showed that selected compounds reduced TYK2 proteins levels.

### Example 240. Selected TYK2 Degraders Selectively Reduced TYK2 Protein Levels in MOLT-4 Cells (FIG. 1)

MOLT-4 cells were treated with CPD-038, CPD-039, and CPD-040 at 0.04, 0.2, 1, and 5 μM concentrations for 24 hours. CPD-038, CPD-039, and CPD-040 concentration-dependently reduced the TYK2 proteins levels. Data shown that CPD-038, CPD-039, and CPD-040 are very selective at the reduction protein levels of TYK2 over JAK1, JAK2, and JAK3.

### Example 241. Selected TYK2 Degraders Potently Reduced TYK2 Protein Levels in NOMO-1 Cells (FIG. 2 and Table 3)

NOMO-1 cells were treated with CPD-155, CPD-157, and CPD-158 at 0.1, 1, 10, 100 and 1000 nM concentrations for 16 hours. CPD-155, CPD-157, and CPD-158 concentration-dependently reduced the TYK2 proteins levels. Data shown that CPD-155, CPD-157, and CPD-158 are very potent at the reduction protein levels of TYK2. Hook effect was observed in all three heterobifunctional compounds.

### Example 242. Selected TYK2 Degraders Effectively Inhibited Phosphorylation of STAT 1/3 in Response to IFNα Treatment, and Reduced TYK2 Protein Levels in Jurkat Cells (FIG. 3)

Jurkat cells were treated with DMSO or a dose range of heterobifunctional compounds CPD-155, CPD-158, and CPD-164 at 3 or 30 nM concentrations for 16 hours. Cells were subsequently treated with IFNα (2000 IU) for 15 min as indicated. CPD-155, CPD-158, and CPD-164 concentration-dependently reduced phosphorylation of STAT1 (Tyr701) and STAT3 (Tyr705) induced by IFNα treatment, which correlated with the reduced levels of TYK2 proteins. Data showed that multiple heterobifunctional compounds effectively suppressed IFNα-induced STAT1/3 phosphorylation along with reduction of TYK2 protein levels.

Materials and Methods:

General Chemistry Methods:

All chemicals and reagents were purchased from commercial suppliers and used without further purification. LCMS spectra for all compounds were acquired using a Waters LC-MS AcQuity H UPLC class system. The Waters LC-MS AcQuity H UPLC class system comprising a pump (Quaternary Solvent Manager) with degasser, an autosampler (FTN), a column oven (40° C., unless otherwise indicated), a photo-diode array PDA detector. Chromatography was performed on an AcQuity UPLC BEH C18 (1.7 m, 2.1×50 mm) with water containing 0.1% formic acid as solvent A and acetonitrile containing 0.1% formic acid as solvent B at a flow rate of 0.6 mL/min. Flow from the column was split to a MS spectrometer. The MS detector was configured with an electrospray ionization source. Nitrogen was used as the nebulizer gas. Data acquisition was performed with a MassLynx data system. Nuclear Magnetic Resonance spectra were recorded on a Bruker Avance III 400 spectrometer. Chemical shifts are expressed in parts per million (ppm) and reported as δ value (chemical shift 6). Coupling constants are reported in units of hertz (J value, Hz; Integration and splitting patterns: where s=singlet, d=double, t=triplet, q=quartet, brs=broad singlet, m=multiple). The purification of intermediates or final products were performed on Agilent Prep 1260 series with UV detector set to 254 nm or 220 nm. Samples were injected onto a Phenomenex Luna C18 column (5 m, 30×75 mm,) at room temperature. The flow rate was 40 mL/min. A linear gradient was used with either 10% or 50% MeOH in H₂O containing 0.1% TFA as solvent A and 100% of MeOH as solvent B. Alternatively, the products were purified on CombiFlash® NextGen 300 system with UV detector set to 254 nm, 220 nm or 280 nm. The flow rate was 40 mL/min. A linear gradient was used with H₂O containing 0.05% TFA as solvent A and 100% of MeOH containing 0.05% TFA as solvent B. All compounds showed >95% purity using the LCMS methods described above.

Cell Culture

MOLT-4 or NOMO-1 cells were cultured at 37° C. with 5% CO₂ in RPMI 1640 Medium supplemented with 10% fetal bovine serum. Cells were authenticated using the short tandem repeat (STR) assays. Mycoplasma test results were negative.

Antibodies and Reagents

Rabbit anti-JAK1antibody (3344S), anti-JAK2 antibody (3230S), anti-JAK3 antibody (8827S), anti-TYK2 antibody (14193S), anti-STAT1 antibody (9167S), anti-STAT3 antibody (9139S), anti-phospho-STAT1 (Tyr701) antibody (9167S), anti-phospho-STAT3 (Tyr705) antibody (9145S), were purchased from Cell Signaling Technology; anti-β-actin antibody (ab8226) was from Abeam; HRP-conjugated anti-α-Tubulin (GNI4310-AT) antibody was purchased from GNI. Media and other cell culture reagents were purchased from Thermo Fisher.

Immunoblotting

Cultured cells were washed with cold PBS once and lysed in cold RIPA buffer supplemented with protease inhibitors and phosphatase inhibitors (Beyotime Biotechnology). The solutions were then incubated at 4° C. for 30 minutes with gentle agitation to fully lyse cells. Cell lysates were centrifuged at 13,000 rpm for 10 minutes at 4° C. and pellets were discarded. Total protein concentrations in the lysates were determined by BCA assays (Beyotime Biotechnology). Cell lysates were mixed with Laemmli loading buffer to 1× and heated at 99° C. for 5 min. Proteins were resolved on SDS-PAGE and visualized by chemiluminescence. Images were taken by a ChemiDoc MP Imaging system (Bio-Rad). Protein bands were quantitated using the accompanied software provided by Bio-Rad.

The TYK2 protein degradation results of selected heterobifunctional compounds are set forth in Tables 2 and 3 below.

TABLE 2

| | TYK2 Degradation | TYK2 Degradation |
|---|---|---|
| Cmpd. | (0.5 μM) | (5 μM) |
| CPD-001 | ≤20% | |
| CPD-002 | ≤20% | |
| CPD-003 | ≤20% | |
| CPD-004 | ≤20% | |
| CPD-005 | ≤20% | |
| CPD-006 | ≤20% | |
| CPD-007 | ≤20% | |
| CPD-008 | ≤20% | |
| CPD-009 | ≤20% | |
| CPD-010 | ≤20% | |
| CPD-011 | ≤20% | |
| CPD-012 | ≤20% | |
| CPD-013 | ≤20% | |
| CPD-014 | ≤20% | |
| CPD-015 | ≤20% | |
| CPD-016 | ≤20% | |
| CPD-017 | ≤20% | |
| CPD-018 | ≤20% | |
| CPD-019 | ≤20% | |
| CPD-020 | ≤20% | |
| CPD-021 | ≤20% | ≤20% |
| CPD-022 | 40% | ≤20% |
| CPD-023 | ≤20% | ≤20% |
| CPD-024 | ≤20% | 30% |
| CPD-025 | ≤20% | ≤20% |
| CPD-026 | ≤20% | ≤20% |
| CPD-027 | ≤20% | ≤20% |
| CPD-028 | ≤20% | ≤20% |
| CPD-029 | ≤20% | ≤20% |
| CPD-030 | ≤20% | ≤20% |
| CPD-031 | ≤20% | ≤20% |
| CPD-032 | ≤20% | ≤20% |
| CPD-033 | ≤20% | ≤20% |
| CPD-034 | ≤20% | ≤20% |
| CPD-035 | ≤20% | ≤20% |
| CPD-036 | ≤20% | 40% |
| CPD-037 | ≤20% | 30% |
| CPD-038 | 50% | 90% |
| CPD-039 | 60% | 90% |
| CPD-040 | 60% | 60% |
| CPD-041 | 50% | 30% |
| CPD-042 | ≤20% | ≤20% |
| CPD-043 | ≤20% | ≤20% |
| CPD-044 | ≤20% | ≤20% |
| CPD-045 | 40% | ≤20% |
| CPD-046 | ≤20% | ≤20% |
| CPD-047 | 60% | ≤20% |
| CPD-048 | 50% | ≤20% |
| CPD-049 | ≤20% | ≤20% |
| CPD-050 | ≤20% | ≤20% |
| CPD-051 | ≤20% | ≤20% |
| CPD-052 | ≤20% | ≤20% |
| CPD-053 | ≤20% | ≤20% |
| CPD-054 | ≤20% | ≤20% |
| CPD-055 | ≤20% | ≤20% |
| CPD-056 | ≤20% | ≤20% |
| CPD-057 | ≤20% | ≤20% |
| CPD-058 | ≤20% | ≤20% |
| CPD-059 | ≤20% | ≤20% |
| CPD-060 | ≤20% | ≤20% |
| CPD-061 | ≤20% | ≤20% |
| CPD-062 | ≤20% | ≤20% |
| CPD-063 | ≤20% | 30% |
| CPD-064 | ≤20% | ≤20% |
| CPD-065 | ≤20% | ≤20% |
| CPD-066 | ≤20% | ≤20% |
| CPD-067 | ≤20% | ≤20% |
| CPD-068 | ≤20% | ≤20% |
| CPD-069 | ≤20% | ≤20% |
| CPD-070 | ≤20% | ≤20% |
| CPD-071 | ≤20% | ≤20% |
| CPD-072 | ≤20% | ≤20% |
| CPD-073 | ≤20% | ≤20% |
| CPD-074 | ≤20% | ≤20% |
| CPD-075 | ≤20% | ≤20% |
| CPD-076 | ≤20% | ≤20% |
| CPD-077 | ≤20% | ≤20% |
| CPD-078 | ≤20% | ≤20% |

The percentage of TYK2 protein degradation of each compound at 0.5 and 5 μM determined in MOLT-4 cells as described in Methods.

TABLE 3

| | TYK2 | TYK2 | TYK2 | TYK2 |
|---|---|---|---|---|
| | Degradation | Degradation | Degradation | Degradation |
| | (MOLT-4, | (MOLT-4, | (NOMO-1, | (NOMO-1, |
| Cmpd. | 0.5 μM) | 5 μM) | 10 nM) | 100 nM) |
| CPD-079 | C | C | | |
| CPD-080 | C | C | | |
| CPD-081 | C | C | | |
| CPD-082 | C | C | | |
| CPD-083 | B | A | | |
| CPD-084 | A | A | | |
| CPD-085 | A | A | | |
| CPD-086 | C | | | |
| CPD-087 | C | | | |
| CPD-088 | C | | | |
| CPD-089 | C | C | | |
| CPD-090 | C | C | | |
| CPD-091 | C | C | | |
| CPD-092 | C | C | | |
| CPD-093 | C | C | | |
| CPD-094 | B | B | | |
| CPD-095 | B | A | | |
| CPD-096 | B | C | | |
| CPD-097 | B | C | | |
| CPD-098 | C | C | | |
| CPD-099 | A | B | | |
| CPD-100 | A | B | | |
| CPD-101 | C | C | | |
| CPD-102 | C | C | | |
| CPD-103 | C | C | | |
| CPD-104 | C | C | | |
| CPD-105 | C | C | | |
| CPD-106 | C | C | | |
| CPD-107 | C | C | | |
| CPD-108 | C | C | | |
| CPD-109 | B | A | | |
| CPD-110 | A | A | | |
| CPD-111 | B | B | | |
| CDP-112 | | | A | A |
| CPD-113 | | | B | A |
| CPD-114 | | | A | A |
| CPD-115 | | | A | A |
| CPD-116 | | | C | A |
| CPD-117 | | | C | A |
| CPD-118 | | | B | A |
| CPD-119 | | | | A |
| CPD-120 | | | | A |
| CPD-121 | | | A | A |
| CPD-122 | | | | C |
| CPD-123 | | | B | A |
| CPD-124 | | | A | A |
| CPD-125 | | | A | A |
| CPD-126 | | | A | A |
| CPD-127 | | | A | A |
| CPD-128 | | | | C |
| CPD-129 | | | C | A |
| CPD-130 | | | | B |
| CPD-131 | | | A | A |
| CPD-132 | | | | A |
| CPD-133 | | | A | A |
| CPD-134 | | | A | A |
| CPD-135 | | | B | A |
| CPD-136 | | | B | A |
| CPD-137 | | | C | A |
| CPD-138 | | | | C |
| CPD-139 | | | C | A |
| CPD-140 | | | C | A |
| CPD-141 | | | B | A |
| CPD-142 | | | B | A |
| CPD-143 | | | A | A |
| CPD-144 | | | A | A |
| CPD-145 | | | | B |
| CPD-146 | | | B | A |
| CPD-147 | | | B | A |
| CPD-148 | | | A | A |
| CPD-149 | | | | A |
| CPD-150 | | | A | A |
| CPD-151 | | | A | A |
| CPD-152 | | | | A |
| CPD-153 | | | | C |
| CPD-154 | | | | B |
| CPD-155 | | | A | A |
| CPD-156 | | | | B |
| CPD-157 | | | A | C |
| CPD-158 | | | A | B |
| CPD-159 | | | A | A |
| CPD-160 | | | | C |
| CPD-161 | | | C | A |
| CPD-162 | | | B | A |
| CPD-163 | | | | C |
| CPD-164 | | | A | B |
| CPD-165 | | | C | C |
| CPD-166 | | | | C |
| CPD-167 | | | A | C |
| CPD-168 | | | | C |
| CDD-169 | | | C | C |
| CDD-170 | | | B | A |
| CDD-171 | | | B | B |
| CDD-172 | | | C | C |
| CDD-173 | | | C | C |
| CDD-174 | | | C | C |
| CDD-175 | | | A | A |
| CDD-176 | | | C | C |

The percentage of TYK2 protein degradation of each compound at 0.5 and 5 μM or at 10 and 100 nM was determined in MOLT-4 or NOMO-1 cells respectively as described in Methods. A: ≥60% TYK2 degradation; B: <60% TYK2 degradation, and >20% TYK2 degradation; C: ≤20% TYK2 degradation.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-88. (canceled)

89. A heterobifunctional compound of FORMULA I: 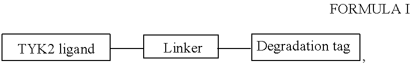
or a pharmaceutically acceptable salt thereof,
wherein:
1) the TYK2 ligand comprises a moiety of FORMULA 1-1E: 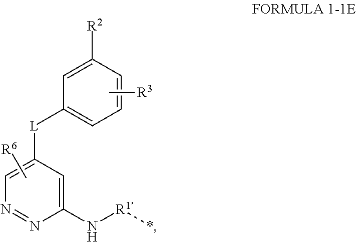
wherein
* indicates the connection to the linker moiety of the heterobifunctional compound;
L is selected from CR⁴R⁵, N⁴, and O;
R⁴ and R⁵ are independently selected from the group consisting of H, optionally substituted C₁-C₆ alkyl, and optionally substituted C₃-C₆ cycloalkyl;
R⁶, at each occurrence, is independently selected from the group consisting of hydrogen, halogen, CN, NO₂, COR⁷, CON(R⁷)R⁸, optionally substituted C₁-C₈ alkyl, optionally substituted C₂-C₈ alkenyl, optionally substituted C₃-C₁₀ carbocyclyl, optionally substituted 3-10 membered heterocyclyl;
R⁷ and R⁸ are independently selected from the group consisting of hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted C₃-C₁₀ carbocyclylC₁-C₈alkyl, optionally substituted 3-10 membered heterocyclylC₁-C₈alkyl, optionally substituted C₃-C₁₀ carbocyclyl, optionally substituted 3-10 membered heterocyclyl, optionally substituted aryl, and optionally substituted heteroaryl, or R⁷ and R⁸-together with the atom to which they are connected form a 3-20 membered heterocyclyl ring;
R² is selected from H, CN, halogen, CO₂R¹⁰, CONR¹⁰R¹¹, optionally substituted aryl, and optionally substituted heteroaryl;
R¹⁰ and R¹¹ are independently selected from the group consisting of hydrogen, optionally substituted C₁-C₈ alkyl, optionally substituted C₂-C₈ alkenyl, optionally substituted C₂-C₈ alkynyl, optionally substituted C₃-C₁₀ carbocyclyl, optionally substituted 3-10 membered heterocyclyl, optionally substituted aryl, and optionally substituted heteroaryl; or R¹⁰ and R¹¹, together with the atom(s) to which they are connected optionally form an optionally substituted 3-20 membered heterocyclyl ring;
R^{1′} is a divalent group independently selected from the group consisting of null, R′—R″, R′OR″, R′SR″, R′N(R¹³)R″, R′OC(O)R″, R′OC(O)OR″, R′OCON(R¹³)R″, R′C(O)R″, R′C(O)OR″, R′CON(R¹³)R″, R′S(O)R″, R′S(O)₂R″, R′SO₂N(R¹³)R″, R′NR¹⁴C(O)OR″, R′NR¹⁴C(O)R″, R′NR¹⁴C(O)N(R¹³)R″, R′NR¹⁴S(O)R″, R′NR¹⁴S(O)₂R″, and R′NR¹⁴S(O)₂NR¹³R″, optionally substituted C₃-C₁₃ carbocyclyl, optionally substituted 3-13 membered heterocyclyl, optionally substituted aryl, and optionally substituted heteroaryl, wherein
R′ and R″ are divalent groups independently selected from the group consisting of null, optionally substituted C₁-C₈ alkylene, optionally substituted C₂-C₈ alkenylene, optionally substituted C₂-C₈ alkynylene, optionally substituted C₃-C₁₀ carbocyclyl, optionally substituted 3-10 membered heterocyclyl, optionally substituted aryl, and optionally substituted heteroaryl;
R¹³ and R¹⁴ are independently selected from the group consisting of H, optionally substituted C₁-C₈ alkyl, optionally substituted C₂-C₈alkenyl, optionally substituted C₂-C₈ alkynyl, optionally substituted C₃-C₁₀ carbocyclyl, optionally substituted 3-10 membered heterocyclyl, optionally substituted aryl, and optionally substituted heteroaryl;
R³ means one or more substituents which are selected from the group consisting of null, hydrogen, halogen, CN, NO₂, OR¹⁵, SR¹⁵, NR¹⁵R¹⁶, OCOR¹⁵, OCO₂R¹⁵, OCON(R¹⁵)R¹⁶, COR¹⁵, CO₂R¹⁵, CON(R¹⁵)R¹⁶, SOR¹⁵, SO₂R¹⁵, SO₂N(R¹⁵)R¹⁶, NR¹⁷CO₂R¹⁵, NR¹⁷COR¹⁵, NR¹⁷C(O)N(R¹⁵)R¹⁶, NR¹⁷SOR¹⁵, NR¹⁷SO₂R¹⁵, NR¹⁷SO₂N(R¹⁵)R¹⁶, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered heterocyclylC₁-C₈alkyl, optionally substituted C₃-C₁₀ carbocyclylC₁-C₈alkyl, optionally substituted C₃-C₁₀ carbocyclyl, optionally substituted 3-10 membered heterocyclyl, optionally substituted aryl, and optionally substituted heteroaryl; or two R³ groups together with the atoms to which they are connected optionally form optionally substituted C₅-C₆ carbocyclyl, optionally substituted 5-6 membered heterocyclyl, optionally substituted C₆ aryl, and optionally substituted 5-6 membered heteroaryl, wherein
R¹⁵, R¹⁶, and R¹⁷ are independently selected from the group consisting of null, hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted C₃-C₁₀ carbocyclylC₁-C₈alkyl, optionally substituted 3-10 membered heterocyclylC₁-C₈alkyl, optionally substituted C₃-C₁₀ carbocyclyl, optionally substituted 3-10 membered heterocyclyl, optionally substituted aryl, and optionally substituted heteroaryl, or R¹⁵ and R¹⁶, or R¹⁵ and R¹⁷ together with the atom to which they are connected form a 3-20 membered heterocyclyl ring;
2) the degradation tag is a moiety of FORMULA 6A, 6B, or 6C: 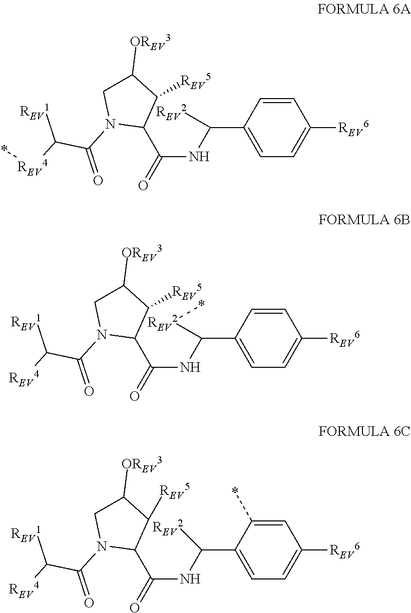
wherein * indicates the connection to the linker moiety of the heterobifunctional compound;
R_{EV}¹ and R_{EV}² are independently selected from the group consisting of hydrogen, hydroxyl, amino, cyano, nitro, optionally substituted C₁-C₈ alkyl, optionally substituted C₂-C₈ alkenyl, optionally substituted C₂-C₈ alkynyl; optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted C₃-C₁₀ carbocyclyl, optionally substituted 3-10 membered heterocyclyl;
R_{EV}^{2′} is a divalent group selected from the group consisting of null, O, NH, optionally substituted C₁-C₈ alkylene, optionally substituted C₂-C₈ alkenylene, optionally substituted C₂-C₈ alkynylene; optionally substituted C₁-C₈alkoxyC₁-C₈alkylene, optionally substituted C₁-C₈ haloalkylene, optionally substituted C₁-C₈ hydroxyalkylene, optionally substituted C₁-C₈ aminoalkylene, optionally substituted C₁-C₈alkylaminoC₁-C₈alkylene, optionally substituted C₃-C₁₀ carbocyclyl, and optionally substituted 3-10 membered heterocyclyl;
R_{EV}³ is selected from the group consisting of hydrogen, optionally substituted —C(O)R_{EV}⁷, —C(O)OR_{EV}⁷, —C(O)NR_{EV}⁷R_{EV}⁸, —P(O)(OR_{EV}⁷)₂, and —CR_{EV}⁷R_{EV}⁸—OP(O)(OR_{EV}⁹)₂, wherein
R_{EV}⁷, R_{EV}⁸ and R_{EV}⁹ are independently selected from the group consisting of hydrogen, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted C₃-C₁₀ carbocyclyl, optionally substituted 3-10 membered heterocyclyl, optionally substituted C₂-C₈ alkenyl, optionally substituted C₂-C₈ alkynyl, optionally substituted aryl and optionally substituted heteroaryl;
R_{EV}⁴ is selected from the group consisting of —N(R_{EV}¹⁰)R_{EV}¹¹, —OR_{EV}¹⁰, —N(R_{EV}¹⁰)C(O)R_{EV}¹¹, optionally substituted C₃-C₁₀ carbocyclyl, optionally substituted 3-10 membered heterocyclyl, optionally substituted aryl, and optionally substituted heteroaryl;
R_{EV}^{4′} is a divalent group selected from the group consisting of —N(R_{EV}¹⁰)—, —O—, —N(R_{EV}¹⁰)C(O)R_{EV}^{11′}—, optionally substituted C₃-C₁₀ carbocyclyl, optionally substituted 3-10 membered heterocyclyl, optionally substituted aryl, and optionally substituted heteroaryl;
R_{EV}¹⁰ is selected from the group consisting of hydrogen, optionally substituted C₁-C₈alkyl, optionally substituted C₃-C₈cycloalkyl, optionally substituted C₁-C₈alkyl-CO, optionally substituted C₁-C₈cycloalkyl-CO, optionally substituted C₃-C₈cycloalkyl-C₁-C₈alkyl-CO, optionally substituted 3-10 membered heterocyclyl-CO, optionally substituted 3-10 membered hetercyclyl-C₁-C₈alkyl-CO, optionally substituted aryl-CO, optionally substituted aryl-C₁-C₈alkyl-CO, optionally substituted heteroaryl-CO, optionally substituted heteroaryl-C₁-C₈alkyl-CO, optionally substituted aryl, and optionally substituted heteroaryl;
R_{EV}¹¹ is selected from the group consisting of hydrogen, optionally substituted C₁-C₈alkyl, optionally substituted C₃-C₈cycloalkyl, optionally substituted 3-8 membered heterocycloalkyl, optionally substituted C₃-C₈ carbocyclclyl, and optionally substituted C₃-C₈ heterocyclclyl;
R_{EV}^{11′} at each occurrence, is a divalent group independently selected from the group consisting of null, O, optionally substituted C₁-C₈alkylene, optionally substituted C₃-C₈ cycloalkylene, optionally substituted 3-8 membered heterocycloalkylene, optionally substituted C₃-C₈ carbocyclclyl, and optionally substituted C₃-C₈ heterocyclclyl;
R_{EV}⁵ is selected from the group consisting of hydrogen and halogen (such as F); and
R_{EV}⁶ is selected from hydrogen, halogen, hydroxyl, amino, cyano, nitro, optionally substituted C₁-C₈alkyl, optionally substituted C₁-C₈cycloalkyl, optionally substituted C₁-C₈alkoxy, and optionally substituted C₁-C₈cycloalkoxy, optionally substituted C₁-C₈heterocycloalkyl, optionally substituted aryl, and optionally substituted heteroaryl;
or
the degradation tag is a moiety of FORMULA 5, and the degradation tag is connected to the linker moiety of the heterobifunctional compound via Z_{E}; 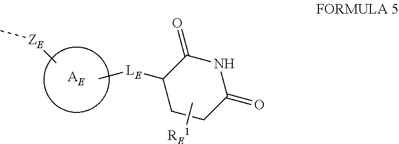
wherein
Z_{E} is a divalent group of —(R_{E}^{z})_{nE}—; wherein Subscript n_{E}=0, 1, 2, 3, 4, 5 or 6; wherein R_{E}^{Z}, at each occurrence, is independently R_{E}^{r}, or R_{E}^{w}; wherein R_{E}^{w}, at each occurrence, is a bond or selected from the group consisting of —CO—, —CR_{E}⁵R_{E}⁶—, —NR_{E}⁵—, —O—, —S—, —S(O)—, —S(O)₂—, —C≡C—, optionally substituted C₁-C₁₀ alkylene, optionally substituted C₂-C₁₀ alkenylene, optionally substituted C₂-C₁₀ alkynylene; and R_{E}^{r}, at each occurrence, is a bond, or selected from the group consisting of optionally substituted C₃-C₁₀ carbocyclyl such as 3-13 membered carbocyclyl, optionally substituted 3-13 membered heterocyclyl, optionally substituted aryl, and optionally substituted heteroaryl; with the proviso that —R_{E}^{z}—R_{E}^{z}— is not —O—O—;
R_{E}⁵ and R_{E}⁶, at each occurrence, are independently selected from the group consisting of hydrogen, halogen, oxo, hydroxy, amino, cyano, nitro, optionally substituted C₁-C₆ alkyl, optionally substituted C₁-C₈ alkoxy, optionally substituted C₁-C₈ alkylamino, optionally substituted 3 to 8 membered carbocyclyl, and optionally substituted 3 to 8 membered heterocyclyl; or R_{E}⁵ and R_{E}⁶ together with the atom(s) to which they are connected optionally form an optionally substituted 3-8 membered cycloalkyl or an optionally substituted heterocyclyl;
R_{E}¹ is selected from the group consisting of hydrogen, halogen, cyano, nitro, optionally substituted C₁-C₆ alkyl, optionally substituted C₁-C₈ heteroalkyl, optionally substituted 3-8 membered carbocyclyl, and optionally substituted 3-8 membered heterocyclyl;
L_{E} is a divalent group selected from the group consisting of null, -L_{E}¹-, and -L_{E}¹-L_{E}²-; wherein L_{E}¹ and L_{E}² are independently selected from the group consisting of —CO—, —O—, —CR_{E}¹⁰R_{E}¹¹— and —NR_{E}¹⁰—, with the proviso that -L_{E}¹-L_{E}²- is not —O—O—; wherein R_{E}¹⁰ and R_{E}¹¹ are independently selected from the group consisting of hydrogen, halogen, cyano, nitro, hydroxy, amino, optionally substituted C₁-C₆ alkyl, optionally substituted C₁-C₆ alkoxy, and optionally substituted C₁-C₆ alkylamino;
Ring A_{E} is a divalent group selected from the group consisting of FORMULAE A_{E}1, A_{E}2, A_{E}3, A_{E}4, A_{E}5, A_{E}6 and A_{E}7: 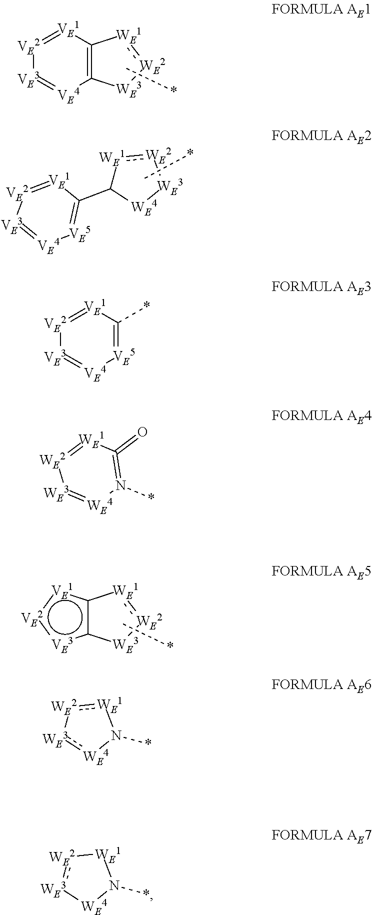
wherein
* indicates the attachment to L_{E}, and Z_{E} is attached to any possible position on the Ping A_{E}; [symbol]
indicates a single bond or a double bond;
V_{E}¹, V_{E}², V_{E}³, V_{E}⁴ and V_{E}⁵, at each occurrence, are each independently selected from the group consisting of a bond, C, CR_{E}⁵, S, N, and NR_{E}²; or V_{E}¹ and V_{E}², V_{E}² and V_{E}³, V_{E}³ and V_{E}⁴, or V_{E}⁴ and V_{E}⁵ are combined together to optionally form Co aryl ring or a 5, 6 or 7 membered heteroaryl ring;
R_{E}², at each occurrence, is independently selected from the group consisting of absent, hydrogen, halogen, cyano, nitro, hydroxy, amino, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, optionally substituted C₁-C₆ alkoxy, optionally substituted C₁-C₆ alkylamino, optionally substituted 3-8 membered carbocyclyl, and optionally substituted 3-8 membered heterocyclyl; or R_{E}² and another RU together with the atom(s) to which they are connected form optionally substituted 3-8 membered cycloalkyl, optionally substituted 3-8 membered heterocyclyl, optionally substituted aryl, and optionally substituted heteroaryl;
W_{E}¹, W_{E}², W_{E}³ and W_{E}⁴ are each independently selected from the group consisting of —N═, —C≡, —CR_{E}³═, —CO—, —O—, —CR_{E}³R_{E}⁴—, —NR_{E}³—, —CR_{E}³═CR_{E}⁴—, —N═CR_{E}³—, and —N═N—; or W_{E}¹ and W_{E}², W_{E}² and W_{E}³, or W_{E}³ and W_{E}⁴ are combined together to optionally form optionally substituted C₆ aryl or optionally substituted 5-, 6- or 7-membered heteroaryl;
R_{E}³ and R_{E}⁴, at each occurrence, are independently selected from the group consisting of absent, hydrogen, halogen, cyano, nitro, hydroxy, amino, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, optionally substituted C₁-C₆ alkoxy, optionally substituted C₁-C₆ alkylamino, optionally substituted arylamino, optionally substituted heteroarylamino, optionally substituted 3 to 8 membered carbocyclyl, and optionally substituted 3 to 8 membered heterocyclyl; or R_{E}³ and R_{E}⁴, on the same atom or on the adjacent atoms, together with the atom(s) to which they are connected optionally form an optionally substituted 3-8 membered cycloalkyl or heterocyclyl ring, optionally substituted aryl, and optionally substituted heteroaryl;
and 3) the linker moiety is of FORMULA 9: 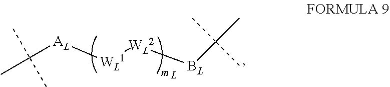
wherein
A_{L}, W_{L}¹, W_{L}², and B_{L}, at each occurrence, are bivalent moieties independently selected from the group consisting of null, R_{L}^{d}—R_{L}^{e}, R_{L}^{d}COR_{L}^{e}, R_{L}^{d}C(O)OR_{L}^{e}, R_{L}^{d}C(O)N(R_{L}¹)R_{L}^{e}, R_{L}^{d}C(S)N(R_{L}¹)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}¹)R_{L}^{e}, R_{L}^{d}N(R_{L}¹)R_{L}^{e}, R_{L}^{d}N(R_{L}¹)COR_{L}^{e}, R_{L}^{d}N(R_{L}¹)CON(R_{L}²)R_{L}^{e}, R_{L}^{d}N(R_{L}¹)C(S)R_{L}^{e}, optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ cycloalkyl, optionally substituted 3-13 membered heterocyclyl, optionally substituted aryl, and optionally substituted heteroaryl, wherein
R_{L}^{d} and R_{L}^{e}, at each occurrence, are independently selected from null, R_{L}^{r}, optionally substituted (C₁-C₈ alkylene)-R_{L}^{r}, optionally substituted R_{L}^{r}—(C₁-C₈ alkylene), optionally substituted (C₁-C₈ alkylene)-R_{L}^{r}-(C₁-C₈ alkylene), or a bivalent moiety comprising of optionally substituted C₁-C₈ alkylene, optionally substituted C₁-C₈ alkenylene, optionally substituted C₂-C₈ alkynylene, optionally substituted C₁-C₈ heteroalkylene, optionally substituted C₁-C₈ heteroalkenylene, optionally substituted C₂-C₈ heteroalkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted C₃-C₁₃ cycloalkyl, optionally substituted 3-13 membered heterocyclyl, optionally substituted aryl, and optionally substituted heteroaryl;
R_{L}^{r}, at each occurrence, is selected from optionally substituted C₃-C₁₀ carbocyclyl, optionally substituted 3-10 membered heterocyclyl, optionally substituted aryl, and optionally substituted heteroaryl;
R_{L}¹ and R_{L}², at each occurrence, are independently selected from the group consisting of hydrogen, optionally substituted C₁-C₈ alkyl, optionally substituted C₂-C₈alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₀ carbocyclyl, optionally substituted 3-10 membered heterocyclyl, optionally substituted aryl, and optionally substituted heteroaryl;
R_{L}^{d} and R_{L}^{e}, R_{L}¹ and R_{L}², R_{L}^{d} and R_{L}¹, R_{L}^{d} and R_{L}², R_{L}^{e} and R_{L}¹, or R_{L}^{e} and R_{L}² together with the atom(s) to which they are connected optionally form a C₃-C₂₀ carbocyclyl or 3-20 membered heterocyclyl ring; and
m_{L} is 1, 2, 3, 4, 5, 6, 7, 8, 9, 10, 11, 12, 13, 14, or 15.

90. The heterobifunctional compound of claim 89, or a pharmaceutically acceptable salt thereof, wherein the degradation tag is a moiety of FORMULA 6A, 613, or 6C.

91. The heterobifunctional compound of claim 89, or a pharmaceutically acceptable salt thereof, wherein L is selected from NH and N(CH₃).

92. The heterobifunctional compound of claim 89, or a pharmaceutically acceptable salt thereof, wherein the TYK2 ligand is a moiety of FORMULA 1-1G:

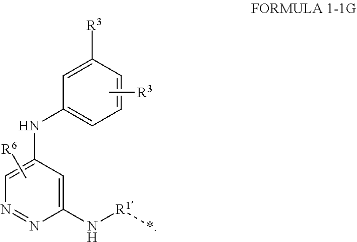

93. The heterobifunctional compound of claim 89, or a pharmaceutically acceptable salt thereof, wherein the TYK2 ligand is a moiety of FORMULA 1-1I: 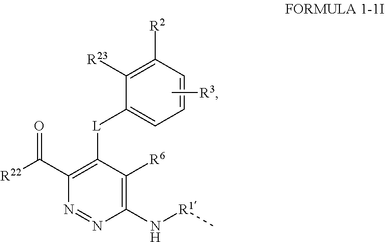
wherein R²² is R⁷ or NHR⁷; and R²³ is R³.

94. The heterobifunctional compound of claim 89, or a pharmaceutically acceptable salt thereof, wherein R² is selected from H, CN, F, Cl, Br, CO₂H, CONH₂, CONHCH₃, optionally substituted triazolyl and optionally substituted phenyl.

95. The heterobifunctional compound of claim 89, or a pharmaceutically acceptable salt thereof, wherein R² is selected from optionally substituted triazolyl and optionally substituted phenyl.

96. The heterobifunctional compound of claim 89, or a pharmaceutically acceptable salt thereof, wherein R^{1′} is a divalent group selected from the group consisting of null, R′—R″, R′C(O)R″, optionally substituted C₃-C₁₃ carbocyclyl, optionally substituted 3-13 membered heterocyclyl, optionally substituted aryl, and optionally substituted heteroaryl; and R′ and R″ are divalent groups independently selected from the group consisting of null, optionally substituted C₂-C₈ alkynylene, optionally substituted C₃-C₁₀ carbocyclyl, optionally substituted 3-10 membered heterocyclyl, optionally substituted aryl, and optionally substituted heteroaryl.

97. The heterobifunctional compound of claim 89, or a pharmaceutically acceptable salt thereof, wherein R_{1′} is a bivalent group selected from optionally substituted C₁-C₈ alkylene, optionally substituted C₃-C₁₃ carbocyclyl, optionally substituted 3-13 membered heterocyclyl, optionally substituted aryl, and optionally substituted heteroaryl.

98. The heterobifunctional compound of claim 89, or a pharmaceutically acceptable salt thereof, wherein R^{1′} is selected from C(O), optionally substituted C(O)—CH₂, and optionally substituted pyridinyl.

99. The heterobifunctional compound of claim 93, or a pharmaceutically acceptable salt thereof, wherein R² is selected from NH₂, NHMe, NHCD₃, Me, Et, CD₃, CH₂CD₃, iPr, and cPr.

100. The heterobifunctional compound of claim 93, or a pharmaceutically acceptable salt thereof, wherein R²³ is selected from H, F, OMe, CONH₂, CONH Me, SMe, SOMe, SO₂Me, OCD₃, CONHCD₃, SCD₃, SOCD₃, and SO₂CD₃.

101. The heterobifunctional compound of claim 89, or a pharmaceutically acceptable salt thereof, wherein the Degradation tag is: (i) a moiety of FORMULA 6A-1, 6B-1, 6C-1, 6A-2, 6B-2, or 6C-2: 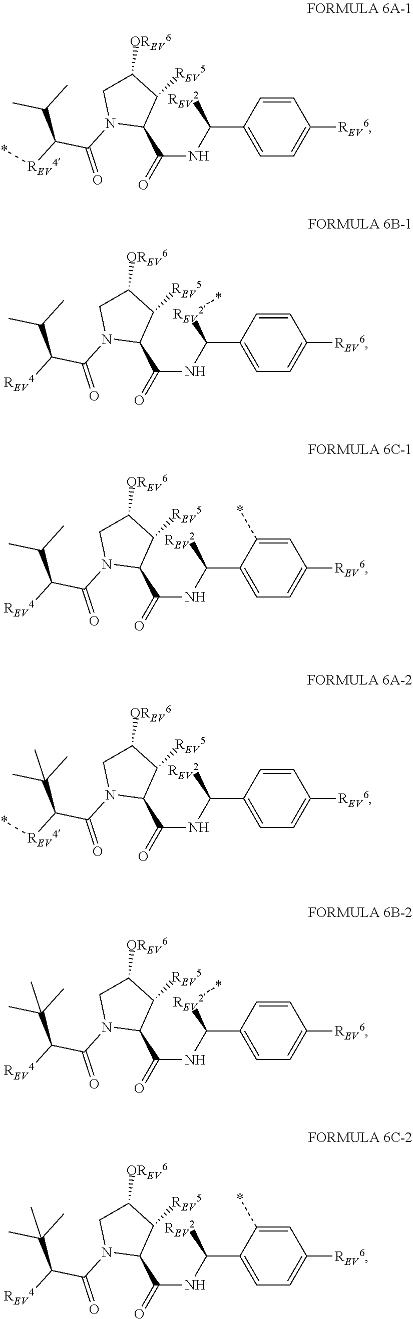
wherein R_{EV}², R_{EV}^{2′}, R_{EV}³, R_{EV}⁴, R_{EV}^{4′}, R_{EV}⁵, and R_{EV}⁶ are defined as in FORMULAE 6A, 6B, and 6C;
(ii) a moiety of FORMULA 6A-3, 6B-3, 6C-3, 6A-4, 6B-4, or 6C-4: 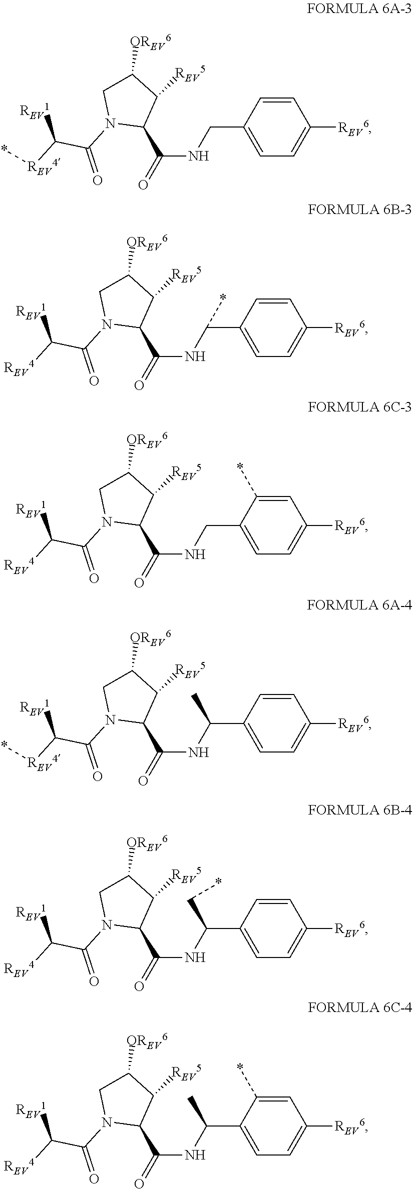
wherein R_{EV}¹, R_{EV}³, R_{EV}⁴, R_{EV}^{4′}, R_{EV}⁵, and R_{EV}⁶ are defined as in FORMULAE 6A, 6B, and 6C; or
(iii) a moiety of FORMULA 6A-5, 6B-5, or 6C-5: 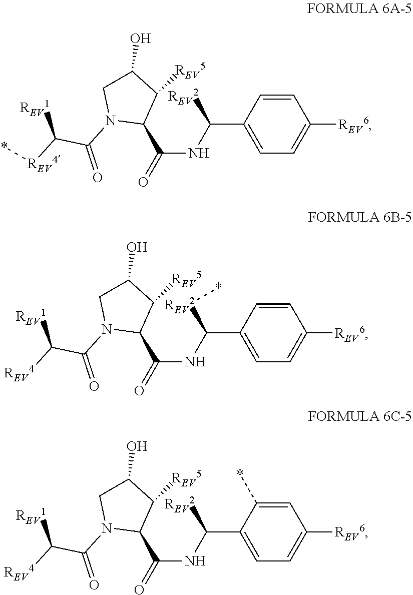
wherein R_{EV}¹, R_{EV}², R_{EV}^{2′}, R_{EV}⁴, R_{EV}^{4′}, R_{EV}⁵, and R_{EV}⁶ are defined as in FORMULAE 6A, 6B, and 6C.

102. The heterobifunctional compound of claim 89, or a pharmaceutically acceptable salt thereof, wherein the Degradation tag is: (i) a moiety of FORMULA 6A-6, 6B-6, 6C-6, 6A-7, 6B-7, or 6C-7: 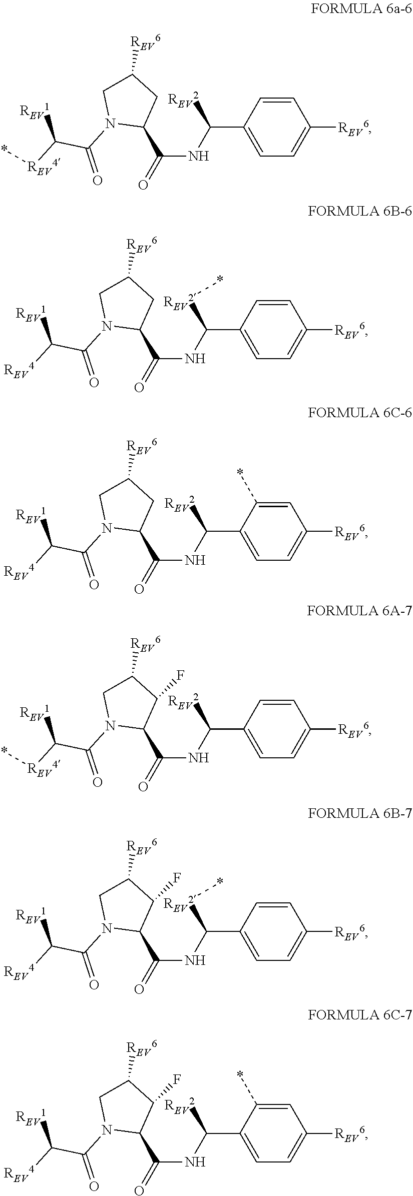
wherein R_{EV}¹, R_{EV}², R_{EV}^{2′}, R_{EV}³, R_{EV}⁴, R_{EV}^{4′}, and R_{EV}⁶ are defined as in FORMULAE 6A, 6B, and 6C; or
(ii) a moiety of FORMULA 6A-8, 6B-8, or 6C-8: 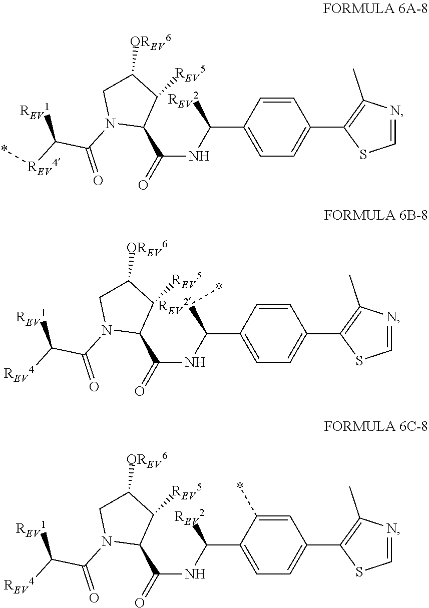
wherein R_{EV}¹, R_{EV}², R_{EV}^{2′}, R_{EV}³, R_{EV}⁴, R_{EV}^{4′}, and R_{EV}⁵ are defined as in FORMULAE 6A, 6B, and 6C.

103. The heterobifunctional compound of claim 89, or a pharmaceutically acceptable salt thereof, wherein R_{EV}⁴ is selected from NH₂, NHC(O)Me, 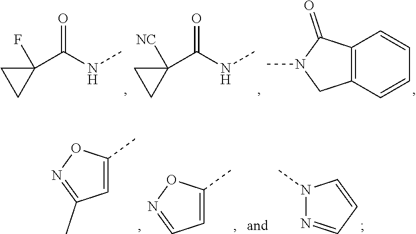
and/or R_{EV}^{4′} is selected from NH, C(O)NH, CH₂C(O)NH, 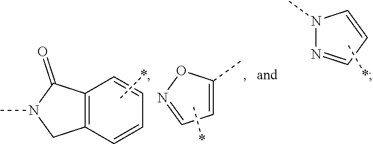
wherein * indicates the connection to the linker moiety of the heterobifunctional compound.

104. The heterobifunctional compound of claim 89, or a pharmaceutically acceptable salt thereof, wherein the Degradation tag is a moiety of FORMULA 6A-9, 6A-10, 6A-11, 6A-12, 6A-13, 6B-9, 6B-10, 6B-11, 6B-12, 6B-13, 6B-14, 6B-15, 6C-9, 6C-10, 6C-11, 6C-12, 6C-13, 6C-14, or 6C-15: 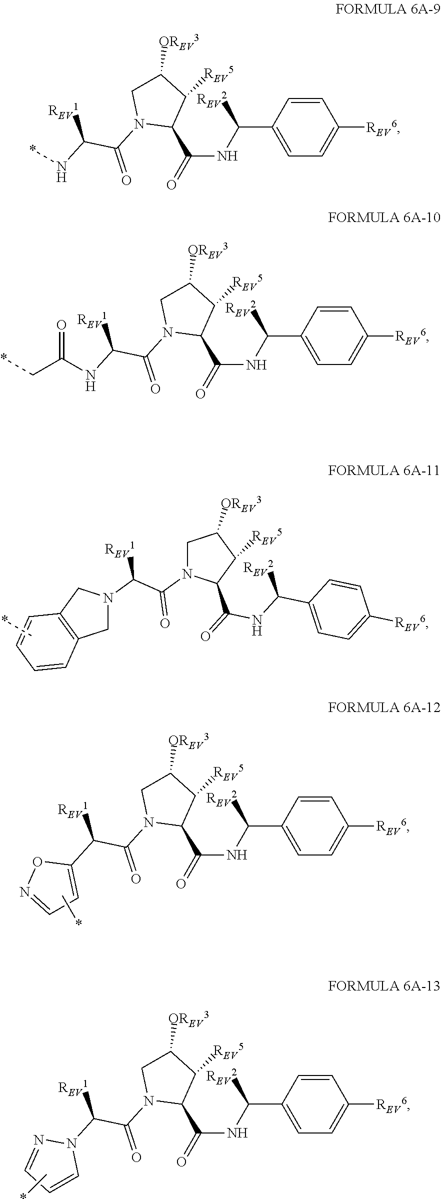 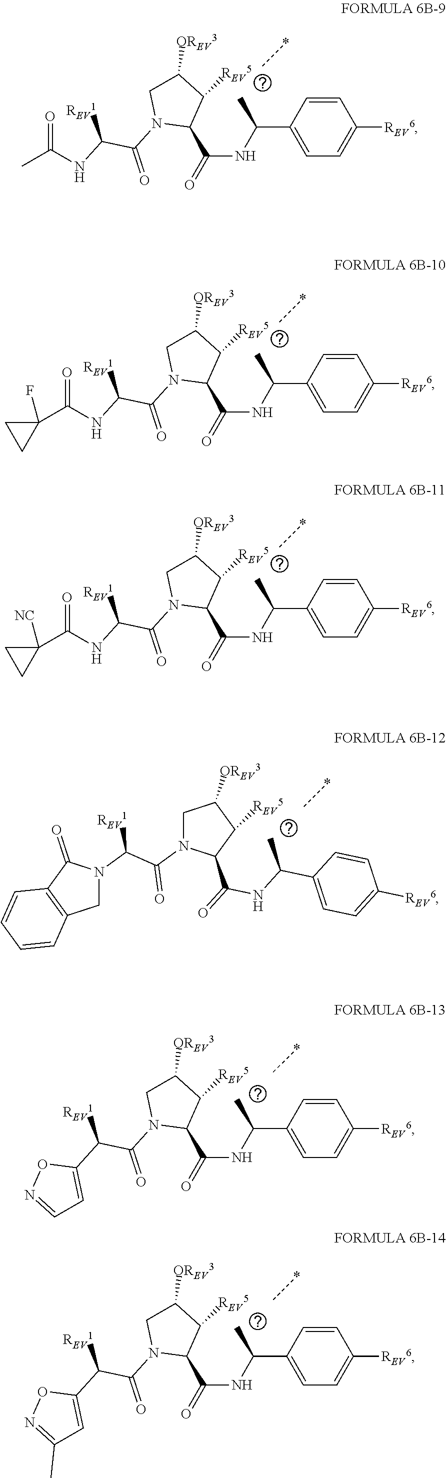 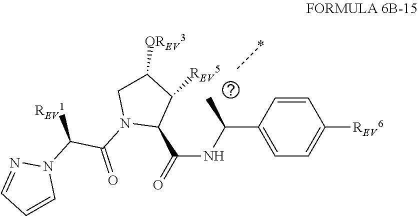 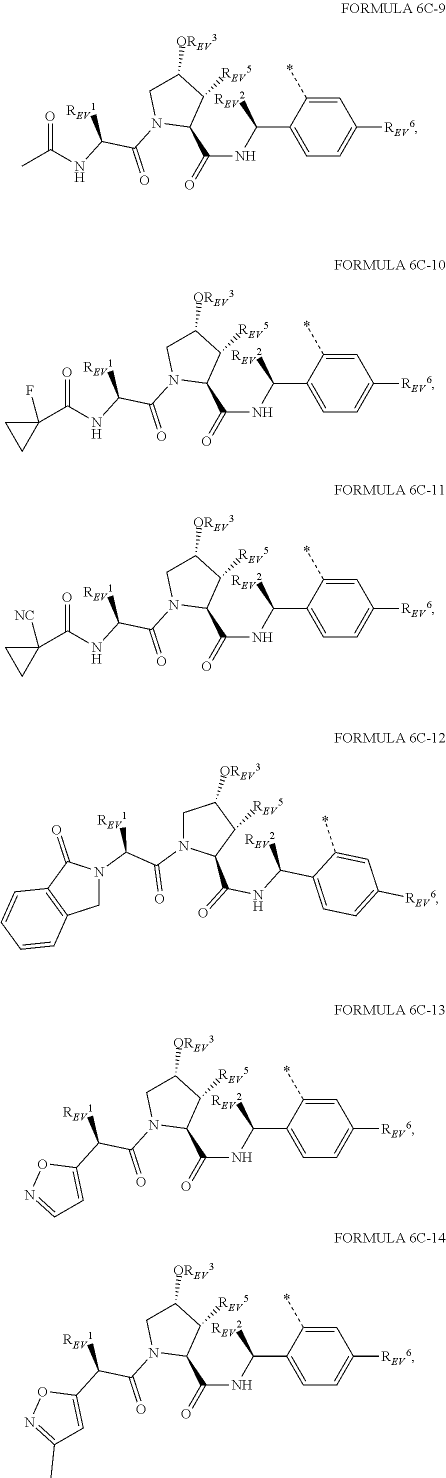 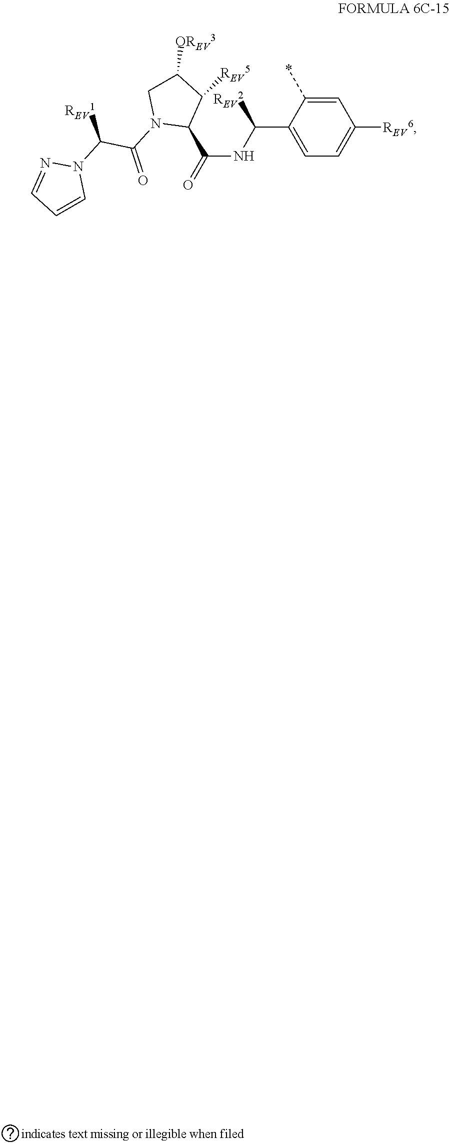
wherein R_{EV}¹, R_{EV}², R_{EV}^{2′}, R_{EV}³, R_{EV}⁵, and R_{EV}⁶ are defined as in FORMULAE 6A, 6B, and 6C.

105. The heterobifunctional compound of claim 89, or a pharmaceutically acceptable salt thereof, wherein the Degradation tag is a moiety of any of FORMULAE 7A to 7BJ:

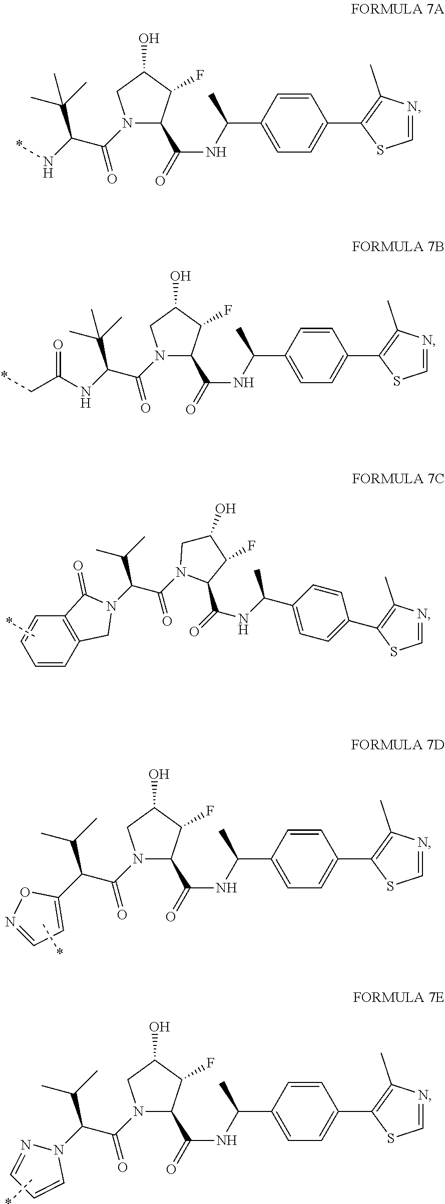

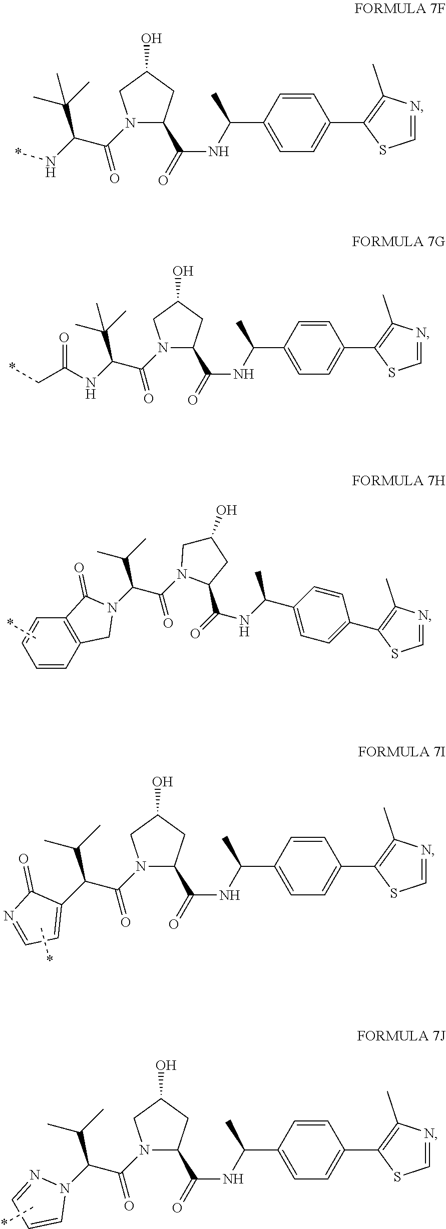

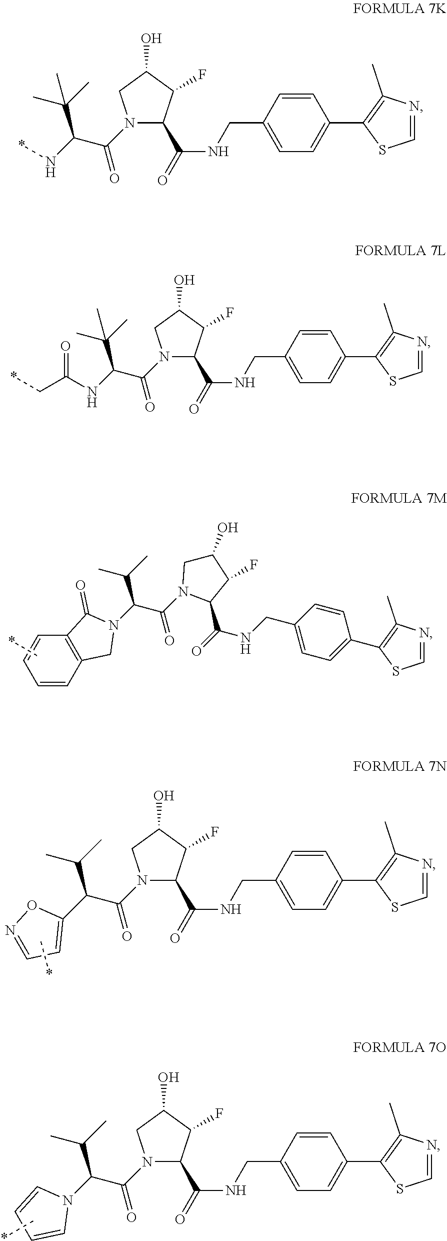

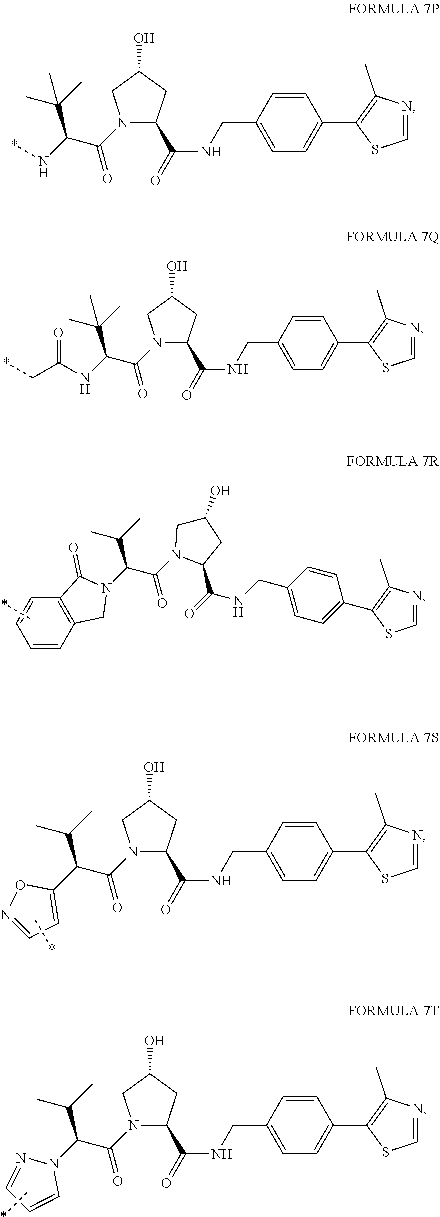

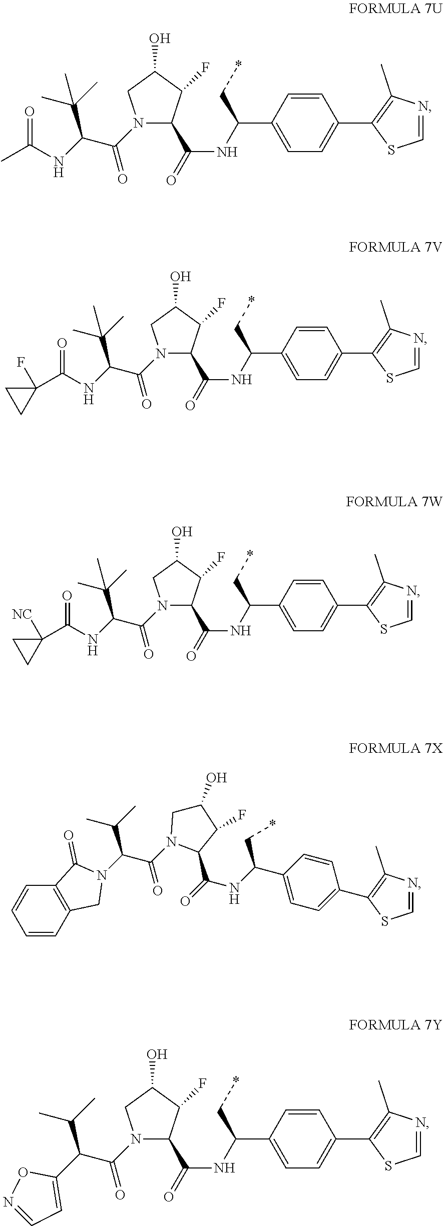

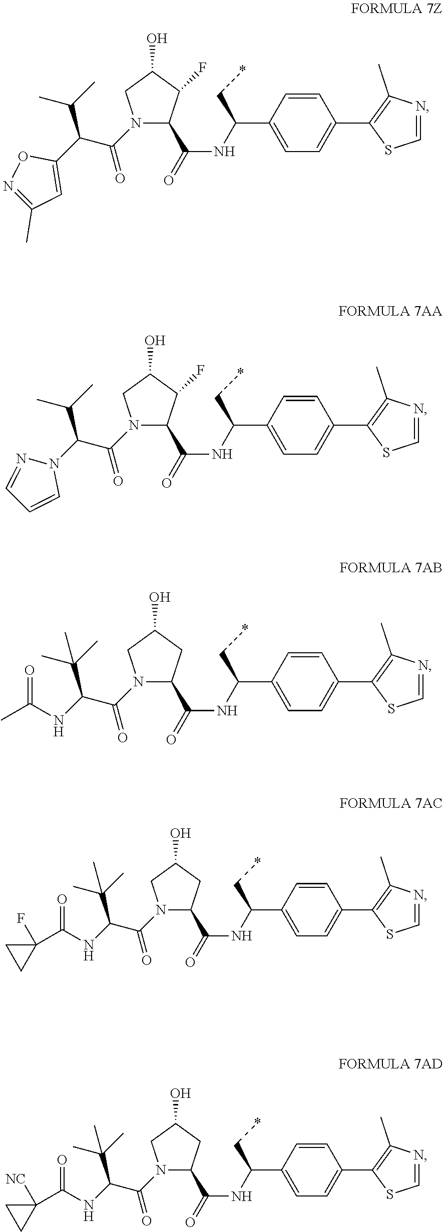

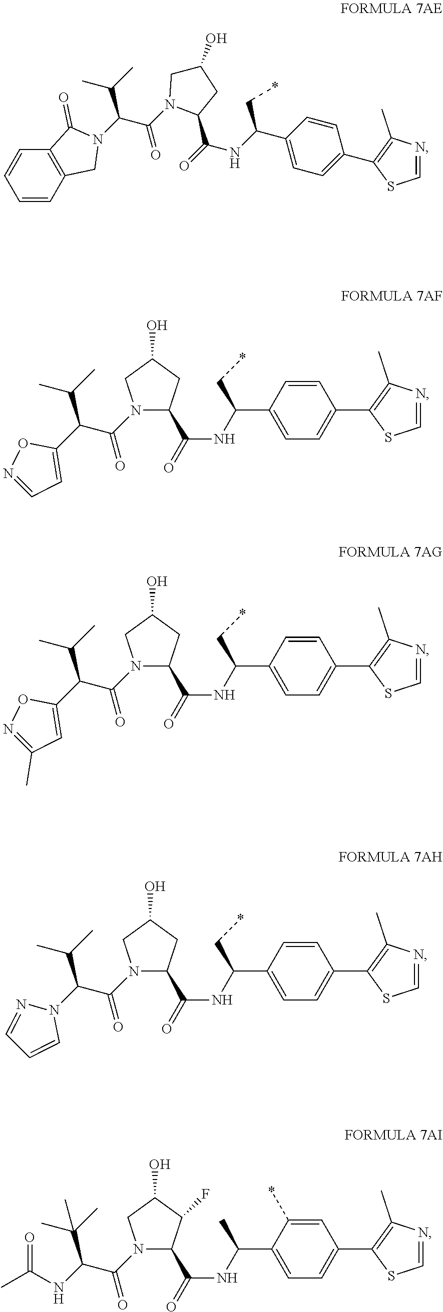

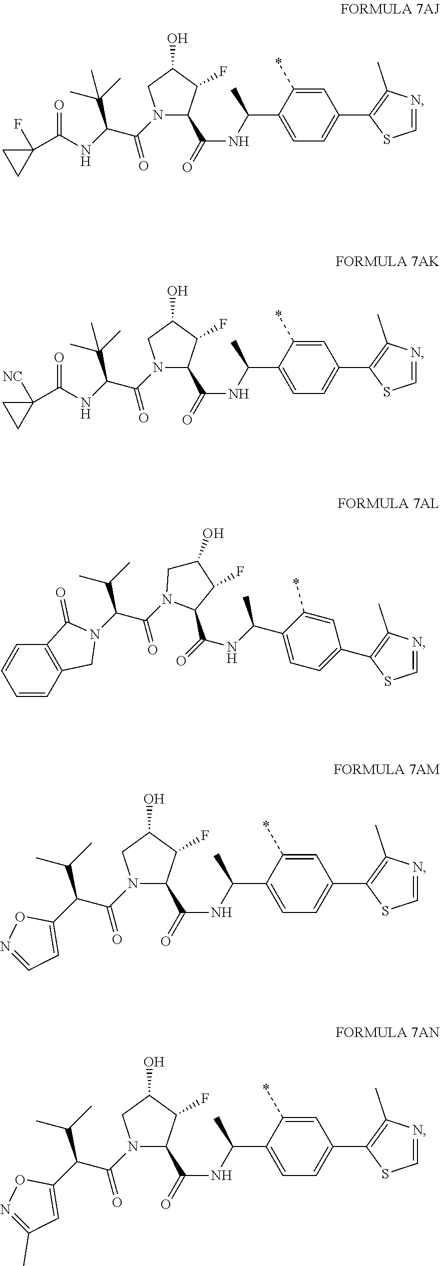

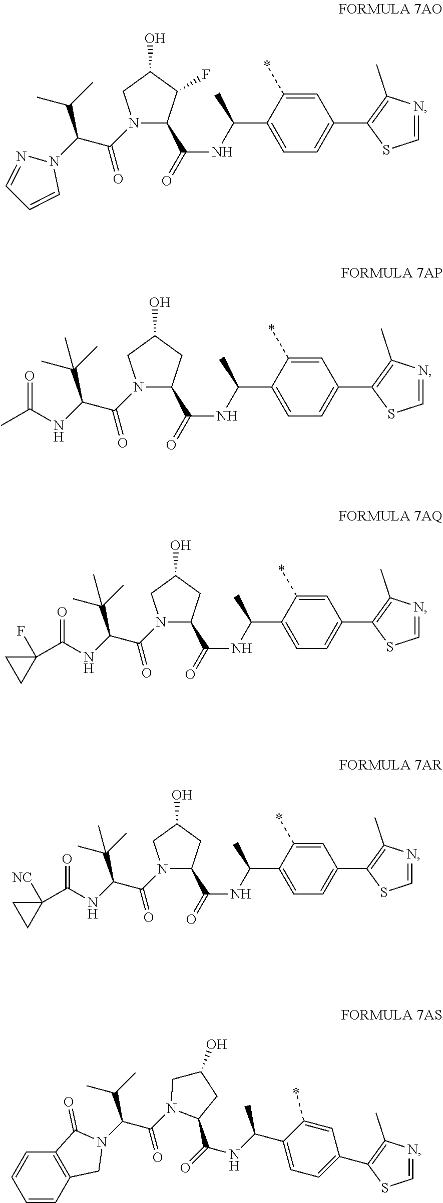

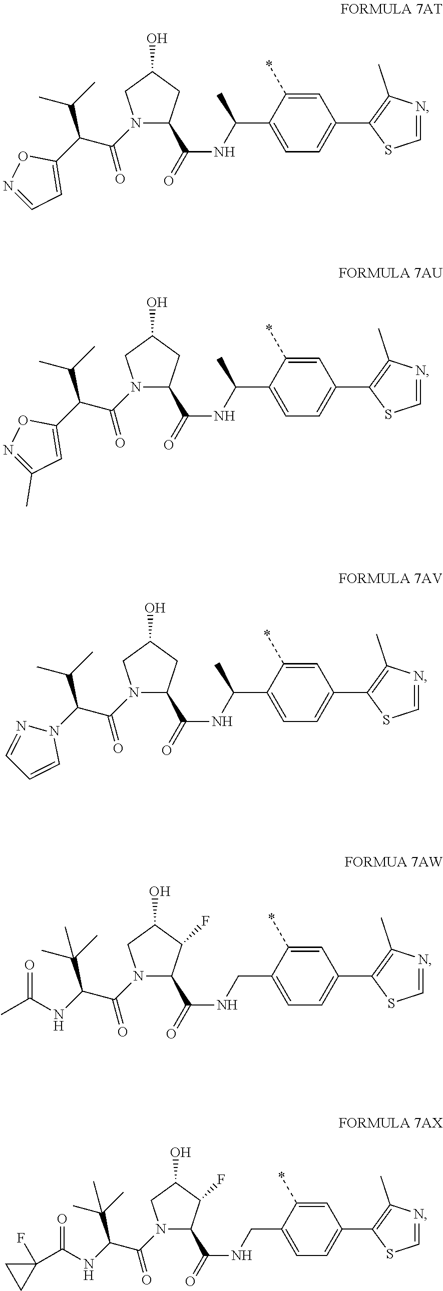

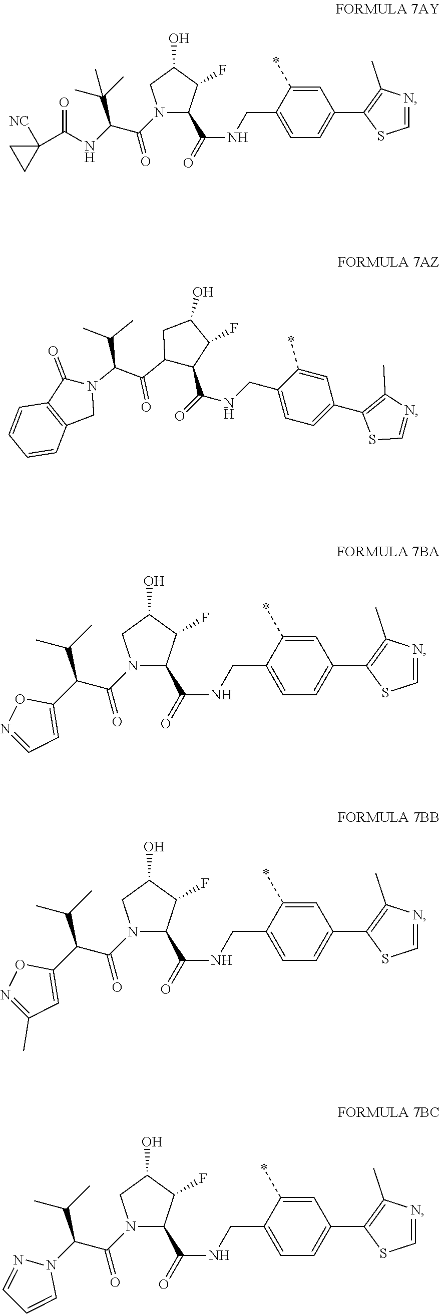

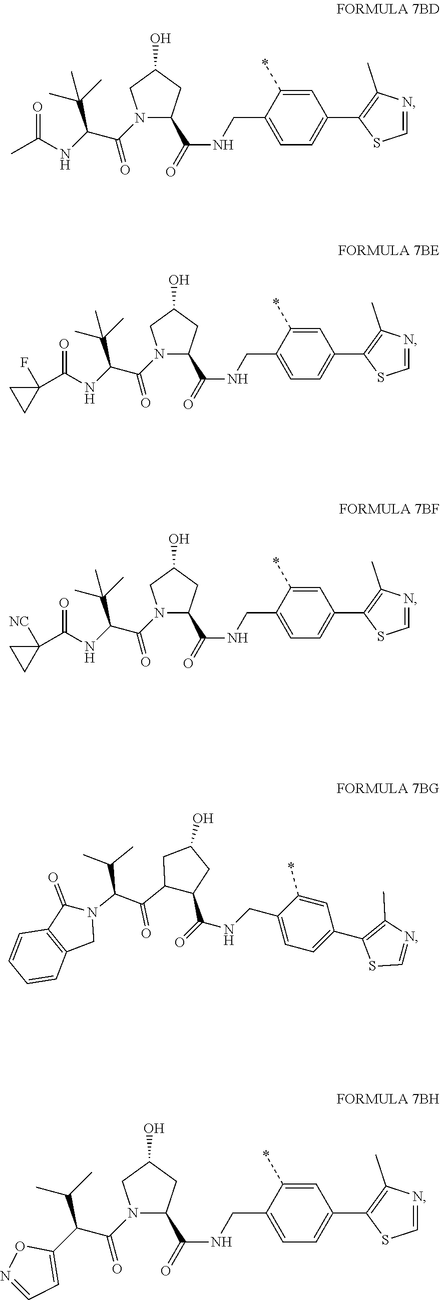

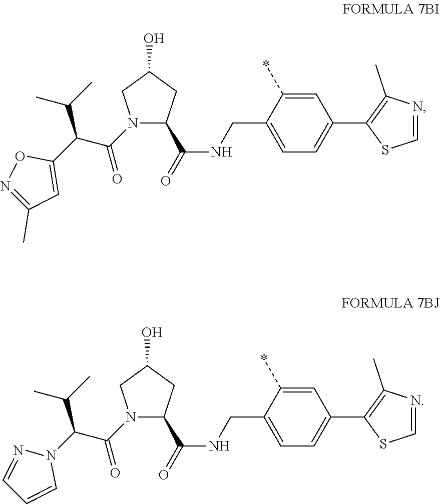

106. The heterobifunctional compound of claim 89, or a pharmaceutically acceptable salt thereof, wherein the Degradation tag is a moiety of FORMULA 5-1, or FORMULA 5-3, 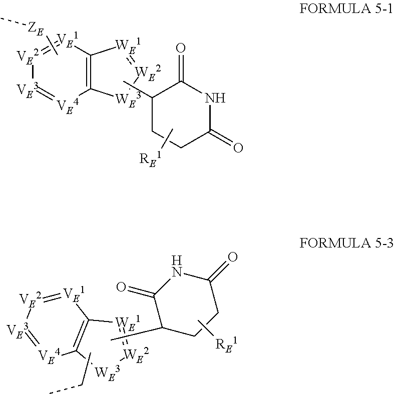
wherein
V_{E}¹, V_{E}², V_{E}³, and V_{E}⁴ are each independently selected from a bond, C, CR_{E}², and N; or V_{E}¹ and V_{E}², V_{E}² and V_{E}³, or V_{E}³ and V_{E}⁴ are combined together to optionally form 6 membered aryl ring or 5, 6 or 7 membered heteroaryl ring; [symbol]
indicates a single bond or a double bond; wherein (i) when there is a single bond between W_{E}¹ and W_{E}², W_{E}¹, W_{E}² and W_{E}³ are each independently selected from the group consisting of —N═, —CR_{E}³═, —CO—, —O—, —CR_{E}³R_{E}⁴—, —NR_{E}³—, —CR_{E}³═CR_{E}⁴—, —N═CR_{E}³—, and —N═N—; or (ii) when there is a double bond between W_{E}¹ and W_{E}², W_{E}¹ and W_{E}² are each independently selected from the group consisting of —N═, —C≡ and —CR_{E}³═; W_{E}³ is selected from the group consisting of —CR_{E}³R_{E}⁴—, —O—, —N═, —NR_{E}³—, —C(O)NR_{E}³—, —CR_{E}³═CR_{E}⁴—, and —CR_{E}³═N—;
Z_{E}, R_{E}², R_{E}³, R_{E}⁴ and R_{E}¹ are defined as in FORMULA 5.

107. The heterobifunctional compound of claim 106, or a pharmaceutically acceptable salt thereof, wherein the Degradation tag is a moiety of FORMULA 5A, 5B, 5E, 5F or 5G 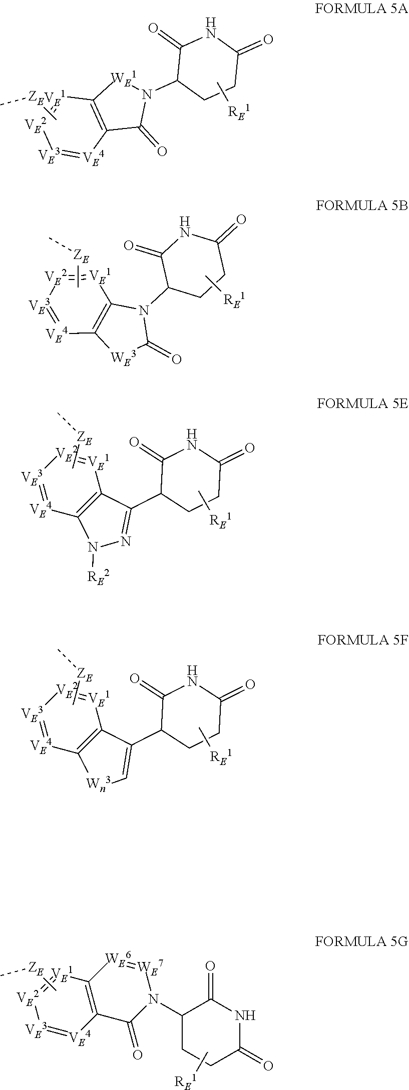
wherein W_{E}⁶ and W_{E}⁷ are each independently selected from —CR_{E}²═ and —N═; and V_{E}¹, V_{E}², V_{E}³, V_{E}⁴, W_{E}¹, W_{E}³, Z_{E}, R_{E}³ and R_{E}¹ are defined as in FORMULA 5-1.

108. The heterobifunctional compound of claim 107, or a pharmaceutically acceptable salt thereof, wherein the degradation tag is a moiety of FORMULA 5A.

109. The heterobifunctional compound of claim 89, or a pharmaceutically acceptable salt thereof, wherein the degradation tag is a moiety of FORMULAE 8A to 8AD:

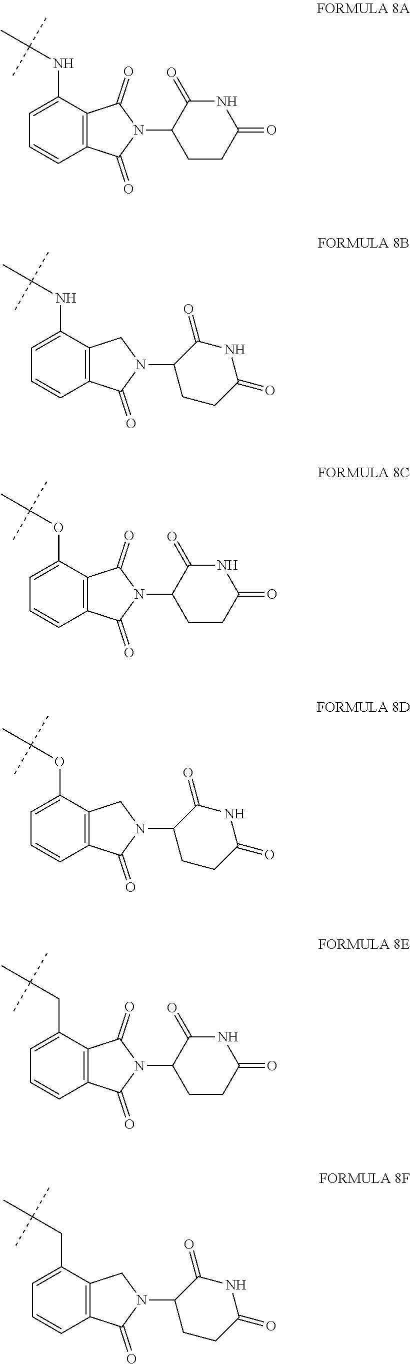

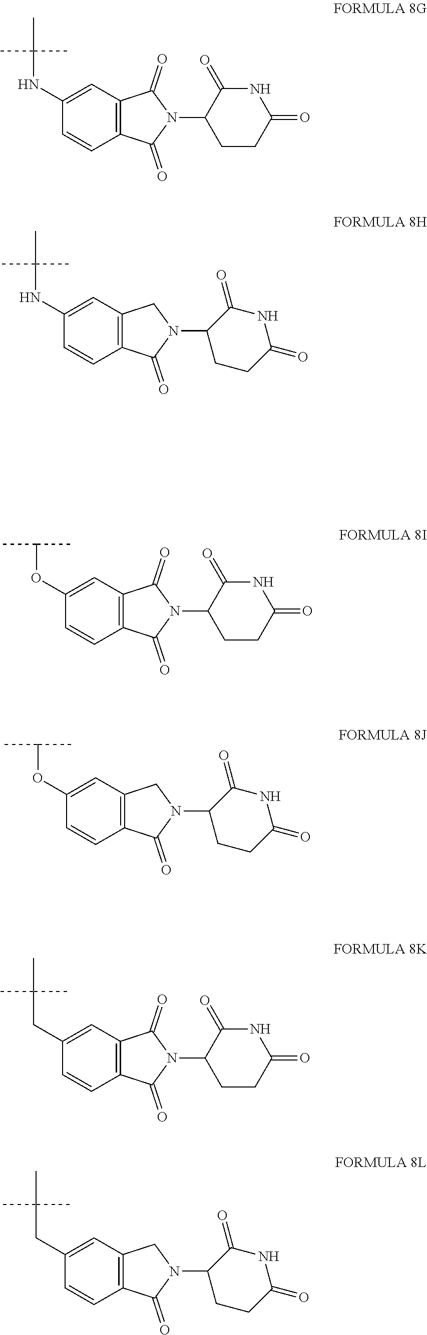

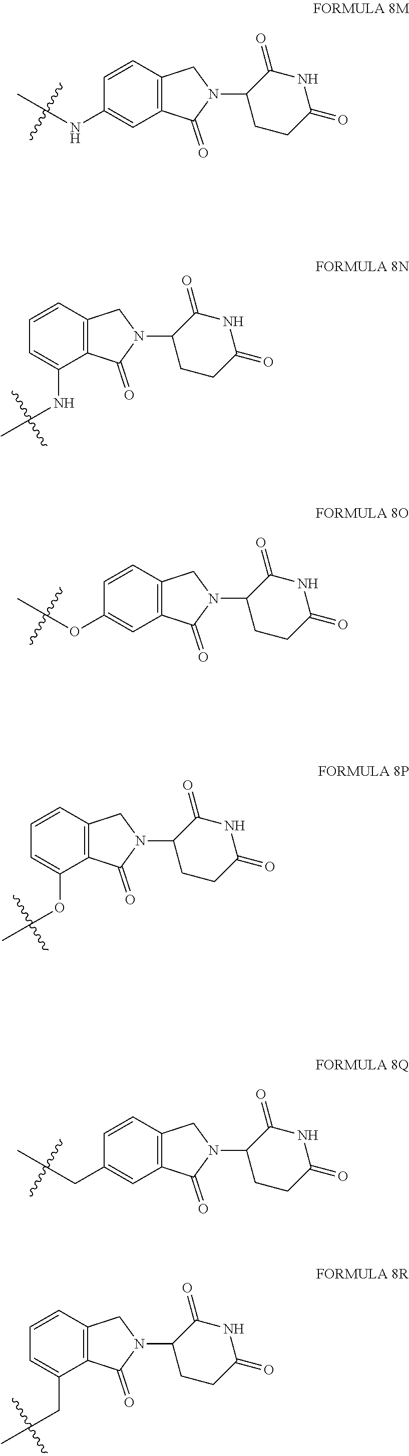 [SMILES: *Nc1ccc2c(c1)C(=O)N(C1CCC(=O)NC1=O)C2 ; *Nc1cccc2c1C(=O)N(C1CCC(=O)NC1=O)C2 ; *Oc1ccc2c(c1)C(=O)N(C1CCC(=O)NC1=O)C2 ; *Oc1cccc2c1C(=O)N(C1CCC(=O)NC1=O)C2 ; *Cc1ccc2c(c1)C(=O)N(C1CCC(=O)NC1=O)C2 ; *Cc1cccc2c1C(=O)N(C1CCC(=O)NC1=O)C2]

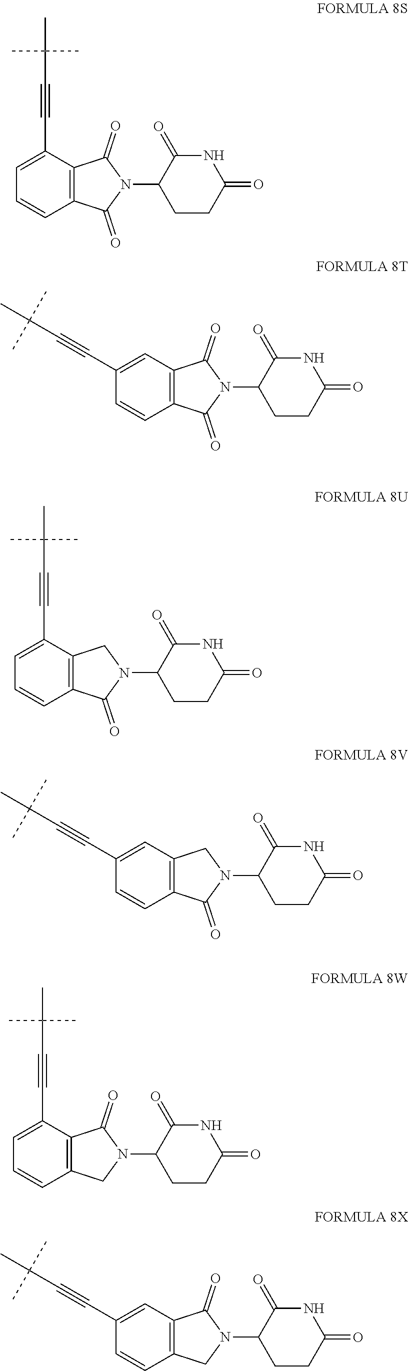

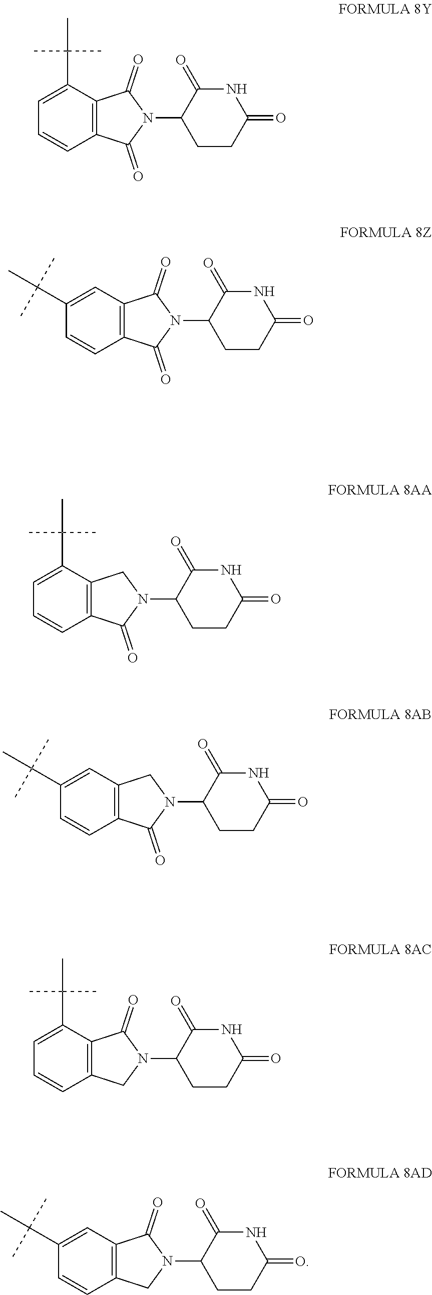

110. The heterobifunctional compound of claim 89, or a pharmaceutically acceptable salt thereof, wherein A_{L} and B_{L}, at each occurrence, are independently selected from the group consisting of null, R_{L}^{d}—R_{L}^{e}, R_{L}^{d}COR_{L}^{e}, R_{L}^{d}C(O)OR_{L}^{e}, R_{L}^{d}C(O)N(R_{L}¹)R_{L}^{e}, R_{L}^{d}OR_{L}^{e}, R_{L}^{d}SR_{L}^{e}, R_{L}^{d}N(R_{L}¹)R_{L}^{e}, R_{L}^{d}N(R_{L}¹)COR_{L}^{e}, wherein R_{L}^{d} and R_{L}^{e}, at each occurrence, are independently selected from the group consisting of null, optionally substituted C₁, C₂ or C₃ alkylene, R_{L}^{r}, R_{L}^{r}-(C₁, C₂ or C₃ alkylene), (C₁, C₂ or C₃ alkylene)-R_{L}^{r}, and (C₁, C₂ or C₁ alkylene)-R_{L}^{r}-(C₁, C₂ or C₃ alkylene).

111. The heterobifunctional compound of claim 89, or a pharmaceutically acceptable salt thereof, wherein W_{L}¹ and W_{L}², at each occurrence, are independently selected from null, O, S, NH, R_{L}^{r}, optionally substituted C₁-C₃ alkylene, with the proviso that at least one of W_{L}¹ and W_{L}² is not null.

112. The heterobifunctional compound of claim 89, or a pharmaceutically acceptable salt thereof, wherein A_{L} is the attachment to the TYK2 ligand;
A_{L} is selected from the group consisting of R_{L}—R_{L}^{e}, R_{L}^{d}C(O)R_{L}^{e}, R_{L}^{d}C(O)NHR_{L}^{e}, R_{L}^{d}NHC(O)R_{L}^{e}, R^{d}C(O)NHR_{L}^{e}, and R^{d}NHC(O)R_{L}^{e};
B_{L} is selected from the group consisting of null, R_{L}^{d}C(O)NHR_{L}^{e}, R_{L}^{d}C(O)R_{L}^{e}, R_{L}^{d}NHC(O)R_{L}^{e}, and R_{L}^{d}NHR_{L}^{e};
R_{L}^{d} and R_{L}^{e}, at each occurrence, are independently selected from the group consisting of null, optionally substituted C₁, C₂ or C₃ alkylene, R_{L}^{r}, R_{L}^{r}-(C₁, C₂ or C₃ alkylene), (C₁, C₂ or C₃ alkylene)-R_{L}^{r}, and (C₁, C₂ or C₃ alkylene)-R_{L}^{r}-(C₁, C₂ or C₁ alkylene);
W_{L}², at each occurrence, is independently selected from null, O, or NH; and W_{L}¹, at each occurrence, is independently selected from R_{L}^{r}, and optionally substituted C₁, C₂ or C₃ alkylene.

113. The heterobifunctional compound of claim 89, or a pharmaceutically acceptable salt thereof, wherein
1) R^{1′} is selected from the group consisting of C(O), C(O)—CH₂, 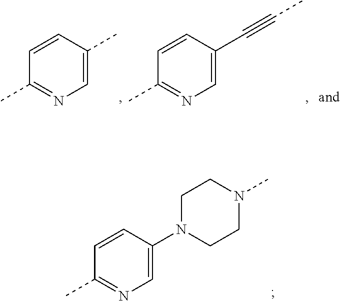
2) the degradation tag is a moiety of FORMULAE 6A; and
3) the linker moiety is of FORMULA 9, wherein A_{L} is the attachment to the TYK2 ligand; and wherein
A_{L} is selected from the group consisting of CO, NHCO, CONH, CH₂CONH, CH₂NHCO, 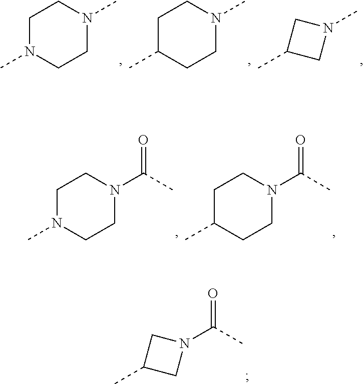
B_{L} is C(O);
W_{L}² is null, and W_{L}¹ is independently optionally substituted C₁ alkylene; and
m_{L} is 1, 2, 3, 4, 5, 6, 7, 8, 9, 10, 11, 12, 13, 14, or 15.

114. The heterobifunctional compound of claim 89, or a pharmaceutically acceptable salt thereof, wherein
1) R^{1′} is selected from the group consisting of C(O);
2) the degradation tag is a moiety of FORMULAE 6B or 6C; and
3) the linker moiety is of FORMULA 9, wherein A_{L} is the attachment to the TYK2 ligand; and wherein
A_{L} is selected from the group consisting of NHCO, CONH, CH₂CONH, CH₂NHCO;
B_{L} is C(O);
W_{L}² is null, and W_{L}¹ is independently optionally substituted C₁ alkylene; and
m_{L} is 1, 2, 3, 4, 5, 6, 7, 8, 9, 10, 11, 12, 13, 14, or 15.

115. The heterobifunctional compound of claim 106, or a pharmaceutically acceptable salt thereof, wherein
1) the degradation tag is a moiety of FORMULA 5-1; and
2) the linker moiety is of FORMULA 9, wherein
A_{L}, and B_{L}, at each occurrence, are bivalent moieties independently selected from the group consisting of null, R_{L}^{d}—R_{L}^{e}, R_{L}^{d}COR_{L}^{e}, R_{L}^{d}C(O)OR_{L}^{e}, R_{L}^{d}C(O)N(R_{L}¹)R_{L}^{e}, R_{L}^{d}C(S)N(R_{L}¹)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}¹)R_{L}^{e}, R_{L}^{d}N(R_{L}¹)R_{L}^{e}, R_{L}^{d}N(R_{L}¹)COR_{L}^{e}, R_{L}^{d}N(R_{L}¹)CON(R_{L}²)R_{L}^{e}, R_{L}^{d}N(R_{L}¹)C(S)R_{L}^{e}, optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ cycloalkyl, optionally substituted 3-13 membered heterocyclyl, optionally substituted aryl, and optionally substituted heteroaryl;
W_{L}¹ is independently optionally substituted C₁, C₂ or C₃ alkylene and W_{L}² is null or O; wherein R_{L}^{d}, R_{L}^{e}, R_{L}^{r}, R_{L}¹ and R_{L}² are defined above; and
m_{L} is 1, 2, 3, 4, 5, 6, 7, 8, 9, 10, 11, 12, 13, 14, or 15.

116. The heterobifunctional compound of claim 107, or a pharmaceutically acceptable salt thereof wherein
1) R^{1′} is optionally substituted pyridinyl;
2) the degradation tag is a moiety of FORMULA 5A; and
3) the linker moiety is of FORMULA 9, wherein A_{L} is the attachment to the TYK2 ligand; and wherein
A_{L} is selected from the group consisting of NHCO, and CONH;
B_{L} is null;
W_{L}¹ is independently optionally substituted C₁, C₂ or C₃ alkylene and W_{L}¹ is null or O;
R_{L}^{d}, R_{L}^{e}, R_{L}^{r}, R_{L}¹ and R_{L}² are defined in FORMULA 9; and
m_{L} is 1, 2, 3, 4, 5, 6, 7, 8, 9, 10, 11, 12, 13, 14, or 15.

117. The heterobifunctional compound of claim 107, or a pharmaceutically acceptable salt thereof, wherein
1) R^{1′} is selected from the group consisting of C(O) and C(O)—CH₂;
2) the degradation tag is a moiety of FORMULA 5A and Z_{E} is connected to V_{E}¹ or V_{E}⁴; and
3) the linker moiety is of FORMULA 9, wherein A_{L} is the attachment to the TYK2 ligand; and wherein
A_{L} is selected from the group consisting of CH₂NHCO, and CH₂CONH, NHCO, and CONH;
B_{L}, is null;
W_{L}¹ is independently optionally substituted C₁ alkylene and W_{L}² is null; and
m_{L} is 1, 2, 3, 4, 5, 6, 7, 8, 9, 10, 11, 12, 13, 14, or 15.

118. The heterobifunctional compound of claim 89, or a pharmaceutically acceptable salt thereof, wherein R_{L}^{r}, at each occurrence, is selected from FORMULAE C₁, C₂, C₃, C₄, and C₅: 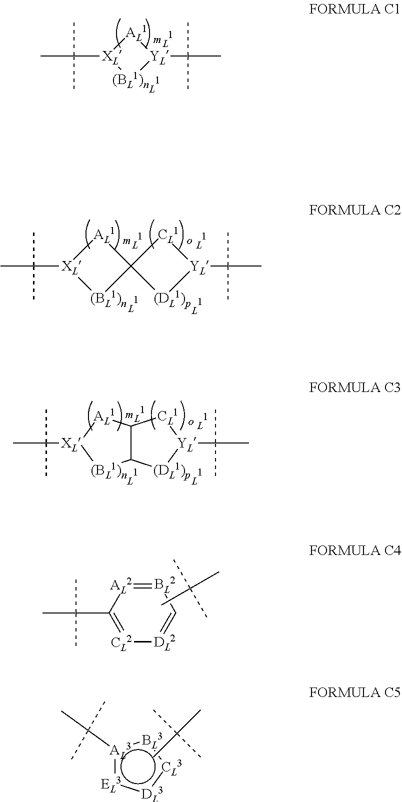
wherein
A_{L}¹, B_{L}¹, C_{L}¹ and D_{L}¹, at each occurrence, are independently selected from the group consisting of null, O, CO, SO, SO₂, NR_{L}^{b}, CR_{L}^{b}R_{L}^{c};
X_{L}′, Y_{L}′, A_{L}², B_{L}², C_{L}², D_{L}² and E_{L}², at each occurrence, are independently selected from N, CR_{L}^{b};
A_{L}³, B_{L}³, C_{L}³, D_{L}³, and E_{L}³, at each occurrence, are independently selected from N, O, S, NR_{L}^{b}, CR_{L}^{b};
R_{L}^{b} and R_{L}^{c}, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted C₃-C₁₀ carbocyclyl, optionally substituted C₃-C₁₀ cycloalkoxy, optionally substituted C₃-C₁₀ carbocyclylamino, optionally substituted 3-10 membered heterocyclyl, optionally substituted aryl, and optionally substituted heteroaryl; and
m_{L}¹, n_{L}¹, o_{L}¹ and p_{L}¹ are independently selected from 0, 1, 2, 3, 4 and 5.

119. The heterobifunctional compound of claim 89, or a pharmaceutically acceptable salt thereof, wherein R_{L}^{r}, at each occurrence, is selected from Group R_{L}^{r1} and Group R_{L}^{r2}, and
Group R_{L}^{r1} consists of optionally substituted following cyclic groups 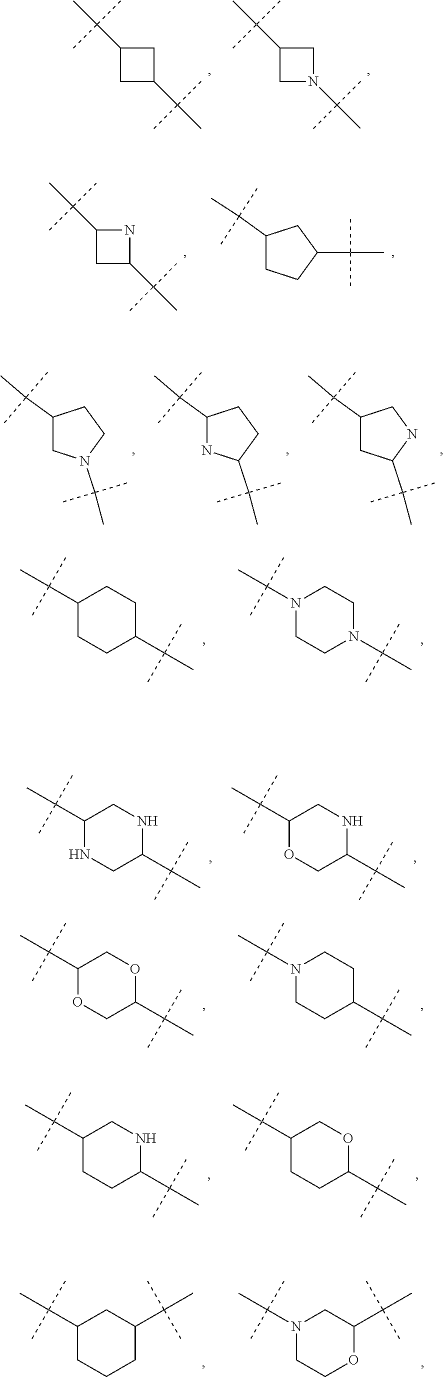 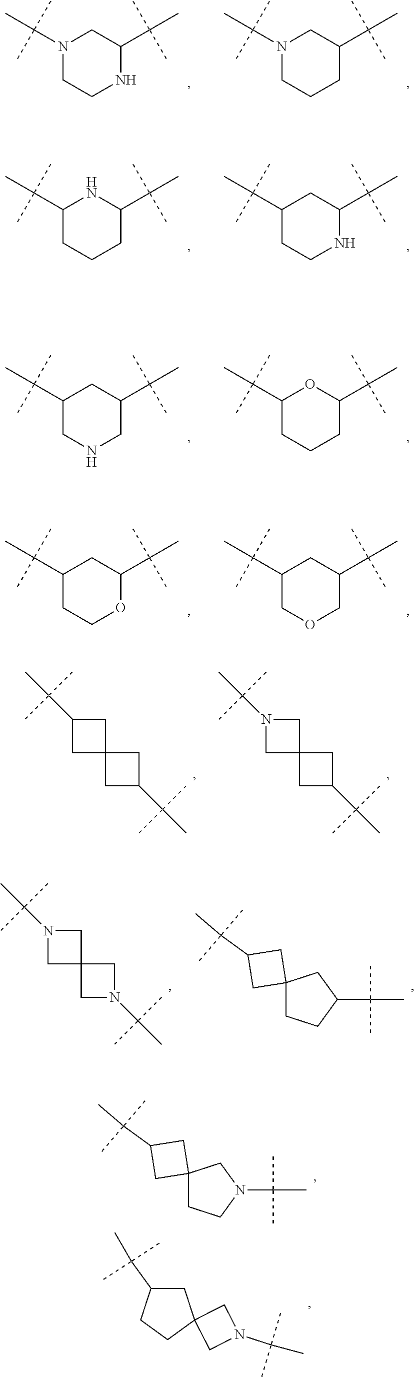 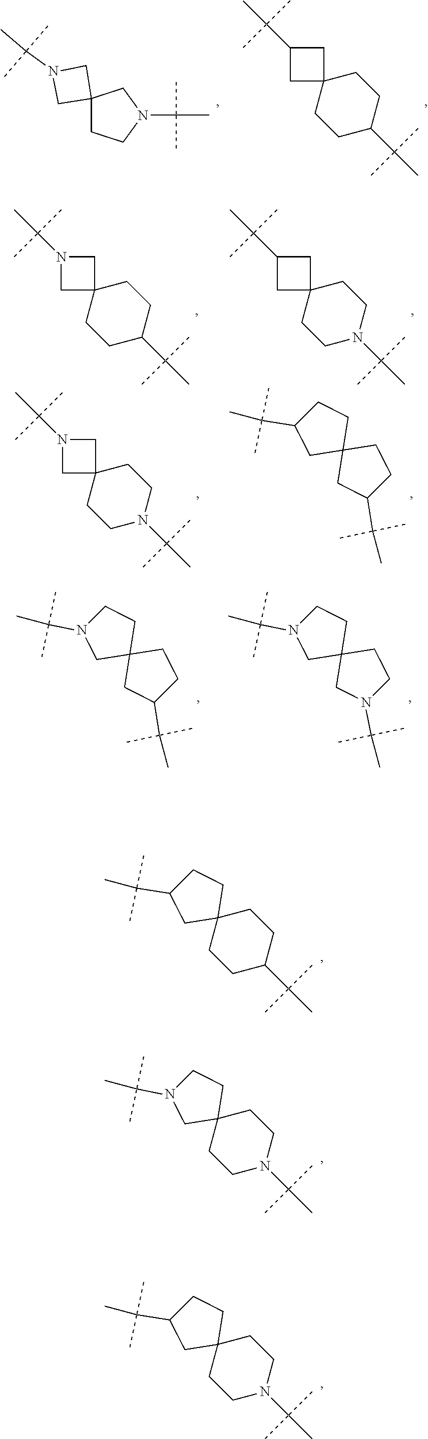 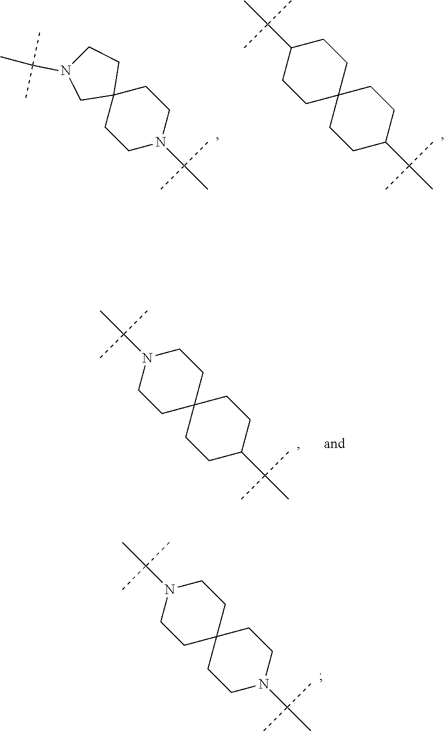
Group_{L}^{r2} consists of optionally substituted following cyclic groups

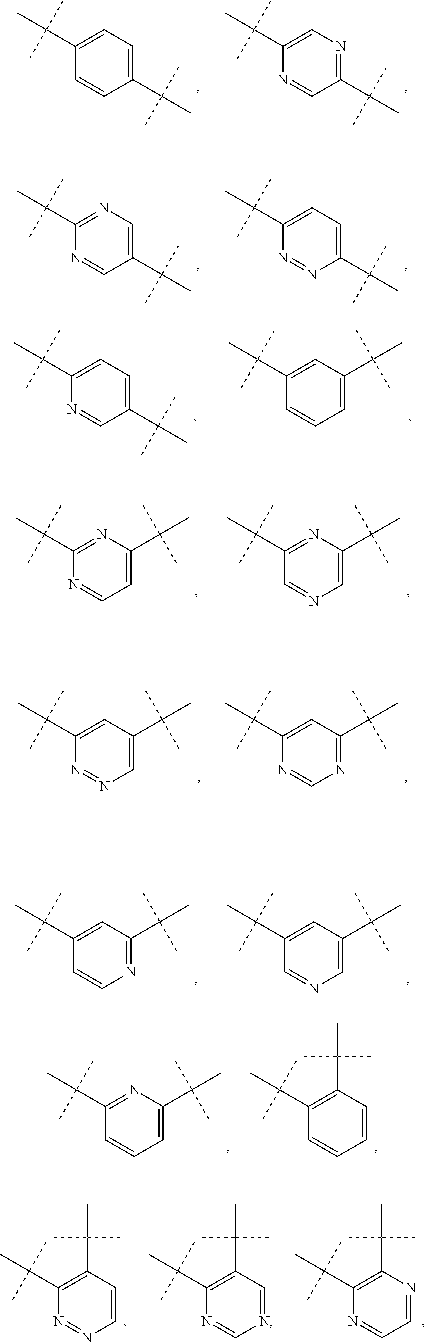

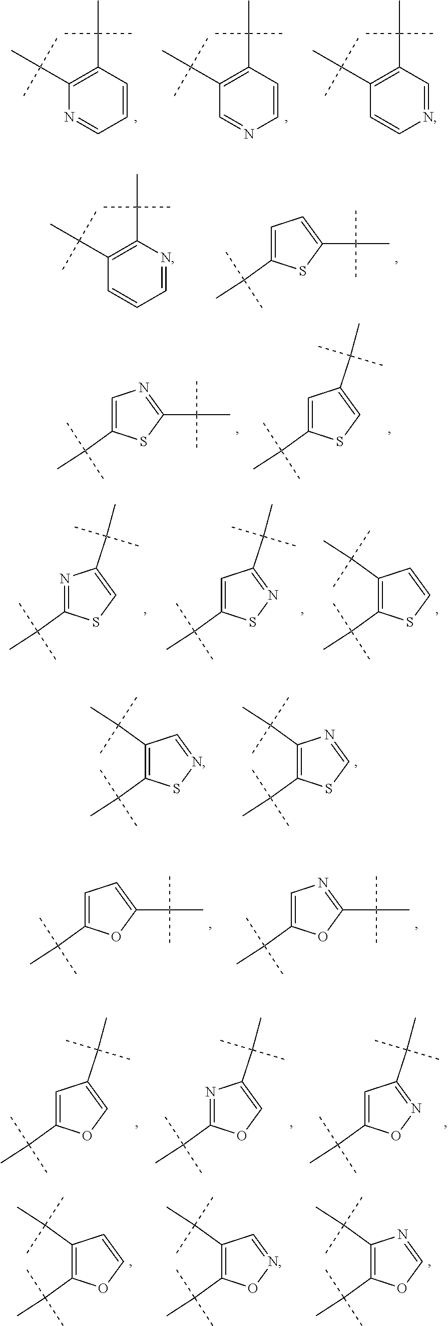

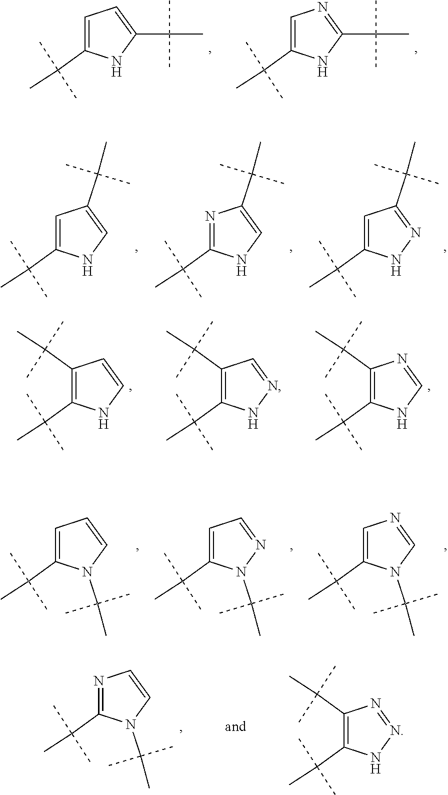

120. The compound of claim 89, wherein the compound is selected from the group consisting of
N1-((S)-1-((2S,4R)-4-hydroxy-((4-(4-methylthiazol-5-yl)benzyl)carbamoyl)pyrrolidin-1-yl)-3,3-dimethyl-1-oxobutan-2-yl)-N8-(2-((5-((2-methoxy-3-(1-methyl-1H-1,2,4-triazol-3-yl)phenyl)amino)-6-(methylcarbamoyl)pyridazin-3-yl)amino)-2-oxoethyl)octanediamide (CPD-038);
N1-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N9-(2-(5-((2-methoxy-3-(1-methyl-1H-1,2,4-triazol-3-yl)phenyl)amino)-6-(methylcarbamoyl)pyridazin-3-yl)amino)-2-oxoethyl)nonanediamide (CPD-039); and
N1-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N10-(2-((5-((2-methoxy-3-(1-methyl-1H-1,2,4-triazol-3-yl)phenyl)amino)-6-(methylcarbamoyl)pyridazin-3-yl)amino)-2-oxoethyl)decanediamide (CPD-040);
6-(2-(7-((2-(2,6-Dioxopiperidin-3-yl)-1,3-dioxoisoindolin-4-yl)amino)heptanamido)acetamido)-1-((2-methoxy-3-(1-methyl-1H-1,2,4-triazol-3-yl)phenyl)amino)-N-methylpyridazine-3-carboxamide (CPD-047);
N1-((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)-N9-(2-((5-((2-methoxy-3-(1-methyl-1H-1,2,4-triazol-3-yl)phenyl)amino)-6-(methylcarbamoyl)pyridazin-3-yl)amino)-2-oxoethyl)nonanediamide (CPD-084);
N1-((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)-N10-(2-((5-((2-methoxy-3-(1-methyl-1H-1,2,4-triazol-3-yl)phenyl)amino)-6-(methylcarbamoyl)pyridazin-3-yl)amino)-2-oxoethyl)decanediamide (CPD-085);
6-((5-(4-(7-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-7-oxoheptanoyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-099);
6-((5-(4-(8-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-8-oxooctanoyl)piperazin-1-yl)pyridin-2-yl)amino)-4-((2-methoxy-3-(1-methyl-1H-1,2,4-triazol-3-yl)phenyl)amino)-NV-methylpyridazine-3-carboxamide (CPD-100);
6-(2-(11-(2-(((2S,4R)-1-((S)-2-(1-Fluorocyclopropane-1-carboxamido)-3,3-dimethylbutanoyl)-4-hydroxypyrrolidine-2-carboxamido)methyl)-5-(4-methylthiazol-5-yl)phenoxy)undecanamido)acetamido)-4-((2-methoxy-3-(1-methyl-1H-1,2,4-triazol-3-yl)phenyl)amino)-NV-methylpyridazine-3-carboxamide (CPD-110);
6-((5-(4-(8-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctanoyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-112);
6-((5-(4-(10-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-114);
6-((5-(4-(9-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononanoyl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-115);
6-((5-((1-(10-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoyl)piperidin-4-yl)ethynyl)pyridin-2-yl)amino)-4-((2-methoxy-3-(1-methyl-1H-1,2,4-triazol-3-yl)phenyl)amino)-N-methylpyridazine-3-carboxamide (CPD-121);
6-((5-((1-(7-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoyl)azetidin-3-yl)ethynyl)pyridin-2-yl)amino)-4-((2-methoxy-3-(1-methyl-1H-1,2,4-triazol-3-yl)phenyl)amino)-N-methylpyridazine-3-carboxamide (CPD-124);
6-((5-((1-(8-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctanoyl)azetidin-3-yl)ethynyl)pyridin-2-yl)amino)-4-((2-methoxy-3-(1-methyl-1H-1,2,4-triazol-3-yl)phenyl)amino)-N-methylpyridazine-3-carboxamide (CPD-125);
6-((5-((1-(9-((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononanoyl)azetidin-3-yl)ethynyl)pyridin-2-yl)amino)-4-((2-methoxy-3-(1-methyl-1H-1,2,4-triazol-3-yl)phenyl)amino)-N-methylpyridazine-3-carboxamide (CPD-126);
6-((5-((1-(10-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoyl)azetidin-3-yl)ethynyl)pyridin-2-yl)amino)-4-((2-methoxy-3-(1-methyl-1H-1,2,4-triazol-3-yl)phenyl)amino)-N-methylpyridazine-3-carboxamide (CPD-127);
1-((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)-N8-((6-((5-((2-methoxy-3-(1-methyl-1H-1,2,4-triazol-3-yl)phenyl)amino)-6-(methylcarbamoyl)pyridazin-3-yl)amino)pyridin-3-yl)methyl)octanediamide (CPD-131);
N1-((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)-N10-((6-((5-((2-methoxy-3-(1-methyl-ill-1,2,4-triazol-3-yl)phenyl)amino)-6-(methylcarbamoyl)pyridazin-3-yl)amino)pyridin-3-yl)methyl)decanediamide (CPD-133);
6-((5-((1-(5-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentanoyl)piperidin-4-yl)ethynyl)pyridin-2-yl)amino)-4-((2-methoxy-3-(1-methyl-1H-1,2,4-triazol-3-yl)phenyl)amino)-N-methylpyridazine-3-carboxamide (CPD-134);
6-((5-((8-(((S-1-((2S,4R)-4-Hydroxy-2-((S)-1-(4-(4-methylthiazol-5-yl)phenyl)ethyl)carbamoyl)pyrrolidin-1-yl)-3,3-dimethyl-1-oxobutan-2-yl)amino)-8-oxooctyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-143);
6-((5-((9-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-144);
6-((5-(4-(1-(7-(((S)-1-((2S,41R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoyl)piperidin-4-yl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-148);
6-((5-(4-(1-(9-(((S)-1-((2S,4R)-4-Hydroxy-2-(((S)-(4-(4-methylthiazol-5-yl)phenyl)ethyl)carbamoyl)pyrrolidin-1-yl)-3,3-dimethyl-1-oxobutan-2-yl)amino)-9-oxononanoyl)piperidin-4-yl)piperazin-1-yl)pyridin-2-yl)amino)-4-((2-methoxy-3-(1-methyl-1H-1,2,4-triazol-3-yl)phenyl)amino)-N-methylpyridazine-3-carboxamide (CPD-150);
6-((5-(4-(1-(10-(((S)-1-(2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoyl)piperidin-4-yl)piperazin-1-yl)pyridin-2-yl)amino)-4-((2-methoxy-3(1i-methyl-ill-1,2,4-triazol-3-yl)phenyl)amino)-N-methylpyridazine-3-carboxamide (CPD-151);
6-((5-((5-((2-(2,6-Dioxopiperidin-3-yl)-1,3-dioxoisoindolin-4-yl)amino)pentyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-155);
6-((5-(7-((2-(2,6-Dioxopiperidin-3-yl)-1,3-dioxoisoindolin-4-yl)amino)heptyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-157);
6-((5-((8-((2-(2,6-Dioxopiperidin-3-yl)-1,3-dioxoisoindolin-4-yl)amino)octyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-158);
6-((5-((2-(2-((2-(2,6-Dioxopiperidin-3-yl)-1,3-dioxoisoindolin-4-yl)amino)ethoxy)ethyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-159);
6-((5-((5-((2-(2,6-Dioxopiperidin-3-yl)-1, 3-dioxoisoindolin-5-yl)amino)pentyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-164);
6-((5-((8-((2-(2,6-Dioxopiperidin-3-yl)-1,3-dioxoisoindolin-5-yl)amino)octyl)carbamoyl)pyridin-2-yl)amino)-4-((2-methoxy-3-(1-methyl-1H-1,2,4-triazol-3-yl)phenyl)amino)-N-methylpyridazine-3-carboxamide (CPD-167); and
6-((5-((3-((2-(2,6-Dioxopiperidin-3-yl)-1,3-dioxoisoindolin-5-yl)amino)propyl)carbamoyl)pyridin-2-yl)amino)-4-((2-methoxy-3-(1-methyl-1H-1,2,4-triazol-3-yl)phenyl)amino)-NV-methylpyridazine-3-carboxamide (CPD-175);
or a pharmaceutically acceptable salt thereof.

121. A pharmaceutical composition comprising a compound of claim 89, or a pharmaceutically acceptable salt thereof, and one or more pharmaceutically acceptable carriers.

122. A method of treating a TYK2-mediated disease, wherein the method comprises administering to a subject in need thereof an effective amount of a heterobifunctional compound of claim 89, or a pharmaceutically acceptable salt thereof.

123. The method of claim 122, wherein the method further comprises administering to the subject an additional therapeutic regimen for treating cancer, inflammatory disorders, and/or autoimmune diseases.

124. The method of claim 123, wherein the additional therapeutic regimen is selected from the group consisting of surgery, chemotherapy, radiation therapy, hormone therapy, targeted therapy, and immunotherapy.

125. The method of claim 122, wherein the TYK2-mediated disease is selected from the group consisting of cancer, inflammatory disorders, auto-immune diseases, dermatological disorders, viral infections, dry eye disorders, bone remodeling disorders, and organ transplant associated immunological complications, or a combination thereof.
